(19) United States
(12) Patent Application Publication
(10) Pub. No.: US 2024/0270741 A1
(43) Pub. Date: Aug. 15, 2024

(54) COMPOUNDS FOR FGFRS INHIBITORS

(71) Applicant: Cogent Biosciences, Inc., Waltham, MA (US)

(72) Inventors: Jay Bradford Fell, Boulder, CO (US); John E. Robinson, Boulder, CO (US); John P. Fischer, Boulder, CO (US); Logan E. Vine, Boulder, CO (US); Martha E. Rodriguez, Boulder, CO (US); Jennifer Fulton, Boulder, CO (US); Tanna Bettendorf, Boulder, CO (US); Bradley J. Newhouse, Boulder, CO (US); Robert A. Rieger, Boulder, CO (US); Cori A. Malinky, Boulder, CO (US); Aaron Christopher Smith, Boulder, CO (US); Toya D. Scaggs, Boulder, CO (US); Joshua Dahlke, Boulder, CO (US); Ravi Kumar Jalluri, Boulder, CO (US); Mark Joseph Chicarelli, Boulder, CO (US); Leah J. Salituro, Boulder, CO (US); Macedonio J. Mejia, Boulder, CO (US); Payal Chatterjee, Boulder, CO (US)

(21) Appl. No.: 18/408,088

(22) Filed: Jan. 9, 2024

Related U.S. Application Data
(60) Provisional application No. 63/479,292, filed on Jan. 10, 2023.
(60) Provisional application No. 63/516,701, filed on Jul. 31, 2023.

(51) Int. Cl. C07D 471/04; A61K 31/437; A61K 31/438; A61K 31/439; A61K 31/4545; A61K 31/496; A61K 31/4985; A61K 31/506; A61K 31/5377; A61K 31/541; A61K 31/661; C07B 59/00; C07D 519/00; C07F 9/6561

(52) U.S. Cl. CPC C07D 471/04; A61K 31/437; A61K 31/438; A61K 31/439; A61K 31/4545; A61K 31/496; A61K 31/4985; A61K 31/506; A61K 31/5377; A61K 31/541; A61K 31/661; C07B 59/002; C07D 519/00; C07F 9/6561; C07B 2200/05

(57) ABSTRACT

A compound having the following structure of Formula (I):

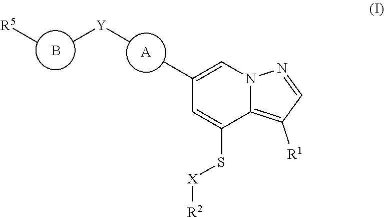

or a stereoisomer, salt, or tautomer thereof, wherein R¹, R², R⁵, A, B, X, and Y are as defined herein. Pharmaceutical composition comprising the compounds, and their use in methods of treating diseases are also described.

### CROSS-REFERENCE TO RELATED APPLICATIONS

This application claims priority to U.S. Provisional Patent Application Nos. 63/479,292, filed on Jan. 10, 2023, and 63/516,701, filed Jul. 31, 2023, the contents of each of which are incorporated by reference herein in their entirety.

### TECHNICAL FIELD

The disclosure relates to substituted pyrazolo pyridine compounds that act as fibroblast growth factor receptor tyrosine kinases (FGFRs) inhibitors. The disclosure also provides compounds of formula (I) and pharmaceutically acceptable salts thereof and uses of the compounds for the treatment of abnormal cell growth, such as cancer, in a subject.

### BACKGROUND

Fibroblast growth factors (FGFs) and their receptors (FGFRs) regulate a wide range of physiologic cellular processes, such as embryonic development, differentiation, proliferation, survival, migration, and angiogenesis. There are five FGFRs, of which four (FGFRs 1-4) are highly conserved single-pass transmembrane tyrosine kinase receptors. The binding of an FGF to an FGFR leads to receptor dimerization and transphosphorylation of tyrosine kinase domains. Dysregulation of the FGF signaling system underlies a range of diseases associated with the increased FGF expression.

There remains a need to discover FGFRs inhibitors having enhanced activity profiles which may be useful for the treatment of FGFRs mutation cancers or other proliferative diseases or conditions.

### BRIEF SUMMARY

In brief, the present disclosure provides compounds, including stereoisomers, pharmaceutically acceptable salts, or tautomers thereof, which can be used alone or in combination with other therapeutic agents.

In one embodiment, a compound having a structure of Formula (I) is provided:

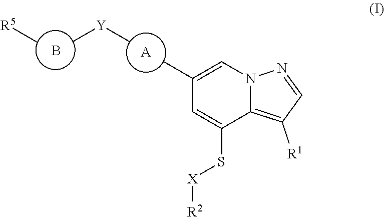

or a stereoisomer, salt, or tautomer thereof, R¹, R², R⁵, A, B, X, and Y are as defined herein.

Pharmaceutical compositions comprising one or more of the foregoing compounds of Formula (I) and a therapeutic agent are also provided.

In other embodiments, methods of treatment by administering the foregoing compounds of Formula (I) or the pharmaceutical compositions comprising a compound of Formula (I), to a subject in need thereof to treat a disease is provide.

Various aspects and embodiments now will be described more fully hereinafter. Such aspects and embodiments make take many different forms and the exemplary ones disclosed herein should not be construed as limiting; rather, these embodiments are provided so that this disclosure will be thorough and complete, and will fully convey its scope to those skilled in the art.

### DETAILED DESCRIPTION

### I. Definitions

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

The singular forms “a,” “an,” and “the” include plural referents unless the context clearly dictates otherwise. Thus, for example, reference to a “polymer” includes a single polymer as well as two or more of the same or different polymers, reference to an “excipient” includes a single excipient as well as two or more of the same or different excipients, and the like.

The term “about”, particularly in reference to a given quantity, is meant to encompass deviations of plus or minus five percent.

The compositions of the present disclosure can comprise, consist essentially of, or consist of, the components disclosed.

All percentages, parts and ratios are based upon the total weight of the compositions and all measurements made are at about 25° C., unless otherwise specified.

As used in the specification and appended claims, unless specified to the contrary, the following terms have the meaning indicated:

“Amino” refers to the —NH₂, —NHR, or —NR₂ radical.

“Cyano” refers to the —CN radical.

“Hydroxyl” refers to the —OH radical.

“Imino” refers to the ═NH or ═NR substituent.

“Nitro” refers to the —NO₂ radical.

“Oxo” refers to the ═O substituent.

“Thio” refers to the ═S substituent.

“Trifluoromethyl” refers to the —CF₃ radical.

Hyrazido or hydrazino refers to N—N substituent.
wherein each R is a compatible substituent as described in this disclosure. Where an R group is chiral, isomers are contemplated and included herein.

“Alkyl” refers to a linear, saturated, acyclic, monovalent hydrocarbon radical or branched, saturated, acyclic, monovalent hydrocarbon radical, having from one to twelve carbon atoms, preferably one to eight carbon atoms or one to six carbon atoms, and which is attached to the rest of the molecule by a single bond, e.g., methyl, ethyl, n-propyl, 1-methylethyl (iso-propyl), n-butyl, n-pentyl, 1,1-dimethylethyl (t-butyl), 3-methylhexyl, 2-methylhexyl and the like. An optionally substituted alkyl radical is an alkyl radical that is optionally substituted, valence permitting, by one, two, three, four, or five substituents independently selected from the group consisting of halo, cyano, nitro, aryl, cycloalkyl, heterocyclyl, heteroaryl, oxo, trimethylsilanyl, —OR′, —OC(O)—R′, —N(R′)₂, —C(O)R″, —C(O)OR′, —C(O)N(R′)₂, —N(R′)C(O)OR′″, —N(R′)C(O)R′″, —N(R′)S(O)ₜR′″ (where t is 1 or 2), —S(O)ₜOR′″ (where t is 1 or 2), —S(O)ₚR′″ (where p is 0, 1, or 2) and —S(O)^{t}N(R′)₂ (where t is 1 or 2), where each R′ is independently hydrogen, alkyl, haloalkyl, cycloalkyl, cycloalkylalkyl, aryl, aralkyl, heterocyclyl, or heteroaryl; each R″ is independently hydrogen, cycloalkyl, aryl, heterocyclyl, or heteroaryl; and each R′″ is independently alkyl, haloalkyl, cycloalkyl, aryl, heterocyclyl, or heteroaryl.

“Alkoxy” refers to a radical of the formula —ORₐ where Rₐ is an alkyl radical as defined above containing one to twelve carbon atoms. The alkyl part of the optionally substituted alkoxy radical is optionally substituted as defined above for an alkyl radical.

“Alkoxyalkyl” refers to a radical of the formula —Rₐ—O—R_{b} where Rₐ is alkylene and R_{b} is alkyl as defined above. Alkyl and alkylene parts of the optionally substituted alkoxyalkyl radical are optionally substituted as defined above for an alkyl radical and alkylene chain, respectively.

“Aralkyl” refers to a radical of the formula —Rₐ—R_{b}, where Rₐ is alkylene and R_{b} is aryl as described herein. Alkylene and aryl portions of optionally substituted aralkyl are optionally substituted as described herein for alkylene and aryl, respectively.

“Aryl” refers to an aromatic monocyclic or multicyclic hydrocarbon ring system radical containing from 6 to 18 carbon atoms, where the multicyclic aryl ring system is a bicyclic, tricyclic, or tetracyclic ring system. Aryl radicals include, but are not limited to, groups such as fluorenyl, phenyl and naphthyl. An optionally substituted aryl is an aryl radical that is optionally substituted by one, two, three, four, or five substituents independently selected from the group consisting of alkyl, akenyl, halo, haloalkyl, haloalkenyl, cyano, nitro, aryl, heteroaryl, heteroarylalkyl, —R″—OR′, —R″—OC(O)—R′, —R″—N(R′)₂, —R″—C(O)R′, —R″—C(O)OR′, —R″—C(O)N(R′)₂, —R″—N(R′)C(O)OR′″, —R″—N(R′)C(O)R′″, —R″—N(R′)S(O)ₜR′″ (where t is 1 or 2), —R″—S(O)ₜOR′″ (where t is 1 or 2), —R″—S(O)ₚR′″ (where p is 0, 1, or 2), and —R″—S(O)ₜN(R′)₂ (where t is 1 or 2), where each R′ is independently hydrogen, alkyl, haloalkyl, cycloalkyl, aryl, heterocyclyl, or heteroaryl; each R″ is independently a direct bond or a linear or branched alkylene or alkenylene chain; and each R′″ is independently alkyl, haloalkyl, cycloalkyl, cycloalkylalkyl, aryl, heterocyclyl, or heteroaryl.

“Arylalkoxy” refers to a group of formula —O—R, where R is aralkyl. An optionally substituted arylalkoxy is an arylalkoxy that is optionally substituted as described herein for aralkyl. In some embodiments, arylalkoxy is benzyloxy.

“Cycloalkyl” refers to a stable non-aromatic monocyclic or polycyclic hydrocarbon radical having from three to fifteen carbon atoms, preferably having from three to ten carbon atoms, and which is saturated or unsaturated, and which attaches to the rest of the molecule by a single bond. A polycyclic hydrocarbon radical is bicyclic, tricyclic, or tetracyclic ring system. An unsaturated cycloalkyl contains one, two, or three carbon-carbon double bonds and/or one carbon-carbon triple bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, and the like. An optionally substituted cycloalkyl is a cycloalkyl radical that is optionally substituted by one, two, three, four, or five substituents independently selected from the group consisting of alkyl, alkenyl, halo, haloalkyl, haloalkenyl, cyano, nitro, oxo, aryl, aralkyl, cycloalkyl, heterocyclyl, heteroaryl, —R″—OR′, —R″—OC(O)—R′, —R″—N(R′)₂, —R″—C(O)R′, —R″—C(O)OR′, —R″—C(O)N(R′)₂, —R″—N(R′)C(O)OR″, —R″—N(R′)C(O)R′″, —R″—N(R′)S(O)ₜR′″ (where t is 1 or 2), —R″—S(O)ₜOR′″ (where t is 1 or 2), —R″—S(O)ₚR′″ (where p is 0, 1, or 2) and —R″—S(O)ₜN(R′)₂ (where t is 1 or 2) where each R′ is independently hydrogen, alkyl, haloalkyl, cycloalkyl, aryl, heterocyclyl, or heteroaryl; each R″ is independently a direct bond or a linear or branched alkylene or alkenylene chain; and each R′″ is independently alkyl, haloalkyl, cycloalkyl, cycloalkylalkyl, aryl, aralkyl, heterocyclyl, or heteroaryl.

“Deuterated compounds” are compounds where one of more hydrogen atoms have been replaced with a deuterium atom. Deuterated drugs may be derivatives of an active compound. Deuterated drugs may be prodrugs. Deuteration may alter the physical properties, metabolic properties, activity or safety of a drug.

“Derivatives” are related chemical species that can be derived from a similar compound via chemical reactions. They may encompass slight chemical modifications, substitution of atoms with deuterated atoms, substitution of atoms with stable or radioactive isotopes or other modifications that imbue a compound with desirable properties.

“Fused” refers to any ring system described herein which is fused to an existing ring structure in the compounds of the invention. When the fused ring system is a heterocyclyl or a heteroaryl, any carbon atom on the existing ring structure which becomes part of the fused ring system may be replaced with a nitrogen atom.

“Halo” refers to the halogen substituents: bromo, chloro, fluoro, and iodo.

“Haloalkyl” refers to an alkyl radical, as defined above, that is further substituted by one or more halogen substituents. The number of halo substituents included in haloalkyl is from one and up to the total number of the hydrogen atoms available for replacement with the halo substituents (e.g., perfluoroalkyl). Non-limiting examples of haloalkyl include trifluoromethyl, difluoromethyl, trichloromethyl, 2,2,2-trifluoroethyl, 1-fluoromethyl-2-fluoroethyl, 3-bromo-2-fluoropropyl, 1-bromomethyl-2-bromoethyl and the like. For an optionally substituted haloalkyl, the hydrogen atoms bonded to the carbon atoms of the alkyl part of the haloalkyl radical may be optionally replaced with substituents as defined above for an optionally substituted alkyl.

“Haloalkenyl” refers to an alkenyl radical, as defined above, that is further substituted by one or more halo substituents. The number of halo substituents included in haloalkenyl is from one and up to the total number of the hydrogen atoms available for replacement with the halo substituents (e.g., perfluoroalkenyl). Non-limiting examples of haloalkenyl include 2,2-difluoroethenyl, 3-chloroprop-1-enyl, and the like. For an optionally substituted haloalkenyl, the hydrogen atoms bonded to the carbon atoms of the alkenyl part of the haloalkenyl radical may be optionally replaced with substitutents as defined above for an optionally substituted alkenyl group.

“Haloalkynyl” refers to an alkynyl radical, as defined above, that is further substituted by one or more halo substituents. The number of halo substituents included in haloalkynyl is from one and up to the total number of the hydrogen atoms available for replacement with the halo substituents (e.g., perfluoroalkynyl). Non-limiting examples of haloalkynyl include 3-chloroprop-1-ynyl and the like. The alkynyl part of the haloalkynyl radical may be additionally optionally substituted as defined above for an alkynyl group.

“Heteroarylalkyl” refers to a radical of the formula —Rₐ—R_{b}, where Rₐ is alkylene and R_{b} is heteroaryl as described herein. Alkylene and heteroaryl portions of optionally substituted heteroarylalkyl are optionally substituted as described herein for alkylene and heteroaryl, respectively.

“Heterocyclyl” refers to a stable 3- to 18-membered non-aromatic ring system radical having the carbon count of two to twelve and containing a total of one to six heteroatoms independently selected from the group consisting of nitrogen, oxygen, phosphorus, and sulfur. A heterocyclyl radical is a monocyclic, bicyclic, tricyclic, or tetracyclic ring system. A bicyclic, tricyclic, or tetracyclic heterocyclyl is a fused, spiro, and/or bridged ring system. The heterocyclyl radical may be saturated or unsaturated. An unsaturated heterocyclyl contains one, two, or three carbon-carbon double bonds and/or one carbon-carbon triple bond. An optionally substituted heterocyclyl is a heterocyclyl radical that is optionally substituted by one, two, three, four, or five substituents independently selected from the group consisting of alkyl, alkenyl, halo, haloalkyl, haloalkenyl, cyano, oxo, thioxo, nitro, aryl, aralkyl, cycloalkyl, heterocyclyl, heteroaryl, —R″—OR′, —R″—OC(O)—R′, —R″—N(R′)₂, —R″—C(O)R′, —R″—C(O)OR′, —R″—C(O)N(R′)₂, —R″—N(R′)C(O)OR′″, —R″—N(R′)C(O)R′″, —R″—N(R′)S(O)ₜR′″ (where t is 1 or 2), —R″—S(O)ₜOR′″ (where t is 1 or 2), —R″—S(O)ₚR′″ (where p is 0, 1, or 2), and —R″—S(O)ₜN(R′)₂ (where t is 1 or 2), where each R′ is independently hydrogen, alkyl, alkenyl, haloalkyl, cycloalkyl, aryl, heterocyclyl, or heteroaryl; each R″ is independently a direct bond or a linear or branched alkylene or alkenylene chain; and each R′″ is independently alkyl, alkenyl, haloalkyl, cycloalkyl, aryl, heterocyclyl, or heteroaryl. The nitrogen, carbon, or sulfur atoms in the heterocyclyl radical may be optionally oxidized (when the substituent is oxo and is present on the heteroatom); the nitrogen atom may be optionally quaternized (when the substituent is alkyl, alkenyl, aryl, aralkyl, cycloalkyl, heterocyclyl, heteroaryl, —R″—OR′, —R″—OC(O)—R′, —R″—N(R′)₂, —R″—C(O)R′, —R″—C(O)OR′, —R″—C(O)N(R′)₂, —R″—N(R′)C(O)OR′″, —R″—N(R′)C(O)R′″, —R″—N(R′)S(O)ₜR′″ (where t is 1 or 2), —R″—S(O)ₜOR′″ (where t is 1 or 2), —R″—S(O)ₚR′″ (where p is 0, 1, or 2), and —R″—S(O)ₜN(R′)₂ (where t is 1 or 2), where R″ is a linear or branched alkylene or alkenylene chain, and R′ and R′″ are as defined above). Examples of optionally substituted heterocyclyl radicals include, but are not limited to, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl.

“Heterocyclylene” refers to a heterocyclyl in which one hydrogen atom is replaced with a valency. An optionally substituted heterocyclylene is optionally substituted as described herein for heterocyclyl.

“Heteroaryl” refers to a 5- to 18-membered ring system radical containing at least one aromatic ring, having the carbon count of one to seventeen carbon atoms, and containing a total of one to ten heteroatoms independently selected from the group consisting of nitrogen, oxygen, and sulfur. The heteroaryl radical is a monocyclic, bicyclic, tricyclic, or tetracyclic ring system. The bicyclic, tricyclic, or tetracyclic heteroaryl radical is a fused and/or bridged ring system. An optionally substituted heteroaryl is a heteroaryl radical that is optionally substituted by one, two, three, four, or five substituents independently selected from the group consisting of alkyl, alkenyl, alkoxy, halo, haloalkyl, haloalkenyl, cyano, oxo, thioxo, nitro, oxo, aryl, aralkyl, cycloalkyl, heterocyclyl, heteroaryl, or heteroarylalkyl, —R″—OR′, —R″—OC(O)—R′, —R″—N(R′)₂, —R″—C(O)R′, —R″—C(O)OR′, —R″—C(O)N(R′)₂, —R″—N(R′)C(O)OR′″, —R″—N(R′)C(O)R′″, —R″—N(R′)S(O)ₜR″″ (where t is 1 or 2), —R″—S(O)ₜOR″ (where t is 1 or 2), —R″—S(O)ₜR″ (where p is 0, 1, or 2), and —R″—S(O)ₜN(R′)₂ (where t is 1 or 2), where each R′ is independently hydrogen, alkyl, alkenyl, haloalkyl, cycloalkyl, aryl, heterocyclyl, or heteroaryl; each R″ is independently a direct bond or a linear or branched alkylene or alkenylene chain; and each R′″ is alkyl, alkenyl, haloalkyl, cycloalkyl, aryl, heterocyclyl, or heteroaryl. The nitrogen, carbon, or sulfur atoms in the heterocyclyl radical may be optionally oxidized (when the substituent is oxo and is present on the heteroatom), provided that at least one ring in heteroaryl remains aromatic; the nitrogen atom may be optionally quaternized (when the substituent is alkyl, alkenyl, aryl, aralkyl, cycloalkyl, heterocyclyl, heteroaryl, —R″—OR′, —R″—OC(O)—R′, —R″—N(R′)₂, —R″—C(O)R′, —R″—C(O)OR′, —R″—C(O)N(R′)₂, —R″—N(R′)C(O)O′R″, —R″—N(R′)C(O)R″, —R″—N(R′)S(O)ₜR′″ (where t is 1 or 2), —R″—S(O)ₜOR′″ (where t is 1 or 2), —R″—S(O)ₚR′″ (where p is 0, 1, or 2), and —R″—S(O)ₜN(R′)₂ (where t is 1 or 2), where R″ is a linear or branched alkylene or alkenylene chain, and R′ and R′″ are as defined above), provided that at least one ring in heteroaryl remains aromatic. Examples of optionally substituted heteroaryl radicals include, but are not limited to, azepinyl, acridinyl, benzimidazolyl, benzthiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyl, naphthyridinyl, oxadiazolyl, 2-oxoazepinyl, oxazolyl, oxiranyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, pyrrolyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl and thiophenyl (i.e., thienyl).

The phrase “pharmaceutically acceptable” is employed herein to refer to those compounds, salts, compositions, dosage forms, etc., which are—within the scope of sound medical judgment—suitable for use in contact with the tissues of human beings and/or other mammals without excessive toxicity, irritation, allergic response, or other problem or complication, commensurate with a reasonable benefit/risk ratio. In some aspects, “pharmaceutically acceptable” means approved by a regulatory agency of the federal or a state government, or listed in the U.S. Pharmacopeia or other generally recognized pharmacopeia for use in mammals (e.g., animals), and more particularly, in humans.

“Prodrugs” are compounds that after administration are metabolized or otherwise chemically transformed into an active moiety. Prodrugs may be derivatives of an active compound. Prodrugs may or may not be active prior to conversion into an active form in vivo.

The term “treating” is used herein, for instance, in reference, for example, to methods of treating inflammatory diseases or to a gastrointestinal disease, and generally includes the administration of a compound or composition which reduces the frequency of, or delays the onset of, symptoms of a medical condition (e.g., autoimmune disease, inflammatory disorder, gastrointestinal disorder) in a subject relative to a subject not receiving the compound or composition. This can include reversing, reducing, or arresting the symptoms, clinical signs, and underlying pathology of a condition in a manner to improve or stabilize a subject's condition (e.g., regression of symptoms of an autoimmune or inflammatory disease such as improvement in the MAYO score in the treatment of ulcerative colitis).

The embodiments disclosed herein encompass all pharmaceutically acceptable compounds of the compound of (I)-(IL) being isotopically-labelled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. These radiolabeled compounds could be useful to help determine or measure the effectiveness of the compounds, by characterizing, for example, the site or mode of action, or binding affinity to pharmacologically important site of action. Certain isotopically-labelled compounds of (I)-(IL), for example, those incorporating a radioactive isotope, are useful in drug and/or substrate tissue distribution studies. The radioactive isotopes tritium, i.e., ³H, and carbon-14, i.e., ¹⁴C, are particularly useful for this purpose in view of their case of incorporation and ready means of detection.

Substitution with heavier isotopes such as deuterium, i.e., ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of (I)-(IL) can generally be prepared by conventional techniques known to those skilled in the art or by processes analogous to those described in the Preparations and Examples as set out below using an appropriate isotopically-labeled reagent in place of the non-labeled reagent previously employed.

The embodiments disclosed herein encompass the in vivo metabolic products of the disclosed compounds. Such products may result from, for example, the oxidation, reduction, hydrolysis, amidation, esterification, and the like of the administered compound, primarily due to enzymatic processes. Accordingly, the disclosure includes compounds produced by a process comprising administering a compound of this disclosure to a mammal for a period of time sufficient to yield a metabolic product thereof. Such products are typically identified by administering a radiolabelled compound of the disclosure in a detectable dose to an animal, such as rat, mouse, guinea pig, monkey, or to human, allowing sufficient time for metabolism to occur, and isolating its conversion products from the urine, blood or other biological samples.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid (TFA), undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2 dimethylaminoethanol, 2 diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N ethylpiperidine, polyamine resins and the like. Particularly preferred organic bases are isopropylamine, diethylamine, ethanolamine, trimethylamine, dicyclohexylamine, choline and caffeine.

A “pharmaceutical composition” refers to a formulation of a compound of the disclosure and a medium generally accepted in the art for the delivery of the biologically active compound to mammals, e.g., humans. Such a medium includes all pharmaceutically acceptable carriers, diluents and excipients therefor.

“Effective amount” or “therapeutically effective amount” refers to that amount of a compound of the disclosure which, when administered to a mammal, preferably a human, is sufficient to effect treatment in the mammal, preferably a human. The amount of a compound which constitutes a “therapeutically effective amount” will vary depending on the compound, the condition and its severity, the manner of administration, and the age of the mammal to be treated, but can be determined routinely by one of ordinary skill in the art having regard to his own knowledge and to this disclosure.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are nonsuperimposeable mirror images of one another. The present disclosure also contemplates “diastereomers”, which refers to non-mirror image of non-identical stereoisomers. Diastereomers occur when two or more stereoisomers of a compound have different configurations at one or more of the equivalent stereocenters and are not mirror images of each other.

A “tautomer” refers to a proton shift from one atom of a molecule to another atom of the same molecule. The present disclosure includes tautomers of any said compounds.

“Abnormal cell growth”, as used herein, unless otherwise indicated, means cell growth that is independent of normal regulatory mechanisms (e.g., loss of contact inhibition). Abnormal cell growth may be benign (not cancerous) or malignant (cancerous).

By reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by reserving the right to proviso out or exclude any individual substituents, analogs, compounds, ligands, structures, or groups thereof, or any members of a claimed group, less than the full measure of this disclosure can be claimed for any reason.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

### II. Compounds

The compounds described herein are compounds for FGFRs inhibitors. In one embodiment, a compound having a structure of Formula (I) is provided:

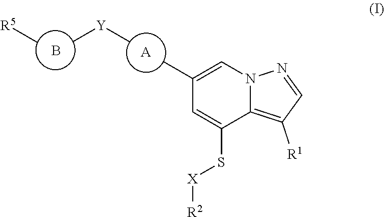

or stereoisomer, salt, or tautomer thereof, wherein R¹ is —CN, —Cl, —Br, —CF₂H, or —CF₃; R² is C₁-C₆ alkyl, C₁-C₆ heteroalkyl, aryl, heteroaryl, a fused bicyclic, C₃-C₈ cycloalkyl, C₃-C₈ heterocycloalkyl, or —NR³R⁴; R³ and R⁴ are, each independently, H or C₁-C₄ alkyl; R⁵—H, —OH, —CN, —F, C₁-C₆ alkyl, C₃-C₈ cycloalkyl, C₁-C₆ heteroalkyl, a 5-6 membered heteroaryl, C₃-C₈ heterocycloalkyl, —C(═O)R⁶, —OC(═O)R⁶, —CH₂C(═O)OR⁶, —NR⁷R⁸, —NR⁷C(═O)R⁶, —NRC(═O)OR⁶, —CH₂C(═O)NR⁷N⁸, —NR⁷R⁸CH₂C(═O)NR⁷N⁸, —CH₂NR⁷C(═O)R⁶, sulfonylmethane, oxo, phosphate, or a combination thereof; wherein the C₁-C₆ alkyl or C₁-C₆ heteroalkyl of R⁵ is optionally substituted with

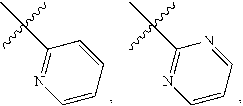 [SMILES: *c1ccccn1 ; *c1ncccn1]

or —OH; R⁶ is a hydrogen, C₁-C₄ alkyl, C₁-C₅ heteroalkyl, or C₄-C₆ heterocycloalkyl, wherein the C₄-C₆ heterocycloalkyl is substituted with

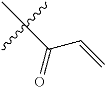 [SMILES: *C(=O)C=C]

or halo; R⁷ and R⁸ are, each independently, H, C₁-C₆ alkyl, C₁-C₆ alkyl alcohol, sulfonylmethane, C₃-C₆ cycloalkyl, or 5-6 membered heteroaryl, wherein the C₁-C₆ alkyl or C₃-C₆ cycloalkyl is optionally substituted with fluoro, C₃-C₆ cycloalkyl, or 5-6 membered heteroaryl; X is —(CH₂)n-, —(CH₂)nO—, —(CHCH₃)n-, —(CHCH₃)n-(CH₂)m-, —(CHCH₃)n-(CH₂)mO—, or a direct bond; Y is —CH₂—, —CHCH₃—, —C(CH₃)₂—, —(CH₂)ₚC(═O)—, —NCH₃—, or a direct bond; n is an integer between 1 and 4; m is an integer between 1 and 4; p is an integer between 0 and 4; A is an aryl, a 5-6 membered heteroaryl, a halo, or a fused heterocyclic; and B is C₃-C₈ cycloalkyl, C₃-C₈ heterocycloalkyl, a fused bicyclic, a bridged bicyclic, a spirocyclic, or absent.

In one embodiment, R¹ is —CN, —Cl, —Br, —CF₂H, or —CF₃. In some embodiments, R¹ has one of the following structures:

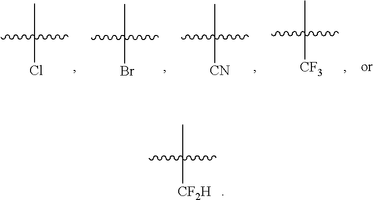

In some embodiments, R¹ is

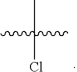 [SMILES: *Cl]

In some embodiments, R¹ is

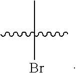 [SMILES: *Br]

In some embodiments, R¹ is

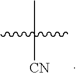 [SMILES: *C#N]

In some embodiments, R¹ is

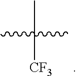 [SMILES: *C(F)(F)F]

In some embodiments, R¹ is

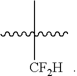 [SMILES: *C(F)F]

In one embodiment, the compound has the following structure of Formula (IA):

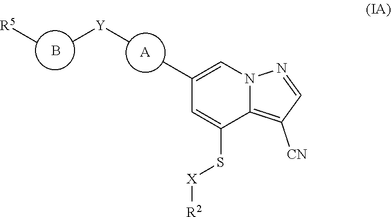

or a stereoisomer, salt or tautomer thereof.

In one embodiment, X is —(CH₂)n-, —(CH₂)nO—, —(CHCH₃)n-, —(CHCH₃)n-(CH₂)m-, —(CHCH₃)n-(CH₂)mO—, or a direct bond. In some embodiments, X is —(CH₂)n-. In some embodiments, X is —(CH₂)nO—. In some embodiments, X is —(CHCH₃)n-. In some embodiments, X is —(CHCH₃)n-(CH₂)m-. In some embodiments, X is —(CHCH₃)n-(CH₂)mO—. In some embodiments, X is a direct bond.

In one embodiment, n is an integer between 1 and 4. In some embodiments, n is an integer between 1 and 2. In some embodiments, n is an integer of 1 or 2. In some embodiments, n is an integer of 1. In some embodiments, n is an integer of 2.

In one embodiment, m is an integer between 1 and 4. In some embodiments, m is an integer between 1 and 2. In some embodiments, m is an integer of 1 or 2. In some embodiments, m is an integer of 1. In some embodiments, m is an integer of 2.

In one embodiment, the compound has the following structure of Formula (IB):

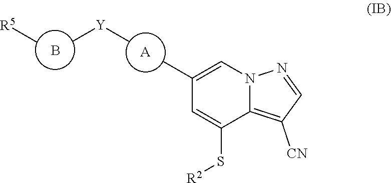

or a stereoisomer, salt or tautomer thereof.

In some embodiments, A is an aryl, a 5-6 membered heteroaryl, a halo, or a fused heterocyclic.

In some embodiments, A is an aryl. In some embodiments, A is an aryl. In some certain embodiments, the aryl of A is a phenyl.

In some embodiments, A is a halo. In some certain embodiments, the halo of A is —F, —Cl, or —Br. In some embodiments, A is —F. In some embodiments, A is —Cl. In some embodiments, A is —Br.

In some embodiments, A is a fused heterocyclic. In some certain embodiments, the fused heterocyclic of A is indole, isoindole, benzimidazole, purine, indazole, benzoxazole, benzisooxazole, quinoline, isoquinoline, quinoxaline, quinazoline, cinnoline, phtalazine, 5H-pyrrolo[2,3-b]pyrazine, or 4,5,6,7-tetrahydropyrazolo[1,5-a]pyrazine. In some embodiments, the fused heterocyclic of A is indole. In some embodiments, the fused heterocyclic of A is isoindole. In some embodiments, the fused heterocyclic of A is benzimidazole. In some embodiments, the fused heterocyclic of A is purine. In some embodiments, the fused heterocyclic of A is indazole. In some embodiments, the fused heterocyclic of A is benzoxazole. In some embodiments, the fused heterocyclic of A is benzisooxazole. In some embodiments, the fused heterocyclic of A is quinoline. In some embodiments, the fused heterocyclic of A is isoquinoline. In some embodiments, the fused heterocyclic of A is quinoxaline. In some embodiments, the fused heterocyclic of A is quinazoline. In some embodiments, the fused heterocyclic of A is cinnoline. In some embodiments, the fused heterocyclic of A is phtalazine. In some embodiments, the fused heterocyclic of A is 5H-pyrrolo[2,3-b]pyrazine. In some embodiments, the fused heterocyclic of A is 4,5,6,7-tetrahydropyrazolo[1,5-a]pyrazine. In some embodiments, the fused heterocyclic of A is 5H-pyrrolo[2,3-b]pyrazine or 4,5,6,7-tetrahydropyrazolo[1,5-a]pyrazine.

In some embodiments, A is a 5-6 membered heteroaryl. In some embodiments, the 5-6 membered heteroaryl of A is a 5 membered heteroaryl. In certain embodiments, the 5 membered heteroaryl is pyrazole or triazole. In some embodiments, the 5-6 membered heteroaryl of A is a 6 membered heteroaryl. In certain embodiments, the 6 membered heteroaryl is pyridine. In some embodiments, the 5-6 membered heteroaryl of A is optionally substituted with C₁-C₄ alkyl, C₁-C₄ heteroalkyl, or halo. In some embodiments, the 5-6 membered heteroaryl of A is optionally substituted with methyl (C₁ alkyl), cyano (C₁ heteroalkyl), methoxylethyl (C₃ heteroalkyl), fluoro (halo), or chloro (halo). In some certain embodiments, the 5-6 membered heteroaryl is 5-methylpyrazole.

In some embodiments, the aryl, 5-6 membered heteroaryl, or fused heterocyclic of A is mono or di-substituted with —CH₃, —F, —Cl, —CN, —NH₂, —CH₂OCH₃, or combination thereof. In some embodiments, the aryl, 5-6 membered heteroaryl, or fused heterocyclic of A is mono substituted with —CH₃, —F, —Cl, —CN, —NH₂, or —CH₂OCH₃. In some embodiments, the aryl, 5-6 membered heteroaryl, or fused heterocyclic of A is disubstituted with —CH₃, —F, —Cl, —CN, —NH₂, —CH₂OCH₃, or combination thereof.

In one embodiment, A has one of the following structures:

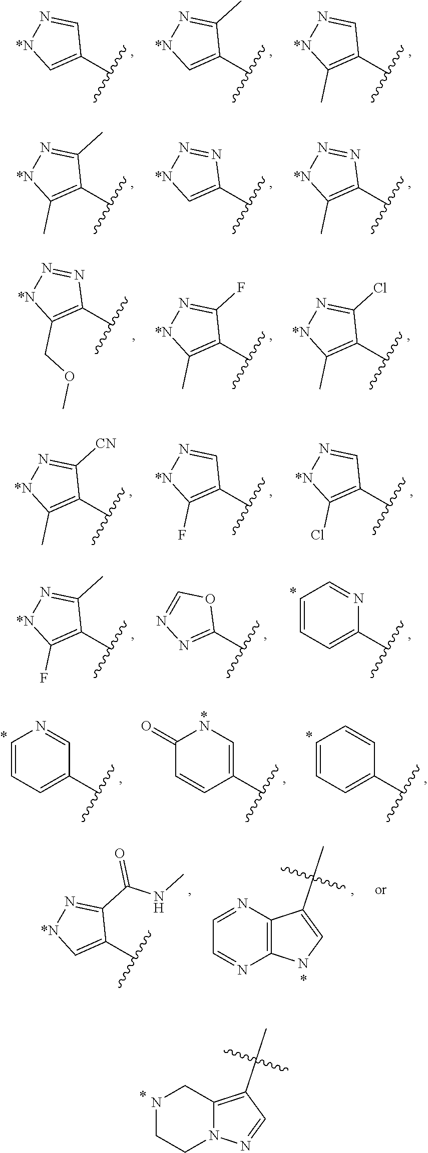

wherein * indicates a location of a bond to Y. In some embodiments, A is

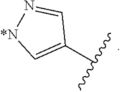

In some embodiments, A is

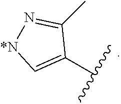

In some embodiments, A is

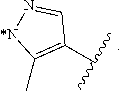

In some embodiments, A is

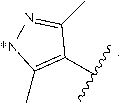

In some embodiments, A is

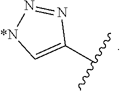

In some embodiments, A is

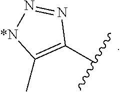

In some embodiments, A is

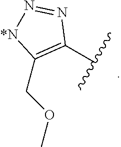

In some embodiments, A is

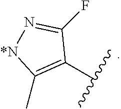

In some embodiments, A is

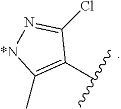

In some embodiments, A is

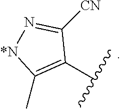

In some embodiments, A is

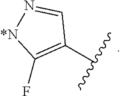

In some embodiments, A is

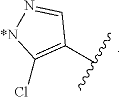

In some embodiments, A is

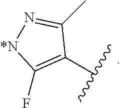

In some embodiments, A is

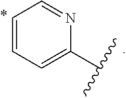 [SMILES: *c1ccccn1]

In some embodiments, A is

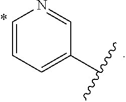 [SMILES: *c1cccnc1]

In some embodiments, A is

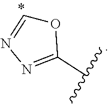 [SMILES: *c1nnco1]

In some embodiments, A is

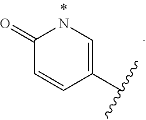 [SMILES: *c1ccc(=O)[nH]c1]

In some embodiments, A is

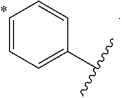 [SMILES: *c1ccccc1]

In some embodiments, A is

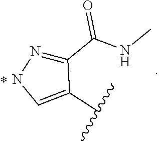 [SMILES: *c1c[nH]nc1C(=O)NC]

In some embodiments, A is

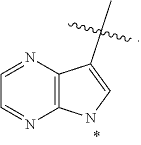 [SMILES: *c1c[nH]c2nccnc12]

In some embodiments, A is

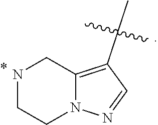 [SMILES: *c1cnn2c1CNCC2]

In some embodiments, A has one of the following structures:

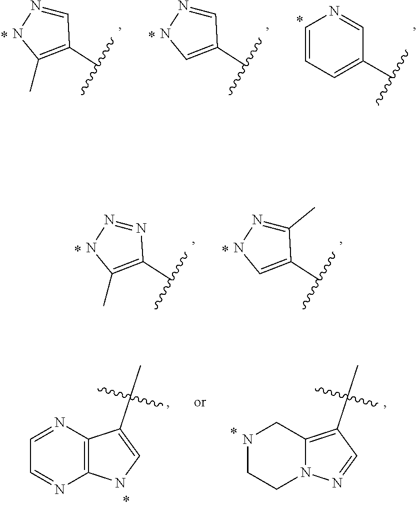

wherein * indicates a location of a bond to Y. In some embodiments, A has one of the following structures:

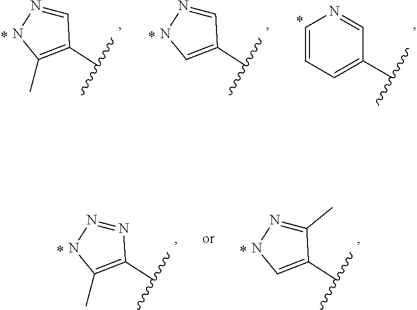

wherein * indicates a location of a bond to Y.

In one embodiment, the compound has one of the following structures of Formula (C)-(IG):

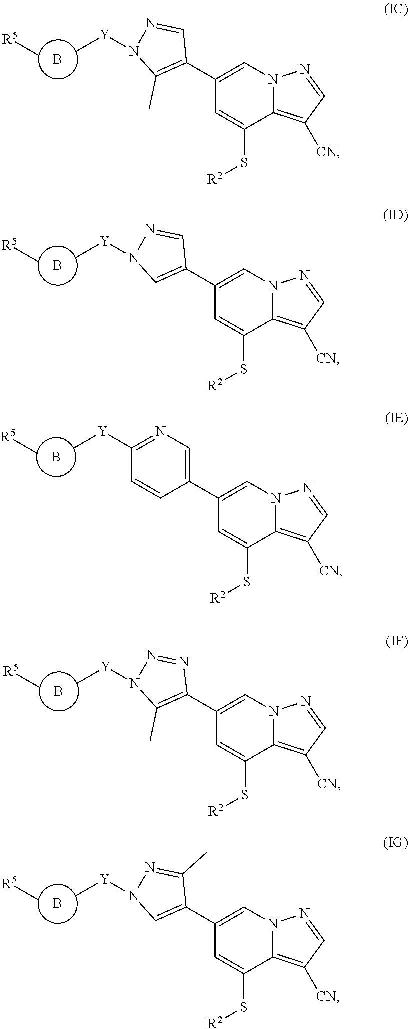

or a stereoisomer, salt or tautomer thereof.

In one embodiment, Y is —CH₂—, —CHCH₃—, —C(CH₃)₂—, —(CH₂)ₚC(═O)—, —NCH₃—, or a direct bond. In some embodiments, Y is —CH₂—. In some embodiments, Y is —CHCH₃—. In some embodiments, Y is C(CH₃)₂—. In some embodiments, Y is a direct bond. In some embodiments, Y is —(CH₂)ₚC(═O)—. In some embodiments, Y is —(CH₂)ₚC(═O)—, wherein p is an integer between 0 and 2. In some embodiments, Y is —NCH₃—.

In one embodiment, p is an integer between 0 and 4. In some embodiments, p is an integer of 0. In some embodiments, p is an integer of 1. In some embodiments, p is an integer of 2. In some embodiments, p is an integer of 3. In some embodiments, p is an integer of 4. In some embodiments, p is an integer between 0 and 2.

In one embodiment, the compound has one of the following structures of Formula (IH)-(IL):

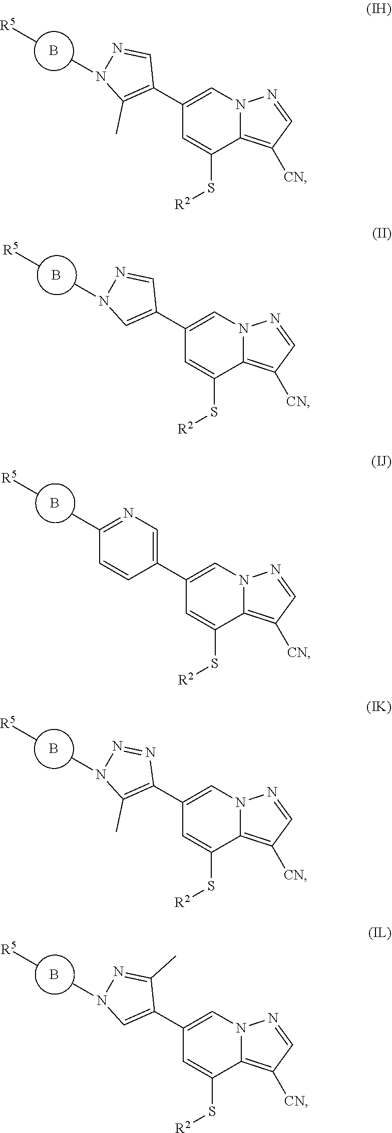

or a stereoisomer, salt or tautomer thereof.

In one embodiment, R² is C₁-C₆ alkyl, C₁-C₆ heteroalkyl, aryl, heteroaryl, a fused bicyclic, C₃-C₈ cycloalkyl, C₃-C₈ heterocycloalkyl, or —NR³R⁴. In some embodiments, the aryl, heteroaryl, or fused bicyclic of R² is further substituted with fluoro, chloro, cyano, methyl, amine, —CF₂H, —CF₃, C₁-C₃ alkylalcohol, C₁-C₃ alkoxyl, or a combination thereof. In some embodiments, the aryl, heteroaryl, or fused bicyclic of R² is mono-, di-, or tri-substituted with fluoro, chloro, cyano, methyl, amine, —CF₂H, —CF₃, C₁-C₃ alkylalcohol, C₁-C₃ alkoxyl, or a combination thereof. In some embodiments, for example, the aryl, heteroaryl, or fused bicyclic of R² is substituted with two fluoro substituents. In some embodiments, for example, the aryl, heteroaryl, or fused bicyclic of R² is substituted with two fluoro substituents and one methyl substituent. In some embodiments, for example, the aryl, heteroaryl, or fused bicyclic of R² is substituted with one fluoro substituent and one cyano substituent. In some embodiments, for example, the aryl, heteroaryl, or fused bicyclic of R² is substituted with one methyl, one cyano, and one fluoro substituents. In some embodiments, for example, the aryl, heteroaryl, or fused bicyclic of R² is substituted with one fluoro substituent and one methyl substituent. In some embodiments, for example, the aryl, heteroaryl, or fused bicyclic of R² is substituted with one chloro substituent and one cyano substituent. In some embodiments, for example, the aryl, heteroaryl, or fused bicyclic of R² is substituted with two methyl substituents. In some embodiments, for example, the aryl, heteroaryl, or fused bicyclic of R² is substituted with one fluoro substituent and one amino substituent. In some embodiments, for example, the aryl, heteroaryl, or fused bicyclic of R² is substituted with one fluoro substituent and one deuterium substituent. In some embodiments, for example, the aryl, heteroaryl, or fused bicyclic of R² is substituted with one fluoro substituent and one methoxy substituent. In some embodiments, for example, the aryl, heteroaryl, or fused bicyclic of R² is substituted with two fluoro substituents and one methoxy substituent. In some embodiments, for example, the aryl, heteroaryl, or fused bicyclic of R² is substituted with one fluoro, one chloro, and one methyl substituents. In some embodiments, for example, the aryl, heteroaryl, or fused bicyclic of R² is substituted with two methyl and one fluoro substituents. In some embodiments, for example, the aryl, heteroaryl, or fused bicyclic of R² is substituted with one fluoro, one methoxyl, and one methyl substituents. In some embodiments, for example, the aryl, heteroaryl, or fused bicyclic of R² is substituted with one cyano substituent and one methoxy substituent. In some embodiments, for example, the aryl, heteroaryl, or fused bicyclic of R² is substituted with one cyano, one methoxyl, and one methyl substituents.

In one embodiment, the fused bicyclic of R² has one of the following structures:

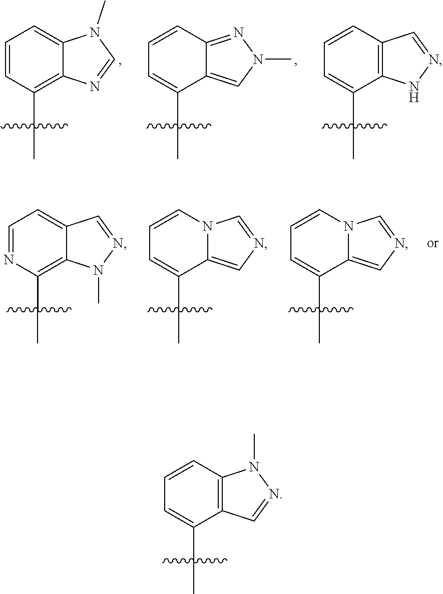

In some embodiments, the fused bicyclic of R² has one of the following structures:

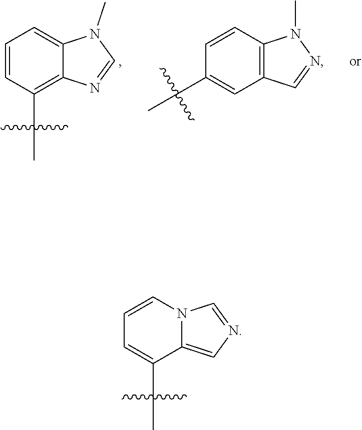 [SMILES: *c1cccc2c1ncn2C ; *c1ccc2c(cnn2C)c1 ; *c1cccn2cncc12]

In some embodiments, the fused bicyclic of R² is

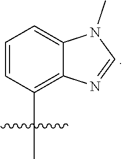 [SMILES: *c1cccc2c1ncn2C]

In some embodiments, the fused bicyclic of R² is

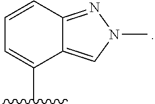 [SMILES: *c1cccc2nn(C)cc12]

In some embodiments, the fused bicyclic of R² is

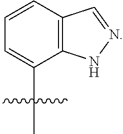 [SMILES: *c1cccc2cn[nH]c12]

In some embodiments, the fused bicyclic of R² is

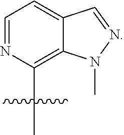 [SMILES: *c1nccc2cnn(C)c12]

In some embodiments, the fused bicyclic of R² is

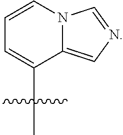 [SMILES: *c1cccn2cncc12]

In some embodiments, the fused bicyclic of R² is

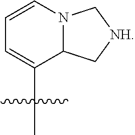 [SMILES: *C1=CC=CN2CNCC12]

In some embodiments, the fused bicyclic of R² is

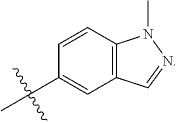 [SMILES: *c1ccc2c(cnn2C)c1]

In one embodiment, the aryl of R² has one of the following structures:

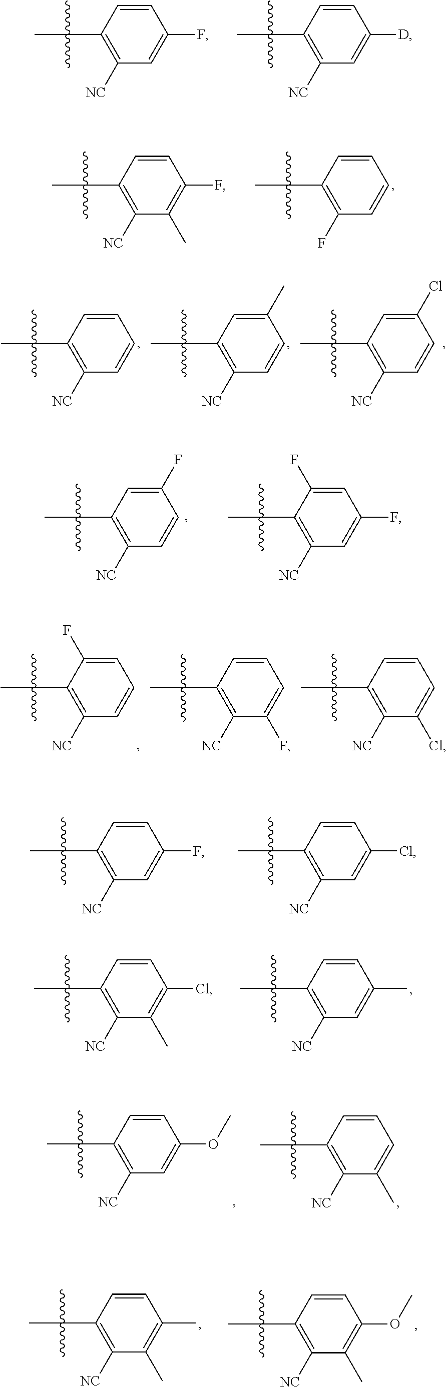

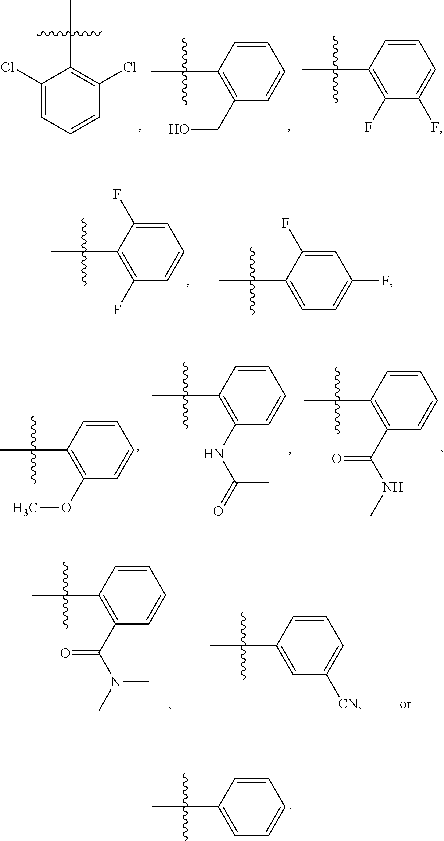

In some embodiments, the aryl of R² is

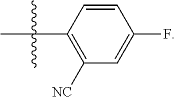 [SMILES: *c1ccc(F)cc1C#N]

In some embodiments, the aryl of R² is

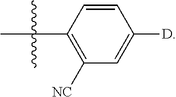

In some embodiments, the aryl of R² is

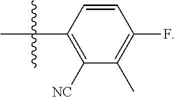 [SMILES: *c1ccc(F)c(C)c1C#N]

In some embodiments, the aryl of R² is

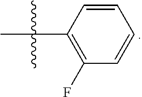 [SMILES: *c1ccccc1F]

In some embodiments, the aryl of R² is

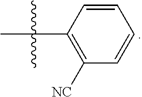 [SMILES: *c1ccccc1C#N]

In some embodiments, the aryl of R² is

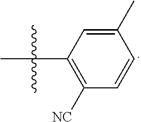 [SMILES: *c1cc(C)ccc1C#N]

In some embodiments, the aryl of R² is

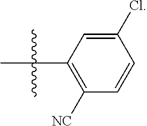 [SMILES: *c1cc(Cl)ccc1C#N]

In some

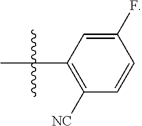 [SMILES: *c1cc(F)ccc1C#N]

embodiments, the aryl of R² is

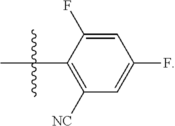 [SMILES: *c1c(F)cc(F)cc1C#N]

In some embodiments, the aryl of R² is

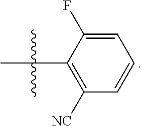 [SMILES: *c1c(F)cccc1C#N]

In some, embodiments, the aryl of R² is

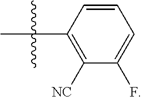 [SMILES: *c1cccc(F)c1C#N]

In some embodiments, the aryl of R² is

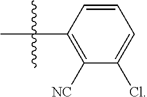 [SMILES: *c1cccc(Cl)c1C#N]

In some embodiments, the aryl of R² is

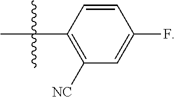 [SMILES: *c1ccc(F)cc1C#N]

In some embodiments, the aryl of R² is

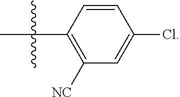 [SMILES: *c1ccc(Cl)cc1C#N]

In some embodiments, the aryl of R² is

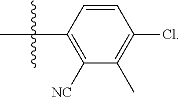 [SMILES: *c1ccc(Cl)c(C)c1C#N]

In some embodiments, the aryl of R² is

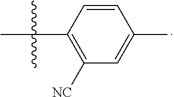 [SMILES: *c1ccc(C)cc1C#N]

In some embodiments, the aryl of R² is

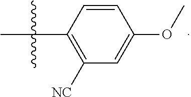 [SMILES: *c1ccc(OC)cc1C#N]

In some embodiments, the aryl of R² is

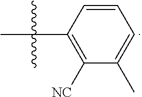 [SMILES: *c1cccc(C)c1C#N]

In some embodiments, the aryl of R² is

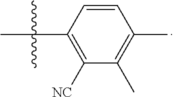 [SMILES: *c1ccc(C)c(C)c1C#N]

In some embodiments, the aryl of R² is

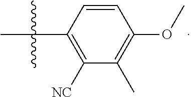 [SMILES: *c1ccc(OC)c(C)c1C#N]

In some embodiments, the aryl of R² is

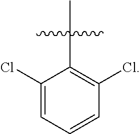 [SMILES: *c1c(Cl)cccc1Cl]

In some embodiments, the aryl of R² is

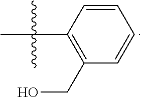 [SMILES: *c1ccccc1CO]

In some embodiments, the aryl of R² is

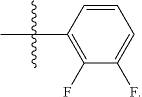 [SMILES: *c1cccc(F)c1F]

In some embodiments, the aryl of R² is

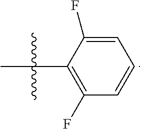 [SMILES: *c1c(F)cccc1F]

In some embodiments, the aryl of R² is

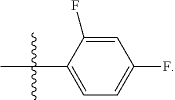 [SMILES: *c1ccc(F)cc1F]

In some embodiments, the aryl of R² is

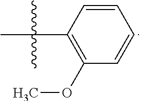 [SMILES: *c1ccccc1OC]

In some embodiments, the aryl of R² is

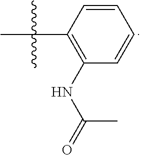 [SMILES: *c1ccccc1NC(C)=O]

In some embodiments, the aryl of R² is

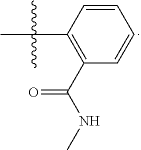 [SMILES: *c1ccccc1C(=O)NC]

In some embodiments, the aryl of R² is

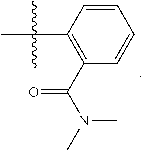 [SMILES: *c1ccccc1C(=O)N(C)C]

In some embodiments, the aryl of R² is

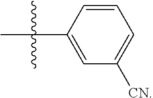 [SMILES: *c1cccc(C#N)c1]

In some embodiments, the aryl of R² is

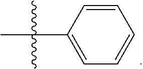 [SMILES: *c1ccccc1]

In one embodiment, the heteroaryl of R² has one of the following structures:

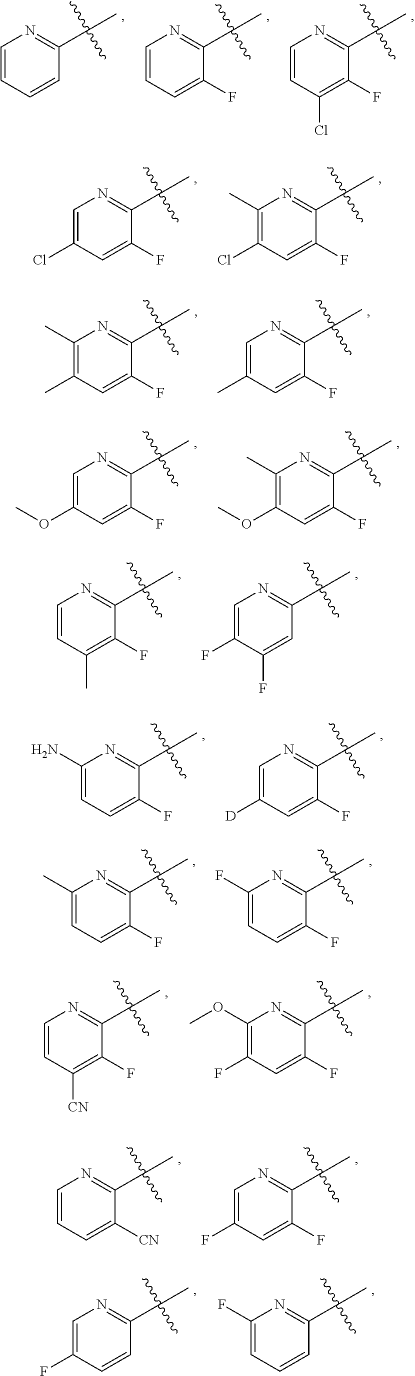

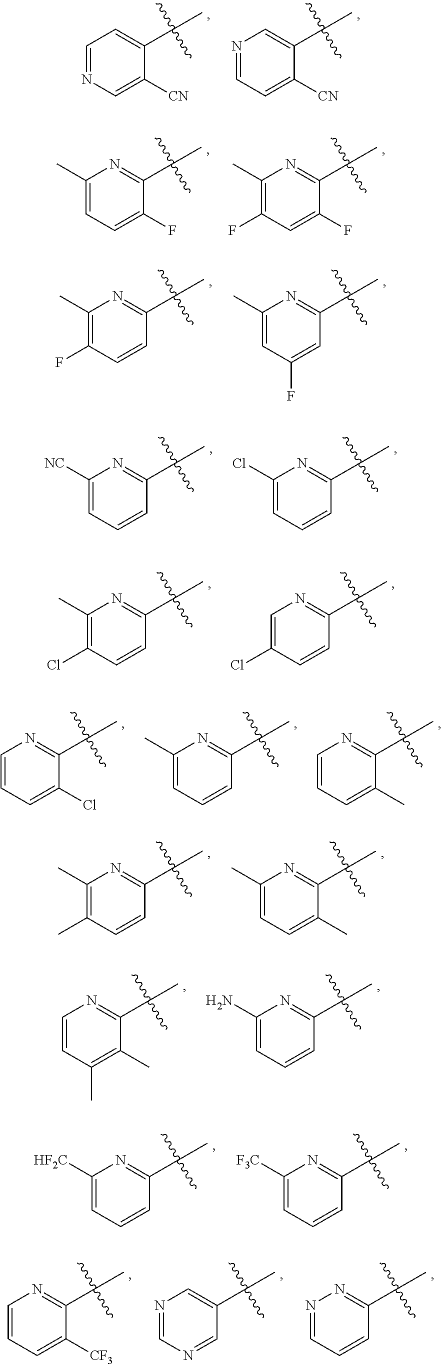

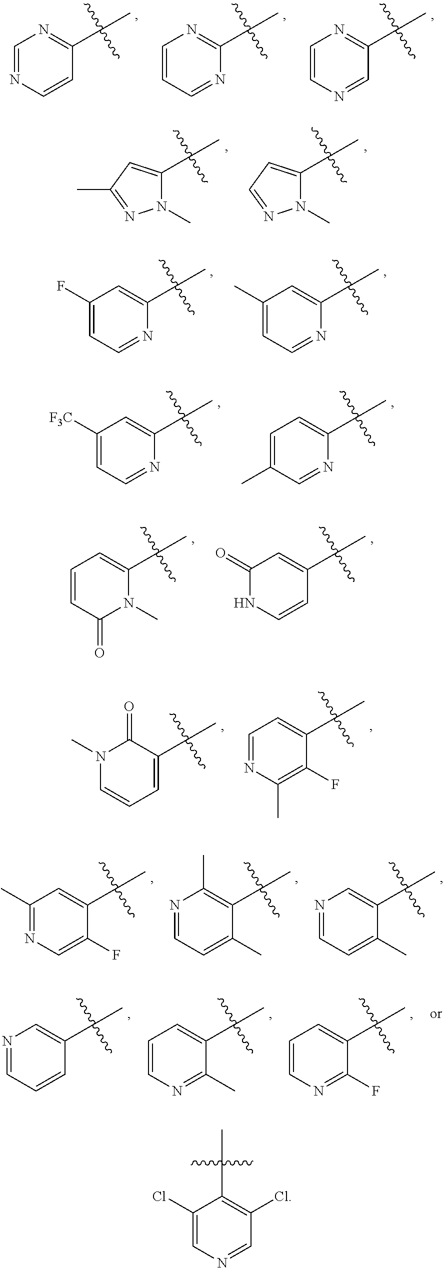

In some embodiments, the heteroaryl of R² is

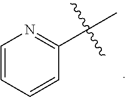 [SMILES: *c1ccccn1]

In some embodiments, the heteroaryl of R² is

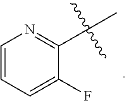 [SMILES: *c1ncccc1F]

In some embodiments, the heteroaryl of R² is

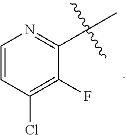 [SMILES: *c1nccc(Cl)c1F]

In some embodiments, the heteroaryl of R² is

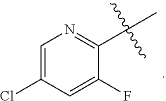 [SMILES: *c1ncc(Cl)cc1F]

In some embodiments, the heteroaryl of R² is

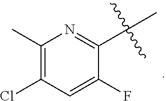 [SMILES: *c1nc(C)c(Cl)cc1F]

In some embodiments, the heteroaryl of R² is

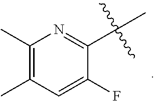 [SMILES: *c1nc(C)c(C)cc1F]

In some embodiments, the heteroaryl of R² is

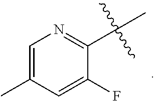 [SMILES: *c1ncc(C)cc1F]

In some embodiments, the heteroaryl of R² is

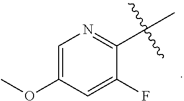 [SMILES: *c1ncc(OC)cc1F]

In some embodiments, the heteroaryl of R² is

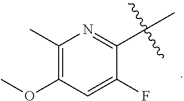 [SMILES: *c1nc(C)c(OC)cc1F]

In some embodiments, the heteroaryl of R² is

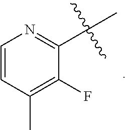 [SMILES: *c1nccc(C)c1F]

In some embodiments, the heteroaryl of R² is

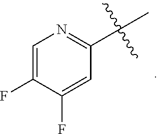 [SMILES: *c1cc(F)c(F)cn1]

In some embodiments, the heteroaryl of R² is

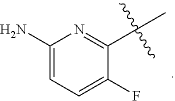 [SMILES: *c1nc(N)ccc1F]

In some embodiments, the heteroaryl of R² is

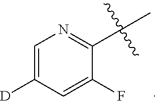 [SMILES: *c1ncc([2H])cc1F]

In some embodiments, the heteroaryl of R² is

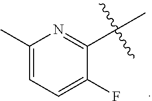 [SMILES: *c1nc(C)ccc1F]

In some embodiments, the heteroaryl of R² is

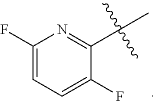 [SMILES: *c1nc(F)ccc1F]

In some embodiments, the heteroaryl of R² is

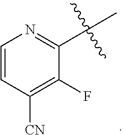 [SMILES: *c1nccc(C#N)c1F]

In some embodiments, the heteroaryl of R² is

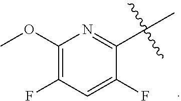 [SMILES: *c1nc(OC)c(F)cc1F]

In some embodiments, the heteroaryl of R² is

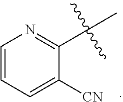 [SMILES: *c1ncccc1C#N]

In some embodiments, the heteroaryl of R² is

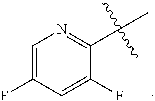 [SMILES: *c1ncc(F)cc1F]

In some embodiments, the heteroaryl of R² is

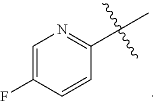 [SMILES: *c1ccc(F)cn1]

In some embodiments, the heteroaryl of R² is

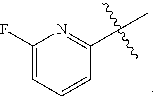 [SMILES: *c1cccc(F)n1]

In some embodiments, the heteroaryl of R² is

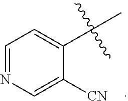 [SMILES: *c1ccncc1C#N]

In some embodiments, the heteroaryl of R² is

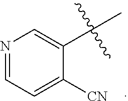 [SMILES: *c1cnccc1C#N]

In some embodiments, the heteroaryl of R² is

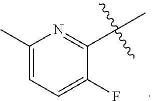 [SMILES: *c1nc(C)ccc1F]

In some embodiments, the heteroaryl of R² is

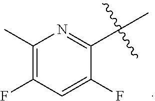 [SMILES: *c1nc(C)c(F)cc1F]

In some embodiments, the heteroaryl of R² is

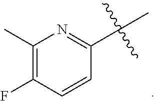 [SMILES: *c1ccc(F)c(C)n1]

In some embodiments, the heteroaryl of R² is

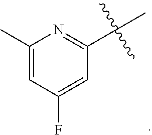 [SMILES: *c1cc(F)cc(C)n1]

In some embodiments, the heteroaryl of R² is

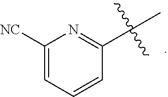 [SMILES: *c1cccc(C#N)n1]

In some embodiments, the heteroaryl of R² is

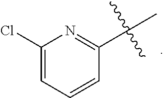 [SMILES: *c1cccc(Cl)n1]

In some embodiments, the heteroaryl of R² is

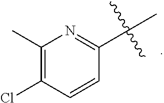 [SMILES: *c1ccc(Cl)c(C)n1]

In some embodiments, the heteroaryl of R² is

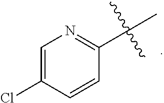 [SMILES: *c1ccc(Cl)cn1]

In some embodiments, the heteroaryl of R² is

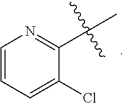 [SMILES: *c1ncccc1Cl]

In some embodiments, the heteroaryl of R² is

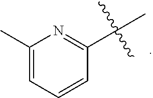 [SMILES: *c1cccc(C)n1]

In some embodiments, the heteroaryl of R² is

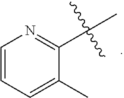 [SMILES: *c1ncccc1C]

In some embodiments, the heteroaryl of R² is

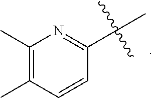 [SMILES: *c1ccc(C)c(C)n1]

In some embodiments, the heteroaryl of R² is

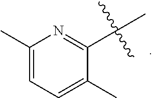

In some embodiments, the heteroaryl of R² is

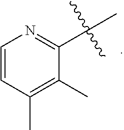 [SMILES: *c1nccc(C)c1C]

In some embodiments, the heteroaryl of R² is

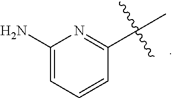 [SMILES: *c1cccc(N)n1]

In some embodiments, the heteroaryl of R² is

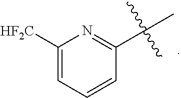

In some embodiments, the heteroaryl of R² is

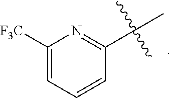 [SMILES: *c1cccc(C(F)(F)F)n1]

In some embodiments, the heteroaryl of R² is

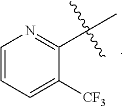 [SMILES: *c1ncccc1C(F)(F)F]

In some embodiments, the heteroaryl of R² is

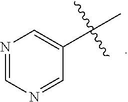 [SMILES: *c1cncnc1]

In some embodiments, the heteroaryl of R² is

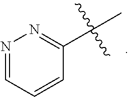 [SMILES: *c1cccnn1]

In some embodiments, the heteroaryl of R² is

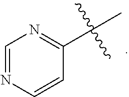 [SMILES: *c1ccncn1]

In some embodiments, the heteroaryl of R² is

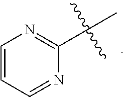 [SMILES: *c1ncccn1]

In some embodiments, the heteroaryl of R² is

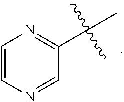 [SMILES: *c1cnccn1]

In some embodiments, the heteroaryl of R² is

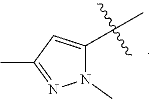 [SMILES: *c1cc(C)nn1C]

In some embodiments, the heteroaryl of R² is

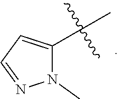 [SMILES: *c1ccnn1C]

In some embodiments, the heteroaryl of R² is

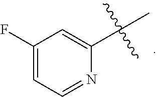 [SMILES: *c1cc(F)ccn1]

In some embodiments, the heteroaryl of R² is

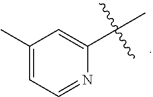 [SMILES: *c1cc(C)ccn1]

In some embodiments, the heteroaryl of R² is

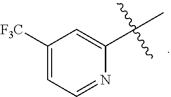 [SMILES: *c1cc(C(F)(F)F)ccn1]

In some embodiments, the heteroaryl of R² is

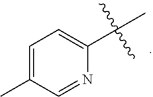 [SMILES: *c1ccc(C)cn1]

In some embodiments, the heteroaryl of R² is

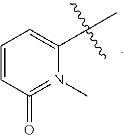

In some embodiments, the heteroaryl of R² is

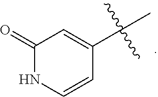 [SMILES: *c1cc[nH]c(=O)c1]

In some embodiments, the heteroaryl of R² is

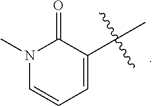 [SMILES: *c1cccn(C)c1=O]

In some embodiments, the heteroaryl of R² is

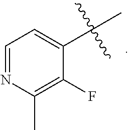 [SMILES: *c1ccnc(C)c1F]

In some embodiments, the heteroaryl of R² is

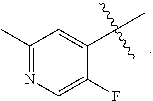 [SMILES: *c1cc(C)ncc1F]

In some embodiments, the heteroaryl of R² is

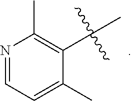 [SMILES: *c1c(C)ccnc1C]

In some embodiments, the heteroaryl of R² is

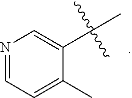 [SMILES: *c1cnccc1C]

In some embodiments, the heteroaryl of R² is

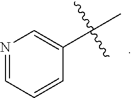 [SMILES: *c1cccnc1]

In some embodiments, the heteroaryl of R² is

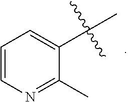 [SMILES: *c1cccnc1C]

In some embodiments, the heteroaryl of R² is

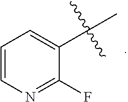 [SMILES: *c1cccnc1F]

In some embodiments, the heteroaryl of R² is

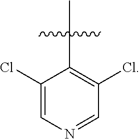 [SMILES: *c1c(Cl)cncc1Cl]

In one embodiment, the C₃-C₈ cycloalkyl or C₃-C₈ heterocycloalkyl of R² has one of the following structures:

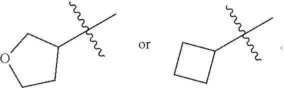 [SMILES: *C1CCOC1 ; *C1CCC1]

In some embodiments, the C₃-C₈ cycloalkyl or C₃-C₈ heterocycloalkyl of R² is

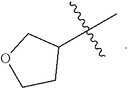 [SMILES: *C1CCOC1]

In some embodiments, the C₃-C₈ cycloalkyl or C₃-C₈ heterocycloalkyl of R² is

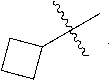 [SMILES: *C1CCC1]

In one embodiment, R² has one of the following structures:

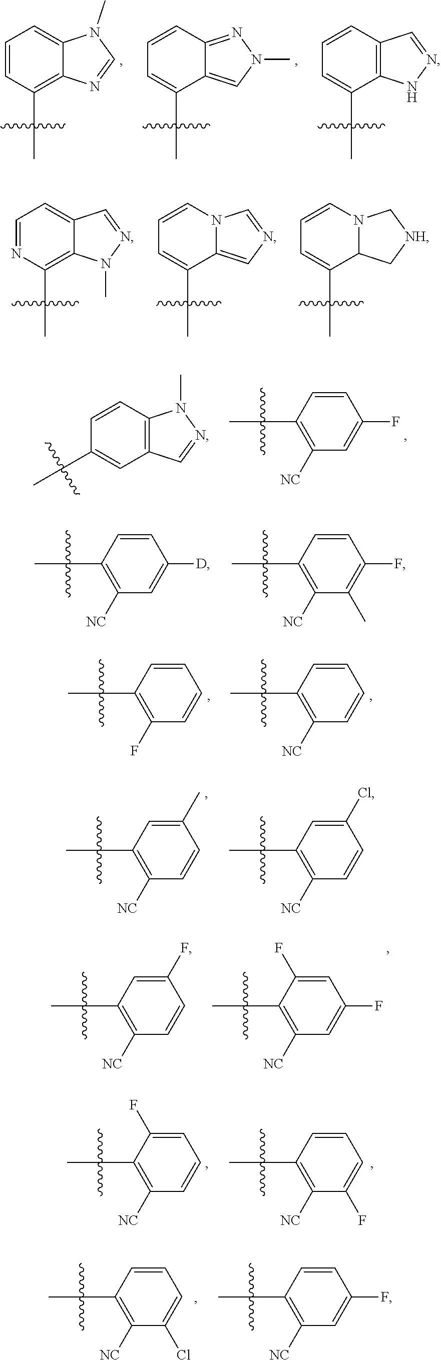

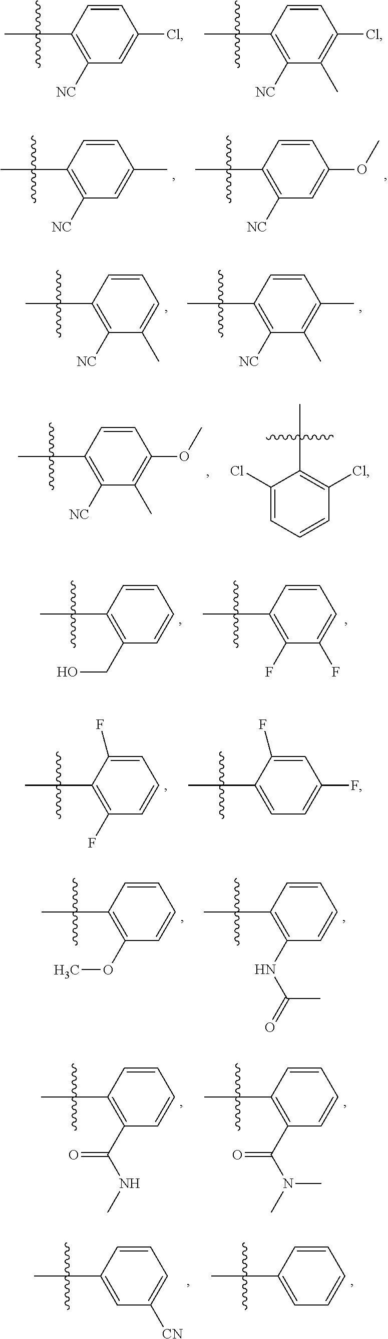

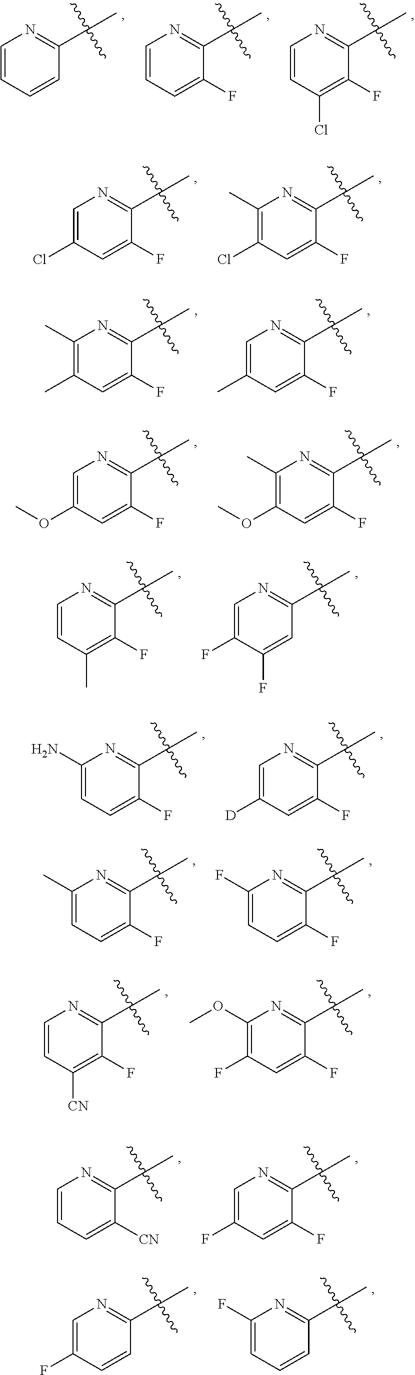

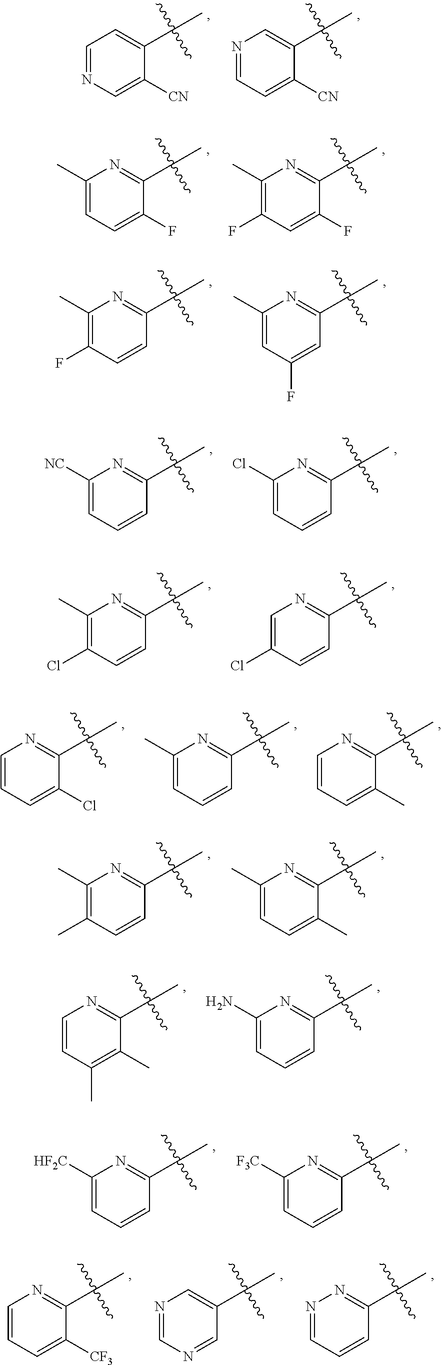

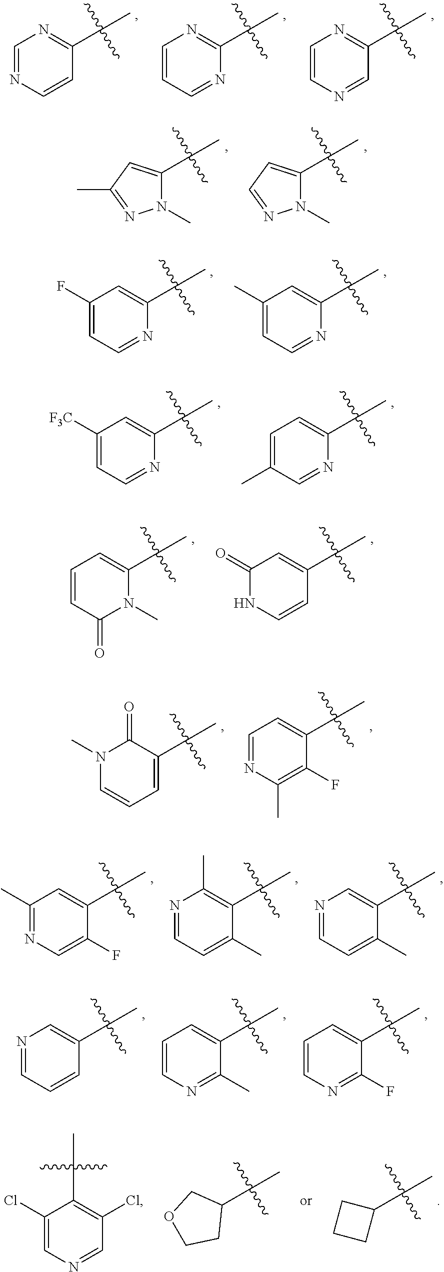

In one embodiment, R³ and R⁴ are, each independently, H or C₁-C₄ alkyl. In some embodiments, R³ and R⁴ are, each independently, H or C₁ alkyl. In some embodiments, R³ and R⁴ each are H. In some embodiments, R³ and R⁴ each are C₁ alkyl. In some embodiments, one of R³ or R⁴ is H and the other one of R³ or R⁴ is C₁ alkyl. In some embodiments, C₁ alkyl of R³ and R⁴ is methyl.

In one embodiment, B is C₃-C₈ cycloalkyl, C₃-C₈ heterocycloalkyl, a fused bicyclic, a bridged bicyclic, a spirocyclic, or absent. In some embodiments, the C₃-C₈ cycloalkyl, C₃-C₈ heterocycloalkyl, a fused bicyclic, a bridged bicyclic, or a spirocyclic of B is further substituted with fluoro, —OH, or methyl. In some certain embodiments, the C₃-C₈ heterocycloalkyl of B is sulfoximine or sulfone. In some certain embodiments, the C₃-C₈ heterocycloalkyl of B is sulfoximine. In some certain embodiments, the C₃-C₈ heterocycloalkyl of B is sulfone. In some certain embodiments, the C₃-C₈ heterocycloalkyl of B is

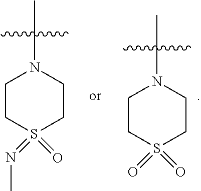

In some certain embodiments, the C₃-C₈ heterocycloalkyl of B is

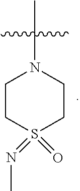 [SMILES: *N1CCS(=O)(=NC)CC1]

In some certain embodiments, the C₃-C₈ heterocycloalkyl of B is

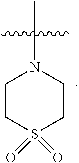 [SMILES: *N1CCS(=O)(=O)CC1]

In some embodiments, B is C₆-C₇ cycloalkyl, C₄-C₆ heterocycloalkyl, a fused bicyclic, a bridged bicyclic, or a spirocyclic. In some specific embodiments, B is C₆ cycloalkyl, C₄-C₅ heterocycloalkyl, a fused bicyclic, a bridged bicyclic, or a spirocyclic. In some embodiments, the fused bicyclic of B is octahydrocyclopenta[c]pyrrole, 3-azabicyclo[3.2.0]heptane, or 3-azabicyclo[3.1.0]hexane. In some embodiments, the bridged bicyclic of B is 3-azabicyclo[3.2.1]octane, 8-azabicyclo[3.2.1]octane, or 2-azabicyclo[2.2.1]heptane. In some embodiments, the spirocyclic of B is 1-azaspiro[4.5]decane, 2-azaspiro[4.5]decane, 3-azaspiro[5.5]undecane, 2-azaspiro[3.5]nonane, 2-azaspiro[3.3]heptane, 7-azaspiro[3.5]nonane, 1-azaspiro[3.3]heptane, or spiro[3.3]heptane. In some embodiments, B has one of the following structures:

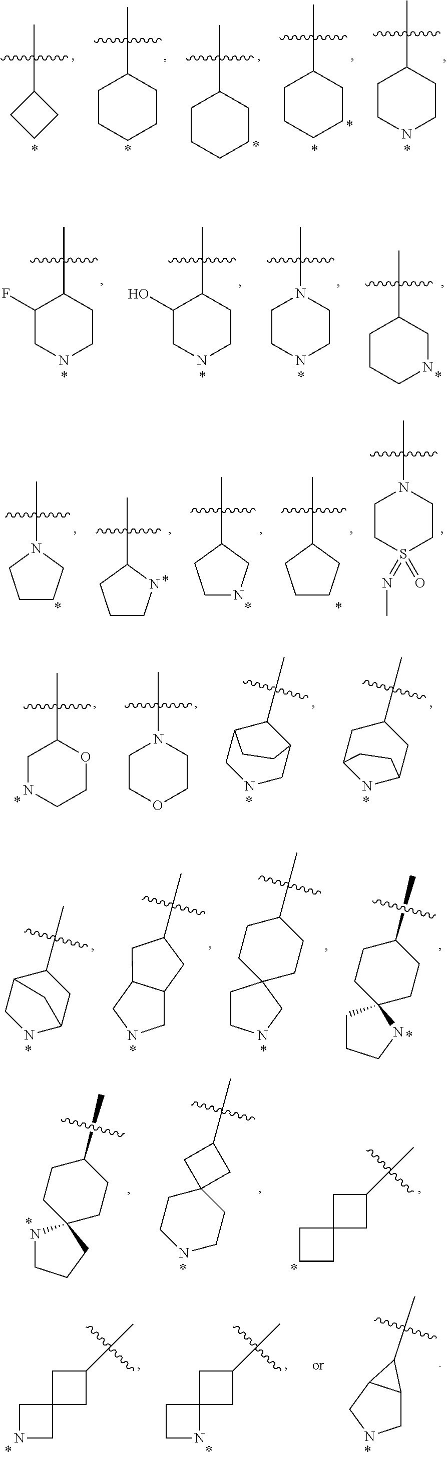

wherein * indicates a location of a bond to R⁵. In some embodiments, B is

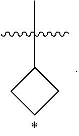 [SMILES: *C1CCC1]

In some embodiments, B is

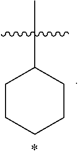 [SMILES: *C1CCCCC1]

In some embodiments, B is

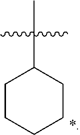 [SMILES: *C1CCCCC1]

In some embodiments, B is

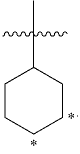 [SMILES: *C1CCCCC1]

In some embodiments, B is

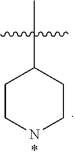 [SMILES: *C1CCNCC1]

In some embodiments, B is

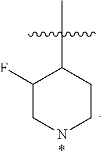 [SMILES: *C1CCNCC1F]

In some embodiments, B is

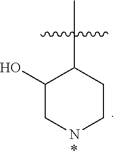 [SMILES: *C1CCNCC1O]

In some embodiments, B is

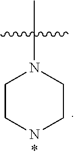 [SMILES: *N1CCNCC1]

In some embodiments, B is

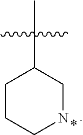 [SMILES: *C1CCCNC1]

In some embodiments, B is

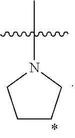 [SMILES: *N1CCCC1]

In some embodiments, B is

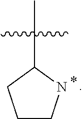 [SMILES: *C1CCCN1]

In some embodiments, B is

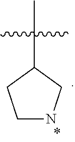 [SMILES: *C1CCNC1]

In some embodiments, B is

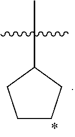 [SMILES: *C1CCCC1]

In some embodiments, B is

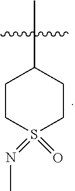 [SMILES: *C1CCS(=O)(=NC)CC1]

In some embodiments, B is

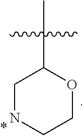 [SMILES: *C1CNCCO1]

In some embodiments, B is

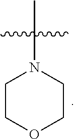 [SMILES: *N1CCOCC1]

In some embodiments, B is

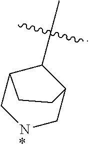 [SMILES: *C1C2CCC1CNC2]

In some embodiments, B is

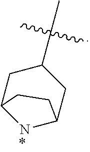 [SMILES: *C1CC2CCC(C1)N2]

In some embodiments, B is

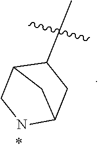 [SMILES: *C1CC2CC1CN2]

In some embodiments, B is

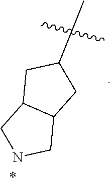 [SMILES: *C1CC2CNCC2C1]

In some embodiments, B is

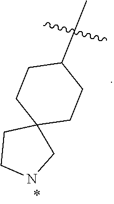 [SMILES: *C1CCC2(CCNC2)CC1]

In some embodiments, B is

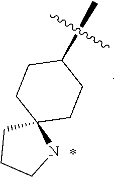

In some embodiments, B is

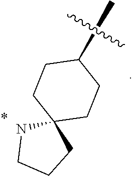

In some embodiments, B is

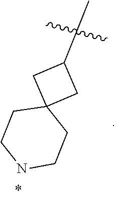 [SMILES: *C1CC2(CCNCC2)C1]

In some embodiments, B is

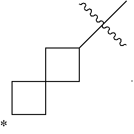 [SMILES: *C1CC2(CCC2)C1]

In some embodiments, B is

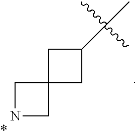 [SMILES: *C1CC2(CNC2)C1]

In some embodiments, B is

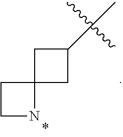 [SMILES: *C1CC2(CCN2)C1]

In some embodiments, B is

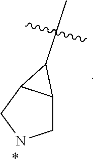 [SMILES: *C1C2CNCC12]

In some embodiments, B is substituted with one or more R⁵. In some embodiments, B is substituted with one, two, or three R⁵. In some embodiments, B is absent. When B is absent, Y is directly attached to R⁵. When B is absent and Y is a direct bond, A is attached to R⁵.

In some embodiments, R⁵ is —H, —OH, —CN, —F, C₁-C₄ alkyl, C₃-C₆ cycloalkyl, C₁-C₄ heteroalkyl, 6 membered heteroaryl, C₄-C₆ heterocycloalkyl —OC(═O)R⁶, —CH₂C(═O)OR⁶, —NR⁷R⁸, —NR⁷C(═O)R⁶, —NR⁷C(═O)OR⁶, —CH₂C(═O)NR⁷N⁸, —NR⁷R⁸CH₂C(═O)NR⁷N⁸, —CH₂NR⁷C(═O)R⁶, sulfonylmethane, oxo, phosphate, or a combination thereof.

In some embodiments, R⁵ is —H. In some embodiments, R⁵ is —OH. In some embodiments, R⁵ is —CN. In some embodiments, R⁵ is —F. In some embodiments, R⁵ is C₁-C₄ alkyl. In some embodiments, R⁵ is C₃-C₆ cycloalkyl. In some embodiments, R⁵ is C₁-C₄ heteroalkyl. In some embodiments, R⁵ is 6 membered heteroaryl. In some embodiments, R⁵ is C₄-C₆ heterocycloalkyl —OC(═O)R⁶. In some embodiments, R⁵ is —CH₂C(═O)OR⁶. In some embodiments, R⁵ is —NR⁷R⁸. In some embodiments, R⁵ is —NR⁷C(═O)R⁶. In some embodiments, R⁵ is —NR⁷C(═O)OR⁶. In some embodiments, R⁵ is —CH₂C(═O)NR⁷N⁸. In some embodiments, R⁵ is —NR⁷R⁸CH₂C(═O)NR⁷N⁸. In some embodiments, R⁵ is —CH₂NR⁷C(═O)R⁶. In some embodiments, R⁵ is sulfonylmethane. In some embodiments, R⁵ is oxo. In some embodiments, R⁵ is phosphate.

In some embodiments, the C₁-C₆ heteroalkyl of R⁵ is C₁-C₆ deutro-alkyl. In some embodiments, the C₁-C₆ heteroalkyl of R⁵ is C₁ deutro-alkyl. In some embodiments, the C₁-C₆ heteroalkyl of R⁵ is C₂ deutro-alkyl. In some embodiments, the C₁-C₆ heteroalkyl of R⁵ is C₃ deutro-alkyl. In some embodiments, the C₁-C₆ heteroalkyl of R⁵ is C₄ deutro-alkyl. In some embodiments, the C₁-C₆ heteroalkyl of R⁵ is C₅ deutro-alkyl. In some embodiments, the C₁-C₆ heteroalkyl of R⁵ is C₆ deutro-alkyl.

In one embodiment, R⁶ is a hydrogen, C₁-C₄ alkyl, C₁-C₅ heteroalkyl, or C₄-C₆ heterocycloalkyl, wherein the C₄-C₆ heterocycloalkyl is substituted with

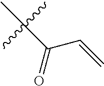 [SMILES: *C(=O)C=C]

or halo. In some embodiments, R⁶ is a hydrogen. In some embodiments, R⁶ is C₁-C₄ alkyl. In some embodiments, R⁶ is C₁-C₅ heteroalkyl. In some embodiments, R⁶ is C₄-C₆ heterocycloalkyl. In some certain embodiments, the C₄-C₆ heterocycloalkyl is substituted with

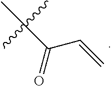 [SMILES: *C(=O)C=C]

In some certain embodiments, the C₄-C₆ heterocycloalkyl is substituted with halo. In some embodiments, R⁶ is C₁-C₃ alkyl, C₁-C₄ heteroalkyl, or C₄-C₅ heterocycloalkyl. In some embodiments, R⁶ is C₁-C₃ alkyl. In some embodiments, R⁶ is C₁-C₄ heteroalkyl. In some embodiments, R⁶ is C₄-C₅ heterocycloalkyl. In some embodiments, the C₁-C₄ heteroalkyl of R⁶ is C₁-C₄ fluoroalkyl. In some embodiments, the C₁-C₄ heteroalkyl of R⁶ is C₁ fluoroalkyl. In some embodiments, the C₁-C₄ heteroalkyl of R⁶ is C₂ fluoroalkyl. In some embodiments, the C₁-C₄ heteroalkyl of R⁶ is Ca fluoroalkyl. In some embodiments, the C₁-C₄ heteroalkyl of R⁶ is C₄ fluoroalkyl.

In some certain embodiments, the C₄-C₆ heterocycloalkyl of R⁶ is pyrrolidine or 1-methyl pyrrolidine. In some certain embodiments, the C₄-C₆ heterocycloalkyl of R⁶ is pyrrolidine. In some certain embodiments, the C₄-C₆ heterocycloalkyl of R⁶ is 1-methyl pyrrolidine.

In some embodiments, the C₄-C₆ heterocycloalkyl of R⁶ is substituted with halo. In some embodiments, the C₄-C₆ heterocycloalkyl of R⁶ is substituted with fluoro. In some embodiments, the C₄-C₆ heterocycloalkyl of R⁶ is substituted with

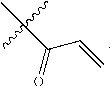 [SMILES: *C(=O)C=C]

In some embodiments, the C₄-C₆ heterocycloalkyl of R⁶ substituted with

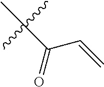 [SMILES: *C(=O)C=C]

is

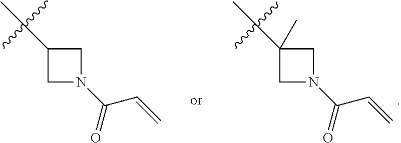

In some embodiments, the C₄-C₆ heterocycloalkyl of R⁶ substituted with

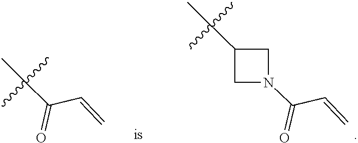

In some embodiments, the C₄-C₆ heterocycloalkyl of R⁶ substituted with

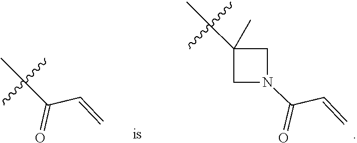

In some embodiments, the C₄-C₆ heterocycloalkyl of R⁶ substituted with fluoro is

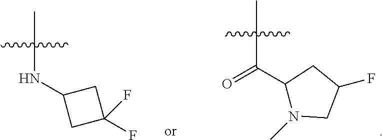

In some embodiments, the C₄-C₆ heterocycloalkyl of R⁶ substituted with fluoro is

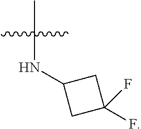 [SMILES: *NC1CC(F)(F)C1]

In some embodiments, the C₄-C₆ heterocycloalkyl of R⁶ substituted with fluoro is

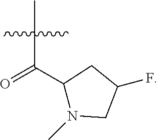 [SMILES: *C(=O)C1CC(F)CN1C]

In some embodiments, R⁷ and R⁸ are, each independently, H, C₁-C₃ alkyl, C₁-C₃ alkyl alcohol, sulfonylmethane, C₃-C₄ cycloalkyl, or 6 membered heteroaryl, wherein the C₁-C₃ alkyl or C₃-C₄ cycloalkyl is optionally substituted with fluoro or C₃-C₄ cycloalkyl.

In some embodiments, the 5-6 membered heteroaryl of R⁵ is

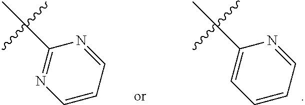 [SMILES: *c1ncccn1 ; *c1ccccn1]

the 5-6 membered heteroaryl of R⁵ is

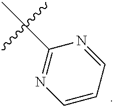 [SMILES: *c1ncccn1]

the 5-6 membered heteroaryl of R⁵ is

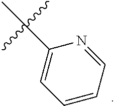 [SMILES: *c1ccccn1]

In some embodiments, the C₃-C₈ heterocycloalkyl of R⁵ is

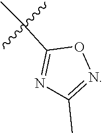 [SMILES: *c1nc(C)no1]

In some embodiments, the C₃-C₈ cycloalkyl of R⁵ is

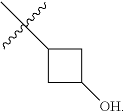 [SMILES: *C1CC(O)C1]

In some embodiments, the oxo of R⁵ is

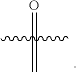 [SMILES: *=O]

In some embodiments, the phosphate of R⁵ is

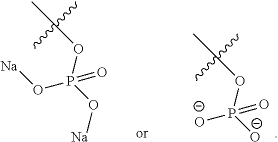

In some embodiments, the phosphate of R⁵ is

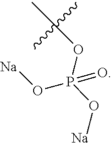

In some embodiments, the phosphate of R⁵ is

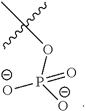 [SMILES: *OP(=O)([O-])[O-]]

In some embodiments, R⁵ has one of the following structures:

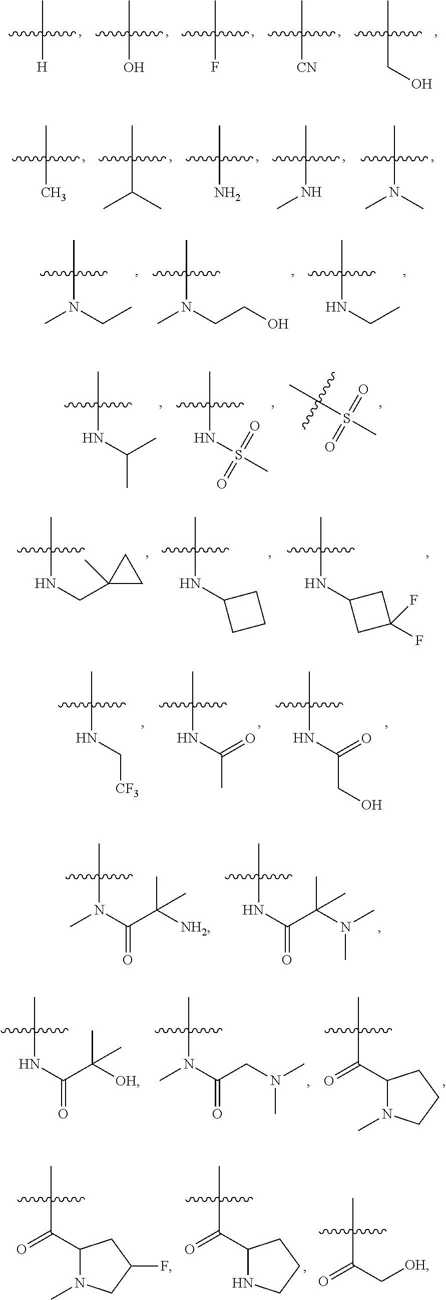

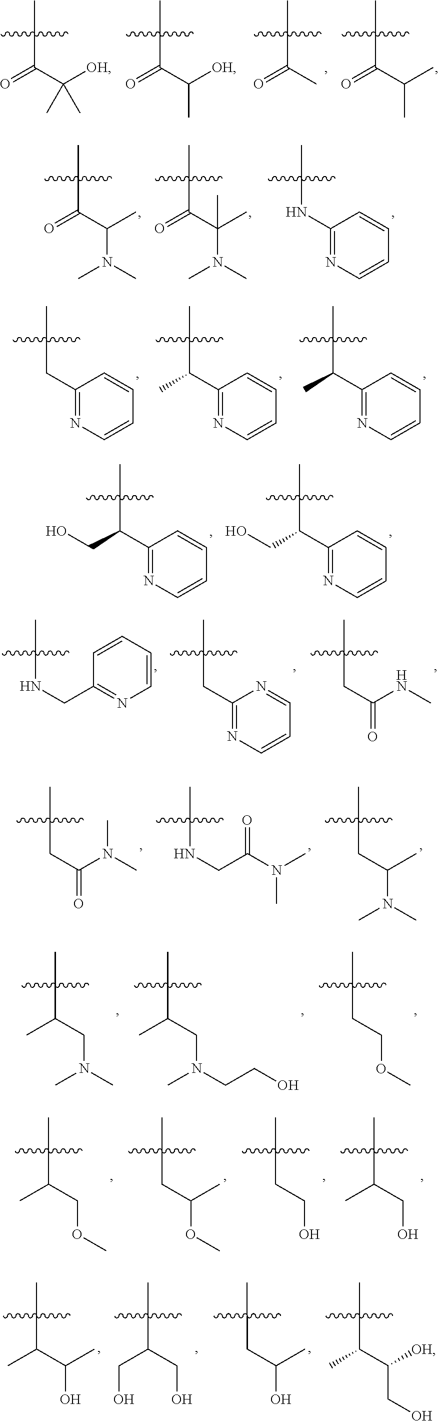

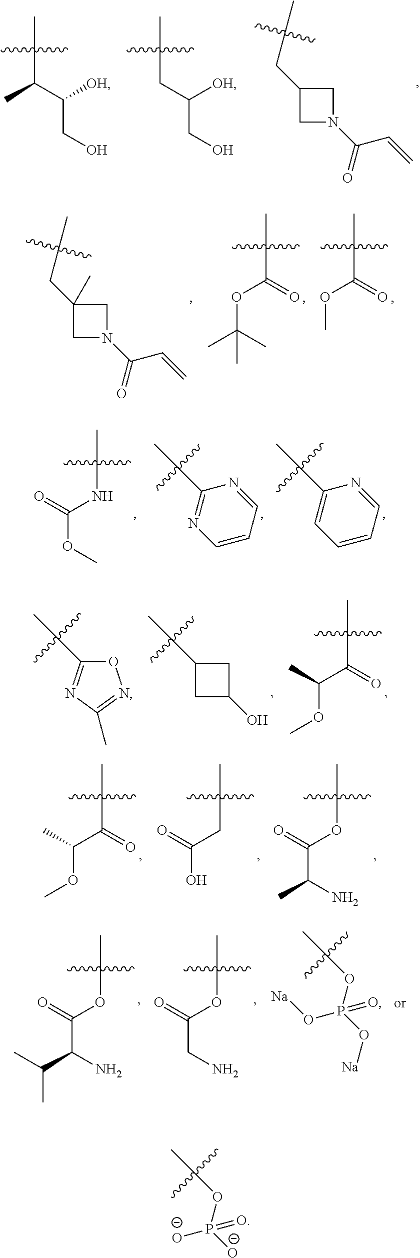

In some embodiments, R⁵ is

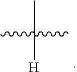 [SMILES: *[H]]

In some embodiments, R⁵ is

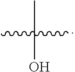 [SMILES: *O]

In some embodiments, R⁵ is

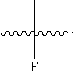 [SMILES: *F]

In some embodiments, R⁵ is

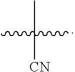 [SMILES: *C#N]

In some embodiments, R⁵ is

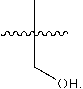 [SMILES: *CO]

In some embodiments, R⁵ is

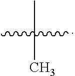

In some embodiments, R⁵ is

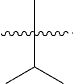 [SMILES: *C(C)C]

In some embodiments, R⁵ is

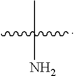 [SMILES: *N]

In some embodiments, R⁵ is

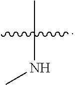 [SMILES: *NC]

In some embodiments, R⁵ is

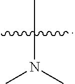

In some embodiments, R⁵ is

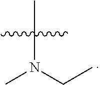 [SMILES: *N(C)CC]

In some embodiments, R⁵ is

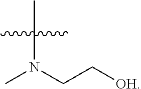 [SMILES: *N(C)CCO]

In some embodiments, R⁵ is

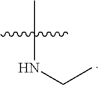 [SMILES: *NCC]

In some embodiments, R⁵ is

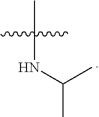 [SMILES: *NC(C)C]

In some embodiments, R⁵ is

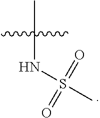 [SMILES: *NS(C)(=O)=O]

In some embodiments, R⁵ is

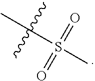 [SMILES: *S(C)(=O)=O]

In some embodiments, R⁵ is

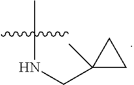 [SMILES: *NCC1(C)CC1]

In some embodiments, R⁵ is

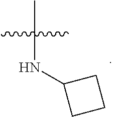 [SMILES: *NC1CCC1]

In some embodiments, R⁵ is

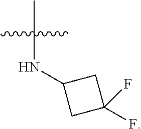 [SMILES: *NC1CC(F)(F)C1]

In some embodiments, R⁵ is

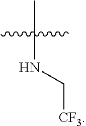 [SMILES: *NCC(F)(F)F]

In some embodiments, R⁵ is

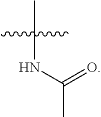 [SMILES: *NC(C)=O]

In some embodiments, R⁵ is

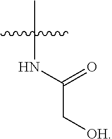 [SMILES: *NC(=O)CO]

In some embodiments, R⁵ is

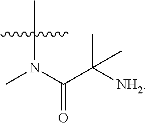 [SMILES: *N(C)C(=O)C(C)(C)N]

In some embodiments, R⁵ is

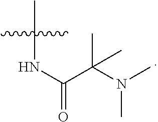 [SMILES: *NC(=O)C(C)(C)N(C)C]

In some embodiments, R⁵ is

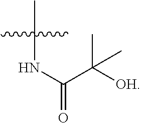 [SMILES: *NC(=O)C(C)(C)O]

In some embodiments, R⁵ is

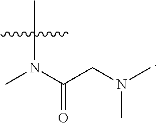

In some embodiments, R⁵ is

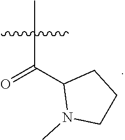 [SMILES: *C(=O)C1CCCN1C]

In some embodiments, R⁵ is

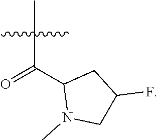 [SMILES: *C(=O)C1CC(F)CN1C]

In some embodiments, R⁵ is

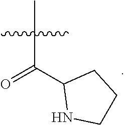 [SMILES: *C(=O)C1CCCN1]

In some embodiments, R⁵ is

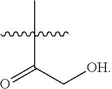 [SMILES: *C(=O)CO]

In some embodiments, R⁵ is

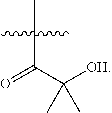 [SMILES: *C(=O)C(C)(C)O]

In some embodiments, R⁵ is

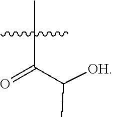 [SMILES: *C(=O)C(C)O]

In some embodiments, R⁵ is

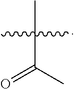 [SMILES: *C(C)=O]

In some embodiments, R⁵ is

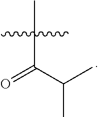 [SMILES: *C(=O)C(C)C]

In some embodiments, R⁵ is

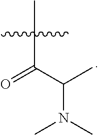 [SMILES: *C(=O)C(C)N(C)C]

In some embodiments, R⁵ is

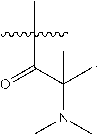 [SMILES: *C(=O)C(C)(C)N(C)C]

In some embodiments, R⁵ is

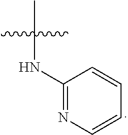 [SMILES: *Nc1ccccn1]

In some embodiments, R⁵ is

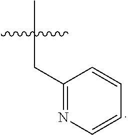 [SMILES: *Cc1ccccn1]

In some embodiments, R⁵ is

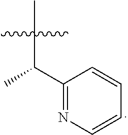 [SMILES: *[C@@H](C)c1ccccn1]

In some embodiments, R⁵ is

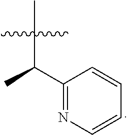 [SMILES: *[C@H](C)c1ccccn1]

In some embodiments, R⁵ is

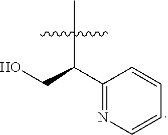 [SMILES: *[C@H](CO)c1ccccn1]

In some embodiments, R⁵ is

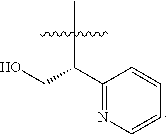 [SMILES: *[C@@H](CO)c1ccccn1]

In some embodiments, R⁵ is

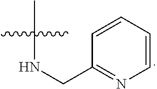 [SMILES: *NCc1ccccn1]

In some embodiments, R⁵ is

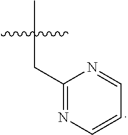 [SMILES: *Cc1ncccn1]

In some embodiments, R⁵ is

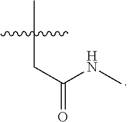 [SMILES: *CC(=O)NC]

In some embodiments, R⁵ is

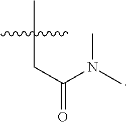 [SMILES: *CC(=O)N(C)C]

In some embodiments, R⁵ is

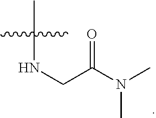 [SMILES: *NCC(=O)N(C)C]

In some embodiments, R⁵ is

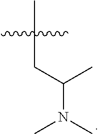 [SMILES: *CC(C)N(C)C]

In some embodiments, R⁵ is

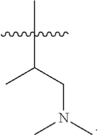 [SMILES: *C(C)CN(C)C]

In some embodiments, R⁵ is

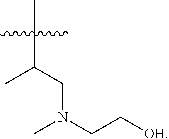 [SMILES: *C(C)CN(C)CCO]

In some embodiments, R⁵ is

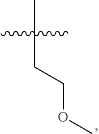 [SMILES: *CCOC]

In some embodiments, R⁵ is

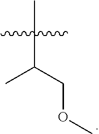 [SMILES: *C(C)COC]

In some embodiments, R⁵ is

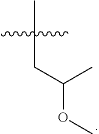 [SMILES: *CC(C)OC]

In some embodiments, R⁵ is

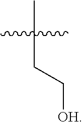 [SMILES: *CCO]

In some embodiments, R⁵ is

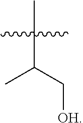 [SMILES: *C(C)CO]

In some embodiments, R⁵ is

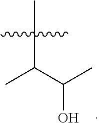 [SMILES: *C(C)C(C)O]

In some embodiments, R⁵ is

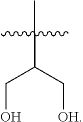 [SMILES: *C(CO)CO]

In some embodiments, R⁵ is

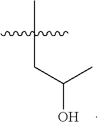 [SMILES: *CC(C)O]

In some embodiments, R⁵ is

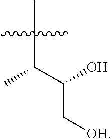 [SMILES: *[C@@H](C)[C@H](O)CO]

In some embodiments, R⁵ is

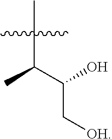 [SMILES: *[C@H](C)[C@H](O)CO]

In some embodiments, R⁵ is

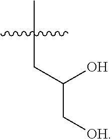 [SMILES: *CC(O)CO]

In some embodiments, R⁵ is

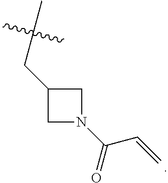 [SMILES: *CC1CN(C(=O)C=C)C1]

In some embodiments, R⁵ is

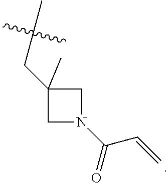 [SMILES: *CC1(C)CN(C(=O)C=C)C1]

In some embodiments, R⁵ is

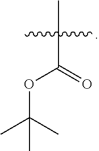 [SMILES: *C(=O)OC(C)(C)C]

In some embodiments, R⁵ is

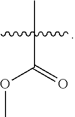 [SMILES: *C(=O)OC]

In some embodiments, R⁵ is

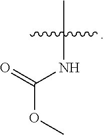 [SMILES: *NC(=O)OC]

In some embodiments, R⁵ is

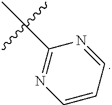 [SMILES: *c1ncccn1]

In some embodiments, R⁵ is

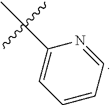 [SMILES: *c1ccccn1]

In some embodiments, R⁵ is

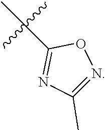 [SMILES: *c1nc(C)no1]

In some embodiments, R⁵ is

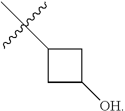 [SMILES: *C1CC(O)C1]

In some embodiments, R⁵ is

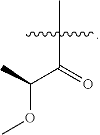 [SMILES: *C(=O)[C@H](C)OC]

In some embodiments, R⁵ is

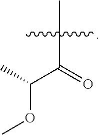 [SMILES: *C(=O)[C@@H](C)OC]

In some embodiments, R⁵ is

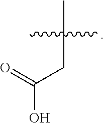 [SMILES: *CC(=O)O]

In some embodiments, R⁵ is

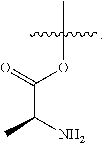 [SMILES: *OC(=O)[C@H](C)N]

In some embodiments, R⁵ is

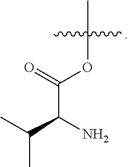 [SMILES: *OC(=O)[C@@H](N)C(C)C]

In some embodiments, R₅ is

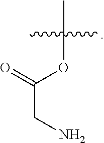 [SMILES: *OC(=O)CN]

In some embodiments, R⁵ is

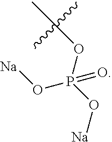

In some embodiments, R⁵ is

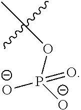 [SMILES: *OP(=O)([O-])[O-]]

In one embodiment, the compound of formula (I)-(IL) has one of the following structures shown in Table B below.

TABLE B
List of Compounds of (I)-(IL)

| Compound ID | Structure |
|---|---|
| 1 | 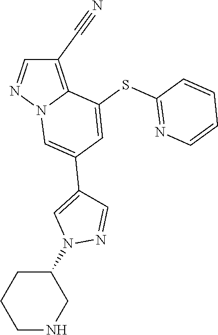 [SMILES: N#Cc1cnn2cc(-c3cnn([C@H]4CCCNC4)c3)cc(Sc3ccccn3)c12] |
| 2 | 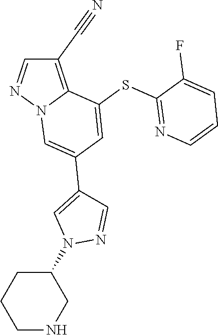 [SMILES: N#Cc1cnn2cc(-c3cnn([C@H]4CCCNC4)c3)cc(Sc3ncccc3F)c12] |
| 3 | 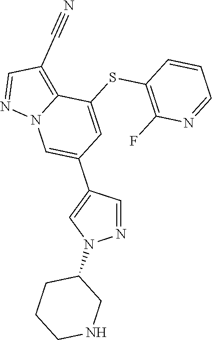 [SMILES: N#Cc1cnn2cc(-c3cnn([C@H]4CCCNC4)c3)cc(Sc3cccnc3F)c12] |
| 4 | 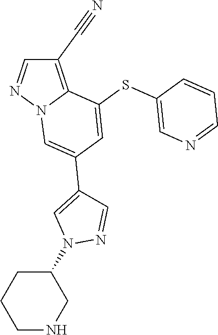 [SMILES: N#Cc1cnn2cc(-c3cnn([C@H]4CCCNC4)c3)cc(Sc3cccnc3)c12] |
| 5 | 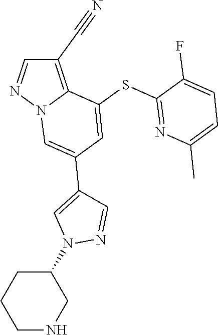 [SMILES: Cc1ccc(F)c(Sc2cc(-c3cnn([C@H]4CCCNC4)c3)cn3ncc(C#N)c23)n1] |
| 6 | 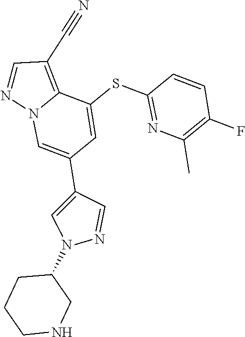 [SMILES: Cc1nc(Sc2cc(-c3cnn([C@H]4CCCNC4)c3)cn3ncc(C#N)c23)ccc1F] |
| 7 | 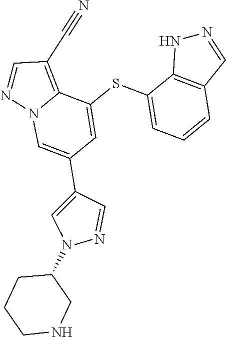 [SMILES: N#Cc1cnn2cc(-c3cnn([C@H]4CCCNC4)c3)cc(Sc3cccc4cn[nH]c34)c12] |
| 8 | 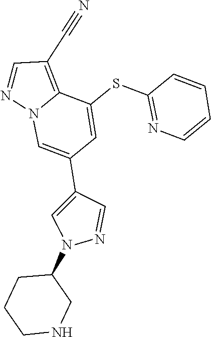 [SMILES: N#Cc1cnn2cc(-c3cnn([C@@H]4CCCNC4)c3)cc(Sc3ccccn3)c12] |
| 9 | 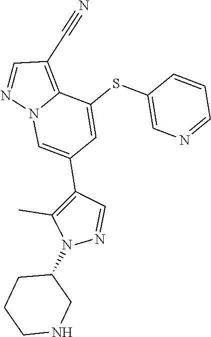 [SMILES: Cc1c(-c2cc(Sc3cccnc3)c3c(C#N)cnn3c2)cnn1[C@H]1CCCNC1] |
| 10 | 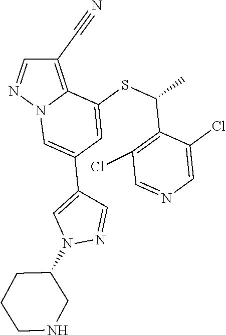 [SMILES: C[C@@H](Sc1cc(-c2cnn([C@H]3CCCNC3)c2)cn2ncc(C#N)c12)c1c(Cl)cncc1Cl] |
| 11 | 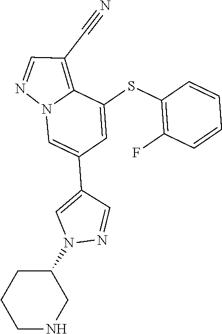 [SMILES: N#Cc1cnn2cc(-c3cnn([C@H]4CCCNC4)c3)cc(Sc3ccccc3F)c12] |
| 12 | 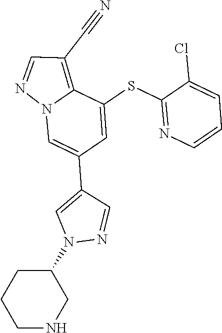 [SMILES: N#Cc1cnn2cc(-c3cnn([C@H]4CCCNC4)c3)cc(Sc3ncccc3Cl)c12] |
| 13 | 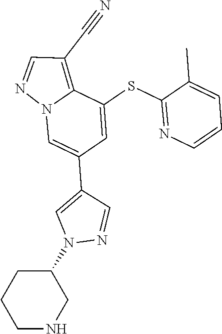 [SMILES: Cc1cccnc1Sc1cc(-c2cnn([C@H]3CCCNC3)c2)cn2ncc(C#N)c12] |
| 14 | 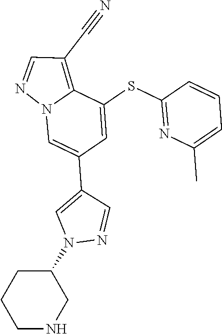 [SMILES: Cc1cccc(Sc2cc(-c3cnn([C@H]4CCCNC4)c3)cn3ncc(C#N)c23)n1] |
| 15 | 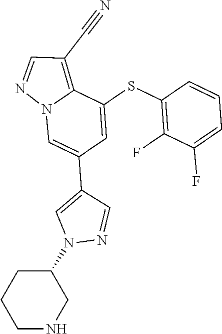 [SMILES: N#Cc1cnn2cc(-c3cnn([C@H]4CCCNC4)c3)cc(Sc3cccc(F)c3F)c12] |
| 16 | 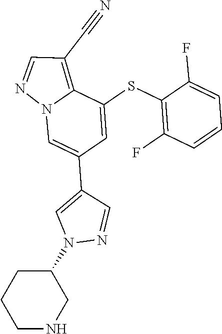 [SMILES: N#Cc1cnn2cc(-c3cnn([C@H]4CCCNC4)c3)cc(Sc3c(F)cccc3F)c12] |
| 17 | 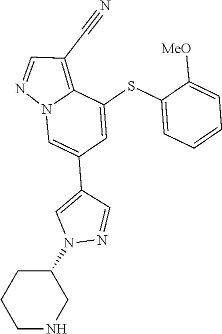 [SMILES: COc1ccccc1Sc1cc(-c2cnn([C@H]3CCCNC3)c2)cn2ncc(C#N)c12] |
| 18 | 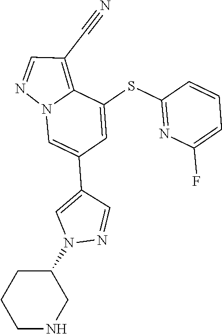 [SMILES: N#Cc1cnn2cc(-c3cnn([C@H]4CCCNC4)c3)cc(Sc3cccc(F)n3)c12] |
| 19 | 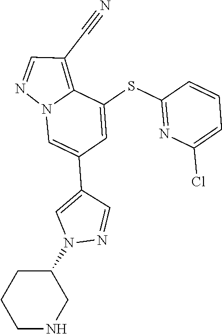 [SMILES: N#Cc1cnn2cc(-c3cnn([C@H]4CCCNC4)c3)cc(Sc3cccc(Cl)n3)c12] |
| 20 | 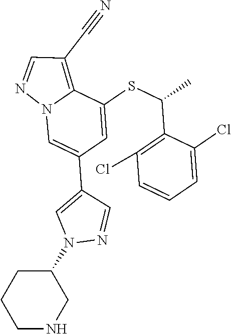 [SMILES: C[C@@H](Sc1cc(-c2cnn([C@H]3CCCNC3)c2)cn2ncc(C#N)c12)c1c(Cl)cccc1Cl] |
| 21 | 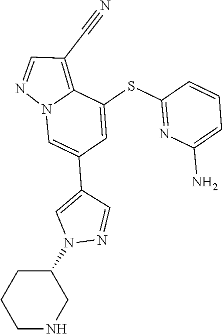 [SMILES: N#Cc1cnn2cc(-c3cnn([C@H]4CCCNC4)c3)cc(Sc3cccc(N)n3)c12] |
| 22 | 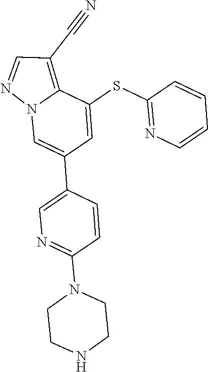 [SMILES: N#Cc1cnn2cc(-c3ccc(N4CCNCC4)nc3)cc(Sc3ccccn3)c12] |
| 23 | 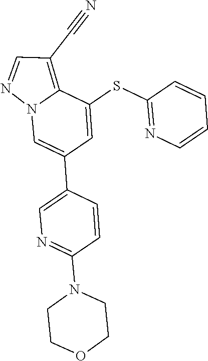 [SMILES: N#Cc1cnn2cc(-c3ccc(N4CCOCC4)nc3)cc(Sc3ccccn3)c12] |
| 24 | 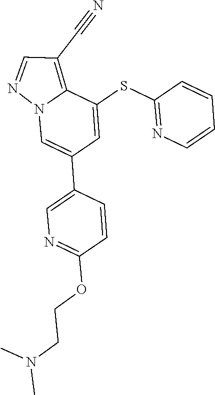 [SMILES: CN(C)CCOc1ccc(-c2cc(Sc3ccccn3)c3c(C#N)cnn3c2)cn1] |
| 25 | 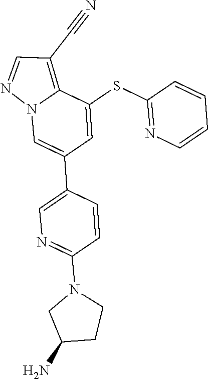 [SMILES: N#Cc1cnn2cc(-c3ccc(N4CC[C@@H](N)C4)nc3)cc(Sc3ccccn3)c12] |
| 26 | 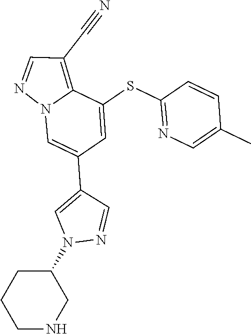 [SMILES: Cc1ccc(Sc2cc(-c3cnn([C@H]4CCCNC4)c3)cn3ncc(C#N)c23)nc1] |
| 27 | 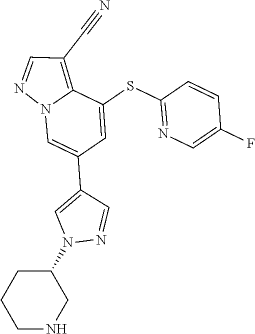 [SMILES: N#Cc1cnn2cc(-c3cnn([C@H]4CCCNC4)c3)cc(Sc3ccc(F)cn3)c12] |
| 28 | 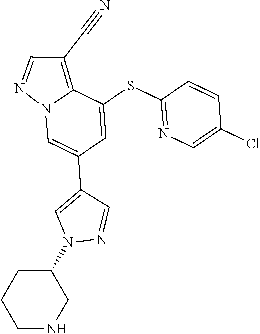 [SMILES: N#Cc1cnn2cc(-c3cnn([C@H]4CCCNC4)c3)cc(Sc3ccc(Cl)cn3)c12] |
| 29 | 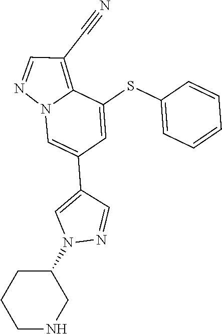 [SMILES: N#Cc1cnn2cc(-c3cnn([C@H]4CCCNC4)c3)cc(Sc3ccccc3)c12] |
| 30 | 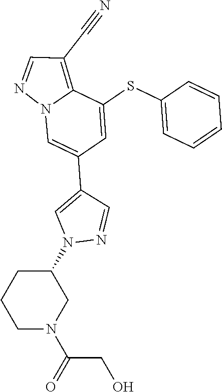 [SMILES: N#Cc1cnn2cc(-c3cnn([C@H]4CCCN(C(=O)CO)C4)c3)cc(Sc3ccccc3)c12] |
| 31 | 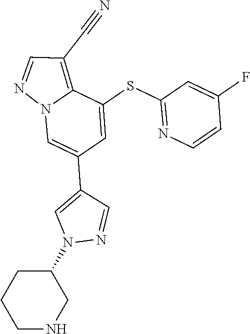 [SMILES: N#Cc1cnn2cc(-c3cnn([C@H]4CCCNC4)c3)cc(Sc3cc(F)ccn3)c12] |
| 32 | 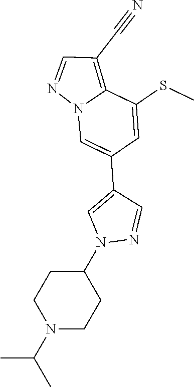 [SMILES: CSc1cc(-c2cnn(C3CCN(C(C)C)CC3)c2)cn2ncc(C#N)c12] |
| 33 | 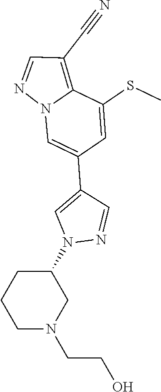 [SMILES: CSc1cc(-c2cnn([C@H]3CCCN(CCO)C3)c2)cn2ncc(C#N)c12] |
| 34 | 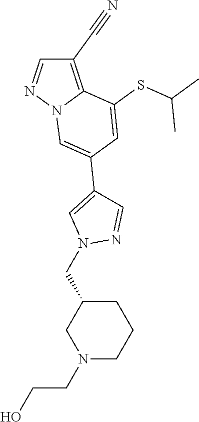 [SMILES: CC(C)Sc1cc(-c2cnn(C[C@@H]3CCCN(CCO)C3)c2)cn2ncc(C#N)c12] |
| 35 | 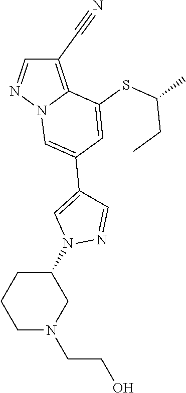 [SMILES: CC[C@@H](C)Sc1cc(-c2cnn([C@H]3CCCN(CCO)C3)c2)cn2ncc(C#N)c12] |
| 36 | 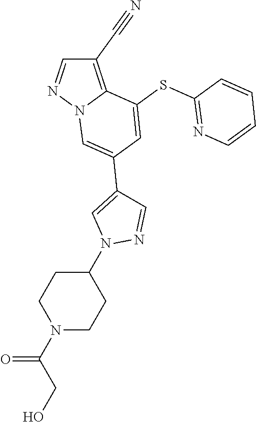 [SMILES: N#Cc1cnn2cc(-c3cnn(C4CCN(C(=O)CO)CC4)c3)cc(Sc3ccccn3)c12] |
| 37 | 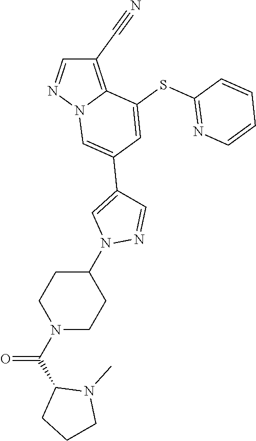 [SMILES: CN1CCC[C@@H]1C(=O)N1CCC(n2cc(-c3cc(Sc4ccccn4)c4c(C#N)cnn4c3)cn2)CC1] |
| 38 | 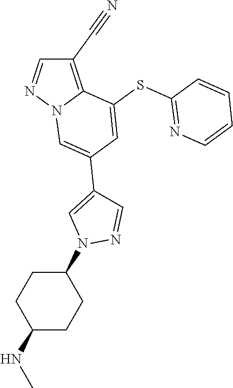 [SMILES: CN[C@H]1CC[C@@H](n2cc(-c3cc(Sc4ccccn4)c4c(C#N)cnn4c3)cn2)CC1] |
| 39 | 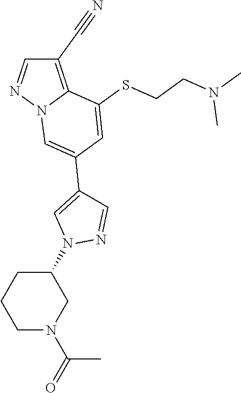 [SMILES: CC(=O)N1CCC[C@H](n2cc(-c3cc(SCCN(C)C)c4c(C#N)cnn4c3)cn2)C1] |
| 40 | 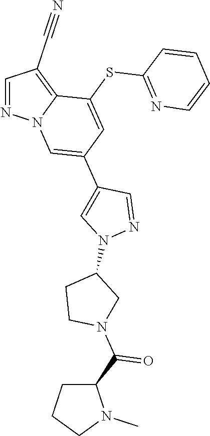 [SMILES: CN1CCC[C@H]1C(=O)N1CC[C@H](n2cc(-c3cc(Sc4ccccn4)c4c(C#N)cnn4c3)cn2)C1] |
| 41 | 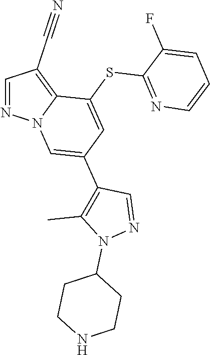 [SMILES: Cc1c(-c2cc(Sc3ncccc3F)c3c(C#N)cnn3c2)cnn1C1CCNCC1] |
| 42 | 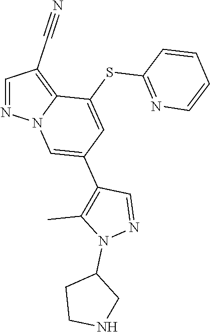 [SMILES: Cc1c(-c2cc(Sc3ccccn3)c3c(C#N)cnn3c2)cnn1C1CCNC1] |
| 43 | 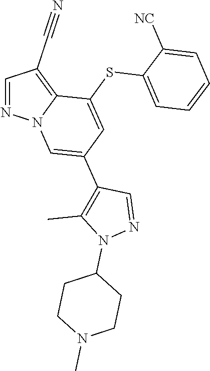 [SMILES: Cc1c(-c2cc(Sc3ccccc3C#N)c3c(C#N)cnn3c2)cnn1C1CCN(C)CC1] |
| 44 | 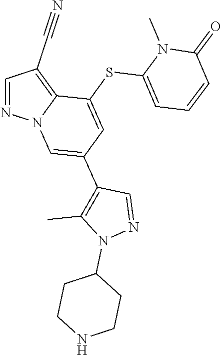 [SMILES: Cc1c(-c2cc(Sc3cccc(=O)n3C)c3c(C#N)cnn3c2)cnn1C1CCNCC1] |
| 45 | 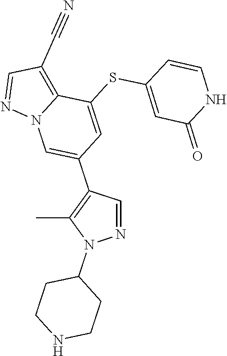 [SMILES: Cc1c(-c2cc(Sc3cc[nH]c(=O)c3)c3c(C#N)cnn3c2)cnn1C1CCNCC1] |
| 46 | 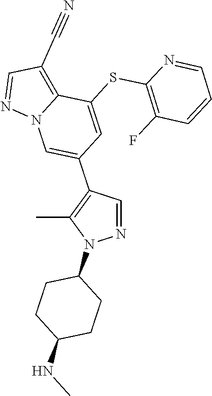 [SMILES: CN[C@H]1CC[C@@H](n2ncc(-c3cc(Sc4ncccc4F)c4c(C#N)cnn4c3)c2C)CC1] |
| 47 | 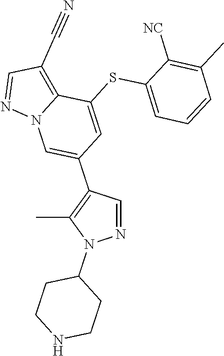 [SMILES: Cc1cccc(Sc2cc(-c3cnn(C4CCNCC4)c3C)cn3ncc(C#N)c23)c1C#N] |
| 48 | 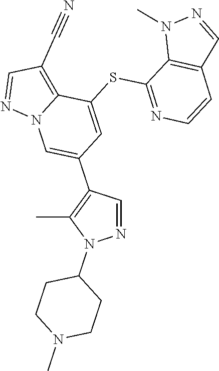 [SMILES: Cc1c(-c2cc(Sc3nccc4cnn(C)c34)c3c(C#N)cnn3c2)cnn1C1CCN(C)CC1] |
| 49 | 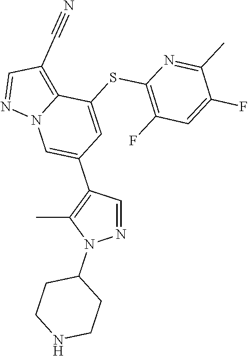 [SMILES: Cc1nc(Sc2cc(-c3cnn(C4CCNCC4)c3C)cn3ncc(C#N)c23)c(F)cc1F] |
| 50 | 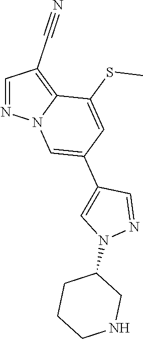 [SMILES: CSc1cc(-c2cnn([C@H]3CCCNC3)c2)cn2ncc(C#N)c12] |
| 51 | 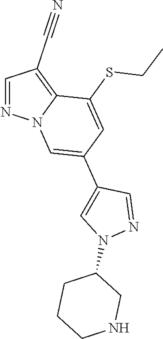 [SMILES: CCSc1cc(-c2cnn([C@H]3CCCNC3)c2)cn2ncc(C#N)c12] |
| 52 | 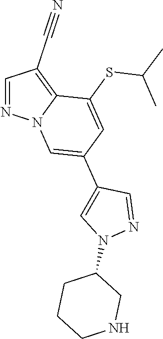 [SMILES: CC(C)Sc1cc(-c2cnn([C@H]3CCCNC3)c2)cn2ncc(C#N)c12] |
| 53 | 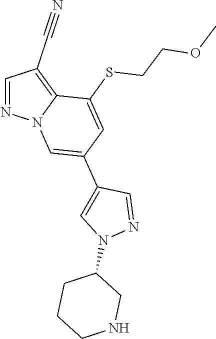 [SMILES: COCCSc1cc(-c2cnn([C@H]3CCCNC3)c2)cn2ncc(C#N)c12] |
| 54 | 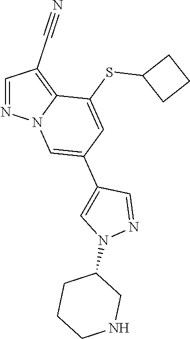 [SMILES: N#Cc1cnn2cc(-c3cnn([C@H]4CCCNC4)c3)cc(SC3CCC3)c12] |
| 55 | 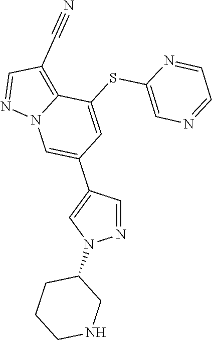 [SMILES: N#Cc1cnn2cc(-c3cnn([C@H]4CCCNC4)c3)cc(Sc3cnccn3)c12] |
| 56 | 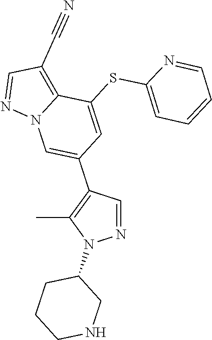 [SMILES: Cc1c(-c2cc(Sc3ccccn3)c3c(C#N)cnn3c2)cnn1[C@H]1CCCNC1] |
| 57 | 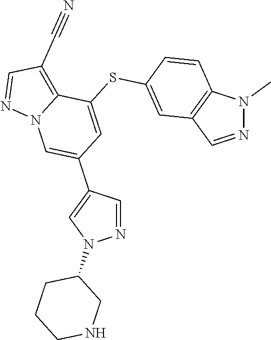 [SMILES: Cn1ncc2cc(Sc3cc(-c4cnn([C@H]5CCCNC5)c4)cn4ncc(C#N)c34)ccc21] |
| 58 | 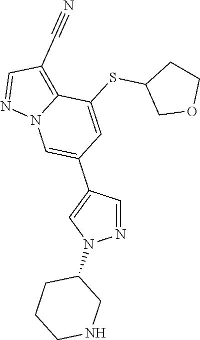 [SMILES: N#Cc1cnn2cc(-c3cnn([C@H]4CCCNC4)c3)cc(SC3CCOC3)c12] |
| 59 | 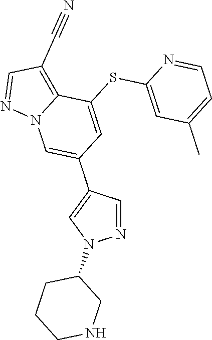 [SMILES: Cc1ccnc(Sc2cc(-c3cnn([C@H]4CCCNC4)c3)cn3ncc(C#N)c23)c1] |
| 60 | 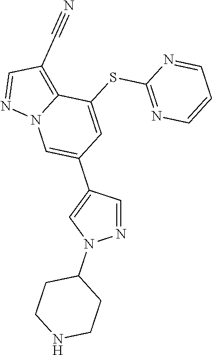 [SMILES: N#Cc1cnn2cc(-c3cnn(C4CCNCC4)c3)cc(Sc3ncccn3)c12] |
| 61 | 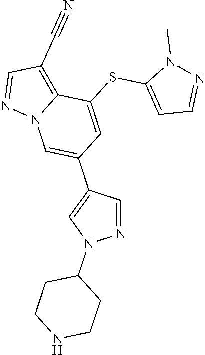 [SMILES: Cn1nccc1Sc1cc(-c2cnn(C3CCNCC3)c2)cn2ncc(C#N)c12] |
| 62 | 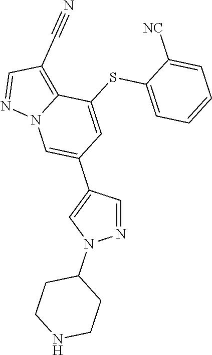 [SMILES: N#Cc1ccccc1Sc1cc(-c2cnn(C3CCNCC3)c2)cn2ncc(C#N)c12] |
| 63 | 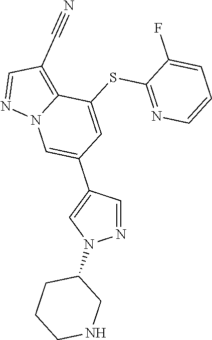 [SMILES: N#Cc1cnn2cc(-c3cnn([C@H]4CCCNC4)c3)cc(Sc3ncccc3F)c12] |
| 64 | 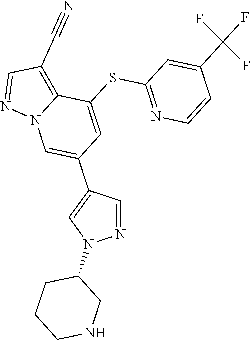 [SMILES: N#Cc1cnn2cc(-c3cnn([C@H]4CCCNC4)c3)cc(Sc3cc(C(F)(F)F)ccn3)c12] |
| 65 | 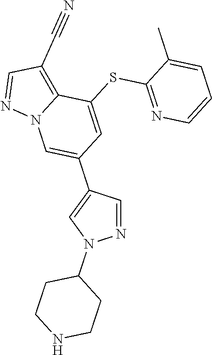 [SMILES: Cc1cccnc1Sc1cc(-c2cnn(C3CCNCC3)c2)cn2ncc(C#N)c12] |
| 66 | 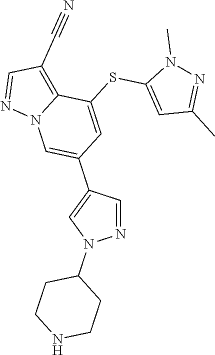 [SMILES: Cc1cc(Sc2cc(-c3cnn(C4CCNCC4)c3)cn3ncc(C#N)c23)n(C)n1] |
| 67 | 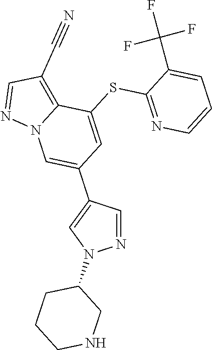 [SMILES: N#Cc1cnn2cc(-c3cnn([C@H]4CCCNC4)c3)cc(Sc3ncccc3C(F)(F)F)c12] |
| 68 | 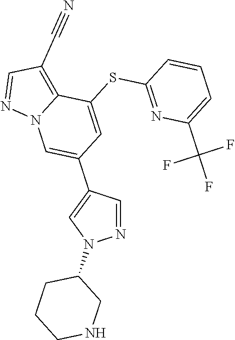 [SMILES: N#Cc1cnn2cc(-c3cnn([C@H]4CCCNC4)c3)cc(Sc3cccc(C(F)(F)F)n3)c12] |
| 69 | 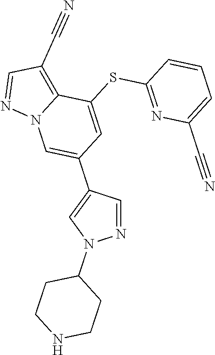 [SMILES: N#Cc1cccc(Sc2cc(-c3cnn(C4CCNCC4)c3)cn3ncc(C#N)c23)n1] |
| 70 | 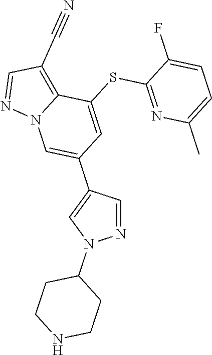 [SMILES: Cc1ccc(F)c(Sc2cc(-c3cnn(C4CCNCC4)c3)cn3ncc(C#N)c23)n1] |
| 71 | 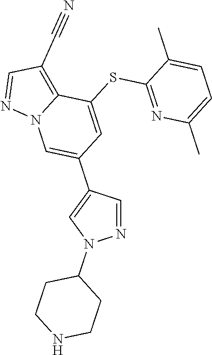 [SMILES: Cc1ccc(C)c(Sc2cc(-c3cnn(C4CCNCC4)c3)cn3ncc(C#N)c23)n1] |
| 72 | 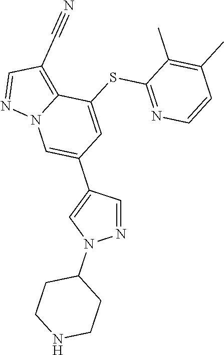 [SMILES: Cc1ccnc(Sc2cc(-c3cnn(C4CCNCC4)c3)cn3ncc(C#N)c23)c1C] |
| 73 | 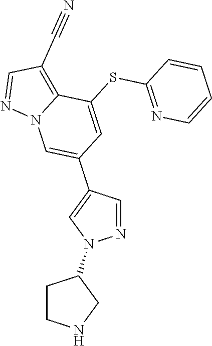 |
| 74 | 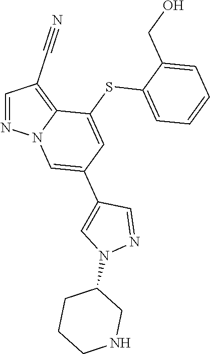 [SMILES: N#Cc1cnn2cc(-c3cnn([C@H]4CCCNC4)c3)cc(Sc3ccccc3CO)c12] |
| 75 | 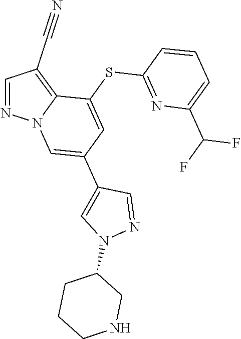 [SMILES: N#Cc1cnn2cc(-c3cnn([C@H]4CCCNC4)c3)cc(Sc3cccc(C(F)F)n3)c12] |
| 76 | 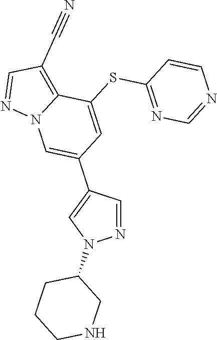 [SMILES: N#Cc1cnn2cc(-c3cnn([C@H]4CCCNC4)c3)cc(Sc3ccncn3)c12] |
| 77 | 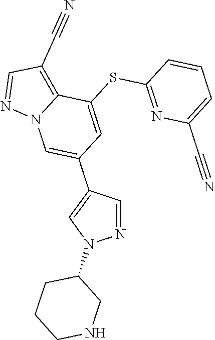 [SMILES: N#Cc1cccc(Sc2cc(-c3cnn([C@H]4CCCNC4)c3)cn3ncc(C#N)c23)n1] |
| 78 | 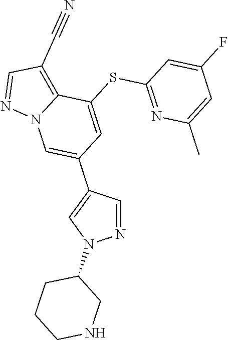 [SMILES: Cc1cc(F)cc(Sc2cc(-c3cnn([C@H]4CCCNC4)c3)cn3ncc(C#N)c23)n1] |
| 79 | 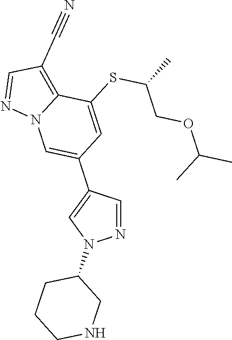 [SMILES: CC(C)OC[C@@H](C)Sc1cc(-c2cnn([C@H]3CCCNC3)c2)cn2ncc(C#N)c12] |
| 80 | 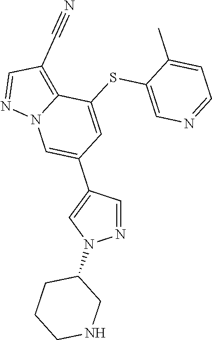 [SMILES: Cc1ccncc1Sc1cc(-c2cnn([C@H]3CCCNC3)c2)cn2ncc(C#N)c12] |
| 81 | 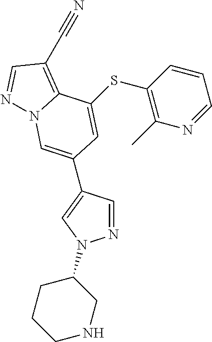 [SMILES: Cc1ncccc1Sc1cc(-c2cnn([C@H]3CCCNC3)c2)cn2ncc(C#N)c12] |
| 82 | 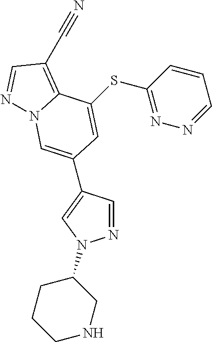 [SMILES: N#Cc1cnn2cc(-c3cnn([C@H]4CCCNC4)c3)cc(Sc3cccnn3)c12] |
| 83 | 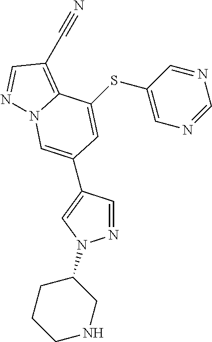 [SMILES: N#Cc1cnn2cc(-c3cnn([C@H]4CCCNC4)c3)cc(Sc3cncnc3)c12] |
| 84 | 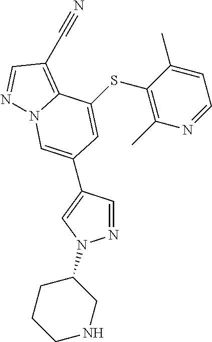 [SMILES: Cc1ccnc(C)c1Sc1cc(-c2cnn([C@H]3CCCNC3)c2)cn2ncc(C#N)c12] |
| 85 | 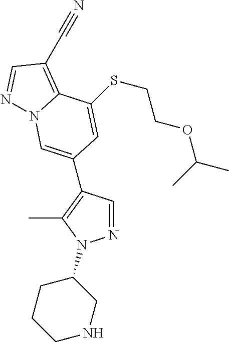 [SMILES: Cc1c(-c2cc(SCCOC(C)C)c3c(C#N)cnn3c2)cnn1[C@H]1CCCNC1] |
| 86 | 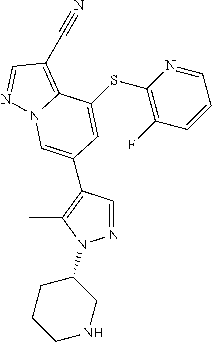 [SMILES: Cc1c(-c2cc(Sc3ncccc3F)c3c(C#N)cnn3c2)cnn1[C@H]1CCCNC1] |
| 87 | 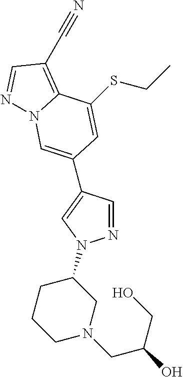 [SMILES: CCSc1cc(-c2cnn([C@H]3CCCN(C[C@H](O)CO)C3)c2)cn2ncc(C#N)c12] |
| 88 | 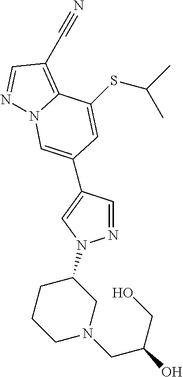 [SMILES: CC(C)Sc1cc(-c2cnn([C@H]3CCCN(C[C@H](O)CO)C3)c2)cn2ncc(C#N)c12] |
| 89 | 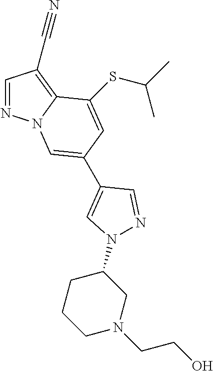 [SMILES: CC(C)Sc1cc(-c2cnn([C@H]3CCCN(CCO)C3)c2)cn2ncc(C#N)c12] |
| 90 | 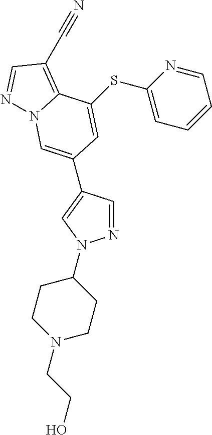 [SMILES: N#Cc1cnn2cc(-c3cnn(C4CCN(CCO)CC4)c3)cc(Sc3ccccn3)c12] |
| 91 | 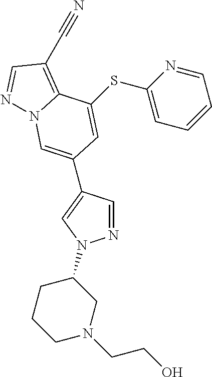 [SMILES: N#Cc1cnn2cc(-c3cnn([C@H]4CCCN(CCO)C4)c3)cc(Sc3ccccn3)c12] |
| 92 | 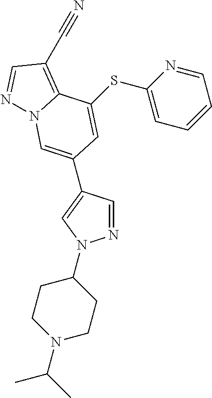 [SMILES: CC(C)N1CCC(n2cc(-c3cc(Sc4ccccn4)c4c(C#N)cnn4c3)cn2)CC1] |
| 93 | 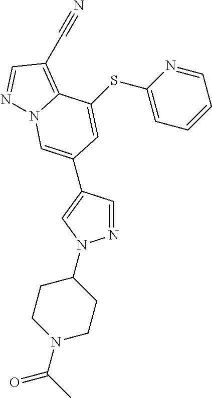 [SMILES: CC(=O)N1CCC(n2cc(-c3cc(Sc4ccccn4)c4c(C#N)cnn4c3)cn2)CC1] |
| 94 | 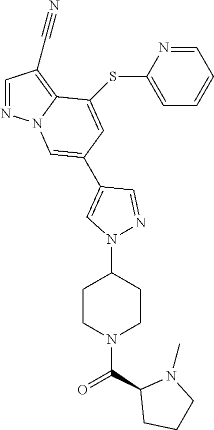 [SMILES: CN1CCC[C@H]1C(=O)N1CCC(n2cc(-c3cc(Sc4ccccn4)c4c(C#N)cnn4c3)cn2)CC1] |
| 95 | 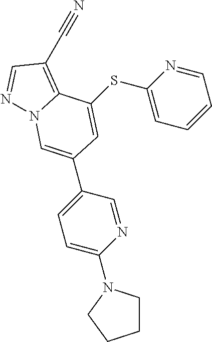 [SMILES: N#Cc1cnn2cc(-c3ccc(N4CCCC4)nc3)cc(Sc3ccccn3)c12] |
| 96 | 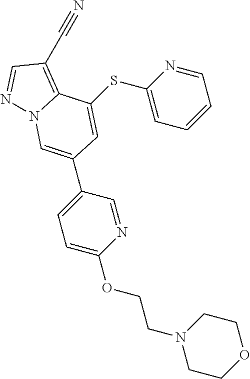 [SMILES: N#Cc1cnn2cc(-c3ccc(OCCN4CCOCC4)nc3)cc(Sc3ccccn3)c12] |
| 97 | 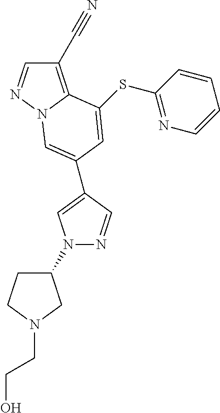 [SMILES: N#Cc1cnn2cc(-c3cnn([C@H]4CCN(CCO)C4)c3)cc(Sc3ccccn3)c12] |
| 98 | 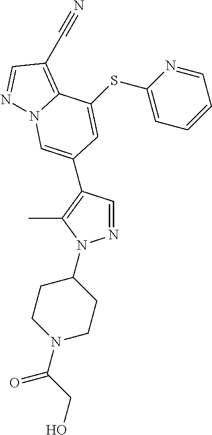 [SMILES: Cc1c(-c2cc(Sc3ccccn3)c3c(C#N)cnn3c2)cnn1C1CCN(C(=O)CO)CC1] |
| 99 | 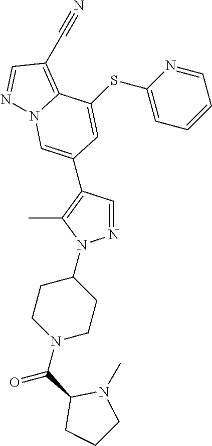 [SMILES: Cc1c(-c2cc(Sc3ccccn3)c3c(C#N)cnn3c2)cnn1C1CCN(C(=O)[C@@H]2CCCN2C)CC1] |
| 100 | 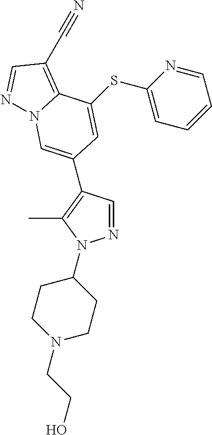 [SMILES: Cc1c(-c2cc(Sc3ccccn3)c3c(C#N)cnn3c2)cnn1C1CCN(CCO)CC1] |
| 101 | 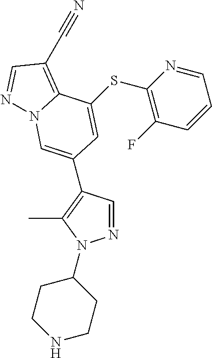 [SMILES: Cc1c(-c2cc(Sc3ncccc3F)c3c(C#N)cnn3c2)cnn1C1CCNCC1] |
| 102 | 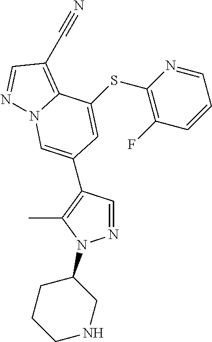 [SMILES: Cc1c(-c2cc(Sc3ncccc3F)c3c(C#N)cnn3c2)cnn1[C@@H]1CCCNC1] |
| 103 | 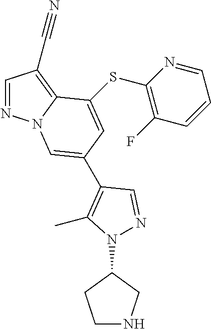 [SMILES: Cc1c(-c2cc(Sc3ncccc3F)c3c(C#N)cnn3c2)cnn1[C@H]1CCNC1] |
| 104 | 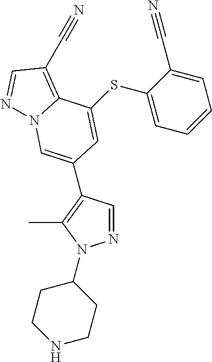 [SMILES: Cc1c(-c2cc(Sc3ccccc3C#N)c3c(C#N)cnn3c2)cnn1C1CCNCC1] |
| 105 | 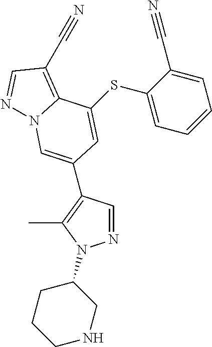 [SMILES: Cc1c(-c2cc(Sc3ccccc3C#N)c3c(C#N)cnn3c2)cnn1[C@H]1CCCNC1] |
| 106 | 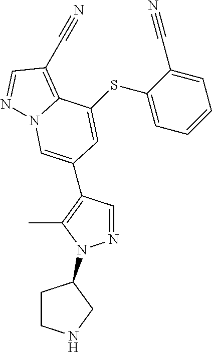 [SMILES: Cc1c(-c2cc(Sc3ccccc3C#N)c3c(C#N)cnn3c2)cnn1[C@@H]1CCNC1] |
| 107 | 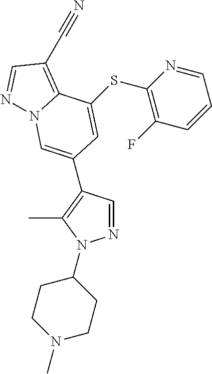 [SMILES: Cc1c(-c2cc(Sc3ncccc3F)c3c(C#N)cnn3c2)cnn1C1CCN(C)CC1] |
| 108 | 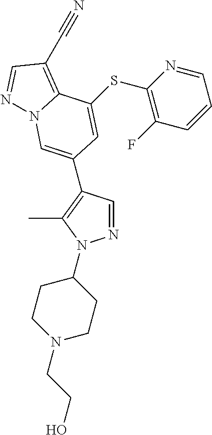 [SMILES: Cc1c(-c2cc(Sc3ncccc3F)c3c(C#N)cnn3c2)cnn1C1CCN(CCO)CC1] |
| 109 | 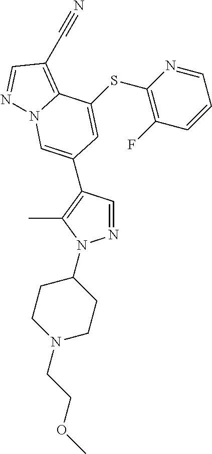 [SMILES: COCCN1CCC(n2ncc(-c3cc(Sc4ncccc4F)c4c(C#N)cnn4c3)c2C)CC1] |
| 110 | 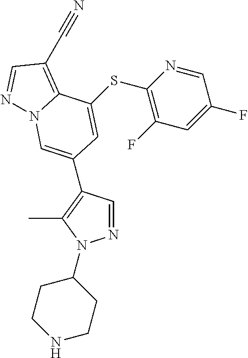 [SMILES: Cc1c(-c2cc(Sc3ncc(F)cc3F)c3c(C#N)cnn3c2)cnn1C1CCNCC1] |
| 111 | 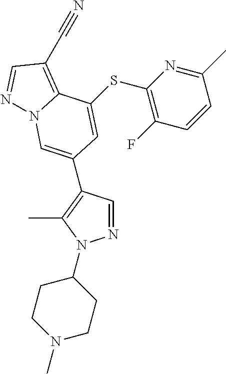 [SMILES: Cc1ccc(F)c(Sc2cc(-c3cnn(C4CCN(C)CC4)c3C)cn3ncc(C#N)c23)n1] |
| 112 | 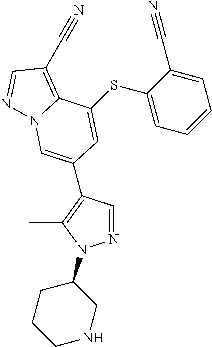 [SMILES: Cc1c(-c2cc(Sc3ccccc3C#N)c3c(C#N)cnn3c2)cnn1[C@@H]1CCCNC1] |
| 113 | 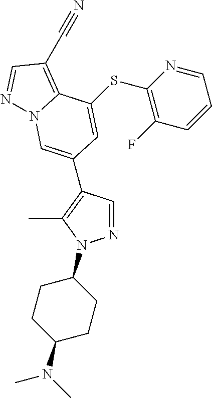 [SMILES: Cc1c(-c2cc(Sc3ncccc3F)c3c(C#N)cnn3c2)cnn1[C@H]1CC[C@@H](N(C)C)CC1] |
| 114 | 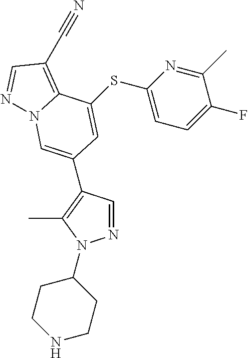 [SMILES: Cc1nc(Sc2cc(-c3cnn(C4CCNCC4)c3C)cn3ncc(C#N)c23)ccc1F] |
| 115 | 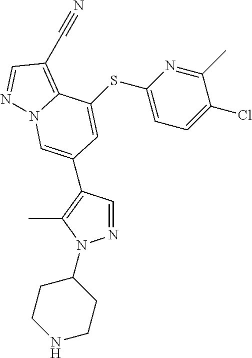 [SMILES: Cc1nc(Sc2cc(-c3cnn(C4CCNCC4)c3C)cn3ncc(C#N)c23)ccc1Cl] |
| 116 | 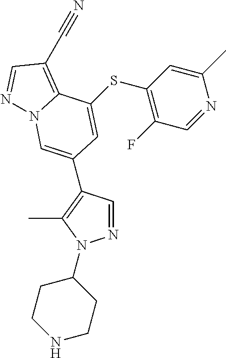 [SMILES: Cc1cc(Sc2cc(-c3cnn(C4CCNCC4)c3C)cn3ncc(C#N)c23)c(F)cn1] |
| 117 | 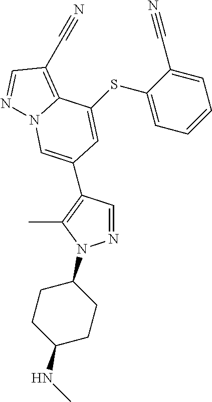 [SMILES: CN[C@H]1CC[C@@H](n2ncc(-c3cc(Sc4ccccc4C#N)c4c(C#N)cnn4c3)c2C)CC1] |
| 118 | 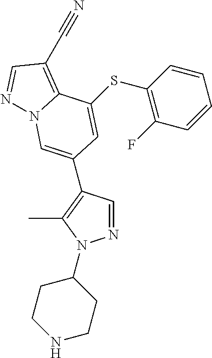 [SMILES: Cc1c(-c2cc(Sc3ccccc3F)c3c(C#N)cnn3c2)cnn1C1CCNCC1] |
| 119 | 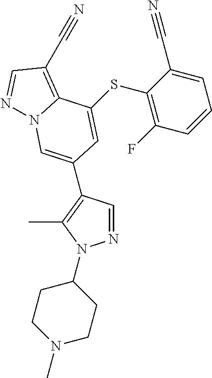 [SMILES: Cc1c(-c2cc(Sc3c(F)cccc3C#N)c3c(C#N)cnn3c2)cnn1C1CCN(C)CC1] |
| 120 | 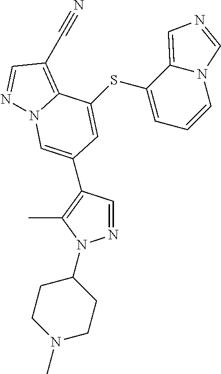 [SMILES: Cc1c(-c2cc(Sc3cccn4cncc34)c3c(C#N)cnn3c2)cnn1C1CCN(C)CC1] |
| 121 | 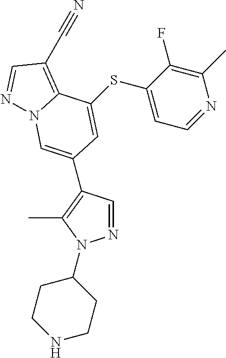 [SMILES: Cc1nccc(Sc2cc(-c3cnn(C4CCNCC4)c3C)cn3ncc(C#N)c23)c1F] |
| 122 | 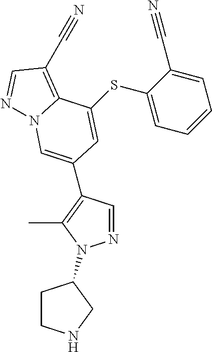 [SMILES: Cc1c(-c2cc(Sc3ccccc3C#N)c3c(C#N)cnn3c2)cnn1[C@H]1CCNC1] |
| 123 | 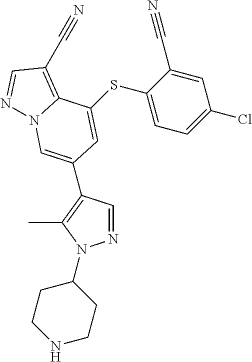 [SMILES: Cc1c(-c2cc(Sc3ccc(Cl)cc3C#N)c3c(C#N)cnn3c2)cnn1C1CCNCC1] |
| 124 | 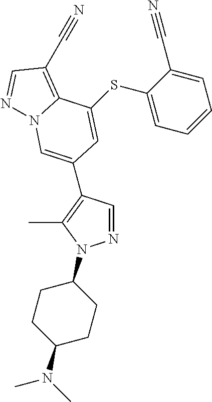 [SMILES: Cc1c(-c2cc(Sc3ccccc3C#N)c3c(C#N)cnn3c2)cnn1[C@H]1CC[C@@H](N(C)C)CC1] |
| 125 | 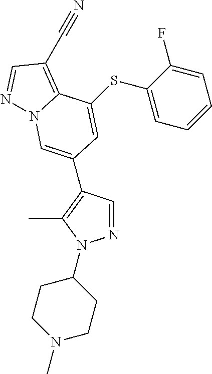 [SMILES: Cc1c(-c2cc(Sc3ccccc3F)c3c(C#N)cnn3c2)cnn1C1CCN(C)CC1] |
| 126 | 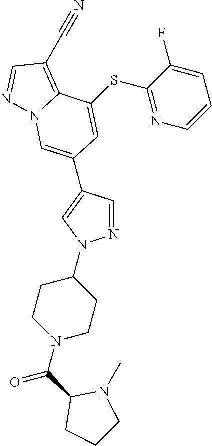 [SMILES: CN1CCC[C@H]1C(=O)N1CCC(n2cc(-c3cc(Sc4ncccc4F)c4c(C#N)cnn4c3)cn2)CC1] |
| 127 | 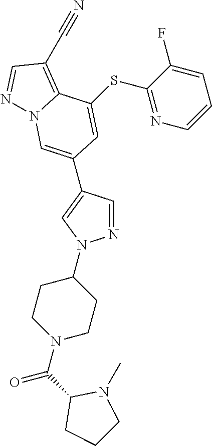 [SMILES: CN1CCC[C@@H]1C(=O)N1CCC(n2cc(-c3cc(Sc4ncccc4F)c4c(C#N)cnn4c3)cn2)CC1] |
| 128 | 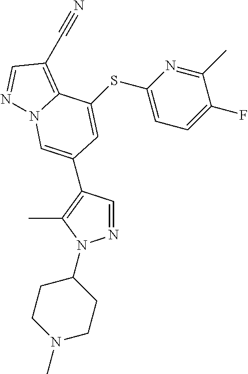 [SMILES: Cc1nc(Sc2cc(-c3cnn(C4CCN(C)CC4)c3C)cn3ncc(C#N)c23)ccc1F] |
| 129 | 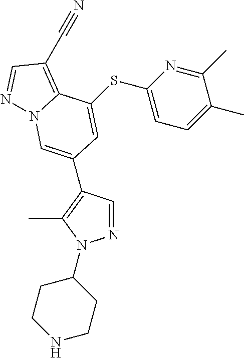 [SMILES: Cc1ccc(Sc2cc(-c3cnn(C4CCNCC4)c3C)cn3ncc(C#N)c23)nc1C] |
| 130 | 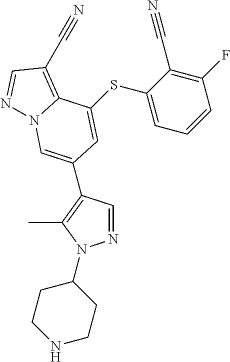 [SMILES: Cc1c(-c2cc(Sc3cccc(F)c3C#N)c3c(C#N)cnn3c2)cnn1C1CCNCC1] |
| 131 | 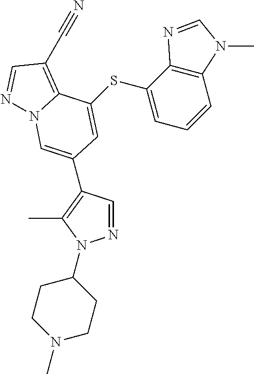 [SMILES: Cc1c(-c2cc(Sc3cccc4c3ncn4C)c3c(C#N)cnn3c2)cnn1C1CCN(C)CC1] |
| 132 | 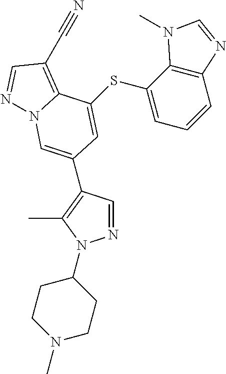 [SMILES: Cc1c(-c2cc(Sc3cccc4ncn(C)c34)c3c(C#N)cnn3c2)cnn1C1CCN(C)CC1] |
| 133 | 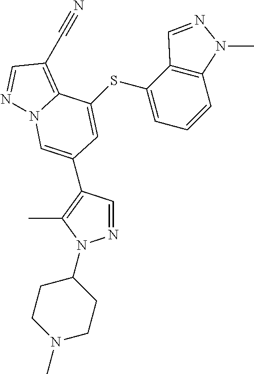 [SMILES: Cc1c(-c2cc(Sc3cccc4c3cnn4C)c3c(C#N)cnn3c2)cnn1C1CCN(C)CC1] |
| 134 | 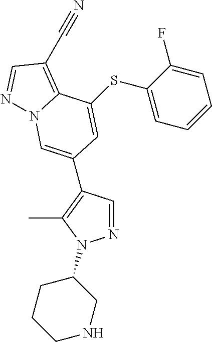 [SMILES: Cc1c(-c2cc(Sc3ccccc3F)c3c(C#N)cnn3c2)cnn1[C@H]1CCCNC1] |
| 135 | 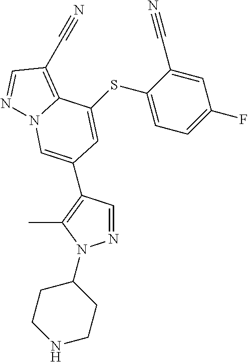 [SMILES: Cc1c(-c2cc(Sc3ccc(F)cc3C#N)c3c(C#N)cnn3c2)cnn1C1CCNCC1] |
| 136 | 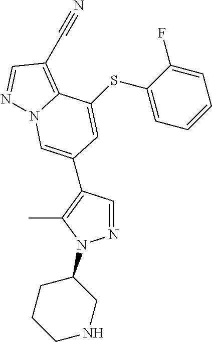 [SMILES: Cc1c(-c2cc(Sc3ccccc3F)c3c(C#N)cnn3c2)cnn1[C@@H]1CCCNC1] |
| 137 | 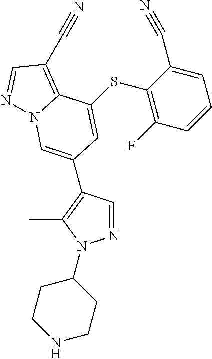 [SMILES: Cc1c(-c2cc(Sc3c(F)cccc3C#N)c3c(C#N)cnn3c2)cnn1C1CCNCC1] |
| 138 | 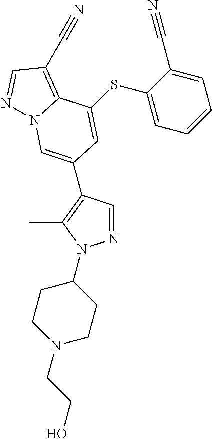 [SMILES: Cc1c(-c2cc(Sc3ccccc3C#N)c3c(C#N)cnn3c2)cnn1C1CCN(CCO)CC1] |
| 139 | 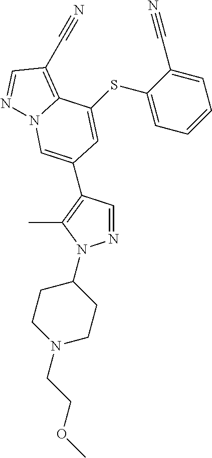 [SMILES: COCCN1CCC(n2ncc(-c3cc(Sc4ccccc4C#N)c4c(C#N)cnn4c3)c2C)CC1] |
| 140 | 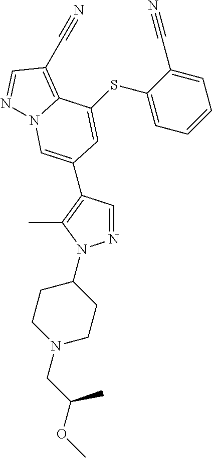 [SMILES: CO[C@H](C)CN1CCC(n2ncc(-c3cc(Sc4ccccc4C#N)c4c(C#N)cnn4c3)c2C)CC1] |
| 141 | 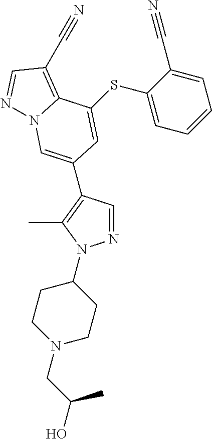 [SMILES: Cc1c(-c2cc(Sc3ccccc3C#N)c3c(C#N)cnn3c2)cnn1C1CCN(C[C@@H](C)O)CC1] |
| 142 | 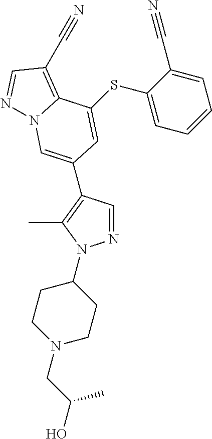 [SMILES: Cc1c(-c2cc(Sc3ccccc3C#N)c3c(C#N)cnn3c2)cnn1C1CCN(C[C@H](C)O)CC1] |
| 143 | 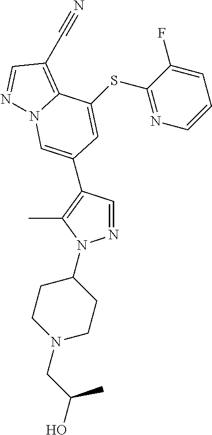 [SMILES: Cc1c(-c2cc(Sc3ncccc3F)c3c(C#N)cnn3c2)cnn1C1CCN(C[C@@H](C)O)CC1] |
| 144 | 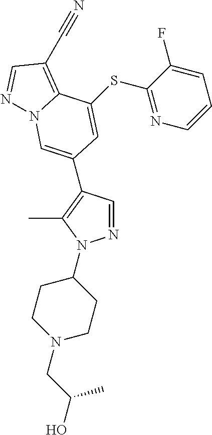 [SMILES: Cc1c(-c2cc(Sc3ncccc3F)c3c(C#N)cnn3c2)cnn1C1CCN(C[C@H](C)O)CC1] |
| 145 | 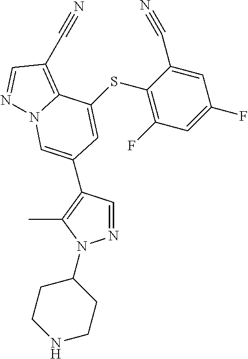 [SMILES: Cc1c(-c2cc(Sc3c(F)cc(F)cc3C#N)c3c(C#N)cnn3c2)cnn1C1CCNCC1] |
| 146 | 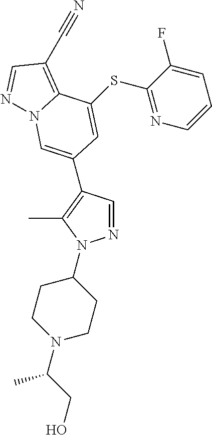 [SMILES: Cc1c(-c2cc(Sc3ncccc3F)c3c(C#N)cnn3c2)cnn1C1CCN([C@@H](C)CO)CC1] |
| 147 | 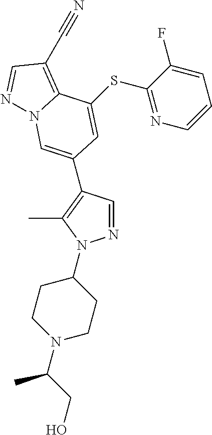 [SMILES: Cc1c(-c2cc(Sc3ncccc3F)c3c(C#N)cnn3c2)cnn1C1CCN([C@H](C)CO)CC1] |
| 148 | 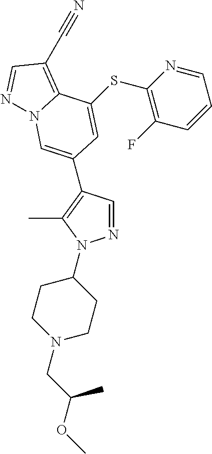 [SMILES: CO[C@H](C)CN1CCC(n2ncc(-c3cc(Sc4ncccc4F)c4c(C#N)cnn4c3)c2C)CC1] |
| 149 | 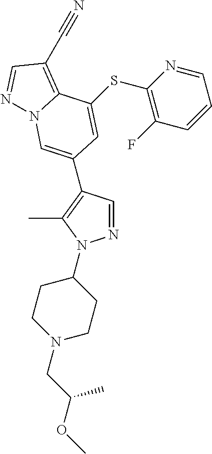 [SMILES: CO[C@@H](C)CN1CCC(n2ncc(-c3cc(Sc4ncccc4F)c4c(C#N)cnn4c3)c2C)CC1] |
| 150 | 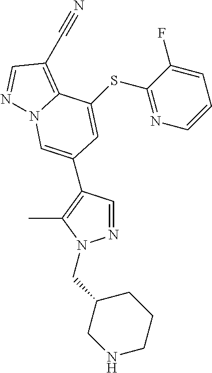 [SMILES: Cc1c(-c2cc(Sc3ncccc3F)c3c(C#N)cnn3c2)cnn1C[C@@H]1CCCNC1] |
| 151 | 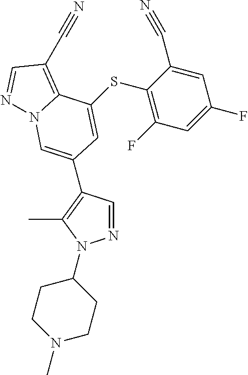 [SMILES: Cc1c(-c2cc(Sc3c(F)cc(F)cc3C#N)c3c(C#N)cnn3c2)cnn1C1CCN(C)CC1] |
| 152 | 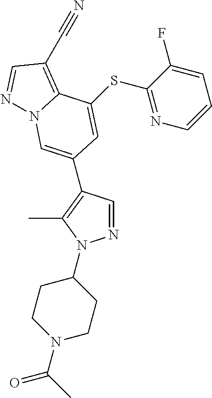 [SMILES: CC(=O)N1CCC(n2ncc(-c3cc(Sc4ncccc4F)c4c(C#N)cnn4c3)c2C)CC1] |
| 153 | 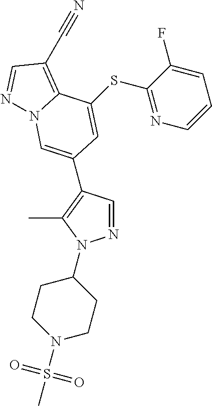 [SMILES: Cc1c(-c2cc(Sc3ncccc3F)c3c(C#N)cnn3c2)cnn1C1CCN(S(C)(=O)=O)CC1] |
| 154 | 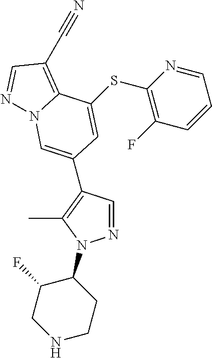 [SMILES: Cc1c(-c2cc(Sc3ncccc3F)c3c(C#N)cnn3c2)cnn1[C@H]1CCNC[C@@H]1F] |
| 155 | 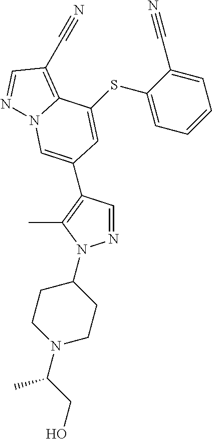 [SMILES: Cc1c(-c2cc(Sc3ccccc3C#N)c3c(C#N)cnn3c2)cnn1C1CCN([C@@H](C)CO)CC1] |
| 156 | 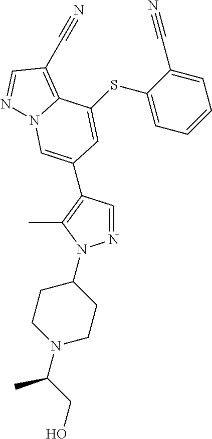 [SMILES: Cc1c(-c2cc(Sc3ccccc3C#N)c3c(C#N)cnn3c2)cnn1C1CCN([C@H](C)CO)CC1] |
| 157 | 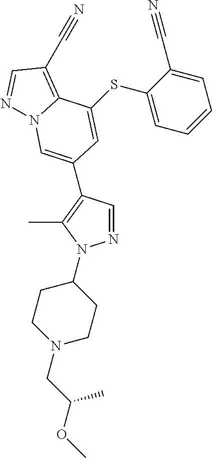 [SMILES: CO[C@@H](C)CN1CCC(n2ncc(-c3cc(Sc4ccccc4C#N)c4c(C#N)cnn4c3)c2C)CC1] |
| 158 | 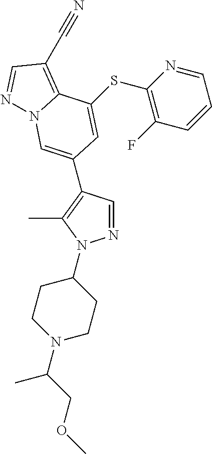 [SMILES: COCC(C)N1CCC(n2ncc(-c3cc(Sc4ncccc4F)c4c(C#N)cnn4c3)c2C)CC1] |
| 159 | 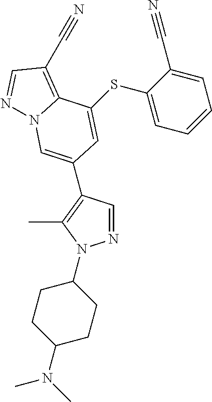 [SMILES: Cc1c(-c2cc(Sc3ccccc3C#N)c3c(C#N)cnn3c2)cnn1C1CCC(N(C)C)CC1] |
| 160 | 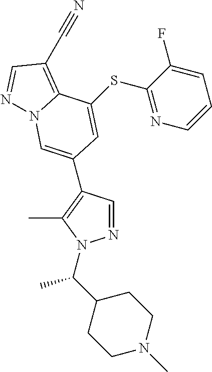 [SMILES: Cc1c(-c2cc(Sc3ncccc3F)c3c(C#N)cnn3c2)cnn1[C@@H](C)C1CCN(C)CC1] |
| 161 | 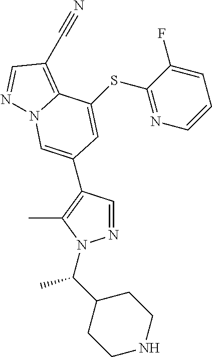 [SMILES: Cc1c(-c2cc(Sc3ncccc3F)c3c(C#N)cnn3c2)cnn1[C@@H](C)C1CCNCC1] |
| 162 | 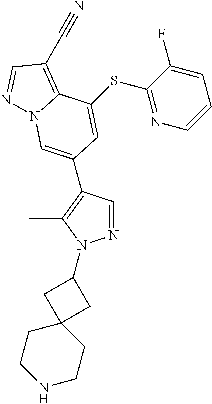 [SMILES: Cc1c(-c2cc(Sc3ncccc3F)c3c(C#N)cnn3c2)cnn1C1CC2(CCNCC2)C1] |
| 163 | 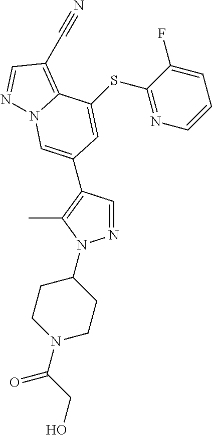 [SMILES: Cc1c(-c2cc(Sc3ncccc3F)c3c(C#N)cnn3c2)cnn1C1CCN(C(=O)CO)CC1] |
| 164 | 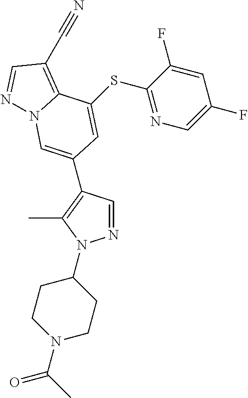 [SMILES: CC(=O)N1CCC(n2ncc(-c3cc(Sc4ncc(F)cc4F)c4c(C#N)cnn4c3)c2C)CC1] |
| 165 | 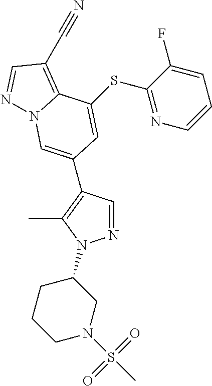 [SMILES: Cc1c(-c2cc(Sc3ncccc3F)c3c(C#N)cnn3c2)cnn1[C@H]1CCCN(S(C)(=O)=O)C1] |
| 166 | 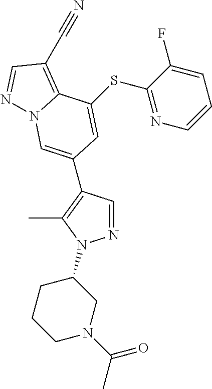 [SMILES: CC(=O)N1CCC[C@H](n2ncc(-c3cc(Sc4ncccc4F)c4c(C#N)cnn4c3)c2C)C1] |
| 167 | 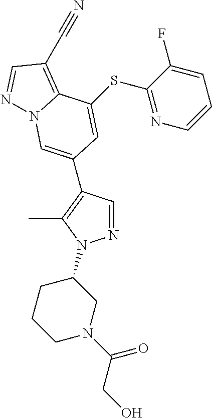 [SMILES: Cc1c(-c2cc(Sc3ncccc3F)c3c(C#N)cnn3c2)cnn1[C@H]1CCCN(C(=O)CO)C1] |
| 168 | 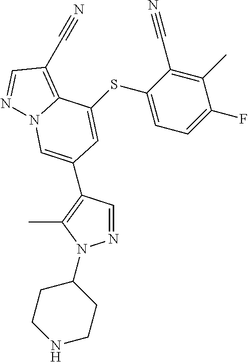 [SMILES: Cc1c(F)ccc(Sc2cc(-c3cnn(C4CCNCC4)c3C)cn3ncc(C#N)c23)c1C#N] |
| 169 | 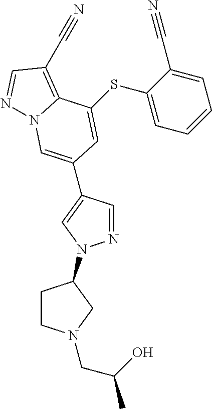 [SMILES: C[C@H](O)CN1CC[C@@H](n2cc(-c3cc(Sc4ccccc4C#N)c4c(C#N)cnn4c3)cn2)C1] |
| 170 | 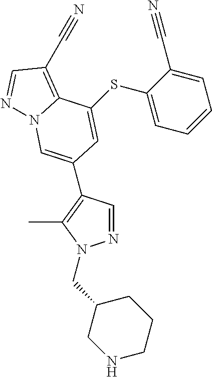 [SMILES: Cc1c(-c2cc(Sc3ccccc3C#N)c3c(C#N)cnn3c2)cnn1C[C@@H]1CCCNC1] |
| 171 | 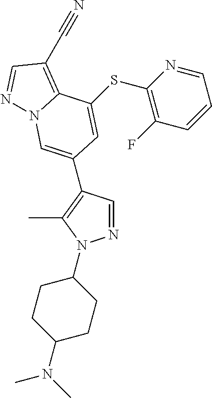 [SMILES: Cc1c(-c2cc(Sc3ncccc3F)c3c(C#N)cnn3c2)cnn1C1CCC(N(C)C)CC1] |
| 172 | 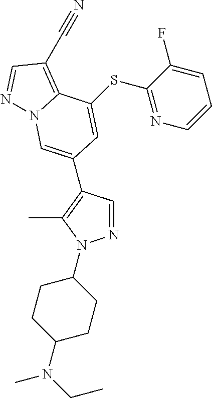 [SMILES: CCN(C)C1CCC(n2ncc(-c3cc(Sc4ncccc4F)c4c(C#N)cnn4c3)c2C)CC1] |
| 173 | 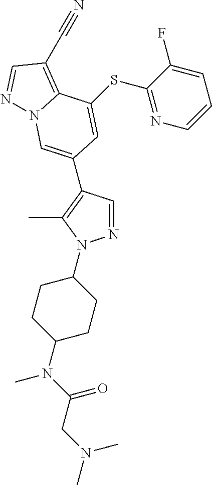 [SMILES: Cc1c(-c2cc(Sc3ncccc3F)c3c(C#N)cnn3c2)cnn1C1CCC(N(C)C(=O)CN(C)C)CC1] |
| 174 | 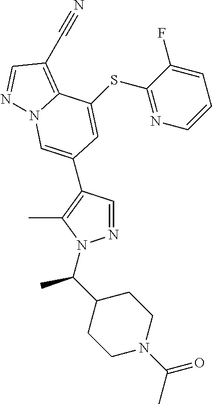 [SMILES: CC(=O)N1CCC([C@@H](C)n2ncc(-c3cc(Sc4ncccc4F)c4c(C#N)cnn4c3)c2C)CC1] |
| 175 | 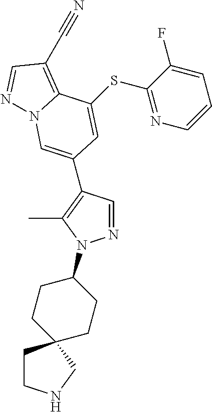 [SMILES: Cc1c(-c2cc(Sc3ncccc3F)c3c(C#N)cnn3c2)cnn1[C@H]1CC[C@@]2(CCNC2)CC1] |
| 176 | 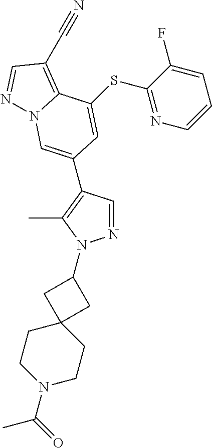 [SMILES: CC(=O)N1CCC2(CC1)CC(n1ncc(-c3cc(Sc4ncccc4F)c4c(C#N)cnn4c3)c1C)C2] |
| 177 | 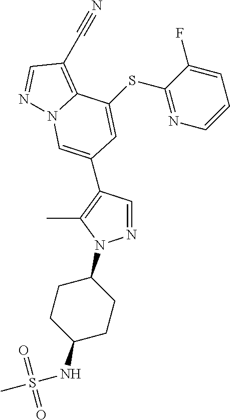 [SMILES: Cc1c(-c2cc(Sc3ncccc3F)c3c(C#N)cnn3c2)cnn1[C@H]1CC[C@@H](NS(C)(=O)=O)CC1] |
| 178 | 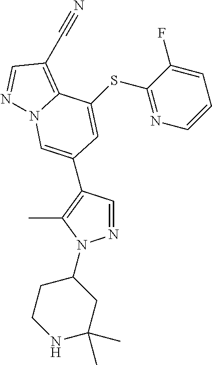 [SMILES: Cc1c(-c2cc(Sc3ncccc3F)c3c(C#N)cnn3c2)cnn1C1CCNC(C)(C)C1] |
| 179 | 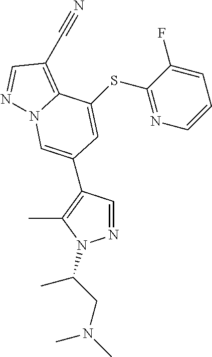 [SMILES: Cc1c(-c2cc(Sc3ncccc3F)c3c(C#N)cnn3c2)cnn1[C@@H](C)CN(C)C] |
| 180 | 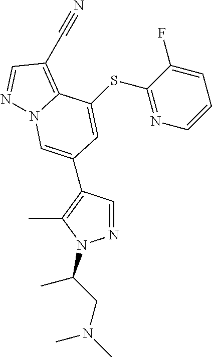 [SMILES: Cc1c(-c2cc(Sc3ncccc3F)c3c(C#N)cnn3c2)cnn1[C@H](C)CN(C)C] |
| 181 | 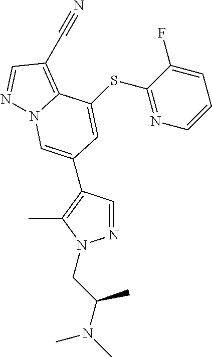 [SMILES: Cc1c(-c2cc(Sc3ncccc3F)c3c(C#N)cnn3c2)cnn1C[C@@H](C)N(C)C] |
| 182 | 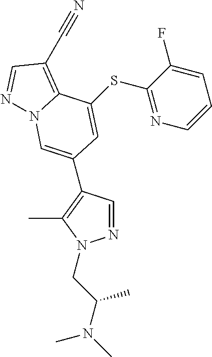 [SMILES: Cc1c(-c2cc(Sc3ncccc3F)c3c(C#N)cnn3c2)cnn1C[C@H](C)N(C)C] |
| 183 | 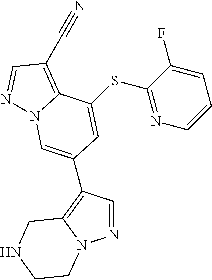 [SMILES: N#Cc1cnn2cc(-c3cnn4c3CNCC4)cc(Sc3ncccc3F)c12] |
| 184 | 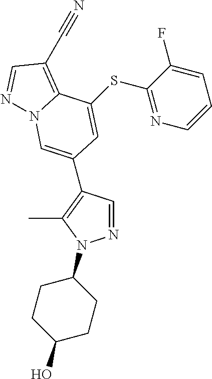 [SMILES: Cc1c(-c2cc(Sc3ncccc3F)c3c(C#N)cnn3c2)cnn1[C@H]1CC[C@@H](O)CC1] |
| 185 | 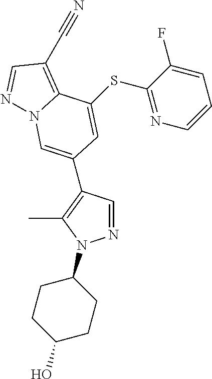 [SMILES: Cc1c(-c2cc(Sc3ncccc3F)c3c(C#N)cnn3c2)cnn1[C@H]1CC[C@H](O)CC1] |
| 186 | 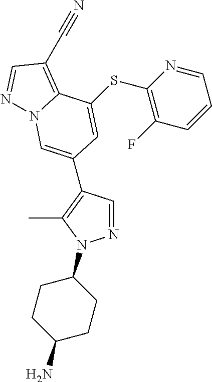 [SMILES: Cc1c(-c2cc(Sc3ncccc3F)c3c(C#N)cnn3c2)cnn1[C@H]1CC[C@@H](N)CC1] |
| 187 | 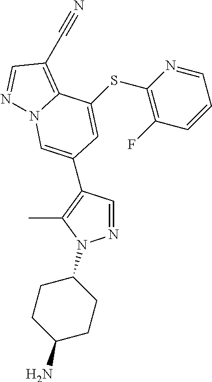 [SMILES: Cc1c(-c2cc(Sc3ncccc3F)c3c(C#N)cnn3c2)cnn1[C@H]1CC[C@H](N)CC1] |
| 188 | 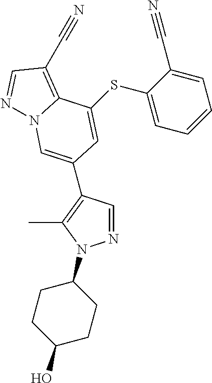 [SMILES: Cc1c(-c2cc(Sc3ccccc3C#N)c3c(C#N)cnn3c2)cnn1[C@H]1CC[C@@H](O)CC1] |
| 189 | 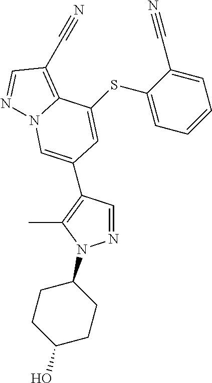 [SMILES: Cc1c(-c2cc(Sc3ccccc3C#N)c3c(C#N)cnn3c2)cnn1[C@H]1CC[C@H](O)CC1] |
| 190 | 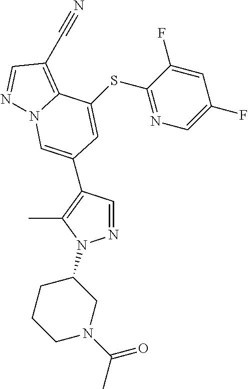 [SMILES: CC(=O)N1CCC[C@H](n2ncc(-c3cc(Sc4ncc(F)cc4F)c4c(C#N)cnn4c3)c2C)C1] |
| 191 | 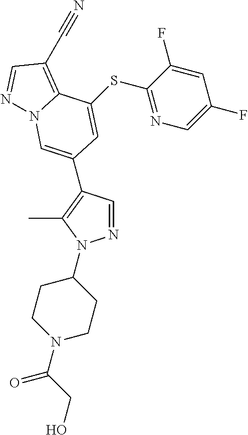 [SMILES: Cc1c(-c2cc(Sc3ncc(F)cc3F)c3c(C#N)cnn3c2)cnn1C1CCN(C(=O)CO)CC1] |
| 192 | 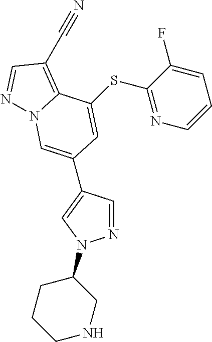 [SMILES: N#Cc1cnn2cc(-c3cnn([C@@H]4CCCNC4)c3)cc(Sc3ncccc3F)c12] |
| 193 | 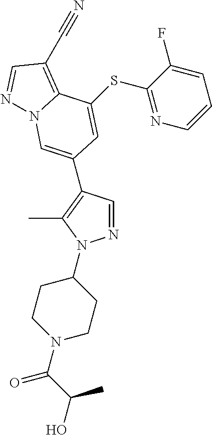 [SMILES: Cc1c(-c2cc(Sc3ncccc3F)c3c(C#N)cnn3c2)cnn1C1CCN(C(=O)[C@@H](C)O)CC1] |
| 194 | 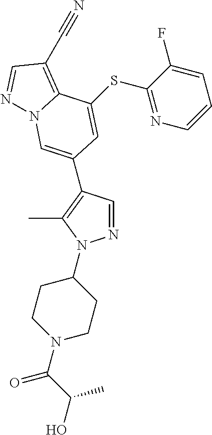 [SMILES: Cc1c(-c2cc(Sc3ncccc3F)c3c(C#N)cnn3c2)cnn1C1CCN(C(=O)[C@H](C)O)CC1] |
| 195 | 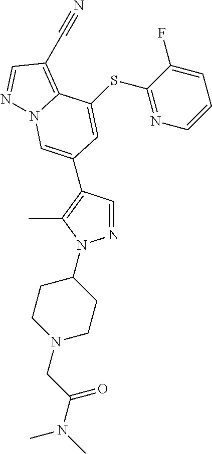 [SMILES: Cc1c(-c2cc(Sc3ncccc3F)c3c(C#N)cnn3c2)cnn1C1CCN(CC(=O)N(C)C)CC1] |
| 196 | 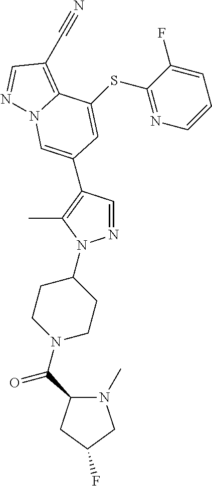 [SMILES: Cc1c(-c2cc(Sc3ncccc3F)c3c(C#N)cnn3c2)cnn1C1CCN(C(=O)[C@@H]2C[C@@H](F)CN2C)CC1] |
| 197 | 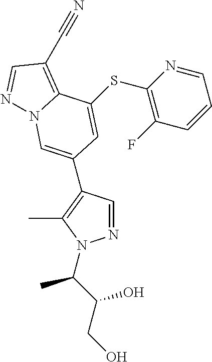 [SMILES: Cc1c(-c2cc(Sc3ncccc3F)c3c(C#N)cnn3c2)cnn1[C@H](C)[C@H](O)CO] |
| 198 | 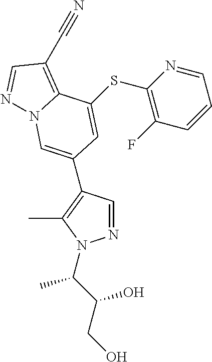 [SMILES: Cc1c(-c2cc(Sc3ncccc3F)c3c(C#N)cnn3c2)cnn1[C@@H](C)[C@H](O)CO] |
| 199 | 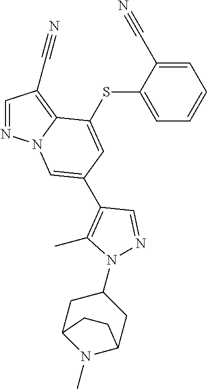 [SMILES: Cc1c(-c2cc(Sc3ccccc3C#N)c3c(C#N)cnn3c2)cnn1C1CC2CCC(C1)N2C] |
| 200 | 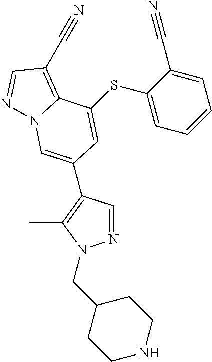 [SMILES: Cc1c(-c2cc(Sc3ccccc3C#N)c3c(C#N)cnn3c2)cnn1CC1CCNCC1] |
| 201 | 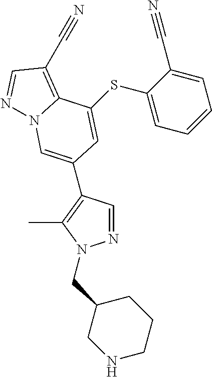 [SMILES: Cc1c(-c2cc(Sc3ccccc3C#N)c3c(C#N)cnn3c2)cnn1C[C@H]1CCCNC1] |
| 202 | 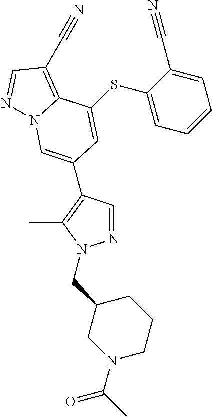 [SMILES: CC(=O)N1CCC[C@H](Cn2ncc(-c3cc(Sc4ccccc4C#N)c4c(C#N)cnn4c3)c2C)C1] |
| 203 | 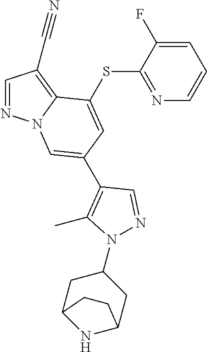 [SMILES: Cc1c(-c2cc(Sc3ncccc3F)c3c(C#N)cnn3c2)cnn1C1CC2CCC(C1)N2] |
| 204 | 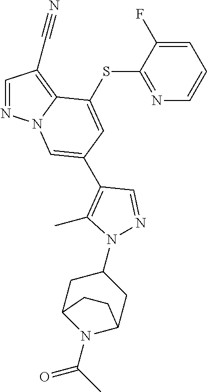 [SMILES: CC(=O)N1C2CCC1CC(n1ncc(-c3cc(Sc4ncccc4F)c4c(C#N)cnn4c3)c1C)C2] |
| 205 | 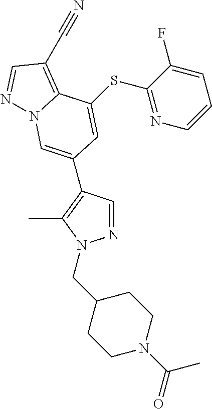 [SMILES: CC(=O)N1CCC(Cn2ncc(-c3cc(Sc4ncccc4F)c4c(C#N)cnn4c3)c2C)CC1] |
| 206 | 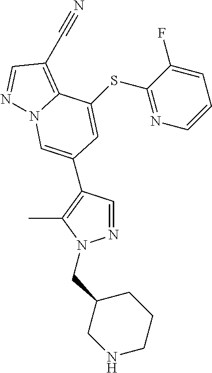 [SMILES: Cc1c(-c2cc(Sc3ncccc3F)c3c(C#N)cnn3c2)cnn1C[C@H]1CCCNC1] |
| 207 | 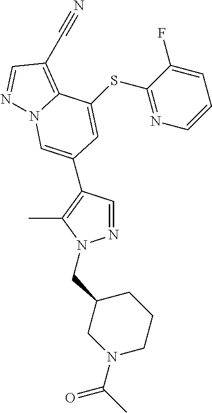 [SMILES: CC(=O)N1CCC[C@H](Cn2ncc(-c3cc(Sc4ncccc4F)c4c(C#N)cnn4c3)c2C)C1] |
| 208 | 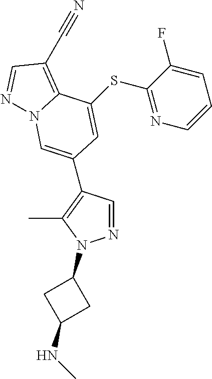 [SMILES: CN[C@H]1C[C@@H](n2ncc(-c3cc(Sc4ncccc4F)c4c(C#N)cnn4c3)c2C)C1] |
| 209 | 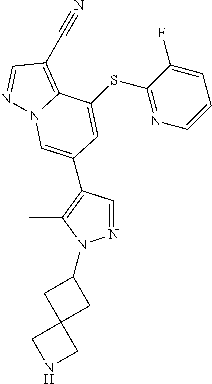 [SMILES: Cc1c(-c2cc(Sc3ncccc3F)c3c(C#N)cnn3c2)cnn1C1CC2(CNC2)C1] |
| 210 | 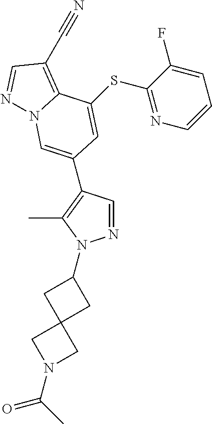 [SMILES: CC(=O)N1CC2(CC(n3ncc(-c4cc(Sc5ncccc5F)c5c(C#N)cnn5c4)c3C)C2)C1] |
| 211 | 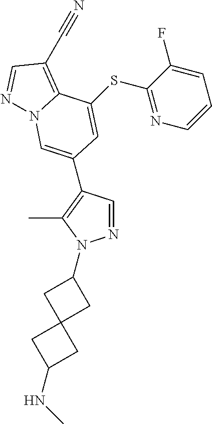 [SMILES: CNC1CC2(C1)CC(n1ncc(-c3cc(Sc4ncccc4F)c4c(C#N)cnn4c3)c1C)C2] |
| 212 | 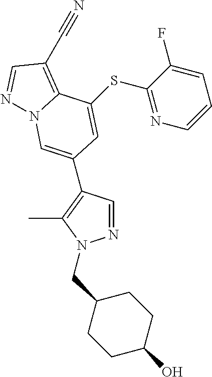 [SMILES: Cc1c(-c2cc(Sc3ncccc3F)c3c(C#N)cnn3c2)cnn1C[C@H]1CC[C@@H](O)CC1] |
| 213 | 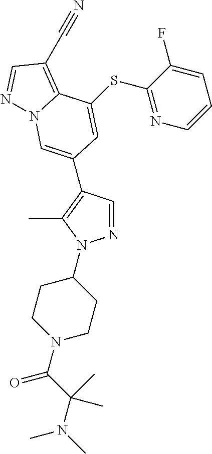 [SMILES: Cc1c(-c2cc(Sc3ncccc3F)c3c(C#N)cnn3c2)cnn1C1CCN(C(=O)C(C)(C)N(C)C)CC1] |
| 214 | 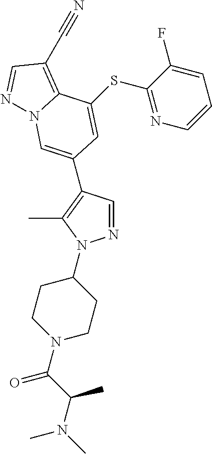 [SMILES: Cc1c(-c2cc(Sc3ncccc3F)c3c(C#N)cnn3c2)cnn1C1CCN(C(=O)[C@@H](C)N(C)C)CC1] |
| 215 | 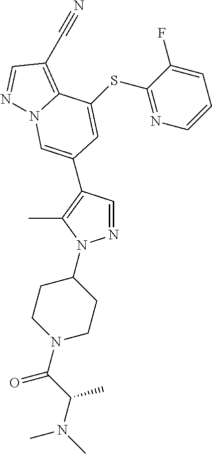 [SMILES: Cc1c(-c2cc(Sc3ncccc3F)c3c(C#N)cnn3c2)cnn1C1CCN(C(=O)[C@H](C)N(C)C)CC1] |
| 216 | 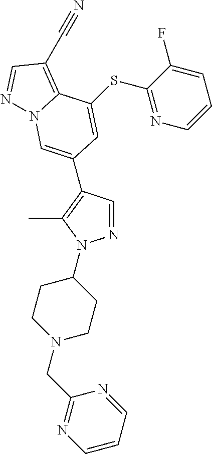 [SMILES: Cc1c(-c2cc(Sc3ncccc3F)c3c(C#N)cnn3c2)cnn1C1CCN(Cc2ncccn2)CC1] |
| 217 | 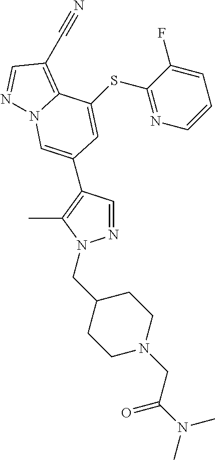 [SMILES: Cc1c(-c2cc(Sc3ncccc3F)c3c(C#N)cnn3c2)cnn1CC1CCN(CC(=O)N(C)C)CC1] |
| 218 | 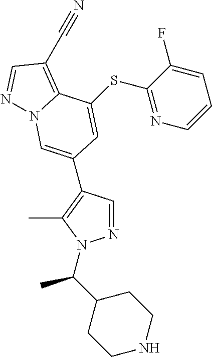 [SMILES: Cc1c(-c2cc(Sc3ncccc3F)c3c(C#N)cnn3c2)cnn1[C@H](C)C1CCNCC1] |
| 219 | 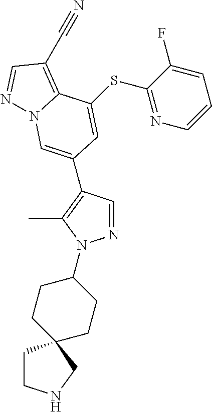 |
| 220 | 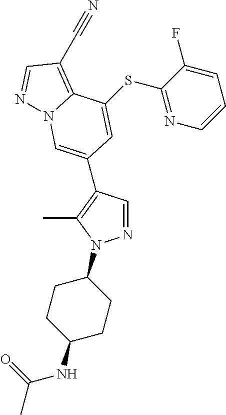 [SMILES: CC(=O)N[C@H]1CC[C@@H](n2ncc(-c3cc(Sc4ncccc4F)c4c(C#N)cnn4c3)c2C)CC1] |
| 221 | 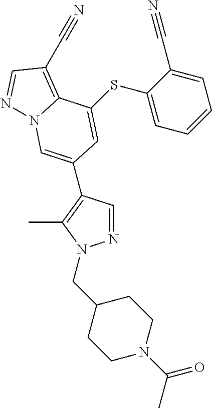 [SMILES: CC(=O)N1CCC(Cn2ncc(-c3cc(Sc4ccccc4C#N)c4c(C#N)cnn4c3)c2C)CC1] |
| 222 | 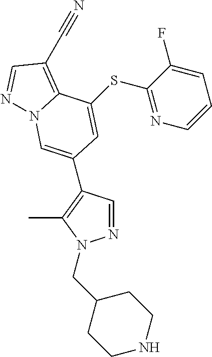 [SMILES: Cc1c(-c2cc(Sc3ncccc3F)c3c(C#N)cnn3c2)cnn1CC1CCNCC1] |
| 223 | 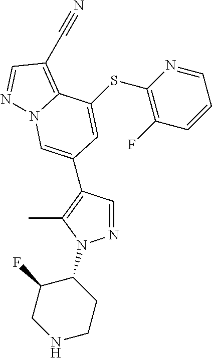 [SMILES: Cc1c(-c2cc(Sc3ncccc3F)c3c(C#N)cnn3c2)cnn1[C@@H]1CCNC[C@H]1F] |
| 224 | 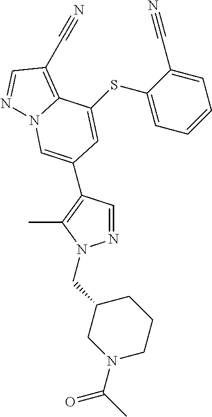 [SMILES: CC(=O)N1CCC[C@@H](Cn2ncc(-c3cc(Sc4ccccc4C#N)c4c(C#N)cnn4c3)c2C)C1] |
| 225 | 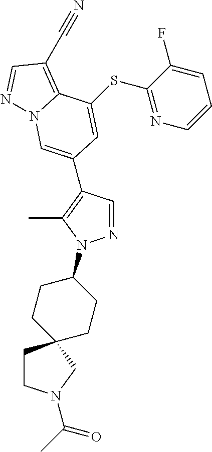 [SMILES: CC(=O)N1CC[C@]2(CC[C@@H](n3ncc(-c4cc(Sc5ncccc5F)c5c(C#N)cnn5c4)c3C)CC2)C1] |
| 226 | 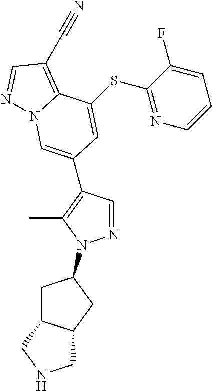 [SMILES: Cc1c(-c2cc(Sc3ncccc3F)c3c(C#N)cnn3c2)cnn1[C@@H]1C[C@H]2CNC[C@H]2C1] |
| 227 | 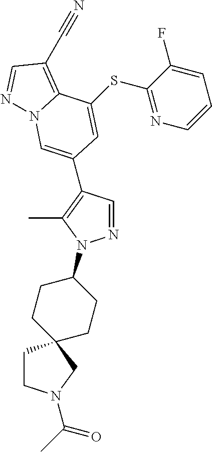 [SMILES: CC(=O)N1CC[C@]2(CC[C@H](n3ncc(-c4cc(Sc5ncccc5F)c5c(C#N)cnn5c4)c3C)CC2)C1] |
| 228 | 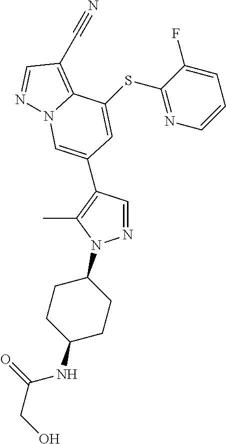 [SMILES: Cc1c(-c2cc(Sc3ncccc3F)c3c(C#N)cnn3c2)cnn1[C@H]1CC[C@@H](NC(=O)CO)CC1] |
| 229 | 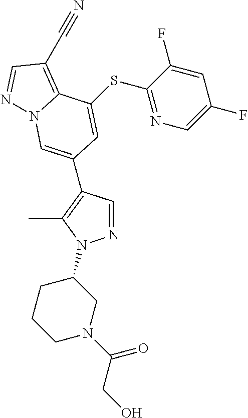 [SMILES: Cc1c(-c2cc(Sc3ncc(F)cc3F)c3c(C#N)cnn3c2)cnn1[C@H]1CCCN(C(=O)CO)C1] |
| 230 | 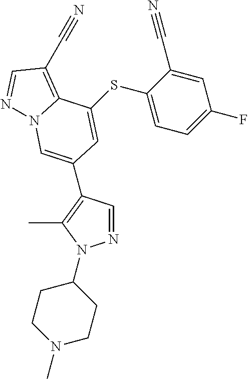 [SMILES: Cc1c(-c2cc(Sc3ccc(F)cc3C#N)c3c(C#N)cnn3c2)cnn1C1CCN(C)CC1] |
| 231 | 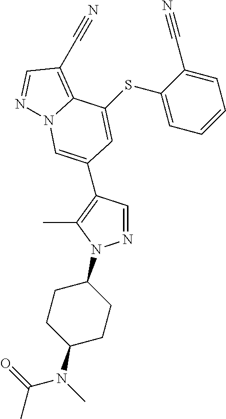 [SMILES: CC(=O)N(C)[C@H]1CC[C@@H](n2ncc(-c3cc(Sc4ccccc4C#N)c4c(C#N)cnn4c3)c2C)CC1] |
| 232 | 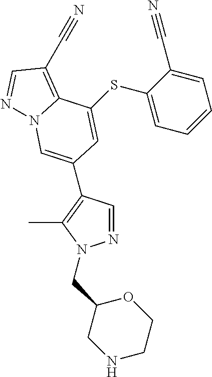 [SMILES: Cc1c(-c2cc(Sc3ccccc3C#N)c3c(C#N)cnn3c2)cnn1C[C@@H]1CNCCO1] |
| 233 | 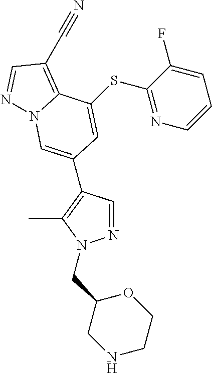 [SMILES: Cc1c(-c2cc(Sc3ncccc3F)c3c(C#N)cnn3c2)cnn1C[C@@H]1CNCCO1] |
| 234 | 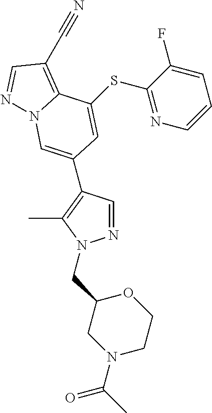 [SMILES: CC(=O)N1CCO[C@H](Cn2ncc(-c3cc(Sc4ncccc4F)c4c(C#N)cnn4c3)c2C)C1] |
| 235 | 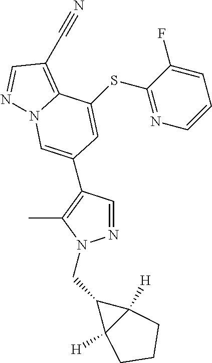 [SMILES: Cc1c(-c2cc(Sc3ncccc3F)c3c(C#N)cnn3c2)cnn1C[C@H]1[C@@H]2CCC[C@@H]21] |
| 236 | 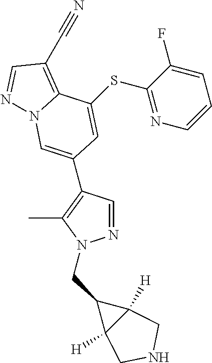 [SMILES: Cc1c(-c2cc(Sc3ncccc3F)c3c(C#N)cnn3c2)cnn1C[C@@H]1[C@@H]2CNC[C@@H]21] |
| 237 | 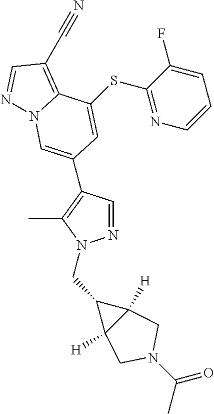 [SMILES: CC(=O)N1C[C@@H]2[C@H](C1)[C@H]2Cn1ncc(-c2cc(Sc3ncccc3F)c3c(C#N)cnn3c2)c1C] |
| 238 | 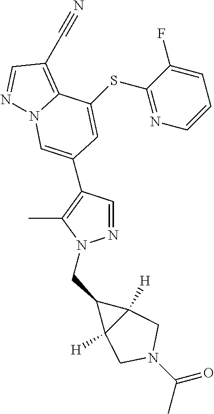 [SMILES: CC(=O)N1C[C@@H]2[C@H](C1)[C@@H]2Cn1ncc(-c2cc(Sc3ncccc3F)c3c(C#N)cnn3c2)c1C] |
| 239 | 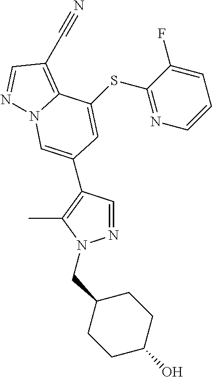 [SMILES: Cc1c(-c2cc(Sc3ncccc3F)c3c(C#N)cnn3c2)cnn1C[C@H]1CC[C@H](O)CC1] |
| 240 | 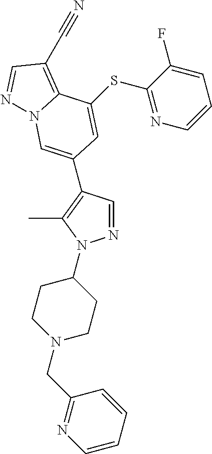 [SMILES: Cc1c(-c2cc(Sc3ncccc3F)c3c(C#N)cnn3c2)cnn1C1CCN(Cc2ccccn2)CC1] |
| 241 | 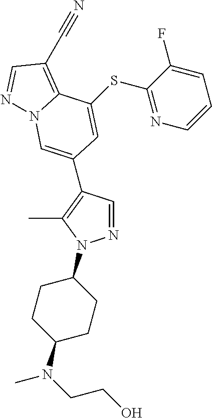 [SMILES: Cc1c(-c2cc(Sc3ncccc3F)c3c(C#N)cnn3c2)cnn1[C@H]1CC[C@@H](N(C)CCO)CC1] |
| 242 | 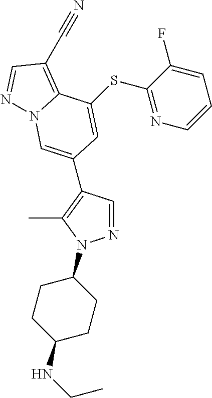 [SMILES: CCN[C@H]1CC[C@@H](n2ncc(-c3cc(Sc4ncccc4F)c4c(C#N)cnn4c3)c2C)CC1] |
| 243 | 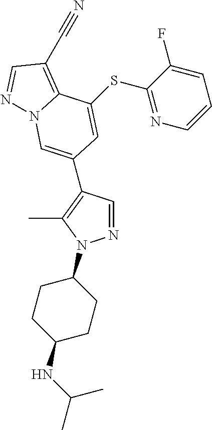 [SMILES: Cc1c(-c2cc(Sc3ncccc3F)c3c(C#N)cnn3c2)cnn1[C@H]1CC[C@@H](NC(C)C)CC1] |
| 244 | 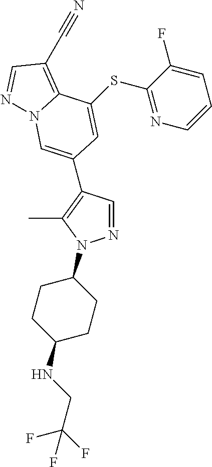 [SMILES: Cc1c(-c2cc(Sc3ncccc3F)c3c(C#N)cnn3c2)cnn1[C@H]1CC[C@@H](NCC(F)(F)F)CC1] |
| 245 | 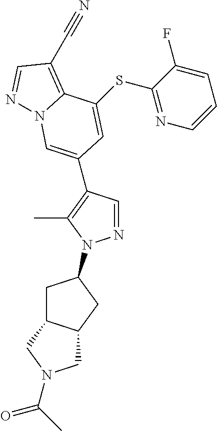 [SMILES: CC(=O)N1C[C@H]2C[C@H](n3ncc(-c4cc(Sc5ncccc5F)c5c(C#N)cnn5c4)c3C)C[C@H]2C1] |
| 246 | 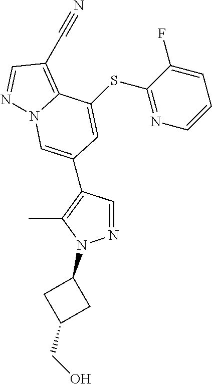 [SMILES: Cc1c(-c2cc(Sc3ncccc3F)c3c(C#N)cnn3c2)cnn1[C@H]1C[C@H](CO)C1] |
| 247 | 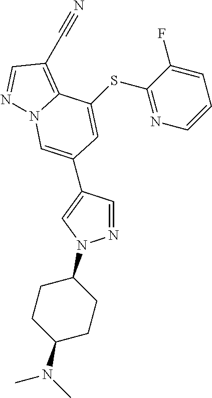 [SMILES: CN(C)[C@H]1CC[C@@H](n2cc(-c3cc(Sc4ncccc4F)c4c(C#N)cnn4c3)cn2)CC1] |
| 248 | 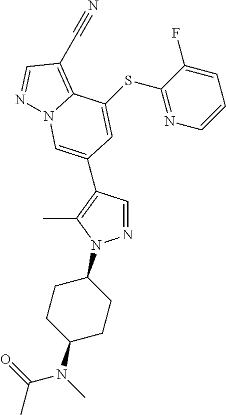 [SMILES: CC(=O)N(C)[C@H]1CC[C@@H](n2ncc(-c3cc(Sc4ncccc4F)c4c(C#N)cnn4c3)c2C)CC1] |
| 249 | 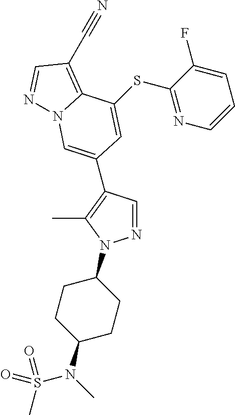 [SMILES: Cc1c(-c2cc(Sc3ncccc3F)c3c(C#N)cnn3c2)cnn1[C@H]1CC[C@@H](N(C)S(C)(=O)=O)CC1] |
| 250 | 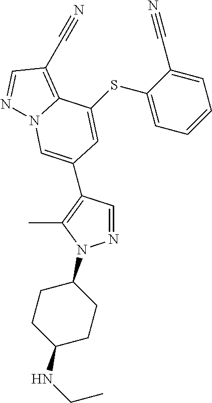 [SMILES: CCN[C@H]1CC[C@@H](n2ncc(-c3cc(Sc4ccccc4C#N)c4c(C#N)cnn4c3)c2C)CC1] |
| 251 | 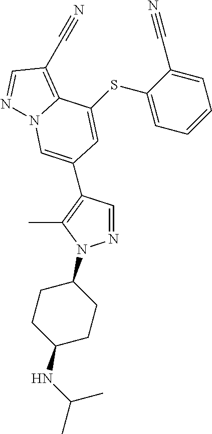 [SMILES: Cc1c(-c2cc(Sc3ccccc3C#N)c3c(C#N)cnn3c2)cnn1[C@H]1CC[C@@H](NC(C)C)CC1] |
| 252 | 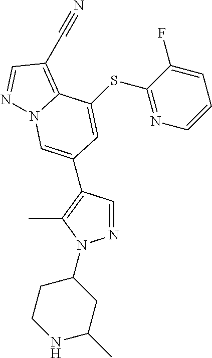 [SMILES: Cc1c(-c2cc(Sc3ncccc3F)c3c(C#N)cnn3c2)cnn1C1CCNC(C)C1] |
| 253 | 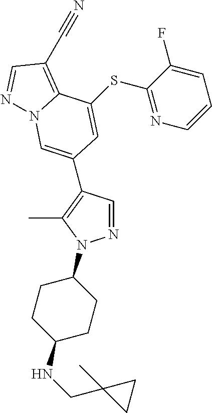 [SMILES: Cc1c(-c2cc(Sc3ncccc3F)c3c(C#N)cnn3c2)cnn1[C@H]1CC[C@@H](NCC2(C)CC2)CC1] |
| 254 | 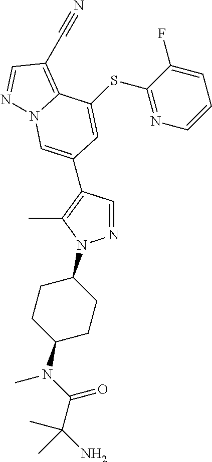 [SMILES: Cc1c(-c2cc(Sc3ncccc3F)c3c(C#N)cnn3c2)cnn1[C@H]1CC[C@@H](N(C)C(=O)C(C)(C)N)CC1] |
| 255 | 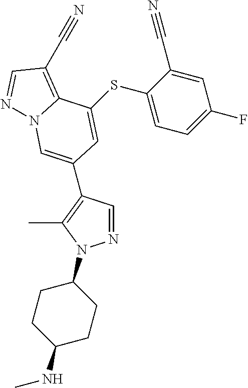 [SMILES: CN[C@H]1CC[C@@H](n2ncc(-c3cc(Sc4ccc(F)cc4C#N)c4c(C#N)cnn4c3)c2C)CC1] |
| 256 | 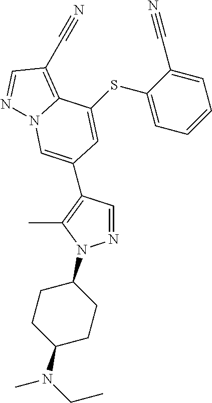 [SMILES: CCN(C)[C@H]1CC[C@@H](n2ncc(-c3cc(Sc4ccccc4C#N)c4c(C#N)cnn4c3)c2C)CC1] |
| 257 | 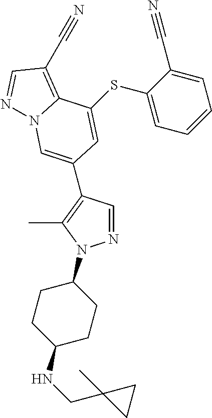 [SMILES: Cc1c(-c2cc(Sc3ccccc3C#N)c3c(C#N)cnn3c2)cnn1[C@H]1CC[C@@H](NCC2(C)CC2)CC1] |
| 258 | 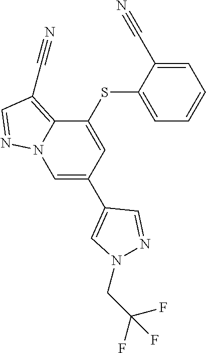 [SMILES: N#Cc1ccccc1Sc1cc(-c2cnn(CC(F)(F)F)c2)cn2ncc(C#N)c12] |
| 259 | 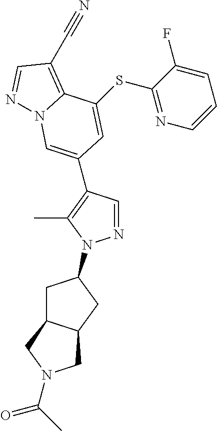 [SMILES: CC(=O)N1C[C@H]2C[C@@H](n3ncc(-c4cc(Sc5ncccc5F)c5c(C#N)cnn5c4)c3C)C[C@H]2C1] |
| 260 | 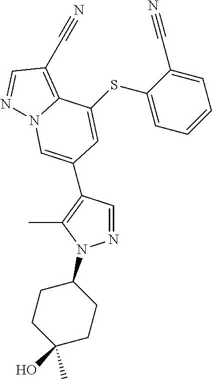 [SMILES: Cc1c(-c2cc(Sc3ccccc3C#N)c3c(C#N)cnn3c2)cnn1[C@H]1CC[C@@](C)(O)CC1] |
| 261 | 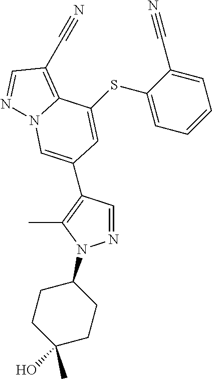 [SMILES: Cc1c(-c2cc(Sc3ccccc3C#N)c3c(C#N)cnn3c2)cnn1[C@H]1CC[C@](C)(O)CC1] |
| 262 | 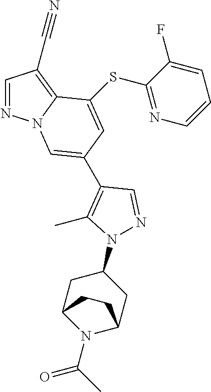 [SMILES: CC(=O)N1[C@@H]2CC[C@H]1C[C@@H](n1ncc(-c3cc(Sc4ncccc4F)c4c(C#N)cnn4c3)c1C)C2] |
| 263 | 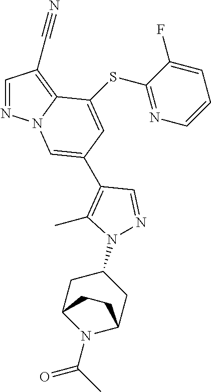 [SMILES: CC(=O)N1[C@@H]2CC[C@H]1C[C@H](n1ncc(-c3cc(Sc4ncccc4F)c4c(C#N)cnn4c3)c1C)C2] |
| 264 | 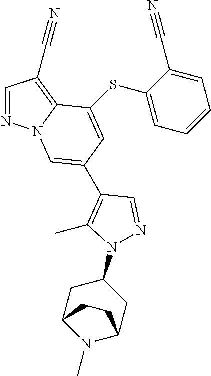 [SMILES: Cc1c(-c2cc(Sc3ccccc3C#N)c3c(C#N)cnn3c2)cnn1[C@H]1C[C@H]2CC[C@@H](C1)N2C] |
| 265 | 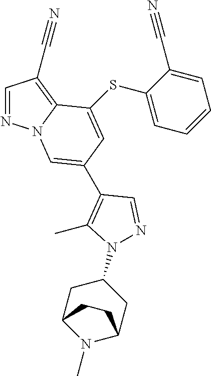 [SMILES: Cc1c(-c2cc(Sc3ccccc3C#N)c3c(C#N)cnn3c2)cnn1[C@@H]1C[C@H]2CC[C@@H](C1)N2C] |
| 266 | 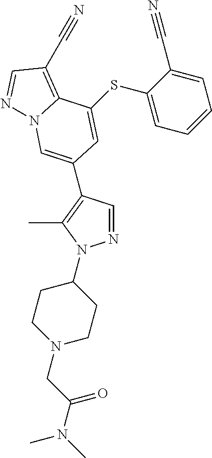 [SMILES: Cc1c(-c2cc(Sc3ccccc3C#N)c3c(C#N)cnn3c2)cnn1C1CCN(CC(=O)N(C)C)CC1] |
| 267 | 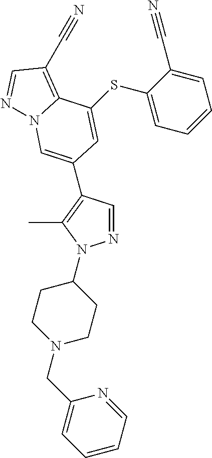 [SMILES: Cc1c(-c2cc(Sc3ccccc3C#N)c3c(C#N)cnn3c2)cnn1C1CCN(Cc2ccccn2)CC1] |
| 268 | 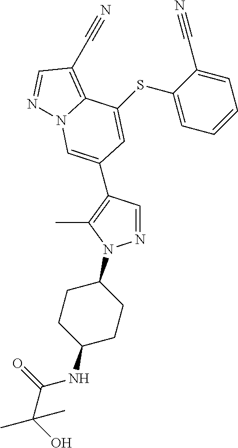 [SMILES: Cc1c(-c2cc(Sc3ccccc3C#N)c3c(C#N)cnn3c2)cnn1[C@H]1CC[C@@H](NC(=O)C(C)(C)O)CC1] |
| 269 | 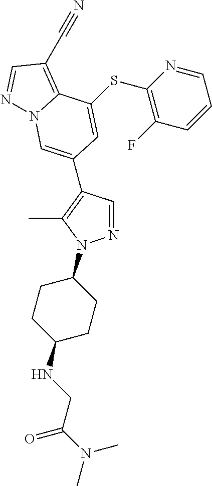 [SMILES: Cc1c(-c2cc(Sc3ncccc3F)c3c(C#N)cnn3c2)cnn1[C@H]1CC[C@@H](NCC(=O)N(C)C)CC1] |
| 270 | 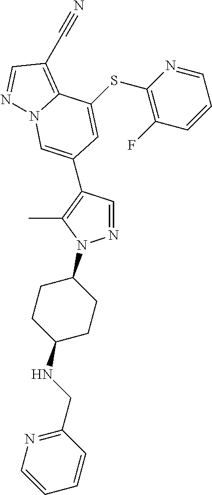 [SMILES: Cc1c(-c2cc(Sc3ncccc3F)c3c(C#N)cnn3c2)cnn1[C@H]1CC[C@@H](NCc2ccccn2)CC1] |
| 271 | 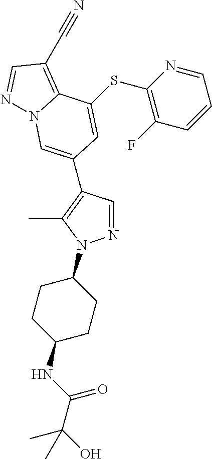 [SMILES: Cc1c(-c2cc(Sc3ncccc3F)c3c(C#N)cnn3c2)cnn1[C@H]1CC[C@@H](NC(=O)C(C)(C)O)CC1] |
| 272 | 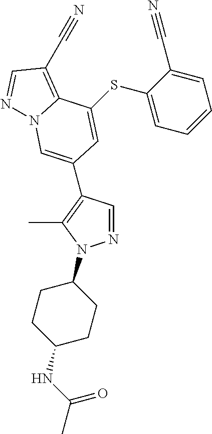 [SMILES: CC(=O)N[C@H]1CC[C@H](n2ncc(-c3cc(Sc4ccccc4C#N)c4c(C#N)cnn4c3)c2C)CC1] |
| 273 | 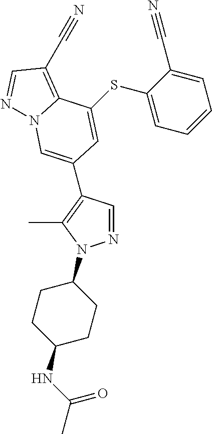 [SMILES: CC(=O)N[C@H]1CC[C@@H](n2ncc(-c3cc(Sc4ccccc4C#N)c4c(C#N)cnn4c3)c2C)CC1] |
| 274 | 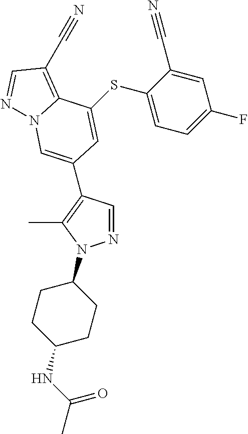 [SMILES: CC(=O)N[C@H]1CC[C@H](n2ncc(-c3cc(Sc4ccc(F)cc4C#N)c4c(C#N)cnn4c3)c2C)CC1] |
| 275 | 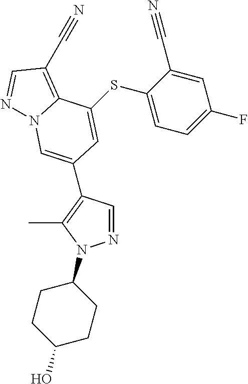 [SMILES: Cc1c(-c2cc(Sc3ccc(F)cc3C#N)c3c(C#N)cnn3c2)cnn1[C@H]1CC[C@H](O)CC1] |
| 276 | 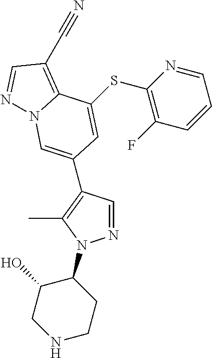 [SMILES: Cc1c(-c2cc(Sc3ncccc3F)c3c(C#N)cnn3c2)cnn1[C@H]1CCNC[C@@H]1O] |
| 277 | 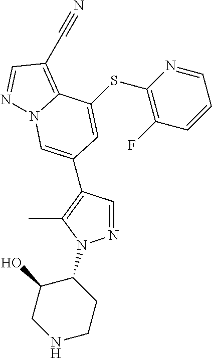 [SMILES: Cc1c(-c2cc(Sc3ncccc3F)c3c(C#N)cnn3c2)cnn1[C@@H]1CCNC[C@H]1O] |
| 278 | 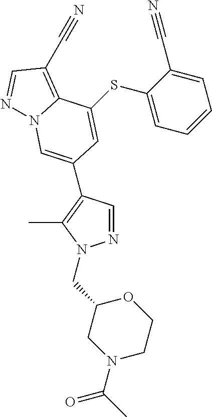 [SMILES: CC(=O)N1CCO[C@@H](Cn2ncc(-c3cc(Sc4ccccc4C#N)c4c(C#N)cnn4c3)c2C)C1] |
| 279 | 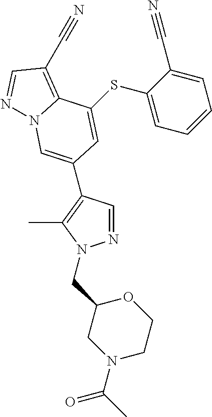 [SMILES: CC(=O)N1CCO[C@H](Cn2ncc(-c3cc(Sc4ccccc4C#N)c4c(C#N)cnn4c3)c2C)C1] |
| 280 | 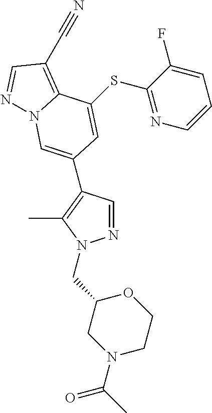 [SMILES: CC(=O)N1CCO[C@@H](Cn2ncc(-c3cc(Sc4ncccc4F)c4c(C#N)cnn4c3)c2C)C1] |
| 281 | 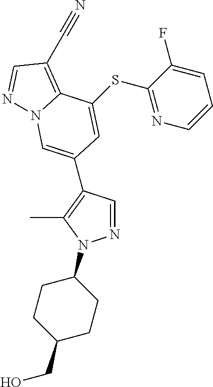 [SMILES: Cc1c(-c2cc(Sc3ncccc3F)c3c(C#N)cnn3c2)cnn1[C@H]1CC[C@@H](CO)CC1] |
| 282 | 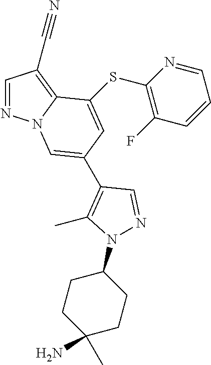 [SMILES: Cc1c(-c2cc(Sc3ncccc3F)c3c(C#N)cnn3c2)cnn1[C@H]1CC[C@@](C)(N)CC1] |
| 283 | 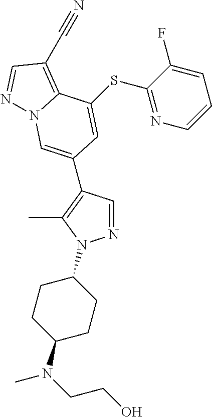 [SMILES: Cc1c(-c2cc(Sc3ncccc3F)c3c(C#N)cnn3c2)cnn1[C@H]1CC[C@H](N(C)CCO)CC1] |
| 284 | 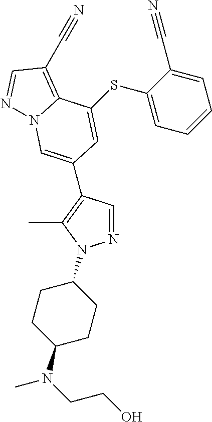 [SMILES: Cc1c(-c2cc(Sc3ccccc3C#N)c3c(C#N)cnn3c2)cnn1[C@H]1CC[C@H](N(C)CCO)CC1] |
| 285 | 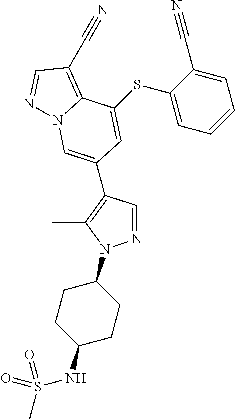 [SMILES: Cc1c(-c2cc(Sc3ccccc3C#N)c3c(C#N)cnn3c2)cnn1[C@H]1CC[C@@H](NS(C)(=O)=O)CC1] |
| 286 | 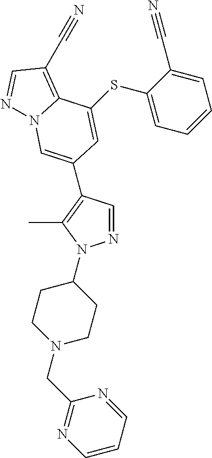 [SMILES: Cc1c(-c2cc(Sc3ccccc3C#N)c3c(C#N)cnn3c2)cnn1C1CCN(Cc2ncccn2)CC1] |
| 287 | 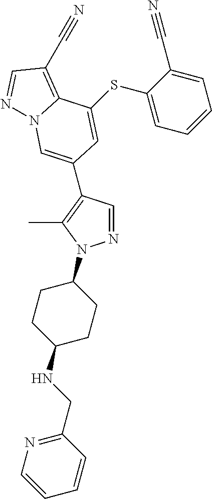 [SMILES: Cc1c(-c2cc(Sc3ccccc3C#N)c3c(C#N)cnn3c2)cnn1[C@H]1CC[C@@H](NCc2ccccn2)CC1] |
| 288 | 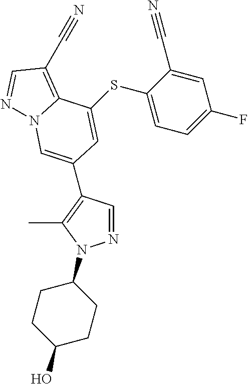 [SMILES: Cc1c(-c2cc(Sc3ccc(F)cc3C#N)c3c(C#N)cnn3c2)cnn1[C@H]1CC[C@@H](O)CC1] |
| 289 | 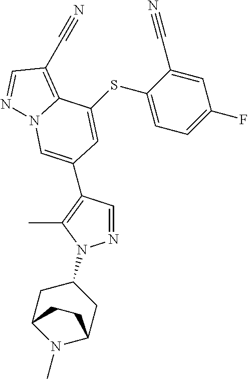 [SMILES: Cc1c(-c2cc(Sc3ccc(F)cc3C#N)c3c(C#N)cnn3c2)cnn1[C@@H]1C[C@H]2CC[C@@H](C1)N2C] |
| 290 | 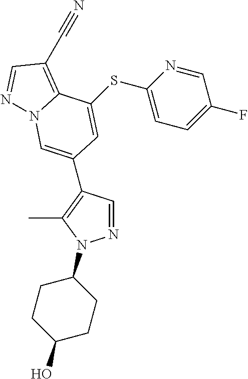 [SMILES: Cc1c(-c2cc(Sc3ccc(F)cn3)c3c(C#N)cnn3c2)cnn1[C@H]1CC[C@@H](O)CC1] |
| 291 | 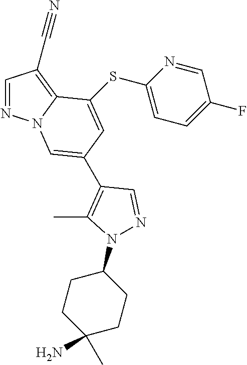 [SMILES: Cc1c(-c2cc(Sc3ccc(F)cn3)c3c(C#N)cnn3c2)cnn1[C@H]1CC[C@@](C)(N)CC1] |
| 292 | 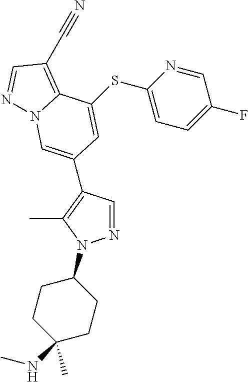 [SMILES: CN[C@]1(C)CC[C@H](n2ncc(-c3cc(Sc4ccc(F)cn4)c4c(C#N)cnn4c3)c2C)CC1] |
| 293 | 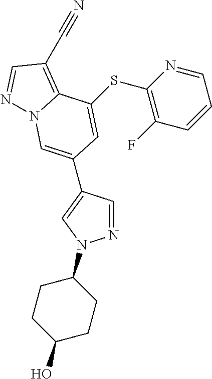 [SMILES: N#Cc1cnn2cc(-c3cnn([C@H]4CC[C@@H](O)CC4)c3)cc(Sc3ncccc3F)c12] |
| 294 | 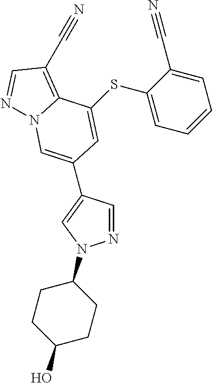 [SMILES: N#Cc1ccccc1Sc1cc(-c2cnn([C@H]3CC[C@@H](O)CC3)c2)cn2ncc(C#N)c12] |
| 295 | 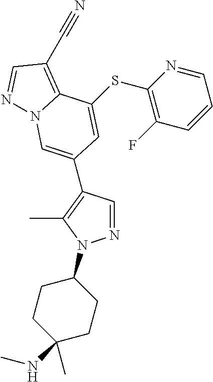 [SMILES: CN[C@]1(C)CC[C@H](n2ncc(-c3cc(Sc4ncccc4F)c4c(C#N)cnn4c3)c2C)CC1] |
| 296 | 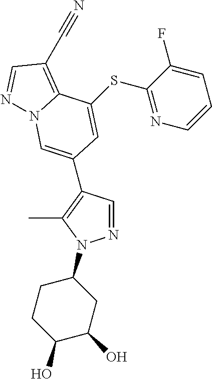 [SMILES: Cc1c(-c2cc(Sc3ncccc3F)c3c(C#N)cnn3c2)cnn1[C@@H]1CC[C@H](O)[C@H](O)C1] |
| 297 | 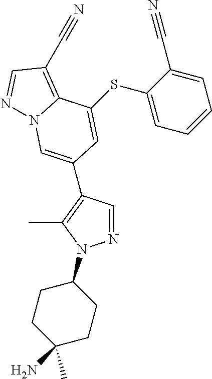 [SMILES: Cc1c(-c2cc(Sc3ccccc3C#N)c3c(C#N)cnn3c2)cnn1[C@H]1CC[C@@](C)(N)CC1] |
| 298 | 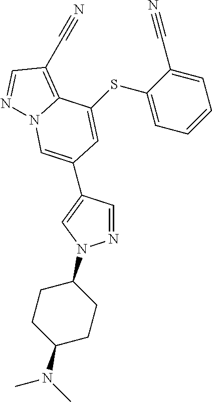 [SMILES: CN(C)[C@H]1CC[C@@H](n2cc(-c3cc(Sc4ccccc4C#N)c4c(C#N)cnn4c3)cn2)CC1] |
| 299 | 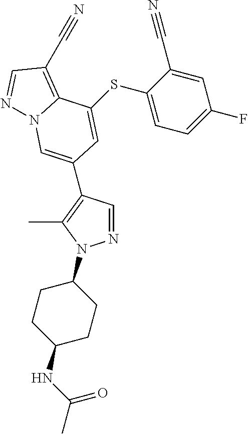 [SMILES: CC(=O)N[C@H]1CC[C@@H](n2ncc(-c3cc(Sc4ccc(F)cc4C#N)c4c(C#N)cnn4c3)c2C)CC1] |
| 300 | 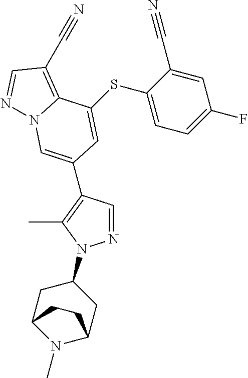 [SMILES: Cc1c(-c2cc(Sc3ccc(F)cc3C#N)c3c(C#N)cnn3c2)cnn1[C@H]1C[C@H]2CC[C@@H](C1)N2C] |
| 301 | 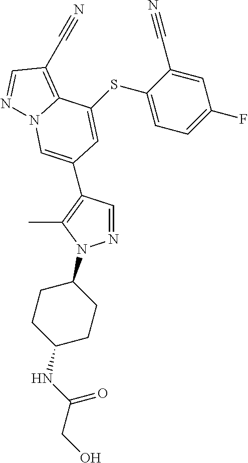 [SMILES: Cc1c(-c2cc(Sc3ccc(F)cc3C#N)c3c(C#N)cnn3c2)cnn1[C@H]1CC[C@H](NC(=O)CO)CC1] |
| 302 | 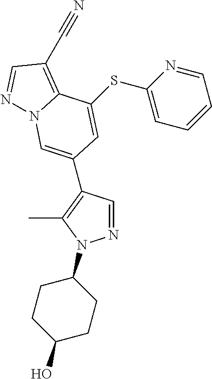 [SMILES: Cc1c(-c2cc(Sc3ccccn3)c3c(C#N)cnn3c2)cnn1[C@H]1CC[C@@H](O)CC1] |
| 303 | 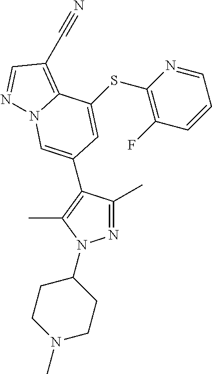 [SMILES: Cc1nn(C2CCN(C)CC2)c(C)c1-c1cc(Sc2ncccc2F)c2c(C#N)cnn2c1] |
| 304 | 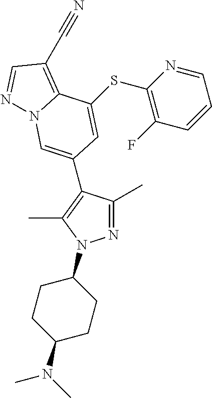 [SMILES: Cc1nn([C@H]2CC[C@@H](N(C)C)CC2)c(C)c1-c1cc(Sc2ncccc2F)c2c(C#N)cnn2c1] |
| 305 | 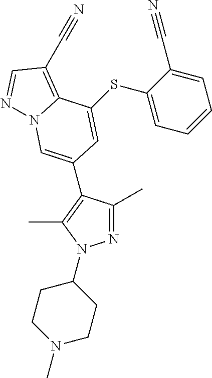 [SMILES: Cc1nn(C2CCN(C)CC2)c(C)c1-c1cc(Sc2ccccc2C#N)c2c(C#N)cnn2c1] |
| 306 | 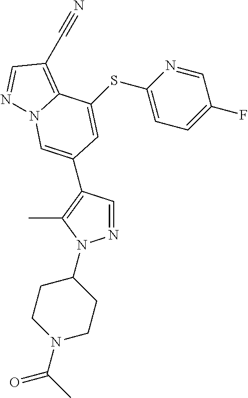 [SMILES: CC(=O)N1CCC(n2ncc(-c3cc(Sc4ccc(F)cn4)c4c(C#N)cnn4c3)c2C)CC1] |
| 307 | 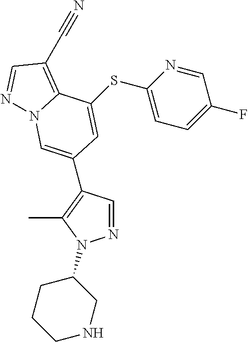 [SMILES: Cc1c(-c2cc(Sc3ccc(F)cn3)c3c(C#N)cnn3c2)cnn1[C@H]1CCCNC1] |
| 308 | 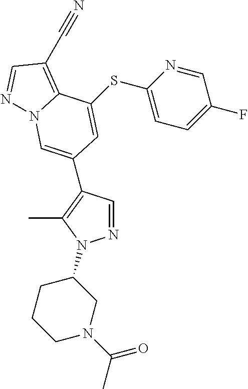 [SMILES: CC(=O)N1CCC[C@H](n2ncc(-c3cc(Sc4ccc(F)cn4)c4c(C#N)cnn4c3)c2C)C1] |
| 309 | 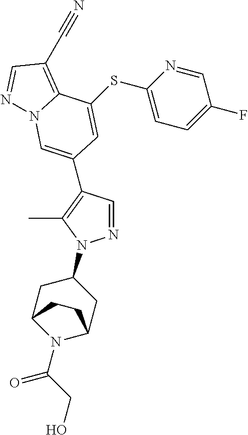 [SMILES: Cc1c(-c2cc(Sc3ccc(F)cn3)c3c(C#N)cnn3c2)cnn1[C@H]1C[C@H]2CC[C@@H](C1)N2C(=O)CO] |
| 310 | 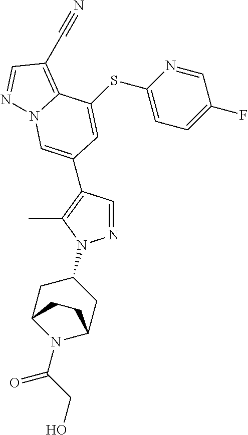 [SMILES: Cc1c(-c2cc(Sc3ccc(F)cn3)c3c(C#N)cnn3c2)cnn1[C@@H]1C[C@H]2CC[C@@H](C1)N2C(=O)CO] |
| 311 | 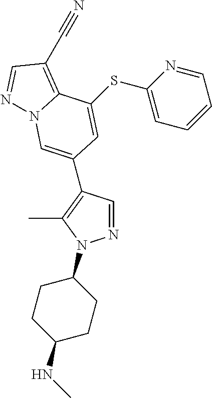 [SMILES: CN[C@H]1CC[C@@H](n2ncc(-c3cc(Sc4ccccn4)c4c(C#N)cnn4c3)c2C)CC1] |
| 312 | 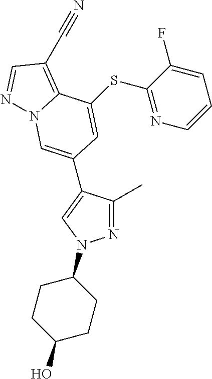 [SMILES: Cc1nn([C@H]2CC[C@@H](O)CC2)cc1-c1cc(Sc2ncccc2F)c2c(C#N)cnn2c1] |
| 313 | 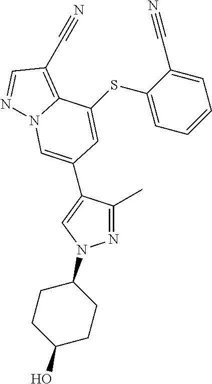 [SMILES: Cc1nn([C@H]2CC[C@@H](O)CC2)cc1-c1cc(Sc2ccccc2C#N)c2c(C#N)cnn2c1] |
| 314 | 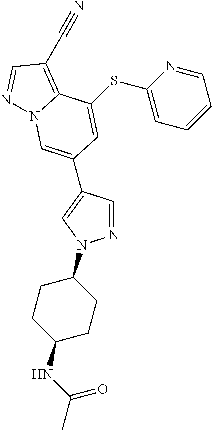 [SMILES: CC(=O)N[C@H]1CC[C@@H](n2cc(-c3cc(Sc4ccccn4)c4c(C#N)cnn4c3)cn2)CC1] |
| 315 | 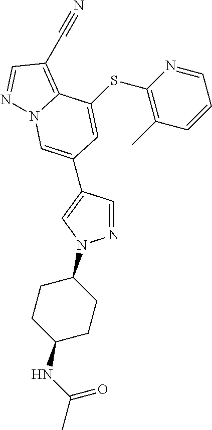 [SMILES: CC(=O)N[C@H]1CC[C@@H](n2cc(-c3cc(Sc4ncccc4C)c4c(C#N)cnn4c3)cn2)CC1] |
| 316 | 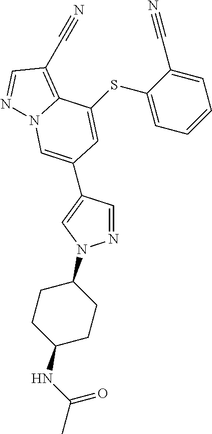 [SMILES: CC(=O)N[C@H]1CC[C@@H](n2cc(-c3cc(Sc4ccccc4C#N)c4c(C#N)cnn4c3)cn2)CC1] |
| 317 | 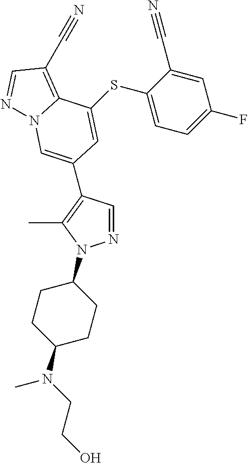 [SMILES: Cc1c(-c2cc(Sc3ccc(F)cc3C#N)c3c(C#N)cnn3c2)cnn1[C@H]1CC[C@@H](N(C)CCO)CC1] |
| 318 | 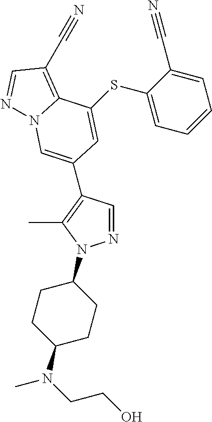 [SMILES: Cc1c(-c2cc(Sc3ccccc3C#N)c3c(C#N)cnn3c2)cnn1[C@H]1CC[C@@H](N(C)CCO)CC1] |
| 319 | 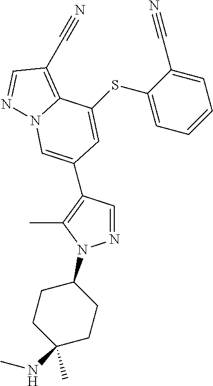 [SMILES: CN[C@]1(C)CC[C@H](n2ncc(-c3cc(Sc4ccccc4C#N)c4c(C#N)cnn4c3)c2C)CC1] |
| 320 | 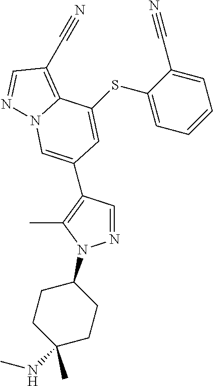 [SMILES: CN[C@]1(C)CC[C@@H](n2ncc(-c3cc(Sc4ccccc4C#N)c4c(C#N)cnn4c3)c2C)CC1] |
| 321 | 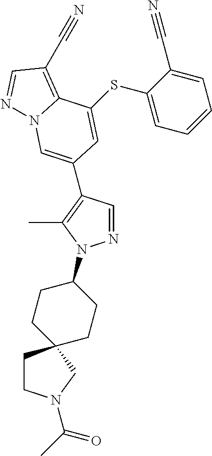 [SMILES: CC(=O)N1CC[C@]2(CC[C@@H](n3ncc(-c4cc(Sc5ccccc5C#N)c5c(C#N)cnn5c4)c3C)CC2)C1] |
| 322 | 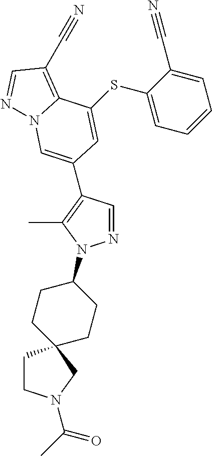 [SMILES: CC(=O)N1CC[C@]2(CC[C@H](n3ncc(-c4cc(Sc5ccccc5C#N)c5c(C#N)cnn5c4)c3C)CC2)C1] |
| 323 | 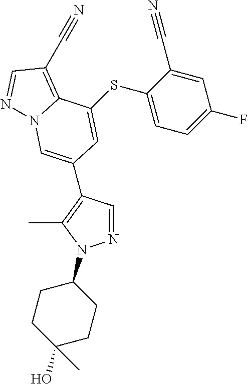 [SMILES: Cc1c(-c2cc(Sc3ccc(F)cc3C#N)c3c(C#N)cnn3c2)cnn1[C@H]1CC[C@](C)(O)CC1] |
| 324 | 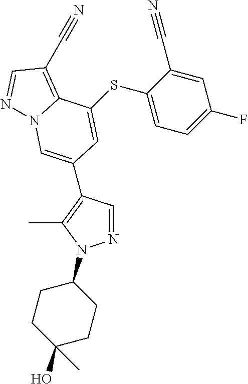 [SMILES: Cc1c(-c2cc(Sc3ccc(F)cc3C#N)c3c(C#N)cnn3c2)cnn1[C@H]1CC[C@@](C)(O)CC1] |
| 325 | 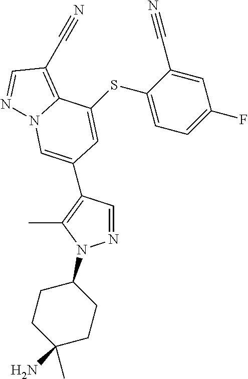 [SMILES: Cc1c(-c2cc(Sc3ccc(F)cc3C#N)c3c(C#N)cnn3c2)cnn1[C@H]1CC[C@@](C)(N)CC1] |
| 326 | 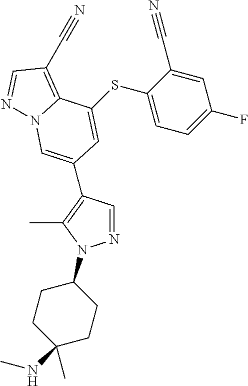 [SMILES: CN[C@]1(C)CC[C@H](n2ncc(-c3cc(Sc4ccc(F)cc4C#N)c4c(C#N)cnn4c3)c2C)CC1] |
| 327 | 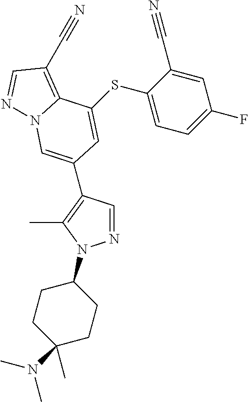 [SMILES: Cc1c(-c2cc(Sc3ccc(F)cc3C#N)c3c(C#N)cnn3c2)cnn1[C@H]1CC[C@@](C)(N(C)C)CC1] |
| 328 | 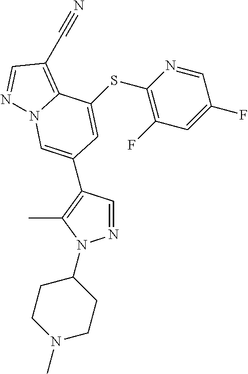 [SMILES: Cc1c(-c2cc(Sc3ncc(F)cc3F)c3c(C#N)cnn3c2)cnn1C1CCN(C)CC1] |
| 329 | 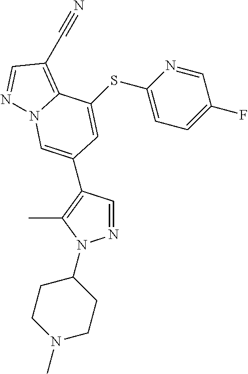 [SMILES: Cc1c(-c2cc(Sc3ccc(F)cn3)c3c(C#N)cnn3c2)cnn1C1CCN(C)CC1] |
| 330 | 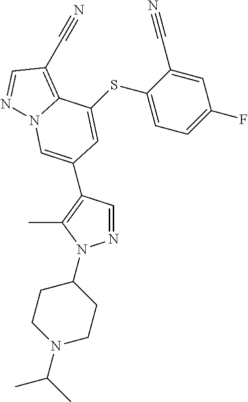 [SMILES: Cc1c(-c2cc(Sc3ccc(F)cc3C#N)c3c(C#N)cnn3c2)cnn1C1CCN(C(C)C)CC1] |
| 331 | 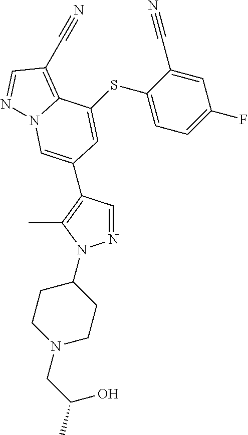 [SMILES: Cc1c(-c2cc(Sc3ccc(F)cc3C#N)c3c(C#N)cnn3c2)cnn1C1CCN(C[C@@H](C)O)CC1] |
| 332 | 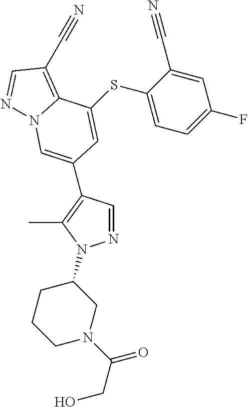 [SMILES: Cc1c(-c2cc(Sc3ccc(F)cc3C#N)c3c(C#N)cnn3c2)cnn1[C@H]1CCCN(C(=O)CO)C1] |
| 333 | 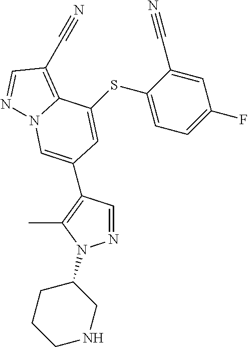 [SMILES: Cc1c(-c2cc(Sc3ccc(F)cc3C#N)c3c(C#N)cnn3c2)cnn1[C@H]1CCCNC1] |
| 334 | 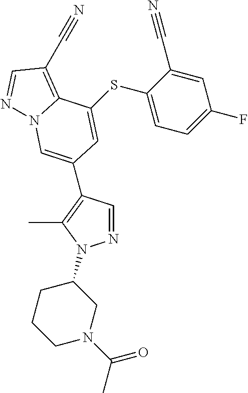 [SMILES: CC(=O)N1CCC[C@H](n2ncc(-c3cc(Sc4ccc(F)cc4C#N)c4c(C#N)cnn4c3)c2C)C1] |
| 335 | 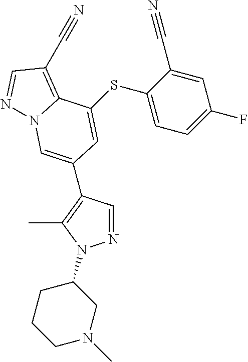 [SMILES: Cc1c(-c2cc(Sc3ccc(F)cc3C#N)c3c(C#N)cnn3c2)cnn1[C@H]1CCCN(C)C1] |
| 336 | 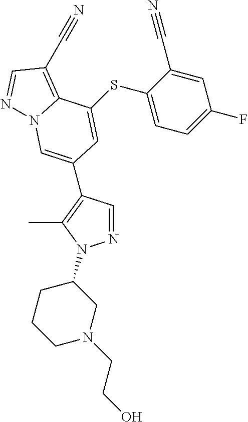 [SMILES: Cc1c(-c2cc(Sc3ccc(F)cc3C#N)c3c(C#N)cnn3c2)cnn1[C@H]1CCCN(CCO)C1] |
| 337 | 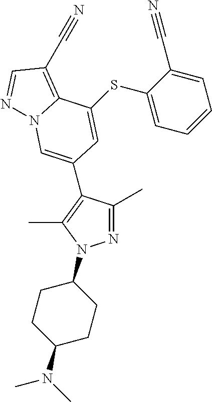 [SMILES: Cc1nn([C@H]2CC[C@@H](N(C)C)CC2)c(C)c1-c1cc(Sc2ccccc2C#N)c2c(C#N)cnn2c1] |
| 338 | 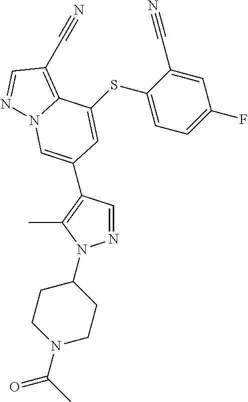 [SMILES: CC(=O)N1CCC(n2ncc(-c3cc(Sc4ccc(F)cc4C#N)c4c(C#N)cnn4c3)c2C)CC1] |
| 339 | 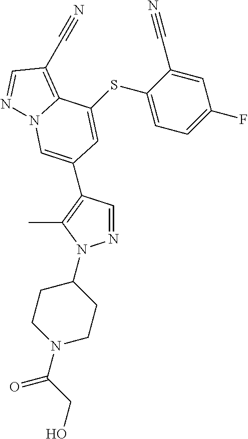 [SMILES: Cc1c(-c2cc(Sc3ccc(F)cc3C#N)c3c(C#N)cnn3c2)cnn1C1CCN(C(=O)CO)CC1] |
| 340 | 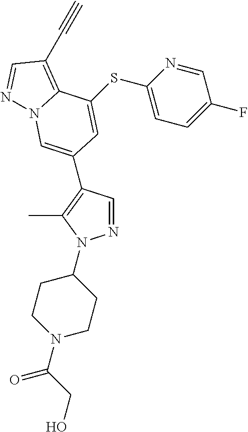 [SMILES: C#Cc1cnn2cc(-c3cnn(C4CCN(C(=O)CO)CC4)c3C)cc(Sc3ccc(F)cn3)c12] |
| 341 | 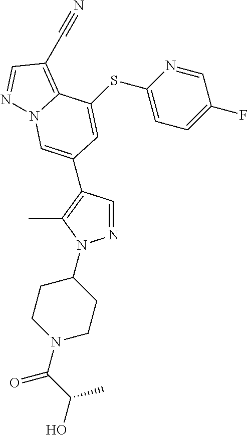 [SMILES: Cc1c(-c2cc(Sc3ccc(F)cn3)c3c(C#N)cnn3c2)cnn1C1CCN(C(=O)[C@H](C)O)CC1] |
| 342 | 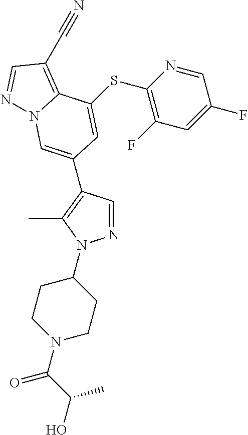 [SMILES: Cc1c(-c2cc(Sc3ncc(F)cc3F)c3c(C#N)cnn3c2)cnn1C1CCN(C(=O)[C@H](C)O)CC1] |
| 343 | 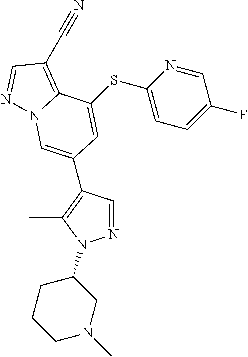 [SMILES: Cc1c(-c2cc(Sc3ccc(F)cn3)c3c(C#N)cnn3c2)cnn1[C@H]1CCCN(C)C1] |
| 344 | 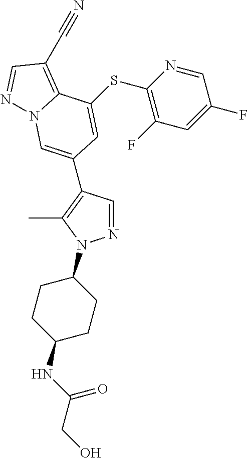 [SMILES: Cc1c(-c2cc(Sc3ncc(F)cc3F)c3c(C#N)cnn3c2)cnn1[C@H]1CC[C@@H](NC(=O)CO)CC1] |
| 345 | 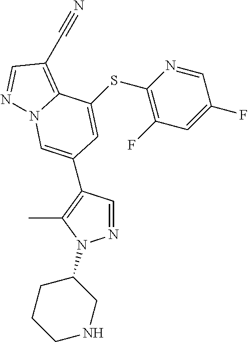 [SMILES: Cc1c(-c2cc(Sc3ncc(F)cc3F)c3c(C#N)cnn3c2)cnn1[C@H]1CCCNC1] |
| 346 | 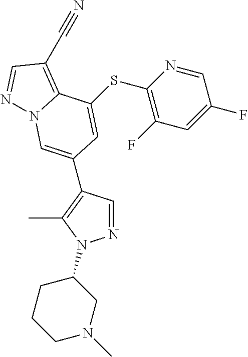 [SMILES: Cc1c(-c2cc(Sc3ncc(F)cc3F)c3c(C#N)cnn3c2)cnn1[C@H]1CCCN(C)C1] |
| 347 | 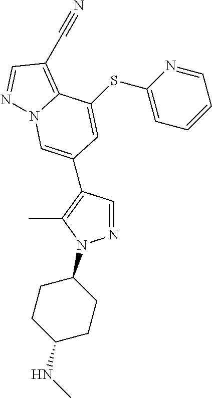 [SMILES: CN[C@H]1CC[C@H](n2ncc(-c3cc(Sc4ccccn4)c4c(C#N)cnn4c3)c2C)CC1] |
| 348 | 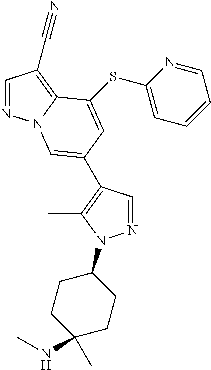 [SMILES: CN[C@]1(C)CC[C@H](n2ncc(-c3cc(Sc4ccccn4)c4c(C#N)cnn4c3)c2C)CC1] |
| 349 | 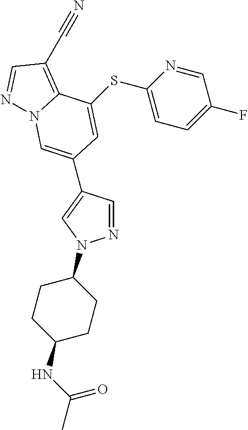 [SMILES: CC(=O)N[C@H]1CC[C@@H](n2cc(-c3cc(Sc4ccc(F)cn4)c4c(C#N)cnn4c3)cn2)CC1] |
| 350 | 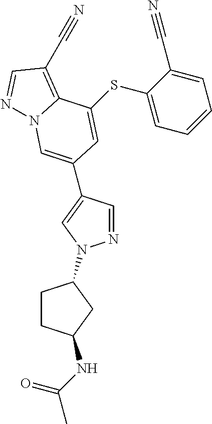 [SMILES: CC(=O)N[C@H]1CC[C@H](n2cc(-c3cc(Sc4ccccc4C#N)c4c(C#N)cnn4c3)cn2)C1] |
| 351 | 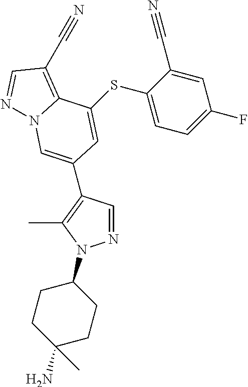 [SMILES: Cc1c(-c2cc(Sc3ccc(F)cc3C#N)c3c(C#N)cnn3c2)cnn1[C@H]1CC[C@](C)(N)CC1] |
| 352 | 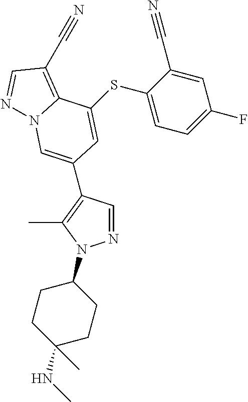 [SMILES: CN[C@]1(C)CC[C@@H](n2ncc(-c3cc(Sc4ccc(F)cc4C#N)c4c(C#N)cnn4c3)c2C)CC1] |
| 353 | 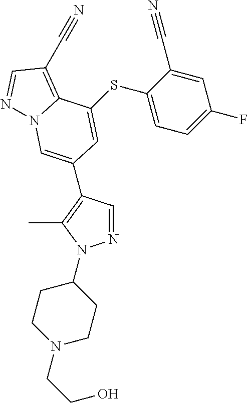 [SMILES: Cc1c(-c2cc(Sc3ccc(F)cc3C#N)c3c(C#N)cnn3c2)cnn1C1CCN(CCO)CC1] |
| 354 | 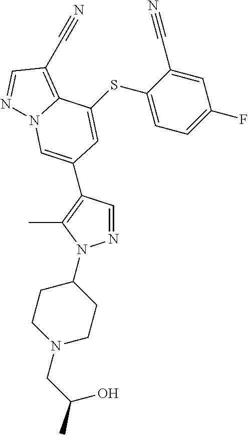 [SMILES: Cc1c(-c2cc(Sc3ccc(F)cc3C#N)c3c(C#N)cnn3c2)cnn1C1CCN(C[C@H](C)O)CC1] |
| 355 | 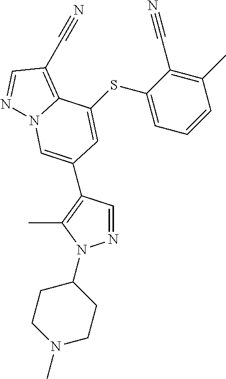 [SMILES: Cc1cccc(Sc2cc(-c3cnn(C4CCN(C)CC4)c3C)cn3ncc(C#N)c23)c1C#N] |
| 356 | 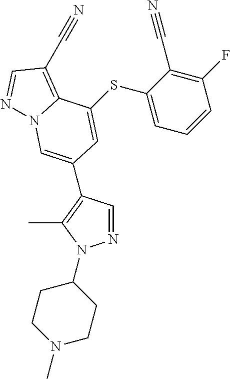 [SMILES: Cc1c(-c2cc(Sc3cccc(F)c3C#N)c3c(C#N)cnn3c2)cnn1C1CCN(C)CC1] |
| 357 | 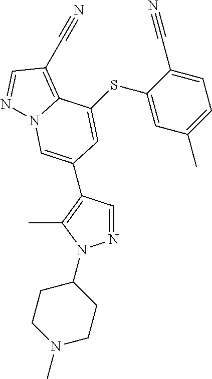 [SMILES: Cc1ccc(C#N)c(Sc2cc(-c3cnn(C4CCN(C)CC4)c3C)cn3ncc(C#N)c23)c1] |
| 358 | 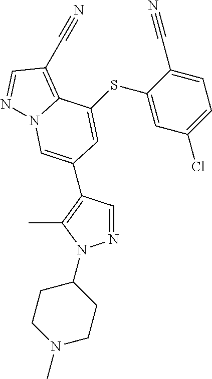 [SMILES: Cc1c(-c2cc(Sc3cc(Cl)ccc3C#N)c3c(C#N)cnn3c2)cnn1C1CCN(C)CC1] |
| 359 | 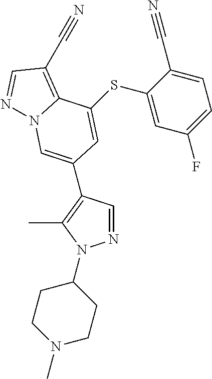 [SMILES: Cc1c(-c2cc(Sc3cc(F)ccc3C#N)c3c(C#N)cnn3c2)cnn1C1CCN(C)CC1] |
| 360 | 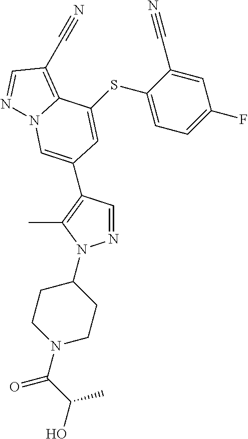 [SMILES: Cc1c(-c2cc(Sc3ccc(F)cc3C#N)c3c(C#N)cnn3c2)cnn1C1CCN(C(=O)[C@H](C)O)CC1] |
| 361 | 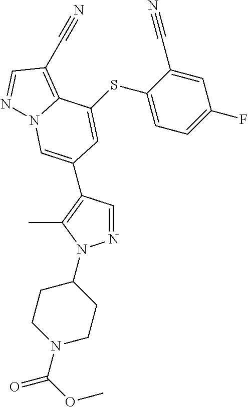 [SMILES: COC(=O)N1CCC(n2ncc(-c3cc(Sc4ccc(F)cc4C#N)c4c(C#N)cnn4c3)c2C)CC1] |
| 362 | 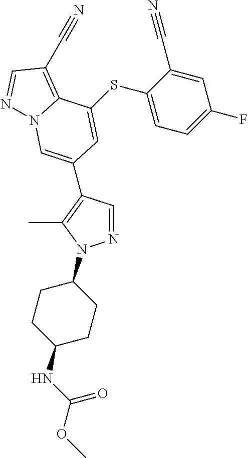 [SMILES: COC(=O)N[C@H]1CC[C@@H](n2ncc(-c3cc(Sc4ccc(F)cc4C#N)c4c(C#N)cnn4c3)c2C)CC1] |
| 363 | 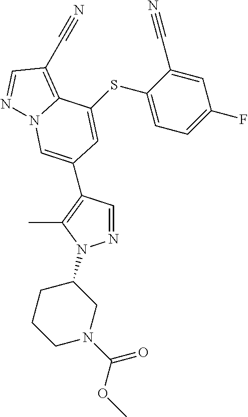 [SMILES: COC(=O)N1CCC[C@H](n2ncc(-c3cc(Sc4ccc(F)cc4C#N)c4c(C#N)cnn4c3)c2C)C1] |
| 364 | 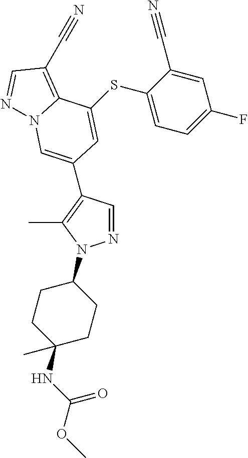 [SMILES: COC(=O)N[C@]1(C)CC[C@H](n2ncc(-c3cc(Sc4ccc(F)cc4C#N)c4c(C#N)cnn4c3)c2C)CC1] |
| 365 | 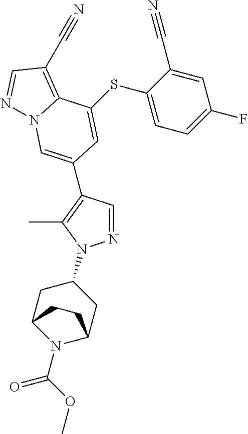 [SMILES: COC(=O)N1[C@@H]2CC[C@H]1C[C@H](n1ncc(-c3cc(Sc4ccc(F)cc4C#N)c4c(C#N)cnn4c3)c1C)C2] |
| 366 | 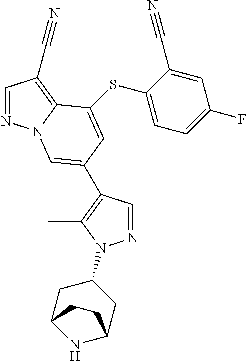 [SMILES: Cc1c(-c2cc(Sc3ccc(F)cc3C#N)c3c(C#N)cnn3c2)cnn1[C@@H]1C[C@H]2CC[C@@H](C1)N2] |
| 367 | 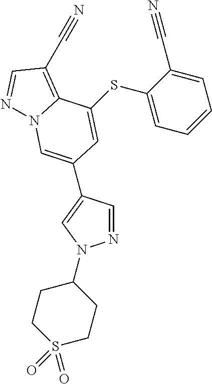 [SMILES: N#Cc1ccccc1Sc1cc(-c2cnn(C3CCS(=O)(=O)CC3)c2)cn2ncc(C#N)c12] |
| 368 | 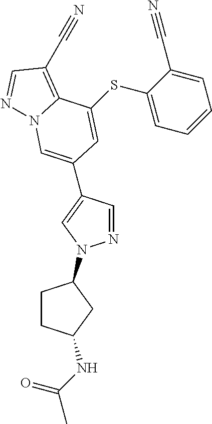 [SMILES: CC(=O)N[C@@H]1CC[C@@H](n2cc(-c3cc(Sc4ccccc4C#N)c4c(C#N)cnn4c3)cn2)C1] |
| 369 | 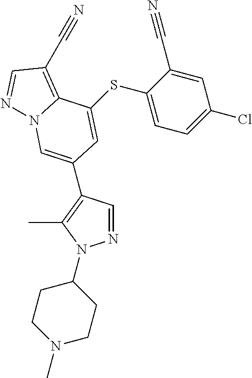 [SMILES: Cc1c(-c2cc(Sc3ccc(Cl)cc3C#N)c3c(C#N)cnn3c2)cnn1C1CCN(C)CC1] |
| 370 | 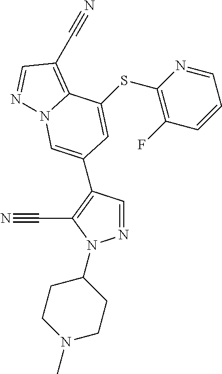 [SMILES: CN1CCC(n2ncc(-c3cc(Sc4ncccc4F)c4c(C#N)cnn4c3)c2C#N)CC1] |
| 371 | 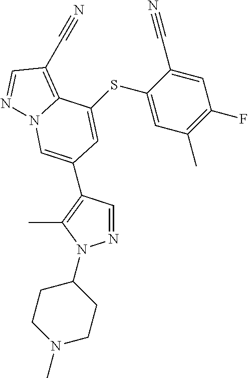 [SMILES: Cc1cc(Sc2cc(-c3cnn(C4CCN(C)CC4)c3C)cn3ncc(C#N)c23)c(C#N)cc1F] |
| 372 | 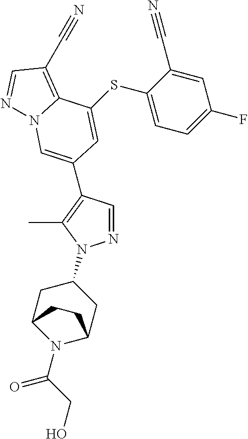 [SMILES: Cc1c(-c2cc(Sc3ccc(F)cc3C#N)c3c(C#N)cnn3c2)cnn1[C@@H]1C[C@H]2CC[C@@H](C1)N2C(=O)CO] |
| 373 | 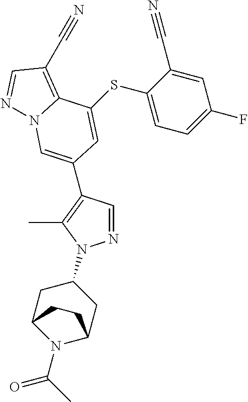 [SMILES: CC(=O)N1[C@@H]2CC[C@H]1C[C@H](n1ncc(-c3cc(Sc4ccc(F)cc4C#N)c4c(C#N)cnn4c3)c1C)C2] |
| 374 | 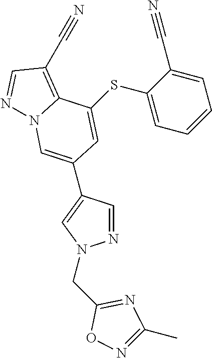 [SMILES: Cc1noc(Cn2cc(-c3cc(Sc4ccccc4C#N)c4c(C#N)cnn4c3)cn2)n1] |
| 375 | 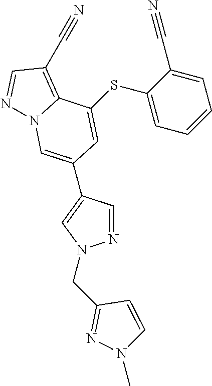 [SMILES: Cn1ccc(Cn2cc(-c3cc(Sc4ccccc4C#N)c4c(C#N)cnn4c3)cn2)n1] |
| 376 | 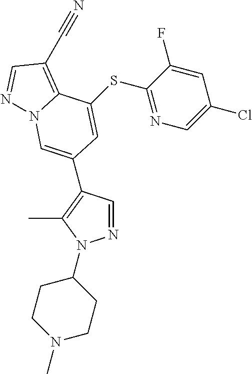 [SMILES: Cc1c(-c2cc(Sc3ncc(Cl)cc3F)c3c(C#N)cnn3c2)cnn1C1CCN(C)CC1] |
| 377 | 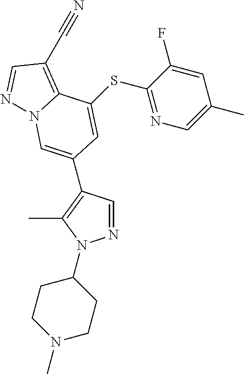 [SMILES: Cc1cnc(Sc2cc(-c3cnn(C4CCN(C)CC4)c3C)cn3ncc(C#N)c23)c(F)c1] |
| 378 | 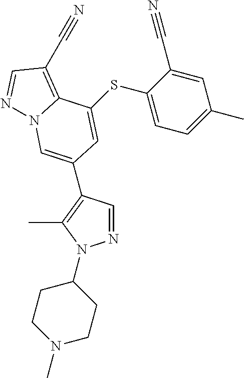 [SMILES: Cc1ccc(Sc2cc(-c3cnn(C4CCN(C)CC4)c3C)cn3ncc(C#N)c23)c(C#N)c1] |
| 379 | 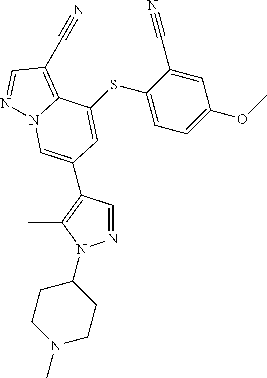 [SMILES: COc1ccc(Sc2cc(-c3cnn(C4CCN(C)CC4)c3C)cn3ncc(C#N)c23)c(C#N)c1] |
| 380 | 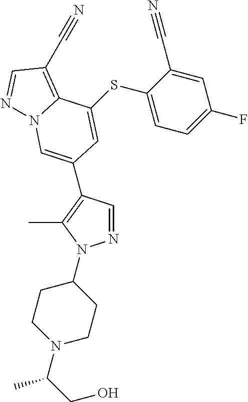 [SMILES: Cc1c(-c2cc(Sc3ccc(F)cc3C#N)c3c(C#N)cnn3c2)cnn1C1CCN([C@@H](C)CO)CC1] |
| 381 | 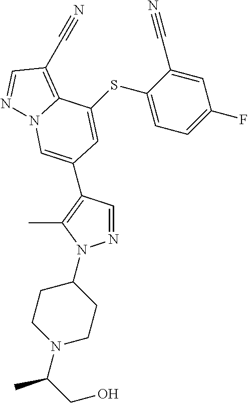 [SMILES: Cc1c(-c2cc(Sc3ccc(F)cc3C#N)c3c(C#N)cnn3c2)cnn1C1CCN([C@H](C)CO)CC1] |
| 382 | 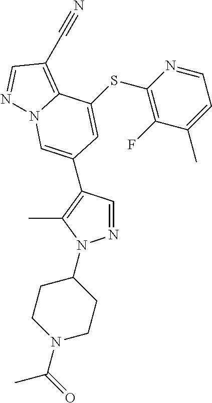 [SMILES: CC(=O)N1CCC(n2ncc(-c3cc(Sc4nccc(C)c4F)c4c(C#N)cnn4c3)c2C)CC1] |
| 383 | 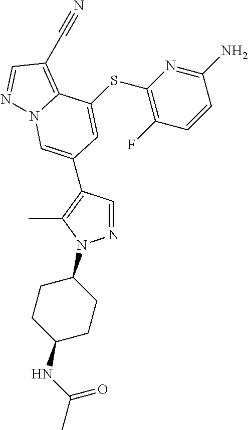 [SMILES: CC(=O)N[C@H]1CC[C@@H](n2ncc(-c3cc(Sc4nc(N)ccc4F)c4c(C#N)cnn4c3)c2C)CC1] |
| 384 | 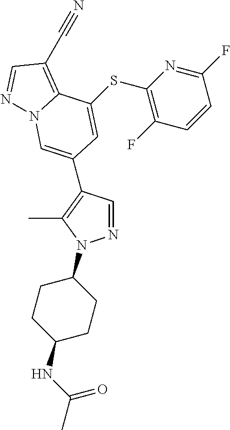 [SMILES: CC(=O)N[C@H]1CC[C@@H](n2ncc(-c3cc(Sc4nc(F)ccc4F)c4c(C#N)cnn4c3)c2C)CC1] |
| 385 | 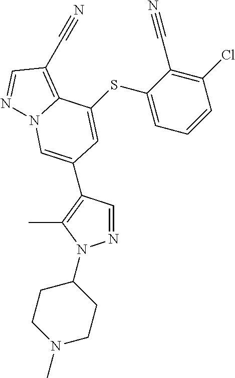 [SMILES: Cc1c(-c2cc(Sc3cccc(Cl)c3C#N)c3c(C#N)cnn3c2)cnn1C1CCN(C)CC1] |
| 386 | 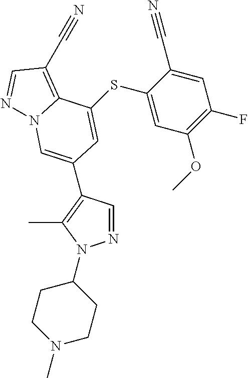 [SMILES: COc1cc(Sc2cc(-c3cnn(C4CCN(C)CC4)c3C)cn3ncc(C#N)c23)c(C#N)cc1F] |
| 387 | 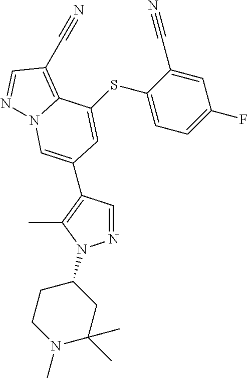 [SMILES: Cc1c(-c2cc(Sc3ccc(F)cc3C#N)c3c(C#N)cnn3c2)cnn1[C@H]1CCN(C)C(C)(C)C1] |
| 388 | 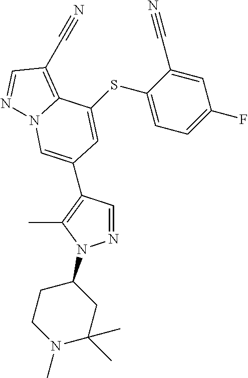 [SMILES: Cc1c(-c2cc(Sc3ccc(F)cc3C#N)c3c(C#N)cnn3c2)cnn1[C@@H]1CCN(C)C(C)(C)C1] |
| 389 | 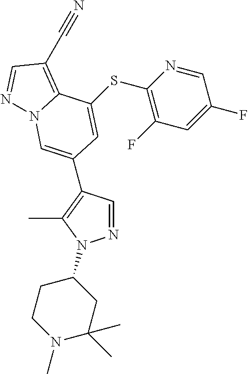 [SMILES: Cc1c(-c2cc(Sc3ncc(F)cc3F)c3c(C#N)cnn3c2)cnn1[C@H]1CCN(C)C(C)(C)C1] |
| 390 | 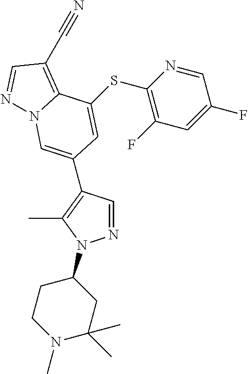 [SMILES: Cc1c(-c2cc(Sc3ncc(F)cc3F)c3c(C#N)cnn3c2)cnn1[C@@H]1CCN(C)C(C)(C)C1] |
| 391 | 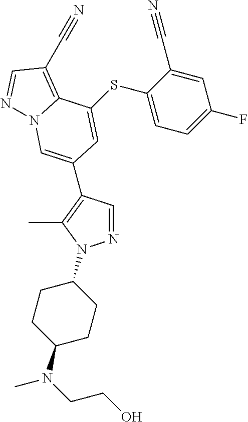 [SMILES: Cc1c(-c2cc(Sc3ccc(F)cc3C#N)c3c(C#N)cnn3c2)cnn1[C@H]1CC[C@H](N(C)CCO)CC1] |
| 392 | 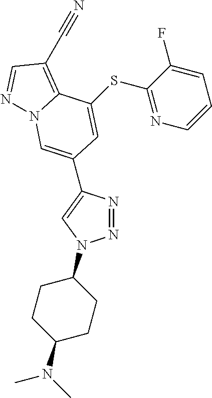 [SMILES: CN(C)[C@H]1CC[C@@H](n2cc(-c3cc(Sc4ncccc4F)c4c(C#N)cnn4c3)nn2)CC1] |
| 393 | 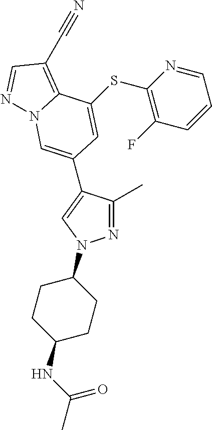 [SMILES: CC(=O)N[C@H]1CC[C@@H](n2cc(-c3cc(Sc4ncccc4F)c4c(C#N)cnn4c3)c(C)n2)CC1] |
| 394 | 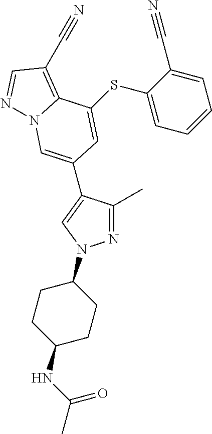 [SMILES: CC(=O)N[C@H]1CC[C@@H](n2cc(-c3cc(Sc4ccccc4C#N)c4c(C#N)cnn4c3)c(C)n2)CC1] |
| 395 | 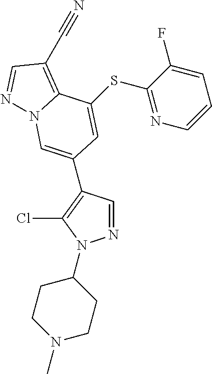 [SMILES: CN1CCC(n2ncc(-c3cc(Sc4ncccc4F)c4c(C#N)cnn4c3)c2Cl)CC1] |
| 396 | 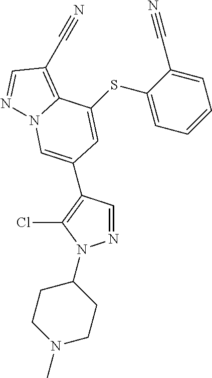 [SMILES: CN1CCC(n2ncc(-c3cc(Sc4ccccc4C#N)c4c(C#N)cnn4c3)c2Cl)CC1] |
| 397 | 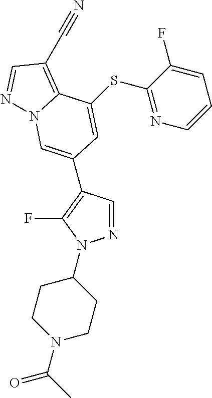 [SMILES: CC(=O)N1CCC(n2ncc(-c3cc(Sc4ncccc4F)c4c(C#N)cnn4c3)c2F)CC1] |
| 398 | 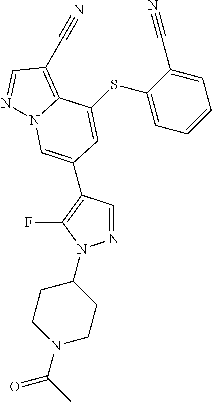 [SMILES: CC(=O)N1CCC(n2ncc(-c3cc(Sc4ccccc4C#N)c4c(C#N)cnn4c3)c2F)CC1] |
| 399 | 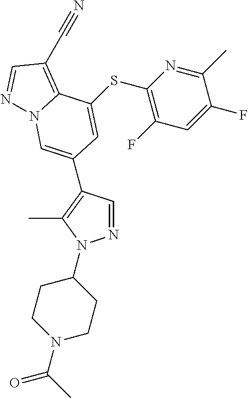 [SMILES: CC(=O)N1CCC(n2ncc(-c3cc(Sc4nc(C)c(F)cc4F)c4c(C#N)cnn4c3)c2C)CC1] |
| 400 | 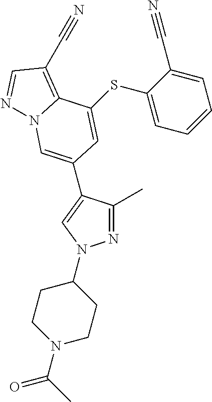 [SMILES: CC(=O)N1CCC(n2cc(-c3cc(Sc4ccccc4C#N)c4c(C#N)cnn4c3)c(C)n2)CC1] |
| 401 | 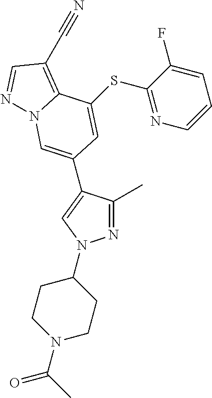 [SMILES: CC(=O)N1CCC(n2cc(-c3cc(Sc4ncccc4F)c4c(C#N)cnn4c3)c(C)n2)CC1] |
| 402 | 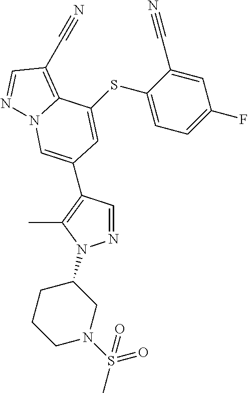 [SMILES: Cc1c(-c2cc(Sc3ccc(F)cc3C#N)c3c(C#N)cnn3c2)cnn1[C@H]1CCCN(S(C)(=O)=O)C1] |
| 403 | 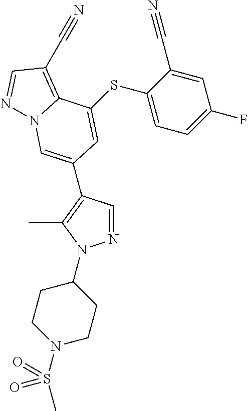 [SMILES: Cc1c(-c2cc(Sc3ccc(F)cc3C#N)c3c(C#N)cnn3c2)cnn1C1CCN(S(C)(=O)=O)CC1] |
| 404 | 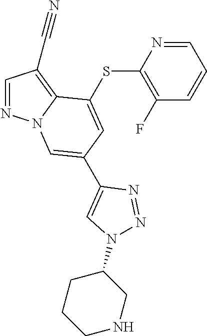 [SMILES: N#Cc1cnn2cc(-c3cn([C@H]4CCCNC4)nn3)cc(Sc3ncccc3F)c12] |
| 405 | 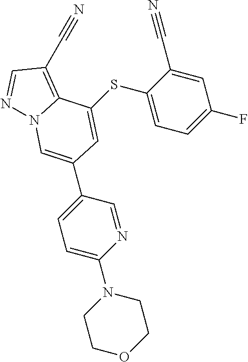 [SMILES: N#Cc1cc(F)ccc1Sc1cc(-c2ccc(N3CCOCC3)nc2)cn2ncc(C#N)c12] |
| 406 | 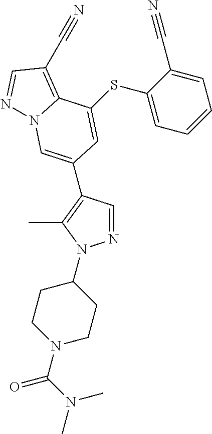 [SMILES: Cc1c(-c2cc(Sc3ccccc3C#N)c3c(C#N)cnn3c2)cnn1C1CCN(C(=O)N(C)C)CC1] |
| 407 | 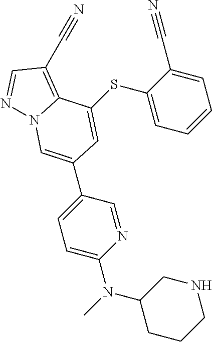 [SMILES: CN(c1ccc(-c2cc(Sc3ccccc3C#N)c3c(C#N)cnn3c2)cn1)C1CCCNC1] |
| 408 | 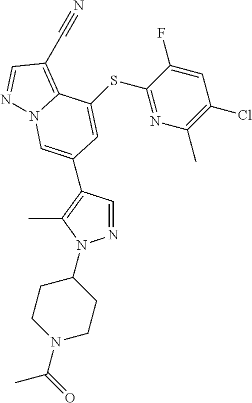 [SMILES: CC(=O)N1CCC(n2ncc(-c3cc(Sc4nc(C)c(Cl)cc4F)c4c(C#N)cnn4c3)c2C)CC1] |
| 409 | 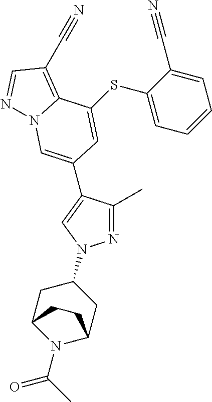 [SMILES: CC(=O)N1[C@@H]2CC[C@H]1C[C@H](n1cc(-c3cc(Sc4ccccc4C#N)c4c(C#N)cnn4c3)c(C)n1)C2] |
| 410 | 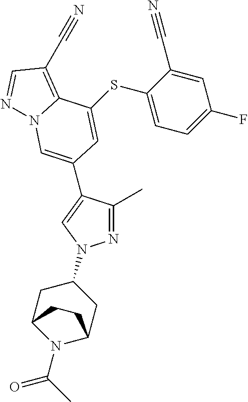 [SMILES: CC(=O)N1[C@@H]2CC[C@H]1C[C@H](n1cc(-c3cc(Sc4ccc(F)cc4C#N)c4c(C#N)cnn4c3)c(C)n1)C2] |
| 411 | 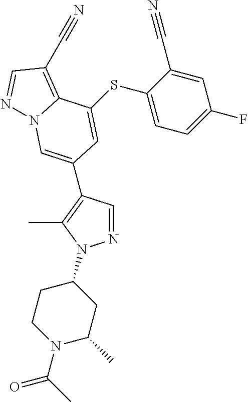 [SMILES: CC(=O)N1CC[C@H](n2ncc(-c3cc(Sc4ccc(F)cc4C#N)c4c(C#N)cnn4c3)c2C)C[C@@H]1C] |
| 412 | 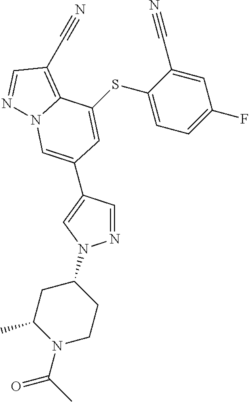 [SMILES: CC(=O)N1CC[C@@H](n2cc(-c3cc(Sc4ccc(F)cc4C#N)c4c(C#N)cnn4c3)cn2)C[C@H]1C] |
| 413 | 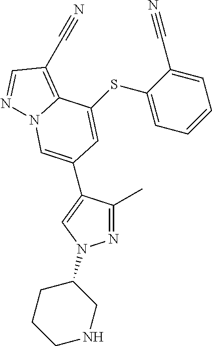 [SMILES: Cc1nn([C@H]2CCCNC2)cc1-c1cc(Sc2ccccc2C#N)c2c(C#N)cnn2c1] |
| 414 | 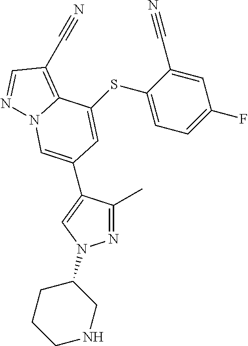 [SMILES: Cc1nn([C@H]2CCCNC2)cc1-c1cc(Sc2ccc(F)cc2C#N)c2c(C#N)cnn2c1] |
| 415 | 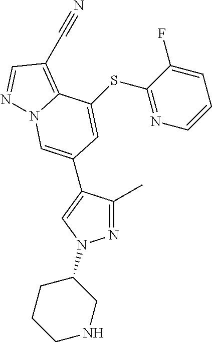 [SMILES: Cc1nn([C@H]2CCCNC2)cc1-c1cc(Sc2ncccc2F)c2c(C#N)cnn2c1] |
| 416 | 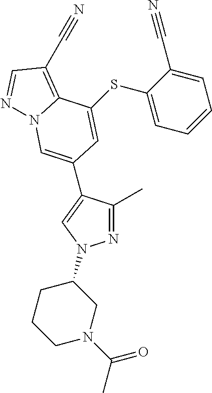 [SMILES: CC(=O)N1CCC[C@H](n2cc(-c3cc(Sc4ccccc4C#N)c4c(C#N)cnn4c3)c(C)n2)C1] |
| 417 | 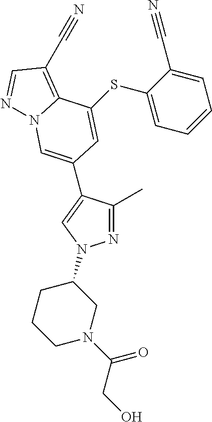 [SMILES: Cc1nn([C@H]2CCCN(C(=O)CO)C2)cc1-c1cc(Sc2ccccc2C#N)c2c(C#N)cnn2c1] |
| 418 | 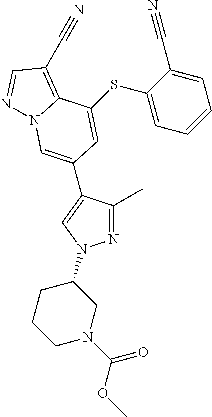 [SMILES: COC(=O)N1CCC[C@H](n2cc(-c3cc(Sc4ccccc4C#N)c4c(C#N)cnn4c3)c(C)n2)C1] |
| 419 | 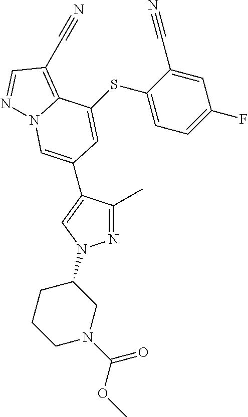 [SMILES: COC(=O)N1CCC[C@H](n2cc(-c3cc(Sc4ccc(F)cc4C#N)c4c(C#N)cnn4c3)c(C)n2)C1] |
| 420 | 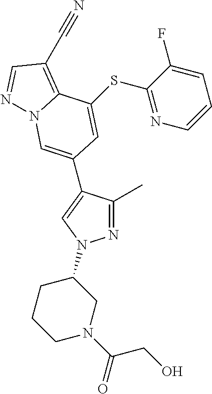 [SMILES: Cc1nn([C@H]2CCCN(C(=O)CO)C2)cc1-c1cc(Sc2ncccc2F)c2c(C#N)cnn2c1] |
| 421 | 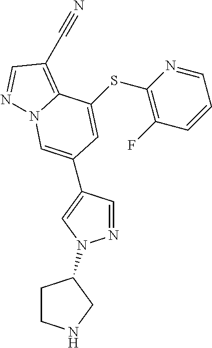 [SMILES: N#Cc1cnn2cc(-c3cnn([C@H]4CCNC4)c3)cc(Sc3ncccc3F)c12] |
| 422 | 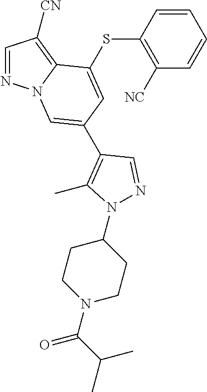 [SMILES: Cc1c(-c2cc(Sc3ccccc3C#N)c3c(C#N)cnn3c2)cnn1C1CCN(C(=O)C(C)C)CC1] |
| 423 | 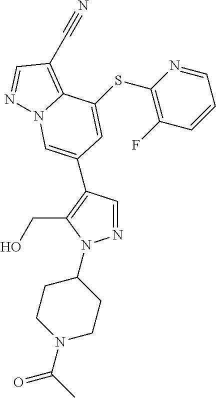 [SMILES: CC(=O)N1CCC(n2ncc(-c3cc(Sc4ncccc4F)c4c(C#N)cnn4c3)c2CO)CC1] |
| 424 | 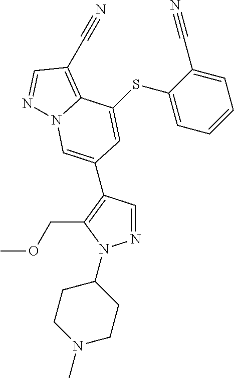 [SMILES: COCc1c(-c2cc(Sc3ccccc3C#N)c3c(C#N)cnn3c2)cnn1C1CCN(C)CC1] |
| 425 | 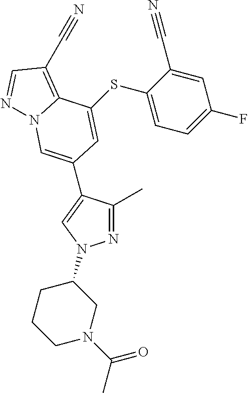 [SMILES: CC(=O)N1CCC[C@H](n2cc(-c3cc(Sc4ccc(F)cc4C#N)c4c(C#N)cnn4c3)c(C)n2)C1] |
| 426 | 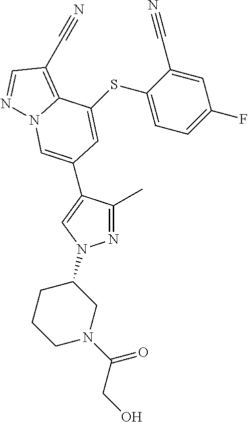 [SMILES: Cc1nn([C@H]2CCCN(C(=O)CO)C2)cc1-c1cc(Sc2ccc(F)cc2C#N)c2c(C#N)cnn2c1] |
| 427 | 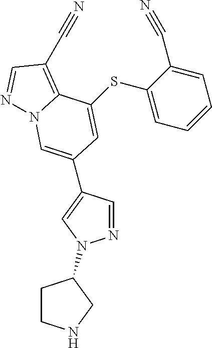 [SMILES: N#Cc1ccccc1Sc1cc(-c2cnn([C@H]3CCNC3)c2)cn2ncc(C#N)c12] |
| 428 | 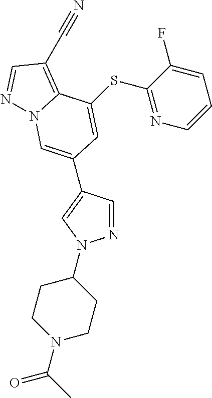 [SMILES: CC(=O)N1CCC(n2cc(-c3cc(Sc4ncccc4F)c4c(C#N)cnn4c3)cn2)CC1] |
| 429 | 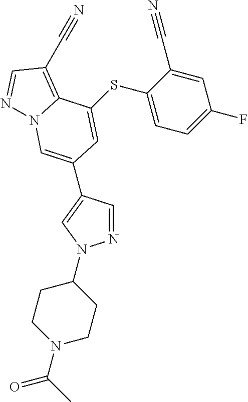 [SMILES: CC(=O)N1CCC(n2cc(-c3cc(Sc4ccc(F)cc4C#N)c4c(C#N)cnn4c3)cn2)CC1] |
| 430 | 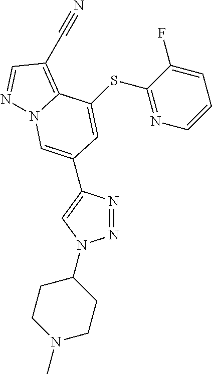 [SMILES: CN1CCC(n2cc(-c3cc(Sc4ncccc4F)c4c(C#N)cnn4c3)nn2)CC1] |
| 431 | 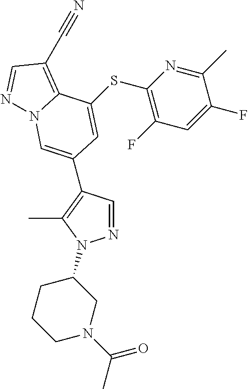 [SMILES: CC(=O)N1CCC[C@H](n2ncc(-c3cc(Sc4nc(C)c(F)cc4F)c4c(C#N)cnn4c3)c2C)C1] |
| 432 | 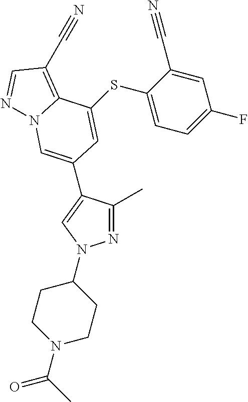 [SMILES: CC(=O)N1CCC(n2cc(-c3cc(Sc4ccc(F)cc4C#N)c4c(C#N)cnn4c3)c(C)n2)CC1] |
| 433 | 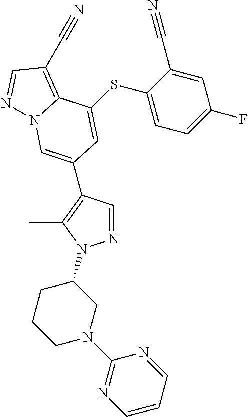 [SMILES: Cc1c(-c2cc(Sc3ccc(F)cc3C#N)c3c(C#N)cnn3c2)cnn1[C@H]1CCCN(c2ncccn2)C1] |
| 434 | 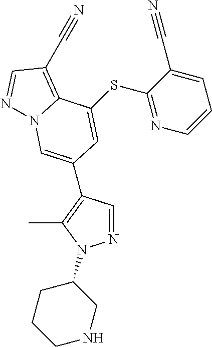 [SMILES: Cc1c(-c2cc(Sc3ncccc3C#N)c3c(C#N)cnn3c2)cnn1[C@H]1CCCNC1] |
| 435 | 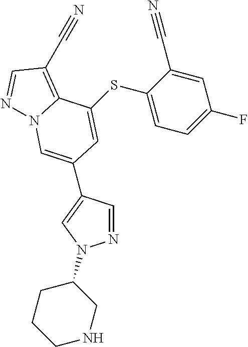 [SMILES: N#Cc1cc(F)ccc1Sc1cc(-c2cnn([C@H]3CCCNC3)c2)cn2ncc(C#N)c12] |
| 436 | 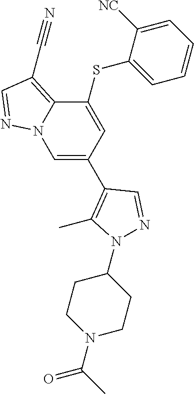 [SMILES: CC(=O)N1CCC(n2ncc(-c3cc(Sc4ccccc4C#N)c4c(C#N)cnn4c3)c2C)CC1] |
| 437 | 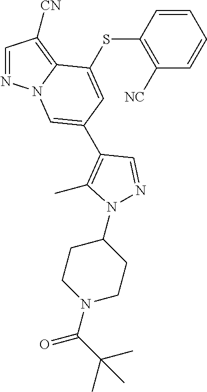 [SMILES: Cc1c(-c2cc(Sc3ccccc3C#N)c3c(C#N)cnn3c2)cnn1C1CCN(C(=O)C(C)(C)C)CC1] |
| 438 | 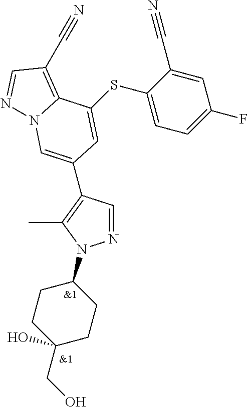 [SMILES: Cc1c(-c2cc(Sc3ccc(F)cc3C#N)c3c(C#N)cnn3c2)cnn1[C@H]1CC[C@@](O)(CO)CC1] |
| 439 | 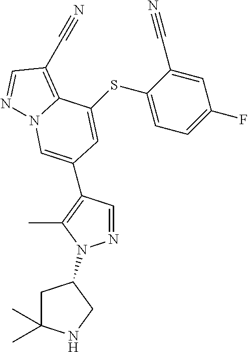 [SMILES: Cc1c(-c2cc(Sc3ccc(F)cc3C#N)c3c(C#N)cnn3c2)cnn1[C@@H]1CNC(C)(C)C1] |
| 440 | 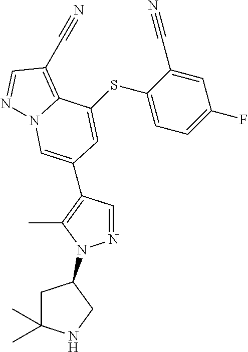 [SMILES: Cc1c(-c2cc(Sc3ccc(F)cc3C#N)c3c(C#N)cnn3c2)cnn1[C@H]1CNC(C)(C)C1] |
| 441 | 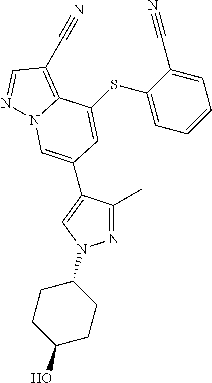 [SMILES: Cc1nn([C@H]2CC[C@H](O)CC2)cc1-c1cc(Sc2ccccc2C#N)c2c(C#N)cnn2c1] |
| 442 | 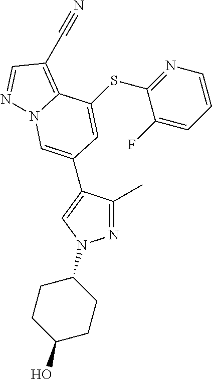 [SMILES: Cc1nn([C@H]2CC[C@H](O)CC2)cc1-c1cc(Sc2ncccc2F)c2c(C#N)cnn2c1] |
| 443 | 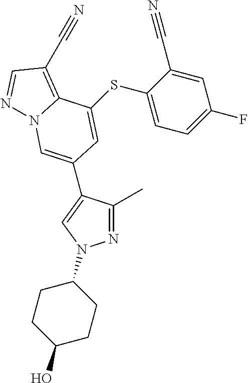 [SMILES: Cc1nn([C@H]2CC[C@H](O)CC2)cc1-c1cc(Sc2ccc(F)cc2C#N)c2c(C#N)cnn2c1] |
| 444 | 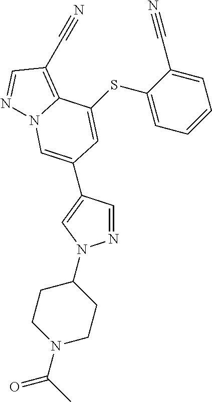 [SMILES: CC(=O)N1CCC(n2cc(-c3cc(Sc4ccccc4C#N)c4c(C#N)cnn4c3)cn2)CC1] |
| 445 | 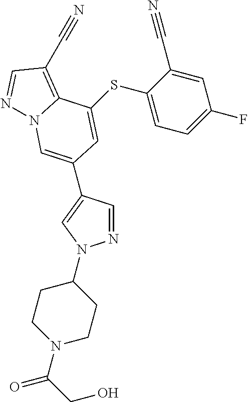 [SMILES: N#Cc1cc(F)ccc1Sc1cc(-c2cnn(C3CCN(C(=O)CO)CC3)c2)cn2ncc(C#N)c12] |
| 446 | 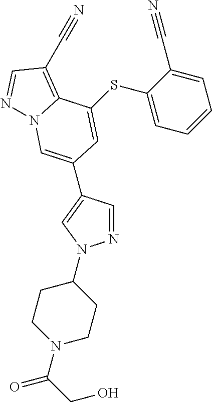 [SMILES: N#Cc1ccccc1Sc1cc(-c2cnn(C3CCN(C(=O)CO)CC3)c2)cn2ncc(C#N)c12] |
| 447 | 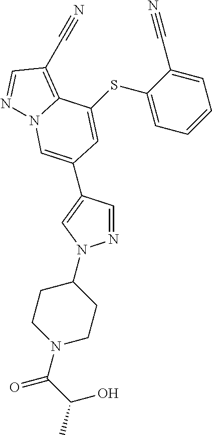 [SMILES: C[C@@H](O)C(=O)N1CCC(n2cc(-c3cc(Sc4ccccc4C#N)c4c(C#N)cnn4c3)cn2)CC1] |
| 448 | 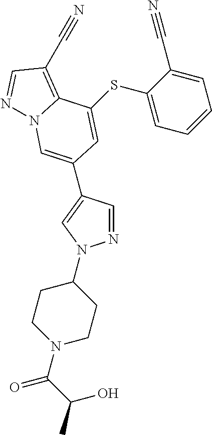 [SMILES: C[C@H](O)C(=O)N1CCC(n2cc(-c3cc(Sc4ccccc4C#N)c4c(C#N)cnn4c3)cn2)CC1] |
| 449 | 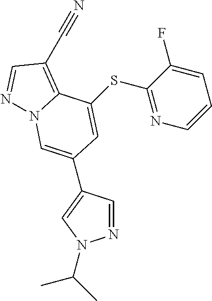 [SMILES: CC(C)n1cc(-c2cc(Sc3ncccc3F)c3c(C#N)cnn3c2)cn1] |
| 450 | 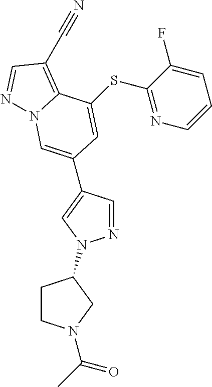 [SMILES: CC(=O)N1CC[C@H](n2cc(-c3cc(Sc4ncccc4F)c4c(C#N)cnn4c3)cn2)C1] |
| 451 | 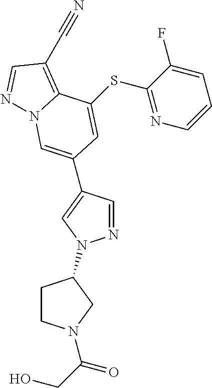 [SMILES: N#Cc1cnn2cc(-c3cnn([C@H]4CCN(C(=O)CO)C4)c3)cc(Sc3ncccc3F)c12] |
| 452 | 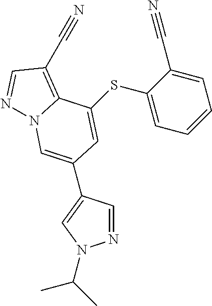 [SMILES: CC(C)n1cc(-c2cc(Sc3ccccc3C#N)c3c(C#N)cnn3c2)cn1] |
| 453 | 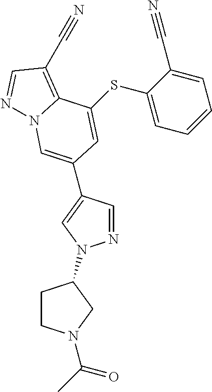 [SMILES: CC(=O)N1CC[C@H](n2cc(-c3cc(Sc4ccccc4C#N)c4c(C#N)cnn4c3)cn2)C1] |
| 454 | 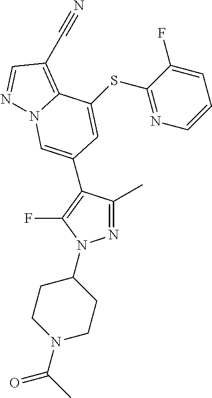 [SMILES: CC(=O)N1CCC(n2nc(C)c(-c3cc(Sc4ncccc4F)c4c(C#N)cnn4c3)c2F)CC1] |
| 455 | 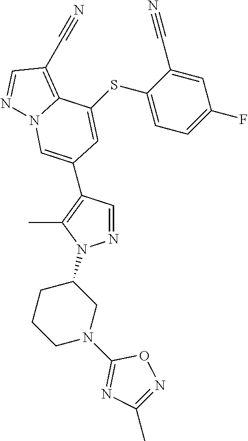 [SMILES: Cc1noc(N2CCC[C@H](n3ncc(-c4cc(Sc5ccc(F)cc5C#N)c5c(C#N)cnn5c4)c3C)C2)n1] |
| 456 | 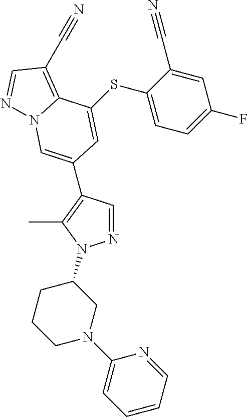 [SMILES: Cc1c(-c2cc(Sc3ccc(F)cc3C#N)c3c(C#N)cnn3c2)cnn1[C@H]1CCCN(c2ccccn2)C1] |
| 457 | 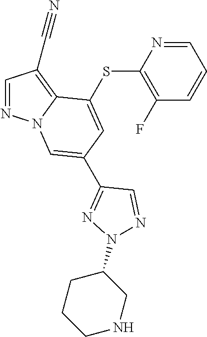 [SMILES: N#Cc1cnn2cc(-c3cnn([C@H]4CCCNC4)n3)cc(Sc3ncccc3F)c12] |
| 458 | 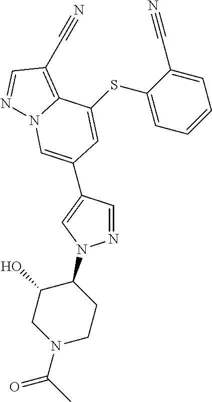 [SMILES: CC(=O)N1CC[C@H](n2cc(-c3cc(Sc4ccccc4C#N)c4c(C#N)cnn4c3)cn2)[C@@H](O)C1] |
| 459 | 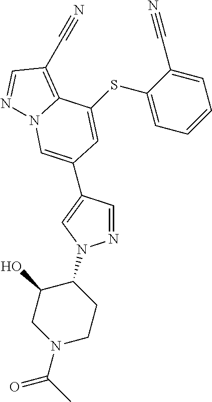 [SMILES: CC(=O)N1CC[C@@H](n2cc(-c3cc(Sc4ccccc4C#N)c4c(C#N)cnn4c3)cn2)[C@H](O)C1] |
| 460 | 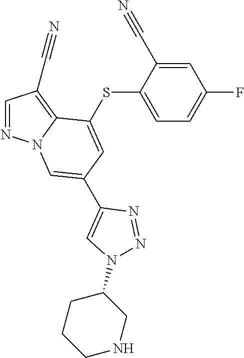 [SMILES: N#Cc1cc(F)ccc1Sc1cc(-c2cn([C@H]3CCCNC3)nn2)cn2ncc(C#N)c12] |
| 461 | 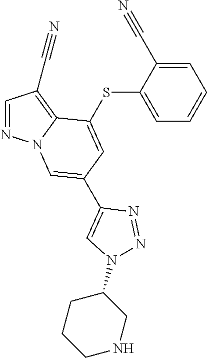 [SMILES: N#Cc1ccccc1Sc1cc(-c2cn([C@H]3CCCNC3)nn2)cn2ncc(C#N)c12] |
| 462 | 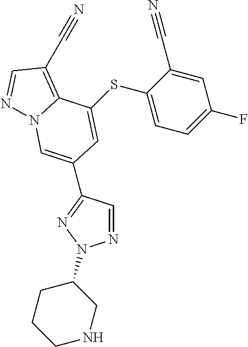 [SMILES: N#Cc1cc(F)ccc1Sc1cc(-c2cnn([C@H]3CCCNC3)n2)cn2ncc(C#N)c12] |
| 463 | 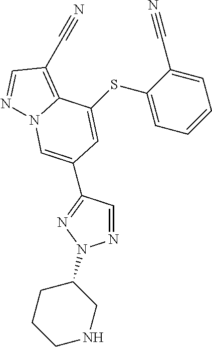 [SMILES: N#Cc1ccccc1Sc1cc(-c2cnn([C@H]3CCCNC3)n2)cn2ncc(C#N)c12] |
| 464 | 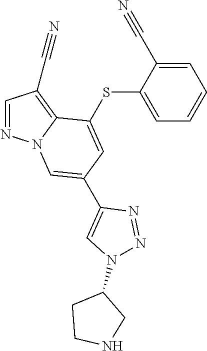 [SMILES: N#Cc1ccccc1Sc1cc(-c2cn([C@H]3CCNC3)nn2)cn2ncc(C#N)c12] |
| 465 | 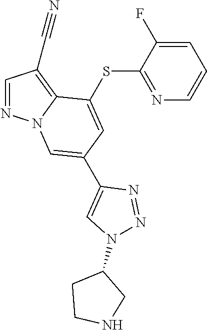 [SMILES: N#Cc1cnn2cc(-c3cn([C@H]4CCNC4)nn3)cc(Sc3ncccc3F)c12] |
| 466 | 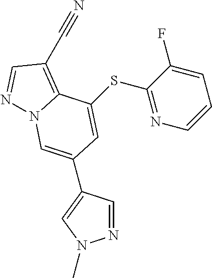 [SMILES: Cn1cc(-c2cc(Sc3ncccc3F)c3c(C#N)cnn3c2)cn1] |
| 467 | 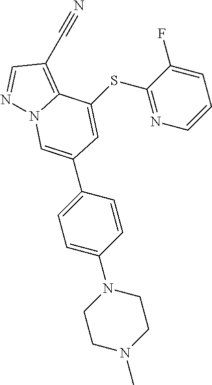 [SMILES: CN1CCN(c2ccc(-c3cc(Sc4ncccc4F)c4c(C#N)cnn4c3)cc2)CC1] |
| 468 | 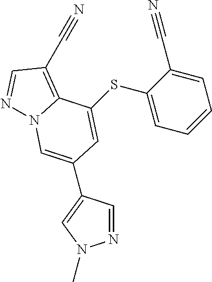 [SMILES: Cn1cc(-c2cc(Sc3ccccc3C#N)c3c(C#N)cnn3c2)cn1] |
| 469 | 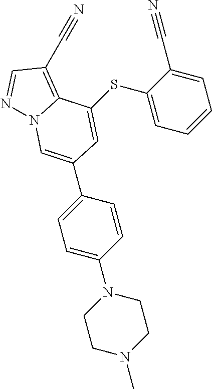 [SMILES: CN1CCN(c2ccc(-c3cc(Sc4ccccc4C#N)c4c(C#N)cnn4c3)cc2)CC1] |
| 470 | 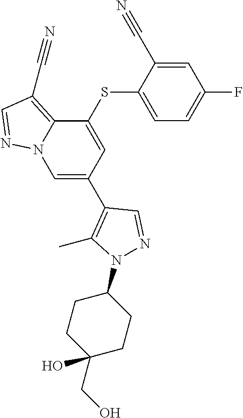 [SMILES: Cc1c(-c2cc(Sc3ccc(F)cc3C#N)c3c(C#N)cnn3c2)cnn1[C@H]1CC[C@](O)(CO)CC1] |
| 471 | 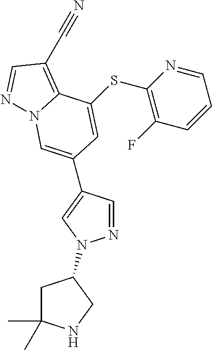 [SMILES: CC1(C)C[C@H](n2cc(-c3cc(Sc4ncccc4F)c4c(C#N)cnn4c3)cn2)CN1] |
| 472 | 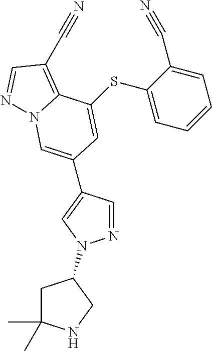 [SMILES: CC1(C)C[C@H](n2cc(-c3cc(Sc4ccccc4C#N)c4c(C#N)cnn4c3)cn2)CN1] |
| 473 | 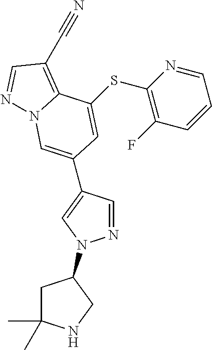 [SMILES: CC1(C)C[C@@H](n2cc(-c3cc(Sc4ncccc4F)c4c(C#N)cnn4c3)cn2)CN1] |
| 474 | 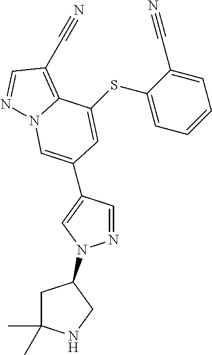 [SMILES: CC1(C)C[C@@H](n2cc(-c3cc(Sc4ccccc4C#N)c4c(C#N)cnn4c3)cn2)CN1] |
| 475 | 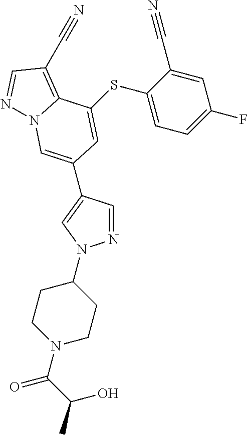 [SMILES: C[C@H](O)C(=O)N1CCC(n2cc(-c3cc(Sc4ccc(F)cc4C#N)c4c(C#N)cnn4c3)cn2)CC1] |
| 476 | 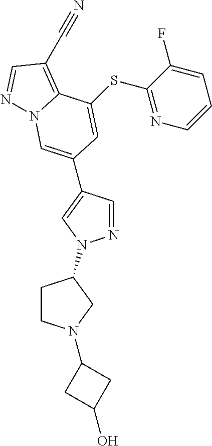 [SMILES: N#Cc1cnn2cc(-c3cnn([C@H]4CCN(C5CC(O)C5)C4)c3)cc(Sc3ncccc3F)c12] |
| 477 | 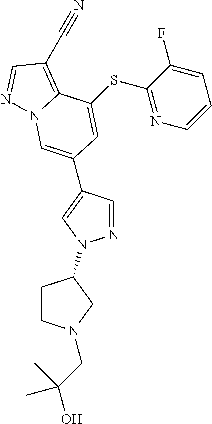 [SMILES: CC(C)(O)CN1CC[C@H](n2cc(-c3cc(Sc4ncccc4F)c4c(C#N)cnn4c3)cn2)C1] |
| 478 | 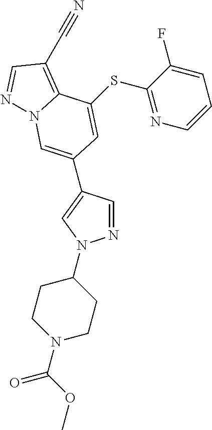 [SMILES: COC(=O)N1CCC(n2cc(-c3cc(Sc4ncccc4F)c4c(C#N)cnn4c3)cn2)CC1] |
| 479 | 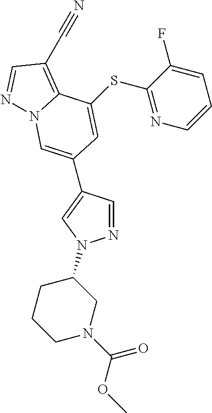 [SMILES: COC(=O)N1CCC[C@H](n2cc(-c3cc(Sc4ncccc4F)c4c(C#N)cnn4c3)cn2)C1] |
| 480 | 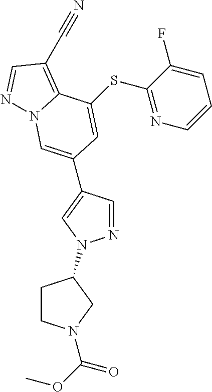 [SMILES: COC(=O)N1CC[C@H](n2cc(-c3cc(Sc4ncccc4F)c4c(C#N)cnn4c3)cn2)C1] |
| 481 | 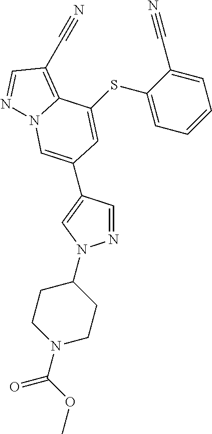 [SMILES: COC(=O)N1CCC(n2cc(-c3cc(Sc4ccccc4C#N)c4c(C#N)cnn4c3)cn2)CC1] |
| 482 | 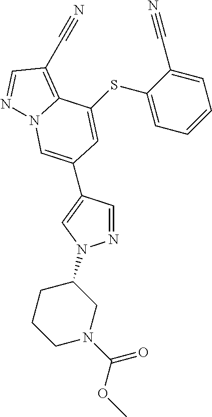 [SMILES: COC(=O)N1CCC[C@H](n2cc(-c3cc(Sc4ccccc4C#N)c4c(C#N)cnn4c3)cn2)C1] |
| 483 | 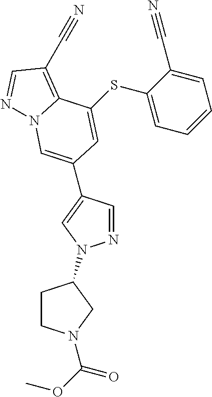 [SMILES: COC(=O)N1CC[C@H](n2cc(-c3cc(Sc4ccccc4C#N)c4c(C#N)cnn4c3)cn2)C1] |
| 484 | 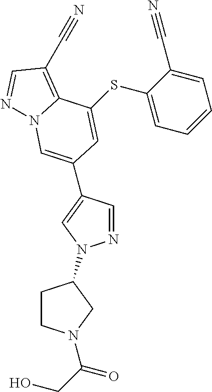 [SMILES: N#Cc1ccccc1Sc1cc(-c2cnn([C@H]3CCN(C(=O)CO)C3)c2)cn2ncc(C#N)c12] |
| 485 | 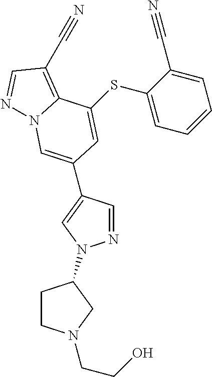 [SMILES: N#Cc1ccccc1Sc1cc(-c2cnn([C@H]3CCN(CCO)C3)c2)cn2ncc(C#N)c12] |
| 486 | 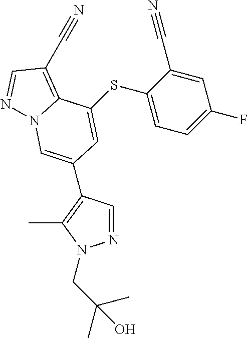 [SMILES: Cc1c(-c2cc(Sc3ccc(F)cc3C#N)c3c(C#N)cnn3c2)cnn1CC(C)(C)O] |
| 487 | 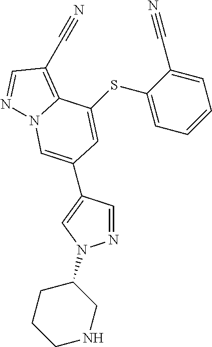 [SMILES: N#Cc1ccccc1Sc1cc(-c2cnn([C@H]3CCCNC3)c2)cn2ncc(C#N)c12] |
| 488 | 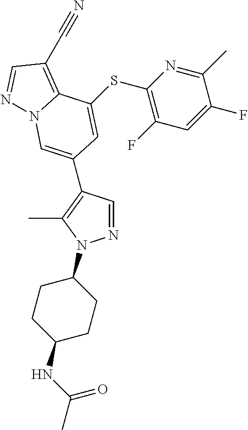 [SMILES: CC(=O)N[C@H]1CC[C@@H](n2ncc(-c3cc(Sc4nc(C)c(F)cc4F)c4c(C#N)cnn4c3)c2C)CC1] |
| 489 | 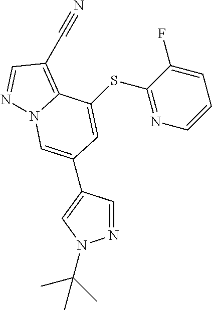 [SMILES: CC(C)(C)n1cc(-c2cc(Sc3ncccc3F)c3c(C#N)cnn3c2)cn1] |
| 490 | 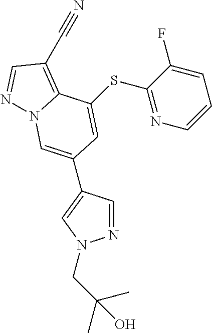 [SMILES: CC(C)(O)Cn1cc(-c2cc(Sc3ncccc3F)c3c(C#N)cnn3c2)cn1] |
| 491 | 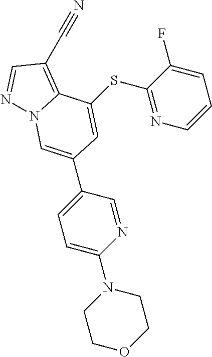 [SMILES: N#Cc1cnn2cc(-c3ccc(N4CCOCC4)nc3)cc(Sc3ncccc3F)c12] |
| 492 | 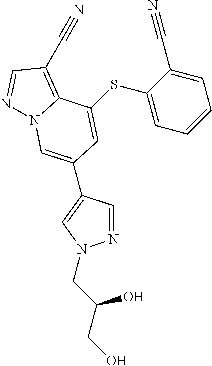 [SMILES: N#Cc1ccccc1Sc1cc(-c2cnn(C[C@@H](O)CO)c2)cn2ncc(C#N)c12] |
| 493 | 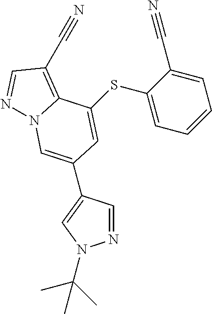 [SMILES: CC(C)(C)n1cc(-c2cc(Sc3ccccc3C#N)c3c(C#N)cnn3c2)cn1] |
| 494 | 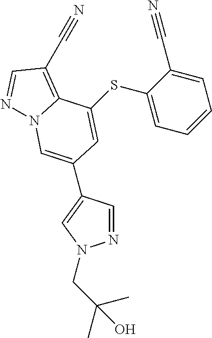 [SMILES: CC(C)(O)Cn1cc(-c2cc(Sc3ccccc3C#N)c3c(C#N)cnn3c2)cn1] |
| 495 | 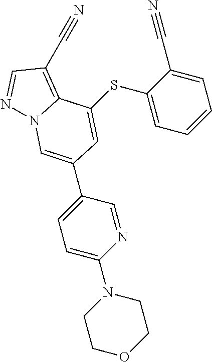 [SMILES: N#Cc1ccccc1Sc1cc(-c2ccc(N3CCOCC3)nc2)cn2ncc(C#N)c12] |
| 496 | 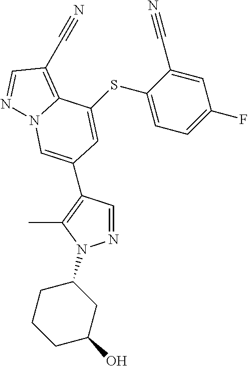 [SMILES: Cc1c(-c2cc(Sc3ccc(F)cc3C#N)c3c(C#N)cnn3c2)cnn1[C@H]1CCC[C@H](O)C1] |
| 497 | 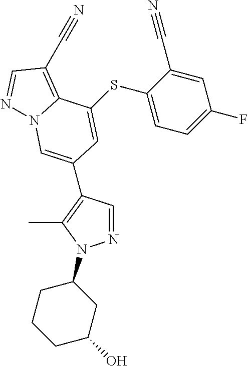 [SMILES: Cc1c(-c2cc(Sc3ccc(F)cc3C#N)c3c(C#N)cnn3c2)cnn1[C@@H]1CCC[C@@H](O)C1] |
| 498 | 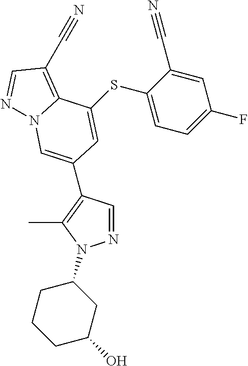 [SMILES: Cc1c(-c2cc(Sc3ccc(F)cc3C#N)c3c(C#N)cnn3c2)cnn1[C@H]1CCC[C@@H](O)C1] |
| 499 | 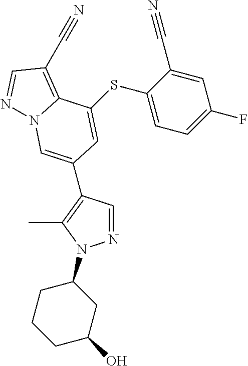 [SMILES: Cc1c(-c2cc(Sc3ccc(F)cc3C#N)c3c(C#N)cnn3c2)cnn1[C@@H]1CCC[C@H](O)C1] |
| 500 | 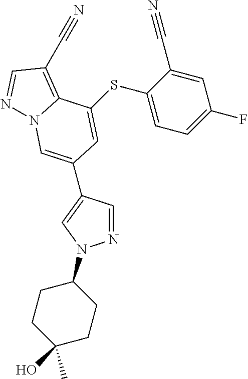 [SMILES: C[C@]1(O)CC[C@@H](n2cc(-c3cc(Sc4ccc(F)cc4C#N)c4c(C#N)cnn4c3)cn2)CC1] |
| 501 | 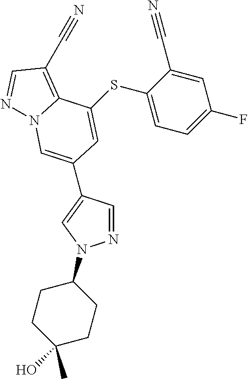 [SMILES: C[C@]1(O)CC[C@H](n2cc(-c3cc(Sc4ccc(F)cc4C#N)c4c(C#N)cnn4c3)cn2)CC1] |
| 502 | 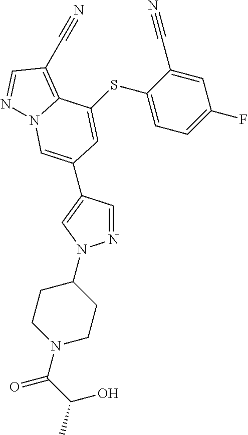 [SMILES: C[C@@H](O)C(=O)N1CCC(n2cc(-c3cc(Sc4ccc(F)cc4C#N)c4c(C#N)cnn4c3)cn2)CC1] |
| 503 | 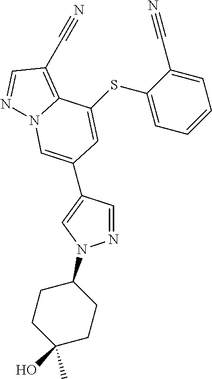 [SMILES: C[C@]1(O)CC[C@@H](n2cc(-c3cc(Sc4ccccc4C#N)c4c(C#N)cnn4c3)cn2)CC1] |
| 504 | 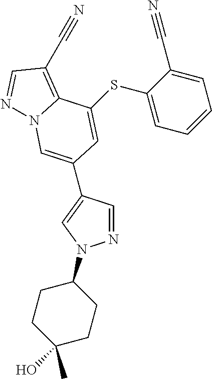 [SMILES: C[C@]1(O)CC[C@H](n2cc(-c3cc(Sc4ccccc4C#N)c4c(C#N)cnn4c3)cn2)CC1] |
| 505 | 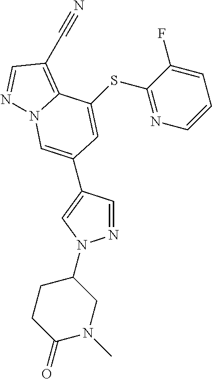 [SMILES: CN1CC(n2cc(-c3cc(Sc4ncccc4F)c4c(C#N)cnn4c3)cn2)CCC1=O] |
| 506 | 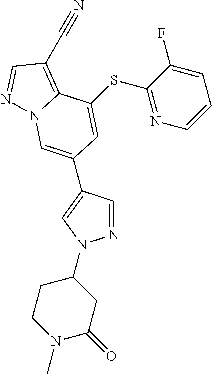 [SMILES: CN1CCC(n2cc(-c3cc(Sc4ncccc4F)c4c(C#N)cnn4c3)cn2)CC1=O] |
| 507 | 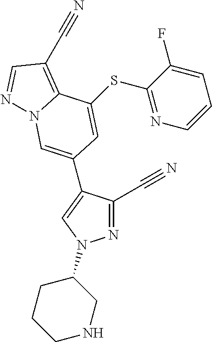 [SMILES: N#Cc1nn([C@H]2CCCNC2)cc1-c1cc(Sc2ncccc2F)c2c(C#N)cnn2c1] |
| 508 | 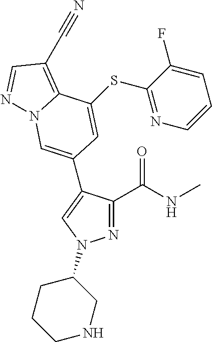 [SMILES: CNC(=O)c1nn([C@H]2CCCNC2)cc1-c1cc(Sc2ncccc2F)c2c(C#N)cnn2c1] |
| 509 | 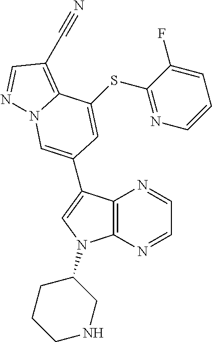 [SMILES: N#Cc1cnn2cc(-c3cn([C@H]4CCCNC4)c4nccnc34)cc(Sc3ncccc3F)c12] |
| 510 | 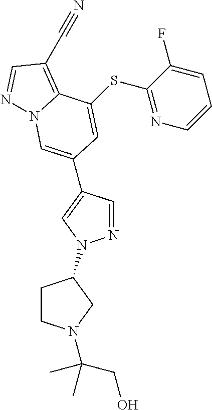 [SMILES: CC(C)(CO)N1CC[C@H](n2cc(-c3cc(Sc4ncccc4F)c4c(C#N)cnn4c3)cn2)C1] |
| 511 | 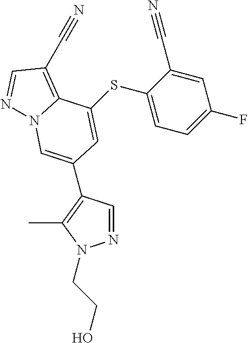 [SMILES: Cc1c(-c2cc(Sc3ccc(F)cc3C#N)c3c(C#N)cnn3c2)cnn1CCO] |
| 512 | 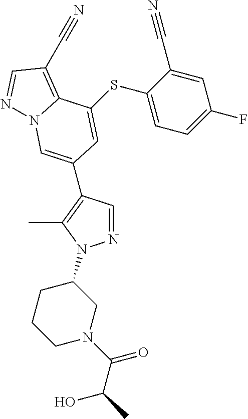 [SMILES: Cc1c(-c2cc(Sc3ccc(F)cc3C#N)c3c(C#N)cnn3c2)cnn1[C@H]1CCCN(C(=O)[C@@H](C)O)C1] |
| 513 | 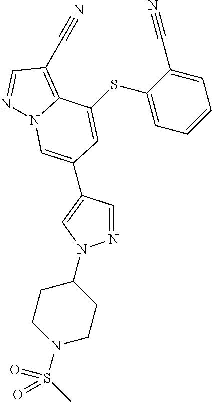 [SMILES: CS(=O)(=O)N1CCC(n2cc(-c3cc(Sc4ccccc4C#N)c4c(C#N)cnn4c3)cn2)CC1] |
| 514 | 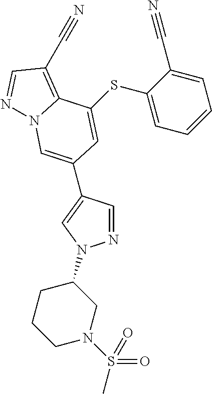 [SMILES: CS(=O)(=O)N1CCC[C@H](n2cc(-c3cc(Sc4ccccc4C#N)c4c(C#N)cnn4c3)cn2)C1] |
| 515 | 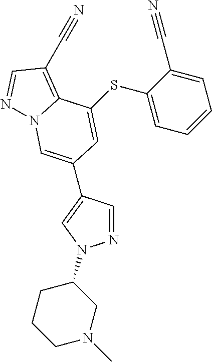 [SMILES: CN1CCC[C@H](n2cc(-c3cc(Sc4ccccc4C#N)c4c(C#N)cnn4c3)cn2)C1] |
| 516 | 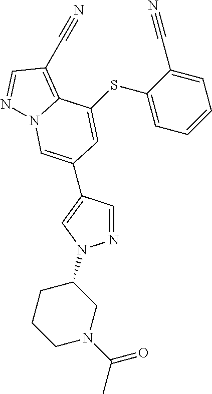 [SMILES: CC(=O)N1CCC[C@H](n2cc(-c3cc(Sc4ccccc4C#N)c4c(C#N)cnn4c3)cn2)C1] |
| 517 | 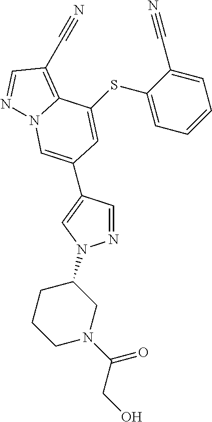 [SMILES: N#Cc1ccccc1Sc1cc(-c2cnn([C@H]3CCCN(C(=O)CO)C3)c2)cn2ncc(C#N)c12] |
| 518 | 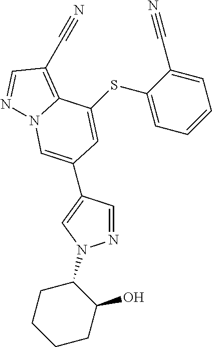 [SMILES: N#Cc1ccccc1Sc1cc(-c2cnn([C@H]3CCCC[C@@H]3O)c2)cn2ncc(C#N)c12] |
| 519 | 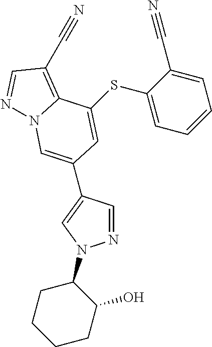 [SMILES: N#Cc1ccccc1Sc1cc(-c2cnn([C@@H]3CCCC[C@H]3O)c2)cn2ncc(C#N)c12] |
| 520 | 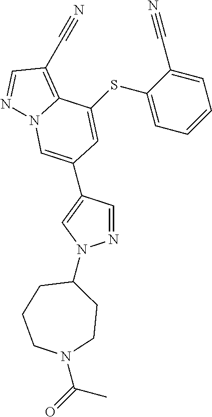 [SMILES: CC(=O)N1CCCC(n2cc(-c3cc(Sc4ccccc4C#N)c4c(C#N)cnn4c3)cn2)CC1] |
| 521 | 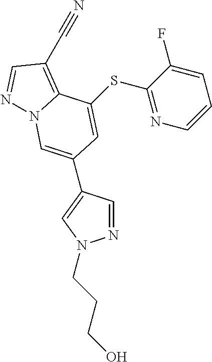 [SMILES: N#Cc1cnn2cc(-c3cnn(CCCO)c3)cc(Sc3ncccc3F)c12] |
| 522 | 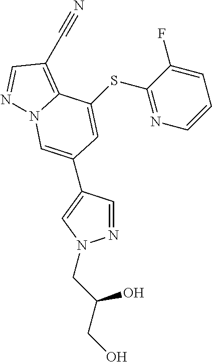 [SMILES: N#Cc1cnn2cc(-c3cnn(C[C@@H](O)CO)c3)cc(Sc3ncccc3F)c12] |
| 523 | 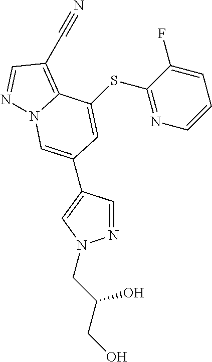 [SMILES: N#Cc1cnn2cc(-c3cnn(C[C@H](O)CO)c3)cc(Sc3ncccc3F)c12] |
| 524 | 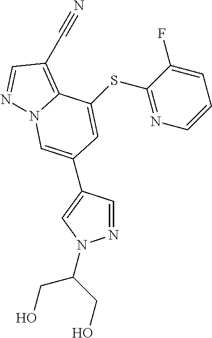 [SMILES: N#Cc1cnn2cc(-c3cnn(C(CO)CO)c3)cc(Sc3ncccc3F)c12] |
| 525 | 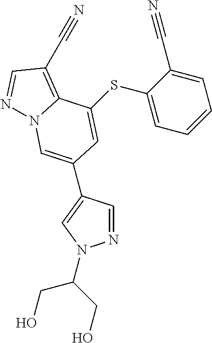 [SMILES: N#Cc1ccccc1Sc1cc(-c2cnn(C(CO)CO)c2)cn2ncc(C#N)c12] |
| 526 | 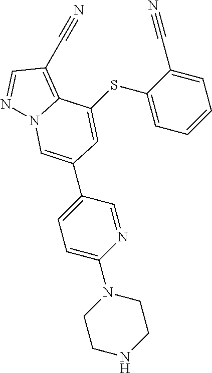 [SMILES: N#Cc1ccccc1Sc1cc(-c2ccc(N3CCNCC3)nc2)cn2ncc(C#N)c12] |
| 527 | 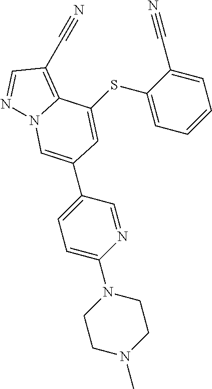 [SMILES: CN1CCN(c2ccc(-c3cc(Sc4ccccc4C#N)c4c(C#N)cnn4c3)cn2)CC1] |
| 528 | 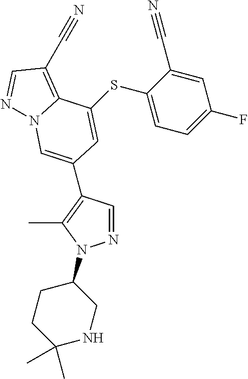 [SMILES: Cc1c(-c2cc(Sc3ccc(F)cc3C#N)c3c(C#N)cnn3c2)cnn1[C@@H]1CCC(C)(C)NC1] |
| 529 | 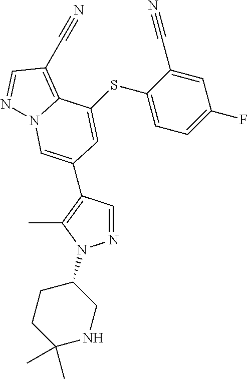 [SMILES: Cc1c(-c2cc(Sc3ccc(F)cc3C#N)c3c(C#N)cnn3c2)cnn1[C@H]1CCC(C)(C)NC1] |
| 530 | 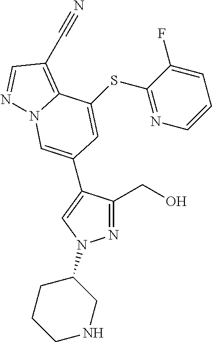 [SMILES: N#Cc1cnn2cc(-c3cn([C@H]4CCCNC4)nc3CO)cc(Sc3ncccc3F)c12] |
| 531 | 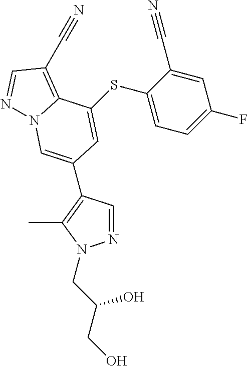 [SMILES: Cc1c(-c2cc(Sc3ccc(F)cc3C#N)c3c(C#N)cnn3c2)cnn1C[C@H](O)CO] |
| 532 | 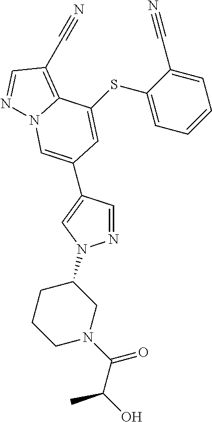 [SMILES: C[C@H](O)C(=O)N1CCC[C@H](n2cc(-c3cc(Sc4ccccc4C#N)c4c(C#N)cnn4c3)cn2)C1] |
| 533 | 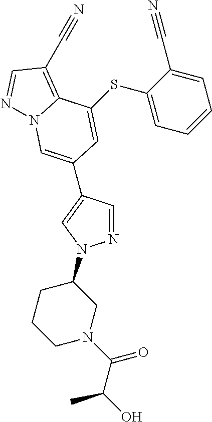 [SMILES: C[C@H](O)C(=O)N1CCC[C@@H](n2cc(-c3cc(Sc4ccccc4C#N)c4c(C#N)cnn4c3)cn2)C1] |
| 534 | 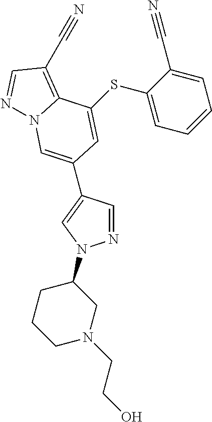 [SMILES: N#Cc1ccccc1Sc1cc(-c2cnn([C@@H]3CCCN(CCO)C3)c2)cn2ncc(C#N)c12] |
| 535 | 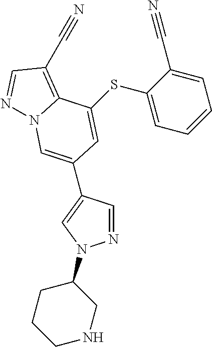 [SMILES: N#Cc1ccccc1Sc1cc(-c2cnn([C@@H]3CCCNC3)c2)cn2ncc(C#N)c12] |
| 536 | 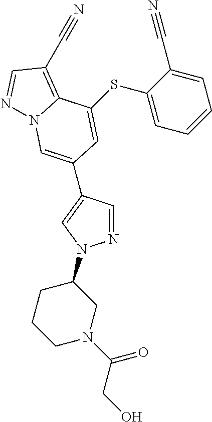 [SMILES: N#Cc1ccccc1Sc1cc(-c2cnn([C@@H]3CCCN(C(=O)CO)C3)c2)cn2ncc(C#N)c12] |
| 537 | 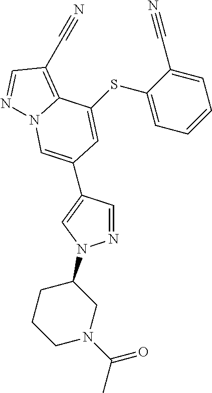 [SMILES: CC(=O)N1CCC[C@@H](n2cc(-c3cc(Sc4ccccc4C#N)c4c(C#N)cnn4c3)cn2)C1] |
| 538 | 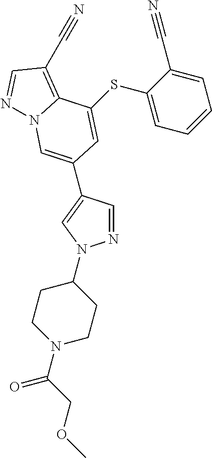 [SMILES: COCC(=O)N1CCC(n2cc(-c3cc(Sc4ccccc4C#N)c4c(C#N)cnn4c3)cn2)CC1] |
| 539 | 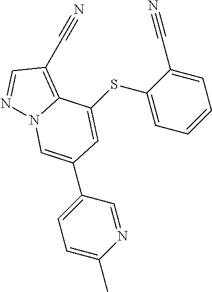 [SMILES: Cc1ccc(-c2cc(Sc3ccccc3C#N)c3c(C#N)cnn3c2)cn1] |
| 540 | 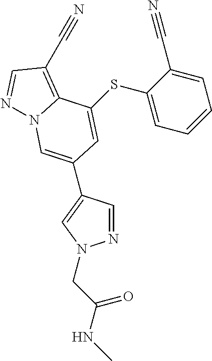 [SMILES: CNC(=O)Cn1cc(-c2cc(Sc3ccccc3C#N)c3c(C#N)cnn3c2)cn1] |
| 541 | 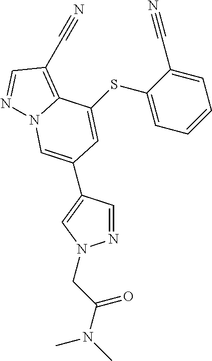 [SMILES: CN(C)C(=O)Cn1cc(-c2cc(Sc3ccccc3C#N)c3c(C#N)cnn3c2)cn1] |
| 542 | 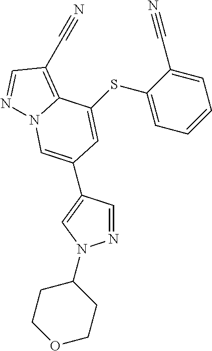 [SMILES: N#Cc1ccccc1Sc1cc(-c2cnn(C3CCOCC3)c2)cn2ncc(C#N)c12] |
| 543 | 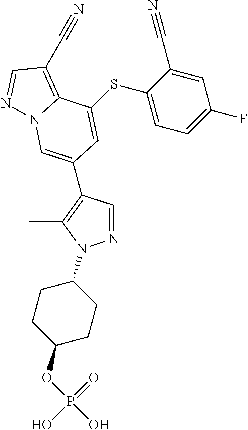 [SMILES: Cc1c(-c2cc(Sc3ccc(F)cc3C#N)c3c(C#N)cnn3c2)cnn1[C@H]1CC[C@H](OP(=O)(O)O)CC1] |
| 544 | 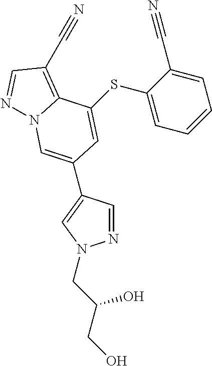 [SMILES: N#Cc1ccccc1Sc1cc(-c2cnn(C[C@H](O)CO)c2)cn2ncc(C#N)c12] |
| 545 | 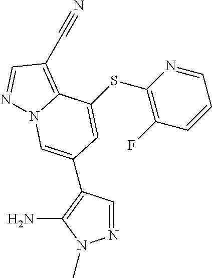 [SMILES: Cn1ncc(-c2cc(Sc3ncccc3F)c3c(C#N)cnn3c2)c1N] |
| 546 | 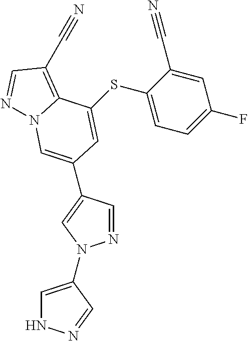 [SMILES: N#Cc1cc(F)ccc1Sc1cc(-c2cnn(-c3cn[nH]c3)c2)cn2ncc(C#N)c12] |
| 547 | 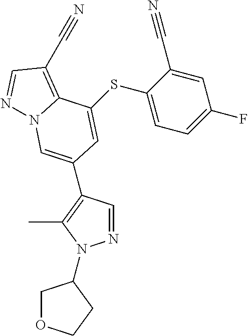 [SMILES: Cc1c(-c2cc(Sc3ccc(F)cc3C#N)c3c(C#N)cnn3c2)cnn1C1CCOC1] |
| 548 | 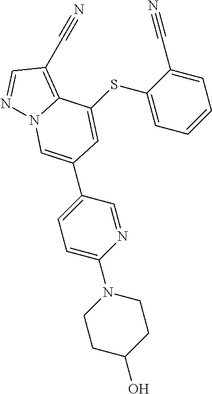 [SMILES: N#Cc1ccccc1Sc1cc(-c2ccc(N3CCC(O)CC3)nc2)cn2ncc(C#N)c12] |
| 549 | 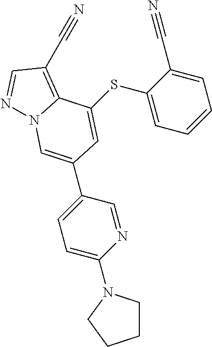 [SMILES: N#Cc1ccccc1Sc1cc(-c2ccc(N3CCCC3)nc2)cn2ncc(C#N)c12] |
| 550 | 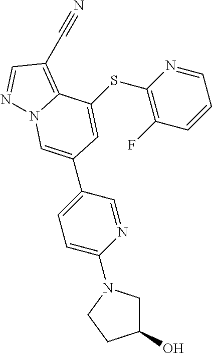 [SMILES: N#Cc1cnn2cc(-c3ccc(N4CC[C@H](O)C4)nc3)cc(Sc3ncccc3F)c12] |
| 551 | 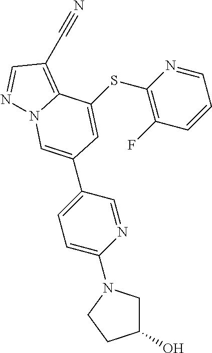 [SMILES: N#Cc1cnn2cc(-c3ccc(N4CC[C@@H](O)C4)nc3)cc(Sc3ncccc3F)c12] |
| 552 | 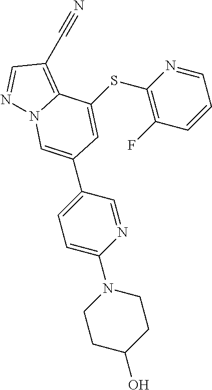 [SMILES: N#Cc1cnn2cc(-c3ccc(N4CCC(O)CC4)nc3)cc(Sc3ncccc3F)c12] |
| 553 | 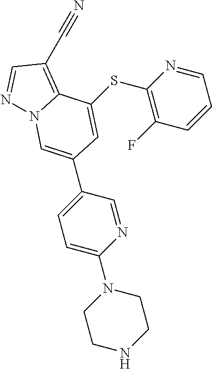 [SMILES: N#Cc1cnn2cc(-c3ccc(N4CCNCC4)nc3)cc(Sc3ncccc3F)c12] |
| 554 | 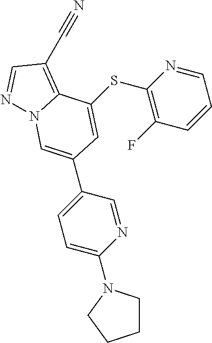 [SMILES: N#Cc1cnn2cc(-c3ccc(N4CCCC4)nc3)cc(Sc3ncccc3F)c12] |
| 555 | 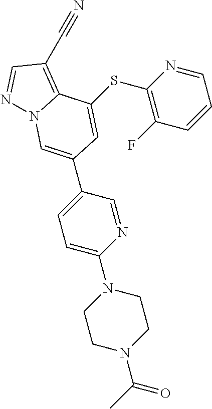 [SMILES: CC(=O)N1CCN(c2ccc(-c3cc(Sc4ncccc4F)c4c(C#N)cnn4c3)cn2)CC1] |
| 556 | 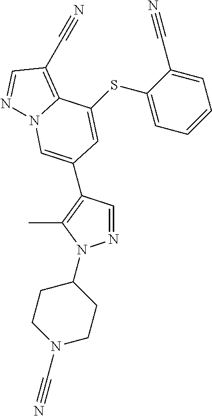 [SMILES: Cc1c(-c2cc(Sc3ccccc3C#N)c3c(C#N)cnn3c2)cnn1C1CCN(C#N)CC1] |
| 557 | 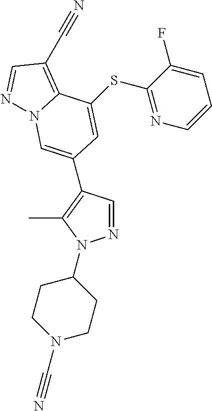 [SMILES: Cc1c(-c2cc(Sc3ncccc3F)c3c(C#N)cnn3c2)cnn1C1CCN(C#N)CC1] |
| 558 | 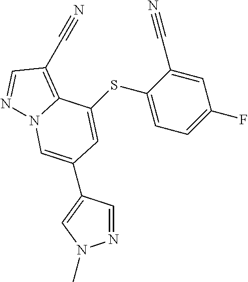 [SMILES: Cn1cc(-c2cc(Sc3ccc(F)cc3C#N)c3c(C#N)cnn3c2)cn1] |
| 559 | 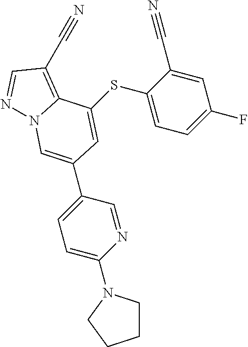 [SMILES: N#Cc1cc(F)ccc1Sc1cc(-c2ccc(N3CCCC3)nc2)cn2ncc(C#N)c12] |
| 560 | 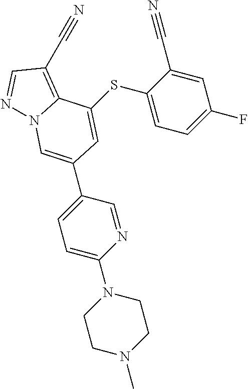 [SMILES: CN1CCN(c2ccc(-c3cc(Sc4ccc(F)cc4C#N)c4c(C#N)cnn4c3)cn2)CC1] |
| 561 | 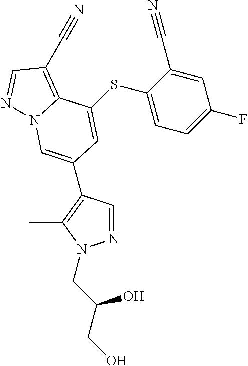 [SMILES: Cc1c(-c2cc(Sc3ccc(F)cc3C#N)c3c(C#N)cnn3c2)cnn1C[C@@H](O)CO] |
| 562 | 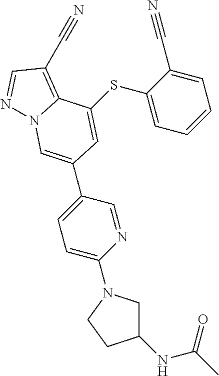 [SMILES: CC(=O)NC1CCN(c2ccc(-c3cc(Sc4ccccc4C#N)c4c(C#N)cnn4c3)cn2)C1] |
| 563 | 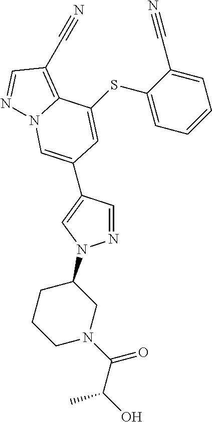 [SMILES: C[C@@H](O)C(=O)N1CCC[C@@H](n2cc(-c3cc(Sc4ccccc4C#N)c4c(C#N)cnn4c3)cn2)C1] |
| 564 | 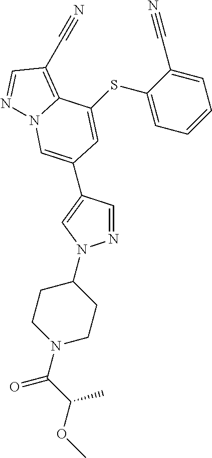 [SMILES: CO[C@@H](C)C(=O)N1CCC(n2cc(-c3cc(Sc4ccccc4C#N)c4c(C#N)cnn4c3)cn2)CC1] |
| 565 | 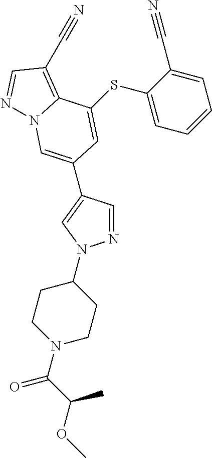 [SMILES: CO[C@H](C)C(=O)N1CCC(n2cc(-c3cc(Sc4ccccc4C#N)c4c(C#N)cnn4c3)cn2)CC1] |
| 566 | 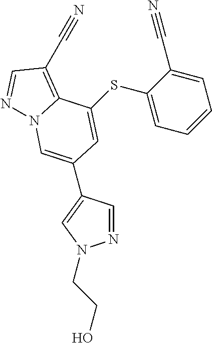 [SMILES: N#Cc1ccccc1Sc1cc(-c2cnn(CCO)c2)cn2ncc(C#N)c12] |
| 567 | 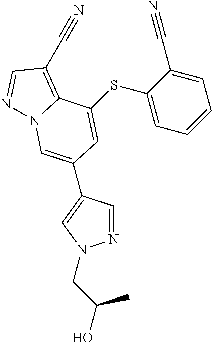 [SMILES: C[C@@H](O)Cn1cc(-c2cc(Sc3ccccc3C#N)c3c(C#N)cnn3c2)cn1] |
| 568 | 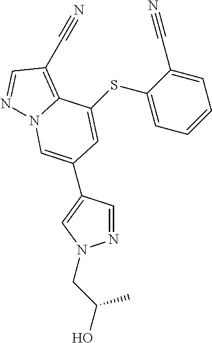 [SMILES: C[C@H](O)Cn1cc(-c2cc(Sc3ccccc3C#N)c3c(C#N)cnn3c2)cn1] |
| 569 | 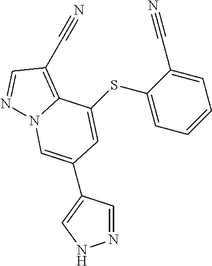 [SMILES: N#Cc1ccccc1Sc1cc(-c2cn[nH]c2)cn2ncc(C#N)c12] |
| 570 | 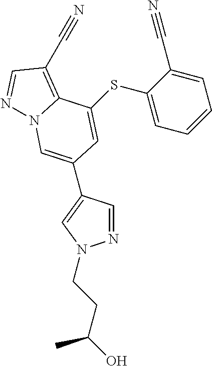 [SMILES: C[C@H](O)CCn1cc(-c2cc(Sc3ccccc3C#N)c3c(C#N)cnn3c2)cn1] |
| 571 | 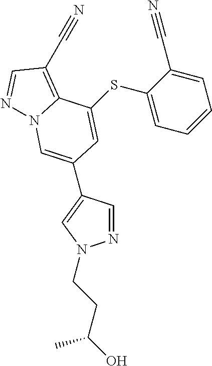 [SMILES: C[C@@H](O)CCn1cc(-c2cc(Sc3ccccc3C#N)c3c(C#N)cnn3c2)cn1] |
| 572 | 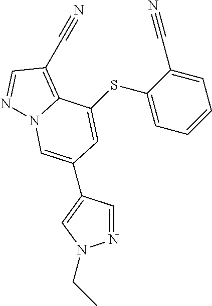 [SMILES: CCn1cc(-c2cc(Sc3ccccc3C#N)c3c(C#N)cnn3c2)cn1] |
| 573 | 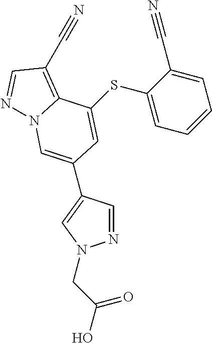 [SMILES: N#Cc1ccccc1Sc1cc(-c2cnn(CC(=O)O)c2)cn2ncc(C#N)c12] |
| 574 | 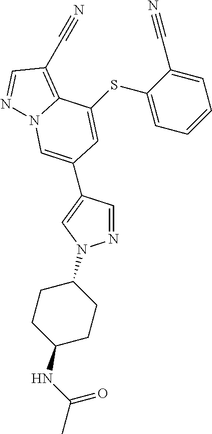 [SMILES: CC(=O)N[C@H]1CC[C@H](n2cc(-c3cc(Sc4ccccc4C#N)c4c(C#N)cnn4c3)cn2)CC1] |
| 575 | 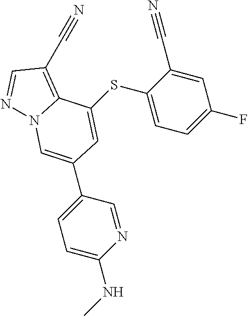 [SMILES: CNc1ccc(-c2cc(Sc3ccc(F)cc3C#N)c3c(C#N)cnn3c2)cn1] |
| 576 | 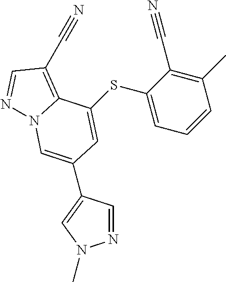 [SMILES: Cc1cccc(Sc2cc(-c3cnn(C)c3)cn3ncc(C#N)c23)c1C#N] |
| 577 | 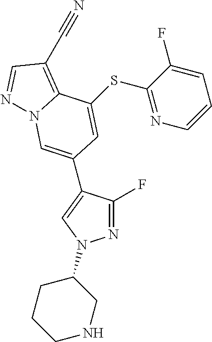 [SMILES: N#Cc1cnn2cc(-c3cn([C@H]4CCCNC4)nc3F)cc(Sc3ncccc3F)c12] |
| 578 | 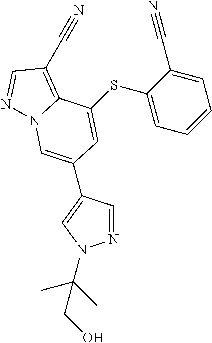 [SMILES: CC(C)(CO)n1cc(-c2cc(Sc3ccccc3C#N)c3c(C#N)cnn3c2)cn1] |
| 579 | 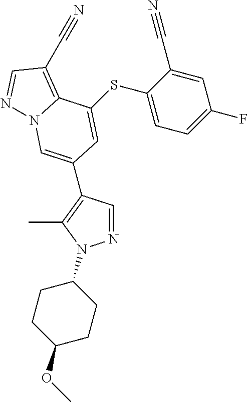 [SMILES: CO[C@H]1CC[C@H](n2ncc(-c3cc(Sc4ccc(F)cc4C#N)c4c(C#N)cnn4c3)c2C)CC1] |
| 580 | 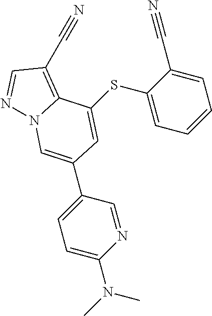 [SMILES: CN(C)c1ccc(-c2cc(Sc3ccccc3C#N)c3c(C#N)cnn3c2)cn1] |
| 581 | 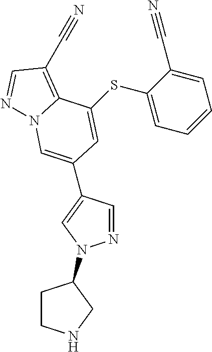 [SMILES: N#Cc1ccccc1Sc1cc(-c2cnn([C@@H]3CCNC3)c2)cn2ncc(C#N)c12] |
| 582 | 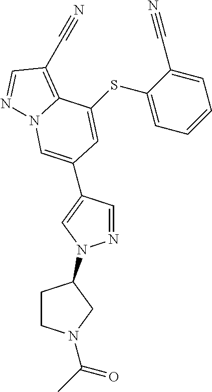 [SMILES: CC(=O)N1CC[C@@H](n2cc(-c3cc(Sc4ccccc4C#N)c4c(C#N)cnn4c3)cn2)C1] |
| 583 | 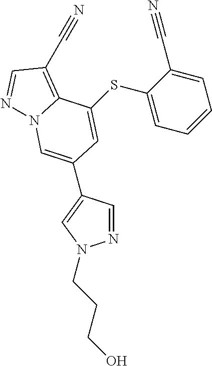 [SMILES: N#Cc1ccccc1Sc1cc(-c2cnn(CCCO)c2)cn2ncc(C#N)c12] |
| 584 | 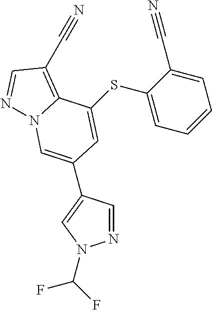 [SMILES: N#Cc1ccccc1Sc1cc(-c2cnn(C(F)F)c2)cn2ncc(C#N)c12] |
| 585 | 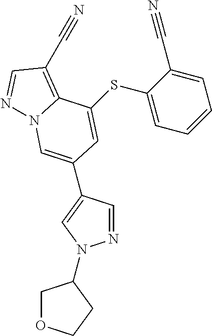 [SMILES: N#Cc1ccccc1Sc1cc(-c2cnn(C3CCOC3)c2)cn2ncc(C#N)c12] |
| 586 | 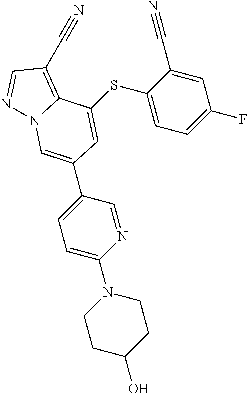 [SMILES: N#Cc1cc(F)ccc1Sc1cc(-c2ccc(N3CCC(O)CC3)nc2)cn2ncc(C#N)c12] |
| 587 | 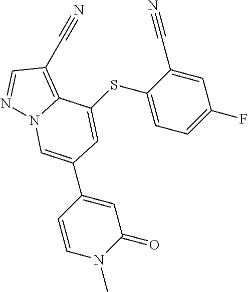 [SMILES: Cn1ccc(-c2cc(Sc3ccc(F)cc3C#N)c3c(C#N)cnn3c2)cc1=O] |
| 588 | 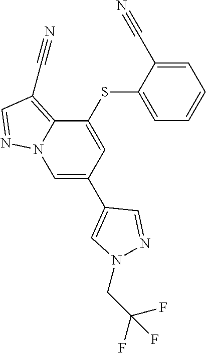 [SMILES: N#Cc1ccccc1Sc1cc(-c2cnn(CC(F)(F)F)c2)cn2ncc(C#N)c12] |
| 589 | 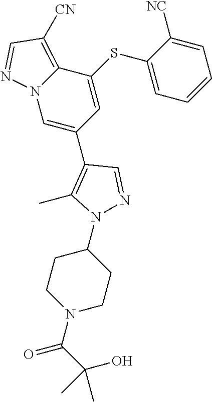 [SMILES: Cc1c(-c2cc(Sc3ccccc3C#N)c3c(C#N)cnn3c2)cnn1C1CCN(C(=O)C(C)(C)O)CC1] |
| 590 | 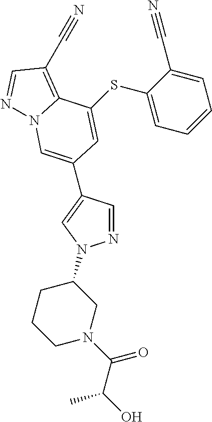 [SMILES: C[C@@H](O)C(=O)N1CCC[C@H](n2cc(-c3cc(Sc4ccccc4C#N)c4c(C#N)cnn4c3)cn2)C1] |
| 591 | 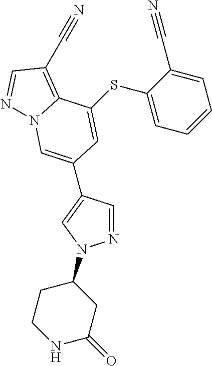 [SMILES: N#Cc1ccccc1Sc1cc(-c2cnn([C@@H]3CCNC(=O)C3)c2)cn2ncc(C#N)c12] |
| 592 | 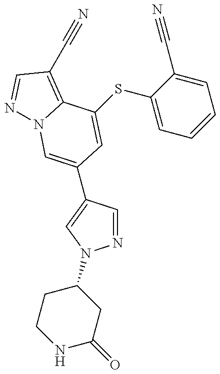 [SMILES: N#Cc1ccccc1Sc1cc(-c2cnn([C@H]3CCNC(=O)C3)c2)cn2ncc(C#N)c12] |
| 593 | 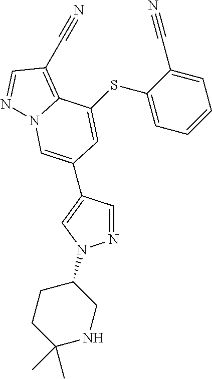 [SMILES: CC1(C)CC[C@H](n2cc(-c3cc(Sc4ccccc4C#N)c4c(C#N)cnn4c3)cn2)CN1] |
| 594 | 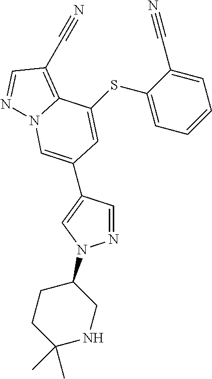 [SMILES: CC1(C)CC[C@@H](n2cc(-c3cc(Sc4ccccc4C#N)c4c(C#N)cnn4c3)cn2)CN1] |
| 595 | 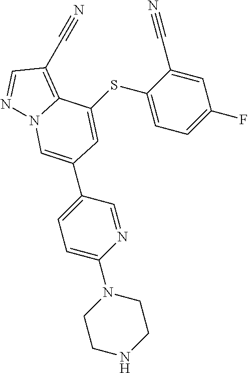 [SMILES: N#Cc1cc(F)ccc1Sc1cc(-c2ccc(N3CCNCC3)nc2)cn2ncc(C#N)c12] |
| 596 | 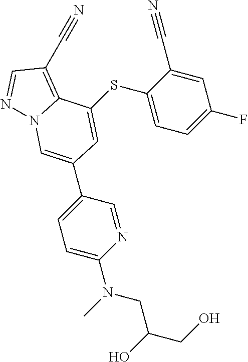 [SMILES: CN(CC(O)CO)c1ccc(-c2cc(Sc3ccc(F)cc3C#N)c3c(C#N)cnn3c2)cn1] |
| 597 | 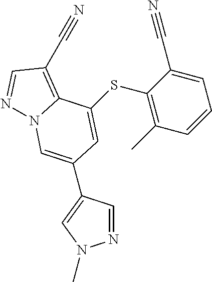 [SMILES: Cc1cccc(C#N)c1Sc1cc(-c2cnn(C)c2)cn2ncc(C#N)c12] |
| 598 | 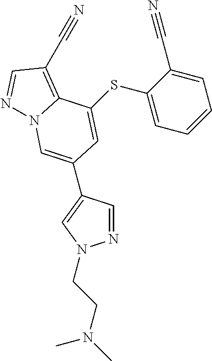 [SMILES: CN(C)CCn1cc(-c2cc(Sc3ccccc3C#N)c3c(C#N)cnn3c2)cn1] |
| 599 | 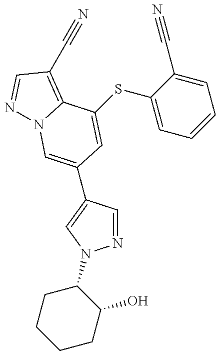 [SMILES: N#Cc1ccccc1Sc1cc(-c2cnn([C@H]3CCCC[C@H]3O)c2)cn2ncc(C#N)c12] |
| 600 | 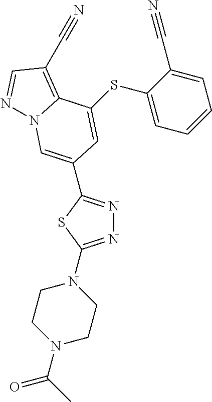 [SMILES: CC(=O)N1CCN(c2nnc(-c3cc(Sc4ccccc4C#N)c4c(C#N)cnn4c3)s2)CC1] |
| 601 | 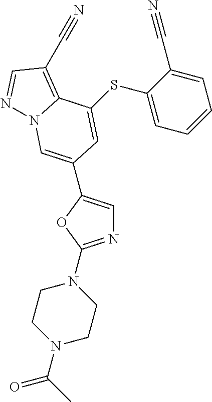 [SMILES: CC(=O)N1CCN(c2ncc(-c3cc(Sc4ccccc4C#N)c4c(C#N)cnn4c3)o2)CC1] |
| 602 | 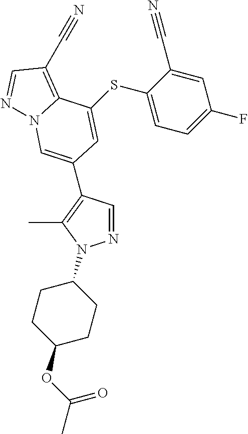 [SMILES: CC(=O)O[C@H]1CC[C@H](n2ncc(-c3cc(Sc4ccc(F)cc4C#N)c4c(C#N)cnn4c3)c2C)CC1] |
| 603 | 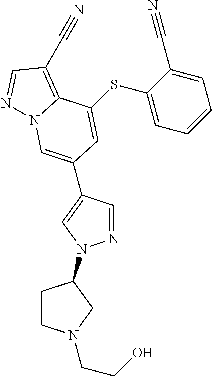 [SMILES: N#Cc1ccccc1Sc1cc(-c2cnn([C@@H]3CCN(CCO)C3)c2)cn2ncc(C#N)c12] |
| 604 | 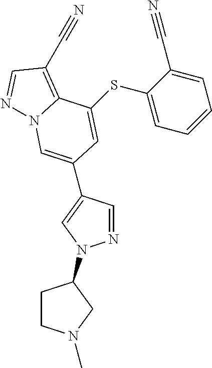 [SMILES: CN1CC[C@@H](n2cc(-c3cc(Sc4ccccc4C#N)c4c(C#N)cnn4c3)cn2)C1] |
| 605 | 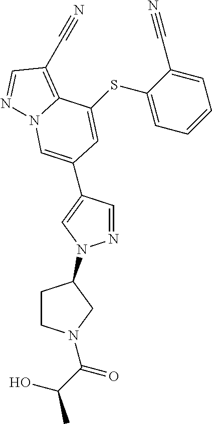 [SMILES: C[C@@H](O)C(=O)N1CC[C@@H](n2cc(-c3cc(Sc4ccccc4C#N)c4c(C#N)cnn4c3)cn2)C1] |
| 606 | 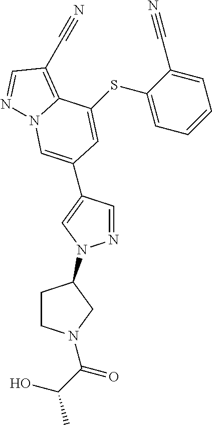 [SMILES: C[C@H](O)C(=O)N1CC[C@@H](n2cc(-c3cc(Sc4ccccc4C#N)c4c(C#N)cnn4c3)cn2)C1] |
| 607 | 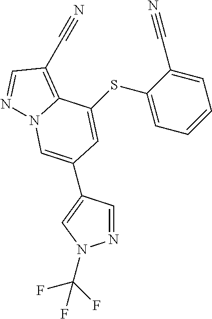 [SMILES: N#Cc1ccccc1Sc1cc(-c2cnn(C(F)(F)F)c2)cn2ncc(C#N)c12] |
| 608 | 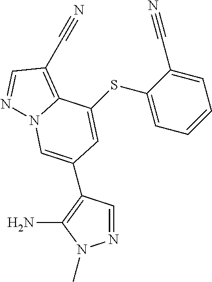 [SMILES: Cn1ncc(-c2cc(Sc3ccccc3C#N)c3c(C#N)cnn3c2)c1N] |
| 609 | 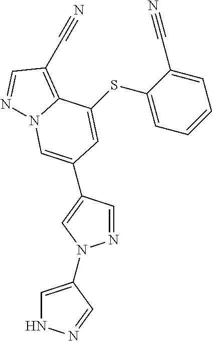 [SMILES: N#Cc1ccccc1Sc1cc(-c2cnn(-c3cn[nH]c3)c2)cn2ncc(C#N)c12] |
| 610 | 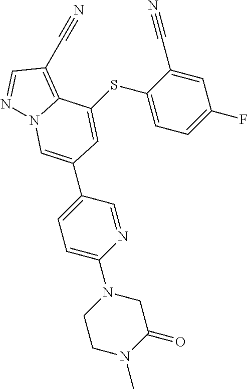 [SMILES: CN1CCN(c2ccc(-c3cc(Sc4ccc(F)cc4C#N)c4c(C#N)cnn4c3)cn2)CC1=O] |
| 611 | 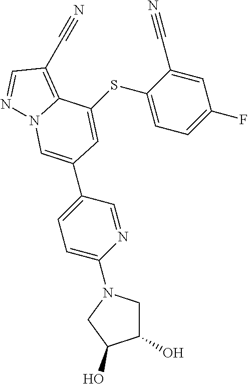 [SMILES: N#Cc1cc(F)ccc1Sc1cc(-c2ccc(N3C[C@H](O)[C@@H](O)C3)nc2)cn2ncc(C#N)c12] |
| 612 | 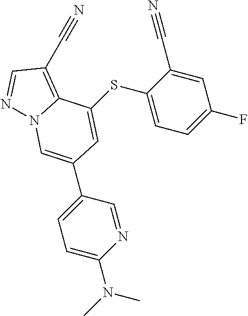 [SMILES: CN(C)c1ccc(-c2cc(Sc3ccc(F)cc3C#N)c3c(C#N)cnn3c2)cn1] |
| 613 | 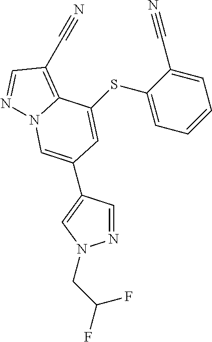 [SMILES: N#Cc1ccccc1Sc1cc(-c2cnn(CC(F)F)c2)cn2ncc(C#N)c12] |
| 614 | 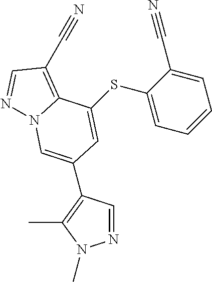 [SMILES: Cc1c(-c2cc(Sc3ccccc3C#N)c3c(C#N)cnn3c2)cnn1C] |
| 615 | 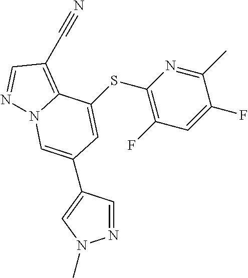 [SMILES: Cc1nc(Sc2cc(-c3cnn(C)c3)cn3ncc(C#N)c23)c(F)cc1F] |
| 616 | 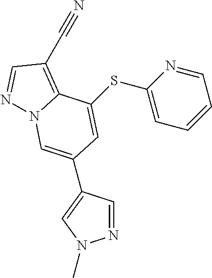 [SMILES: Cn1cc(-c2cc(Sc3ccccn3)c3c(C#N)cnn3c2)cn1] |
| 617 | 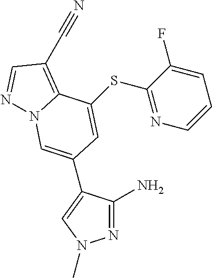 [SMILES: Cn1cc(-c2cc(Sc3ncccc3F)c3c(C#N)cnn3c2)c(N)n1] |
| 618 | 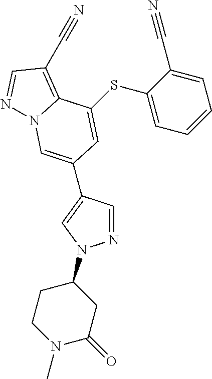 [SMILES: CN1CC[C@@H](n2cc(-c3cc(Sc4ccccc4C#N)c4c(C#N)cnn4c3)cn2)CC1=O] |
| 619 | 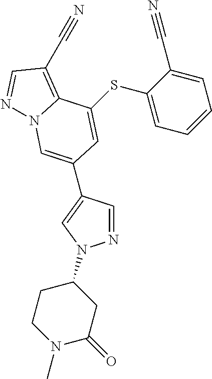 [SMILES: CN1CC[C@H](n2cc(-c3cc(Sc4ccccc4C#N)c4c(C#N)cnn4c3)cn2)CC1=O] |
| 620 | 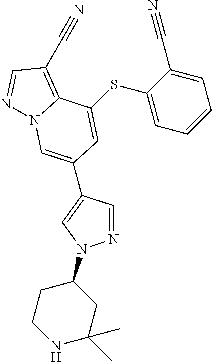 [SMILES: CC1(C)C[C@H](n2cc(-c3cc(Sc4ccccc4C#N)c4c(C#N)cnn4c3)cn2)CCN1] |
| 621 | 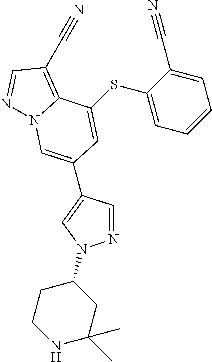 [SMILES: CC1(C)C[C@@H](n2cc(-c3cc(Sc4ccccc4C#N)c4c(C#N)cnn4c3)cn2)CCN1] |
| 622 | 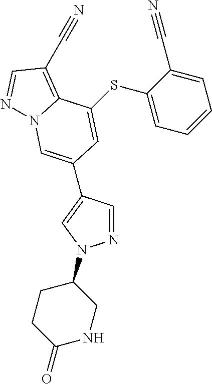 [SMILES: N#Cc1ccccc1Sc1cc(-c2cnn([C@@H]3CCC(=O)NC3)c2)cn2ncc(C#N)c12] |
| 623 | 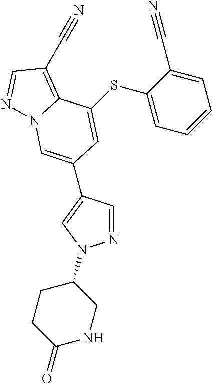 [SMILES: N#Cc1ccccc1Sc1cc(-c2cnn([C@H]3CCC(=O)NC3)c2)cn2ncc(C#N)c12] |
| 624 | 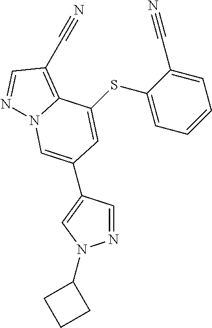 [SMILES: N#Cc1ccccc1Sc1cc(-c2cnn(C3CCC3)c2)cn2ncc(C#N)c12] |
| 625 | 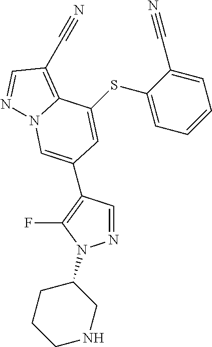 [SMILES: N#Cc1ccccc1Sc1cc(-c2cnn([C@H]3CCCNC3)c2F)cn2ncc(C#N)c12] |
| 626 | 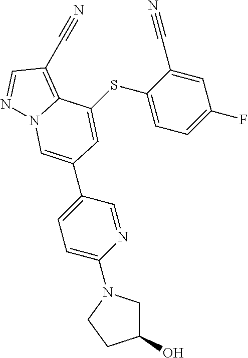 [SMILES: N#Cc1cc(F)ccc1Sc1cc(-c2ccc(N3CC[C@H](O)C3)nc2)cn2ncc(C#N)c12] |
| 627 | 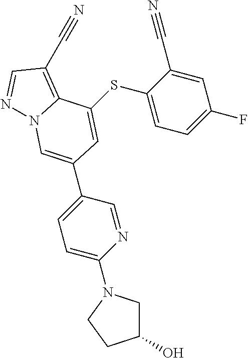 [SMILES: N#Cc1cc(F)ccc1Sc1cc(-c2ccc(N3CC[C@@H](O)C3)nc2)cn2ncc(C#N)c12] |
| 628 | 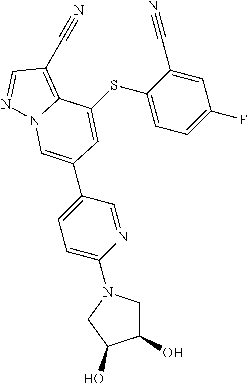 [SMILES: N#Cc1cc(F)ccc1Sc1cc(-c2ccc(N3C[C@@H](O)[C@@H](O)C3)nc2)cn2ncc(C#N)c12] |
| 629 | 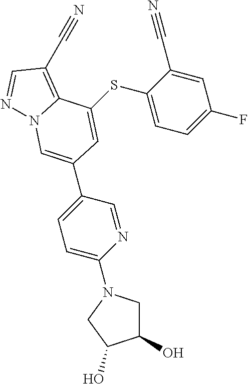 [SMILES: N#Cc1cc(F)ccc1Sc1cc(-c2ccc(N3C[C@@H](O)[C@H](O)C3)nc2)cn2ncc(C#N)c12] |
| 630 | 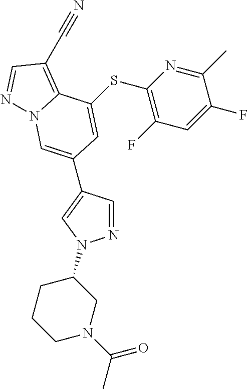 [SMILES: CC(=O)N1CCC[C@H](n2cc(-c3cc(Sc4nc(C)c(F)cc4F)c4c(C#N)cnn4c3)cn2)C1] |
| 631 | 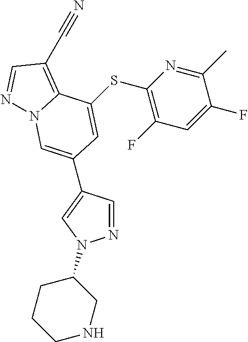 [SMILES: Cc1nc(Sc2cc(-c3cnn([C@H]4CCCNC4)c3)cn3ncc(C#N)c23)c(F)cc1F] |
| 632 | 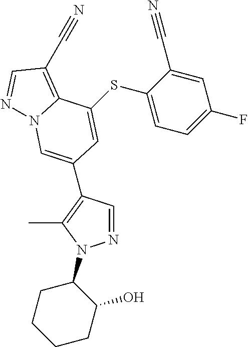 [SMILES: Cc1c(-c2cc(Sc3ccc(F)cc3C#N)c3c(C#N)cnn3c2)cnn1[C@@H]1CCCC[C@H]1O] |
| 633 | 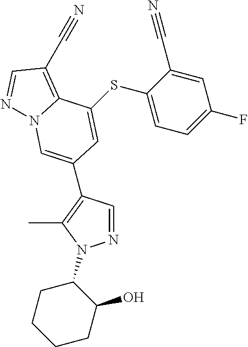 [SMILES: Cc1c(-c2cc(Sc3ccc(F)cc3C#N)c3c(C#N)cnn3c2)cnn1[C@H]1CCCC[C@@H]1O] |
| 634 | 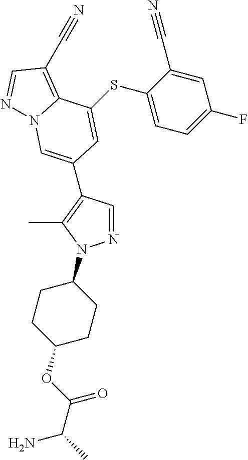 [SMILES: Cc1c(-c2cc(Sc3ccc(F)cc3C#N)c3c(C#N)cnn3c2)cnn1[C@H]1CC[C@H](OC(=O)[C@H](C)N)CC1] |
| 635 | 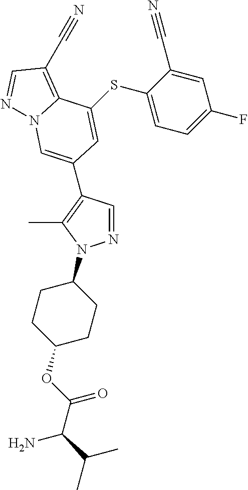 [SMILES: Cc1c(-c2cc(Sc3ccc(F)cc3C#N)c3c(C#N)cnn3c2)cnn1[C@H]1CC[C@H](OC(=O)[C@H](N)C(C)C)CC1] |
| 636 | 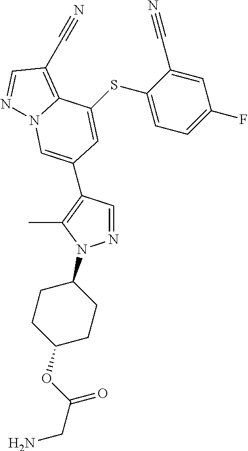 [SMILES: Cc1c(-c2cc(Sc3ccc(F)cc3C#N)c3c(C#N)cnn3c2)cnn1[C@H]1CC[C@H](OC(=O)CN)CC1] |
| 637 | 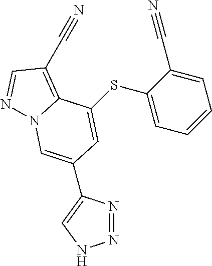 [SMILES: N#Cc1ccccc1Sc1cc(-c2c[nH]nn2)cn2ncc(C#N)c12] |
| 638 | 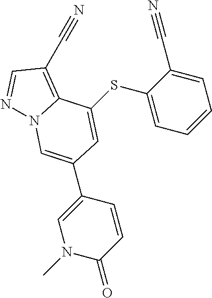 [SMILES: Cn1cc(-c2cc(Sc3ccccc3C#N)c3c(C#N)cnn3c2)ccc1=O] |
| 639 | 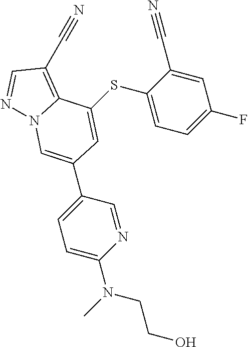 [SMILES: CN(CCO)c1ccc(-c2cc(Sc3ccc(F)cc3C#N)c3c(C#N)cnn3c2)cn1] |
| 640 | 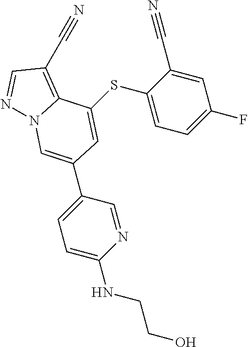 [SMILES: N#Cc1cc(F)ccc1Sc1cc(-c2ccc(NCCO)nc2)cn2ncc(C#N)c12] |
| 641 | 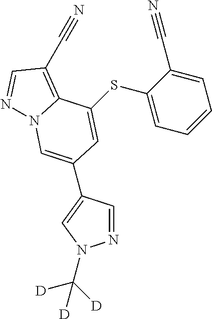 [SMILES: [2H]C([2H])([2H])n1cc(-c2cc(Sc3ccccc3C#N)c3c(C#N)cnn3c2)cn1] |
| 642 | 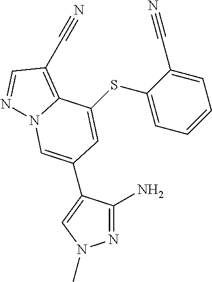 [SMILES: Cn1cc(-c2cc(Sc3ccccc3C#N)c3c(C#N)cnn3c2)c(N)n1] |
| 643 | 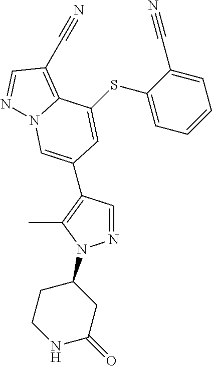 [SMILES: Cc1c(-c2cc(Sc3ccccc3C#N)c3c(C#N)cnn3c2)cnn1[C@@H]1CCNC(=O)C1] |
| 644 | 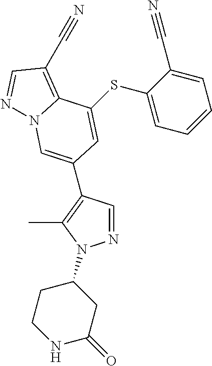 [SMILES: Cc1c(-c2cc(Sc3ccccc3C#N)c3c(C#N)cnn3c2)cnn1[C@H]1CCNC(=O)C1] |
| 645 | 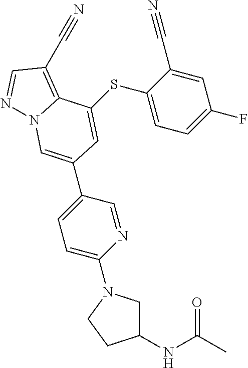 [SMILES: CC(=O)NC1CCN(c2ccc(-c3cc(Sc4ccc(F)cc4C#N)c4c(C#N)cnn4c3)cn2)C1] |
| 646 | 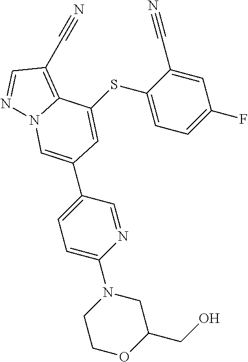 [SMILES: N#Cc1cc(F)ccc1Sc1cc(-c2ccc(N3CCOC(CO)C3)nc2)cn2ncc(C#N)c12] |
| 647 | 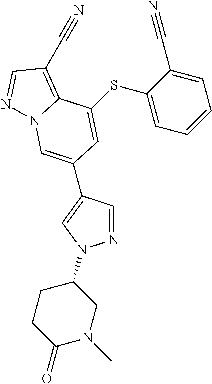 [SMILES: CN1C[C@@H](n2cc(-c3cc(Sc4ccccc4C#N)c4c(C#N)cnn4c3)cn2)CCC1=O] |
| 648 | 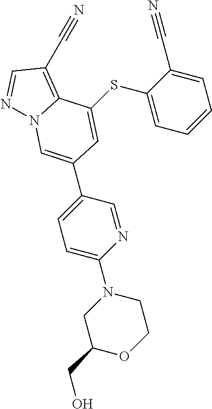 [SMILES: N#Cc1ccccc1Sc1cc(-c2ccc(N3CCO[C@@H](CO)C3)nc2)cn2ncc(C#N)c12] |
| 649 | 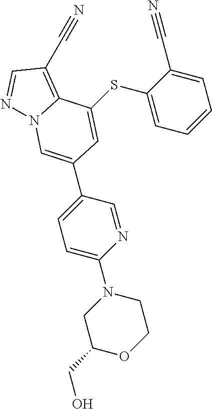 [SMILES: N#Cc1ccccc1Sc1cc(-c2ccc(N3CCO[C@H](CO)C3)nc2)cn2ncc(C#N)c12] |
| 650 | 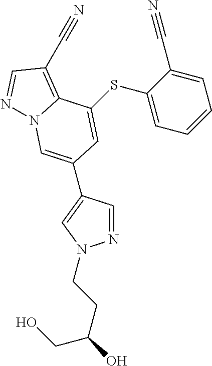 [SMILES: N#Cc1ccccc1Sc1cc(-c2cnn(CC[C@@H](O)CO)c2)cn2ncc(C#N)c12] |
| 651 | 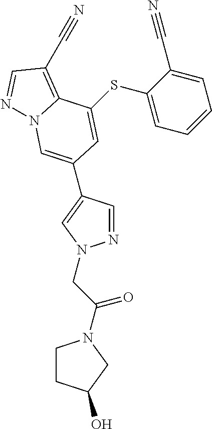 [SMILES: N#Cc1ccccc1Sc1cc(-c2cnn(CC(=O)N3CC[C@H](O)C3)c2)cn2ncc(C#N)c12] |
| 652 | 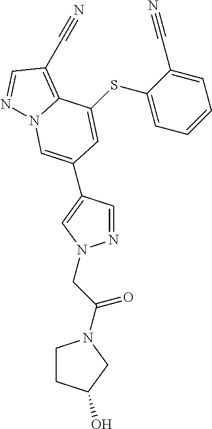 [SMILES: N#Cc1ccccc1Sc1cc(-c2cnn(CC(=O)N3CC[C@@H](O)C3)c2)cn2ncc(C#N)c12] |
| 653 | 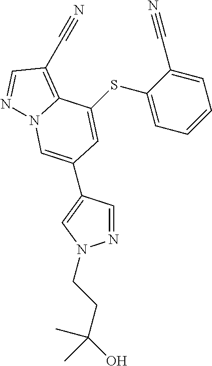 [SMILES: CC(C)(O)CCn1cc(-c2cc(Sc3ccccc3C#N)c3c(C#N)cnn3c2)cn1] |
| 654 | 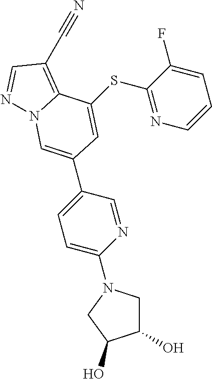 [SMILES: N#Cc1cnn2cc(-c3ccc(N4C[C@H](O)[C@@H](O)C4)nc3)cc(Sc3ncccc3F)c12] |
| 655 | 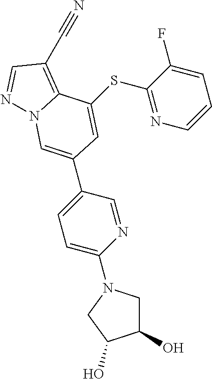 [SMILES: N#Cc1cnn2cc(-c3ccc(N4C[C@@H](O)[C@H](O)C4)nc3)cc(Sc3ncccc3F)c12] |
| 656 | 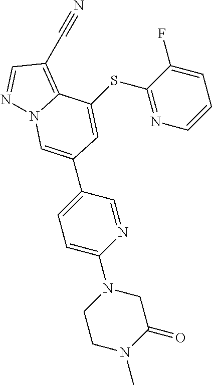 [SMILES: CN1CCN(c2ccc(-c3cc(Sc4ncccc4F)c4c(C#N)cnn4c3)cn2)CC1=O] |
| 657 | 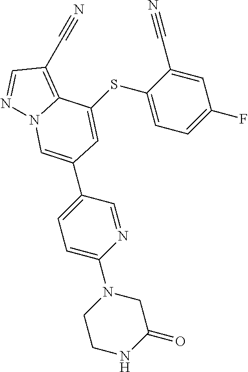 [SMILES: N#Cc1cc(F)ccc1Sc1cc(-c2ccc(N3CCNC(=O)C3)nc2)cn2ncc(C#N)c12] |
| 658 | 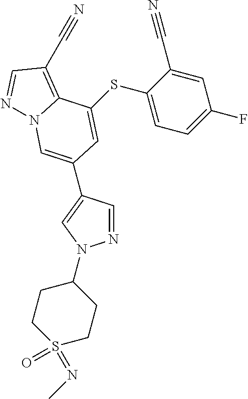 [SMILES: CN=S1(=O)CCC(n2cc(-c3cc(Sc4ccc(F)cc4C#N)c4c(C#N)cnn4c3)cn2)CC1] |
| 659 | 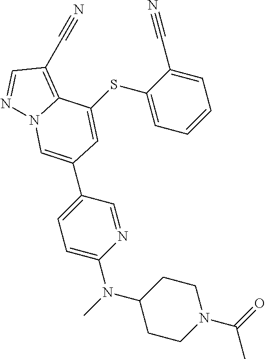 [SMILES: CC(=O)N1CCC(N(C)c2ccc(-c3cc(Sc4ccccc4C#N)c4c(C#N)cnn4c3)cn2)CC1] |
| 660 | 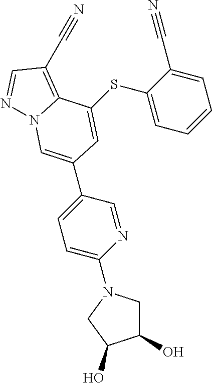 [SMILES: N#Cc1ccccc1Sc1cc(-c2ccc(N3C[C@@H](O)[C@@H](O)C3)nc2)cn2ncc(C#N)c12] |
| 661 | 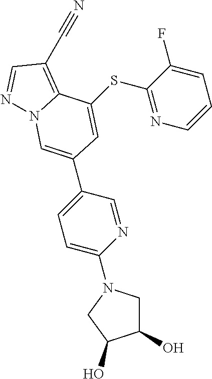 [SMILES: N#Cc1cnn2cc(-c3ccc(N4C[C@@H](O)[C@@H](O)C4)nc3)cc(Sc3ncccc3F)c12] |
| 662 | 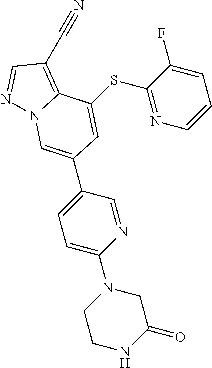 [SMILES: N#Cc1cnn2cc(-c3ccc(N4CCNC(=O)C4)nc3)cc(Sc3ncccc3F)c12] |
| 663 | 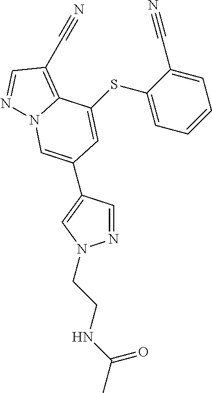 [SMILES: CC(=O)NCCn1cc(-c2cc(Sc3ccccc3C#N)c3c(C#N)cnn3c2)cn1] |
| 664 | 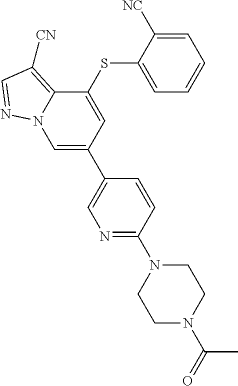 [SMILES: CC(=O)N1CCN(c2ccc(-c3cc(Sc4ccccc4C#N)c4c(C#N)cnn4c3)cn2)CC1] |
| 665 | 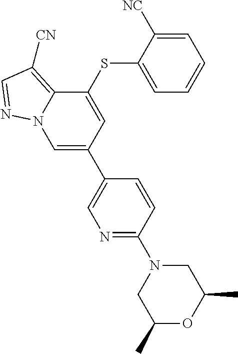 [SMILES: C[C@@H]1CN(c2ccc(-c3cc(Sc4ccccc4C#N)c4c(C#N)cnn4c3)cn2)C[C@H](C)O1] |
| 666 | 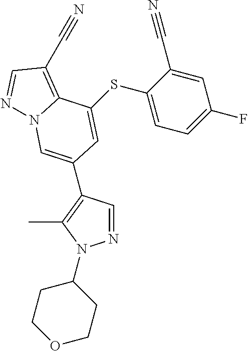 [SMILES: Cc1c(-c2cc(Sc3ccc(F)cc3C#N)c3c(C#N)cnn3c2)cnn1C1CCOCC1] |
| 667 | 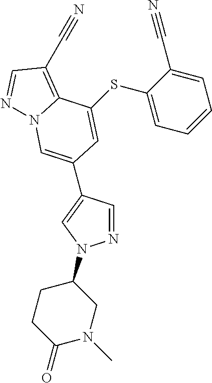 [SMILES: CN1C[C@H](n2cc(-c3cc(Sc4ccccc4C#N)c4c(C#N)cnn4c3)cn2)CCC1=O] |
| 668 | 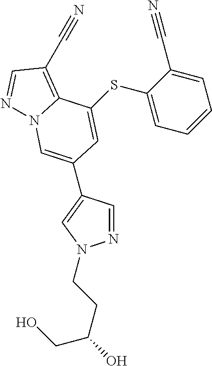 [SMILES: N#Cc1ccccc1Sc1cc(-c2cnn(CC[C@H](O)CO)c2)cn2ncc(C#N)c12] |
| 669 | 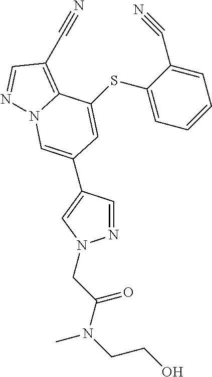 [SMILES: CN(CCO)C(=O)Cn1cc(-c2cc(Sc3ccccc3C#N)c3c(C#N)cnn3c2)cn1] |
| 670 | 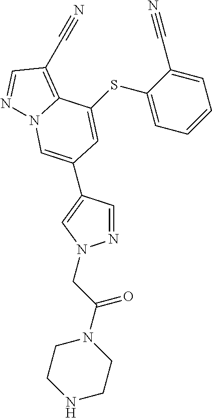 [SMILES: N#Cc1ccccc1Sc1cc(-c2cnn(CC(=O)N3CCNCC3)c2)cn2ncc(C#N)c12] |
| 671 | 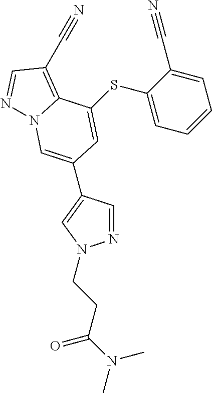 [SMILES: CN(C)C(=O)CCn1cc(-c2cc(Sc3ccccc3C#N)c3c(C#N)cnn3c2)cn1] |
| 672 | 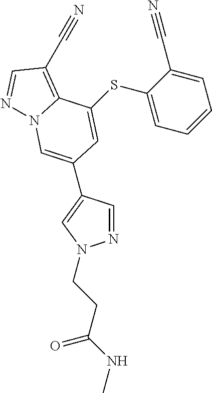 [SMILES: CNC(=O)CCn1cc(-c2cc(Sc3ccccc3C#N)c3c(C#N)cnn3c2)cn1] |
| 673 | 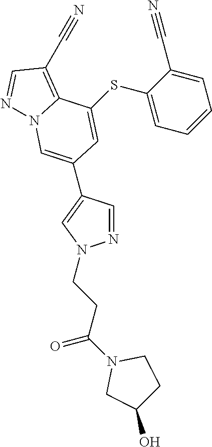 [SMILES: N#Cc1ccccc1Sc1cc(-c2cnn(CCC(=O)N3CC[C@@H](O)C3)c2)cn2ncc(C#N)c12] |
| 674 | 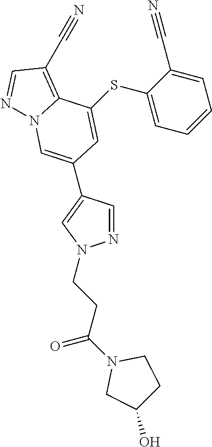 [SMILES: N#Cc1ccccc1Sc1cc(-c2cnn(CCC(=O)N3CC[C@H](O)C3)c2)cn2ncc(C#N)c12] |
| 675 | 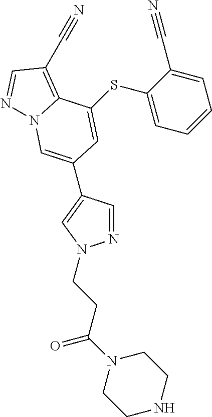 [SMILES: N#Cc1ccccc1Sc1cc(-c2cnn(CCC(=O)N3CCNCC3)c2)cn2ncc(C#N)c12] |
| 676 | 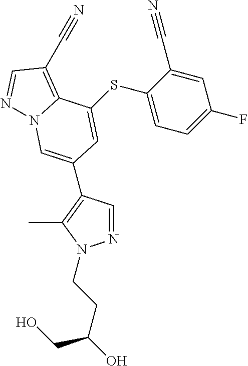 [SMILES: Cc1c(-c2cc(Sc3ccc(F)cc3C#N)c3c(C#N)cnn3c2)cnn1CC[C@@H](O)CO] |
| 677 | 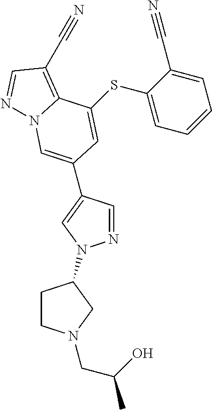 [SMILES: C[C@H](O)CN1CC[C@H](n2cc(-c3cc(Sc4ccccc4C#N)c4c(C#N)cnn4c3)cn2)C1] |
| 678 | 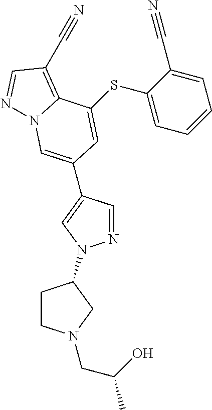 [SMILES: C[C@@H](O)CN1CC[C@H](n2cc(-c3cc(Sc4ccccc4C#N)c4c(C#N)cnn4c3)cn2)C1] |
| 679 | 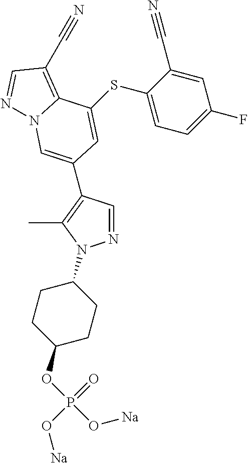 |
| 680 | 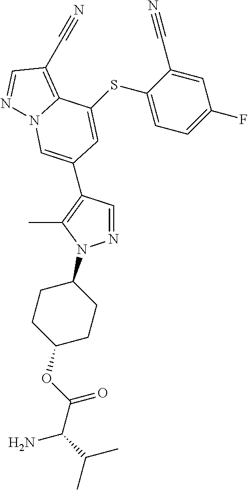 [SMILES: Cc1c(-c2cc(Sc3ccc(F)cc3C#N)c3c(C#N)cnn3c2)cnn1[C@H]1CC[C@H](OC(=O)[C@@H](N)C(C)C)CC1] |

As can be appreciated, the ionizable lipids described herein enable the development of new therapies for disease without the need for exotic chemistry or specialized reagents or manufacturing techniques.

### III Pharmaceutical compositions

Other embodiments are directed to pharmaceutical compositions. In an embodiment, the pharmaceutical composition comprises any one (or more) of the foregoing compounds and a pharmaceutically acceptable carrier. In some embodiments, the pharmaceutical composition is formulated for oral administration. In other embodiments, the pharmaceutical composition is formulated for injection. In still more embodiments, the pharmaceutical compositions comprise a compound as disclosed herein and an additional therapeutic agent (e.g., anticancer agent). Non-limiting examples of such additional therapeutic agents are described herein below.

Suitable routes of administration include, but are not limited to, oral, intravenous, rectal, aerosol, parenteral, ophthalmic, pulmonary, transmucosal, transdermal, vaginal, optical, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound as described herein is administered in a local rather than systemic manner, for example, via injection of the compound directly into an organ, often in a depot preparation or sustained release formulation. In specific embodiments, long acting formulations are administered by implantation (for example subcutaneously or intramuscularly) or by intramuscular injection. Furthermore, in other embodiments, the compound is delivered in a targeted drug delivery system, for example, in a liposome coated with and organ specific antibody. In such embodiments, the liposomes are targeted to and taken up selectively by the organ. In yet other embodiments, the compound as described herein is provided in the form of a rapid release formulation, in the form of an extended release formulation, or in the form of an intermediate release formulation. In yet other embodiments, the compound described herein is administered topically.

In treatment methods according to embodiments of the disclosure, an effective amount of at least one compound of Formula (I)-(IL) is administered to a subject suffering from or diagnosed as having such a disease, disorder, or medical condition. Effective amounts or doses may be ascertained by methods such as modeling, dose escalation studies or clinical trials, e.g., the mode or route of administration or drug delivery, the pharmacokinetics of the agent, the severity and course of the disease, disorder, or condition, the subject's previous or ongoing therapy, the subject's health status and response to drugs, and the judgment of the treating physician.

The compounds according to the disclosure are effective over a wide dosage range. For example, in the treatment of adult humans, dosages from about 0.001 to 0.1 mg, 0.01 to 0.1 mg, 0.5 to 5 mg, 0.5 to 10 mg, 0.01-10 mg, 0.1 to 10 mg, 10 to 5000 mg, 100 to 5000 mg, 1000 mg to 4000 mg per day, or 1000 to 3000 mg per day are examples of dosages that are used in some embodiments. The exact dosage will depend upon the route of administration, the form in which the compound is administered, the subject to be treated, the body weight of the subject to be treated, and the preference and experience of the attending physician.

In some embodiments, compounds of the disclosure are administered in a single dose. In an embodiment, the single dose is administered orally. In another embodiment, the single dose is administered topically. However, other routes are used as appropriate. In some embodiments, compounds of the disclosure are administered in multiple doses. In some embodiments, dosing is about once, twice, three times, four times, five times, six times, or more than six times per day. In other embodiments, dosing is about once a month, once every two weeks, once a week, or once every other day. In another embodiment compounds of the disclosure and another agent (e.g., anti-cancer agent) are administered together about once per day to about 6 times per day. In another embodiment the administration of compounds of the disclosure and an agent continues for less than about 7 days. In yet another embodiment the administration continues for more than about 6, 10, 14, 28 days, two months, six months, or one year. In some cases, continuous dosing is achieved and maintained as long as necessary.

Administration of compounds of the disclosure may continue as long as necessary. In some embodiments, compounds of the disclosure are administered for more than 1, 2, 3, 4, 5, 6, 7, 14, or 28 days. In some embodiments, compounds of the disclosure are administered for less than 28, 14, 7, 6, 5, 4, 3, 2, or 1 day. In some embodiments, compounds of the disclosure are administered chronically on an ongoing basis, e.g., for the treatment of chronic effects.

In some embodiments, the compounds of the disclosure are administered in individual dosage forms. It is known in the art that due to intersubject variability in compound pharmacokinetics, individualization of dosing regimen is necessary for optimal therapy.

In some embodiments, the compounds described herein are formulated into pharmaceutical compositions. In specific embodiments, pharmaceutical compositions are formulated in a conventional manner using one or more physiologically acceptable carriers comprising excipients and auxiliaries which facilitate processing of the disclosed compounds into preparations which can be used pharmaceutically. Proper formulation is dependent upon the route of administration chosen. Any pharmaceutically acceptable techniques, carriers, and excipients are used as suitable to formulate the pharmaceutical compositions described herein: Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Provided herein are pharmaceutical compositions comprising one or more compounds of Formula (I)-(IL), and a pharmaceutically acceptable carrier. Also provided herein are pharmaceutical compositions comprising one or more compounds selected from compounds of Formula (I)-(IL) and pharmaceutically acceptable diluent(s), excipient(s), and carrier(s). In certain embodiments, the compounds described are administered as pharmaceutical compositions in which one or more compounds selected from compounds of Formula (I)-(IL) are mixed with other active ingredients, as in combination therapy. Encompassed herein are all combinations of actives set forth in the combination therapies section below and throughout this disclosure. In specific embodiments, the pharmaceutical compositions include one or more compounds of Formula (I)-(IL).

A pharmaceutical composition, as used herein, refers to a mixture of one or more compounds selected from compounds of Formula (I)-(IL) with other chemical components, such as carriers, stabilizers, diluents, dispersing agents, suspending agents, thickening agents, and/or excipients. In certain embodiments, the pharmaceutical composition facilitates administration of the compound to an organism. In some embodiments, therapeutically effective amounts of one or more compounds selected from compounds of Formula (I)-(IL) provided herein are administered in a pharmaceutical composition to a mammal having a disease, disorder or medical condition to be treated. In specific embodiments, the mammal is a human. In certain embodiments, therapeutically effective amounts vary depending on the severity of the disease, the age and relative health of the subject, the potency of the compound used and other factors. The compounds described herein are used singly or in combination with one or more therapeutic agents as components of mixtures.

In one embodiment, one or more compounds selected from compounds of Formula (I)-(IF) are formulated in aqueous solutions. In specific embodiments, the aqueous solution is selected from, by way of example only, a physiologically compatible buffer, such as Hank's solution, Ringer's solution, or physiological saline buffer. In other embodiments, one or more compounds selected from compounds of Formula (I)-(IL) are formulated for transmucosal administration. In specific embodiments, transmucosal formulations include penetrants that are appropriate to the barrier to be permeated. In still other embodiments wherein the compounds described herein are formulated for other parenteral injections, appropriate formulations include aqueous or non-aqueous solutions. In specific embodiments, such solutions include physiologically compatible buffers and/or excipients.

In another embodiment, compounds described herein are formulated for oral administration. Compounds described herein are formulated by combining the active compounds with, e.g., pharmaceutically acceptable carriers or excipients. In various embodiments, the compounds described herein are formulated in oral dosage forms that include, by way of example only, tablets, powders, pills, dragees, capsules, liquids, gels, syrups, elixirs, slurries, suspensions and the like.

In certain embodiments, pharmaceutical preparations for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In specific embodiments, disintegrating agents are optionally added. Disintegrating agents include, by way of example only, cross linked croscarmellose sodium, polyvinylpyrrolidone, agar, or alginic acid or a salt thereof such as sodium alginate.

In one embodiment, the oral dosage forms, such as a pill, capsule or tablet, comprises one or more suitable layers or coatings. In specific embodiments, concentrated sugar solutions are used for coating the dosage form. The sugar solutions, optionally contain additional components, such as by way of example only, gum arabic, talc, polyvinylpyrrolidone, carbopol gel, polyethylene glycol, and/or titanium dioxide, lacquer solutions, and suitable organic solvents or solvent mixtures. Dyestuffs and/or pigments are also optionally added to the coatings for identification purposes. Additionally, the dyestuffs and/or pigments are optionally utilized to characterize different combinations of active compound doses.

In certain embodiments, therapeutically effective amounts of at least one of the compounds described herein are formulated into other oral dosage forms. Oral dosage forms include push fit capsules made of gelatin, as well as soft, sealed capsules made of gelatin and a plasticizer, such as glycerol or sorbitol. In specific embodiments, push fit capsules contain the active ingredients in admixture with one or more filler. Fillers include, by way of example only, lactose, binders such as starches, and/or lubricants such as talc or magnesium stearate and, optionally, stabilizers. In other embodiments, soft capsules, contain one or more active compound that is dissolved or suspended in a suitable liquid. Suitable liquids include, by way of example only, one or more fatty oil, liquid paraffin, or liquid polyethylene glycol. In addition, stabilizers are optionally added.

In still other embodiments, the compounds described herein are formulated for parental injection, including formulations suitable for bolus injection or continuous infusion. In specific embodiments, formulations for injection are presented in unit dosage form (e.g., in ampoules) or in multi dose containers. Preservatives are, optionally, added to the injection formulations. In still other embodiments, the pharmaceutical compositions are formulated in a form suitable for parenteral injection as sterile suspensions, solutions or emulsions in oily or aqueous vehicles. Parenteral injection formulations optionally contain formulatory agents such as suspending, stabilizing and/or dispersing agents. In specific embodiments, pharmaceutical formulations for parenteral administration include aqueous solutions of the active compounds in water soluble form. In additional embodiments, suspensions of one or more compounds selected from compounds of Formula (I)-(IL) are prepared as appropriate oily injection suspensions. Suitable lipophilic solvents or vehicles for use in the pharmaceutical compositions described herein include, by way of example only, fatty oils such as sesame oil, or synthetic fatty acid esters, such as ethyl oleate or triglycerides, or liposomes. In certain specific embodiments, aqueous injection suspensions contain substances which increase the viscosity of the suspension, such as sodium carboxymethyl cellulose, sorbitol, or dextran. Optionally, the suspension contains suitable stabilizers or agents which increase the solubility of the compounds to allow for the preparation of highly concentrated solutions. Alternatively, in other embodiments, the active ingredient is in powder form for constitution with a suitable vehicle, e.g., sterile pyrogen-free water, before use.

Pharmaceutical compositions include at least one pharmaceutically acceptable carrier, diluent or excipient, and one or more compounds selected from compounds of Formula (I)-(IL) as an active ingredient. The active ingredient is in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. All tautomers of the compounds described herein are included within the scope of the compounds presented herein. Additionally, the compounds described herein encompass unsolvated as well as solvated forms with pharmaceutically acceptable solvents such as water, ethanol, and the like. The solvated forms of the compounds presented herein are also considered to be disclosed herein. In addition, the pharmaceutical compositions optionally include other medicinal or pharmaceutical agents, carriers, adjuvants, such as preserving, stabilizing, wetting or emulsifying agents, solution promoters, salts for regulating the osmotic pressure, buffers, and/or other therapeutically valuable substances.

Methods for the preparation of compositions comprising the compounds described herein include formulating the compound(s) with one or more inert, pharmaceutically acceptable excipients or carriers to form a solid, semi-solid or liquid composition. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, ointments, suspensions and creams. The form of the pharmaceutical compositions described herein include liquid solutions or suspensions, solid forms suitable for solution or suspension in a liquid prior to use, or as emulsions. These compositions also optionally contain minor amounts of nontoxic, auxiliary substances, such as wetting or emulsifying agents, pH buffering agents, and so forth.

In some embodiments, pharmaceutical compositions comprising one or more compounds selected from compounds of Formula (I)-(IL) illustratively takes the form of a liquid where the agents are present in solution, in suspension or both. Typically when the composition is administered as a suspension, a first portion of the agent is present in solution and a second portion of the agent is present in particulate form, in suspension in a liquid matrix. In some embodiments, a liquid composition includes a gel formulation. In other embodiments, the liquid composition is aqueous.

In certain embodiments, aqueous suspensions contain one or more polymers as suspending agents. Polymers include water-soluble polymers such as cellulosic polymers, e.g., hydroxypropyl methylcellulose, and water-insoluble polymers such as cross-linked carboxyl-containing polymers. Certain pharmaceutical compositions described herein comprise a mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

Pharmaceutical compositions also, optionally, include solubilizing agents to aid in the solubility of one or more compounds selected from compounds of Formula (I)-(IL). The term “solubilizing agent” generally includes agents that result in formation of a micellar solution or a true solution of the agent. Certain acceptable nonionic surfactants, for example polysorbate 80, are useful as solubilizing agents, as can ophthalmically acceptable glycols, polyglycols, e.g., polyethylene glycol 400, and glycol ethers.

Furthermore, pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Compositions also, optionally, include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Compositions may include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Compositions may include one or more antioxidants to enhance chemical stability where required. Suitable antioxidants include, by way of example only, ascorbic acid and sodium metabisulfite.

In certain embodiments, aqueous suspension compositions are packaged in single-dose non-reclosable containers. Alternatively, multiple-dose reclosable containers are used, in which case it is typical to include a preservative in the composition.

In alternative embodiments, other delivery systems for hydrophobic pharmaceutical compounds are employed. Liposomes and emulsions are examples of delivery vehicles or carriers useful herein. In certain embodiments, organic solvents such as N-methylpyrrolidone are also employed. In additional embodiments, the compounds described herein are delivered using a sustained release system, such as semipermeable matrices of solid hydrophobic polymers containing the therapeutic agent. Various sustained release materials are useful herein. In some embodiments, sustained release capsules release the compounds for a few weeks up to over 100 days. Depending on the chemical nature and the biological stability of the therapeutic reagent, additional strategies for protein stabilization are employed.

In certain embodiments, the formulations described herein comprise one or more antioxidants, metal chelating agents, thiol containing compounds and/or other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (c) about 0.01% to about 2% w/v ascorbic acid, (f) 0.003% to about 0.02% w/v polysorbate 80, (g) 0.001% to about 0.05% w/v, polysorbate 20, (h) arginine, (i) heparin, (j) dextran sulfate, (k) cyclodextrins, (l) pontosan polysulfate and other heparinoids, (m) divalent cations such as magnesium and zinc; or (n) combinations thereof.

In some embodiments, the concentration of one or more compounds selected from compounds of Formula (I)-(IL) provided in the pharmaceutical compositions is greater than 90%, 80%, 75%, 70%, 65%, 60%, 55%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25%, 7%, 6.75%, 6.50%, 6.25% 6%, 5.75%, 5.50%, 5.25%, 5%, 4.75%, 4.50%, 4.25%, 4%, 3.75%, 3.50%, 3.25%, 3%, 2.75%, 2.50%, 2.25%, 2%, 1.75%, 1.50%, 1.25%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v. In another embodiment, the amount of a compound selected from compounds of Formula (I)-(IL) in the pharmaceutical compositions is an amount between about any two of the values recited in the preceding sentence, for example, between about 2-70 w/w %, 3.5-80 w/w %, 1-30 w/w %, etc.

In some embodiments, the concentration of one or more compounds selected from compounds of Formula (I)-(IL) provided in the pharmaceutical compositions of the present disclosure is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v.

In some embodiments, the amount the one or more compounds selected from compounds of Formula (I)-(IL) provided in the pharmaceutical compositions of the present disclosure is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of the one or more compounds selected from compounds of Formula (I)-(IL) provided in the pharmaceutical compositions of the present disclosure is in the range of 0.0001-10 g, 0.0005-9 g, 0.001-8 g, 0.005-7 g, 0.01-6 g, 0.05-5 g, 0.1-4 g, 0.5-4 g, or 1-3 g.

Packaging materials for use in packaging pharmaceutical compositions described herein include those found in, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. For example, the container(s) includes one or more compounds described herein, optionally in a composition or in combination with another agent as disclosed herein. The container(s) optionally have a sterile access port (for example the container is an intravenous solution bag or a vial having a stopper pierceable by a hypodermic injection needle). Such kits optionally comprise a compound with an identifying description or label or instructions relating to its use in the methods described herein.

For example, a kit typically includes one or more additional containers, each with one or more of various materials (such as reagents, optionally in concentrated form, and/or devices) desirable from a commercial and user standpoint for use of a compound described herein. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included. A label is optionally on or associated with the container. For example, a label is on a container when letters, numbers or other characters forming the label are attached, molded or etched into the container itself, a label is associated with a container when it is present within a receptacle or carrier that also holds the container, e.g., as a package insert. In addition, a label is used to indicate that the contents are to be used for a specific therapeutic application. In addition, the label indicates directions for use of the contents, such as in the methods described herein. In certain embodiments, the pharmaceutical compositions are presented in a pack or dispenser device which contains one or more unit dosage forms containing a compound provided herein. The pack for example contains metal or plastic foil, such as a blister pack. Or, the pack or dispenser device is accompanied by instructions for administration. Or, the pack or dispenser is accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In some embodiments, compositions containing a compound provided herein formulated in a compatible pharmaceutical carrier are prepared, placed in an appropriate container, and labeled for treatment of an indicated condition.

As mentioned above, the compounds and compositions of the disclosure will find utility in a broad range of diseases and conditions mediated by protein kinases, including diseases and conditions mediated by kinase. Such diseases may include by way of example and not limitation, cancers such as lung cancer, NSCLC (non small cell lung cancer), oat-cell cancer, bone cancer, pancreatic cancer, skin cancer, dermatofibrosarcoma protuberans, cancer of the head and neck, cutaneous or intraocular melanoma, uterine cancer, ovarian cancer, colon-rectal cancer, cancer of the anal region, stomach cancer, colon cancer, breast cancer, gynecologic tumors (e.g., uterine sarcomas, carcinoma of the fallopian tubes, carcinoma of the endometrium, carcinoma of the cervix, carcinoma of the vagina or carcinoma of the vulva), Hodgkin's Disease, hepatocellular cancer, cancer of the esophagus, cancer of the small intestine, cancer of the endocrine system (e.g., cancer of the thyroid, pancreas, parathyroid or adrenal glands), sarcomas of soft tissues, cancer of the urethra, cancer of the penis, prostate cancer (particularly hormone-refractory), chronic or acute leukemia, solid tumors of childhood, hypercosinophilia, lymphocytic lymphomas, cancer of the bladder, cancer of the kidney or ureter (e.g., renal cell carcinoma, carcinoma of the renal pelvis), pediatric malignancy, neoplasms of the central nervous system (e.g., primary CNS lymphoma, spinal axis tumors, medulloblastoma, brain stem gliomas or pituitary adenomas), Barrett's esophagus (pre-malignant syndrome), neoplastic cutaneous disease, psoriasis, mycoses fungoides, and benign prostatic hypertrophy, diabetes related diseases such as diabetic retinopathy, retinal ischemia, and retinal neovascularization, hepatic cirrhosis, angiogenesis, cardiovascular disease such as atherosclerosis, immunological disease such as autoimmune disease and renal disease.

In some embodiments, a pharmaceutical composition has a compound described above and a pharmaceutically acceptable carrier including, for example, any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals. In some embodiments, the compound of the pharmaceutical composition for use in treating a disease associated with mutations in fibroblast growth factor receptor 2 (FGFR2) or fibroblast growth factor receptor 3 (FGFR3) is provided.

In some embodiments, a method of treating a disease associated with mutations in FGFR2 or FGFR3, comprising: administering the compound or the pharmaceutical composition to a subject in need thereof. In some embodiments, the subject is an animal. In some embodiments, the subject is a human. In some embodiments, the disease associated with mutations in FGFR2 is a cancer or craniosynostoic syndrome. In some certain embodiments, the cancer is intrahepatic cholangiocarcinoma, hepatocellular carcinoma, breast cancer, prostate cancer, lung squamous cell carcinoma, thyroid cancer, gastric cancer, ovarian cancer, endometrial cancer, non-small cell lung cancer, urothelial cancers, colorectal cancer, colon cancer, metastatic cholangiocarcinoma, cholangiocarcinoma, osteosarcoma, gastroesophageal junction adenocarcinoma, biliary tract cancer, anaplastic thyroid carcinoma, ganglioglioma, pancreatic intraductal tubulopapillary neoplasm, gallbladder carcinoma, renal cell carcinoma, myxoid lipocarcinoma, triple negative breast cancer, or rectal cancer. In some certain embodiments, the craniosynostoic syndrome is craniosynostosis, bent bone dysplasia, crouzon syndrome, apert syndrome, pfeiffer syndrome, antley-bixler, beare-stevenson syndrome, jackson-weiss syndrome, or seathre-chotzen-like syndromes. In some embodiments, the disease associated with mutations in FGFR3 is systemic sclerosis, fibrosis, pulmonary fibrosis, achondroplasia, thanatophoric dysplasia, severe achondroplasia with developmental delay and acanthosis nigricans (SADDAN syndrome), muenke syndrome, FGFR3 associated cancer, hypochondroplasia, FGFR3 related craniosynostosis, LADD syndrome, or Alzheimer disease.

### IV Methods of Preparation

Preparation methods for the above compounds and compositions are described herein below and/or known in the art.

It will be appreciated by those skilled in the art that in the process described herein the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include —C(O)—R″ (where R″ is alkyl, aryl or arylalkyl), p-methoxybenzyl, trityl and the like. Suitable protecting groups for carboxylic acid include alkyl, aryl or arylalkyl esters. Protecting groups may be added or removed in accordance with standard techniques, which are known to one skilled in the art and as described herein. The use of protecting groups is described in detail in Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin or a 2-chlorotrityl-chloride resin.

It will also be appreciated by those skilled in the art, although such protected derivatives of compounds of this invention may not possess pharmacological activity as such, they may be administered to a mammal and thereafter metabolized in the body to form compounds of the invention which are pharmacologically active. Such derivatives may therefore be described as “prodrugs”. All prodrugs of compounds of this invention are included within the scope of the invention.

Furthermore, all compounds of the invention which exist in free base or acid form can be converted to their pharmaceutically acceptable salts by treatment with the appropriate inorganic or organic base or acid by methods known to one skilled in the art. Salts of the compounds of the invention can be converted to their free base or acid form by standard techniques.

Syntheses for the compounds of Formula (I) are described below.

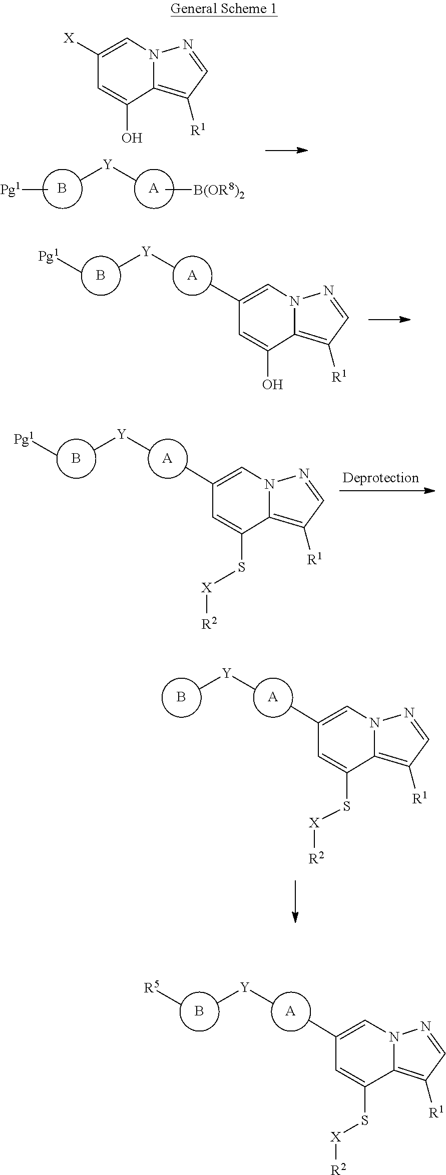

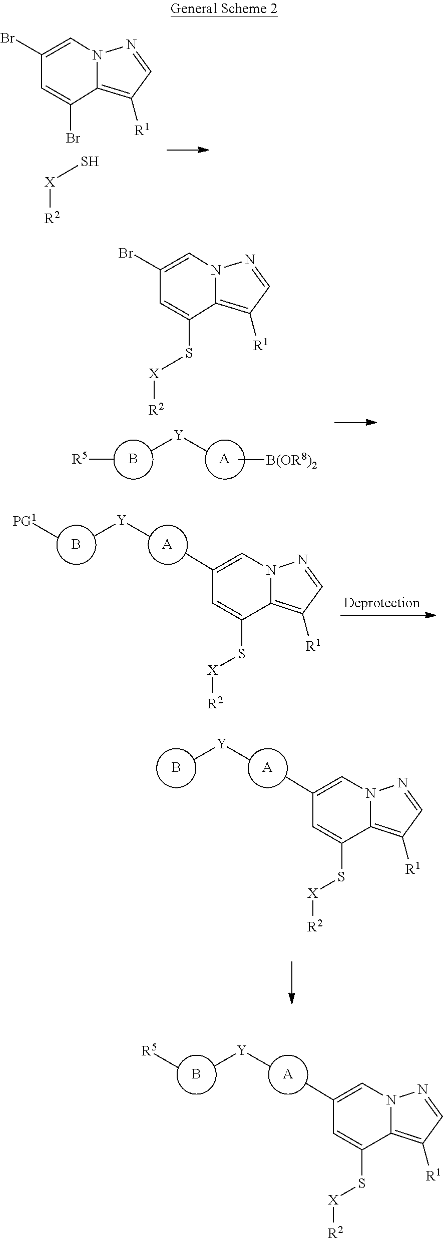

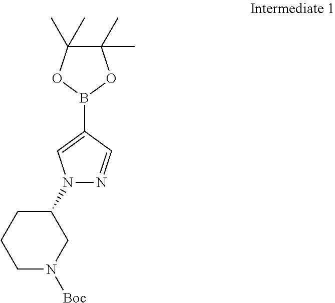 [SMILES: CC(C)(C)OC(=O)N1CCC[C@H](n2cc(B3OC(C)(C)C(C)(C)O3)cn2)C1]

### tert-butyl (3S)-3-[4-(4,4,5,5-tetramethyl-1,3,2-dioxaborolan-2-yl)pyrazol-1-yl]piperidine-1-carboxylate

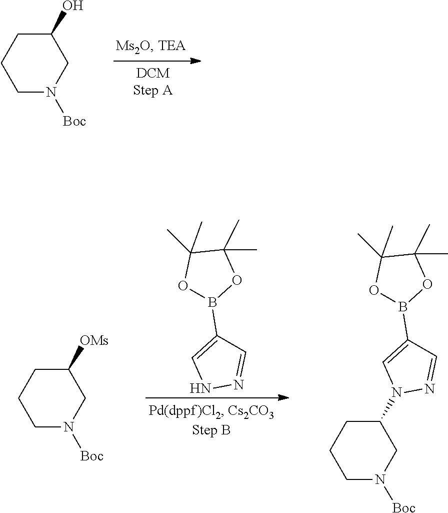

### Step A. tert-butyl 3-methylsulfonyloxypiperidine-1-carboxylate

To a 40 mL vial flask equipped with a magnetic stir bar was added tert-butyl (3R)-3-hydroxypiperidine-1-carboxylate (0.50 g, 2.5 mmol) followed by the addition of DCM (12 mL). TRIETHYLAMINE (0.69 mL, 5.0 mmol) was added into the mixture. The solution was cooled to 0° C. and methanesulfonic anhydride (0.86 g, 5.0 mmol) was added. The mixture was warmed to 25° C. and stirred for 1 h. Water (10 mL) was added to the reaction and the mixture was transferred to a separatory funnel, the aqueous layer was extracted with ethyl acetate (10 mL×3). The combined organic layers were washed with brine (10 mL), dried over anhydrous sodium sulfate, filtered, and concentrated under reduced pressure affording tert-butyl 3-methylsulfonyloxypiperidine-1-carboxylate (0.90 g, 99% yield) as yellow solid used into the next step without further purification.

### Step B. tert-butyl (3S)-3-[4-(4,4,5,5-tetramethyl-1,3,2-dioxaborolan-2-yl)pyrazol-1-yl]piperidine-1-carboxylate

To a 40 mL vial flask equipped with a magnetic stir bar and a reflux condenser was added tert-butyl 3-methylsulfonyloxypiperidine-1-carboxylate (0.65 g, 2.3 mmol) and 4-(4,4,5,5-tetramethyl-1,3,2-dioxaborolan-2-yl)-1H-pyrazole (0.45 g, 2.3 mmol) followed by the addition DMF (12 mL) and Cs₂CO₃ (1.5 g, 4.7 mmol) was added to the mixture. The mixture was heated to 90° C. and stirred for 12 h. Water (10 mL) was added to the reaction and the mixture was transferred to a separatory funnel and the aqueous layer was extracted with ethyl acetate (10 mL×3). The combined organic layer was washed with brine (10 mL), dried over anhydrous sodium sulfate, filtered, concentrated under reduced and purified by silica gel column chromatography (petroleum ether/ethyl acetate from 50/1 to 30/1) to give the desired product tert-butyl (3S)-3-[4-(4,4,5,5-tetramethyl-1,3,2-dioxaborolan-2-yl)pyrazol-1-yl]piperidine-1-carboxylate (0.80 g, 76% yield) as a yellow oil. LCMS (MM-ES+APCI, Pos): m/z 378.1 (M+H).

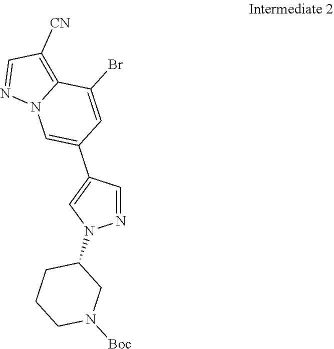 [SMILES: CC(C)(C)OC(=O)N1CCC[C@H](n2cc(-c3cc(Br)c4c(C#N)cnn4c3)cn2)C1]

### tert-butyl (3S)-3-[4-(4-bromo-3-cyano-pyrazolo[1,5-a]pyridin-6-yl)pyrazol-1-yl]piperidine-1-carboxylate

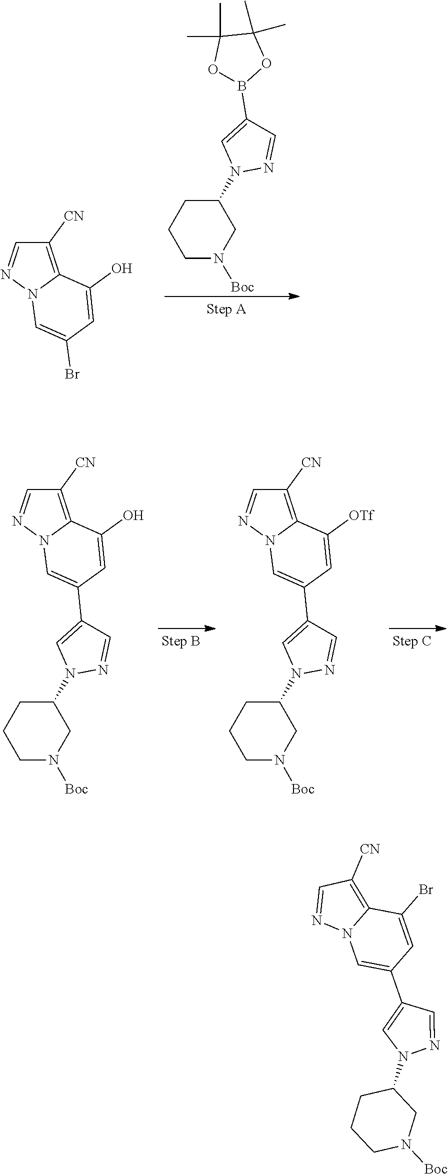 [SMILES: N#Cc1cnn2cc(Br)cc(O)c12 ; CC(C)(C)OC(=O)N1CCC[C@H](n2cc(B3OC(C)(C)C(C)(C)O3)cn2)C1 ; CC(C)(C)OC(=O)N1CCC[C@H](n2cc(-c3cc(O)c4c(C#N)cnn4c3)cn2)C1 ; CC(C)(C)OC(=O)N1CCC[C@H](n2cc(-c3cc(OS(=O)(=O)C(F)(F)F)c4c(C#N)cnn4c3)cn2)C1 ; CC(C)(C)OC(=O)N1CCC[C@H](n2cc(-c3cc(Br)c4c(C#N)cnn4c3)cn2)C1]

### Step A. tert-butyl (3S)-3-[4-(3-cyano-4-hydroxy-pyrazolo[1,5-a]pyridin-6-yl)pyrazol-1-yl]piperidine-1-carboxylate

To a 250 mL round-bottom flask equipped with a magnetic stir bar and a reflux condenser was added 6-bromo-4-hydroxy-pyrazolo[1,5-a]pyridine-3-carbonitrile (6.0 g, 25.2 mmol) and tert-butyl (3S)-3-[4-(4,4,5,5-tetramethyl-1,3,2-dioxaborolan-2-yl)pyrazol-1-yl]piperidine-1-carboxylate (12 g, 30 mmol) followed by the addition of dioxane (60 mL) and H₂O (12 mL). Pd₂(dba)₃ (0.46 mg, 2.5 mmol), XPhos (0.60 g, 1.3 mmol) and K₂CO₃ (7.0 g, 50 mmol) were added. The flask was evacuated and backfilled with nitrogen three times and the reaction was stirred at 90° C. under an atmosphere of nitrogen for 1 h. Water (100 mL) was added to the reaction and the mixture was transferred to a separatory funnel. The aqueous layer was extracted with ethyl acetate (50 mL×3). The combined organic layers were washed with brine (100 mL), dried over anhydrous sodium sulfate, filtered, and concentrated under reduced pressure to a give a brown solid. The solid was purified by silica gel column chromatography (30-100% ethyl acetate/petroleum ether) to give tert-butyl (3S)-3-[4-(3-cyano-4-hydroxy-pyrazolo[1,5-a]pyridin-6-yl)pyrazol-1-yl]piperidine-1-carboxylate (8.5 g, 79% yield) as a brown solid. LCMS (MM-ES+APCI, Pos): m/z 409.1 (M+H).

### Step B. tert-butyl (3S)-3-[4-[3-cyano-4-(trifluoromethylsulfonyloxy)pyrazolo[1,5-a]pyridin-6-yl]pyrazol-1-yl]piperidine-1-carboxylate

To a 500 mL round-bottom flask equipped with a magnetic stir bar was added tert-butyl (3S)-3-[4-(3-cyano-4-hydroxy-pyrazolo[1,5-a]pyridin-6-yl)pyrazol-1-yl]piperidine-1-carboxylate (10 g, 24 mmol) and 1,1,1-trifluoro-N-phenyl-N-(trifluoromethylsulfonyl)methanesulfonamide (18 g, 49 mmol) followed by the addition of DMF (150 mL). DIEA (8.5 mL, 49 mmol) was added and the mixture stirred at 25° C. for 2 h. Water (100 mL) was added to the reaction and the mixture was transferred to a separatory funnel, and the aqueous layer was extracted with ethyl acetate (50 mL×3). The combined organic layers were washed with brine (100 mL), dried over anhydrous sodium sulfate, filtered, concentrated under reduced pressure and purified by trituration in ethyl acetate (25 mL) to give tert-butyl (3S)-3-[4-[3-cyano-4-(trifluoromethylsulfonyloxy)pyrazolo[1,5-a]pyridin-6-yl]pyrazol-1-yl]piperidine-1-carboxylate (9.3 g, 87% yield) as a white solid. LCMS (MM-ES+APCI, Pos): m/z 541.3 (M+H).

### Step C. tert-butyl (3S)-3-[4-(4-bromo-3-cyano-pyrazolo[1,5-a]pyridin-6-yl)pyrazol-1-yl]piperidine-1-carboxylate

To a 40 mL vial equipped with a magnetic stir bar was added tert-butyl (3S)-3-[4-[3-cyano-4-(trifluoromethylsulfonyloxy)pyrazolo[1,5-a]pyridin-6-yl]pyrazol-1-yl]piperidine-1-carboxylate (1.6 g, 3.0 mmol) and LiBr (0.39 g, 4.4 mmol) followed by the addition of NMP (20 mL). Tris(acetonitrile)pentamethylcyclopentadienyl Ruthenium(II) Trifluoromethanesulfonate (0.10 g, 0.19 mmol) was added, the vial purged with nitrogen and the mixture stirred at 90° C. under an atmosphere of nitrogen for 1 h. The reaction was diluted with water (100 mL) and transferred to a separatory funnel. The aqueous layer was extracted with ethyl acetate (100 mL×3). The combined organic layers were washed with brine (100 mL), dried over anhydrous sodium sulfate, filtered, concentrated under reduced and purified by silica gel column chromatography (30-60% ethyl acetate/petroleum ether) to a brown solid. The solid was purified by trituration with MTBE (30 mL) to give tert-butyl (3S)-3-[4-(4-bromo-3-cyano-pyrazolo[1,5-a]pyridin-6-yl)pyrazol-1-yl]piperidine-1-carboxylate (0.32 g, 63% yield LCMS (MM-ES+APCI, Pos): m/z 473.2 (M+H).

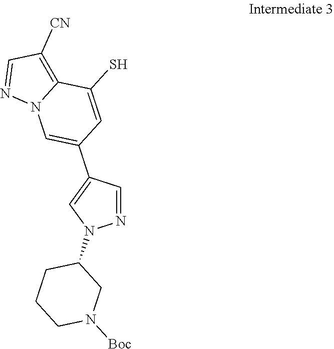 [SMILES: CC(C)(C)OC(=O)N1CCC[C@H](n2cc(-c3cc(S)c4c(C#N)cnn4c3)cn2)C1]

### tert-butyl (3S)-3-[4-(3-cyano-4-sulfanyl-pyrazolo[1,5-a]pyridin-6-yl)pyrazol-1-yl]piperidine-1-carboxylate

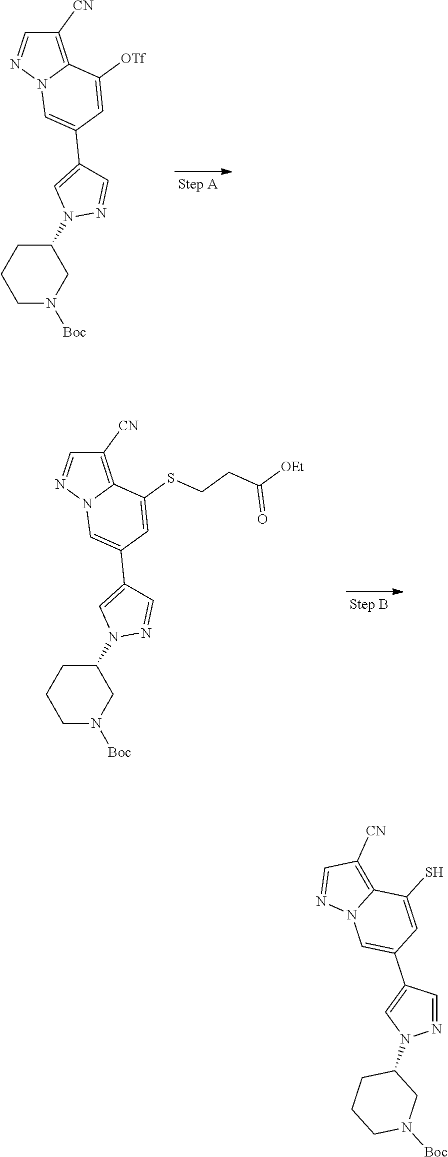 [SMILES: CC(C)(C)OC(=O)N1CCC[C@H](n2cc(-c3cc(OS(=O)(=O)C(F)(F)F)c4c(C#N)cnn4c3)cn2)C1 ; CCOC(=O)CCSc1cc(-c2cnn([C@H]3CCCN(C(=O)OC(C)(C)C)C3)c2)cn2ncc(C#N)c12 ; CC(C)(C)OC(=O)N1CCC[C@H](n2cc(-c3cc(S)c4c(C#N)cnn4c3)cn2)C1]

### Step A. tert-butyl (3S)-3-[4-[3-cyano-4-(3-ethoxy-3-oxo-propyl)sulfanyl-pyrazolo[1,5-a]pyridin-6-yl]pyrazol-1-yl]piperidine-1-carboxylate

To a 40 mL vial equipped with a magnetic stir bar was added tert-butyl (3S)-3-[4-[3-cyano-4-(trifluoromethylsulfonyloxy)pyrazolo[1,5-a]pyridin-6-yl]pyrazol-1-yl]piperidine-1-carboxylate (0.75 g, 1.3 mmol) followed by the addition of toluene (10 mL). Xantphos (74 mg, 0.13 mmol), Pd(OAc)₂ (33 mg, 0.13 mmol), K₃PO₄ (0.54 g, 2.5 mmol) and ethyl 3-sulfanylpropanoate (0.26 g, 1.9 mmol) were added into the mixture at 25° C. The vial was backfilled with nitrogen and stirred at 80° C. under an atmosphere of nitrogen for 16 h. Water (25 mL) was added to the reaction and the mixture was transferred to a separatory funnel. the aqueous layer was extracted with ethyl acetate (25 mL×3). The combined organic layers were washed with brine (80 mL), dried over anhydrous sodium sulfate, filtered, concentrated under reduced pressure and purified by silica gel column chromatography (40-50% ethyl acetate/petroleum ether) to give tert-butyl (3S)-3-[4-[3-cyano-4-(3-ethoxy-3-oxo-propyl)sulfanyl-pyrazolo[1,5-a]pyridin-6-yl]pyrazol-1-yl]piperidine-1-carboxylate (1.3 g, 93% yield) as a yellow gum. LCMS (MM-ES+APCI, Pos): m/z 525.4 (M+H).

### Step B. Tert-butyl (3S)-3-[4-(3-cyano-4-sulfanyl-pyrazolo[1,5-a]pyridin-6-yl)pyrazol-1-yl]piperidine-1-carboxylate

To a 40 mL vial equipped with a magnetic stir bar was added tert-butyl (3S)-3-[4-[3-cyano-4-(3-ethoxy-3-oxo-propyl)sulfanylpyrazolo[1,5-a]pyridin-6-yl]pyrazol-1-yl]piperidine-1-carboxylate (0.20 g, 0.36 mmol) followed by the addition of DMF (4 mL). The solution was cooled to 0° C. and treated with t-BuOK (1 M, 0.47 mL, 0.47 mmol) dropwise. The mixture was stirred at 0° C. for 1 h. The mixture was concentrated under reduced pressure affording tert-butyl (3S)-3-[4-(3-cyano-4-sulfanyl-pyrazolo[1,5-a]pyridin-6-yl)pyrazol-1-yl]piperidine-1-carboxylate (0.18 g, crude) which was used in the next step without further purification.

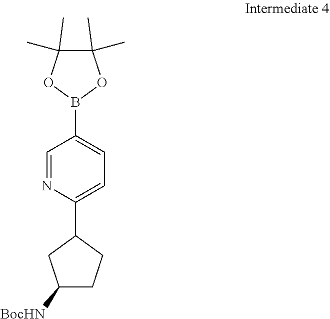

### tert-butyl N-[(3R)-1-[5-(4,4,5,5-tetramethyl-1,3,2-dioxaborolan-2-yl)-2-pyridyl] pyrrolidin-3-yl]carbamate

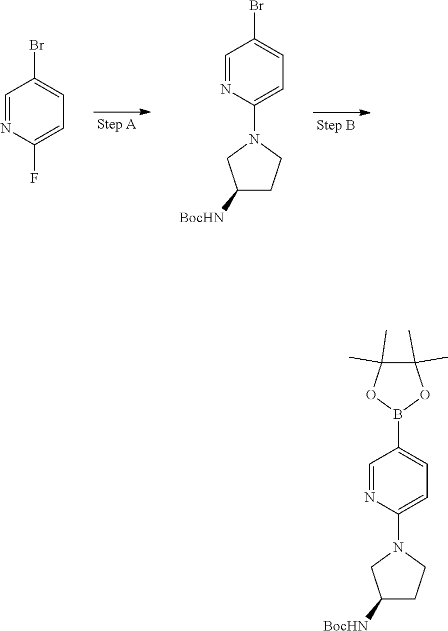

### Step A. tert-butyl N-[(3R)-1-(5-bromo-2-pyridyl)pyrrolidin-3-yl]carbamate

To a 40 mL vial equipped with a magnetic stir bar was added tert-butyl N-[(3R)-pyrrolidin-3-yl]carbamate (0.35 g, 1.9 mmol) in DMSO (7 mL). DIEA (1.3 mL, 7.6 mmol) and 5-bromo-2-fluoro-pyridine (0.41 g, 2.3 mmol) were added into the mixture. The vial was purged with nitrogen and heated to 90° C. for 1 h. Water (50 mL) was added to the reaction and the mixture was transferred to a separatory funnel. The aqueous layer was extracted with ethyl acetate (50 mL×3), combined organic layers washed with brine (50 mL), dried over anhydrous sodium sulfate, filtered, concentrated under reduced pressure and purified by silica gel column chromatography (25-35% ethyl acetate/petroleum ether) to give tert-butyl N-[(3R)-1-(5-bromo-2-pyridyl)pyrrolidin-3-yl]carbamate (1.6 g, 81% yield) as a white solid. LCMS (MM-ES+APCI, Pos): m/z 344.0 (M+H).

### Step B. tert-butyl N-[(3R)-1-[5-(4,4,5,5-tetramethyl-1,3,2-dioxaborolan-2-yl)-2-pyridyl]pyrrolidin-3-yl] carbamate

To a 40 mL vial equipped with a magnetic stir bar was added tert-butyl N-[(3R)-1-(5-bromo-2-pyridyl) pyrrolidin-3-yl] carbamate (1.0 g, 2.9 mmol) and 4,4,5,5-tetramethyl-2-(4,4,5,5-tetramethyl-1,3,2-dioxaborolan-2-yl)-1,3,2-dioxaborolane (1.1 g, 4.3 mmol) followed by the addition of dioxane (15 mL). KOAc (0.57 g, 5.8 mmol) and Pd(dppf)Cl₂ (0.22 g, 0.29 mmol) were added into the mixture and stirred at 90° C. under atmosphere of nitrogen for 1 h. The suspension was filtered, and the filter cake was washed with ethyl acetate (30 mL). The crude product was purified by silica gel column chromatography (20-25% ethyl acetate/petroleum ether) to give tert-butyl N-[(3R)-1-[5-(4,4,5,5-tetramethyl-1,3,2-dioxaborolan-2-yl)-2-pyridyl] pyrrolidin-3-yl] carbamate (0.70 g, 56% yield) as a brown oil. LCMS (MM-ES+APCI, Pos): m/z 308.4 (M+H).

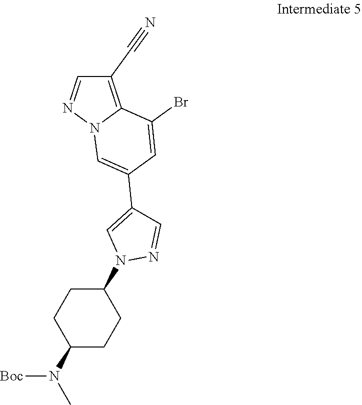 [SMILES: CN(C(=O)OC(C)(C)C)[C@H]1CC[C@@H](n2cc(-c3cc(Br)c4c(C#N)cnn4c3)cn2)CC1]

### tert-butyl N-[4-[4-(4-bromo-3-cyano-pyrazolo[1,5-a]pyridin-6-yl)pyrazol-1-yl]cyclohexyl]-N-methyl-carbamate

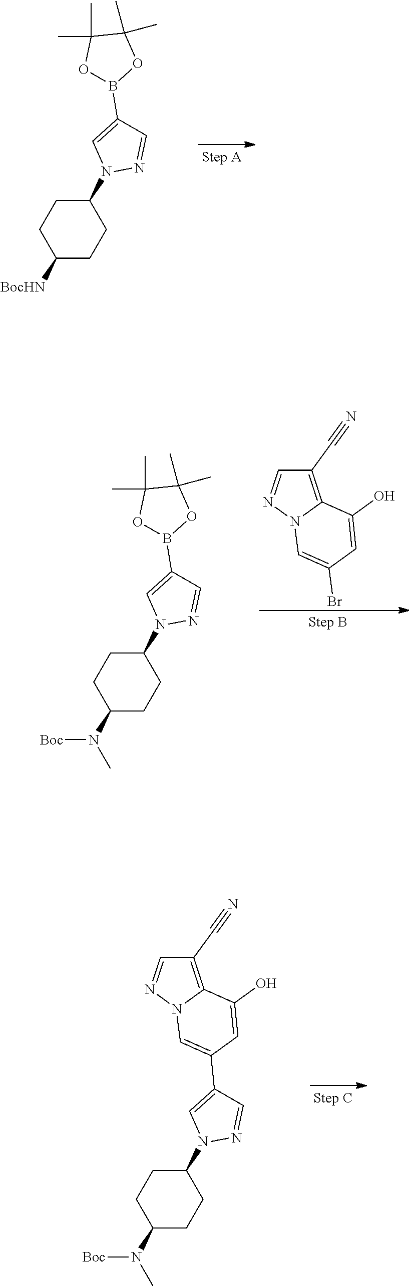 [SMILES: CC(C)(C)OC(=O)N[C@H]1CC[C@@H](n2cc(B3OC(C)(C)C(C)(C)O3)cn2)CC1 ; CN(C(=O)OC(C)(C)C)[C@H]1CC[C@@H](n2cc(B3OC(C)(C)C(C)(C)O3)cn2)CC1 ; N#Cc1cnn2cc(Br)cc(O)c12 ; CN(C(=O)OC(C)(C)C)[C@H]1CC[C@@H](n2cc(-c3cc(O)c4c(C#N)cnn4c3)cn2)CC1]

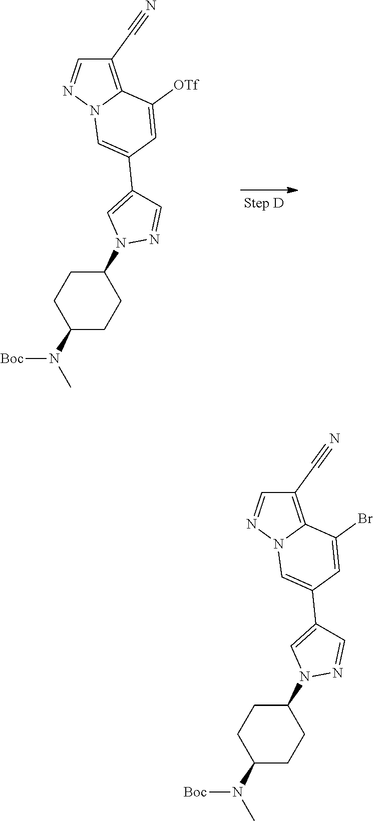 [SMILES: CN(C(=O)OC(C)(C)C)[C@H]1CC[C@@H](n2cc(-c3cc(OS(=O)(=O)C(F)(F)F)c4c(C#N)cnn4c3)cn2)CC1 ; CN(C(=O)OC(C)(C)C)[C@H]1CC[C@@H](n2cc(-c3cc(Br)c4c(C#N)cnn4c3)cn2)CC1]

### Step A. tert-butyl N-methyl-N-[4-[4-(4,4,5,5-tetramethyl-1,3,2-dioxaborolan-2-yl)pyrazol-1-yl]cyclohexyl]carbamate

To a 100 mL round-bottom flask equipped with a magnetic stir bar was added tert-butyl N-[4-[4-(4,4,5,5-tetramethyl-1,3,2-dioxaborolan-2-yl)pyrazol-1-yl]cyclohexyl] carbamate (1.0 g, 2.4 mmol) followed by the addition of THF (12 mL). The flask was cooled to 0° C., evacuated and backfilled with nitrogen three times. NaH (60%, 0.19 g, 4.8 mmol) was added into the mixture in portions at 0° C., followed by addition of CH₃I (0.29 mL, 4.8 mmol) in THF (2 mL) at 0° C. and the reaction stirred at 25° C. for 12 h. The mixture was quenched with saturated aqueous ammonium chloride (10 mL), transferred to a separatory funnel and the aqueous layer extracted with ethyl acetate (10 mL×3). The combined organic layers were washed with brine (10 mL), dried over anhydrous sodium sulfate, filtered, concentrated under reduced pressure and purified by silica gel column chromatography (0-20% ethyl acetate/petroleum ether) to give the tert-butyl N-methyl-N-[4-[4-(4,4,5,5-tetramethyl-1,3,2-dioxaborolan-2-yl)pyrazol-1-yl]cyclohexyl]carbamate (0.45 g, 37% yield) as a white solid. LCMS (MM-ES+APCI, Pos): m/z 406.1 (M+H).

### Step B. tert-butyl N-[4-[4-(3-cyano-4-hydroxy-pyrazolo[1,5-a]pyridin-6-yl)pyrazol-1-yl]cyclohexyl]-N-methyl-carbamate

Made according to Intermediate 2, Step A, substituting tert-butyl N-methyl-N-[4-[4-(4,4,5,5-tetramethyl-1,3,2-dioxaborolan-2-yl)pyrazol-1-yl]cyclohexyl]carbamate for tert-butyl (3S)-3-[4-(4,4,5,5-tetramethyl-1,3,2-dioxaborolan-2-yl)pyrazol-1-yl]piperidine-1-carboxylate to give tert-butyl N-[4-[4-(3-cyano-4-hydroxy-pyrazolo[1,5-a]pyridin-6-yl)pyrazol-1-yl]cyclohexyl]-N-methyl-carbamate (0.30 g, 82% yield) as a yellow oil.

### Step C. [6-[1-[4-[tert-butoxycarbonyl(methyl) amino]cyclohexyl] pyrazol-4-yl]-3-cyano-pyrazolo[1,5-a]pyridin-4-yl] trifluoromethanesulfonate

Made according to Intermediate 2, Step B, substituting tert-butyl N-[4-[4-(3-cyano-4-hydroxy-pyrazolo[1,5-a]pyridin-6-yl)pyrazol-1-yl]cyclohexyl]-N-methyl-carbamate for tert-butyl (3S)-3-[4-(3-cyano-4-hydroxy-pyrazolo[1,5-a]pyridin-6-yl)pyrazol-1-yl]piperidine-1-carboxylate to give [6-[1-[4-[tert-butoxycarbonyl(methyl) amino]cyclohexyl] pyrazol-4-yl]-3-cyano-pyrazolo[1,5-a]pyridin-4-yl] trifluoromethanesulfonate (0.35 g, 84% yield) as a yellow oil. LCMS (MM-ES+APCI, Pos): m/z 569.0 (M+H).

### Step D. tert-butyl N-[4-[4-(4-bromo-3-cyano-pyrazolo[1,5-a]pyridin-6-yl)pyrazol-1-yl]cyclohexyl]-N-methyl-carbamate

Made according to Intermediate 2, Step C, substituting [6-[1-[4-[tert-butoxycarbonyl(methyl)amino]cyclohexyl]pyrazol-4-yl]-3-cyano-pyrazolo[1,5-a]pyridin-4-yl] trifluoromethanesulfonate for tert-butyl (3S)-3-[4-[3-cyano-4-(trifluoromethylsulfonyloxy)pyrazolo[1,5-a]pyridin-6-yl]pyrazol-1-yl]piperidine-1-carboxylate to give tert-butyl N-[4-[4-(4-bromo-3-cyano-pyrazolo[1,5-a]pyridin-6-yl)pyrazol-1-yl]cyclohexyl]-N-methyl-carbamate (0.13 g, 41% yield) as a white solid. LCMS (MM-ES+APCI, Pos): m/z 499.2 (M+H).

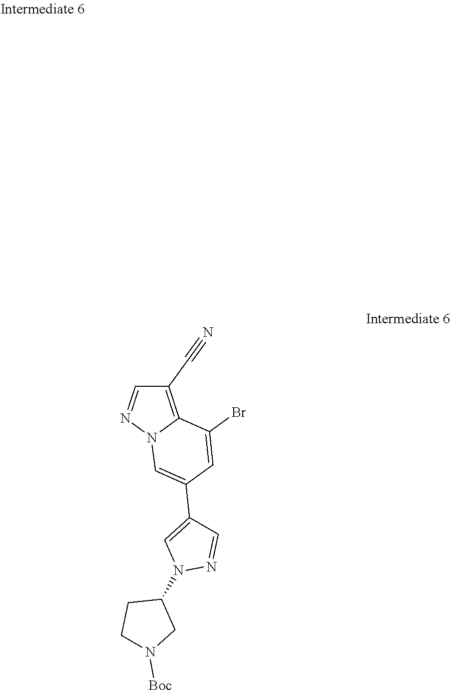 [SMILES: CC(C)(C)OC(=O)N1CC[C@H](n2cc(-c3cc(Br)c4c(C#N)cnn4c3)cn2)C1]

### tert-butyl (S)-3-(4-(4-bromo-3-cyanopyrazolo[1,5-a]pyridin-6-yl)-1H-pyrazol-1-yl)pyrrolidine-1-carboxylate

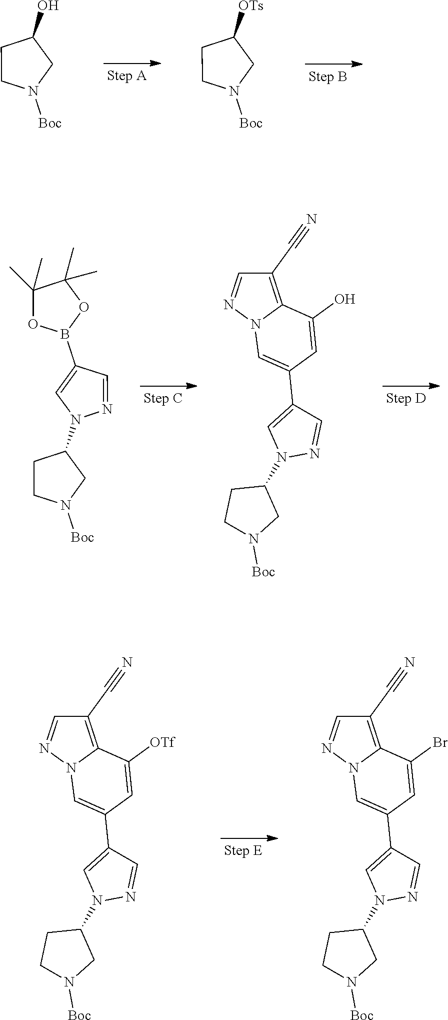 [SMILES: CC(C)(C)OC(=O)N1CC[C@@H](O)C1 ; Cc1ccc(S(=O)(=O)O[C@@H]2CCN(C(=O)OC(C)(C)C)C2)cc1 ; CC(C)(C)OC(=O)N1CC[C@H](n2cc(B3OC(C)(C)C(C)(C)O3)cn2)C1 ; CC(C)(C)OC(=O)N1CC[C@H](n2cc(-c3cc(O)c4c(C#N)cnn4c3)cn2)C1 ; CC(C)(C)OC(=O)N1CC[C@H](n2cc(-c3cc(OS(=O)(=O)C(F)(F)F)c4c(C#N)cnn4c3)cn2)C1 ; CC(C)(C)OC(=O)N1CC[C@H](n2cc(-c3cc(Br)c4c(C#N)cnn4c3)cn2)C1]

### Step A. tert-butyl (R)-3-(tosyloxy)pyrrolidine-1-carboxylate

A solution of tert-butyl (R)-3-hydroxypyrrolidine-1-carboxylate (5.0 g, 27 mmol) and DMAP (0.33 mg, 2.7 mmol) in DCM (67 mL) at 0° ° C. was added triethylamine (7.4 mL, 53 mmol) followed by tosyl chloride (6.1 g, 32 mmol). The reaction was stirred for 1 h at rt. The reaction was diluted with DCM (50 mL) and the organics washed with H₂O (100 mL), brine (100 mL), dried over Na₂SO₄, filtered, concentrated in vacuo and purified by silica gel chromatography (0-50% ethyl acetate/hexanes) to afford tert-butyl (R)-3-(tosyloxy)pyrrolidine-1-carboxylate (4.3 g, 47% yield). LCMS (MM-ES+APCI, Pos): m/z 242.1 (M-Boc+H).

### Step B. tert-butyl (S)-3-(4-(4,4,5,5-tetramethyl-1,3,2-dioxaborolan-2-yl)-1H-pyrazol-1-yl)pyrrolidine-1-carboxylate

To a solution of 4-(4,4,5,5-tetramethyl-1,3,2-dioxaborolan-2-yl)-1H-pyrazole (1.0 g, 5.2 mmol) in DMF (2.6 mL) was added tert-butyl (R)-3-(tosyloxy)pyrrolidine-1-carboxylate (2.6 g, 7.7 mmol) and Cs₂CO₃ (3.4 g, 10 mmol). The solution was stirred at 80° C. for 2 h. The mixture was diluted with EtOAc (100 mL). The organic phase washed with H₂O (100 mL) and the aqueous phase extracted with EtOAc (50 mL). The combined organic phase was washed with brine (3×100 mL), dried with Na₂SO₄, filtered, and concentrated in vacuo to give tert-butyl (S)-3-(4-(4,4,5,5-tetramethyl-1,3,2-dioxaborolan-2-yl)-1H-pyrazol-1-yl)pyrrolidine-1-carboxylate (1.9 g, quant yield), which was used in the next step without further purification. LCMS (MM-ES+APCI, Pos): m/z 364.3 (M+H).

### Step C. tert-butyl (S)-3-(4-(3-cyano-4-hydroxypyrazolo[1,5-a]pyridin-6-yl)-1H-pyrazol-1-yl)pyrrolidine-1-carboxylate

To a solution of tert-butyl (S)-3-(4-(4,4,5,5-tetramethyl-1,3,2-dioxaborolan-2-yl)-1H-pyrazol-1-yl)pyrrolidine-1-carboxylate (1.8 g, 5.0 mmol) in 4:1 dioxane-water (17 mL) was added 6-bromo-4-hydroxypyrazolo[1,5-a]pyridine-3-carbonitrile (0.80 g, 3.4 mmol and K₂CO₃ (0.93 g, 6.7 mmol). The reaction was purged with Argon for 5 minutes. Palladium Tetrakis (0.19 g, 0.17 mmol) was added and the reaction stirred at 80° C. for 7 h. The reaction mixture was diluted with EtOAc (30 mL) and the layers separated. The aqueous phase was extracted with EtOAc (20 mL). The combined organic phase was washed with brine (2×, 50 mL), concentrated in vacuo and purified by silica gel chromatography (0-100% EtOAc/hexanes) to afford tert-butyl (S)-3-(4-(3-cyano-4-hydroxypyrazolo[1,5-a]pyridin-6-yl)-1H-pyrazol-1-yl)pyrrolidine-1-carboxylate (0.39 g, 30% yield). LCMS (MM-ES+APCI, Pos): m/z 339.20 (M-tBu+H).

### Step D. tert-butyl (S)-3-(4-(3-cyano-4-(((trifluoromethyl)sulfonyl)oxy)pyrazolo[1,5-a]pyridin-6-yl)-1H-pyrazol-1-yl)pyrrolidine-1-carboxylate

Made according to Intermediate 2, Step B, substituting tert-butyl (S)-3-(4-(3-cyano-4-hydroxypyrazolo[1,5-a]pyridin-6-yl)-1H-pyrazol-1-yl)pyrrolidine-1-carboxylate for tert-butyl (3S)-3-[4-(3-cyano-4-hydroxy-pyrazolo[1,5-a]pyridin-6-yl)pyrazol-1-yl]piperidine-1-carboxylate to give tert-butyl (S)-3-(4-(3-cyano-4-(((trifluoromethyl)sulfonyl)oxy)pyrazolo[1,5-a]pyridin-6-yl)-1H-pyrazol-1-yl)pyrrolidine-1-carboxylate (67 mg, 95% yield). LCMS (MM-ES+APCI, Pos): m/z 471.1 (M-tBu+H).

### Step E. tert-butyl (S)-3-(4-(4-bromo-3-cyanopyrazolo[1,5-a]pyridin-6-yl)-1H-pyrazol-1-yl)pyrrolidine-1-carboxylate

Made according to Intermediate 2, Step C, substituting tert-butyl (S)-3-(4-(3-cyano-4-(((trifluoromethyl)sulfonyl)oxy)pyrazolo[1,5-a]pyridin-6-yl)-1H-pyrazol-1-yl)pyrrolidine-1-carboxylate for tert-butyl (3S)-3-[4-[3-cyano-4-(trifluoromethylsulfonyloxy)pyrazolo[1,5-a]pyridin-6-yl]pyrazol-1-yl]piperidine-1-carboxylate to give tert-butyl (S)-3-(4-(4-bromo-3-cyanopyrazolo[1,5-a]pyridin-6-yl)-1H-pyrazol-1-yl)pyrrolidine-1-carboxylate (17 mg, 30% yield). LCMS (MM-ES+APCI, Pos): m/z 401.1 (M-tBu+H).

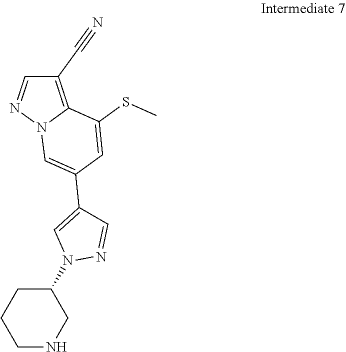 [SMILES: CSc1cc(-c2cnn([C@H]3CCCNC3)c2)cn2ncc(C#N)c12]

### 4-methylsulfanyl-6-[1-[(3S)-3-piperidyl] pyrazol-4-yl] pyrazolo[1,5-a]pyridine-3-carbonitrile

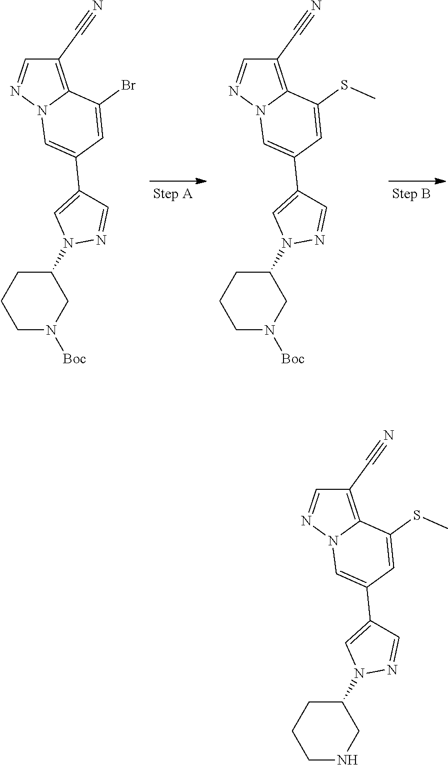 [SMILES: CC(C)(C)OC(=O)N1CCC[C@H](n2cc(-c3cc(Br)c4c(C#N)cnn4c3)cn2)C1 ; CSc1cc(-c2cnn([C@H]3CCCN(C(=O)OC(C)(C)C)C3)c2)cn2ncc(C#N)c12 ; CSc1cc(-c2cnn([C@H]3CCCNC3)c2)cn2ncc(C#N)c12]

### Step A. tert-butyl (3S)-3-[4-(3-cyano-4-methylsulfanyl-pyrazolo[1,5-a]pyridin-6-yl)pyrazol-1-yl]piperidine-1-carboxylate

To a 40 mL vial equipped with a magnetic stir bar was added methylsulfanylsodium (85 mg, 1.2 mmol) followed by the addition of toluene (4 mL). tert-butyl(3S)-3-[4-(4-bromo-3-cyano-pyrazolo[1,5-a]pyridin-6-yl)pyrazol-1-yl]piperidine-1-carboxylate (0.40 g, 0.81 mmol), Pd(OAc)₂ (19 mg, 0.084 mmol), Xantphos (47 mg, 0.081 mmol) and Cs₂CO₃ (0.79 g, 2.4 mmol) were added into the mixture at 25° C. and the reaction stirred at 100° C. under an atmosphere of nitrogen for 16 h. Water (4 mL) was added to the reaction and the mixture was transferred to a separatory funnel. The aqueous layer mixture was extracted with ethyl acetate (5 mL×3). The combined organic layers were washed with brine (10 mL), dried over anhydrous sodium sulfate, filtered, concentrated under reduced and purified by silica gel column chromatography (0-50% EtOAc/petroleum ether) to give tert-butyl (3S)-3-[4-(3-cyano-4-methylsulfanyl-pyrazolo[1,5-a]pyridin-6-yl)pyrazol-1-yl]piperidine-1-carboxylate (0.28 g, 68% yield) as a yellow gum. LCMS (MM-ES+APCI, Pos): m/z 439.1 (M+H).

### Step B. 4-methylsulfanyl-6-[1-[(3S)-3-piperidyl] pyrazol-4-yl] pyrazolo[1,5-a]pyridine-3-carbonitrile

To a 50 mL round-bottom flask equipped with a magnetic stir bar was added tert-butyl (3S)-3-[4-(3-cyano-4-methylsulfanyl-pyrazolo[1,5-a]pyridin-6-yl)pyrazol-1-yl]piperidine-1-carboxylate (60 mg, 0.12 mmol) followed by the addition of DCM (1 mL). HCl/dioxane (4 M, 1 mL) was added to the mixture and the reaction stirred at 25° C. for 1 h. The mixture was concentrated under reduced pressure and purified by preparative HPLC (19%-49% acetonitrile/0.04% aqueous ammonia hydroxide+10 mM NH₄HCO₃). After lyophilization, 4-methylsulfanyl-6-[1-[(3S)-3-piperidyl] pyrazol-4-yl] pyrazolo[1,5-a] pyridine-3-carbonitrile (19.0 mg, 39% yield) was obtained as a white solid. LCMS (MM-ES+APCI, Pos): m/z 339.0 (M+H).

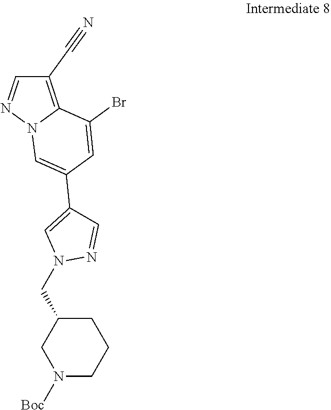 [SMILES: CC(C)(C)OC(=O)N1CCC[C@@H](Cn2cc(-c3cc(Br)c4c(C#N)cnn4c3)cn2)C1]

### tert-butyl (3R)-3-[[4-(4-bromo-3-cyano-pyrazolo[1,5-a]pyridin-6-yl)pyrazol-1-yl]methyl]piperidine-1-carboxylate

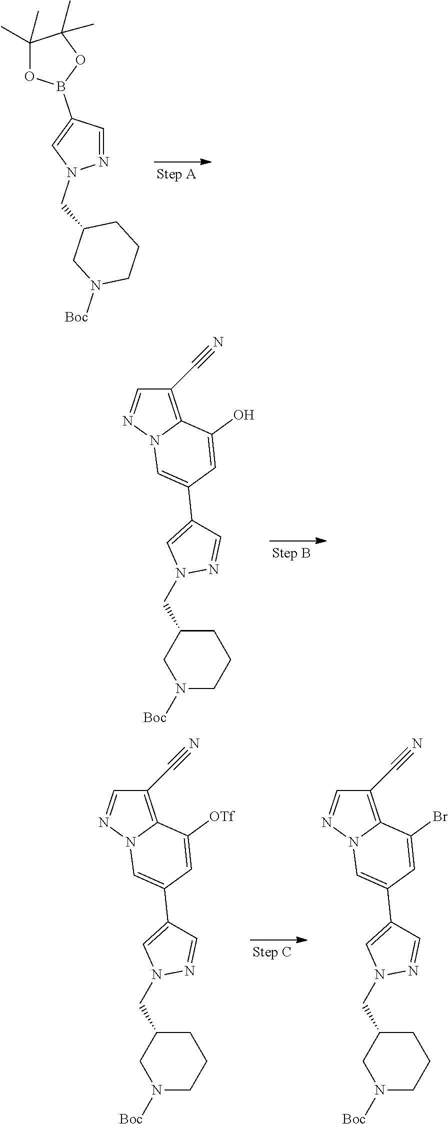

### Step A. tert-butyl (3R)-3-[[4-(3-cyano-4-hydroxy-pyrazolo[1,5-a]pyridin-6-yl)pyrazol-1-yl]methyl]piperidine-1-carboxylate

Made according to Intermediate 2, Step A, substituting tert-butyl (3R)-3-[[4-(4,4,5,5-tetramethyl-1,3,2-dioxaborolan-2-yl)pyrazol-1-yl]methyl]piperidine-1-carboxylate for tert-butyl (3S)-3-[4-(4,4,5,5-tetramethyl-1,3,2-dioxaborolan-2-yl)pyrazol-1-yl]piperidine-1-carboxylate to give tert-butyl (3R)-3-[[4-(3-cyano-4-hydroxy-pyrazolo[1,5-a]pyridin-6-yl)pyrazol-1-yl]methyl]piperidine-1-carboxylate (1.5 g, 76% yield) as a light yellow solid. LCMS (MM-ES+APCI, Pos): m/z 423.2 (M+H).

### Step B. tert-butyl (3R)-3-[[4-[3-cyano-4-(trifluoromethylsulfonyloxy) pyrazolo[1,5-a]pyridin-6-yl]pyrazol-1-yl]methyl]piperidine-1-carboxylate

Made according to Intermediate 2, Step B, substituting tert-butyl (3R)-3-[[4-(3-cyano-4-hydroxy-pyrazolo[1,5-a]pyridin-6-yl)pyrazol-1-yl]methyl]piperidine-1-carboxylate for tert-butyl (3S)-3-[4-(3-cyano-4-hydroxy-pyrazolo[1,5-a]pyridin-6-yl)pyrazol-1-yl]piperidine-1-carboxylate to give tert-butyl (3R)-3-[[4-[3-cyano-4-(trifluoromethylsulfonyloxy) pyrazolo[1,5-a]pyridin-6-yl]pyrazol-1-yl]methyl]piperidine-1-carboxylate (1.7 g, 93% yield) as a yellow solid. LCMS (MM-ES+APCI, Pos): m/z 555.0 (M+H).

### Step C. tert-butyl (3R)-3-[[4-(4-bromo-3-cyano-pyrazolo[1,5-a]pyridin-6-yl)pyrazol-1-yl]methyl]piperidine-1-carboxylate

Made according to Intermediate 2, Step C, substituting tert-butyl (3R)-3-[[4-[3-cyano-4-(trifluoromethylsulfonyloxy)pyrazolo[1,5-a]pyridin-6-yl]pyrazol-1-yl]methyl]piperidine-1-carboxylate for tert-butyl (3S)-3-[4-[3-cyano-4-(trifluoromethylsulfonyloxy)pyrazolo[1,5-a]pyridin-6-yl]pyrazol-1-yl]piperidine-1-carboxylate to give tert-butyl (3R)-3-[[4-(4-bromo-3-cyano-pyrazolo[1,5-a]pyridin-6-yl)pyrazol-1-yl]methyl]piperidine-1-carboxylate (0.76 g, 75% yield) as a gray solid. LCMS (MM-ES+APCI, Pos): m/z 486.9 (M+H).

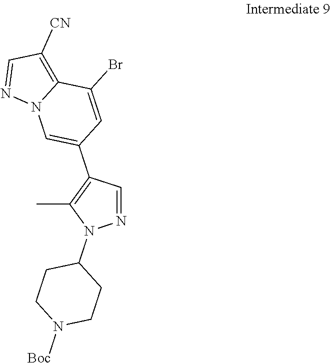 [SMILES: Cc1c(-c2cc(Br)c3c(C#N)cnn3c2)cnn1C1CCN(C(=O)OC(C)(C)C)CC1]

### tert-butyl 4-[4-(4-bromo-3-cyano-pyrazolo[1,5-a]pyridin-6-yl)-5-methyl-pyrazol-1-yl]piperidine-1-carboxylate

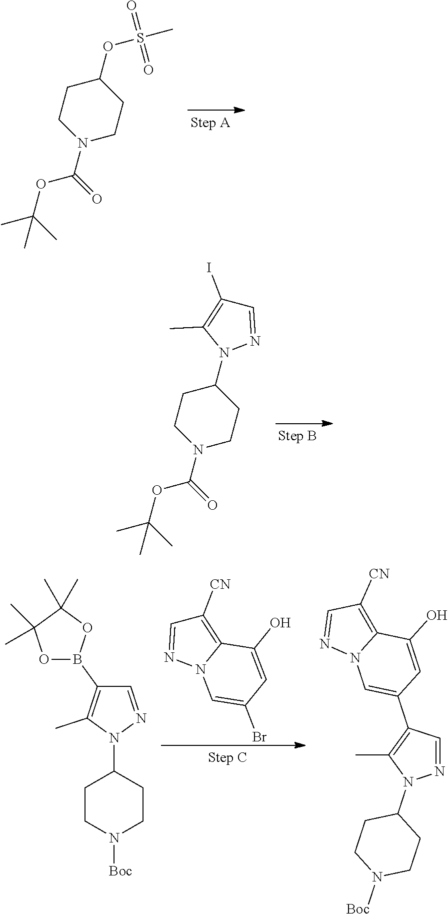 [SMILES: CC(C)(C)OC(=O)N1CCC(OS(C)(=O)=O)CC1 ; Cc1c(I)cnn1C1CCN(C(=O)OC(C)(C)C)CC1 ; Cc1c(B2OC(C)(C)C(C)(C)O2)cnn1C1CCN(C(=O)OC(C)(C)C)CC1 ; N#Cc1cnn2cc(Br)cc(O)c12 ; Cc1c(-c2cc(O)c3c(C#N)cnn3c2)cnn1C1CCN(C(=O)OC(C)(C)C)CC1]

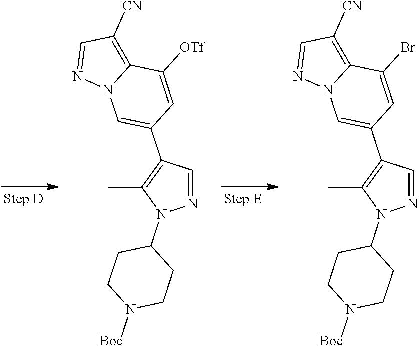 [SMILES: Cc1c(-c2cc(OS(=O)(=O)C(F)(F)F)c3c(C#N)cnn3c2)cnn1C1CCN(C(=O)OC(C)(C)C)CC1 ; Cc1c(-c2cc(Br)c3c(C#N)cnn3c2)cnn1C1CCN(C(=O)OC(C)(C)C)CC1]

### Step A. tert-butyl 4-(4-iodo-5-methyl-pyrazol-1-yl) piperidine-1-carboxylate

To a round-bottom flask equipped with a magnetic stir bar and a reflux condenser was added 4-iodo-5-methyl-1H-pyrazole (10 g, 48 mmol), tert-butyl 4-methylsulfonyloxypiperidine-1-carboxylate (20 g, 72 mmol) and cesium carbonate (31 g, 96 mmol) followed by the addition of N, N-dimethylformamide (300 mL). The mixture was stirred at 90° C. for 4.5 hr. Water (600 mL) was added to the reaction and the mixture was transferred to a separatory funnel. The aqueous layer mixture was extracted with ethyl acetate (200 mL×3). The combined organic layers were washed with brine (800 mL), dried over anhydrous sodium sulfate, filtered, concentrated under reduced pressure and purified by silica gel column chromatography (0-60% EtOAc/petroleum ether) to give tert-butyl 4-(4-iodo-5-methyl-pyrazol-1-yl) piperidine-1-carboxylate (4.4 g, 23% yield) as a white solid.

### Step B. tert-butyl 4-[5-methyl-4-(4, 4, 5, 5-tetramethyl-1, 3, 2-dioxaborolan-2-yl) pyrazol-1-yl]piperidine-1-carboxylate

To a 250 mL round-bottom flask equipped with a magnetic stir bar was added tert-butyl 4-(4-iodo-5-methyl-pyrazol-1-yl) piperidine-1-carboxylate (4.4 g, 11.3 mmol) followed by the addition of THF (120 mL). Isopropylmagnesium chloride (2 M, 23 mL, 45 mmol) was added to the mixture and stirred for 1 h at 25° C. 2-methoxy-4, 4, 5, 5-tetramethyl-1, 3, 2-dioxaborolane (8.9 g, 56 mmol) was added to the mixture and stirred at 25° C. for 3 h. Water (300 mL) was added to the reaction and the mixture was transferred to a separatory funnel. The aqueous mixture was extracted with ethyl acetate (3×100 mL). The combined organic layers were washed with brine (400 mL×3), dried over anhydrous sodium sulfate, filtered, concentrated under reduced pressure and purified by silica gel column chromatography (0-10% EtOAc/petroleum ether) to give tert-butyl 4-[5-methyl-4-(4, 4, 5, 5-tetramethyl-1, 3, 2-dioxaborolan-2-yl) pyrazol-1-yl] piperidine-1-carboxylate (1.9 g, 40% yield) as a white solid. LCMS (MM-ES+APCI, Pos): m/z 392.4 (M+H).

### Step C. tert-butyl 4-[14-(3-cyano-4-hydroxy-pyrazolo[1,5-a]pyridin-6-yl)-5-methyl-pyrazol-1-yl]piperidine-1-carboxylate

Made according to Intermediate 2, Step A, substituting tert-butyl 4-[5-methyl-4-(4, 4, 5, 5-tetramethyl-1, 3, 2-dioxaborolan-2-yl) pyrazol-1-yl] piperidine-1-carboxylate for tert-butyl (3S)-3-[4-(4,4,5,5-tetramethyl-1,3,2-dioxaborolan-2-yl)pyrazol-1-yl]piperidine-1-carboxylate to give tert-butyl 4-[4-(3-cyano-4-hydroxy-pyrazolo[1,5-a]pyridin-6-yl)-5-methyl-pyrazol-1-yl]piperidine-1-carboxylate (10 g, 80% yield) as a white solid. LCMS (MM-ES+APCI, Pos): m/z 367.0 (M-tBu+H).

### Step D. tert-butyl 4-[4-[3-cyano-4-(trifluoromethylsulfonyloxy)pyrazolo[1,5-a]pyridin-6-yl]-5-methyl-pyrazol-1-yl] piperidine-1-carboxylate

Made according to Intermediate 2, Step B, substituting tert-butyl 4-[4-(3-cyano-4-hydroxy-pyrazolo[1,5-a]pyridin-6-yl)-5-methyl-pyrazol-1-yl]piperidine-1-carboxylate for tert-butyl (3S)-3-[4-(3-cyano-4-hydroxy-pyrazolo[1,5-a]pyridin-6-yl)pyrazol-1-yl]piperidine-1-carboxylate to give tert-butyl 4-[4-[3-cyano-4-(trifluoromethylsulfonyloxy)pyrazolo[1,5-a]pyridin-6-yl]-5-methyl-pyrazol-1-yl] piperidine-1-carboxylate (11 g, 85% yield) as a white solid. LCMS (MM-ES+APCI, Pos): m/z 577.1 (M+H).

### Step E. tert-butyl 4-[4-(4-bromo-3-cyano-pyrazolo[1,5-a]pyridin-6-yl)-5-methyl-pyrazol-1-yl]piperidine-1-carboxylate

Made according to Intermediate 2, Step C, substituting tert-butyl 4-[4-[3-cyano-4-(trifluoromethylsulfonyloxy)pyrazolo[1,5-a]pyridin-6-yl]-5-methyl-pyrazol-1-yl]piperidine-1-carboxylate for tert-butyl (3S)-3-[4-[3-cyano-4-(trifluoromethylsulfonyloxy)pyrazolo[1,5-a]pyridin-6-yl]pyrazol-1-yl]piperidine-1-carboxylate to give tert-butyl 4-[4-(4-bromo-3-cyano-pyrazolo[1,5-a]pyridin-6-yl)-5-methyl-pyrazol-1-yl]piperidine-1-carboxylate (11 g, 82% yield) as a white solid. LCMS (MM-ES+APCI, Pos): m/z 430.8 (M+H).

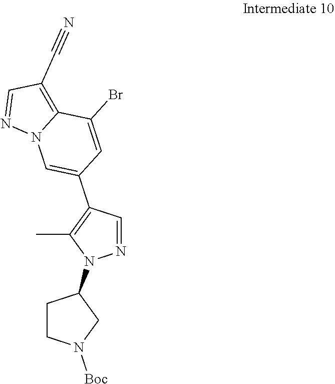 [SMILES: Cc1c(-c2cc(Br)c3c(C#N)cnn3c2)cnn1[C@@H]1CCN(C(=O)OC(C)(C)C)C1]

### tert-butyl (3R)-3-[4-(4-bromo-3-cyano-pyrazolo[1,5-a]pyridin-6-yl)-5-methyl-pyrazol-1-yl]pyrrolidine-1-carboxylate

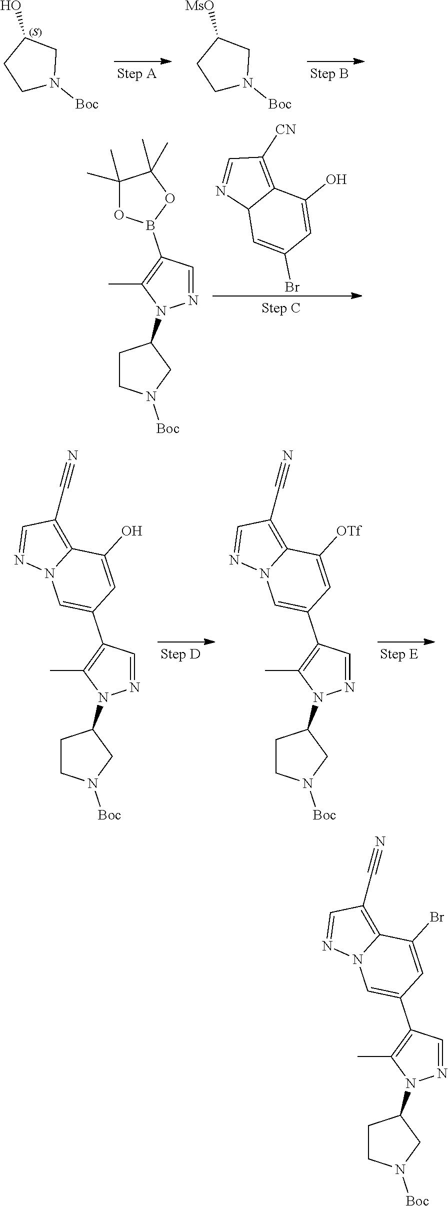

### Step A. tert-butyl (3S)-3-methylsulfonyloxypyrrolidine-1-carboxylate

To a 50 mL round-bottom equipped with a magnetic stir bar were added tert-butyl (3S)-3-hydroxypyrrolidine-1-carboxylate (3.0 g, 16.0 mmol) and triethylamine (4.5 mL, 32 mmol) followed by the addition of DCM (10 mL). The solution was cooled to 0° C. and a solution of methanesulfonic anhydride (3.4 g, 19 mmol) in DCM (10 mL) was added dropwise. Water (20 mL) was added to the reaction and the mixture was transferred to a separatory funnel. The aqueous layer mixture was extracted with DCM (20 mL×5). The combined organic layers were washed with brine (20 mL), dried over anhydrous sodium sulfate, filtered, and concentrated under reduced pressure affording tert-butyl (3S)-3-methylsulfonyloxypyrrolidine-1-carboxylate (3.5 g, 82% yield) as a yellow oil.

### Step B. tert-butyl (3R)-3-[5-methyl-4-(4,4,5,5-tetramethyl-1,3, 2-dioxaborolan-2-yl)pyrazol-1-yl]pyrrolidine-1-carboxylate

To a 100 mL round-bottom flask equipped with a magnetic stir bar and a reflux condenser were added tert-butyl (3S)-3-methylsulfonyloxypyrrolidine-1-carboxylate (2.4 g, 9.1 mmol) and Cs₂CO₃ (6.0 g, 18 mmol) followed by the addition of DMF (30 mL). 5-methyl-4-(4,4,5,5-tetramethyl-1,3,2-dioxaborolan-2-yl)-1H-pyrazole (1.9 g, 9.1 mmol) was added to the mixture at 25° C. The mixture was heated to 90° C. and stirred for 2 h. Saturated ammonium chloride (20 mL) was added to the reaction and the mixture was transferred to a separatory funnel. The aqueous layer mixture was extracted with ethyl acetate (20 mL×3). The combined organic layers were washed with brine (20 mL), dried over anhydrous sodium sulfate, filtered, concentrated under reduced pressure and purified by silica gel column chromatography (28-30% ethyl acetate/petroleum ether) to give tert-butyl (3R)-3-[5-methyl-4-(4,4,5,5-tetramethyl-1,3, 2-dioxaborolan-2-yl)pyrazol-1-yl]pyrrolidine-1-carboxylate (0.48 g, 12% yield) as a white solid. LCMS (MM-ES+APCI, Pos): m/z 378.2 (M+H).

### Step C. tert-butyl (3R)-3-[4-(3-cyano-4-hydroxy-pyrazolo[1,5-a]pyridin-6-yl)-5-methyl-pyrazol-1-yl]pyrrolidine-1-carboxylate

Made according to Intermediate 2, Step A, substituting tert-butyl (3R)-3-[5-methyl-4-(4,4,5,5-tetramethyl-1,3,2-dioxaborolan-2-yl)pyrazol-1-yl]pyrrolidine-1-carboxylate for tert-butyl (3S)-3-[4-(4,4,5,5-tetramethyl-1,3,2-dioxaborolan-2-yl)pyrazol-1-yl]piperidine-1-carboxylate to give tert-butyl (3R)-3-[4-(3-cyano-4-hydroxy-pyrazolo[1,5-a]pyridin-6-yl)-5-methyl-pyrazol-1-yl]pyrrolidine-1-carboxylate (0.42 g, 92% yield) as a yellow solid. LCMS (MM-ES+APCI, Pos): m/z 352.9 (M+H).

### Step D. tert-butyl (3R)-3-[4-[3-cyano-4-(trifluoromethylsulfonyloxy) pyrazolo[1,5-a]pyridin-6-yl]-5-methyl-pyrazol-1-yl]pyrrolidine-1-carboxylate

Made according to Intermediate 2, Step B, substituting tert-butyl (3R)-3-[4-(3-cyano-4-hydroxy-pyrazolo[1,5-a]pyridin-6-yl)-5-methyl-pyrazol-1-yl]pyrrolidine-1-carboxylate for tert-butyl (3S)-3-[4-(3-cyano-4-hydroxy-pyrazolo[1,5-a]pyridin-6-yl)pyrazol-1-yl]piperidine-1-carboxylate to give tert-butyl (3R)-3-[4-[3-cyano-4-(trifluoromethylsulfonyloxy) pyrazolo[1,5-a]pyridin-6-yl]-5-methyl-pyrazol-1-yl]pyrrolidine-1-carboxylate (0.42 g, 74% yield) as a yellow gum. LCMS (MM-ES+APCI, Pos): m/z 484.9 (M+H).

### Step E. tert-butyl (3R)-3-[4-(4-bromo-3-cyano-pyrazolo[1,5-a]pyridin-6-yl)-5-methyl-pyrazol-1-yl]pyrrolidine-1-carboxylate

Made according to Intermediate 2, Step C, substituting tert-butyl (3R)-3-[4-[3-cyano-4-(trifluoromethylsulfonyloxy)pyrazolo[1,5-a]pyridin-6-yl]-5-methyl-pyrazol-1-yl]pyrrolidine-1-carboxylate for tert-butyl (3S)-3-[4-[3-cyano-4-(trifluoromethylsulfonyloxy)pyrazolo[1,5-a]pyridin-6-yl]pyrazol-1-yl]piperidine-1-carboxylate to give tert-butyl (3R)-3-[4-(4-bromo-3-cyano-pyrazolo[1,5-a]pyridin-6-yl)-5-methyl-pyrazol-1-yl]pyrrolidine-1-carboxylate (0.44 g, 95% yield) as a yellow oil. LCMS (MM-ES+APCI, Pos): m/z 416.7 (M+H).

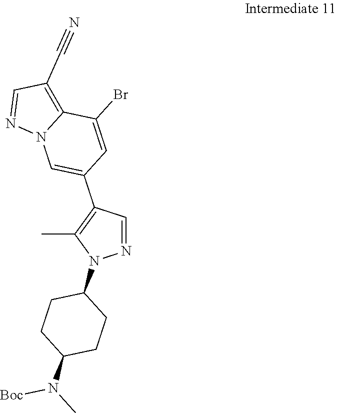 [SMILES: Cc1c(-c2cc(Br)c3c(C#N)cnn3c2)cnn1[C@H]1CC[C@@H](N(C)C(=O)OC(C)(C)C)CC1]

### tert-butyl N-[4-[4-(4-bromo-3-cyano-pyrazolo[1,5-a]pyridin-6-yl)-5-methyl-pyrazol-1-yl]cyclohexyl]-N-methyl-carbamate

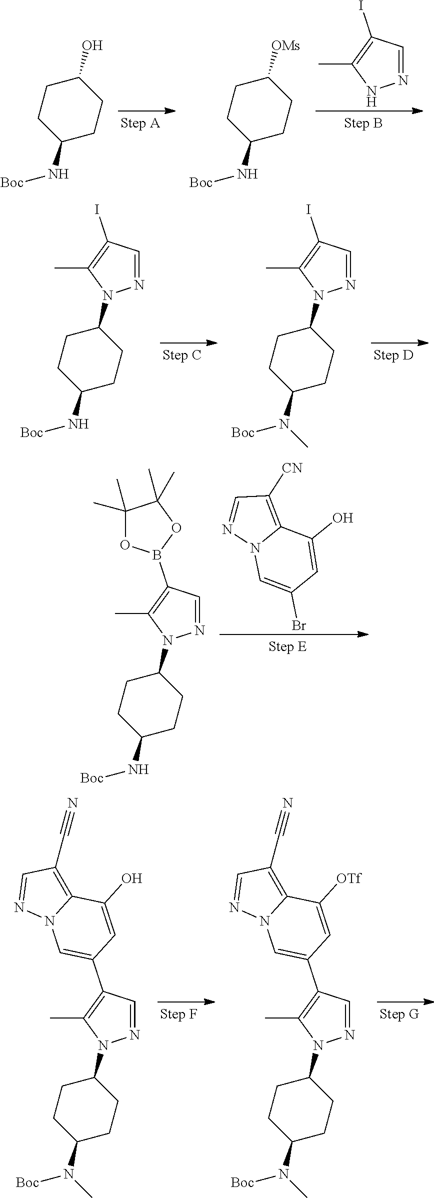

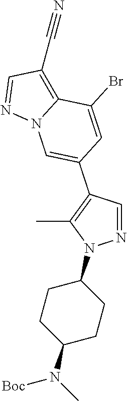 [SMILES: Cc1c(-c2cc(Br)c3c(C#N)cnn3c2)cnn1[C@H]1CC[C@@H](N(C)C(=O)OC(C)(C)C)CC1]

### Step A. 4-(tert-butoxycarbonylamino)cyclohexyl] methanesulfonate

Made according to Intermediate 10, Step A substituting trans tert-butyl N-(4-hydroxycyclohexyl)carbamate for tert-butyl (3S)-3-hydroxypyrrolidine-1-carboxylate to give 4-(tert-butoxycarbonylamino)cyclohexyl] methanesulfonate (13 g, 9% yield) as a yellow solid.

### Step B. tert-butyl N-[4-(4-iodo-5-methyl-pyrazol-1-yl)cyclohexyl]carbamate

Made according to Intermediate 9, Step A substituting trans [4-(t-butoxycarbonylamino)cyclohexyl] methanesulfonate for tert-butyl 4-methylsulfonyloxypiperidine-1-carboxylate to give tert-butyl N-[4-(4-iodo-5-methyl-pyrazol-1-yl)cyclohexyl]carbamate (4.2 g, 31% yield) as a colorless oil.

### Step C. tert-butyl N-[4-(4-iodo-5-methyl-pyrazol-1-yl)cyclohexyl]-N-methyl-carbamate

To a 100 mL round-bottom flask equipped with a magnetic stir bar was added tert-butyl N-[4-(4-iodo-5-methyl-pyrazol-1-yl)cyclohexyl]carbamate (1.2 g, 3.0 mmol) followed by the addition of THF (20 mL). The solution was cooled to 0° C. and treated with NaH (60%, 0.24 g, 5.9 mmol). After stirring at 0° C. for 1 h, CH₃I (0.55 mL, 8.9 mmol) was added, the reaction warmed to 25° C. and stirred for 12 h. Water (30 mL) was added to the reaction and the mixture was transferred to a separatory funnel. The mixture was extracted with ethyl acetate (30 mL×3). The combined organic layer was washed with brine (30 mL), dried over anhydrous sodium sulfate, filtered, concentrated under reduced pressure and purified by silica gel column chromatography (0-70% EtOAc/petroleum ether) to give tert-butyl N-[4-(4-iodo-5-methyl-pyrazol-1-yl)cyclohexyl]-N-methyl-carbamate (1.2 g, 97% yield) as a yellow solid.

### Step D. tert-butyl N-methyl-N-[4-[5-methyl-4-(4,4,5,5-tetramethyl-1,3,2-dioxaborolan-2-yl)pyrazol-1-yl]cyclohexyl]carbamate

Made according to Intermediate 9, Step A substituting cis tert-butyl N-[4-(4-iodo-5-methyl-pyrazol-1-yl)cyclohexyl]-N-methyl-carbamate for tert-butyl 4-(4-iodo-5-methyl-pyrazol-1-yl) piperidine-1-carboxylate to give tert-butyl N-methyl-N-[4-[5-methyl-4-(4,4,5,5-tetramethyl-1,3,2-dioxaborolan-2-yl)pyrazol-1-yl]cyclohexyl]carbamate (1.1 g, 85% yield) as a white solid.

### Step E. tert-butyl N-[4-[4-(3-cyano-4-hydroxy-pyrazolo[1,5-a]pyridin-6-yl)-5-methyl-pyrazol-1-yl]cyclohexyl]-N-methyl-carbamate

Made according to Intermediate 2, Step A, substituting cis tert-butyl N-methyl-N-[4-[5-methyl-4-(4,4,5,5-tetramethyl-1,3,2-dioxaborolan-2-yl)pyrazol-1-yl]cyclohexyl]carbamate for tert-butyl (3S)-3-[4-(4,4,5,5-tetramethyl-1,3,2-dioxaborolan-2-yl)pyrazol-1-yl]piperidine-1-carboxylate to give tert-butyl N-[4-[4-(3-cyano-4-hydroxy-pyrazolo[1,5-a]pyridin-6-yl)-5-methyl-pyrazol-1-yl]cyclohexyl]-N-methyl-carbamate (0.60 g, 53% yield) as a brown solid.

### Step F. [6-[1-[4-[tert-butoxycarbonyl(methyl)amino]cyclohexyl]-5-methyl-pyrazol-4-yl]-3-cyano-pyrazolo[1,5-a]pyridin-4-yl] trifluoromethanesulfonate

Made according to Intermediate 2, Step B, substituting cis tert-butyl N-[4-[4-(3-cyano-4-hydroxypyrazolo[1,5-a]pyridin-6-yl)-5-methyl-pyrazol-1-yl]cyclohexyl]-N-methyl-carbamate for tert-butyl (3S)-3-[4-(3-cyano-4-hydroxy-pyrazolo[1,5-a]pyridin-6-yl)pyrazol-1-yl]piperidine-1-carboxylate to give [6-[1-[4-[tert-butoxycarbonyl(methyl)amino]cyclohexyl]-5-methyl-pyrazol-4-yl]-3-cyano-pyrazolo[1,5-a]pyridin-4-yl] trifluoromethanesulfonate (0.60 g, 81% yield) as a yellow oil. LCMS (MM-ES+APCI, Pos): m/z 583.4 (M+H).

### Step G. tert-butyl N-[4-[4-(4-bromo-3-cyano-pyrazolo[1,5-a]pyridin-6-yl)-5-methyl-pyrazol-1-yl]cyclohexyl]-N-methyl-carbamate

Made according to Intermediate 2, Step C, substituting cis [6-[1-[4-[tert-butoxycarbonyl(methyl)amino]cyclohexyl]-5-methyl-pyrazol-4-yl]-3-cyano-pyrazolo[1,5-a]pyridin-4-yl] trifluoromethanesulfonate for tert-butyl (3S)-3-[4-[3-cyano-4-(trifluoromethylsulfonyloxy)pyrazolo[1,5-a]pyridin-6-yl]pyrazol-1-yl]piperidine-1-carboxylate to give tert-butyl N-[4-[4-(4-bromo-3-cyano-pyrazolo[1,5-a]pyridin-6-yl)-5-methyl-pyrazol-1-yl]cyclohexyl]-N-methyl-carbamate (0.34 g, 68% yield) was obtained as a white solid. LCMS (MM-ES+APCI, Pos): m/z 515.0 (M+H).

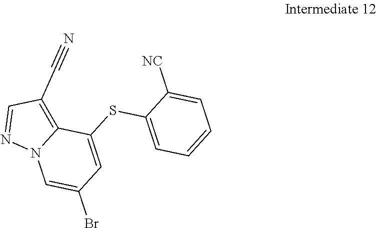 [SMILES: N#Cc1ccccc1Sc1cc(Br)cn2ncc(C#N)c12]

### 6-bromo-4-(2-cyanophenyl)sulfanyl-pyrazolo[1,5-a]pyridine-3-carbonitrile

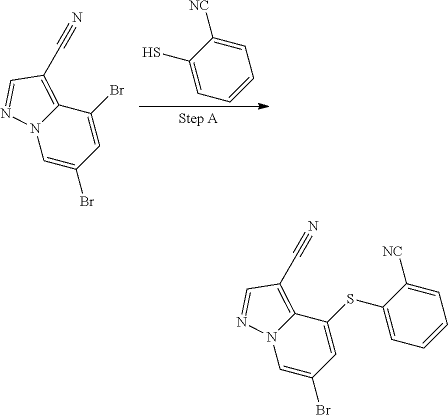 [SMILES: N#Cc1cnn2cc(Br)cc(Br)c12 ; N#Cc1ccccc1S ; N#Cc1ccccc1Sc1cc(Br)cn2ncc(C#N)c12]

### Step A. 6-bromo-4-(2-cyanophenyl)sulfanyl-pyrazolo[1,5-a]pyridine-3-carbonitrile

To a 8 mL vial equipped with a magnetic stir bar was added 4,6-dibromopyrazolo[1,5-a]pyridine-3-carbonitrile (0.50 g, 1.6 mmol) followed by the addition of DMF (4 mL). Methanesulfonato{[4-(N,N-dimethylamino)phenyl]di-t-butylphosphino} (2′-amino-1,1′-biphenyl-2-yl)palladium(II) (10 mg, 0.016 mmol), K₂CO₃ (0.44 g, 3.2 mmol) and 2-sulfanylbenzonitrile (0.17 g, 1.3 mmol) were added into the mixture at 25° C. The flask was purged with nitrogen and stirred at 100° C. for 2 h. Water (10 mL) was added to the reaction and the mixture was transferred to a separatory funnel and the aqueous layer mixture was extracted with ethyl acetate (15 mL×3). The combined organic layers were washed with brine (10 mL), dried over anhydrous sodium sulfate, filtered, concentrated under reduced pressure and purified by silica gel column chromatography (20-40% ethyl acetate/petroleum ether) to give 6-bromo-4-(2-cyanophenyl)sulfanyl-pyrazolo[1,5-a]pyridine-3-carbonitrile (0.85 g, 72% yield) as a light yellow solid. LCMS (MM-ES+APCI, Pos): m/z 355.0 (M+H).

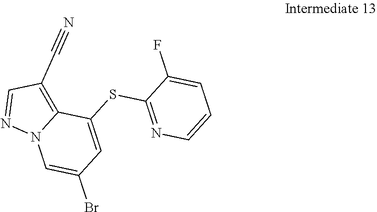 [SMILES: N#Cc1cnn2cc(Br)cc(Sc3ncccc3F)c12]

### 6-bromo-4-[(3-fluoro-2-pyridyl)sulfanyl]pyrazolo[1,5-a]pyridine-3-carbonitrile

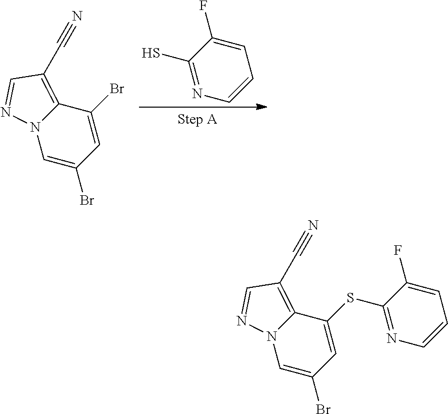 [SMILES: N#Cc1cnn2cc(Br)cc(Br)c12 ; Fc1cccnc1S ; N#Cc1cnn2cc(Br)cc(Sc3ncccc3F)c12]

### Step A. 6-bromo-4-[(3-fluoro-2-pyridyl)sulfanyl]pyrazolo[1,5-a]pyridine-3-carbonitrile

To a 40 mL vial equipped with a magnetic stir bar was added 4,6-dibromopyrazolo[1,5-a]pyridine-3-carbonitrile (2.5 g, 8.3 mmol), Xantphos-Pd-G₃ (0.47 g, 0.49 mmol) and K₂CO₃ (2.5 g, 18 mmol) followed by the addition of DMF (20 mL). 3-fluoropyridine-2-thiol (1.2 g, 9.3 mmol) was added to the mixture at 25° C. The vial was purged with nitrogen for 2 mins. The mixture was heated to 90° C. and stirred for 1 h. Water (50 mL) was added to the reaction and the mixture was transferred to a separatory funnel. The aqueous layer was extracted with ethyl acetate (50 mL×3). The combined organic layers were washed with brine (50 mL), dried over anhydrous sodium sulfate, filtered, and purified by slurry in MeOH (20 mL) at 25° C. for 0.5 h. After filtration and drying under vacuum, 6-bromo-4-[(3-fluoro-2-pyridyl)sulfanyl]pyrazolo[1,5-a]pyridine-3-carbonitrile (2.2 g, 71% yield) was obtained as a yellow solid. LCMS (MM-ES+APCI, Pos): m/z 349.3 (M+H).

### V Methods OF Use

### FGFR Wild Type and Mutant Enzyme Assays

Activity against FGFR wild type and mutant enzymes were determined in biochemical assays using purified intracellular domains of the respective isoforms using HTRF KinEASE-TK assay technology from CisBio, now PerkinElmer. (Catalogue number 62TK0PEC). Briefly test compounds were tested in 11 point 1:3 serial dilutions, typical final concentration ranges from 10,000 nM to 0.17 nM. Test compounds were dissolved in DMSO and added to assay plates using acoustic transfer. Enzyme was pre-incubated with test compound for 30 minutes and the assay was initiated by addition of ATP and TK-peptide substrate. After 60 minutes at room temperature the reaction was quenched with detection reagent and incubated for an additional 60 minutes at room temperature. The plates were read using a multimode reader and the emission ratio of 665 nm to 615 nm was determined. This ratio was converted to percent of control (POC). [1-(high control-sample signal)/(high control-low control)]*100. Wells without compound were used to determine high control values and wells without enzyme were used to determine low control values. The POC values were fit using a four-parameter logistic model and the value where the fit curve was equal to 50 POC was reported as the IC50 using the Signals Vitro Vivo software package (PerkinElmer). Final assay conditions were: 50 mM HEPES pH7.5, 10 mM MgCl2, 1 mM EDTA, 0.01% Brij-35, 2 mM MnCl2, 1 mM DTT, and 0.5 uM TK-peptide. The enzyme source, catalogue number, final enzyme concentration, and final ATP concentration can be found in the tables below.

TABLE 1
A summary of FGFT wild type enzyme assay

| Enzyme | FGFR1 | FGFR2 | FGFR3 | FGFR4 |
|---|---|---|---|---|
| Enzyme | Invitrogen | Carna | Carna | Carna |
| source | | Bioscience | Bioscience | Bioscience |
| Catalog | PV4105 | 08-134 | 08-135 | 08-136 |
| Number | | | | |
| Enzyme conc. | 0.02 nM | 0.03 nM | 0.03 nM | 0.13 nM |
| ATP conc. | 40 uM | 2 uM | 5 uM | 80 uM |

TABLE 2
A summary of FGFT mutant enzyme assay

| | FGFR1 | FGFR2 | FGFR2 | FGFR2 | FGFR3 |
|---|---|---|---|---|---|
| Enzyme | V561M | N549H | V564F | V564I | V555M |
| Enzyme | Carna | Carna | SignalChem | Carna | Carna |
| source | Bioscience | Bioscience | Biotech | Bioscience | Bioscience |
| Catalog | 08-536 | 08-532 | F05-12FG | 08-546 | 08-543 |
| Number | | | | | |
| Enzyme | 0.13 nM | 0.02 nM | 0.02 nM | 0.02 nM | 0.01 nM |
| conc. | | | | | |
| ATP conc. | 1.4 uM | 0.9 uM | 1.5 uM | 0.4 uM | 1.5 uM |

Detection of Phosphorylated FGFR1 (pFGFR1) and FGFR2 (pFGFR2).
Phosphorylated FGFR1 (pFGFR1) Cell Assay:

KG-1 cells were grown in IMDM supplemented with 20% fetal bovine serum. KG-1 cells were plated into a 384-well at 10×10⁵ cells/25 μL/well. Cells were treated with compound using three-fold serial dilutions at final concentrations ranging from 10 μM to 0.5 nM. Compound was incubated on cells for 1 hour at 37° ° C., 5% CO₂.

Following the 1-hour incubation with compound, cell lysates were prepared and phospho FGFR1 was measured using the HTRF Phospho-FGFR1 (TYR653/654) Detection Kits (CisBio, cat #64FGFR1Y6PEG). 8 μL of cell lysis buffer (4×) supplied with 1× Phospho and Total protein blocking reagent (CisBio, cat #64FGFR1Y6PEG, CisBio, cat #64KB1AAC) was then added to the well. Cells were incubated with lysis buffer for 40 minutes at 4° C. under shaking conditions. Plates were centrifuged at 1500 rpm for 3 min then 16 μL of cell lysate was transferred from the cell-culture plate to a small volume detection plate with the added premixed antibody solutions (4 μL) (CisBio, cat #64FGFR1Y6PEG). Plates were centrifuged at 1000 rpm for 1 min then incubated at 25° C. for 120 min, then overnight at 4° ° C. Plates were read on the En Vision multimode plate reader via HTRF dual wavelength detection. One hundred percent of control (POC) was determined using DMSO treated samples, and 0 POC was determined using a control compound. A 4-parameter logistic curve was fit to the POC values as a function of compound concentration and the IC₅₀ value is the point where the curve crosses 50 POC.

Phosphorylated FGFR2-WT (pFGFR2-WT) Cell Assay:

KATOIII cells were grown in the appropriate growth medium, IMDM supplemented with 20% fetal bovine serum. Cells were seeded into a 384-well at 1×10⁵ cells/25 μL/well. Cells were treated with compound using three-fold serial dilutions at final concentrations ranging from 10 μM to 0.5 nM. Compound was incubated on cells for 1 hour at 37° C., 5% CO₂.

Following the 1-hour incubation with compounds, cell lysates were prepared and phospho FGFR2 was measured using the HTRF Phospho-FGFR2 (TYR653/654) Detection Kits (CisBio, cat #64FGFR2Y6PEG). 8 μL of cell lysis buffer (4×) supplied with 1× Phospho and Total protein blocking reagent (CisBio, cat #64FGFR2Y6PEG, CisBio, cat #64KB1AAC) was then added to the wells. Cells were incubated with lysis buffer for 40 minutes at 4° C. under shaking conditions. Plates were centrifuged at 1500 rpm for 3 min and then 16 μL of cell lysate was transferred from the cell-culture plate to a small volume detection plate with the added premixed antibody solutions (4 μL) (CisBio, cat #64FGFR2Y6PEG). Plates were centrifuged at 1000 rpm for 1 min then incubated at 25° ° C. for 120 min, then overnight at 4° C. Plates were read on the EnVision multimode plate reader via HTRF dual wavelength detection. One hundred percent of control (POC) was determined using DMSO treated samples, and 0 POC was determined using the top concentration of a control compound. A 4-parameter logistic curve was fit to the POC values as a function of compound concentration to determine the IC₅₀ value.

Phosphorylated FGFR2-Mutant (pFGFR2-Mutant) Cell Assay:

HEK-293 cells were engineered to express FGFR2-V564F/I/L or FGFR2-Na549K mutations with constructs obtained from GenScript. Cells were grown in appropriate growth medium DMEM supplemented with 10% fetal bovine serum and 150 μg/mL hygromycin. Cells were plated in 96-well poly-D-lysine coated flat bottom plate at 7×10⁵ cells/well and allowed to attached overnight at 37° ° C., 5% CO₂. Cells were treated with compounds using three-fold serial dilutions at final concentrations ranging from 1 μM to 0.05 nM. Compound was incubated on cells for 1 hour at 37° C., 5% CO₂. Cells were stimulated with 100 ng/ml aFGF/bFGF (gibco, cat #13241-013, gibco cat #13256-029) for 10 min at 37° C., 5% CO₂. Medium was removed, and cells were lysed with lysis buffer containing phosphatase and protease inhibitors (Sigma, cat #P8340-5ML, Sigma, cat #P5726-5ML). Phospho FGFR2 was measured by ELISA (R&D Systems, cat #DYC684). Optical density was measured for each well using the BioTek Cytation 5 at wavelength of 450 nm. One hundred percent of control (POC) was determined using DMSO treated samples, and 0 POC was determined using a control compound. A 4-parameter logistic curve was fit to the POC values as a function of compound concentration and the IC₅₀ value is the point where the curve crosses 50 POC.

### VI Examples

The following examples are illustrative in nature and are in no way intended to be limiting.

### Intermediate 1

### Tert-butyl (3S)-3-[4-(4,4,5,5-tetramethyl-1,3,2-dioxaborolan-2-yl)pyrazol-1-yl]piperidine-1-carboxylate

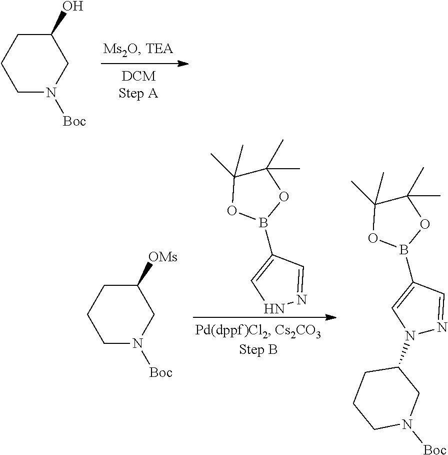 [SMILES: CC(C)(C)OC(=O)N1CCC[C@@H](O)C1 ; CC(C)(C)OC(=O)N1CCC[C@@H](OS(C)(=O)=O)C1 ; CC1(C)OB(c2cn[nH]c2)OC1(C)C ; CC(C)(C)OC(=O)N1CCC[C@H](n2cc(B3OC(C)(C)C(C)(C)O3)cn2)C1]

Step A. Tert-butyl 3-methylsulfonyloxypiperidine-1-carboxylate. To a 40 mL vial flask equipped with a magnetic stir bar was added tert-butyl (3R)-3-hydroxypiperidine-1-carboxylate (0.50 g, 2.5 mmol) followed by the addition of DCM (12 mL). TRIETHYLAMINE (0.69 mL, 5.0 mmol) was added into the mixture. The solution was cooled to 0° C. and methanesulfonic anhydride (0.86 g, 5.0 mmol) was added. The mixture was warmed to 25° C. and stirred for 1 h. Water (10 mL) was added to the reaction and the mixture was transferred to a separatory funnel, the aqueous layer was extracted with ethyl acetate (10 mL×3). The combined organic layers were washed with brine (10 mL), dried over anhydrous sodium sulfate, filtered, and concentrated under reduced pressure affording tert-butyl 3-methylsulfonyloxypiperidine-1-carboxylate (0.90 g, 99% yield) as yellow solid used into the next step without further purification.

Step B. t-Butyl (3S)-3-[4-(4,4,5,5-tetramethyl-1,3,2-dioxaborolan-2-yl)pyrazol-1-yl]piperidine-1-carboxylate. To a 40 mL vial flask equipped with a magnetic stir bar and a reflux condenser was added tert-butyl 3-methylsulfonyloxypiperidine-1-carboxylate (0.65 g, 2.3 mmol) and 4-(4,4,5,5-tetramethyl-1,3,2-dioxaborolan-2-yl)-1H-pyrazole (0.45 g, 2.3 mmol) followed by the addition DMF (12 mL) and Cs₂CO₃ (1.5 g, 4.7 mmol) was added to the mixture. The mixture was heated to 90° C. and stirred for 12 h. Water (10 mL) was added to the reaction and the mixture was transferred to a separatory funnel and the aqueous layer was extracted with ethyl acetate (10 mL×3). The combined organic layer was washed with brine (10 mL), dried over anhydrous sodium sulfate, filtered, concentrated under reduced and purified by silica gel column chromatography (petroleum ether/ethyl acetate from 50/1 to 30/1) to give the desired product tert-butyl (3S)-3-[4-(4,4,5,5-tetramethyl-1,3,2-dioxaborolan-2-yl)pyrazol-1-yl]piperidine-1-carboxylate (0.80 g, 76% yield) as a yellow oil. LCMS (MM-ES+APCI, Pos): m/z 378.1 (M+H).

### Intermediate 2 t-Butyl (3S)-3-[4-(4-bromo-3-cyano-pyrazolo[1,5-a]pyridin-6-yl)pyrazol-1-yl]piperidine-1-carboxylate

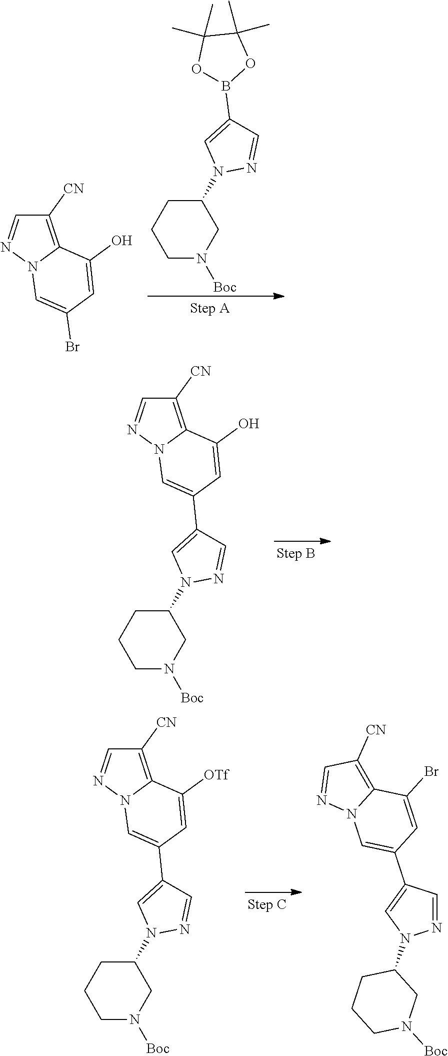

Step A. t-Butyl (3S)-3-[4-(3-cyano-4-hydroxy-pyrazolo[1,5-a]pyridin-6-yl)pyrazol-1-yl]piperidine-1-carboxylate. To a 250 mL round-bottom flask equipped with a magnetic stir bar and a reflux condenser was added 6-bromo-4-hydroxy-pyrazolo[1,5-a]pyridine-3-carbonitrile (6.0 g, 25.2 mmol) and tert-butyl (3S)-3-[4-(4,4,5,5-tetramethyl-1,3,2-dioxaborolan-2-yl)pyrazol-1-yl]piperidine-1-carboxylate (12 g, 30 mmol) followed by the addition of dioxane (60 mL) and H₂O (12 mL). Pd₂(dba)₃ (0.46 mg, 2.5 mmol), XPhos (0.60 g, 1.3 mmol) and K₂CO₃ (7.0 g, 50 mmol) were added. The flask was evacuated and backfilled with nitrogen three times and the reaction was stirred at 90° C. under an atmosphere of nitrogen for 1 h. Water (100 mL) was added to the reaction and the mixture was transferred to a separatory funnel. The aqueous layer was extracted with ethyl acetate (50 mL×3). The combined organic layers were washed with brine (100 mL), dried over anhydrous sodium sulfate, filtered, and concentrated under reduced pressure to a give a brown solid. The solid was purified by silica gel column chromatography (30-100% ethyl acetate/petroleum ether) to give tert-butyl (3S)-3-[4-(3-cyano-4-hydroxy-pyrazolo[1,5-a]pyridin-6-yl)pyrazol-1-yl]piperidine-1-carboxylate (8.5 g, 79% yield) as a brown solid. LCMS (MM-ES+APCI, Pos): m/z 409.1 (M+H).

Step B. t-Butyl (3S)-3-[4-[3-cyano-4-(trifluoromethylsulfonyloxy)pyrazolo[1,5-a]pyridin-6-yl]pyrazol-1-yl]piperidine-1-carboxylate. To a 500 mL round-bottom flask equipped with a magnetic stir bar was added tert-butyl (3S)-3-[4-(3-cyano-4-hydroxy-pyrazolo[1,5-a]pyridin-6-yl)pyrazol-1-yl]piperidine-1-carboxylate (10 g, 24 mmol) and 1,1,1-trifluoro-N-phenyl-N-(trifluoromethylsulfonyl)methanesulfonamide (18 g, 49 mmol) followed by the addition of DMF (150 mL). DIEA (8.5 mL, 49 mmol) was added and the mixture stirred at 25° C. for 2 h. Water (100 mL) was added to the reaction and the mixture was transferred to a separatory funnel, and the aqueous layer was extracted with ethyl acetate (50 mL×3). The combined organic layers were washed with brine (100 mL), dried over anhydrous sodium sulfate, filtered, concentrated under reduced pressure and purified by trituration in ethyl acetate (25 mL) to give tert-butyl (3S)-3-[4-[3-cyano-4-(trifluoromethylsulfonyloxy)pyrazolo[1,5-a]pyridin-6-yl]pyrazol-1-yl]piperidine-1-carboxylate (9.3 g, 87% yield) as a white solid. LCMS (MM-ES+APCI, Pos): m/z 541.3 (M+H).

Step C. t-Butyl (3S)-3-[4-(4-bromo-3-cyano-pyrazolo[1,5-a]pyridin-6-yl)pyrazol-1-yl]piperidine-1-carboxylate. To a 40 mL vial equipped with a magnetic stir bar was added tert-butyl (3S)-3-[4-[3-cyano-4-(trifluoromethylsulfonyloxy)pyrazolo[1,5-a]pyridin-6-yl]pyrazol-1-yl]piperidine-1-carboxylate (1.6 g, 3.0 mmol) and LiBr (0.39 g, 4.4 mmol) followed by the addition of NMP (20 mL). Tris(acetonitrile)pentamethylcyclopentadienyl Ruthenium(II) Trifluoromethanesulfonate (0.10 g, 0.19 mmol) was added, the vial purged with nitrogen and the mixture stirred at 90° C. under an atmosphere of nitrogen for 1 h. The reaction was diluted with water (100 mL) and transferred to a separatory funnel. The aqueous layer was extracted with ethyl acetate (100 mL×3). The combined organic layers were washed with brine (100 mL), dried over anhydrous sodium sulfate, filtered, concentrated under reduced and purified by silica gel column chromatography (30-60% ethyl acetate/petroleum ether) to a brown solid. The solid was purified by trituration with MTBE (30 mL) to give tert-butyl (3S)-3-[4-(4-bromo-3-cyano-pyrazolo[1,5-a]pyridin-6-yl)pyrazol-1-yl]piperidine-1-carboxylate (0.32 g, 63% yield LCMS (MM-ES+APCI, Pos): m/z 473.2 (M+H).

### Intermediate 3 Tert-butyl (3S)-3-[4-(3-cyano-4-sulfanyl-pyrazolo[1,5-a]pyridin-6-yl)pyrazol-1-yl]piperidine-1-carboxylate

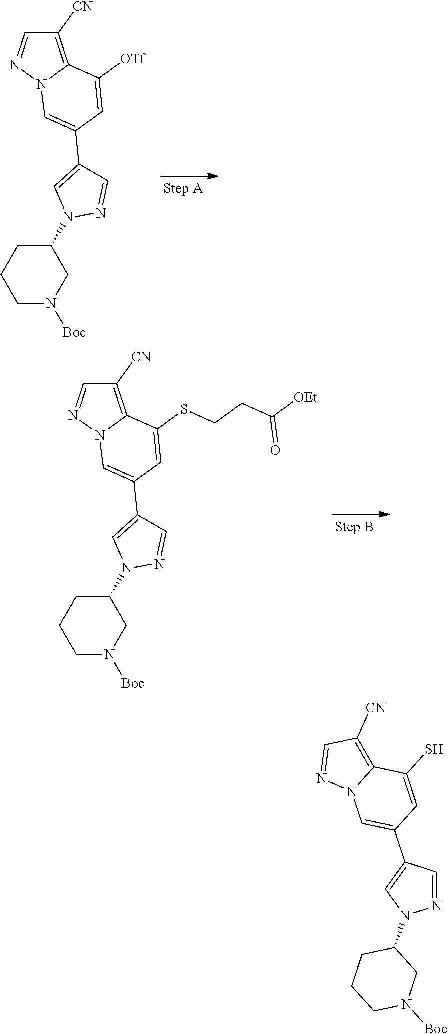 [SMILES: CC(C)(C)OC(=O)N1CCC[C@H](n2cc(-c3cc(OS(=O)(=O)C(F)(F)F)c4c(C#N)cnn4c3)cn2)C1 ; CCOC(=O)CCSc1cc(-c2cnn([C@H]3CCCN(C(=O)OC(C)(C)C)C3)c2)cn2ncc(C#N)c12 ; CC(C)(C)OC(=O)N1CCC[C@H](n2cc(-c3cc(S)c4c(C#N)cnn4c3)cn2)C1]

Step A. t-Butyl (3S)-3-[4-[[3-cyano-4-(3-ethoxy-3-oxo-propyl)sulfanyl-pyrazolo[1,5-a]pyridin-6-yl]pyrazol-1-yl]piperidine-1-carboxylate. To a 40 mL vial equipped with a magnetic stir bar was added tert-butyl (3S)-3-[4-[3-cyano-4-(trifluoromethylsulfonyloxy)pyrazolo[1,5-a]pyridin-6-yl]pyrazol-1-yl]piperidine-1-carboxylate (0.75 g, 1.3 mmol) followed by the addition of toluene (10 mL). Xantphos (74 mg, 0.13 mmol), Pd(OAc)₂ (33 mg, 0.13 mmol), K₃PO₄ (0.54 g, 2.5 mmol) and ethyl 3-sulfanylpropanoate (0.26 g, 1.9 mmol) were added into the mixture at 25° C. The vial was backfilled with nitrogen and stirred at 80° C. under an atmosphere of nitrogen for 16 h. Water (25 mL) was added to the reaction and the mixture was transferred to a separatory funnel. the aqueous layer was extracted with ethyl acetate (25 mL×3). The combined organic layers were washed with brine (80 mL), dried over anhydrous sodium sulfate, filtered, concentrated under reduced pressure and purified by silica gel column chromatography (40-50% ethyl acetate/petroleum ether) to give tert-butyl (3S)-3-[4-[3-cyano-4-(3-ethoxy-3-oxo-propyl)sulfanyl-pyrazolo[1,5-a]pyridin-6-yl]pyrazol-1-yl]piperidine-1-carboxylate (1.3 g, 93% yield) as a yellow gum. LCMS (MM-ES+APCI, Pos): m/z 525.4 (M+H).

Step B. Tert-butyl (3S)-3-[4-(3-cyano-4-sulfany]-pyrazolo[1,5-a]pyridin-6-yl)pyrazol-1-yl]piperidine-1-carboxylate. To a 40 mL vial equipped with a magnetic stir bar was added tert-butyl (3S)-3-[4-[3-cyano-4-(3-ethoxy-3-oxo-propyl)sulfanylpyrazolo[1,5-a]pyridin-6-yl]pyrazol-1-yl]piperidine-1-carboxylate (0.20 g, 0.36 mmol) followed by the addition of DMF (4 mL). The solution was cooled to 0° C. and treated with t-BuOK (1 M, 0.47 mL, 0.47 mmol) dropwise. The mixture was stirred at 0° C. for 1 h. The mixture was concentrated under reduced pressure affording tert-butyl (3S)-3-[4-(3-cyano-4-sulfanyl-pyrazolo[1,5-a]pyridin-6-yl)pyrazol-1-yl]piperidine-1-carboxylate (0.18 g, crude) which was used in the next step without further purification.

### Intermediate 4 Tert-butyl N-[(3R)-1-[5-(4,4,5,5-tetramethyl-1,3,2-dioxaborolan-2-yl)-2-pyridyl] pyrrolidin-3-yl] carbamate

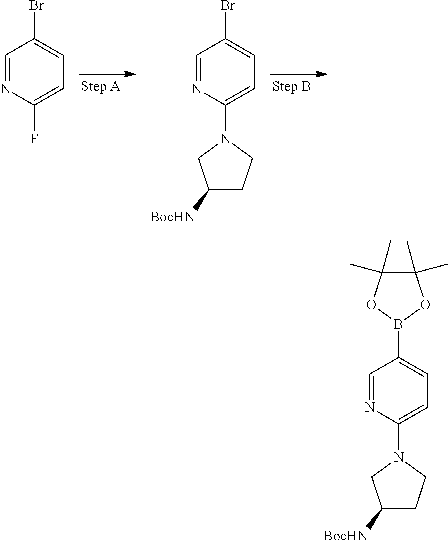

Step A. t-Butyl N-[(3R)-1-(5-bromo-2-pyridyl)pyrrolidin-3-yl]carbamate. To a 40 mL vial equipped with a magnetic stir bar was added tert-butyl N-[(3R)-pyrrolidin-3-yl]carbamate (0.35 g, 1.9 mmol) in DMSO (7 mL). DIEA (1.3 mL, 7.6 mmol) and 5-bromo-2-fluoro-pyridine (0.41 g, 2.3 mmol) were added into the mixture. The vial was purged with nitrogen and heated to 90° C. for 1 h. Water (50 mL) was added to the reaction and the mixture was transferred to a separatory funnel. The aqueous layer was extracted with ethyl acetate (50 mL×3), combined organic layers were washed with brine (50 mL), dried over anhydrous sodium sulfate, filtered, concentrated under reduced pressure and purified by silica gel column chromatography (25-35% ethyl acetate/petroleum ether) to give tert-butyl N-[(3R)-1-(5-bromo-2-pyridyl)pyrrolidin-3-yl]carbamate (1.6 g, 81% yield) as a white solid. LCMS (MM-ES+APCI, Pos): m/z 344.0 (M+H).

Step B. t-Butyl N-[(3R)-1-[5-(4,4,5,5-tetramethyl-1,3,2-dioxaborolan-2-yl)-2-pyridyl]pyrrolidin-3-yl] carbamate. To a 40 mL vial equipped with a magnetic stir bar was added tert-butyl N-[(3R)-1-(5-bromo-2-pyridyl) pyrrolidin-3-yl] carbamate (1.0 g, 2.9 mmol) and 4,4,5,5-tetramethyl-2-(4,4,5,5-tetramethyl-1,3,2-dioxaborolan-2-yl)-1,3,2-dioxaborolane (1.1 g, 4.3 mmol) followed by the addition of dioxane (15 mL). KOAc (0.57 g, 5.8 mmol) and Pd(dppf)Cl₂ (0.22 g, 0.29 mmol) were added into the mixture and stirred at 90° C. under atmosphere of nitrogen for 1 h. The suspension was filtered, and the filter cake was washed with ethyl acetate (30 mL). The crude product was purified by silica gel column chromatography (20-25% ethyl acetate/petroleum ether) to give tert-butyl N-[(3R)-1-[5-(4,4,5,5-tetramethyl-1,3,2-dioxaborolan-2-yl)-2-pyridyl] pyrrolidin-3-yl] carbamate (0.70 g, 56% yield) as a brown oil. LCMS (MM-ES+APCI, Pos): m/z 308.4 (M+H).

### Intermediate 5 t-Butyl N-[4-[4-(4-bromo-3-cyano-pyrazolo[1,5-a]pyridin-6-yl)pyrazol-1-yl]cyclohexyl]-N-methyl-carbamate

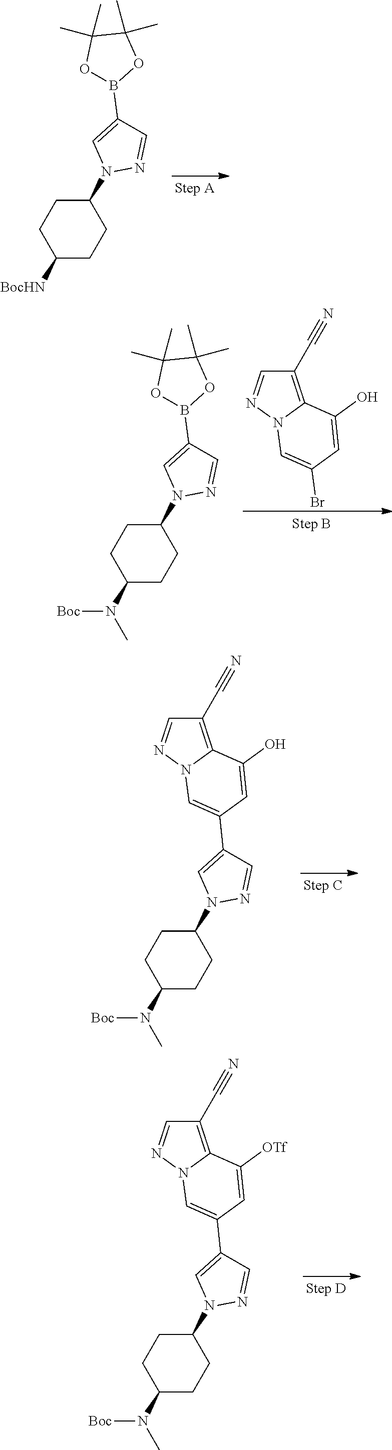

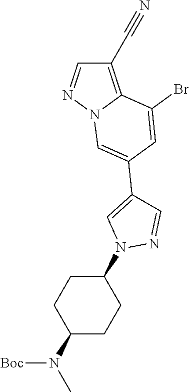 [SMILES: CN(C(=O)OC(C)(C)C)[C@H]1CC[C@@H](n2cc(-c3cc(Br)c4c(C#N)cnn4c3)cn2)CC1]

Step A. t-Butyl N-methyl-N-[4-[4-(4,4,5,5-tetramethyl-1,3,2-dioxaborolan-2-yl)pyrazol-1-yl]cyclohexyl]carbamate. To a 100 mL round-bottom flask equipped with a magnetic stir bar was added tert-butyl N-[4-[4-(4,4,5,5-tetramethyl-1,3,2-dioxaborolan-2-yl)pyrazol-1-yl]cyclohexyl] carbamate (1.0 g, 2.4 mmol) followed by the addition of THF (12 mL). The flask was cooled to 0° C., evacuated and backfilled with nitrogen three times. NaH (60%, 0.19 g, 4.8 mmol) was added into the mixture in portions at 0° C., followed by addition of CH₃I (0.29 mL, 4.8 mmol) in THF (2 mL) at 0° C. and the reaction stirred at 25° C. for 12 h. The mixture was quenched with saturated aqueous ammonium chloride (10 mL), transferred to a separatory funnel and the aqueous layer extracted with ethyl acetate (10 mL×3). The combined organic layers were washed with brine (10 mL), dried over anhydrous sodium sulfate, filtered, concentrated under reduced pressure and purified by silica gel column chromatography (0-20% ethyl acetate/petroleum ether) to give the tert-butyl N-methyl-N-[4-[4-(4,4,5,5-tetramethyl-1,3,2-dioxaborolan-2-yl)pyrazol-1-yl]cyclohexyl]carbamate (0.45 g, 37% yield) as a white solid. LCMS (MM-ES+APCI, Pos): m/z 406.1 (M+H).

Step B. t-Butyl N-[4-[4-(3-cyano-4-hydroxy-pyrazolo[1,5-a]pyridin-6-yl)pyrazol-1-yl]cyclohexyl]-N-methyl-carbamate. Synthesized according to Intermediate 2, Step A, substituting tert-butyl N-methyl-N-[4-[4-(4,4,5,5-tetramethyl-1,3,2-dioxaborolan-2-yl)pyrazol-1-yl]cyclohexyl]carbamate for tert-butyl (3S)-3-[4-(4,4,5,5-tetramethyl-1,3,2-dioxaborolan-2-yl)pyrazol-1-yl]piperidine-1-carboxylate to give tert-butyl N-[4-[4-(3-cyano-4-hydroxy-pyrazolo[1,5-a]pyridin-6-yl)pyrazol-1-yl]cyclohexyl]-N-methyl-carbamate (0.30 g, 82% yield) as a yellow oil.

Step C. [6-[1-[4-[Tert-butoxycarbonyl(methyl) amino]cyclohexyl] pyrazol-4-yl]-3-cyano-pyrazolo[1,5-a]pyridin-4-yl] trifluoromethanesulfonate. Synthesized according to Intermediate 2, Step B, substituting tert-butyl N-[4-[4-(3-cyano-4-hydroxy-pyrazolo[1,5-a]pyridin-6-yl)pyrazol-1-yl]cyclohexyl]-N-methyl-carbamate for tert-butyl (3S)-3-[4-(3-cyano-4-hydroxy-pyrazolo[1,5-a]pyridin-6-yl)pyrazol-1-yl]piperidine-1-carboxylate to give [6-[1-[4-[tert-butoxycarbonyl(methyl) amino]cyclohexyl] pyrazol-4-yl]-3-cyano-pyrazolo[1,5-a]pyridin-4-yl] trifluoromethanesulfonate (0.35 g, 84% yield) as a yellow oil. LCMS (MM-ES+APCI, Pos): m/z 569.0 (M+H).

Step D. t-Butyl N-[4-[4-(4-bromo-3-cyano-pyrazolo[1,5-a]pyridin-6-yl)pyrazol-1-yl]cyclohexyl]-N-methyl-carbamate. Synthesized according to Intermediate 2, Step C, substituting [6-[1-[4-[tert-butoxycarbonyl(methyl)amino]cyclohexyl]pyrazol-4-yl]-3-cyano-pyrazolo[1,5-a]pyridin-4-yl] trifluoromethanesulfonate for tert-butyl (3S)-3-[4-[3-cyano-4-(trifluoromethylsulfonyloxy)pyrazolo[1,5-a]pyridin-6-yl]pyrazol-1-yl]piperidine-1-carboxylate to give tert-butyl N-[4-[4-(4-bromo-3-cyano-pyrazolo[1,5-a]pyridin-6-yl)pyrazol-1-yl]cyclohexyl]-N-methyl-carbamate (0.13 g, 41% yield) as a white solid. LCMS (MM-ES+APCI, Pos): m/z 499.2 (M+H).

### Intermediate 6 t-Butyl (S)-3-(4-(4-bromo-3-cyanopyrazolo[1,5-a]pyridin-6-yl)-1H-pyrazol-1-yl)pyrrolidine-1-carboxylate

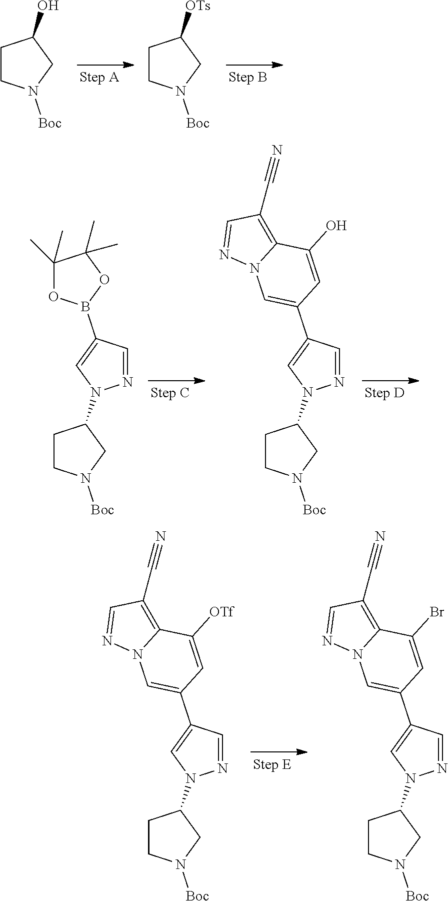 [SMILES: CC(C)(C)OC(=O)N1CC[C@@H](O)C1 ; Cc1ccc(S(=O)(=O)O[C@@H]2CCN(C(=O)OC(C)(C)C)C2)cc1 ; CC(C)(C)OC(=O)N1CC[C@H](n2cc(B3OC(C)(C)C(C)(C)O3)cn2)C1 ; CC(C)(C)OC(=O)N1CC[C@H](n2cc(-c3cc(O)c4c(C#N)cnn4c3)cn2)C1 ; CC(C)(C)OC(=O)N1CC[C@H](n2cc(-c3cc(OS(=O)(=O)C(F)(F)F)c4c(C#N)cnn4c3)cn2)C1 ; CC(C)(C)OC(=O)N1CC[C@H](n2cc(-c3cc(Br)c4c(C#N)cnn4c3)cn2)C1]

Step A. t-Butyl (R)-3-(tosyloxy)pyrrolidine-1-carboxylate. A solution of tert-butyl (R)-3-hydroxypyrrolidine-1-carboxylate (5.0 g, 27 mmol) and DMAP (0.33 mg, 2.7 mmol) in DCM (67 mL) at 0° ° C. was added triethylamine (7.4 mL, 53 mmol) followed by tosyl chloride (6.1 g, 32 mmol). The reaction was stirred for 1 h at rt. The reaction was diluted with DCM (50 mL) and the organics washed with H₂O (100 mL), brine (100 mL), dried over Na₂SO₄, filtered, concentrated in vacuo and purified by silica gel chromatography (0-50% ethyl acetate/hexanes) to afford tert-butyl (R)-3-(tosyloxy)pyrrolidine-1-carboxylate (4.3 g, 47% yield). LCMS (MM-ES+APCI, Pos): m/z 242.1 (M-Boc+H).

Step B. t-Butyl (S)-3-(4-(4,4,5,5-tetramethyl-1,3,2-dioxaborolan-2-yl)-1H-pyrazol-1-yl)pyrrolidine-1-carboxylate. To a solution of 4-(4,4,5,5-tetramethyl-1,3,2-dioxaborolan-2-yl)-1H-pyrazole (1.0 g, 5.2 mmol) in DMF (2.6 mL) was added tert-butyl (R)-3-(tosyloxy)pyrrolidine-1-carboxylate (2.6 g, 7.7 mmol) and Cs₂CO₃ (3.4 g, 10 mmol). The solution was stirred at 80° C. for 2 h. The mixture was diluted with EtOAc (100 mL). The organic phase washed with H₂O (100 mL) and the aqueous phase extracted with EtOAc (50 mL). The combined organic phase was washed with brine (3×100 mL), dried with Na₂SO₄, filtered, and concentrated in vacuo to give tert-butyl (S)-3-(4-(4,4,5,5-tetramethyl-1,3,2-dioxaborolan-2-yl)-1H-pyrazol-1-yl)pyrrolidine-1-carboxylate (1.9 g, quant yield), which was used in the next step without further purification. LCMS (MM-ES+APCI, Pos): m/z 364.3 (M+H).

Step C. t-Butyl (S)-3-(4-(3-cyano-4-hydroxypyrazolo[1,5-a]pyridin-6-yl)-1H-pyrazol-1-yl)pyrrolidine-1-carboxylate. To a solution of tert-butyl (S)-3-(4-(4,4,5,5-tetramethyl-1,3,2-dioxaborolan-2-yl)-1H-pyrazol-1-yl)pyrrolidine-1-carboxylate (1.8 g, 5.0 mmol) in 4:1 dioxane-water (17 mL) was added 6-bromo-4-hydroxypyrazolo[1,5-a]pyridine-3-carbonitrile (0.80 g, 3.4 mmol and K₂CO₃ (0.93 g, 6.7 mmol). The reaction was purged with Argon for 5 minutes. Palladium Tetrakis (0.19 g, 0.17 mmol) was added and the reaction stirred at 80° C. for 7 h. The reaction mixture was diluted with EtOAc (30 mL) and the layers separated. The aqueous phase was extracted with EtOAc (20 mL). The combined organic phase was washed with brine (2×, 50 mL), concentrated in vacuo and purified by silica gel chromatography (0-100% EtOAc/hexanes) to afford tert-butyl (S)-3-(4-(3-cyano-4-hydroxypyrazolo[1,5-a]pyridin-6-yl)-1H-pyrazol-1-yl)pyrrolidine-1-carboxylate (0.39 g, 30% yield). LCMS (MM-ES+APCI, Pos): m/z 339.20 (M-tBu+H).

Step D. t-Butyl (S)-3-(4-(3-cyano-4-(((trifluoromethyl)sulfonyl)oxy)pyrazolo[1,5-a]pyridin-6-yl)-1H-pyrazol-1-yl)pyrrolidine-1-carboxylate. Synthesized according to Intermediate 2, Step B, substituting tert-butyl (S)-3-(4-(3-cyano-4-hydroxypyrazolo[1,5-a]pyridin-6-yl)-1H-pyrazol-1-yl)pyrrolidine-1-carboxylate for tert-butyl (3S)-3-[4-(3-cyano-4-hydroxy-pyrazolo[1,5-a]pyridin-6-yl)pyrazol-1-yl]piperidine-1-carboxylate to give tert-butyl (S)-3-(4-(3-cyano-4-(((trifluoromethyl)sulfonyl)oxy)pyrazolo[1,5-a]pyridin-6-yl)-1H-pyrazol-1-yl)pyrrolidine-1-carboxylate (67 mg, 95% yield). LCMS (MM-ES+APCI, Pos): m/z 471.1 (M-tBu+H).

Step E. t-Butyl (S)-3-(4-(4-bromo-3-cyanopyrazolo[1,5-a]pyridin-6-yl)-1H-pyrazol-1-yl)pyrrolidine-1-carboxylate. Synthesized according to Intermediate 2, Step C, substituting tert-butyl (S)-3-(4-(3-cyano-4-(((trifluoromethyl)sulfonyl)oxy)pyrazolo[1,5-a]pyridin-6-yl)-1H-pyrazol-1-yl)pyrrolidine-1-carboxylate for tert-butyl (3S)-3-[4-[3-cyano-4-(trifluoromethylsulfonyloxy)pyrazolo[1,5-a]pyridin-6-yl]pyrazol-1-yl]piperidine-1-carboxylate to give tert-butyl (S)-3-(4-(4-bromo-3-cyanopyrazolo[1,5-a]pyridin-6-yl)-1H-pyrazol-1-yl)pyrrolidine-1-carboxylate (17 mg, 30% yield). LCMS (MM-ES+APCI, Pos): m/z 401.1 (M-tBu+H).

### Intermediate 7

### 4-Methylsulfanyl-6-[1-[(3S)-3-piperidyl] pyrazol-4-yl] pyrazolo[1,5-a]pyridine-3-carbonitrile

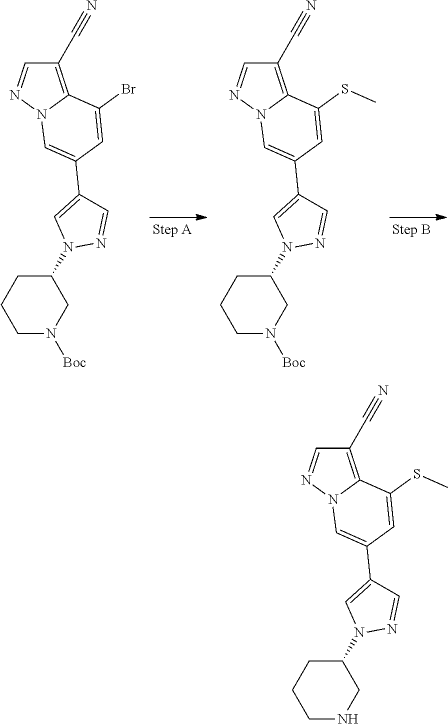 [SMILES: CC(C)(C)OC(=O)N1CCC[C@H](n2cc(-c3cc(Br)c4c(C#N)cnn4c3)cn2)C1 ; CSc1cc(-c2cnn([C@H]3CCCN(C(=O)OC(C)(C)C)C3)c2)cn2ncc(C#N)c12 ; CSc1cc(-c2cnn([C@H]3CCCNC3)c2)cn2ncc(C#N)c12]

Step A. t-Butyl (3S)-3-[4-(3-cyano-4-methylsulfanyl-pyrazolo[1,5-a]pyridin-6-yl)pyrazol-1-yl]piperidine-1-carboxylate. To a 40 mL vial equipped with a magnetic stir bar was added methylsulfanylsodium (85 mg, 1.2 mmol) followed by the addition of toluene (4 mL). t-Butyl(3S)-3-[4-(4-bromo-3-cyano-pyrazolo[1,5-a]pyridin-6-yl)pyrazol-1-yl]piperidine-1-carboxylate (0.40 g, 0.81 mmol), Pd(OAc)₂ (19 mg, 0.084 mmol), xantphos (47 mg, 0.081 mmol) and Cs₂CO₃ (0.79 g, 2.4 mmol) were added into the mixture at 25° C. and the reaction stirred at 100° ° C. under an atmosphere of nitrogen for 16 h. Water (4 mL) was added to the reaction and the mixture was transferred to a separatory funnel. The aqueous layer mixture was extracted with ethyl acetate (5 mL×3). The combined organic layers were washed with brine (10 mL), dried over anhydrous sodium sulfate, filtered, concentrated under reduced and purified by silica gel column chromatography (0-50% EtOAc/petroleum ether) to give tert-butyl (3S)-3-[4-(3-cyano-4-methylsulfanyl-pyrazolo[1,5-a]pyridin-6-yl)pyrazol-1-yl]piperidine-1-carboxylate (0.28 g, 68% yield) as a yellow gum. LCMS (MM-ES+APCI, Pos): m/z 439.1 (M+H).

Step B. 4-Methylsulfanyl-6-[1-[(3S)-3-piperidyl] pyrazol-4-yl] pyrazolo[1,5-a]pyridine-3-carbonitrile. To a 50 mL round-bottom flask equipped with a magnetic stir bar was added tert-butyl (3S)-3-[4-(3-cyano-4-methylsulfanyl-pyrazolo[1,5-a]pyridin-6-yl)pyrazol-1-yl]piperidine-1-carboxylate (60 mg, 0.12 mmol) followed by the addition of DCM (1 mL). HCl/dioxane (4 M, 1 mL) was added to the mixture and the reaction stirred at 25° C. for 1 h. The mixture was concentrated under reduced pressure and purified by preparative HPLC (19%-49% acetonitrile/0.04% aqueous ammonia hydroxide+10 mM NH₄HCO₃). After lyophilization, 4-methylsulfanyl-6-[1-[(3S)-3-piperidyl] pyrazol-4-yl] pyrazolo[1,5-a] pyridine-3-carbonitrile (19.0 mg, 39% yield) was obtained as a white solid. LCMS (MM-ES+APCI, Pos): m/z 339.0 (M+H).

### Intermediate 8

### t-Butyl (3R)-3-[[4-(4-bromo-3-cyano-pyrazolo[1,5-a]pyridin-6-yl)pyrazol-1-yl]methyl]piperidine-1-carboxylate

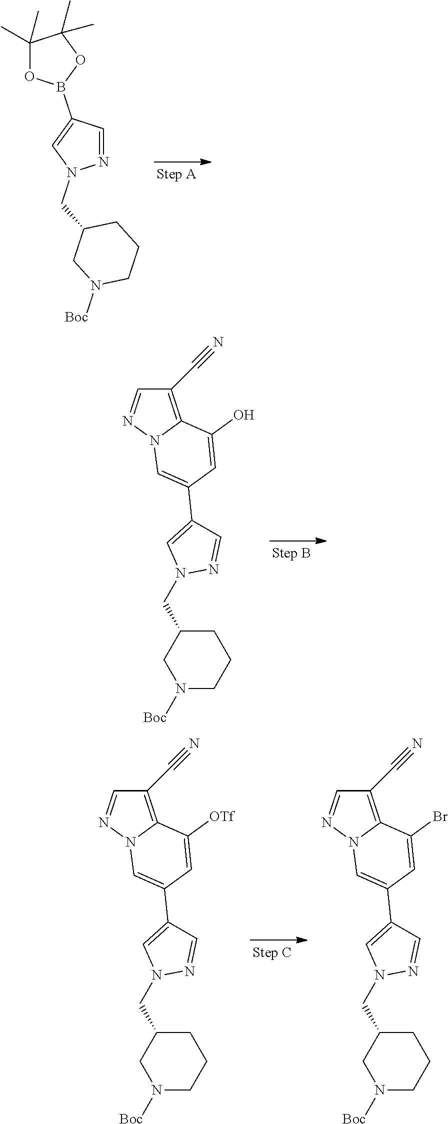

Step A. t-Butyl (3R)-3-[[4-(3-cyano-4-hydroxy-pyrazolo[1,5-a]pyridin-6-yl)pyrazol-1-yl]methyl]piperidine-1-carboxylate. Synthesized according to Intermediate 2, Step A, substituting tert-butyl (3R)-3-[[4-(4,4,5,5-tetramethyl-1,3,2-dioxaborolan-2-yl)pyrazol-1-yl]methyl]piperidine-1-carboxylate for tert-butyl (3S)-3-[4-(4,4,5,5-tetramethyl-1,3,2-dioxaborolan-2-yl)pyrazol-1-yl]piperidine-1-carboxylate to give tert-butyl (3R)-3-[[4-(3-cyano-4-hydroxy-pyrazolo[1,5-a]pyridin-6-yl)pyrazol-1-yl]methyl]piperidine-1-carboxylate (1.5 g, 76% yield) as a light yellow solid. LCMS (MM-ES+APCI, Pos): m/z 423.2 (M+H).

Step B. t-Butyl (3R)-3-[[4-[3-cyano-4-(trifluoromethylsulfonyloxy) pyrazolo[1,5-a]pyridin-6-yl]pyrazol-1-yl]methyl]piperidine-1-carboxylate. Synthesized according to Intermediate 2, Step B, substituting tert-butyl (3R)-3-[[4-(3-cyano-4-hydroxy-pyrazolo[1,5-a]pyridin-6-yl)pyrazol-1-yl]methyl]piperidine-1-carboxylate for tert-butyl (3S)-3-[4-(3-cyano-4-hydroxy-pyrazolo[1,5-a]pyridin-6-yl)pyrazol-1-yl]piperidine-1-carboxylate to give tert-butyl (3R)-3-[[4-[3-cyano-4-(trifluoromethylsulfonyloxy) pyrazolo[1,5-a]pyridin-6-yl]pyrazol-1-yl]methyl]piperidine-1-carboxylate (1.7 g, 93% yield) as a yellow solid. LCMS (MM-ES+APCI, Pos): m/z 555.0 (M+H).

Step C. t-Butyl (3R)-3-[[4-(4-bromo-3-cyano-pyrazolo[1,5-a]pyridin-6-yl)pyrazol-1-yl]methyl]piperidine-1-carboxylate. Synthesized according to Intermediate 2, Step C, substituting tert-butyl (3R)-3-[[4-[3-cyano-4-(trifluoromethylsulfonyloxy)pyrazolo[1,5-a]pyridin-6-yl]pyrazol-1-yl]methyl]piperidine-1-carboxylate for tert-butyl (3S)-3-[4-[3-cyano-4-(trifluoromethylsulfonyloxy)pyrazolo[1,5-a]pyridin-6-yl]pyrazol-1-yl]piperidine-1-carboxylate to give tert-butyl (3R)-3-[[4-(4-bromo-3-cyano-pyrazolo[1,5-a]pyridin-6-yl)pyrazol-1-yl]methyl]piperidine-1-carboxylate (0.76 g, 75% yield) as a gray solid. LCMS (MM-ES+APCI, Pos): m/z 486.9 (M+H).

### Intermediate 9

### t-Butyl 4-[4-(4-bromo-3-cyano-pyrazolo[1,5-a]pyridin-6-yl)-5-methyl-pyrazol-1-yl]piperidine-1-carboxylate

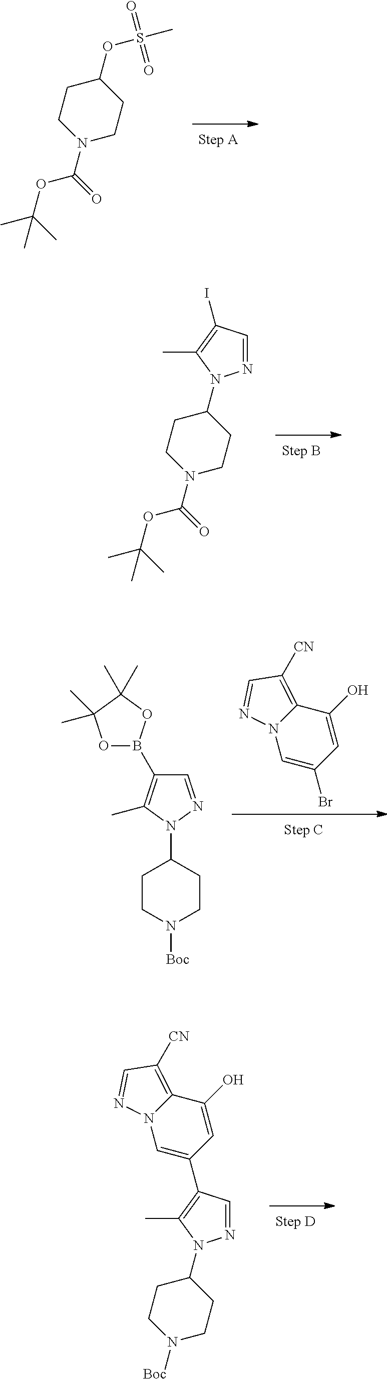 [SMILES: CC(C)(C)OC(=O)N1CCC(OS(C)(=O)=O)CC1 ; Cc1c(I)cnn1C1CCN(C(=O)OC(C)(C)C)CC1 ; Cc1c(B2OC(C)(C)C(C)(C)O2)cnn1C1CCN(C(=O)OC(C)(C)C)CC1 ; N#Cc1cnn2cc(Br)cc(O)c12 ; Cc1c(-c2cc(O)c3c(C#N)cnn3c2)cnn1C1CCN(C(=O)OC(C)(C)C)CC1]

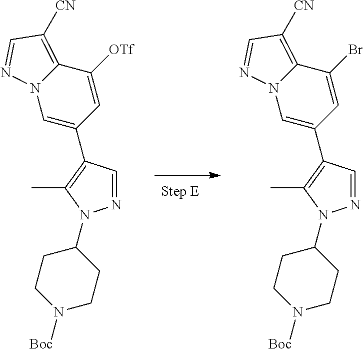 [SMILES: Cc1c(-c2cc(OS(=O)(=O)C(F)(F)F)c3c(C#N)cnn3c2)cnn1C1CCN(C(=O)OC(C)(C)C)CC1 ; Cc1c(-c2cc(Br)c3c(C#N)cnn3c2)cnn1C1CCN(C(=O)OC(C)(C)C)CC1]

Step A. t-Butyl 4-(4-iodo-5-methyl-pyrazol-1-yl) piperidine-1-carboxylate. To a round-bottom flask equipped with a magnetic stir bar and a reflux condenser was added 4-iodo-5-methyl-1H-pyrazole (10 g, 48 mmol), tert-butyl 4-methylsulfonyloxypiperidine-1-carboxylate (20 g, 72 mmol) and cesium carbonate (31 g, 96 mmol) followed by the addition of N, N-dimethylformamide (300 mL). The mixture was stirred at 90° C. for 4.5 hr. Water (600 mL) was added to the reaction and the mixture was transferred to a separatory funnel. The aqueous layer mixture was extracted with ethyl acetate (200 mL×3). The combined organic layers were washed with brine (800 mL), dried over anhydrous sodium sulfate, filtered, concentrated under reduced pressure and purified by silica gel column chromatography (0-60% EtOAc/petroleum ether) to give tert-butyl 4-(4-iodo-5-methyl-pyrazol-1-yl) piperidine-1-carboxylate (4.4 g, 23% yield) as a white solid.

Step B. t-Butyl 4-[5-methyl-4-(4, 4, 5, 5-tetramethyl-1, 3, 2-dioxaborolan-2-yl) pyrazol-1-yl]piperidine-1-carboxylate. To a 250 mL round-bottom flask equipped with a magnetic stir bar was added tert-butyl 4-(4-iodo-5-methyl-pyrazol-1-yl) piperidine-1-carboxylate (4.4 g, 11.3 mmol) followed by the addition of THF (120 mL). Isopropylmagnesium chloride (2 M, 23 mL, 45 mmol) was added to the mixture and stirred for 1 h at 25° C. 2-methoxy-4, 4, 5, 5 tetramethyl-1, 3, 2-dioxaborolane (8.9 g, 56 mmol) was added to the mixture and stirred at 25° C. for 3 h. Water (300 mL) was added to the reaction and the mixture was transferred to a separatory funnel. The aqueous mixture was extracted with ethyl acetate (3×100 mL). The combined organic layers were washed with brine (400 mL×3), dried over anhydrous sodium sulfate, filtered, concentrated under reduced pressure and purified by silica gel column chromatography (0-10% EtOAc/petroleum ether) to give tert-butyl 4-[5-methyl-4-(4, 4, 5, 5-tetramethyl-1, 3, 2-dioxaborolan-2-yl) pyrazol-1-yl] piperidine-1-carboxylate (1.9 g, 40% yield) as a white solid. LCMS (MM-ES+APCI, Pos): m/z 392.4 (M+H).

Step C. t-Butyl 4-[4-(3-cyano-4-hydroxy-pyrazolo[1,5-a]pyridin-6-yl)-5-methyl-pyrazol-1-yl]piperidine-1-carboxylate. Synthesized according to Intermediate 2, Step A, substituting tert-butyl 4-[5-methyl-4-(4, 4, 5, 5-tetramethyl-1, 3, 2-dioxaborolan-2-yl) pyrazol-1-yl] piperidine-1-carboxylate for tert-butyl (3S)-3-[4-(4,4,5,5-tetramethyl-1,3,2-dioxaborolan-2-yl)pyrazol-1-yl]piperidine-1-carboxylate to give tert-butyl 4-[4-(3-cyano-4-hydroxy-pyrazolo[1,5-a]pyridin-6-yl)-5-methyl-pyrazol-1-yl]piperidine-1-carboxylate (10 g, 80% yield) as a white solid. LCMS (MM-ES+APCI, Pos): m/z 367.0 (M-tBu+H).

Step D. t-Butyl 4-[4-[3-cyano-4-(trifluoromethylsulfonyloxy)pyrazolo[1,5-a]pyridin-6-yl]-5-methyl-pyrazol-1-yl]piperidine-1-carboxylate. Synthesized according to Intermediate 2, Step B, substituting tert-butyl 4-[4-(3-cyano-4-hydroxy-pyrazolo[1,5-a]pyridin-6-yl)-5-methyl-pyrazol-1-yl]piperidine-1-carboxylate for tert-butyl (3S)-3-[4-(3-cyano-4-hydroxy-pyrazolo[1,5-a]pyridin-6-yl)pyrazol-1-yl]piperidine-1-carboxylate to give tert-butyl 4-[4-[3-cyano-4-(trifluoromethylsulfonyloxy)pyrazolo[1,5-a]pyridin-6-yl]-5-methyl-pyrazol-1-yl] piperidine-1-carboxylate (11 g, 85% yield) as a white solid. LCMS (MM-ES+APCI, Pos): m/z 577.1 (M+H).

Step E. t-Butyl 4-[4-(4-bromo-3-cyano-pyrazolo[1,5-a]pyridin-6-yl)-5-methyl-pyrazol-1-yl]piperidine-1-carboxylate. Synthesized according to Intermediate 2, Step C, substituting tert-butyl 4-[4-[3-cyano-4-(trifluoromethylsulfonyloxy)pyrazolo[1,5-a]pyridin-6-yl]-5-methyl-pyrazol-1-yl] piperidine-1-carboxylate for tert-butyl (3S)-3-[4-[3-cyano-4-(trifluoromethylsulfonyloxy)pyrazolo[1,5-a]pyridin-6-yl]pyrazol-1-yl]piperidine-1-carboxylate to give tert-butyl 4-[4-(4-bromo-3-cyano-pyrazolo[1,5-a]pyridin-6-yl)-5-methyl-pyrazol-1-yl]piperidine-1-carboxylate (11 g, 82% yield) as a white solid. LCMS (MM-ES+APCI, Pos): m/z 430.8 (M+H).

### Intermediate 10

### t-Butyl (3R)-3-[4-(4-bromo-3-cyano-pyrazolo[1,5-a]pyridin-6-yl)-5-methyl-pyrazol-1-yl]pyrrolidine-1-carboxylate

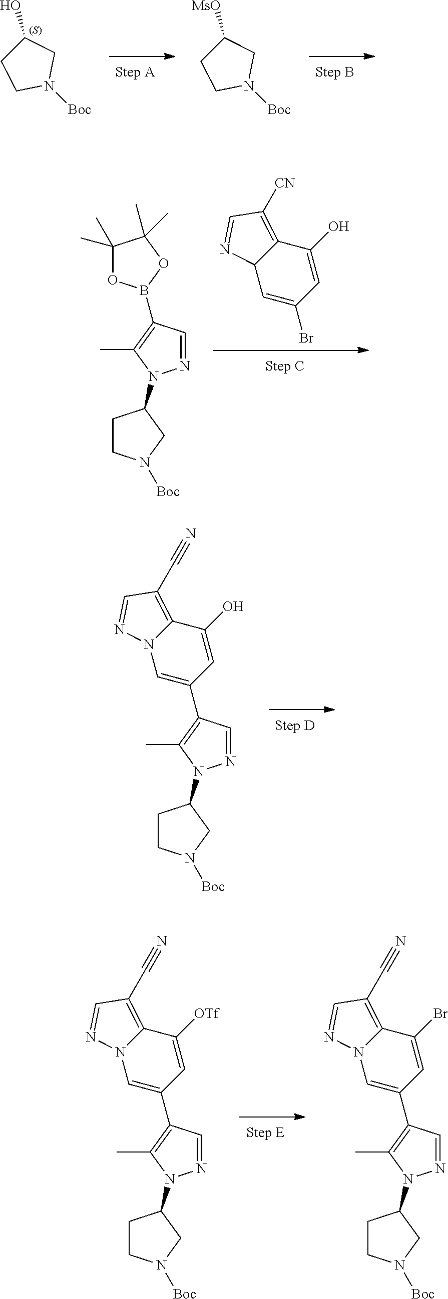

Step A. 1-Butyl (3S)-3-methylsulfonyloxypyrrolidine-1-carboxylate. To a 50 mL round-bottom equipped with a magnetic stir bar were added tert-butyl (3S)-3-hydroxypyrrolidine-1-carboxylate (3.0 g, 16.0 mmol) and triethylamine (4.5 mL, 32 mmol) followed by the addition of DCM (10 mL). The solution was cooled to 0° C. and a solution of methanesulfonic anhydride (3.4 g, 19 mmol) in DCM (10 mL) was added dropwise. Water (20 mL) was added to the reaction and the mixture was transferred to a separatory funnel. The aqueous layer mixture was extracted with DCM (20 mL×5). The combined organic layers were washed with brine (20 mL), dried over anhydrous sodium sulfate, filtered, and concentrated under reduced pressure affording tert-butyl (3S)-3-methylsulfonyloxypyrrolidine-1-carboxylate (3.5 g, 82% yield) as a yellow oil.

Step B. t-Butyl (3R)-3-[5-methyl-4-(4,4,5,5-tetramethyl-1,3, 2-dioxaborolan-2-yl)pyrazol-1-yl]pyrrolidine-1-carboxylate. To a 100 mL round-bottom flask equipped with a magnetic stir bar and a reflux condenser were added tert-butyl (3S)-3-methylsulfonyloxypyrrolidine-1-carboxylate (2.4 g, 9.1 mmol) and Cs₂CO₃ (6.0 g, 18 mmol) followed by the addition of DMF (30 mL). 5-methyl-4-(4,4,5,5-tetramethyl-1,3,2-dioxaborolan-2-yl)-1H-pyrazole (1.9 g, 9.1 mmol) was added to the mixture at 25° C. The mixture was heated to 90° C. and stirred for 2 h. Saturated ammonium chloride (20 mL) was added to the reaction and the mixture was transferred to a separatory funnel. The aqueous layer mixture was extracted with ethyl acetate (20 mL×3). The combined organic layers were washed with brine (20 mL), dried over anhydrous sodium sulfate, filtered, concentrated under reduced pressure and purified by silica gel column chromatography (28-30% ethyl acetate/petroleum ether) to give tert-butyl (3R)-3-[5-methyl-4-(4,4,5,5-tetramethyl-1,3, 2-dioxaborolan-2-yl)pyrazol-1-yl]pyrrolidine-1-carboxylate (0.48 g, 12% yield) as a white solid. LCMS (MM-ES+APCI, Pos): m/z 378.2 (M+H).

Step C. t-Butyl (3R)-3-[4-(3-cyano-4-hydroxy-pyrazolo[1,5-a]pyridin-6-yl)-5-methyl-pyrazol-1-yl]pyrrolidine-1-carboxylate. Synthesized according to Intermediate 2, Step A, substituting tert-butyl (3R)-3-[5-methyl-4-(4,4,5,5-tetramethyl-1,3,2-dioxaborolan-2-yl)pyrazol-1-yl]pyrrolidine-1-carboxylate for tert-butyl (3S)-3-[4-(4,4,5,5-tetramethyl-1,3,2-dioxaborolan-2-yl)pyrazol-1-yl]piperidine-1-carboxylate to give tert-butyl (3R)-3-[4-(3-cyano-4-hydroxy-pyrazolo[1,5-a]pyridin-6-yl)-5-methyl-pyrazol-1-yl]pyrrolidine-1-carboxylate (0.42 g, 92% yield) as a yellow solid. LCMS (MM-ES+APCI, Pos): m/z 352.9 (M+H).

Step D. 1-Butyl (3R)-3-[4-[3-cyano-4-(trifluoromethylsulfonyloxy) pyrazolo[1,5-a]pyridin-6-yl]-5-methyl-pyrazol-1-yl]pyrrolidine-1-carboxylate. Synthesized according to Intermediate 2, Step B, substituting tert-butyl (3R)-3-[4-(3-cyano-4-hydroxy-pyrazolo[1,5-a]pyridin-6-yl)-5-methyl-pyrazol-1-yl]pyrrolidine-1-carboxylate for tert-butyl (3S)-3-[4-(3-cyano-4-hydroxy-pyrazolo[1,5-a]pyridin-6-yl)pyrazol-1-yl]piperidine-1-carboxylate to give tert-butyl (3R)-3-[4-[3-cyano-4-(trifluoromethylsulfonyloxy) pyrazolo[1,5-a]pyridin-6-yl]-5-methyl-pyrazol-1-yl]pyrrolidine-1-carboxylate (0.42 g, 74% yield) as a yellow gum. LCMS (MM-ES+APCI, Pos): m/z 484.9 (M+H).

Step E. t-Butyl (3R)-3-[4-(4-bromo-3-cyano-pyrazolo[1,5-a]pyridin-6-yl)-5-methyl-pyrazol-1-yl]pyrrolidine-1-carboxylate. Synthesized according to Intermediate 2, Step C, substituting tert-butyl (3R)-3-[4-[3-cyano-4-(trifluoromethylsulfonyloxy)pyrazolo[1,5-a]pyridin-6-yl]-5-methyl-pyrazol-1-yl]pyrrolidine-1-carboxylate for tert-butyl (3S)-3-[4-[3-cyano-4-(trifluoromethylsulfonyloxy)pyrazolo[1,5-a]pyridin-6-yl]pyrazol-1-yl]piperidine-1-carboxylate to give tert-butyl (3R)-3-[4-(4-bromo-3-cyano-pyrazolo[1,5-a]pyridin-6-yl)-5-methyl-pyrazol-1-yl]pyrrolidine-1-carboxylate (0.44 g, 95% yield) as a yellow oil. LCMS (MM-ES+APCI, Pos): m/z 416.7 (M+H).

### Intermediate 11 t-Butyl N-[4-[4-(4-bromo-3-cyano-pyrazolo[1,5-a]pyridin-6-yl)-5-methyl-pyrazol-1-yl]cyclohexyl]-N-methyl-carbamate

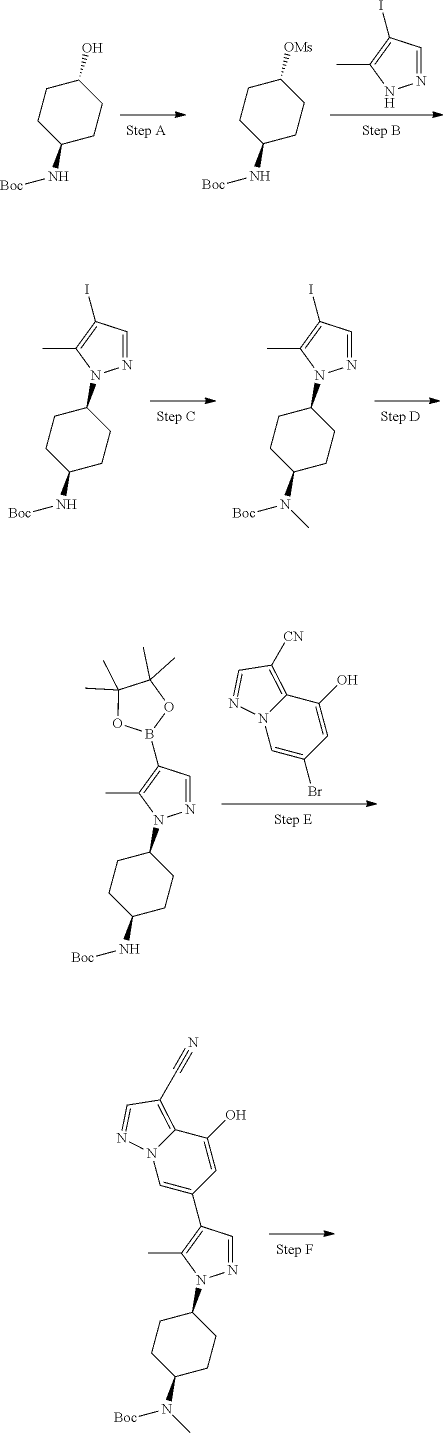

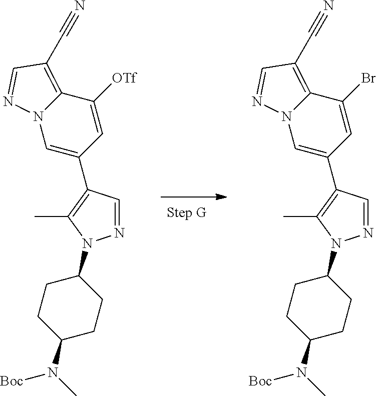 [SMILES: Cc1c(-c2cc(OS(=O)(=O)C(F)(F)F)c3c(C#N)cnn3c2)cnn1[C@H]1CC[C@@H](N(C)C(=O)OC(C)(C)C)CC1 ; Cc1c(-c2cc(Br)c3c(C#N)cnn3c2)cnn1[C@H]1CC[C@@H](N(C)C(=O)OC(C)(C)C)CC1]

Step A. 4-(t-Butoxycarbonylamino)cyclohexyl] methanesulfonate. Synthesized according to Intermediate 10, Step A substituting trans tert-butyl N-(4-hydroxycyclohexyl)carbamate for tert-butyl (3S)-3-hydroxypyrrolidine-1-carboxylate to give 4-(tert-butoxycarbonylamino)cyclohexyl] methanesulfonate (13 g, 9% yield) as a yellow solid.

Step B. t-Butyl N-[4-(4-iodo-5-methyl-pyrazol-1-yl)cyclohexyl]carbamate. Synthesized according to Intermediate 9, Step A substituting trans [4-(t-butoxycarbonylamino)cyclohexyl] methanesulfonate for tert-butyl 4-methylsulfonyloxypiperidine-1-carboxylate to give tert-butyl N-[4-(4-iodo-5-methyl-pyrazol-1-yl)cyclohexyl]carbamate (4.2 g, 31% yield) as a colorless oil.

Step C. t-Butyl N-[4-(4-iodo-5-methyl-pyrazol-1-yl)cyclohexyl]-N-methyl-carbamate. To a 100 mL round-bottom flask equipped with a magnetic stir bar was added tert-butyl N-[4-(4-iodo-5-methyl-pyrazol-1-yl)cyclohexyl]carbamate (1.2 g, 3.0 mmol) followed by the addition of THF (20 mL). The solution was cooled to 0° C. and treated with NaH (60%, 0.24 g, 5.9 mmol). After stirring at 0° C. for 1 h, CH₃I (0.55 mL, 8.9 mmol) was added, the reaction warmed to 25° C. and stirred for 12 h. Water (30 mL) was added to the reaction and the mixture was transferred to a separatory funnel. The mixture was extracted with ethyl acetate (30 mL×3). The combined organic layer was washed with brine (30 mL), dried over anhydrous sodium sulfate, filtered, concentrated under reduced pressure and purified by silica gel column chromatography (0-70% EtOAc/petroleum ether) to give tert-butyl N-[4-(4-iodo-5-methyl-pyrazol-1-yl)cyclohexyl]-N-methyl-carbamate (1.2 g, 97% yield) as a yellow solid.

Step D. t-Butyl N-methyl-N-[4-[5-methyl-4-(4,4,5,5-tetramethyl-1,3,2-dioxaborolan-2-yl)pyrazol-1-yl]cyclohexyl]carbamate. Synthesized according to Intermediate 9, Step A substituting cis tert-butyl N-[4-(4-iodo-5-methyl-pyrazol-1-yl)cyclohexyl]-N-methyl-carbamate for tert-butyl 4-(4-iodo-5-methyl-pyrazol-1-yl) piperidine-1-carboxylate to give tert-butyl N-methyl-N-[4-[5-methyl-4-(4,4,5,5-tetramethyl-1,3,2-dioxaborolan-2-yl)pyrazol-1-yl]cyclohexyl]carbamate (1.1 g, 85% yield) as a white solid.

Step E. t-Butyl N-[4-[4-(3-cyano-4-hydroxy-pyrazolo[1,5-a]pyridin-6-yl)-5-methyl-pyrazol-1-yl]cyclohexyl]-N-methyl-carbamate. Synthesized according to Intermediate 2, Step A, substituting cis tert-butyl N-methyl-N-[4-[5-methyl-4-(4,4,5,5-tetramethyl-1,3,2-dioxaborolan-2-yl)pyrazol-1-yl]cyclohexyl]carbamate for tert-butyl (3S)-3-[4-(4,4,5,5-tetramethyl-1,3,2-dioxaborolan-2-yl)pyrazol-1-yl]piperidine-1-carboxylate to give tert-butyl N-[4-[4-(3-cyano-4-hydroxy-pyrazolo[1,5-a]pyridin-6-yl)-5-methyl-pyrazol-1-yl]cyclohexyl]-N-methyl-carbamate (0.60 g, 53% yield) as a brown solid.

Step F. [6-[1-[4-[t-Butoxycarbonyl(methyl)amino]cyclohexyl]-5-methyl-pyrazol-4-yl]-3-cyano-pyrazolo[1,5-a]pyridin-4-yl] trifluoromethanesulfonate. Synthesized according to Intermediate 2, Step B, substituting cis tert-butyl N-[4-[4-(3-cyano-4-hydroxypyrazolo[1,5-a]pyridin-6-yl)-5-methyl-pyrazol-1-yl]cyclohexyl]-N-methyl-carbamate for tert-butyl (3S)-3-[4-(3-cyano-4-hydroxy-pyrazolo[1,5-a]pyridin-6-yl)pyrazol-1-yl]piperidine-1-carboxylate to give [6-[1-[4-[tert-butoxycarbonyl(methyl)amino]cyclohexyl]-5-methyl-pyrazol-4-yl]-3-cyano-pyrazolo[1,5-a]pyridin-4-yl] trifluoromethanesulfonate (0.60 g, 81% yield) as a yellow oil. LCMS (MM-ES+APCI, Pos): m/z 583.4 (M+H).

Step G. t-Butyl N-[4-[4-(4-bromo-3-cyano-pyrazolo[1,5-a]pyridin-6-yl)-5-methyl-pyrazol-1-yl]cyclohexyl]-N-methyl-carbamate. Synthesized according to Intermediate 2, Step C, substituting cis [6-[1-[4-[tert-butoxycarbonyl(methyl)amino]cyclohexyl]-5-methyl-pyrazol-4-yl]-3-cyano-pyrazolo[1,5-a]pyridin-4-yl] trifluoromethanesulfonate for tert-butyl (3S)-3-[4-[3-cyano-4-(trifluoromethylsulfonyloxy)pyrazolo[1,5-a]pyridin-6-yl]pyrazol-1-yl]piperidine-1-carboxylate to give tert-butyl N-[4-[4-(4-bromo-3-cyano-pyrazolo[1,5-a]pyridin-6-yl)-5-methyl-pyrazol-1-yl]cyclohexyl]-N-methyl-carbamate (0.34 g, 68% yield) was obtained as a white solid. LCMS (MM-ES+APCI, Pos): m/z 515.0 (M+H).

### Intermediate 12

### 6-Bromo-4-(2-cyanophenyl)sulfanyl-pyrazolo[1,5-a]pyridine-3-carbonitrile

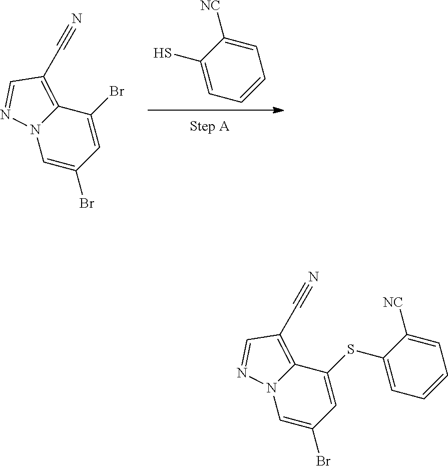 [SMILES: N#Cc1cnn2cc(Br)cc(Br)c12 ; N#Cc1ccccc1S ; N#Cc1ccccc1Sc1cc(Br)cn2ncc(C#N)c12]

Step A. 6-Bromo-4-(2-cyanophenyl)sulfanyl-pyrazolo[1,5-a]pyridine-3-carbonitrile. To a 8 mL vial equipped with a magnetic stir bar was added 4,6-dibromopyrazolo[1,5-a]pyridine-3-carbonitrile (0.50 g, 1.6 mmol) followed by the addition of DMF (4 mL). Methanesulfonato{[4-(N,N-dimethylamino)phenyl]di-t-butylphosphino} (2′-amino-1,1′-biphenyl-2-yl)palladium(II) (10 mg, 0.016 mmol), K₂CO₃ (0.44 g, 3.2 mmol) and 2-sulfanylbenzonitrile (0.17 g, 1.3 mmol) were added into the mixture at 25° C. The flask was purged with nitrogen and stirred at 100° C. for 2 h. Water (10 mL) was added to the reaction and the mixture was transferred to a separatory funnel and the aqueous layer mixture was extracted with ethyl acetate (15 mL×3). The combined organic layers were washed with brine (10 mL), dried over anhydrous sodium sulfate, filtered, concentrated under reduced pressure and purified by silica gel column chromatography (20-40% ethyl acetate/petroleum ether) to give 6-bromo-4-(2-cyanophenyl)sulfanyl-pyrazolo[1,5-a]pyridine-3-carbonitrile (0.85 g, 72% yield) as a light yellow solid. LCMS (MM-ES+APCI, Pos): m/z 355.0 (M+H).

### Intermediate 13

### 6-Bromo-4-[(3-fluoro-2-pyridyl)sulfanyl]pyrazolo[1,5-a]pyridine-3-carbonitrile

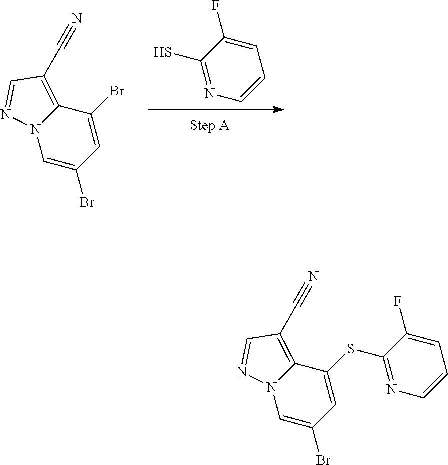 [SMILES: N#Cc1cnn2cc(Br)cc(Br)c12 ; Fc1cccnc1S ; N#Cc1cnn2cc(Br)cc(Sc3ncccc3F)c12]

Step A. 6-Bromo-4-[(3-fluoro-2-pyridyl)sulfanyl]pyrazolo[1,5-a]pyridine-3-carbonitrile. To a 40 mL vial equipped with a magnetic stir bar was added 4,6-dibromopyrazolo[1,5-a]pyridine-3-carbonitrile (2.5 g, 8.3 mmol), xantphos-Pd-G₃ (0.47 g, 0.49 mmol) and K₂CO₃ (2.5 g, 18 mmol) followed by the addition of DMF (20 mL). 3-fluoropyridine-2-thiol (1.2 g, 9.3 mmol) was added to the mixture at 25° C. The vial was purged with nitrogen for 2 mins. The mixture was heated to 90° C. and stirred for 1 h. Water (50 mL) was added to the reaction and the mixture was transferred to a separatory funnel. The aqueous layer was extracted with ethyl acetate (50 mL×3). The combined organic layers were washed with brine (50 mL), dried over anhydrous sodium sulfate, filtered, and purified by slurry in MeOH (20 mL) at 25° ° C. for 0.5 h. After filtration and drying under vacuum, 6-bromo-4-[(3-fluoro-2-pyridyl)sulfanyl]pyrazolo[1,5-a]pyridine-3-carbonitrile (2.2 g, 71% yield) was obtained as a yellow solid. LCMS (MM-ES+APCI, Pos): m/z 349.3 (M+H).

### Example 1

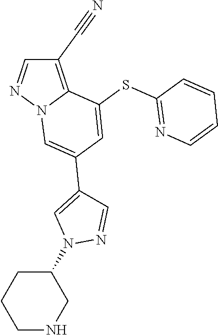 [SMILES: N#Cc1cnn2cc(-c3cnn([C@H]4CCCNC4)c3)cc(Sc3ccccn3)c12]

### 6-[1-[(3S)-3-Piperidyl]pyrazol-4-yl]-4-(2-pyridylsulfanyl) pyrazolo[1,5-a]pyridine-3-carbonitrile (1)

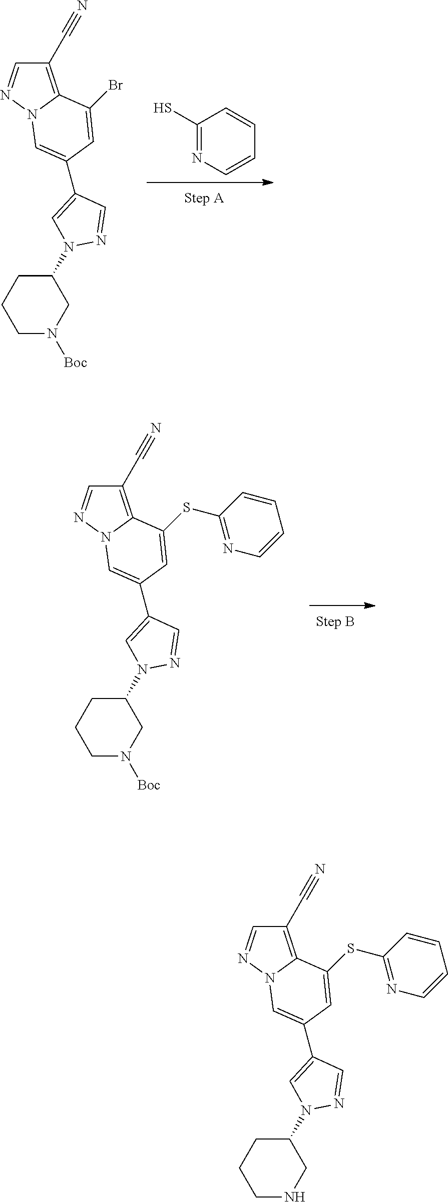 [SMILES: CC(C)(C)OC(=O)N1CCC[C@H](n2cc(-c3cc(Br)c4c(C#N)cnn4c3)cn2)C1 ; Sc1ccccn1 ; CC(C)(C)OC(=O)N1CCC[C@H](n2cc(-c3cc(Sc4ccccn4)c4c(C#N)cnn4c3)cn2)C1 ; N#Cc1cnn2cc(-c3cnn([C@H]4CCCNC4)c3)cc(Sc3ccccn3)c12]

Step A. t-Butyl (3S)-3-[4-[3-cyano-4-(2-pyridylsulfanyl) pyrazolo[1,5-a]pyridin-6-yl]pyrazol-1-yl]piperidine-1-carboxylate. To a 40 mL vial equipped with a magnetic stir bar was added tert-butyl (3S)-3-[4-(4-bromo-3-cyano-pyrazolo[1,5-a]pyridin-6-yl)pyrazol-1-yl]piperidine-1-carboxylate (0.30 g, 0.63 mmol), pyridine-2-thiol (0.11 g, 0.95 mmol) followed by the addition of DMF (5 mL). Cs₂CO₃ (0.41 g, 1.3 mmol) was added to the mixture and stirred at 120° C. for 1 h. Water (10 mL) was added to the reaction and the mixture was transferred to a separatory funnel. The aqueous layer was extracted with ethyl acetate (10 mL×3). The combined organic layers were washed with brine (35 mL), dried over anhydrous sodium sulfate, filtered, concentrated under reduced pressure and purified by silica gel column chromatography (50-75% EtOAc/petroleum ether) to give tert-butyl (3S)-3-[4-[3-cyano-4-(2-pyridylsulfanyl)pyrazolo[1,5-a]pyridin-6-yl]pyrazol-1-yl]piperidine-1-carboxylate (0.17 g, 52% yield) as a yellow solid. LCMS (MM-ES+APCI, Pos): m/z 502.1 (M+H).

Step B. 6-[1-[(3S)-3-Piperidyl]pyrazol-4-yl]-4-(2-pyridylsulfanyl) pyrazolo[1,5-a]pyridine-3-carbonitrile. To a 50 mL round-bottom flask equipped with a magnetic stir bar was added tert-butyl (3S)-3-[4-[3-cyano-4-(2-pyridylsulfanyl)pyrazolo[1,5-a]pyridin-6-yl]pyrazol-1-yl]piperidine-1-carboxylate (0.12 g, 0.23 mmol) followed by the addition of DCM (2 mL). HCl/dioxane (4 M, 2 mL) was added into the mixture at 25° C. and stirred for 0.5 h. The mixture was concentrated under reduced pressure and purified by preparative HPLC: (16%-46% acetonitrile/0.05% aqueous ammonia hydroxide+10 mM NH₄HCO₃). After lyophilization, 6-[1-[(3S)-3-piperidyl]pyrazol-4-yl]-4-(2-pyridylsulfanyl) pyrazolo[1,5-a]pyridine-3-carbonitrile (47 mg, 49% yield) was obtained as a white solid. LCMS (MM-ES+APCI, Pos): m/z 402.2 (M+H).

### Example 2

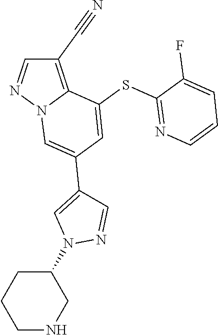 [SMILES: N#Cc1cnn2cc(-c3cnn([C@H]4CCCNC4)c3)cc(Sc3ncccc3F)c12]

### 4-[(3-fluoro-2-pyridyl)sulfanyl]-6-[1-[(3S)-3-piperidyl]pyrazol-4-yl]pyrazolo[1,5-a]pyridine-3-carbonitrile (2)

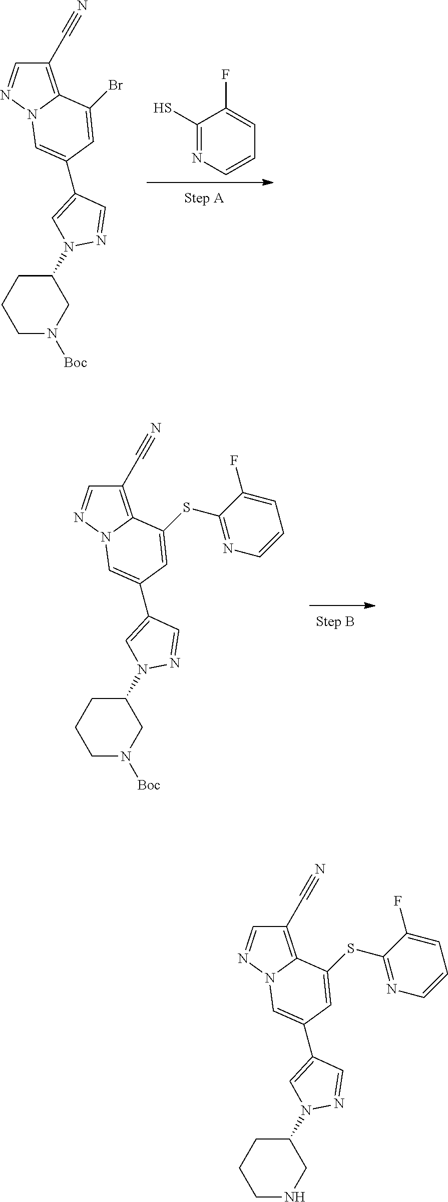 [SMILES: CC(C)(C)OC(=O)N1CCC[C@H](n2cc(-c3cc(Br)c4c(C#N)cnn4c3)cn2)C1 ; Fc1cccnc1S ; CC(C)(C)OC(=O)N1CCC[C@H](n2cc(-c3cc(Sc4ncccc4F)c4c(C#N)cnn4c3)cn2)C1 ; N#Cc1cnn2cc(-c3cnn([C@H]4CCCNC4)c3)cc(Sc3ncccc3F)c12]

Step A. t-Butyl (3S)-3-[4-[3-cyano-4-[(3-fluoro-2-pyridyl)sulfanyl]pyrazolo[1,5-a]pyridin-6-yl]pyrazol-1-yl]piperidine-1-carboxylate. Synthesized according to Example 1, Step A, substituting 3-fluoropyridine-2-thiol for pyridine-2 to give tert-butyl (3S)-3-[4-[3-cyano-4-[(3-fluoro-2-pyridyl)sulfanyl]pyrazolo[1,5-a]pyridin-6-yl]pyrazol-1-yl]piperidine-1-carboxylate (0.12 g, 41% yield) obtained as a yellow solid. LCMS (MM-ES+APCI, Pos): m/z 520.2 (M+H).

Step B. 4-[(3-Fluoro-2-pyridyl)sulfanyl]-6-[1-[(3S)-3-piperidyl]pyrazol-4-yl]pyrazolo[1,5-a]pyridine-3-carbonitrile. Synthesized according to Example 1, Step B, substituting tert-butyl (3S)-3-[4-[3-cyano-4-[(3-fluoro-2-pyridyl)sulfanyl]pyrazolo[1,5-a]pyridin-6-yl]pyrazol-1-yl]piperidine-1-carboxylate for tert-butyl (3S)-3-[4-[3-cyano-4-(2-pyridylsulfanyl)pyrazolo[1,5-a]pyridin-6-yl]pyrazol-1-yl]piperidine-1-carboxylate to give 4-[(3-fluoro-2-pyridyl)sulfanyl]-6-[1-[(3S)-3-piperidyl]pyrazol-4-yl]pyrazolo[1,5-a]pyridine-3-carbonitrile (24 mg, 48% yield) as a white solid. LCMS (MM-ES+APCI, Pos): m/z 420.3 (M+H).

### Example 3

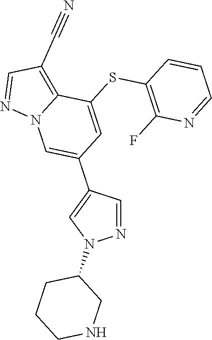 [SMILES: N#Cc1cnn2cc(-c3cnn([C@H]4CCCNC4)c3)cc(Sc3cccnc3F)c12]

### 4-[(2-Fluoro-3-pyridyl) sulfanyl]-6-[1-[(3S)-3-piperidyl] pyrazol-4-yl] pyrazolo[1,5-a] pyridine-3-carbonitrile (3)

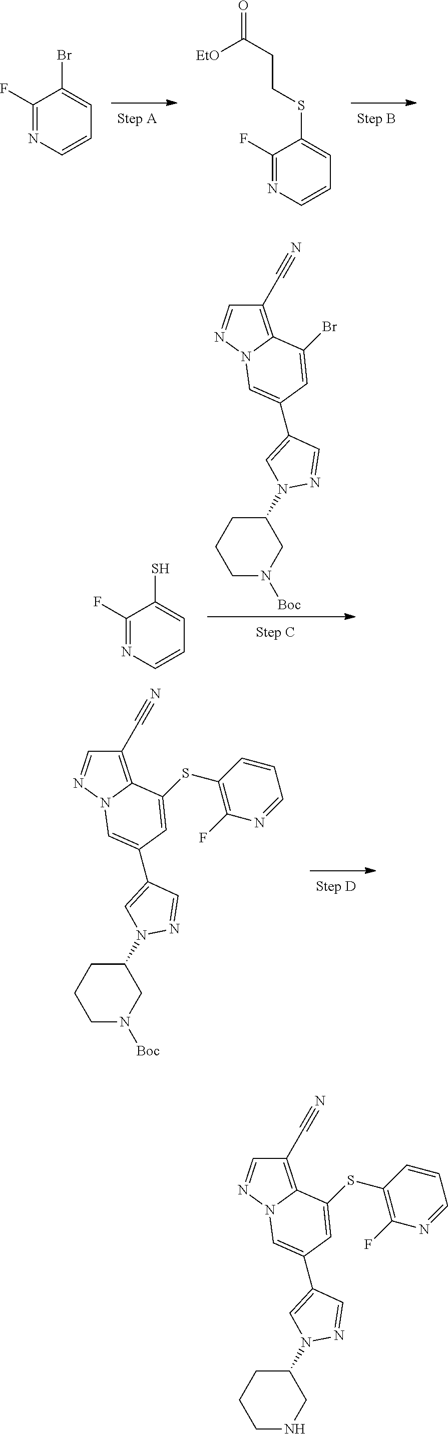

Step A. Ethyl 3-[(2-fluoro-3-pyridyl) sulfanyl] propanoate. To a 40 mL vial equipped with a magnetic stir bar was added 3-bromo-2-fluoro-pyridine (1.0 g, 5.7 mmol), ethyl 3-sulfanylpropanoate (0.76 g, 5.7 mmol), Pd₂(dba)₃ (0.16 g, 0.17 mmol), xantphos (99 mg, 0.17 mmol) and K₃PO₄ (1.5 g, 6.8 mmol) followed by the addition of dioxane (10 mL). The vial was purged with nitrogen for 2 minutes and stirred at 100° C. under an atmosphere of nitrogen for 3 h. Water (40 mL) was added to the reaction and the mixture was transferred to a separatory funnel. The aqueous layer was extracted with ethyl acetate (15 mL×3). The combined organic layers were washed with brine (50 mL), dried over anhydrous sodium sulfate, filtered, concentrated under reduced pressure and purified by silica gel column chromatography (0-40% EtOAc/petroleum ether) to give ethyl 3-[(2-fluoro-3-pyridyl) sulfanyl] propanoate (1.0 g, 79% yield) as a yellow oil. LCMS (MM-ES+APCI, Pos): m/z 230.2 (M+H).

Step B. 2-Fluoropyridine-3-thiol. To a 40 ml vial equipped with a magnetic stir bar was added ethyl 3-[(2-fluoro-3-pyridyl) sulfanyl] propanoate (0.20 g, 0.87 mmol) followed by the addition of DMF (10 mL). The solution was cooled to 0° C. and t-BuOK (1 M, 1.1 mL) was added dropwise. The mixture was stirred at 0° C. for 1 hr. The mixture was concentrated under reduced pressure affording the crude product 2-fluoropyridine-3-thiol (0.10 g, crude) as a yellow oil, used in the next step without further purification.

Step C. t-Butyl (3S)-3-[4-[3-cyano-4-[(2-fluoro-3-pyridyl) sulfanyl] pyrazolo[1,5-a] pyridin-6-yl] pyrazol-1-yl] piperidine-1-carboxylate. To a 40 mL vial equipped with a magnetic stir bar was added tert-butyl (3S)-3-[4-(4-bromo-3-cyano-pyrazolo[1,5-a]pyridin-6-yl) pyrazol-1-yl] piperidine-1-carboxylate (0.20 g, 0.42 mmol), 2-fluoropyridine-3-thiol (0.10 g, 0.77 mmol) and Cs₂CO₃ (0.32 mg, 0.98 mmol) followed by the addition of DMF (10 mL) at 25° C. The mixture was stirred at 110° C. for 2 h. Water (30 mL) was added to the reaction and the mixture was transferred to a separatory funnel. The aqueous layer was extracted with ethyl acetate (15 mL×3). The combined organic layers were washed with brine (40 mL), dried over anhydrous sodium sulfate, filtered, concentrated under reduced pressure and purified by silica gel column chromatography (0-60% EtOAc/petroleum ether) to give tert-butyl (3S)-3-[4-[3-cyano-4-[(2-fluoro-3-pyridyl) sulfanyl] pyrazolo[1,5-a] pyridin-6-yl] pyrazol-1-yl] piperidine-1-carboxylate (0.30 g, crude) as a yellow oil. LCMS (MM-ES+APCI, Pos): m/z 420.4 (M-Boc+H).

Step D. 4-[(2-Fluoro-3-pyridyl) sulfanyl]-6-[1-[(3S)-3-piperidyl] pyrazol-4-yl] pyrazolo[1, 5-a]pyridine-3-carbonitrile. To a 40 mL vial equipped with a magnetic stir bar was added tert-butyl (3S)-3-[4-[3-cyano-4-[(2-fluoro-3-pyridyl) sulfanyl] pyrazolo[1,5-a]pyridin-6-yl] pyrazol-1-yl] piperidine-1-carboxylate (0.20 g, 0.38 mmol) followed by the addition of dichloromethane (5 mL). Hydrochloride/dioxane (4 M, 5 mL) was added into the mixture and the mixture stirred at 25° C. for 1 h. The mixture was concentrated under reduced pressure and purified by preparative HPLC: (30%-60% acetonitrile/0.04% aqueous ammonia hydroxide+10 mM NH₄HCO₃). After lyophilization, 4-[(2-fluoro-3-pyridyl) sulfanyl]-6-[1-[(3S)-3-piperidyl] pyrazol-4-yl] pyrazolo[1,5-a]pyridine-3-carbonitrile (2.9 mg, 2% yield) was obtained as an off-white solid. LCMS (MM-ES+APCI, Pos): m/z 420.2 (M+H).

### Example 4

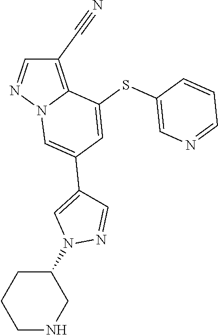 [SMILES: N#Cc1cnn2cc(-c3cnn([C@H]4CCCNC4)c3)cc(Sc3cccnc3)c12]

### 6-[1-[(3S)-3-Piperidyl] pyrazol-4-yl]-4-(3-pyridylsulfanyl)pyrazolo[1,5-a]pyridine-3-carbonitrile (4)

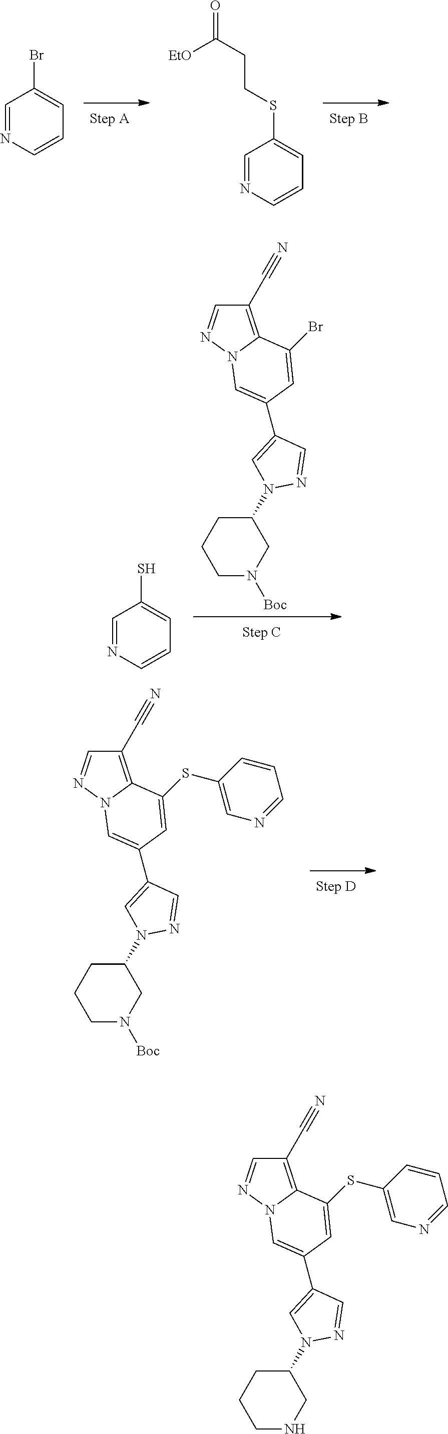

Step A. Ethyl 3-(3-pyridylsulfanyl) propanoate. Synthesized according to Example 3, Step A, substituting 3-bromopyridine for 3-bromo-2-fluoro-pyridine to give ethyl 3-(3-pyridylsulfanyl) propanoate (1.56 g, crude) obtained as yellow oil. LCMS (MM-ES+APCI, Pos): m/z 212.6 (M+H).

Step B. Pyridine-3-thiol. Synthesized according to Example 3, Step B, substituting ethyl 3-(3-pyridylsulfanyl) propanoate for ethyl 3-[(2-fluoro-3-pyridyl) sulfanyl] propanoate to give pyridine-3-thiol (0.10 g, crude) obtained as yellow oil.

Step C. t-Butyl (3S)-3-[4-[3-cyano-4-(3-pyridylsulfanyl) pyrazolo[1,5-a]pyridin-6-yl] pyrazol-1-yl] piperidine-1-carboxylate. Synthesized according to Example 3, Step C, substituting pyridine-3-thiol for 2-fluoropyridine-3-thiol to give tert-butyl (3S)-3-[4-[3-cyano-4-(3-pyridylsulfanyl) pyrazolo[1,5-a]pyridin-6-yl] pyrazol-1-yl] piperidine-1-carboxylate (0.31 g, 94% yield) obtained as a yellow oil. LCMS (MM-ES+APCI, Pos): m/z 502.3 (M+H).

Step D. 6-[1-[(3S)-3-Piperidyl] pyrazol-4-yl]-4-(3-pyridylsulfanyl)pyrazolo[1,5-a]pyridine-3-carbonitrile. Synthesized according to Example 3, Step D, substituting tert-butyl (3S)-3-[4-[3-cyano-4-(3-pyridylsulfanyl) pyrazolo[1,5-a]pyridin-6-yl] pyrazol-1-yl] piperidine-1-carboxylate for tert-butyl (3S)-3-[4-[3-cyano-4-[(2-fluoro-3-pyridyl) sulfanyl] pyrazolo[1, 5-a]pyridin-6-yl] pyrazol-1-yl] piperidine-1-carboxylate to give 6-[1-[(3S)-3-piperidyl] pyrazol-4-yl]-4-(3-pyridylsulfanyl)pyrazolo[1,5-a]pyridine-3-carbonitrile (42 mg, 27% yield) was obtained as a white solid. LCMS (MM-ES+APCI, Pos): m/z 402.2 (M+H).

### Example 5

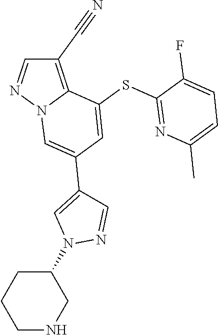 [SMILES: Cc1ccc(F)c(Sc2cc(-c3cnn([C@H]4CCCNC4)c3)cn3ncc(C#N)c23)n1]

### 4-[(3-Fluoro-6-methyl-2-pyridyl)sulfanyl]-6-[1-[(3S)-3-piperidyl]pyrazol-4-yl]pyrazolo[1,5-a]pyridine-3-carbonitrile (5)

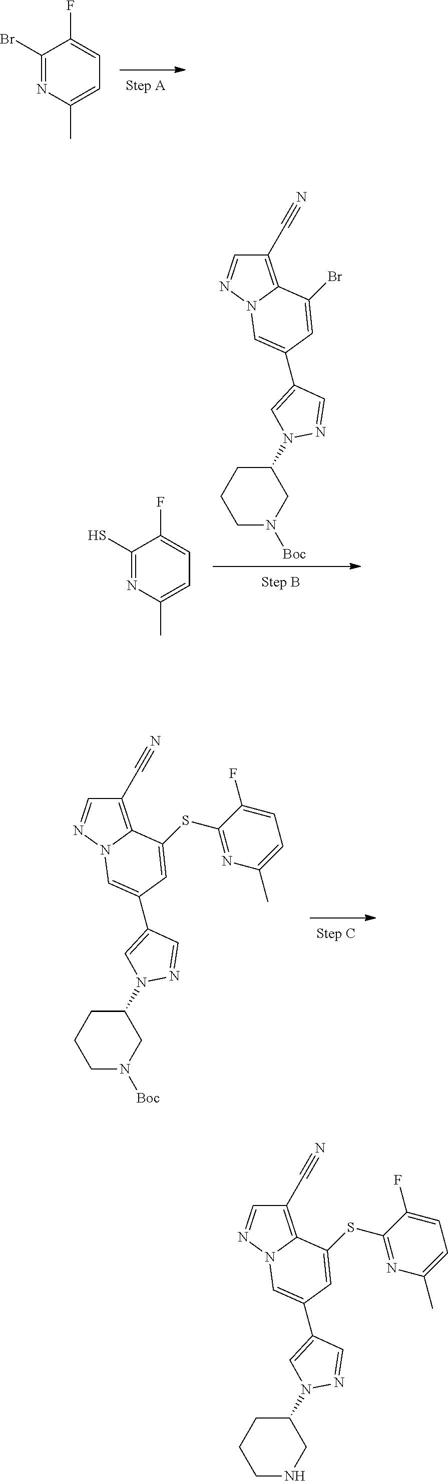

Step A. 3-Fluoro-6-methyl-pyridine-2-thiol. To a 250 mL three-necked round-bottom flask equipped with a magnetic stir bar was added 2-bromo-3-fluoro-6-methyl-pyridine (2.0 g, 10 mmol) followed by the addition of toluene (25 mL). The solution was cooled to −78° C. and purged with nitrogen three times. n-BuLi (2.5 M, 6.3 mL, 15.8 mmol) was added dropwise and mixture stirred at −78° ° C. for 1 h. Sulfur (0.64 g, 20 mmol) was added to the mixture and stirred at −78° C. for 0.5 h and at 25° C. for 1 h. Water (25 mL) was added to the reaction and the pH of mixture adjusted to 5 by using hydrochloric acid (1 M). The mixture was transferred to a separatory funnel and the aqueous layer extracted with DCM (20 mL×3). The combined organic layers were washed with brine (20 mL×3), dried over anhydrous sodium sulfate, filtered, concentrated under reduced pressure and purified by silica gel chromatography (50-100% EtOAc/petroleum ether) to give 3-fluoro-6-methyl-pyridine-2-thiol (1.0 g, 58% yield) was obtained as a yellow solid. LCMS (MM-ES+APCI, Pos): m/z 144.3 (M+H).

Step B. t-Butyl (3S)-3-[4-[3-cyano-4-[(3-fluoro-6-methyl-2-pyridyl)sulfanyl]pyrazolo[1,5-a]pyridin-6-yl]pyrazol-1-yl]piperidine-1-carboxylate. Synthesized according to Example 3, Step C, substituting 3-fluoro-6-methyl-pyridine-2-thiol for 2-fluoropyridine-3-thiol to give tert-butyl (3S)-3-[4-[3-cyano-4-[(3-fluoro-6-methyl-2-pyridyl)sulfanyl]pyrazolo[1,5-a]pyridin-6-yl]pyrazol-1-yl]piperidine-1-carboxylate (0.14 g, 79% yield) as a yellow oil. LCMS (MM-ES+APCI, Pos): m/z 534.5 (M+H).

Step C. 4-[(3-Fluoro-6-methyl-2-pyridyl)sulfanyl]-6-[1-[(3S)-3-piperidyl]pyrazol-4-yl]pyrazolo[1,5-a]pyridine-3-carbonitrile. Synthesized according to Example 3, Step D, substituting tert-butyl (3S)-3-[4-[3-cyano-4-[(3-fluoro-6-methyl-2-pyridyl)sulfanyl]pyrazolo[1,5-a]pyridin-6-yl]pyrazol-1-yl]piperidine-1-carboxylate for tert-butyl (3S)-3-[4-[3-cyano-4-[(2-fluoro-3-pyridyl) sulfanyl] pyrazolo[1,5-a]pyridin-6-yl] pyrazol-1-yl] piperidine-1-carboxylate to give 4-[(3-fluoro-6-methyl-2-pyridyl)sulfanyl]-6-[1-[(3S)-3-piperidyl]pyrazol-4-yl]pyrazolo[1,5-a]pyridine-3-carbonitrile (10 mg, 10% yield) as an off-white solid. LCMS (MM-ES+APCI, Pos): m/z 434.4 (M+H).

### Example 6

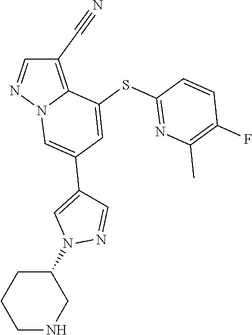 [SMILES: Cc1nc(Sc2cc(-c3cnn([C@H]4CCCNC4)c3)cn3ncc(C#N)c23)ccc1F]

### 4-[(5-Fluoro-6-methyl-2-pyridyl) sulfanyl]-6-[1-[(3S)-3-piperidyl] pyrazol-4-yl] pyrazolo[1, 5-a]pyridine-3-carbonitrile (6)

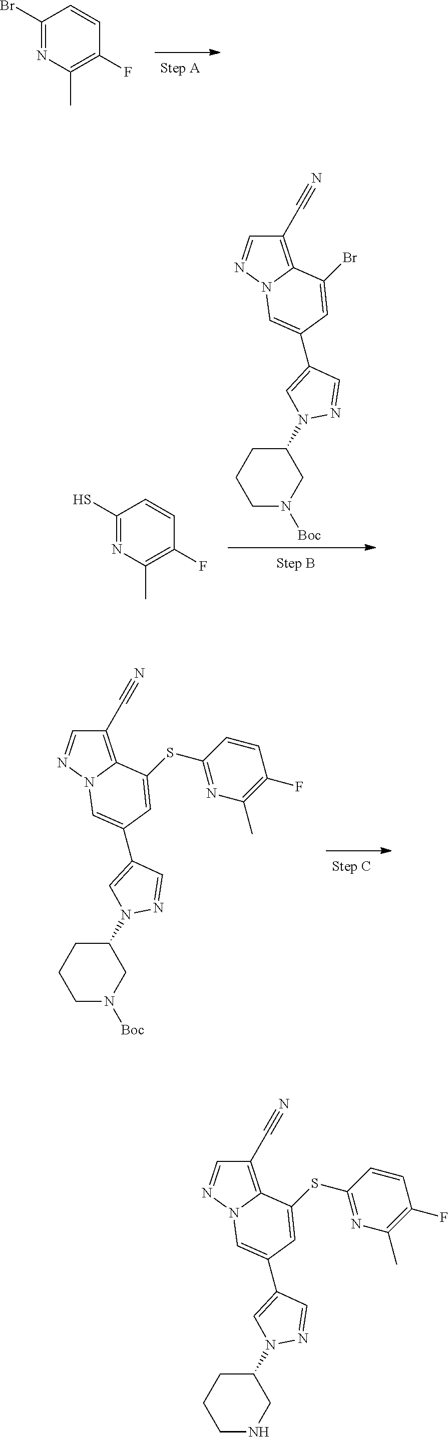

Step A. 5-Fluoro-6-methyl-pyridine-2-thiol. Synthesized according to Example 5, Step A, substituting 6-bromo-3-fluoro-2-methyl-pyridine for 2-bromo-3-fluoro-6-methyl-pyridine to give 5-fluoro-6-methyl-pyridine-2-thiol (0.29 g, 77% yield) as a yellow solid. LCMS (MM-ES+APCI, Pos): m/z 144.1 (M+H).

Step B. t-Butyl (3S)-3-[4-[3-cyano-4-[(5-fluoro-6-methyl-2-pyridyl) sulfanyl]pyrazolo[1,5-a]pyridin-6-yl] pyrazol-1-yl] piperidine-1-carboxylate. Synthesized according to Example 3, Step C, substituting 5-fluoro-6-methyl-pyridine-2-thiol for 2-fluoropyridine-3-thiol to give tert-butyl (3S)-3-[4-[3-cyano-4-[(5-fluoro-6-methyl-2-pyridyl) sulfanyl]pyrazolo[1,5-a] pyridin-6-yl] pyrazol-1-yl] piperidine-1-carboxylate (0.17 g, 97% yield) as a yellow solid. LCMS (MM-ES+APCI, Pos): m/z 534.2 (M+H).

Step C. 4-[(5-Fluoro-6-methyl-2-pyridyl) sulfanyl]-6-[1-[(3S)-3-piperidyl] pyrazol-4-yl] pyrazolo[1,5-a]pyridine-3-carbonitrile. Synthesized according to Example 3, Step D, substituting tert-butyl (3S)-3-[4-[3-cyano-4-[(5-fluoro-6-methyl-2-pyridyl)sulfanyl]pyrazolo[1,5-a]pyridin-6-yl]pyrazol-1-yl]piperidine-1-carboxylate for tert-butyl (3S)-3-[4-[3-cyano-4-[(2-fluoro-3-pyridyl) sulfanyl] pyrazolo[1,5-a]pyridin-6-yl] pyrazol-1-yl] piperidine-1-carboxylate to give 4-[(5-fluoro-6-methyl-2-pyridyl) sulfanyl]-6-[1-[(3S)-3-piperidyl] pyrazol-4-yl] pyrazolo[1,5-a]pyridine-3-carbonitrile (60 mg, 44% yield) as a white solid. LCMS (MM-ES+APCI, Pos): m/z 434.0 (M+H).

### Example 7

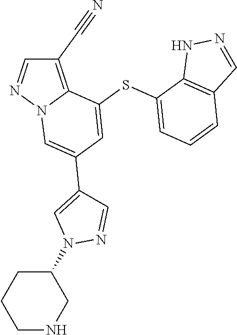 [SMILES: N#Cc1cnn2cc(-c3cnn([C@H]4CCCNC4)c3)cc(Sc3cccc4cn[nH]c34)c12]

### 4-(1H-Indazol-7-ylsulfanyl)-6-[1-[(3S)-3-piperidyl]pyrazol-4-yl]pyrazolo[1,5-a]pyridine-3-carbonitrile (7)

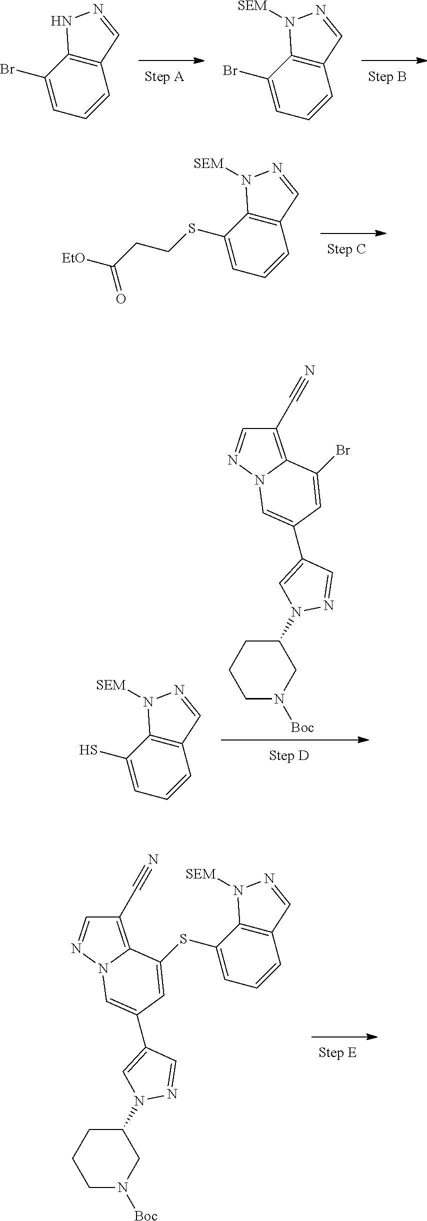 [SMILES: Brc1cccc2cn[nH]c12 ; C[Si](C)(C)CCOCn1ncc2cccc(Br)c21 ; CCOC(=O)CCSc1cccc2cnn(COCC[Si](C)(C)C)c12 ; C[Si](C)(C)CCOCn1ncc2cccc(S)c21 ; CC(C)(C)OC(=O)N1CCC[C@H](n2cc(-c3cc(Br)c4c(C#N)cnn4c3)cn2)C1 ; CC(C)(C)OC(=O)N1CCC[C@H](n2cc(-c3cc(Sc4cccc5cnn(COCC[Si](C)(C)C)c45)c4c(C#N)cnn4c3)cn2)C1]

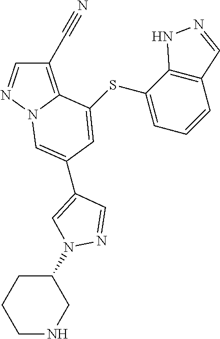 [SMILES: N#Cc1cnn2cc(-c3cnn([C@H]4CCCNC4)c3)cc(Sc3cccc4cn[nH]c34)c12]

Step A. 2-[(7-Bromoindazol-1-yl)methoxy]ethyl-trimethyl-silane. To a solution of 7-bromo-1H-indazole (3.0 g, 15 mmol) in DMF (30 mL) was added SEM-Cl (3.8 g, 23 mmol) and K₂CO₃ (4.2 g, 31 mmol). The mixture was stirred at 50° C. for 2 hr. The mixture was concentrated under reduced pressure, purified by silica gel chromatography (10-20% EA/petroleum ether/ethyl acetate) followed by purification by reversed phase column (58-88% acetonitrile/10 um NH₄HCO₃) to give 2-[(7-bromoindazol-1-yl)methoxy]ethyl-trimethyl-silane (3.9 g, 78% yield) as colorless oil. LCMS (MM-ES+APCI, Pos): m/z 328.8 (M+H).

Step B. Ethyl 3-[1-(2-trimethylsilylethoxymethyl)indazol-7-yl]sulfanylpropanoate. Synthesized according to Example 3, Step A, substituting 2-[(7-bromoindazol-1-yl)methoxy]ethyl-trimethyl-silane for 3-bromo-2-fluoro-pyridine to give ethyl 3-[1-(2-trimethylsilylethoxymethyl)indazol-7-yl]sulfanylpropanoate (0.35 g, 67% yield) as light yellow oil. LCMS (MM-ES+APCI, Pos): m/z 381.8 (M+H).

Step C. 1-(2-Trimethylsilylethoxymethyl)indazole-7-thiol. Synthesized according to Example 3, Step B, substituting ethyl 3-[1-(2-trimethylsilylethoxymethyl)indazol-7-yl]sulfanylpropanoate for ethyl 3-[(2-fluoro-3-pyridyl) sulfanyl] propanoate to give 1-(2-trimethylsilylethoxymethyl)indazole-7-thiol (0.18 g, 73% yield) as light yellow oil. LCMS (MM-ES+APCI, Pos): m/z 281.0 (M+H).

Step D. T-Butyl(3S)-3-[4-[3-cyano-4-[1-(2-trimethylsilylethoxymethyl)indazol-7-yl]sulfanyl-pyrazolo[1,5-a]pyridin-6-yl]pyrazol-1-yl]piperidine-1-carboxylate. Synthesized according to Example 3, Step C, substituting 1-(2-trimethylsilylethoxymethyl)indazole-7-thiol for 2-fluoropyridine-3-thiol to give tert-butyl(3S)-3-[4-[3-cyano-4-[1-(2-trimethylsilylethoxymethyl)indazol-7-yl]sulfanyl-pyrazolo[1,5-a]pyridin-6-yl]pyrazol-1-yl]piperidine-1-carboxylate (0.13 g, 36% yield). LCMS (MM-ES+APCI, Pos): m/z 671.5 (M+H).

Step E. 4-(1H-Indazol-7-ylsulfanyl)-6-[1-[(3S)-3-piperidyl]pyrazol-4-yl]pyrazolo[1,5-a]pyridine-3-carbonitrile. Synthesized according to Example 3, Step D, substituting tert-butyl (3S)-3-[4-[3-cyano-4-[1-(2-trimethylsilylethoxymethyl)indazol-7-yl]sulfanyl-pyrazolo[1,5-a]pyridin-6-yl]pyrazol-1-yl]piperidine-1-carboxylate for tert-butyl (3S)-3-[4-[3-cyano-4-[(2-fluoro-3-pyridyl) sulfanyl] pyrazolo[1,5-a]pyridin-6-yl] pyrazol-1-yl] piperidine-1-carboxylate to give 4-(1H-indazol-7-ylsulfanyl)-6-[1-[(3S)-3-piperidyl]pyrazol-4-yl]pyrazolo[1,5-a]pyridine-3-carbonitrile (24 mg, 29% yield) as yellow solid. LCMS (MM-ES+APCI, Pos): m/z 440.9 (M+H).

### Example 8

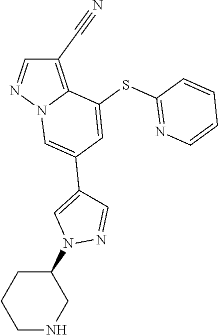 [SMILES: N#Cc1cnn2cc(-c3cnn([C@@H]4CCCNC4)c3)cc(Sc3ccccn3)c12]

### 6-[1-[(3R)-3-Piperidyl]pyrazol-4-yl]-4-(2-pyridylsulfanyl)pyrazolo[1,5-a]pyridine-3-carbonitrile (8)

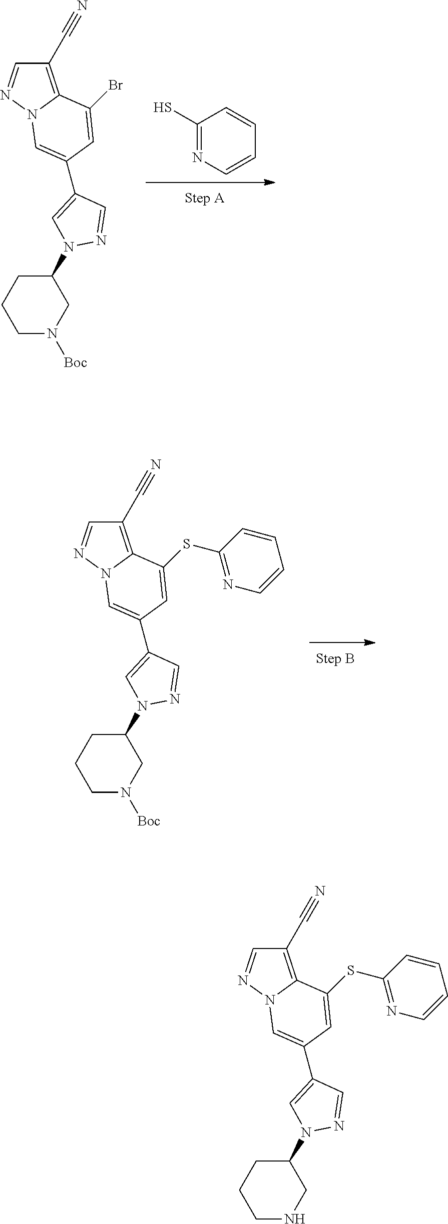 [SMILES: CC(C)(C)OC(=O)N1CCC[C@@H](n2cc(-c3cc(Br)c4c(C#N)cnn4c3)cn2)C1 ; Sc1ccccn1 ; CC(C)(C)OC(=O)N1CCC[C@@H](n2cc(-c3cc(Sc4ccccn4)c4c(C#N)cnn4c3)cn2)C1 ; N#Cc1cnn2cc(-c3cnn([C@@H]4CCCNC4)c3)cc(Sc3ccccn3)c12]

Step A. t-Butyl (3R)-3-[4-[3-cyano-4-(2-pyridylsulfanyl)pyrazolo[1,5-a]pyridin-6-yl]pyrazol-1-yl]piperidine-1-carboxylate. Synthesized according to Example 1, Step A, substituting tert-butyl (3R)-3-[4-(4-bromo-3-cyano-pyrazolo[1,5-a]pyridin-6-yl)pyrazol-1-yl]piperidine-1-carboxylate for tert-butyl (3S)-3-[4-(4-bromo-3-cyano-pyrazolo[1,5-a]pyridin-6-yl)pyrazol-1-yl]piperidine-1-carboxylate to give tert-butyl (3R)-3-[4-[3-cyano-4-(2-pyridylsulfanyl)pyrazolo[1,5-a]pyridin-6-yl]pyrazol-1-yl]piperidine-1-carboxylate (51 mg, 85% yield) as a light yellow solid. LCMS (MM-ES+APCI, Pos): m/z 502.4 (M+H).

Step B. 6-[1-[(3R)-3-Piperidyl]pyrazol-4-yl]-4-(2-pyridylsulfanyl)pyrazolo[1,5-a]pyridine-3-carbonitrile. Synthesized according to Example 1, Step B, substituting (3R)-3-[4-[3-cyano-4-(2-pyridylsulfanyl)pyrazolo[1,5-a]pyridin-6-yl]pyrazol-1-yl]piperidine-1-carboxylate for tert-butyl (3S)-3-[4-[3-cyano-4-(2-pyridylsulfanyl)pyrazolo[1,5-a]pyridin-6-yl]pyrazol-1-yl]piperidine-1-carboxylate to give 6-[1-[(3R)-3-piperidyl]pyrazol-4-yl]-4-(2-pyridylsulfanyl)pyrazolo[1,5-a]pyridine-3-carbonitrile (45 mg, 42% yield) as an off-white solid. LCMS (MM-ES+APCI, Pos): m/z 402.2 (M+H).

### Example 9

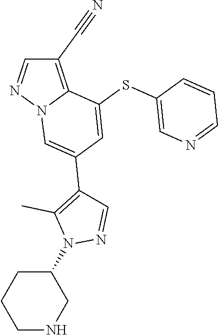 [SMILES: Cc1c(-c2cc(Sc3cccnc3)c3c(C#N)cnn3c2)cnn1[C@H]1CCCNC1]

### 6-[5-Methyl-1-[(3S)-3-piperidyl]pyrazol-4-yl]-4-(3-pyridylsulfanyl)pyrazolo[1,5-a]pyridine-3-carbonitrile (9)

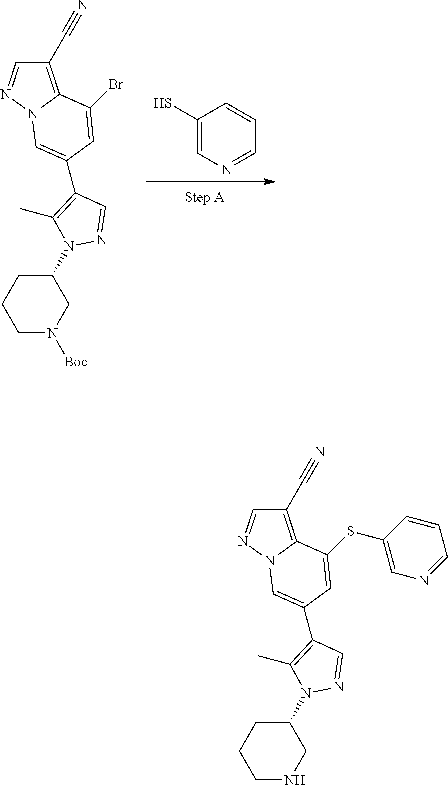 [SMILES: Cc1c(-c2cc(Br)c3c(C#N)cnn3c2)cnn1[C@H]1CCCN(C(=O)OC(C)(C)C)C1 ; Sc1cccnc1 ; Cc1c(-c2cc(Sc3cccnc3)c3c(C#N)cnn3c2)cnn1[C@H]1CCCNC1]

Step A. 6-[5-Methyl-1-[(3S)-3-piperidyl]pyrazol-4-yl]-4-(3-pyridylsulfanyl)pyrazolo[1,5-a]pyridine-3-carbonitrile. To a 8 mL vial equipped with a magnetic stir bar was added tert-butyl (3S)-3-[4-(4-bromo-3-cyano-pyrazolo[1,5-a]pyridin-6-yl)-5-methyl-pyrazol-1-yl]piperidine-1-carboxylate (50 mg, 0.089 mmol) and pyridine-3-thiol (13 mg, 0.11 mmol) followed by the addition of DMF (2 mL). Cs₂CO₃ (58 mg, 0.18 mmol), CuI (5.1 mg, 0.027 mmol) and 1,10-phenanthroline (4.8 mg, 0.027 mmol) were added to the mixture at 25° C. The mixture was stirred at 120° C. for 48 h. The suspension was filtered, and filter cake washed with MeOH (5 mL). The concentrate was purified by preparative HPLC: (25%-55% acetonitrile/0.04% aqueous ammonia hydroxide+10 mM NH₄HCO₃). After lyophilization, 6-[5-methyl-1-[(3S)-3-piperidyl]pyrazol-4-yl]-4-(3-pyridylsulfanyl)pyrazolo[1,5-a]pyridine-3-carbonitrile (11 mg, 30% yield,) was obtained as an off-white solid. LCMS (MM-ES+APCI, Pos): m/z 416.1 (M+H).

### Example 10

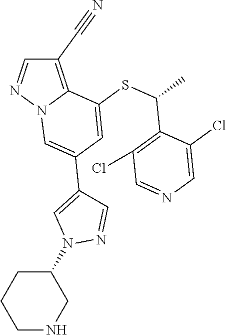 [SMILES: C[C@@H](Sc1cc(-c2cnn([C@H]3CCCNC3)c2)cn2ncc(C#N)c12)c1c(Cl)cncc1Cl]

### 4-[(1R)-1-(3,5-Dichloro-4-pyridyl)ethyl]sulfanyl-6-[1-[(3S)-3-piperidyl]pyrazol-4-yl]pyrazolo[1,5-a]pyridine-3-carbonitrile (10)

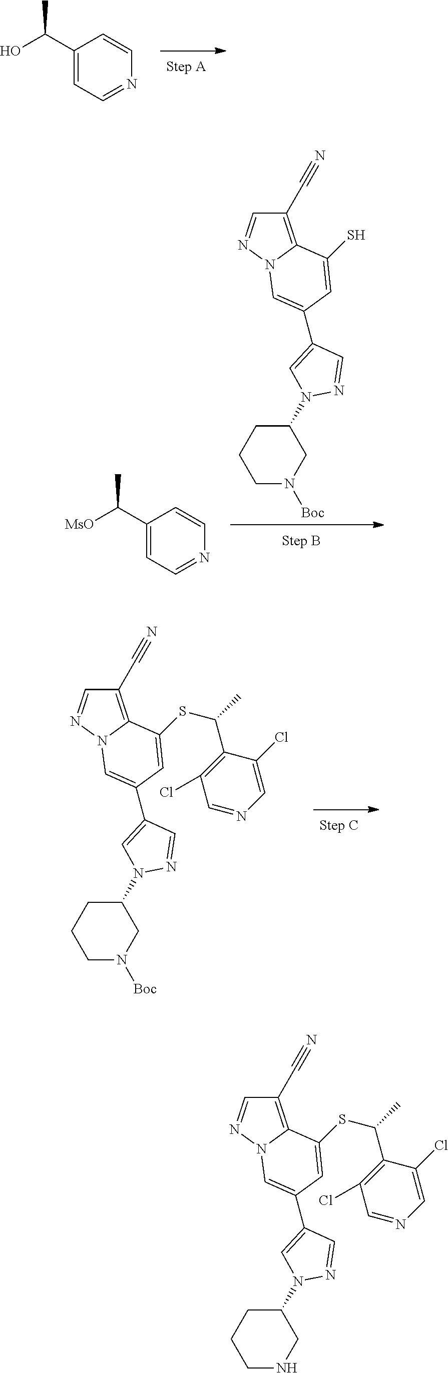

Step A. [(1S)-1-(3,5-Dichloro-4-pyridyl)ethyl] methanesulfonate. To a 40 mL vial equipped with a magnetic stir bar were added (1S)-1-(3,5-dichloro-4-pyridyl)ethanol (1.0 g, 5.2 mmol) and triethylamine (1.0 mL, 7.8 mmol) followed by the addition of DCM (9 mL). The solution was cooled to 0° C. Methylsulfonyl methanesulfonate (2.3 g, 13 mmol) in DCM (3 mL) was added dropwise. The mixture was warmed to 25° C. and stirred for 15 minutes. DMAP (63 mg, 0.52 mmol) in DCM (0.5 mL) was added dropwise. The mixture was stirred at 25° C. for 1 h. Water (30 mL) was added to the reaction and the mixture was transferred to a separatory funnel. The aqueous mixture was extracted with ethyl acetate (30 mL×3). The combined organic layers were washed with brine (30 mL×3), dried over anhydrous sodium sulfate, filtered, and concentrated under reduced pressure to give [(1S)-1-(3,5-dichloro-4-pyridyl)ethyl] methanesulfonate (1.3 g, 92% yield), was used in the next step without further purification.

Step B. t-Butyl (3S)-3-[4-(3-cyano-4-sulfanyl-pyrazolo[1,5-a]pyridin-6-yl)pyrazol-1-yl]piperidine-1-carboxylate. To a 40 mL vial equipped with a magnetic stir bar was added tert-butyl (3S)-3-[4-(3-cyano-4-sulfanyl-pyrazolo[1,5-a]pyridin-6-yl)pyrazol-1-yl]piperidine-1-carboxylate (0.15 g, 0.35 mmol) and [(1S)-1-(3,5-dichloro-4-pyridyl)ethyl] methanesulfonate (0.12 g, 0.42 mmol) followed by the addition of DMF (3 mL). Cs₂CO₃ (0.17 mg, 0.53 mmol) was added to the mixture at 25° C. and stirred at 90° C. for 1 h. Water (10 mL) was added to the reaction and the mixture was transferred to a separatory funnel. The aqueous layer was extracted with ethyl acetate (10 mL×3). The combined organic layers were washed with brine (35 mL), dried over anhydrous sodium sulfate, filtered, concentrated under reduced pressure and purified by silica gel chromatography (50-60% EtOAc/petroleum ether) to give tert-butyl (3S)-3-[4-[3-cyano-4-[(1R)-1-(3,5-dichloro-4-pyridyl)ethyl]sulfanyl-pyrazolo[1,5-a]pyridin-6-yl]pyrazol-1-yl] piperidine-1-carboxylate (0.18 g, 84% yield) as a yellow solid. LCMS (MM-ES+APCI, Pos): m/z 598.2 (M+H).

Step C. 4-[(1R)-1-(3,5-Dichloro-4-pyridyl)ethyl]sulfanyl-6-[1-[(3S)-3-piperidyl]pyrazol-4-yl]pyrazolo[1,5-a]pyridine-3-carbonitrile. Synthesized according to Example 1, Step B, substituting tert-butyl (3S)-3-[4-[3-cyano-4-[(1R)-1-(3,5-dichloro-4-pyridyl)ethyl]sulfanyl-pyrazolo[1,5-a]pyridin-6-yl]pyrazol-1-yl]piperidine-1-carboxylate for tert-butyl (3S)-3-[4-[3-cyano-4-(2-pyridylsulfanyl)pyrazolo[1,5-a]pyridin-6-yl]pyrazol-1-yl]piperidine-1-carboxylate to give 4-[(1R)-1-(3,5-dichloro-4-pyridyl)ethyl]sulfanyl-6-[1-[(3S)-3-piperidyl]pyrazol-4-yl]pyrazolo[1,5-a]pyridine-3-carbonitrile (70 mg, 53% yield) as a white solid. LCMS (MM-ES+APCI, Pos): m/z 499.9 (M+H).

### Example 11

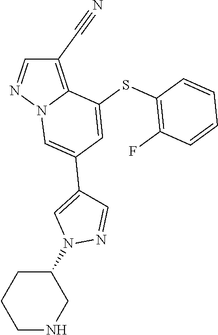 [SMILES: N#Cc1cnn2cc(-c3cnn([C@H]4CCCNC4)c3)cc(Sc3ccccc3F)c12]

### 4-(2-Fluorophenyl)sulfanyl-6-[1-[(3S)-3-piperidyl]pyrazol-4-yl]pyrazolo[1,5-a]pyridine-3-carbonitrile (11)

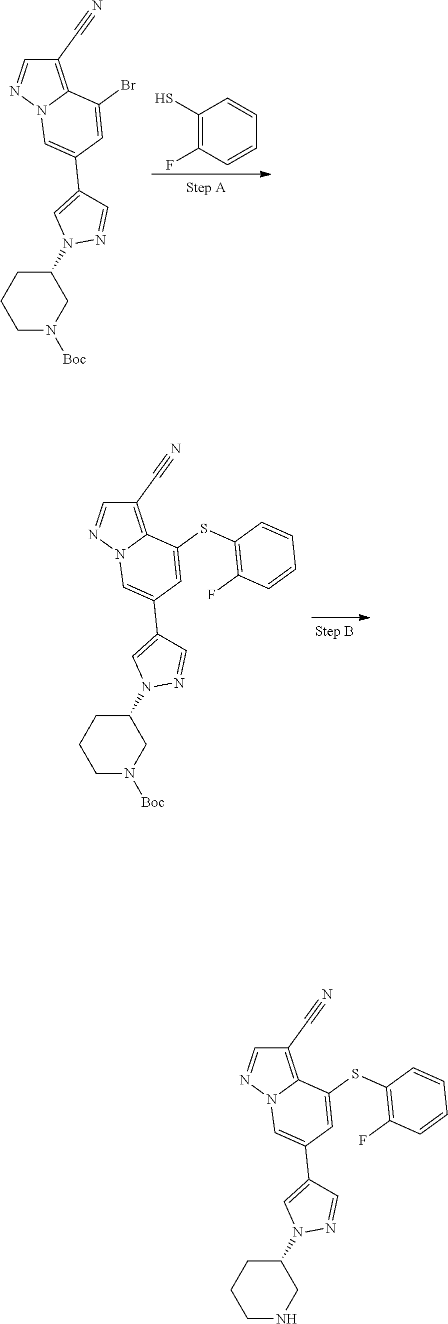

Step A. t-Butyl (3R)-3-[4-[3-cyano-4-(2-pyridylsulfanyl)pyrazolo[1,5-a]pyridin-6-yl]pyrazol-1-yl]piperidine-1-carboxylate. Synthesized according to Example 3, Step C, substituting 2-fluorothiophenol for 2-fluoropyridine-3-thiol to give tert-butyl (3S)-3-[4-[3-cyano-4-(2-fluorophenyl)sulfanyl-pyrazolo[1,5-a]pyridin-6-yl]pyrazol-1-yl]piperidine-1-carboxylate (0.18 g, 99% yield) as a yellow oil. LCMS (MM-ES+APCI, Pos): m/z 519.2 (M+H).

Step B. 6-[1-[(3R)-3-Piperidyl]pyrazol-4-yl]-4-(2-pyridylsulfanyl)pyrazolo[1,5-a]pyridine-3-carbonitrile. To a 20 mL round-bottom flask equipped with a magnetic stir bar was added tert-butyl (3S)-3-[4-[3-cyano-4-(2-fluorophenyl)sulfanyl-pyrazolo[1,5-a]pyridin-6-yl]pyrazol-1-yl]piperidine-1-carboxylate (0.17 g, 0.31 mmol) followed by the addition of DCM (2 mL). TFA (0.40 mL, 5.4 mmol) was added to the mixture and stirred at 25° C. for 0.5 h. The mixture was concentrated under reduced pressure and purified by preparative HPLC: (31%-61% acetonitrile/0.05% aqueous ammonia hydroxide+10 mM NH₄HCO₃). After lyophilization, 4-(2-fluorophenyl)sulfanyl-6-[1-[(3S)-3-piperidyl]pyrazol-4-yl]pyrazolo[1,5-a]pyridine-3-carbonitrile (0.10 g, 72% yield) was obtained as a white solid. LCMS (MM-ES+APCI, Pos): m/z 419.1 (M+H).

### Example 12

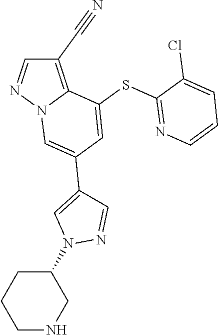 [SMILES: N#Cc1cnn2cc(-c3cnn([C@H]4CCCNC4)c3)cc(Sc3ncccc3Cl)c12]

### 4-[(3-Chloro-2-pyridyl) sulfanyl]-6-[1-[(3S)-3-piperidyl] pyrazol-4-yl] pyrazolo[1,5-a] pyridine-3-carbonitrile (12)

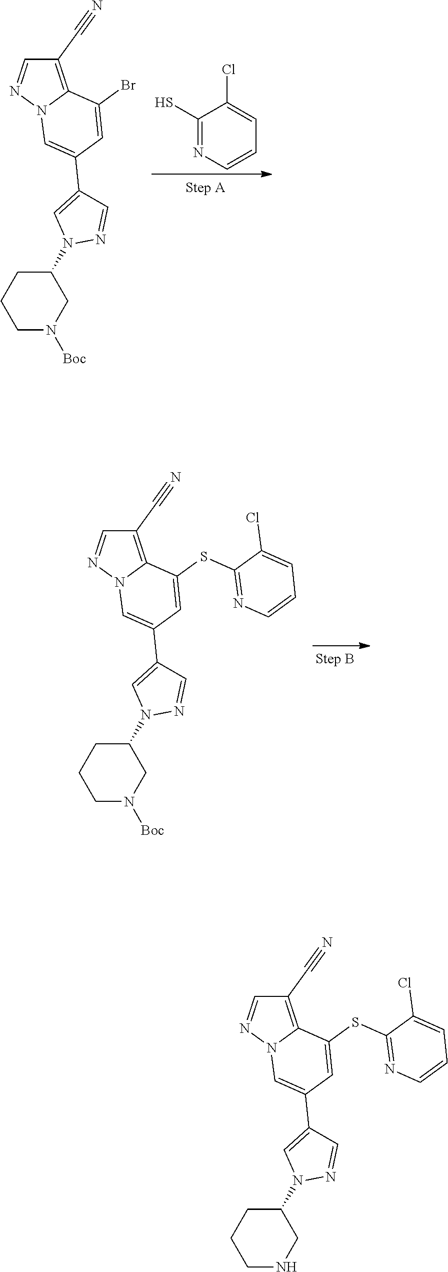 [SMILES: CC(C)(C)OC(=O)N1CCC[C@H](n2cc(-c3cc(Br)c4c(C#N)cnn4c3)cn2)C1 ; Sc1ncccc1Cl ; CC(C)(C)OC(=O)N1CCC[C@H](n2cc(-c3cc(Sc4ncccc4Cl)c4c(C#N)cnn4c3)cn2)C1 ; N#Cc1cnn2cc(-c3cnn([C@H]4CCCNC4)c3)cc(Sc3ncccc3Cl)c12]

Step A. t-Butyl (3S)-3-[4-[4-[(3-chloro-2-pyridyl)sulfanyl]-3-cyano-pyrazolo[1,5-a]pyridin-6-yl]pyrazol-1-yl]piperidine-1-carboxylate. Synthesized according to Example 3, Step C, substituting 3-chloropyridine-2-thiol for 2-fluoropyridine-3-thiol to give tert-butyl (3S)-3-[4-[4-[(3-chloro-2-pyridyl)sulfanyl]-3-cyano-pyrazolo[1,5-a]pyridin-6-yl]pyrazol-1-yl]piperidine-1-carboxylate (0.17 g, crude) as a yellow solid. LCMS (MM-ES+APCI, Pos): m/z 558.4 (M+Na⁺).

Step B. 4-[(3-Chloro-2-pyridyl) sulfanyl]-6-[1-[(3S)-3-piperidyl] pyrazol-4-yl] pyrazolo[1, 5-a]pyridine-3-carbonitrile. Synthesized according to Example 11, Step B, substituting tert-butyl (3S)-3-[4-[4-[(3-chloro-2-pyridyl) sulfanyl]-3-cyano-pyrazolo[1,5-a]pyridin-6-yl] pyrazol-1-yl] piperidine-1-carboxylate for tert-butyl (3S)-3-[4-[3-cyano-4-(2-fluorophenyl)sulfanyl-pyrazolo[1,5-a]pyridin-6-yl]pyrazol-1-yl]piperidine-1-carboxylate to give 4-[(3-chloro-2-pyridyl) sulfanyl]-6-[1-[(3S)-3-piperidyl] pyrazol-4-yl] pyrazolo[1,5-a] pyridine-3-carbonitrile (41 mg, 34% yield) as an off-white solid. LCMS (MM-ES+APCI, Pos): m/z 436.0 (M+H).

### Example 13

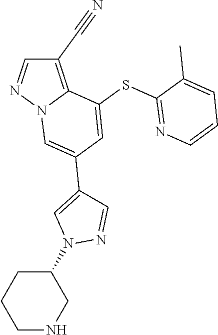 [SMILES: Cc1cccnc1Sc1cc(-c2cnn([C@H]3CCCNC3)c2)cn2ncc(C#N)c12]

### 4-[(3-Methyl-2-pyridyl) sulfanyl]-6-[1-[(3S)-3-piperidyl] pyrazol-4-yl] pyrazolo[1,5-a] pyridine-3-carbonitrile (13)

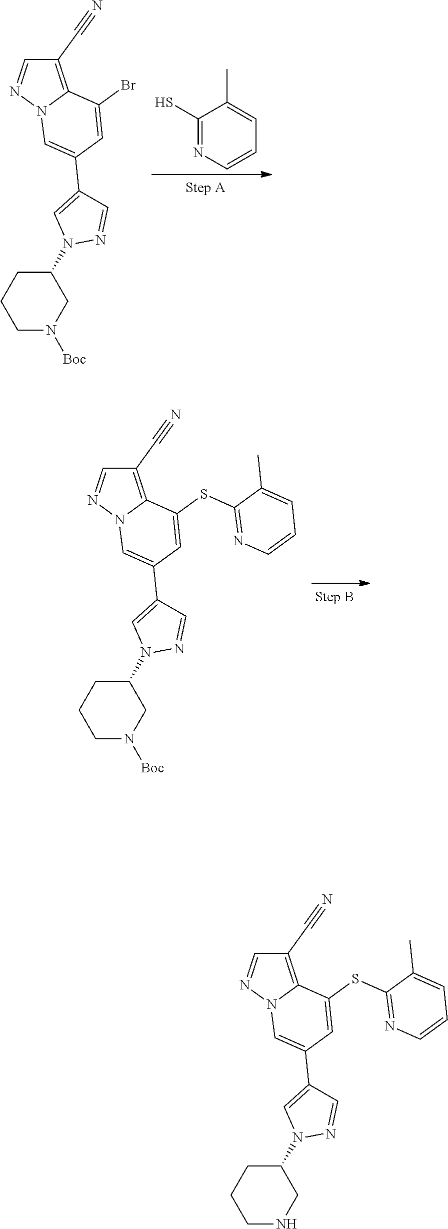

Step A. t-Butyl (3S)-3-[4-[3-cyano-4-[(3-methyl-2-pyridyl) sulfanyl] pyrazolo[1,5-a] pyridin-6-yl] pyrazol-1-yl] piperidine-1-carboxylate. Synthesized according to Example 3, Step C, substituting 3-methylpyridine-2-thiol for 2-fluoropyridine-3-thiol to give tert-butyl (3S)-3-[4-[3-cyano-4-[(3-methyl-2-pyridyl) sulfanyl] pyrazolo[1,5-a]pyridin-6-yl] pyrazol-1-yl] piperidine-1-carboxylate (0.26 g, crude) as a yellow solid.

Step B. 4-[(3-Methyl-2-pyridyl) sulfanyl]-6-[1-[(3S)-3-piperidyl] pyrazol-4-yl] pyrazolo [1,5-a]pyridine-3-carbonitrile. Synthesized according to Example 11, Step B, substituting tert-butyl (3S)-3-[4-[3-cyano-4-[(3-methyl-2-pyridyl)sulfanyl]pyrazolo[1,5-a]pyridin-6-yl]pyrazol-1-yl]piperidine-1-carboxylate for tert-butyl (3S)-3-[4-[3-cyano-4-(2-fluorophenyl)sulfanyl-pyrazolo[1,5-a]pyridin-6-yl]pyrazol-1-yl]piperidine-1-carboxylate to give 4-[(3-methyl-2-pyridyl) sulfanyl]-6-[1-[(3S)-3-piperidyl] pyrazol-4-yl] pyrazolo[1,5-a] pyridine-3-carbonitrile (50 mg, 27% yield) as a white solid. LCMS (MM-ES+APCI, Pos): m/z 416.2 (M+H).

### Example 14

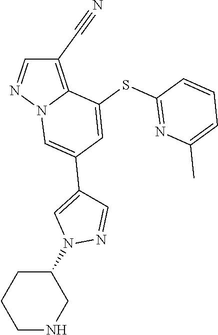 [SMILES: Cc1cccc(Sc2cc(-c3cnn([C@H]4CCCNC4)c3)cn3ncc(C#N)c23)n1]

### 4-[(6-Methyl-2-pyridyl)sulfanyl]-6-[1-[(3S)-3-piperidyl]pyrazol-4-yl]pyrazolo[1,5-a]pyridine-3-carbonitrile (14)

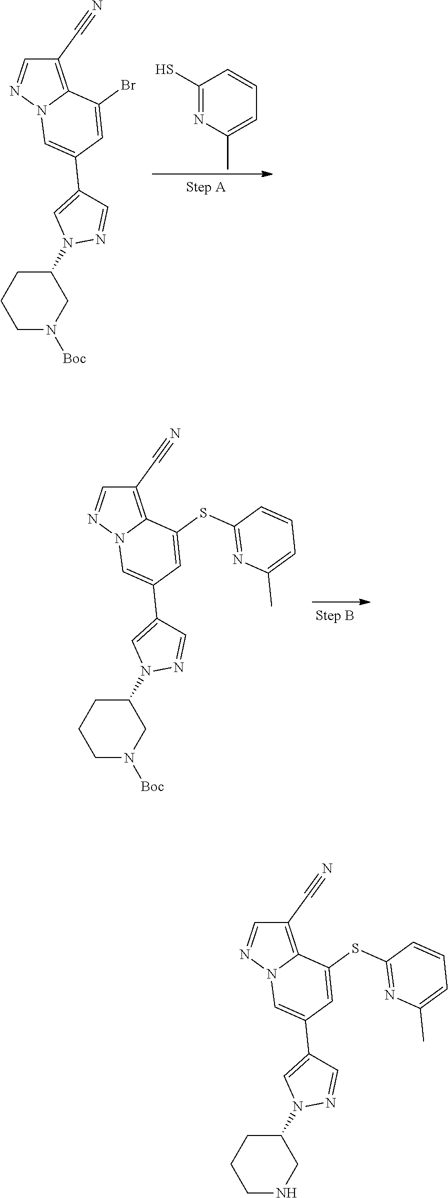

Step A. t-Butyl (3S)-3-[4-[3-cyano-4-[(3-methyl-2-pyridyl) sulfanyl] pyrazolo[1,5-a] pyridin-6-yl] pyrazol-1-yl] piperidine-1-carboxylate. Synthesized according to Example 3, Step C, substituting 6-methylpyridine-2-thiol for 2-fluoropyridine-3-thiol to give tert-butyl (3S)-3-[4-[3-cyano-4-[(6-methyl-2-pyridyl)sulfanyl] pyrazolo[1,5-a]pyridin-6-yl]pyrazol-1-yl]piperidine-1-carboxylate (0.15 g, 74% yield). LCMS (MM-ES+APCI, Pos): m/z 516.5 (M+H).

Step B. 4-[(6-Methyl-2-pyridyl)sulfanyl]-6-[1-[(3S)-3-piperidyl]pyrazol-4-yl]pyrazolo[1,5-a]pyridine-3-carbonitrile. Synthesized according to Example 11, Step B, substituting tert-butyl (3S)-3-[4-[3-cyano-4-[(6-methyl-2-pyridyl)sulfanyl]pyrazolo[1,5-a]pyridin-6-yl]pyrazol-1-yl]piperidine-1-carboxylate for tert-butyl (3S)-3-[4-[3-cyano-4-(2-fluorophenyl)sulfanyl-pyrazolo[1,5-a]pyridin-6-yl]pyrazol-1-yl]piperidine-1-carboxylate to give 4-[(6-methyl-2-pyridyl)sulfanyl]-6-[1-[(3S)-3-piperidyl]pyrazol-4-yl]pyrazolo[1,5-a]pyridine-3-carbonitrile (48 mg, 35% yield) as a white solid. LCMS (MM-ES+APCI, Pos): m/z 416.2 (M+H).

### Example 15

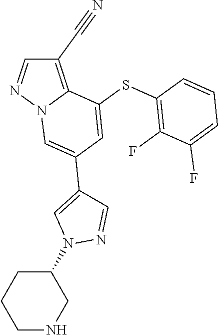 [SMILES: N#Cc1cnn2cc(-c3cnn([C@H]4CCCNC4)c3)cc(Sc3cccc(F)c3F)c12]

### 4-(2,3-Difluorophenyl)sulfanyl-6-[1-[(3S)-3-piperidyl]pyrazol-4-yl]pyrazolo[1,5-a]pyridine-3-carbonitrile (15)

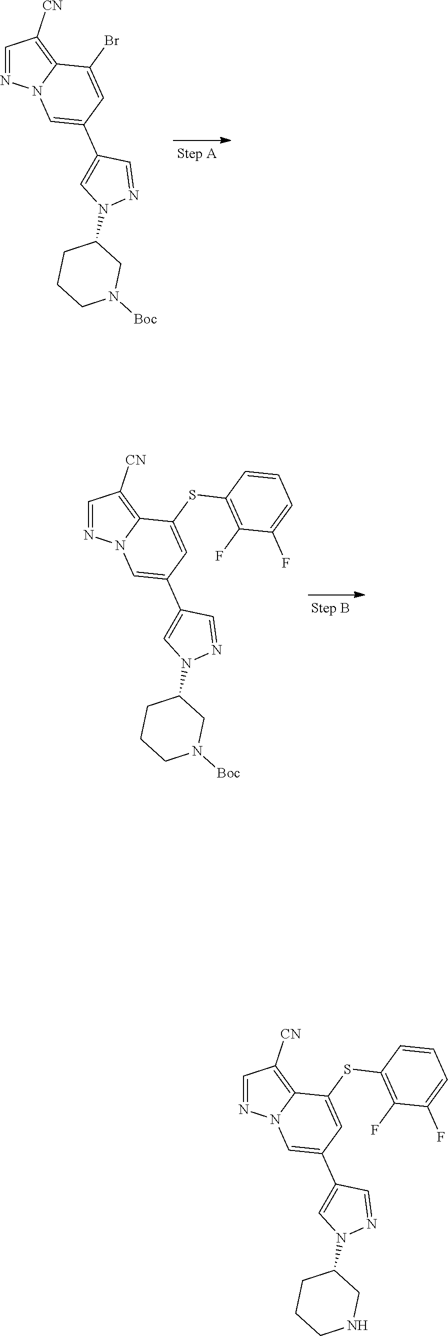 [SMILES: CC(C)(C)OC(=O)N1CCC[C@H](n2cc(-c3cc(Br)c4c(C#N)cnn4c3)cn2)C1 ; CC(C)(C)OC(=O)N1CCC[C@H](n2cc(-c3cc(Sc4cccc(F)c4F)c4c(C#N)cnn4c3)cn2)C1 ; N#Cc1cnn2cc(-c3cnn([C@H]4CCCNC4)c3)cc(Sc3cccc(F)c3F)c12]

Step A. t-Butyl (3S)-3-[4-[3-cyano-4-(2,3-difluorophenyl)sulfanyl-pyrazolo[1,5-a]pyridin-6-yl]pyrazol-1-yl]piperidine-1-carboxylate. Synthesized according to Example 3, Step C, substituting 2,3-difluorobenzenethiol for 2-fluoropyridine-3-thiol to give tert-butyl (3S)-3-[4-[3-cyano-4-(2,3-difluorophenyl)sulfanyl-pyrazolo[1,5-a]pyridin-6-yl]pyrazol-1-yl]piperidine-1-carboxylate (0.16 g, 74% yield) as a yellow oil. LCMS (MM-ES+APCI, Pos): m/z 537.3 (M+H).

Step B. 4-(2,3-Difluorophenyl)sulfanyl-6-[1-[(3S)-3-piperidyl]pyrazol-4-yl]pyrazolo[1,5-a]pyridine-3-carbonitrile. Synthesized according to Example 1, Step B, substituting tert-butyl (3S)-3-[4-[3-cyano-4-(2,3-difluorophenyl)sulfanyl-pyrazolo[1,5-a]pyridin-6-yl]pyrazol-1-yl]piperidine-1-carboxylate for tert-butyl (3S)-3-[4-[3-cyano-4-(2-pyridylsulfanyl)pyrazolo[1,5-a]pyridin-6-yl]pyrazol-1-yl]piperidine-1-carboxylate to give 4-(2,3-difluorophenyl)sulfanyl-6-[1-[(3S)-3-piperidyl]pyrazol-4-yl]pyrazolo[1,5-a]pyridine-3-carbonitrile (16 mg, 17% yield) as a white solid. LCMS (MM-ES+APCI, Pos): m/z 437.1 (M+H).

### Example 16

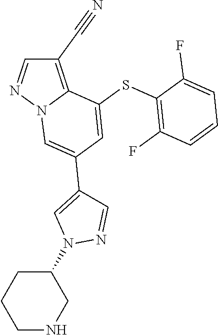 [SMILES: N#Cc1cnn2cc(-c3cnn([C@H]4CCCNC4)c3)cc(Sc3c(F)cccc3F)c12]

### 4-(2,6-Difluorophenyl)sulfanyl-6-[1-[(3S)-3-piperidyl]pyrazol-4-yl]pyrazolo[1,5-a]pyridine-3-carbonitrile (16)

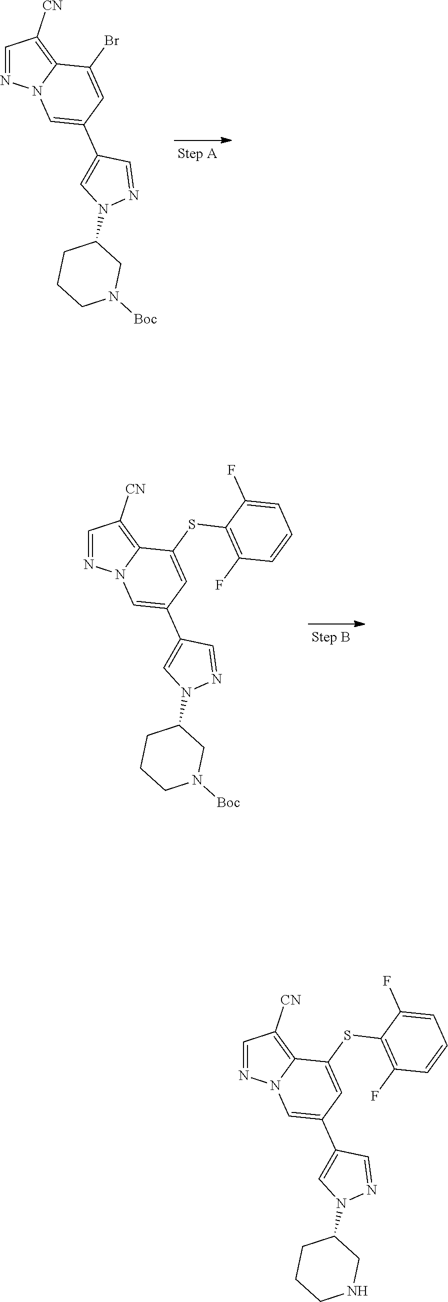 [SMILES: CC(C)(C)OC(=O)N1CCC[C@H](n2cc(-c3cc(Br)c4c(C#N)cnn4c3)cn2)C1 ; CC(C)(C)OC(=O)N1CCC[C@H](n2cc(-c3cc(Sc4c(F)cccc4F)c4c(C#N)cnn4c3)cn2)C1 ; N#Cc1cnn2cc(-c3cnn([C@H]4CCCNC4)c3)cc(Sc3c(F)cccc3F)c12]

Step A. t-Butyl (3S)-3-[4-[3-cyano-4-(2,3-difluorophenyl)sulfanyl-pyrazolo[1,5-a]pyridin-6-yl]pyrazol-1-yl]piperidine-1-carboxylate. Synthesized according to Example 3, Step C, substituting 2,6-difluorobenzenethiol for 2-fluoropyridine-3-thiol to give tert-butyl (3S)-3-[4-[3-cyano-4-(2,6-difluorophenyl)sulfanyl-pyrazolo[1,5-a]pyridin-6-yl]pyrazol-1-yl]piperidine-1-carboxylate (0.22 g, 54% yield) as a yellow oil. LCMS (MM-ES+APCI, Pos): m/z 537.3 (M+H).

Step B. 4-(2,6-Difluorophenyl)sulfanyl-6-[1-[(3S)-3-piperidyl]pyrazol-4-yl]pyrazolo[1,5-a]pyridine-3-carbonitrile. Synthesized according to Example 11, Step B, substituting tert-butyl (3S)-3-[4-[3-cyano-4-(2,6-difluorophenyl)sulfanyl-pyrazolo[1,5-a]pyridin-6-yl] pyrazol-1-yl]piperidine-1-carboxylate for tert-butyl (3S)-3-[4-[3-cyano-4-(2-fluorophenyl)sulfanyl-pyrazolo[1,5-a]pyridin-6-yl]pyrazol-1-yl]piperidine-1-carboxylate to give 4-(2,6-difluorophenyl)sulfanyl-6-[1-[(3S)-3-piperidyl]pyrazol-4-yl]pyrazolo[1,5-a]pyridine-3-carbonitrile (80 mg, 54% yield) as a white solid. LCMS (MM-ES+APCI, Pos): m/z 437.1 (M+H).

### Example 17

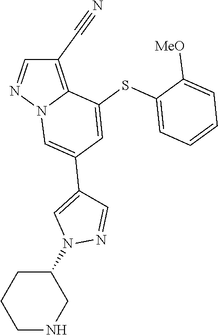 [SMILES: COc1ccccc1Sc1cc(-c2cnn([C@H]3CCCNC3)c2)cn2ncc(C#N)c12]

### 4-(2-Methoxyphenyl)sulfanyl-6-[1-[(3S)-3-piperidyl]pyrazol-4-yl]pyrazolo[1,5-a]pyridine-3-carbonitrile (17)

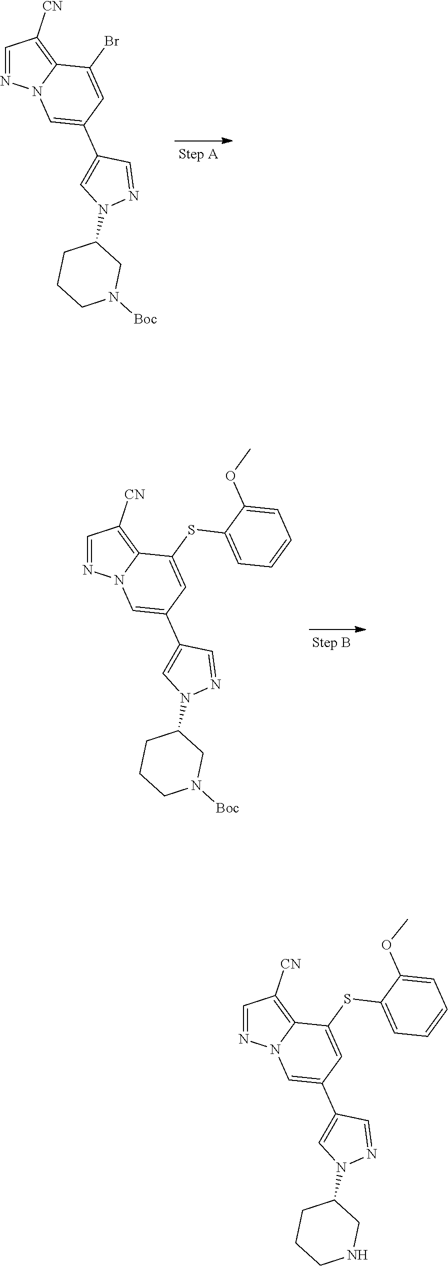 [SMILES: CC(C)(C)OC(=O)N1CCC[C@H](n2cc(-c3cc(Br)c4c(C#N)cnn4c3)cn2)C1 ; COc1ccccc1Sc1cc(-c2cnn([C@H]3CCCN(C(=O)OC(C)(C)C)C3)c2)cn2ncc(C#N)c12 ; COc1ccccc1Sc1cc(-c2cnn([C@H]3CCCNC3)c2)cn2ncc(C#N)c12]

Step A. t-Butyl (3S)-3-[4-[3-cyano-4-(2-methoxyphenyl) sulfanyl-pyrazolo[1,5-a]pyridin-6-yl]pyrazol-1-yl]piperidine-1-carboxylate. Synthesized according to Example 3, Step C, substituting 2-methoxybenzenethiol for 2-fluoropyridine-3-thiol to give tert-butyl (3S)-3-[4-[3-cyano-4-(2-methoxyphenyl) sulfanyl-pyrazolo[1,5-a]pyridin-6-yl]pyrazol-1-yl]piperidine-1-carboxylate (0.14 g, 63% yield) as a yellow oil. LCMS (MM-ES+APCI, Pos): m/z 531.3 (M+H).

Step B. 4-(2-Methoxyphenyl)sulfanyl-6-[1-[(3S)-3-piperidyl]pyrazol-4-yl]pyrazolo[1,5-a]pyridine-3-carbonitrile. Synthesized according to Example 1, Step B, substituting tert-butyl (3S)-3-[4-[3-cyano-4-(2-methoxyphenyl)sulfanyl-pyrazolo[1,5-a]pyridin-6-yl]pyrazol-1-yl]piperidine-1-carboxylate for tert-butyl (3S)-3-[4-[3-cyano-4-(2-pyridylsulfanyl)pyrazolo[1,5-a]pyridin-6-yl]pyrazol-1-yl]piperidine-1-carboxylate to give 4-(2-methoxyphenyl)sulfanyl-6-[1-[(3S)-3-piperidyl]pyrazol-4-yl]pyrazolo[1,5-a]pyridine-3-carbonitrile (55 mg, 64% yield) as pink solid. LCMS (MM-ES+APCI, Pos): m/z 431.1 (M+H).

### Example 18

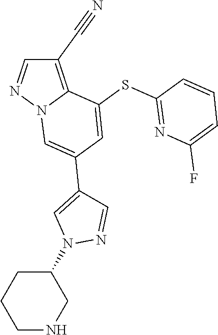 [SMILES: N#Cc1cnn2cc(-c3cnn([C@H]4CCCNC4)c3)cc(Sc3cccc(F)n3)c12]

### 4-[(6-Fluoro-2-pyridyl)sulfanyl]-6-[1-[(3S)-3-piperidyl]pyrazol-4-yl]pyrazolo[1,5-a]pyridine-3-carbonitrile (18)

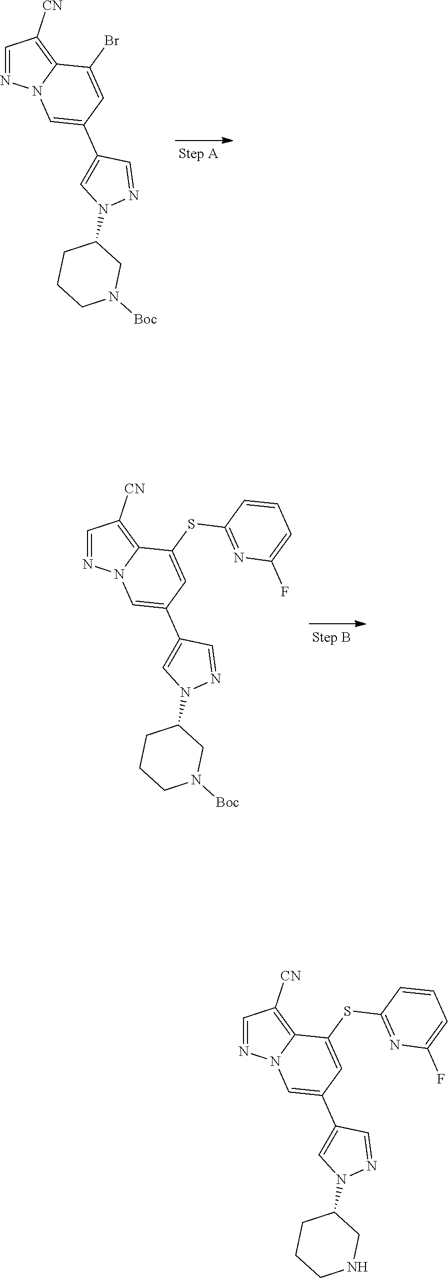 [SMILES: CC(C)(C)OC(=O)N1CCC[C@H](n2cc(-c3cc(Br)c4c(C#N)cnn4c3)cn2)C1 ; CC(C)(C)OC(=O)N1CCC[C@H](n2cc(-c3cc(Sc4cccc(F)n4)c4c(C#N)cnn4c3)cn2)C1 ; N#Cc1cnn2cc(-c3cnn([C@H]4CCCNC4)c3)cc(Sc3cccc(F)n3)c12]

Step A. t-Butyl (3S)-3-[4-[3-cyano-4-[(6-fluoro-2-pyridyl)sulfanyl] pyrazolo[1,5-a]pyridin-6-yl]pyrazol-1-yl]piperidine-1-carboxylate. Synthesized according to Example 3, Step C, substituting 6-fluoropyridine-2-thiol for 2-fluoropyridine-3-thiol to give tert-butyl (3S)-3-[4-[3-cyano-4-[(6-fluoro-2-pyridyl)sulfanyl] pyrazolo[1,5-a]pyridin-6-yl]pyrazol-1-yl]piperidine-1-carboxylate (0.14 g, 67% yield) as a yellow solid. LCMS (MM-ES+APCI, Pos): m/z 464.3 (M-tBu+H).

Step B. 4-[(6-Fluoro-2-pyridyl)sulfanyl]-6-[1-[(3S)-3-piperidyl]pyrazol-4-yl]pyrazolo[1,5-a]pyridine-3-carbonitrile. Synthesized according to Example 1, Step B, substituting tert-butyl (3S)-3-[4-[3-cyano-4-[(6-fluoro-2-pyridyl)sulfanyl]pyrazolo[1,5-a]pyridin-6-yl]pyrazol-1-yl]piperidine-1-carboxylate for tert-butyl (3S)-3-[4-[3-cyano-4-(2-pyridylsulfanyl)pyrazolo[1,5-a]pyridin-6-yl]pyrazol-1-yl]piperidine-1-carboxylate to give 4-[(6-fluoro-2-pyridyl)sulfanyl]-6-[1-[(3S)-3-piperidyl]pyrazol-4-yl]pyrazolo[1,5-a]pyridine-3-carbonitrile (37 mg, 44% yield) as a white solid. LCMS (MM-ES+APCI, Pos): m/z 420.3 (M+H).

### Example 19

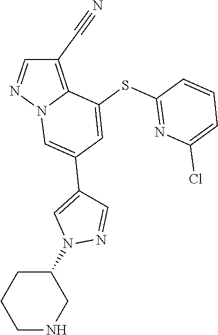 [SMILES: N#Cc1cnn2cc(-c3cnn([C@H]4CCCNC4)c3)cc(Sc3cccc(Cl)n3)c12]

### 4-[(6-Chloro-2-pyridyl) sulfanyl]-6-[1-[(3S)-3-piperidyl]pyrazol-4-yl]pyrazolo[1,5-a]pyridine-3-carbonitrile (19)

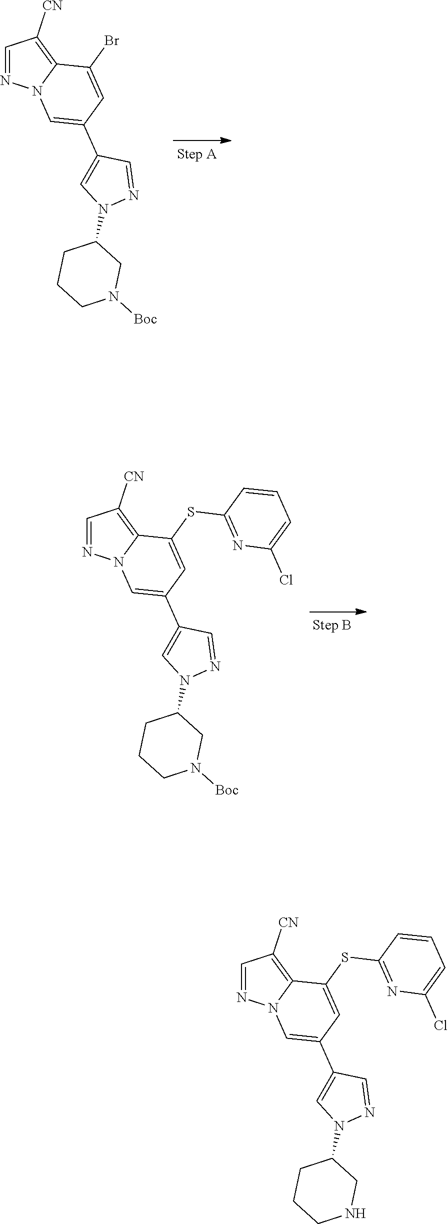 [SMILES: CC(C)(C)OC(=O)N1CCC[C@H](n2cc(-c3cc(Br)c4c(C#N)cnn4c3)cn2)C1 ; CC(C)(C)OC(=O)N1CCC[C@H](n2cc(-c3cc(Sc4cccc(Cl)n4)c4c(C#N)cnn4c3)cn2)C1 ; N#Cc1cnn2cc(-c3cnn([C@H]4CCCNC4)c3)cc(Sc3cccc(Cl)n3)c12]

Step A. t-Butyl (3S)-3-[4-[4-[(6-chloro-2-pyridyl)sulfanyl]-3-cyano-pyrazolo[1,5-a]pyridin-6-yl]pyrazol-1-yl]piperidine-1-carboxylate. Synthesized according to Example 3, Step C, substituting 6-chloropyridine-2-thiol for 2-fluoropyridine-3-thiol to give tert-butyl (3S)-3-[4-[4-[(6-chloro-2-pyridyl)sulfanyl]-3-cyano-pyrazolo[1,5-a]pyridin-6-yl]pyrazol-1-yl]piperidine-1-carboxylate (0.15 g, 59% yield) as a light yellow oil. LCMS (MM-ES+APCI, Pos): m/z 536.3 (M+H).

Step B. 4-[(6-Chloro-2-pyridyl) sulfanyl]-6-[1-[(3S)-3-piperidyl]pyrazol-4-yl]pyrazolo[1,5-a]pyridine-3-carbonitrile. Synthesized according to Example 1, Step B, substituting tert-butyl (3S)-3-[4-[4-[(6-chloro-2-pyridyl)sulfanyl]-3-cyano-pyrazolo[1,5-a]pyridin-6-yl]pyrazol-1-yl]piperidine-1-carboxylate for tert-butyl (3S)-3-[4-[3-cyano-4-(2-pyridylsulfanyl)pyrazolo[1,5-a]pyridin-6-yl]pyrazol-1-yl]piperidine-1-carboxylate to give 4-[(6-chloro-2-pyridyl) sulfanyl]-6-[1-[(3S)-3-piperidyl]pyrazol-4-yl]pyrazolo[1,5-a]pyridine-3-carbonitrile (56 mg, 55% yield) as a white solid. LCMS (MM-ES+APCI, Pos): m/z 436.1 (M+H).

### Example 20

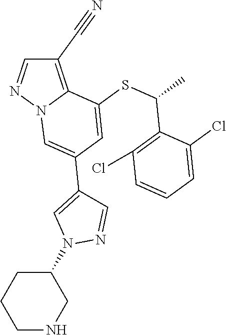 [SMILES: C[C@@H](Sc1cc(-c2cnn([C@H]3CCCNC3)c2)cn2ncc(C#N)c12)c1c(Cl)cccc1Cl]

### 4-[(1R)-1-(2,6-Dichlorophenyl)ethyl]sulfanyl-6-[1-[(3S)-3-piperidyl]pyrazol-4-yl]pyrazolo[1,5-a]pyridine-3-carbonitrile (20)

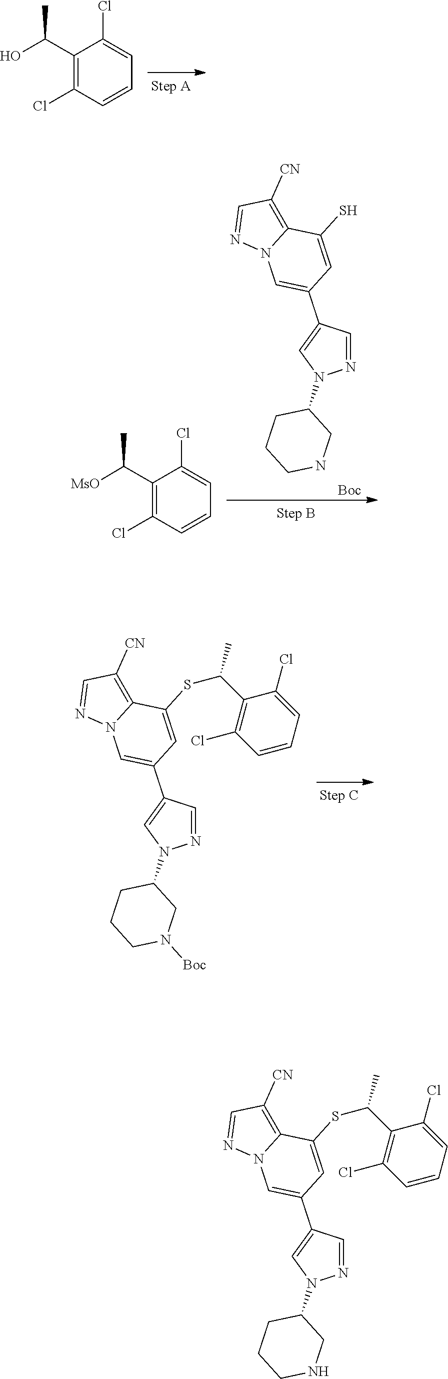 [SMILES: C[C@H](O)c1c(Cl)cccc1Cl ; C[C@H](OS(C)(=O)=O)c1c(Cl)cccc1Cl ; N#Cc1cnn2cc(-c3cnn([C@H]4CCCNC4)c3)cc(S)c12 ; C[C@@H](Sc1cc(-c2cnn([C@H]3CCCN(C(=O)OC(C)(C)C)C3)c2)cn2ncc(C#N)c12)c1c(Cl)cccc1Cl ; "Boc" ; C[C@@H](Sc1cc(-c2cnn([C@H]3CCCNC3)c2)cn2ncc(C#N)c12)c1c(Cl)cccc1Cl]

Step A. [(1S)-1-(2,6-Dichlorophenyl)ethyl] methanesulfonate. To a 8 mL vial equipped with a magnetic stir bar was added (1S)-1-(2,6-dichlorophenyl)ethanol (90 mg, 0.47 mmol) and DMAP (6.0 mg, 0.045 mmol) followed by the addition of DCM (2 mL). Triethylamine (0.20 mL, 1.4 mmol) and methylsulfonyl methanesulfonate (0.16 g, 0.94 mmol) were added to the mixture at 0° C. The mixture was stirred at 25° C. for 1 h. Water (5 mL) was added to the reaction and the mixture was transferred to a separatory funnel. The aqueous layer mixture was extracted with DCM (5 mL×3). The combined organic layers were washed with brine (20 mL), dried over anhydrous sodium sulfate, filtered, and concentrated under reduced pressure affording [(1S)-1-(2,6-dichlorophenyl)ethyl] methanesulfonate (0.12 g, 95% yield) as a yellow oil which was used in the next step without further purification.

Step B. t-Butyl (3S)-3-[4-[3-cyano-4-[(1R)-1-(2,6-dichlorophenyl)ethyl]sulfanyl-pyrazolo[1,5-a]pyridin-6-yl]pyrazol-1-yl]piperidine-1-carboxylate. Synthesized according to Example 10, Step A, substituting [(1S)-1-(2,6-dichlorophenyl)ethyl] methanesulfonate for [(1S)-1-(3,5-dichloro-4-pyridyl)ethyl] methanesulfonate to give tert-butyl (3S)-3-[4-[3-cyano-4-[(1R)-1-(2,6-dichlorophenyl)ethyl]sulfanyl-pyrazolo[1,5-a]pyridin-6-yl]pyrazol-1-yl]piperidine-1-carboxylate (0.13 g, 83% yield) as a yellow gum. LCMS (MM-ES+APCI, Pos): m/z 597.5 (M+H).

Step C. 4-[(1R)-1-(2,6-Dichlorophenyl)ethyl]sulfanyl-6-[1-[(3S)-3-piperidyl]pyrazol-4-yl]pyrazolo[1,5-a]pyridine-3-carbonitrile. Synthesized according to Example 1, Step B, substituting tert-butyl (3S)-3-[4-[3-cyano-4-[(1R)-1-(2,6-dichlorophenyl)ethyl]sulfanyl-pyrazolo[1,5-a]pyridin-6-yl]pyrazol-1-yl]piperidine-1-carboxylate for tert-butyl (3S)-3-[4-[3-cyano-4-(2-pyridylsulfanyl)pyrazolo[1,5-a]pyridin-6-yl]pyrazol-1-yl]piperidine-1-carboxylate to give 4-[(1R)-1-(2,6-dichlorophenyl)ethyl]sulfanyl-6-[1-[(3S)-3-piperidyl]pyrazol-4-yl]pyrazolo[1,5-a]pyridine-3-carbonitrile (54 mg, 55% yield) as a white solid. LCMS (MM-ES+APCI, Pos): m/z 497.1 (M+H).

### Example 21

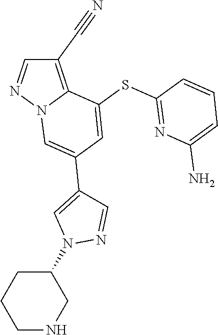 [SMILES: N#Cc1cnn2cc(-c3cnn([C@H]4CCCNC4)c3)cc(Sc3cccc(N)n3)c12]

### 4-[(6-Amino-2-pyridyl)sulfanyl]-6-[1-[(3S)-3-piperidyl]pyrazol-4-yl]pyrazolo[1,5-a]pyridine-3-carbonitrile (21)

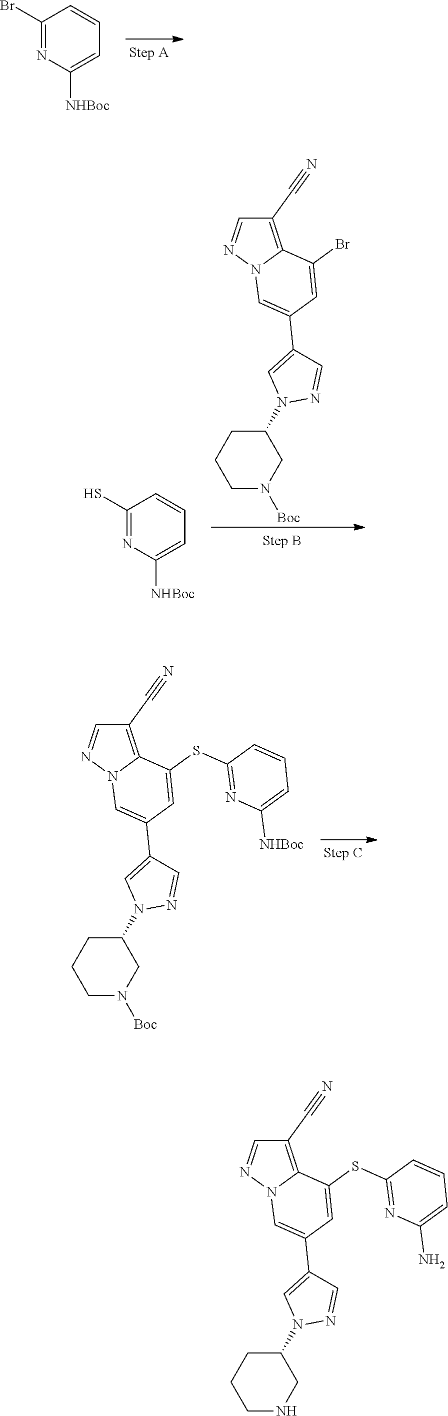 [SMILES: CC(C)(C)OC(=O)Nc1cccc(Br)n1 ; CC(C)(C)OC(=O)Nc1cccc(S)n1 ; CC(C)(C)OC(=O)N1CCC[C@H](n2cc(-c3cc(Br)c4c(C#N)cnn4c3)cn2)C1 ; CC(C)(C)OC(=O)Nc1cccc(Sc2cc(-c3cnn([C@H]4CCCN(C(=O)OC(C)(C)C)C4)c3)cn3ncc(C#N)c23)n1 ; N#Cc1cnn2cc(-c3cnn([C@H]4CCCNC4)c3)cc(Sc3cccc(N)n3)c12]

Step A. t-Butyl N-(6-sulfanyl-2-pyridyl)carbamate. To a 40 mL vial equipped with a magnetic stir bar was added tert-butyl N-(6-bromo-2-pyridyl) carbamate (0.90 g, 3.3 mmol) followed by the addition of toluene (10 mL). Triisopropyl(sulfanyl)silane (0.92 mL, 4.3 mmol), Pd(dppf)Cl₂ (0.12 g, 0.16 mmol) and Cs₂CO₃ (1.4 g, 4.3 mmol) were added into the mixture at 25° C. The vial was purged with nitrogen 3 min and stirred at 100° C. under an atmosphere of nitrogen for 15 h. Water (10 mL) was added to the reaction and the mixture was transferred to a separatory funnel. The aqueous layer was extracted with ethyl acetate (10 mL×3). The combined organic layers were washed with brine (15 mL), dried over anhydrous sodium sulfate, filtered, concentrated under reduced and purified by silica gel chromatography (40-50% ethyl acetate/petroleum ether) to give tert-butyl N-(6-sulfanyl-2-pyridyl)carbamate (0.68 g, 80% yield) as an orange solid. LCMS (MM-ES+APCI, Pos): m/z 171.0 (M+H).

Step B. t-Butyl (3S)-3-[4-[4-[[6-(tert-butoxycarbonylamino)-2-pyridyl]sulfanyl]-3-cyano-pyrazolo[1,5-a]pyridin-6-yl]pyrazol-1-yl]piperidine-1-carboxylate. Synthesized according to Example 3, Step C, substituting tert-butyl N-(6-sulfanyl-2-pyridyl) carbamate for 2-fluoropyridine-3-thiol to give tert-butyl (3S)-3-[4-[4-[[6-(tert-butoxycarbonylamino)-2-pyridyl]sulfanyl]-3-cyano-pyrazolo[1,5-a]pyridin-6-yl]pyrazol-1-yl]piperidine-1-carboxylate (0.26 g, 64% yield) as an orange solid. LCMS (MM-ES+APCI, Pos): m/z 517.8 (M+H).

Step C. 4-[(6-Amino-2-pyridyl)sulfanyl]-6-[1-[(3S)-3-piperidyl]pyrazol-4-yl]pyrazolo[1,5-a]pyridine-3-carbonitrile. Synthesized according to Example 1, Step B, substituting tert-butyl (3S)-3-[4-[4-[[6-(tert-butoxycarbonylamino)-2-pyridyl]sulfanyl]-3-cyano-pyrazolo[1,5-a]pyridin-6-yl]pyrazol-1-yl]piperidine-1-carboxylate for tert-butyl (3S)-3-[4-[3-cyano-4-(2-pyridylsulfanyl)pyrazolo[1,5-a]pyridin-6-yl]pyrazol-1-yl]piperidine-1-carboxylate to give 4-[(6-amino-2-pyridyl)sulfanyl]-6-[1-[(3S)-3-piperidyl]pyrazol-4-yl]pyrazolo[1,5-a]pyridine-3-carbonitrile (58 mg, 43% yield) as a white solid. LCMS (MM-ES+APCI, Pos): m/z 417.1 (M+H).

### Example 22

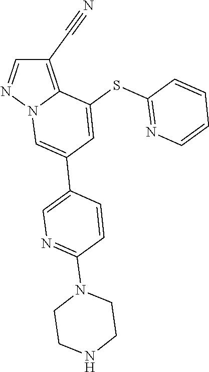 [SMILES: N#Cc1cnn2cc(-c3ccc(N4CCNCC4)nc3)cc(Sc3ccccn3)c12]

### 6-(6-Piperazin-1-yl-3-pyridyl)-4-(2-pyridylsulfanyl)pyrazolo[1,5-a]pyridine-3-carbonitrile (22)

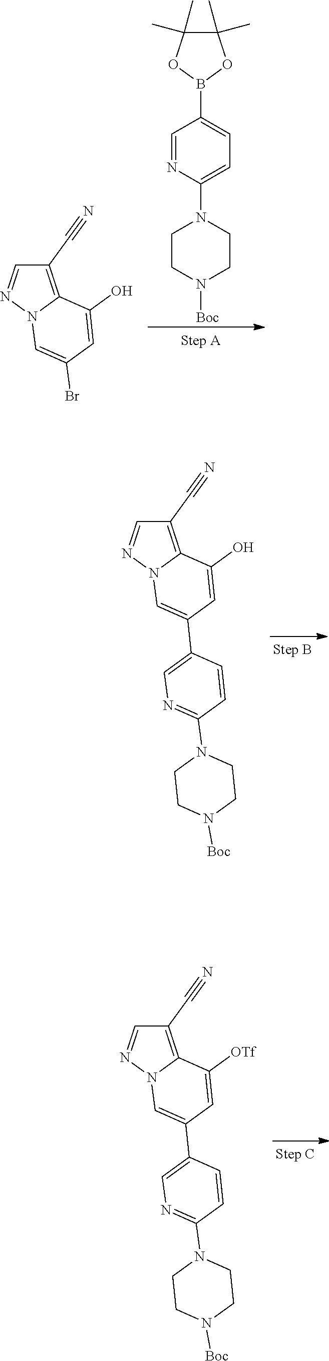

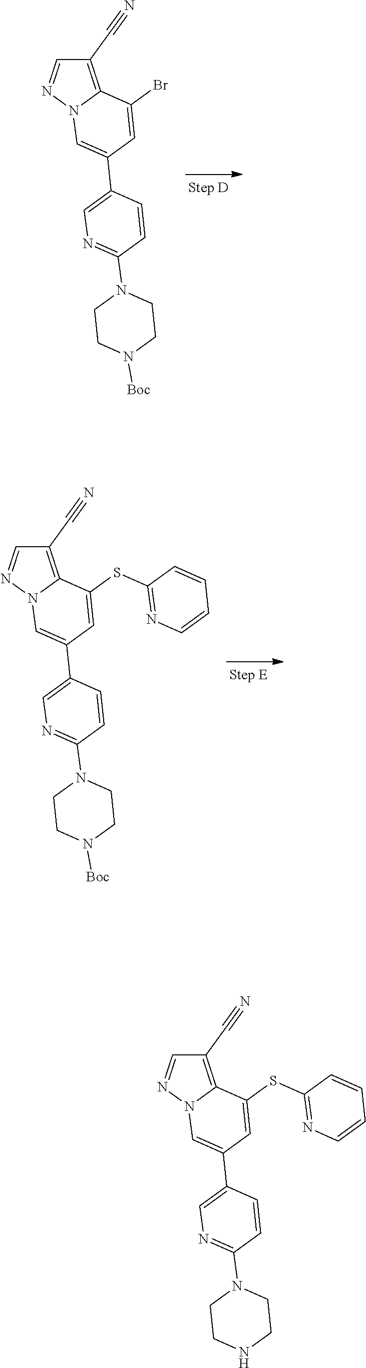 [SMILES: CC(C)(C)OC(=O)N1CCN(c2ccc(-c3cc(Br)c4c(C#N)cnn4c3)cn2)CC1 ; CC(C)(C)OC(=O)N1CCN(c2ccc(-c3cc(Sc4ccccn4)c4c(C#N)cnn4c3)cn2)CC1 ; N#Cc1cnn2cc(-c3ccc(N4CCNCC4)nc3)cc(Sc3ccccn3)c12]

Step A. t-Butyl 4-[5-(3-cyano-4-hydroxy-pyrazolo[1,5-a]pyridin-6-yl)-2-pyridyl]piperazine-1-carboxylate. Synthesized according to Intermediate 2, Step A, substituting tert-butyl 4-[5-(4,4,5,5-tetramethyl-1,3,2-dioxaborolan-2-yl)-2-pyridyl]piperazine-1-carboxylate for tert-butyl (3S)-3-[4-(4,4,5,5-tetramethyl-1,3,2-dioxaborolan-2-yl)pyrazol-1-yl]piperidine-1-carboxylate to give tert-butyl 4-[5-(3-cyano-4-hydroxy-pyrazolo[1,5-a]pyridin-6-yl)-2-pyridyl]piperazine-1-carboxylate (0.24 g, 36% yield) as a brown solid. LCMS (MM-ES+APCI, Pos): m/z 364.9 (M+H).

Step B. t-Butyl 4-[5-[3-cyano-4-(trifluoromethylsulfonyloxy)pyrazolo[1,5-a]pyridin-6-yl]-2-pyridyl]piperazine-1-carboxylate. Synthesized according to Intermediate 2, Step B, substituting tert-butyl 4-[5-(3-cyano-4-hydroxy-pyrazolo[1,5-a]pyridin-6-yl)-2-pyridyl]piperazine-1-carboxylate for tert-butyl (3S)-3-[4-(3-cyano-4-hydroxy-pyrazolo[1,5-a]pyridin-6-yl)pyrazol-1-yl]piperidine-1-carboxylate to give tert-butyl 4-[5-[3-cyano-4-(trifluoromethylsulfonyloxy)pyrazolo[1,5-a]pyridin-6-yl]-2-pyridyl]piperazine-1-carboxylate (0.59 g, 80% yield) as a gray solid. LCMS (MM-ES+APCI, Pos): m/z 553.1 (M+H).

Step C. t-Butyl 4-[5-(4-bromo-3-cyano-pyrazolo[1,5-a]pyridin-6-yl)-2-pyridyl]piperazine-1-carboxylate. Synthesized according to Intermediate 2, Step C, substituting tert-butyl 4-[5-[3-cyano-4-(trifluoromethylsulfonyloxy)pyrazolo[1,5-a]pyridin-6-yl]-2-pyridyl]piperazine-1-carboxylate for tert-butyl (3S)-3-[4-[3-cyano-4-(trifluoromethylsulfonyloxy)pyrazolo[1,5-a]pyridin-6-yl]pyrazol-1-yl]piperidine-1-carboxylate to give tert-butyl 4-[5-(4-bromo-3-cyano-pyrazolo[1,5-a]pyridin-6-yl)-2-pyridyl]piperazine-1-carboxylate (0.41 g, 88% yield,) as a brown solid. LCMS (MM-ES+APCI, Pos): m/z 485.0 (M+H).

Step D. t-Butyl 4-[5-[3-cyano-4-(2-pyridylsulfanyl)pyrazolo[1,5-a]pyridin-6-yl]-2-pyridyl]piperazine-1-carboxylate. Synthesized according to Example 1, Step A, substituting tert-butyl 4-[5-(4-bromo-3-cyano-pyrazolo[1,5-a]pyridin-6-yl)-2-pyridyl]piperazine-1-carboxylate for tert-butyl (3S)-3-[4-(4-bromo-3-cyano-pyrazolo[1,5-a]pyridin-6-yl)pyrazol-1-yl]piperidine-1-carboxylate to give tert-butyl 4-[5-[3-cyano-4-(2-pyridylsulfanyl)pyrazolo[1,5-a]pyridin-6-yl]-2-pyridyl]piperazine-1-carboxylate (0.22 g, 53% yield) as a yellow solid. LCMS (MM-ES+APCI, Pos): m/z 514.3 (M+H).

Step E. 6-(6-Piperazin-1-yl-3-pyridyl)-4-(2-pyridylsulfanyl)pyrazolo[1,5-a]pyridine-3-carbonitrile. Synthesized according to Example 1, Step B, substituting tert-butyl 4-[5-[3-cyano-4-(2-pyridylsulfanyl)pyrazolo[1,5-a]pyridin-6-yl]-2-pyridyl]piperazine-1-carboxylate for tert-butyl (3S)-3-[4-[3-cyano-4-(2-pyridylsulfanyl)pyrazolo[1,5-a]pyridin-6-yl]pyrazol-1-yl]piperidine-1-carboxylate to give 6-(6-piperazin-1-yl-3-pyridyl)-4-(2-pyridylsulfanyl)pyrazolo[1,5-a]pyridine-3-carbonitrile (50 mg, 33% yield) was obtained as an off-white solid. LCMS (MM-ES+APCI, Pos): m/z 414.3 (M+H).

### Example 23

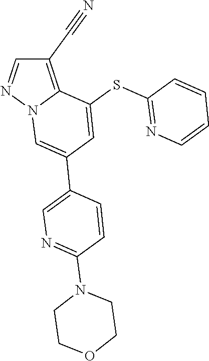 [SMILES: N#Cc1cnn2cc(-c3ccc(N4CCOCC4)nc3)cc(Sc3ccccn3)c12]

### 6-(6-Morpholino-3-pyridyl)-4-(2-pyridylsulfanyl)pyrazolo[1,5-a]pyridine-3-carbonitrile (23)

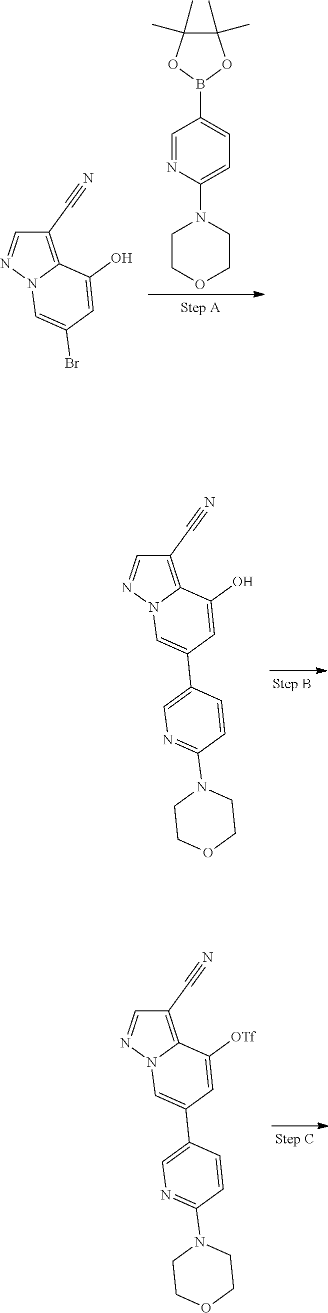

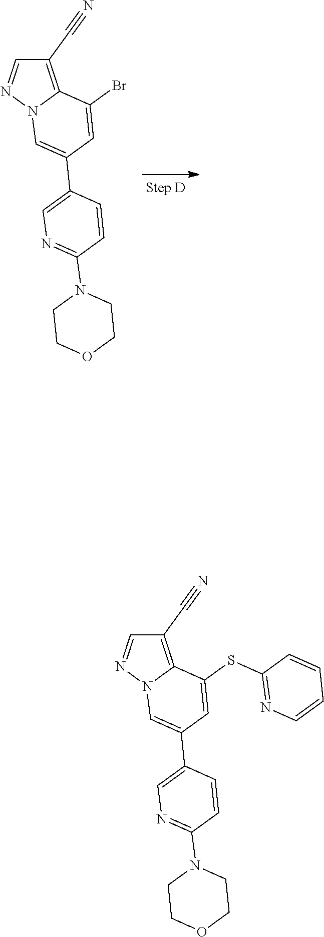 [SMILES: N#Cc1cnn2cc(-c3ccc(N4CCOCC4)nc3)cc(Br)c12 ; N#Cc1cnn2cc(-c3ccc(N4CCOCC4)nc3)cc(Sc3ccccn3)c12]

Step A. 4-hydroxy-6-(6-morpholino-3-pyridyl)pyrazolo[1,5-a]pyridine-3-carbonitrile.

Synthesized according to Intermediate 2, Step A, substituting 4-[5-(4,4,5,5-tetramethyl-1,3,2-dioxaborolan-2-yl)-2-pyridyl]morpholine for tert-butyl (3S)-3-[4-(4,4,5,5-tetramethyl-1,3,2-dioxaborolan-2-yl)pyrazol-1-yl]piperidine-1-carboxylate to give 4-hydroxy-6-(6-morpholino-3-pyridyl)pyrazolo[1,5-a]pyridine-3-carbonitrile (0.33 g, 58% yield) as a brown solid. LCMS (MM-ES+APCI, Pos): m/z 322.1 (M+H).

Step B. [3-Cyano-6-(6-morpholino-3-pyridyl)pyrazolo[1,5-a]pyridin-4-yl] trifluoromethanesulfonate. Synthesized according to Intermediate 2, Step B, substituting 4-hydroxy-6-(6-morpholino-3-pyridyl)pyrazolo[1,5-a]pyridine-3-carbonitrile for tert-butyl (3S)-3-[4-(3-cyano-4-hydroxy-pyrazolo[1,5-a]pyridin-6-yl)pyrazol-1-yl]piperidine-1-carboxylate to give [3-cyano-6-(6-morpholino-3-pyridyl)pyrazolo[1,5-a]pyridin-4-yl] trifluoromethanesulfonate (0.3 g, 93% yield) as a yellow solid. LCMS (MM-ES+APCI, Pos): m/z 454.0 (M+H).

Step C. 4-Bromo-6-(6-morpholino-3-pyridyl)pyrazolo[1,5-a]pyridine-3-carbonitrile. Synthesized according to Intermediate 2, Step C, substituting [3-cyano-6-(6-morpholino-3-pyridyl)pyrazolo[1,5-a]pyridin-4-yl]trifluoromethanesulfonate for tert-butyl (3S)-3-[4-[3-cyano-4-(trifluoromethylsulfonyloxy)pyrazolo[1,5-a]pyridin-6-yl]pyrazol-1-yl]piperidine-1-carboxylate to give 4-bromo-6-(6-morpholino-3-pyridyl)pyrazolo[1,5-a]pyridine-3-carbonitrile (0.21 g, 69% yield,) as a brown solid. LCMS (MM-ES+APCI, Pos): m/z 384.2 (M+H).

Step D. t-Butyl 4-[5-[3-cyano-4-(2-pyridylsulfanyl)pyrazolo[1,5-a]pyridin-6-yl]-2-pyridyl]piperazine-1-carboxylate. Synthesized according to Example 1, Step A, substituting tert-butyl (3S)-3-[4-(4-bromo-3-cyano-pyrazolo[1,5-a]pyridin-6-yl)pyrazol-1-yl]piperidine-1-carboxylate with 4-bromo-6-(6-morpholino-3-pyridyl)pyrazolo[1,5-a]pyridine-3-carbonitrile to give 6-(6-morpholino-3-pyridyl)-4-(2-pyridylsulfanyl)pyrazolo[1,5-a]pyridine-3-carbonitrile (52 mg, 35% yield) as a yellow solid. LCMS (MM-ES+APCI, Pos): m/z 414.9 (M+H).

### Example 24

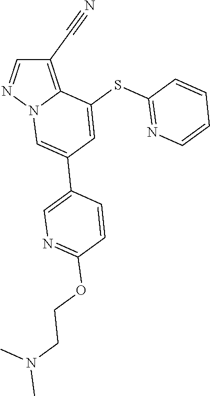 [SMILES: CN(C)CCOc1ccc(-c2cc(Sc3ccccn3)c3c(C#N)cnn3c2)cn1]

### 6-[6-[2-(Dimethylamino)ethoxy]-3-pyridyl]-4-(2-pyridylsulfanyl)pyrazolo[1,5-a]pyridine-3-carbonitrile (24)

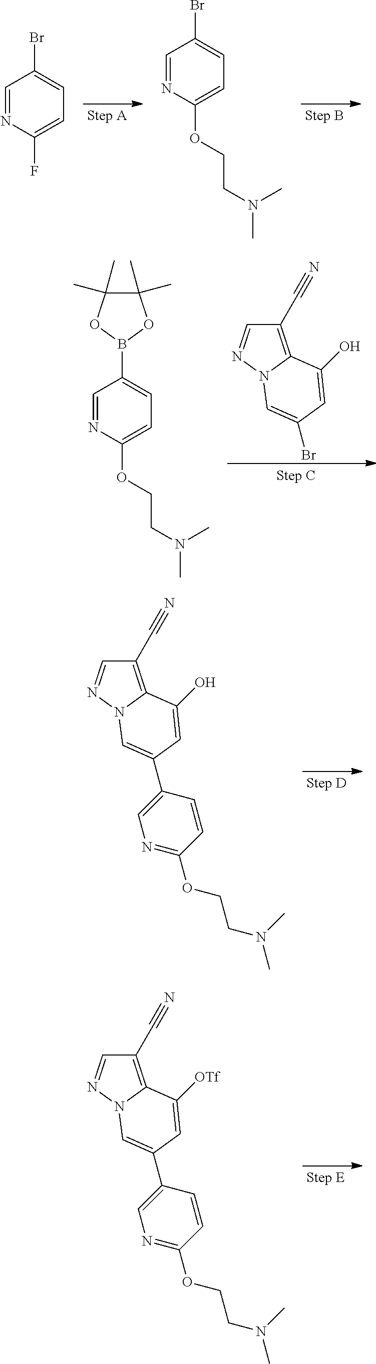

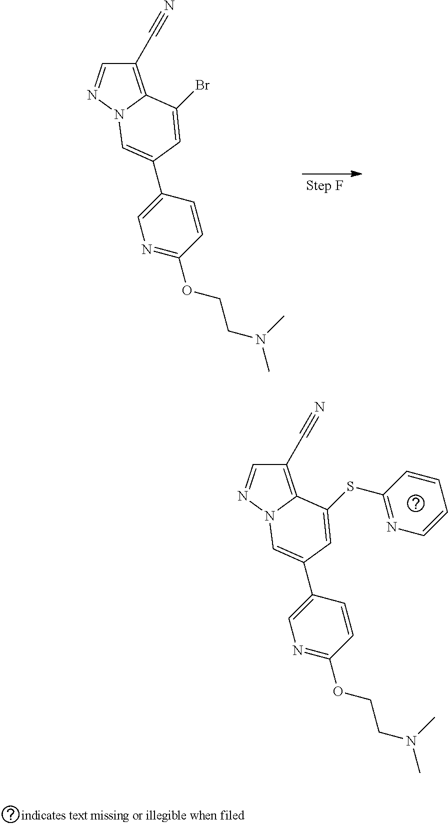 [SMILES: CN(C)CCOc1ccc(-c2cc(Br)c3c(C#N)cnn3c2)cn1 ; CN(C)CCOc1ccc(-c2cc(Sc3ccccn3)c3c(C#N)cnn3c2)cn1]

Step A. 2-[(5-Bromo-2-pyridyl)oxy]-N,N-dimethyl-ethanamine. To a 40 mL vial flask was added 5-bromo-2-fluoro-pyridine (1.0 g, 5.7 mmol) and 2-(dimethylamino)ethanol (0.51 g, 5.7 mmol) followed by the addition of DMF (15 mL). Cs₂CO₃ (3.7 g, 11 mmol) was added into the mixture. The flask was purged with nitrogen three times and stirred at 60° C. under an atmosphere of nitrogen for 16 h. Saturated aqueous ammonium chloride (30 mL) was added to the reaction and the mixture was transferred to a separatory funnel. The aqueous layer was extracted with ethyl acetate (20 mL×3). The combined organic layers were washed with brine (20 mL), dried over anhydrous sodium sulfate, filtered, concentrated under reduced pressure and purified by silica gel chromatography (0-10% Dichloromethane/Methanol) to give 2-[(5-bromo-2-pyridyl)oxy]-N,N-dimethyl-ethanamine (2.5 g, 90% yield) as a yellow oil. LCMS (MM-ES+APCI, Pos): m/z 246.9 (M+H).

Step B. N,N-Dimethyl-2-[[5-(4,4,5,5-tetramethyl-1,3,2-dioxaborolan-2-yl)-2-pyridyl]oxy]ethanamine. To a 50 mL round-bottom flask was added 2-[(5-bromo-2-pyridyl)oxy]-N,N-dimethylethanamine (0.50 g, 2.0 mmol) and 4,4,5,5-tetramethyl-2-(4,4,5,5-tetramethyl-1,3,2-dioxaborolan-2-yl)-1,3,2-dioxaborolane (0.78 g, 3.1 mmol) followed by the addition of dioxane (15 mL). KOAc (0.60 g, 6.1 mmol) and Pd(dppf)Cl₂ (0.15 g, 0.20 mmol) were added into the mixture. The flask was purged with nitrogen three times. The mixture was stirred at 90° C. under an atmosphere of nitrogen for 1 h. The suspension was filtered through a pad of Celite and the Celite washed with ethyl acetate (50 mL). The filtrate was concentrated under reduced pressure to give the crude product N,N-dimethyl-2-[[5-(4,4,5,5-tetramethyl-1,3,2-dioxaborolan-2-yl)-2-pyridyl]oxy]ethanamine (1.2 g) as black brown gum which was used in the next step without further purification.

Step C. 6-[6-[2-(Dimethylamino)ethoxy]-3-pyridyl]-4-hydroxy-pyrazolo[1,5-a]pyridine-3-carbonitrile. Synthesized according to Intermediate 2, Step A, substituting N,N-dimethyl-2-[[5-(4,4,5,5-tetramethyl-1,3,2-dioxaborolan-2-yl)-2-pyridyl]oxy]ethanamine for tert-butyl (3S)-3-[4-(4,4,5,5-tetramethyl-1,3,2-dioxaborolan-2-yl)pyrazol-1-yl]piperidine-1-carboxylate to give 6-[6-[2-(dimethylamino)ethoxy]-3-pyridyl]-4-hydroxy-pyrazolo[1,5-a] pyridine-3-carbonitrile (0.80 g, 87% yield) as a brown gum. LCMS (MM-ES+APCI, Pos): m/z 324.2 (M+H).

Step D. [3-Cyano-6-[6-[2-(dimethylamino)ethoxy]-3-pyridyl]pyrazolo[1,5-a]pyridin-4-yl]trifluoromethanesulfonate. Synthesized according to Intermediate 2, Step B, substituting 6-[6-[2-(dimethylamino)ethoxy]-3-pyridyl]-4-hydroxypyrazolo[1,5-a]pyridine-3-carbonitrile for tert-butyl (3S)-3-[4-(3-cyano-4-hydroxy-pyrazolo[1,5-a]pyridin-6-yl)pyrazol-1-yl]piperidine-1-carboxylate to give [3-cyano-6-[6-[2-(dimethylamino)ethoxy]-3-pyridyl]pyrazolo[1,5-a]pyridin-4-yl]trifluoromethanesulfonate (0.40 g, crude) as a brown gum which was used in the next step without further purification.

Step E. 4-Bromo-6-[6-[2-(dimethylamino)ethoxy]-3-pyridyl]pyrazolo[1,5-a]pyridine-3-carbonitrile. Synthesized according to Intermediate 2, Step C, substituting [3-cyano-6-[6-[2-(dimethylamino)ethoxy]-3-pyridyl]pyrazolo[1,5-a]pyridin-4-yl] trifluoromethanesulfonate for tert-butyl (3S)-3-[4-[3-cyano-4-(trifluoromethylsulfonyloxy)pyrazolo[1,5-a]pyridin-6-yl]pyrazol-1-yl]piperidine-1-carboxylate to give 4-bromo-6-[6-[2-(dimethylamino)ethoxy]-3-pyridyl]pyrazolo[1,5-a]pyridine-3-carbonitrile (0.10 g, 24% yield) as a yellow gum. LCMS (MM-ES+APCI, Pos): m/z 386.1 (M+H).

Step F. 6-[6-[2-(Dimethylamino)ethoxy]-3-pyridyl]-4-(2-pyridylsulfanyl)pyrazolo[1,5-a]pyridine-3-carbonitrile. Synthesized according to Example 1, Step A, substituting 4-bromo-6-[6-[2-(dimethylamino)ethoxy]-3-pyridyl]pyrazolo[1,5-a]pyridine-3-carbonitrile for tert-butyl (3S)-3-[4-(4-bromo-3-cyano-pyrazolo[1,5-a]pyridin-6-yl)pyrazol-1-yl]piperidine-1-carboxylate to give 6-[6-[2-(dimethylamino)ethoxy]-3-pyridyl]-4-(2-pyridylsulfanyl)pyrazolo[1,5-a]pyridine-3-carbonitrile (21 mg, 24% yield) as an off-white solid. LCMS (MM-ES+APCI, Pos): m/z 417.3 (M+H).

### Example 25

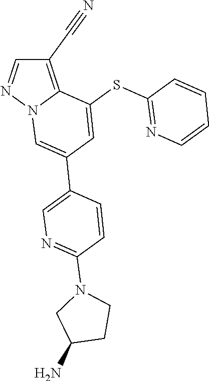 [SMILES: N#Cc1cnn2cc(-c3ccc(N4CC[C@@H](N)C4)nc3)cc(Sc3ccccn3)c12]

### 6-[6-[(3R)-3-Aminopyrrolidin-1-yl]-3-pyridyl]-4-(2-pyridylsulfanyl)pyrazolo[1,5-a]pyridine-3-carbonitrile (25)

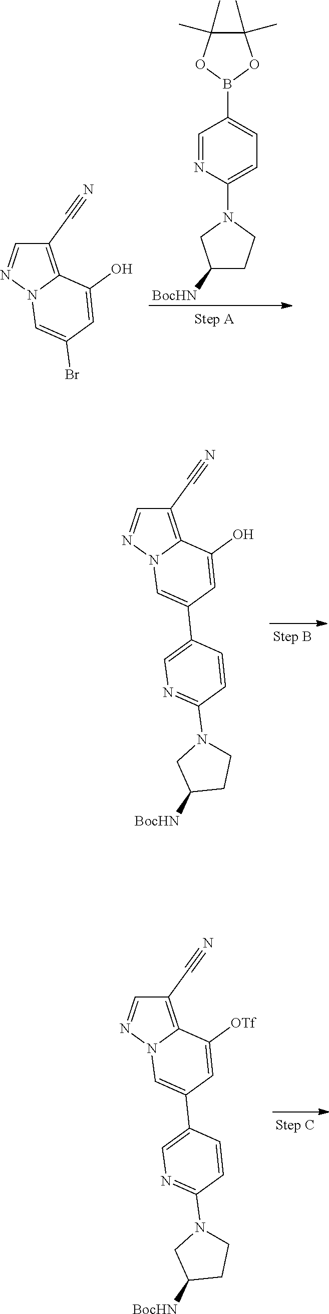

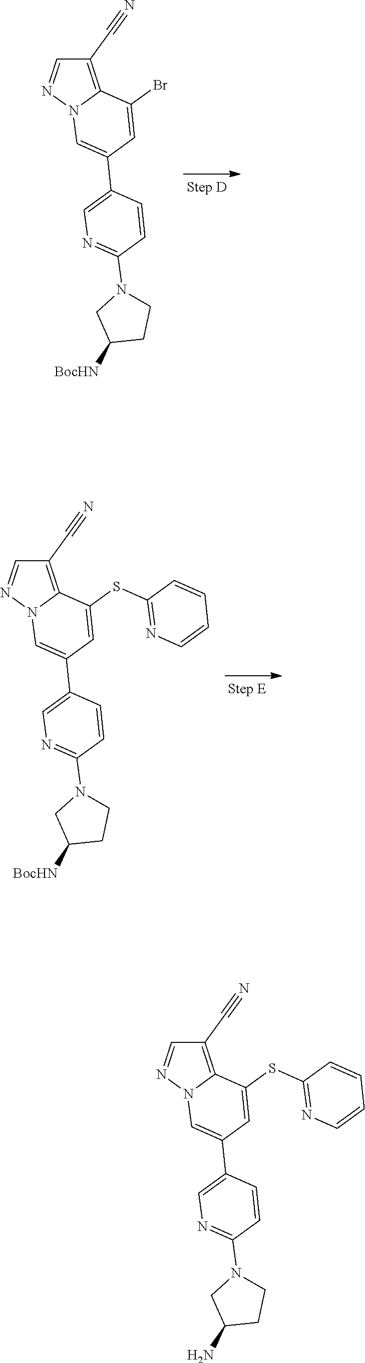 [SMILES: CC(C)(C)OC(=O)N[C@@H]1CCN(c2ccc(-c3cc(Br)c4c(C#N)cnn4c3)cn2)C1 ; CC(C)(C)OC(=O)N[C@@H]1CCN(c2ccc(-c3cc(Sc4ccccn4)c4c(C#N)cnn4c3)cn2)C1 ; N#Cc1cnn2cc(-c3ccc(N4CC[C@@H](N)C4)nc3)cc(Sc3ccccn3)c12]

Step A. t-Butyl N-[(3R)-1-[5-(3-cyano-4-hydroxy-pyrazolo[1,5-a]pyridin-6-yl)-2-pyridyl]pyrrolidin-3-yl]carbamate. Synthesized according to Intermediate 2, Step A, substituting tert-butyl N-[(3R)-1-[5-(4,4,5,5-tetramethyl-1,3,2-dioxaborolan-2-yl)-2-pyridyl]pyrrolidin-3-yl]carbamate for tert-butyl (3S)-3-[4-(4,4,5,5-tetramethyl-1,3,2-dioxaborolan-2-yl)pyrazol-1-yl]piperidine-1-carboxylate to give tert-butyl N-[(3R)-1-[5-(3-cyano-4-hydroxy-pyrazolo[1,5-a]pyridin-6-yl)-2-pyridyl]pyrrolidin-3-yl]carbamate (0.30 g, 46% yield) as a light solid. LCMS (MM-ES+APCI, Pos): m/z 421.2 (M+H).

Step B. [6-[6-[(3R)-3-(t-Butoxycarbonylamino)pyrrolidin-1-yl]-3-pyridyl]-3-cyano-pyrazolo[1,5-a]pyridin-4-yl] trifluoromethanesulfonate. Synthesized according to Intermediate 2, Step B, substituting tert-butyl N-[(3R)-1-[5-(3-cyano-4-hydroxy-pyrazolo[1,5-a]pyridin-6-yl)-2-pyridyl] pyrrolidin-3-yl] carbamate for tert-butyl (3S)-3-[4-(3-cyano-4-hydroxy-pyrazolo[1,5-a]pyridin-6-yl)pyrazol-1-yl]piperidine-1-carboxylate to give [6-[6-[(3R)-3-(tert-butoxycarbonylamino)pyrrolidin-1-yl]-3-pyridyl]-3-cyano-pyrazolo[1,5-a]pyridin-4-yl] trifluoromethanesulfonate (0.32 g, 88% yield) as a yellow solid. LCMS (MM-ES+APCI, Pos): m/z 553.3 (M+H).

Step C. t-Butyl N-[(3R)-1-[5-(4-bromo-3-cyano-pyrazolo[1,5-a]pyridin-6-yl)-2-pyridyl]pyrrolidin-3-yl]carbamate. Synthesized according to Intermediate 2, Step C, substituting [6-[6-[(3R)-3-(tert-butoxycarbonylamino) pyrrolidin-1-yl]-3-pyridyl]-3-cyano-pyrazolo[1,5-a]pyridin-4-yl] trifluoromethanesulfonate for tert-butyl (3S)-3-[4-[3-cyano-4-(trifluoromethylsulfonyloxy)pyrazolo[1,5-a]pyridin-6-yl]pyrazol-1-yl]piperidine-1-carboxylate to give tert-butyl N-[(3R)-1-[5-(4-bromo-3-cyano-pyrazolo[1,5-a]pyridin-6-yl)-2-pyridyl]pyrrolidin-3-yl]carbamate (0.20 g, 74% yield) as a brown solid. LCMS (MM-ES+APCI, Pos): m/z 485.1 (M+H).

Step D. t-Butyl N-[(3R)-1-[5-[3-cyano-4-(2-pyridylsulfanyl)pyrazolo[1,5-a]pyridin-6-yl]-2-pyridyl]pyrrolidin-3-yl]carbamate. Synthesized according to Example 1, Step A, substituting tert-butyl N-[(3R)-1-[5-(4-bromo-3-cyano-pyrazolo[1,5-a]pyridin-6-yl)-2-pyridyl]pyrrolidin-3-yl]carbamate for tert-butyl (3S)-3-[4-(4-bromo-3-cyano-pyrazolo[1,5-a]pyridin-6-yl)pyrazol-1-yl]piperidine-1-carboxylate to give tert-butyl N-[(3R)-1-[5-[3-cyano-4-(2-pyridylsulfanyl)pyrazolo[1,5-a]pyridin-6-yl]-2-pyridyl]pyrrolidin-3-yl]carbamate (0.18 g, crude) obtained as a brown solid.

Step E. 6-[6-[(3R)-3-Aminopyrrolidin-1-yl]-3-pyridyl]-4-(2-pyridylsulfanyl)pyrazolo[1,5-a]pyridine-3-carbonitrile. Synthesized according to Example 1, Step B, substituting tert-butyl N-[(3R)-1-[5-[3-cyano-4-(2-pyridylsulfanyl)pyrazolo[1,5-a]pyridin-6-yl]-2-pyridyl]pyrrolidin-3-yl]carbamate for tert-butyl (3S)-3-[4-[3-cyano-4-(2-pyridylsulfanyl)pyrazolo[1,5-a]pyridin-6-yl]pyrazol-1-yl]piperidine-1-carboxylate to give 6-[6-[(3R)-3-aminopyrrolidin-1-yl]-3-pyridyl]-4-(2-pyridylsulfanyl)pyrazolo[1,5-a]pyridine-3-carbonitrile (46 mg, 36% yield) as a yellow solid. LCMS (MM-ES+APCI, Pos): m/z 414.2 (M+H).

### Example 26

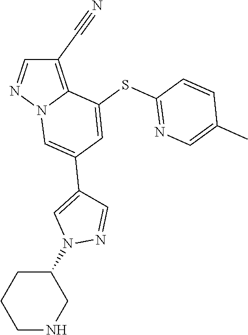 [SMILES: Cc1ccc(Sc2cc(-c3cnn([C@H]4CCCNC4)c3)cn3ncc(C#N)c23)nc1]

### 4-[(5-Methyl-2-pyridyl)sulfanyl]-6-[1-[(3S)-3-piperidyl]pyrazol-4-yl]pyrazolo[1,5-a]pyridine-3-carbonitrile (26)

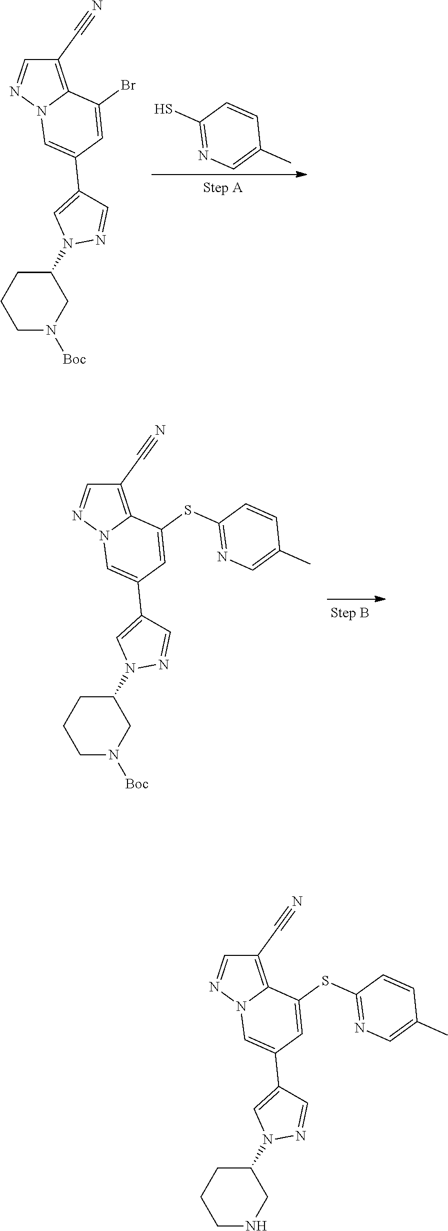

Step A. t-Butyl (3S)-3-[4-[3-cyano-4-[(5-methyl-2-pyridyl)sulfanyl]pyrazolo[1,5-a]pyridin-6-yl]pyrazol-1-yl]piperidine-1-carboxylate. To a 40 mL vial equipped with a magnetic stir bar was added tert-butyl (3S)-3-[4-(4-bromo-3-cyano-pyrazolo[1,5-a]pyridin-6-yl)pyrazol-1-yl]piperidine-1-carboxylate (0.18 g, 0.38 mmol) followed by the addition of DMF (4 mL). 5-methylpyridine-2-thiol (74 mg, 0.57 mmol), Cs₂CO₃ (0.25 g, 0.76 mmol), 1,10-phenanthroline (7.0 mg, 0.040 mmol) and copper iodide (7.0 mg, 0.034 mmol) were added to the mixture at 25° C. The flask was purged with nitrogen for 2 min and stirred at 100° C. under an atmosphere of nitrogen for 2 h. Water (10 mL) was added to the reaction and the mixture was transferred to a separatory funnel. The aqueous layer was extracted with ethyl acetate (10 mL×3). The combined organic layer was washed with brine (35 mL), dried over anhydrous sodium sulfate, filtered, and concentrated and purified by silica gel chromatography (50-60% EtOAc/petroleum ether) to give tert-butyl (3S)-3-[4-[3-cyano-4-[(5-methyl-2-pyridyl)sulfanyl]pyrazolo[1,5-a]pyridin-6-yl]pyrazol-1-yl]piperidine-1-carboxylate (60 mg, 30% yield) as a white solid. LCMS (MM-ES+APCI, Pos): m/z 516.3 (M+H).

Step B. 4-[(5-Methyl-2-pyridyl)sulfanyl]-6-[1-[(3S)-3-piperidyl]pyrazol-4-yl]pyrazolo[1,5-a]pyridine-3-carbonitrile. Synthesized according to Example 1, Step B, substituting tert-butyl (3S)-3-[4-[3-cyano-4-[(5-methyl-2-pyridyl)sulfanyl]pyrazolo[1,5-a]pyridin-6-yl]pyrazol-1-yl]piperidine-1-carboxylate for tert-butyl (3S)-3-[4-[3-cyano-4-(2-pyridylsulfanyl)pyrazolo[1,5-a]pyridin-6-yl]pyrazol-1-yl]piperidine-1-carboxylate to give 4-[(5-methyl-2-pyridyl)sulfanyl]-6-[1-[(3S)-3-piperidyl]pyrazol-4-yl]pyrazolo[1,5-a]pyridine-3-carbonitrile (26 mg, 65% yield) as a yellow solid. LCMS (MM-ES+APCI, Pos): m/z 416.0 (M+H).

### Example 27

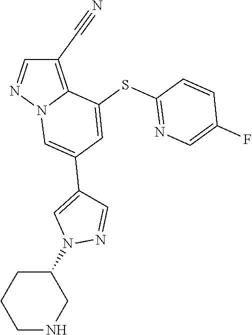 [SMILES: N#Cc1cnn2cc(-c3cnn([C@H]4CCCNC4)c3)cc(Sc3ccc(F)cn3)c12]

### 4-[(5-Fluoro-2-pyridyl)sulfanyl]-6-[1-[(3S)-3-piperidyl]pyrazol-4-yl]pyrazolo[1,5-a]pyridine-3-carbonitrile (27)

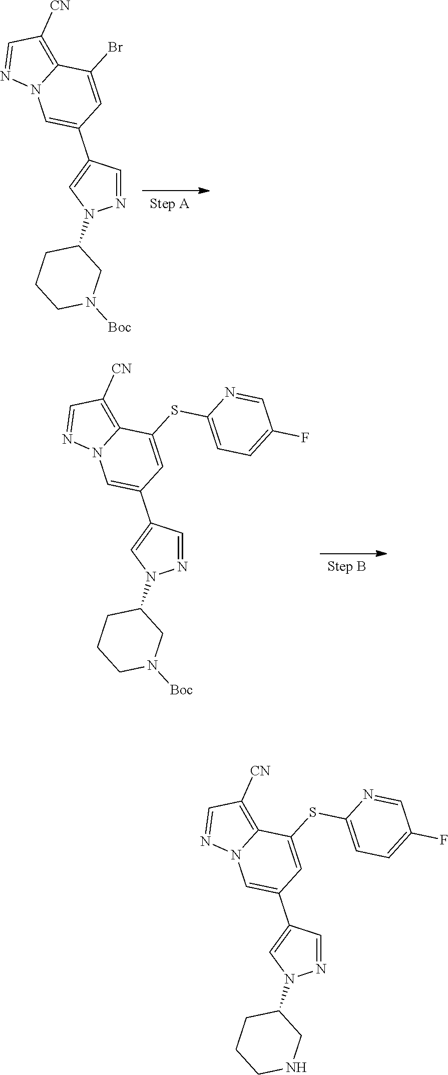

Step A. (S)-4-((5-Fluoropyridin-2-yl)thio)-6-(1-(piperidin-3-yl)-1H-pyrazol-4-yl)pyrazolo[1,5-a]pyridine-3-carbonitrile. Synthesized according to Example 25, Step A, substituting 5-fluoropyridine-2-thiol for 5-methylpyridine-2-thiol to give (S)-4-((5-fluoropyridin-2-yl)thio)-6-(1-(piperidin-3-yl)-1H-pyrazol-4-yl)pyrazolo[1,5-a]pyridine-3-carbonitrile (0.13 g, 72% yield) as a yellow solid. LCMS (MM-ES+APCI, Pos): m/z 463.9 (M-tBu+H).

Step B. 4-[(5-Fluoro-2-pyridyl)sulfanyl]-6-[1-[(3S)-3-piperidyl]pyrazol-4-yl]pyrazolo[1,5-a]pyridine-3-carbonitrile. Synthesized according to Example 1, Step B, substituting tert-butyl (3S)-3-[4-[3-cyano-4-[(5-fluoro-2-pyridyl)sulfanyl]pyrazolo[1,5-a]pyridin-6-yl]pyrazol-1-yl]piperidine-1-carboxylate for tert-butyl (3S)-3-[4-[3-cyano-4-(2-pyridylsulfanyl)pyrazolo[1,5-a]pyridin-6-yl]pyrazol-1-yl]piperidine-1-carboxylate to give 4-[(5-fluoro-2-pyridyl)sulfanyl]-6-[1-[(3S)-3-piperidyl]pyrazol-4-yl]pyrazolo[1,5-a]pyridine-3-carbonitrile (32 mg, 34% yield) as an off-white solid. LCMS (MM-ES+APCI, Pos): m/z 420.0 (M-tBu+H).

### Example 28

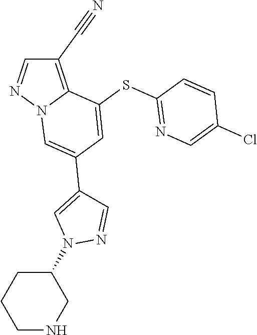 [SMILES: N#Cc1cnn2cc(-c3cnn([C@H]4CCCNC4)c3)cc(Sc3ccc(Cl)cn3)c12]

### 4-[(5-Chloro-2-pyridyl)sulfanyl]-6-[1-[(3S)-3-piperidyl]pyrazol-4-yl]pyrazolo[1,5-a]pyridine-3-carbonitrile (28)

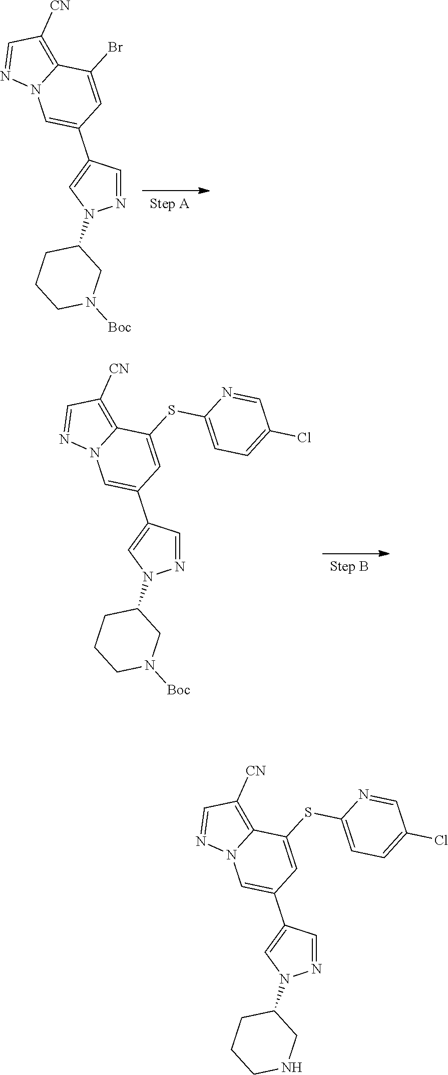

Step A. t-Butyl (3S)-3-[4-[4-[(5-chloro-2-pyridyl)sulfanyl]-3-cyano-pyrazolo[1,5-a]pyridin-6-yl]pyrazol-1-yl]piperidine-1-carboxylate. Synthesized according to Example 25, Step A, substituting 5-chloropyridine-2-thiol for 5-methylpyridine-2-thiol to give tert-butyl (3S)-3-[4-[4-[(5-chloro-2-pyridyl)sulfanyl]-3-cyano-pyrazolo[1,5-a]pyridin-6-yl]pyrazol-1-yl]piperidine-1-carboxylate (99 mg, 46% yield) as a yellow solid. LCMS (MM-ES+APCI, Pos): m/z 536.1 (M+H).

Step B. 4-[(5-Chloro-2-pyridyl)sulfanyl]-6-[1-[(3S)-3-piperidyl]pyrazol-4-yl]pyrazolo[1,5-a]pyridine-3-carbonitrile. Synthesized according to Example 1, Step B, substituting (3S)-3-[4-[4-[(5-chloro-2-pyridyl)sulfanyl]-3-cyano-pyrazolo[1,5-a]pyridin-6-yl]pyrazol-1-yl]piperidine-1-carboxylate for tert-butyl (3S)-3-[4-[3-cyano-4-(2-pyridylsulfanyl)pyrazolo[1,5-a]pyridin-6-yl]pyrazol-1-yl]piperidine-1-carboxylate to give 4-[(5-chloro-2-pyridyl)sulfanyl]-6-[1-[(3S)-3-piperidyl]pyrazol-4-yl]pyrazolo[1,5-a]pyridine-3-carbonitrile (34 mg, 54% yield) as an off-white solid. LCMS (MM-ES+APCI, Pos): m/z 436.2 (M+H).

### Example 29

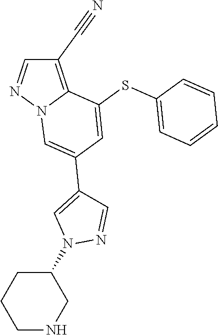 [SMILES: N#Cc1cnn2cc(-c3cnn([C@H]4CCCNC4)c3)cc(Sc3ccccc3)c12]

### 4-Phenylsulfanyl-6-[1-[(3S)-3-piperidyl]pyrazol-4-yl]pyrazolo[1,5-a]pyridine-3-carbonitrile (29)

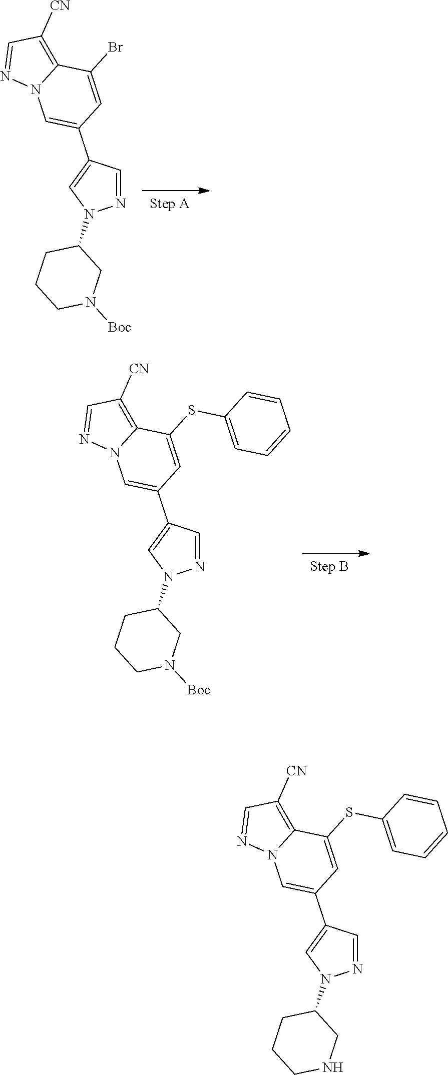

Step A. t-Butyl (3S)-3-[4-(3-cyano-4-phenylsulfanyl-pyrazolo[1,5-a]pyridin-6-yl)pyrazol-1-yl]piperidine-1-carboxylate. Synthesized according to Example 3, Step C, substituting benzenethiol for 2-fluoropyridine-3-thiol to give tert-butyl (3S)-3-[4-(3-cyano-4-phenylsulfanyl-pyrazolo[1,5-a]pyridin-6-yl)pyrazol-1-yl]piperidine-1-carboxylate (0.20 g, crude) as a black brown oil. LCMS (MM-ES+APCI, Pos): m/z 501.0 (M+H).

Step B. 4-Phenylsulfanyl-6-[1-[(3S)-3-piperidyl]pyrazol-4-yl]pyrazolo[1,5-a]pyridine-3-carbonitrile. Synthesized according to Example 11, Step B, substituting tert-butyl (3S)-3-[4-(3-cyano-4-phenylsulfanyl-pyrazolo[1,5-a]pyridin-6-yl)pyrazol-1-yl]piperidine-1-carboxylate for 2-fluoropyridine-3-thiol to give 4-phenylsulfanyl-6-[1-[(3S)-3-piperidyl]pyrazol-4-yl]pyrazolo[1,5-a]pyridine-3-carbonitrile (30 mg, 23% yield) as an off-white solid. LCMS (MM-ES+APCI, Pos): m/z 401.2 (M+H).

### Example 30

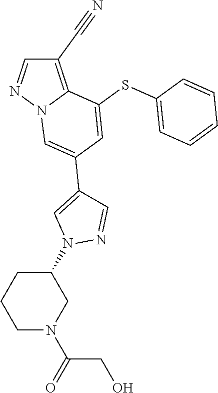 [SMILES: N#Cc1cnn2cc(-c3cnn([C@H]4CCCN(C(=O)CO)C4)c3)cc(Sc3ccccc3)c12]

### 6-[1-[(3S)-1-(2-Hydroxyacetyl)-3-piperidyl]pyrazol-4-yl]-4-(2-pyridylsulfanyl)pyrazolo[1,5-a]pyridine-3-carbonitrile (30)

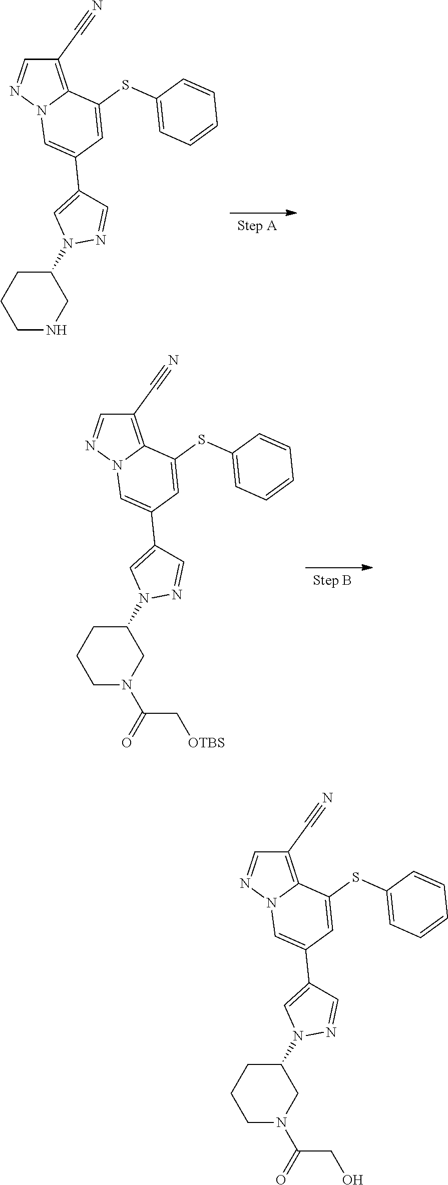 [SMILES: N#Cc1cnn2cc(-c3cnn([C@H]4CCCNC4)c3)cc(Sc3ccccc3)c12 ; CC(C)(C)[Si](C)(C)OCC(=O)N1CCC[C@H](n2cc(-c3cc(Sc4ccccc4)c4c(C#N)cnn4c3)cn2)C1 ; N#Cc1cnn2cc(-c3cnn([C@H]4CCCN(C(=O)CO)C4)c3)cc(Sc3ccccc3)c12]

Step A. 6-[1-[(3S)-1-[2-[t-Butyl(dimethyl)silyl]oxyacetyl]-3-piperidyl] pyrazol-4-yl]-4-(2-pyridylsulfanyl), pyrazolo[1,5-a]pyridine-3-carbonitrile. To a 8 mL vial equipped with a magnetic stir bar was added 2-[tert-butyl(dimethyl)silyl]oxyacetic acid (72 mg, 0.38 mmol) followed by the addition of DMF (1 mL). HATU (0.14 g, 0.37 mmol), DIEA (0.13 mL, 0.75 mmol) and 6-[1-[(3S)-3-piperidyl]pyrazol-4-yl]-4-(2-pyridylsulfanyl)pyrazolo[1,5-a]pyridine-3-carbonitrile (0.11 g, 0.25 mmol) were and stirred at 25° C. for 1 h. Water (10 mL) was added to the reaction and the mixture was transferred to a separatory funnel. The aqueous layer was extracted with ethyl acetate (10 mL×3). The combined organic layers were washed with brine (35 mL), dried over anhydrous sodium sulfate, filtered, and purified by silica gel chromatography (50-100% EtOAc/petroleum ether) to give 6-[1-[(3S)-1-[2-[tert-butyl(dimethyl)silyl]oxyacetyl]-3-piperidyl] pyrazol-4-yl]-4-(2-pyridylsulfanyl), pyrazolo[1,5-a]pyridine-3-carbonitrile (0.14 mg, 91% yield) as a white solid. LCMS (MM-ES+APCI, Pos): m/z 574.2 (M+H).

Step B. 6-[1-[(3S)-1-(2-Hydroxyacetyl)-3-piperidyl]pyrazol-4-yl]-4-(2-pyridylsulfanyl)pyrazolo[1,5-a]pyridine-3-carbonitrile. Synthesized according to Example 1, Step B, substituting 6-[1-[(3S)-1-[2-[tert-butyl(dimethyl)silyl]oxyacetyl]-3-piperidyl]pyrazol-4-yl]-4-(2-pyridylsulfanyl)pyrazolo[1,5-a]pyridine-3-carbonitrile for tert-butyl (3S)-3-[4-[3-cyano-4-(2-pyridylsulfanyl)pyrazolo[1,5-a]pyridin-6-yl]pyrazol-1-yl]piperidine-1-carboxylate to give 6-[1-[(3S)-1-(2-hydroxyacetyl)-3-piperidyl]pyrazol-4-yl]-4-(2-pyridylsulfanyl)pyrazolo[1,5-a]pyridine-3-carbonitrile (32 mg, 30% yield) as an off-white solid. LCMS (MM-ES+APCI, Pos): m/z 460.1 (M+H).

### Example 31

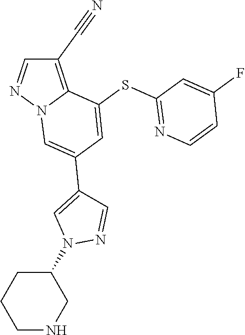 [SMILES: N#Cc1cnn2cc(-c3cnn([C@H]4CCCNC4)c3)cc(Sc3cc(F)ccn3)c12]

### 4-[(4-Fluoro-2-pyridyl) sulfanyl]-6-[1-[(3S)-3-piperidyl]pyrazol-4-yl]pyrazolo[1,5-a]pyridine-3-carbonitrile (31)

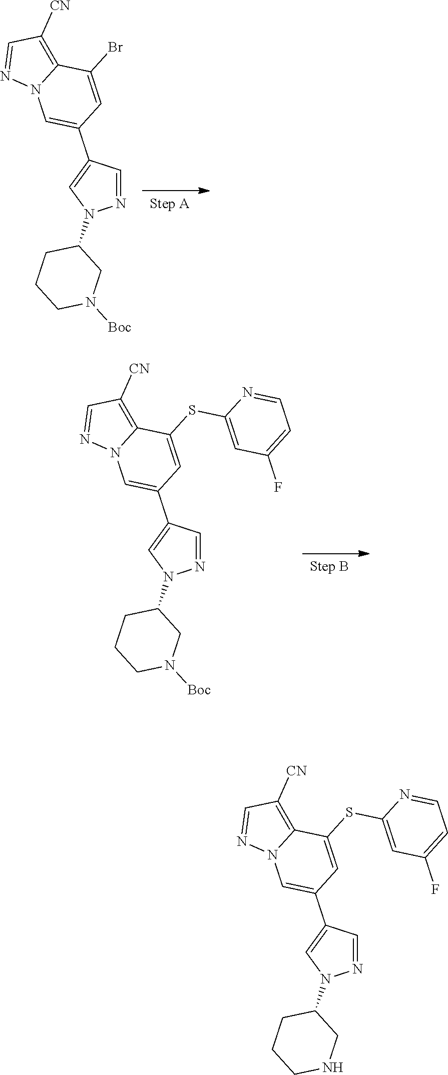

Step A. t-Butyl (3S)-3-[4-[3-cyano-4-[(4-fluoro-2-pyridyl)sulfanyl]pyrazolo[1,5-a]pyridin-6-yl]pyrazol-1-yl]piperidine-1-carboxylate. Synthesized according to Example 25, Step A, substituting 4-fluoropyridine-2-thiol for 5-methylpyridine-2-thiol to give tert-butyl (3S)-3-[4-[3-cyano-4-[(4-fluoro-2-pyridyl)sulfanyl]pyrazolo[1,5-a]pyridin-6-yl]pyrazol-1-yl]piperidine-1-carboxylate (25 mg, 12% yield) as a white solid. LCMS (MM-ES+APCI, Pos): m/z 520.2 (M+H).

Step B. 4-[(4-Fluoro-2-pyridyl) sulfanyl]-6-[1-[(3S)-3-piperidyl]pyrazol-4-yl]pyrazolo[1,5-a]pyridine-3-carbonitrile. Synthesized according to Example 1, Step B, substituting tert-butyl (3S)-3-[4-[3-cyano-4-[(4-fluoro-2-pyridyl)sulfanyl]pyrazolo[1,5-a]pyridin-6-yl]pyrazol-1-yl]piperidine-1-carboxylate for tert-butyl (3S)-3-[4-[3-cyano-4-(2-pyridylsulfanyl)pyrazolo[1,5-a]pyridin-6-yl]pyrazol-1-yl]piperidine-1-carboxylate to give 4-[(4-fluoro-2-pyridyl) sulfanyl]-6-[1-[(3S)-3-piperidyl]pyrazol-4-yl]pyrazolo[1,5-a]pyridine-3-carbonitrile (5 mg, 37% yield) as a white solid. LCMS (MM-ES+APCI, Pos): m/z 420.0 (M+H).

### Example 32

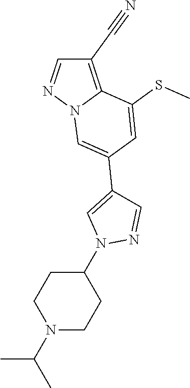 [SMILES: CSc1cc(-c2cnn(C3CCN(C(C)C)CC3)c2)cn2ncc(C#N)c12]

### 6-[1-(1-Isopropyl-4-piperidyl)pyrazol-4-yl]-4-methylsulfanyl-pyrazolo[1,5-a]pyridine-3-carbonitrile (32)

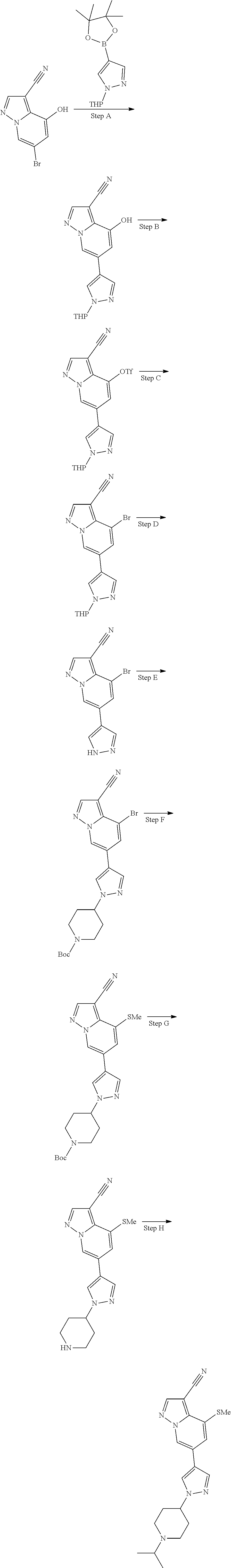

Step A. 4-Hydroxy-6-(1-tetrahydropyran-2-ylpyrazol-4-yl)pyrazolo[1,5-a]pyridine-3-carbonitrile. To a 40 mL flask equipped with a magnetic stir bar were added 6-bromo-4-hydroxy-pyrazolo[1,5-a]pyridine-3-carbonitrile (1.0 g, 4.2 mmol) and 1-tetrahydropyran-2-yl-4-(4,4,5,5-tetramethyl-1,3,2-dioxaborolan-2-yl)pyrazole (1.4 g, 5.0 mmol) followed by the addition of dioxane (20 mL) and H₂O (4 mL). K₂CO₃ (1.2 g, 8.4 mmol), Pd₂(dba)₃ (80 mg, 0.087 mmol) and XPhos (0.10 g, 0.21 mmol) were added into the mixture at 25° C. The flask was purged with nitrogen and heated to 90° C. and stirred for 16 h. Water (40 mL) was added to the reaction and the mixture was transferred to a separatory funnel, the aqueous layer was extracted with ethyl acetate (40 mL×3), combined organic layers were washed with brine (40 mL×2), dried over anhydrous sodium sulfate, filtered, concentrated under reduced pressure and purified by slurry (50% petroleum ether/ethyl acetate) (20 mL). After filtration and drying under vacuum, 4-hydroxy-6-(1-tetrahydropyran-2-ylpyrazol-4-yl)pyrazolo[1,5-a]pyridine-3-carbonitrile (1.3 g, 88% yield,) was obtained as a brown solid. LCMS (MM-ES+APCI, Pos): m/z 226.0 (M-THP+H).

Step B. [3-Cyano-6-(1-tetrahydropyran-2-ylpyrazol-4-yl)pyrazolo[1,5-a]pyridin-4-yl] trifluoromethanesulfonate. Synthesized according to Intermediate 2, Step B substituting 4-hydroxy-6-(1-tetrahydropyran-2-ylpyrazol-4-yl)pyrazolo[1,5-a]pyridine-3-carbonitrile for tert-butyl (3S)-3-[4-(3-cyano-4-hydroxy-pyrazolo[1,5-a]pyridin-6-yl)pyrazol-1-yl]piperidine-1-carboxylate to give [3-cyano-6-(1-tetrahydropyran-2-ylpyrazol-4-yl)pyrazolo[1,5-a]pyridin-4-yl] trifluoromethanesulfonate (1.8 g, 68% yield) as a gray solid. LCMS (MM-ES+APCI, Pos): m/z 464.1 (M+Na+H).

Step C. 4-Bromo-6-(1-tetrahydropyran-2-ylpyrazol-4-yl)pyrazolo[1,5-a]pyridine-3-carbonitrile. Synthesized according to Intermediate 2, Step B substituting [3-cyano-6-(1-tetrahydropyran-2-ylpyrazol-4-yl)pyrazolo[1,5-a]pyridin-4-yl] trifluoromethanesulfonate for tert-butyl (3S)-3-[4-[3-cyano-4-(trifluoromethylsulfonyloxy)pyrazolo[1,5-a]pyridin-6-yl]pyrazol-1-yl]piperidine-1-carboxylate to give 4-bromo-6-(1-tetrahydropyran-2-ylpyrazol-4-yl)pyrazolo[1,5-a]pyridine-3-carbonitrile (680 mg, 43% yield) as a green solid. LCMS (MM-ES+APCI, Pos): m/z 290.0 (M-THP+H).

Step D. 4-Bromo-6-(1H-pyrazol-4-yl)pyrazolo[1,5-a]pyridine-3-carbonitrile. To a 50 mL flask equipped with a magnetic stir bar was added 4-bromo-6-(1-tetrahydropyran-2-ylpyrazol-4-yl)pyrazolo[1,5-a]pyridine-3-carbonitrile (0.68 g, 1.7 mmol) followed by the addition of DCM (5 mL). HCl/dioxane (4 M, 5 mL) was added into the mixture at 25° C. and stirred for 1 h. The mixture was concentrated under reduced pressure to give 4-bromo-6-(1H-pyrazol-4-yl)pyrazolo[1,5-a]pyridine-3-carbonitrile (0.55 g, 98% yield, HCl salt) as a white solid which was used in the next step without further purification.

Step E. t-Butyl 4-[4-(4-bromo-3-cyano-pyrazolo[1,5-a]pyridin-6-yl)pyrazol-1-yl]piperidine-1-carboxylate. To a 40 mL vial equipped with a magnetic stir bar were added 4-bromo-6-(1H-pyrazol-4-yl)pyrazolo[1,5-a]pyridine-3-carbonitrile (0.55 g, 1.7 mmol) and tert-butyl 4-bromopiperidine-1-carboxylate (1.0 g, 3.8 mmol) followed by the addition of DMF (8 mL). Cs₂CO₃ (1.7 g, 5.1 mmol) was added into the mixture at 25° C. The mixture was stirred at 100° ° C. for 48 h. Water (8 mL) was added to the reaction and transferred to a separatory funnel. The aqueous layer was extracted with ethyl acetate (10 mL×3). The combined organic layers were washed with brine (10 mL×2), dried over anhydrous sodium sulfate, filtered, concentrated under reduced pressure and purified by silica gel chromatography (30-50% petroleum ether/ethyl acetate) to give tert-butyl 4-[4-(4-bromo-3-cyano-pyrazolo[1,5-a]pyridin-6-yl)pyrazol-1-yl]piperidine-1-carboxylate (0.41 g, 46% yield) as a white solid. LCMS (MM-ES+APCI, Pos): m/z 416.8 (M-tBu+H).

Step F. Tert-butyl 4-[4-(3-cyano-4-methylsulfanyl-pyrazolo[1,5-a]pyridin-6-yl)pyrazol-1-yl]piperidine-1-carboxylate. To an 8 mL vial equipped with a magnetic stir bar were added tert-butyl 4-[4-(4-bromo-3-cyano-pyrazolo[1,5-a]pyridin-6-yl)pyrazol-1-yl]piperidine-1-carboxylate (0.20 g, 0.38 mmol) and sodium thiomethoxide (41 mg, 0.58 mmol) followed by the addition of toluene (3 mL). Pd(OAc)₂ (9.0 mg, 0.040 mmol), xantphos (25 mg, 0.043 mmol) and Cs₂CO₃ (0.38 g, 1.17 mmol) were added to the mixture at 25° C. The vial was purged with nitrogen and stirred at 100° C. for 16 h. Water (2 mL) was added to the reaction and the mixture was transferred to a separatory funnel. The aqueous layer was extracted with ethyl acetate (5 mL×3). The combined organic layers were washed with brine (5 mL), dried over anhydrous sodium sulfate, filtered, concentrated under reduced pressure and purified by silica gel chromatography (30-50% EtOAc/petroleum ether) to give tert-butyl 4-[4-(3-cyano-4-methylsulfanyl-pyrazolo[1,5-a]pyridin-6-yl)pyrazol-1-yl]piperidine-1-carboxylate (0.14 g, 65% yield) as a yellow solid. LCMS (MM-ES+APCI, Pos): m/z 383.0 (M-tBu+H).

Step G. 4-Methylsulfanyl-6-[1-(4-piperidyl)pyrazol-4-yl]pyrazolo[1,5-a]pyridine-3-carbonitrile. Synthesized according to Example 1, Step B, substituting tert-butyl 4-[4-(3-cyano-4-methylsulfanyl-pyrazolo[1,5-a]pyridin-6-yl)pyrazol-1-yl]piperidine-1-carboxylate for tert-butyl (3S)-3-[4-[3-cyano-4-(2-pyridylsulfanyl)pyrazolo[1,5-a]pyridin-6-yl]pyrazol-1-yl]piperidine-1-carboxylate to give 4-methylsulfanyl-6-[1-(4-piperidyl)pyrazol-4-ylyrazolo[1,5-a]pyridine-3-carbonitrile (88 mg, 99% yield, HCl salt) as a white solid which was used in the next step without further purification.

Step H. 6-[1-(1-Isopropyl-4-piperidyl)pyrazol-4-yl]-4-methylsulfanyl-pyrazolo[1,5-a]pyridine-3-carbonitrile. To an 8 mL flask equipped with a magnetic stir bar were added 4-methylsulfanyl-6-[1-(4-piperidyl)pyrazol-4-yl]pyrazolo[1,5-a]pyridine-3-carbonitrile (68 mg, 0.18 mmol) and 2-iodopropane (48 mg, 0.28 mmol) followed by the addition of MeCN (1.5 mL). K₂CO₃ (78 mg, 0.56 mmol) was added into the mixture and stirred at 70° ° C. for 4 h. Water (5 mL) was added to the reaction and the mixture was transferred to a separatory funnel. The aqueous layer was extracted with dichloromethane (5 mL×3). The combined organic layers were washed with brine (5 mL), dried over anhydrous sodium sulfate, filtered, concentrated under reduced pressure and purified by preparative HPLC (29%-59% acetonitrile/0.04% aqueous ammonia hydroxide+10 mM NH₄HCO₃). After lyophilization, 6-[1-(1-isopropyl-4-piperidyl)pyrazol-4-yl]-4-methylsulfanyl-pyrazolo[1,5-a]pyridine-3-carbonitrile (69 mg, 77% yield) was obtained as a white solid. LCMS (MM-ES+APCI, Pos): m/z 381.1 (M+H).

### Example 33

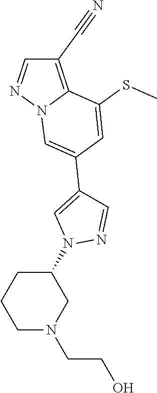 [SMILES: CSc1cc(-c2cnn([C@H]3CCCN(CCO)C3)c2)cn2ncc(C#N)c12]

### 6-[1-[(3S)-1-(2-Hydroxyethyl)-3-piperidyl]pyrazol-4-yl]-4-methylsulfanyl-pyrazolo[1,5-a]pyridine-3-carbonitrile (33)

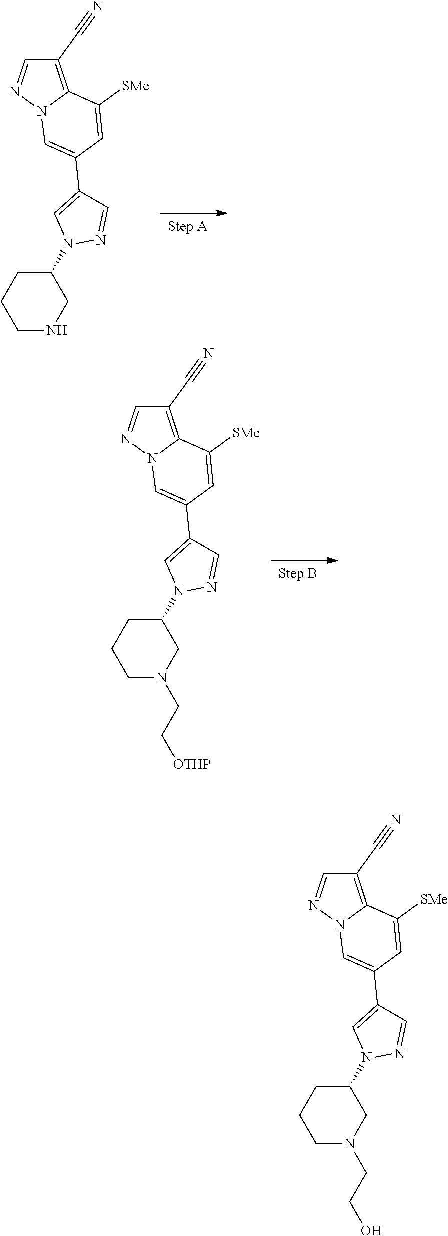

Step A. 4-Methylsulfanyl-6-[1-[(3S)-1-(2-tetrahydropyran-2-yloxyethyl)-3-piperidyl]pyrazol-4-yl]pyrazolo[1,5-a]pyridine-3-carbonitrile. To 8 mL vial were added 2-(2-bromoethoxy)tetrahydropyran (89 mg, 0.42 mmol) and 4-methylsulfanyl-6-[1-[(3S)-3-piperidyl]pyrazol-4-yl]pyrazolo[1,5-a]pyridine-3-carbonitrile (0.12 g, 0.28 mmol) followed by the addition of DMF (1.5 mL). Cs₂CO₃ (0.19 g, 0.59 mmol) was added to the mixture at 25° C. The mixture was stirred at 90° C. for 34 h. Water (2 mL) was added to the reaction and the mixture was transferred to a separatory funnel. The aqueous layer was extracted with ethyl acetate (2 mL×3). The combined organic layers were washed with brine (6 mL), dried over anhydrous sodium sulfate, filtered, and concentrated under reduced pressure to give the crude product 4-methylsulfanyl-6-[1-[(3S)-1-(2-tetrahydropyran-2-yloxyethyl)-3-piperidyl]pyrazol-4-yl]pyrazolo[1,5-a]pyridine-3-carbonitrile (0.14 g, 84% yield) as a brown oil, which was used into the next step without further purification.

Step B. 6-[1-[(3S)-1-(2-hydroxyethyl)-3-piperidyl]pyrazol-4-yl]-4-methylsulfanyl-pyrazolo[1,5-a]pyridine-3-carbonitrile. Synthesized according to Example 1, Step B, substituting 4-methylsulfanyl-6-[1-[(3S)-1-(2-tetrahydropyran-2-yloxyethyl)-3-piperidyl]pyrazol-4-yl]pyrazolo[1,5-a]pyridine-3-carbonitrile for tert-butyl (3S)-3-[4-[3-cyano-4-(2-pyridylsulfanyl)pyrazolo[1,5-a]pyridin-6-yl]pyrazol-1-yl]piperidine-1-carboxylate to give 6-[1-[(3S)-1-(2-hydroxyethyl)-3-piperidyl]pyrazol-4-yl]-4-methylsulfanyl-pyrazolo[1,5-a]pyridine-3-carbonitrile (15 mg, 14% yield) as a white solid. LCMS (MM-ES+APCI, Pos): m/z 382.2 (M+H).

### Example 34

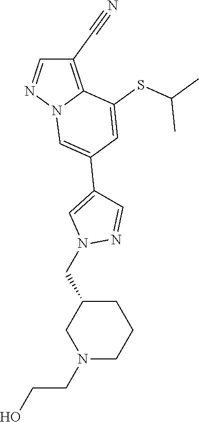 [SMILES: CC(C)Sc1cc(-c2cnn(C[C@@H]3CCCN(CCO)C3)c2)cn2ncc(C#N)c12]

### 6-[1-[[(3R)-1-(2-Hydroxyethyl)-3-piperidyl] methyl] pyrazol-4-yl]-4-isopropylsulfanyl-pyrazolo[1,5-a]pyridine-3-carbonitrile (34)

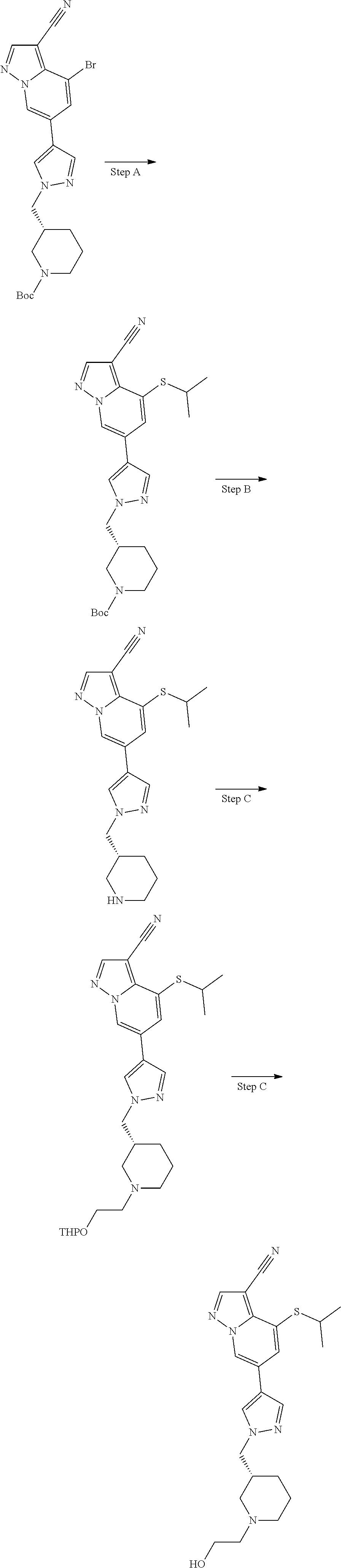

Step A. t-Butyl (3R)-3-[[4-(3-cyano-4-isopropylsulfanyl-pyrazolo[1,5-a]pyridin-6-yl)pyrazol-1-yl] methyl] piperidine-1-carboxylate. Synthesized according to Example 32, Step F substituting tert-butyl (3R)-3-[[4-(4-bromo-3-cyano-pyrazolo[1,5-a]pyridin-6-yl) pyrazol-1-yl]methyl]piperidine-1-carboxylate for tert-butyl 4-[4-(4-bromo-3-cyano-pyrazolo[1,5-a]pyridin-6-yl)pyrazol-1-yl]piperidine-1-carboxylate and propane-2-thiol for methylsulfanylsodium to give tert-butyl (3R)-3-[[4-(3-cyano-4-isopropylsulfanyl-pyrazolo [1,5-a]pyridin-6-yl)pyrazol-1-yl]methyl] piperidine-1-carboxylate (0.26 g, 80% yield) as a yellow solid. LCMS (MM-ES+APCI, Pos): m/z 481.2 (M+H).

Step B. 4-Isopropylsulfanyl-6-[1-[[(3R)-3-piperidyl]methyl] pyrazol-4-yl] pyrazolo[1,5-a]pyridine-3-carbonitrile. Synthesized according to Example 1, Step B, substituting tert-butyl (3R)-3-[[4-(3-cyano-4-isopropylsulfanyl-pyrazolo[1,5-a]pyridin-6-yl) pyrazol-1-yl] methyl]piperidine-1-carboxylate for tert-butyl (3S)-3-[4-[3-cyano-4-(2-pyridylsulfanyl)pyrazolo[1,5-a]pyridin-6-yl]pyrazol-1-yl]piperidine-1-carboxylate to give 4-isopropylsulfanyl-6-[1-[[(3R)-3-piperidyl]methyl] pyrazol-4-yl] pyrazolo[1,5-a]pyridine-3-carbonitrile (0.18 g, crude, HCl salt) as a yellow solid without further purification.

Step C. 6-[1-[[(3R)-1-[2-[t-Butyl (dimethyl) silyl] oxyethyl]-3-piperidyl] methyl] pyrazol-4-yl]-4-isopropylsulfanyl-pyrazolo[1,5-a]pyridine-3-carbonitrile. To 8 mL vial equipped with a magnetic stir bar was added 4-isopropylsulfanyl-6-[1-[[(3R)-3-piperidyl]methyl]pyrazol-4-yl]pyrazolo[1,5-a]pyridine-3-carbonitrile (0.18 g, 0.43 mmol) and 2-bromoethoxy-tert-butyl-dimethylsilane (0.12 mg, 0.52 mmol) followed by the addition of DMF (3 mL). K₂CO₃ (0.12 g, 0.87 mmol) and NaI (13 mg, 0.087 mmol) were added into the mixture at 25° C. The mixture was stirred at 80° C. for 21 h. Water (5 mL) was added to the reaction and the mixture was transferred to a separatory funnel. The aqueous layer was extracted with ethyl acetate (5 mL×3). The combined organic layers were washed with brine (5 mL), dried over anhydrous sodium sulfate, filtered, and concentrated under reduced pressure to give 6-[1-[[(3R)-1-[2-[tert-butyl (dimethyl) silyl] oxyethyl]-3-piperidyl] methyl] pyrazol-4-yl]-4-isopropylsulfanyl-pyrazolo[1,5-a]pyridine-3-carbonitrile (0.23 g, crude product) as a brown oil and used without further purification.

Step D. 6-[1-[[(3R)-1-(2-Hydroxyethyl)-3-piperidyl] methyl] pyrazol-4-yl]-4-isopropylsulfanyl-pyrazolo[1,5-a]pyridine-3-carbonitrile. Synthesized according to Example 1, Step B, substituting 6-[1-[[(3R)-1-[2-[tertbutyl(dimethyl)silyl]oxyethyl]-3-piperidyl]methyl]pyrazol-4-yl]-4-isopropylsulfanyl-pyrazolo[1,5-a]pyridine-3-carbonitrile for tert-butyl (3S)-3-[4-[3-cyano-4-(2-pyridylsulfanyl)pyrazolo[1,5-a]pyridin-6-yl]pyrazol-1-yl]piperidine-1-carboxylate to give 6-[1-[[(3R)-1-(2-hydroxyethyl)-3-piperidyl] methyl] pyrazol-4-yl]-4-isopropylsulfanyl-pyrazolo[1,5-a]pyridine-3-carbonitrile (19 mg, 12% yield) as a white solid. LCMS (MM-ES+APCI, Pos): m/z 425.2 (M+H).

### Example 35

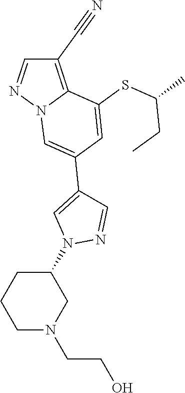 [SMILES: CC[C@@H](C)Sc1cc(-c2cnn([C@H]3CCCN(CCO)C3)c2)cn2ncc(C#N)c12]

### 6-[1-[(3S)-1-(2-Hydroxyethyl)-3-piperidyl]pyrazol-4-yl]-4-[(1R)-1-methylpropyl]sulfanyl-pyrazolo[1,5-a]pyridine-3-carbonitrile (35)

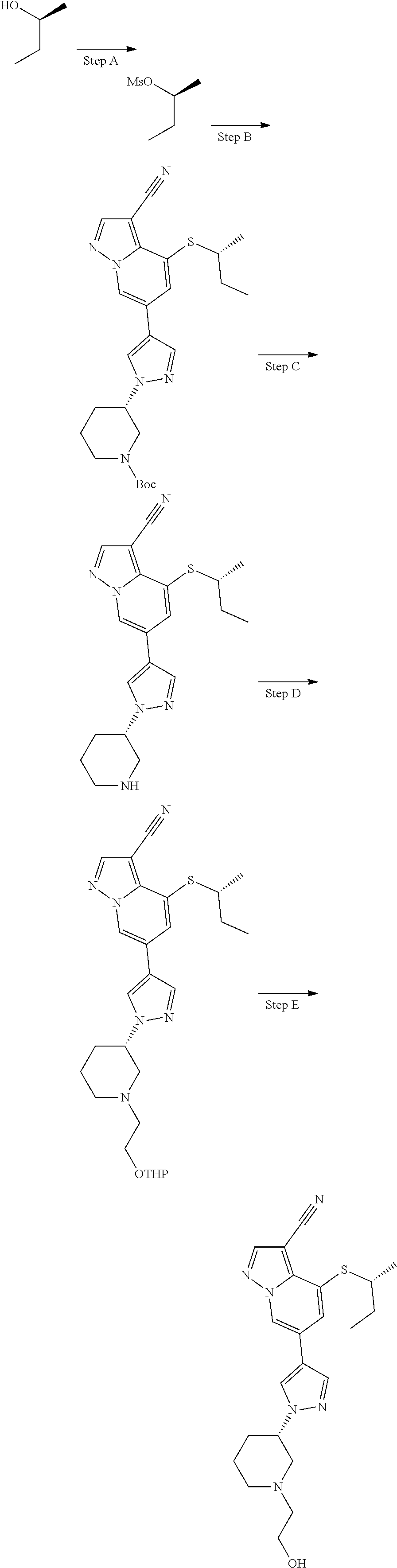

Step A. [(1S)-1-Methylpropyl] methanesulfonate. To a 40 mL round-bottom flask equipped with a magnetic stir bar was added (2S)-butan-2-ol (0.40 g, 5.4 mmol) followed by the addition of DCM (8 mL). Triethylamine (2.3 mL, 16 mmol) and methyl sulfonyl chloride (1.9 g, 11 mmol) were added to the mixture at 0° C. The mixture was stirred at 25° C. for 1 h. Water (10 mL) was added to the reaction and the mixture was transferred to a separatory funnel. The aqueous layer was extracted with ethyl acetate (10 mL×3). The combined organic layers were washed with brine (40 mL), dried over anhydrous sodium sulfate, filtered, and concentrated under reduced pressure to give [(1S)-1-methylpropyl] methane sulfonate (0.80 g, 97% yield) as a yellow liquid which was used into the next step without further purification.

Step B. t-Butyl (3S)-3-[4-[3-cyano-4-[(1R)-1-methylpropyl]sulfanyl-pyrazolo[1,5-a]pyridin-6-yl]pyrazol-1-yl]piperidine-1-carboxylate. To a 40 mL round-bottom flask equipped with a magnetic stir bar was added tert-butyl (3S)-3-[4-(3-cyano-4-sulfanylpyrazolo[1,5-a]pyridin-6-yl)pyrazol-1-yl]piperidine-1-carboxylate (0.35 g, 0.82 mmol) followed by the addition of DMF (5 mL). [(1S)-1-methylpropyl] methanesulfonate (0.19 g, 1.2 mmol) and Cs₂CO₃ (0.40 mg, 1.2 mmol) were added to the mixture at 25° C. The mixture was stirred at 90° C. for 1 h. Water (10 mL) was added to the reaction and the mixture was transferred to a separatory funnel. The aqueous layer was extracted with ethyl acetate (10 mL×3), combined organic layers were washed with brine (35 mL), dried over anhydrous sodium sulfate, filtered, concentrated under reduced pressure and purified by silica gel chromatography (25-50% EtOAc/petroleum ether) to give tert-butyl (3S)-3-[4-[3-cyano-4-[(1R)-1-methylpropyl]sulfanyl-pyrazolo[1,5-a]pyridin-6-yl]pyrazol-1-yl]piperidine-1-carboxylate (0.30 g, 71% yield) as a yellow gum. LCMS (MM-ES+APCI, Pos): m/z 481.0 (M+H).

Step C. 4-[(1R)-1-Methylpropyl] sulfanyl-6-[1-[(3S)-3-piperidyl]pyrazol-4-yl]pyrazolo[1,5-a]pyridine-3-carbonitrile. Synthesized according to Example 1, Step B, substituting tert-butyl (3S)-3-[4-[3-cyano-4-[(1R)-1-methylpropyl]sulfanyl-pyrazolo[1,5-a]pyridin-6-yl]pyrazol-1-yl]piperidine-1-carboxylate for tert-butyl (3S)-3-[4-[3-cyano-4-(2-pyridylsulfanyl)pyrazolo[1,5-a]pyridin-6-yl]pyrazol-1-yl]piperidine-1-carboxylate to give 4-[(1R)-1-methylpropyl] sulfanyl-6-[1-[(3S)-3-piperidyl]pyrazol-4-yl]pyrazolo[1,5-a]pyridine-3-carbonitrile (0.22 g, 96% yield) as yellow solid which was used into the next step without further purification. LCMS (MM-ES+APCI, Pos): m/z 381.3 (M+H).

Step D. 4-(((R)-sec-Butyl)thio)-6-(1-((3S)-1-(2-((tetrahydro-2H-pyran-2-yl)oxy)ethyl)piperidin-3-yl)-1H-pyrazol-4-yl)pyrazolo[1,5-a]pyridine-3-carbonitrile. Synthesized according to Example 35, Step C substituting 4-[(1R)-1-methylpropyl]sulfanyl-6-[1-[(3S)-3-piperidyl]pyrazol-4-yl]pyrazolo[1,5-a]pyridine-3-carbonitrile for 4-isopropylsulfanyl-6-[1-[[(3R)-3-piperidyl]methyl]pyrazol-4-yl]pyrazolo[1,5-a]pyridine-3-carbonitrile to give 4-(((R)-sec-butyl)thio)-6-(1-((3S)-1-(2-((tetrahydro-2H-pyran-2-yl)oxy)ethyl)piperidin-3-yl)-1H-pyrazol-4-yl)pyrazolo[1,5-a]pyridine-3-carbonitrile (0.10 g, 34% yield) as a yellow oil. LCMS (MM-ES+APCI, Pos): m/z 539.5 (M+H).

Step E. 6-[1-[(3S)-1-(2-Hydroxyethyl)-3-piperidyl]pyrazol-4-yl]-4-[(1R)-1-methylpropyl]sulfanyl-pyrazolo[1,5-a]pyridine-3-carbonitrile. Synthesized according to Example 1, Step B, substituting 6-[1-[(3S)-1-[2-[tertbutyl(dimethyl)silyl]oxyethyl]-3-piperidyl]pyrazol-4-yl]-4-[(1R)-1-methylpropyl]sulfanyl-pyrazolo[1,5-a]pyridine-3-carbonitrile for tert-butyl (3S)-3-[4-[3-cyano-4-(2-pyridylsulfanyl)pyrazolo[1,5-a]pyridin-6-yl]pyrazol-1-yl]piperidine-1-carboxylate to give 6-[1-[(3S)-1-(2-hydroxyethyl)-3-piperidyl]pyrazol-4-yl]-4-[(1R)-1-methylpropyl]sulfanyl-pyrazolo[1,5-a]pyridine-3-carbonitrile (43 mg, 68% yield) as an off-white solid. LCMS (MM-ES+APCI, Pos): m/z 425.3 (M+H).

### Example 36

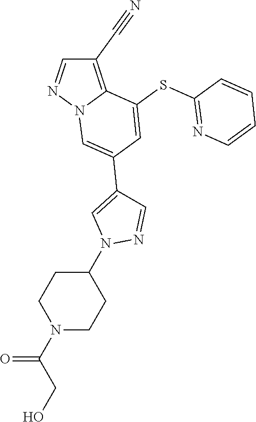 [SMILES: N#Cc1cnn2cc(-c3cnn(C4CCN(C(=O)CO)CC4)c3)cc(Sc3ccccn3)c12]

### 6-[1-[1-(2-Hydroxyacetyl)-4-piperidyl] pyrazol-4-yl]-4-(2-pyridylsulfanyl) pyrazolo[1,5-a]pyridine-3-carbonitrile (36)

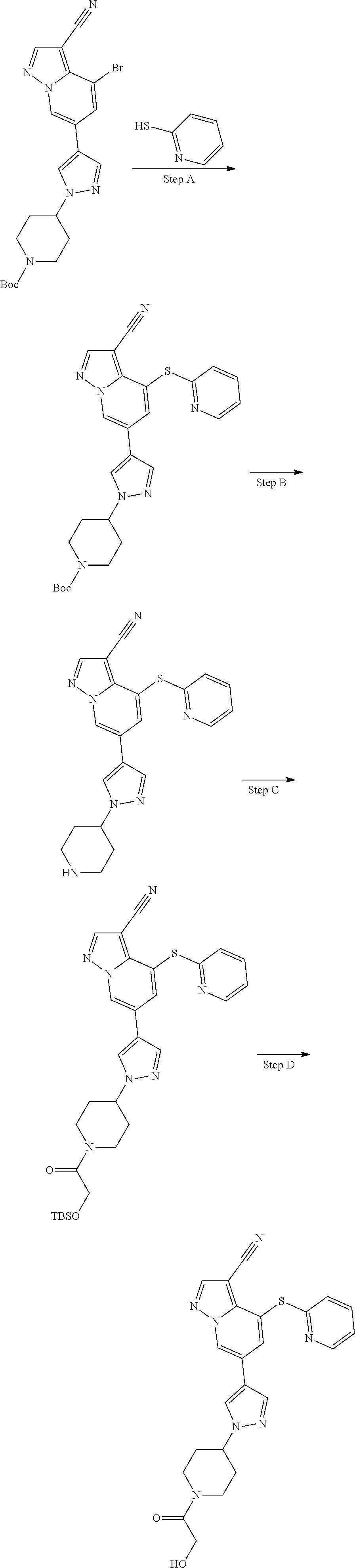 [SMILES: CC(C)(C)OC(=O)N1CCC(n2cc(-c3cc(Br)c4c(C#N)cnn4c3)cn2)CC1 ; Sc1ccccn1 ; CC(C)(C)OC(=O)N1CCC(n2cc(-c3cc(Sc4ccccn4)c4c(C#N)cnn4c3)cn2)CC1 ; N#Cc1cnn2cc(-c3cnn(C4CCNCC4)c3)cc(Sc3ccccn3)c12 ; CC(C)(C)[Si](C)(C)OCC(=O)N1CCC(n2cc(-c3cc(Sc4ccccn4)c4c(C#N)cnn4c3)cn2)CC1 ; N#Cc1cnn2cc(-c3cnn(C4CCN(C(=O)CO)CC4)c3)cc(Sc3ccccn3)c12]

Step A. t-Butyl 4-[4-[3-cyano-4-(2-pyridylsulfanyl)pyrazolo[1,5-a]pyridin-6-yl]pyrazol-1-yl] piperidine-1-carboxylate. Synthesized according to Example 1, Step A, substituting tert-butyl 4-[4-(4-bromo-3-cyano-pyrazolo[1,5-a]pyridin-6-yl)pyrazol-1-yl]piperidine-1-carboxylate for tert-butyl (3S)-3-[4-(4-bromo-3-cyano-pyrazolo[1,5-a]pyridin-6-yl)pyrazol-1-yl]piperidine-1-carboxylate to give tert-butyl 4-[4-[3-cyano-4-(2-pyridylsulfanyl)pyrazolo[1,5-a]pyridin-6-yl]pyrazol-1-yl] piperidine-1-carboxylate (0.96 g, 59% yield) as a light yellow solid.

Step B. 6-[1-(4-Piperidyl)pyrazol-4-yl]-4-(2-pyridylsulfanyl) pyrazolo[1,5-a]pyridine-3-carbonitrile. Synthesized according to Example 1, Step B, substituting tert-butyl 4-[4-[3-cyano-4-(2-pyridylsulfanyl) pyrazolo[1,5-a]pyridin-6-yl]pyrazol-1-yl] piperidine-1-carboxylate for tert-butyl (3S)-3-[4-[3-cyano-4-(2-pyridylsulfanyl)pyrazolo[1,5-a]pyridin-6-yl]pyrazol-1-yl]piperidine-1-carboxylate to give 6-[1-(4-piperidyl)pyrazol-4-yl]-4-(2-pyridylsulfanyl) pyrazolo[1,5-a]pyridine-3-carbonitrile (0.80 g, crude product, HCl salt) as a light yellow solid which was used without further purification.

Step C. 6-[1-[1-[2-[t-Butyl(dimethyl)silyl]oxyacetyl]-4-piperidyl]pyrazol-4-yl]-4-(2-pyridylsulfanyl) pyrazolo[1,5-a]pyridine-3-carbonitrile. To a 40 mL vial equipped with a magnetic stir bar was added 2-[tert-butyl (dimethyl)silyl] oxyacetic acid (98 mg, 0.51 mmol), HOBt (98 mg, 0.72 mmol) and EDCI (0.14 g, 0.70 mmol) followed by the addition of DCM (5 mL). DIEA (0.30 mL, 1.7 mmol) was added to the mixture at 25° C. The mixture was stirred at 25° C. for 30 min. 6-[1-(4-piperidyl)pyrazol-4-yl]-4-(2-pyridylsulfanyl)pyrazolo[1,5-a]pyridine-3-carbonitrile (0.15 g, 0.34 mmol) was added and the mixture stirred at 25° C. for 20.5 h. Water (10 mL) was added to the reaction and the mixture was transferred to a separatory funnel. The aqueous layer was extracted with ethyl acetate (10 mL×3). The combined organic layers were washed with brine (10 mL), dried over anhydrous sodium sulfate, filtered, concentrated under reduced pressure and purified by silica gel column chromatography (50-55% EtOAc/petroleum ether) to give 6-[1-[1-[2-[tert-butyl(dimethyl)silyl]oxyacetyl]-4-piperidyl]pyrazol-4-yl]-4-(2-pyridylsulfanyl) pyrazolo[1,5-a]pyridine-3-carbonitrile (60 mg, 0.097 mmol, 28% yield) as a brown solid. LCMS (MM-ES+APCI, Pos): m/z 574.3 (M+H).

Step D. 6-[1-[1-(2-Hydroxyacetyl)-4-piperidyl] pyrazol-4-yl]-4-(2-pyridylsulfanyl) pyrazolo[1,5-a]pyridine-3-carbonitrile. Synthesized according to Example 1, Step B, substituting 6-[1-[1-[2-[tert-butyl(dimethyl)silyl]oxyacetyl]-4-piperidyl] pyrazol-4-yl]-4-(2-pyridylsulfanyl) pyrazolo[1,5-a]pyridine-3-carbonitrile for tert-butyl (3S)-3-[4-[3-cyano-4-(2-pyridylsulfanyl)pyrazolo[1,5-a]pyridin-6-yl]pyrazol-1-yl]piperidine-1-carboxylate to give 6-[1-[1-(2-hydroxyacetyl)-4-piperidyl] pyrazol-4-yl]-4-(2-pyridylsulfanyl) pyrazolo[1,5-a]pyridine-3-carbonitrile (21 mg, 64% yield) as a white solid. LCMS (MM-ES+APCI, Pos): m/z 460.0 (M+H).

### Example 37

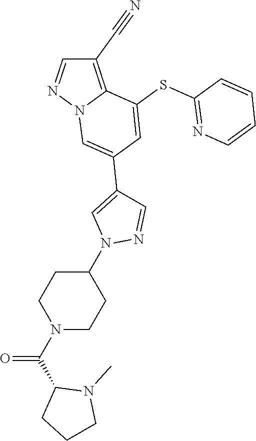 [SMILES: CN1CCC[C@@H]1C(=O)N1CCC(n2cc(-c3cc(Sc4ccccn4)c4c(C#N)cnn4c3)cn2)CC1]

### 6-[1-[1-[(2R)-1-Methylpyrrolidine-2-carbonyl]-4-piperidyl]pyrazol-4-yl]-4-(2-pyridylsulfanyl) pyrazolo[1,5-a] pyridine-3-carbonitrile (37)

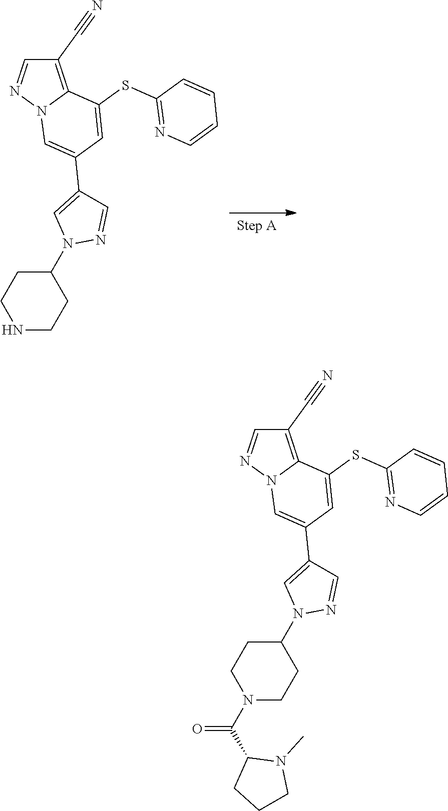 [SMILES: N#Cc1cnn2cc(-c3cnn(C4CCNCC4)c3)cc(Sc3ccccn3)c12 ; CN1CCC[C@@H]1C(=O)N1CCC(n2cc(-c3cc(Sc4ccccn4)c4c(C#N)cnn4c3)cn2)CC1]

Step A. 6-[1-[1-[(2R)-1-Methylpyrrolidine-2-carbonyl]-4-piperidyl]pyrazol-4-yl]-4-(2-pyridylsulfanyl) pyrazolo[1,5-a] pyridine-3-carbonitrile. To an 8 mL vial equipped with a magnetic stir bar was added (2R)-1-methylpyrrolidine-2-carboxylic acid (60 mg, 0.46 mmol) and HATU (0.22 g, 0.57 mmol) followed by the addition of DMF (3 mL). DIEA (0.19 mL, 1.1 mmol) was added into the mixture at 25° C. and stirred for 30 min. 6-[1-(4-piperidyl) pyrazol-4-yl]-4-(2-pyridylsulfanyl) pyrazolo[1,5-a]pyridine-3-carbonitrile (0.12 g, 0.27 mmol) was added into the mixture and stirred at 25° C. for 2 h. Water (10 mL) was added to the reaction and the mixture was transferred to a separatory funnel, the aqueous layer was extracted with ethyl acetate (10 mL×3), combined organic layers were washed with brine (10 mL), dried over anhydrous sodium sulfate, filtered, concentrated under reduced pressure and purified by preparative HPLC: (19%-49% B acetonitrile/water (10 mM NH₄HCO₃). After lyophilization, 6-[1-[1-[(2R)-1-methylpyrrolidine-2-carbonyl]-4-piperidyl]pyrazol-4-yl]-4-(2-pyridylsulfanyl) pyrazolo[1,5-a] pyridine-3-carbonitrile (73 mg, 54% yield) was obtained as a yellow solid. LCMS (MM-ES+APCI, Pos): m/z 513.3 (M+H).

### Example 38

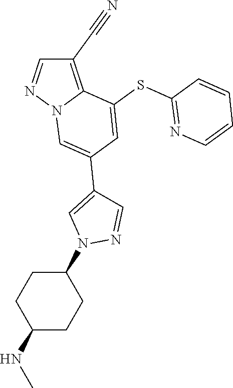 [SMILES: CN[C@H]1CC[C@@H](n2cc(-c3cc(Sc4ccccn4)c4c(C#N)cnn4c3)cn2)CC1]

### 6-[1-[4-(Methylamino)cyclohexyl]pyrazol-4-yl]-4-(2-pyridylsulfanyl)pyrazolo[1,5-a]pyridine-3-carbonitrile (38)

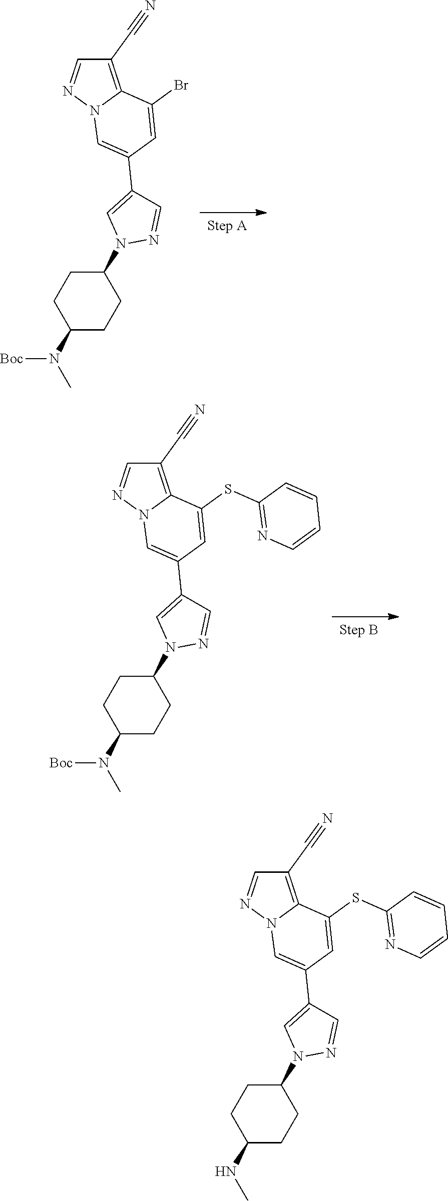 [SMILES: CN(C(=O)OC(C)(C)C)[C@H]1CC[C@@H](n2cc(-c3cc(Br)c4c(C#N)cnn4c3)cn2)CC1 ; CN(C(=O)OC(C)(C)C)[C@H]1CC[C@@H](n2cc(-c3cc(Sc4ccccn4)c4c(C#N)cnn4c3)cn2)CC1 ; CN[C@H]1CC[C@@H](n2cc(-c3cc(Sc4ccccn4)c4c(C#N)cnn4c3)cn2)CC1]

Step A. t-Butyl N-[4-[4-[3-cyano-4-(2-pyridylsulfanyl)pyrazolo[1,5-a]pyridin-6-yl]pyrazol-1-yl]cyclohexyl]-N-methyl-carbamate. Synthesized according to Example 1, Step A, substituting tert-butyl N-[4-[4-(4-bromo-3-cyano-pyrazolo[1,5-a]pyridin-6-yl)pyrazol-1-yl]cyclohexyl]-N-methyl-carbamate for tert-butyl (3S)-3-[4-(4-bromo-3-cyano-pyrazolo[1,5-a]pyridin-6-yl)pyrazol-1-yl]piperidine-1-carboxylate to give tert-butyl N-[4-[4-[3-cyano-4-(2-pyridylsulfanyl)pyrazolo[1,5-a]pyridin-6-yl]pyrazol-1-yl]cyclohexyl]-N-methyl-carbamate (80 mg, 63% yield) as a white solid. LCMS (MM-ES+APCI, Pos): m/z 530.2 (M+H).

Step B. 6-[1-[4-(Methylamino)cyclohexyl]pyrazol-4-yl]-4-(2-pyridylsulfanyl)pyrazolo[1,5-a]pyridine-3-carbonitrile. Synthesized according to Example 1, Step B, substituting tert-butyl N-[4-[4-[3-cyano-4-(2-pyridylsulfanyl)pyrazolo[1,5-a]pyridin-6-yl]pyrazol-1-yl]cyclohexyl]-N-methyl-carbamate for tert-butyl (3S)-3-[4-[3-cyano-4-(2-pyridylsulfanyl)pyrazolo[1,5-a]pyridin-6-yl]pyrazol-1-yl]piperidine-1-carboxylate to give 6-[1-[4-(methylamino)cyclohexyl]pyrazol-4-yl]-4-(2-pyridylsulfanyl)pyrazolo[1,5-a]pyridine-3-carbonitrile (37 mg, 67% yield) as a white solid. LCMS (MM-ES+APCI, Pos): m/z 430.4 (M+H).

### Example 39

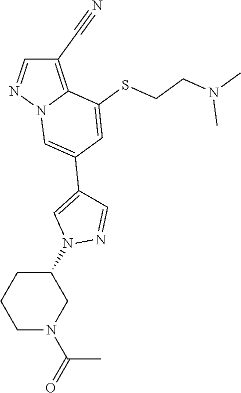 [SMILES: CC(=O)N1CCC[C@H](n2cc(-c3cc(SCCN(C)C)c4c(C#N)cnn4c3)cn2)C1]

### 6-[1-[(3S)-1-Acetyl-3-piperidyl]pyrazol-4-yl]-4-[2-(dimethylamino)ethylsulfanyl]pyrazolo[1,5-a]pyridine-3-carbonitrile (39)

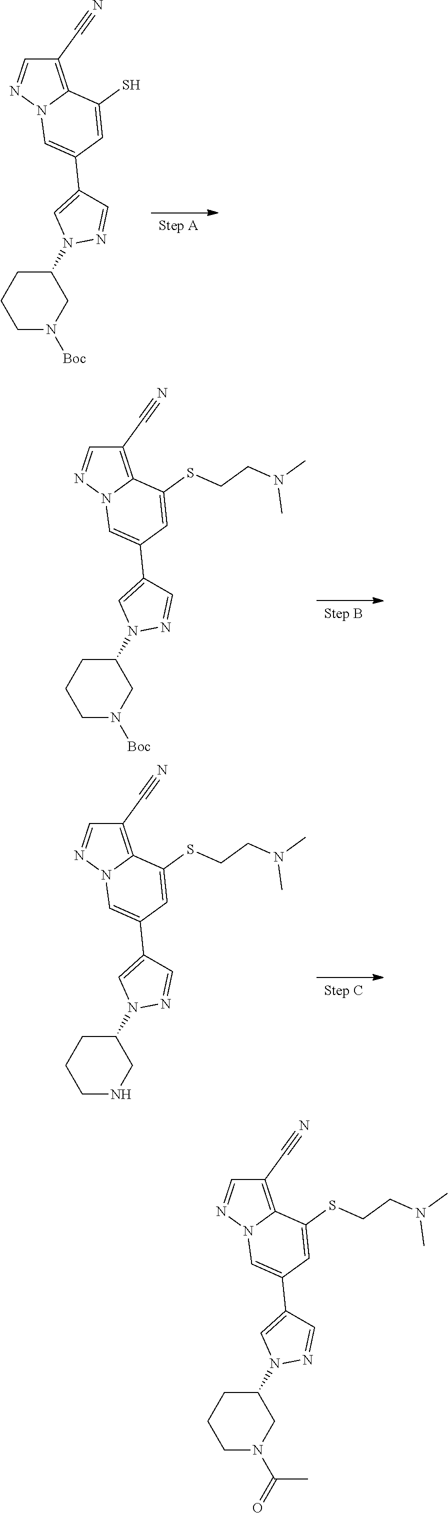

Step A. t-Butyl (3S)-3-[4-[3-cyano-4-[2-(dimethylamino)ethylsulfanyl]pyrazolo[1,5-a]pyridin-6-yl]pyrazol-1-yl]piperidine-1-carboxylate. Synthesized according to Example 10, Step A substituting 2-bromo-N,N-dimethyl-ethanamine for [(1S)-1-(3,5-dichloro-4-pyridyl)ethyl] methane sulfonate to give tert-butyl (3S)-3-[4-[3-cyano-4-[2-(dimethylamino)ethylsulfanyl]pyrazolo[1,5-a]pyridin-6-yl]pyrazol-1-yl]piperidine-1-carboxylate (0.17 g, 96% yield) as a yellow oil. LCMS (MM-ES+APCI, Pos): m/z 496.5 (M+H).

Step B. 4-[2-(Dimethylamino)ethylsulfanyl]-6-[1-[(3S)-3-piperidyl]pyrazol-4-yl]pyrazolo[1,5-a]pyridine-3-carbonitrile. Synthesized according to Example 1, Step B, substituting tert-butyl (3S)-3-[4-[3-cyano-4-[2-(dimethylamino)ethylsulfanyl]pyrazolo[1,5-a]pyridin-6-yl]pyrazol-1-yl]piperidine-1-carboxylate for tert-butyl (3S)-3-[4-[3-cyano-4-(2-pyridylsulfanyl)pyrazolo[1,5-a]pyridin-6-yl]pyrazol-1-yl]piperidine-1-carboxylate to give 4-[2-(dimethylamino)ethylsulfanyl]-6-[1-[(3S)-3-piperidyl]pyrazol-4-yl]pyrazolo[1,5-a]pyridine-3-carbonitrile (0.12 g, 99% yield) as a yellow solid.

Step C. 6-[1-[(3S)-1-Acetyl-3-piperidyl]pyrazol-4-yl]-4-[2-(dimethylamino)ethylsulfanyl]pyrazolo[1,5-a]pyridine-3-carbonitrile. To a 8 mL vial equipped with a magnetic stir bar was added 4-[2-(dimethylamino)ethylsulfanyl]-6-[1-[(3S)-3-piperidyl]pyrazol-4-yl]pyrazolo[1,5-a]pyridine-3-carbonitrile (0.10 g, 0.23 mmol) followed by the addition of DCM (2 mL). The solution was cooled to 0° C., triethylamine (70 mg, 0.69 mmol) and acetyl chloride (22 mg, 0.28 mmol) were added dropwise at 0° C. The mixture was allowed to warm to 25° C. and stirred for 0.5 h. The mixture was concentrated under reduced pressure and purified by preparative HPLC: (19%-49% acetonitrile/0.05% aqueous ammonia hydroxide). After lyophilization, 6-[1-[(3S)-1-acetyl-3-piperidyl]pyrazol-4-yl]-4-[2-(dimethylamino)ethylsulfanyl]pyrazolo[1,5-a]pyridine-3-carbonitrile (46 mg, 45% yield) was obtained as a yellow solid. LCMS (MM-ES+APCI, Pos): m/z 438.2 (M+H).

### Example 40

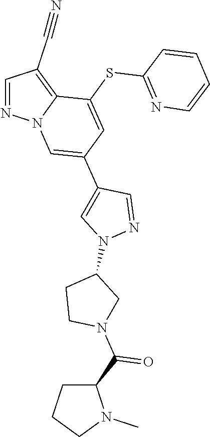 [SMILES: CN1CCC[C@H]1C(=O)N1CC[C@H](n2cc(-c3cc(Sc4ccccn4)c4c(C#N)cnn4c3)cn2)C1]

### 6-(1-((S)-1-(Methyl-L-prolyl)pyrrolidin-3-yl)-1H-pyrazol-4-yl)-4-(pyridin-2-ylthio)pyrazolo[1,5-a]pyridine-3-carbonitrile (40)

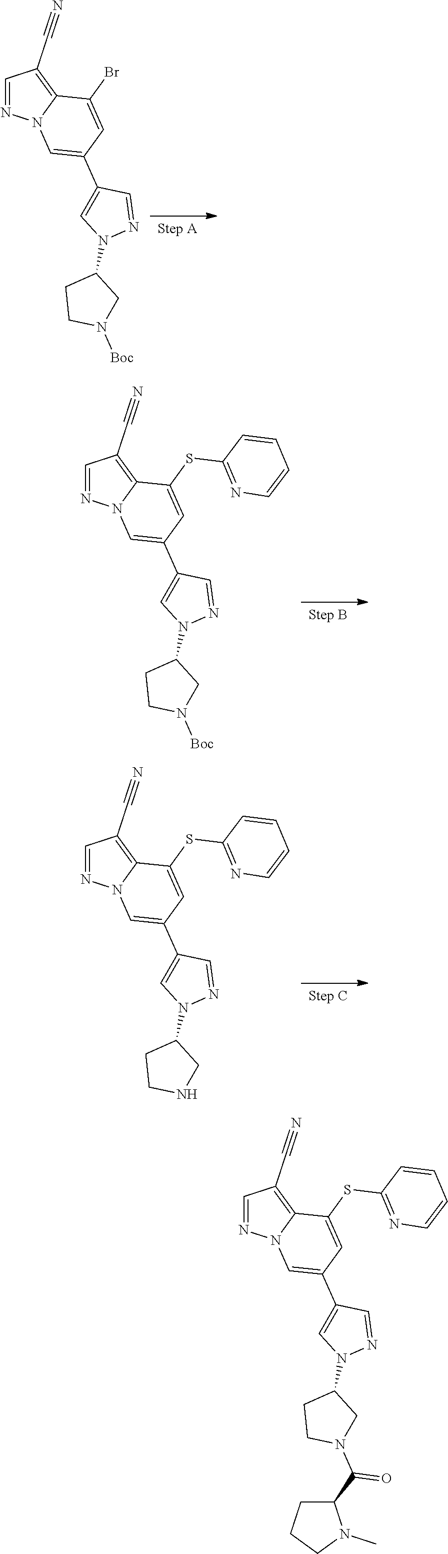

Step A. t-Butyl (S)-3-(4-(3-cyano-4-(pyridin-2-ylthio)pyrazolo[1,5-a]pyridin-6-yl)-1H-pyrazol-1-yl)pyrrolidine-1-carboxylate. Synthesized according to Experiment 1, Step A, substituting tert-butyl (S)-3-(4-(4-bromo-3-cyanopyrazolo[1,5-a]pyridin-6-yl)-1H-pyrazol-1-yl)pyrrolidine-1-carboxylate for tert-butyl (3S)-3-[4-(4-bromo-3-cyano-pyrazolo[1,5-a]pyridin-6-yl)pyrazol-1-yl]piperidine-1-carboxylate to give tert-butyl (S)-3-(4-(3-cyano-4-(pyridin-2-ylthio)pyrazolo[1,5-a]pyridin-6-yl)-1H-pyrazol-1-yl)pyrrolidine-1-carboxylate (9 mg, 53% yield). LCMS (MM-ES+APCI, Pos): m/z 488.3 (M+H).

Step B. (S)-4-(Pyridin-2-ylthio)-6-(1-(pyrrolidin-3-yl)-1H-pyrazol-4-yl)pyrazolo[1,5-a]pyridine-3-carbonitrile. Synthesized according to Example 1, Step B, substituting tert-butyl (S)-3-(4-(3-cyano-4-(pyridin-2-ylthio)pyrazolo[1,5-a]pyridin-6-yl)-1H-pyrazol-1-yl)pyrrolidine-1-carboxylate for tert-butyl (3S)-3-[4-[3-cyano-4-(2-pyridylsulfanyl)pyrazolo[1,5-a]pyridin-6-yl]pyrazol-1-yl]piperidine-1-carboxylate to give (S)-4-(pyridin-2-ylthio)-6-(1-(pyrrolidin-3-yl)-1H-pyrazol-4-yl)pyrazolo[1,5-a]pyridine-3-carbonitrile (84 mg, quant yield). LCMS (MM-ES+APCI, Pos): m/z 388.2 (M+H).

Step C. 6-(1-((S)-1-(Methyl-L-prolyl)pyrrolidin-3-yl)-1H-pyrazol-4-yl)-4-(pyridin-2-ylthio)pyrazolo[1,5-a]pyridine-3-carbonitrile. Synthesized according to Example 37, Step A substituting (S)-4-(pyridin-2-ylthio)-6-(1-(pyrrolidin-3-yl)-1H-pyrazol-4-yl)pyrazolo[1,5-a]pyridine-3-carbonitrile for 6-[1-(4-piperidyl) pyrazol-4-yl]-4-(2-pyridylsulfanyl) pyrazolo[1,5-a]pyridine-3-carbonitrile and (2S)-1-methylpyrrolidine-2-carboxylic acid for (2R)-1-methylpyrrolidine-2-carboxylic acid to give 6-(1-((S)-1-(methyl-L-prolyl)pyrrolidin-3-yl)-1H-pyrazol-4-yl)-4-(pyridin-2-ylthio)pyrazolo[1,5-a]pyridine-3-carbonitrile (20 mg, 65% yield). LCMS (MM-ES+APCI, Pos): m/z 499.3 (M+H).

### Example 41

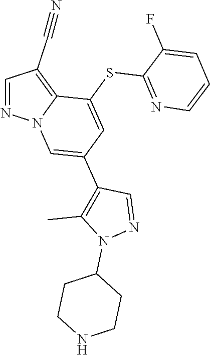 [SMILES: Cc1c(-c2cc(Sc3ncccc3F)c3c(C#N)cnn3c2)cnn1C1CCNCC1]

### 4-[(3-Fluoro-2-pyridyl) sulfanyl]-6-[5-methyl-1-(4-piperidyl) pyrazol-4-yl] pyrazolo[1,5-a]pyridine-3-carbonitrile (41)

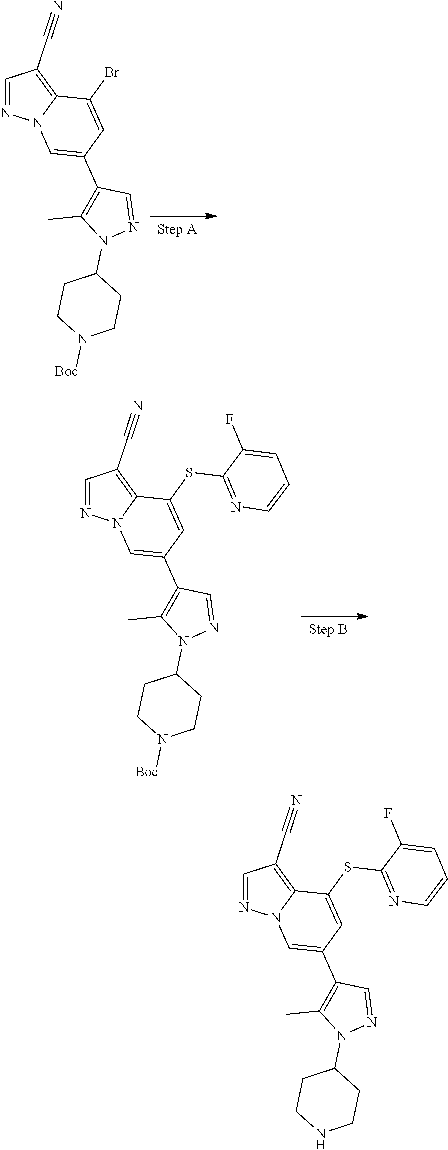 [SMILES: Cc1c(-c2cc(Br)c3c(C#N)cnn3c2)cnn1C1CCN(C(=O)OC(C)(C)C)CC1 ; Cc1c(-c2cc(Sc3ncccc3F)c3c(C#N)cnn3c2)cnn1C1CCN(C(=O)OC(C)(C)C)CC1 ; Cc1c(-c2cc(Sc3ncccc3F)c3c(C#N)cnn3c2)cnn1C1CCNCC1]

Step A. t-Butyl 4-[4-[3-cyano-4-[(3-fluoro-2-pyridyl) sulfanyl] pyrazolo[1,5-a]pyridin-6-yl]-5-methyl-pyrazol-1-yl]piperidine-1-carboxylate. Synthesized according to Experiment 1, Step A, substituting tert-butyl 4-[4-(4-bromo-3-cyano-pyrazolo[1,5-a]pyridin-6-yl)-5-methyl-pyrazol-1-yl]piperidine-1-carboxylate for tert-butyl (3S)-3-[4-(4-bromo-3-cyano-pyrazolo[1,5-a]pyridin-6-yl)pyrazol-1-yl]piperidine-1-carboxylate to give tert-butyl 4-[4-[3-cyano-4-[(3-fluoro-2-pyridyl) sulfanyl] pyrazolo[1,5-a]pyridin-6-yl]-5-methyl-pyrazol-1-yl]piperidine-1-carboxylate (2.8 g, 99% yield) as a yellow solid. LCMS (MM-ES+APCI, Pos): m/z 534.2 (M+H).

Step B. 4-[(3-Fluoro-2-pyridyl) sulfanyl]-6-[5-methyl-1-(4-piperidyl) pyrazol-4-yl]pyrazolo[1,5-a]pyridine-3-carbonitrile. Synthesized according to Example 1, Step B, substituting tert-butyl 4-[4-[3-cyano-4-[(3-fluoro-2-pyridyl) sulfanyl] pyrazolo[1,5-a]pyridin-6-yl]-5-methyl-pyrazol-1-yl]piperidine-1-carboxylate for tert-butyl (3S)-3-[4-[3-cyano-4-(2-pyridylsulfanyl)pyrazolo[1,5-a]pyridin-6-yl]pyrazol-1-yl]piperidine-1-carboxylate to give 4-[(3-fluoro-2-pyridyl) sulfanyl]-6-[5-methyl-1-(4-piperidyl) pyrazol-4-yl] pyrazolo[1,5-a]pyridine-3-carbonitrile (35 mg, 46% yield) as an off-white solid. LCMS (MM-ES+APCI, Pos): m/z 434.1 (M+H).

### Example 42

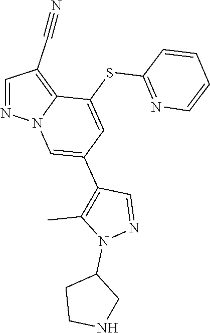 [SMILES: Cc1c(-c2cc(Sc3ccccn3)c3c(C#N)cnn3c2)cnn1C1CCNC1]

### 4-[(3-Fluoro-2-pyridyl)sulfanyl]-6-[5-methyl-1-[(3R)-pyrrolidin-3-yl]pyrazol-4-yl]pyrazolo[1,5-a]pyridine-3-carbonitrile (42)

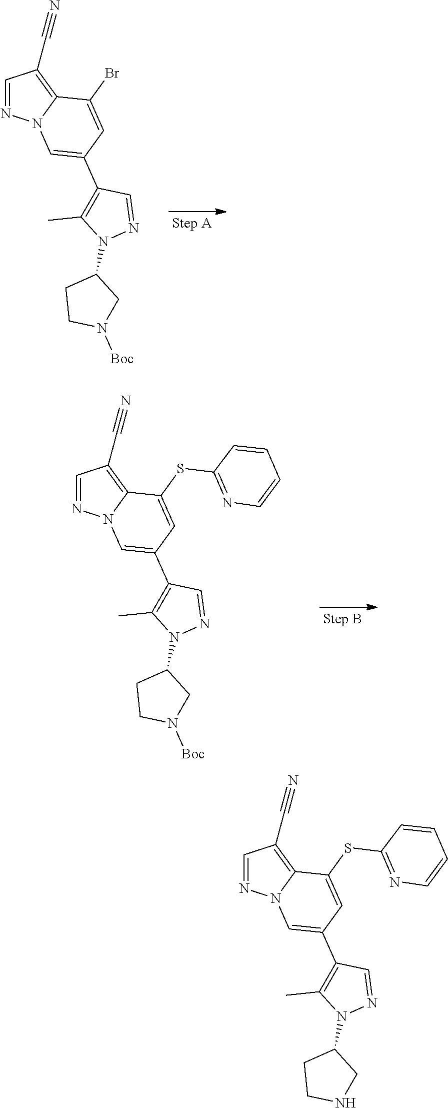

Step A. t-Butyl (3R)-3-[4-[3-cyano-4-[(3-fluoro-2-pyridyl)sulfanyl]pyrazolo[1,5-a]pyridin-6-yl]-5-methyl-pyrazol-1-yl]pyrrolidine-1-carboxylate. Synthesized according to Experiment 1, Step A, substituting tert-butyl (3R)-3-[4-(4-bromo-3-cyano-pyrazolo[1,5-a]pyridin-6-yl)-5-methyl-pyrazol-1-yl]pyrrolidine-1-carboxylate for tert-butyl (3S)-3-[4-(4-bromo-3-cyano-pyrazolo[1,5-a]pyridin-6-yl)pyrazol-1-yl]piperidine-1-carboxylate to give tert-butyl (3R)-3-[4-[3-cyano-4-[(3-fluoro-2-pyridyl)sulfanyl]pyrazolo[1,5-a]pyridin-6-yl]-5-methyl-pyrazol-1-yl]pyrrolidine-1-carboxylate (88 mg, 66% yield) as a yellow oil. LCMS (MM-ES+APCI, Pos): m/z 520.1 (M+H).

Step B. 4-[(3-Fluoro-2-pyridyl)sulfanyl]-6-[5-methyl-1-[(3R)-pyrrolidin-3-yl]pyrazol-4-yl]pyrazolo[1,5-a]pyridine-3-carbonitrile. Synthesized according to Example 1, Step B, substituting tert-butyl (3R)-3-[4-[3-cyano-4-[(3-fluoro-2-pyridyl)sulfanyl]pyrazolo[1,5-a]pyridin-6-yl]-5-methyl-pyrazol-1-yl]pyrrolidine-1-carboxylate for tert-butyl (3S)-3-[4-[3-cyano-4-(2-pyridylsulfanyl)pyrazolo[1,5-a]pyridin-6-yl]pyrazol-1-yl]piperidine-1-carboxylate to give 4-[(3-fluoro-2-pyridyl)sulfanyl]-6-[5-methyl-1-[(3R)-pyrrolidin-3-yl]pyrazol-4-yl]pyrazolo[1,5-a]pyridine-3-carbonitrile (34 mg, 49% yield) as a white solid. LCMS (MM-ES+APCI, Pos): m/z 420.0 (M+H).

### Example 43

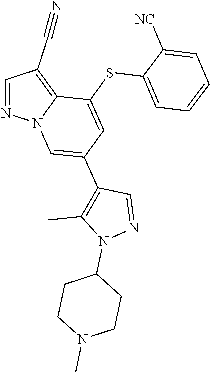 [SMILES: Cc1c(-c2cc(Sc3ccccc3C#N)c3c(C#N)cnn3c2)cnn1C1CCN(C)CC1]

### 4-(2-Cyanophenyl)sulfanyl-6-[5-methyl-1-(1-methyl-4-piperidyl)pyrazol-4-yl]pyrazolo[1,5-a]pyridine-3-cabibutrile (43)

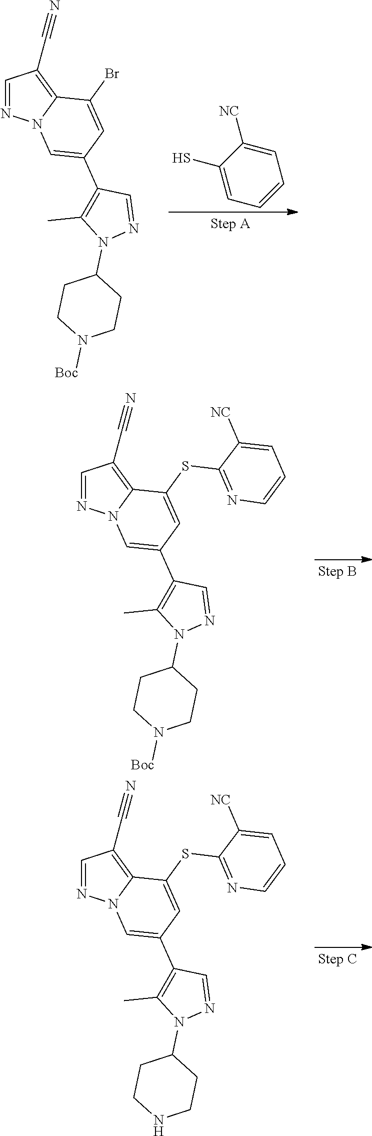

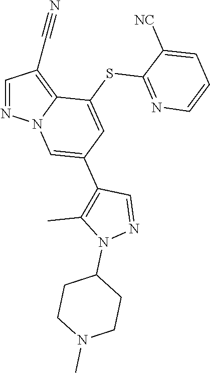 [SMILES: Cc1c(-c2cc(Sc3ncccc3C#N)c3c(C#N)cnn3c2)cnn1C1CCN(C)CC1]

Step A. t-Butyl 4-[4-[3-cyano-4-(2-cyanophenyl)sulfanyl-pyrazolo[1,5-a]pyridin-6-yl]-5-methyl-pyrazol-1-yl]piperidine-1-carboxylate. Synthesized according to Experiment 1, Step A, substituting tert-butyl 4-[4-(4-bromo-3-cyano-pyrazolo[1,5-a]pyridin-6-yl)-5-methyl-pyrazol-1-yl]piperidine-1-carboxylate for tert-butyl (3S)-3-[4-(4-bromo-3-cyano-pyrazolo[1,5-a]pyridin-6-yl)pyrazol-1-yl]piperidine-1-carboxylate and 2-sulfanylbenzonitrile for pyridine-2-thiol to give tert-butyl 4-[4-[3-cyano-4-(2-cyanophenyl)sulfanyl-pyrazolo[1,5-a]pyridin-6-yl]-5-methyl-pyrazol-1-yl]piperidine-1-carboxylate (166 mg, 74% yield) as a yellow oil. LCMS (MM-ES+APCI, Pos): m/z 540.3 (M+H).

Step B. 4-(2-Cyanophenyl)sulfanyl-6-[5-methyl-1-(4-piperidyl)pyrazol-4-yl]pyrazolo[1,5-a]pyridine-3-carbonitrile. Synthesized according to Example 1, Step B, substituting tert-butyl 4-[4-[3-cyano-4-(2-cyanophenyl)sulfanyl-pyrazolo[1,5-a]pyridin-6-yl]-5-methyl-pyrazol-1-yl]piperidine-1-carboxylate for tert-butyl (3S)-3-[4-[3-cyano-4-(2-pyridylsulfanyl)pyrazolo[1,5-a]pyridin-6-yl]pyrazol-1-yl]piperidine-1-carboxylate to give 4-(2-cyanophenyl)sulfanyl-6-[5-methyl-1-(4-piperidyl)pyrazol-4-yl]pyrazolo[1,5-a]pyridine-3-carbonitrile (24 mg, 48% yield) as a white solid.

Step C. 4-(2-Cyanophenyl)sulfanyl-6-[5-methyl-1-(1-methyl-4-piperidyl)pyrazol-4-yl]pyrazolo[1,5-a]pyridine-3-carbonitrile. To a 40 mL vial equipped with a magnetic stir bar was added 4-(2-cyanophenyl)sulfanyl-6-[5-methyl-1-(4-piperidyl)pyrazol-4-yl]pyrazolo[1,5-a]pyridine-3-carbonitrile (70 mg, 0.15 mmol, HCl salt) followed by the addition of DCE (10 mL). Acetic acid (0.05 mL, 0.88 mmol) and formaldehyde (0.021 mL, 0.29 mmol) were added to the mixture and the reaction stirred at 30° C. for 0.5 hr. NaBH(OAc)₃ (94 mg, 0.44 mmol) was added under nitrogen. The reaction was stirred at 30° C. for 1 h, concentrated under reduced pressure and purified by preparative HPLC: (25%-55% acetonitrile/0.04% aqueous ammonia hydroxide+10 mM NH₄HCO₃) to give 4-(2-cyanophenyl)sulfanyl-6-[5-methyl-1-(1-methyl-4-piperidyl)pyrazol-4-yl]pyrazolo[1,5-a]pyridine-3-carbonitrile (28 mg, 42% yield) as a white solid. LCMS (MM-ES+APCI, Pos): m/z 454.3 (M+H).

### Example 44

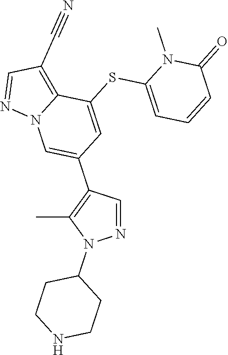 [SMILES: Cc1c(-c2cc(Sc3cccc(=O)n3C)c3c(C#N)cnn3c2)cnn1C1CCNCC1]

### 4-[(1-Methyl-6-oxo-2-pyridyl)sulfanyl]-6-[5-methyl-1-(4-piperidyl)pyrazol-4-yl]pyrazolo[1,5-a]pyridine-3-carbonitrile (44)

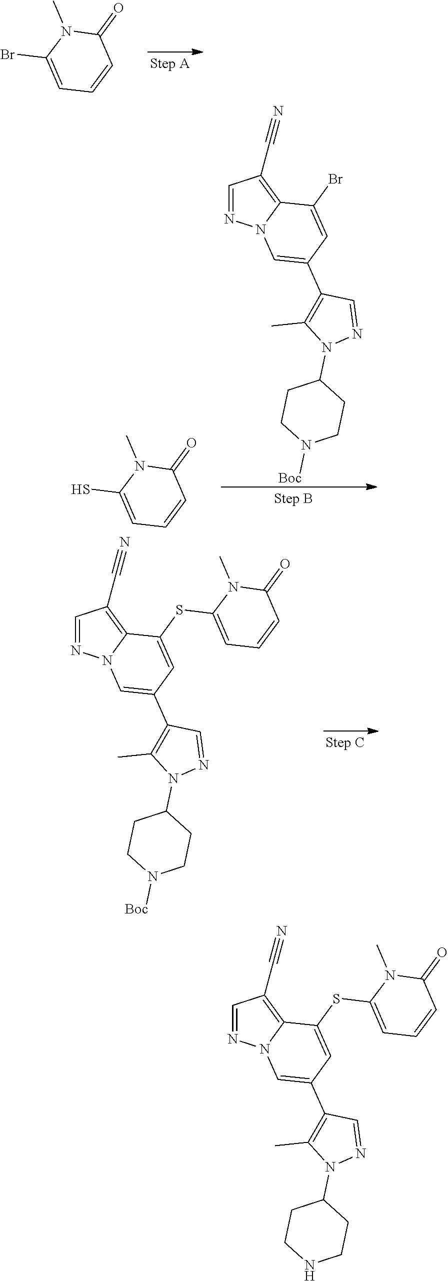

Step A. 1-Methyl-6-sulfanyl-pyridin-2-one. To a 10 mL round-bottom flask equipped with a magnetic stir bar was added 6-bromo-1-methyl-pyridin-2-one (0.15 g, 0.79 mmol) followed by the addition of DMF (2 mL) and H₂O (1 mL). Sodium hydrosulfide (90 mg, 1.6 mmol) was added into the mixture and stirred at 60° C. under an atmosphere of nitrogen for 1 h. Water (6 mL) was added to the reaction and the mixture was transferred to a separatory funnel. The aqueous layer was extracted with ethyl acetate (2 mL×3). The combined organic layers were concentrated under reduced pressure affording 1-methyl-6-sulfanyl-pyridin-2-one (0.28 g, crude product) as a yellow gum.

Step B. t-Butyl 4-[4-[3-cyano-4-[(1-methyl-6-oxo-2-pyridyl)sulfanyl]pyrazolo[1,5-a]pyridin-6-yl]-5-methyl-pyrazol-1-yl]piperidine-1-carboxylate. To a 10 mL round-bottom flask equipped with a magnetic stir bar was added tert-butyl 4-[4-(4-bromo-3-cyano-pyrazolo[1,5-a]pyridin-6-yl)-5-methyl-pyrazol-1-yl]piperidine-1-carboxylate (0.16 g, 0.33 mmol) and 1-methyl-6-sulfanyl-pyridin-2-one (0.28 g, 2.0 mmol) followed by the addition of DMSO (2 mL). DIEA (0.13 mL, 0.76 mmol) was added to the mixture. The flask was purged with nitrogen three times and stirred at 110° C. under an atmosphere of nitrogen for 16 h. Water (6 mL) was added to the reaction and the mixture was transferred to a separatory funnel. The aqueous layer mixture extracted with ethyl acetate (2 mL×3). The combined organic layers were washed with brine (2 mL), dried over anhydrous sodium sulfate, filtered, concentrated under reduced pressure and purified by preparative TLC (1:1 petroleum ether/ethyl acetate) to give tert-butyl 4-[4-[3-cyano-4-[(1-methyl-6-oxo-2-pyridyl)sulfanyl]pyrazolo[1,5-a]pyridin-6-yl]-5-methyl-pyrazol-1-yl]piperidine-1-carboxylate (80 mg, 45% yield) as a white solid. LCMS (MM-ES+APCI, Pos): m/z 546.4 (M+H).

Step C. 4-[(1-Methyl-6-oxo-2-pyridyl)sulfanyl]-6-[5-methyl-1-(4-piperidyl)pyrazol-4-yl]pyrazolo[1,5-a]pyridine-3-carbonitrile. Synthesized according to Example 1, Step B, substituting tert-butyl 4-[4-[3-cyano-4-[(1-methyl-6-oxo-2-pyridyl)sulfanyl]pyrazolo[1,5-a]pyridin-6-yl]-5-methyl-pyrazol-1-yl]piperidine-1-carboxylate for tert-butyl (3S)-3-[4-[3-cyano-4-(2-pyridylsulfanyl)pyrazolo[1,5-a]pyridin-6-yl]pyrazol-1-yl]piperidine-1-carboxylate to give 4-[(1-methyl-6-oxo-2-pyridyl)sulfanyl]-6-[5-methyl-1-(4-piperidyl)pyrazol-4-yl]pyrazolo[1,5-a]pyridine-3-carbonitrile (20 mg, 24% yield) as a white solid. LCMS (MM-ES+APCI, Pos): m/z 446.0 (M+H).

### Example 45

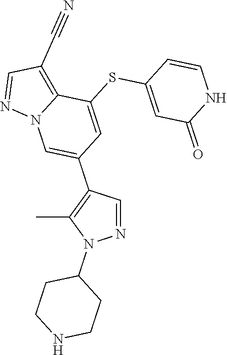 [SMILES: Cc1c(-c2cc(Sc3cc[nH]c(=O)c3)c3c(C#N)cnn3c2)cnn1C1CCNCC1]

### 6-[5-Methyl-1-(4-piperidyl) pyrazol-4-yl]-4-[(2-oxo-1H-pyridin-4-yl)sulfanyl] pyrazolo[1,5-a]pyridine-3-carbonitrile (45)

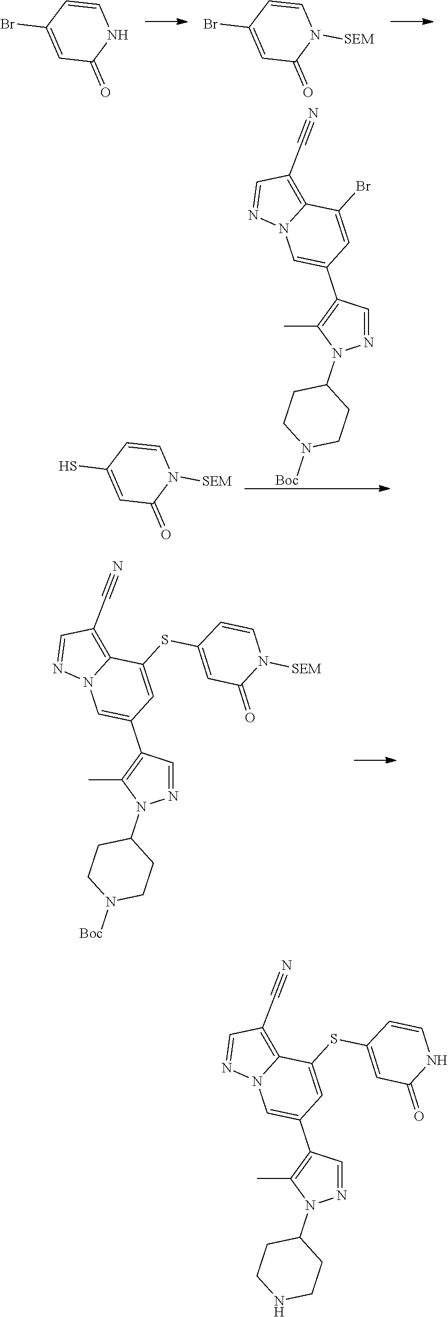

Step A. 4-Bromo-1-(2-trimethylsilylethoxymethyl)pyridin-2-one. Synthesized according to Example 7, Step A substituting 4-bromo-1H-pyridin-2-one for 7-bromo-1H-indazole to give 4-bromo-1-(2-trimethylsilylethoxymethyl)pyridin-2-one (0.32 g, 44% yield) as a yellow oil. LCMS (MM-ES+APCI, Pos): m/z 303.9 (M+H).

Step B. 4-Sulfanyl-1-(2-trimethylsilylethoxymethyl)pyridin-2-one. Synthesized according to Example 45, Step A substituting 4-bromo-1-(2-trimethylsilylethoxymethyl) pyridin-2-one for 6-bromo-1-methyl-pyridin-2-one to give 4-sulfanyl-1-(2-trimethylsilylethoxymethyl)pyridin-2-one (0.13 g, crude product) as a yellow solid. LCMS (MM-ES+APCI, Pos): m/z 258.0 (M+H).

Step C. t-Butyl 4-[4-[3-cyano-4-[[2-oxo-1-(2-trimethylsilylethoxymethyl)-4-pyridyl]sulfanyl]pyrazolo[1,5-a]pyridin-6-yl]-5-methyl-pyrazol-1-yl]piperidine-1-carboxylate. Synthesized according to Example 45, Step B substituting 4-sulfanyl-1-(2-trimethylsilylethoxymethyl)pyridin-2-one for 1-methyl-6-sulfanyl-pyridin-2-one to give tert-butyl 4-[4-[3-cyano-4-[[2-oxo-1-(2-trimethylsilylethoxymethyl)-4-pyridyl]sulfanyl]pyrazolo[1,5-a]pyridin-6-yl]-5-methyl-pyrazol-1-yl]piperidine-1-carboxylate (74 mg, 77% yield) as a colorless gum. LCMS (MM-ES+APCI, Pos): m/z 662.4 (M+H).

Step D. 6-[5-Methyl-1-(4-piperidyl) pyrazol-4-yl]-4-[(2-oxo-1H-pyridin-4-yl)sulfanyl] pyrazolo[1,5-a]pyridine-3-carbonitrile. Synthesized according to Example 11, Step B, substituting tert-butyl 4-[4-[3-cyano-4-[[2-oxo-1-(2-trimethylsilylethoxymethyl)-4-pyridyl]sulfanyl]pyrazolo[1,5-a]pyridin-6-yl]-5-methyl-pyrazol-1-yl]piperidine-1-carboxylate for tert-butyl (3S)-3-[4-[3-cyano-4-(2-fluorophenyl)sulfanyl-pyrazolo[1,5-a]pyridin-6-yl]pyrazol-1-yl]piperidine-1-carboxylate to give 6-[5-methyl-1-(4-piperidyl) pyrazol-4-yl]-4-[(2-oxo-1H-pyridin-4-yl)sulfanyl] pyrazolo[1,5-a]pyridine-3-carbonitrile (20 mg, 48% yield) as a white solid. LCMS (MM-ES+APCI, Pos): m/z 423.3 (M+H).

### Example 46

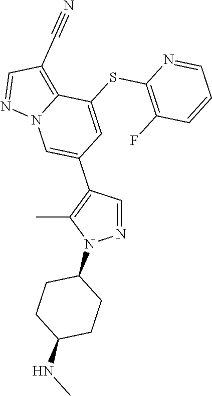 [SMILES: CN[C@H]1CC[C@@H](n2ncc(-c3cc(Sc4ncccc4F)c4c(C#N)cnn4c3)c2C)CC1]

### 4-[(3-Fluoro-2-pyridyl)sulfanyl]-6-[5-methyl-1-[4-(methylamino)cyclohexyl]pyrazol-4-yl]pyrazolo[1,5-a]pyridine-3-carbonitrile (46)

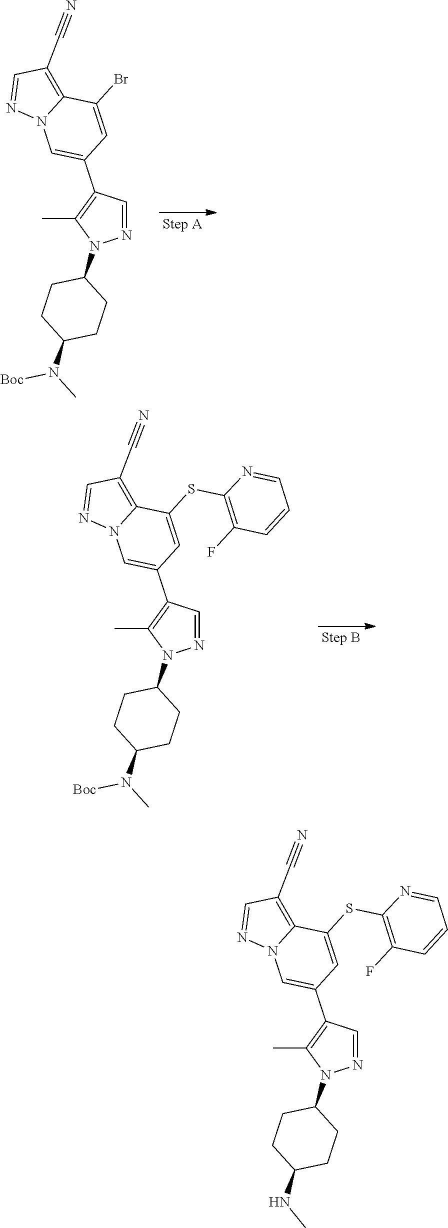 [SMILES: Cc1c(-c2cc(Br)c3c(C#N)cnn3c2)cnn1[C@H]1CC[C@@H](N(C)C(=O)OC(C)(C)C)CC1 ; Cc1c(-c2cc(Sc3ncccc3F)c3c(C#N)cnn3c2)cnn1[C@H]1CC[C@@H](N(C)C(=O)OC(C)(C)C)CC1 ; CN[C@H]1CC[C@@H](n2ncc(-c3cc(Sc4ncccc4F)c4c(C#N)cnn4c3)c2C)CC1]

Step A. t-Butyl N-[4-[4-[3-cyano-4-[(3-fluoro-2-pyridyl)sulfanyl]pyrazolo[1,5-a]pyridin-6-yl]-5-methyl-pyrazol-1-yl]cyclohexyl]-N-methyl-carbamate. Synthesized according to Experiment 1, Step A, substituting tert-butyl N-[4-[4-(4-bromo-3-cyano-pyrazolo[1,5-a]pyridin-6-yl)-5-methyl-pyrazol-1-yl]cyclohexyl]-N-methyl-carbamate for tert-butyl (3S)-3-[4-(4-bromo-3-cyano-pyrazolo[1,5-a]pyridin-6-yl)pyrazol-1-yl]piperidine-1-carboxylate to give tert-butyl N-[4-[4-[3-cyano-4-[(3-fluoro-2-pyridyl)sulfanyl]pyrazolo[1,5-a]pyridin-6-yl]-5-methyl-pyrazol-1-yl]cyclohexyl]-N-methyl-carbamate (90 mg, 82% yield) as a yellow solid. LCMS (MM-ES+APCI, Pos): m/z 562.2 (M+H).

Step B. 4-[(3-Fluoro-2-pyridyl)sulfanyl]-6-[5-methyl-1-[4-(methylamino)cyclohexyl]pyrazol-4-yl]pyrazolo[1,5-a]pyridine-3-carbonitrile. Synthesized according to Example 1, Step B, substituting tert-butyl N-[4-[4-[3-cyano-4-[(3-fluoro-2-pyridyl)sulfanyl]pyrazolo[1,5-a]pyridin-6-yl]-5-methyl-pyrazol-1-yl]cyclohexyl]-N-methyl-carbamate for tert-butyl (3S)-3-[4-[3-cyano-4-(2-pyridylsulfanyl)pyrazolo[1,5-a]pyridin-6-yl]pyrazol-1-yl]piperidine-1-carboxylate to give 4-[(3-fluoro-2-pyridyl)sulfanyl]-6-[5-methyl-1-[4-(methylamino)cyclohexyl]pyrazol-4-yl]pyrazolo[1,5-a]pyridine-3-carbonitrile (12 mg, 49% yield) as an off-white solid. LCMS (MM-ES+APCI, Pos): m/z 462.4 (M+H).

### Example 47

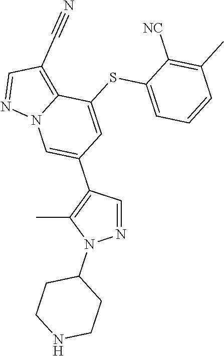 [SMILES: Cc1cccc(Sc2cc(-c3cnn(C4CCNCC4)c3C)cn3ncc(C#N)c23)c1C#N]

### 4-(2-Cyano-3-methyl-phenyl)sulfanyl-6-[5-methyl-1-(4-piperidyl)pyrazol-4-yl]pyrazolo[1,5-a]pyridine-3-carbonitrile (47)

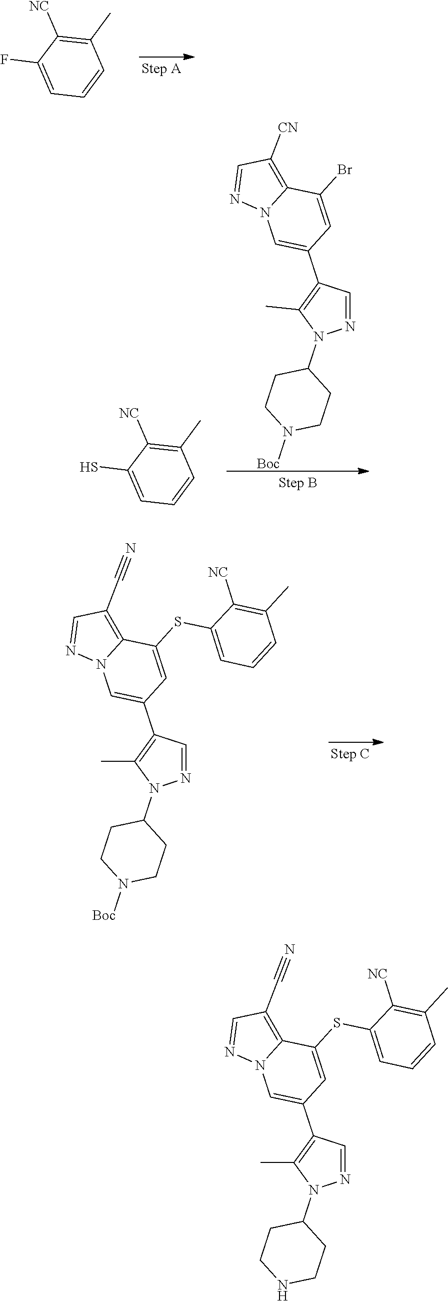

Step A. 2-Methyl-6-sulfanyl-benzonitrile. To a 40 mL vial equipped with a magnetic stir bar was added 2-fluoro-6-methyl-benzonitrile (0.30 g, 2.2 mmol) followed by the addition of DMF (5 mL) at 25° C. Sodium sulfide (0.12 mL, 2.4 mmol) was added and the mixture stirred at 25° C. for 19 hrs under an atmosphere of nitrogen. The reaction mixture was quenched with 1 M NaOH (10 mL). The mixture washed with DCM (10 mL×3). The pH of aqueous layer was adjusted to 1-2 with 12 M HCl and extracted with DCM (10 mL×3). The combined organic layer was washed with brine (10 mL×2), dried over anhydrous sodium sulfate, filtered, concentrated under reduced pressure and purified by silica gel chromatography (20-30% EtOAc/petroleum ether) to get 2-methyl-6-sulfanyl-benzonitrile (0.17 g, 51% yield) as a white solid.

Step B. t-Butyl 4-[4-[3-cyano-4-(2-cyano-3-methyl-phenyl)sulfanyl-pyrazolo[1,5-a]pyridin-6-yl]-5-methyl-pyrazol-1-yl]piperidine-1-carboxylate. To a 40 mL vial equipped with a magnetic stir bar were added tert-butyl 4-[4-(4-bromo-3-cyano-pyrazolo[1,5-a]pyridin-6-yl)-5-methyl-pyrazol-1-yl]piperidine-1-carboxylate (0.20 g, 0.41 mmol) and 2-methyl-6-sulfanyl-benzonitrile (73 mg, 0.49 mmol) followed by the addition of DMF (4 mL) at 25° C. K₂CO₃ (0.17 g, 1.2 mmol) was added and the reaction system purged with nitrogen for 2 mins and stirred at 110° ° C. for 18 hrs. Water (15 mL) was added to the reaction and the mixture was transferred to a separatory funnel. The mixture was extracted with ethyl acetate (15 mL×3). The combined organic layer was washed with brine (20 mL×2), dried over anhydrous sodium sulfate, filtered, concentrated under reduced pressure and purified by silica gel chromatography (10-50% EtOAc/petroleum ether) to get tert-butyl 4-[4-[3-cyano-4-(2-cyano-3-methyl-phenyl)sulfanyl-pyrazolo[1,5-a]pyridin-6-yl]-5-methyl-pyrazol-1-yl]piperidine-1-carboxylate (0.23 g, 99% yield) as a yellow solid. LCMS (MM-ES+APCI, Pos): m/z 554.4 (M+H).

Step C. 4-(2-Cyano-3-methyl-phenyl)sulfanyl-6-[5-methyl-1-(4-piperidyl)pyrazol-4-yl]pyrazolo[1,5-a]pyridine-3-carbonitrile. Synthesized according to Example 1, Step B, substituting tert-butyl 4-[4-[3-cyano-4-(2-cyano-3-methyl-phenyl)sulfanylpyrazolo[1,5-a]pyridin-6-yl]-5-methyl-pyrazol-1-yl]piperidine-1-carboxylate for tert-butyl (3S)-3-[4-[3-cyano-4-(2-pyridylsulfanyl)pyrazolo[1,5-a]pyridin-6-yl]pyrazol-1-yl]piperidine-1-carboxylate to give 4-(2-cyano-3-methyl-phenyl)sulfanyl-6-[5-methyl-1-(4-piperidyl)pyrazol-4-yl]pyrazolo[1,5-a]pyridine-3-carbonitrile (98 mg, 58% yield) as a white solid. LCMS (MM-ES+APCI, Pos): m/z 454.3 (M+H).

### Example 48

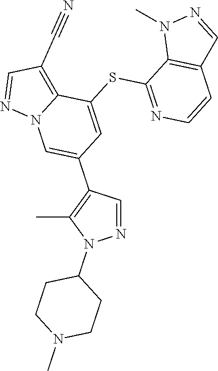 [SMILES: Cc1c(-c2cc(Sc3nccc4cnn(C)c34)c3c(C#N)cnn3c2)cnn1C1CCN(C)CC1]

### 6-[5-Methyl-1-(1-methyl-4-piperidyl)pyrazol-4-yl]-4-(1-methylpyrazolo[3,4-c]pyridin-7-yl)sulfanyl-pyrazolo[1,5-a]pyridine-3-carbonitrile (48)

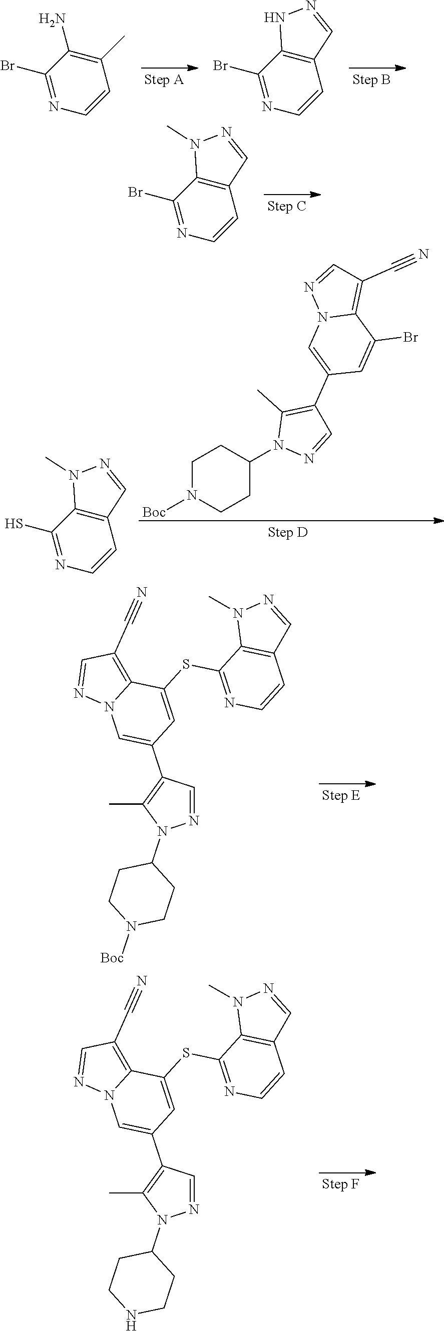

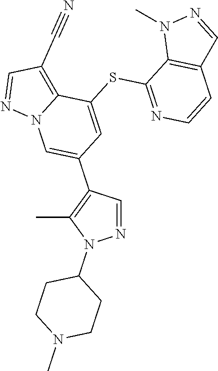 [SMILES: Cc1c(-c2cc(Sc3nccc4cnn(C)c34)c3c(C#N)cnn3c2)cnn1C1CCN(C)CC1]

Step A. 7-Bromo-1H-pyrazolo[3,4-c]pyridine. To a solution of 2-bromo-4-methyl-pyridin-3-amine (5.8 g, 31 mmol) in AcOH (50 mL) was added KOAc (4.0 g, 40 mmol) and NaNO₂ (4.5 g, 65 mmol) in water (14 mL) at 0° C. The mixture was stirred at 25° C. for 12 h. Aqueous NaHCO₃ was added to the mixture until the pH pf the solution was 8 and the aqueous layer was extracted with EtOAc (300 mL×3). The combined organic layer was washed with brine (50 mL), dried over Na₂SO₄, filtered, concentrated under reduced and purified by flash silica gel chromatography (0-30% EtOAc/petroleum ether) to give 7-bromo-1H-pyrazolo[3,4-c]pyridine (6.4 g, 98% yield) as a white solid. LCMS (MM-ES+APCI, Pos): m/z 198.0 (M+H).

Step B. 7-Bromo-1-methyl-pyrazolo[3,4-c]pyridine. To a stirred solution of 7-bromo-1H-pyrazolo[3,4-c]pyridine (6.4 g, 30 mmol) in DMF (65 mL) was added NaH (60%, 2.4 g, 61 mmol). The reaction mixture was stirred at 25° C. for 1 h. Mel (2.8 mL, 45.5 mmol) was added to the mixture and the reaction was stirred at 25° C. for 11 h. Water (200 mL) was added to the reaction and the mixture was transferred to a separatory funnel. The mixture was extracted with EtOAc (300 mL×3). The combined organic layer was washed with brine (50 mL), dried over Na₂SO₄, filtered, concentrated under reduced pressure and purified by silica gel chromatography (0-30% EtOAc/petroleum ether) to give 7-bromo-1-methyl-pyrazolo[3,4-c]pyridine (1.5 g, 23% yield) as a white solid. LCMS (MM-ES+APCI, Pos): m/z 212.1 (M+H).

Step C. 1-Methylpyrazolo[3,4-c]pyridine-7-thiol. To a solution of 7-bromo-1-methyl-pyrazolo[3,4-c]pyridine (0.70 g, 3.3 mmol) in EtOH (5 mL) and H₂O (1 mL) was added thiourea (0.37 g, 4.9 mmol). The mixture was stirred at 100° C. for 4 h. The reaction was filtered. The filtrate was concentrated under reduced pressure and purified by re-crystallization from EtOAc (15 mL) to give 1-methylpyrazolo[3,4-c]pyridine-7-thiol (0.44 g, 81% yield) as a yellow solid. LCMS (MM-ES+APCI, Pos): m/z 165.9 (M+H).

Step D. t-Butyl 4-[4-[3-cyano-4-(1-methylpyrazolo[3,4-c]pyridin-7-yl)sulfanyl-pyrazolo[1,5-a]pyridin-6-yl]-5-methyl-pyrazol-1-yl]piperidine-1-carboxylate. Synthesized according to Experiment 1, Step A, substituting tert-butyl 4-[4-(4-bromo-3-cyano-pyrazolo[1,5-a]pyridin-6-yl)-5-methyl-pyrazol-1-yl]piperidine-1-carboxylate for tert-butyl (3S)-3-[4-(4-bromo-3-cyano-pyrazolo[1,5-a]pyridin-6-yl)pyrazol-1-yl]piperidine-1-carboxylate to give tert-butyl 4-[4-[3-cyano-4-(1-methylpyrazolo[3,4-c]pyridin-7-yl)sulfanyl-pyrazolo[1,5-a]pyridin-6-yl]-5-methyl-pyrazol-1-yl]piperidine-1-carboxylate (80 mg, 35% yield) as a white solid. LCMS (MM-ES+APCI, Pos): m/z 570.3 (M+H).

Step E. 6-[5-Methyl-1-(4-piperidyl)pyrazol-4-yl]-4-(1-methylpyrazolo[3,4-c]pyridin-7-yl)sulfanylpyrazolo[1,5-a]pyridine-3-carbonitrile. Synthesized according to Example 1, Step B, substituting tert-butyl 4-[4-[3-cyano-4-(1-methylpyrazolo[3,4-c]pyridin-7-yl)sulfanyl-pyrazolo[1,5-a]pyridin-6-yl]-5-methyl-pyrazol-1-yl]piperidine-1-carboxylate for tert-butyl (3S)-3-[4-[3-cyano-4-(2-pyridylsulfanyl)pyrazolo[1,5-a]pyridin-6-yl]pyrazol-1-yl]piperidine-1-carboxylate to give 6-[5-methyl-1-(4-piperidyl)pyrazol-4-yl]-4-(1-methylpyrazolo[3,4-c]pyridin-7-yl)sulfanylpyrazolo[1,5-a]pyridine-3-carbonitrile (80 mg, crude product, HCl salt) as a white solid.

Step F. 6-[5-Methyl-1-(1-methyl-4-piperidyl)pyrazol-4-yl]-4-(1-methylpyrazolo[3,4-c]pyridin-7-yl)sulfanyl-pyrazolo[1,5-a]pyridine-3-carbonitrile. To a 8 mL vial equipped with a magnetic stir bar was added 6-[5-methyl-1-(4-piperidyl)pyrazol-4-yl]-4-(1-methylpyrazolo[3,4-c]pyridin-7-yl)sulfanyl-pyrazolo[1,5-a]pyridine-3-carbonitrile (70 mg, 0.14 mmol) followed by the addition of DCE (2 mL). Formaldehyde (37% in water, 0.21 mL, 2.8 mmol) and acetic acid (0.23 g, 0.83 mmol) were added into the mixture at 25° C. and stirred at 25° C. for 0.5 h. Sodium triacetoxyborohydride (88 mg, 0.41 mmol) was added and the mixture stirred at 25° C. for 0.5 h. The mixture was concentrated under reduced pressure and purified by preparative HPLC: (23%-53% acetonitrile/water (10 mM NH₄HCO₃). After lyophilization, 6-[5-methyl-1-(1-methyl-4-piperidyl)pyrazol-4-yl]-4-(1-methylpyrazolo[3,4-c]pyridin-7-yl)sulfanyl-pyrazolo[1,5-a]pyridine-3-carbonitrile (45 mg, 67% yield) was obtained as a white solid. LCMS (MM-ES+APCI, Pos): m/z 484.2 (M+H).

### Example 49

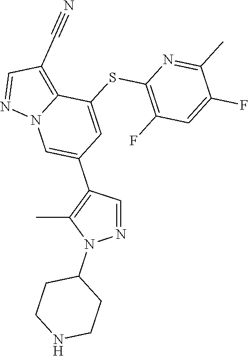 [SMILES: Cc1nc(Sc2cc(-c3cnn(C4CCNCC4)c3C)cn3ncc(C#N)c23)c(F)cc1F]

### 4-[(3,5-Difluoro-6-methyl-2-pyridyl)sulfanyl]-6-[5-methyl-1-(4-piperidyl)pyrazol-4-yl]pyrazolo[1,5-a]pyridine-3-carbonitrile (49)

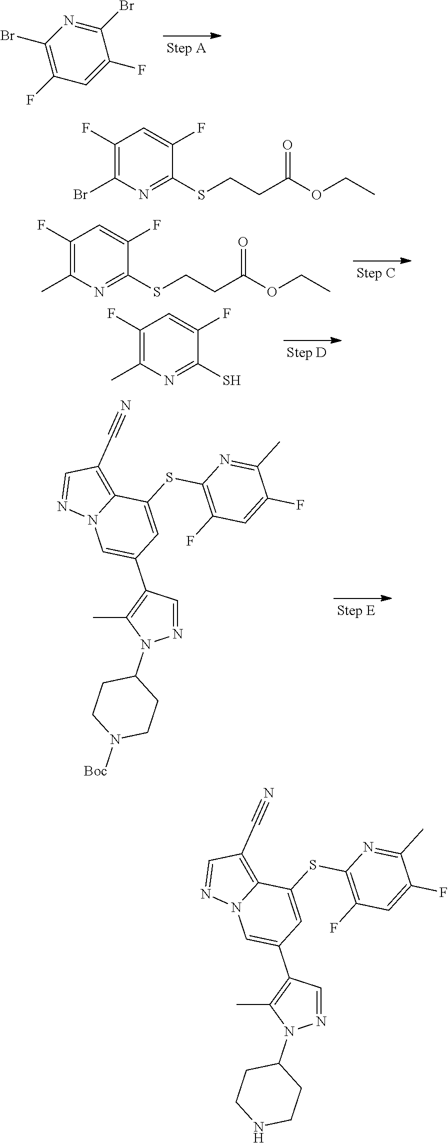

Step A. Ethyl 3-[(6-bromo-3,5-difluoro-2-pyridyl)sulfanyl]propanoate. To a 8 mL vial equipped with a magnetic stir bar were added 2,6-dibromo-3,5-difluoro-pyridine (0.30 g, 1.1 mmol) and ethyl 3-sulfanylpropanoate (0.16 g, 1.2 mmol) followed by the addition of dioxane (3 mL). Pd₂(dba)₃ (0.10 g, 0.11 mmol), xantphos (0.13 g, 0.21 mmol) and K₃PO₄ (0.45 g, 2.1 mmol) were added. The vial was purged with nitrogen and the mixture stirred at 90° ° C. for 1 h. Water (10 mL) was added to the reaction and the mixture was transferred to a separatory funnel. The mixture was extracted with ethyl acetate (15 mL×3). The combined organic layers were washed with brine (15 mL), dried over anhydrous sodium sulfate, filtered, concentrated under reduced and purified by silica gel chromatography (0-5% EtOAc/petroleum ether) to give ethyl 3-[(6-bromo-3,5-difluoro-2-pyridyl)sulfanyl]propanoate (0.26 g, 71% yield) as a yellow oil. LCMS (MM-ES+APCI, Pos): m/z 325.7 (M+H).

Step B. Ethyl 3-[(3,5-difluoro-6-methyl-2-pyridyl) sulfanyl] propanoate. To a 40 mL vial equipped with a magnetic stir bar were added ethyl 3-[(6-bromo-3,5-difluoro-2-pyridyl)sulfanyl]propanoate (0.24 g, 0.72 mmol) and 2,4,6-trimethyl-1,3,5,2,4,6-trioxatriborinane (1.0 mL, 3.6 mmol, 50% purity) followed by the addition of dioxane (5 mL). Pd(dppf)Cl₂·CH₂Cl₂ (60 mg, 0.074 mmol) and Cs₂CO₃ (0.48 g, 1.5 mmol) were added into the mixture at 25° C. The vial was purged with nitrogen and the mixture was stirred at 90° ° C. for 1 h. Water (15 mL) was added to the reaction and the mixture was transferred to a separatory funnel. The aqueous layer mixture was extracted with ethyl acetate (15 mL×3). The combined organic layers were washed with brine (15 mL), dried over anhydrous sodium sulfate, filtered, concentrated under reduced pressure and purified by silica gel chromatography (0-5% EtOAc/petroleum ether) to give ethyl 3-[(3,5-difluoro-6-methyl-2-pyridyl) sulfanyl] propanoate (0.15 g, 80% yield) as a yellow oil. LCMS (MM-ES+APCI, Pos): m/z 262.0 (M+H).

Step C. 3,5-Difluoro-6-methyl-pyridine-2-thiol. Synthesized according to Intermediate 3, Step B substituting ethyl 3-[(3,5-difluoro-6-methyl-2-pyridyl)sulfanyl]propanoate for tert-butyl (3S)-3-[4-[3-cyano-4-(3-ethoxy-3-oxo-propyl)sulfanylpyrazolo[1,5-a]pyridin-6-yl]pyrazol-1-yl]piperidine-1-carboxylate to give 3,5-difluoro-6-methyl-pyridine-2-thiol (60 mg, 97% yield) as a white solid.

Step D. t-Butyl 4-[4-[3-cyano-4-[(3,5-difluoro-6-methyl-2-pyridyl)sulfanyl]pyrazolo[1,5-a]pyridin-6-yl]-5-methyl-pyrazol-1-yl]piperidine-1-carboxylate. To a 8 mL vial equipped with a magnetic stir bar were added tert-butyl 4-[4-(4-bromo-3-cyano-pyrazolo[1,5-a]pyridin-6-yl)-5-methyl-pyrazol-1-yl]piperidine-1-carboxylate (0.12 g, 0.24 mmol) and 3,5-difluoro-6-methyl-pyridine-2-thiol (60 mg, 0.37 mmol) followed by the addition of DMSO (2 mL). DIEA (0.086 mL, 0.49 mmol) was added and the mixture stirred at 110° C. and for 16 h. Water (5 mL) was added to the reaction and the mixture was transferred to a separatory funnel. The aqueous layer mixture was extracted with ethyl acetate (5 mL×3). The combined organic layers were washed with brine (5 mL), dried over anhydrous sodium sulfate, filtered, concentrated under reduced pressure and purified by silica gel chromatography (30-50% EtOAc/petroleum ether) to give tert-butyl 4-[4-[3-cyano-4-[(3,5-difluoro-6-methyl-2-pyridyl)sulfanyl]pyrazolo[1,5-a]pyridin-6-yl]-5-methyl-pyrazol-1-yl]piperidine-1-carboxylate (0.13 g, 27% yield) as a yellow oil. LCMS (MM-ES+APCI, Pos): m/z 566.3 (M+H).

Step E. 4-[(3,5-Difluoro-6-methyl-2-pyridyl)sulfanyl]-6-[5-methyl-1-(4-piperidyl)pyrazol-4-yl]pyrazolo[1,5-a]pyridine-3-carbonitrile. Synthesized according to Example 1, Step B, substituting tert-butyl 4-[4-[3-cyano-4-[(3,5-difluoro-6-methyl-2-pyridyl)sulfanyl]pyrazolo[1,5-a]pyridin-6-yl]-5-methyl-pyrazol-1-yl]piperidine-1-carboxylate for tert-butyl (3S)-3-[4-[3-cyano-4-(2-pyridylsulfanyl)pyrazolo[1,5-a]pyridin-6-yl]pyrazol-1-yl]piperidine-1-carboxylate to give 4-[(3,5-difluoro-6-methyl-2-pyridyl)sulfanyl]-6-[5-methyl-1-(4-piperidyl)pyrazol-4-yl]pyrazolo[1,5-a]pyridine-3-carbonitrile (18 mg, 55% yield) as an off white solid. LCMS (MM-ES+APCI, Pos): m/z 466.1 (M+H).

### Example 50

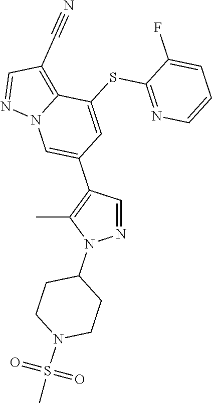 [SMILES: Cc1c(-c2cc(Sc3ncccc3F)c3c(C#N)cnn3c2)cnn1C1CCN(S(C)(=O)=O)CC1]

### 4-[(3-fluoro-2-pyridyl)sulfanyl]-6-[5-methyl-1-(1-methylsulfonyl-4-piperidyl)pyrazol-4-yl]pyrazolo[1,5-a]pyridine-3-carbonitrile (153)

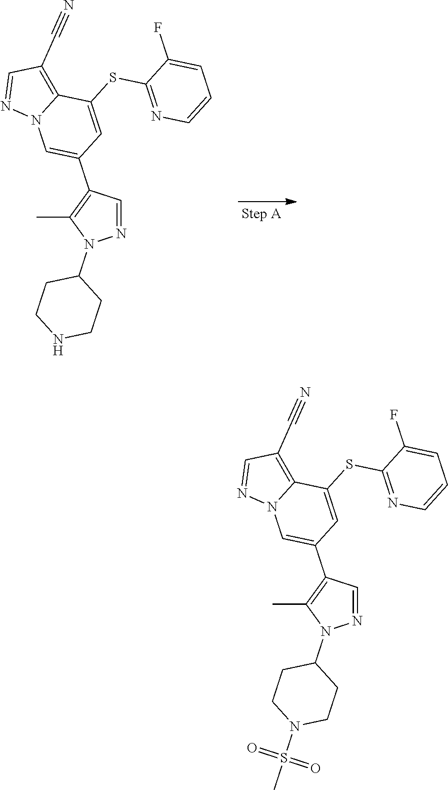 [SMILES: Cc1c(-c2cc(Sc3ncccc3F)c3c(C#N)cnn3c2)cnn1C1CCNCC1 ; Cc1c(-c2cc(Sc3ncccc3F)c3c(C#N)cnn3c2)cnn1C1CCN(S(C)(=O)=O)CC1]

Step A. 4-[(3-fluoro-2-pyridyl)sulfanyl]-6-[5-methyl-1-(1-methylsulfonyl-4-piperidyl)pyrazol-4-yl]pyrazolo[1,5-a]pyridine-3-carbonitrile. To 8 mL vial equipped with a magnetic stir bar was added 4-[(3-fluoro-2-pyridyl)sulfanyl]-6-[5-methyl-1-(4-piperidyl)pyrazol-4-yl]pyrazolo[1,5-a]pyridine-3-carbonitrile (50 mg, 0.11 mmol) followed by the addition of DCM (4 mL). The solution was cooled to 0° C., methylsulfonyl methanesulfonate (40 mg, 0.23 mmol) and TEA (0.07 mL, 0.49 mmol) were added. The mixture was allowed to warm to 25° C. and stir for 1 h. The mixture was concentrated and purified by preparative HPLC: (30%-60% acetonitrile/0.04% aqueous ammonia hydroxide+10 mM NH₄HCO₃). After lyophilization, 4-[(3-fluoro-2-pyridyl)sulfanyl]-6-[5-methyl-1-(1-methylsulfonyl-4-piperidyl)pyrazol-4-yl]pyrazolo[1,5-a]pyridine-3-carbonitrile (36.0 mg, 61% yield) was obtained as an off-white solid. LCMS (MM-ES+APCI, Pos): m/z 512.1 (M+H).

### Example 51

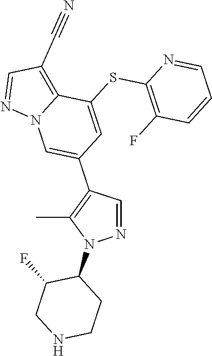 [SMILES: Cc1c(-c2cc(Sc3ncccc3F)c3c(C#N)cnn3c2)cnn1[C@H]1CCNC[C@@H]1F]

### 6-[1-[(3S,4S)-3-fluoro-4-piperidyl]-5-methyl-pyrazol-4-yl]-4-[(3-fluoro-2-pyridyl)sulfanyl]pyrazolo[1,5-a]pyridine-3-carbonitrile (154)

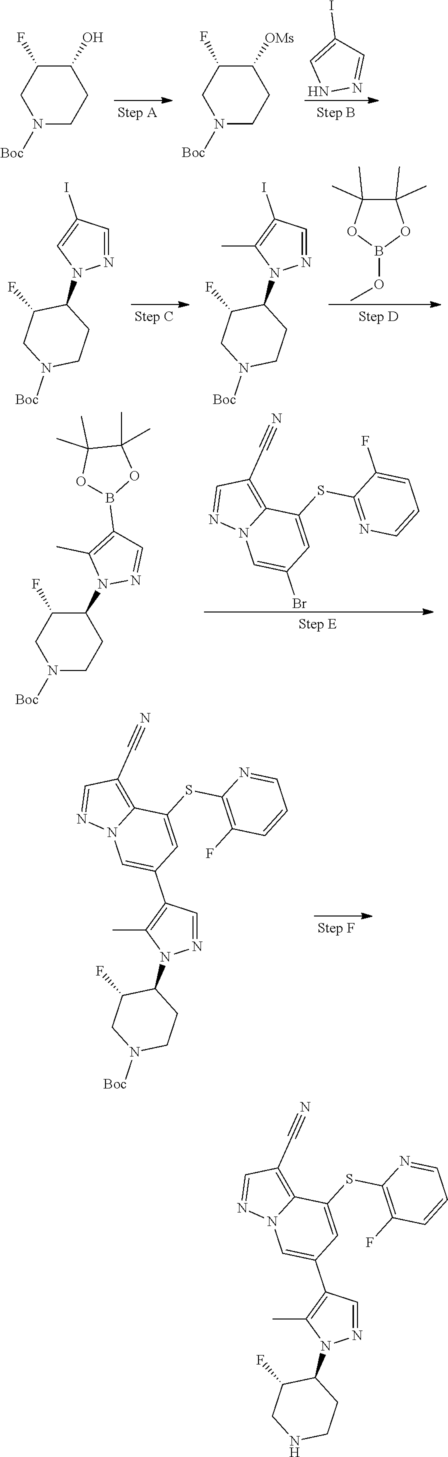

Step A. tert-butyl (3S,4R)-3-fluoro-4-methylsulfonyloxy-piperidine-1-carboxylate. To a solution of tert-butyl (3S,4R)-3-fluoro-4-hydroxy-piperidine-1-carboxylate (500 mg, 2.28 mmol) in DCM (8 mL) was added TEA (0.6 mL, 4.57 mmol) at 25° C. Then a solution of methylsulfonyl methanesulfonate (800 mg, 4.59 mmol) in DCM (8 mL) was added dropwise at 0° C. The resulting mixture was stirred at 25° C. for 1 hr. Water (10 mL) was added to the reaction and the mixture was transferred to a separatory funnel. The mixture was extracted with DCM (5 mL×3). The combined organic layers were washed with brine (15 mL), dried over anhydrous sodium sulfate, filtered and concentrated under reduced pressure to give tert-butyl (3S,4R)-3-fluoro-4-methylsulfonyloxy-piperidine-1-carboxylate (50 mg, 96% yield) as a yellow solid.

Step B. tert-butyl (3S,4S)-3-fluoro-4-(4-iodopyrazol-1-yl)piperidine-1-carboxylate. To a solution of tert-butyl (3S,4R)-3-fluoro-4-methylsulfonyloxy-piperidine-1-carboxylate (650 mg, 2.19 mmol) and 4-iodo-1H-pyrazole (430 mg, 2.22 mmol) in DMF (10 mL). To the mixture was added Cs₂CO₃ (1.42 g, 4.37 mmol) and stirred at 25-90° C. for 15 hr. Water was added to the reaction and the mixture was transferred to a separatory funnel. The mixture was extracted with ethyl acetate (5 mL×2). The combined organic layers were washed with brine (15 mL), dried over anhydrous sodium sulfate, filtered, concentrated and purified by column chromatography (SiO₂, 10-15% Petroleum ether in Ethyl acetate) to give tert-butyl (3S,4S)-3-fluoro-4-(4-iodopyrazol-1-yl)piperidine-1-carboxylate (450 mg, 48% yield) as a colorless oil. LCMS (MM-ES+APCI, Pos): m/z 340.0 (M+H).

Step C. tert-butyl (3S,4S)-3-fluoro-4-(4-iodo-5-methyl-pyrazol-1-yl)piperidine-1-carboxylate. To a 100 mL round-bottom flask equipped with a magnetic stir bar was added tert-butyl (3S,4S)-3-fluoro-4-(4-iodopyrazol-1-yl)piperidine-1-carboxylate (200 mg, 0.46 mmol) followed by the addition THF (4 mL). The solution was cooled to −65° C. LDA (2 M, 0.5 mL) was added dropwise. The mixture was stirred at −65° C. for 1 h. Mel (0.05 mL, 0.91 mmol) was added in portions, and the reaction stirred for 2 h. NH₄Cl (5 mL) was added to the reaction and the mixture was transferred to a separatory funnel. The mixture was extracted with ethyl acetate (5 mL×3). The combined organic layers were washed with brine (5 mL), dried over anhydrous sodium sulfate, filtered, concentrated and purified by silica gel column chromatography (30-60% ethyl acetate in petroleum ether) to give tert-butyl (3S,4S)-3-fluoro-4-(4-iodo-5-methyl-pyrazol-1-yl)piperidine-1-carboxylate (198 mg, 98% yield) as a colorless oil. LCMS (MM-ES+APCI, Pos): m/z 353.9 (M+H).

Step D. tert-butyl (3S,4S)-3-fluoro-4-[5-methyl-4-(4,4,5,5-tetramethyl-1,3,2-dioxaborolan-2-yl)pyrazol-1-yl]piperidine-1-carboxylate. To a 100 mL round-bottom flask equipped with a magnetic stir bar was added tert-butyl (3S,4S)-3-fluoro-4-(4-iodo-5-methyl-pyrazol-1-yl)piperidine-1-carboxylate (156.45 mg, 0.36 mmol) followed by the addition of THF (10 mL). i-PrMgCl (2 M, 0.36 mL) was added into the mixture at 25° C., and the mixture was stirred for 1 h at 25° C. 2-Methoxy-4,4,5,5-tetramethyl-1,3,2-dioxaborolane (231 mg, 1.46 mmol) was added into the mixture at 25° C. and stirred for 1 h. NH₄Cl (5 mL) was added to the reaction and the mixture was transferred to a separatory funnel. The mixture was extracted with ethyl acetate (10 mL×3). The combined organic layers were washed with brine (10 mL), dried over anhydrous sodium sulfate, filtered, concentrated and purified by silica gel column chromatography (30-60% ethyl acetate in petroleum ether) to give tert-butyl (3S,4S)-3-fluoro-4-[5-methyl-4-(4,4,5,5-tetramethyl-1,3,2-dioxaborolan-2-yl)pyrazol-1-yl]piperidine-1-carboxylate (140 mg, 76% yield) as a colorless oil.

Step E. tert-butyl (3S,4S)-4-[4-[3-cyano-4-[(3-fluoro-2-pyridyl)sulfanyl]pyrazolo[1,5-a]pyridin-6-yl]-5-methyl-pyrazol-1-yl]-3-fluoro-piperidine-1-carboxylate. To a 8 mL vial equipped with a magnetic stir bar were added tert-butyl (3S,4S)-3-fluoro-4-[5-methyl-4-(4,4,5,5-tetramethyl-1,3,2-dioxaborolan-2-yl)pyrazol-1-yl]piperidine-1-carboxylate (140 mg, 0.34 mmol), K₂CO₃ (101 mg, 0.73 mmol), Pd₂(dba)₃ (26 mg, 0.028 mmol) and XPhos (28 mg, 0.058.74 mmol) followed by the addition of 1,4-dioxane (5 mL) and H₂O (1 mL). 6-Bromo-4-[(3-fluoro-2-pyridyl)sulfanyl]pyrazolo[1,5-a]pyridine-3-carbonitrile (120 mg, 0.28 mmol) was added into the mixture at 25° C. The mixture was heated to 90° C. and stirred for 1 h. Water (10 mL) was added to the reaction and the mixture was transferred to a separatory funnel. The mixture was extracted with ethyl acetate (10 mL×3). The combined organic layers were washed with brine (10 mL), dried over anhydrous sodium sulfate, filtered, concentrated and purified by silica gel column chromatography (50-75% ethyl acetate in petroleum ether) to give tert-butyl (3S,4S)-4-[4-[3-cyano-4-[(3-fluoro-2-pyridyl)sulfanyl]pyrazolo[1,5-a]pyridin-6-yl]-5-methyl-pyrazol-1-yl]-3-fluoro-piperidine-1-carboxylate (126 mg, 70% yield) as a brown oil. LCMS (MM-ES+APCI, Pos): m/z 552.1 (M+H).

Step F. 6-[1-[(3S,4S)-3-fluoro-4-piperidyl]-5-methyl-pyrazol-4-yl]-4-[(3-fluoro-2-pyridyl)sulfanyl]pyrazolo[1,5-a]pyridine-3-carbonitrile. To a 50 mL round-bottom flask equipped with a magnetic stir bar was added tert-butyl (3S,4S)-4-[4-[3-cyano-4-[(3-fluoro-2-pyridyl)sulfanyl]pyrazolo[1,5-a]pyridin-6-yl]-5-methyl-pyrazol-1-yl]-3-fluoro-piperidine-1-carboxylate (115 mg, 0.18 mmol) followed by the addition of DCM (4 mL). Then HCl/dioxane (4 M, 4 mL) was added into the mixture at 25° C. and stirred at 25° C. for 1 h. The mixture was concentrated under reduced pressure affording the crude product as light yellow solid and purified by preparative HPLC (25%-55% acetonitrile/0.04% aqueous ammonia hydroxide+10 mM NH₄HCO₃). After lyophilization, 6-[1-[(3S,4S)-3-fluoro-4-piperidyl]-5-methyl-pyrazol-4-yl]-4-[(3-fluoro-2-pyridyl)sulfanyl]pyrazolo[1,5-a]pyridine-3-carbonitrile (43.4 mg, 51% yield) was obtained as a white solid. LCMS (MM-ES+APCI, Pos): m/z 453.1 (M+H).

### Example 52

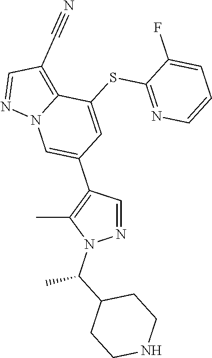 [SMILES: Cc1c(-c2cc(Sc3ncccc3F)c3c(C#N)cnn3c2)cnn1[C@@H](C)C1CCNCC1]

### 4-[(3-fluoro-2-pyridyl)sulfanyl]-6-[5-methyl-1-[(1S)-1-(4-piperidyl)ethyl]pyrazol-4-yl]pyrazolo[1,5-a]pyridine-3-carbonitrile (161)

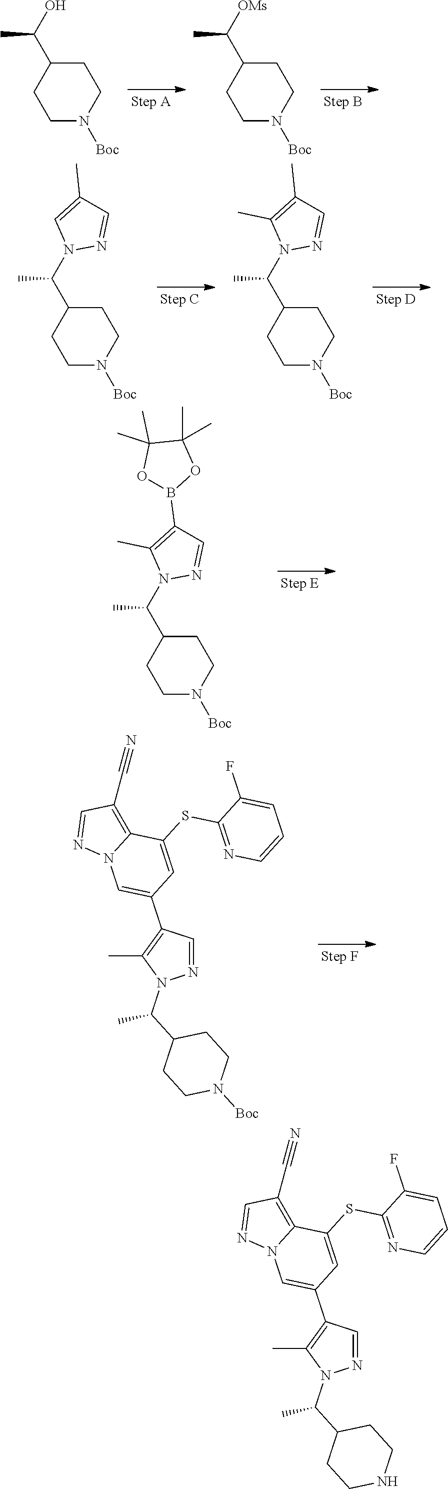

Step A. tert-butyl 4-[(1R)-1-methylsulfonyloxyethyl]piperidine-1-carboxylate. Synthesized according to Example 50, Step A, substituting 4-[(3-fluoro-2-pyridyl)sulfanyl]-6-[5-methyl-1-(4-piperidyl)pyrazol-4-yl]pyrazolo[1,5-a]pyridine-3-carbonitrile for tert-butyl 4-[(1R)-1-hydroxyethyl]piperidine-1-carboxylate to give tert-butyl 4-[(1R)-1-methylsulfonyloxyethyl]piperidine-1-carboxylate (1.43 g, crude product) as a yellow oil.

Step B. tert-butyl 4-[(1S)-1-(4-iodopyrazol-1-yl)ethyl]piperidine-1-carboxylate. Synthesized according to Example 51, Step B, substituting tert-butyl (3S,4R)-3-fluoro-4-methylsulfonyloxy-piperidine-1-carboxylate for tert-butyl 4-[(1R)-1-methylsulfonyloxyethyl]piperidine-1-carboxylate to give tert-butyl 4-[(1S)-1-(4-iodopyrazol-1-yl)ethyl]piperidine-1-carboxylate (1 g, 70% yield,) as a white solid. LCMS (MM-ES+APCI, Pos): m/z 350.1 (M-tBu+H).

Step C. tert-butyl 4-[(1S)-1-(4-iodo-5-methyl-pyrazol-1-yl)ethyl]piperidine-1-carboxylate. Synthesized according to Example 51, Step C, substituting tert-butyl (3S,4S)-3-fluoro-4-(4-iodopyrazol-1-yl)piperidine-1-carboxylate for tert-butyl 4-[(1S)-1-(4-iodopyrazol-1-yl)ethyl]piperidine-1-carboxylate to give tert-butyl 4-[(1S)-1-(4-iodo-5-methyl-pyrazol-1-yl)ethyl]piperidine-1-carboxylate (830 mg, 99% yield) as a colorless oil. LCMS (MM-ES+APCI, Pos): m/z 420.2 (M+H).

Step D. tert-butyl 4-[(1S)-1-[5-methyl-4-(4,4,5,5-tetramethyl-1,3,2-dioxaborolan-2-yl)pyrazol-1-yl]ethyl]piperidine-1-carboxylate. Synthesized according to Example 51, Step D, substituting tert-butyl (3S,4S)-3-fluoro-4-(4-iodo-5-methyl-pyrazol-1-yl)piperidine-1-carboxylate for tert-butyl-4-[(1S)-1-(4-iodo-5-methyl-pyrazol-1-yl)ethyl] piperidine-1-carboxylate to give tert-butyl 4-[(1S)-1-[5-methyl-4-(4,4,5,5-tetramethyl-1,3,2-dioxaborolan-2-yl)pyrazol-1-yl]ethyl]piperidine-1-carboxylate (800 mg, 79% yield) as a colorless oil.

Step E. tert-butyl 4-[(1S)-1-[4-[3-cyano-4-[(3-fluoro-2-pyridyl)sulfanyl]pyrazolo[1,5-a]pyridin-6-yl]-5-methylpyrazol-1-yl]ethyl]piperidine-1-carboxylate. Synthesized according to Example 51, Step E, substituting tert-butyl (3S,4S)-3-fluoro-4-[5-methyl-4-(4,4,5,5-tetramethyl-1,3,2-dioxaborolan-2-yl)pyrazol-1-yl]piperidine-1-carboxylate for tert-butyl 4-[(1S)-1-[5-methyl-4-(4,4,5,5-tetramethyl-1,3,2-dioxaborolan-2-yl)pyrazol-1-yl]ethyl]piperidine-1-carboxylate to give tert-butyl 4-[(1S)-1-[4-[3-cyano-4-[(3-fluoro-2-pyridyl)sulfanyl]pyrazolo[1,5-a]pyridin-6-yl]-5-methylpyrazol-1-yl]ethyl]piperidine-1-carboxylate (180 mg, 33% yield) as a yellow oil. LCMS (MM-ES+APCI, Pos): m/z 562.2 (M+H).

Step F. 4-[(3-fluoro-2-pyridyl)sulfanyl]-6-[5-methyl-1-[(1S)-1-(4-piperidyl)ethyl]pyrazol-4-yl]pyrazolo[1,5-a]pyridine-3-carbonitrile. Synthesized according to Example 51, Step F, substituting tert-butyl (3S,4S)-4-[4-[3-cyano-4-[(3-fluoro-2-pyridyl)sulfanyl]pyrazolo[1,5-a]pyridin-6-yl]-5-methyl-pyrazol-1-yl]-3-fluoro-piperidine-1-carboxylate for tert-butyl 4-[(1S)-1-[4-[3-cyano-4-[(3-fluoro-2-pyridyl)sulfanyl]pyrazolo[1,5-a]pyridin-6-yl]-5-methyl-pyrazol-1-yl]ethyl]piperidine-1-carboxylate to give 4-[(3-fluoro-2-pyridyl)sulfanyl]-6-[5-methyl-1-[(1S)-1-(4-piperidyl)ethyl]pyrazol-4-yl]pyrazolo[1,5-a]pyridine-3-carbonitrile (27.6 mg, 44% yield) as a white solid. LCMS (MM-ES+APCI, Pos): m/z 462.3 (M+H).

### Example 53

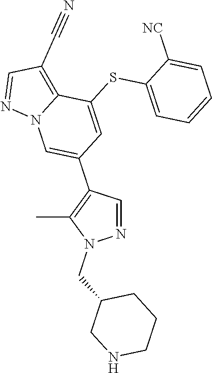 [SMILES: Cc1c(-c2cc(Sc3ccccc3C#N)c3c(C#N)cnn3c2)cnn1C[C@@H]1CCCNC1]

### 4-(2-cyanophenyl)sulfanyl-6-[5-methyl-1-[[(3R)-3-piperidyl]methyl]pyrazol-4-yl]pyrazolo[1,5-a]pyridine-3-carbonitrile (170)

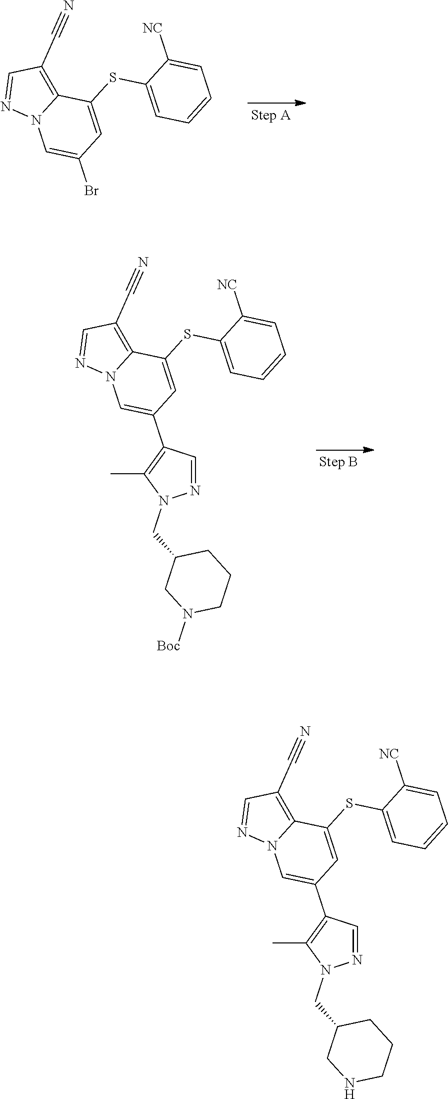 [SMILES: N#Cc1ccccc1Sc1cc(Br)cn2ncc(C#N)c12 ; Cc1c(-c2cc(Sc3ccccc3C#N)c3c(C#N)cnn3c2)cnn1C[C@@H]1CCCN(C(=O)OC(C)(C)C)C1 ; Cc1c(-c2cc(Sc3ccccc3C#N)c3c(C#N)cnn3c2)cnn1C[C@@H]1CCCNC1]

Step A. tert-butyl (3R)-3-[[4-[3-cyano-4-(2-cyanophenyl)sulfanyl-pyrazolo[1,5-a]pyridin-6-yl]-5-methyl-pyrazol-1-yl]methyl]piperidine-1-carboxylate. Synthesized according to Example 51, Step E, substituting tert-butyl (3S,4S)-3-fluoro-4-[5-methyl-4-(4,4,5,5-tetramethyl-1,3,2-dioxaborolan-2-yl)pyrazol-1-yl]piperidine-1-carboxylate for added tert-butyl (3R)-3-[5-methyl-4-(4,4,5,5-tetramethyl-1,3,2-dioxaborolan-2-yl)pyrazol-1-yl]methyl]piperidine-1-carboxylate to give tert-butyl (3R)-3-[[4-[3-cyano-4-(2-cyanophenyl)sulfanyl-pyrazolo[1,5-a]pyridin-6-yl]-5-methyl-pyrazol-1-yl]methyl]piperidine-1-carboxylate (150 mg, 58% yield) as a white solid. LCMS (MM-ES+APCI, Pos): m/z 554.4 (M+H).

Step B. 4-(2-cyanophenyl)sulfanyl-6-[5-methyl-1-[[(3R)-3-piperidyl]methyl]pyrazol-4-yl]pyrazolo[1,5-a]pyridine-3-carbonitrile. Synthesized according to Example 51, Step F, substituting tert-butyl (3S,4S)-4-[4-[3-cyano-4-[(3-fluoro-2-pyridyl)sulfanyl]pyrazolo[1,5-a]pyridin-6-yl]-5-methyl-pyrazol-1-yl]-3-fluoro-piperidine-1-carboxylate for tert-butyl 4-[(1S)-1-[4-[3-cyano-4-[(3-fluoro-2-pyridyl)sulfanyl]pyrazolo[1,5-a]pyridin-6-yl]-5-methyl-pyrazol-1-yl]ethyl]piperidine-1-carboxylate to give 4-(2-cyanophenyl)sulfanyl-6-[5-methyl-1-[[(3R)-3-piperidyl]methyl]pyrazol-4-yl]pyrazolo[1,5-a]pyridine-3-carbonitrile (14.8 mg, 14% yield) as a white solid. LCMS (MM-ES+APCI, Pos): m/z 454.1 (M+H).

### Example 54

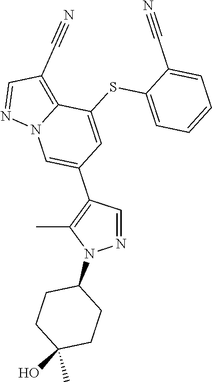 [SMILES: Cc1c(-c2cc(Sc3ccccc3C#N)c3c(C#N)cnn3c2)cnn1[C@H]1CC[C@@](C)(O)CC1]

### 4-(2-cyanophenyl)sulfanyl-6-[1-(4-hydroxy-4-methyl-cyclohexyl)-5-methyl-pyrazol-4-yl]pyrazolo[1,5-a]pyridine-3-carbonitrile (260)

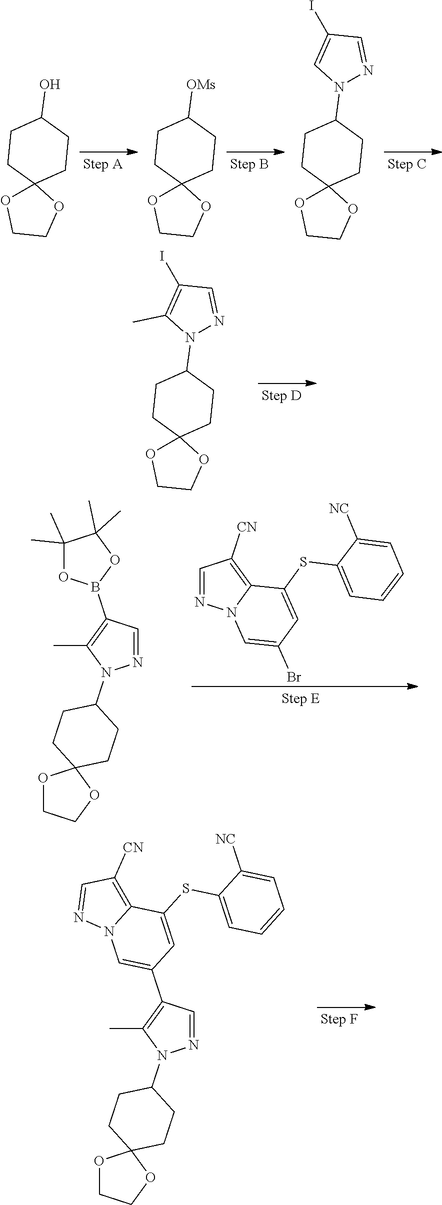

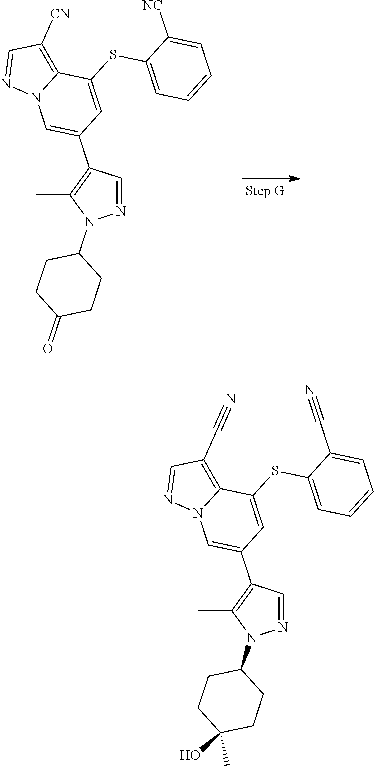 [SMILES: Cc1c(-c2cc(Sc3ccccc3C#N)c3c(C#N)cnn3c2)cnn1C1CCC(=O)CC1 ; Cc1c(-c2cc(Sc3ccccc3C#N)c3c(C#N)cnn3c2)cnn1[C@H]1CC[C@@](C)(O)CC1]

Step A. 1,4-dioxaspiro[4.5]decan-8-yl methanesulfonate. Synthesized according to Example 50, Step A, substituting 4-[(3-fluoro-2-pyridyl)sulfanyl]-6-[5-methyl-1-(4-piperidyl)pyrazol-4-yl]pyrazolo[1,5-a]pyridine-3-carbonitrile for 1,4-dioxaspiro[4.5]decan-8-ol to give 1,4-dioxaspiro[4.5]decan-8-yl methanesulfonate (22 g, 98%) as a yellow solid.

Step B. 1-(1,4-dioxaspiro[4.5]decan-8-yl)-4-iodo-pyrazole. Synthesized according to Example 51, Step B, substituting tert-butyl (3S,4R)-3-fluoro-4-methylsulfonyloxy-piperidine-1-carboxylate for 1,4-dioxaspiro[4.5]decan-8-yl methanesulfonate to give 1-(1,4-dioxaspiro[4.5]decan-8-yl)-4-iodo-pyrazole (5.2 g, 16% yield) as a white solid. LCMS (MM-ES+APCI, Pos): m/z 335.1 (M+H).

Step C. 1-(1,4-dioxaspiro[4.5]decan-8-yl)-4-iodo-5-methyl-pyrazole. Synthesized according to Example 51, Step C, substituting tert-butyl (3S,4S)-3-fluoro-4-(4-iodopyrazol-1-yl)piperidine-1-carboxylate for 1-(1,4-dioxaspiro[4.5]decan-8-yl)-4-iodo-pyrazole to give 1-(1,4-dioxaspiro[4.5]decan-8-yl)-4-iodo-5-methyl-pyrazole (6.7 g, 30% yield) as a white solid. LCMS (MM-ES+APCI, Pos): m/z 348.9 (M+H).

Step D. 1-(1,4-dioxaspiro[4.5]decan-8-yl)-5-methyl-4-(4,4,5,5-tetramethyl-1,3,2-dioxaborolan-2-yl)pyrazole. Synthesized according to Example 51, Step D, substituting tert-butyl (3S,4S)-3-fluoro-4-(4-iodo-5-methyl-pyrazol-1-yl)piperidine-1-carboxylate for 1-(1,4-dioxaspiro[4.5]decan-8-yl)-4-iodo-5-methyl-pyrazole to give 1-(1,4-dioxaspiro[4.5]decan-8-yl)-5-methyl-4-(4,4,5,5-tetramethyl-1,3,2-dioxaborolan-2-yl)pyrazole (1.5 g, 52% yield) as a white solid. LCMS (MM-ES+APCI, Pos): m/z 349.3 (M+H).

Step E. 4-(2-cyanophenyl)sulfanyl-6-[1-(1,4-dioxaspiro[4.5]decan-8-yl)-5-methyl-pyrazol-4-yl]pyrazolo[1,5-a]pyridine-3-carbonitrile. Synthesized according to Example 51, Step E, substituting tert-butyl (3S,4S)-3-fluoro-4-[5-methyl-4-(4,4,5,5-tetramethyl-1,3,2-dioxaborolan-2-yl)pyrazol-1-yl]piperidine-1-carboxylate for 1-(1,4-dioxaspiro[4.5]decan-8-yl)-5-methyl-4-(4,4,5,5-tetramethyl-1,3,2-dioxaborolan-2-yl)pyrazole to give 4-(2-cyanophenyl)sulfanyl-6-[1-(1,4-dioxaspiro[4.5]decan-8-yl)-5-methyl-pyrazol-4-yl]pyrazolo[1,5-a]pyridine-3-carbonitrile (386 mg, 37% yield) as a yellow solid. LCMS (MM-ES+APCI, Pos): m/z 497.2 (M+H).

Step F. 4-(2-cyanophenyl) sulfanyl-6-[5-methyl-1-(4-oxocyclohexyl)pyrazol-4-yl]pyrazolo[1,5-a]pyridine-3-carbonitrile. Synthesized according to Example 51, Step F, substituting tert-butyl (3S,4S)-4-[4-[3-cyano-4-[(3-fluoro-2-pyridyl)sulfanyl]pyrazolo[1,5-a]pyridin-6-yl]-5-methyl-pyrazol-1-yl]-3-fluoro-piperidine-1-carboxylate for 4-(2-cyanophenyl)sulfanyl-6-[1-(1,4-dioxaspiro[4.5]decan-8-yl)-5-methyl-pyrazol-4-yl]pyrazolo[1,5-a]pyridine-3-carbonitrile to give 4-(2-cyanophenyl) sulfanyl-6-[5-methyl-1-(4-oxocyclohexyl)pyrazol-4-yl]pyrazolo[1,5-a]pyridine-3-carbonitrile (220 mg, 50% yield) as a yellow solid. LCMS (MM-ES+APCI, Pos): m/z 453.3 (M+H).

Step G. 4-(2-cyanophenyl)sulfanyl-6-[1-(4-hydroxy-4-methyl-cyclohexyl)-5-methyl-pyrazol-4-yl]pyrazolo[1,5-a]pyridine-3-carbonitrile. To a vial equipped with a magnetic stir bar was added 4-(2-cyanophenyl)sulfanyl-6-[5-methyl-1-(4-oxocyclohexyl)pyrazol-4-yl]pyrazolo[1,5-a]pyridine-3-carbonitrile (200 mg, 0.34 mmol) followed by the addition of THF (4 mL). MeMgCl (3 M, 0.57 mL) was added drop wise at 25° C. The mixture was stirred at 25° C. for 10 min. The mixture was quenched by slow addition of saturated aqueous ammonium chloride (2 mL). The resulting mixture was transferred to a separatory funnel, and the aqueous layer mixture was extracted with ethyl acetate (5 mL×3). The combined organic layer was washed with brine (5 mL×2), dried over anhydrous sodium sulfate, filtered and purified by preparative HPLC (38%-68% acetonitrile/water (NH₄HCO₃). After lyophilization, 4-(2-cyanophenyl)sulfanyl-6-[1-(4-hydroxy-4-methyl-cyclohexyl)-5-methyl-pyrazol-4-yl]pyrazolo[1,5-a]pyridine-3-carbonitrile (15 mg, 9% yield) was obtained as an off-white solid. LCMS (MM-ES+APCI, Pos): m/z 469.3 (M+H).

### Example 55

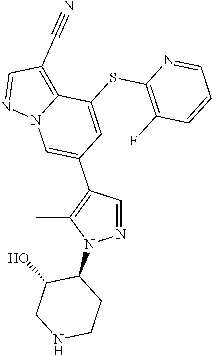 [SMILES: Cc1c(-c2cc(Sc3ncccc3F)c3c(C#N)cnn3c2)cnn1[C@H]1CCNC[C@@H]1O]

### 4-[(3-fluoro-2-pyridyl)sulfanyl]-6-[1-[(3S,4S)-3-hydroxy-4-piperidyl]-5-methylpyrazol-4-yl]pyrazolo[1,5-a]pyridine-3-carbonitrile (276)

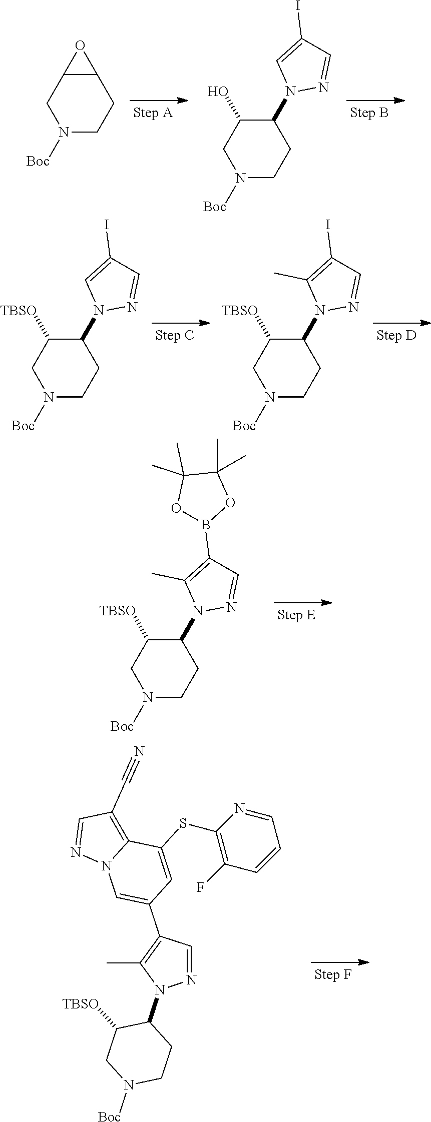

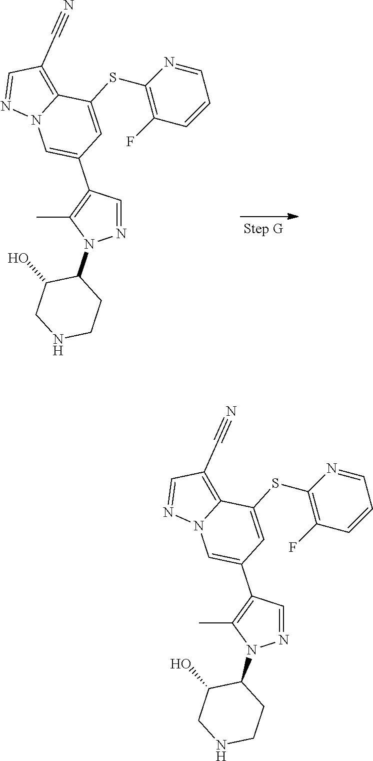 [SMILES: Cc1c(-c2cc(Sc3ncccc3F)c3c(C#N)cnn3c2)cnn1[C@H]1CCNC[C@@H]1O ; Cc1c(-c2cc(Sc3ncccc3F)c3c(C#N)cnn3c2)cnn1[C@H]1CCNC[C@@H]1O]

Step A. tert-butyl 3-hydroxy-4-(4-iodopyrazol-1-yl)piperidine-1-carboxylate. To a 100 mL three-necked round-bottom flask equipped with a magnetic stir bar was added tert-butyl 7-oxa-3-azabicyclo[4.1.0]heptane-3-carboxylate (2 g, 10.04 mmol) followed by the addition of THF (10 mL). The flask was then evacuated and backfilled with nitrogen three times. The solution was cooled to −78° C. LDA (2 M, 5.02 mL) was added while keeping internal temperature at −78° C. After addition, the mixture was stirred at this temperature for 3 h. 4-Iodo-1H-pyrazole (1.75 g, 9.03 mmol) in THF (10 mL) was added to mixture dropwise over 10 min. The mixture was stirred at 25° C. for 1 h and then heated to 70° C. and stirred for 16 h. Water (100 mL) was added to the reaction and the mixture was transferred to a separatory funnel. The mixture was extracted with ethyl acetate (40 mL×3). The combined organic layers were washed with brine (100 mL), dried over anhydrous sodium sulfate, filtered, concentrated and used into the next step without further purification. tert-Butyl 3-hydroxy-4-(4-iodopyrazol-1-yl)piperidine-1-carboxylate (8.2 g, crude) was obtained as a yellow oil. LCMS (MM-ES+APCI, Pos): m/z 338.0 (M+H).

Step B. Tert-butyl 3-[tert-butyl(dimethyl)silyl]oxy-4-(4-iodopyrazol-1-yl)piperidine-1-carboxylate. To a solution of tert-butyl 3-hydroxy-4-(4-iodopyrazol-1-yl)piperidine-1-carboxylate (6 g, 15.26 mmol) in DMF (30 mL) was added TBSCl (5.6 mL, 45.78 mmol) and imidazole (4.16 g, 61.03 mmol). The mixture was stirred at 25° C. for 16 h. Water (60 mL) was added to the reaction and the mixture was transferred to a separatory funnel. The mixture was extracted with ethyl acetate (30 mL×3). The combined organic layers were washed with brine (60 mL), dried over anhydrous sodium sulfate, filtered, concentrated and purified by flash silica gel chromatography (0-10% Ethyl acetate/Petroleum). Tert-butyl 3-[tert-butyl(dimethyl)silyl]oxy-4-(4-iodopyrazol-1-yl)piperidine-1-carboxylate (2 g, 25% yield) was obtained as a yellow solid. LCMS (MM-ES+APCI, Pos): m/z 452.2 (M+H).

Step C. Tert-butyl 3-[tert-butyl(dimethyl)silyl]oxy-4-(4-iodo-5-methyl-pyrazol-1-yl)piperidine-1-carboxylate. Synthesized according to Example 51, Step C, substituting tert-butyl (3S,4S)-3-fluoro-4-(4-iodopyrazol-1-yl)piperidine-1-carboxylate for tert-butyl 3-[tert-butyl(dimethyl)silyl]oxy-4-(4-iodopyrazol-1-yl)piperidine-1-carboxylate to give tert-butyl 3-[tert-butyl(dimethyl)silyl]oxy-4-(4-iodo-5-methyl-pyrazol-1-yl)piperidine-1-carboxylate (1.1 g, crude) was obtained as a yellow oil. LCMS (MM-ES+APCI, Pos): m/z 466.0 (M+H).

Step D. tert-butyl 3-[tert-butyl(dimethyl)silyl]oxy-4-[5-methyl-4-(4,4,5,5-tetramethyl-1,3,2-dioxaborolan-2-yl)pyrazol-1-yl]piperidine-1-carboxylate. Synthesized according to Example 51, Step D, substituting tert-butyl (3S,4S)-3-fluoro-4-(4-iodo-5-methyl-pyrazol-1-yl)piperidine-1-carboxylate for tert-butyl 3-[tert-butyl(dimethyl)silyl]oxy-4-(4-iodo-5-methyl-pyrazol-1-yl)piperidine-1-carboxylate to give tert-butyl 3-[tert-butyl(dimethyl)silyl]oxy-4-[5-methyl-4-(4,4,5,5-tetramethyl-1,3,2-dioxaborolan-2-yl)pyrazol-1-yl]piperidine-1-carboxylate (700 mg, crude) as a yellow solid, and used into the next step without further purification. LCMS (MM-ES+APCI, Pos): m/z 522.5 (M+H).

Step E. tert-butyl 3-[tert-butyl(dimethyl)silyl]oxy-4-[4-[3-cyano-4-[(3-fluoro-2-pyridyl)sulfanyl]pyrazolo[1,5-a]pyridin-6-yl]-5-methyl-pyrazol-1-yl]piperidine-1-carboxylate. Synthesized according to Example 51, Step E, substituting tert-butyl (3S,4S)-3-fluoro-4-[5-methyl-4-(4,4,5,5-tetramethyl-1,3,2-dioxaborolan-2-yl)pyrazol-1-yl]piperidine-1-carboxylate for added tert-butyl 3-[tert-butyl(dimethyl)silyl]oxy-4-[5-methyl-4-(4,4,5,5-tetramethyl-1,3,2-dioxaborolan-2-yl)pyrazol-1-yl]piperidine-1-carboxylate to give tert-butyl 3-[tert-butyl(dimethyl)silyl]oxy-4-[4-[3-cyano-4-[(3-fluoro-2-pyridyl)sulfanyl]pyrazolo[1,5-a]pyridin-6-yl]-5-methyl-pyrazol-1-yl]piperidine-1-carboxylate (180 mg, 16% yield) as a yellow solid. LCMS (MM-ES+APCI, Pos): m/z 664.4 (M+H).

Step F. 4-[(3-fluoro-2-pyridyl)sulfanyl]-6-[1-(3-hydroxy-4-piperidyl)-5-methyl-pyrazol-4-yl]pyrazolo[1,5-a]pyridine-3-carbonitrile. Synthesized according to Example 51, Step F, substituting tert-butyl (3S,4S)-4-[4-[3-cyano-4-[(3-fluoro-2-pyridyl)sulfanyl]pyrazolo[1,5-a]pyridin-6-yl]-5-methyl-pyrazol-1-yl]-3-fluoro-piperidine-1-carboxylate for tert-butyl 3-[tert-butyl(dimethyl)silyl]oxy-4-[4-[3-cyano-4-[(3-fluoro-2-pyridyl)sulfanyl]pyrazolo[1,5-a]pyridin-6-yl]-5-methyl-pyrazol-1-yl]piperidine-1-carboxylate to give 4-[(3-fluoro-2-pyridyl)sulfanyl]-6-[1-(3-hydroxy-4-piperidyl)-5-methyl-pyrazol-4-yl]pyrazolo[1,5-a]pyridine-3-carbonitrile (35 mg, 46% yield) as a white solid. LCMS (MM-ES+APCI, Pos): m/z 450.1 (M+H).

Step G. 4-[(3-fluoro-2-pyridyl)sulfanyl]-6-[1-[(3S,4S)-3-hydroxy-4-piperidyl]-5-methylpyrazol-4-yl]pyrazolo[1,5-a]pyridine-3-carbonitrile. 4-[(3-fluoro-2-pyridyl)sulfanyl]-6-[1-(3-hydroxy-4-piperidyl)-5-methyl-pyrazol-4-yl]pyrazolo[1,5-a]pyridine-3-carbonitrile (30 mg, 0.06 mmol,) was used for chiral resolution without purification. After lyophilization, 4-[(3-fluoro-2-pyridyl)sulfanyl]-6-[1-[(3S,4S)-3-hydroxy-4-piperidyl]-5-methylpyrazol-4-yl]pyrazolo[1,5-a]pyridine-3-carbonitrile (11.13 mg, 36% yield) was obtained as an off-white solid. LCMS (MM-ES+APCI, Pos): m/z 449.9 (M+H).

### Example 56

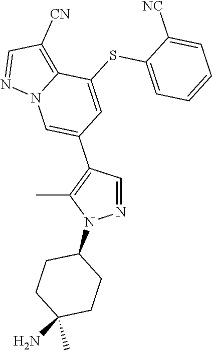 [SMILES: Cc1c(-c2cc(Sc3ccccc3C#N)c3c(C#N)cnn3c2)cnn1[C@H]1CC[C@@](C)(N)CC1]

### 6-[1-(4-amino-4-methyl-cyclohexyl)-5-methyl-pyrazol-4-yl]-4-(2-cyanophenyl) sulfanylpyrazolo[1,5-a]pyridine-3-carbonitrile (297)

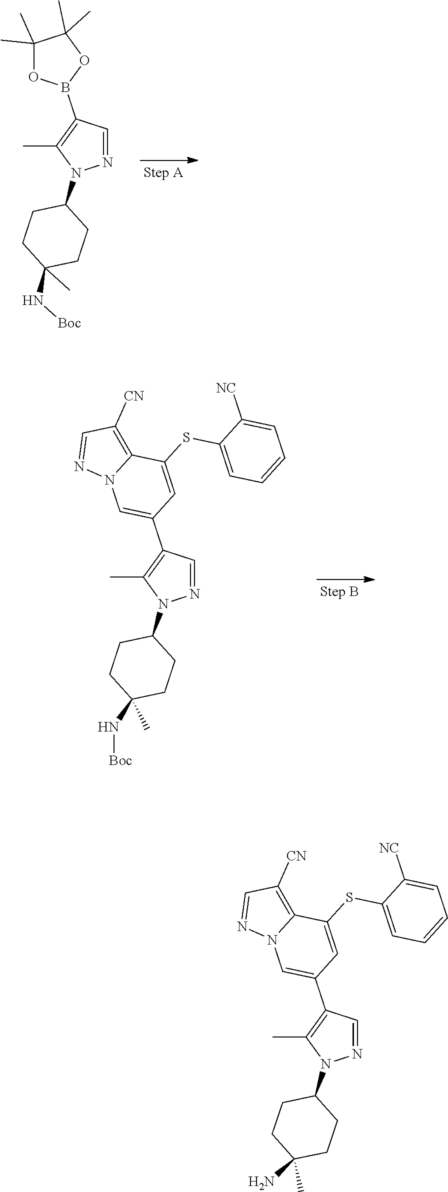 [SMILES: Cc1c(B2OC(C)(C)C(C)(C)O2)cnn1[C@H]1CC[C@@](C)(NC(=O)OC(C)(C)C)CC1 ; Cc1c(-c2cc(Sc3ccccc3C#N)c3c(C#N)cnn3c2)cnn1[C@H]1CC[C@@](C)(NC(=O)OC(C)(C)C)CC1 ; Cc1c(-c2cc(Sc3ccccc3C#N)c3c(C#N)cnn3c2)cnn1[C@H]1CC[C@@](C)(N)CC1]

Step A. tert-butyl N-[4-[4-[3-cyano-4-(2-cyanophenyl) sulfanyl-pyrazolo[1,5-a]pyridin-6-yl]-5-methylpyrazol-1-yl]-1-methyl-cyclohexyl] carbamate. Synthesized according to Example 51, Step E, substituting tert-butyl (3S,4S)-3-fluoro-4-[5-methyl-4-(4,4,5,5-tetramethyl-1,3,2-dioxaborolan-2-yl)pyrazol-1-yl]piperidine-1-carboxylate for tert-butyl N-[1-methyl-4-[5-methyl-4-(4,4,5,5-tetramethyl-1,3,2-dioxaborolan-2-yl) pyrazol-1-yl] cyclohexyl] carbamate to give tert-butyl N-[4-[4-[3-cyano-4-(2-cyanophenyl) sulfanyl-pyrazolo[1,5-a]pyridin-6-yl]-5-methylpyrazol-1-yl]-1-methyl-cyclohexyl] carbamate (200 mg, 79% yield) as a brown solid. LCMS (MM-ES+APCI, Pos): m/z 568.3 (M+H).

Step B. 6-[1-(4-amino-4-methyl-cyclohexyl)-5-methyl-pyrazol-4-yl]-4-(2-cyanophenyl) sulfanylpyrazolo[1,5-a]pyridine-3-carbonitrile. Synthesized according to Example 51, Step F, substituting tert-butyl (3S,4S)-4-[4-[3-cyano-4-[(3-fluoro-2-pyridyl)sulfanyl]pyrazolo[1,5-a]pyridin-6-yl]-5-methyl-pyrazol-1-yl]-3-fluoro-piperidine-1-carboxylate for tert-butyl N-[4-[4-[3-cyano-4-(2-cyanophenyl) sulfanyl-pyrazolo[1,5-a]pyridin-6-yl]-5-methyl-pyrazol-1-yl]-1-methyl-cyclohexyl] carbamate to give 6-[1-(4-amino-4-methyl-cyclohexyl)-5-methyl-pyrazol-4-yl]-4-(2-cyanophenyl) sulfanylpyrazolo[1,5-a]pyridine-3-carbonitrile (27 mg, 55% yield) as a white solid. LCMS (MM-ES+APCI, Pos): m/z 468.4 (M+H).

### Example 57

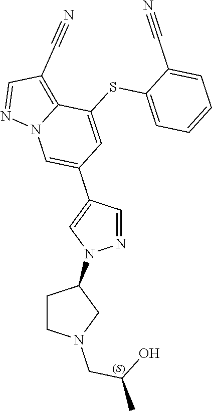

### 4-(2-cyanophenyl)sulfanyl-6-[1-[(3R)-1-[(2S)-2-hydroxypropyl]pyrrolidin-3-yl]pyrazol-4-yl]pyrazolo[1,5-a]pyridine-3-carbonitrile (169)

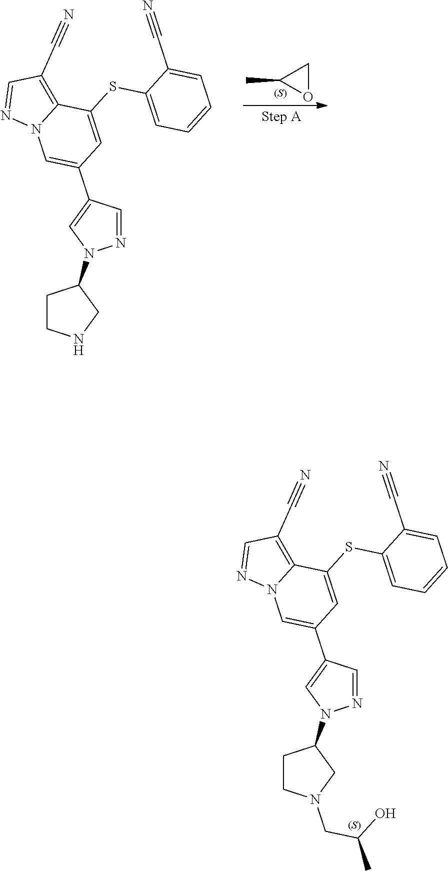

Step A. 4-(2-cyanophenyl)sulfanyl-6-[1-[(3R)-1-[(2S)-2-hydroxypropyl]pyrrolidin-3-yl]pyrazol-4-yl]pyrazolo[1,5-a]pyridine-3-carbonitrile. To a 50 mL Schlenk tube equipped with a magnetic stir bar was added 4-(2-cyanophenyl)sulfanyl-6-[1-[(3R)-pyrrolidin-3-yl]pyrazol-4-yl]pyrazolo[1,5-a]pyridine-3-carbonitrile (100 mg, 0.22 mmol) and (2S)-2-methyloxirane (0.03 mL 0.44 mmol) followed by the addition of EtOH (5 mL). DIEA (0.08 mL, 0.44 mmol) was added into the mixture at 25° C. The mixture was stirred at 90° C. for 2 h. Water (20 mL) was added to the reaction and the mixture was transferred to a separatory funnel. The mixture was extracted with ethyl acetate (20 mL×3). The combined organic layers were washed with brine (20 mL), dried over anhydrous sodium sulfate, filtered, concentrated and purified by preparative HPLC (30%-60% acetonitrile/10 mM NH₄HCO₃). After lyophilization, 4-(2-cyanophenyl)sulfanyl-6-[1-[(3R)-1-[(2S)-2-hydroxypropyl]pyrrolidin-3-yl]pyrazol-4-yl]pyrazolo[1,5-a]pyridine-3-carbonitrile (29.6 mg, 27% yield) was obtained as an off-white solid. LCMS (MM-ES+APCI, Pos): m/z 470.2 (M+H).

### Example 58

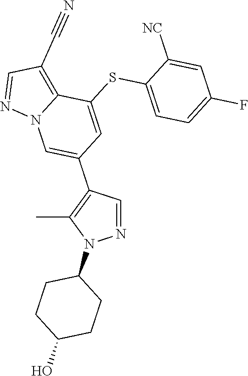 [SMILES: Cc1c(-c2cc(Sc3ccc(F)cc3C#N)c3c(C#N)cnn3c2)cnn1[C@H]1CC[C@H](O)CC1]

### 4-(2-cyano-4-fluoro-phenyl)sulfanyl-6-[1-(4-hydroxycyclohexyl)-5-methyl-pyrazol-4-yl]pyrazolo[1,5-a]pyridine-3-carbonitrile (275)

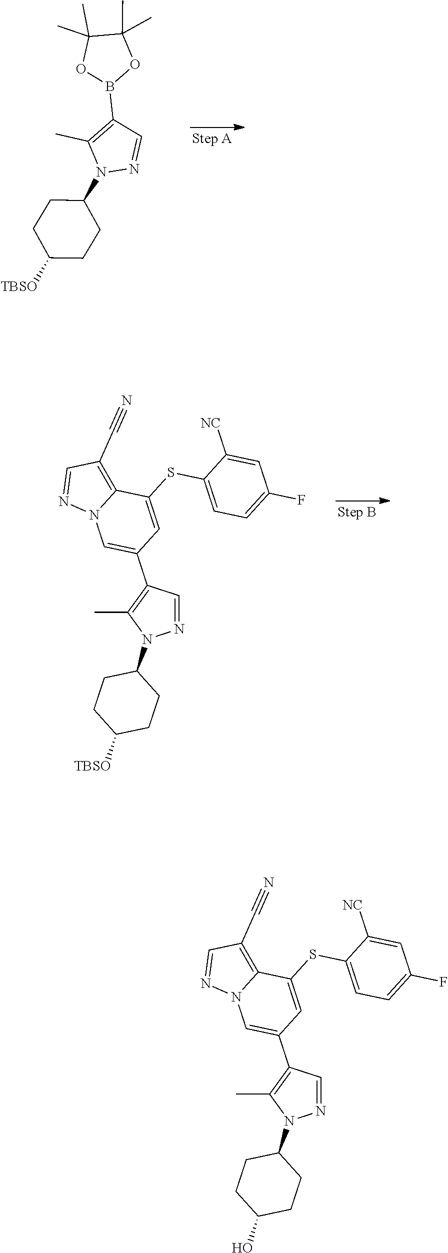 [SMILES: Cc1c(B2OC(C)(C)C(C)(C)O2)cnn1[C@H]1CC[C@H](O[Si](C)(C)C(C)(C)C)CC1 ; Cc1c(-c2cc(Sc3ccc(F)cc3C#N)c3c(C#N)cnn3c2)cnn1[C@H]1CC[C@H](O[Si](C)(C)C(C)(C)C)CC1 ; Cc1c(-c2cc(Sc3ccc(F)cc3C#N)c3c(C#N)cnn3c2)cnn1[C@H]1CC[C@H](O)CC1]

Step A. 6-[1-[4-[tert-butyl(dimethyl)silyl]oxycyclohexyl]-5-methyl-pyrazol-4-yl]-4-(2-cyano-4-fluorophenyl)sulfanyl-pyrazolo[1,5-a]pyridine-3-carbonitrile. Synthesized according to Example 51, Step E, substituting tert-butyl (3S,4S)-3-fluoro-4-[5-methyl-4-(4,4,5,5-tetramethyl-1,3,2-dioxaborolan-2-yl)pyrazol-1-yl]piperidine-1-carboxylate for tert-butyl-dimethyl-[4-[5-methyl-4-(4,4,5,5-tetramethyl-1,3,2-dioxaborolan-2-yl)pyrazol-1-yl]cyclohexoxy] silane to give 6-[1-[4-[tert-butyl(dimethyl)silyl]oxycyclohexyl]-5-methyl-pyrazol-4-yl]-4-(2-cyano-4-fluorophenyl)sulfanyl-pyrazolo[1,5-a]pyridine-3-carbonitrile (176 mg, 78% yield) as a white solid. LCMS (MM-ES+APCI, Pos): m/z 587.3 (M+H).

Step B. 4-(2-cyano-4-fluoro-phenyl)sulfanyl-6-[1-(4-hydroxycyclohexyl)-5-methyl-pyrazol-4-yl]pyrazolo[1,5-a]pyridine-3-carbonitrile. Synthesized according to Example 51, Step F, substituting tert-butyl (3S,4S)-4-[4-[3-cyano-4-[(3-fluoro-2-pyridyl)sulfanyl]pyrazolo[1,5-a]pyridin-6-yl]-5-methyl-pyrazol-1-yl]-3-fluoro-piperidine-1-carboxylate for 6-[1-[4-[tertbutyl(dimethyl)silyl]oxycyclohexyl]-5-methyl-pyrazol-4-yl]-4-(2-cyano-4-fluoro-phenyl)sulfanyl-pyrazolo[1,5-a]pyridine-3-carbonitrile to give −(2-cyano-4-fluoro-phenyl)sulfanyl-6-[1-(4-hydroxycyclohexyl)-5-methyl-pyrazol-4-yl]pyrazolo[1,5-a]pyridine-3-carbonitrile (56.5 mg, 51% yield) as a white solid. LCMS (MM-ES+APCI, Pos): m/z 473.3 (M+H).

### Example 59

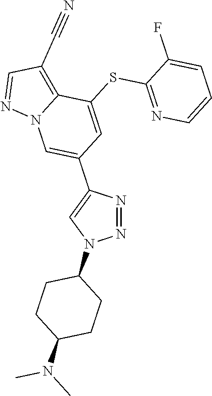 [SMILES: CN(C)[C@H]1CC[C@@H](n2cc(-c3cc(Sc4ncccc4F)c4c(C#N)cnn4c3)nn2)CC1]

### 6-[1-[4-(dimethylamino) cyclohexyl] triazol-4-yl]-4-[(3-fluoro-2-pyridyl) sulfanyl] pyrazolo [1,5-a]pyridine-3-carbonitrile (392)

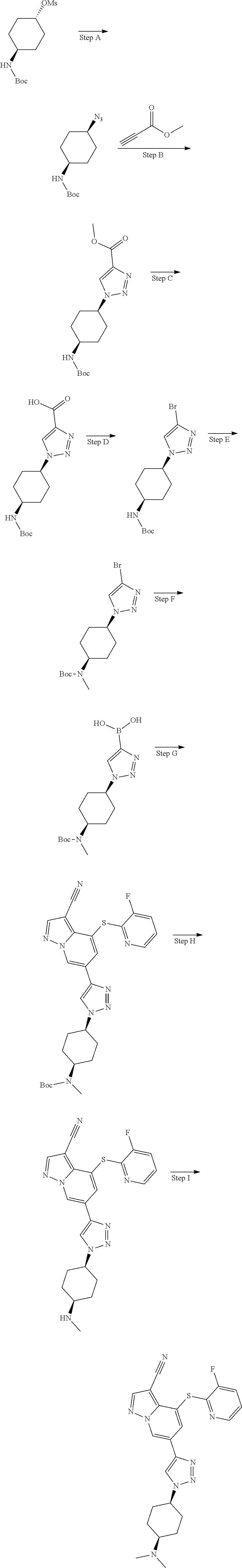

Step A. tert-butyl N-(4-azidocyclohexyl) carbamate. To a solution of [4-(tert-butoxycarbonylaminocyclohexyl] methanesulfonate (11.6 g, 39.5 mmol) in DMSO (150 mL) was added NaN₃ (6.91 g, 106 mmol). The mixture was stirred at 65° C. for 16 h. After cooling to 25° C., water (200 mL) was added to the reaction and the pH of mixture was adjusted to 9 by using saturated aqueous sodium bicarbonate. The mixture was transferred to a separatory funnel, extracted with ethyl acetate (150 mL×3). The combined organic layers were washed with brine (200 mL), dried over anhydrous sodium sulfate, filtered, concentrated and concentrated to about ¾ of the original volume to give a solution of tert-butyl N-(4-azidocyclohexyl) carbamate (19 g, crude) in THF (38 mL). The solution of crude product was used into the next step without further purification.

Step B. Methyl 1-[4-(tert-butoxycarbonylamino) cyclohexyl] triazole-4-carboxylate. A solution of tert-butyl N-(4-azidocyclohexyl) carbamate 18 mL, 37.45 mmol), CuI (7.13 g, 37.45 mmol) and DIEA (9.8 mL, 56.18 mmol) in THF (100 mL) was purged with nitrogen. Methyl prop-2-ynoate (4.7 mL, 56.2 mmol) was added into the mixture at 25° C. under N₂ atmosphere and stirred at 25° ° C. for 16 h. The mixture was added to MeOH (100 mL) and stirred at 25° ° C. for 0.5 h. The suspension was filtered through a pad of Celite and eluted with MeOH (20 mL). The filtrate was concentrated under reduced pressure affording the crude product and purified by recrystallization from ethyl acetate (30 mL) at 25° C. for 0.5 h. After filtration and drying under vacuum, the desired product methyl 1-[4-(tert-butoxycarbonylamino) cyclohexyl] triazole-4-carboxylate (15.6 g, crude) was obtained as a yellow solid.

Step C. 1-((1s,4s)-4-((tert-butoxycarbonyl)amino)cyclohexyl)-1H-1,2,3-triazole-4-carboxylic acid. To a solution of methyl 1-[4-(tert-butoxycarbonylamino) cyclohexyl] triazole-4-carboxylate (10 g, 30.83 mmol) in H₂O (100 mL) was added KOH (5.19 g, 92.49 mmol). The mixture was stirred at 50° C. for 16 h. The reaction mixture gave 1-((1s,4s)-4-((tert-butoxycarbonyl)amino)cyclohexyl)-1H-1,2,3-triazole-4-carboxylic acid and was used into the next step without further work up and purification. LCMS (MM-ES+APCI, Pos): m/z 255.2 (M+H).

Step D. Tert-butyl N-[4-(4-bromotriazol-1-yl) cyclohexyl] carbamate. To a solution of 1-[4-(tert-butoxycarbonylamino)cyclohexyl]triazole-4-carboxylic acid (5.00 g, 16.1 mmol) in H₂O (50 mL) was added KOH (2.71 g, 48.3 mmol) and molecular bromine (2.49 mL, 48.3 mmol). The mixture was stirred at 30° C. for 16 h. The suspension was filtered and the filter cake was washed with H₂O (10 mL). The solid was concentrated and purified by silica gel column chromatography (10-50% petroleum ether/ethyl acetate) to give tert-butyl N-[4-(4-bromotriazol-1-yl) cyclohexyl] carbamate (2.1 g, 37% yield) as a white solid.

Step E. tert-butyl N-[4-(4-bromotriazol-1-yl) cyclohexyl]-N-methyl-carbamate. A solution of tert-butyl N-[4-(4-bromotriazol-1-yl) cyclohexyl] carbamate (1.50 g, 4.31 mmol) in THF (10 mL) was degassed and purged with N² 3 times. 60% NaH (517 mg, 12.9 mmol) was added into the mixture at 0° C. After addition, the mixture was stirred at this temperature for 2 h. CH₃₁ (0.53 mL, 8.62 mmol) was added dropwise, the mixture was heated to 40° C. and stirred for 2 h. Water (50 mL) was added to the reaction and the mixture was transferred to a separatory funnel. The mixture was extracted with ethyl acetate (20 mL×3). The combined organic layers were washed with brine (50 mL), dried over anhydrous sodium sulfate, filtered, concentrated and purified by silica gel column chromatography (10-50% petroleum ether/ethyl acetate) to give tert-butyl N-[4-(4-bromotriazol-1-yl) cyclohexyl]-N-methyl-carbamate (1.20 g, 69% yield) as a white solid. LCMS (MM-ES+APCI, Pos): m/z 261.0 (M+H).

Step F. [1-[4-[tert-butoxycarbonyl (methyl) amino] cyclohexyl] triazol-4-yl] boronic acid. Synthesized according to Example 51, Step D, substituting tert-butyl (3S,4S)-3-fluoro-4-(4-iodo-5-methyl-pyrazol-1-yl)piperidine-1-carboxylate for tert-butyl N-[4-(4-bromotriazol-1-yl) cyclohexyl]-N-methyl-carbamate to give [1-[4-[tert-butoxycarbonyl (methyl) amino] cyclohexyl] triazol-4-yl] boronic acid (550 mg, crude) was obtained as a white solid, and used into the next step without further purification.

Step G. tert-butyl N-[4-[4-[3-cyano-4-[(3-fluoro-2-pyridyl)sulfanyl]pyrazolo[1,5-a]pyridin-6-yl]triazol-1-yl]cyclohexyl]-N-methyl-carbamate. Synthesized according to Example 51, Step E, substituting tert-butyl (3S,4S)-3-fluoro-4-[5-methyl-4-(4,4,5,5-tetramethyl-1,3,2-dioxaborolan-2-yl)pyrazol-1-yl]piperidine-1-carboxylate for [1-[4-[tert-butoxycarbonyl(methyl)amino]cyclohexyl]triazol-4-yl]boronic acid to give tert-butyl N-[4-[4-[3-cyano-4-[(3-fluoro-2-pyridyl)sulfanyl]pyrazolo[1,5-a]pyridin-6-yl]triazol-1-yl]cyclohexyl]-N-methyl-carbamate (23 mg, 2% yield) as a yellow solid. LCMS (MM-ES+APCI, Pos): m/z 549.2 (M+H).

Step H. 4-[(3-fluoro-2-pyridyl) sulfanyl]-6-[1-[4-(methylamino) cyclohexyl] triazol-4-yl]pyrazolo[1, 5-a]pyridine-3-carbonitrile. Synthesized according to Example 51, Step F, substituting tert-butyl (3S,4S)-4-[4-[3-cyano-4-[(3-fluoro-2-pyridyl)sulfanyl]pyrazolo[1,5-a]pyridin-6-yl]-5-methyl-pyrazol-1-yl]-3-fluoro-piperidine-1-carboxylate for tert-butyl N-[4-[4-[3-cyano-4-[(3-fluoro-2-pyridyl)sulfanyl]pyrazolo[1,5-a]pyridin-6-yl]triazol-1-yl]cyclohexyl]-N-methyl-carbamate to give crude 4-[(3-fluoro-2-pyridyl) sulfanyl]-6-[1-[4-(methylamino) cyclohexyl] triazol-4-yl] pyrazolo[1,5-a]pyridine-3-carbonitrile (26 mg, HCl) as a yellow solid and used into the next step without further purification. LCMS (MM-ES+APCI, Pos): m/z 449.2 (M+H).

Step I. 6-[1-[4-(dimethylamino) cyclohexyl] triazol-4-yl]-4-[(3-fluoro-2-pyridyl) sulfanyl]pyrazolo[1, 5-a]pyridine-3-carbonitrile. To a 8 mL vial equipped with a magnetic stir bar was added 4-[(3-fluoro-2-pyridyl)sulfanyl]-6-[1-[4-(methylamino)cyclohexyl]triazol-4-yl]pyrazolo[1,5-a]pyridine-3-carbonitrile (26.0 mg, 0.05 mmol), (HCHO)ₙ (8.62 mg, 0.27 mmol) and AcOH (0.003 mL, 0.05 mmol) followed by the addition of MeOH (0.5 mL). The mixture was stirred at 25° C. for 4 h. NaBH₃CN (16.8 mg, 0.27 mmol) was added into the mixture. The mixture was stirred at 25° C. for 12 h. The mixture was concentrated under reduced pressure and purified by preparative HPLC: (43%-73% acetonitrile/0.05% aqueous ammonia hydroxide) to give 6-[1-[4-(dimethylamino) cyclohexyl] triazol-4-yl]-4-[(3-fluoro-2-pyridyl) sulfanyl] pyrazolo[1,5-a]pyridine-3-carbonitrile (5 mg, 20% yield) as a white solid. LCMS (MM-ES+APCI, Pos): m/z 463.2 (M+H).

### Example 60

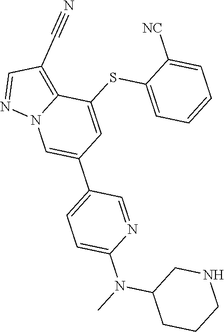 [SMILES: CN(c1ccc(-c2cc(Sc3ccccc3C#N)c3c(C#N)cnn3c2)cn1)C1CCCNC1]

### 4-(2-cyanophenyl)sulfanyl-6-[6-[methyl(3-piperidyl)amino]-3-pyridyl]pyrazolo[1,5-a]pyridine-3-carbonitrile (407)

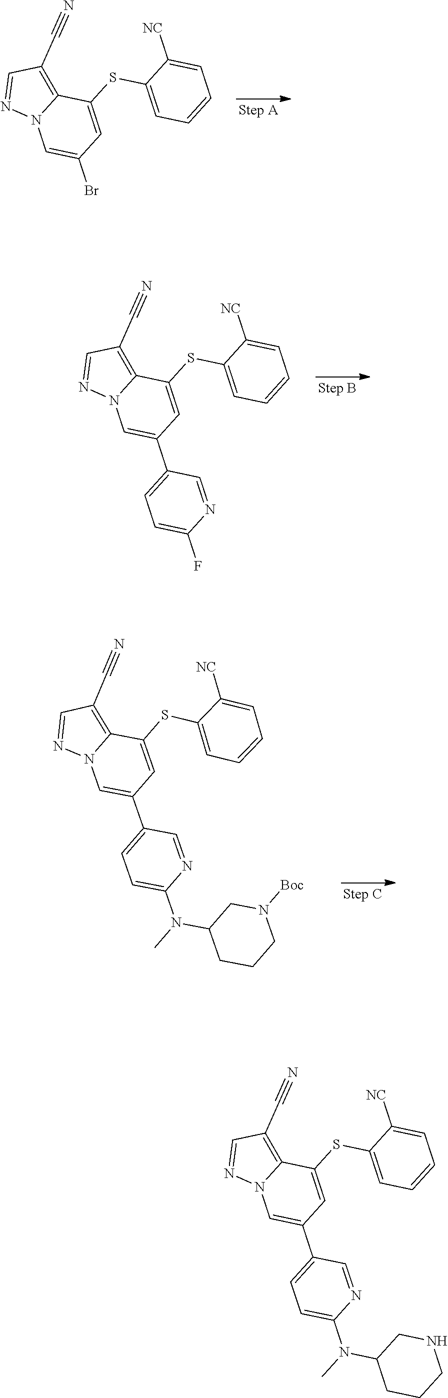 [SMILES: N#Cc1ccccc1Sc1cc(Br)cn2ncc(C#N)c12 ; N#Cc1ccccc1Sc1cc(-c2ccc(F)nc2)cn2ncc(C#N)c12 ; CN(c1ccc(-c2cc(Sc3ccccc3C#N)c3c(C#N)cnn3c2)cn1)C1CCCN(C(=O)OC(C)(C)C)C1 ; CN(c1ccc(-c2cc(Sc3ccccc3C#N)c3c(C#N)cnn3c2)cn1)C1CCCNC1]

Step A. 4-(2-cyanophenyl)sulfanyl-6-(6-fluoro-3-pyridyl)pyrazolo[1,5-a]pyridine-3-carbonitrile. Synthesized according to Example 51, Step E, substituting tert-butyl (3S,4S)-3-fluoro-4-[5-methyl-4-(4,4,5,5-tetramethyl-1,3,2-dioxaborolan-2-yl)pyrazol-1-yl]piperidine-1-carboxylate for 2-fluoro-5-(4,4,5,5-tetramethyl-1,3,2-dioxaborolan-2-yl)pyridine to give 4-(2-cyanophenyl)sulfanyl-6-(6-fluoro-3-pyridyl)pyrazolo[1,5-a]pyridine-3-carbonitrile (570 mg, 56% yield) as a white solid. LCMS (MM-ES+APCI, Pos): m/z 372.2 (M+H).

Step B. tert-butyl 3-[[5-[3-cyano-4-(2-cyanophenyl) sulfanyl-pyrazolo[1,5-a]pyridin-6-yl]-2-pyridyl]-methylamino] piperidine-1-carboxylate. To a 40 mL vial with a magnetic stir bar was added 4-(2-cyanophenyl)sulfanyl-6-(6-fluoro-3-pyridyl)pyrazolo[1,5-a]pyridine-3-carbonitrile (150 mg, 0.40 mmol) and tert-butyl 3-(methylamino)piperidine-1-carboxylate (173 mg, 0.8 mmol) followed by the addition of DMSO (2 mL). DIEA (0.14 mL, 0.8 mmol) was added into the mixture at 25° C. The mixture was heated to 120° C. and stirred for 48 h. Water (20 mL) was added to the reaction and the mixture was transferred to a separatory funnel. The mixture was extracted with ethyl acetate (10 mL×3). The combined organic layers were washed with brine (10 mL), dried over anhydrous sodium sulfate, filtered, concentrated and purified by silica gel column chromatography (0-10% petroleum ether/ethyl acetate) to give tert-butyl 3-[[5-[3-cyano-4-(2-cyanophenyl) sulfanyl-pyrazolo[1,5-a]pyridin-6-yl]-2-pyridyl]-methylamino] piperidine-1-carboxylate (80 mg, 35% yield) as a yellow solid.

Step C. 4-(2-cyanophenyl)sulfanyl-6-[6-[methyl(3-piperidyl)amino]-3-pyridyl]pyrazolo[1,5-a]pyridine-3-carbonitrile. Synthesized according to Example 51, Step F, substituting tert-butyl (3S,4S)-4-[4-[3-cyano-4-[(3-fluoro-2-pyridyl)sulfanyl]pyrazolo[1,5-a]pyridin-6-yl]-5-methyl-pyrazol-1-yl]-3-fluoro-piperidine-1-carboxylate for tert-butyl 3-[[5-[3-cyano-4-(2-cyanophenyl) sulfanyl-pyrazolo[1,5-a]pyridin-6-yl]-2-pyridyl]-methylamino]piperidine-1-carboxylate to give 4-(2-cyanophenyl)sulfanyl-6-[6-[methyl(3-piperidyl)amino]-3-pyridyl]pyrazolo[1,5-a]pyridine-3-carbonitrile (27.2 mg, 43% yield) as an off-white solid. LCMS (MM-ES+APCI, Pos): m/z 466.2 (M+H).

### Example 61

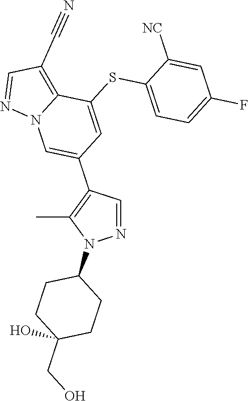 [SMILES: Cc1c(-c2cc(Sc3ccc(F)cc3C#N)c3c(C#N)cnn3c2)cnn1[C@H]1CC[C@@](O)(CO)CC1]

### 4-(2-cyano-4-fluoro-phenyl) sulfanyl-6-[1-[4-hydroxy-4-(hydroxymethyl) cyclohexyl]-5-methylpyrazol-4-yl] pyrazolo[1,5-a]pyridine-3-carbonitrile (438)

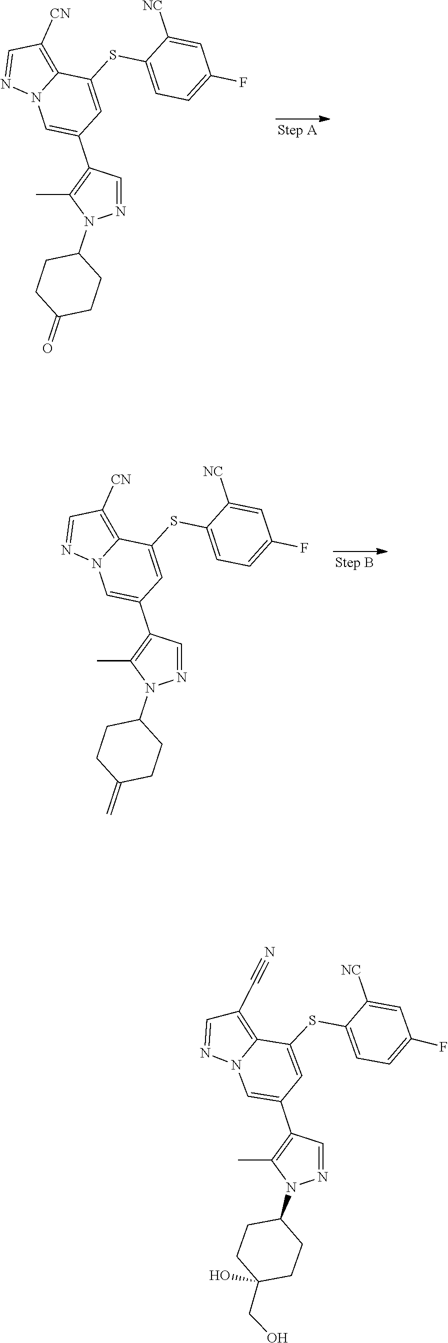 [SMILES: Cc1c(-c2cc(Sc3ccc(F)cc3C#N)c3c(C#N)cnn3c2)cnn1C1CCC(=O)CC1 ; C=C1CCC(n2ncc(-c3cc(Sc4ccc(F)cc4C#N)c4c(C#N)cnn4c3)c2C)CC1 ; Cc1c(-c2cc(Sc3ccc(F)cc3C#N)c3c(C#N)cnn3c2)cnn1[C@H]1CC[C@@](O)(CO)CC1]

Step A. 4-(2-cyano-4-fluoro-phenyl)sulfanyl-6-[5-methyl-1-(4-methylenecyclohexyl)pyrazol-4-yl]pyrazolo[1,5-a]pyridine-3-carbonitrile. To a 40 mL vial equipped with a magnetic stir bar was added methyl (triphenyl) phosphonium; bromide (350 mg, 0.98 mmol) followed by the addition of THF (5 mL). The solution was cooled to −70° C. LiHMDS (1 M, 0.97 mL, 1.96 mmol) was added dropwise at −70° C. The mixture was stirred at −70° C. for 1 h. 4-(2-cyano-4-fluoro-phenyl)sulfanyl-6-[5-methyl-1-(4-oxocyclohexyl)pyrazol-4-yl]pyrazolo[1,5-a]pyridine-3-carbonitrile (260 mg, 0.48 mmol, 1 eq) in THF (2 mL) was added dropwise. Water (2 mL) was added to the reaction slowly and the mixture was transferred to a separatory funnel. The mixture was extracted with ethyl acetate (2 mL×3). The combined organic layers were washed with brine (1 mL), dried over anhydrous sodium sulfate, filtered, concentrated and purified by silica gel column chromatography (30-50% petroleum ether/ethyl acetate) to give the desired product 4-(2-cyano-4-fluoro-phenyl)sulfanyl-6-[5-methyl-1-(4-methylenecyclohexyl)pyrazol-4-yl]pyrazolo[1,5-a]pyridine-3-carbonitrile (53 mg, 19% yield) as a yellow solid. LCMS (MM-ES+APCI, Pos): m/z 469.1 (M+H).

Step B. 4-(2-cyano-4-fluoro-phenyl) sulfanyl-6-[1-[4-hydroxy-4-(hydroxymethyl) cyclohexyl]-5-methylpyrazol-4-yl] pyrazolo[1,5-a]pyridine-3-carbonitrile. To an 8 mL vial equipped with a magnetic stir bar was added 4-(2-cyano-4-fluoro-phenyl)sulfanyl-6-[5-methyl-1-(4-methylenecyclohexyl)pyrazol-4-yl]pyrazolo[1,5-a]pyridine-3-carbonitrile (53 mg, 0.094 mmol) followed by the addition of acetone (1.5 mL) and H₂O (0.3 mL). NMO (0.0087 mL, 0.077 mmol) and K₂OsO₄·2H₂O (9 mg, 0.024 mmol) were added into the mixture at 25° C. The mixture was stirred at 25° C. for 24 h. The mixture was quenched by slow addition of H₂O (5 mL). The aqueous layer mixture was extracted with ethyl acetate (3 mL×3). The combined organic layers were washed with brine (3 mL), dried over anhydrous sodium sulfate, filtered, concentrated under reduced pressure and purified by preparative TLC (dichloromethane/methanol: 10/1) to give two crude products. Crude product 2 was re-purified by preparative HPLC (26%-56% acetonitrile/water (NH₄HCO₃)). After lyophilization, 4-(2-cyano-4-fluoro-phenyl) sulfanyl-6-[1-[4-hydroxy-4-(hydroxymethyl) cyclohexyl]-5-methylpyrazol-4-yl] pyrazolo[1,5-a]pyridine-3-carbonitrile (3 mg, 30% yield) was obtained as a white solid. LCMS (MM-ES+APCI, Pos): m/z 503.2 (M+H).

### Example 62

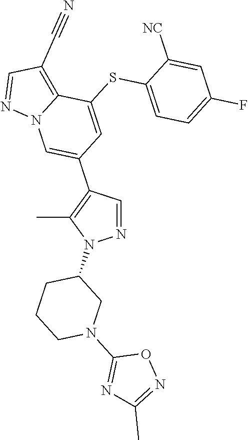 [SMILES: Cc1noc(N2CCC[C@H](n3ncc(-c4cc(Sc5ccc(F)cc5C#N)c5c(C#N)cnn5c4)c3C)C2)n1]

### 4-(2-cyano-4-fluoro-phenyl)sulfanyl-6-[5-methyl-1-[(3S)-1-(3-methyl-1,2,4-oxadiazol-5-yl)-3-piperidyl]pyrazol-4-yl]pyrazolo[1,5-a]pyridine-3-carbonitrile (455)

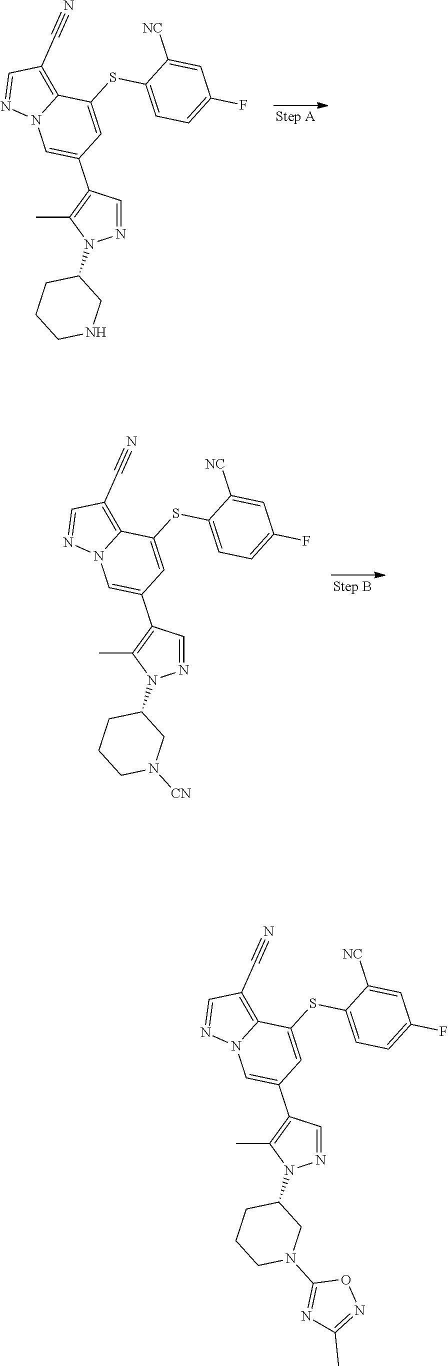 [SMILES: Cc1c(-c2cc(Sc3ccc(F)cc3C#N)c3c(C#N)cnn3c2)cnn1[C@H]1CCCNC1 ; Cc1c(-c2cc(Sc3ccc(F)cc3C#N)c3c(C#N)cnn3c2)cnn1[C@H]1CCCN(C#N)C1 ; Cc1noc(N2CCC[C@H](n3ncc(-c4cc(Sc5ccc(F)cc5C#N)c5c(C#N)cnn5c4)c3C)C2)n1]

Step A. 4-(2-cyano-4-fluoro-phenyl)sulfanyl-6-[1-[(3S)-1-cyano-3-piperidyl]-5-methyl-pyrazol-4-yl]pyrazolo[1,5-a]pyridine-3-carbonitrile. To a 40 mL vial equipped with a magnetic stir bar was added 4-(2-cyano-4-fluoro-phenyl)sulfanyl-6-[5-methyl-1-[(3S)-3-piperidyl]pyrazol-4-yl]pyrazolo[1,5-a]pyridine-3-carbonitrile (250 mg, 0.50 mmol) followed by the addition of ACN (2 mL). K₂CO₃ (279.78 mg, 2.02 mmol) was added. The solution was cooled to 0° C. and BrCN (0.12 mL, 1.60 mmol) in ACN (2 mL) was added dropwise. The mixture was allowed to warm to 25° C. and was stirred for 16 h. Aqueous saturated NaHCO₃ (15 mL) was added to the reaction slowly and the mixture was transferred to a separatory funnel. The mixture was extracted with ethyl acetate (10 mL×3). The combined organic layers were washed with brine (10 mL), dried over anhydrous sodium sulfate, filtered, concentrated and purified by silica gel column chromatography (0-10% petroleum ether/ethyl acetate) to give 4-(2-cyano-4-fluoro-phenyl)sulfanyl-6-[1-[(3S)-1-cyano-3-piperidyl]-5-methyl-pyrazol-4-yl]pyrazolo[1,5-a]pyridine-3-carbonitrile (140 mg, 57% yield) as a colorless gum.

Step B. 4-(2-cyano-4-fluoro-phenyl)sulfanyl-6-[5-methyl-1-[(3S)-1-(3-methyl-1,2,4-oxadiazol-5-yl)-3-piperidyl]pyrazol-4-yl]pyrazolo[1,5-a]pyridine-3-carbonitrile. To a 50 mL flask equipped with a magnetic stir bar was added 4-(2-cyano-4-fluoro-phenyl)sulfanyl-6-[1-[(3S)-1-cyano-3-piperidyl]-5-methyl-pyrazol-4-yl]pyrazolo[1,5-a]pyridine-3-carbonitrile (156 mg, 0.32 mmol) followed by the addition of EtOH (2 mL). N′-hydroxyacetamidine (28.74 mg, 0.39 mmol) was added followed by the addition of ZnCl₂ (1 M, 0.39 mL, 0.39 mmol) in EtOH (1 mL). The mixture was allowed to 25° C. and was stirred for 16 h. HCl (12 M, 0.08 mL) was added dropwise. The mixture was heated to 60° C. and stirred for 5 h. Water (10 mL) was added to the reaction and the mixture was transferred to a separatory funnel. The mixture was extracted with ethyl acetate (5 mL×3). The combined organic layers were washed with brine (5 mL), dried over anhydrous sodium sulfate, filtered, concentrated and purified by preparative HPLC (42%-72% acetonitrile/0.04% aqueous ammonia hydroxide+10 mM NH₄HCO₃). After lyophilization, 4-(2-cyano-4-fluoro-phenyl)sulfanyl-6-[5-methyl-1-[(3S)-1-(3-methyl-1,2,4-oxadiazol-5-yl)-3-piperidyl]pyrazol-4-yl]pyrazolo[1,5-a]pyridine-3-carbonitrile (22.05 mg, 12% yield) was obtained as a white solid. LCMS (MM-ES+APCI, Pos): m/z 540.3 (M+H).

### Example 63

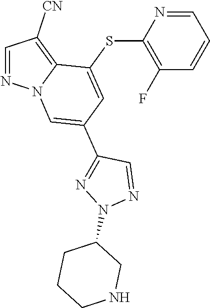 [SMILES: N#Cc1cnn2cc(-c3cnn([C@H]4CCCNC4)n3)cc(Sc3ncccc3F)c12]

### 4-[(3-fluoro-2-pyridyl)sulfanyl]-6-[2-[(3S)-3-piperidyl]triazol-4-yl]pyrazolo[1,5-a]pyridine-3-carbonitrile (457)

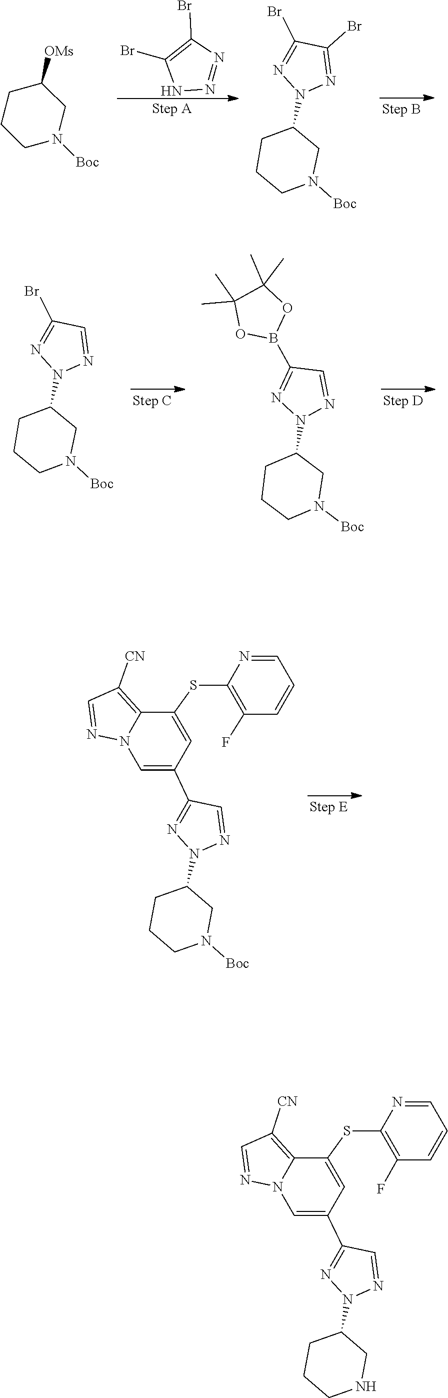

Step A. tert-butyl (3S)-3-(4,5-dibromotriazol-2-yl)piperidine-1-carboxylate. Synthesized according to Example 51, Step B, substituting tert-butyl (3S,4R)-3-fluoro-4-methylsulfonyloxy-piperidine-1-carboxylate for tert-butyl (3R)-3-methylsulfonyloxypiperidine-1-carboxylate and 4-iodo-1H-pyrazole for 4,5-dibromo-1H-triazole to give tert-butyl (3S)-3-(4,5-dibromotriazol-2-yl)piperidine-1-carboxylate (2.2 g, 38% yield) as a yellow solid. LCMS (MM-ES+APCI, Pos): m/z 354.8 (M+H).

Step B. tert-butyl (3S)-3-(4-bromotriazol-2-yl)piperidine-1-carboxylate. To a 40 mL vial equipped with a magnetic stir bar was added tert-butyl (3S)-3-(4,5-dibromotriazol-2-yl)piperidine-1-carboxylate (1.5 g, 3.23 mmol) followed by the addition of THF (15 mL). The vial was then blown with nitrogen for 2 min. i-PrMgCl (2 M, 3.23 mL, 6.46 mmol) was added into the mixture at −10° C. The mixture was stirred at −10° C. for 1 h. Water (30 mL) was added to the reaction and the mixture was transferred to a separatory funnel. The mixture was extracted with ethyl acetate (30 mL×3). The combined organic layers were washed with brine (30 mL), dried over anhydrous sodium sulfate, filtered, concentrated and purified by silica gel column chromatography (0-10% petroleum ether/ethyl acetate) to give tert-butyl (3S)-3-(4-bromotriazol-2-yl)piperidine-1-carboxylate (900 mg, 82% yield) as a white solid. LCMS (MM-ES+APCI, Pos): m/z 231.1 (M+H).

Step C. tert-butyl (3S)-3-[4-(4,4,5,5-tetramethyl-1,3,2-dioxaborolan-2-yl)triazol-2-yl]piperidine-1-carboxylate. Synthesized according to Example 51, Step D, substituting tert-butyl (3S,4S)-3-fluoro-4-(4-iodo-5-methyl-pyrazol-1-yl)piperidine-1-carboxylate for tert-butyl (3S)-3-(4-bromotriazol-2-yl)piperidine-1-carboxylate to give tert-butyl (3S)-3-[4-(4,4,5,5-tetramethyl-1,3,2-dioxaborolan-2-yl)triazol-2-yl]piperidine-1-carboxylate (400 mg, 33% yield) was obtained as a yellow solid. LCMS (MM-ES+APCI, Pos): m/z 241.0 (M+H).

Step D. tert-butyl (3S)-3-[4-[3-cyano-4-[(3-fluoro-2-pyridyl)sulfanyl]pyrazolo[1,5-a]pyridin-6-yl]triazol-2-yl]piperidine-1-carboxylate. Synthesized according to Example 51, Step E, substituting tert-butyl (3S,4S)-3-fluoro-4-[5-methyl-4-(4,4,5,5-tetramethyl-1,3,2-dioxaborolan-2-yl)pyrazol-1-yl]piperidine-1-carboxylate for 6-bromo-4-[(3-fluoro-2-pyridyl)sulfanyl]pyrazolo[1,5-a]pyridine-3-carbonitrile to give tert-butyl (3S)-3-[4-[3-cyano-4-[(3-fluoro-2-pyridyl)sulfanyl]pyrazolo[1,5-a]pyridin-6-yl]triazol-2-yl]piperidine-1-carboxylate (60 mg, 48% yield) as a black brown oil. LCMS (MM-ES+APCI, Pos): m/z 421.1 (M+H).

Step E. 4-[(3-fluoro-2-pyridyl)sulfanyl]-6-[2-[(3S)-3-piperidyl]triazol-4-yl]pyrazolo[1,5-a]pyridine-3-carbonitrile. Synthesized according to Example 51, Step F, substituting tert-butyl (3S,4S)-4-[4-[3-cyano-4-[(3-fluoro-2-pyridyl)sulfanyl]pyrazolo[1,5-a]pyridin-6-yl]-5-methyl-pyrazol-1-yl]-3-fluoro-piperidine-1-carboxylate for tert-butyl (3S)-3-[4-[3-cyano-4-[(3-fluoro-2-pyridyl)sulfanyl]pyrazolo[1,5-a]pyridin-6-yl]triazol-2-yl]piperidine-1-carboxylate to give 4-[(3-fluoro-2-pyridyl)sulfanyl]-6-[2-[(3S)-3-piperidyl]triazol-4-yl]pyrazolo[1,5-a]pyridine-3-carbonitrile (4.9 mg, 18% yield) was obtained as a white solid. LCMS (MM-ES+APCI, Pos): m/z 421.3 (M+H).

### Example 64

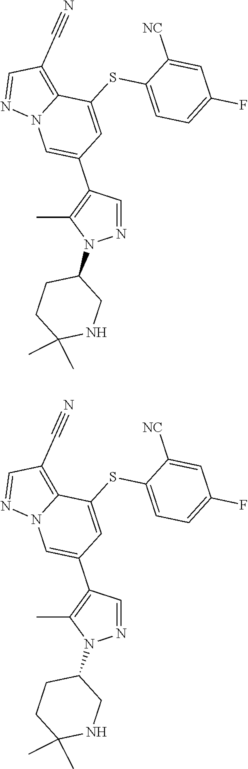

### 4-(2-cyano-4-fluoro-phenyl)sulfanyl-6-[1-[(3R)-6,6-dimethyl-3-piperidyl]-5-methyl-pyrazol-4-yl]pyrazolo[1,5-a]pyridine-3-carbonitrile (528), 4-(2-cyano-4-fluoro-phenyl)sulfanyl-6-[1-[(3R)-6,6-dimethyl-3-piperidyl]-5-methyl-pyrazol-4-yl]pyrazolo[1,5-a]pyridine-3-carbonitrile (529)

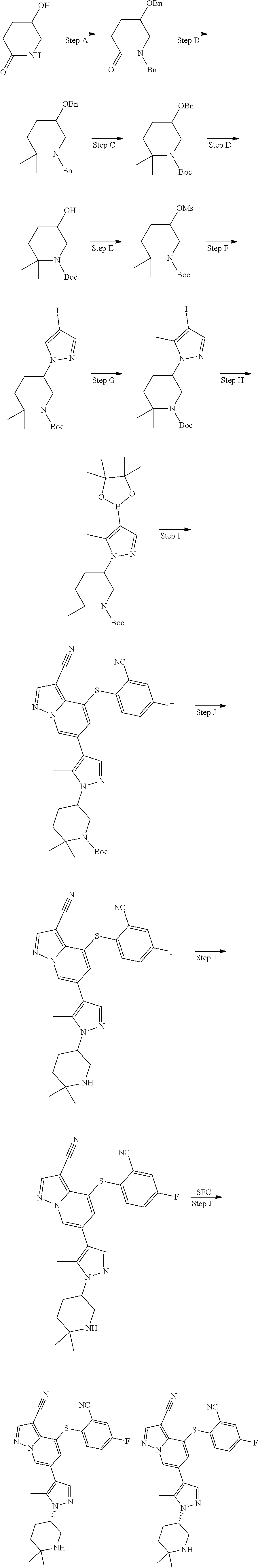

Step A. 1-benzyl-5-benzyloxy-piperidin-2-one. To a 100 mL round-bottom flask equipped with a magnetic stir bar was added 5-hydroxypiperidin-2-one (2.5 g, 21.71 mmol) followed by the addition of DMSO (25 mL). KOH (3.65 g, 65.14 mmol) and BnBr (7.74 mL, 65.14 mmol) was added into the mixture at 25° C. The mixture was stirred at 25° C. for 4 h. Water (200 mL) was added to the reaction and the mixture was transferred to a separatory funnel. The mixture was extracted with ethyl acetate (100 mL×3). The combined organic layers were washed with brine (100 mL), dried over anhydrous sodium sulfate, filtered, concentrated and purified by silica gel column chromatography (25-50% ethyl acetate in petroleum ether) to give the desired product 1-benzyl-5-benzyloxy-piperidin-2-one (1.6 g, 23% yield) obtained as a gray solid. LCMS (MM-ES+APCI, Pos): m/z 296.3 (M+H).

Step B. 1-benzyl-5-benzyloxy-2,2-dimethyl-piperidine. To a solution of 1-benzyl-5-benzyloxy-piperidin-2-one (3.2 g, 9.93 mmol) and DTBMP (2.45 g, 11.92 mmol) in DCM (180 mL) at −78° C. was added Tf₂O (1.97 mL, 11.92 mmol) under N₂. The mixture was stirred at −78° C. for 1 h. A solution of MeMgBr (3 M, 9.93 mL, 9.93 mmol) was added dropwise. The reaction mixture was warmed to 20° C. and stirred for another 2 h. Aqueous NH₄Cl (20 mL) was added, followed by slow addition of water (200 mL) and transferred to a separatory funnel. The mixture was extracted with DCM (50 mL×3). The combined organic layers were washed with brine (100 mL), dried over anhydrous sodium sulfate, filtered, concentrated and purified by silica gel column chromatography (0-10% ethyl acetate in petroleum ether) to give the desired product 1-benzyl-5-benzyloxy-2,2-dimethyl-piperidine (2.6 g, 84% yield) as a colorless oil. LCMS (MM-ES+APCI, Pos): m/z 310.2 (M+H).

Step C. tert-butyl 5-benzyloxy-2,2-dimethyl-piperidine-1-carboxylate. A mixture of 1-benzyl-5-benzyloxy-2,2-dimethyl-piperidine (2.6 g, 8.36 mmol), Boc₂O (3.85 mL, 16.74 mmol) and Pd(OH)₂ (800 mg, 8.36 mmol, 20% purity) in MeOH (50 mL) was hydrogenated at 20° C. under H₂ (50 psi) for 16 h. The reaction solution was filtered, the filter cake was washed with MeOH (15 mL×3) and the filtrate was concentrated to afford the product tert-butyl 5-benzyloxy-2,2-dimethyl-piperidine-1-carboxylate (3.3 g, crude) obtained as a colorless oil and used into the next step without further purification.

Step D. tert-butyl 5-hydroxy-2,2-dimethyl-piperidine-1-carboxylate. A mixture of tert-butyl 5-benzyloxy-2,2-dimethyl-piperidine-1-carboxylate (3 g, 9.39 mmol) and Pd(OH)₂ (500 mg, 0.71 mmol, 20% purity) in MeOH (50 mL) was hydrogenated at 60° C. under H₂ (50 psi) for 6 h. The reaction solution was filtered, the filter cake washed with MeOH (15 mL×3) and the filtrate was concentrated to afford the product tert-butyl 5-hydroxy-2,2-dimethyl-piperidine-1-carboxylate (1.7 g, 72% yield) as a colorless solid.

Step E. tert-butyl 2,2-dimethyl-5-methylsulfonyloxy-piperidine-1-carboxylate. Synthesized according to Example 50, Step A, substituting 4-[(3-fluoro-2-pyridyl)sulfanyl]-6-[5-methyl-1-(4-piperidyl)pyrazol-4-yl]pyrazolo[1,5-a]pyridine-3-carbonitrile for tert-butyl 5-hydroxy-2,2-dimethyl-piperidine-1-carboxylate to give tert-butyl 2,2-dimethyl-5-methylsulfonyloxy-piperidine-1-carboxylate (620 mg, 92% yield) obtained as a brown oil.

Step F. tert-butyl 5-(4-iodopyrazol-1-yl)-2,2-dimethyl-piperidine-1-carboxylate. Synthesized according to Example 51, Step B, substituting tert-butyl (3S,4R)-3-fluoro-4-methylsulfonyloxy-piperidine-1-carboxylate for tert-butyl 2,2-dimethyl-5-methylsulfonyloxy-piperidine 1-carboxylate to give tert-butyl 5-(4-iodopyrazol-1-yl)-2,2-dimethyl-piperidine-1-carboxylate (430 mg, 49% yield) as a white solid. LCMS (MM-ES+APCI, Pos): m/z 405.9 (M+H).

Step G. tert-butyl 5-(4-iodo-5-methyl-pyrazol-1-yl)-2,2-dimethyl-piperidine-1-carboxylate. Synthesized according to Example 51, Step C, substituting tert-butyl (3S,4S)-3-fluoro-4-(4-iodopyrazol-1-yl)piperidine-1-carboxylate for tert-butyl 5-(4-iodopyrazol-1-yl)-2,2-dimethyl-piperidine-1-carboxylate to give tert-butyl 5-(4-iodo-5-methyl-pyrazol-1-yl)-2,2-dimethyl-piperidine-1-carboxylate (445 mg, 79% yield) as a white solid. LCMS (MM-ES+APCI, Pos): m/z 420.1 (M+H).

Step H. tert-butyl 2,2-dimethyl-5-[5-methyl-4-(4,4,5,5-tetramethyl-1,3,2-dioxaborolan-2-yl)pyrazol-1-yl]piperidine-1-carboxylate. Synthesized according to Example 51, Step D, substituting tert-butyl (3S,4S)-3-fluoro-4-(4-iodo-5-methyl-pyrazol-1-yl)piperidine-1-carboxylate for tert-butyl 5-(4-iodo-5-methyl-pyrazol-1-yl)-2,2-dimethyl-piperidine-1-carboxylate to give tert-butyl 2,2-dimethyl-5-[5-methyl-4-(4,4,5,5-tetramethyl-1,3,2-dioxaborolan-2-yl)pyrazol-1-yl]piperidine-1-carboxylate (249 mg, 46% yield) as a white solid. LCMS (MM-ES+APCI, Pos): m/z 420.2 (M+H).

Step I. tert-butyl 5-[4-[3-cyano-4-(2-cyano-4-fluoro-phenyl)sulfanyl-pyrazolo[1,5-a]pyridin-6-yl]-5-methylpyrazol-1-yl]-2,2-dimethyl-piperidine-1-carboxylate. Synthesized according to Example 51, Step E, substituting tert-butyl (3S,4S)-3-fluoro-4-[5-methyl-4-(4,4,5,5-tetramethyl-1,3,2-dioxaborolan-2-yl)pyrazol-1-yl]piperidine-1-carboxylate for tert-butyl 2,2-dimethyl-5-[5-methyl-4-(4,4,5,5-tetramethyl-1,3,2-dioxaborolan-2-yl)pyrazol-1-yl]piperidine-1-carboxylate to give tert-butyl 5-[4-[3-cyano-4-(2-cyano-4-fluoro-phenyl)sulfanyl-pyrazolo[1,5-a]pyridin-6-yl]-5-methylpyrazol-1-yl]-2,2-dimethyl-piperidine-1-carboxylate (130 mg, 44% yield) as a brown solid. LCMS (MM-ES+APCI, Pos): m/z 586.4 (M+H).

Step J. 4-(2-cyano-4-fluoro-phenyl)sulfanyl-6-[1-(6,6-dimethyl-3-piperidyl)-5-methyl-pyrazol-4-yl]pyrazolo[1,5-a]pyridine-3-carbonitrile. Synthesized according to Example 51, Step F, substituting tert-butyl (3S,4S)-4-[4-[3-cyano-4-[(3-fluoro-2-pyridyl)sulfanyl]pyrazolo[1,5-a]pyridin-6-yl]-5-methyl-pyrazol-1-yl]-3-fluoro-piperidine-1-carboxylate for tert-butyl 5-[4-[3-cyano-4-(2-cyano-4-fluoro-phenyl)sulfanyl-pyrazolo[1,5-a]pyridin-6-yl]-5-methyl-pyrazol-1-yl]-2,2-dimethyl-piperidine-1-carboxylate to give 4-(2-cyano-4-fluoro-phenyl)sulfanyl-6-[1-(6,6-dimethyl-3-piperidyl)-5-methyl-pyrazol-4-yl]pyrazolo[1,5-a]pyridine-3-carbonitrile (70 mg, 86% yield) as a white solid. LCMS (MM-ES+APCI, Pos): m/z 486.2 (M+H).

### Example 65

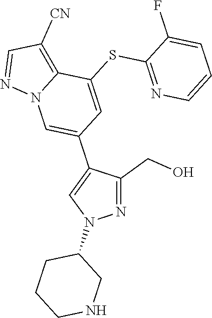 [SMILES: N#Cc1cnn2cc(-c3cn([C@H]4CCCNC4)nc3CO)cc(Sc3ncccc3F)c12]

### 4-[(3-fluoro-2-pyridyl)sulfanyl]-6-[3-(hydroxymethyl)-1-[(3S)-3-piperidyl]pyrazol-4-yl]pyrazolo[1,5-a]pyridine-3-carbonitrile (530)

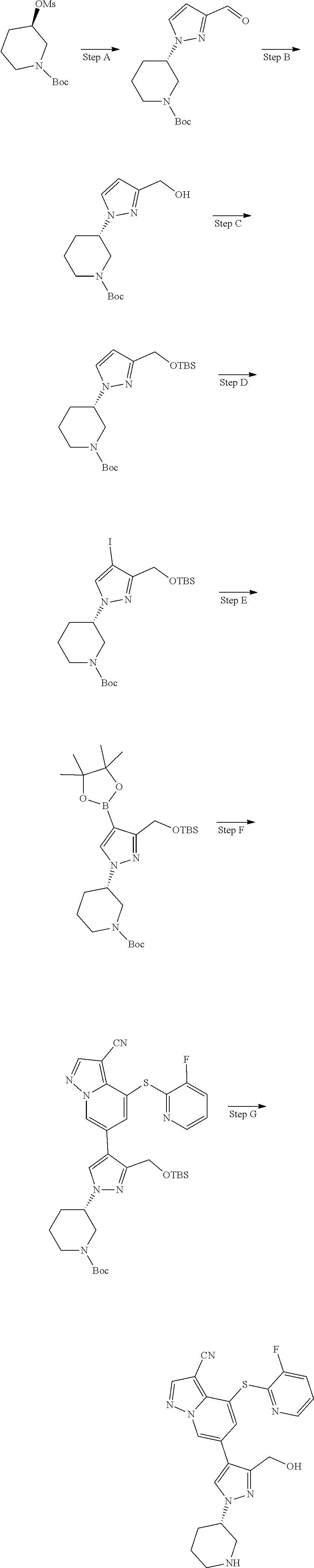

Step A. tert-butyl (3S)-3-(3-formylpyrazol-1-yl)piperidine-1-carboxylate. Synthesized according to Example 51, Step B, substituting tert-butyl (3S,4R)-3-fluoro-4-methylsulfonyloxy-piperidine-1-carboxylate for tert-butyl (3R)-3-methylsulfonyloxypiperidine-1-carboxylate and 4-iodo-1H-pyrazole for 1H-pyrazole-3-carbaldehyde to give tert-butyl (3S)-3-(3-formylpyrazol-1-yl)piperidine-1-carboxylate (3.5 g, 31% yield) as a colorless oil.

Step B. tert-butyl (3S)-3-[3-(hydroxymethyl)pyrazol-1-yl]piperidine-1-carboxylate. To a 100 mL round-bottom flask equipped with a magnetic stir bar was added tert-butyl (3S)-3-(3-formylpyrazol-1-yl)piperidine-1-carboxylate (3.3 g, 11.81 mmol) followed by the addition of MeOH (30 mL). The solution was cooled to 0° C. NaBH₄ (1.33 g, 35.16 mmol) was added in portions. The mixture was allowed to warm to 20° C. and was stirred for 2 h. Saturated aqueous ammonium chloride (30 mL) was added to the reaction and the mixture was transferred to a separatory funnel. The mixture was extracted with ethyl acetate (50 mL×3). The combined organic layers were washed with brine (50 mL), dried over anhydrous sodium sulfate, filtered, concentrated and purified by silica gel column chromatography (20-60% petroleum ether/ethyl acetate) to give the desired product tert-butyl (3S)-3-[3-(hydroxymethyl)pyrazol-1-yl]piperidine-1-carboxylate (2.1 g, 63% yield) was obtained as a colorless oil.

Step C. tert-butyl (3S)-3-[3-[[tert-butyl(dimethyl)silyl]oxymethyl]pyrazol-1-yl]piperidine-1-carboxylate. To a 100 mL round-bottom flask equipped with a magnetic stir bar were added tert-butyl (3S)-3-[3-(hydroxymethyl)pyrazol-1-yl]piperidine-1-carboxylate (1.3 g, 4.62 mmol) and imidazole (787 mg, 11.56 mmol) followed by the addition of THF (15 mL). Tert-butyl-chloro-dimethyl-silane (1.14 mL 9.24 mmol) followed by the addition of THF (15 mL) was slowly added into the mixture at 0° C. The mixture was stirred at 25° C. for 1 h. Water (15 mL) was added to the reaction and the mixture was transferred to a separatory funnel. The mixture was extracted with ethyl acetate (15 mL×3). The combined organic layers were washed with brine (30 mL), dried over anhydrous sodium sulfate, filtered, and concentrated under reduced pressure to afford the crude product tert-butyl (3S)-3-[3-[[tert-butyl(dimethyl)silyl]oxymethyl]pyrazol-1-yl]piperidine-1-carboxylate (1.75 g, 96% yield) as a yellow oil.

Step D. tert-butyl (3S)-3-[3-[[tert-butyl(dimethyl)silyl]oxymethyl]-4-iodo-pyrazol-1-yl]piperidine-1-carboxylate. To an 8 mL vial equipped with a magnetic stir bar was added tert-butyl (3S)-3-[3-[[tertbutyl(dimethyl)silyl]oxymethyl]pyrazol-1-yl]piperidine-1-carboxylate (300 mg, 0.76 mmol) followed by the addition of ACN (3 mL). NIS (511 mg, 2.27 mmol) was added into the mixture at 25° C. The mixture was heated to 40° C. and stirred for 1 h. Water (3 mL) was added to the reaction and the mixture was transferred to a separatory funnel. The mixture was extracted with ethyl acetate (3 mL×3). The combined organic layers were washed with brine (9 mL), dried over anhydrous sodium sulfate, filtered, concentrated and purified by silica gel column chromatography (0-80% petroleum ether/ethyl acetate) to give the desired product tert-butyl (3S)-3-[3-[[tert-butyl(dimethyl)silyl]oxymethyl]-4-iodo-pyrazol-1-yl]piperidine-1-carboxylate (330 mg, 80% yield) as a colorless oil. LCMS (MM-ES+APCI, Pos): m/z 522.3 (M+H).

Step E. tert-butyl (3S)-3-[3-[[tert-butyl(dimethyl)silyl]oxymethyl]-4-(4,4,5,5-tetramethyl-1,3,2-dioxaborolan-2-yl)pyrazol-1-yl]piperidine-1-carboxylate. Synthesized according to Example 51, Step D, substituting tert-butyl (3S,4S)-3-fluoro-4-(4-iodo-5-methyl-pyrazol-1-yl)piperidine-1-carboxylate for tert-butyl (3S)-3-[3-[[tert-butyl(dimethyl)silyl]oxymethyl]-4-iodo-pyrazol-1-yl]piperidine-1-carboxylate to give tert-butyl (3S)-3-[3-[[tert-butyl(dimethyl)silyl]oxymethyl]-4-(4,4,5,5-tetramethyl-1,3,2-dioxaborolan-2-yl)pyrazol-1-yl]piperidine-1-carboxylate (500 mg, 58% yield) as a colorless oil. LCMS (MM-ES+APCI, Pos): m/z 522.5 (M+H).

Step F. tert-butyl (3S)-3-[3-[[tert-butyl(dimethyl)silyl]oxymethyl]-4-[3-cyano-4-[(3-fluoro-2-pyridyl)sulfanyl]pyrazolo[1,5-a]pyridin-6-yl]pyrazol-1-yl]piperidine-1-carboxylate. Synthesized according to Example 51, Step E, substituting tert-butyl (3S,4S)-3-fluoro-4-[5-methyl-4-(4,4,5,5-tetramethyl-1,3,2-dioxaborolan-2-yl)pyrazol-1-yl]piperidine-1-carboxylate for tert-butyl (3S)-3-[3-[[tert-butyl(dimethyl)silyl]oxymethyl]-4-(4,4,5,5-tetramethyl-1,3,2-dioxaborolan-2-yl)pyrazol-1-yl]piperidine-1-carboxylate to give tert-butyl (3S)-3-[3-[[tert-butyl(dimethyl)silyl]oxymethyl]-4-[3-cyano-4-[(3-fluoro-2-pyridyl)sulfanyl]pyrazolo[1,5-a]pyridin-6-yl]pyrazol-1-yl]piperidine-1-carboxylate (270 mg, 86% yield) as a yellow gum. LCMS (MM-ES+APCI, Pos): m/z 664.5 (M+H).

Step G. 4-[(3-fluoro-2-pyridyl)sulfanyl]-6-[3-(hydroxymethyl)-1-[(3S)-3-piperidyl]pyrazol-4-yl]pyrazolo[1,5-a]pyridine-3-carbonitrile. Synthesized according to Example 51, Step F, substituting tert-butyl (3S,4S)-4-[4-[3-cyano-4-[(3-fluoro-2-pyridyl)sulfanyl]pyrazolo[1,5-a]pyridin-6-yl]-5-methyl-pyrazol-1-yl]-3-fluoro-piperidine-1-carboxylate for tert-butyl (3S)-3-[3-[[tert-butyl(dimethyl)silyl]oxymethyl]-4-[3-cyano-4-[(3-fluoro-2-pyridyl)sulfanyl]pyrazolo[1,5-a]pyridin-6-yl]pyrazol-1-yl]piperidine-1-carboxylate to give 4-[(3-fluoro-2-pyridyl)sulfanyl]-6-[3-(hydroxymethyl)-1-[(3S)-3-piperidyl]pyrazol-4-yl]pyrazolo[1,5-a]pyridine-3-carbonitrile (153 mg, 97% yield) as a white solid. LCMS (MM-ES+APCI, Pos): m/z 450.3 (M+H).

### Example 66

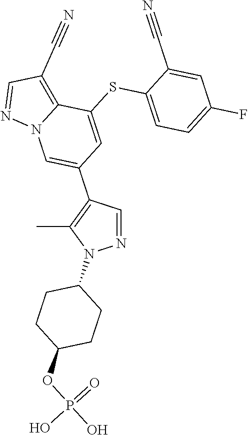 [SMILES: Cc1c(-c2cc(Sc3ccc(F)cc3C#N)c3c(C#N)cnn3c2)cnn1[C@H]1CC[C@H](OP(=O)(O)O)CC1]

### [4-[4-[3-cyano-4-(2-cyano-4-fluoro-phenyl)sulfanyl-pyrazolo[1,5-a]pyridin-6-yl]-5-methylpyrazol-1-yl]cyclohexyl] dihydrogen phosphate (543)

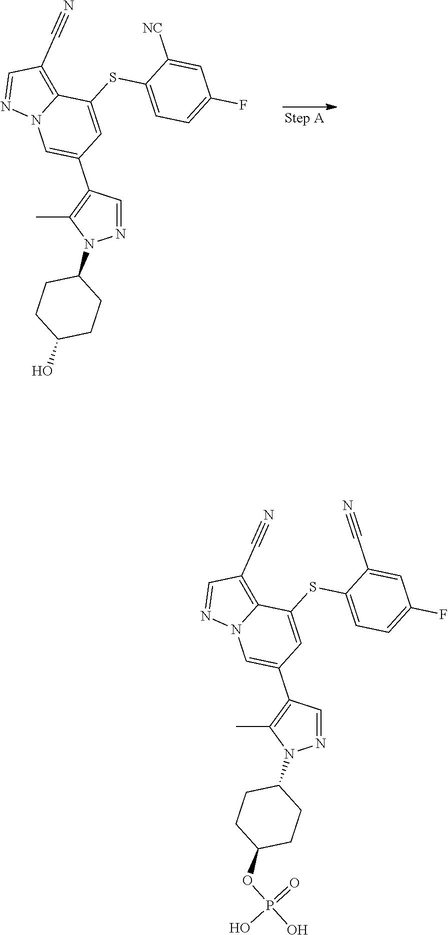 [SMILES: Cc1c(-c2cc(Sc3ccc(F)cc3C#N)c3c(C#N)cnn3c2)cnn1[C@H]1CC[C@H](O)CC1 ; Cc1c(-c2cc(Sc3ccc(F)cc3C#N)c3c(C#N)cnn3c2)cnn1[C@H]1CC[C@H](OP(=O)(O)O)CC1]

Step A. [4-[4-[3-cyano-4-(2-cyano-4-fluoro-phenyl)sulfanyl-pyrazolo[1,5-a]pyridin-6-yl]-5-methylpyrazol-1-yl]cyclohexyl] dihydrogen phosphate. To a 40 mL vial with a magnetic stir bar was added 4-(2-cyano-4-fluoro-phenyl) sulfanyl-6-[1-(4-hydroxycyclohexyl)-5-methyl-pyrazol-4-yl] pyrazolo[1,5-a]pyridine-3-carbonitrile (120 mg, 0.23 mmol) followed by the addition of THF (2 mL). Pyridine (0.038 mL, 0.47 mmol) was added into the mixture at 25° C. The mixture was cooled to 0° C. POCI3 (0.03 mL, 0.28 mmol) was added dropwise. The mixture was stirred at 0° C. for 0.5 h. Water (20 mL) was added to the reaction and the mixture was transferred to a separatory funnel. The mixture was extracted with ethyl acetate (10 mL×3). The combined organic layers were washed with brine (10 mL), dried over anhydrous sodium sulfate, filtered, concentrated and purified by preparative HPLC (10%-40% acetonitrile/0.04% aqueous ammonia hydroxide+10 mM NH₄HCO₃. After lyophilization, [4-[4-[3-cyano-4-(2-cyano-4-fluoro-phenyl)sulfanyl-pyrazolo[1,5-a]pyridin-6-yl]-5-methylpyrazol-1-yl]cyclohexyl] dihydrogen phosphate (31.7 mg, 24% yield) was obtained as a white solid. LCMS (MM-ES+APCI, Pos): m/z 533.1 (M+H).

### Example 67

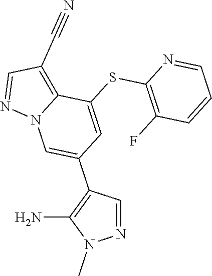 [SMILES: Cn1ncc(-c2cc(Sc3ncccc3F)c3c(C#N)cnn3c2)c1N]

### 6-(5-amino-1-methyl-pyrazol-4-yl)-4-[(3-fluoro-2-pyridyl)sulfanyl]pyrazolo[1,5-a]pyridine-3-carbonitrile (545)

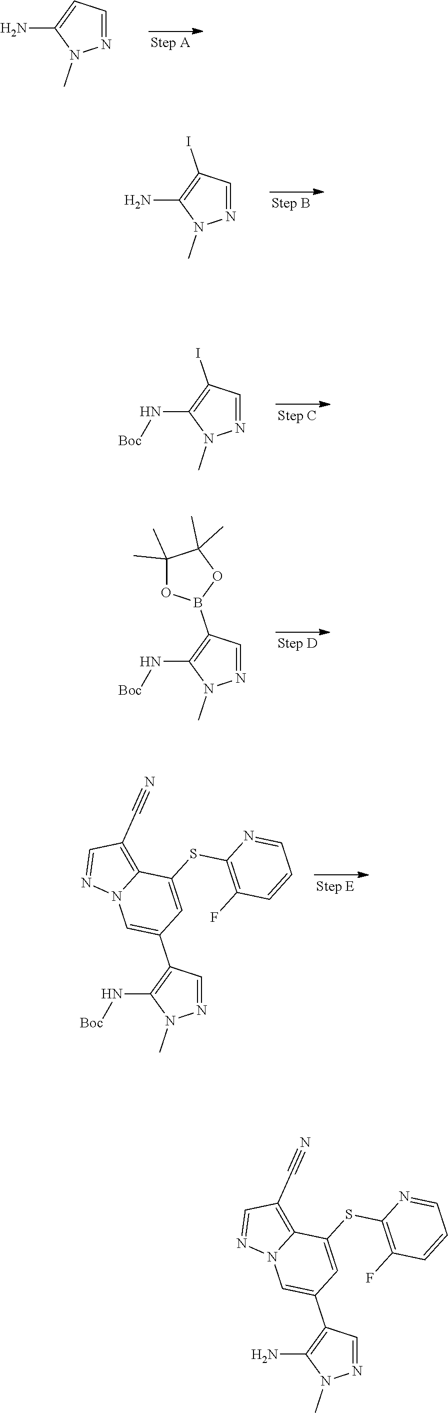

Step A. 4-iodo-2-methyl-pyrazol-3-amine. To a 40 mL vial equipped with a magnetic stir bar was added 2-methylpyrazol-3-amine (1 g, 10.30 mmol) followed by the addition of DCM (12 mL). NIS (2.78 g, 12.36 mmol) was added into the mixture at 25° C. The mixture was stirred at 25° C. for 1 h. Water (30 mL) was added to the reaction and the mixture was transferred to a separatory funnel. The mixture was extracted with ethyl acetate (30 mL×3). The combined organic layers were washed with brine (20 mL), dried over anhydrous sodium sulfate, filtered, concentrated and purified by silica gel column chromatography (0-30% petroleum ether/ethyl acetate) to give the desired product 4-iodo-2-methyl-pyrazol-3-amine (2 g, 84% yield) as a red solid. LCMS (MM-ES+APCI, Pos): m/z 233.8 (M+H).

Step B. tert-butyl N-(4-iodo-2-methyl-pyrazol-3-yl) carbamate. To a 10 mL round-bottom flask equipped with a magnetic stir bar was added 4-iodo-2-methyl-pyrazol-3-amine (1 g, 4.34 mmol), DMAP (53 mg, 0.43 mmol) and TEA (1.21 mL, 8.68 mmol) followed by the addition of DCM (10 mL). Boc₂O (1.04 g, 4.77 mmol) in DCM (3 mL) was added dropwise. The mixture was allowed to 25° C. and stir for 12 h. Water (20 mL) was added to the reaction and the mixture was transferred to a separatory funnel. The mixture was extracted with ethyl acetate (20 mL×3). The combined organic layers were washed with brine (5 mL), dried over anhydrous sodium sulfate, filtered, concentrated and purified by silica gel column chromatography (0-30% petroleum ether/ethyl acetate) to give tert-butyl N-(4-iodo-2-methyl-pyrazol-3-yl) carbamate (500 mg, 35% yield) as a white solid. LCMS (MM-ES+APCI, Pos): m/z 324.1 (M+H).

Step C. tert-butyl N-[2-methyl-4-(4,4,5,5-tetramethyl-1,3,2-dioxaborolan-2-yl)pyrazol-3-yl]carbamate. Synthesized according to Example 51, Step D, substituting tert-butyl (3S,4S)-3-fluoro-4-(4-iodo-5-methyl-pyrazol-1-yl)piperidine-1-carboxylate for tert-butyl N-(4-iodo-2-methyl-pyrazol-3-yl) carbamate to give tert-butyl N-[2-methyl-4-(4,4,5,5-tetramethyl-1,3,2-dioxaborolan-2-yl)pyrazol-3-yl]carbamate (165 mg, 78% yield) as a white solid. LCMS (MM-ES+APCI, Pos): m/z 324.3 (M+H).

Step D. tert-butyl N-[4-[3-cyano-4-[(3-fluoro-2-pyridyl)sulfanyl]pyrazolo[1,5-a]pyridin-6-yl]-2-methyl-pyrazol-3-yl]carbamate. Synthesized according to Example 51, Step E, substituting tert-butyl (3S,4S)-3-fluoro-4-[5-methyl-4-(4,4,5,5-tetramethyl-1,3,2-dioxaborolan-2-yl)pyrazol-1-yl]piperidine-1-carboxylate for tert-butyl N-[2-methyl-4-(4,4,5,5-tetramethyl-1,3,2-dioxaborolan-2-yl)pyrazol-3-yl]carbamate to give tert-butyl N-[4-[3-cyano-4-[(3-fluoro-2-pyridyl)sulfanyl]pyrazolo[1,5-a]pyridin-6-yl]-2-methyl-pyrazol-3-yl]carbamate (100 mg, 63% yield) as a white solid. LCMS (MM-ES+APCI, Pos): m/z 466.0 (M+H).

Step E. 6-(5-amino-1-methyl-pyrazol-4-yl)-4-[(3-fluoro-2-pyridyl)sulfanyl]pyrazolo[1,5-a]pyridine-3-carbonitrile. Synthesized according to Example 51, Step F, substituting tert-butyl (3S,4S)-4-[4-[3-cyano-4-[(3-fluoro-2-pyridyl)sulfanyl]pyrazolo[1,5-a]pyridin-6-yl]-5-methyl-pyrazol-1-yl]-3-fluoro-piperidine-1-carboxylate for tert-butyl N-[4-[3-cyano-4-[(3-fluoro-2-pyridyl)sulfanyl]pyrazolo[1,5-a]pyridin-6-yl]-2-methyl-pyrazol-3-yl]carbamate to give 6-(5-amino-1-methyl-pyrazol-4-yl)-4-[(3-fluoro-2-pyridyl)sulfanyl]pyrazolo[1,5-a]pyridine-3-carbonitrile (25 mg, 35% yield) was obtained as a white solid. LCMS (MM-ES+APCI, Pos): m/z 366.2 (M+H).

### Example 68

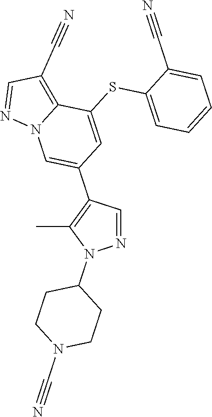 [SMILES: Cc1c(-c2cc(Sc3ccccc3C#N)c3c(C#N)cnn3c2)cnn1C1CCN(C#N)CC1]

### 4-(2-cyanophenyl)sulfanyl-6-[1-(1-cyano-4-piperidyl)-5-methyl-pyrazol-4-yl]pyrazolo[1,5-a]pyridine-3-carbonitrile (556)

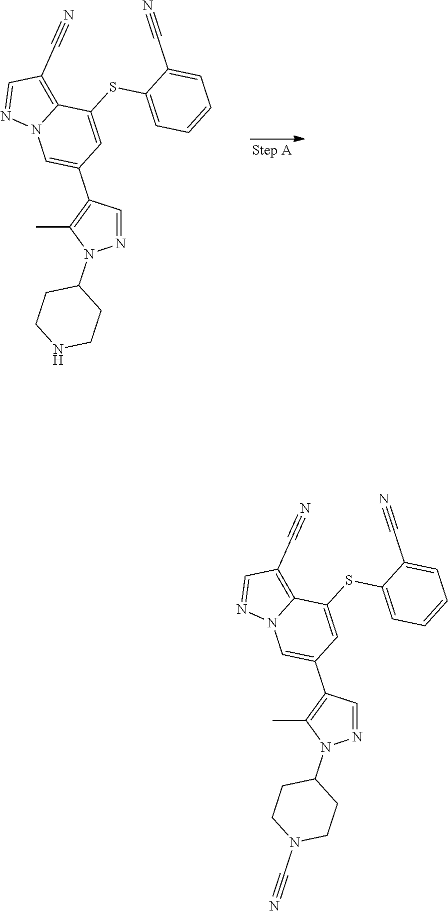 [SMILES: Cc1c(-c2cc(Sc3ccccc3C#N)c3c(C#N)cnn3c2)cnn1C1CCNCC1 ; Cc1c(-c2cc(Sc3ccccc3C#N)c3c(C#N)cnn3c2)cnn1C1CCN(C#N)CC1]

Step A. 4-(2-cyanophenyl)sulfanyl-6-[1-(1-cyano-4-piperidyl)-5-methyl-pyrazol-4-yl]pyrazolo[1,5-a]pyridine-3-carbonitrile. To a 40 mL vial equipped with a magnetic stir bar was added 4-(2-cyanophenyl)sulfanyl-6-[5-methyl-1-(4-piperidyl)pyrazol-4-yl]pyrazolo[1,5-a]pyridine-3-carbonitrile (176 mg, 0.40 mmol) and K₂CO₃ (221 mg, 1.60 mmol) followed by the addition of ACN (3 mL). The solution was cooled to 0° C. Carbononitridic bromide (0.12 mL, 1.70 mmol) in ACN (1 mL) was added dropwise. The mixture was allowed to warm to 25° C. and stir for 4 h. Saturated aqueous sodium bicarbonate (4 mL) was added to the reaction and the mixture was transferred to a separatory funnel. The mixture was extracted with ethyl acetate (10 mL×3). The combined organic layers were washed with brine (15 mL), dried over anhydrous sodium sulfate, filtered, concentrated and purified by preparative HPLC (35%-65% acetonitrile/0.04% aqueous ammonia hydroxide+10 Mm NH₄HCO₃) After lyophilization, 4-(2-cyanophenyl)sulfanyl-6-[1-(1-cyano-4-piperidyl)-5-methyl-pyrazol-4-yl]pyrazolo[1,5-a]pyridine-3-carbonitrile (106 mg, 57% yield) was obtained as a white solid. LCMS (MM-ES+APCI, Pos): m/z 465.2 (M+H).

### Example 69

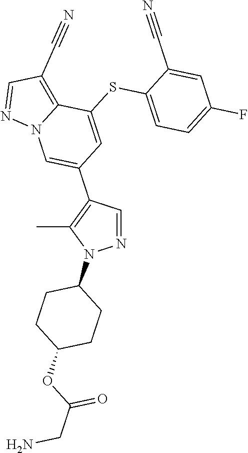 [SMILES: Cc1c(-c2cc(Sc3ccc(F)cc3C#N)c3c(C#N)cnn3c2)cnn1[C@H]1CC[C@H](OC(=O)CN)CC1]

### [4-[4-[3-cyano-4-(2-cyano-4-fluoro-phenyl)sulfanyl-pyrazolo[1,5-a]pyridin-6-yl]-5-methyl-pyrazol-1-yl]cyclohexyl]2-aminoacetate (636)

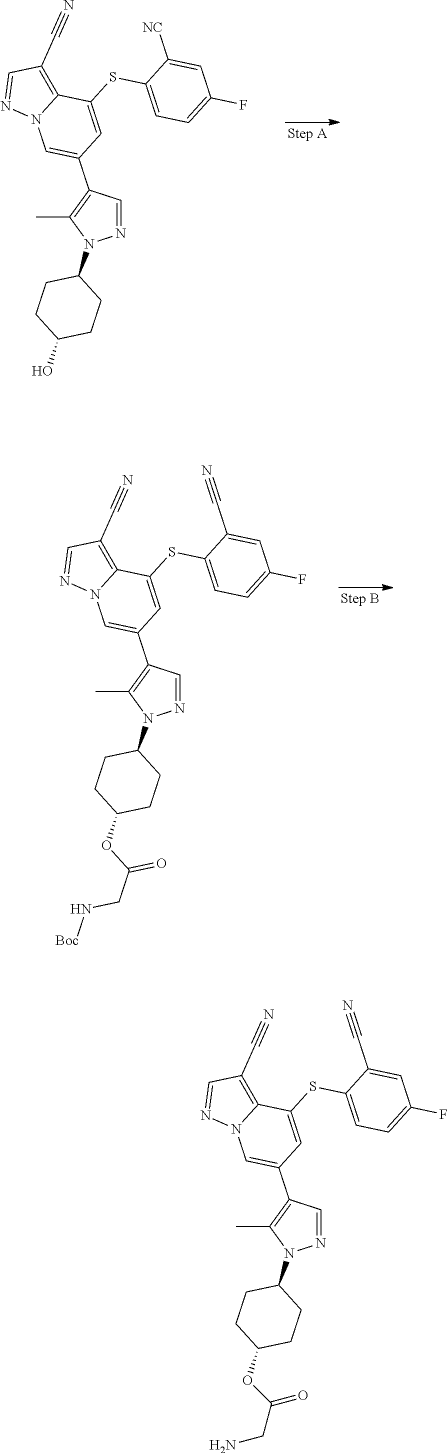 [SMILES: Cc1c(-c2cc(Sc3ccc(F)cc3C#N)c3c(C#N)cnn3c2)cnn1[C@H]1CC[C@H](O)CC1 ; Cc1c(-c2cc(Sc3ccc(F)cc3C#N)c3c(C#N)cnn3c2)cnn1[C@H]1CC[C@H](OC(=O)CNC(=O)OC(C)(C)C)CC1 ; Cc1c(-c2cc(Sc3ccc(F)cc3C#N)c3c(C#N)cnn3c2)cnn1[C@H]1CC[C@H](OC(=O)CN)CC1]

Step A. [4-[4-[3-cyano-4-(2-cyano-4-fluoro-phenyl)sulfanyl-pyrazolo[1,5-a]pyridin-6-yl]-5-methyl-pyrazol-1-yl]cyclohexyl]2-(tert-butoxycarbonylamino)acetate. To a 10 mL three-necked round-bottom flask equipped with a magnetic stir bar was added 2-(tertbutoxycarbonylamino)acetic acid (68.83 mg, 0.39 mmol), DIEA (0.2 mL, 1.18 mmol), DMAP (48.00 mg, 0.39 mmol, 2 eq) and EDCI (75.33 mg, 0.39 mmol) followed by the addition of DMF (3 mL). The solution was stirred at 25° C. for 0.1 h. 4-(2-Cyano-4-fluoro-phenyl)sulfanyl-6-[1-(4-hydroxycyclohexyl)-5-methyl-pyrazol-4-yl]pyrazolo[1,5-a]pyridine-3-carbonitrile (100 mg, 0.19 mmol) was added. The mixture was stirred at 25° C. for 12 h. Water (5 mL) was added to the reaction and the mixture was transferred to a separatory funnel. The mixture was extracted with ethyl acetate (5 mL×3). The combined organic layers were washed with brine (5 mL), dried over anhydrous sodium sulfate, filtered, concentrated and purified by silica gel column chromatography (0-50% petroleum ether/ethyl acetate) to give [4-[4-[3-cyano-4-(2-cyano-4-fluoro-phenyl)sulfanyl-pyrazolo[1,5-a]pyridin-6-yl]-5-methyl-pyrazol-1-yl]cyclohexyl]2-(tert-butoxycarbonylamino)acetate (110 mg, 89% yield) as a white solid. LCMS (MM-ES+APCI, Pos): m/z 630.1 (M+H).

Step B. [4-[4-[3-cyano-4-(2-cyano-4-fluoro-phenyl)sulfanyl-pyrazolo[1,5-a]pyridin-6-yl]-5-methyl-pyrazol-1-yl]cyclohexyl]2-aminoacetate. Synthesized according to Example 51, Step F, substituting tert-butyl (3S,4S)-4-[4-[3-cyano-4-[(3-fluoro-2-pyridyl)sulfanyl]pyrazolo[1,5-a]pyridin-6-yl]-5-methyl-pyrazol-1-yl]-3-fluoro-piperidine-1-carboxylate for [4-[4-[3-cyano-4-(2-cyano-4-fluorophenyl)sulfanyl-pyrazolo[1,5-a]pyridin-6-yl]-5-methyl-pyrazol-1-yl]cyclohexyl] 2-(tert-butoxycarbonylamino)acetate to give [4-[4-[3-cyano-4-(2-cyano-4-fluoro-phenyl)sulfanyl-pyrazolo[1,5-a]pyridin-6-yl]-5-methyl-pyrazol-1-yl]cyclohexyl]2-aminoacetate (60 mg, 68% yield) as a white solid. LCMS (MM-ES+APCI, Pos): m/z 530.3 (M+H).

### Example 70

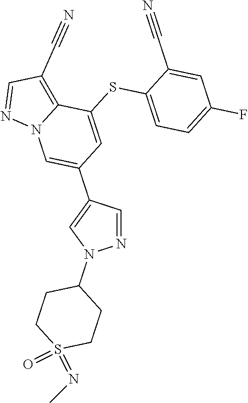 [SMILES: CN=S1(=O)CCC(n2cc(-c3cc(Sc4ccc(F)cc4C#N)c4c(C#N)cnn4c3)cn2)CC1]

### 4-(2-cyano-4-fluoro-phenyl)sulfanyl-6-[1-(1-methylimino-1-oxo-thian-4-yl)pyrazol-4-yl]pyrazolo[1,5-a]pyridine-3-carbonitrile (658)

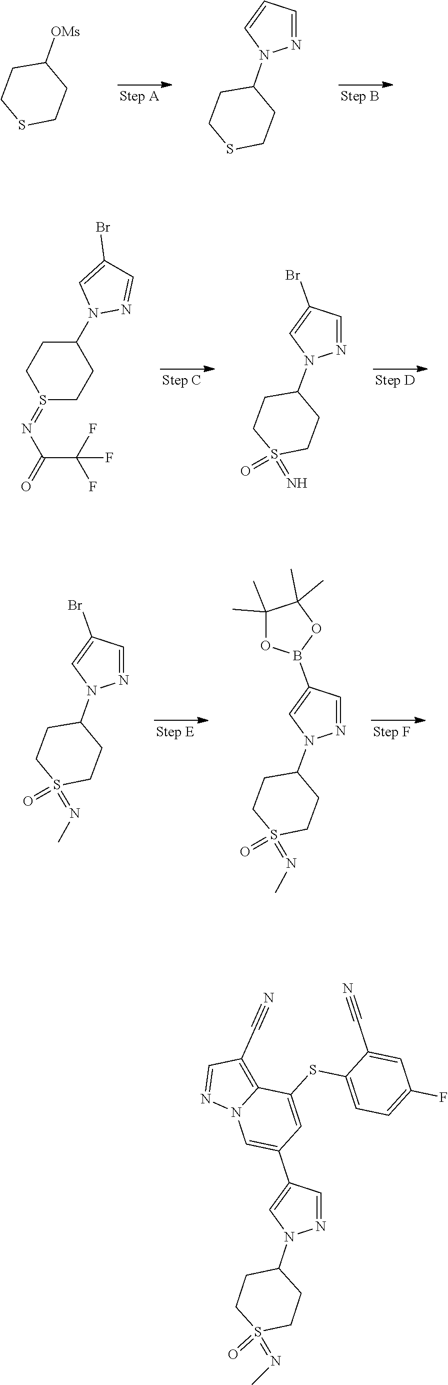

Step A. 1-tetrahydrothiopyran-4-ylpyrazole. Synthesized according to Example 51, Step B, substituting tert-butyl (3S,4R)-3-fluoro-4-methylsulfonyloxy-piperidine-1-carboxylate for tetrahydrothiopyran-4-yl methanesulfonate and 4-iodo-1H-pyrazole for 1H-pyrazole to give 1-tetrahydrothiopyran-4-ylpyrazole (1.5 g, 60% yield) as a brown solid. LCMS (MM-ES+APCI, Pos): m/z 169.0 (M+H).

Step B. N-[4-(4-bromopyrazol-1-yl)thian-1-ylidene]-2,2,2-trifluoro-acetamide. To a 100 mL three-necked round-bottom flask equipped with a magnetic stir bar was added 1-tetrahydrothiopyran-4-ylpyrazole (1.10 g, 6.28 mmol) and 2,2,2-trifluoroacetamide (710 mg, 6.28 mmol) followed by the addition of MTBE (7 mL). The solution was cooled to 0° C. t-BuOK (1.10 g, 9.41 mmol) was added in portions, the reaction was stirred at 0° C. for 1 h. A solution of 1,3-dibromo-5,5-dimethyl-imidazolidine-2,4-dione (4.50 g, 15.7 mmol) in THF (6 mL) was added dropwise. The mixture was stirred at 0° C. for 1 h. Saturated aqueous sodium bisulfite (15 mL) was added to the reaction and the mixture was transferred to a separatory funnel. The mixture was extracted with ethyl acetate (10 mL×3). The combined organic layers were washed with brine (15 mL), dried over anhydrous sodium sulfate, filtered, concentrated and purified by silica gel column chromatography (25-30% petroleum ether/ethyl acetate) to give N-[4-(4-bromopyrazol-1-yl)thian-1-ylidene]-2,2,2-trifluoro-acetamide (500 mg, 22% yield) as a light yellow solid.

Step C. 4-(4-bromopyrazol-1-yl)-1-imino-thiane 1-oxide. To a 40 mL vial equipped with magnetic stir bar was added N-[4-(4-bromopyrazol-1-yl)thian-1-ylidene]-2,2,2-trifluoro-acetamide (500 mg, 1.40 mmol) followed by the addition of MeOH (2.5 mL), H₂O (0.4 mL, 21.6 mmol), K₂CO₃ (386 mg, 2.79 mmol) and ACN (2.5 mL). 30% H₂O₂ (0.67 mL, 6.98 mmol) was added dropwise into the mixture at 20° C. The mixture was stirred at 20° C. for 2 h. Saturated aqueous sodium bisulfite (10 mL) was added to the reaction and the mixture stirred for 0.5 h. The mixture was diluted with ethyl acetate (10 mL), the resulting mixture was transferred to a separatory funnel, and the aqueous layer mixture was extracted with ethyl acetate (10 mL×3). The combined organic layers were washed with brine (10 mL), dried over anhydrous sodium sulfate, filtered, and concentrated under reduced pressure affording the crude product 4-(4-bromopyrazol-1-yl)-1-imino-thiane 1-oxide (350 mg, 88% yield) as a white solid, and used into the next step without further purification.

Step D. 4-(4-bromopyrazol-1-yl)-1-methylimino-thiane 1-oxide To a 40 mL vial equipped with a magnetic stir bar was added 4-(4-bromopyrazol-1-yl)-1-imino-thiane 1-oxide (350 mg, 1.23 mmol, TFA (0.9 mL, 12.27 mmol), (HCHO)n (395 mg, 12.3 mmol) and triethylsilane (2 mL, 12.3 mmol) followed by the addition of ACN (8 mL). The mixture was stirred at 25° C. for 16 h. The pH of mixture was adjusted to 8 by using saturated aqueous sodium bicarbonate. The mixture was concentrated under reduced pressure, water was added (20 mL) and transferred to a separatory funnel, the aqueous layer mixture was extracted with DCM (15 mL×3). The combined organic layers were washed with brine (20 mL), dried over anhydrous sodium sulfate, filtered, and concentrated under reduced pressure affording the crude product 4-(4-bromopyrazol-1-yl)-1-methylimino-thiane 1-oxide (440 mg, crude) as a white solid and used into the next step without further purification.

Step E. 1-methylimino-4-[4-(4,4,5,5-tetramethyl-1,3,2-dioxaborolan-2-yl)pyrazol-1-yl]thiane 1-oxide. To a 20 mL Schlenk tube equipped with a magnetic stir bar was added 4-(4-bromopyrazol-1-yl)-1-methylimino-thiane-1-oxide (200 mg, 0.68 mmol) and 4,4,5,5-tetramethyl-2-(4,4,5,5-tetramethyl-1,3,2-dioxaborolan-2-yl)-1,3,2-dioxaborolane (261 mg, 1.03 mmol) followed by the addition of dioxane (3 mL). Pd(dppf)Cl₂ (50.0 mg, 0.07 mmol) and KOAc (336 mg, 3.42 mmol) was added into the mixture at 25° C. The tube was then evacuated and backfilled with nitrogen three times. The mixture was heated to 90° C. and stirred for 1.5 h. The reaction solution was used into the next step without work-up and purification. 1-methylimino-4-[4-(4,4,5,5-tetramethyl-1,3,2-dioxaborolan-2-yl)pyrazol-1-yl]thiane 1-oxide (232.2 mg, crude) in dioxane (3 mL) was obtained as a yellow solution and used in the next step without further purification.

Step F. 4-(2-cyano-4-fluoro-phenyl)sulfanyl-6-[1-(1-methylimino-1-oxo-thian-4-yl)pyrazol-4-yl]pyrazolo[1,5-a]pyridine-3-carbonitrile. Synthesized according to Example 51, Step E, substituting tert-butyl (3S,4S)-3-fluoro-4-[5-methyl-4-(4,4,5,5-tetramethyl-1,3,2-dioxaborolan-2-yl)pyrazol-1-yl]piperidine-1-carboxylate for 1-methylimino-4-[4-(4,4,5,5-tetramethyl-1,3,2-dioxaborolan-2-yl)pyrazol-1-yl]thiane 1-oxide to give 4-(2-cyano-4-fluoro-phenyl)sulfanyl-6-[1-(1-methylimino-1-oxo-thian-4-yl)pyrazol-4-yl]pyrazolo[1,5-a]pyridine-3-carbonitrile (42.9 mg, 7% yield) as an off-white solid. LCMS (MM-ES+APCI, Pos): m/z 506.2 (M+H).

### Example 71

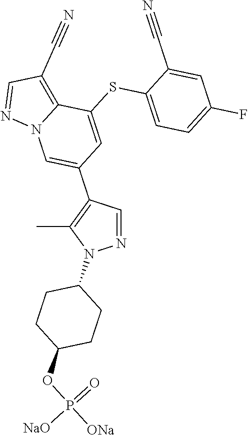

### [[4-[4-[3-cyano-4-(2-cyano-4-fluoro-phenyl)sulfanyl-pyrazolo[1,5-a]pyridin-6-yl]-5-methyl-pyrazol-1-yl]cyclohexoxy]-sodiooxy-phosphoryl]oxysodium (679)

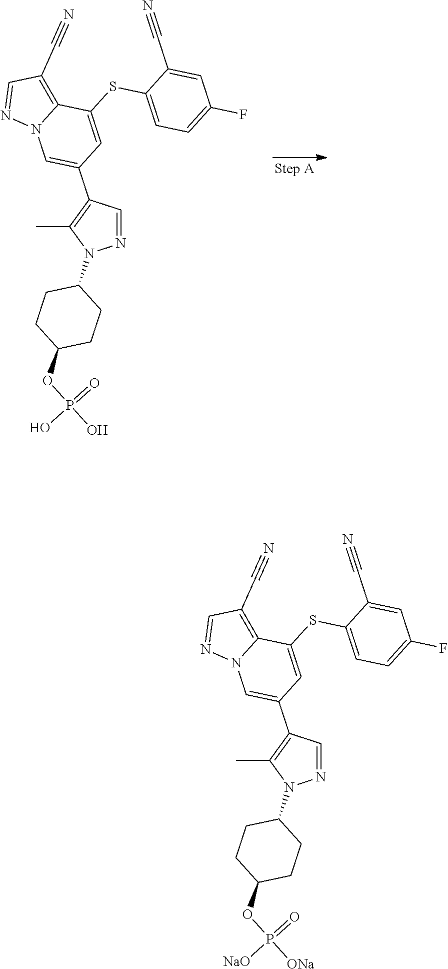

Step A. [[4-[4-[3-cyano-4-(2-cyano-4-fluoro-phenyl)sulfanyl-pyrazolo[1,5-a]pyridin-6-yl]-5-methyl-pyrazol-1-yl]cyclohexoxy]-sodiooxy-phosphoryl]oxysodium. To a 250 mL flask equipped with a magnetic stir bar was added [4-[4-[3-cyano-4-(2-cyano-4-fluoro-phenyl)sulfanyl-pyrazolo[1,5-a]pyridin-6-yl]-5-methyl-pyrazol-1-yl]cyclohexyl] dihydrogen phosphate (700 mg, 1.19 mmol) followed by the addition of MeCN (4 mL) and H₂O (4 mL). The solution was cooled to 0° C. NaOH (1 M, 2.39 mL, 2.38 mmol) was added dropwise. The mixture was allowed to warm to 25° C. and stir for 5 min. The crude product was added H₂O (50 mL) and acetonitrile (5 mL). After lyophilization, the crude product was purified by slurry in MeCN (20 mL) at 25° C. for 0.5 h. After filtration and drying under vacuum, the desired product was obtained as a white solid, [[4-[4-[3-cyano-4-(2-cyano-4-fluoro-phenyl)sulfanyl-pyrazolo[1,5-a]pyridin-6-yl]-5-methyl-pyrazol-1-yl]cyclohexoxy]-sodiooxy-phosphoryl]oxysodium (750 mg, 97% yield). LCMS (MM-ES+APCI, Pos): m/z 553.0 (M+H).

| | | | | MS | Enz |
|---|---|---|---|---|---|
| Compound | | | Reference | APCI | FGFR2 |
| number | Structure | Chemical Name | Procedure | (m/z) | (nM) |
| 50 | 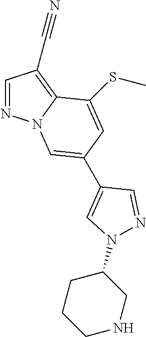 [SMILES: CSc1cc(-c2cnn([C@H]3CCCNC3)c2)cn2ncc(C#N)c12] | 4-methylsulfanyl-6-[1-[(3S)- 3-piperidyl]pyrazol-4-yl] pyrazolo[1,5-a]pyridine-3- carbonitrile | Example 1 | 339.0 | 2 |
| 51 | 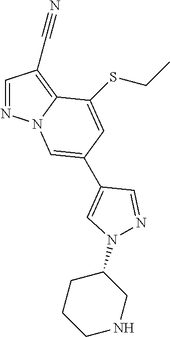 [SMILES: CCSc1cc(-c2cnn([C@H]3CCCNC3)c2)cn2ncc(C#N)c12] | 4-ethylsulfanyl-6-[1-[(3S)-3- piperidyl]pyrazol-4- yl]pyrazolo[1,5-a]pyridine-3- carbonitrile | Example 1 | 353.2 | 1 |
| 52 | 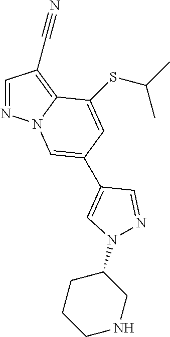 [SMILES: CC(C)Sc1cc(-c2cnn([C@H]3CCCNC3)c2)cn2ncc(C#N)c12] | 4-isopropylsulfanyl-6-[1- [(3S)-3-piperidyl]pyrazol-4- yl]pyrazolo[1,5-a]pyridine-3- carbonitrile | Example 1 | 367.2 | 2 |
| 53 | 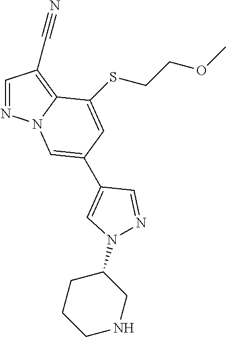 [SMILES: COCCSc1cc(-c2cnn([C@H]3CCCNC3)c2)cn2ncc(C#N)c12] | 4-(2- methoxyethylsulfanyl)-6-[1- [(3S)-3-piperidyl] pyrazol- 4-yl]pyrazolo[1,5-a]pyridine- 3-carbonitrile | Example 10 | 383.2 | 3 |
| 54 | 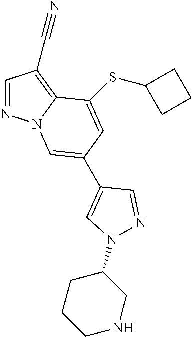 [SMILES: N#Cc1cnn2cc(-c3cnn([C@H]4CCCNC4)c3)cc(SC3CCC3)c12] | 4-cyclobutylsulfanyl-6-[1- [(3S)-3-piperidyl]pyrazol-4- yl]pyrazolo[1,5-a]pyridine-3- carbonitrile | Example 10 | 379.3 | 1 |
| 55 | 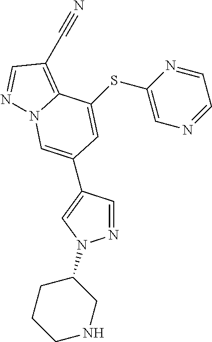 [SMILES: N#Cc1cnn2cc(-c3cnn([C@H]4CCCNC4)c3)cc(Sc3cnccn3)c12] | 6-[1-[(3S)-3- piperidyl]pyrazol-4-yl]-4- pyrazin-2-ylsulfanyl- pyrazolo[1,5-a]pyridine-3- carbonitrile | Example 1 | 403.3 | 13 |
| 56 | 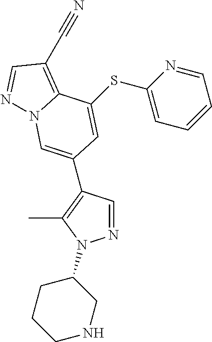 [SMILES: Cc1c(-c2cc(Sc3ccccn3)c3c(C#N)cnn3c2)cnn1[C@H]1CCCNC1] | 6-[5-methyl-1-[(3S)-3- piperidyl]pyrazol-4-yl]-4-(2- pyridylsulfanyl)pyrazolo[1,5- a]pyridine-3-carbonitrile | Example 1 | 416.3 | 6 |
| 57 | 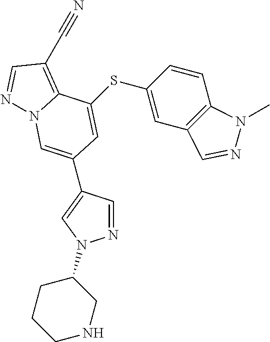 [SMILES: Cn1ncc2cc(Sc3cc(-c4cnn([C@H]5CCCNC5)c4)cn4ncc(C#N)c34)ccc21] | 4-(1-methylindazol-5- yl)sulfanyl-6-[1-[(3S)-3- piperidyl]pyrazol-4- yl]pyrazolo[1,5-a]pyridine-3- carbonitrile | Example 1 | 455.2 | 13 |
| 58 | 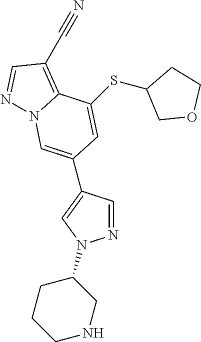 [SMILES: N#Cc1cnn2cc(-c3cnn([C@H]4CCCNC4)c3)cc(SC3CCOC3)c12] | 6-[1-[(3S)-3- piperidyl]pyrazol-4-yl]-4- tetrahydrofuran-3-ylsulfanyl- pyrazolo[1,5-a]pyridine-3- carbonitrile | Example 10 | 395.1 | 4 |
| 59 | 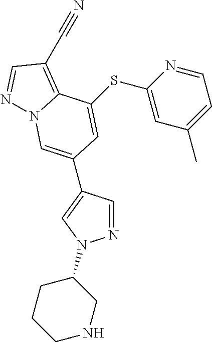 [SMILES: Cc1ccnc(Sc2cc(-c3cnn([C@H]4CCCNC4)c3)cn3ncc(C#N)c23)c1] | 4-[(4-methyl-2-pyridyl) sulfanyl]-6-[1-[(3S)-3- piperidyl]pyrazol-4- yl]pyrazolo[1,5-a]pyridine-3- carbonitrile | Example 1 | 416.0 | 11 |
| 60 | 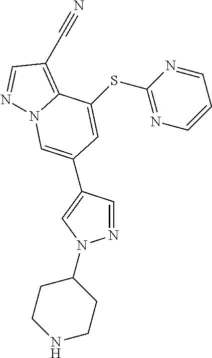 [SMILES: N#Cc1cnn2cc(-c3cnn(C4CCNCC4)c3)cc(Sc3ncccn3)c12] | 6-(1-(piperidin-4-yl)-1H- pyrazol-4-yl)-4-(pyrimidin- 2-ylthio)pyrazolo[1,5- a]pyridine-3-carbonitrile, HCl | Example 1 | 403.2 | 7 |
| 61 | 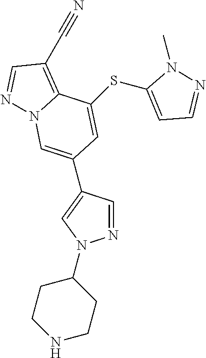 [SMILES: Cn1nccc1Sc1cc(-c2cnn(C3CCNCC3)c2)cn2ncc(C#N)c12] | 4-((1-methyl-1H-pyrazol-5- yl)thio)-6-(1-(piperidin-4- yl)-1H-pyrazol-4- yl)pyrazolo[1,5-a]pyridine-3- carbonitrile, HCl | Example 1 | 405.2 | 9 |
| 62 | 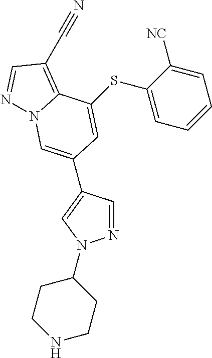 [SMILES: N#Cc1ccccc1Sc1cc(-c2cnn(C3CCNCC3)c2)cn2ncc(C#N)c12] | 4-((2-cyanophenyl)thio)-6- (1-(piperidin-4-yl)-1H- pyrazol-4-yl)pyrazolo[1,5- a]pyridine-3-carbonitrile | Example 1 | 426.2 | 0.3 |
| 63 | 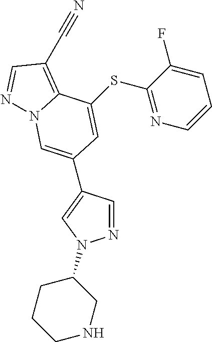 [SMILES: N#Cc1cnn2cc(-c3cnn([C@H]4CCCNC4)c3)cc(Sc3ncccc3F)c12] | 4-[(3-fluoro-2- pyridyl)sulfanyl]-6-[1-[(3S)- 3-piperidyl]pyrazol-4- yl]pyrazolo[1,5-a]pyridine-3- carbonitrile | Example 1 | 420.3 | 1 |
| 64 | 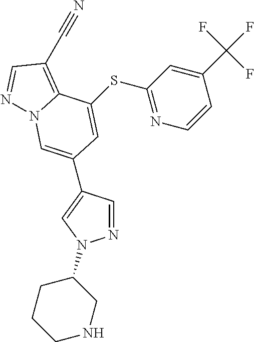 [SMILES: N#Cc1cnn2cc(-c3cnn([C@H]4CCCNC4)c3)cc(Sc3cc(C(F)(F)F)ccn3)c12] | 6-[1-[(3S)-3- piperidyl]pyrazol-4-y1]-4- [[4-(trifluoromethyl)-2- pyridyl]sulfanyl]pyrazolo[1, 5-a]pyridine-3-carbonitrile | Example 1 | 470.3 | 61 |
| 65 | 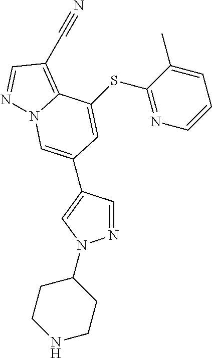 [SMILES: Cc1cccnc1Sc1cc(-c2cnn(C3CCNCC3)c2)cn2ncc(C#N)c12] | 4-((3-methylpyridin-2- yl)thio)-6-(1-(piperidin-4- yl)-1H-pyrazol-4- yl)pyrazolo[1,5-a]pyridine-3- carbonitrile, HCl | Example 1 | 416.3 | 4 |
| 66 | 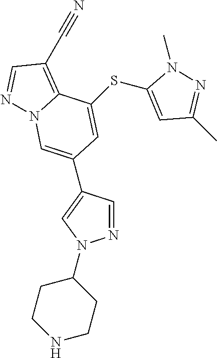 [SMILES: Cc1cc(Sc2cc(-c3cnn(C4CCNCC4)c3)cn3ncc(C#N)c23)n(C)n1] | 4-((1,3-dimethyl-1H- pyrazol-5-yl)thio)-6-(1- (piperidin-4-yl)-1H-pyrazol- 4-yl)pyrazolo[1,5-a]pyridine- 3-carbonitrile, HCl | Example 1 | 419.3 | 12 |
| Example 67 | 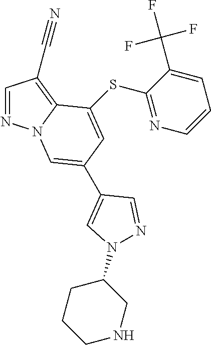 [SMILES: N#Cc1cnn2cc(-c3cnn([C@H]4CCCNC4)c3)cc(Sc3ncccc3C(F)(F)F)c12] | 6-[1-[(3S)-3- piperidyl]pyrazol-4-yl]-4- [3-(trifluoromethyl)-2- pyridyl]sulfanyl]pyrazolo[1, 5-a]pyridine-3-carbonitrile | Example 1 | 470.1 | 4 |
| Example 68 | 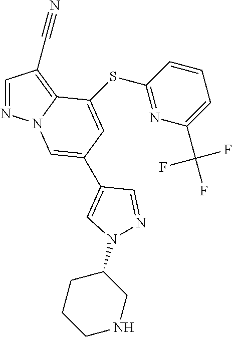 [SMILES: N#Cc1cnn2cc(-c3cnn([C@H]4CCCNC4)c3)cc(Sc3cccc(C(F)(F)F)n3)c12] | 6-[1-[(3S)-3- piperidyl]pyrazol-4-yl]-4- [[6-(trifluoromethyl)-2- pyridyl]sulfanyl] pyrazolo[1,5-a]pyridine-3- carbonitrile | Example 1 | 470.2 | 68 |
| 69 | 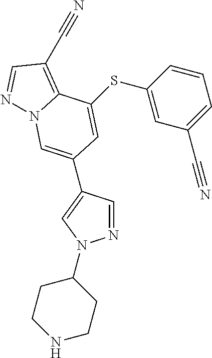 [SMILES: N#Cc1cccc(Sc2cc(-c3cnn(C4CCNCC4)c3)cn3ncc(C#N)c23)c1] | 4-((3-cyanophenyl)thio)-6- (1-(piperidin-4-yl)-1H- pyrazol-4-yl)pyrazolo[1,5- a]pyridine-3-carbonitrile | Example 1 | 426.2 | 8 |
| 70 | 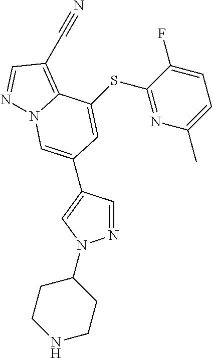 [SMILES: Cc1ccc(F)c(Sc2cc(-c3cnn(C4CCNCC4)c3)cn3ncc(C#N)c23)n1] | 4-((3-fluoro-6- methylpyridin-2-yl)thio)-6- (1-(piperidin-4-yl)-1H- pyrazol-4-yl)pyrazolo[1,5- a]pyridine-3-carbonitrile, HCl | Example 10 | 434.3 | 3 |
| 71 | 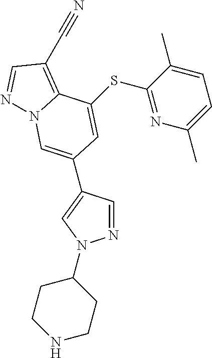 [SMILES: Cc1ccc(C)c(Sc2cc(-c3cnn(C4CCNCC4)c3)cn3ncc(C#N)c23)n1] | 4-((3,6-dimethylpyridin-2- yl)thio)-6-(1-(piperidin-4- yl)-1H-pyrazol-4- yl)pyrazolo[1,5-a]pyridine-3- carbonitrile, HCl | Example 10 | 416.3 | 8 |
| 72 | 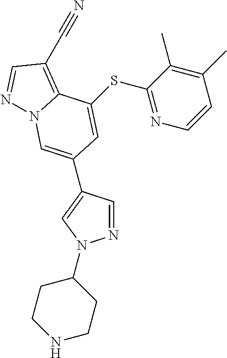 [SMILES: Cc1ccnc(Sc2cc(-c3cnn(C4CCNCC4)c3)cn3ncc(C#N)c23)c1C] | 4-((3,4-dimethylpyridin-2- yl)thio)-6-(1-(piperidin-4- yl)-1H-pyrazol-4- yl)pyrazolo[1,5-a]pyridine-3- carbonitrile | Example 10 | 430.3 | 3 |
| 73 | 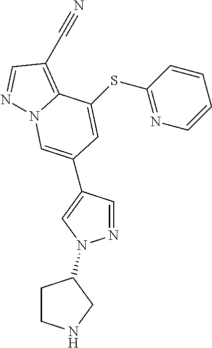 | (S)-4-(pyridin-2-ylthio)-6-(1- (pyrrolidin-3-yl)-1H- pyrazol-4-yl)pyrazolo[1,5- a]pyridine-3-carbonitrile, HCl | Example 1 | 388.2 | 4 |
| 74 | 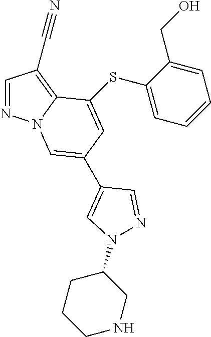 [SMILES: N#Cc1cnn2cc(-c3cnn([C@H]4CCCNC4)c3)cc(Sc3ccccc3CO)c12] | 4-[2- (hydroxymethyl)phenyl]sulfa- nyl-6-[1-[(3S)-3- piperidyl]pyrazol-4- yl]pyrazolo[1,5-a]pyridine-3- carbonitrile | Example 1 | 431.3 | 6 |
| 75 | 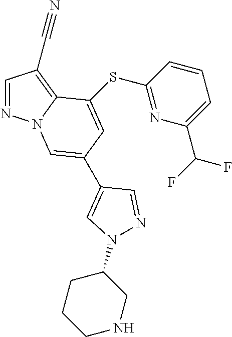 [SMILES: N#Cc1cnn2cc(-c3cnn([C@H]4CCCNC4)c3)cc(Sc3cccc(C(F)F)n3)c12] | 4-[[6-(difluoromethyl)-2- pyridyl]sulfanyl]-6-[1-[(3S)- 3-piperidyl]pyrazol-4- yl]pyrazolo[1,5-a]pyridine-3- carbonitrile | Example 1 | 452.3 | 22 |
| 76 | 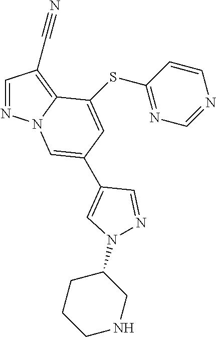 [SMILES: N#Cc1cnn2cc(-c3cnn([C@H]4CCCNC4)c3)cc(Sc3ccncn3)c12] | 6-[1-[(3S)-3- piperidyl]pyrazol-4-yl]-4- pyrimidin-4-ylsulfanyl- pyrazolo[1,5-a]pyridine-3- carbonitrile | Example 1 | 403.1 | 237 |
| 77 | 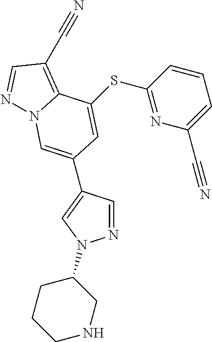 [SMILES: N#Cc1cccc(Sc2cc(-c3cnn([C@H]4CCCNC4)c3)cn3ncc(C#N)c23)n1] | 4-[(6-cyano-2-pyridyl) sulfanyl]-6-[1-[(3S)-3- piperidyl]pyrazol-4- yl]pyrazolo[1,5-a]pyridine-3- carbonitrile | Example 10 | 462.9 | 99 |
| 78 | 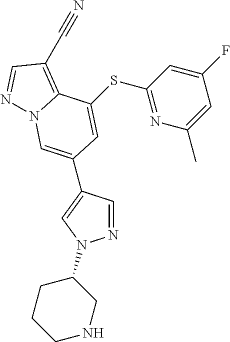 [SMILES: Cc1cc(F)cc(Sc2cc(-c3cnn([C@H]4CCCNC4)c3)cn3ncc(C#N)c23)n1] | 4-[(4-fluoro-6-methyl-2- pyridyl)sulfanyl]-6-[1-[(3S)- 3-piperidyl]pyrazol-4- yl]pyrazolo[1,5-a]pyridine-3- carbonitrile | Example 1 | 434.4 | 27 |
| 79 | 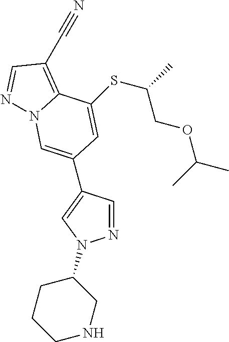 [SMILES: CC(C)OC[C@@H](C)Sc1cc(-c2cnn([C@H]3CCCNC3)c2)cn2ncc(C#N)c12] | 4-[(1R)-2-isopropoxy-1- methyl-ethyl]sulfanyl-6-[1- [(3S)-3-piperidyl]pyrazol-4- yl]pyrazolo[1,5-a]pyridine-3- carbonitrile | Example 10 | 425.5 | 2 |
| 80 | 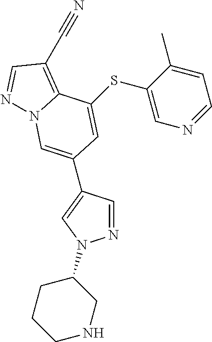 [SMILES: Cc1ccncc1Sc1cc(-c2cnn([C@H]3CCCNC3)c2)cn2ncc(C#N)c12] | 4-[(4-methyl-3- pyridyl)sulfanyl]-6-[1-[(3S)- 3-piperidyl]pyrazol-4- yl]pyrazolo[1,5-a]pyridine-3- carbonitrile | Example 1 | 415.9 | 8 |
| 81 | 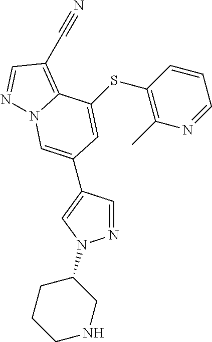 [SMILES: Cc1ncccc1Sc1cc(-c2cnn([C@H]3CCCNC3)c2)cn2ncc(C#N)c12] | 4-[(2-methyl-3- pyridyl)sulfanyl]-6-[1-[(3S)- 3-piperidyl]pyrazol-4- yl]pyrazolo[1,5-a]pyridine-3- carbonitrile | Example 1 | 416.2 | 31 |
| 82 | 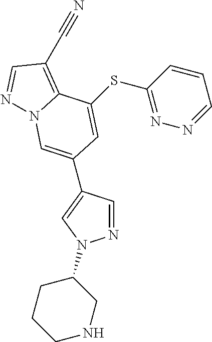 [SMILES: N#Cc1cnn2cc(-c3cnn([C@H]4CCCNC4)c3)cc(Sc3cccnn3)c12] | 6-[1-[(3S)-3- piperidyl]pyrazol-4-yl]-4- pyridazin-3-ylsulfanyl- pyrazolo[1,5-a]pyridine-3- carbonitrile | Example 1 | 403.5 | 32 |
| 83 | 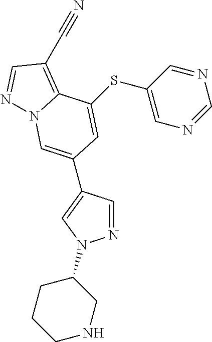 [SMILES: N#Cc1cnn2cc(-c3cnn([C@H]4CCCNC4)c3)cc(Sc3cncnc3)c12] | 6-[1-[(3S)-3-piperidyl] pyrazol-4-yl]-4-pyrimidin-5- ylsulfanyl-pyrazolo[1,5-a] pyridine-3-carbonitrile | Example 1 | 403.1 | 19 |
| 84 | 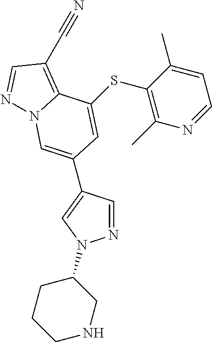 [SMILES: Cc1ccnc(C)c1Sc1cc(-c2cnn([C@H]3CCCNC3)c2)cn2ncc(C#N)c12] | 4-[(2,4-dimethyl-3- pyridyl)sulfanyl]-6-[1-[(3S)- 3-piperidyl]pyrazol-4- yl]pyrazolo[1,5-a]pyridine- 3-carbonitrile | Example 1 | 430.2 | 11 |
| 85 | 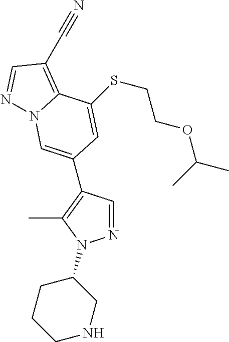 [SMILES: Cc1c(-c2cc(SCCOC(C)C)c3c(C#N)cnn3c2)cnn1[C@H]1CCCNC1] | 4-(2- isopropoxyethylsulfanyl)-6- [5-methyl-1-[(3S)-3- piperidyl]pyrazol-4- yl]pyrazolo[1,5-a]pyridine-3- carbonitrile | Example 10 | 425.5 | 7 |
| 86 | 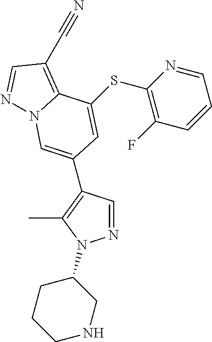 [SMILES: Cc1c(-c2cc(Sc3ncccc3F)c3c(C#N)cnn3c2)cnn1[C@H]1CCCNC1] | 4-[(3-fluoro-2- pyridyl)sulfanyl]-6-[5- methyl-1-[(3S)-3- piperidyl]pyrazol-4- yl]pyrazolo[1,5-a]pyridine-3- carbonitrile | Example 1 | 434.1 | 2 |
| 87 | 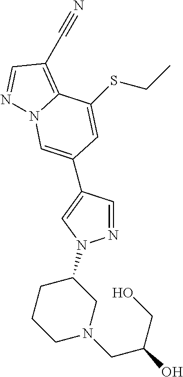 [SMILES: CCSc1cc(-c2cnn([C@H]3CCCN(C[C@H](O)CO)C3)c2)cn2ncc(C#N)c12] | 6-[1-[(3S)-1-[(2S)-2,3- dihydroxypropyl]-3- piperidyl]pyrazol-4-yl]-4- ethylsulfanyl-pyrazolo[1,5- a]pyridine-3-carbonitrile | Example 35 | 427.3 | 1 |
| 88 | 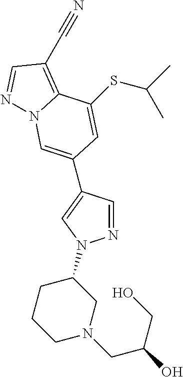 [SMILES: CC(C)Sc1cc(-c2cnn([C@H]3CCCN(C[C@H](O)CO)C3)c2)cn2ncc(C#N)c12] | 6-[1-[(3S)-1-[(2S)-2,3- dihydroxypropyl]-3- piperidyl]pyrazol-4-yl]-4- isopropylsulfanyl- pyrazolo[1,5-a]pyridine-3- carbonitrile | Example 35 | 441.2 | 1 |
| 89 | 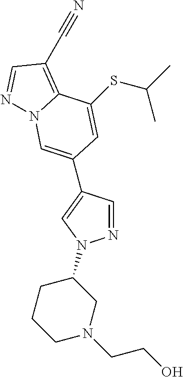 [SMILES: CC(C)Sc1cc(-c2cnn([C@H]3CCCN(CCO)C3)c2)cn2ncc(C#N)c12] | 6-[1-[(3S)-1-(2- hydroxyethyl)-3- piperidyl]pyrazol-4-yl]-4- isopropylsulfanyl- pyrazolo[1,5-a]pyridine-3- carbonitrile | Example 35 | 411.4 | 1 |
| 90 | 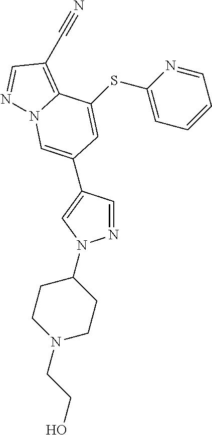 [SMILES: N#Cc1cnn2cc(-c3cnn(C4CCN(CCO)CC4)c3)cc(Sc3ccccn3)c12] | 6-[1-[1-(2-hydroxyethyl)-4- piperidyl]pyrazol-4-yl]-4-(2- pyridylsulfanyl)pyrazolo[1,5- a]pyridine-3-carbonitrile | Example 35 | 446.2 | 2 |
| 91 | 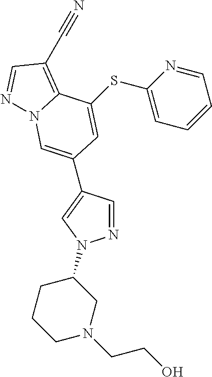 [SMILES: N#Cc1cnn2cc(-c3cnn([C@H]4CCCN(CCO)C4)c3)cc(Sc3ccccn3)c12] | 6-[1-[(3S)-1-(2- hydroxyethyl)-3- piperidyl]pyrazol-4-yl]-4-(2- pyridylsulfanyl)pyrazolo[1,5- a]pyridine-3-carbonitrile | Example 35 | 446.3 | 2 |
| 92 | 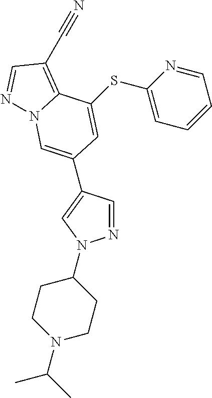 [SMILES: CC(C)N1CCC(n2cc(-c3cc(Sc4ccccn4)c4c(C#N)cnn4c3)cn2)CC1] | 6-[1-(1-isopropyl-4- piperidyl)pyrazol-4-yl]-4-(2- pyridylsulfanyl)pyrazolo[1,5- a]pyridine-3-carbonitrile | Example 32 | 444.1 | 3 |
| 93 | 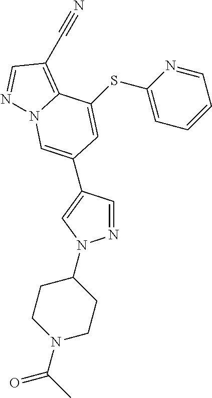 [SMILES: CC(=O)N1CCC(n2cc(-c3cc(Sc4ccccn4)c4c(C#N)cnn4c3)cn2)CC1] | 6-[1-(1-acetyl-4- piperidyl)pyrazol-4-yl]-4-(2- pyridylsulfanyl)pyrazolo [1,5-a]pyridine-3- carbonitrile | Example 36 | 444.1 | 3 |
| 94 | 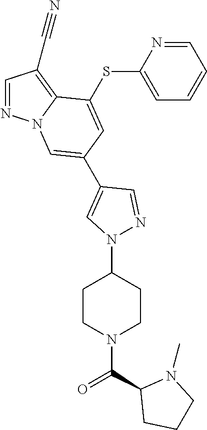 [SMILES: CN1CCC[C@H]1C(=O)N1CCC(n2cc(-c3cc(Sc4ccccn4)c4c(C#N)cnn4c3)cn2)CC1] | 6-[1-[1-[(2S)-1- methylpyrrolidine-2- carbonyl]-4- piperidyl]pyrazol-4-yl]-4-(2- pyridylsulfanyl) pyrazolo[1,5-a]pyridine-3- carbonitrile | Example 36 | 513.2 | 2 |
| 95 | 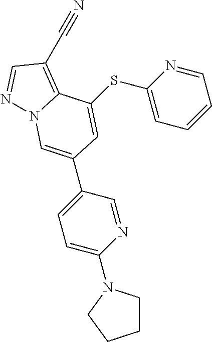 [SMILES: N#Cc1cnn2cc(-c3ccc(N4CCCC4)nc3)cc(Sc3ccccn3)c12] | 4-(2-pyridylsulfanyl)-6-(6- pyrrolidin-1-yl-3- pyridyl)pyrazolo[1,5- a]pyridine-3-carbonitrile | Example 24 | 399.1 | 13 |
| 96 | 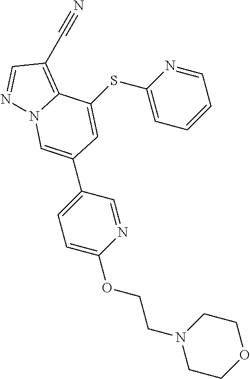 [SMILES: N#Cc1cnn2cc(-c3ccc(OCCN4CCOCC4)nc3)cc(Sc3ccccn3)c12] | 6-[6-(2-morpholinoethoxy)- 3-pyridyl]-4-(2- pyridylsulfanyl) pyrazolo[1,5-a]pyridine-3- carbonitrile | Example 24 | 459.3 | 82 |
| 97 | 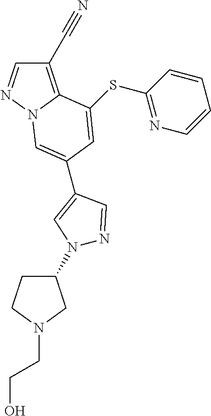 [SMILES: N#Cc1cnn2cc(-c3cnn([C@H]4CCN(CCO)C4)c3)cc(Sc3ccccn3)c12] | (S)-6-(1-(1-(2- hydroxyethyl)pyrrolidin-3- yl)-1H-pyrazol-4-yl)-4- (pyridin-2- ylthio)pyrazolo[1,5- a]pyridine-3-carbonitrile | Example 35 | 432.2 | 4 |
| 98 | 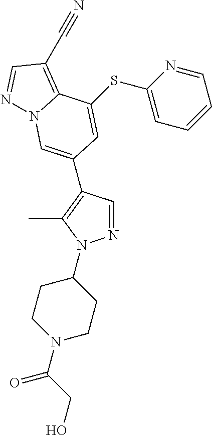 [SMILES: Cc1c(-c2cc(Sc3ccccn3)c3c(C#N)cnn3c2)cnn1C1CCN(C(=O)CO)CC1] | 6-(1-(1-(2- hydroxyacetyl)piperidin-4- yl)-5-methyl-1H-pyrazol-4- yl)-4-(pyridin-2- ylthio)pyrazolo[1,5- a]pyridine-3-carbonitrile | Example 36 | 474.2 | 7 |
| 99 | 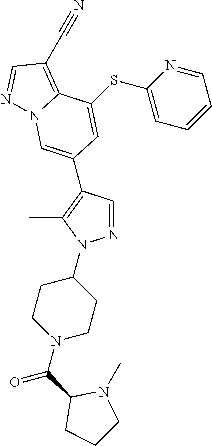 [SMILES: Cc1c(-c2cc(Sc3ccccn3)c3c(C#N)cnn3c2)cnn1C1CCN(C(=O)[C@@H]2CCCN2C)CC1] | 6-(5-methyl-1-(1-(methyl-L- prolyl)piperidin-4-yl)-1H- pyrazol-4-yl)-4-(pyridin-2- ylthio)pyrazolo[1,5- a]pyridine-3-carbonitrile | Example 1 | 527.3 | 5 |
| 100 | 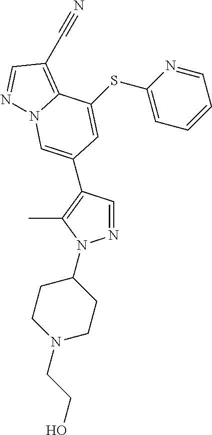 [SMILES: Cc1c(-c2cc(Sc3ccccn3)c3c(C#N)cnn3c2)cnn1C1CCN(CCO)CC1] | 6-(1-(1-(2- hydroxyethyl)piperidin-4- yl)-5-methyl-1H-pyrazol-4- yl)-4-(pyridin-2- ylthio)pyrazolo[1,5- a]pyridine-3-carbonitrile | Example 35 | 460.3 | 5 |
| 101 | 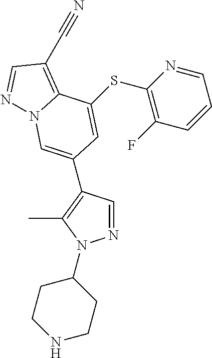 [SMILES: Cc1c(-c2cc(Sc3ncccc3F)c3c(C#N)cnn3c2)cnn1C1CCNCC1] | 4-[(3-fluoro-2-pyridyl) sulfanyl]-6-[5-methyl-1-(4- piperidyl)pyrazol-4-yl] pyrazolo[1,5-a]pyridine-3- carbonitrile | Example 1 | 434.1 | 2 |
| 102 | 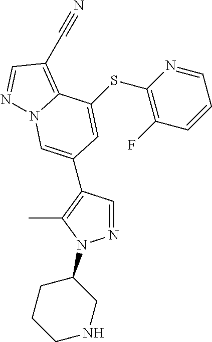 [SMILES: Cc1c(-c2cc(Sc3ncccc3F)c3c(C#N)cnn3c2)cnn1[C@@H]1CCCNC1] | 4-[(3-fluoro-2- pyridyl)sulfanyl]-6-[5- methyl-1-[(3R)-3- piperidyl]pyrazol-4- yl]pyrazolo[1,5-a]pyridine-3- carbonitrile | Example 1 | 434.2 | 6 |
| 103 | 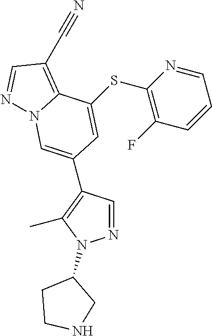 [SMILES: Cc1c(-c2cc(Sc3ncccc3F)c3c(C#N)cnn3c2)cnn1[C@H]1CCNC1] | 4-[(3-fluoro-2- pyridyl)sulfanyl]-6-[5- methyl-1-[(3R)-3- piperidyl]pyrazol-4- yl]pyrazolo[1,5-a]pyridine-3- carbonitrile | Example 1 | 420.0 | 4 |
| 104 | 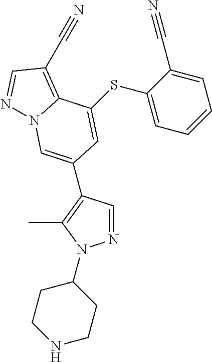 [SMILES: Cc1c(-c2cc(Sc3ccccc3C#N)c3c(C#N)cnn3c2)cnn1C1CCNCC1] | 4-[(3-fluoro-2- pyridyl)sulfanyl]-6-[5- methyl-1-[(3R)-3- piperidyl]pyrazol-4- yl]pyrazolo[1,5-a]pyridine-3- carbonitrile | Example 1 | 440.2 | 1 |
| 105 | 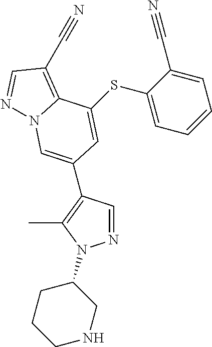 [SMILES: Cc1c(-c2cc(Sc3ccccc3C#N)c3c(C#N)cnn3c2)cnn1[C@H]1CCCNC1] | 4-(2-cyanophenyl)sulfanyl- 6-[5-methyl-1-[(3S)-3- piperidyl]pyrazol-4- yl]pyrazolo[1,5-a]pyridine-3- carbonitrile | Example 1 | 440.1 | 1 |
| 106 | 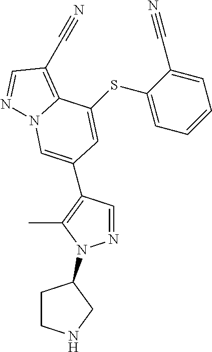 [SMILES: Cc1c(-c2cc(Sc3ccccc3C#N)c3c(C#N)cnn3c2)cnn1[C@@H]1CCNC1] | 4-(2-cyanophenyl) sulfanyl-6-[5-methyl-1- [(3R)-pyrrolidin-3- yl]pyrazol-4-yl]pyrazolo[1,5- a]pyridine-3-carbonitrile | Example 1 | 427.0 | 2 |
| 107 | 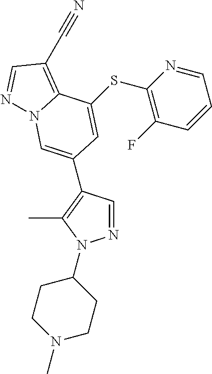 [SMILES: Cc1c(-c2cc(Sc3ncccc3F)c3c(C#N)cnn3c2)cnn1C1CCN(C)CC1] | 4-[(3-fluoro-2- pyridyl)sulfanyl]-6-[5- methyl-1-(1-methyl-4- piperidyl)pyrazol-4- yl]pyrazolo[1,5-a]pyridine3- carbonitrile | Example 1 | 448.1 | 1 |
| 108 | 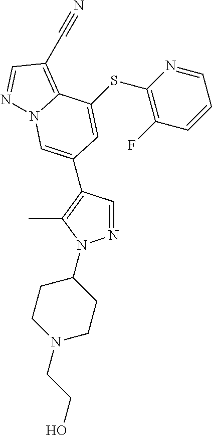 [SMILES: Cc1c(-c2cc(Sc3ncccc3F)c3c(C#N)cnn3c2)cnn1C1CCN(CCO)CC1] | 4-[(3-fluoro-2- pyridyl)sulfanyl]-6-[5- methyl-1-(1-methyl-4- piperidyl)pyrazol-4- yl]pyrazolo[1,5-a]pyridine3- carbonitrilee | Example 3 | 478.0 | 2 |
| 109 | 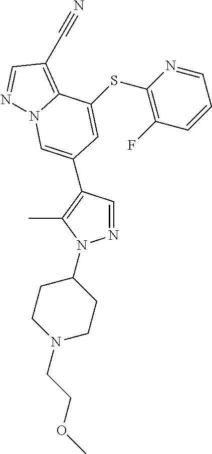 [SMILES: COCCN1CCC(n2ncc(-c3cc(Sc4ncccc4F)c4c(C#N)cnn4c3)c2C)CC1] | 4-[(3-fluoro-2- pyridyl)sulfanyl]-6-[1-[1-(2- methoxyethyl)-4-piperidyl]- 5-methyl-pyrazol-4- yl]pyrazolo[1,5-a]pyridine-3- carbonitrile | Example 35 | 492.0 | 2 |
| 110 | 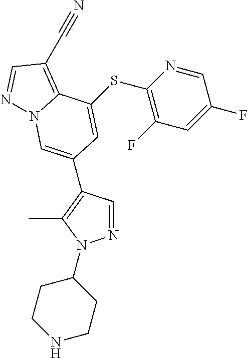 [SMILES: Cc1c(-c2cc(Sc3ncc(F)cc3F)c3c(C#N)cnn3c2)cnn1C1CCNCC1] | 4-[(3,5-difluoro-2- pyridyl)sulfanyl]-6-[5- methyl-1-(4- piperidyl)pyrazol-4- yl]pyrazolo[1,5-a]pyridine-3- carbonitrile | Example 1 | 452.0 | 3 |
| 111 | 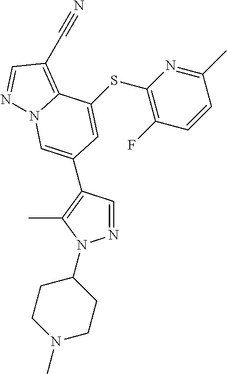 [SMILES: Cc1ccc(F)c(Sc2cc(-c3cnn(C4CCN(C)CC4)c3C)cn3ncc(C#N)c23)n1] | 4-[(3-fluoro-6-methyl-2- pyridyl)sulfanyl]-6-[5- methyl-1-(1-methyl-4- piperidyl)pyrazol-4- yl]pyrazolo[1,5-a]pyridine-3- carbonitrile | Example 1 | 462.1 | 3 |
| 112 | 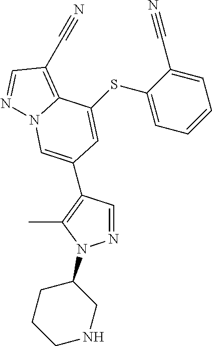 [SMILES: Cc1c(-c2cc(Sc3ccccc3C#N)c3c(C#N)cnn3c2)cnn1[C@@H]1CCCNC1] | 4-(2-cyanophenyl)sulfanyl- 6-[5-methyl-1-[(3R)-3- piperidyl]pyrazol-4- yl]pyrazolo[1,5-a]pyridine-3- carbonitrile | Example 1 | 440.2 | 2 |
| 113 | 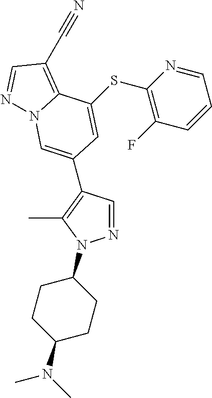 [SMILES: Cc1c(-c2cc(Sc3ncccc3F)c3c(C#N)cnn3c2)cnn1[C@H]1CC[C@@H](N(C)C)CC1] | 4-(2-cyanophenyl)sulfanyl- 6-[5-methyl-1-[(3R)-3- piperidyl]pyrazol-4- yl]pyrazolo[1,5-a]pyridine-3- carbonitrile | Example 1 | 476.2 | 1 |
| 114 | 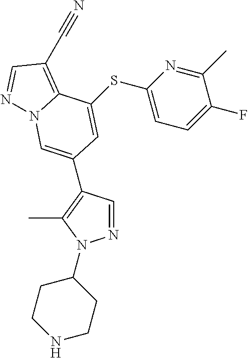 [SMILES: Cc1nc(Sc2cc(-c3cnn(C4CCNCC4)c3C)cn3ncc(C#N)c23)ccc1F] | 4-[(5-fluoro-6-methyl-2- pyridyl)sulfanyl]-6-[5- methyl-1-(4- piperidyl)pyrazol-4- yl]pyrazolo[1,5-a]pyridine-3- carbonitrile | Example 1 | 448.0 | 3 |
| 115 | 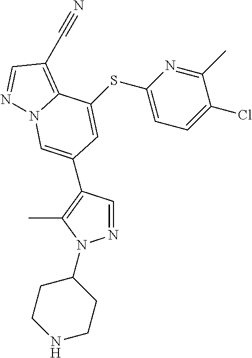 [SMILES: Cc1nc(Sc2cc(-c3cnn(C4CCNCC4)c3C)cn3ncc(C#N)c23)ccc1Cl] | 4-[(5-chloro-6-methyl-2- pyridyl)sulfanyl]-6-[5- methyl-1-(4- piperidyl)pyrazol-4- yl]pyrazolo[1,5-a]pyridine-3- carbonitrile | Example 1 | 464.1 | 1 |
| 116 | 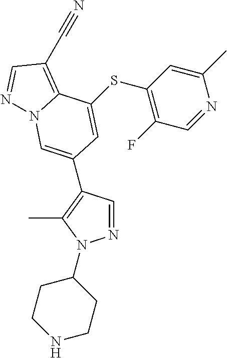 [SMILES: Cc1cc(Sc2cc(-c3cnn(C4CCNCC4)c3C)cn3ncc(C#N)c23)c(F)cn1] | -[(5-fluoro-2-methyl-4- pyridyl)sulfanyl]-6-[5- methyl-1-(4- piperidyl)pyrazol-4- yl]pyrazolo[1,5-a]pyridine-3- carbonitrile | Example 1 | 448.0 | 45 |
| 117 | 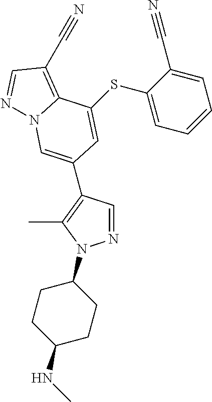 [SMILES: CN[C@H]1CC[C@@H](n2ncc(-c3cc(Sc4ccccc4C#N)c4c(C#N)cnn4c3)c2C)CC1] | -[(5-fluoro-2-methyl-4- pyridyl)sulfany1]-6-[5- methyl-1-(4- piperidyl)pyrazol-4- yl]pyrazolo[1,5-a]pyridine-3- carbonitrile | Example 1 | 468.0 | 0.3 |
| 118 | 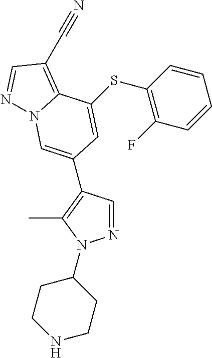 [SMILES: Cc1c(-c2cc(Sc3ccccc3F)c3c(C#N)cnn3c2)cnn1C1CCNCC1] | 4-(2-fluorophenyl)sulfanyl- 6-[5-methyl-1-(4- piperidyl)pyrazol-4- yl]pyrazolo[1,5-a]pyridine-3- carbonitrile | Example 1 | 433.2 | 1 |
| 119 | 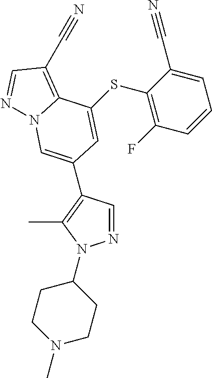 [SMILES: Cc1c(-c2cc(Sc3c(F)cccc3C#N)c3c(C#N)cnn3c2)cnn1C1CCN(C)CC1] | 4-[(3-fluoro-2- pyridyl)sulfanyl]-6-[1-[1-(1- prop-2-enoylazetidine-3- carbonyl)-4- piperidyl]pyrazol-4- yl]pyrazolo[1,5-a]pyridine-3- carbonitrile | Example 1 | 472.1 | 1 |
| 120 | 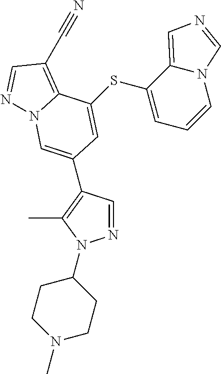 [SMILES: Cc1c(-c2cc(Sc3cccn4cncc34)c3c(C#N)cnn3c2)cnn1C1CCN(C)CC1] | 4-imidazo[1,5-a]pyridin-8- ylsulfany1-6-[5-methyl-1-(1- methyl-4-piperidyl)pyrazol- 4-yl]pyrazolo[1,5-a]pyridine- 3-carbonitrile | Example 1 | 469.1 | 1 |
| 121 | 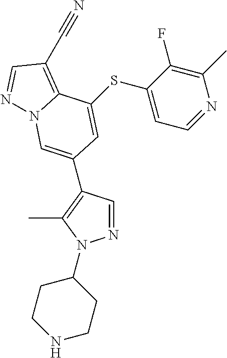 [SMILES: Cc1nccc(Sc2cc(-c3cnn(C4CCNCC4)c3C)cn3ncc(C#N)c23)c1F] | 4-imidazo[1,5-a]pyridin-8- ylsulfanyl-6-[5-methyl-1-(1- methyl-4-piperidyl)pyrazol- 4-yl]pyrazolo[1,5-a]pyridine- 3-carbonitrile | Example 1 | 448.3 | 99 |
| 122 | 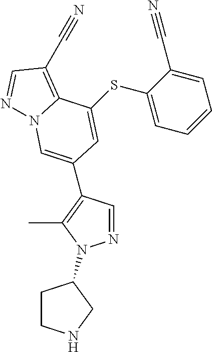 [SMILES: Cc1c(-c2cc(Sc3ccccc3C#N)c3c(C#N)cnn3c2)cnn1[C@H]1CCNC1] | 4-(2-cyanophenyl)sulfanyl- 6-[5-methyl-1-[(3S)- pyrrolidin-3-yl]pyrazol-4- yl]pyrazolo[1,5-a]pyridine-3- carbonitrile | Example 1 | 426.0 | 1 |
| 123 | 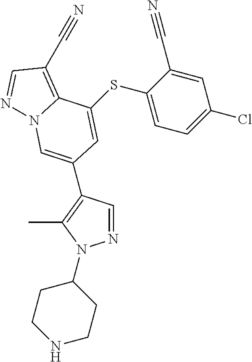 [SMILES: Cc1c(-c2cc(Sc3ccc(Cl)cc3C#N)c3c(C#N)cnn3c2)cnn1C1CCNCC1] | 4-(4-chloro-2-cyano- phenyl)sulfanyl-6-[5-methyl- 1-(4-piperidyl)pyrazol-4- yl]pyrazolo[1,5-a]pyridine-3- carbonitrile | Example 1 | 474.1 | 9 |
| 124 | 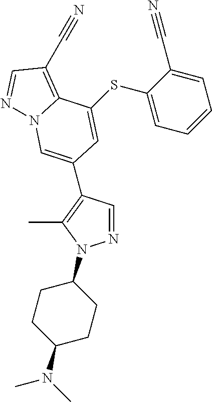 [SMILES: Cc1c(-c2cc(Sc3ccccc3C#N)c3c(C#N)cnn3c2)cnn1[C@H]1CC[C@@H](N(C)C)CC1] | 4-(4-chloro-2-cyano- phenyl)sulfanyl-6-[5-methyl- 1-(4-piperidyl)pyrazol-4- yl]pyrazolo[1,5-a]pyridine-3- carbonitrile | Example 1 | 482.2 | 1 |
| 125 | 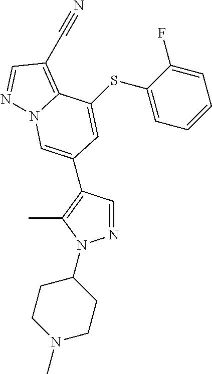 [SMILES: Cc1c(-c2cc(Sc3ccccc3F)c3c(C#N)cnn3c2)cnn1C1CCN(C)CC1] | 4-(4-chloro-2-cyano- phenyl)sulfanyl-6-[5-methyl- 1-(4-piperidyl)pyrazol-4- yl]pyrazolo[1,5-a]pyridine-3- carbonitrile | Example 1 | 447.2 | 1 |
| 126 | 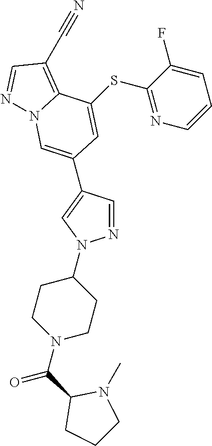 [SMILES: CN1CCC[C@H]1C(=O)N1CCC(n2cc(-c3cc(Sc4ncccc4F)c4c(C#N)cnn4c3)cn2)CC1] | 4-[(3-fluoro-2- pyridyl)sulfanyl]-6-[1-[1- [(2S)-1-methylpyrrolidine-2- carbonyl]-4- piperidyl]pyrazol-4- yl]pyrazolo[1,5-a]pyridine-3- carbonitrile | Example 36 | 531.2 | 1 |
| 127 | 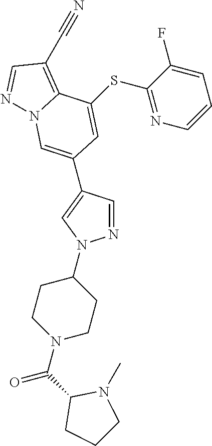 [SMILES: CN1CCC[C@@H]1C(=O)N1CCC(n2cc(-c3cc(Sc4ncccc4F)c4c(C#N)cnn4c3)cn2)CC1] | 4-[(3-fluoro-2- pyridyl)sulfanyl]-6-[1-[1- [(2R)-1-methylpyrrolidine-2- carbonyl]-4- piperidyl]pyrazol-4- yl]pyrazolo[1,5-a]pyridine-3- carbonitrile | Example 1 | 531.2 | 1 |
| 128 | 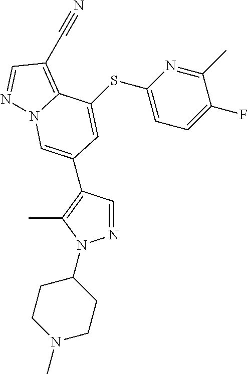 [SMILES: Cc1nc(Sc2cc(-c3cnn(C4CCN(C)CC4)c3C)cn3ncc(C#N)c23)ccc1F] | 4-[(5-fluoro-6-methyl-2- pyridyl)sulfany1]-6-[5- methyl-1-(4- piperidyl)pyrazol-4- yl]pyrazolo[1,5-a]pyridine-3- carbonitrile | Example 1 | 462.0 | 5 |
| 129 | 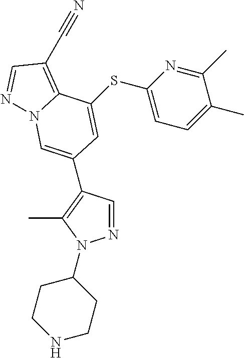 [SMILES: Cc1ccc(Sc2cc(-c3cnn(C4CCNCC4)c3C)cn3ncc(C#N)c23)nc1C] | 4-[(5-fluoro-6-methyl-2- pyridyl)sulfanyl]-6-[5- methyl-1-(4- piperidyl)pyrazol-4- yl]pyrazolo[1,5-a]pyridine-3- carbonitrile | Example 1 | 444.2 | 2 |
| 130 | 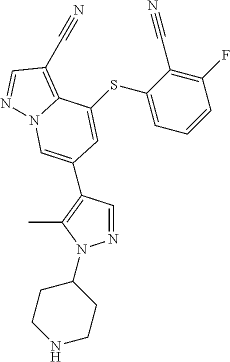 [SMILES: Cc1c(-c2cc(Sc3cccc(F)c3C#N)c3c(C#N)cnn3c2)cnn1C1CCNCC1] | 4-(2-cyano-3-fluoro- phenyl)sulfanyl-6-[5-methyl- 1-(4-piperidyl)pyrazol-4- yl]pyrazolo[1,5-a]pyridine- 3-carbonitrile | Example 1 | 458.1 | 2 |
| 131 | 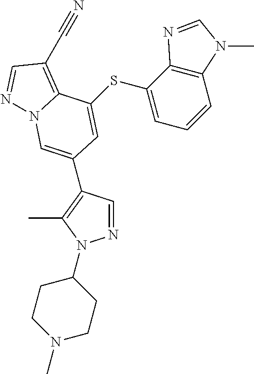 [SMILES: Cc1c(-c2cc(Sc3cccc4c3ncn4C)c3c(C#N)cnn3c2)cnn1C1CCN(C)CC1] | 4-(1-methylbenzimidazol-4- yl)sulfanyl-6-[5-methyl-1-(1- methyl-4-piperidyl)pyrazol- 4-yl]pyrazolo[1,5-a]pyridine- 3-carbonitrile | Example 1 | 483.3 | 40 |
| 132 | 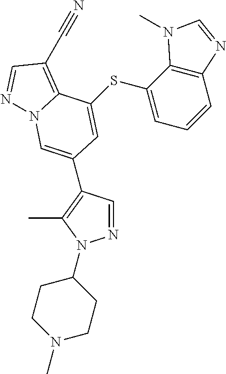 [SMILES: Cc1c(-c2cc(Sc3cccc4ncn(C)c34)c3c(C#N)cnn3c2)cnn1C1CCN(C)CC1] | 4-(3-methylbenzimidazol-4- yl)sulfanyl-6-[5-methyl-1-(1- methyl-4-piperidyl)pyrazol- 4-yl]pyrazolo[1,5-a]pyridine- 3-carbonitrile | Example 1 | 483.2 | 10 |
| 133 | 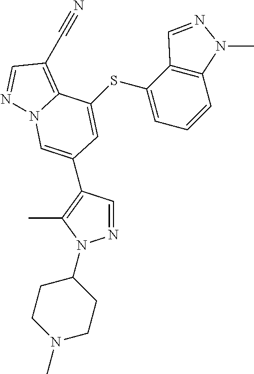 [SMILES: Cc1c(-c2cc(Sc3cccc4c3cnn4C)c3c(C#N)cnn3c2)cnn1C1CCN(C)CC1] | 4-(1-methylindazol-4- yl)sulfanyl-6-[5-methyl-1-(1- methyl-4-piperidyl)pyrazol- 4-yl]pyrazolo[1,5-a]pyridine- 3-carbonitrile | Example 1 | 483.1 | 6 |
| 134 | 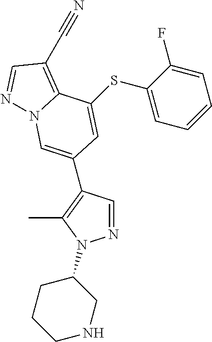 [SMILES: Cc1c(-c2cc(Sc3ccccc3F)c3c(C#N)cnn3c2)cnn1[C@H]1CCCNC1] | 4-(2-fluorophenyl)sulfanyl- 6-[5-methyl-1-[(3S)-3- piperidyl]pyrazol-4- yl]pyrazolo[1,5-a]pyridine-3- carbonitrile | Example 1 | 433.1 | 1 |

TABLE 4
A comparison of activation of the FGFR in the presence of concentration,
in nM among compound IDs 1, 117, 128, and 130.

| | | | FGFR2 | FGFR2 | FGFR2 |
|---|---|---|---|---|---|
| Example | FGFR2 | FGFR2 | N549H | V564F | V564I |
| Number | Enz | wt cell | Enzyme | Enzyme | Enzyme |
| Example 1 | 3 | 25 | 1.5 | 3 | 6 |
| Example 117 | 0.3 | 28 | 0.2 | 0.5 | 1 |
| Example 128 | 0.12 | 41 | 4 | 9 | 12 |
| Example 130 | 1 | 117 | 3 | 4 | 5 |

| | | | | MS | Enz |
|---|---|---|---|---|---|
| Compound | | | Reference | APCI | FGFR2 |
| number | Structure | Chemical Name | Procedure | (m/z) | (nM) |
| 135 | 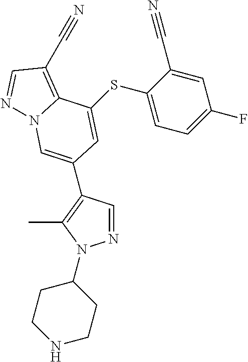 [SMILES: Cc1c(-c2cc(Sc3ccc(F)cc3C#N)c3c(C#N)cnn3c2)cnn1C1CCNCC1] | 4-4-((2-cyano-4- fluorophenyl)thio)-6-(5- methyl-1-(piperidin-4-yl)- 1H-pyrazol-4- yl)pyrazolo[1,5- a]pyridine-3-carbonitrile | Example 110 | 458.2.0 | 1 |
| 136 | 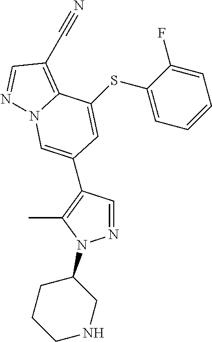 [SMILES: Cc1c(-c2cc(Sc3ccccc3F)c3c(C#N)cnn3c2)cnn1[C@@H]1CCCNC1] | (R)-4-((2- fluorophenyl)thio)-6-(5- methyl-1-(piperidin-3-yl)- 1H-pyrazol-4- yl)pyrazolo[1,5- a]pyridine-3-carbonitrile | Example 1 | 433.1 | 4 |
| 137 | 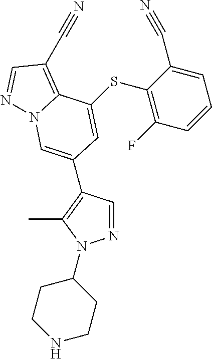 [SMILES: Cc1c(-c2cc(Sc3c(F)cccc3C#N)c3c(C#N)cnn3c2)cnn1C1CCNCC1] | 4-((2-cyano-6- fluorophenyl)thio)-6-(5- methyl-1-(piperidin-4-yl)- 1H-pyrazol-4- yl)pyrazolo[1,5- a]pyridine-3-carbonitrile | Example 10 | 458.0 | 1 |
| 138 | 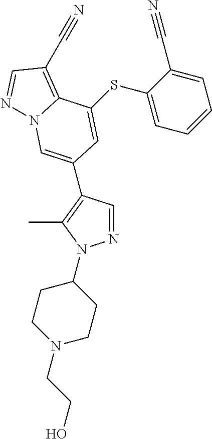 [SMILES: Cc1c(-c2cc(Sc3ccccc3C#N)c3c(C#N)cnn3c2)cnn1C1CCN(CCO)CC1] | 4-((2-cyanophenyl)thio)- 6-(1-(1-(2- hydroxyethyl)piperidin-4- yl)-5-methyl-1H-pyrazol- 4-yl)pyrazolo[1,5- a]pyridine-3-carbonitrile | Example 35 | 484.2 | 1 |
| 139 | 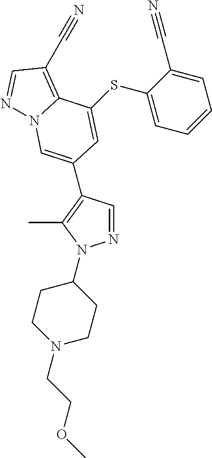 [SMILES: COCCN1CCC(n2ncc(-c3cc(Sc4ccccc4C#N)c4c(C#N)cnn4c3)c2C)CC1] | 4-((2-cyanophenyl)thio)- 6-(1-(1-(2- methoxyethyl)piperidin-4- yl)-5-methyl-1H-pyrazol- 4-yl)pyrazolo[1,5- a]pyridine-3-carbonitrile | Example 35 | 498.1 | 1 |
| 140 | 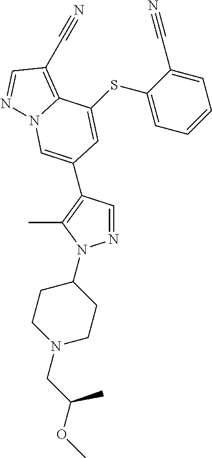 [SMILES: CO[C@H](C)CN1CCC(n2ncc(-c3cc(Sc4ccccc4C#N)c4c(C#N)cnn4c3)c2C)CC1] | (R)-4-((2- cyanophenyl)thio)-6-(1- (1-(2- methoxypropyl)piperidin- 4-yl)-5-methyl-1H- pyrazol-4-yl)pyrazolo[1,5- a]pyridine-3-carbonitrile | Eaxmple 35 | 512.1 | 1 |
| 141 | 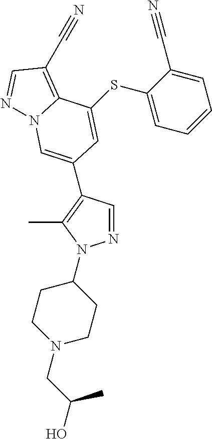 [SMILES: Cc1c(-c2cc(Sc3ccccc3C#N)c3c(C#N)cnn3c2)cnn1C1CCN(C[C@@H](C)O)CC1] | (R)-4-((2- cyanophenyl)thio)-6-(1- (1-(2- hydroxypropyl)piperidin- 4-yl)-5-methyl-1H- pyrazol-4-yl)pyrazolo[1,5- a]pyridine-3-carbonitrile | Example 35 | 498.2 | 1 |
| 142 | 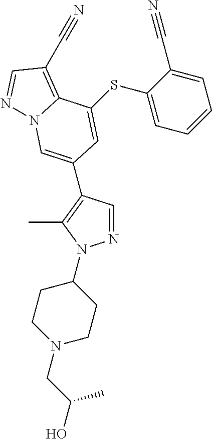 [SMILES: Cc1c(-c2cc(Sc3ccccc3C#N)c3c(C#N)cnn3c2)cnn1C1CCN(C[C@H](C)O)CC1] | (S)-4-((2- cyanophenyl)thio)-6-(1- (1-(2- hydroxypropyl)piperidin- 4-yl)-5-methyl-1H- pyrazol-4-yl)pyrazolo[1,5- a]pyridine-3-carbonitrile | Example 35 | 498.1 | 13 |
| 143 | 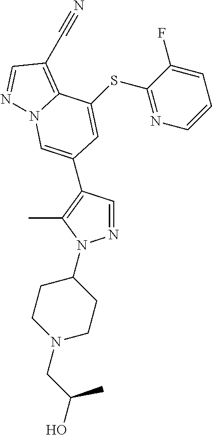 [SMILES: Cc1c(-c2cc(Sc3ncccc3F)c3c(C#N)cnn3c2)cnn1C1CCN(C[C@@H](C)O)CC1] | (R)-4-((3-fluoropyridin-2- yl)thio)-6-(1-(1-(2- hydroxypropyl)piperidin- 4-yl)-5-methyl-1H- pyrazol-4-yl)pyrazolo[1,5- a]pyridine-3-carbonitrile | Example 35 | 492.2 | 2 |
| 144 | 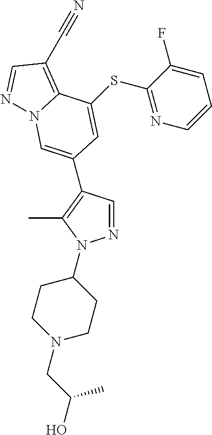 [SMILES: Cc1c(-c2cc(Sc3ncccc3F)c3c(C#N)cnn3c2)cnn1C1CCN(C[C@H](C)O)CC1] | (S)-4-((3-fluoropyridin-2- yl)thio)-6-(1-(1-(2- hydroxypropyl)piperidin- 4-yl)-5-methyl-1H- pyrazol-4-yl)pyrazolo[1,5- a]pyridine-3-carbonitrile | Example 35 | 492.1 | 1.5 |
| 145 | 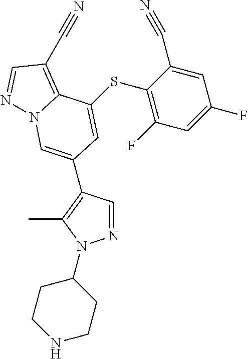 [SMILES: Cc1c(-c2cc(Sc3c(F)cc(F)cc3C#N)c3c(C#N)cnn3c2)cnn1C1CCNCC1] | 4-((2-cyano-4,6- difluorophenyl)thio)-6-(5- methyl-1-(piperidin-4-yl)- 1H-pyrazol-4- yl)pyrazolo[1,5- a]pyridine-3-carbonitrile | Example 10 | 476.0 | 7 |
| 146 | 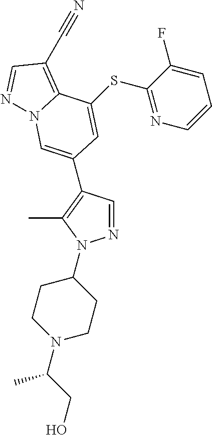 [SMILES: Cc1c(-c2cc(Sc3ncccc3F)c3c(C#N)cnn3c2)cnn1C1CCN([C@@H](C)CO)CC1] | (S)-4-((3-fluoropyridin-2- yl)thio)-6-(1-(1-(1- hydroxypropan-2- yl)piperidin-4-yl)-5- methyl-1H-pyrazol-4- yl)pyrazolo[1,5- a]pyridine-3-carbonitrile | Example 35 | 492.3 | 2 |
| 147 | 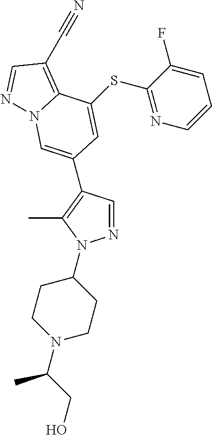 [SMILES: Cc1c(-c2cc(Sc3ncccc3F)c3c(C#N)cnn3c2)cnn1C1CCN([C@H](C)CO)CC1] | (R)-4-((3-fluoropyridin-2- yl)thio)-6-(1-(1-(1- hydroxypropan-2- yl)piperidin-4-yl)-5- methyl-1H-pyrazol-4- yl)pyrazolo[1,5- a]pyridine-3-carbonitrile | Example 35 | 492.3 | 2 |
| 148 | 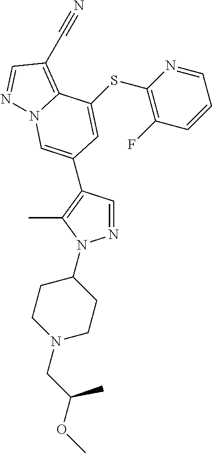 [SMILES: CO[C@H](C)CN1CCC(n2ncc(-c3cc(Sc4ncccc4F)c4c(C#N)cnn4c3)c2C)CC1] | (R)-4-((3-fluoropyridin-2- yl)thio)-6-(1-(1-(2- methoxypropyl)piperidin- 4-yl)-5-methyl-1H- pyrazol-4-yl)pyrazolo[1,5- a]pyridine-3-carbonitrile | Example 35 | 506.2 | 2 |
| 149 | 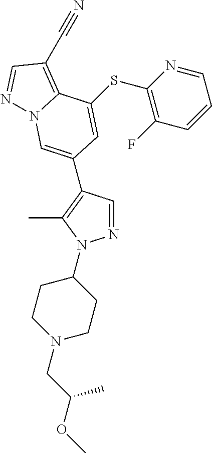 [SMILES: CO[C@@H](C)CN1CCC(n2ncc(-c3cc(Sc4ncccc4F)c4c(C#N)cnn4c3)c2C)CC1] | (S)-4-((3-fluoropyridin-2- yl)thio)-6-(1-(1-(2- methoxypropyl)piperidin- 4-yl)-5-methyl-1H- pyrazol-4-yl)pyrazolo[1,5- a]pyridine-3-carbonitrile | Example 35 | 506.4 | 2 |
| 150 | 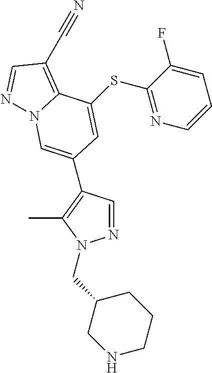 [SMILES: Cc1c(-c2cc(Sc3ncccc3F)c3c(C#N)cnn3c2)cnn1C[C@@H]1CCCNC1] | (R)-4-((3-fluoropyridin-2- yl)thio)-6-(5-methyl-1- (piperidin-3-ylmethyl)- 1H-pyrazol-4- yl)pyrazolo[1,5- a]pyridine-3-carbonitrile | Example 1 | 453.1 | 4 |
| 151 | 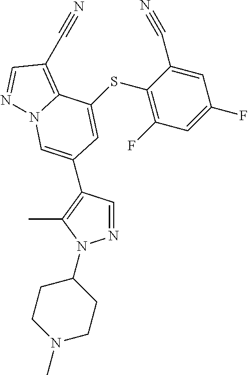 [SMILES: Cc1c(-c2cc(Sc3c(F)cc(F)cc3C#N)c3c(C#N)cnn3c2)cnn1C1CCN(C)CC1] | 4-((2-cyano-4,6- difluorophenyl)thio)-6-(5- methyl-1-(1- methylpiperidin-4-yl)-1H- pyrazol-4-yl)pyrazolo[1,5- a]pyridine-3- carbonitrileyl)pyrazolo[1, 5-a]pyridine-3-carbonitrile | Example 1 | 490.1 | 2 |
| 152 | 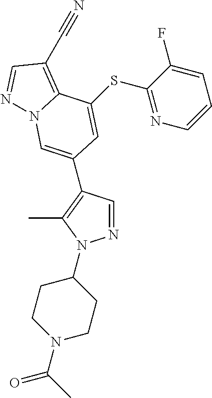 [SMILES: CC(=O)N1CCC(n2ncc(-c3cc(Sc4ncccc4F)c4c(C#N)cnn4c3)c2C)CC1] | 6-(1-(1-acetylpiperidin-4- yl)-5-methyl-1H-pyrazol- 4-yl)-4-((3-fluoropyridin- 2-yl)thio)pyrazolo[1,5- a]pyridine-3-carbonitrile | Example 36 | 476.1 | 2 |
| 153 | 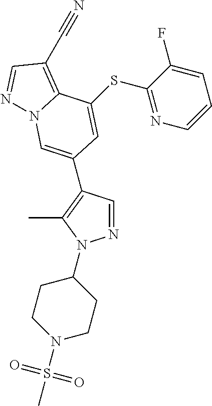 [SMILES: Cc1c(-c2cc(Sc3ncccc3F)c3c(C#N)cnn3c2)cnn1C1CCN(S(C)(=O)=O)CC1] | 4-((3-fluoropyridin-2- yl)thio)-6-(5-methyl-1-(1- (methylsulfonyl)piperidin- 4-yl)-1H-pyrazol-4- yl)pyrazolo[1,5- a]pyridine-3-carbonitrile | | 512.1 | 2 |
| 154 | 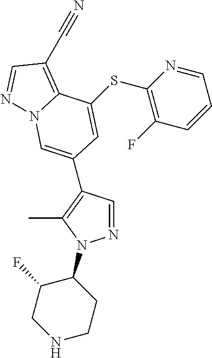 [SMILES: Cc1c(-c2cc(Sc3ncccc3F)c3c(C#N)cnn3c2)cnn1[C@H]1CCNC[C@@H]1F] | 6-(1-((3S,4S)-3- fluoropiperidin-4-yl)-5- methyl-1H-pyrazol-4-yl)- 4-((3-fluoropyridin-2- yl)thio)pyrazolo[1,5- a]pyridine-3-carbonitrile | | 453.1 | 2 |
| 155 | 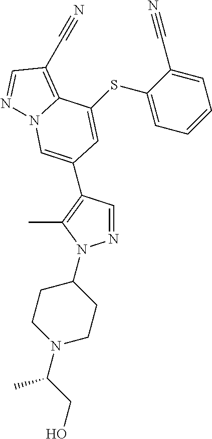 [SMILES: Cc1c(-c2cc(Sc3ccccc3C#N)c3c(C#N)cnn3c2)cnn1C1CCN([C@@H](C)CO)CC1] | (S)-4-((2- cyanophenyl)thio)-6-(1- (1-(1-hydroxypropan-2- yl)piperidin-4-yl)-5- methyl-1H-pyrazol-4- yl)pyrazolo[1,5- a]pyridine-3-carbonitirle | Example 35 | 498.2 | 1 |
| 156 | 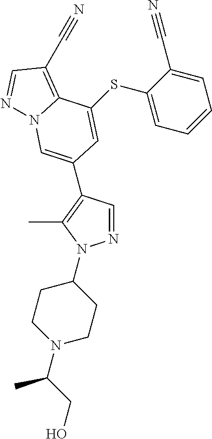 [SMILES: Cc1c(-c2cc(Sc3ccccc3C#N)c3c(C#N)cnn3c2)cnn1C1CCN([C@H](C)CO)CC1] | (R)-4-((2- cyanophenyl)thio)-6-(1- (1-(1-hydroxypropan-2- yl)piperidin-4-yl)-5- methyl-1H-pyrazol-4- yl)pyrazolo[1,5- a]pyridine-3-carbonitrile | Example 35 | 498.4 | 1 |
| 157 | 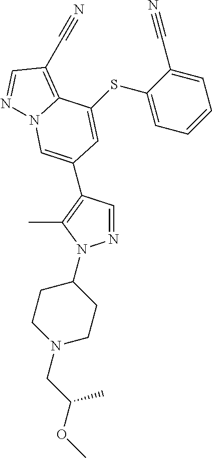 [SMILES: CO[C@@H](C)CN1CCC(n2ncc(-c3cc(Sc4ccccc4C#N)c4c(C#N)cnn4c3)c2C)CC1] | (S)-4-((2- cyanophenyl)thio)-6-(1- (1-(2- methoxypropyl)piperidin- 4-yl)-5-methyl-1H- pyrazol-4-yl)pyrazolo[1,5- a]pyridine-3-carbonitrile | Example 35 | 512.2 | 1 |
| 158 | 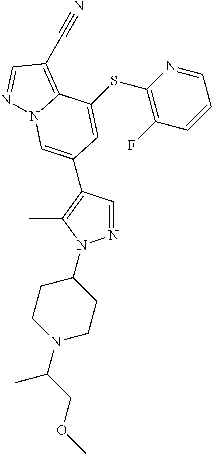 [SMILES: COCC(C)N1CCC(n2ncc(-c3cc(Sc4ncccc4F)c4c(C#N)cnn4c3)c2C)CC1] | 4-((3-fluoropyridin-2- yl)thio)-6-(1-(1-(1- methoxypropan-2- yl)piperidin-4-yl)-5- methyl-1H-pyrazol-4- yl)pyrazolo[1,5- a]pyridine-3-carbonitrile | Example 35 | 506.4 | 2 |
| 159 | 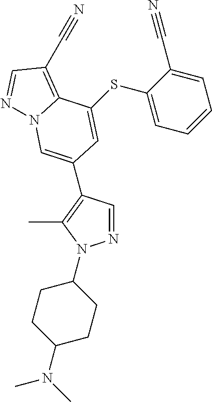 [SMILES: Cc1c(-c2cc(Sc3ccccc3C#N)c3c(C#N)cnn3c2)cnn1C1CCC(N(C)C)CC1] | 4-((2-cyanophenyl)thio)- 6-(1-(4- (dimethylamino)cyclohex- yl)-5-methyl-1H-pyrazol- 4-yl)pyrazolo[1,5- a]pyridine-3-carbonitrile | Example 1 | 482.3 | 0.5 |
| 160 | 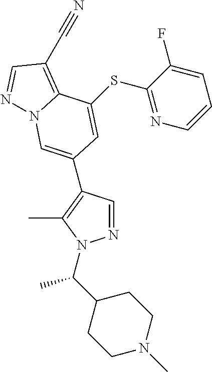 [SMILES: Cc1c(-c2cc(Sc3ncccc3F)c3c(C#N)cnn3c2)cnn1[C@@H](C)C1CCN(C)CC1] | (S)-4-((3-fluoropyridin-2- yl)thio)-6-(5-methyl-1-(1- (1-methylpiperidin-4- yl)ethyl)-1H-pyrazol-4- yl)pyrazolo[1,5- a]pyridine-3-carbonitrile | Example 32 | 476.3 | 2 |
| 161 | 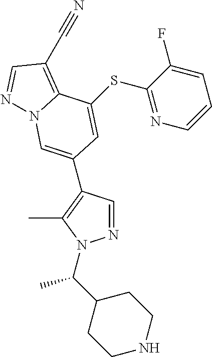 [SMILES: Cc1c(-c2cc(Sc3ncccc3F)c3c(C#N)cnn3c2)cnn1[C@@H](C)C1CCNCC1] | (S)-4-((3-fluoropyridin-2- yl)thio)-6-(5-methyl-1-(1- (piperidin-4-yl)ethyl)-1H- pyrazol-4-yl)pyrazolo[1,5- a]pyridine-3-carbonitrile | | 462.3 | 2 |
| 162 | 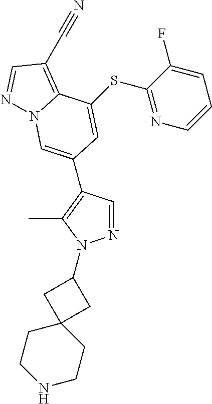 [SMILES: Cc1c(-c2cc(Sc3ncccc3F)c3c(C#N)cnn3c2)cnn1C1CC2(CCNCC2)C1] | 4-((3-fluoropyridin-2- yl)thio)-6-(5-methyl-1-(7- azaspiro[3.5]nonan-2-yl)- 1H-pyrazol-4- yl)pyrazolo[1,5- a]pyridine-3-carbonitrile | Example 52 | 474.2 | 1 |
| 163 | 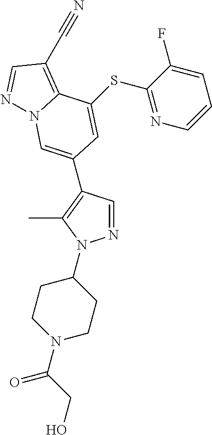 [SMILES: Cc1c(-c2cc(Sc3ncccc3F)c3c(C#N)cnn3c2)cnn1C1CCN(C(=O)CO)CC1] | 4-((3-fluoropyridin-2- yl)thio)-6-(1-(1-(2- hydroxyacetyl)piperidin- 4-yl)-5-methyl-1H- pyrazol-4-yl)pyrazolo[1,5- a]pyridine-3-carbonitrile | Example 36 | 492.1 | 3 |
| 164 | 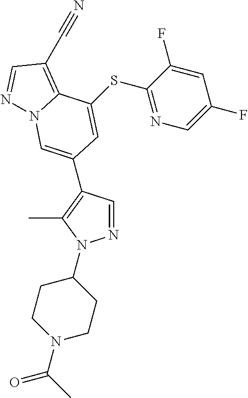 [SMILES: CC(=O)N1CCC(n2ncc(-c3cc(Sc4ncc(F)cc4F)c4c(C#N)cnn4c3)c2C)CC1] | 6-(1-(1-acetylpiperidin-4- yl)-5-methyl-1H-pyrazol- 4-yl)-4-((3,5- difluoropyridin-2- yl)thio)pyrazolo[1,5- a]pyridine-3-carbonitrile | Example 36 | 494.2 | 5 |
| 165 | 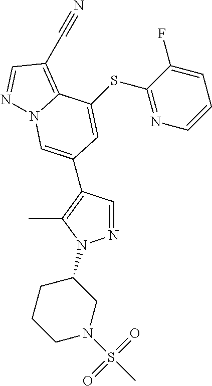 [SMILES: Cc1c(-c2cc(Sc3ncccc3F)c3c(C#N)cnn3c2)cnn1[C@H]1CCCN(S(C)(=O)=O)C1] | (S)-4-((3-fluoropyridin-2- yl)thio)-6-(5-methyl-1-(1- (methylsulfonyl)piperidin- 3-yl)-1H-pyrazol-4- yl)pyrazolo[1,5- a]pyridine-3-carbonitrile | Example 50 | 512.1 | 2 |
| 166 | 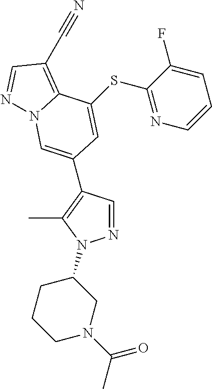 [SMILES: CC(=O)N1CCC[C@H](n2ncc(-c3cc(Sc4ncccc4F)c4c(C#N)cnn4c3)c2C)C1] | (S)-6-(1-(1- acetylpiperidin-3-yl)-5- methyl-1H-pyrazol-4-yl)- 4-((3-fluoropyridin-2- yl)thio)pyrazolo[1,5- a]pyridine-3-carbonitrile | Example 30 | 476.2 | 2 |
| 167 | 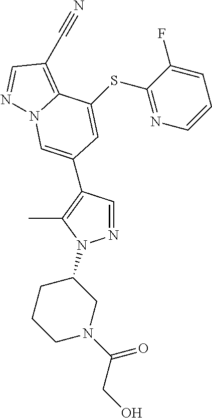 [SMILES: Cc1c(-c2cc(Sc3ncccc3F)c3c(C#N)cnn3c2)cnn1[C@H]1CCCN(C(=O)CO)C1] | (S)-4-((3-fluoropyridin-2- yl)thio)-6-(1-(1-(2- hydroxyacetyl)piperidin- 3-yl)-5-methyl-1H- pyrazol-4-yl)pyrazolo[1,5- a]pyridine-3-carbonitrile | Example 30 | 492.0 | 2 |
| 168 | 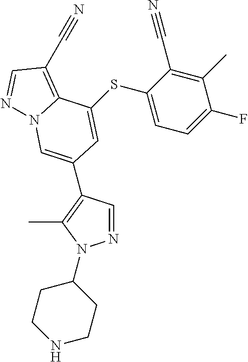 [SMILES: Cc1c(F)ccc(Sc2cc(-c3cnn(C4CCNCC4)c3C)cn3ncc(C#N)c23)c1C#N] | 4-((1-cyano-4-fluoro-3- methylphenyl)thio)-6-(5- methyl-1-(piperidin-4-yl)- 1H-pyrazol-4- yl)pyrazolo[1,5- a]pyridine-3-carbonitrile | Example 1 | 472.4 | 1 |
| 169 | 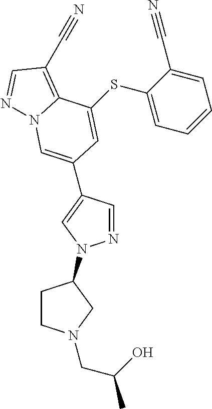 [SMILES: C[C@H](O)CN1CC[C@@H](n2cc(-c3cc(Sc4ccccc4C#N)c4c(C#N)cnn4c3)cn2)C1] | 4-((2-cyanophenyl)thio)- 6-(1-((R)-1-((S)-2- hydroxypropyl)pyrrolidin- 3-yl)-1H-pyrazol-4- yl)pyrazolo[1,5- a]pyridine-3-carbonitrile | Example 57 | 470.2 | 0.4 |
| 170 | 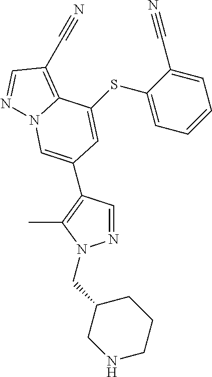 [SMILES: Cc1c(-c2cc(Sc3ccccc3C#N)c3c(C#N)cnn3c2)cnn1C[C@@H]1CCCNC1] | (R)-4-((2- cyanophenyl)thio)-6-(5- methyl-1-(piperidin-3- ylmethyl)-1H-pyrazol-4- yl)pyrazolo[1,5- a]pyridine-3-carbonitrile | | 454.1 | 72 |
| 171 | 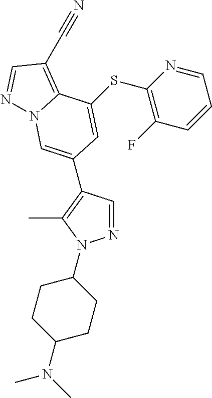 [SMILES: Cc1c(-c2cc(Sc3ncccc3F)c3c(C#N)cnn3c2)cnn1C1CCC(N(C)C)CC1] | 6-(1-(4- (dimethylamino)cyclohex- yl)-5-methyl-1H-pyrazol- 4-yl)-4-((3-fluoropyridin- 2-yl)thio)pyrazolo[1,5- a]pyridine-3-carbonitrile | Example 1 | 476.2 | 1 |
| 172 | 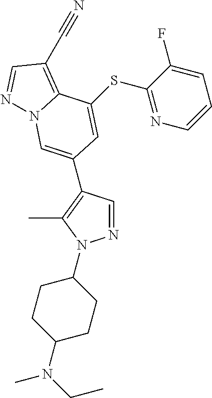 [SMILES: CCN(C)C1CCC(n2ncc(-c3cc(Sc4ncccc4F)c4c(C#N)cnn4c3)c2C)CC1] | 6-(1-(4- (ethyl(methyl)amino)cyclo- hexyl)-5-methyl-1H- pyrazol-4-yl)-4-((3- fluoropyridin-2- yl)thio)pyrazolo[1,5- a]pyridine-3-carbonitrile | Example 32 | 490.2 | 1 |
| 173 | 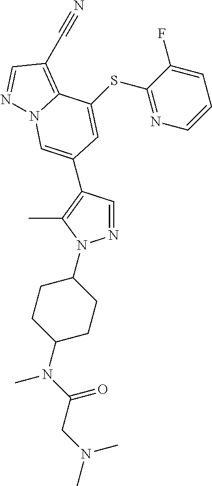 [SMILES: Cc1c(-c2cc(Sc3ncccc3F)c3c(C#N)cnn3c2)cnn1C1CCC(N(C)C(=O)CN(C)C)CC1] | N-(4-(4-(3-cyano-4-((3- fluoropyridin-2- yl)thio)pyrazolo[1,5- a]pyridin-6-yl)-5-methyl- 1H-pyrazol-1- yl)cyclohexyl)-2- (dimethylamino)-N- methylacetamide | Example 36 | 547.4 | 1 |
| 174 | 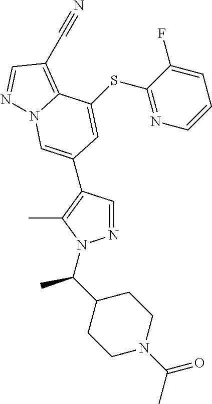 [SMILES: CC(=O)N1CCC([C@@H](C)n2ncc(-c3cc(Sc4ncccc4F)c4c(C#N)cnn4c3)c2C)CC1] | (R)-6-(1-(1-(1- acetylpiperidin-4- yl)ethyl)-5-methyl-1H- pyrazol-4-yl)-4-((3- fluoropyridin-2- yl)thio)pyrazolo[1,5- a]pyridine-3-carbonitrile | Example 39 | 504.4 | 4 |
| 175 | 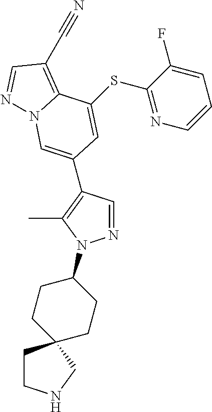 [SMILES: Cc1c(-c2cc(Sc3ncccc3F)c3c(C#N)cnn3c2)cnn1[C@H]1CC[C@@]2(CCNC2)CC1] | 4-((3-fluoropyridin-2- yl)thio)-6-(5-methyl-1- ((5r,8r)-2- azaspiro[4.5]decan-8-yl)- 1H-pyrazol-4- yl)pyrazolo[1,5- a]pyridine-3-carbonitrile | Example 53 | 488.2 | 1 |
| 176 | 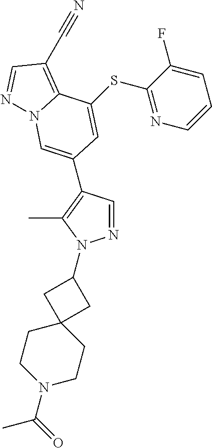 [SMILES: CC(=O)N1CCC2(CC1)CC(n1ncc(-c3cc(Sc4ncccc4F)c4c(C#N)cnn4c3)c1C)C2] | 6-(1-(7-acetyl-7- azaspiro[3.5]nonan-2-yl)- 5-methyl-1H-pyrazol-4- yl)-4-((3-fluoropyridin-2- yl)thio)pyrazolo[1,5- a]pyridine-3-carbonitrile | Example 39 | 516.3 | 1.5 |
| 177 | 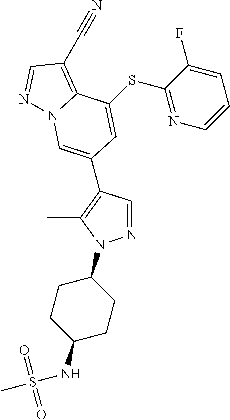 [SMILES: Cc1c(-c2cc(Sc3ncccc3F)c3c(C#N)cnn3c2)cnn1[C@H]1CC[C@@H](NS(C)(=O)=O)CC1] | N-((1s,4s)-4-(4-(3-cyano- 4-((3-fluoropyridin-2- yl)thio)pyrazolo[1,5- a]pyridin-6-yl)-5-methyl- 1H-pyrazol-1- yl)cyclohexyl)methane- sulfonamide | Example 53 | 526.3 | 1 |
| 178 | 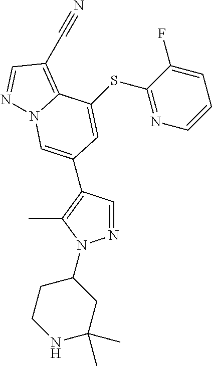 [SMILES: Cc1c(-c2cc(Sc3ncccc3F)c3c(C#N)cnn3c2)cnn1C1CCNC(C)(C)C1] | 6-(1-(7-acetyl-7- azaspiro[3.5]nonan-2-yl)- 5-methyl-1H-pyrazol-4- yl)-4-((3-fluoropyridin-2- yl)thio)pyrazolo[1,5- a]pyridine-3-carbonitrile | Example 53 | 462.3 | 1 |
| 179 | 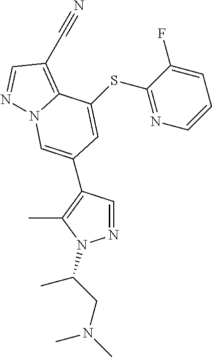 [SMILES: Cc1c(-c2cc(Sc3ncccc3F)c3c(C#N)cnn3c2)cnn1[C@@H](C)CN(C)C] | (S)-6-(1-(1- (dimethylamino)propan-2- yl)-5-methyl-1H-pyrazol- 4-yl)-4-((3-fluoropyridin- 2-yl)thio)pyrazolo[1,5- a]pyridine-3-carbonitrile | Example 53 | 436.2 | 3 |
| 180 | 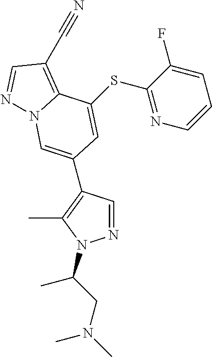 [SMILES: Cc1c(-c2cc(Sc3ncccc3F)c3c(C#N)cnn3c2)cnn1[C@H](C)CN(C)C] | (R)-6-(1-(1- (dimethylamino)propan-2- yl)-5-methyl-1H-pyrazol- 4-yl)-4-((3-fluoropyridin- 2-yl)thio)pyrazolo[1,5- a]pyridine-3-carbonitrile | Example 53 | 436.2 | 15 |
| 181 | 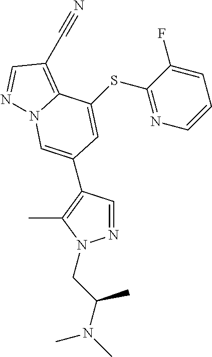 [SMILES: Cc1c(-c2cc(Sc3ncccc3F)c3c(C#N)cnn3c2)cnn1C[C@@H](C)N(C)C] | (R)-6-(1-(2- (dimethylamino)propyl)- 5-methyl-1H-pyrazol-4- yl)-4-((3-fluoropyridin-2- yl)thio)pyrazolo[1,5- a]pyridine-3-carbonitrile | Example 53 | 436.3 | 7 |
| 182 | 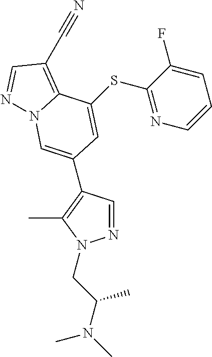 [SMILES: Cc1c(-c2cc(Sc3ncccc3F)c3c(C#N)cnn3c2)cnn1C[C@H](C)N(C)C] | (S)-6-(1-(2- (dimethylamino)propyl)- 5-methyl-1H-pyrazol-4- yl)-4-((3-fluoropyridin-2- yl)thio)pyrazolo[1,5- a]pyridine-3-carbonitrile | Example 53 | 436.2 | 8 |
| 183 | 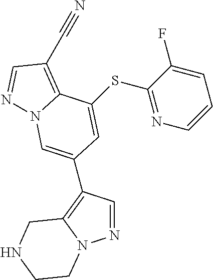 [SMILES: N#Cc1cnn2cc(-c3cnn4c3CNCC4)cc(Sc3ncccc3F)c12] | 4-((3-fluoropyridin-2- yl)thio)-6-(4,5,6,7- tetrahydropyrazolo[1,5- a]pyrazin-3- yl)pyrazolo[1,5- a]pyridine-3-carbonitrile | Example 53 | 392.2 | 8 |
| 184 | 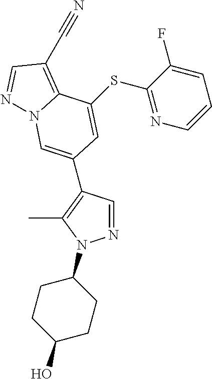 [SMILES: Cc1c(-c2cc(Sc3ncccc3F)c3c(C#N)cnn3c2)cnn1[C@H]1CC[C@@H](O)CC1] | 4-((3-fluoropyridin-2- yl)thio)-6-(1-((1s,4s)-4- hydroxycyclohexyl)-5- methyl-1H-pyrazol-4- yl)pyrazolo[1,5- a]pyridine-3-carbonitrile | Example 53 | 449.1 | 1.6 |
| 185 | 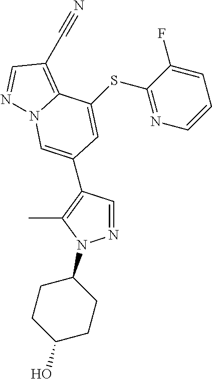 [SMILES: Cc1c(-c2cc(Sc3ncccc3F)c3c(C#N)cnn3c2)cnn1[C@H]1CC[C@H](O)CC1] | 4-((3-fluoropyridin-2- yl)thio)-6-(1-((1r,4r)-4- hydroxycyclohexyl)-5- methyl-1H-pyrazol-4- yl)pyrazolo[1,5- a]pyridine-3-carbonitrile | Example 53 | 449.1 | 1 |
| 186 | 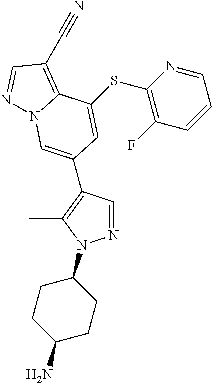 [SMILES: Cc1c(-c2cc(Sc3ncccc3F)c3c(C#N)cnn3c2)cnn1[C@H]1CC[C@@H](N)CC1] | 6-(1-((1s,4s)-4- aminocyclohexyl)-5- methyl-1H-pyrazol-4-yl)- 4-((3-fluoropyridin-2- yl)thio)pyrazolo[1,5- a]pyridine-3-carbonitrile | Example 22 | 448.3 | 3 |
| 187 | 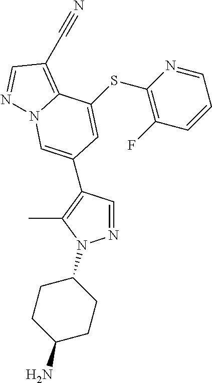 [SMILES: Cc1c(-c2cc(Sc3ncccc3F)c3c(C#N)cnn3c2)cnn1[C@H]1CC[C@H](N)CC1] | 6-(1-((1r,4r)-4- aminocyclohexyl)-5- methyl-1H-pyrazol-4-yl)- 4-((3-fluoropyridin-2- yl)thio)pyrazolo[1,5- a]pyridine-3-carbonitrile | Example 22 | 448.1 | 1 |
| 188 | 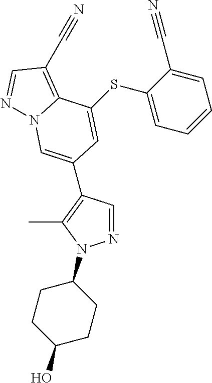 [SMILES: Cc1c(-c2cc(Sc3ccccc3C#N)c3c(C#N)cnn3c2)cnn1[C@H]1CC[C@@H](O)CC1] | 4-((2-cyanophenyl)thio)- 6-(1-((1s,4s)-4- hydroxycyclohexyl)-5- methyl-1H-pyrazol-4- yl)pyrazolo[1,5- a]pyridine-3-carbonitrile | Example 53 | 455.2 | 0.5 |
| 189 | 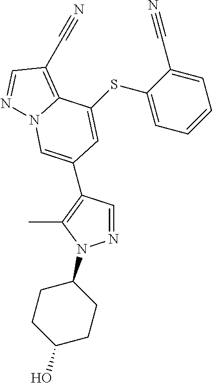 [SMILES: Cc1c(-c2cc(Sc3ccccc3C#N)c3c(C#N)cnn3c2)cnn1[C@H]1CC[C@H](O)CC1] | 4-((2-cyanophenyl)thio)- 6-(1-((1r,4r)-4- hydroxycyclohexyl)-5- methyl-1H-pyrazol-4- yl)pyrazolo[1,5- a]pyridine-3-carbonitrile | Example 53 | 455.2 | 0.7 |
| 190 | 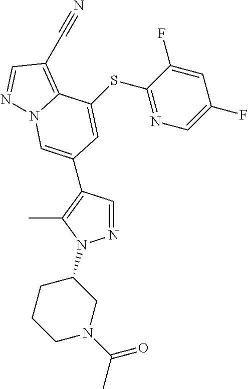 [SMILES: CC(=O)N1CCC[C@H](n2ncc(-c3cc(Sc4ncc(F)cc4F)c4c(C#N)cnn4c3)c2C)C1] | (S)-6-(1-(1- acetylpiperidin-3-yl)-5- methyl-1H-pyrazol-4-yl)- 4-((3,5-difluoropyridin-2- yl)thio)pyrazolo[1,5- a]pyridine-3-carbonitrile | Example 30 | 494.1 | 3 |
| 191 | 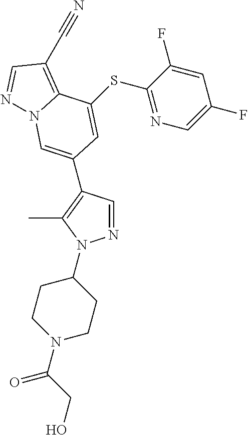 [SMILES: Cc1c(-c2cc(Sc3ncc(F)cc3F)c3c(C#N)cnn3c2)cnn1C1CCN(C(=O)CO)CC1] | 4-((3,5-difluoropyridin-2- yl)thio)-6-(1-(1-(2- hydroxyacetyl)piperidin- 4-yl)-5-methyl-1H- pyrazol-4-yl)pyrazolo[1,5- a]pyridine-3-carbonitrile | Example 36 | 510.2 | 4 |
| 192 | 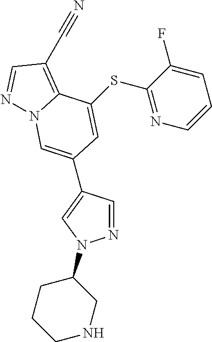 [SMILES: N#Cc1cnn2cc(-c3cnn([C@@H]4CCCNC4)c3)cc(Sc3ncccc3F)c12] | (R)-4-((3-fluoropyridin-2- yl)thio)-6-(1-(piperidin-3- yl)-1H-pyrazol-4- yl)pyrazolo[1,5- a]pyridine-3-carbonitrile | Example 1 | 420.2 | 2 |
| 193 | 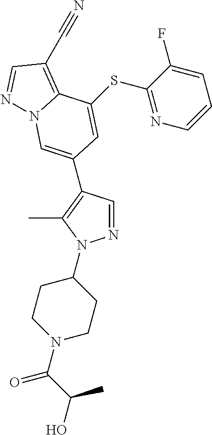 [SMILES: Cc1c(-c2cc(Sc3ncccc3F)c3c(C#N)cnn3c2)cnn1C1CCN(C(=O)[C@@H](C)O)CC1] | (R)-4-((3-fluoropyridin-2- yl)thio)-6-(1-(1-(2- hydroxypropanoyl)piperidin- 4-yl)-5-methyl-1H- pyrazol-4-yl)pyrazolo[1,5- a]pyridine-3-carbonitrile | Example 36 | 506.4 | 2 |
| 194 | 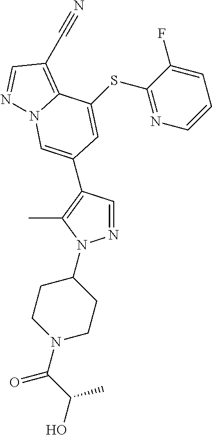 [SMILES: Cc1c(-c2cc(Sc3ncccc3F)c3c(C#N)cnn3c2)cnn1C1CCN(C(=O)[C@H](C)O)CC1] | (S)-4-((3-fluoropyridin-2- yl)thio)-6-(1-(1-(2- hydroxypropanoyl)piperidin- 4-yl)-5-methyl-1H- pyrazol-4-yl)pyrazolo[1,5- a]pyridine-3-carbonitirle | Example 36 | 506.2 | 2 |
| 195 | 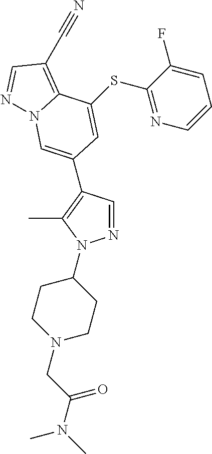 [SMILES: Cc1c(-c2cc(Sc3ncccc3F)c3c(C#N)cnn3c2)cnn1C1CCN(CC(=O)N(C)C)CC1] | 2-(4-(4-(3-cyano-4-((3- fluoropyridin-2- yl)thio)pyrazolo[1,5- a]pyridin-6-yl)-5-methyl- 1H-pyrazol-1- yl)piperidin-1-yl)-N,N- dimethylacetamide | Example 35 | 519.3 | 1.4 |
| 196 | 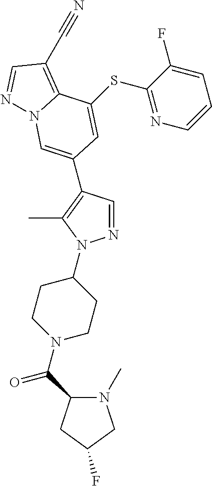 [SMILES: Cc1c(-c2cc(Sc3ncccc3F)c3c(C#N)cnn3c2)cnn1C1CCN(C(=O)[C@@H]2C[C@@H](F)CN2C)CC1] | 6-(1-(1-((2S,4R)-4-fluoro- 1-methylpyrrolidine-2- carbonyl)piperidin-4-yl)- 5-methyl-1H-pyrazol-4- yl)-4-((3-fluoropyridin-2- yl)thio)pyrazolo[1,5- a]pyridine-3-carbonitrile | Example 36 | 563.4 | 1 |
| 197 | 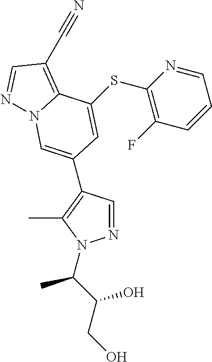 [SMILES: Cc1c(-c2cc(Sc3ncccc3F)c3c(C#N)cnn3c2)cnn1[C@H](C)[C@H](O)CO] | 6-(1-((2R,3S)-3,4- dihydroxybutan-2-yl)-5- methyl-1H-pyrazol-4-yl)- 4-((3-fluoropyridin-2- yl)thio)pyrazolo[1,5- a]pyridine-3-carbonitrile | Example 53 | 439.3 | 3 |
| 198 | 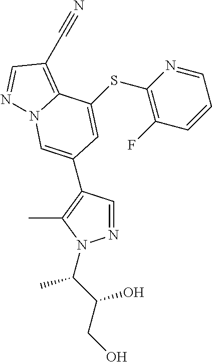 [SMILES: Cc1c(-c2cc(Sc3ncccc3F)c3c(C#N)cnn3c2)cnn1[C@@H](C)[C@H](O)CO] | 6-(1-((2S,3S)-3,4- dihydroxybutan-2-yl)-5- methyl-1H-pyrazol-4-yl)- 4-((3-fluoropyridin-2- yl)thio)pyrazolo[1,5- a]pyridine-3-carbonitrile | Example 53 | 439.3 | 21 |
| 199 | 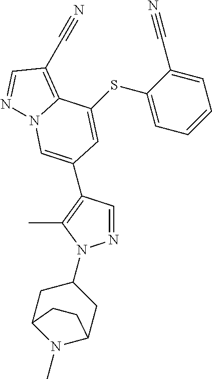 [SMILES: Cc1c(-c2cc(Sc3ccccc3C#N)c3c(C#N)cnn3c2)cnn1C1CC2CCC(C1)N2C] | 4-((2-cyanophenyl)thio)- 6-(5-methyl-1-(8-methyl- 8-azabicyclo[3.2.1]octan- 3-yl)-1H-pyrazol-4 yl)pyrazolo[1,5- a]pyridine-3-carbonitrile | Example 53 | 480.3 | 1 |
| 200 | 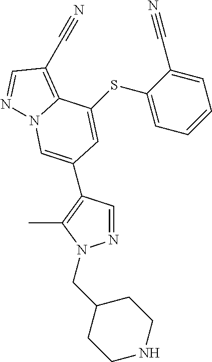 [SMILES: Cc1c(-c2cc(Sc3ccccc3C#N)c3c(C#N)cnn3c2)cnn1CC1CCNCC1] | 4-((2-cyanophenyl)thio)- 6-(5-methyl-1-(piperidin- 4-ylmethyl)-1H-pyrazol- 4-yl)pyrazolo[1,5- a]pyridine-3-carbonitrile | Example 53 | 454.3 | 1 |
| 201 | 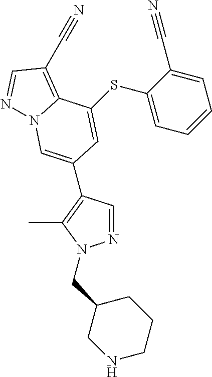 [SMILES: Cc1c(-c2cc(Sc3ccccc3C#N)c3c(C#N)cnn3c2)cnn1C[C@H]1CCCNC1] | (S)-4-((2- cyanophenyl)thio)-6-(5- methyl-1-(piperidin-3- ylmethyl)-1H-pyrazol-4- yl)pyrazolo[1,5- a]pyridine-3-carbonitrile | Example 53 | 454.3 | 2 |
| 202 | 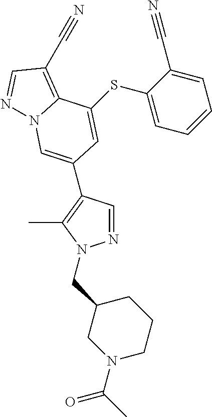 [SMILES: CC(=O)N1CCC[C@H](Cn2ncc(-c3cc(Sc4ccccc4C#N)c4c(C#N)cnn4c3)c2C)C1] | (S)-6-(1-((1- acetylpiperidin-3- yl)methyl)-5-methyl-1H- pyrazol-4-yl)-4-((2- cyanophenyl)thio)pyrazolo [1,5-a]pyridine-3- carbonitrile | Example 39 | 496.2 | 2 |
| 203 | 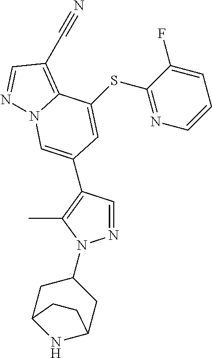 [SMILES: Cc1c(-c2cc(Sc3ncccc3F)c3c(C#N)cnn3c2)cnn1C1CC2CCC(C1)N2] | 4-[(3-fluoro-2- pyridiyl)sulfonyl]-6-[1-[1- (1-prop-2-enoylazetidine- 3-carbonyl)-4- piperidyl]pyrazol-4- yl]pyrazolo[1,5- a]pyridine-3-carbonitrile | Example 53 | 460.3 | 2 |
| 204 | 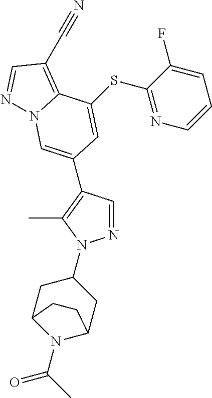 [SMILES: CC(=O)N1C2CCC1CC(n1ncc(-c3cc(Sc4ncccc4F)c4c(C#N)cnn4c3)c1C)C2] | 6-(1-(8-acetyl-8- azabicyclo[3.2.1]octan-3- yl)-5-methyl-1H-pyrazol- 4-yl)-4-((3-fluoropyridin- 2-yl)thio)pyrazolo[1,5- a]pyridine-3-carbonitrile | Example 39 | 502.3 | 2 |
| 205 | 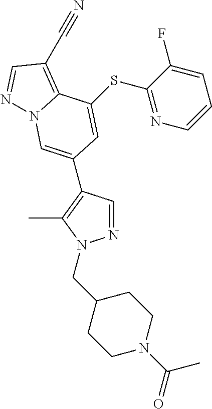 [SMILES: CC(=O)N1CCC(Cn2ncc(-c3cc(Sc4ncccc4F)c4c(C#N)cnn4c3)c2C)CC1] | 6-(1-((1-acetylpiperidin-4- yl)methyl)-5-methyl-1H- pyrazol-4-yl)-4-((3- fluoropyridin-2- yl)thio)pyrazolo[1,5- a]pyridine-3-carbonitrile | Example 39 | 490.2 | 3 |
| 206 | 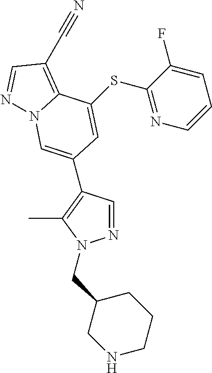 [SMILES: Cc1c(-c2cc(Sc3ncccc3F)c3c(C#N)cnn3c2)cnn1C[C@H]1CCCNC1] | (S)-4-((3-fluoropyridin-2- yl)thio)-6-(5-methyl-1- (piperidin-3-ylmethyl)- 1H-pyrazol-4- yl)pyrazolo[1,5- a]pyridine-3-carbonitrile | Example 53 | 448.3 | 3 |
| 207 | 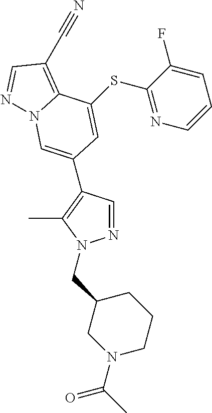 [SMILES: CC(=O)N1CCC[C@H](Cn2ncc(-c3cc(Sc4ncccc4F)c4c(C#N)cnn4c3)c2C)C1] | (S)-6-(1-((1- acetylpiperidin-3- yl)methyl)-5-methyl-1H- pyrazol-4-yl)-4-((3- fluoropyridin-2- yl)thio)pyrazolo[1,5- a]pyridine-3-carbonitrile | Example 39 | 490.3 | 5 |
| 208 | 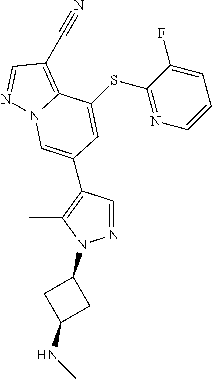 [SMILES: CN[C@H]1C[C@@H](n2ncc(-c3cc(Sc4ncccc4F)c4c(C#N)cnn4c3)c2C)C1] | 4-((3-fluoropyridin-2- yl)thio)-6-(5-methyl-1- ((1s,3s)-3- (methylamino)cyclobutyl)- 1H-pyrazol-4- yl)pyrazolo[1,5- a]pyridine-3-carbonitrile | Example 53 | 434.3 | 1 |
| 209 | 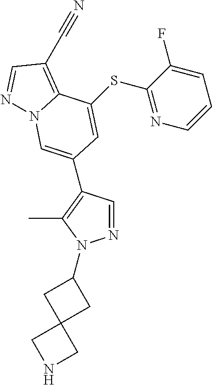 [SMILES: Cc1c(-c2cc(Sc3ncccc3F)c3c(C#N)cnn3c2)cnn1C1CC2(CNC2)C1] | 4-((3-fluoropyridin-2- yl)thio)-6-(5-methyl-1-(2- azaspiro[3.3]heptan-6-yl)- 1H-pyrazol-4- yl)pyrazolo[1,5- a]pyridine-3-carbonitrile | Example 53 | 446.2 | 1 |
| 210 | 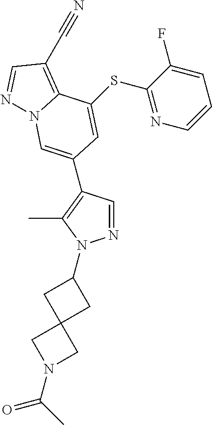 [SMILES: CC(=O)N1CC2(CC(n3ncc(-c4cc(Sc5ncccc5F)c5c(C#N)cnn5c4)c3C)C2)C1] | 6-(1-(2-acetyl-2- azaspiro[3.3]heptan-6-yl)- 5-methyl-1H-pyrazol-4- yl)-4-((3-fluoropyridin-2- yl)thio)pyrazolo[1,5- a]pyridine-3-carbonitrile | Example 39 | 488.1 | 2 |
| 211 | 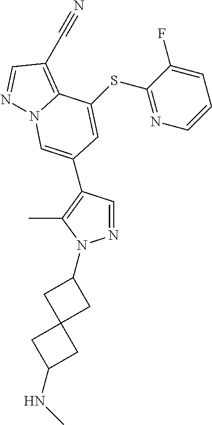 [SMILES: CNC1CC2(C1)CC(n1ncc(-c3cc(Sc4ncccc4F)c4c(C#N)cnn4c3)c1C)C2] | 4-((3-fluoropyridin-2- yl)thio)-6-(5-methyl-1-(6- (methylamino)spiro[3.3]hep- tan-2-yl)-1H-pyrazol-4- yl)pyrazolo[1,5- a]pyridine-3-carbonitrile | Example 53 | 474.2 | 1 |
| 212 | 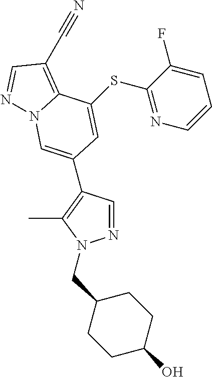 [SMILES: Cc1c(-c2cc(Sc3ncccc3F)c3c(C#N)cnn3c2)cnn1C[C@H]1CC[C@@H](O)CC1] | 4-((3-fluoropyridin-2- yl)thio)-6-(1-(((1s,4s)-4- hydroxycyclohexyl)methyl)- 5-methyl-1H-pyrazol-4- yl)pyrazolo[1,5- a]pyridine-3-carbonitrile | Example 53 | 463.2 | 3 |
| 213 | 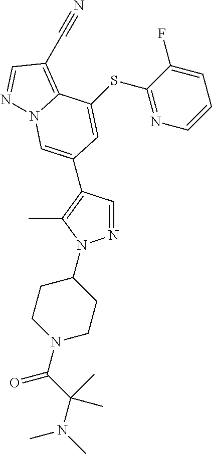 [SMILES: Cc1c(-c2cc(Sc3ncccc3F)c3c(C#N)cnn3c2)cnn1C1CCN(C(=O)C(C)(C)N(C)C)CC1] | 6-(1-(1-(2- (dimethylamino)-2- methylpropanoyl)piperidin- 4-yl)-5-methyl-1H- pyrazol-4-yl)-4-((3- fluoropyridin-2- yl)thio)pyrazolo[1,5- a]pyridine-3-carbonitrile | Example 36 | 547.4 | 1.6 |
| 214 | 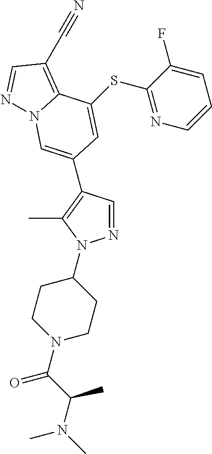 [SMILES: Cc1c(-c2cc(Sc3ncccc3F)c3c(C#N)cnn3c2)cnn1C1CCN(C(=O)[C@@H](C)N(C)C)CC1] | 6-(1-(1-(dimethyl-D- alanyl)piperidin-4-yl)-5- methyl-1H-pyrazol-4-yl)- 4-((3-fluoropyridin-2- yl)thio)pyrazolo[1,5- a]pyridine-3-carbonitrile | Example 36 | 533.2 | 1.6 |
| 215 | 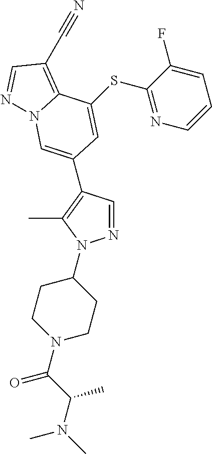 [SMILES: Cc1c(-c2cc(Sc3ncccc3F)c3c(C#N)cnn3c2)cnn1C1CCN(C(=O)[C@H](C)N(C)C)CC1] | 6-(1-(1-(dimethyl-L- alanyl)piperidin-4-yl)-5- methyl-1H-pyrazol-4-yl)- 4-((3-fluoropyridin-2- yl)thio)pyrazolo[1,5- a]pyridine-3-carbonitrile | Example 36 | 533.4 | 1.6 |
| 216 | 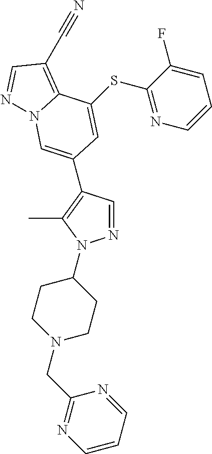 [SMILES: Cc1c(-c2cc(Sc3ncccc3F)c3c(C#N)cnn3c2)cnn1C1CCN(Cc2ncccn2)CC1] | 4-((3-fluoropyridin-2- yl)thio)-6-(5-methyl-1-(1- (pyrimidin-2- ylmethyl)piperidin-4-yl)- 1H-pyrazol-4- yl)pyrazolo[1,5- a]pyridine-3-carbonitrile | Example 35 | 526.4 | 2 |
| 217 | 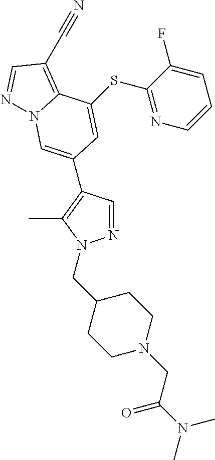 [SMILES: Cc1c(-c2cc(Sc3ncccc3F)c3c(C#N)cnn3c2)cnn1CC1CCN(CC(=O)N(C)C)CC1] | 2-(4-((4-(3-cyano-4-((3- fluoropyridin- 2-yl)thio)pyrazolo[1,5- a]pyridin-6-yl)-5-methyl- 1H-pyrazol-1- yl)methyl)piperidin-1-yl)- N,N-dimethylacetamide | Example 39 | 533.4 | 6 |
| 218 | 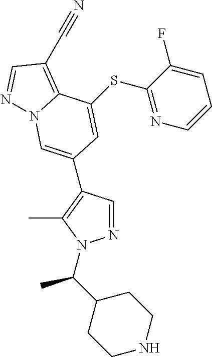 [SMILES: Cc1c(-c2cc(Sc3ncccc3F)c3c(C#N)cnn3c2)cnn1[C@H](C)C1CCNCC1] | (R)-4-((3-fluoropyridin-2- yl)thio)-6-(5-methyl-1-(1- (piperidin-4-yl)ethyl)-1H- pyrazol-4-yl)pyrazolo[1,5- a]pyridine-3-carbonitrile | Example 53 | 462.3 | 3 |
| 219 | 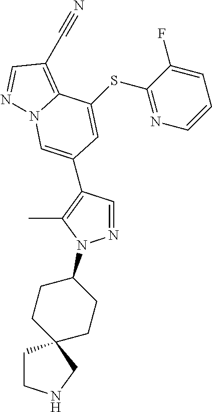 [SMILES: Cc1c(-c2cc(Sc3ncccc3F)c3c(C#N)cnn3c2)cnn1[C@H]1CC[C@]2(CCNC2)CC1] | 4-((3-fluoropyridin-2- yl)thio)-6-(5-methyl-1- ((5s,8s)-2- azaspiro[3.4]decan-8-yl)- 1H-pyrazol-4- yl)pyrazolo[1,5- a]pyridine-3-carbonitrile | Example 53 | 488.2 | 0.5 |
| 220 | 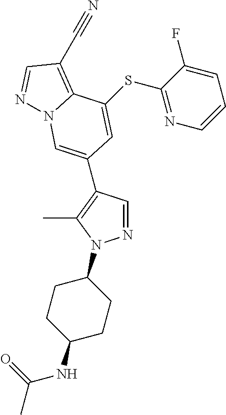 [SMILES: CC(=O)N[C@H]1CC[C@@H](n2ncc(-c3cc(Sc4ncccc4F)c4c(C#N)cnn4c3)c2C)CC1] | N-((1s,4S)-4-(4-(3-cyano- 4-((3-fluoropyridin-2- yl)thio)pyrazolo[1,5- a]pyridin-6-yl)-5-methyl- 1H-pyrazol-1- yl)cyclohexyl)acetamide | Example 39 | 490.3 | 5 |
| 221 | 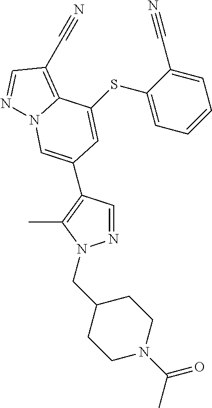 [SMILES: CC(=O)N1CCC(Cn2ncc(-c3cc(Sc4ccccc4C#N)c4c(C#N)cnn4c3)c2C)CC1] | 6-(1-((1-acetylpiperidin-4- yl)methyl)-5-methyl-1H- pyrazol-4-yl)-4-((2- cyanophenyl)thio)pyrazolo [1,5-a]pyridin-3- carbonitrile | Example 39 | 496.4 | 1 |
| 222 | 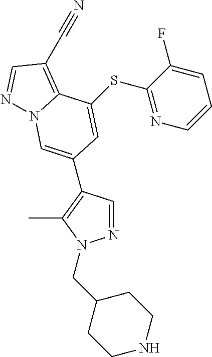 [SMILES: Cc1c(-c2cc(Sc3ncccc3F)c3c(C#N)cnn3c2)cnn1CC1CCNCC1] | 4-((3-fluoropyridin-2- yl)thio)-6-(5-methyl-1- (piperidin-4-ylmethyl)- 1H-pyrazol-4- yl)pyrazolo[1,5- a]pyridine-3-carbonitrile | Example 53 | 448.3 | 5 |
| 223 | 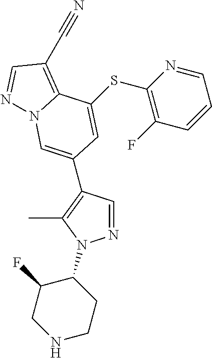 [SMILES: Cc1c(-c2cc(Sc3ncccc3F)c3c(C#N)cnn3c2)cnn1[C@@H]1CCNC[C@H]1F] | 6-(1-((3R,4R)-3- fluoropiperidin-4-yl)-5- methyl-1H-pyrazol-4-yl)- 4-((3-fluoropyridin-2- yl)thio)pyrazolo∥,5- a]pyridine-3-carbonitrile | Example 53 | 452.3 | 8 |
| 224 | 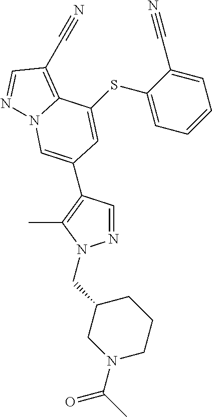 [SMILES: CC(=O)N1CCC[C@@H](Cn2ncc(-c3cc(Sc4ccccc4C#N)c4c(C#N)cnn4c3)c2C)C1] | (R)-6-(1-((1- acetylpiperidin-3- yl)methyl)-5-methyl-1H- pyrazol-4-yl)-4-((2- cyanophenyl)thio)pyrazolo [1,5-a]pyridine-3- carbonitrile | Example 39 | 496.2 | 8 |
| 225 | 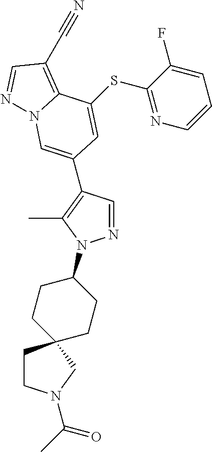 [SMILES: CC(=O)N1CC[C@]2(CC[C@@H](n3ncc(-c4cc(Sc5ncccc5F)c5c(C#N)cnn5c4)c3C)CC2)C1] | 6-(1-((5r,8r)-2-acetyl-2- azaspiro[4.5]decan-8-yl)- 5-methyl-1H-pyrazol-4- y)-4-((3-fluoropyridin-2- yl)thio)pyrazolo[1,5- a]pyridine-3-carbonitrile | Example 39 | 530.4 | 1 |
| 226 | 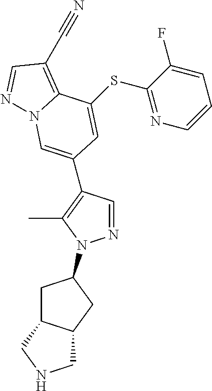 [SMILES: Cc1c(-c2cc(Sc3ncccc3F)c3c(C#N)cnn3c2)cnn1[C@@H]1C[C@H]2CNC[C@H]2C1] | 4-((3-fluoropyridin-2- yl)thio)-6-(5-methyl-1- ((3sR,5s,6aS)- octahydrocyclopenta[c]pyr- rol-5-yl)-1H-pyrazol-4- yl)pyrazolo[1,5- a]pyridine-3-carbonitrile | Example 53 | 460.2 | 1 |
| 227 | 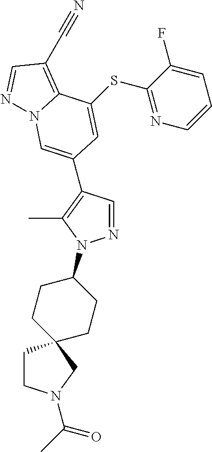 [SMILES: CC(=O)N1CC[C@]2(CC[C@H](n3ncc(-c4cc(Sc5ncccc5F)c5c(C#N)cnn5c4)c3C)CC2)C1] | 6-(1-((5s,8s)-2-acetyl-2- azaspiro[4.5]decan-8-yl)- 5-methyl-1H-pyrazol-4- yl)-4-((3-fluoropyridin-2- yl)thio)pyrazolo[1,5- a]pyridine-3-carbonitrile | Example 39 | 530.2 | 1 |
| 228 | 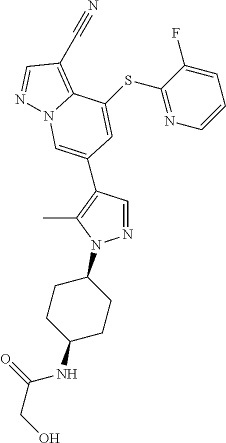 [SMILES: Cc1c(-c2cc(Sc3ncccc3F)c3c(C#N)cnn3c2)cnn1[C@H]1CC[C@@H](NC(=O)CO)CC1] | N-((1s,4s)-4-(4-(3-cyano- 4-((3-fluoropyridin-2- yl)thio)pyrazolo[1,5- a]pyridin-6-yl)-5-methyl- 1H-pyrazol-1- yl)cyclohexyl)-2- hydroxyacetamide | Example 36 | 506.1 | 1 |
| 229 | 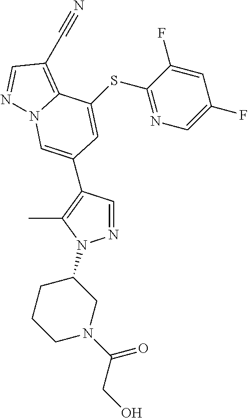 [SMILES: Cc1c(-c2cc(Sc3ncc(F)cc3F)c3c(C#N)cnn3c2)cnn1[C@H]1CCCN(C(=O)CO)C1] | (S)-4-((3,5- difluoropyridin-2-yl)thio)- 6-(1-(1-(2- hydroxyacetyl)piperidin- 3-yl)-5-methyl-1H- pyrazol-4-yl)pyrazolo[1,5- a]pyridine-3-carbonitrile | Example 30 | 510.2 | 3 |
| 230 | 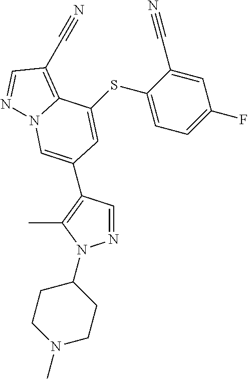 [SMILES: Cc1c(-c2cc(Sc3ccc(F)cc3C#N)c3c(C#N)cnn3c2)cnn1C1CCN(C)CC1] | 4-((2-cyano-4- fluorophenyl)thio)-6-(5- methyl-1-(1- methylpiperidin-4-yl)-1H- pyrazol-4-yl)pyrazolo[1,5- a]pyridine-3-carbonitrile | Example 10 | 472.2 | 1 |
| 231 | 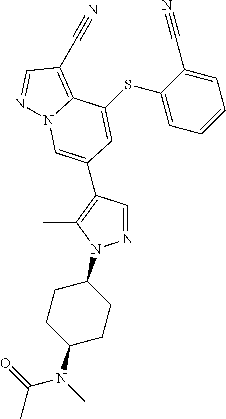 [SMILES: CC(=O)N(C)[C@H]1CC[C@@H](n2ncc(-c3cc(Sc4ccccc4C#N)c4c(C#N)cnn4c3)c2C)CC1] | N-((1s,4s)-4-(4-(3-cyano- 4-((2- cyanophenyl)thio)pyrazolo [1,5-a]pyridin-6-yl)-5- methyl-1H-pyrazol-1- yl)cyclohexyl)-N- methylacetamide | Example 39 | 510.3 | 1 |
| 232 | 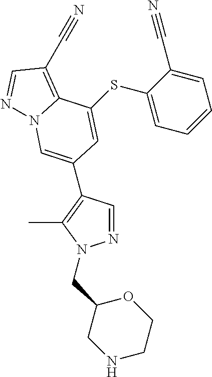 [SMILES: Cc1c(-c2cc(Sc3ccccc3C#N)c3c(C#N)cnn3c2)cnn1C[C@@H]1CNCCO1] | (S)-4-((2- cyanophenyl)thio)-6-(5- methyl-1-(morpholin-2- ylmethyl)-1H-pyrazol-4- yl)pyrazolo[1,5- a]pyridine-3-carbonitrile | Example 53 | 456.3 | 3.5 |
| 233 | 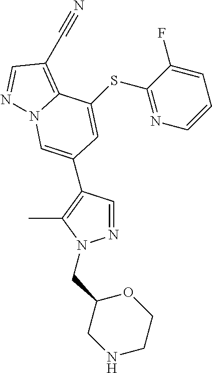 [SMILES: Cc1c(-c2cc(Sc3ncccc3F)c3c(C#N)cnn3c2)cnn1C[C@@H]1CNCCO1] | (S)-4-((3-fluoropyridin-2- yl)thio)-6-(5-methyl-1- (morpholin-2-ylmethyl)- 1H-pyrazol-4- yl)pyrazolo[1,5- a]pyridine-3-carbonitrile | Example 53 | 450.3 | 6 |
| 234 | 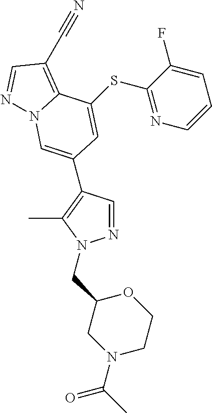 [SMILES: CC(=O)N1CCO[C@H](Cn2ncc(-c3cc(Sc4ncccc4F)c4c(C#N)cnn4c3)c2C)C1] | (S)-6-(1-((4- acetylmorpholin-2- yl)methyl)-5-methyl-1H- pyrazol-4-yl)-4-((3- fluoropyridin-2- yl)thio)pyrazolo[1,5- a]pyridine-3-carbonitrile | Example 39 | 492.2 | 5 |
| 235 | 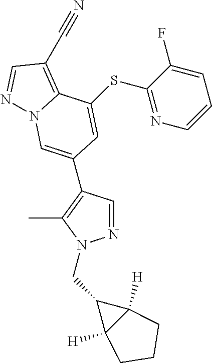 [SMILES: Cc1c(-c2cc(Sc3ncccc3F)c3c(C#N)cnn3c2)cnn1C[C@H]1[C@@H]2CCC[C@@H]21] | 6-(1-(((1R,5S,6r)- bicyclo[3.1.0]hexan-6- yl)methyl)-5-methyl-1H- pyrazol-4-yl)-4-((3- fluoropyridin-2- yl)thio)pyrazolo[1,5- a]pyridine-3-carbonitrile | Example 53 | 446.1 | 4 |
| 236 | 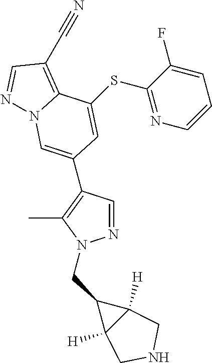 [SMILES: Cc1c(-c2cc(Sc3ncccc3F)c3c(C#N)cnn3c2)cnn1C[C@@H]1[C@@H]2CNC[C@@H]21] | 6-(1-(((1R,5S,6s)-3- azabicyclo[3.1.0]hexan-6- yl)methyl)-5-methyl-1H- pyrazol-4-yl)-4-((3- fluoropyridin-2- yl)thio)pyrazolo[1,5- a]pyridine-3-carbonitrile | Example 53 | 446.3 | 1 |
| 237 | 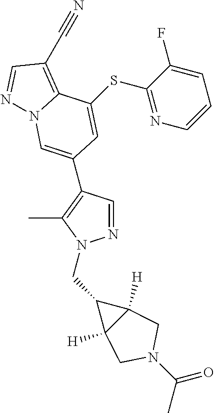 [SMILES: CC(=O)N1C[C@@H]2[C@H](C1)[C@H]2Cn1ncc(-c2cc(Sc3ncccc3F)c3c(C#N)cnn3c2)c1C] | 6-(1-(((1R,5S,6r)-3- acetyl-3- azabicyclo[3.1.0]hexan-6- yl)methyl)-5-methyl-1H- pyrazol-4-yl)-4-((3- fluoropyridin-2- yl)thio)pyrazolo[1,5- a]pyridine-3-carbonitrile | Example 39 | 488.2 | 3 |
| 238 | 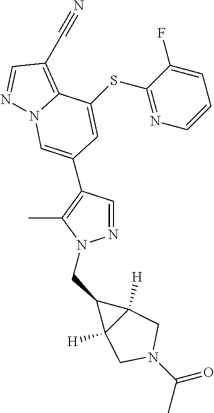 [SMILES: CC(=O)N1C[C@@H]2[C@H](C1)[C@@H]2Cn1ncc(-c2cc(Sc3ncccc3F)c3c(C#N)cnn3c2)c1C] | 6-(1-(((1R,5S,6s)-3- acetyl-3- azabicyclo[3.1.0]hexan-6- yl)methyl)-5-methyl-1H- pyrazol-4-yl)-4-((3- fluoropyridin-2- yl)thio)pyrazolo[1,5- a]pyridine-3-carbonitrile | Exmaple 39 | 488.4 | 3 |
| 239 | 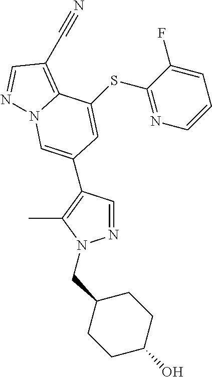 [SMILES: Cc1c(-c2cc(Sc3ncccc3F)c3c(C#N)cnn3c2)cnn1C[C@H]1CC[C@H](O)CC1] | 4-((3-fluoropyridin-2- yl)thio)-6-(1-(((1r,4r)-4- hydroxycyclohexyl)methyl)- 5-methyl-1H-pyrazol-4- yl)pyrazolo[1,5- a]pyridine-3-carbonitrile | Example 53 | 463.1 | 3 |
| 240 | 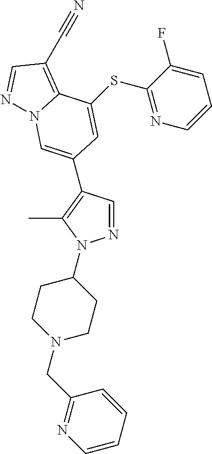 [SMILES: Cc1c(-c2cc(Sc3ncccc3F)c3c(C#N)cnn3c2)cnn1C1CCN(Cc2ccccn2)CC1] | 4-((3-fluoropyridin-2- yl)thio)-6-(5-methyl-1-(1- (pyridin-2- ylmethyl)piperidin-4-yl)- 1H-pyrazol-4- yl)pyrazolo[1,5- a]pyridine-3-carbonitrile | Example 35 | 525.2 | 2 |
| 241 | 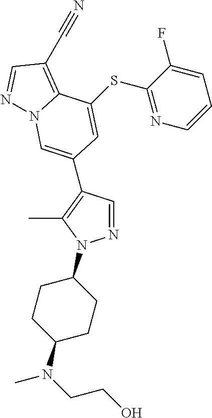 [SMILES: Cc1c(-c2cc(Sc3ncccc3F)c3c(C#N)cnn3c2)cnn1[C@H]1CC[C@@H](N(C)CCO)CC1] | 4-((3-fluoropyridin-2- yl)thio)-6-(1-((1s,4s)-4- ((2- hydroxyethyl)(methyl)ami- no)cyclohexyl)-5-methyl- 1H-pyrazol-4- yl)pyrazolo[1,5- a]pyridine-3-carbonitrile | Example 35 | 506.1 | 1 |
| 242 | 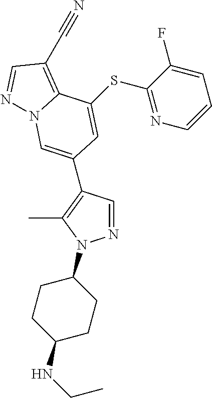 [SMILES: CCN[C@H]1CC[C@@H](n2ncc(-c3cc(Sc4ncccc4F)c4c(C#N)cnn4c3)c2C)CC1] | 6-(1-((1s,4s)-4- (ethylamino)cyclohexyl)- 5-methyl-1H-pyrazol-4- yl)-4-((3-fluoropyridin-2- yl)thio)pyrazolo[1,5- a]pyridine-3-carbonitrile | Example 32 | 476.2 | 1 |
| 243 | 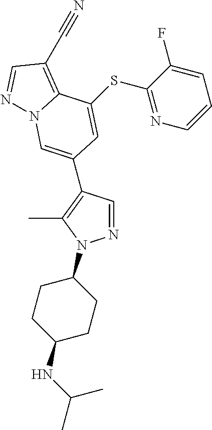 [SMILES: Cc1c(-c2cc(Sc3ncccc3F)c3c(C#N)cnn3c2)cnn1[C@H]1CC[C@@H](NC(C)C)CC1] | 4-((3-fluoropyridin-2- yl)thio)-6-(1-((1s,4s)-4- (isopropylamino)cyclohex- yl)-5-methyl-1H-pyrazol- 4-yl)pyrazolo[1,5- a]pyridine-3-carbonitrile | Example 32 | 490.3 | 1 |
| 244 | 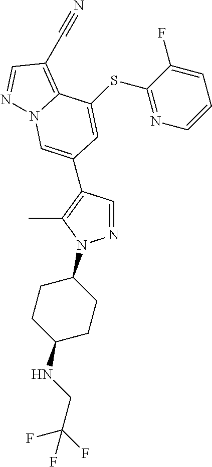 [SMILES: Cc1c(-c2cc(Sc3ncccc3F)c3c(C#N)cnn3c2)cnn1[C@H]1CC[C@@H](NCC(F)(F)F)CC1] | 4-((3-fluoropyridin-2- yl)thio)-6-(5-methyl-1- ((1s,4s)-4-((2,2,2- trifluoroethyl)amino)cyclo- hexyl)-1H-pyrazol-4- yl)pyrazolo[1,5- a]pyridine-3-carbonitrile | Example 35 | 580.3 | 2.5 |
| 245 | 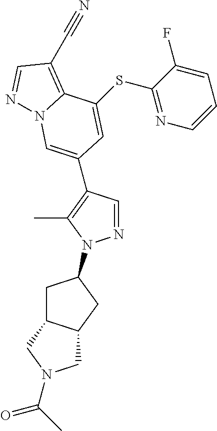 [SMILES: CC(=O)N1C[C@H]2C[C@H](n3ncc(-c4cc(Sc5ncccc5F)c5c(C#N)cnn5c4)c3C)C[C@H]2C1] | 6-(1-((3aR,5s,6aS)-2- acetyloctahydrocylcopenta [c]pyrrol-5-yl)-5-methyl- 1H-pyrazol-4-yl)-4-((3- fluoropyridin-2- yl)thio)pyrazolo[1,5- a]pyridine-3-carbonitrile | Example 39 | 502.1 | 2.5 |
| 246 | 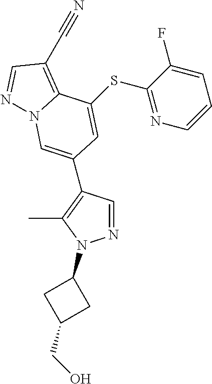 [SMILES: Cc1c(-c2cc(Sc3ncccc3F)c3c(C#N)cnn3c2)cnn1[C@H]1C[C@H](CO)C1] | 4-((3-fluoropyridin-2- yl)thio)-6-(1-((1r,3r)-3- (hydroxymethyl)cyclobutyl)- 5-methyl-1H-pyrazol-4- yl)pyrazolo[1,5- a]pyridine-3-carbonitrile | Example 53 | 435.3 | 4 |
| 247 | 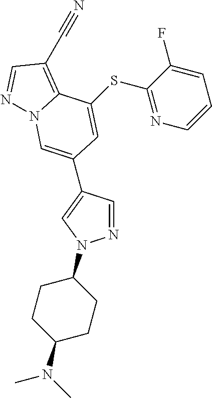 [SMILES: CN(C)[C@H]1CC[C@@H](n2cc(-c3cc(Sc4ncccc4F)c4c(C#N)cnn4c3)cn2)CC1] | 6-(1-((1s,4s)-4- (dimethylamino)cyclohex- yl)-1H-pyrazol-4-yl)-4- ((3-fluoropyridin-2- yl)thio)pyrazolo[1,5- a]pyridine-3-carbonitrile | Example 32 | 462.3 | 1 |
| 248 | 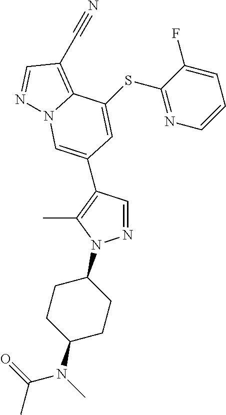 [SMILES: CC(=O)N(C)[C@H]1CC[C@@H](n2ncc(-c3cc(Sc4ncccc4F)c4c(C#N)cnn4c3)c2C)CC1] | N-((1s,4s)-4-(4-(3-cyano- 4-((3-fluoropyridin-2- yl)thio)pyrazolo[1,5- a]pyridin-6-yl)-5-methyl- 1H-pyrazol-1- yl)cyclohexyl)-N- methylacetamide | Example 39 | 504.3 | 2 |
| 249 | 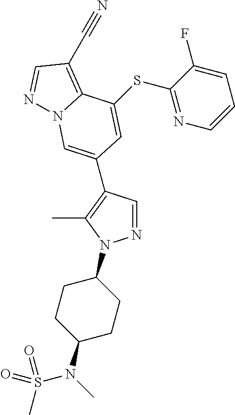 [SMILES: Cc1c(-c2cc(Sc3ncccc3F)c3c(C#N)cnn3c2)cnn1[C@H]1CC[C@@H](N(C)S(C)(=O)=O)CC1] | N-((1s,4s)-4-(4-(3-cyano- 4-((3-fluoropyridin-2- yl)thio)pyrazolo[1,5- a]pyridin-6-yl)-5-methyl- 1H-pyrazol-1- yl)cyclohexyl)-N- methylmethanesulfonamide | Exmaple 50 | 540.2 | 3 |
| 250 | 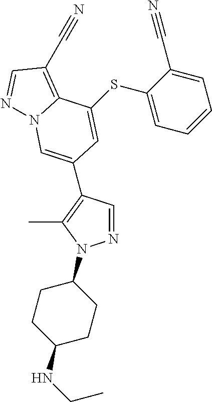 [SMILES: CCN[C@H]1CC[C@@H](n2ncc(-c3cc(Sc4ccccc4C#N)c4c(C#N)cnn4c3)c2C)CC1] | 4-((2-cyanophenyl)thio)- 6-(1-((1s,4s)-4- (ethylamino)cylcohexyl)- 5-methyl-1H-pyrazol-4- yl)pyrazolo[1,5- a]pyridine-3-carbonitrile | Example 32 | 482.2 | 0.4 |
| 251 | 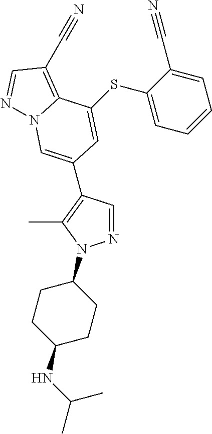 [SMILES: Cc1c(-c2cc(Sc3ccccc3C#N)c3c(C#N)cnn3c2)cnn1[C@H]1CC[C@@H](NC(C)C)CC1] | 6-(1-((1s,4s)-4- (ethylamino)cyclohexyl)- 5-methyl-1H-pyrazol-4- yl)-4-((3-fluoropyridin-2- yl)thio)pyrazolo[1,5- a]pyridine-3-carbonitrile | Example 53 | 496.2 | 0.5 |
| 252 | 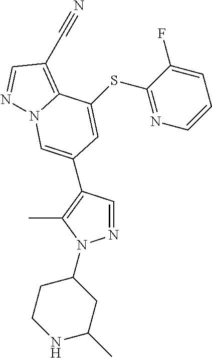 [SMILES: Cc1c(-c2cc(Sc3ncccc3F)c3c(C#N)cnn3c2)cnn1C1CCNC(C)C1] | 4-((3-fluoropyridin-2- yl)thio)-6-(5-methyl-1-(2- methylpiperidin-4-yl)-1H- pyrazol-4-yl)pyrazolo[1,5- a]pyridine-3-carbonitrile | Example 53 | 448.1 | 1 |
| 253 | 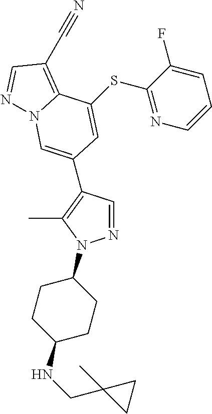 [SMILES: Cc1c(-c2cc(Sc3ncccc3F)c3c(C#N)cnn3c2)cnn1[C@H]1CC[C@@H](NCC2(C)CC2)CC1] | 4-((3-fluoropyridin-2- yl)thio)-6-(5-methyl-1- ((1s,4s)-4-(((1- methylcyclopropyl)methyl) amino)cyclohexyl)-1H- pyrazol-4-yl)pyrazolo[1,5- a]pyridine-3-carbonitrile | Example 32 | 516.3 | 0.6 |
| 254 | 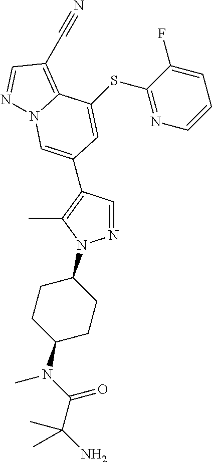 [SMILES: Cc1c(-c2cc(Sc3ncccc3F)c3c(C#N)cnn3c2)cnn1[C@H]1CC[C@@H](N(C)C(=O)C(C)(C)N)CC1] | 2-amino-N-((1s,4s)-4-(4- (3-cyano-4-((3- fluoropyridin-2- yl)thio)pyrazolo[1,5- a]pyridin-6-yl)-5-methyl- 1H-pyrazol-1- y)cyclohexyl)-N,2- dimethylpropanamide | Example 36 | 547.4 | 1 |
| 255 | 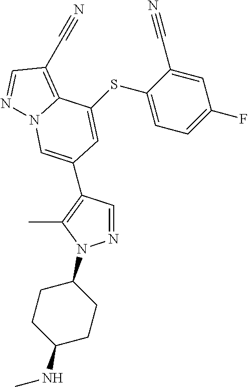 [SMILES: CN[C@H]1CC[C@@H](n2ncc(-c3cc(Sc4ccc(F)cc4C#N)c4c(C#N)cnn4c3)c2C)CC1] | 4-((2-cyano-4- fluorophenyl)thio)-6-(5- methyl-1-((1s,4s)-4- (methylamino)cyclohexyl)- 1H-pyrazol-4- yl)pyrazolo[1,5- a]pyridine-3-carbonitrile | Example 10 | 486.3 | 0.3 |
| 256 | 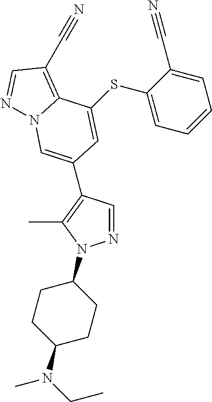 [SMILES: CCN(C)[C@H]1CC[C@@H](n2ncc(-c3cc(Sc4ccccc4C#N)c4c(C#N)cnn4c3)c2C)CC1] | 4-((2-cyanophenyl)thio)- 6-(1-((1s,4s)-4- (ethyl(methyl)amino)cyclo- hexyl)-5-methyl-1H- pyrazol-4-yl)pyrazolo[1,5- a]pyridine-3-carbonitrile | Example 32 | 496.4 | 1 |
| 257 | 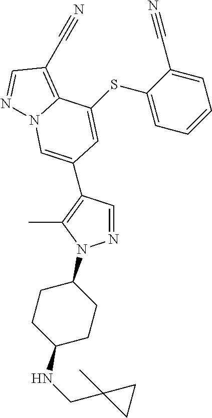 [SMILES: Cc1c(-c2cc(Sc3ccccc3C#N)c3c(C#N)cnn3c2)cnn1[C@H]1CC[C@@H](NCC2(C)CC2)CC1] | 4-((2-cyanophenyl)thio)- 6-(5-methyl-1-((1s,4s)-4- (((1- methylcyclopropyl)methyl) amino)cyclohexyl)-1H- pyrazol-4-yl)pyrazolo[1,5- a]pyridine-3-carbonitrile | Example 32 | 522.4 | 0.5 |
| 258 | 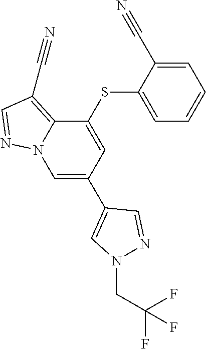 [SMILES: N#Cc1ccccc1Sc1cc(-c2cnn(CC(F)(F)F)c2)cn2ncc(C#N)c12] | 4-((2-cyanophenyl)thio)- 6-(1-(2,2,2-trifluoroethyl)- 1H-pyrazol-4- yl)pyrazolo[1,5- a]pyridine-3-carbonitrile | Example 53 | 425.1 | 2 |
| 259 | 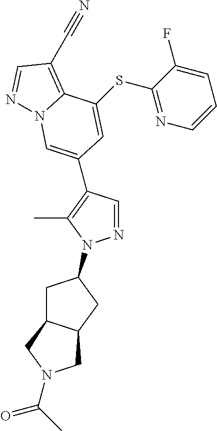 [SMILES: CC(=O)N1C[C@H]2C[C@@H](n3ncc(-c4cc(Sc5ncccc5F)c5c(C#N)cnn5c4)c3C)C[C@H]2C1] | 6-(1-((3aR,5r,6aS)-2- acetyloctahydrocyclopenta [c]pyrrol-5-yl)-5-methyl- 1H-pyrazol-4-yl)-4-((3- fluoropyridin-2- yl)thio)pyrazolo[1,5- a]pyridine-3-carbonitrile | Example 39 | 502.2 | 1.5 |
| 260 | 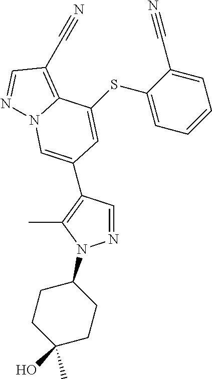 [SMILES: Cc1c(-c2cc(Sc3ccccc3C#N)c3c(C#N)cnn3c2)cnn1[C@H]1CC[C@@](C)(O)CC1] | 4-((2-cyanophenyl)thio)- 6-(1-((1s,4s)-4-hydroxy-4- methylcyclohexyl)-5- methyl-1H-pyrazol-4- yl)pyrazolo[1,5- a]pyridine-3-carbonitrile | Example 54 | 469.3 | 1 |
| 261 | 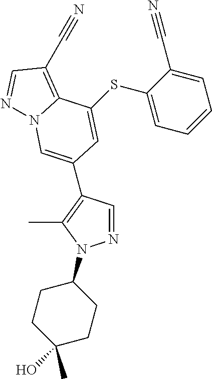 [SMILES: Cc1c(-c2cc(Sc3ccccc3C#N)c3c(C#N)cnn3c2)cnn1[C@H]1CC[C@](C)(O)CC1] | 4-((2-cyanophenyl)thio)- 6-(1-((1r,4r)-4-hydroxy-4- methylcyclohexyl)-5- methyl-1H-pyrazol-4- yl)pyrazolo[1,5- a]pyridine-3-carbonitrile | Example 54 | 469.3 | 0.5 |
| 262 | 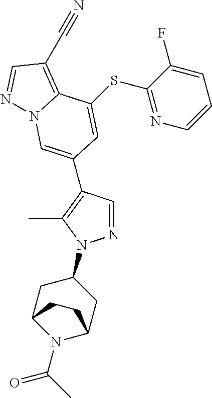 [SMILES: CC(=O)N1[C@@H]2CC[C@H]1C[C@@H](n1ncc(-c3cc(Sc4ncccc4F)c4c(C#N)cnn4c3)c1C)C2] | 6-(1-((1R,3r,5S)-8-acetyl- 8-azabicyclo[3.2.1]octan- 3-yl)-5-methyl-1H- pyrazol-4-yl)-4-((3- fluoropyridin-2- yl)thio)pyrazolo[1,5- a]pyridine-3-carbonitrile | Example 32 | 502.2 | 3 |
| 263 | 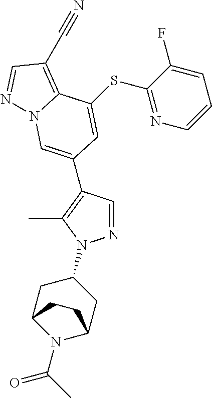 [SMILES: CC(=O)N1[C@@H]2CC[C@H]1C[C@H](n1ncc(-c3cc(Sc4ncccc4F)c4c(C#N)cnn4c3)c1C)C2] | 6-(1-((1R,3s,5S)-8-acetyl- 8-azabicyclo[3.2.1]octan- 3-yl)-5-methyl-1H- pyrazol-4-yl)-4-((3- fluoropyridin-2- yl)thio)pyrazolo[1,5- a]pyridine-3-carbonitrile | Example 32 | 502.2 | 3 |
| 264 | 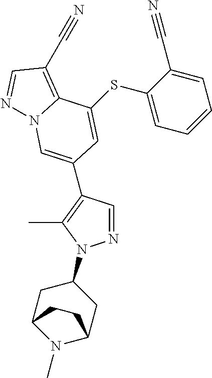 [SMILES: Cc1c(-c2cc(Sc3ccccc3C#N)c3c(C#N)cnn3c2)cnn1[C@H]1C[C@H]2CC[C@@H](C1)N2C] | 4-((2-cyanophenyl)thio)- 6-(5-methyl-1- ((1R,3r,5S)-8-methyl-8- azabicyclo[3.2.1]octan-3- yl)-1H-pyrazol-4- yl)pyrazolo[1,5- a]pyridine-3-carbonitrile | Example 53 | 480.4 | 1 |
| 265 | 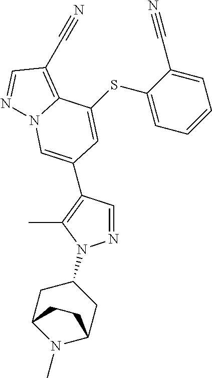 [SMILES: Cc1c(-c2cc(Sc3ccccc3C#N)c3c(C#N)cnn3c2)cnn1[C@@H]1C[C@H]2CC[C@@H](C1)N2C] | 4-((2-cyanophenyl)thio)- 6-(5-methyl-1- ((1R,3s,5S)-8-methyl-8- azabicyclo[3.2.1]octan-3- yl)-1H-pyrazol-4- yl)pyrazolo[1,5- a]pyridine-3-carbonitrile | Example 53 | 480.3 | 0.4 |
| 266 | 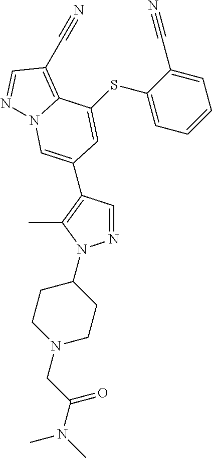 [SMILES: Cc1c(-c2cc(Sc3ccccc3C#N)c3c(C#N)cnn3c2)cnn1C1CCN(CC(=O)N(C)C)CC1] | 2-(4-(4-(3-cyano-4-((2- cyanophenyl)thio)pyrazolo [1,5-a]pyridin-6-yl)-5- methyl-1H-pyrazol-1- yl)piperidin-1-yl)-N,N- dimethylacetamide | Example 35 | 525.4 | 0.5 |
| 267 | 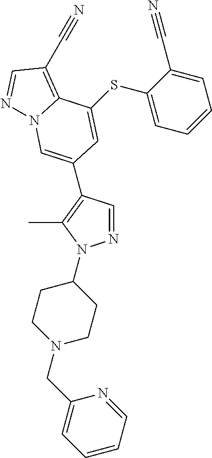 [SMILES: Cc1c(-c2cc(Sc3ccccc3C#N)c3c(C#N)cnn3c2)cnn1C1CCN(Cc2ccccn2)CC1] | 4-((2-cyanophenyl)thio)- 6-(5-methyl-1-(1-(pyridin- 2-ylmethyl)piperidin-4- yl)-1H-pyrazol-4- yl)pyrazolo[1,5- a]pyridine-3-carbonitrile | Example 35 | 531.2 | 0.6 |
| 268 | 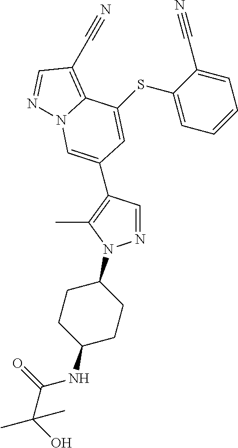 [SMILES: Cc1c(-c2cc(Sc3ccccc3C#N)c3c(C#N)cnn3c2)cnn1[C@H]1CC[C@@H](NC(=O)C(C)(C)O)CC1] | N-((1s,4s)-4-(4-(3-cyano- 4-((2- cyanophenyl)thio)pyrazolo [1,5-a]pyridin-6-yl)-5- methyl-1H-pyrazol-1- yl)cyclohexyl)-2-hydroxy- 2-methylpropanamide | Example 36 | 540.2 | 0.4 |
| 269 | 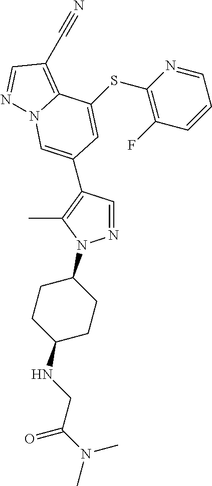 [SMILES: Cc1c(-c2cc(Sc3ncccc3F)c3c(C#N)cnn3c2)cnn1[C@H]1CC[C@@H](NCC(=O)N(C)C)CC1] | 2-(((1s,4s)-4-(4-(3-cyano- 4-((3-fluoropyridin-2- yl)thio)pyrazolo[1,5- a]pyridin-6-yl)-5-methyl- 1H-pyrazol-1- yl)cyclohexyl)amino)- N,N-dimethylacetamide | Example 35 | 533.2 | 1 |
| 270 | 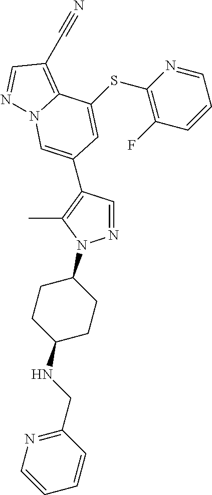 [SMILES: Cc1c(-c2cc(Sc3ncccc3F)c3c(C#N)cnn3c2)cnn1[C@H]1CC[C@@H](NCc2ccccn2)CC1] | 4-((3-fluoropyridin-2- yl)thio)-6-(5-methyl-1- ((1s,4s)-4-((pyridin-2- ylmethyl)amino)cyclohex- yl)-1H-pyrazol-4- yl)pyrazolo[1,5- a]pyridine-3-carbonitrile | Example 35 | 539.4 | 0.7 |
| 271 | 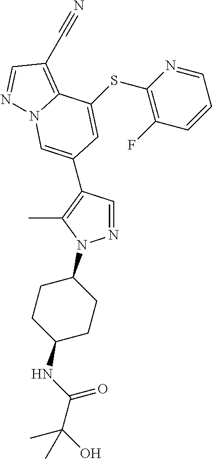 [SMILES: Cc1c(-c2cc(Sc3ncccc3F)c3c(C#N)cnn3c2)cnn1[C@H]1CC[C@@H](NC(=O)C(C)(C)O)CC1] | N-((1s,4s)-4-(4-(3-cyano- 4-((3-fluoropyridin-2- yl)thio)pyrazolo[1,5- a]pyridin-6-yl)-5-methyl- 1H-pyrazol-1- yl)cyclohexyl)-2-hydroxy- 2-methylpropanamide | Example 36 | 534.4 | 1 |
| 272 | 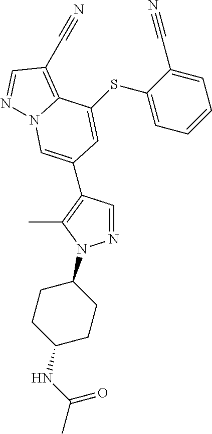 [SMILES: CC(=O)N[C@H]1CC[C@H](n2ncc(-c3cc(Sc4ccccc4C#N)c4c(C#N)cnn4c3)c2C)CC1] | N-((1r,4r)-4-(4-(3-cyano- 4-((2- cyanophenyl)thio)pyrazolo [1,5-a]pyridin-6-yl)-5- methyl-1H-pyrazol-1- yl)cyclohexyl)acetamide | Example 39 | 496.3 | 1 |
| 273 | 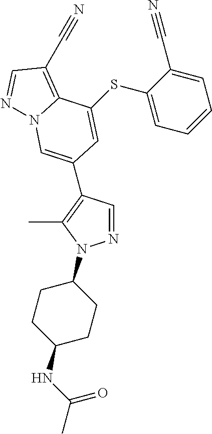 [SMILES: CC(=O)N[C@H]1CC[C@@H](n2ncc(-c3cc(Sc4ccccc4C#N)c4c(C#N)cnn4c3)c2C)CC1] | N-((1s,4s)-4-(4-(3-cyano- 4-((2- cyanophenyl)thio)pyrazolo [1,5-a]pyridin-6-yl)-5- methyl-1H-pyrazol-1- yl)cyclohexyl)acetamide | Example 39 | 496.2 | 0.5 |
| 274 | 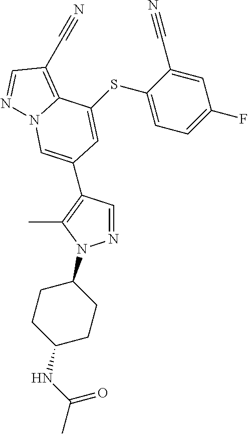 [SMILES: CC(=O)N[C@H]1CC[C@H](n2ncc(-c3cc(Sc4ccc(F)cc4C#N)c4c(C#N)cnn4c3)c2C)CC1] | N-((1r,4r)-4-(4-(3-cyano- 4-((2-cyano-4- fluorophenyl)thio)pyrazolo [1,5-a]pyridin-6-yl)-5- methyl-1H-pyrazol-1- yl)cyclohexyl)acetamide | Example 39 | 541.3 | 1 |
| 275 | 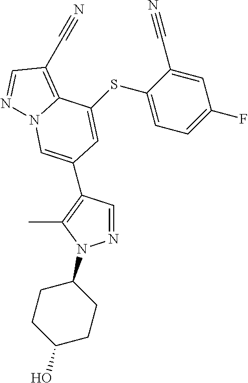 [SMILES: Cc1c(-c2cc(Sc3ccc(F)cc3C#N)c3c(C#N)cnn3c2)cnn1[C@H]1CC[C@H](O)CC1] | 4-((2-cyano-4- fluorophenyl)thio)-6-(1- ((1r,4r)-4- hydroxycyclohexyl)-5- methyl-1H-pyrazol-4- yl)pyrazolo[1,5- a]pyridine-3-carbonitrile | Example 58 | 473.3 | 1 |
| 276 | 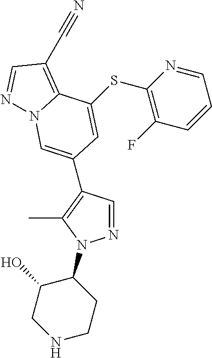 [SMILES: Cc1c(-c2cc(Sc3ncccc3F)c3c(C#N)cnn3c2)cnn1[C@H]1CCNC[C@@H]1O] | 4-((3-fluoropyridin-2- yl)thio)-6-(1-((3S,4S)-3- hydroxypiperidin-4-yl)-5- methyl-1H-pyrazol-4- yl)pyrazolo[1,5- a]pyridine-3-carbonitrile | Example 55 | 449.9 | 3 |
| 277 | 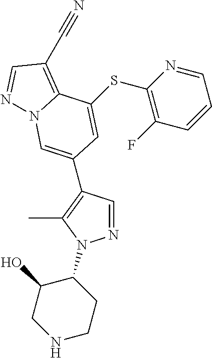 [SMILES: Cc1c(-c2cc(Sc3ncccc3F)c3c(C#N)cnn3c2)cnn1[C@@H]1CCNC[C@H]1O] | 4-((3-fluoropyridin-2- yl)thio)-6-(1-((3R,4R)-3- hydroxypiperidin-4-yl)-5- methyl-1H-pyrazol-4- yl)pyrazolo[1,5- a]pyridine-3-carbonitrile | Example 55 | 450.0 | 15 |
| 278 | 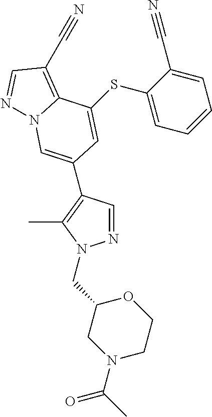 [SMILES: CC(=O)N1CCO[C@@H](Cn2ncc(-c3cc(Sc4ccccc4C#N)c4c(C#N)cnn4c3)c2C)C1] | (R)-6-(1-((4- acetylmorpholin-2- yl)methyl)-5-methyl-1H- pyrazol-4-yl)-4-((2- cyanophenyl)thio)pyrazolo [1,5-a]pyridine-3- carbonitrile | Example 39 | 498.3 | 4 |
| 279 | 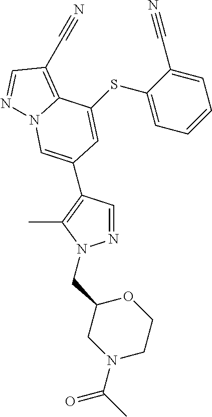 [SMILES: CC(=O)N1CCO[C@H](Cn2ncc(-c3cc(Sc4ccccc4C#N)c4c(C#N)cnn4c3)c2C)C1] | (S)-6-(1-((4- acetylmorpholin-2- yl)methyl)-5-methyl-1H- pyrazol-4-yl)-4-((2- cyanophenyl)thio)pyrazolo [1,5-a]pyridine-3- carbonitrile | Example 39 | 498.2 | 3 |
| 280 | 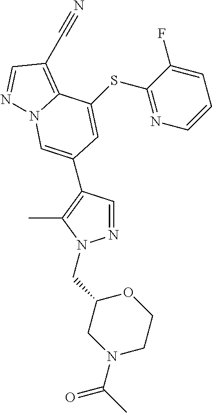 [SMILES: CC(=O)N1CCO[C@@H](Cn2ncc(-c3cc(Sc4ncccc4F)c4c(C#N)cnn4c3)c2C)C1] | (R)-6-(1-((4- acetylmorpholin-2- yl)methyl)-5-methyl-1H- pyrazol-4-yl)-4-((3- fluoropyridin-2- yl)thio)pyrazolo[1,5- a]pyridine-3-carbonitrile | Example 39 | 492.3 | 12 |
| 281 | 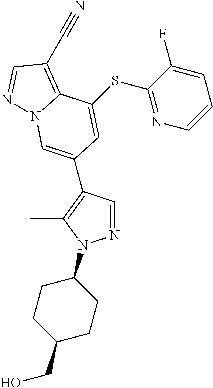 [SMILES: Cc1c(-c2cc(Sc3ncccc3F)c3c(C#N)cnn3c2)cnn1[C@H]1CC[C@@H](CO)CC1] | 4-((3-fluoropyridin-2- yl)thio)-6-(1-((1s,4s)-4- (hydroxymethyl)cyclohex- yl)-5-methyl-1H-pyrazol- 4-yl)pyrazolo[1,5- a]pyridine-3-carbonitril | Example 53 | 463.2 | 2 |
| 282 | 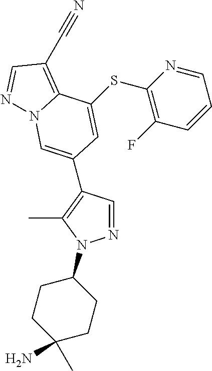 [SMILES: Cc1c(-c2cc(Sc3ncccc3F)c3c(C#N)cnn3c2)cnn1[C@H]1CC[C@@](C)(N)CC1] | 6-(1-((1s,4s)-4-amino-4- methylcyclohexyl)-5- methyl-1H-pyrazol-4-yl)- 4-((3-fluoropyridin-2- yl)thio)pyrazolo[1,5- a]pyridine-3-carbonitrile | Example 53 | 462.3 | 1 |
| 283 | 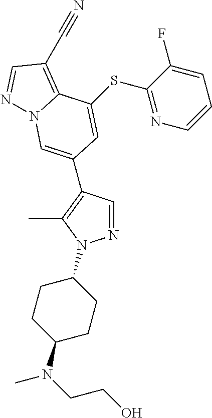 [SMILES: Cc1c(-c2cc(Sc3ncccc3F)c3c(C#N)cnn3c2)cnn1[C@H]1CC[C@H](N(C)CCO)CC1] | 4-((3-fluoropyridin-2- yl)thio)-6-(1-((1r,4r)-4- ((2- hydroxyethyl)(methyl)ami- no)cyclohexyl)-5-methyl- 1H-pyrazol-4- yl)pyrazolo[1,5- a]pyridine-3-carbonnitrile | Example 35 | 509.2 | 1 |
| 284 | 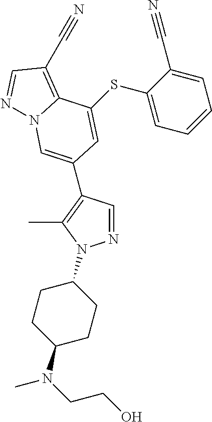 [SMILES: Cc1c(-c2cc(Sc3ccccc3C#N)c3c(C#N)cnn3c2)cnn1[C@H]1CC[C@H](N(C)CCO)CC1] | 4-((2-cyanophenyl)thio)- 6-(1-((1r,4SR)-4-((2- hydroxyethyl)(methyl)ami- no)cyclohexyl)-5-methyl- 1H-pyrazol-4- yl)pyrazolo[1,5- a]pyridine-3-carbonitrile | Example 35 | 512.4 | 0.4 |
| 285 | 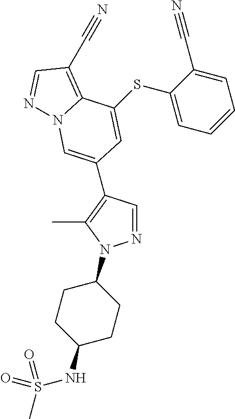 [SMILES: Cc1c(-c2cc(Sc3ccccc3C#N)c3c(C#N)cnn3c2)cnn1[C@H]1CC[C@@H](NS(C)(=O)=O)CC1] | N-((1s,4s)-4-(4-(3-cyano- 4-((2- cyanophenyl)thio)pyrazolo [1,5-a]pyridin-6-yl)-5- methyl-1H-pyrazol-1- y)cyclohexyl)methane- sulfonamide | Example 50 | 532.4 | 1 |
| 286 | 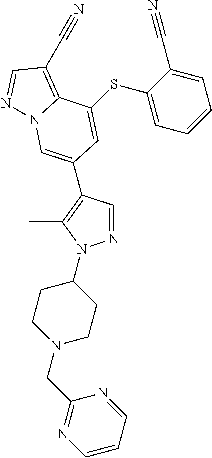 [SMILES: Cc1c(-c2cc(Sc3ccccc3C#N)c3c(C#N)cnn3c2)cnn1C1CCN(Cc2ncccn2)CC1] | 4-((2-cyanophenyl)thio)- 6-(5-methyl-1-(1- (pyrimidin-2- ylmethyl)piperidin-4-yl)- 1H-pyrazol-4- yl)pyrazolo[1,5- a]pyridine-3-carbonitrile | Example 35 | 532.4 | 1 |
| 287 | 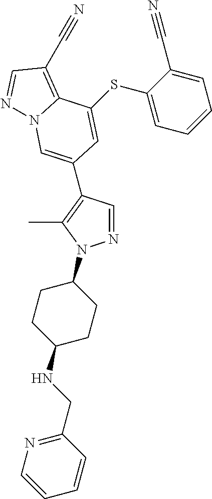 [SMILES: Cc1c(-c2cc(Sc3ccccc3C#N)c3c(C#N)cnn3c2)cnn1[C@H]1CC[C@@H](NCc2ccccn2)CC1] | 4-((2-cyanophenyl)thio)- 6-(5-methyl-1-((1s,4s)-4- ((pyridin-2- ylmethyl)amino)cyclohex- yl)-1H-pyrazol-4- yl)pyrazolo[1,5- a]pyridine-3-carbonitrile | Example 35 | 545.4 | 0.4 |
| 288 | 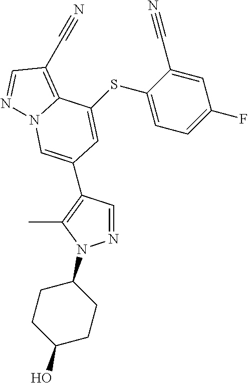 [SMILES: Cc1c(-c2cc(Sc3ccc(F)cc3C#N)c3c(C#N)cnn3c2)cnn1[C@H]1CC[C@@H](O)CC1] | 4-((2-cyano-4- fluorophenyl)thio)-6-(1- ((1s,4s)-4- hydroxycyclohexyl)-5- methyl-1H-pyrazol-4- yl)pyrazolo[1,5- a]pyridine-3-carbonitrile | Example 53 | 473.1 | 1 |
| 289 | 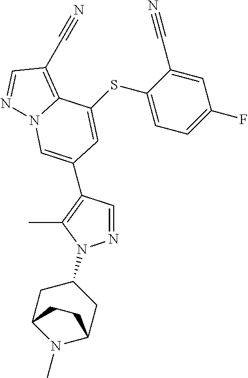 [SMILES: Cc1c(-c2cc(Sc3ccc(F)cc3C#N)c3c(C#N)cnn3c2)cnn1[C@@H]1C[C@H]2CC[C@@H](C1)N2C] | 4-((2-cyano-4- fluorophenyl)thio)-6-(5- methyl-1-((1R,3s,5S)-8- methyl-8- azabicyclo[3.2.1]octan-3- yl)-1H-pyrazol-4- yl)pyrazolo[1,5- a]pyridine-3-carbonitrile | Example 53 | 498.3 | 0.5 |
| 290 | 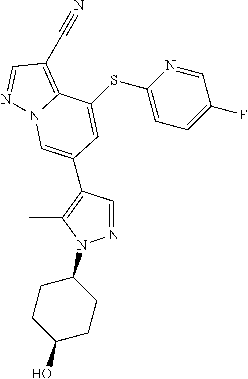 [SMILES: Cc1c(-c2cc(Sc3ccc(F)cn3)c3c(C#N)cnn3c2)cnn1[C@H]1CC[C@@H](O)CC1] | 4-((5-fluoropyridin-2- yl)thio)-6-(1-((1s,4s)-4- hydroxycyclohexyl)-5- methyl-1H-pyrazol-4- yl)pyrazolo[1,5- a]pyridine-3-carbonitrile | Example 53 | 449.1 | 5 |
| 291 | 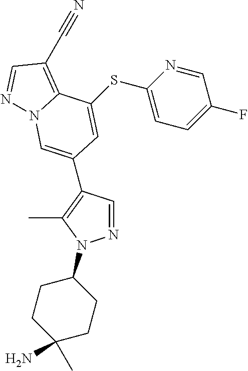 [SMILES: Cc1c(-c2cc(Sc3ccc(F)cn3)c3c(C#N)cnn3c2)cnn1[C@H]1CC[C@@](C)(N)CC1] | 6-(1-((1s,4s)-4-amino-4- methylcyclohexyl)-5- methyl-1H-pyrazol-4-yl)- 4-((5-fluoropyridin-2- yl)thio)pyrazolo[1,5- a]pyridine-3-carbonitrile | Example 53 | 462.2 | 3 |
| 292 | 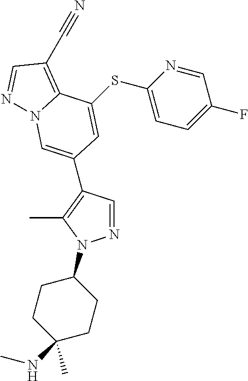 [SMILES: CN[C@]1(C)CC[C@H](n2ncc(-c3cc(Sc4ccc(F)cn4)c4c(C#N)cnn4c3)c2C)CC1] | 4-((5-fluoropyridin-2- yl)thio)-6-(5-methyl-1- ((1s,4s)-4-methyl-4- (methylamino)cyclohexyl)- 1H-pyrazol-4- yl)pyrazolo[1,5- a]pyridine-3-carbonitrile | Example 53 | 476.3 | 3 |
| 293 | 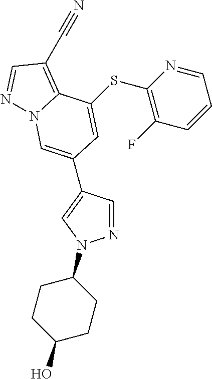 [SMILES: N#Cc1cnn2cc(-c3cnn([C@H]4CC[C@@H](O)CC4)c3)cc(Sc3ncccc3F)c12] | 4-((3-fluoropyridin-2- yl)thio)-6-(1-((1s,4s)-4- hydroxycyclohexyl)-1H- pyrazol-4-yl)pyrazolo[1,5- a]pyridine-3-carbonitrile | Example 53 | 435.2 | 1 |
| 294 | 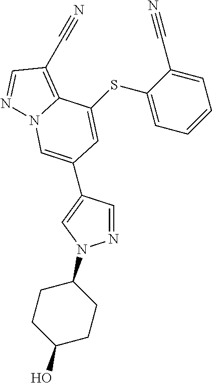 [SMILES: N#Cc1ccccc1Sc1cc(-c2cnn([C@H]3CC[C@@H](O)CC3)c2)cn2ncc(C#N)c12] | 4-((2-cyanophenyl)thio)- 6-(1-((1s,4s)-4- hydroxycyclohexyl)-1H- pyrazol-4-yl)pyrazolo[1,5- a]pyridine-3-carbonitrile | Example 53 | 441.1 | 0.5 |
| 295 | 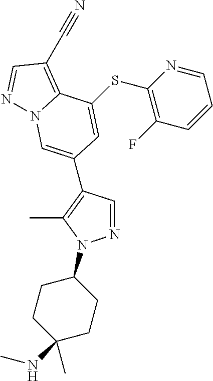 [SMILES: CN[C@]1(C)CC[C@H](n2ncc(-c3cc(Sc4ncccc4F)c4c(C#N)cnn4c3)c2C)CC1] | 4-((3-fluoropyridin-2- yl)thio)-6-(5-methyl-1- ((1s,4s)-4-methyl-4- (methylamino)cylcohexyl)- 1H-pyrazol-4- yl)pyrazolo[1,5- a]pyridine-3-carbonitrile | Example 53 | 476.2 | 1 |
| 296 | 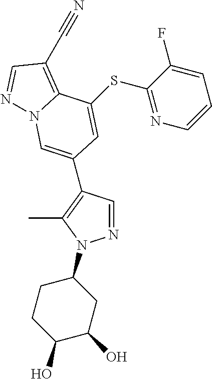 [SMILES: Cc1c(-c2cc(Sc3ncccc3F)c3c(C#N)cnn3c2)cnn1[C@@H]1CC[C@H](O)[C@H](O)C1] | 6-(1-((1R,3R,4S)-3,4- dihydroxycyclohexyl)-5- methyl-1H-pyrazol-4-yl)- 4-((3-fluoropyridin-2- yl)thio)pyrazolo[1,5- a]pyridine-3-carbonitrile | Example 56 | 465.1 | 2 |
| 297 | 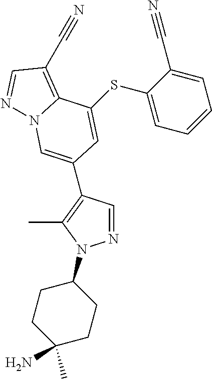 [SMILES: Cc1c(-c2cc(Sc3ccccc3C#N)c3c(C#N)cnn3c2)cnn1[C@H]1CC[C@@](C)(N)CC1] | 4-((3-fluoropyridin-2- yl)thio)-6-(1-((1r,3r)-3- (hydroxymethyl)cyclobutyl)- 5-methyl-1H-pyrazol-4- yl)pyrazolo[1,5- a]pyridine-3-carbonitrile | Example 53 | 468.4 | 0.3 |
| 298 | 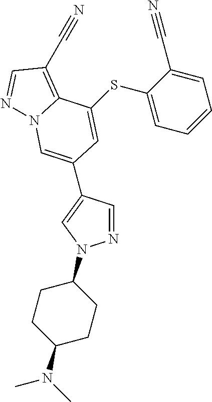 [SMILES: CN(C)[C@H]1CC[C@@H](n2cc(-c3cc(Sc4ccccc4C#N)c4c(C#N)cnn4c3)cn2)CC1] | 4-((2-cyanophenyl)thio)- 6-(1-((1s,4s)-4- (dimethylamino)cyclohex- yl)-1H-pyrazol-4- yl)pyrazolo[1,5- a]pyridine-3-carbonitrile | Example 32 | 468.4 | 0.5 |
| 299 | 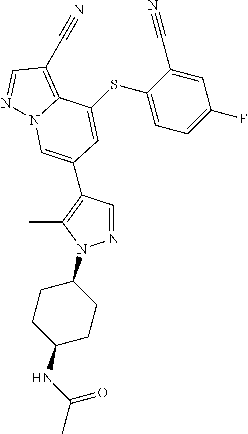 [SMILES: CC(=O)N[C@H]1CC[C@@H](n2ncc(-c3cc(Sc4ccc(F)cc4C#N)c4c(C#N)cnn4c3)c2C)CC1] | N-((1s,4S)-4-(4-(3-cyano- 4-((2-cyano-4- fluorophenyl)thio)pyrazolo [1,5-a]pyridin-6-yl)-5- methyl-1H-pyrazol-1- yl)cyclohexyl)acetamide | Example 39 | 514.3 | 1 |
| 300 | 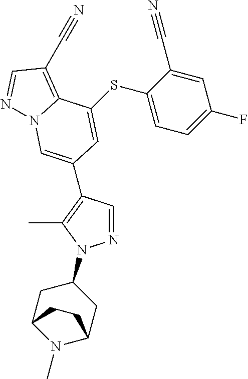 [SMILES: Cc1c(-c2cc(Sc3ccc(F)cc3C#N)c3c(C#N)cnn3c2)cnn1[C@H]1C[C@H]2CC[C@@H](C1)N2C] | 4-((2-cyano-4- fluorophenyl)thio)-6-(5- methyl-1-((1R,3r,5S)-8- methyl-8- azabicyclo[3.2.1]octan-3- yl)-1H-pyrazol-4- yl)pyazolo[1,5- a]pyridine-3-carbonitrile | Example 53 | 498.1 | 1 |
| 301 | 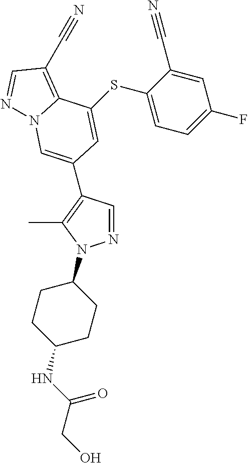 [SMILES: Cc1c(-c2cc(Sc3ccc(F)cc3C#N)c3c(C#N)cnn3c2)cnn1[C@H]1CC[C@H](NC(=O)CO)CC1] | N-((1r,4r)-4-(4-(3-cyano- 4-((2-cyano-4- fluorophenyl)thio)pyrazolo [1,5-a]pyridin-6-yl)-5- methyl-1H-pyrazol-1- yl)cyclohexyl)-2- hydroxyacetamide | Example 36 | 530.2 | 1 |
| 302 | 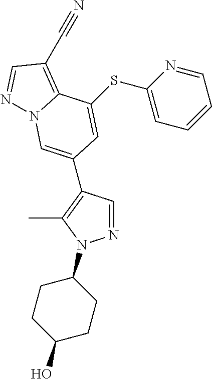 [SMILES: Cc1c(-c2cc(Sc3ccccn3)c3c(C#N)cnn3c2)cnn1[C@H]1CC[C@@H](O)CC1] | 6-(1-((1s,4s)-4- hydroxycyclohexyl)-5- methyl-1H-pyrazol-4-yl)- 4-(pyridin-2- ylthio)pyrazolo∥,5- a]pyridine-3-carbonitrile | Example 53 | 431.3 | 4 |
| 303 | 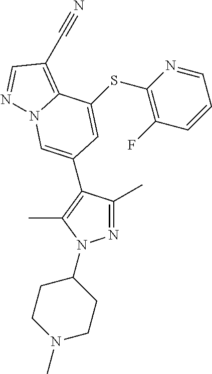 [SMILES: Cc1nn(C2CCN(C)CC2)c(C)c1-c1cc(Sc2ncccc2F)c2c(C#N)cnn2c1] | 6-(3,5-dimethyl-1-(1- methylpiperidin-4-yl)-1H- pyrazol-4-yl)-4-((3- fluoropyridin-2- yl)thio)pyrazolo[1,5- a]pyridine-3-carbontirile | Example 32 | 462.4 | 6 |
| 304 | 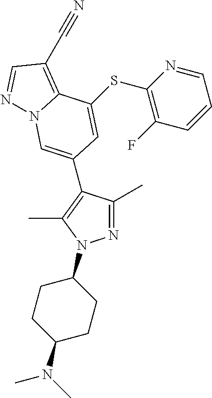 [SMILES: Cc1nn([C@H]2CC[C@@H](N(C)C)CC2)c(C)c1-c1cc(Sc2ncccc2F)c2c(C#N)cnn2c1] | 6-(1-((1s,4s)-4- (dimethylamino)cyclohex- yl)-3,5-dimethyl-1H- pyrazol-4-yl)-4-((3- fluoropyridin-2- yl)thio)pyrazolo[1,5- a]pryidine-3-carbonitrile | Example 32 | 490.3 | 3.5 |
| 305 | 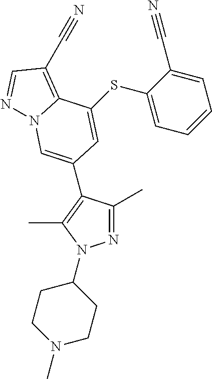 [SMILES: Cc1nn(C2CCN(C)CC2)c(C)c1-c1cc(Sc2ccccc2C#N)c2c(C#N)cnn2c1] | 4-((2-cyanophenyl)thio)- 6-(3,5-dimethyl-1-(1- methylpiperidin-4-yl)-1H- pyrazol-4-yl)pyrazolo[1,5- a]pyridine-3-carbonitrile | Example 32 | 468.3 | 5 |
| 306 | 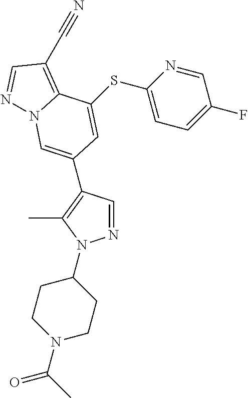 [SMILES: CC(=O)N1CCC(n2ncc(-c3cc(Sc4ccc(F)cn4)c4c(C#N)cnn4c3)c2C)CC1] | 6-(1-(1-acetylpiperidin-4- yl)-5-methyl-1H-pyrazol- 4-yl)-4-((5-fluoropyridin- 2-yl)thio)pyrazolo[1,5- a]pyridine-3-carbonitrile | Example 39 | 476.1 | 6 |
| 307 | 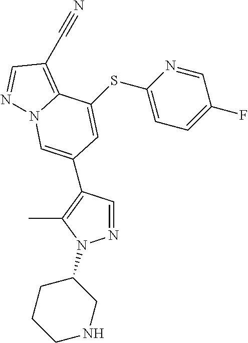 [SMILES: Cc1c(-c2cc(Sc3ccc(F)cn3)c3c(C#N)cnn3c2)cnn1[C@H]1CCCNC1] | (S)-4-((5-fluoropyridin-2- yl)thio)-6-(5-methyl-1- (piperidin-3-yl)-1H- pyrazol-4-yl)pyrazolo[1,5- a]pyridine-3-carbonitrile | Example 53 | 434.2 | 5 |
| 308 | 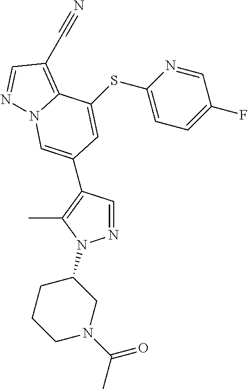 [SMILES: CC(=O)N1CCC[C@H](n2ncc(-c3cc(Sc4ccc(F)cn4)c4c(C#N)cnn4c3)c2C)C1] | (S)-6-(1-(1- acetylpiperidin-3-yl)-5- methyl-1H-pyrazol-4-yl)- 4-((5-fluoropyridin-2- yl)thio)pyrazolo[1,5- a]pyridine-3-carbonitrile | Example 39 | 476.3 | 7 |
| 309 | 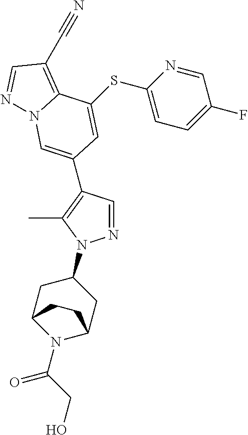 [SMILES: Cc1c(-c2cc(Sc3ccc(F)cn3)c3c(C#N)cnn3c2)cnn1[C@H]1C[C@H]2CC[C@@H](C1)N2C(=O)CO] | 4-((5-fluoropyridin-2- yl)thio)-6-(1-((1R,3r,5S)- 8-(2-hydroxyacetyl)-8- azabicyclo[3.2.1]octan-3- yl)-5-methyl-1H-pyrazol- 4-yl)pyrazolo[1,5- a]pyridine-3-carbonitrile | Example 36 | 518.2 | 6 |
| 310 | 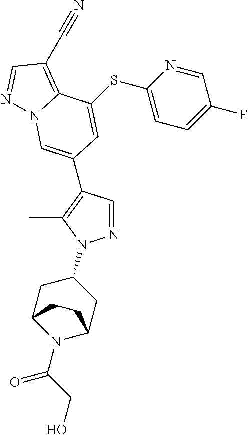 [SMILES: Cc1c(-c2cc(Sc3ccc(F)cn3)c3c(C#N)cnn3c2)cnn1[C@@H]1C[C@H]2CC[C@@H](C1)N2C(=O)CO] | 4-((5-fluoropyridin-2- y)thio)-6-(1-((1R,3s,5S)- 8-(2-hydroxyacetyl)-8- azabicyclo[3.2.1]octan-3- yl)-5-methyl-1H-pyrazol- 4-yl)pyrazolo[1,5- a]pyridine-3-carobnitrile | Example 36 | 518.0 | 8 |
| 311 | 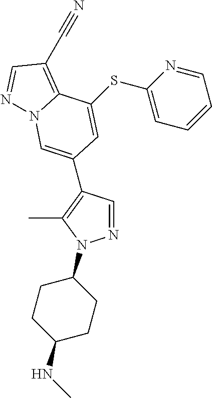 [SMILES: CN[C@H]1CC[C@@H](n2ncc(-c3cc(Sc4ccccn4)c4c(C#N)cnn4c3)c2C)CC1] | 6-(5-methyl-1-((1s,4s)-4- (methylamino)cyclohexyl)- 1H-pyrazol-4-yl)-4- (pyridin-2- ylthio)pyrazolo[1,5- a]pyridine-3-carbonitrile | Example 32 | 444.2 | 2 |
| 312 | 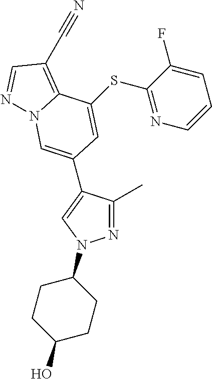 [SMILES: Cc1nn([C@H]2CC[C@@H](O)CC2)cc1-c1cc(Sc2ncccc2F)c2c(C#N)cnn2c1] | 4-((3-fluoropyridin-2- yl)thio)-6-(1-((1s,4s)-4- hydroxycyclohexyl)-3- methyl-1H-pyrazol-4- yl)pyrazolo[1,5- a]pyridine-3-carbonitrile | Example 53 | 449.1 | 1.5 |
| 313 | 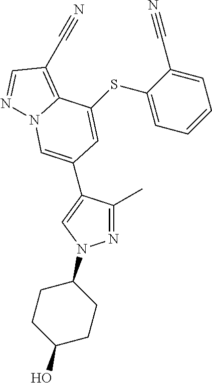 [SMILES: Cc1nn([C@H]2CC[C@@H](O)CC2)cc1-c1cc(Sc2ccccc2C#N)c2c(C#N)cnn2c1] | 4-((2-cyanophenyl)thio)- 6-(1-((1s,4s)-4- hydroxycyclohexyl)-3- methyl-1H-pyrazol-4- yl)pyrazolo[1,5- a]pyridine-3-carbonitrile | Example 53 | 455.1 | 1 |
| 314 | 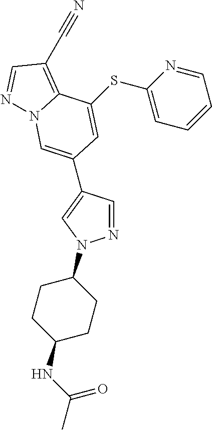 [SMILES: CC(=O)N[C@H]1CC[C@@H](n2cc(-c3cc(Sc4ccccn4)c4c(C#N)cnn4c3)cn2)CC1] | N-((1s,4s)-4-(4-(3-cyano- 4-(pyridin-2- ylthio)pyrazolo[1,5- a]pyridin-6-yl)-1H- pyrazol-1- yl)cyclohexyl)acetamide | Example 39 | 458.2 | 2 |
| 315 | 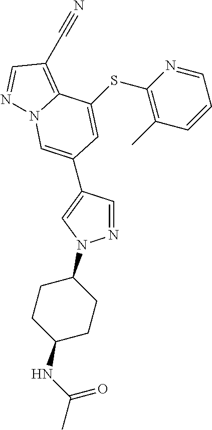 [SMILES: CC(=O)N[C@H]1CC[C@@H](n2cc(-c3cc(Sc4ncccc4C)c4c(C#N)cnn4c3)cn2)CC1] | N-((1s,4s)-4-(4-(3-cyano- 4-((3-methylpyridin-2- yl)thio)pyrazolo[1,5- a]pyridin-6-yl)-1H- pyrazol-1- yl)cyclohexyl)acetamide | Example 39 | 472.3 | 3 |
| 316 | 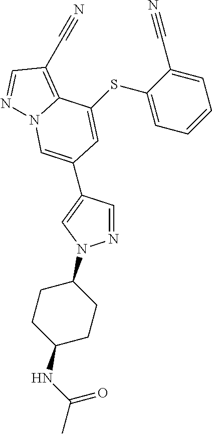 [SMILES: CC(=O)N[C@H]1CC[C@@H](n2cc(-c3cc(Sc4ccccc4C#N)c4c(C#N)cnn4c3)cn2)CC1] | N-((1s,4s)-4-(4-(3-cyano- 4-((2- cyanophenyl)thio)pyrazolo [1,5-a]pyridin-6-yl)-1H- pyrazol-1- yl)cyclohexyl)acetamide | Example 39 | 482.2 | 0.4 |
| 317 | 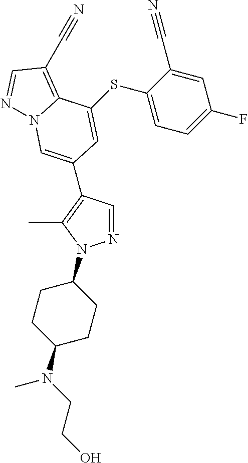 [SMILES: Cc1c(-c2cc(Sc3ccc(F)cc3C#N)c3c(C#N)cnn3c2)cnn1[C@H]1CC[C@@H](N(C)CCO)CC1] | 4-((2-cyano-4- fluorophenyl)thio)-6-(1- ((1s,4s)-4-((2- hydroxyethyl)(methyl)ami- no)cyclohexyl)-5-methyl- 1H-pyrazol-4- yl)pyrazolo[1,5- a]pyridine-3-carbonitrile | Example 35 | 530.3 | 0.5 |
| 318 | 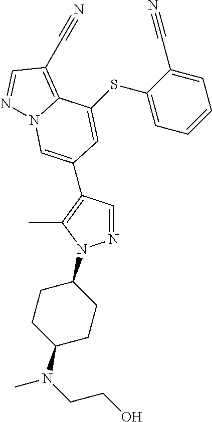 [SMILES: Cc1c(-c2cc(Sc3ccccc3C#N)c3c(C#N)cnn3c2)cnn1[C@H]1CC[C@@H](N(C)CCO)CC1] | 4-((2-cyanophenyl)thio)- 6-(1-((1s,4s)-4-((2- hydroxyethyl)(methyl)ami- no)cyclohexyl)-5-methyl- 1H-pyrazol-4- yl)pyrazolo[1,5- a]pyridine-3-carbonitrile | Example 35 | 512.2 | 0.4 |
| 319 | 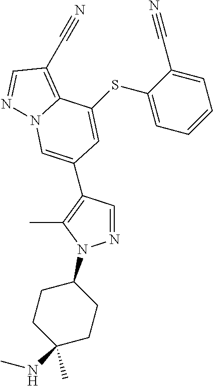 [SMILES: CN[C@]1(C)CC[C@H](n2ncc(-c3cc(Sc4ccccc4C#N)c4c(C#N)cnn4c3)c2C)CC1] | 4-((2-cyanophenyl)thio)- 6-(5-methyl-1-((1s,4s)-4- methyl-4- (methylamino)cyclohexyl)- 1H-pyrazol-4- yl)pyrazolo[1,5- a]pyridine-3-carbonitrile | Example 35 | 482.3 | 0.3 |
| 320 | 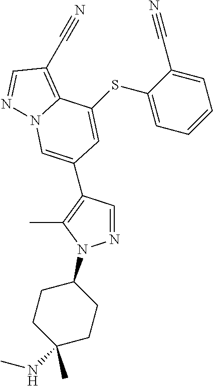 [SMILES: CN[C@]1(C)CC[C@@H](n2ncc(-c3cc(Sc4ccccc4C#N)c4c(C#N)cnn4c3)c2C)CC1] | 4-((2-cyanophenyl)thio)- 6-(5-methyl-1-((1r,4r)-4- methyl-4- (methylamino)cyclohexyl)- 1H-pyrazol-4- yl)pyrazolo[1,5- a]pyridine-3-carbonitirle | Example 35 | 482.2 | 0.3 |
| 321 | 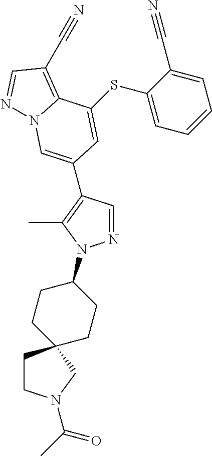 [SMILES: CC(=O)N1CC[C@]2(CC[C@@H](n3ncc(-c4cc(Sc5ccccc5C#N)c5c(C#N)cnn5c4)c3C)CC2)C1] | 6-(1-((5r,8r)-2-acetyl-2- azaspiro[4.5]decan-8-yl)- 5-methyl-1H-pyrazol-4- yl)-4-((2- cyanophenyl)thio)pyrazolo [1,5-a]pyridine-3- carbonitrile | Example 39 | 536.2 | 1 |
| 322 | 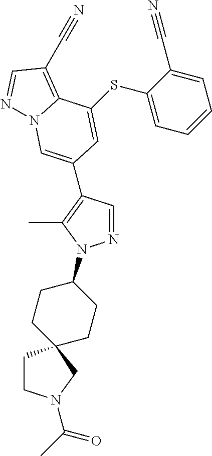 [SMILES: CC(=O)N1CC[C@]2(CC[C@H](n3ncc(-c4cc(Sc5ccccc5C#N)c5c(C#N)cnn5c4)c3C)CC2)C1] | 6-(1-((5s,8s)-2-acetyl-2- azaspiro[4.5]decan-8-yl)- 5-methyl-1H-pyrazol-4- yl)-4-((2- cyanophenyl)thio)pyrazolo [1,5-a]pyridine-3- carbonitrile | Example 39 | 536.3 | 0.6 |
| 323 | 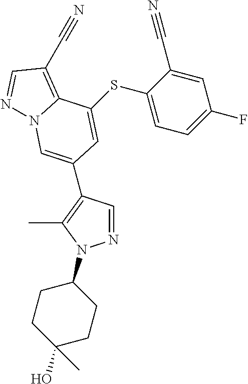 [SMILES: Cc1c(-c2cc(Sc3ccc(F)cc3C#N)c3c(C#N)cnn3c2)cnn1[C@H]1CC[C@](C)(O)CC1] | 4-((2-cyano-4- fluorophenyl)thio)-6-(1- ((1r,4r)-4-hydroxy-4- methylcyclohexyl)-5- methyl-1H-pyrazol-4- yl)pyrazolo[1,5- a]pyridine-3-carbonitrile | Example 54 | 487.3 | 1 |
| 324 | 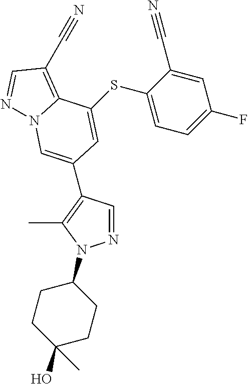 [SMILES: Cc1c(-c2cc(Sc3ccc(F)cc3C#N)c3c(C#N)cnn3c2)cnn1[C@H]1CC[C@@](C)(O)CC1] | 4-((2-cyano-4- fluorophenyl)thio)-6-(1- ((1s,4s)-4-hydroxy-4- methylcyclohexyl)-5- methyl-1H-pyrazol-4- y)pyrazolo[1,5- a]pyridine-3-carbonitrile | Example 54 | 487.1 | 1 |
| 325 | 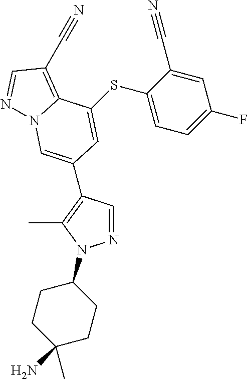 [SMILES: Cc1c(-c2cc(Sc3ccc(F)cc3C#N)c3c(C#N)cnn3c2)cnn1[C@H]1CC[C@@](C)(N)CC1] | 6-(1-((1s,4s)-4-amino-4- methylcyclohexyl)-5- methyl-1H-pyrazol-4-yl)- 4-((2-cyano-4- fluorophenyl)thio)pyrazolo [1,5-a]pyridine-3- carbonitrile | Example 53 | 486.3 | 0.5 |
| 326 | 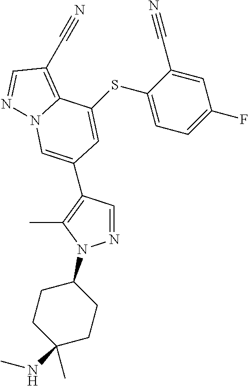 [SMILES: CN[C@]1(C)CC[C@H](n2ncc(-c3cc(Sc4ccc(F)cc4C#N)c4c(C#N)cnn4c3)c2C)CC1] | 4-((2-cyano-4- fluoorphenyl)thio)-6-(5- methyl-1-((1s,4s)-4- methyl-4- (methylamino)cyclohxyl)- 1H-pyrazol-4-e yl)pyrazolo[1,5- a]pyridine-3-carbonitrile | Example 35 | 500.3 | 1 |
| 327 | 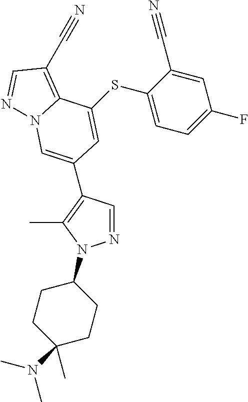 [SMILES: Cc1c(-c2cc(Sc3ccc(F)cc3C#N)c3c(C#N)cnn3c2)cnn1[C@H]1CC[C@@](C)(N(C)C)CC1] | 4-((2-cyano-4- fluorophenyl)thio)-6-(1- ((1s,4s)-4- (dimethylamino)-4- methylcyclohexyl)-5- methyl-1H-pyrazol-4- yl)pyrazolo[1,5- a]pyridine-3-carbonitrile | Example 35 | 514.2 | 0.5 |
| 328 | 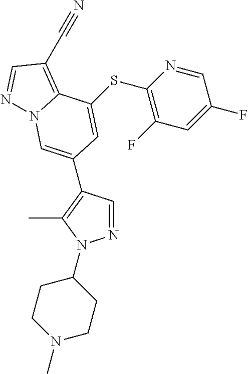 [SMILES: Cc1c(-c2cc(Sc3ncc(F)cc3F)c3c(C#N)cnn3c2)cnn1C1CCN(C)CC1] | 4-((3,5-difluoropyridin-2- yl)thio)-6-(5-methyl-1-(1- methylpiperidin-4-yl)-1H- pyrazol-4-yl)pyrazolo[1,5- a]pyridine-3-carbonitrile | Example 32 | 466.2 | 3 |
| 329 | 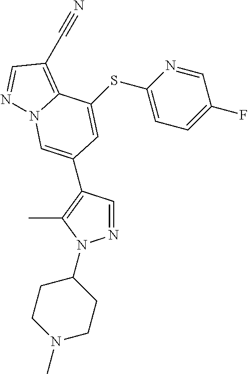 [SMILES: Cc1c(-c2cc(Sc3ccc(F)cn3)c3c(C#N)cnn3c2)cnn1C1CCN(C)CC1] | 4-((5-fluoropyridin-2- yl)thio)-6-(5-methyl-1-(1- methylpiperidin-4-yl)-1H- pyrazol-4-yl)pyrazolo[1,5- a]pyridine-3-carbonitrile | Example 32 | 448.2 | 5 |
| 330 | 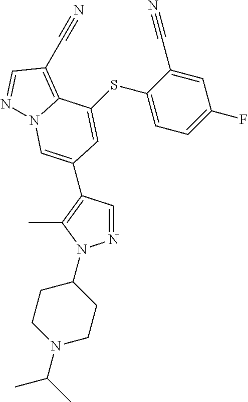 [SMILES: Cc1c(-c2cc(Sc3ccc(F)cc3C#N)c3c(C#N)cnn3c2)cnn1C1CCN(C(C)C)CC1] | 4-((2-cyano-4- fluorophenyl)thio)-6-(1- (1-isopropylpiperidin-4- yl)-5-methyl-1H-pyrazol- 4-yl)pyrazolo[1,5- a]pyridine-3-carbonitrile | Example 32 | 500.2 | 1 |
| 331 | 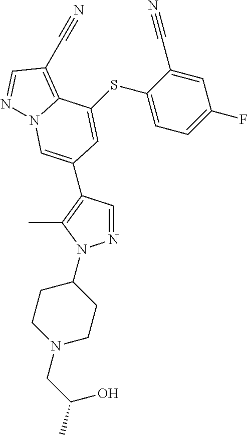 [SMILES: Cc1c(-c2cc(Sc3ccc(F)cc3C#N)c3c(C#N)cnn3c2)cnn1C1CCN(C[C@@H](C)O)CC1] | (R)-4-((2-cyano-4- fluorophenyl)thio)-6-(1- (1-(2- hydroxypropyl)piperidin- 4-yl)-5-methyl-1H- pyrazol-4-yl)pyrazolo[1,5- a]pyridine-3-carbonitrile | Example 35 | 516.2 | 1 |
| 332 | 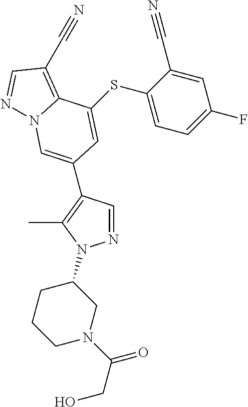 [SMILES: Cc1c(-c2cc(Sc3ccc(F)cc3C#N)c3c(C#N)cnn3c2)cnn1[C@H]1CCCN(C(=O)CO)C1] | (S)-4-((2-cyano-4- fluorophenyl)thio)-6-(1- (1-(2- hydroxyacetyl)piperidin- 3-yl)-5-methyl-1H- pyrazol-4-yl)pyrazolo[1,5- a]pyridine-3-carbonitrile | Example 30 | 516.1 | 1 |
| 333 | 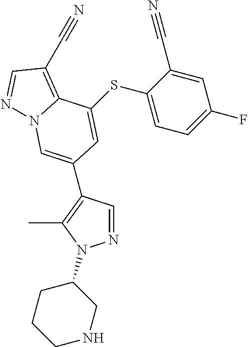 [SMILES: Cc1c(-c2cc(Sc3ccc(F)cc3C#N)c3c(C#N)cnn3c2)cnn1[C@H]1CCCNC1] | (S)-4-((2-cyano-4- fluorophenyl)thio)-6-(5- methyl-1-(piperidin-3-yl)- 1H-pyrazol-4- yl)pyrazolo[1,5- a]pyridine-3-carbonitrile | Exmaple 53 | 458.3 | 1 |
| 334 | 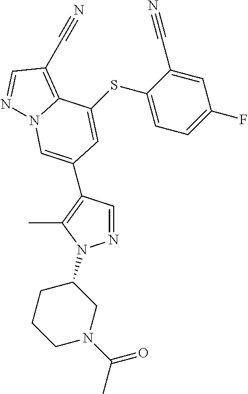 [SMILES: CC(=O)N1CCC[C@H](n2ncc(-c3cc(Sc4ccc(F)cc4C#N)c4c(C#N)cnn4c3)c2C)C1] | (S)-6-(1-(1- acetylpiperidin-3-yl)-5- methyl-1H-pyrazol-4-yl)- 4-((2-cyano-4- fluorophenyl)thio)pyrazolo [1,5-a]pyridine-3- carbonitrile | Example 39 | 500.2 | 1 |
| 335 | 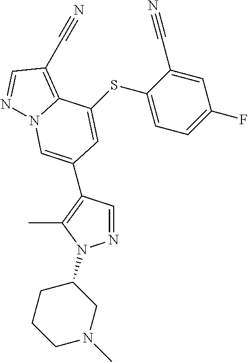 [SMILES: Cc1c(-c2cc(Sc3ccc(F)cc3C#N)c3c(C#N)cnn3c2)cnn1[C@H]1CCCN(C)C1] | (S)-4-((2-cyano-4- fluorophenyl)thio)-6-(5- methyl-1-(1- methylpiperidin-3-yl)-1H- pyrazol-4-yl)pyrazolo[1,5- a]pyridine-3-carbonitrile | Example 53 | 472.1 | 1 |
| 336 | 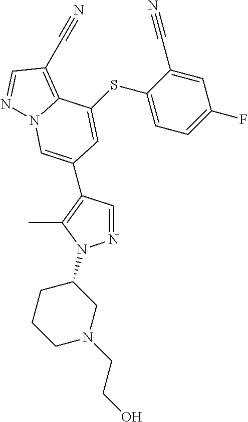 [SMILES: Cc1c(-c2cc(Sc3ccc(F)cc3C#N)c3c(C#N)cnn3c2)cnn1[C@H]1CCCN(CCO)C1] | (S)-4-((2-cyano-4- fluorophenyl)thio)-6-(1- (1-(2- hydroxyethyl)piperidin-3- yl)-5-methyl-1H-pyrazol- 4-yl)pyrazolo[1,5- a]pyridine-3-carbonitrile | Example 35 | 476.2 | 1 |
| 337 | 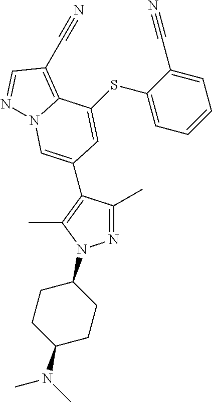 [SMILES: Cc1nn([C@H]2CC[C@@H](N(C)C)CC2)c(C)c1-c1cc(Sc2ccccc2C#N)c2c(C#N)cnn2c1] | 4-((2-cyanophenyl)thio)- 6-(1-((1s,4s)-4- (dimethylamino)cyclohex- yl)-3,5-dimethyl-1H- pyrazol-4-yl)pyrazolo[1,5- a]pyridine-3-carbonitrile | Example 35 | 496.4 | 3 |
| 338 | 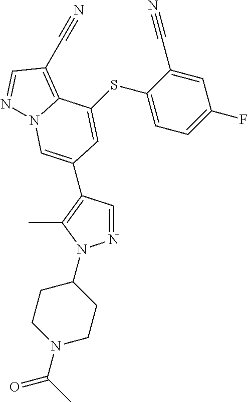 [SMILES: CC(=O)N1CCC(n2ncc(-c3cc(Sc4ccc(F)cc4C#N)c4c(C#N)cnn4c3)c2C)CC1] | 6-(1-(1-acetylpiperidin-4- yl)-5-methyl-1H-pyrazol- 4-yl)-4-((2-cyano-4- fluorophenyl)thio)pyrazolo [1,5-a]pyridine-3- carbonitrile | Example 39 | 500.1 | 1 |
| 339 | 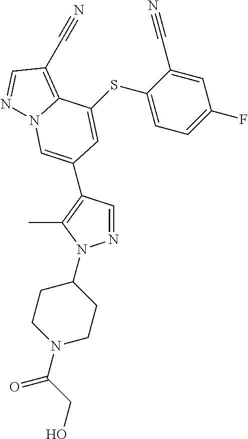 [SMILES: Cc1c(-c2cc(Sc3ccc(F)cc3C#N)c3c(C#N)cnn3c2)cnn1C1CCN(C(=O)CO)CC1] | 4-((2-cyano-4- fluorophenyl)thio)-6-(1- (1-(2- hydroxyacetyl)piperidin- 4-yl)-5-methyl-1H- pyrazol-4-yl)pyrazolo[1,5- a]pyridine-3-carbonitrile | Example 36 | 516.1 | 1 |
| 340 | 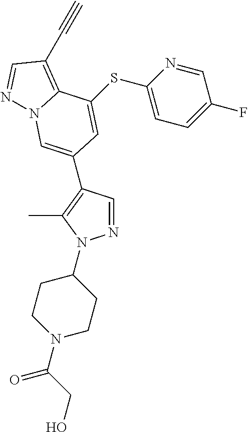 [SMILES: C#Cc1cnn2cc(-c3cnn(C4CCN(C(=O)CO)CC4)c3C)cc(Sc3ccc(F)cn3)c12] | 1-(4-(4-(3-ethynyl-4-((5- fluoropyridin-2- yl)thio)pyrazolo[1,5- a]pyridin-6-yl)-5-methyl- 1H-pyrazol-1- yl)piperidin-1-yl)-2- hydroxyethan-1-one | Example 36 | 492.1 | 7.5 |
| 341 | 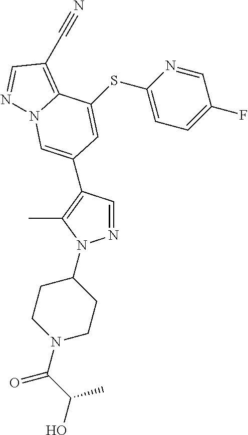 [SMILES: Cc1c(-c2cc(Sc3ccc(F)cn3)c3c(C#N)cnn3c2)cnn1C1CCN(C(=O)[C@H](C)O)CC1] | (S)-4-((5-fluoropyridin-2- yl)thio)-6-(1-(1-(2- hydroxypropanoyl)piperidin- 4-yl)-5-methyl-1H- pyrazol-4-yl)pyrazolo[1,5- a]pyridine-3-carbonitrile | Example 36 | 506.2 | 9 |
| 342 | 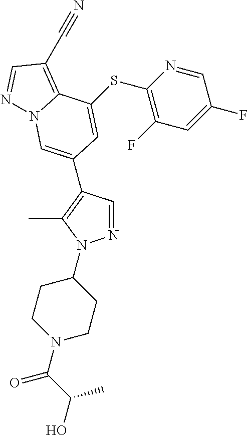 [SMILES: Cc1c(-c2cc(Sc3ncc(F)cc3F)c3c(C#N)cnn3c2)cnn1C1CCN(C(=O)[C@H](C)O)CC1] | (S)-4-((3,5- difluoropyridin-2-yl)thio)- 6-(1-(1-(2- hydroxypropanoyl)piperidin- 4-yl)-5-methyl-1H- pyrazol-4-yl)pyrazolo[1,5- a]pyridine-3-carbonitrile | Example 36 | 524.0 | 6 |
| 343 | 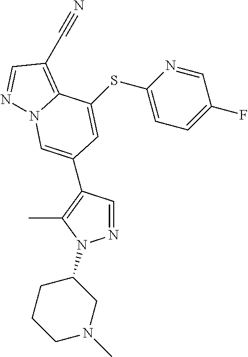 [SMILES: Cc1c(-c2cc(Sc3ccc(F)cn3)c3c(C#N)cnn3c2)cnn1[C@H]1CCCN(C)C1] | (S)-4-((5-fluoropyridin-2- yl)thio)-6-(5-methyl-1-(1- methylpiperidin-3-yl)-1H- pyrazol-4-yl)pyrazolo[1,5- a]pyridine-3-carbonitrile | Example 53 | 448.1 | 9 |
| 344 | 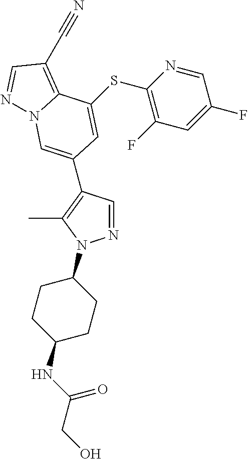 [SMILES: Cc1c(-c2cc(Sc3ncc(F)cc3F)c3c(C#N)cnn3c2)cnn1[C@H]1CC[C@@H](NC(=O)CO)CC1] | N-((1s,4s)-4-(4-(3-cyano- 4-((3,5-difluoropyridin-2- yl)thio)pyrazolo[1,5- a]pyridin-6-yl)-5-methyl- 1H-pyrazol-1- yl)cyclohexyl)-2- hydroxyacetamide | Example 36 | 524.2 | 3 |
| 345 | 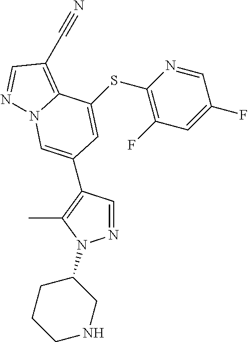 [SMILES: Cc1c(-c2cc(Sc3ncc(F)cc3F)c3c(C#N)cnn3c2)cnn1[C@H]1CCCNC1] | (S)-4-((3,5- difluoropyridin-2-yl)thio)- 6-(5-methyl-1-(piperidin- 3-yl)-1H-pyrazol-4- yl)pyrazolo[1,5- a]pyridine-3-carbonitrile | Example 53 | 452.1 | 4 |
| 346 | 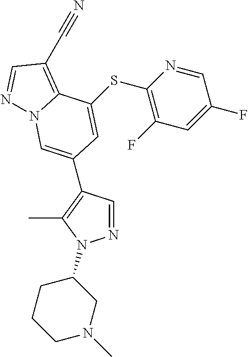 [SMILES: Cc1c(-c2cc(Sc3ncc(F)cc3F)c3c(C#N)cnn3c2)cnn1[C@H]1CCCN(C)C1] | (S)-4-((3,5- difluoropyridin-2-yl)thio)- 6-(5-methyl-1-(1- methylpiperidin-3-yl)-1H- pyrazol-4-yl)pyrazolo[1,5- a]pyridine-3-carbonitrile | Example 53 | 466.1 | 5 |
| 347 | 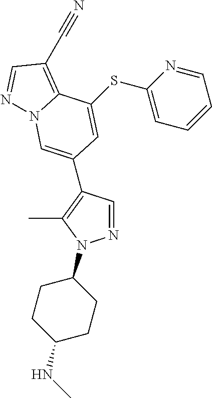 [SMILES: CN[C@H]1CC[C@H](n2ncc(-c3cc(Sc4ccccn4)c4c(C#N)cnn4c3)c2C)CC1] | 6-(5-methyl-1-((1r,4r)-4- (methylamino)cyclohexyl)- 1H-pyrazol-4-yl)-4- (pyridin-2- ylthio)pyrazolo[1,5- a]pyridine-3-carbonitrile | Example 53 | 444.3 | 2.5 |
| 348 | 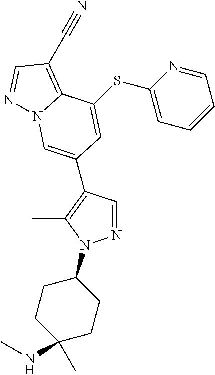 [SMILES: CN[C@]1(C)CC[C@H](n2ncc(-c3cc(Sc4ccccn4)c4c(C#N)cnn4c3)c2C)CC1] | 6-(5-methyl-1-((1s,4s)-4- methyl-4- (methylamino)cyclohexyl)- 1H-pyrazol-4-yl)-4- (pyridin-2- ylthio)pyrazolo[1,5- a]pyridine-3-carbonitrile | Example 35 | 458.2 | 2 |
| 349 | 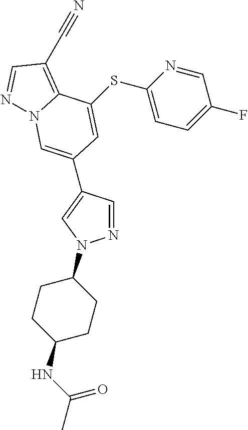 [SMILES: CC(=O)N[C@H]1CC[C@@H](n2cc(-c3cc(Sc4ccc(F)cn4)c4c(C#N)cnn4c3)cn2)CC1] | N-((1s,4s)-4-(4-(3-cyano- 4-((5-fluoropyridin-2- yl)thio)pyrazolo[1,5- a]pyridin-6-yl)-1H- pyrazol-1- yl)cyclohexyl)acetamide | Example 39 | 476.2 | 3 |
| 350 | 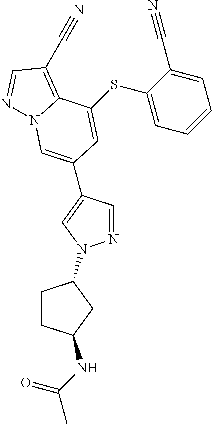 [SMILES: CC(=O)N[C@H]1CC[C@H](n2cc(-c3cc(Sc4ccccc4C#N)c4c(C#N)cnn4c3)cn2)C1] | N-((1s,3S)-3-(4-(3-cyano- 4-((2- cyanophenyl)thio)pyrazolo [1,5-a]pyridin-6-yl)-1H- pyrazol-1- yl)cyclopentyl)acetamide | Example 39 | 468.2 | 0.5 |
| 351 | 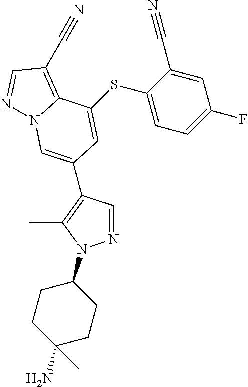 [SMILES: Cc1c(-c2cc(Sc3ccc(F)cc3C#N)c3c(C#N)cnn3c2)cnn1[C@H]1CC[C@](C)(N)CC1] | 6-(1-((1r,4r)-4-amino-4- methylcyclohexyl)-5- methyl-1H-pyrazol-4-yl)- 4-((2-cyano-4- fluorophenyl)thio)pyrazolo [1,5-a]pyridine-3- carbonitrile | Example 53 | 486.1 | 0.5 |
| 352 | 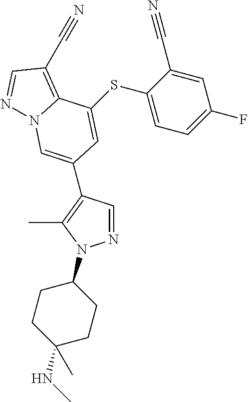 [SMILES: CN[C@]1(C)CC[C@@H](n2ncc(-c3cc(Sc4ccc(F)cc4C#N)c4c(C#N)cnn4c3)c2C)CC1] | 4-((2-cyano-4- fluorophenyl)thio)-6-(5- methyl-1-((1r,4r)-4- methyl-4- (methylamino)cyclohexyl)- 1H-pyrazol-4- y)pyrazolo[1,5- a]pyridine-3-carbonitrile | Example 53 | 500.1 | 0.6 |
| 353 | 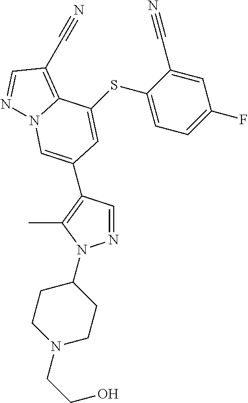 [SMILES: Cc1c(-c2cc(Sc3ccc(F)cc3C#N)c3c(C#N)cnn3c2)cnn1C1CCN(CCO)CC1] | 4-((2-cyano-4- fluorophenyl)thio)-6-(1- (1-(2- hydroxyethyl)piperidin-4- yl)-5-methyl-1H-pyrazol- 4-yl)pyrazolo[1,5- a]pyridine-3-carbonitrile | Example 35 | 502.3 | 1 |
| 354 | 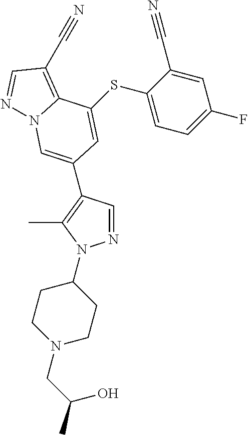 [SMILES: Cc1c(-c2cc(Sc3ccc(F)cc3C#N)c3c(C#N)cnn3c2)cnn1C1CCN(C[C@H](C)O)CC1] | (S)-4-((2-cyano-4- fluorophenyl)thio)-6-(1- (1-(2- hydroxypropyl)piperidin- 4-yl)-5-methyl-1H- pyrazol-4-yl)pyrazolo[1,5- a]pyridine-3-carbonitrile | Example 35 | 516.2 | 2.5 |
| 355 | 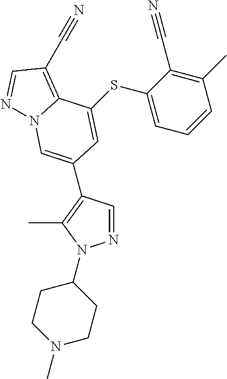 [SMILES: Cc1cccc(Sc2cc(-c3cnn(C4CCN(C)CC4)c3C)cn3ncc(C#N)c23)c1C#N] | 4-((2-cyano-3- methylphenyl)thio)-6-(5- methyl-1-(1- methylpiperidin-4-yl)-1H- pyrazol-4-yl)pyrazolo[1,5- a]pyridine-3-carbonitrile | Example 35 | 468.2 | 2 |
| 356 | 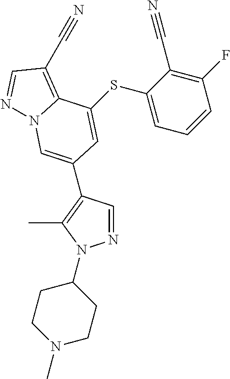 [SMILES: Cc1c(-c2cc(Sc3cccc(F)c3C#N)c3c(C#N)cnn3c2)cnn1C1CCN(C)CC1] | 4-((2-cyano-3- fluorophenyl)thio)-6-(5- methyl-1-(1- methylpiperidin-4-yl)-1H- pyrazol-4-yl)pyrazolo[1,5- a]pyridine-3-carbonitrile | Example 35 | 472.2 | 10 |
| 357 | 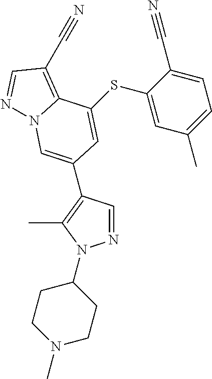 [SMILES: Cc1ccc(C#N)c(Sc2cc(-c3cnn(C4CCN(C)CC4)c3C)cn3ncc(C#N)c23)c1] | 4-((2-cyano-5- methylphenyl)thio)-6-(5- methyl-1-(1- methylpiperidin-4-yl)-1H- pyrazol-4-yl)pyrazolo[1,5- a]pyridine-3-carbonitrile | Example 35 | 468.2 | 2 |
| 358 | 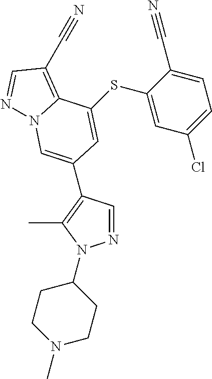 [SMILES: Cc1c(-c2cc(Sc3cc(Cl)ccc3C#N)c3c(C#N)cnn3c2)cnn1C1CCN(C)CC1] | 4-((5-chloro-2- cyanophenyl)thio)-6-(5- methyl-1-(1- methylpiperidin-4-yl)-1H- pyrazol-4-yl)pyrazolo[1,5- a]pyridine-3-carbonitrile | Example 53 | 488.0 | 2 |
| 359 | 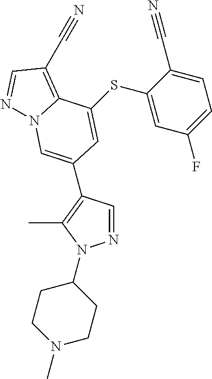 [SMILES: Cc1c(-c2cc(Sc3cc(F)ccc3C#N)c3c(C#N)cnn3c2)cnn1C1CCN(C)CC1] | 4-((2-cyano-5- fluorophenyl)thio)-6-(5- methyl-1-(1- methylpiperidin-4-yl)-1H- pyrazol-4-yl)pyrazolo[1,5- a]pyridine-3-carbonitrile | Example 53 | 472.1 | 1.5 |
| 360 | 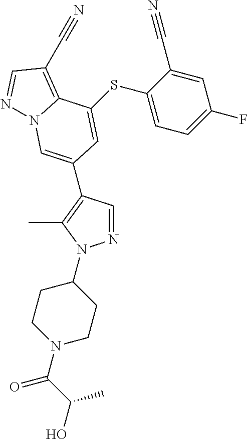 [SMILES: Cc1c(-c2cc(Sc3ccc(F)cc3C#N)c3c(C#N)cnn3c2)cnn1C1CCN(C(=O)[C@H](C)O)CC1] | (S)-4-((2-cyano-4- fluorophenyl)thio)-6-(1- (1-(2- hydroxypropanoyl)piperidin- 4-yl)-5-methyl-1H- pyrazol-4-yl)pyrazolo[1,5- a]pyridine-3-carbonitrile | Example 36 | 530.3 | 2 |
| 361 | 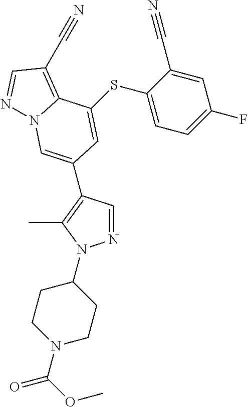 [SMILES: COC(=O)N1CCC(n2ncc(-c3cc(Sc4ccc(F)cc4C#N)c4c(C#N)cnn4c3)c2C)CC1] | methyl 4-(4-(3-cyano-4- ((2-cyano-4- fluorophenyl)thio)pyrazolo [1,5-a]pyridin-6-yl)-5- methyl-1H-pyrazol-1- yl)piperidine-1- carboxylate | Example 36 | 516.3 | 2 |
| 362 | 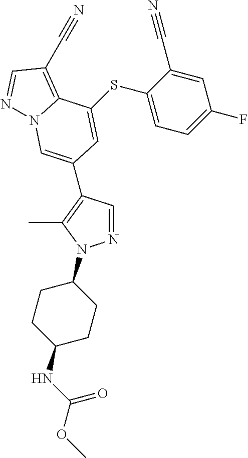 [SMILES: COC(=O)N[C@H]1CC[C@@H](n2ncc(-c3cc(Sc4ccc(F)cc4C#N)c4c(C#N)cnn4c3)c2C)CC1] | methyl ((1s,4s)-4-(4-(3- cyano-4-((2-cyano-4- fluorophenyl)thio)pyrazolo [1,5-a]pyridin-6-yl)-5- methyl-1H-pyrazol-1- yl)cyclohexyl)carbamate | Example 36 | 530.2 | 1 |
| 363 | 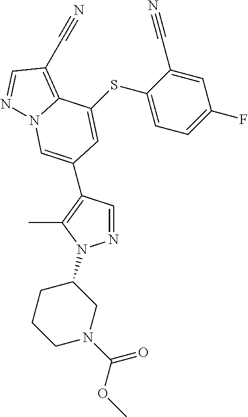 [SMILES: COC(=O)N1CCC[C@H](n2ncc(-c3cc(Sc4ccc(F)cc4C#N)c4c(C#N)cnn4c3)c2C)C1] | methyl (S)-3-(4-(3-cyano- 4-((2-cyano-4- fluorophenyl)thio)pyrazolo [1,5-a]pyridin-6-yl)-5- methyl-1H-pyrazol-1- yl)piperidine-1- carboxylate | Example 36 | 516.1 | 2 |
| 364 | 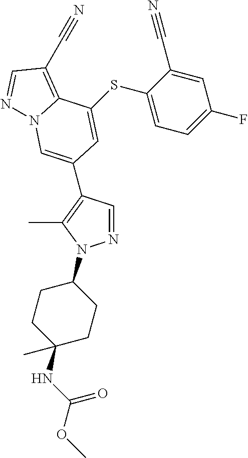 [SMILES: COC(=O)N[C@]1(C)CC[C@H](n2ncc(-c3cc(Sc4ccc(F)cc4C#N)c4c(C#N)cnn4c3)c2C)CC1] | methyl ((1s,4s)-4-(4-(3- cyano-4-((2-cyano-4- fluorophenyl)thio)pyrazolo [1,5-a]pyridin-6-yl)-5- methyl-1H-pyrazol-1-yl)- 1- methylcyclohexyl)carba- mate | Example 36 | 544.3 | 3 |
| 365 | 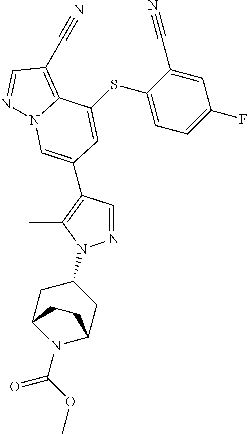 [SMILES: COC(=O)N1[C@@H]2CC[C@H]1C[C@H](n1ncc(-c3cc(Sc4ccc(F)cc4C#N)c4c(C#N)cnn4c3)c1C)C2] | methyl (1R,3s,5S)-3-(4- (3-cyano-4-((2-cyano-4- fluorophenyl)thio)pyrazolo [1,5-a]pyridin-6-yl)-5- methyl-1H-pyrazol-1-yl)- 8-azabicyclo[3.2.1]octane- 8-carboxylate | Example 36 | 542.2 | 2 |
| 366 | 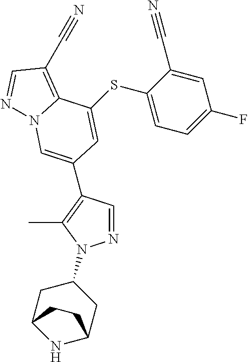 [SMILES: Cc1c(-c2cc(Sc3ccc(F)cc3C#N)c3c(C#N)cnn3c2)cnn1[C@@H]1C[C@H]2CC[C@@H](C1)N2] | 6-(1-((1R,3s,5S)-8- azabicyclo[3.2.1]octan-3- yl)-5-methyl-1H-pyrazol- 4-yl)-4-((2-cyano-4- fluorophenyl)thio)pyrazolo [1,5-a]pyridine-3- carbonitrile | Example 53 | 484.2 | 0.6 |
| 367 | 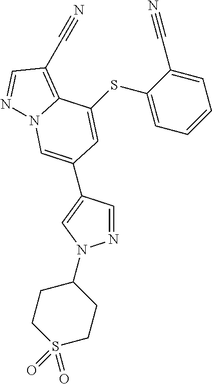 [SMILES: N#Cc1ccccc1Sc1cc(-c2cnn(C3CCS(=O)(=O)CC3)c2)cn2ncc(C#N)c12] | 4-((2-cyanophenyl)thio)- 6-(1-(1,1- dioxidotetrahydro-2H- thiopyran-4-yl)-1H- pyrazol-4-yl)pyrazolo[1,5- a]pyridine-3-carbonitrile | Example 53 | 475.1 | 0.4 |
| 368 | 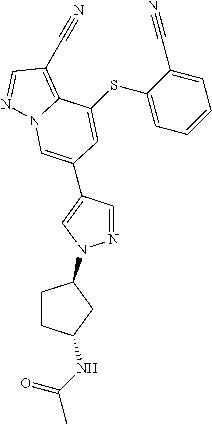 [SMILES: CC(=O)N[C@@H]1CC[C@@H](n2cc(-c3cc(Sc4ccccc4C#N)c4c(C#N)cnn4c3)cn2)C1] | N-((1R,3R)-3-(4-(3- cyano-4-((2- cyanophenyl)thio)pyrazolo [1,5-a]pyridin-6-yl)-1H- pyrazol-1- yl)cyclopentyl)acetamide | Example 36 | 468.2 | 0.4 |
| 369 | 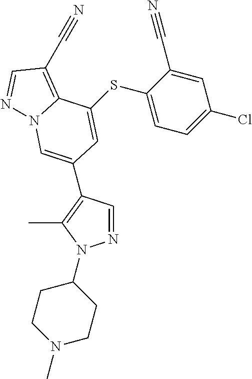 [SMILES: Cc1c(-c2cc(Sc3ccc(Cl)cc3C#N)c3c(C#N)cnn3c2)cnn1C1CCN(C)CC1] | 4-((4-chloro-2- cyanophenyl)thio)-6-(5- methyl-1-(1- methylpiperidin-4-yl)-1H- pyrazol-4-yl)pyrazolo[1,5- a]pyridine-3-carbonitrile | Example 53 | 488.2 | 5 |
| 370 | 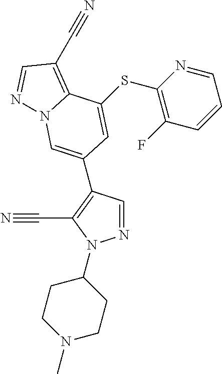 [SMILES: CN1CCC(n2ncc(-c3cc(Sc4ncccc4F)c4c(C#N)cnn4c3)c2C#N)CC1] | 6-(5-cyano-1-(1- methylpiperidin-4-yl)-1H- pyrazol-4-yl)-4-((3- fluoropyridin-2- yl)thio)pyrazolo[1,5- a]pyridine-3-carbonitrile | Example 53 | 459.3 | 35 |
| 371 | 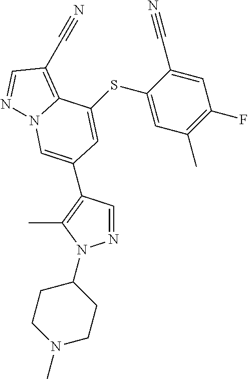 [SMILES: Cc1cc(Sc2cc(-c3cnn(C4CCN(C)CC4)c3C)cn3ncc(C#N)c23)c(C#N)cc1F] | 4-((2-cyano-4-fluoro-5- methylphenyl)thio)-6-(5- methyl-1-(1- methylpiperidin-4-yl)-1H- pyrazol-4-yl)pyrazolo[1,5- a]pyridine-3-carbonitrile | Example 53 | 486.3 | 5 |
| 372 | 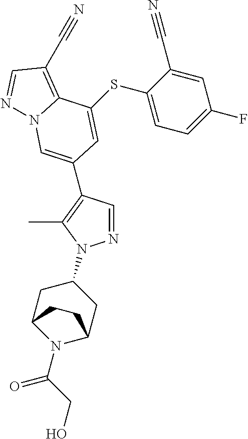 [SMILES: Cc1c(-c2cc(Sc3ccc(F)cc3C#N)c3c(C#N)cnn3c2)cnn1[C@@H]1C[C@H]2CC[C@@H](C1)N2C(=O)CO] | 4-((2-cyano-4- fluorophenyl)thio)-6-(1- ((1R,3s,5S)-8-(2- hydroxyacetyl)-8- azabicyclo[3.2.1]octan-3- yl)-5-methyl-1H-pyrazol- 4-yl)pyrazolo[1,5- a]pyridine-3-carbonitrile | Example 36 | 542.1 | 1 |
| 373 | 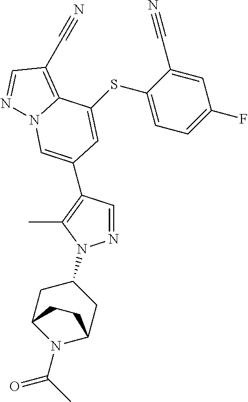 [SMILES: CC(=O)N1[C@@H]2CC[C@H]1C[C@H](n1ncc(-c3cc(Sc4ccc(F)cc4C#N)c4c(C#N)cnn4c3)c1C)C2] | 6-(1-((1R,3s,5S)-8-acetyl- 8-azabicyclo[3.2.1]octan- 3-yl)-5-methyl-1H- pyrazol-4-yl)-4-((2-cyano- 4- fluorophenyl)thio)pyrazolo [1,5-a]pyridine-3- carbonitrile | Eaxmple 39 | 526.4 | 2 |
| 374 | 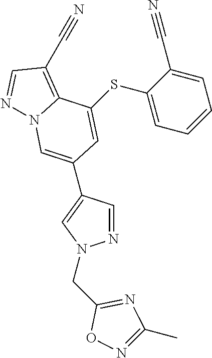 [SMILES: Cc1noc(Cn2cc(-c3cc(Sc4ccccc4C#N)c4c(C#N)cnn4c3)cn2)n1] | 4-((2-cyanophenyl)thio)- 6-(1-((3-methyl-1,2,4- oxadiazol-5-yl)methyl)- 1H-pyrazol-4- yl)pyrazolo[1,5- a]pyridine-3-carbonitrile | Example 35 | 439.1 | 1 |
| 375 | 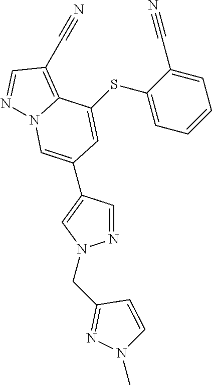 [SMILES: Cn1ccc(Cn2cc(-c3cc(Sc4ccccc4C#N)c4c(C#N)cnn4c3)cn2)n1] | 4-((2-cyanophenyl)thio)- 6-(1-((1-methyl-1H- pyrazol-3-yl)methyl)-1H- pyrazol-4-yl)pyrazolo[1,5- a]pyridine-3-carbonitrile | Example 35 | 437.1 | 0.6 |
| 376 | 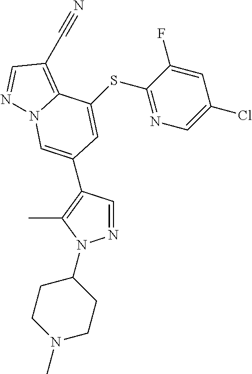 [SMILES: Cc1c(-c2cc(Sc3ncc(Cl)cc3F)c3c(C#N)cnn3c2)cnn1C1CCN(C)CC1] | 4-((5-chloro-3- fluoropyridin-2-yl)thio)-6- (5-methyl-1-(1- methylpiperidin-4-yl)-1H- pyrazol-4-yl)pyrazolo[1,5- a]pryidine-3-carbonitrile | Example 53 | 482.4 | 5.5 |
| 377 | 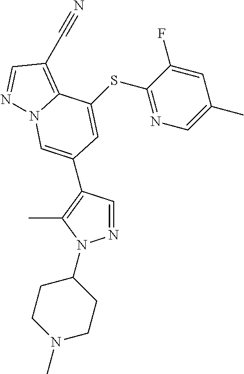 [SMILES: Cc1cnc(Sc2cc(-c3cnn(C4CCN(C)CC4)c3C)cn3ncc(C#N)c23)c(F)c1] | 4-((3-fluoro-5- methylpyridin-2-yl)thio)- 6-(5-methyl-1-(1- methylpiperidin-4-yl)-1H- pyrazol-4-yl)pyrazolo[1,5- a]pyridine-3-carbonitrile | Example 53 | 462.4 | 11 |
| 378 | 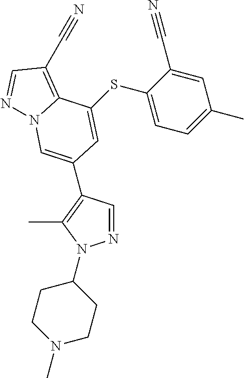 [SMILES: Cc1ccc(Sc2cc(-c3cnn(C4CCN(C)CC4)c3C)cn3ncc(C#N)c23)c(C#N)c1] | 4-((2-cyano-4- methylphenyl)thio)-6-(5- methyl-1-(1- methylpiperidin-4-yl)-1H- pyrazol-4-yl)pyrazolo[1,5- a]pyridine-3-carbonitrile | Example 53 | 468.2 | 4 |
| 379 | 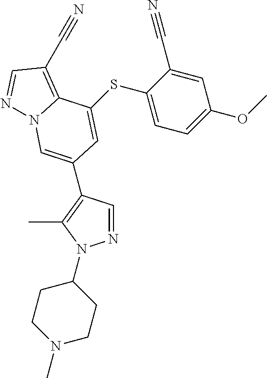 [SMILES: COc1ccc(Sc2cc(-c3cnn(C4CCN(C)CC4)c3C)cn3ncc(C#N)c23)c(C#N)c1] | 4-((2-cyano-4- methoxyphenyl)thio)-6- (5-methyl-1-(1- methylpiperidin-4-yl)-1H- pyrazol-4-yl)pyrazolo[1,5- a]pyridine-3-carbonitrile | Example 53 | 484.2 | 25 |
| 380 | 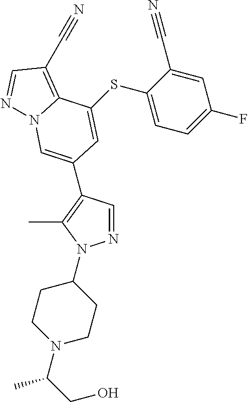 [SMILES: Cc1c(-c2cc(Sc3ccc(F)cc3C#N)c3c(C#N)cnn3c2)cnn1C1CCN([C@@H](C)CO)CC1] | (S)-4-((2-cyano-4- fluorophenyl)thio)-6-(1- (1-(1-hydroxypropan-2- yl)piperidin-4-yl)-5- methyl-1H-pyrazol-4- yl)pyrazolo[1,5- a]pyridine-3-carbonitrile | Example 36 | 516.2 | 1 |
| 381 | 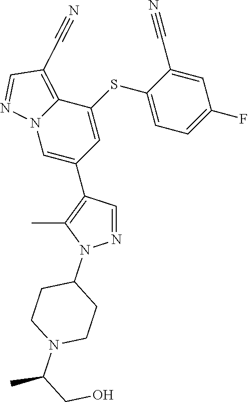 [SMILES: Cc1c(-c2cc(Sc3ccc(F)cc3C#N)c3c(C#N)cnn3c2)cnn1C1CCN([C@H](C)CO)CC1] | (R)-4-((2-cyano-4- fluorophenyl)thio)-6-(1- (1-(1-hydroxypropan-2- yl)piperidin-4-yl)-5- methyl-1H-pyrazol-4- yl)pyrazolo[1,5- a]pyridine-3-carbonitrile | Example 36 | 516.2 | 1 |
| 382 | 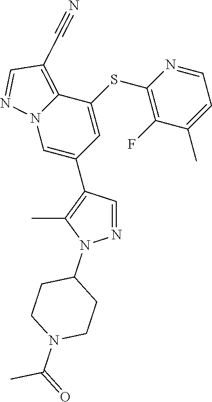 [SMILES: CC(=O)N1CCC(n2ncc(-c3cc(Sc4nccc(C)c4F)c4c(C#N)cnn4c3)c2C)CC1] | 6-(1-(1-acetylpiperidin-4- yl)-5-methyl-1H-pyrazol- 4-yl)-4-((3-fluoro-4- methylpyridin-2- yl)thio)pyrazolo[1,5- a]pyridine-3-carbonitrile | Example 39 | 555.4 | 14 |
| 383 | 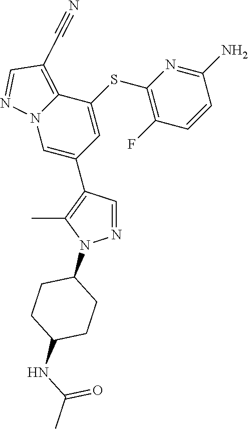 | N-((1s,4s)-4-(4-(4-((6- amino-3-fluoropyridin-2- yl)thio)-3- cyanopyrazolo[1,5- a]pyridin-6-yl)-5-methyl- 1H-pyrazol-1- yl)cyclohexyl)acetamide | Example 39 | 505.3 | 1 |
| 384 | 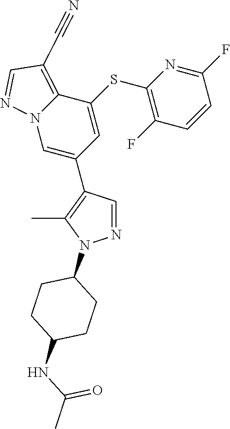 | N-((1s,4s)-4-(4-(3-cyano- 4-((3,6-difluoropyridin-2- y)thio)pyrazolo[1,5- a]pyridin-6-yl)-5-methyl- 1H-pyrazol-1- yl)cyclohexyl)acetamide | Example 39 | 508.1 | 1 |
| 385 | 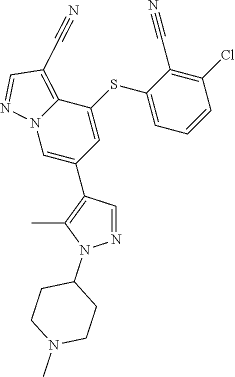 [SMILES: Cc1c(-c2cc(Sc3cccc(Cl)c3C#N)c3c(C#N)cnn3c2)cnn1C1CCN(C)CC1] | 4-((3-chloro-2- cyanophenyl)thio)-6-(5- methyl-1-(1- methylpiperidin-4-yl)-1H- pyrazol-4-yl)pyrazolo[1,5- a]pyridine-3-carbonitrile | Example 53 | 488.1 | 1.5 |
| 386 | 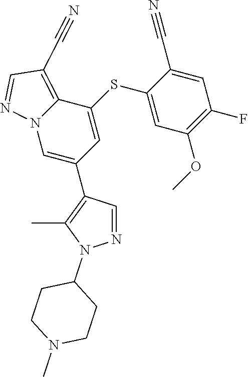 [SMILES: COc1cc(Sc2cc(-c3cnn(C4CCN(C)CC4)c3C)cn3ncc(C#N)c23)c(C#N)cc1F] | 4-((2-cyano-4-fluoro-5- methoxyphenyl)thio)-6- (5-methyl-1-(1- methylpiperidin-4-yl)-1H- pyrazol-4-yl)pyrazolo[1,5- a]pyridine-3-carbonitrile | Example 53 | 502.3 | 5 |
| 387 | 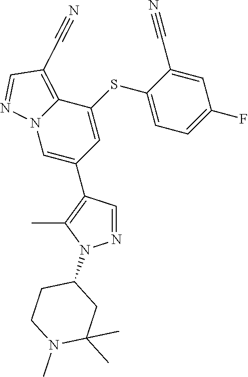 [SMILES: Cc1c(-c2cc(Sc3ccc(F)cc3C#N)c3c(C#N)cnn3c2)cnn1[C@H]1CCN(C)C(C)(C)C1] | (S)-4-((2-cyano-4- fluorophenyl)thio)-6-(5- methyl-1-(1,2,2- trimethylpiperidin-4-yl)- 1H-pyrazol-4- yl)pyrazolo[1,5- a]pyridine-3-carbonitrile | Example 53 | 500.5 | 1 |
| 388 | 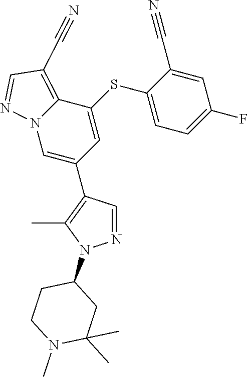 [SMILES: Cc1c(-c2cc(Sc3ccc(F)cc3C#N)c3c(C#N)cnn3c2)cnn1[C@@H]1CCN(C)C(C)(C)C1] | (R)-4-((2-cyano-4- fluorophenyl)thio)-6-(5- methyl-1-(1,2,2- trimethylpiperidin-4-yl)- 1H-pyrazol-4- yl)pyrazolo[1,5- a]pyridine-3-carbonitrile | Example 53 | 500.3 | 0.5 |
| 389 | 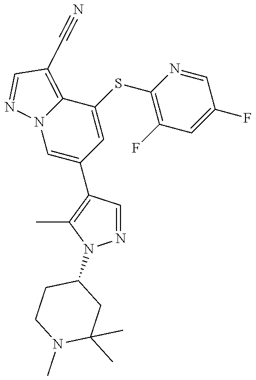 [SMILES: Cc1c(-c2cc(Sc3ncc(F)cc3F)c3c(C#N)cnn3c2)cnn1[C@H]1CCN(C)C(C)(C)C1] | (S)-4-((3,5- difluoropyridin-2-yl)thio)- 6-(5-methyl-1-(1,2,2- trimethylpiperidin-4-yl)- 1H-pyrazol-4- yl)pyrazolo[1,5- a]pyridine-3-carbonitrile | Example 53 | 494.3 | 5 |
| 390 | 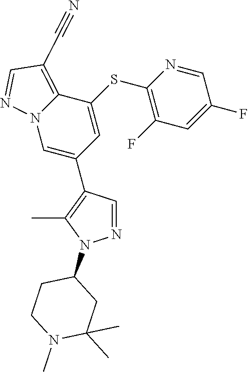 [SMILES: Cc1c(-c2cc(Sc3ncc(F)cc3F)c3c(C#N)cnn3c2)cnn1[C@@H]1CCN(C)C(C)(C)C1] | (R)-4-((3,5- difluoropyridin-2-yl)thio)- 6-(5-methyl-1-(1,2,2- trimethylpiperidin-4-yl)- 1H-pyrazol-4- yl)pyrazolo[1,5- a]pyridine-3-carbonitrile | Example 53 | 494.3 | 42 |
| 391 | 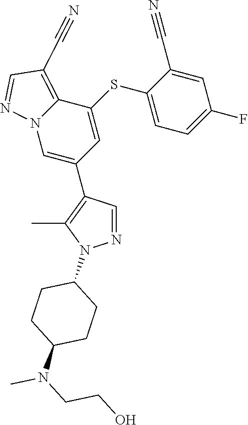 [SMILES: Cc1c(-c2cc(Sc3ccc(F)cc3C#N)c3c(C#N)cnn3c2)cnn1[C@H]1CC[C@H](N(C)CCO)CC1] | 4-((2-cyano-4- fluorophenyl)thio)-6-(1- ((1r,4r)-4-((2- hydroxyethyl)(methyl)ami- no)cyclohexyl)-5-methyl- 1H-pyrazol-4- yl)pyrazolo[1,5- a]pyridine-3-carbonitrile | Example 35 | 530.3 | 1 |
| 392 | 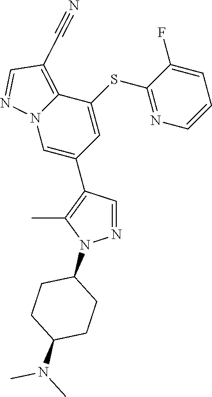 [SMILES: Cc1c(-c2cc(Sc3ncccc3F)c3c(C#N)cnn3c2)cnn1[C@H]1CC[C@@H](N(C)C)CC1] | 6-(1-((1s,4s)-4- (dimethylamino)cyclohex- yl)-1H-1,2,3-triazol-4-yl)- 4-((3-fluoropyridin-2- yl)thio)pyrazolo[1,5- a]pyridine-3-carbonitrile | Example 59 | 463.2 | 12 |
| 393 | 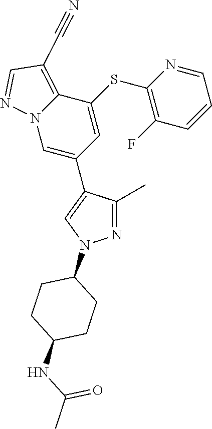 [SMILES: CC(=O)N[C@H]1CC[C@@H](n2cc(-c3cc(Sc4ncccc4F)c4c(C#N)cnn4c3)c(C)n2)CC1] | N-((1s,4s)-4-(4-(3-cyano- 4-((3-fluoropyridin-2- yl)thio)pyrazolo[1,5- a]pyridin-6-yl)-3-methyl- 1H-pyrazol-1- yl)cyclohexyl)acetamide | Example 39 | 490.3 | 6 |
| 394 | 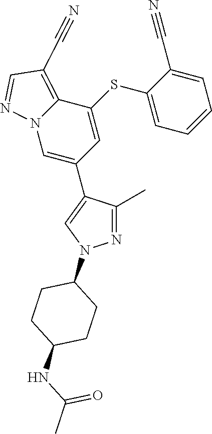 [SMILES: CC(=O)N[C@H]1CC[C@@H](n2cc(-c3cc(Sc4ccccc4C#N)c4c(C#N)cnn4c3)c(C)n2)CC1] | N-((1s,4s)-4-(4-(3-cyano- 4-((2- cyanophenyl)thio)pyrazolo [1,5-a]pyridin-6-yl)-3- methyl-1H-pyrazol-1- yl)cyclohexyl)acetamide | Example 53 | 496.3 | 3 |
| 395 | 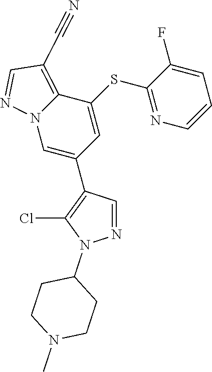 [SMILES: CN1CCC(n2ncc(-c3cc(Sc4ncccc4F)c4c(C#N)cnn4c3)c2Cl)CC1] | 6-(5-chloro-1-(1- methylpiperidin-4-yl)-1H- pyrazol-4-yl)-4-((3- fluoropyridin-2- yl)thio)pyrazolo[1,5- a]pyridine-3-carbonitrile | Example 53 | 468.2 | 3 |
| 396 | 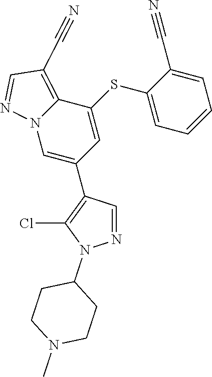 [SMILES: CN1CCC(n2ncc(-c3cc(Sc4ccccc4C#N)c4c(C#N)cnn4c3)c2Cl)CC1] | 6-(5-chloro-1-(1- methylpiperidin-4-yl)-1H- pyrazol-4-yl)-4-((2- cyanophenyl)thio)pyrazolo [1,5-a]pyridine-3- carbonitrile | Example 53 | 474.2 | 1 |
| 397 | 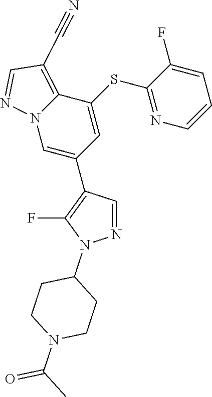 [SMILES: CC(=O)N1CCC(n2ncc(-c3cc(Sc4ncccc4F)c4c(C#N)cnn4c3)c2F)CC1] | 6-(1-(1-acetylpiperidin-4- yl)-5-fluoro-1H-pyrazol- 4-yl)-4-((3-fluoropyridin- 2-yl)thio)pyrazolo[1,5- a]pyridine-3-carbonitrile | Example 53 | 480.3 | 5 |
| 398 | 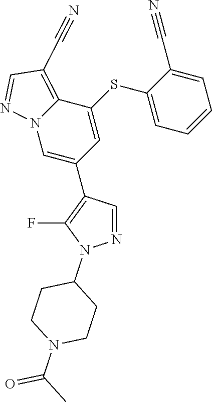 [SMILES: CC(=O)N1CCC(n2ncc(-c3cc(Sc4ccccc4C#N)c4c(C#N)cnn4c3)c2F)CC1] | 6-(1-(1-acetylpiperidin-4- yl)-5-fluoro-1H-pyrazol- 4-yl)-4-((2- cyanophenyl)thio)pyrazolo [1,5-a]pyridine-3- carbonitrile | Example 39 | 486.1 | 2 |
| 399 | 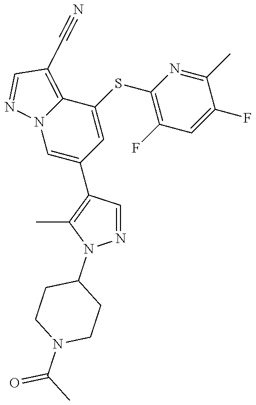 [SMILES: CC(=O)N1CCC(n2ncc(-c3cc(Sc4nc(C)c(F)cc4F)c4c(C#N)cnn4c3)c2C)CC1] | 6-(1-(1-acetylpiperidin-4- yl)-5-methyl-1H-pyrazol- 4-yl)-4-((3,5-difluoro-6- methylpyridin-2- yl)thio)pyrazolo[1,5- a]pyridine-3-carbonitrile | Example 39 | 508.2 | 2 |
| 400 | 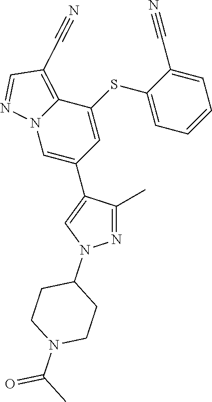 [SMILES: CC(=O)N1CCC(n2cc(-c3cc(Sc4ccccc4C#N)c4c(C#N)cnn4c3)c(C)n2)CC1] | 6-(1-(1-acetylpiperidin-4- yl)-3-methyl-1H-pyrazol- 4-yl)-4-((2- cyanophenyl)thio)pyrazolo [1,5-a]pyridine-3- carbonitrile | Example 39 | 482.2 | 3 |
| 401 | 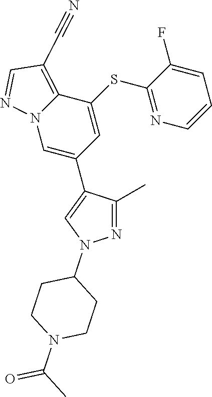 [SMILES: CC(=O)N1CCC(n2cc(-c3cc(Sc4ncccc4F)c4c(C#N)cnn4c3)c(C)n2)CC1] | 6-(1-(1-acetylpiperidin-4- yl)-3-methyl-1H-pyrazol- 4-yl)-4-((3-fluoropyridin- 2-yl)thio)pyrazolo[1,5- a]pyridine-3-carbonitrile | Example 39 | 476.2 | 7 |
| 402 | 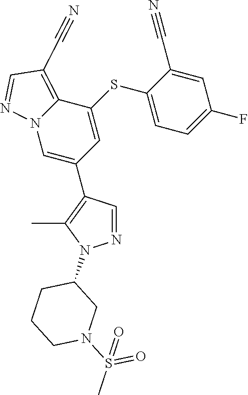 [SMILES: Cc1c(-c2cc(Sc3ccc(F)cc3C#N)c3c(C#N)cnn3c2)cnn1[C@H]1CCCN(S(C)(=O)=O)C1] | (S)-4-((2-cyano-4- fluorophenyl)thio)-6-(5- methyl-1-(1- (methylsulfonyl)piperidin- 3-yl)-1H-pyrazol-4- yl)pyrazolo[1,5- a]pyridine-3-carbonitrile | Example 50 | 536.2 | 2 |
| 403 | 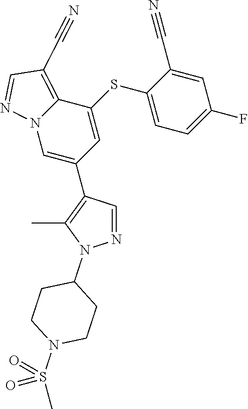 [SMILES: Cc1c(-c2cc(Sc3ccc(F)cc3C#N)c3c(C#N)cnn3c2)cnn1C1CCN(S(C)(=O)=O)CC1] | 4-((2-cyano-4- fluorophenyl)thio)-6-(5- methyl-1-(1- (methylsulfonyl)piperidin- 4-yl)-1H-pyrazol-4- yl)pyrazolo[1,5- a]pyridine-3-carbonitrile | Example 50 | 536.2 | 2 |
| 404 | 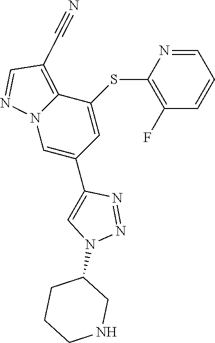 [SMILES: N#Cc1cnn2cc(-c3cn([C@H]4CCCNC4)nn3)cc(Sc3ncccc3F)c12] | (S)-4-((3-fluoropyridin-2- yl)thio)-6-(1-(piperidin-3- yl)-1H-1,2,3-triazol-4- yl)pyrazolo[1,5- a]pyridine-3-carbonitrile | Example 53 | 421.3 | 15 |
| 405 | 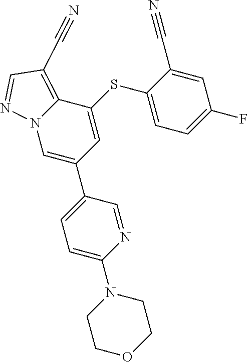 [SMILES: N#Cc1cc(F)ccc1Sc1cc(-c2ccc(N3CCOCC3)nc2)cn2ncc(C#N)c12] | 4-((2-cyano-4- fluorophenyl)thio)-6-(6- morpholinopyridin-3- yl)pyrazolo[1,5- a]pyridine-3-carbonitrile | Example 53 | 457.2 | 2 |
| 406 | 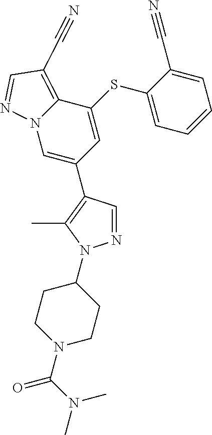 [SMILES: Cc1c(-c2cc(Sc3ccccc3C#N)c3c(C#N)cnn3c2)cnn1C1CCN(C(=O)N(C)C)CC1] | 4-(4-(3-cyano-4-((2- cyanophenyl)thio)pyrazolo [1,5-a]pyridin-6-yl)-5- methyl-1H-pyrazol-1-yl)- N,N-dimethylpiperidine- 1-carboxamide | Example 36 | 511.3 | 1 |
| 407 | 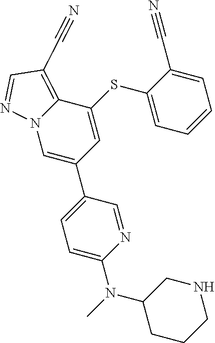 [SMILES: CN(c1ccc(-c2cc(Sc3ccccc3C#N)c3c(C#N)cnn3c2)cn1)C1CCCNC1] | 4-((2-cyanophenyl)thio)- 6-(6-(methyl(piperidin-3- yl)amino)pyridin-3- yl)pyrazolo[1,5- a]pyridine-3-carbonitrile | Example 60 | 466.2 | 0.2 |
| 408 | 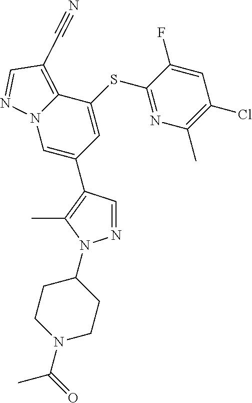 [SMILES: CC(=O)N1CCC(n2ncc(-c3cc(Sc4nc(C)c(Cl)cc4F)c4c(C#N)cnn4c3)c2C)CC1] | 6-(1-(1-acetylpiperidin-4- yl)-5-methyl-1H-pyrazol- 4-yl)-4-((5-chloro-3- fluoro-6-methylpyridin-2- yl)thio)pyrazolo[1,5- a]pyridine-3-carbonitrile | Example 39 | 524.1 | 2 |
| 409 | 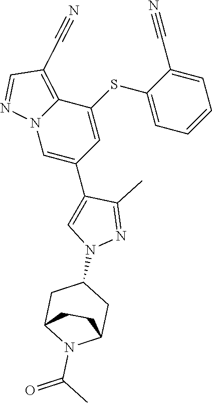 [SMILES: CC(=O)N1[C@@H]2CC[C@H]1C[C@H](n1cc(-c3cc(Sc4ccccc4C#N)c4c(C#N)cnn4c3)c(C)n1)C2] | 6-(1-((1R,3s,5S)-8-acetyl- 8-azabicyclo[3.2.1]octan- 3-yl)-3-methyl-1H- pyrazol-4-yl)-4-((2- cyanophenyl)thio)pyrazolo [1,5-a]pyridine-3- carbonitrile | Example 39 | 508.3 | 5 |
| 410 | 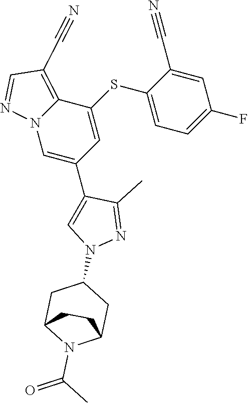 [SMILES: CC(=O)N1[C@@H]2CC[C@H]1C[C@H](n1cc(-c3cc(Sc4ccc(F)cc4C#N)c4c(C#N)cnn4c3)c(C)n1)C2] | 6-(1-((1R,3s,5S)-8-acetyl- 8-azabicyclo[3.2.1]octan- 3-yl)-3-methyl-1H- pyrazol-4-yl)-4-((2-cyano- 4- fluorophenyl)thio)pyrazolo [1,5-a]pyridine-3- carbonitrile | Example 39 | 526.3 | 26 |
| 411 | 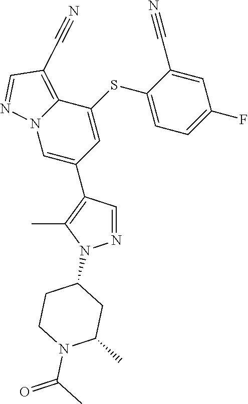 [SMILES: CC(=O)N1CC[C@H](n2ncc(-c3cc(Sc4ccc(F)cc4C#N)c4c(C#N)cnn4c3)c2C)C[C@@H]1C] | 6-(1-((2S,4S)-1-acetyl-2- methylpiperidin-4-yl)-5- methyl-1H-pyrazol-4-yl)- 4-((2-cyano-4- fluorophenyl)thio)pyrazolo [1,5-a]pyridine-3- carbonitrile | Example 39 | 514.3 | 2 |
| 412 | 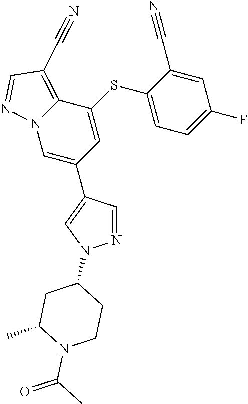 [SMILES: CC(=O)N1CC[C@@H](n2cc(-c3cc(Sc4ccc(F)cc4C#N)c4c(C#N)cnn4c3)cn2)C[C@H]1C] | 6-(1-((2R,4R)-1-acetyl-2- methylpiperidin-4-yl)-1H- pyrazol-4-yl)-4-((2-cyano- 4- fluorophenyl)thio)pyrazolo [1,5-a]pyridine-3- carbonitrile | Example 39 | 500.1 | 1 |
| 413 | 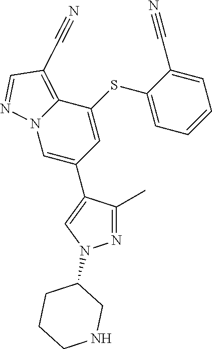 [SMILES: Cc1nn([C@H]2CCCNC2)cc1-c1cc(Sc2ccccc2C#N)c2c(C#N)cnn2c1] | (S)-4-((2- cyanophenyl)thio)-6-(3- methyl-1-(piperidin-3-yl)- 1H-pyrazol-4- yl)pyrazolo[1,5- a]pyridine-3-carbonitrile | Example 53 | 440.1 | 3 |
| 414 | 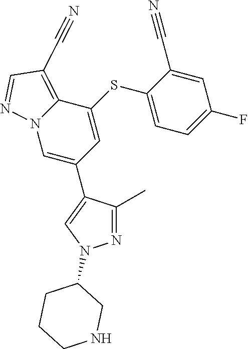 [SMILES: Cc1nn([C@H]2CCCNC2)cc1-c1cc(Sc2ccc(F)cc2C#N)c2c(C#N)cnn2c1] | (S)-4-((2-cyano-4- fluorophenyl)thio)-6-(3- methyl-1-(piperidin-3-yl)- 1H-pyrazol-4- yl)pyrazolo[1,5- a]pyridine-3-carbonitrile | Example 53 | 458.2 | 7 |
| 415 | 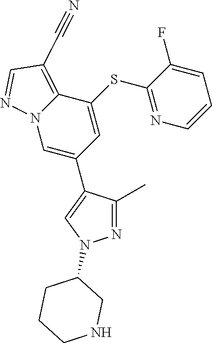 [SMILES: Cc1nn([C@H]2CCCNC2)cc1-c1cc(Sc2ncccc2F)c2c(C#N)cnn2c1] | (S)-4-((3-fluoropyridin-2- yl)thio)-6-(3-methyl-1- (piperidin-3-yl)-1H- pyrazol-4-yl)pyrazolo[1,5- a]pyridine-3-carbonitrile | Example 53 | 434.3 | 7 |
| 416 | 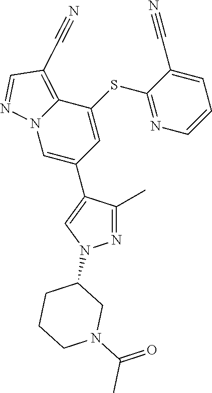 [SMILES: CC(=O)N1CCC[C@H](n2cc(-c3cc(Sc4ncccc4C#N)c4c(C#N)cnn4c3)c(C)n2)C1] | (S)-6-(1-(1- acetylpiperidin-3-yl)-3- methyl-1H-pyrazol-4-yl)- 4-((2- cyanophenyl)thio)pyrazolo [1,5-a]pyridine-3- carbonitrile | Example 39 | 482.4 | 2.5 |
| 417 | 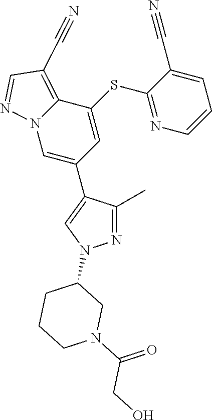 [SMILES: Cc1nn([C@H]2CCCN(C(=O)CO)C2)cc1-c1cc(Sc2ncccc2C#N)c2c(C#N)cnn2c1] | (S)-4-((2- cyanophenyl)thio)-6-(1- (1-(2- hydroxyacetyl)piperidin- 3-yl)-3-methyl-1H- pyrazol-4-yl)pyrazolo[1,5- a]pyridine-3-carbonitrile | Example 30 | 498.2 | 2.5 |
| 418 | 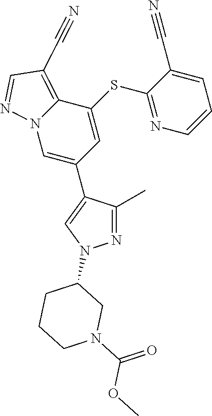 [SMILES: COC(=O)N1CCC[C@H](n2cc(-c3cc(Sc4ncccc4C#N)c4c(C#N)cnn4c3)c(C)n2)C1] | methyl (S)-3-(4-(3-cyano- 4-((2- cyanophenyl)thio)pyrazolo [1,5-a]pyridin-6-yl)-3- methyl-1H-pyrazol-1- yl)piperidine-1- carboxylate | Example 39 | 498.2 | 5 |
| 419 | 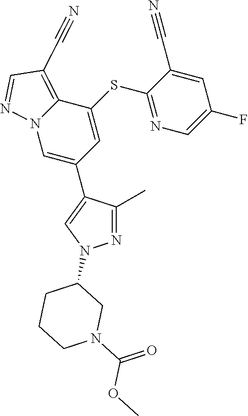 [SMILES: COC(=O)N1CCC[C@H](n2cc(-c3cc(Sc4ncc(F)cc4C#N)c4c(C#N)cnn4c3)c(C)n2)C1] | methyl (S)-3-(4-(3-cyano- 4-((2-cyano-4- fluorophenyl)thio)pyrazolo [1,5-a]pyridin-6-yl)-3- methyl-1H-pyrazol-1- yl)piperidine-1- carboxylate | Example 39 | 516.3 | 11 |
| 420 | 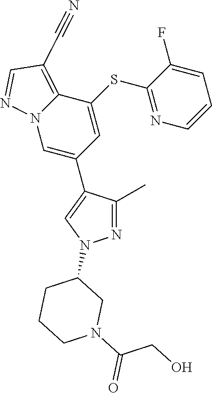 [SMILES: Cc1nn([C@H]2CCCN(C(=O)CO)C2)cc1-c1cc(Sc2ncccc2F)c2c(C#N)cnn2c1] | (S)-4-((3-fluoropyridin-2- yl)thio)-6-(1-(1-(2- hydroxyacetyl)piperidin- 3-yl)-3-methyl-1H- pyrazol-4-yl)pyrazolo[1,5- a]pyridine-3-carbonitrile | Example 30 | 492.1 | 4 |
| 421 | 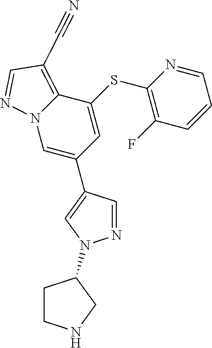 [SMILES: N#Cc1cnn2cc(-c3cnn([C@H]4CCNC4)c3)cc(Sc3ncccc3F)c12] | (S)-4-((3-fluorop;yridin-2- yl)thio)-6-(1-(pyrrolidin- 3-yl)-1H-pyrazol-4- yl)pyrazolo[1,5- a]pyridine-3-carbonitrile | Example 53 | 406.2 | 5 |
| 422 | 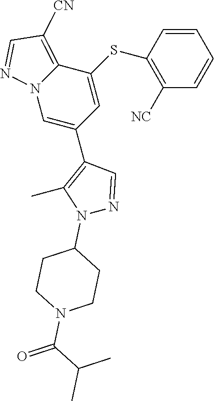 [SMILES: Cc1c(-c2cc(Sc3ccccc3C#N)c3c(C#N)cnn3c2)cnn1C1CCN(C(=O)C(C)C)CC1] | 4-((2-cyanophenyl)thio)- 6-(1-(1- isobutyrylpiperidin-4-yl)- 5-methyl-1H-pyrazol-4- yl)pyrazolo[1,5- a]pyridine-3-carbonitrile | Example 36 | 510.1 | |
| 423 | 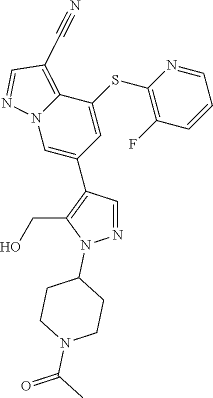 [SMILES: CC(=O)N1CCC(n2ncc(-c3cc(Sc4ncccc4F)c4c(C#N)cnn4c3)c2CO)CC1] | 6-(1-(1-acetylpiperidin-4- yl)-5-(hydroxymethyl)- 1H-pyrazol-4-yl)-4-((3- fluoropyridin-2- yl)thio)pyrazolo[1,5- a]pyridine-3-carbonitrile | Example 39 | 492.3 | 259 |
| 424 | 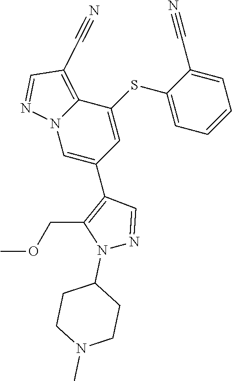 [SMILES: COCc1c(-c2cc(Sc3ccccc3C#N)c3c(C#N)cnn3c2)cnn1C1CCN(C)CC1] | 4-((2-cyanophenyl)thio)- 6-(5-(methoxymethyl)-1- (1-methylpiperidin-4-yl)- 1H-pyrazol-4- yl)pyrazolo[1,5- a]pyridine-3-carbonitrile | Example 53 | 484.3 | 46 |
| 425 | 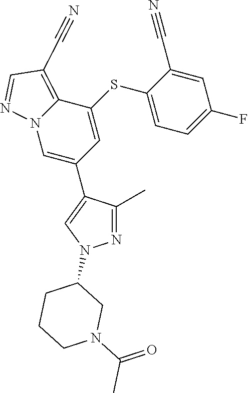 [SMILES: CC(=O)N1CCC[C@H](n2cc(-c3cc(Sc4ccc(F)cc4C#N)c4c(C#N)cnn4c3)c(C)n2)C1] | (S)-6-(1-(1- acetylpiperidin-3-yl)-3- methyl-1H-pyrazol-4-yl)- 4-((2-cyano-4- fluorophenyl)thio)pyrazolo [1,5-a]pyridine-3- carbonitrile | Example 30 | 500.3 | 6 |
| 426 | 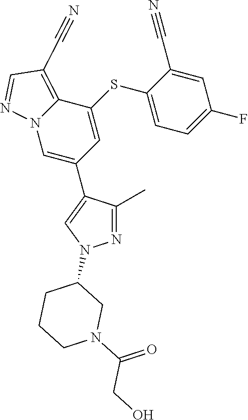 [SMILES: Cc1nn([C@H]2CCCN(C(=O)CO)C2)cc1-c1cc(Sc2ccc(F)cc2C#N)c2c(C#N)cnn2c1] | (S)-4-((2-cyano-4- fluorophenyl)thio)-6-(1- (1-(2- hydroxyacetyl)piperidin- 3-yl)-3-methyl-1H- pyrazol-4-yl)pyrazolo[1,5- a]pyridine-3-carbonitrile | Example 30 | 516.2 | 4 |
| 427 | 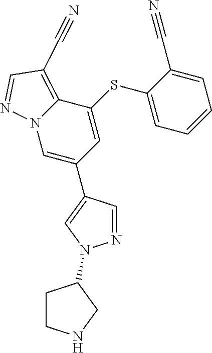 | (S)-4-((2- cyanophenyl)thio)-6-(1- (pyrrolidin-3-yl)-1H- pyrazol-4-yl)pyrazolo[1,5- a]pyridine-3-carbonitrile | Example 39 | 412.1 | 0.4 |
| 428 | 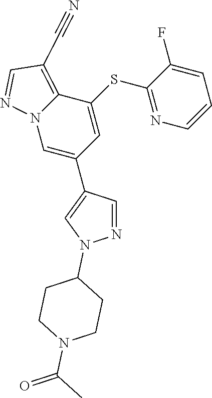 [SMILES: CC(=O)N1CCC(n2cc(-c3cc(Sc4ncccc4F)c4c(C#N)cnn4c3)cn2)CC1] | 6-(1-(1-acetylpiperidin-4- yl)-1H-pyrazol-4-yl)-4- ((3-fluoropyridin-2- yl)thio)pyrazolo[1,5- a]pyridine-3-carbonitrile | Example 39 | 462.1 | 1 |
| 429 | 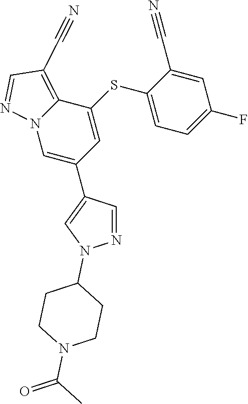 [SMILES: CC(=O)N1CCC(n2cc(-c3cc(Sc4ccc(F)cc4C#N)c4c(C#N)cnn4c3)cn2)CC1] | 6-(1-(1-acetylpiperidin-4- yl)-1H-pyrazol-4-yl)-4- ((2-cyano-4- fluorophenyl)thio)pyrazolo [1,5-a]pyridine-3- carbonitrile | Example 39 | 486.2 | 1 |
| 430 | 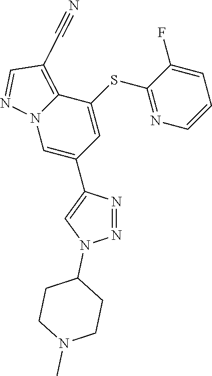 [SMILES: CN1CCC(n2cc(-c3cc(Sc4ncccc4F)c4c(C#N)cnn4c3)nn2)CC1] | 4-((3-fluoropyridin-2- yl)thio)-6-(1-(1- methylpiperidin-4-yl)-1H- 1,2,3-triazol-4- yl)pyrazolo[1,5- a]pyridine-3-carbonitrile | Example 59 | 435.1 | 10 |
| 431 | 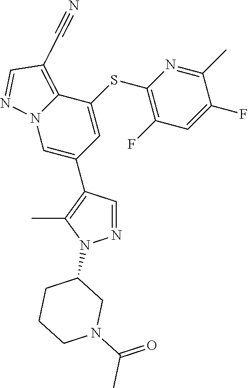 [SMILES: CC(=O)N1CCC[C@H](n2ncc(-c3cc(Sc4nc(C)c(F)cc4F)c4c(C#N)cnn4c3)c2C)C1] | (S)-6-(1-(1- acetylpiperidin-3-yl)-5- methyl-1H-pyrazol-4-yl)- 4-((3,5-difluoro-6- methylpyridin-2- yl)thio)pyrazolo[1,5- a]pyridine-3-carbonitrile | Example 39 | 508.2 | 2 |
| 432 | 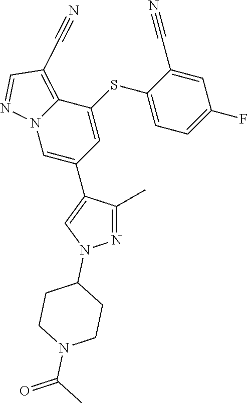 [SMILES: CC(=O)N1CCC(n2cc(-c3cc(Sc4ccc(F)cc4C#N)c4c(C#N)cnn4c3)c(C)n2)CC1] | 6-(1-(1-acetylpiperidin-4- yl)-3-methyl-1H-pyrazol- 4-yl)-4-((2-cyano-4- fluorophenyl)thio)pyrazolo [1,5-a]pyridine-3- carbonitrile | Example 39 | 500.2 | 3 |
| 433 | 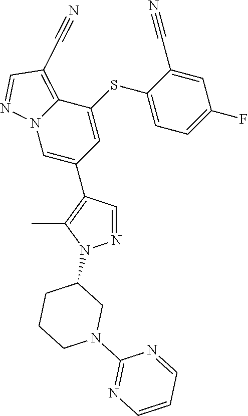 [SMILES: Cc1c(-c2cc(Sc3ccc(F)cc3C#N)c3c(C#N)cnn3c2)cnn1[C@H]1CCCN(c2ncccn2)C1] | (S)-4-((2-cyano-4- fluorophenyl)thio)-6-(5- methyl-1-(1-(pyrimidin-2- yl)piperidin-3-yl)-1H- pyrazol-4-yl)pyrazolo[1,5- a]pyridine-3-carbonitrile | Example 35 | 536.2 | 5 |
| 434 | 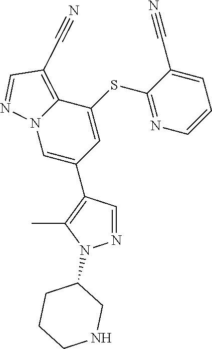 [SMILES: Cc1c(-c2cc(Sc3ncccc3C#N)c3c(C#N)cnn3c2)cnn1[C@H]1CCCNC1] | (S)-4-((3-cyanopyridin-2- yl)thio)-6-(5-methyl-1- (piperidin-3-yl)-1H- pyrazol-4-yl)pyrazolo[1,5- a]pyridine-3-carbonitrile | Example 53 | 441.2 | 1 |
| 435 | 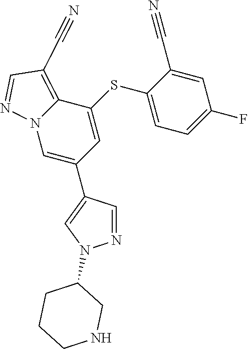 [SMILES: N#Cc1cc(F)ccc1Sc1cc(-c2cnn([C@H]3CCCNC3)c2)cn2ncc(C#N)c12] | (S)-4-((2-cyano-4- fluorophenyl)thio)-6-(1- (piperidin-3-yl)-1H- pyrazol-4-yl)pyrazolo[1,5- a]pyridine-3-carbonitrile | Example 53 | 444.2 | 1 |
| 436 | 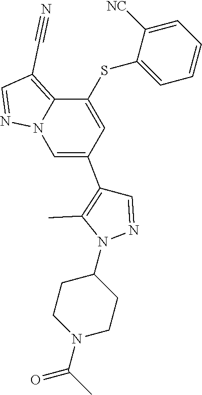 [SMILES: CC(=O)N1CCC(n2ncc(-c3cc(Sc4ccccc4C#N)c4c(C#N)cnn4c3)c2C)CC1] | 6-(1-(1-acetylpiperidin-4- yl)-5-methyl-1H-pyrazol- 4-yl)-4-((2- cyanophenyl)thio)pyrazolo [1,5-a]pyridine-3- carbonitrile | Example 39 | 482.2 | 1 |
| 437 | 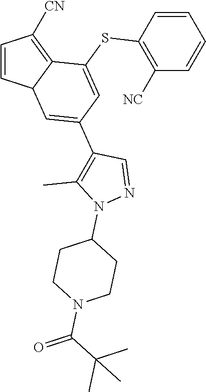 [SMILES: Cc1c(C2=CC3C=CC(C#N)=C3C(Sc3ccccc3C#N)=C2)cnn1C1CCN(C(=O)C(C)(C)C)CC1] | 4-((2-cyanophenyl)thio)- 6-(5-methyl-1-(1- pivaloylpiperidin-4-yl)- 1H-pyrazol-4- yl)pyrazolo[1,5- a]pyridine-3-carbonitrile | Example 36 | 524.2 | 1 |
| 438 | 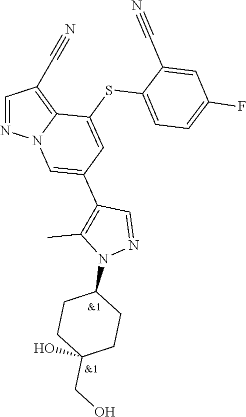 [SMILES: Cc1c(-c2cc(Sc3ccc(F)cc3C#N)c3c(C#N)cnn3c2)cnn1[C@H]1CC[C@@](O)(CO)CC1] | 4-((2-cyano-4- fluorophenyl)thio)-6-(1- ((1r,4r)-4-hydroxy-4- (hydroxymethyl)cyclohex- yl)-5-methyl-1H-pyrazol- 4-yl)pyrazolo[1,5- a]pyridine-3-carbonitrile | Example 61 | 503.2 | 1 |
| 439 | 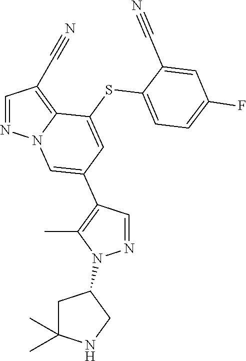 [SMILES: Cc1c(-c2cc(Sc3ccc(F)cc3C#N)c3c(C#N)cnn3c2)cnn1[C@@H]1CNC(C)(C)C1] | (S)-4-((2-cyano-4- fluorophenyl)thio)-6-(1- (5,5-dimethylpyrrolidin-3- yl)-5-methyl-1H-pyrazol- 4-yl)pyrazolo[1,5- a]pyridine-3-carbonitrile | Example 53 | 472.1 | 1 |
| 440 | 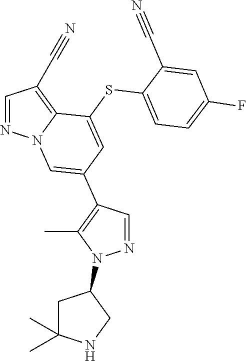 [SMILES: Cc1c(-c2cc(Sc3ccc(F)cc3C#N)c3c(C#N)cnn3c2)cnn1[C@H]1CNC(C)(C)C1] | (R)-4-((2-cyano-4- fluorophenyl)thio)-6-(1- (5,5-dimethylpyrrolidin-3- yl)-5-methyl-1H-pyrazol- 4-yl)pyrazolo[1,5- a]pyridine-3-carbonitrile | Example 53 | 472.1 | 2 |
| 441 | 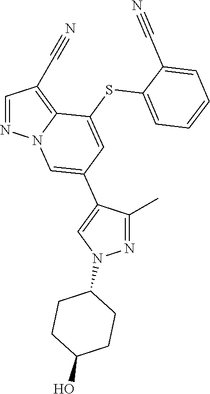 [SMILES: Cc1nn([C@H]2CC[C@H](O)CC2)cc1-c1cc(Sc2ccccc2C#N)c2c(C#N)cnn2c1] | 4-((2-cyanophenyl)thio)- 6-(1-((1r,4r)-4- hydroxycyclohexyl)-3- methyl-1H-pyrazol-4- yl)pyrazolo[1,5- a]pyridine-3-carbonitrile | Example 53 | 455.2 | 3 |
| 442 | 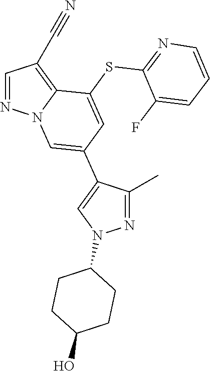 [SMILES: Cc1nn([C@H]2CC[C@H](O)CC2)cc1-c1cc(Sc2ncccc2F)c2c(C#N)cnn2c1] | 4-((3-fluoropyridin-2- yl)thio)-6-(1-((1r,4r)-4- hydroxycyclohexyl)-3- methyl-1H-pyrazol-4- yl)pyrazolo[1,5- a]pyridine-3-carbonitrile | Example 53 | 449.1 | 8 |
| 443 | 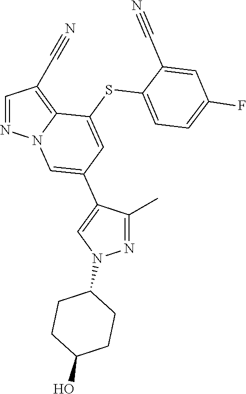 [SMILES: Cc1nn([C@H]2CC[C@H](O)CC2)cc1-c1cc(Sc2ccc(F)cc2C#N)c2c(C#N)cnn2c1] | 4-((2-cyano-4- fluorophenyl)thio)-6-(1- ((1r,4r)-4- hydroxycyclohexyl)-3- methyl-1H-pyrazol-4- yl)pyrazolo[1,5- a]pyridine-3-carbonitrile | Example 53 | 473.1 | 4 |
| 444 | 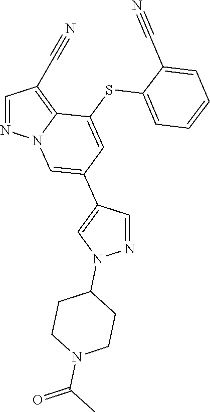 [SMILES: CC(=O)N1CCC(n2cc(-c3cc(Sc4ccccc4C#N)c4c(C#N)cnn4c3)cn2)CC1] | 6-(1-(1-acetylpiperidin-4- yl)-1H-pyrazol-4-yl)-4- ((2- cyanophenyl)thio)pyrazolo [1,5-a]pyridine-3- carbonitrile | Example 39 | 468.1 | 0.3 |
| 445 | 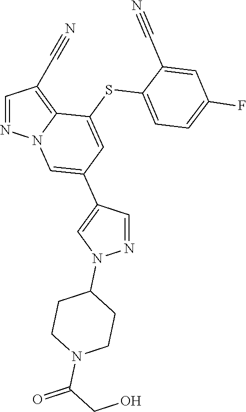 [SMILES: N#Cc1cc(F)ccc1Sc1cc(-c2cnn(C3CCN(C(=O)CO)CC3)c2)cn2ncc(C#N)c12] | 4-((2-cyano-4- fluorophenyl)thio)-6-(1- (1-(2- hydroxyacetyl)piperidin- 4-yl)-1H-pyrazol-4- yl)pyrazolo[1,5- a]pyridine-3-carbonitrile | Example 36 | 502.1 | 1 |
| 446 | 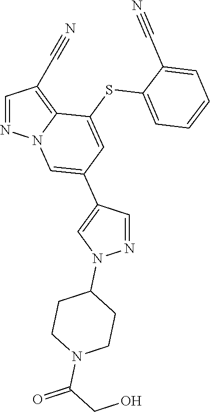 [SMILES: N#Cc1ccccc1Sc1cc(-c2cnn(C3CCN(C(=O)CO)CC3)c2)cn2ncc(C#N)c12] | 4-((2-cyanophenyl)thio)- 6-(1-(1-(2- hydroxyacetyl)piperidin- 4-yl)-1H-pyrazol-4- yl)pyrazolo[1,5- a]pyridine-3-carbonitrile | Example 36 | 484.4 | 0.5 |
| 447 | 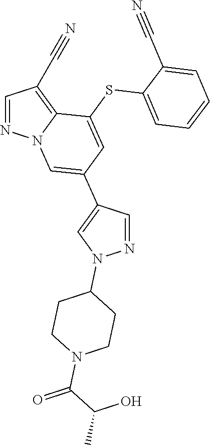 [SMILES: C[C@@H](O)C(=O)N1CCC(n2cc(-c3cc(Sc4ccccc4C#N)c4c(C#N)cnn4c3)cn2)CC1] | (R)-4-((2- cyanophenyl)thio)-6-(1- (1-(2- hydroxypropanoyl)piperidin- 4-yl)-1H-pyrazol-4- yl)pyrazolo∥,5- a]pyridine-3-carbonitrile | Example 36 | 498.1 | 0.4 |
| 448 | 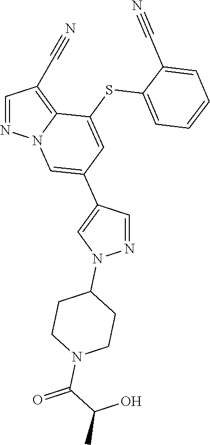 [SMILES: C[C@H](O)C(=O)N1CCC(n2cc(-c3cc(Sc4ccccc4C#N)c4c(C#N)cnn4c3)cn2)CC1] | (S)-4-((2- cyanophenyl)thio)-6-(1- (1-(2- hydroxypropanoyl)piperidin- 4-yl)-1H-pyrazol-4- yl)pyrazolo[1,5- a]pyridine-3-carbonitrile | Example 36 | 468.1 | 0.3 |
| 449 | 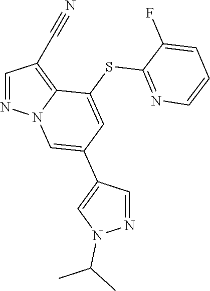 [SMILES: CC(C)n1cc(-c2cc(Sc3ncccc3F)c3c(C#N)cnn3c2)cn1] | 4-((3-fluoropyridin-2- yl)thio)-6-(1-isopropyl- 1H-pyrazol-4- yl)pyrazolo[1,5- a]pyridine-3-carbonitrile | Example 53 | 379.1 | 2 |
| 450 | 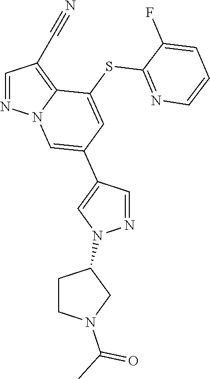 [SMILES: CC(=O)N1CC[C@H](n2cc(-c3cc(Sc4ncccc4F)c4c(C#N)cnn4c3)cn2)C1] | (S)-6-(1-(1- acetylpyrrolidin-3-yl)-1H- pyrazol-4-yl)-4-((3- fluoropyridin-2- yl)thio)pyrazolo[1,5- a]pyridine-3-carbonitrile | Example 39 | 448.3 | 1 |
| 451 | 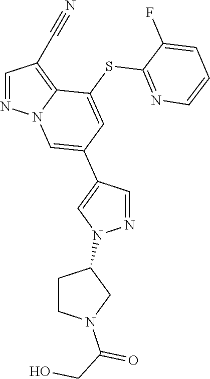 [SMILES: N#Cc1cnn2cc(-c3cnn([C@H]4CCN(C(=O)CO)C4)c3)cc(Sc3ncccc3F)c12] | (S)-4-((3-fluoropyridin-2- yl)thio)-6-(1-(1-(2- hydroxyacetyl)pyrrolidin- 3-yl)-1H-pyrazol-4- yl)pyrazolo[1,5- a]pyridine-3-carbonitrile | Example 36 | 464.0 | 1 |
| 452 | 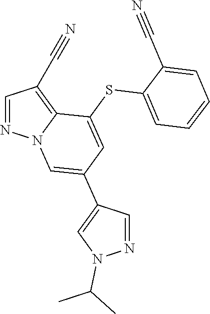 [SMILES: CC(C)n1cc(-c2cc(Sc3ccccc3C#N)c3c(C#N)cnn3c2)cn1] | 4-((2-cyanophenyl)thio)- 6-(1-isopropyl-1H- pyrazol-4-yl)pyrazolo[1,5- a]pyridine-3-carbonitrile | Example 53 | 385.1 | 0.6 |
| 453 | 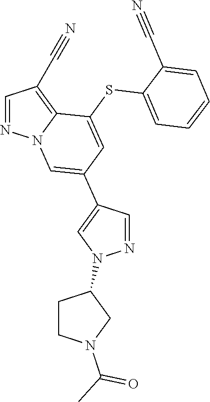 [SMILES: CC(=O)N1CC[C@H](n2cc(-c3cc(Sc4ccccc4C#N)c4c(C#N)cnn4c3)cn2)C1] | (S)-6-(1-(1- acetylpyrrolidin-3-yl)-1H- pyrazol-4-yl)-4-((2- cyanophenyl)thio)pyrazolo [1,5-a]pyridine-3- carbonitrile | Example 39 | 454.1 | 0.4 |
| 454 | 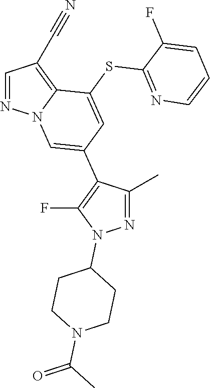 [SMILES: CC(=O)N1CCC(n2nc(C)c(-c3cc(Sc4ncccc4F)c4c(C#N)cnn4c3)c2F)CC1] | 6-(1-(1-acetylpiperidin-4- yl)-5-fluoro-3-methyl-1H- pyrazol-4-yl)-4-((3- fluoropyridin-2- yl)thio)pyrazolo[1,5- a]pyridine-3-carbonitrile | Example 39 | 494.2 | 12 |
| 455 | 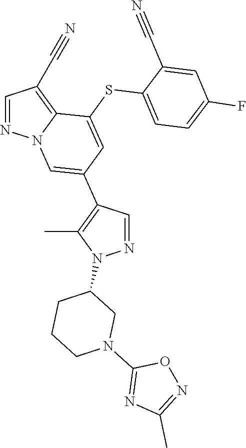 [SMILES: Cc1noc(N2CCC[C@H](n3ncc(-c4cc(Sc5ccc(F)cc5C#N)c5c(C#N)cnn5c4)c3C)C2)n1] | (S)-4-((2-cyano-4- fluorophenyl)thio)-6-(5- methyl-1-(1-(3-methyl- 1,2,4-oxadiazol-5- yl)piperidin-3-yl)-1H- pyrazol-4-yl)pyazolo[1,5- a]pyridine-3-carbonitrile | Example 62 | 540.3 | 2 |
| 456 | 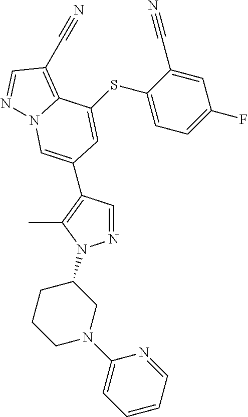 [SMILES: Cc1c(-c2cc(Sc3ccc(F)cc3C#N)c3c(C#N)cnn3c2)cnn1[C@H]1CCCN(c2ccccn2)C1] | (S)-4-((2-cyano-4- fluorophenyl)thio)-6-(5- methyl-1-(1-(pyridin-2- yl)piperidin-3-yl)-1H- pyrazol-4-yl)pyrazolo[1,5- a]pyridine-3-carbonitrile | Example 35 | 535.2 | 8 |
| 457 | 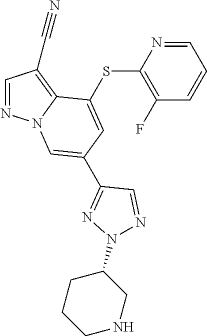 [SMILES: N#Cc1cnn2cc(-c3cnn([C@H]4CCCNC4)n3)cc(Sc3ncccc3F)c12] | S)-4-((3-fluoropyridin-2- yl)thio)-6-(2-(piperidin-3- yl)-2H-1,2,3-triazol-4- yl)pyrazolo[1,5- a]pyridine-3-carbonitrile | Example 63 | 421.3 | 13 |
| 458 | 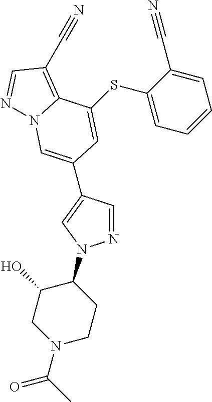 [SMILES: CC(=O)N1CC[C@H](n2cc(-c3cc(Sc4ccccc4C#N)c4c(C#N)cnn4c3)cn2)[C@@H](O)C1] | 6-(1-((3S,4S)-1-acetyl-3- hydroxypiperidin-4-yl)- 1H-pyrazol-4-yl)-4-((2- cyanophenyl)thio)pyrazolo [1,5-a]pyridine-3- carbonitrile | Example 39 | 484.0 | 1 |
| 459 | 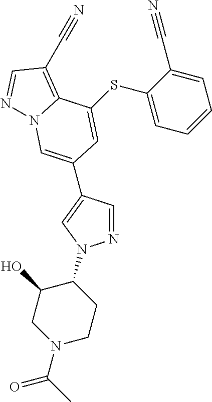 [SMILES: CC(=O)N1CC[C@@H](n2cc(-c3cc(Sc4ccccc4C#N)c4c(C#N)cnn4c3)cn2)[C@H](O)C1] | 6-(1-((3R,4R)-1-acetyl-3- hydroxypiperidin-4-yl)- 1H-pyrazol-4-yl)-4-((2- cyanophenyl)thio)pyrazolo [1,5-a]pyridine-3- carbonitrile | Example 39 | 484.0 | 0.4 |
| 460 | 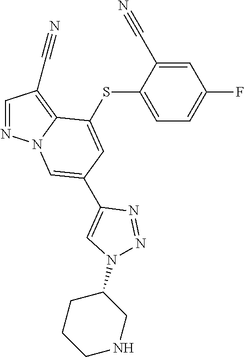 [SMILES: N#Cc1cc(F)ccc1Sc1cc(-c2cn([C@H]3CCCNC3)nn2)cn2ncc(C#N)c12] | (S)-4-((2-cyano-4- fluorophenyl)thio)-6-(1- (piperidin-3-yl)-1H-1,2,3- triazol-4-yl)pyrazolo[1,5- a]pyridine-3-carbonitrile | Example 59 | 445.3 | 6 |
| 461 | 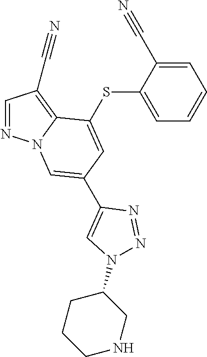 [SMILES: N#Cc1ccccc1Sc1cc(-c2cn([C@H]3CCCNC3)nn2)cn2ncc(C#N)c12] | (S)-4-((2- cyanophenyl)thio)-6-(1- (piperidin-3-yl)-1H-1,2,3- triazol-4-yl)pyrazolo[1,5- a]pyridine-3-carbonitrile | Example 59 | 427.2 | 4 |
| 462 | 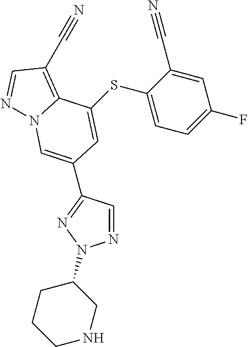 [SMILES: N#Cc1cc(F)ccc1Sc1cc(-c2cnn([C@H]3CCCNC3)n2)cn2ncc(C#N)c12] | (S)-4-((2-cyano-4- fluorophenyl)thio)-6-(2- (piperidin-3-yl)-2H-1,2,3- triazol-4-yl)pyrazolo[1,5- a]pyridine-3-carbonitrile | Example 63 | 445.4 | 19 |
| 463 | 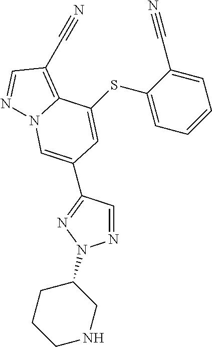 [SMILES: N#Cc1ccccc1Sc1cc(-c2cnn([C@H]3CCCNC3)n2)cn2ncc(C#N)c12] | (S)-4-((2- cyanophenyl)thio)-6-(2- (piperidin-3-yl)-2H-1,2,3- triazol-4-yl)pyrazolo[1,5- a]pyridine-3-carbonitrile | Example 63 | 427.1 | 6 |
| 464 | 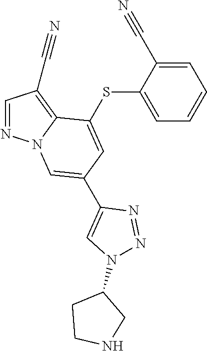 [SMILES: N#Cc1ccccc1Sc1cc(-c2cn([C@H]3CCNC3)nn2)cn2ncc(C#N)c12] | (S)-4-((2- cyanophenyl)thio)-6-(1- (pyrrolidin-3-yl)-1H- 1,2,3-triazol-4- yl)pyrazolo[1,5- a]pyridine-3-carbonitrile | Example 59 | 413.1 | 5 |
| 465 | 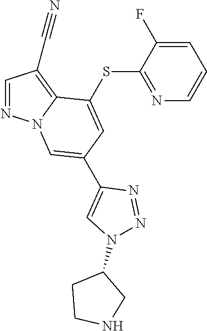 [SMILES: N#Cc1cnn2cc(-c3cn([C@H]4CCNC4)nn3)cc(Sc3ncccc3F)c12] | (S)-4-((3-fluoropyridin-2- yl)thio)-6-(1-(pyrrolidin- 3-yl)-1H-1,2,3-triazol-4- yl)pyrazolo[1,5- a]pyridine-3-carbonitrile | Example 59 | 407.1 | 11 |
| 466 | 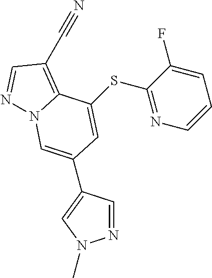 [SMILES: Cn1cc(-c2cc(Sc3ncccc3F)c3c(C#N)cnn3c2)cn1] | 4-((3-fluoropyridin-2- yl)thio)-6-(1-methyl-1H- pyrazol-4-yl)pyrazolo[1,5- a]pyridine-3-carbonitrile | Example 53 | 351.0 | 6 |
| 467 | 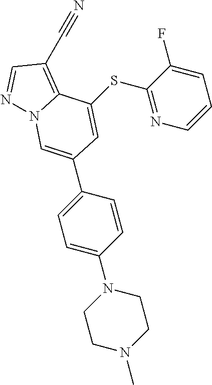 [SMILES: CN1CCN(c2ccc(-c3cc(Sc4ncccc4F)c4c(C#N)cnn4c3)cc2)CC1] | 4-((3-fluoropyridin-2- yl)thio)-6-(4-(4- methylpiperazin-1- yl)phenyl)pyrazolo[1,5- a]pyridine-3-carbonitrile | Example 53 | 445.2 | 0.4 |
| 468 | 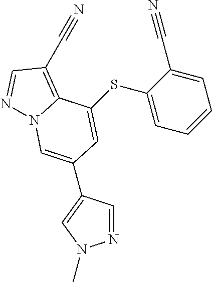 [SMILES: Cn1cc(-c2cc(Sc3ccccc3C#N)c3c(C#N)cnn3c2)cn1] | 4-((2-cyanophenyl)thio)- 6-(1-methyl-1H-pyrazol- 4-yl)pyrazolo[1,5- a]pyridine-3-carbonitrile | Example 53 | 357.1 | 1 |
| 469 | 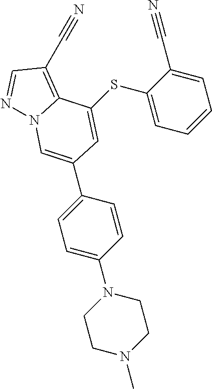 [SMILES: CN1CCN(c2ccc(-c3cc(Sc4ccccc4C#N)c4c(C#N)cnn4c3)cc2)CC1] | 4-((2-cyanophenyl)thio)- 6-(4-(4-methylpiperazin- 1-yl)phenyl)pyrazolo[1,5- a]pyridin-3-carbonitrile | Example 53 | 451.2 | 0.2 |
| 470 | 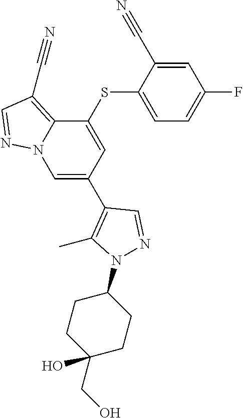 [SMILES: Cc1c(-c2cc(Sc3ccc(F)cc3C#N)c3c(C#N)cnn3c2)cnn1[C@H]1CC[C@](O)(CO)CC1] | 4-((2-cyano-4- fluorophenyl)thio)-6-(1- ((1s,4s)-4-hydroxy-4- (hydroxymethyl)cyclohex- yl)-5-methyl-1H-pyrazol- 4-yl)pyrazolo[1,5- a]pyridine-3-carbonitrile | Example 61 | 503.3 | 1 |
| 471 | 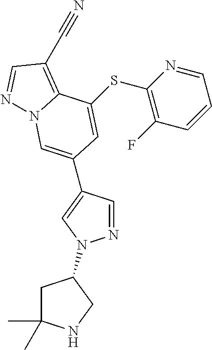 [SMILES: CC1(C)C[C@H](n2cc(-c3cc(Sc4ncccc4F)c4c(C#N)cnn4c3)cn2)CN1] | (S)-6-(1-(5,5- dimethylpyrrolidin-3-yl)- 1H-pyrazol-4-yl)-4-((3- fluoropyridin-2- yl)thio)pyrazolo[1,5- a]pyridine-3-carbonitrile | Example 53 | 434.2 | 1 |
| 472 | 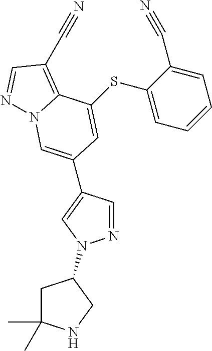 [SMILES: CC1(C)C[C@H](n2cc(-c3cc(Sc4ccccc4C#N)c4c(C#N)cnn4c3)cn2)CN1] | (S)-4-((2- cyanophenyl)thio)-6-(1- (5,5-dimethylpyrrolidin-3- yl)-1H-pyrazol-4- yl)pyrazolo[1,5- a]pyridine-3-carbonitrile | Example 53 | 440.2 | 0.3 |
| 473 | 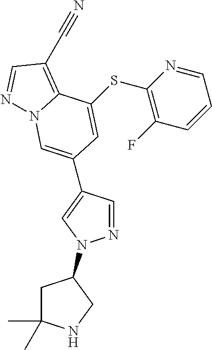 [SMILES: CC1(C)C[C@@H](n2cc(-c3cc(Sc4ncccc4F)c4c(C#N)cnn4c3)cn2)CN1] | (R)-6-(1-(5,5- dimethylpyrrolidin-3-yl)- 1H-pyrazol-4-yl)-4-((3- fluoropyridin-2- yl)thio)pyrazolo[1,5- a]pyridine-3-carbonitrile | Example 53 | 434.1 | 1 |
| 474 | 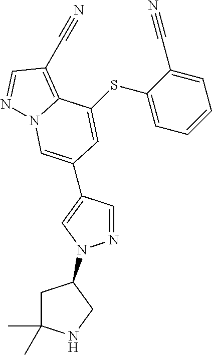 [SMILES: CC1(C)C[C@@H](n2cc(-c3cc(Sc4ccccc4C#N)c4c(C#N)cnn4c3)cn2)CN1] | (R)-4-((2- cyanophenyl)thio)-6-(1- (5,5-dimethylpyrrolidin-3- yl)-1H-pyrazol-4- yl)pyrazolo[1,5- a]pyridine-3-carbonitrile | Example 53 | 440.1 | 0.2 |
| 475 | 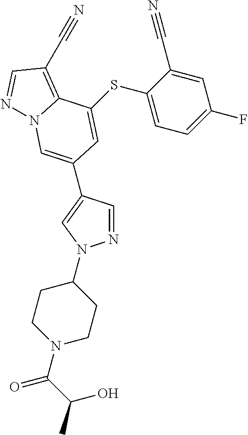 [SMILES: C[C@H](O)C(=O)N1CCC(n2cc(-c3cc(Sc4ccc(F)cc4C#N)c4c(C#N)cnn4c3)cn2)CC1] | (S)-4-((2-cyano-4- fluorophenyl)thio)-6-(1- (1-(2- hydroxypropanoyl)piperidin- 4-yl)-1H-pyrazol-4- yl)pyrazolo[1,5- a]pyridine-3-carbonitrile | Example 36 | 516.2 | 1 |
| 476 | 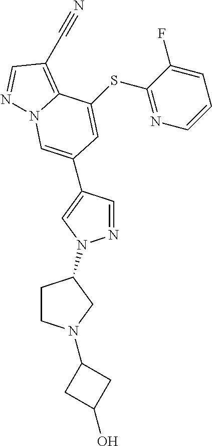 [SMILES: N#Cc1cnn2cc(-c3cnn([C@H]4CCN(C5CC(O)C5)C4)c3)cc(Sc3ncccc3F)c12] | (S)-4-((3-fluoropyridin-2- yl)thio)-6-(1-(1-(3- hydroxycyclobutyl)pyrroli- din-3-yl)-1H-pyrazol-4- yl)pyrazolo[1,5- a]pyridine-3-carbonitrile | Example 32 | 476.1 | 1 |
| 477 | 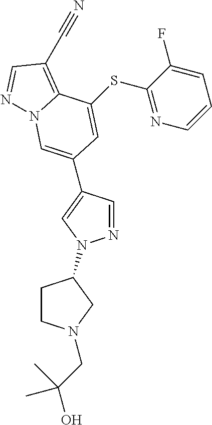 [SMILES: CC(C)(O)CN1CC[C@H](n2cc(-c3cc(Sc4ncccc4F)c4c(C#N)cnn4c3)cn2)C1] | (S)-4-((3-fluoropyridin-2- yl)thio)-6-(1-(1-(2- hydroxy-2- methylpropyl)pyrrolidin- 3-yl)-1H-pyrazol-4- y)pyrazolo[1,5- a]pyridine-3-carbonitrile | Example 35 | | 1 |
| 478 | 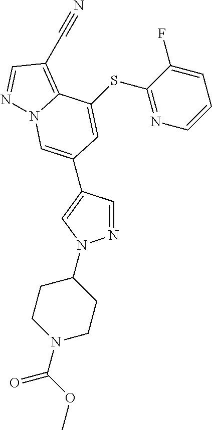 [SMILES: COC(=O)N1CCC(n2cc(-c3cc(Sc4ncccc4F)c4c(C#N)cnn4c3)cn2)CC1] | methyl 4-(4-(3-cyano-4- ((3-fluoropyridin-2- yl)thio)pyrazolo[1,5- a]pyridin-6-yl)-1H- pyrazol-1-yl)piperidine-1- carboxylate | Example 39 | 478.3 | 1 |
| 479 | 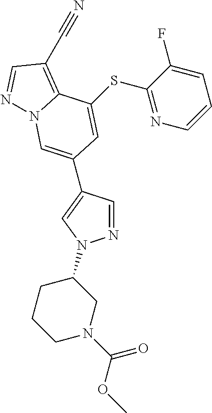 [SMILES: COC(=O)N1CCC[C@H](n2cc(-c3cc(Sc4ncccc4F)c4c(C#N)cnn4c3)cn2)C1] | methyl (S)-3-(4-(3-cyano- 4-((3-fluoropyridin-2- yl)thio)pyrazolo[1,5- a]pyridin-6-yl)-1H- pyrazol-1-yl)piperidine-1- carboxylate | Example 39 | 478.1 | 9 = 1 |
| 480 | 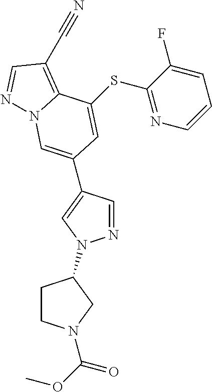 [SMILES: COC(=O)N1CC[C@H](n2cc(-c3cc(Sc4ncccc4F)c4c(C#N)cnn4c3)cn2)C1] | methyl (S)-3-(4-(3-cyano- 4-((3-fluoropyridin-2- yl)thio)pyrazolo[1,5- a]pyridin-6-yl)-1H- pyrazol-1-yl)pyrrolidine- 1-carboxylate | Example 39 | 478.1 | 1 |
| 481 | 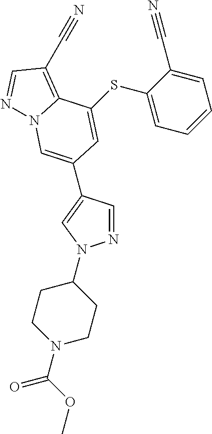 [SMILES: COC(=O)N1CCC(n2cc(-c3cc(Sc4ccccc4C#N)c4c(C#N)cnn4c3)cn2)CC1] | methyl 4-(4-(3-cyano-4- ((2- cyanophenyl)thio)pyrazolo [1,5-a]pyridin-6-yl)-1H- pyrazol-1-yl)piperidine-1- carboxylate | Example 39 | 484.4 | 04 |
| 482 | 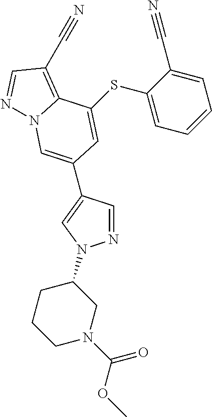 [SMILES: COC(=O)N1CCC[C@H](n2cc(-c3cc(Sc4ccccc4C#N)c4c(C#N)cnn4c3)cn2)C1] | methyl (S)-3-(4-(3-cyano- 4-((2- cyanophenyl)thio)pyrazolo [1,5-a]pyridin-6-yl)-1H- pyrazol-1-yl)piperidine-1- carboxylate | Exmaple 39 | 484.2 | 1 |
| 483 | 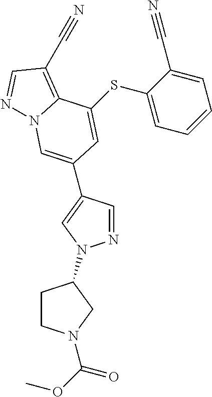 [SMILES: COC(=O)N1CC[C@H](n2cc(-c3cc(Sc4ccccc4C#N)c4c(C#N)cnn4c3)cn2)C1] | methyl (S)-3-(4-(3-cyano- 4-((2- cyanophenyl)thio)pyrazolo [1,5-a]pyridin-6-yl)-1H- pyrazol-1-yl)pyrrolidine- 1-carboxylate | Example 39 | 470.2 | 0.5 |
| 484 | 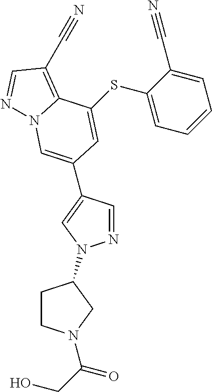 [SMILES: N#Cc1ccccc1Sc1cc(-c2cnn([C@H]3CCN(C(=O)CO)C3)c2)cn2ncc(C#N)c12] | (S)-4-((2- cyanophenyl)thio)-6-(1- (1-(2- hydroxyacetyl)pyrrolidin- 3-yl)-1H-pyrazol-4- yl)pyrazolo[1,5- a]pyridine-3-carbonitrile | Example 36 | 470.1 | 0.4 |
| 485 | 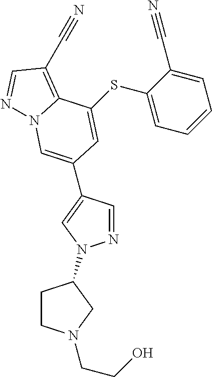 [SMILES: N#Cc1ccccc1Sc1cc(-c2cnn([C@H]3CCN(CCO)C3)c2)cn2ncc(C#N)c12] | (S)-4-((2- cyanophenyl)thio)-6-(1- (1-(2- hydroxyethyl)pyrrolidin- 3-yl)-1H-pyrazol-4- yl)pyrazolo[1,5- a]pyridine-3-carbonitrile | Example 35 | 456.1 | 0.4 |
| 486 | 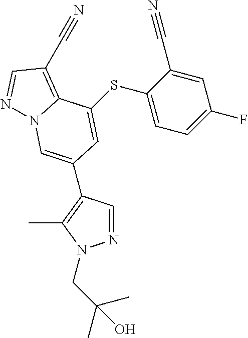 [SMILES: Cc1c(-c2cc(Sc3ccc(F)cc3C#N)c3c(C#N)cnn3c2)cnn1CC(C)(C)O] | 4-((2-cyano-4- fluorophenyl)thio)-6-(1- (2-hydroxy-2- methylpropyl)-5-methyl- 1H-pyrazol-4- yl)pyrazolo[1,5- a]pyridine-3-carbonitrile | Example 35 | 447.1 | 9 |
| 487 | 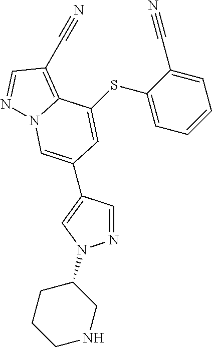 [SMILES: N#Cc1ccccc1Sc1cc(-c2cnn([C@H]3CCCNC3)c2)cn2ncc(C#N)c12] | (S)-4-((2- cyanophenyl)thio)-6-(1- (piperidin-3-yl)-1H- pyrazol-4-yl)pyrazolo[1,5- a]pyridine-3-carbonitrile | Example 53 | 426.3 | 0.4 |
| 488 | 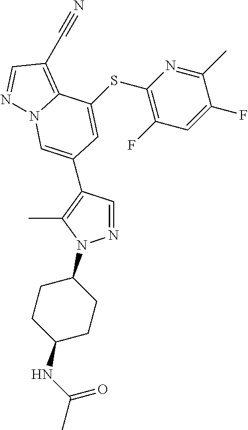 [SMILES: CC(=O)N[C@H]1CC[C@@H](n2ncc(-c3cc(Sc4nc(C)c(F)cc4F)c4c(C#N)cnn4c3)c2C)CC1] | N-((1s,4s)-4-(4-(3-cyano- 4-((3,5-difluoro-6- methylpyridin-2- yl)thio)pyrazolo[1,5- a]pyridin-6-yl)-5-methyl- 1H-pyrazol-1- yl)cyclohexyl)acetamide | Example 39 | 522.2 | 1 |
| 489 | 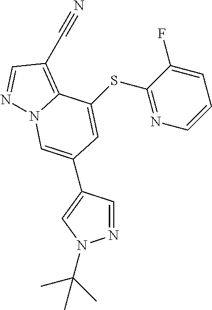 [SMILES: CC(C)(C)n1cc(-c2cc(Sc3ncccc3F)c3c(C#N)cnn3c2)cn1] | 6-(1-(tert-butyl)-1H- pyrazol-4-yl)-4-((3- fluoropyridin-2- y)thio)pyrazolo[1,5- a]pyridine-3-carbonitrile | Example 53 | 393.2 | 3 |
| 490 | 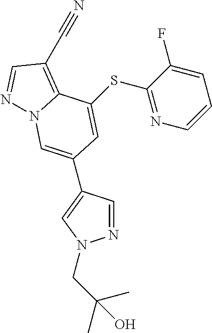 [SMILES: CC(C)(O)Cn1cc(-c2cc(Sc3ncccc3F)c3c(C#N)cnn3c2)cn1] | 4-((3-fluoropyridin-2- yl)thio)-6-(1-(2-hydroxy- 2-methylpropyl)-1H- pyrazol-4-yl)pyrazolo[1,5- a]pyridine-3-carbonitrile | Example 35 | 409.0 | 2 |
| 491 | 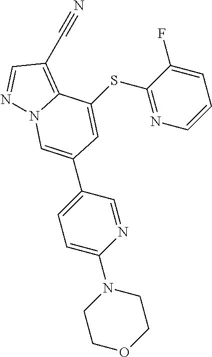 [SMILES: N#Cc1cnn2cc(-c3ccc(N4CCOCC4)nc3)cc(Sc3ncccc3F)c12] | 4-((3-fluoropyridin-2- yl)thio)-6-(6- morpholinopyridin-3- yl)pyrazolo[1,5- a]pyridine-3-carbonitrile | Example 53 | 432.1 | 1 |
| 492 | 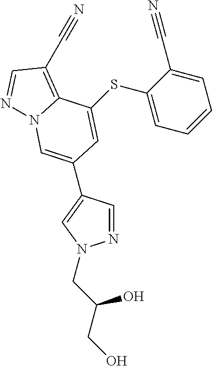 [SMILES: N#Cc1ccccc1Sc1cc(-c2cnn(C[C@@H](O)CO)c2)cn2ncc(C#N)c12] | (R)-4-((2- cyanophenyl)thio)-6-(1- (2,3-dihydroxypropyl)- 1H-pyrazol-4- yl)pyrazolo[1,5- a]pyridine-3-carbonitrile | Example 35 | 417.2 | 1 |
| 493 | 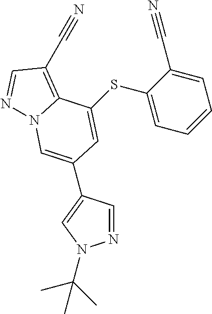 [SMILES: CC(C)(C)n1cc(-c2cc(Sc3ccccc3C#N)c3c(C#N)cnn3c2)cn1] | 6-(1-(tert-butyl)-1H- pyrazol-4-yl)-4-((2- cyanophenyl)thio)pyrazolo [1,5-a]pyridine-3- carbonitrile | Example 53 | 399.1 | 1 |
| 494 | 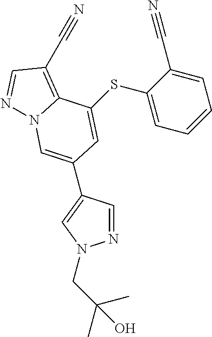 [SMILES: CC(C)(O)Cn1cc(-c2cc(Sc3ccccc3C#N)c3c(C#N)cnn3c2)cn1] | 4-((2-cyanophenyl)thio)- 6-(1-(2-hydroxy-2- methylpropyl)-1H- pyrazol-4-yl)pyrazolo[1,5- a]pyridine-3-carbonitrile | Example 39 | 415.2 | 1 |
| 495 | 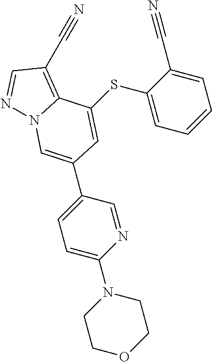 [SMILES: N#Cc1ccccc1Sc1cc(-c2ccc(N3CCOCC3)nc2)cn2ncc(C#N)c12] | 4-((2-cyanophenyl)thio)- 6-(6-morpholinopyridin-3- yl)pyrazolo[1,5- a]pyridine-3-carbonitrile | Example 53 | 439.2 | 1 |
| 496 | 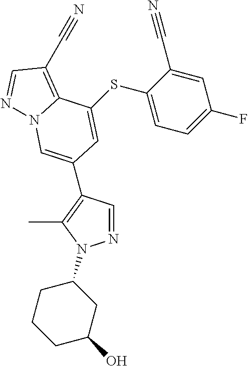 [SMILES: Cc1c(-c2cc(Sc3ccc(F)cc3C#N)c3c(C#N)cnn3c2)cnn1[C@H]1CCC[C@H](O)C1] | 4-((2-cyano-4- fluorophenyl)thio)-6-(1- ((1S,3S)-3- hydroxycyclohexyl)-5- methyl-1H-pyrazol-4- yl)pyrazolo[1,5- a]pyridine-3-carbonitrile | Example 53 | 473.4 | 3 |
| 497 | 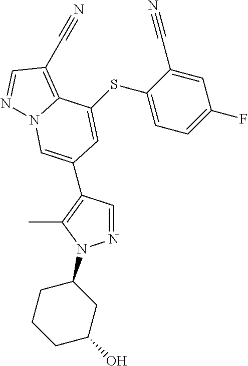 [SMILES: Cc1c(-c2cc(Sc3ccc(F)cc3C#N)c3c(C#N)cnn3c2)cnn1[C@@H]1CCC[C@@H](O)C1] | 4-((2-cyano-4- fluorophenyl)thio)-6-(1- ((1R,3R)-3- hydroxycyclohexyl)-5- methyl-1H-pyrazol-4- y)pyrazolo[1,5- a]pyridine-3-carbonitrile | Example 53 | 473.3 | 1 |
| 498 | 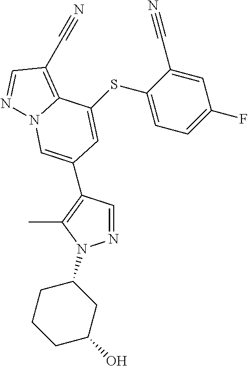 [SMILES: Cc1c(-c2cc(Sc3ccc(F)cc3C#N)c3c(C#N)cnn3c2)cnn1[C@H]1CCC[C@@H](O)C1] | 4-((2-cyano-4- fluorophenyl)thio)-6-(1- ((1S,3R)-3- hydroxycyclohexyl)-5- methyl-1H-pyrazol-4- yl)pyrazolo[1,5- a]pyridine-3-carbonitrile | Example 53 | 473.2 | 2 |
| 499 | 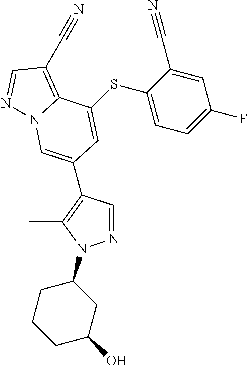 [SMILES: Cc1c(-c2cc(Sc3ccc(F)cc3C#N)c3c(C#N)cnn3c2)cnn1[C@@H]1CCC[C@H](O)C1] | 4-((2-cyano-4- fluorophenyl)thio)-6-(1- ((1R,3S)-3- hydroxycyclohexyl)-5- methyl-1H-pyrazol-4- yl)pyrazolo[1,5- a]pyridine-3-carbonitrile | Example 53 | 473.2 | 1 |
| 500 | 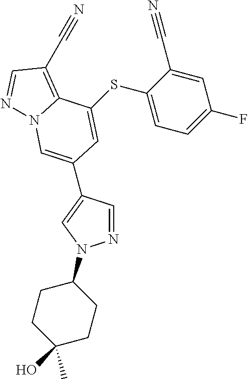 [SMILES: C[C@]1(O)CC[C@@H](n2cc(-c3cc(Sc4ccc(F)cc4C#N)c4c(C#N)cnn4c3)cn2)CC1] | 4-((2-cyano-4- fluorophenyl)thio)-6-(1- ((1s,4s)-4-hydroxy-4- methylcyclohexyl)-1H- pyrazol-4-yl)pyrazolo[1,5- a]pyridine-3-carbonitrile | Example 54 | 473.2 | 1 |
| 501 | 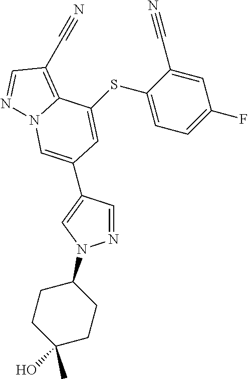 [SMILES: C[C@]1(O)CC[C@H](n2cc(-c3cc(Sc4ccc(F)cc4C#N)c4c(C#N)cnn4c3)cn2)CC1] | 4-((2-cyano-4- fluorophenyl)thio)-6-(1- ((1r,4r)-4-hydroxy-4- methylcyclohexyl)-1H- pyrazol-4-yl)pyrazolo[1,5- a]pyridine-3-carbonitrile | Example 54 | 473.1 | 1 |
| 502 | 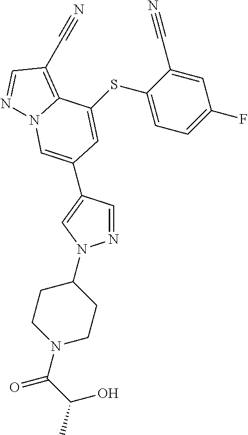 [SMILES: C[C@@H](O)C(=O)N1CCC(n2cc(-c3cc(Sc4ccc(F)cc4C#N)c4c(C#N)cnn4c3)cn2)CC1] | (R)-4-((2-cyano-4- fluorophenyl)thio)-6-(1- (1-(2- hydroxypropanoyl)piperidin- 4-yl)-1H-pyrazol-4- yl)pyrazolo[1,5- a]pyridine-3-carbonitrile | Example 36 | 516.2 | 1 |
| 503 | 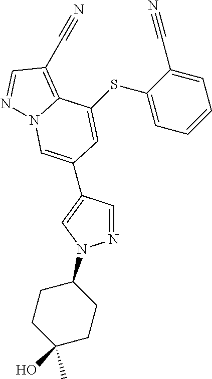 [SMILES: C[C@]1(O)CC[C@@H](n2cc(-c3cc(Sc4ccccc4C#N)c4c(C#N)cnn4c3)cn2)CC1] | 4-((2-cyanophenyl)thio)- 6-(1-((1s,4s)-4-hydroxy-4- methylcyclohexyl)-1H- pyrazol-4-yl)pyrazolo[1,5- a]pyridine-3-carbonitrile | Example 54 | 455.3 | 0.5 |
| 504 | 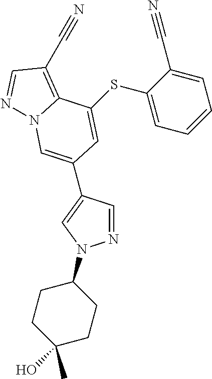 [SMILES: C[C@]1(O)CC[C@H](n2cc(-c3cc(Sc4ccccc4C#N)c4c(C#N)cnn4c3)cn2)CC1] | 4-((2-cyanophenyl)thio)- 6-(1-((1r,4r)-4-hydroxy-4- methylcyclohexyl)-1H- pyrazol-4-yl)pyrazolo[1,5- a]pyridine-3-carbonitrile | Example 54 | 455.3 | 0.4 |
| 505 | 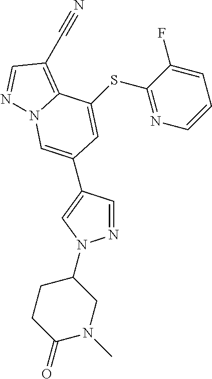 [SMILES: CN1CC(n2cc(-c3cc(Sc4ncccc4F)c4c(C#N)cnn4c3)cn2)CCC1=O] | 4-((3-fluoropyridin-2- yl)thio)-6-(1-(1-methyl-6- oxopiperidin-3-yl)-1H- pyrazol-4-yl)pyrazolo[1,5- a]pyridine-3-carbonitrile | Example 53 | 448.3 | 3 |
| 506 | 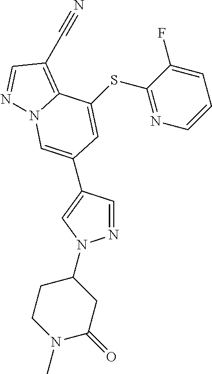 [SMILES: CN1CCC(n2cc(-c3cc(Sc4ncccc4F)c4c(C#N)cnn4c3)cn2)CC1=O] | 4-((3-fluoropyridin-2- yl)thio)-6-(1-(1-methyl-2- oxopiperidin-4-yl)-1H- pyrazol-4-yl)pyrazolo[1,5- a]pyridine-3-carbonitrile | Example 53 | 448.2 | 1 |
| 507 | 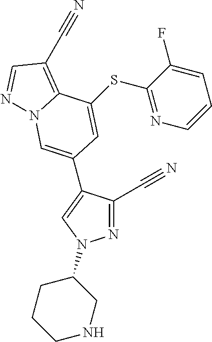 [SMILES: N#Cc1nn([C@H]2CCCNC2)cc1-c1cc(Sc2ncccc2F)c2c(C#N)cnn2c1] | (S)-6-(3-cyano-1- (piperidin-3-yl)-1H- pyrazol-4-yl)-4-((3- fluoropyridin-2- y)thio)pyrazolo[1,5- a]pyridine-3-carbonitrile | Example 53 | 445.2 | 30 |
| 508 | 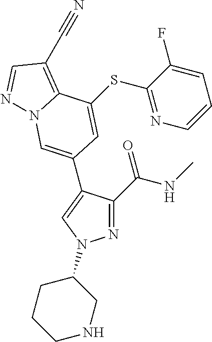 [SMILES: CNC(=O)c1nn([C@H]2CCCNC2)cc1-c1cc(Sc2ncccc2F)c2c(C#N)cnn2c1] | (S)-4-(3-cyano-4-((3- fluoropyridin-2- yl)thio)pyrazolo[1,5- a]pyridin-6-yl)-N-methyl- 1-(piperidin-3-yl)-1H- pyrazole-3-carboxamide | Example 53 | 477.1 | 48 |
| 509 | 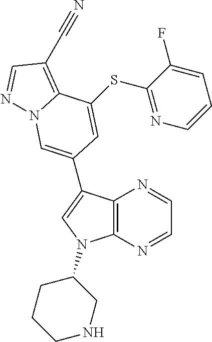 [SMILES: N#Cc1cnn2cc(-c3cn([C@H]4CCCNC4)c4nccnc34)cc(Sc3ncccc3F)c12] | (S)-4-((3-fluoropyridin-2- yl)thio)-6-(5-(piperidin-3- yl)-5H-pyrrolo[2,3- b]pyrazin-7- yl)pyrazolo[1,5- a]pyridine-3-carbonitrile | Example 53 | 471.0 | 22 |
| 510 | 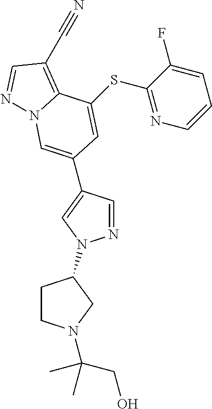 [SMILES: CC(C)(CO)N1CC[C@H](n2cc(-c3cc(Sc4ncccc4F)c4c(C#N)cnn4c3)cn2)C1] | (S)-4-((3-fluoropyridin-2- yl)thio)-6-(1-(1-(1- hydroxy-2-methylpropan- 2-yl)pyrrolidin-3-yl)-1H- pyrazol-4-yl)pyrazolo[1,5- a]pyridine-3-carbonitrile | Example 35 | 478.0 | 1 |
| 511 | 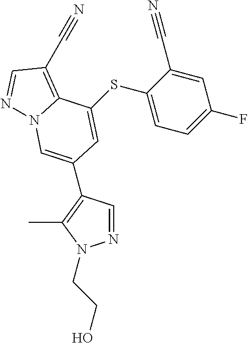 [SMILES: Cc1c(-c2cc(Sc3ccc(F)cc3C#N)c3c(C#N)cnn3c2)cnn1CCO] | 4-((2-cyano-4- fluorophenyl)thio)-6-(1- (2-hydroxyethyl)-5- methyl-1H-pyrazol-4- yl)pyrazolo[1,5- a]pyridine-3-carbonitrile | Example 35 | 419.1 | 8 |
| 512 | 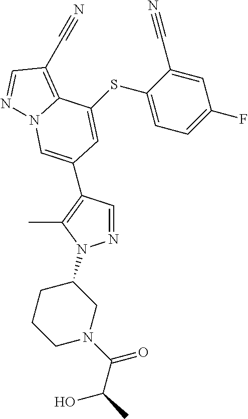 [SMILES: Cc1c(-c2cc(Sc3ccc(F)cc3C#N)c3c(C#N)cnn3c2)cnn1[C@H]1CCCN(C(=O)[C@@H](C)O)C1] | 4-((2-cyano-4- fluorophenyl)thio)-6-(1- ((S)-1-((R)-2- hydroxypropanoyl)piperidin- 3-yl)-5-methyl-1H- pyrazol-4-yl)pyrazolo[1,5- a]pyridine-3-carbonitrile | Example 30 | 530.4 | 1 |
| 513 | 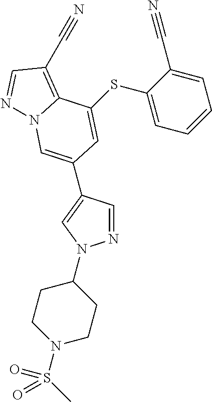 [SMILES: CS(=O)(=O)N1CCC(n2cc(-c3cc(Sc4ccccc4C#N)c4c(C#N)cnn4c3)cn2)CC1] | 4-((2-cyanophenyl)thio)- 6-(1-(1- (methylsulfonyl)piperidin- 4-yl)-1H-pyrazol-4- yl)pyrazolo[1,5- a]pyridine-3-carbonitrile | Example 50 | 504.1 | 1 |
| 514 | 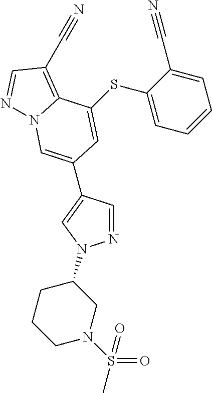 [SMILES: CS(=O)(=O)N1CCC[C@H](n2cc(-c3cc(Sc4ccccc4C#N)c4c(C#N)cnn4c3)cn2)C1] | (S)-4-((2- cyanophenyl)thio)-6-(1- (1- (methylsulfonyl)piperidin- 3-yl)-1H-pyrazol-4- yl)pyrazolo[1,5- a]pyridine-3-carbonitrile | Example 50 | 504.3 | 1 |
| 515 | 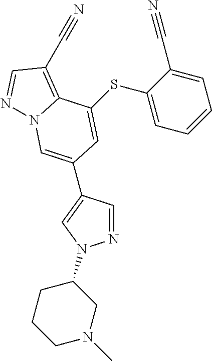 [SMILES: CN1CCC[C@H](n2cc(-c3cc(Sc4ccccc4C#N)c4c(C#N)cnn4c3)cn2)C1] | (S)-4-((2- cyanophenyl)thio)-6-(1- (1-methylpiepridin-3-yl)- 1H-pyrazol-4- yl)pyrazolo[1,5- a]pyridine-3-carbonitrile | Example 53 | 440.2 | 1 |
| 516 | 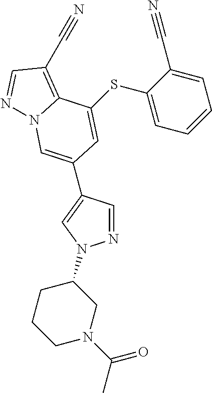 [SMILES: CC(=O)N1CCC[C@H](n2cc(-c3cc(Sc4ccccc4C#N)c4c(C#N)cnn4c3)cn2)C1] | (S)-6-(1-(1- acetylpiperidin-3-yl)-1H- pyrazol-4-yl)-4-((2- cyanophenyl)thio)pyrazolo [1,5-a]pyridine-3- carbonitrile | Example 39 | 468.1 | 0.4 |
| 517 | 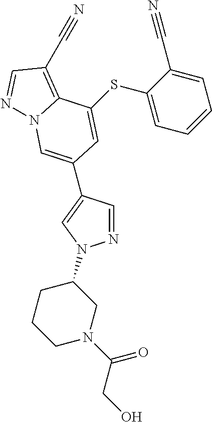 [SMILES: N#Cc1ccccc1Sc1cc(-c2cnn([C@H]3CCCN(C(=O)CO)C3)c2)cn2ncc(C#N)c12] | (S)-4-((2- cyanophenyl)thio)-6-(1- (1-(2- hydroxyacetyl)piperidin- 3-yl)-1H-pyrazol-4- yl)pyrazolo[1,5- a]pyridine-3-carbonitrile | Example 30 | 484.3 | 0.4 |
| 518 | 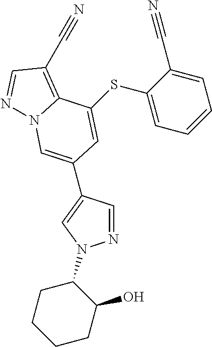 [SMILES: N#Cc1ccccc1Sc1cc(-c2cnn([C@H]3CCCC[C@@H]3O)c2)cn2ncc(C#N)c12] | 4-((2-cyanophenyl)thio)- 6-(1-((1S,2S)-2- hydroxycyclohexyl)-1H- pyrazol-4-yl)pyrazolo[1,5- a]pyridine-3-carbonitrile | Example 53 | 441.1 | 0.4 |
| 519 | 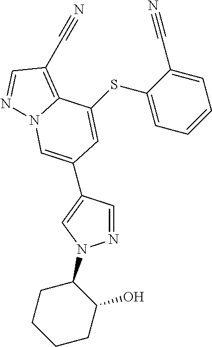 [SMILES: N#Cc1ccccc1Sc1cc(-c2cnn([C@@H]3CCCC[C@H]3O)c2)cn2ncc(C#N)c12] | 4-((2-cyanophenyl)thio)- 6-(1-((1R,2R)-2- hydroxycyclohexyl)-1H- pyrazol-4-yl)pyrazolo[1,5- a]pyridine-3-carbonitirle | Example 53 | 441.1 | 1 |
| 520 | 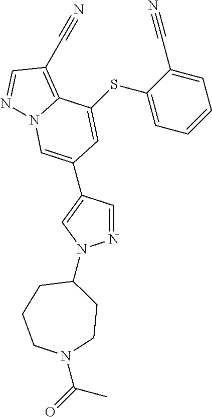 [SMILES: CC(=O)N1CCCC(n2cc(-c3cc(Sc4ccccc4C#N)c4c(C#N)cnn4c3)cn2)CC1] | 6-(1-(1-acetylazepan-4- yl)-1H-pyrazol-4-yl)-4- ((2- cyanophenyl)thio)pyrazolo [1,5-a]pyridine-3- carbonitrile | Example 39 | 482.2 | 0.4 |
| 521 | 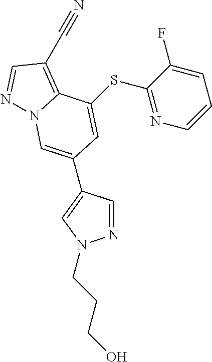 [SMILES: N#Cc1cnn2cc(-c3cnn(CCCO)c3)cc(Sc3ncccc3F)c12] | 4-((3-fluoropyridin-2- yl)thio)-6-(1-(3- hydroxypropyl)-1H- pyrazol-4-yl)pyrazolo[1,5- a]pyridine-3-carbonitrile | Example 35 | 395.1 | 2 |
| 522 | 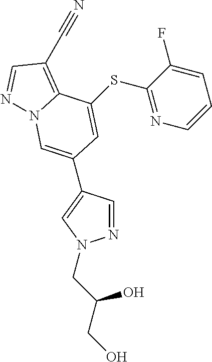 [SMILES: N#Cc1cnn2cc(-c3cnn(C[C@@H](O)CO)c3)cc(Sc3ncccc3F)c12] | (R)-6-(1-(2,3- dihydroxypropyl)-1H- pyrazol-4-yl)-4-((3- fluoropyridin-2- yl)thio)pyrazolo[1,5- a]pyridine-3-carbonitrile | Example 35 | 411.1 | 2 |
| 523 | 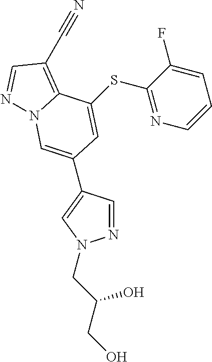 [SMILES: N#Cc1cnn2cc(-c3cnn(C[C@H](O)CO)c3)cc(Sc3ncccc3F)c12] | (S)-6-(1-(2,3- dihydroxypropyl)-1H- pyrazol-4-yl)-4-((3- fluoropyridin-2- yl)thio)pyrazolo[1,5- a]pyridine-3-carbonitrile | Example 35 | 411.0 | 2 |
| 524 | 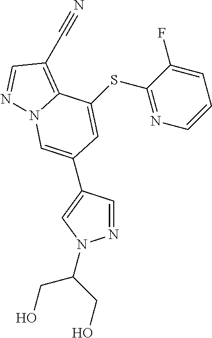 [SMILES: N#Cc1cnn2cc(-c3cnn(C(CO)CO)c3)cc(Sc3ncccc3F)c12] | 6-(1-(1,3- dihydroxypropan-2-yl)- 1H-pyrazol-4-yl)-4-((3- fluoropyridin-2- yl)thio)pyrazolo[1,5- a]pyridine-3-carbonitrile | Example 35 | 411.2 | 3 |
| 525 | 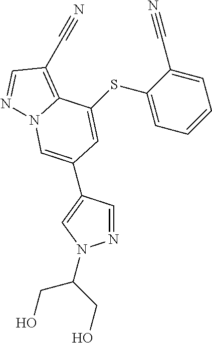 [SMILES: N#Cc1ccccc1Sc1cc(-c2cnn(C(CO)CO)c2)cn2ncc(C#N)c12] | 4-((2-cyanophenyl)thio)- 6-(1-(1,3- dihydroxypropan-2-yl)- 1H-pyrazol-4- yl)pyrazolo[1,5- a]pyridine-3-carbonitrile | Example 35 | 417.0 | 1 |
| 526 | 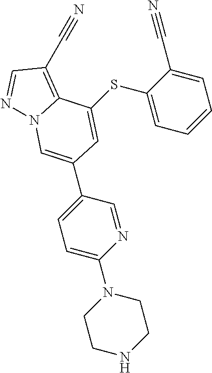 [SMILES: N#Cc1ccccc1Sc1cc(-c2ccc(N3CCNCC3)nc2)cn2ncc(C#N)c12] | 4-((2-cyanophenyl)thio)- 6-(6-(piperazin-1- yl)pyridin-3- yl)pyrazolo[1,5- a]pyridine-3-carbonitrile | Example 53 | 438.1 | 1 |
| 527 | 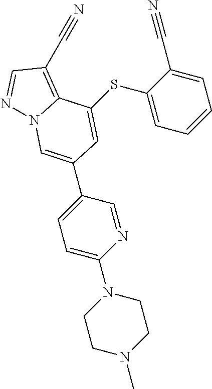 [SMILES: CN1CCN(c2ccc(-c3cc(Sc4ccccc4C#N)c4c(C#N)cnn4c3)cn2)CC1] | 4-((2-cyanophenyl)thio)- 6-(6-(4-methylpiperazin- 1-yl)pyridin-3- yl)pyrazolo[1,5- a]pyridine-3-carbonitrile | Example 53 | 452.3 | 0.4 |
| 528 | 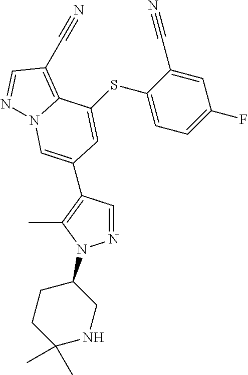 [SMILES: Cc1c(-c2cc(Sc3ccc(F)cc3C#N)c3c(C#N)cnn3c2)cnn1[C@@H]1CCC(C)(C)NC1] | (S)-4-((2-cyano-4- fluorophenyl)thio)-6-(1- (6,6-dimethylpiperidin-3- yl)-5-methyl-1H-pyrazol- 4-yl)pyrazolo[1,5- a]pyridine-3-carbonitrile | Example 64 | 486.3 | 2.5 |
| 529 | 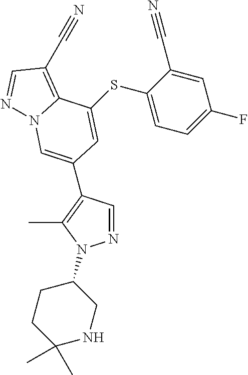 [SMILES: Cc1c(-c2cc(Sc3ccc(F)cc3C#N)c3c(C#N)cnn3c2)cnn1[C@H]1CCC(C)(C)NC1] | methyl 4-(4-(3-cyano-4- ((3-fluoropyridin-2- yl)thio)pyrazolo[1,5- a]pyridin-6-yl)-1H- pyrazol-1-yl)pyiperidine-1- carboxylate | Example 64 | 486.2 | 1 |
| 530 | 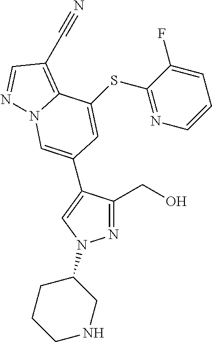 [SMILES: N#Cc1cnn2cc(-c3cn([C@H]4CCCNC4)nc3CO)cc(Sc3ncccc3F)c12] | (S)-4-((3-fluoropyridin-2- yl)thio)-6-(3- (hydroxymethyl)-1- (piperidin-3-yl)-1H- pyrazol-4-yl)pyrazolo[1,5- a]pyridine-3-carbonitrile | Example 65 | 450.3 | 69 |
| 531 | 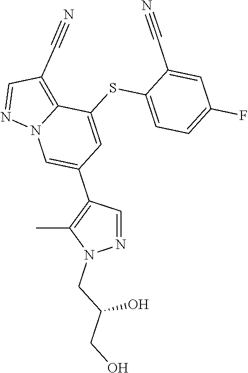 [SMILES: Cc1c(-c2cc(Sc3ccc(F)cc3C#N)c3c(C#N)cnn3c2)cnn1C[C@H](O)CO] | (S)-4-((2-cyano-4- fluorophenyl)thio)-6-(1- (2,3-dihydroxypropyl)-5- methyl-1H-pyrazol-4- yl)pyrazolo[1,5- a]pyridine-3-carbonitrile | Example 35 | 449.2 | 11 |
| 532 | 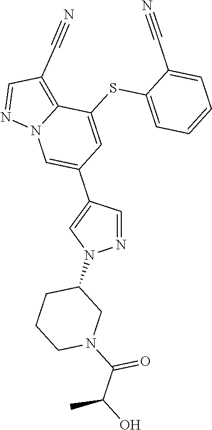 [SMILES: C[C@H](O)C(=O)N1CCC[C@H](n2cc(-c3cc(Sc4ccccc4C#N)c4c(C#N)cnn4c3)cn2)C1] | 4-((2-cyanophenyl)thio)- 6-(1-((S)-1-((S)-2- hydroxypropanoyl)piperidin- 3-yl)-1H-pyrazol-4- yl)pyrazolo[1,5- a]pyridine-3-carbonitrile | Example 30 | 498.2 | 0.5 |
| 533 | 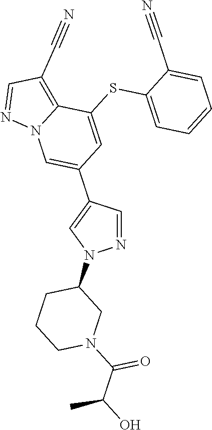 [SMILES: C[C@H](O)C(=O)N1CCC[C@@H](n2cc(-c3cc(Sc4ccccc4C#N)c4c(C#N)cnn4c3)cn2)C1] | 4-((2-cyanophenyl)thio)- 6-(1-((R)-1-((S)-2- hydroxypropanoyl)piperidin- 3-yl)-1H-pyrazol-4- yl)pyrazolo[1,5- a]pyridine-3-carbonitrile | Example 30 | 498.2 | 1 |
| 534 | 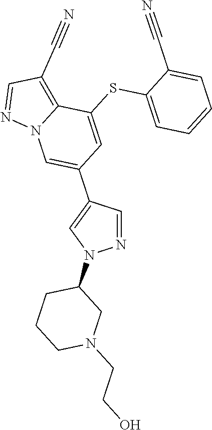 [SMILES: N#Cc1ccccc1Sc1cc(-c2cnn([C@@H]3CCCN(CCO)C3)c2)cn2ncc(C#N)c12] | (R)-4-((2- cyanophenyl)thio)-6-(1- (1-(2- hydroxyethyl)piperidin-3- yl)-1H-pyrazol-4- yl)pyrazolo[1,5- a]pyridine-3-carbonitrile | Example 35 | 470.2 | 0.5 |
| 535 | 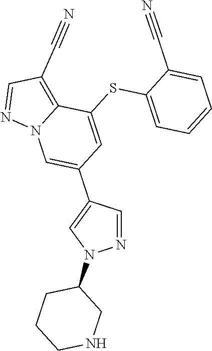 [SMILES: N#Cc1ccccc1Sc1cc(-c2cnn([C@@H]3CCCNC3)c2)cn2ncc(C#N)c12] | (R)-4-((2- cyanophenyl)thio)-6-(1- (piperidin-3-yl)-1H- pyrazol-4-yl)pyrazolo[1,5- a]pyridine-3-carbonitrile | Example 53 | 426.2 | 0.4 |
| 536 | 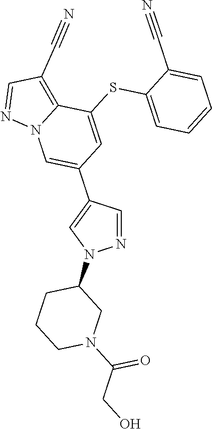 [SMILES: N#Cc1ccccc1Sc1cc(-c2cnn([C@@H]3CCCN(C(=O)CO)C3)c2)cn2ncc(C#N)c12] | (R)-4-((2- cyanophenyl)thio)-6-(1- (1-(2- hydroxyacetyl)piperidin- 3-yl)-1H-pyrazol-4- yl)pyrazolo[1,5- a]pyridine-3-carbonitrile | Example 30 | 484.3 | 1 |
| 537 | 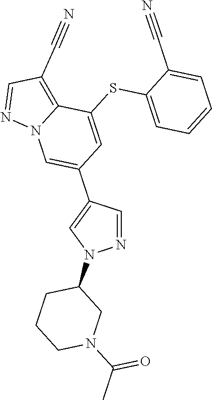 [SMILES: CC(=O)N1CCC[C@@H](n2cc(-c3cc(Sc4ccccc4C#N)c4c(C#N)cnn4c3)cn2)C1] | (R)-6-(1-(1- acetylpiperidin-3-yl)-1H- pyrazol-4-yl)-4-((2- cyanophenyl)thio)pyrazolo [1,5-a]pyridine-3- carbonitrile | Example 39 | 468.3 | 1 |
| 538 | 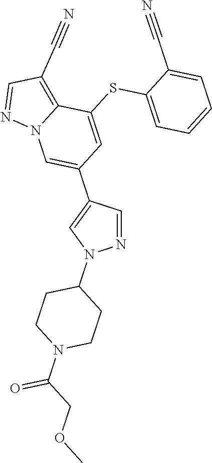 [SMILES: COCC(=O)N1CCC(n2cc(-c3cc(Sc4ccccc4C#N)c4c(C#N)cnn4c3)cn2)CC1] | 4-((2-cyanophenyl)thio)- 6-(1-(1-(2- methoxyacetyl)piperidin- 4-yl)-1H-pyrazol-4- yl)pyrazolo[1,5- a]pyridine-3-carbonitrile | Example 36 | 498.2 | 0.3 |
| 539 | 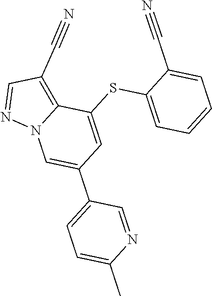 [SMILES: Cc1ccc(-c2cc(Sc3ccccc3C#N)c3c(C#N)cnn3c2)cn1] | 2-(4-(3-cyano-4-((2- cyanophenyl)thio)pyrazolo [1,5-a]pyridin-6-yl)-1H- pyrazol-1-yl)-N- methylacetamide | Example 53 | 368.1 | 9 |
| 540 | 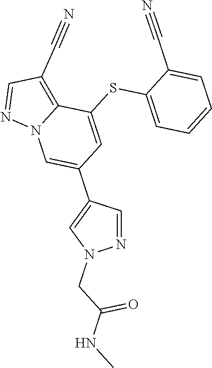 [SMILES: CNC(=O)Cn1cc(-c2cc(Sc3ccccc3C#N)c3c(C#N)cnn3c2)cn1] | 6-(1-(tert-butyl)-1H- pyrazol-4-yl)-4-((3- fluoropyridin-2- yl)thio)pyrazolo[1,5- a]pyridine-3-carbonitrile | Example 35 | 414.1 | 1 |
| 541 | 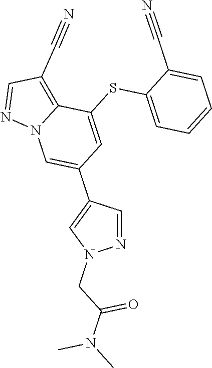 [SMILES: CN(C)C(=O)Cn1cc(-c2cc(Sc3ccccc3C#N)c3c(C#N)cnn3c2)cn1] | 2-(4-(3-cyano-4-((2- cyanophenyl)thio)pyrazolo [1,5-a]pyridin-6-yl)-1H- pyrazol-1-yl)-N,N- dimethylacetamide | Example 35 | 428.3 | 1 |
| 542 | 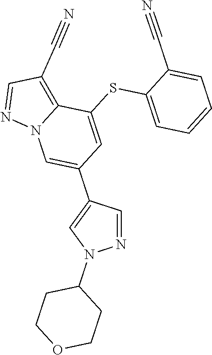 [SMILES: N#Cc1ccccc1Sc1cc(-c2cnn(C3CCOCC3)c2)cn2ncc(C#N)c12] | 4-((2-cyanophenyl)thio)- 6-(1-(tetrahydro-2H- pyran-4-yl)-1H-pyrazol-4- yl)pyrazolo[1,5- a]pyridine-3-carbonitrile | Example 35 | 427.0 | 0.4 |
| 543 | 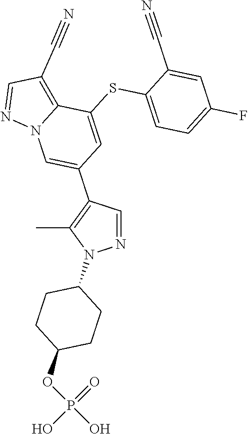 [SMILES: Cc1c(-c2cc(Sc3ccc(F)cc3C#N)c3c(C#N)cnn3c2)cnn1[C@H]1CC[C@H](OP(=O)(O)O)CC1] | (1r,4r)-4-(4-(3-cyano-4- ((2-cyano-4- fluorophenyl)thio)pyrazolo [1,5-a]pyridin-6-yl)-5- methyl-1H-pyrazol-1- yl)cyclohexyl dihydrogen phosphate | | 533.1 | 2 |
| 544 | 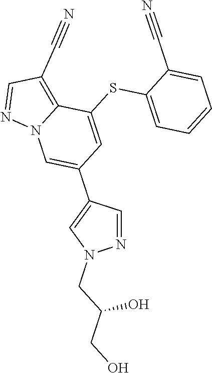 [SMILES: N#Cc1ccccc1Sc1cc(-c2cnn(C[C@H](O)CO)c2)cn2ncc(C#N)c12] | (S)-4-((2- cyanophenyl)thio)-6-(1- (2,3-dihydroxypropyl)- 1H-pyrazol-4- yl)pyrazolo[1,5- a]pyridine-3-carbonitrile | Example 35 | 417.0 | 1 |
| 545 | 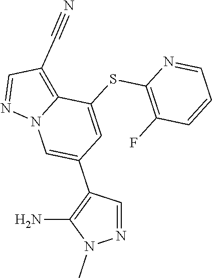 [SMILES: Cn1ncc(-c2cc(Sc3ncccc3F)c3c(C#N)cnn3c2)c1N] | 6-(5-amino-1-methyl-1H- pyrazol-4-yl)-4-((3- fluoropyridin-2- yl)thio)pyrazolo[1,5- a]pyridine-3-carbonitrile | | 366.2 | 3 |
| 546 | 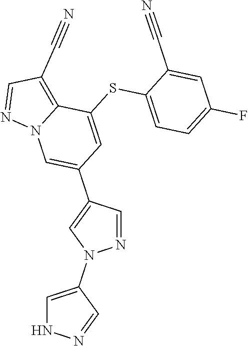 [SMILES: N#Cc1cc(F)ccc1Sc1cc(-c2cnn(-c3cn[nH]c3)c2)cn2ncc(C#N)c12] | 6-(1′H-[1,4′-bipyrazol]-4- yl)-4-((2-cyano-4- fluorophenyl)thio)pyrazolo [1,5-a]pyridine-3- carbonitrile | Example 53 | 427.1 | 6 |
| 547 | 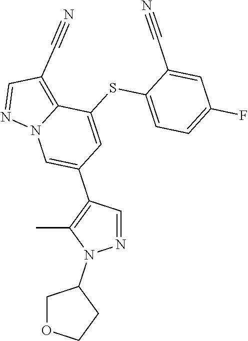 [SMILES: Cc1c(-c2cc(Sc3ccc(F)cc3C#N)c3c(C#N)cnn3c2)cnn1C1CCOC1] | 4-((2-cyano-4- fluorophenyl)thio)-6-(5- methyl-1- (tetrahydrofuran-3-yl)-1H- pyrazol-4-yl)pyrazolo[1,5- a]pyridine-3-carbonitrile | Example 35 | 445.2 | 3 |
| 548 | 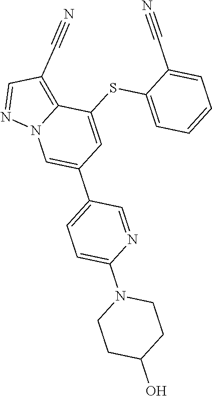 [SMILES: N#Cc1ccccc1Sc1cc(-c2ccc(N3CCC(O)CC3)nc2)cn2ncc(C#N)c12] | 4-((2-cyanophenyl)thio)- 6-(6-(4-hydroxypiperidin- 1-yl)pyridin-3- yl)pyrazolo[1,5- a]pyridine-3-carbonitrile | Example 53 | 453.2 | 0.4 |
| 549 | 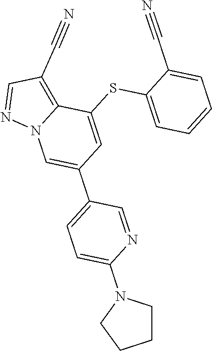 [SMILES: N#Cc1ccccc1Sc1cc(-c2ccc(N3CCCC3)nc2)cn2ncc(C#N)c12] | 4-((2-cyanophenyl)thio)- 6-(6-(pyrrolidin-1- yl)pyridin-3- yl)pyrazolo[1,5- a]pyridine-3-carbonitrile | Example 53 | 423.0 | 1 |
| 550 | 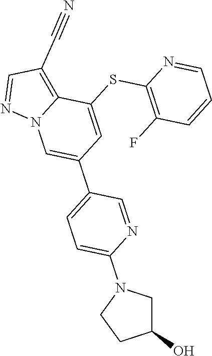 [SMILES: N#Cc1cnn2cc(-c3ccc(N4CC[C@H](O)C4)nc3)cc(Sc3ncccc3F)c12] | (S)-4-((3-fluoropyridin-2- yl)thio)-6-(6-(3- hydroxypryrrolidin-1- yl)pyridin-3- yl)pyrazolo[1,5- a]pyridine-3-carbonitrile | Example 53 | 433.4 | 1.5 |
| 551 | 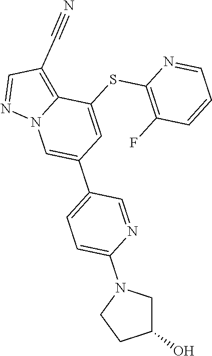 [SMILES: N#Cc1cnn2cc(-c3ccc(N4CC[C@@H](O)C4)nc3)cc(Sc3ncccc3F)c12] | (R)-4-((3-fluoropyridin-2- yl)thio)-6-(6-(3- hydroxypyrrolidin-1- yl)pyridin-3- yl)pyrazolo[1,5- a]pyridine-3-carbonitrile | Example 53 | 433.2 | 2 |
| 552 | 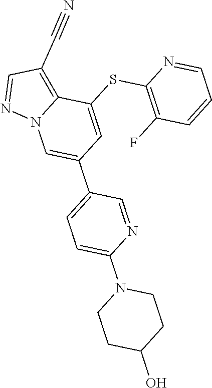 [SMILES: N#Cc1cnn2cc(-c3ccc(N4CCC(O)CC4)nc3)cc(Sc3ncccc3F)c12] | 4-((3-fluoropyridin-2- yl)thio)-6-(6-(4- hydroxypiperidin-1- yl)pyridin-3- yl)pyrazolo[1,5- a]pyridine-3-carbonitrile | Example 53 | 447.2 | 1 |
| 553 | 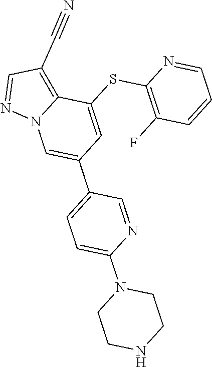 [SMILES: N#Cc1cnn2cc(-c3ccc(N4CCNCC4)nc3)cc(Sc3ncccc3F)c12] | 4-((3-fluoropyridin-2- yl)thio)-6-(6-(piperazin-1- yl)pyridin-3- yl)pyrazolo[1,5- a]pyridine-3-carbonitrile | Example 53 | 432.2 | 1 |
| 554 | 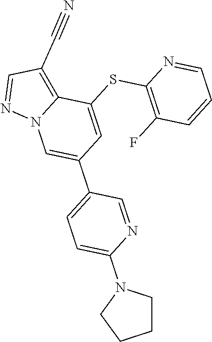 [SMILES: N#Cc1cnn2cc(-c3ccc(N4CCCC4)nc3)cc(Sc3ncccc3F)c12] | 4-((3-fluoropyridin-2- yl)thio)-6-(6-(pyrrolidin- 1-yl)pyridin-3- yl)pyrazolo[1,5- a]pyridine-3-carbonitrile | Example 53 | 417.1 | 2 |
| 555 | 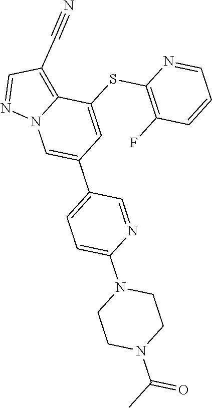 [SMILES: CC(=O)N1CCN(c2ccc(-c3cc(Sc4ncccc4F)c4c(C#N)cnn4c3)cn2)CC1] | 6-(6-(4-acetylpiperazin-1- yl)pyridin-3-yl)-4-((3- fluoropyridin-2- yl)thio)pyrazolo[1,5- a]pyridine-3-carbontrile | Example 53 | 474.1 | 1 |
| 556 | 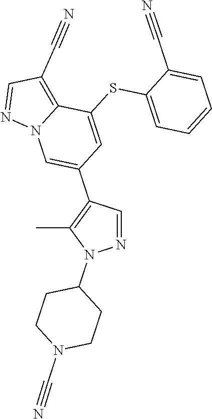 [SMILES: Cc1c(-c2cc(Sc3ccccc3C#N)c3c(C#N)cnn3c2)cnn1C1CCN(C#N)CC1] | 4-((2-cyanophenyl)thio)- 6-(1-(1-cyanopiperidin-4- yl)-5-methyl-1H-pyrazol- 4-yl)pyrazolo[1,5- a]pyridine-3-carbonitrile | Example 68 | 465.2 | 1 |
| 557 | 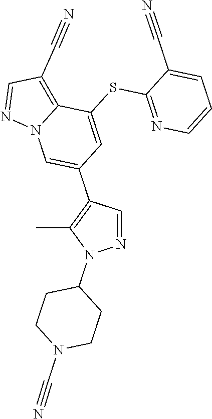 [SMILES: Cc1c(-c2cc(Sc3ncccc3C#N)c3c(C#N)cnn3c2)cnn1C1CCN(C#N)CC1] | 6-(1-(1-cyanopiperidin-4- yl)-5-methyl-1H-pyrazol- 4-yl)-4-((3-fluoropyridin- 2-yl)thio)pyrazolo∥,5- a]pyridine-3-carbonitrile | Example 68 | 459.0 | 2 |
| 558 | 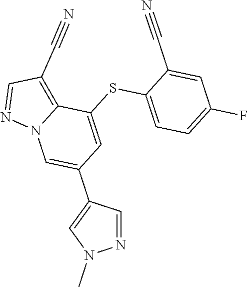 [SMILES: Cn1cc(-c2cc(Sc3ccc(F)cc3C#N)c3c(C#N)cnn3c2)cn1] | 4-((2-cyano-4- fluorophenyl)thio)-6-(1- methyl-1H-pyrazol-4- yl)pyrazolo[1,5- a]pyridine-3-carbonitrile | Example 53 | 375.1 | 2 |
| 559 | 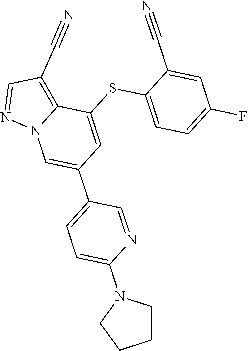 [SMILES: N#Cc1cc(F)ccc1Sc1cc(-c2ccc(N3CCCC3)nc2)cn2ncc(C#N)c12] | 4-((2-cyano-4- fluorophenyl)thio)-6-(6- (pyrrolidin-1-yl)pyridin-3- yl)pyrazolo[1,5- a]pyridine-3-carbonitrile | Example 53 | 441.0 | 1 |
| 560 | 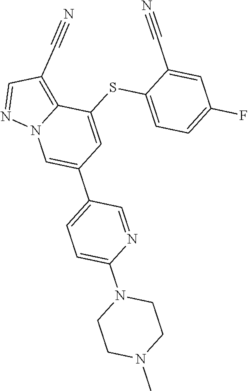 [SMILES: CN1CCN(c2ccc(-c3cc(Sc4ccc(F)cc4C#N)c4c(C#N)cnn4c3)cn2)CC1] | 4-((2-cyano-4- fluorophenyl)thio)-6-(6- (4-methylpiperazin-1- yl)pyridin-3- yl)pyrazolo[1,5- a]pyridine-3-carbonitrile | Example 53 | 470.2 | 1 |
| 561 | 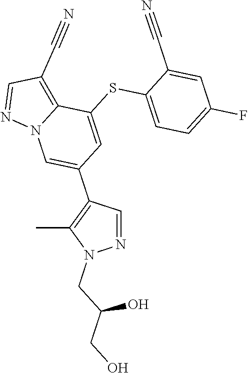 [SMILES: Cc1c(-c2cc(Sc3ccc(F)cc3C#N)c3c(C#N)cnn3c2)cnn1C[C@@H](O)CO] | (R)-4-((2-cyano-4- fluorophenyl)thio)-6-(1- (2,3-dihydroxypropyl)-5- methyl-1H-pyrazol-4- yl)pyrazolo[1,5- a]pyridine-3-carbonitrile | Example 35 | 449.3 | 7 |
| 562 | 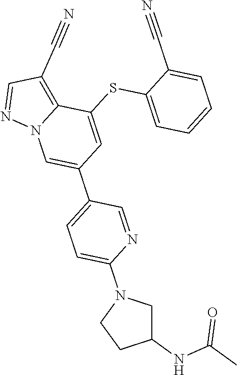 [SMILES: CC(=O)NC1CCN(c2ccc(-c3cc(Sc4ccccc4C#N)c4c(C#N)cnn4c3)cn2)C1] | N-(1-(5-(3-cyano-4-((2- cyanophenyl)thio)pyrazolo [1,5-a]pyridin-6- yl)pyridin-2-yl)pyrrolidin- 3-yl)acetamide | Example 39 | 480.0 | 1 |
| 563 | 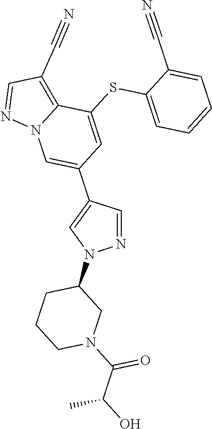 [SMILES: C[C@@H](O)C(=O)N1CCC[C@@H](n2cc(-c3cc(Sc4ccccc4C#N)c4c(C#N)cnn4c3)cn2)C1] | 4-((2-cyanophenyl)thio)- 6-(1-((R)-1-((R)-2- hydroxypropanoyl)piperidin- 3-yl)-1H-pyrazol-4- yl)pyrazolo[1,5- a]pyridine-3-carbonitrile | Example 36 | 498.3 | 1 |
| 564 | 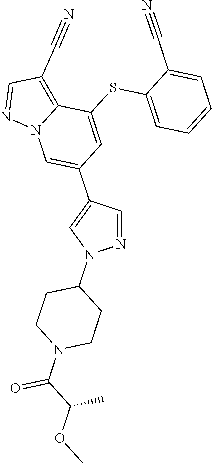 [SMILES: CO[C@@H](C)C(=O)N1CCC(n2cc(-c3cc(Sc4ccccc4C#N)c4c(C#N)cnn4c3)cn2)CC1] | (S)-4-((2- cyanophenyl)thio)-6-(1- (1-(2- methoxypropanoyl)piperidin- 4-yl)-1H-pyrazol-4- yl)pyrazolo[1,5- a]pyridine-3-carbonitrile | Example 36 | 512.4 | 0.5 |
| 565 | 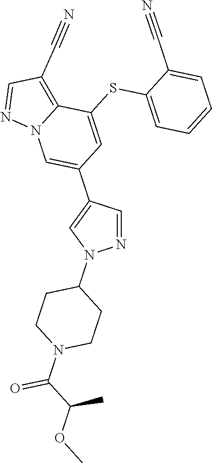 [SMILES: CO[C@H](C)C(=O)N1CCC(n2cc(-c3cc(Sc4ccccc4C#N)c4c(C#N)cnn4c3)cn2)CC1] | (R)-4-((2- cyanophenyl)thio)-6-(1- (1-(2- methoxypropanoyl)piperidin- 4-yl)-1H-pyrazol-4- yl)pyrazolo[1,5- a]pyridine-3-carbonitrile | Example 36 | 512.1 | 0.5 |
| 566 | 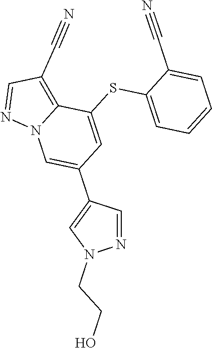 [SMILES: N#Cc1ccccc1Sc1cc(-c2cnn(CCO)c2)cn2ncc(C#N)c12] | 4-((2-cyanophenyl)thio)- 6-(1-(2-hydroxyethyl)-1H- pyrazol-4-yl)pyrazolo[1,5- a]pyridine-3-carbonitrile | Example 35 | 387.2 | 2 |
| 567 | 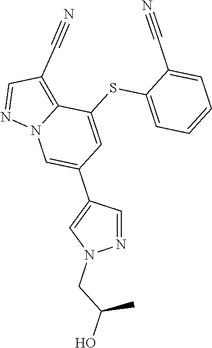 [SMILES: C[C@@H](O)Cn1cc(-c2cc(Sc3ccccc3C#N)c3c(C#N)cnn3c2)cn1] | (R)-4-((2- cyanophenyl)thio)-6-(1- (2-hydroxypropyl)-1H- pyrazol-4-yl)pyrazolo[1,5- a]pyridine-3-carbonitrile | Example 35 | 401.1 | 1 |
| 568 | 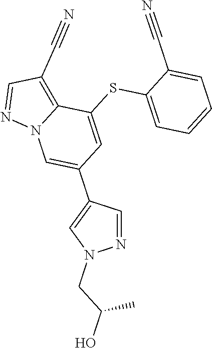 [SMILES: C[C@H](O)Cn1cc(-c2cc(Sc3ccccc3C#N)c3c(C#N)cnn3c2)cn1] | (S)-4-((2- cyanophenyl)thio)-6-(1- (2-hydroxypropyl)-1H- pyrazol-4-yl)pyrazolo[1,5- a]pyridine-3-carbonitrile | Example 35 | 401.1 | 1 |
| 569 | 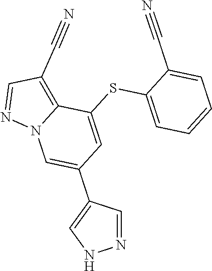 [SMILES: N#Cc1ccccc1Sc1cc(-c2cn[nH]c2)cn2ncc(C#N)c12] | 4-((2-cyanophenyl)thio)- 6-(1H-pyrazol-4- yl)pyrazolo[1,5- a]pyridine-3-carbonitrile | Example 53 | 343.0 | 2 |
| 570 | 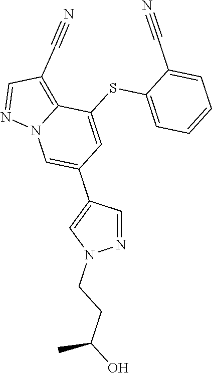 [SMILES: C[C@H](O)CCn1cc(-c2cc(Sc3ccccc3C#N)c3c(C#N)cnn3c2)cn1] | (S)-4-((2- cyanophenyl)thio)-6-(1- (3-hydroxybutyl)-1H- pyrazol-4-yl)pyrazolo∥,5- a]pyridine-3-carbonitrile | Example 35 | 415.3 | 1 |
| 571 | 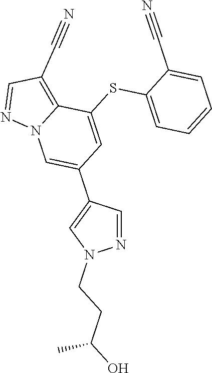 [SMILES: C[C@@H](O)CCn1cc(-c2cc(Sc3ccccc3C#N)c3c(C#N)cnn3c2)cn1] | (R)-4-((2- cyanophenyl)thio)-6-(1- (3-hydroxybutyl)-1H- pyrazol-4-yl)pyrazolo[1,5- a]pyridine-3-carbonitrile | Example 35 | 415.0 | 1 |
| 572 | 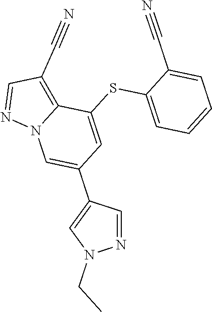 [SMILES: CCn1cc(-c2cc(Sc3ccccc3C#N)c3c(C#N)cnn3c2)cn1] | 4-((2-cyanophenyl)thio)- 6-(1-ethyl-1H-pyrazol-4- yl)pyrazolo[1,5- a]pyridine-3-carbonitrile | Example 35 | 371.2 | 1 |
| 573 | 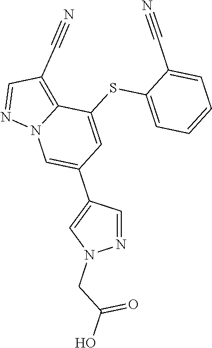 [SMILES: N#Cc1ccccc1Sc1cc(-c2cnn(CC(=O)O)c2)cn2ncc(C#N)c12] | 2-(4-(3-cyano-4-((2- cyanophenyl)thio)pyrazolo [1,5-a]pyridin-6-yl)-1H- pyrazol-1-yl)acetic acid | Example 35 | 401.2 | 4 |
| 574 | 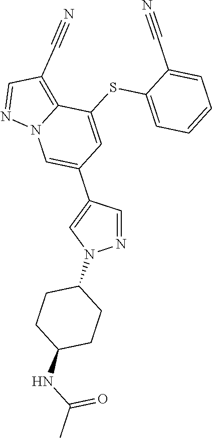 [SMILES: CC(=O)N[C@H]1CC[C@H](n2cc(-c3cc(Sc4ccccc4C#N)c4c(C#N)cnn4c3)cn2)CC1] | N-((1r,4r)-4-(4-(3-cyano- 4-((2- cyanophenyl)thio)pyrazolo [1,5-a]pyridin-6-yl)-1H- pyrazol-1- yl)cyclohexyl)acetamide | Example 39 | 482.3 | 1 |
| 575 | 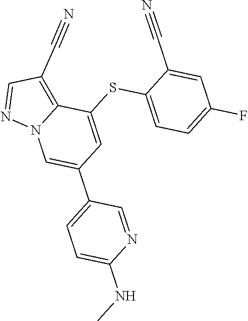 [SMILES: CNc1ccc(-c2cc(Sc3ccc(F)cc3C#N)c3c(C#N)cnn3c2)cn1] | 4-((2-cyano-4- fluorophenyl)thio)-6-(6- (methylamino)pyridin-3- yl)pyrazolo[1,5- a]pryidine-3-carbonitrile | Example 53 | 401.0 | 5 |
| 576 | 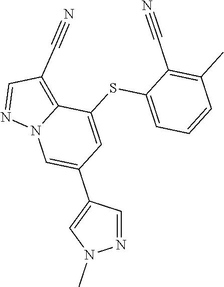 [SMILES: Cc1cccc(Sc2cc(-c3cnn(C)c3)cn3ncc(C#N)c23)c1C#N] | 4-((2-cyano-3- methylphenyl)thio)-6-(1- methyl-1H-pyrazol-4- yl)pyrazolo[1,5- a]pyridine-3-carbonitrile | Example 53 | 371.2 | 7 |
| 577 | 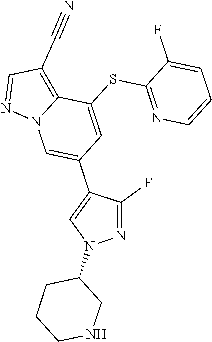 [SMILES: N#Cc1cnn2cc(-c3cn([C@H]4CCCNC4)nc3F)cc(Sc3ncccc3F)c12] | (S)-6-(3-fluoro-1- (piperidin-3-yl)-1H- pyrazol-4-yl)-4-((3- fluoropyridin-2- yl)thio)pyrazolo[1,5- a]pyridine-3-carbonitrile | Example 53 | 438.1 | 7 |
| 578 | 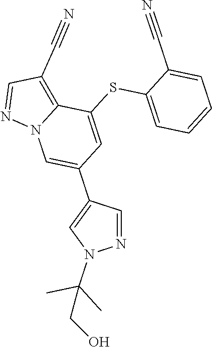 [SMILES: CC(C)(CO)n1cc(-c2cc(Sc3ccccc3C#N)c3c(C#N)cnn3c2)cn1] | 4-((2-cyanophenyl)thio)- 6-(1-(1-hydroxy-2- methylpropan-2-yl)-1H- pyrazol-4-yl)pyrazolo[1,5- a]pyridine-3-carbonitrile | Example 35 | 415.1 | 2 |
| 579 | 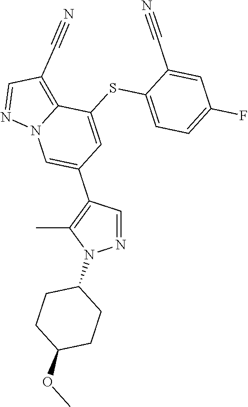 [SMILES: CO[C@H]1CC[C@H](n2ncc(-c3cc(Sc4ccc(F)cc4C#N)c4c(C#N)cnn4c3)c2C)CC1] | 4-((2-cyano-4- fluorophenyl)thio)-6-(1- ((1r,4r)-4- methoxycyclohexyl)-5- methyl-1H-pyrazol-4- yl)pyrazolo[1,5- a]pyridine-3-carbonitrile | Example 53 | 487.2 | 1.5 |
| 580 | 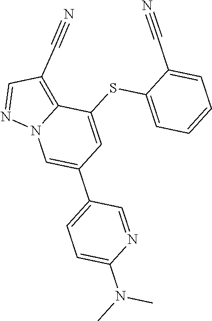 [SMILES: CN(C)c1ccc(-c2cc(Sc3ccccc3C#N)c3c(C#N)cnn3c2)cn1] | 4-((2-cyanophenyl)thio)- 6-(6- (dimethylamino)pyridin- 3-yl)pyrazolo[1,5- a]pyridine-3-carbonitrile | Example 53 | 397.1 | 1 |
| 581 | 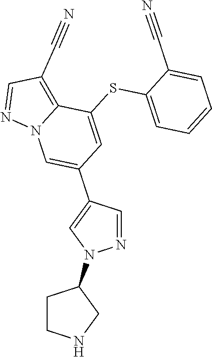 [SMILES: N#Cc1ccccc1Sc1cc(-c2cnn([C@@H]3CCNC3)c2)cn2ncc(C#N)c12] | (R)-4-((2- cyanophenyl)thio)-6-(1- (pyrrolidin-3-yl)-1H- pyrazol-4-yl)pyrazolo[1,5- a]pyridine-3-carbonitrile | Example 53 | 412.3 | 0.5 |
| 582 | 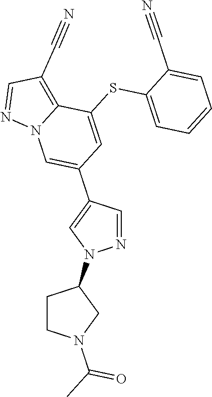 [SMILES: CC(=O)N1CC[C@@H](n2cc(-c3cc(Sc4ccccc4C#N)c4c(C#N)cnn4c3)cn2)C1] | (R)-6-(1-(1- acetylpyrrolidin-3-yl)-1H- pyrazol-4-yl)-4-((2- cyanophenyl)thio)pyrazolo [1,5-a]pyridine-3- carbonitrile | Example 39 | 454.2 | 1 |
| 583 | 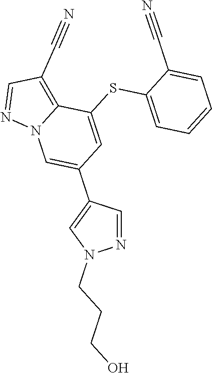 [SMILES: N#Cc1ccccc1Sc1cc(-c2cnn(CCCO)c2)cn2ncc(C#N)c12] | 4-((2-cyanophenyl)thio)- 6-(1-(3-hydroxypropyl)- 1H-pyrazol-4- yl)pyrazolo[1,5- a]pyridine-3-carbonitrile | Example 35 | 401.2 | 1 |
| 584 | 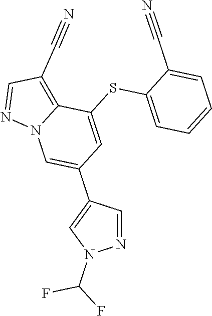 [SMILES: N#Cc1ccccc1Sc1cc(-c2cnn(C(F)F)c2)cn2ncc(C#N)c12] | 4-((2-cyanophenyl)thio)- 6-(1-(difluoromethyl)-1H- pyrazol-4-yl)pyrazolo[1,5- a]pyridine-3-carbonitrile | Example 53 | 393.0 | 6 |
| 585 | 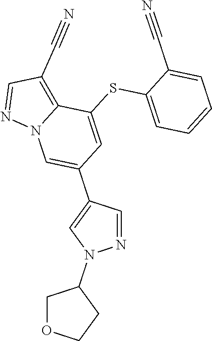 [SMILES: N#Cc1ccccc1Sc1cc(-c2cnn(C3CCOC3)c2)cn2ncc(C#N)c12] | 4-((2-cyanophenyl)thio)- 6-(1-(tetrahydrofuran-3- yl)-1H-pyrazol-4- yl)pyrazolo[1,5- a]pryidine-3-carbonitrile | Example 35 | 413.1 | 1 |
| 586 | 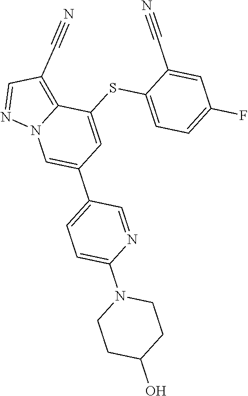 [SMILES: N#Cc1cc(F)ccc1Sc1cc(-c2ccc(N3CCC(O)CC3)nc2)cn2ncc(C#N)c12] | 4-((2-cyano-4- fluorophenyl)thio)-6-(6- (4-hydroxypiperidin-1- yl)pyridin-3- yl)pyrazolo[1,5- a]pyridine-3-carbonitrile | Example 53 | 471.1 | 1 |
| 587 | 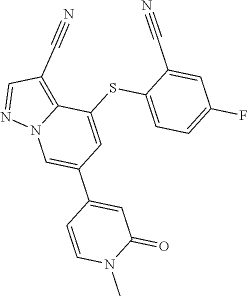 [SMILES: Cn1ccc(-c2cc(Sc3ccc(F)cc3C#N)c3c(C#N)cnn3c2)cc1=O] | 4-((2-cyano-4- fluorophenyl)thio)-6-(1- methyl-2-oxo-1,2- dihydropyridin-4- yl)pyrazolo[1,5- a]pyridine-3-carbonitrile | Example 53 | 402.0 | 43 |
| 588 | 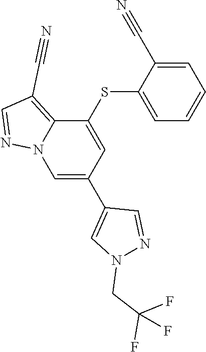 [SMILES: N#Cc1ccccc1Sc1cc(-c2cnn(CC(F)(F)F)c2)cn2ncc(C#N)c12] | 4-((2-cyano-4- fluorophenyl)thio)-6-(1- methyl-2-oxo-1,2- dihydropyridin-4- yl)pyrazolo[1,5- a]pyridine-3-carbonitrile | Example 35 | 425.1 | 2 |
| 589 | 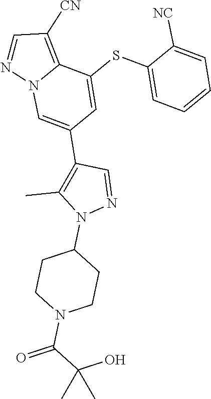 [SMILES: Cc1c(-c2cc(Sc3ccccc3C#N)c3c(C#N)cnn3c2)cnn1C1CCN(C(=O)C(C)(C)O)CC1] | 4-((2-cyanophenyl)thio)- 6-(1-(1-(2-hydroxy-2- methylpropanoyl)piperidin- 4-yl)-5-methyl-1H- pyrazol-4-yl)pyrazolo[1,5- a]pyridine-3-carbonitrile | Example 36 | 526.2 | 1 |
| 590 | 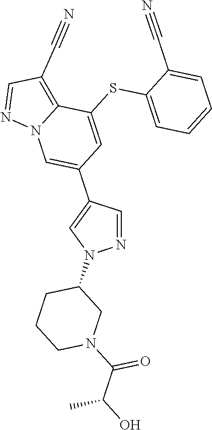 [SMILES: C[C@@H](O)C(=O)N1CCC[C@H](n2cc(-c3cc(Sc4ccccc4C#N)c4c(C#N)cnn4c3)cn2)C1] | 4-((2-cyanophenyl)thio)- 6-(1-((S)-1-((R)-2- hydroxypropanoyl)piperidin- 3-yl)-1H-pyrazol-4- yl)pyrazolo[1,5- a]pyridine-3-carbonitrile | Example 30 | 498.4 | 0.5 |
| 591 | 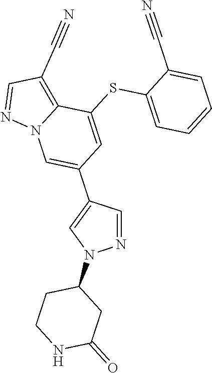 [SMILES: N#Cc1ccccc1Sc1cc(-c2cnn([C@@H]3CCNC(=O)C3)c2)cn2ncc(C#N)c12] | (R)-4-((2- cyanophenyl)thio)-6-(1- (2-oxopiperidin-4-yl)-1H- pyrazol-4-yl)pyrazolo[1,5- a]pyridine-3-carbonitrile | Example 53 | 440.1 | 0.4 |
| 592 | 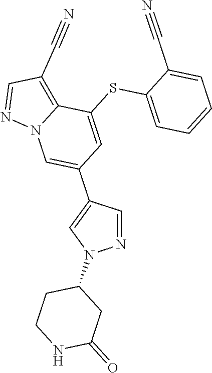 [SMILES: N#Cc1ccccc1Sc1cc(-c2cnn([C@H]3CCNC(=O)C3)c2)cn2ncc(C#N)c12] | (S)-4-((2- cyanophenyl)thio)-6-(1- (2-oxopiperidin-4-yl)-1H- pyrazol-4-yl)pyrazolo[1,5- a]pyridine-3-carbonitrile | Example 53 | 440.1 | 1 |
| 593 | 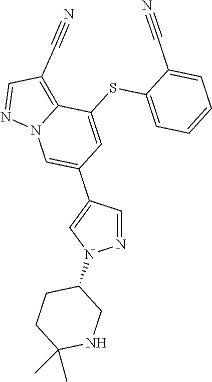 [SMILES: CC1(C)CC[C@H](n2cc(-c3cc(Sc4ccccc4C#N)c4c(C#N)cnn4c3)cn2)CN1] | (S)-4-((2- cyanophenyl)thio)-6-(1- (6,6-dimethylpiperidin-3- yl)-1H-pyrazol-4- yl)pyrazolo[1,5- a]pyridine-3-carbonitrile | Example 64 | 454.2 | 1 |
| 594 | 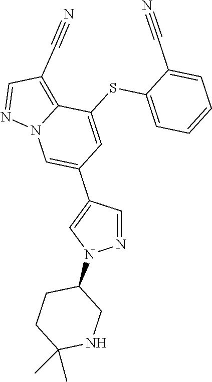 [SMILES: CC1(C)CC[C@@H](n2cc(-c3cc(Sc4ccccc4C#N)c4c(C#N)cnn4c3)cn2)CN1] | (R)-4-((2- cyanophenyl)thio)-6-(1- (6,6-dimethylpiperidin-3- yl)-1H-pyrazol-4- yl)pyrazolo[1,5- a]pyridine-3-carbonitrile | Example 64 | 454.1 | 1 |
| 595 | 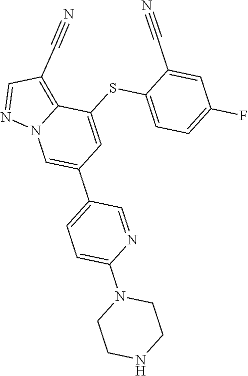 [SMILES: N#Cc1cc(F)ccc1Sc1cc(-c2ccc(N3CCNCC3)nc2)cn2ncc(C#N)c12] | 4-((2-cyano-4- fluorophenyl)thio)-6-(6- (piperazin-1-yl)pyridin-3- yl)pyrazolo[1,5- a]pyridine-3-carbonitrile | Example 53 | 456.1 | 15 |
| 596 | 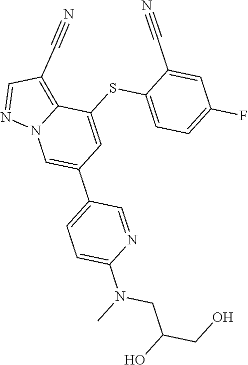 [SMILES: CN(CC(O)CO)c1ccc(-c2cc(Sc3ccc(F)cc3C#N)c3c(C#N)cnn3c2)cn1] | 4-((2-cyano-4- fluorophenyl)thio)-6-(6- ((2,3- dihydroxypropyl)(methyl) amino)pyridin-3- yl)pyrazolo[1,5- a]pyridine-3-carbonitrile | Example 60 | 475.3 | 1 |
| 597 | 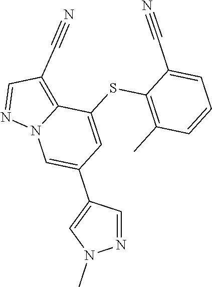 [SMILES: Cc1cccc(C#N)c1Sc1cc(-c2cnn(C)c2)cn2ncc(C#N)c12] | 4-((2-cyano-6- methylphenyl)thio)-6-(1- methyl-1H-pyrazol-4- yl)pyrazolo[1,5- a]pryidine-3-carbonitrile | Example 53 | 371.2 | 30 |
| 598 | 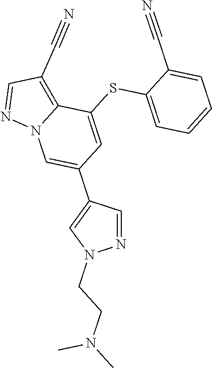 [SMILES: CN(C)CCn1cc(-c2cc(Sc3ccccc3C#N)c3c(C#N)cnn3c2)cn1] | 4-((2-cyanophenyl)thio)- 6-(1-(2- (dimethylamino)ethyl)- 1H-pyrazol-4- yl)pyrazolo[1,5- a]pryidine-3-carbontrile | Example 35 | 414.2 | 1 |
| 599 | 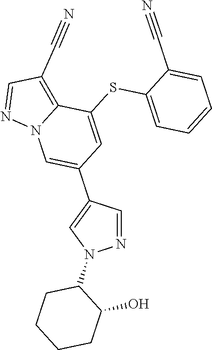 | 4-((2-cyanophenyl)thio)- 6-(1-((1S,2R)-2- hydroxycyclohexyl)-1H- pyrazol-4-yl)pyrazolo[1,5- a]pyridine-3-carbonitrile | Example 35 | 441.1 | 2 |
| 600 | 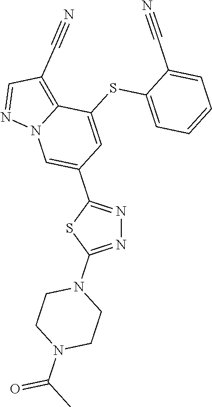 [SMILES: CC(=O)N1CCN(c2nnc(-c3cc(Sc4ccccc4C#N)c4c(C#N)cnn4c3)s2)CC1] | 6-(5-(4-acetylpiperazin-1- y)-1,3,4-thiadiazol-2-yl)- 4-((2- cyanophenyl)thio)pyrazolo [1,5-a]pyridine-3- carbonitrile | Example 39 | 487.2 | 6 |
| 601 | 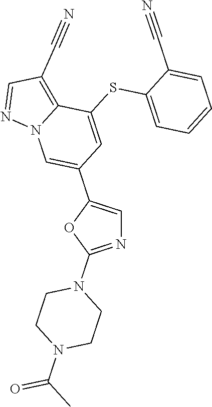 [SMILES: CC(=O)N1CCN(c2ncc(-c3cc(Sc4ccccc4C#N)c4c(C#N)cnn4c3)o2)CC1] | 6-(2-(4-acetylpiperazin-1- yl)oxazol-5-yl)-4-((2- cyanophenyl)thio)pyrazolo [1,5-a]pyridine-3- carbonitrile | Example 39 | 470.1 | 53 |
| 602 | 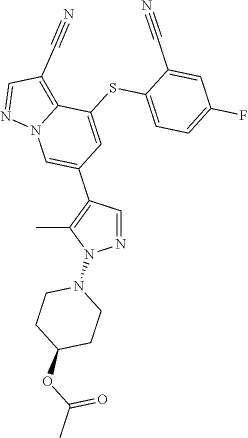 | (1r,4r)-4-(4-(3-cyano-4- ((2-cyano-4- fluorophenyl)thhio)pyrazolo [1,5-a]pyridin-6-yl)-5- methyl-1H-pyrazol-1- yl)cyclohexyl acetate | Example 39 | 515.2 | 2 |
| 603 | 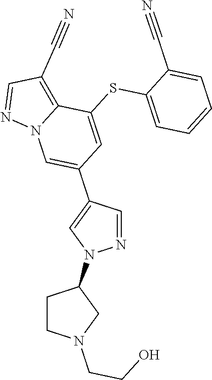 [SMILES: N#Cc1ccccc1Sc1cc(-c2cnn([C@@H]3CCN(CCO)C3)c2)cn2ncc(C#N)c12] | (R)-4-((2- cyanophenyl)thio)-6-(1- (1-(2- hydroxyethyl)pyrrolidin- 3-yl)-1H-pyrazol-4- yl)pyrazolo[1,5- a]pyridine-3-carbonitrile | Example 35 | 456.2 | 0.6 |
| 604 | 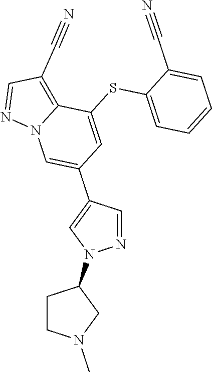 [SMILES: CN1CC[C@@H](n2cc(-c3cc(Sc4ccccc4C#N)c4c(C#N)cnn4c3)cn2)C1] | (R)-4-((2- cyanophenyl)thio)-6-(1- (1-methylpyrrolidin-3-yl)- 1H-pyrazol-4- yl)pyrazolo[1,5- a]pyridine-3-carbonitrile | Example 53 | 426.2 | 0.5 |
| 605 | 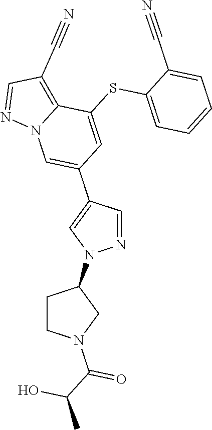 [SMILES: C[C@@H](O)C(=O)N1CC[C@@H](n2cc(-c3cc(Sc4ccccc4C#N)c4c(C#N)cnn4c3)cn2)C1] | 4-((2-cyanophenyl)thio)- 6-(1-((R)-1-((R)-2- hydroxypropanoyl)pyrroli- din-3-yl)-1H-pyrazol-4- yl)pyrazolo[1,5- a]pyridine-3-carbonitrile | Example 36 | 484.1 | 0.6 |
| 606 | 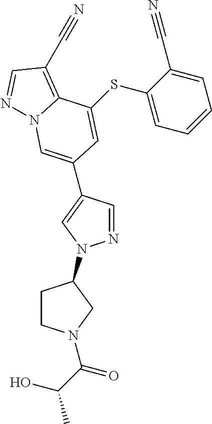 [SMILES: C[C@H](O)C(=O)N1CC[C@@H](n2cc(-c3cc(Sc4ccccc4C#N)c4c(C#N)cnn4c3)cn2)C1] | 4-((2-cyanophenyl)thio)- 6-(1-((R)-1-((S)-2- hydroxypropanoyl)pyrroli din-3-yl)-1H-pyrazol-4- y)pyrazolo[1,5- a]pyridine-3-carbonitrile | Example 36 | 484.2 | 0.5 |
| 607 | 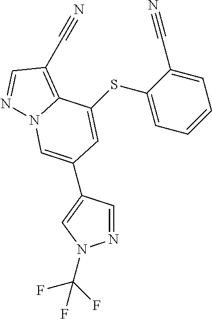 [SMILES: N#Cc1ccccc1Sc1cc(-c2cnn(C(F)(F)F)c2)cn2ncc(C#N)c12] | 4-((2-cyanophenyl)thio)- 6-(1-(trifluoromethyl)-1H- pyrazol-4-yl)pyrazolo[1,5- a]pyridine-3-carbonitrile | Example 53 | 411.1 | 10 |
| 608 | 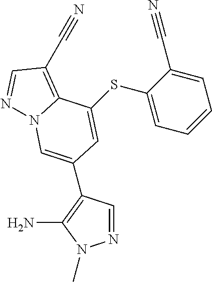 [SMILES: Cn1ncc(-c2cc(Sc3ccccc3C#N)c3c(C#N)cnn3c2)c1N] | 6-(5-amino-1-methyl-1H- pyrazol-4-yl)-4-((2- cyanophenyl)thio)pyrazolo [1,5-a]pyridine-3- carbonitrile | Example 67 | 372.1 | 2 |
| 609 | 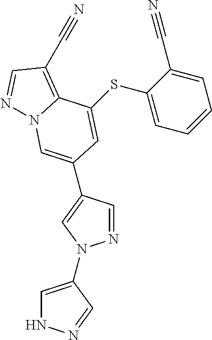 [SMILES: N#Cc1ccccc1Sc1cc(-c2cnn(-c3cn[nH]c3)c2)cn2ncc(C#N)c12] | 6-(1′H-[1,4′-bipyrazol]-4- yl)-4-((2- cyanophenyl)thio)pyrazolo [1,5-a]pyridine-3- carbonitrile | Example 53 | 409.1 | 4 |
| 610 | 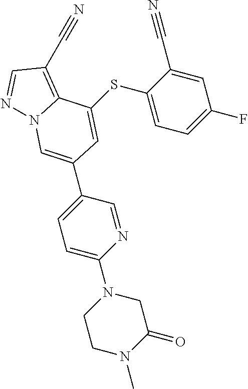 [SMILES: CN1CCN(c2ccc(-c3cc(Sc4ccc(F)cc4C#N)c4c(C#N)cnn4c3)cn2)CC1=O] | 4-((2-cyano-4- fluorophenyl)thio)-6-(6- (4-methyl-3-oxopiperazin-1- yl)pyridin-3- yl)pyrazolo[1,5- a]pyridine-3-carbonitrile | Example 60 | 484.1 | 2 |
| 611 | 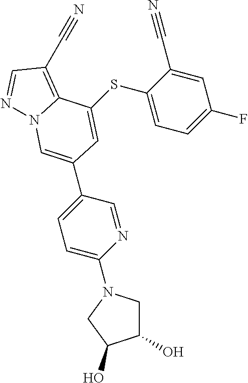 [SMILES: N#Cc1cc(F)ccc1Sc1cc(-c2ccc(N3C[C@H](O)[C@@H](O)C3)nc2)cn2ncc(C#N)c12] | 4-((2-cyano-4- fluorophenyl)thio)-6-(6- ((3S,4S)-3,4- dihydroxypyrrolidin-1- yl)pyridin-3- yl)pyrazolo[1,5- a]pyridine-3-carbonitrile | Example 60 | 473.1 | 3 |
| 612 | 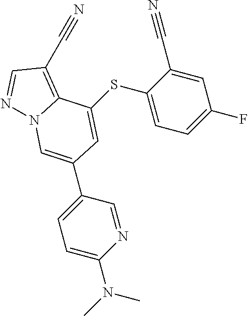 [SMILES: CN(C)c1ccc(-c2cc(Sc3ccc(F)cc3C#N)c3c(C#N)cnn3c2)cn1] | 4-((2-cyano-4- fluorophenyl)thio)-6-(6- (dimethylamino)pyridin- 3-yl)pyrazolo[1,5- a]pyridine-3-carbonitrile | Example 53 | 415.1 | 2 |
| 613 | 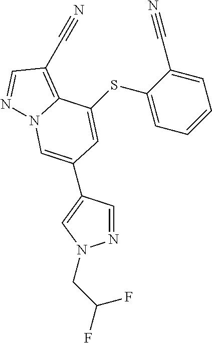 [SMILES: N#Cc1ccccc1Sc1cc(-c2cnn(CC(F)F)c2)cn2ncc(C#N)c12] | 4-((2-cyanophenyl)thio)- 6-(1-(2,2-difluoroethyl)- 1H-pyrazol-4- yl)pyrazolo[1,5- a]pyridine-3-carbonitrile | Example 35 | 407.0 | 1 |
| 614 | 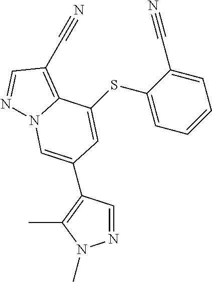 [SMILES: Cc1c(-c2cc(Sc3ccccc3C#N)c3c(C#N)cnn3c2)cnn1C] | 4-((2-cyanophenyl)thio)- 6-(1,5-dimethyl-1H- pyrazol-4-yl)pyrazolo[1,5- a]pyridine-3-carbonitrile | Example 53 | 371.1 | 9 |
| 615 | 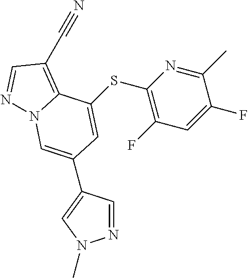 [SMILES: Cc1nc(Sc2cc(-c3cnn(C)c3)cn3ncc(C#N)c23)c(F)cc1F] | 4-((3,5-difluoro-6- methylpyridin-2-yl)thio)- 6-(1-methyl-1H-pyrazol- 4-yl)pyrazolo[1,5- a]pyridine-3-carbonitrile | Example 53 | 383.1 | 2 |
| 616 | 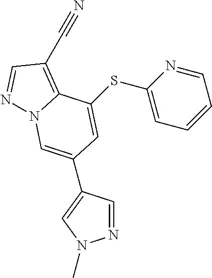 [SMILES: Cn1cc(-c2cc(Sc3ccccn3)c3c(C#N)cnn3c2)cn1] | 6-(1-methyl-1H-pyrazol- 4-yl)-4-(pyridin-2- ylthio)pyrazolo[1,5- a]pyridine-3-carbonitrile | Example 53 | 333.2 | 6 |
| 617 | 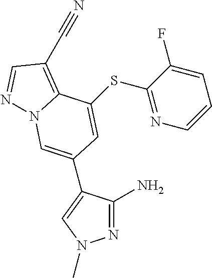 [SMILES: Cn1cc(-c2cc(Sc3ncccc3F)c3c(C#N)cnn3c2)c(N)n1] | 6-(3-amino-1-methyl-1H- pyrazol-4-yl)-4-((3- fluoropyridin-2- yl)thio)pyrazolo[1,5- a]pyridine-3-carbonitrile | Example 53 | 366.0 | 6 |
| 618 | 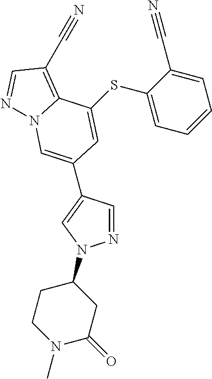 [SMILES: CN1CC[C@@H](n2cc(-c3cc(Sc4ccccc4C#N)c4c(C#N)cnn4c3)cn2)CC1=O] | (R)-4-((2- cyanophenyl)thio)-6-(1- (1-methyl-2-oxopiperidin- 4-yl)-1H-pyrazol-4- yl)pyrazolo[1,5- a]pyridine-3-carbonitrile | Example 53 | 454.2 | 0.4 |
| 619 | 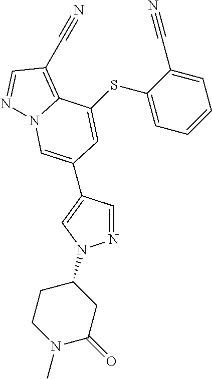 [SMILES: CN1CC[C@H](n2cc(-c3cc(Sc4ccccc4C#N)c4c(C#N)cnn4c3)cn2)CC1=O] | (S)-4-((2- cyanophenyl)thio)-6-(1- (1-methyl-2-oxopiperidin- 4-yl)-1H-pyrazol-4- yl)pyrazolo[1,5- a]pyridine-3-carbonitrile | Example 53 | 454.1 | 2 |
| 620 | 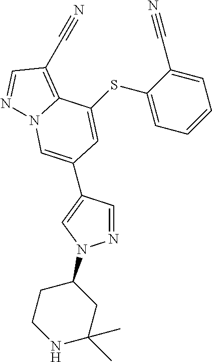 [SMILES: CC1(C)C[C@H](n2cc(-c3cc(Sc4ccccc4C#N)c4c(C#N)cnn4c3)cn2)CCN1] | (R)-4-((2- cyanophenyl)thio)-6-(1- (2,2-dimethylpiperidin-4- yl)-1H-pyrazol-4- yl)pyrazolo[1,5- a]pyridine-3-carbonitrile | Example 53 | 454.3 | 0.3 |
| 621 | 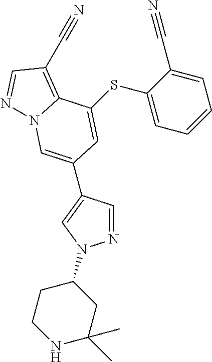 [SMILES: CC1(C)C[C@@H](n2cc(-c3cc(Sc4ccccc4C#N)c4c(C#N)cnn4c3)cn2)CCN1] | (S)-4-((2- cyanophenyl)thio)-6-(1- (2,2-dimethylpiperidin-4- yl)-1H-pyrazol-4- yl)pyrazolo[1,5- a]pyridine-3-carbonitrile | Example 53 | 454.3 | 0.3 |
| 622 | 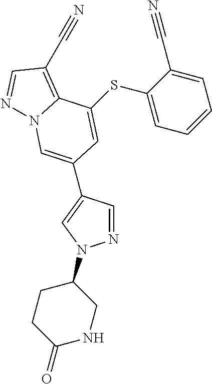 [SMILES: N#Cc1ccccc1Sc1cc(-c2cnn([C@@H]3CCC(=O)NC3)c2)cn2ncc(C#N)c12] | (R)-4-((2- cyanophenyl)thio)-6-(1- (6-oxopiperidin-3-yl)-1H- pyrazol-4-yl)pyrazolo[1,5- a]pyridine-3-carbonitrile | Example 35 | 440.1 | 0.5 |
| 623 | 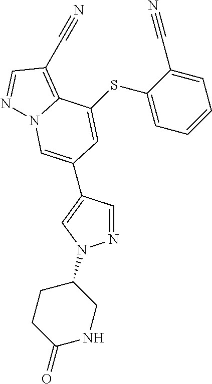 [SMILES: N#Cc1ccccc1Sc1cc(-c2cnn([C@H]3CCC(=O)NC3)c2)cn2ncc(C#N)c12] | (S)-4-((2- cyanophenyl)thio)-6-(1- (6-oxopiperidin-3-yl)-1H- pyrazol-4-yl)pyrazolo[1,5- a]pyridine-3-carbonitrile | Example 35 | 440.1 | 1 |
| 624 | 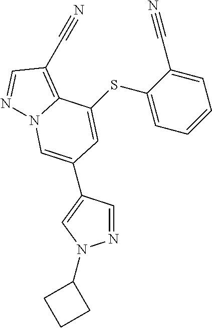 [SMILES: N#Cc1ccccc1Sc1cc(-c2cnn(C3CCC3)c2)cn2ncc(C#N)c12] | 4-((2-cyanophenyl)thio)- 6-(1-cyclobutyl-1H- pyrazol-4-yl)pyrazolo[1,5- a]pyridine-3-carbonitrile | Example 35 | 397.3 | 1 |
| 625 | 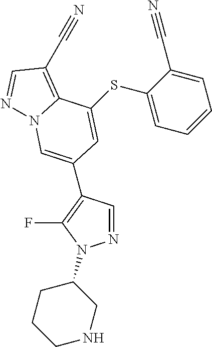 [SMILES: N#Cc1ccccc1Sc1cc(-c2cnn([C@H]3CCCNC3)c2F)cn2ncc(C#N)c12] | (S)-4-((2- cyanophenyl)thio)-6-(5- fluoro-1-(piperidin-3-yl)- 1H-pyrazol-4- yl)pyrazolo[1,5- a]pyridine-3-carbonitrile | Example 53 | 444.2 | 1 |
| 626 | 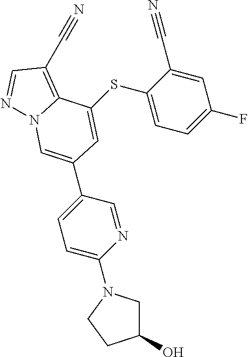 [SMILES: N#Cc1cc(F)ccc1Sc1cc(-c2ccc(N3CC[C@H](O)C3)nc2)cn2ncc(C#N)c12] | (S)-4-((2-cyano-4- fluorophenyl)thio)-6-(6- (3-hydroxypyrrolidin-1- yl)pyridin-3- yl)pyrazolo[1,5- a]pyridine-3-carbonitrile | Example 60 | 457.0 | 1 |
| 627 | 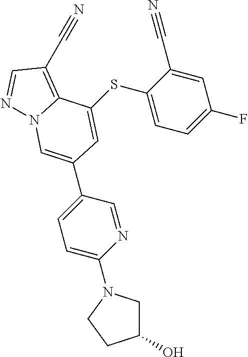 [SMILES: N#Cc1cc(F)ccc1Sc1cc(-c2ccc(N3CC[C@@H](O)C3)nc2)cn2ncc(C#N)c12] | (R)-4-((2-cyano-4- fluorophenyl)thio)-6-(6- (3-hydroxypyrrolidin-1- yl)pyridin-3- yl)pyrazolo[1,5- a]pyridine-3-carbonitrile | Example 60 | 457.2 | 2 |
| 628 | 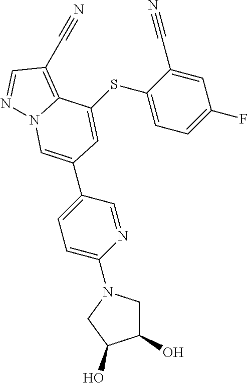 [SMILES: N#Cc1cc(F)ccc1Sc1cc(-c2ccc(N3C[C@@H](O)[C@@H](O)C3)nc2)cn2ncc(C#N)c12] | 4-((2-cyano-4- fluorophenyl)thio)-6-(6- ((3R,4S)-3,4- dihydroxypyrrolidin-1- yl)pyridin-3- yl)pyrazolo[1,5- a]pyridine-3-carbonitrile | Example 60 | 473.1 | 1 |
| 629 | 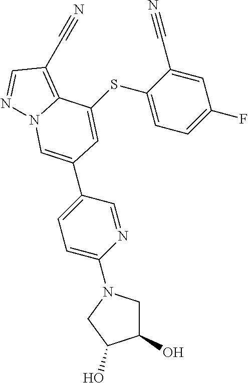 [SMILES: N#Cc1cc(F)ccc1Sc1cc(-c2ccc(N3C[C@@H](O)[C@H](O)C3)nc2)cn2ncc(C#N)c12] | 4-((2-cyano-4- fluorophenyl)thio)-6-(6- ((3R,4R)-3,4- dihydroxypyrrolidin-1- yl)pyridin-3- yl)pyrazolo[1,5- a]pyridine-3-carbonitrile | Example 60 | 473.0 | 1.6 |
| 630 | 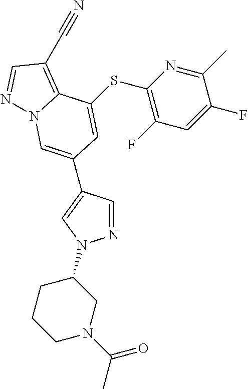 [SMILES: CC(=O)N1CCC[C@H](n2cc(-c3cc(Sc4nc(C)c(F)cc4F)c4c(C#N)cnn4c3)cn2)C1] | (S)-6-(1-(1- acetylpiperidin-3-yl)-1H- pyrazol-4-yl)-4-((3,5- difluoro-6-methylpyridin- 2-yl)thio)pyrazolo[1,5- a]pyridine-3-carbonitrile | Example 53 | 494.1 | 1 |
| 631 | 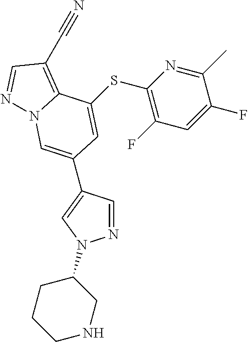 [SMILES: Cc1nc(Sc2cc(-c3cnn([C@H]4CCCNC4)c3)cn3ncc(C#N)c23)c(F)cc1F] | (S)-4-((3,5-difluoro-6- methylpyridin-2-yl)thio)- 6-(1-(piperidin-3-yl)-1H- pyrazol-4-yl)pyrazolo[1,5- a]pyridine-3-carbonitrile | Example 53 | 452.1 | 1 |
| 632 | 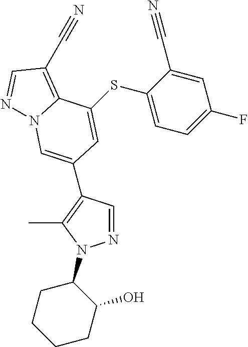 [SMILES: Cc1c(-c2cc(Sc3ccc(F)cc3C#N)c3c(C#N)cnn3c2)cnn1[C@@H]1CCCC[C@H]1O] | 4-((2-cyano-4- fluorophenyl)thio)-6-(1- ((1R,2R)-2- hydroxycyclohexyl)-5- methyl-1H-pyrazol-4- yl)pyrazolo[1,5- a]pyridine-3-carbonitrile | Example 53 | 473.2 | 32 |
| 633 | 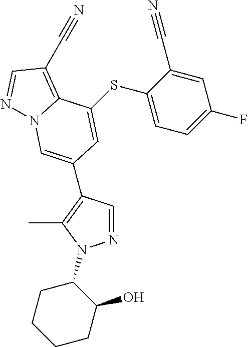 [SMILES: Cc1c(-c2cc(Sc3ccc(F)cc3C#N)c3c(C#N)cnn3c2)cnn1[C@H]1CCCC[C@@H]1O] | 4-((2-cyano-4- fluorophenyl)thio)-6-(1- ((2S,2S)-2- hydroxycyclohexyl)-5- methyl-1H-pyrazol-4- yl)pyrazolo[1,5- a]pyridine-3-carbonitrile | Example 53 | 473.2 | 1 |
| 634 | 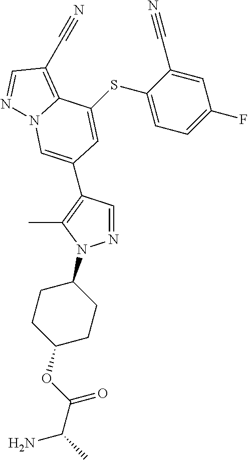 [SMILES: Cc1c(-c2cc(Sc3ccc(F)cc3C#N)c3c(C#N)cnn3c2)cnn1[C@H]1CC[C@H](OC(=O)[C@H](C)N)CC1] | (1r,4S)-4-(4-(3-cyano-4- ((2-cyano-4 fluorophenyl)thio)pyrazolo [1,5-a]pyridin-6-yl)-5- methyl-1H-pyrazol-1- yl)cyclohexyl L-alaninate | Example 36 | 544.3 | 1 |
| 635 | 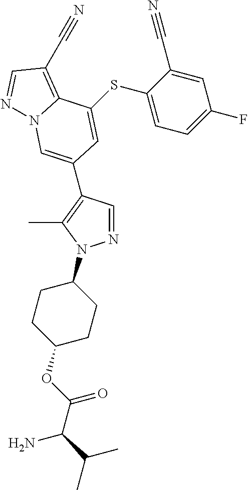 [SMILES: Cc1c(-c2cc(Sc3ccc(F)cc3C#N)c3c(C#N)cnn3c2)cnn1[C@H]1CC[C@H](OC(=O)[C@H](N)C(C)C)CC1] | (1r,4R)-4-(4-(3-cyano-4- ((2-cyano-4- fluorophenyl)thio)pyrazolo [1,5-a]pyridin-6-yl)-5- methyl-1H-pyrazol-1- yl)cyclohexyl D-valinate | Example 36 | 572.3 | 1 |
| 636 | 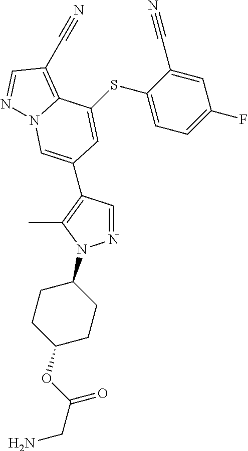 [SMILES: Cc1c(-c2cc(Sc3ccc(F)cc3C#N)c3c(C#N)cnn3c2)cnn1[C@H]1CC[C@H](OC(=O)CN)CC1] | (1r,4r)-4-(4-(3-cyano-4- ((2-cyano-4- fluorophenyl)thio)pyrazolo [1,5-a]pyridin-6-yl)-5- methyl-1H-pyrazol-1- yl)cyclohexyl glycinate | Example 36 | 530.3 | 1 |
| 637 | 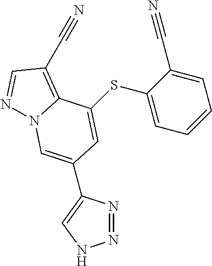 [SMILES: N#Cc1ccccc1Sc1cc(-c2c[nH]nn2)cn2ncc(C#N)c12] | 4-((2-cyanophenyl)thio)- 6-(1H-1,2,3-triazol-4- yl)pyrazolo[1,5- a]pyridine-3-carbonitrile | Example 69 | 344.1 | 12 |
| 638 | 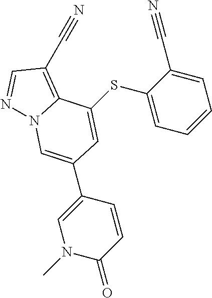 [SMILES: Cn1cc(-c2cc(Sc3ccccc3C#N)c3c(C#N)cnn3c2)ccc1=O] | 4-((2-cyanophenyl)thio)- 6-(1-methyl-6-oxo-1,6- dihydropyridin-3- yl)pyrazolo[1,5- a]pyridine-3-carbonitrile | Example 53 | 384.1 | 18 |
| 639 | 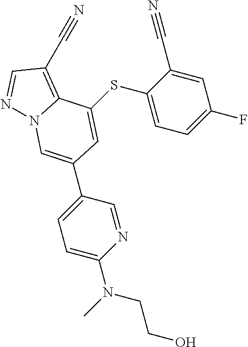 [SMILES: CN(CCO)c1ccc(-c2cc(Sc3ccc(F)cc3C#N)c3c(C#N)cnn3c2)cn1] | 4-((2-cyano-4- fluorophenyl)thio)-6-(6- ((2- hydroxyethyl)(methyl)ami- no)pyridin-3- yl)pyrazolo[1,5- a]pyridine-3-carbonitrile | Example 60 | 4458.1 | 1.5 |
| 640 | 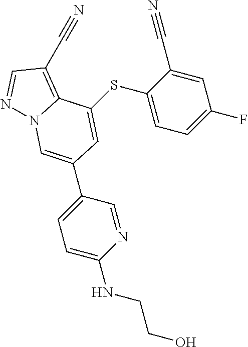 [SMILES: N#Cc1cc(F)ccc1Sc1cc(-c2ccc(NCCO)nc2)cn2ncc(C#N)c12] | 4-((2-cyano-4- fluorophenyl)thio)-6-(6- ((2- hydroxyethyl)amino)pyridin- 3-yl)pyrazolo[1,5- a]pyridine-3-carbonitrile | Example 60 | 431.2 | 2.5 |
| 641 | 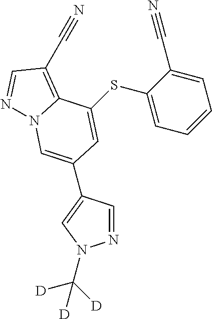 [SMILES: [2H]C([2H])([2H])n1cc(-c2cc(Sc3ccccc3C#N)c3c(C#N)cnn3c2)cn1] | 4-((2-cyanophenyl)thio)- 6-(1-(methyl-d3)-1H- pyrazol-4-yl)pyrazolo[1,5- a]pyridine-3-carbonitrile | Example 53 | 360.0 | 1 |
| 642 | 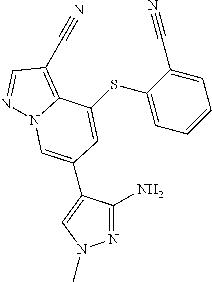 [SMILES: Cn1cc(-c2cc(Sc3ccccc3C#N)c3c(C#N)cnn3c2)c(N)n1] | 6-(3-amino-1-methyl-1H- pyrazol-4-yl)-4-((2- cyanophenyl)thio)pyrazolo [1,5-a]pyridine-3- carbonitrile | Example 53 | 372.0 | 2 |
| 643 | 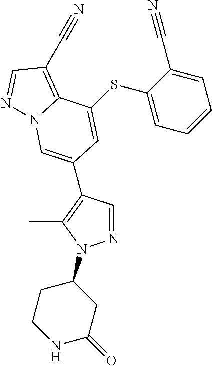 [SMILES: Cc1c(-c2cc(Sc3ccccc3C#N)c3c(C#N)cnn3c2)cnn1[C@@H]1CCNC(=O)C1] | (R)-4-((2- cyanophenyl)thio)-6-(5- methyl-1-(2-oxopiperidin- 4-yl)-1H-pyrazol-4- yl)pyrazolo[1,5- a]pyridine-3-carbonitrile | Example 53 | 454.2 | 3 |
| 644 | 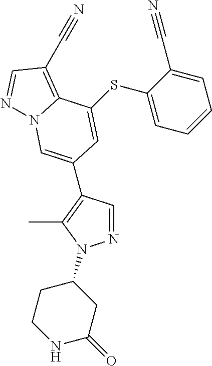 [SMILES: Cc1c(-c2cc(Sc3ccccc3C#N)c3c(C#N)cnn3c2)cnn1[C@H]1CCNC(=O)C1] | 4-((2-cyano-4- fluorophenyl)thio)-6-(6- (pyrrolidin-1-yl)pyridin-3- yl)pyrazolo[1,5- a]pyridine-3-carbonitrile | Example 53 | 454.1 | 1.6 |
| 645 | 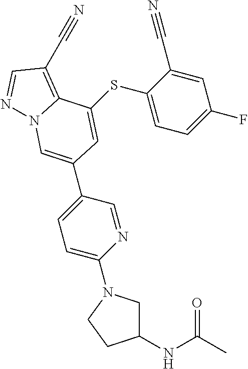 [SMILES: CC(=O)NC1CCN(c2ccc(-c3cc(Sc4ccc(F)cc4C#N)c4c(C#N)cnn4c3)cn2)C1] | (R)-4-((2-cyano-4- fluorophenyl)thio)-6-(1- (2,3-dihydroxypropyl)-5- methyl-1H-pyrazol-4- yl)pyrazolo[1,5- a]pyridine-3-carbonitrile | Example 39 | 498.3 | 1.6 |
| 646 | 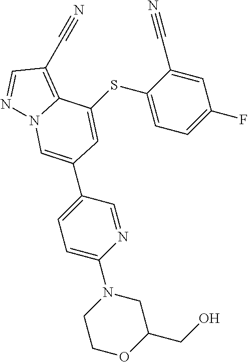 [SMILES: N#Cc1cc(F)ccc1Sc1cc(-c2ccc(N3CCOC(CO)C3)nc2)cn2ncc(C#N)c12] | 4-((2-cyano-4- fluorophenyl)thio)-6-(6- (2- (hydroxymethyl)morpholi- no)pyridin-3- yl)pyrazolo[1,5- a]pyridine-3-carbonitrile | Example 60 | 487.2 | 1 |
| 647 | 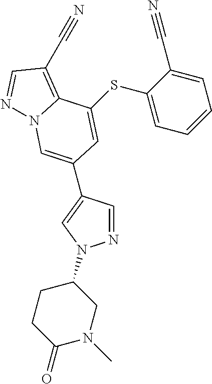 [SMILES: CN1C[C@@H](n2cc(-c3cc(Sc4ccccc4C#N)c4c(C#N)cnn4c3)cn2)CCC1=O] | (S)-4-((2- cyanophenyl)thio)-6-(1- (1-methyl-6-oxopiperidin- 3-yl)-1H-pyrazol-4- yl)pyrazolo[1,5- a]pyridine-3-carbonitrile | Example 60 | 454.1 | 1 |
| 648 | 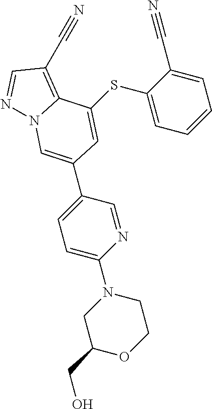 [SMILES: N#Cc1ccccc1Sc1cc(-c2ccc(N3CCO[C@@H](CO)C3)nc2)cn2ncc(C#N)c12] | (R)-4-((2- cyanophenyl)thio)-6-(6- (2- (hydroxymethyl)morpholi- no)pyridin-3- yl)pyrazolo[1,5- a]pyridine-3-carbonitrile | Example 60 | 469.2 | 0.6 |
| 649 | 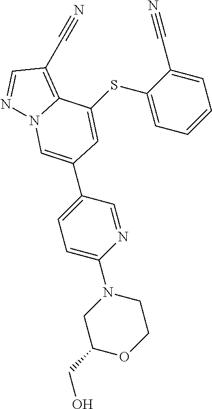 [SMILES: N#Cc1ccccc1Sc1cc(-c2ccc(N3CCO[C@H](CO)C3)nc2)cn2ncc(C#N)c12] | (S)-4-((2- cyanophenyl)thio)-6-(6- (2- (hydroxymethyl)morpholi- no)pyridin-3- yl)pyrazolo[1,5- a]pryidine-3-carbonitrile | Example 60 | 469.2 | 0.5 |
| 650 | 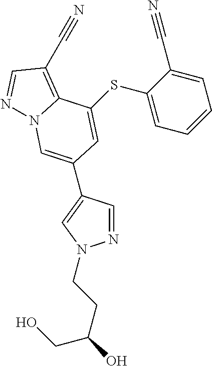 [SMILES: N#Cc1ccccc1Sc1cc(-c2cnn(CC[C@@H](O)CO)c2)cn2ncc(C#N)c12] | (R)-4-((2- cyanophenyl)thio)-6-(1- (3,4-dihydroxybutyl)-1H- pyrazol-4-yl)pyrazolo[1,5- a]pyridine-3-carbonitrile | Example 35 | 431.1 | 1 |
| 651 | 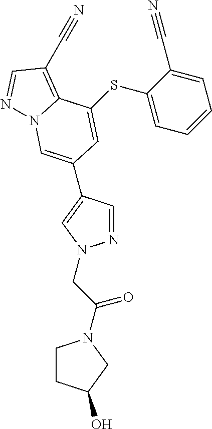 [SMILES: N#Cc1ccccc1Sc1cc(-c2cnn(CC(=O)N3CC[C@H](O)C3)c2)cn2ncc(C#N)c12] | (S)-4-((2- cyanophenyl)thio)-6-(1- (2-(3-hydroxypyrrolidin- 1-yl)-2-oxoethyl)-1H- pyrazol-4-yl)pyrazolo[1,5- a]pyridine-3-carbonitrile | Example 35 | 470.1 | 1 |
| 652 | 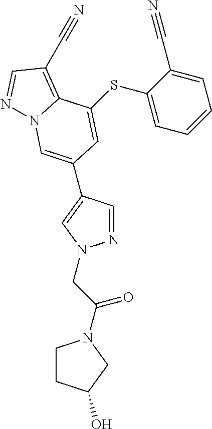 [SMILES: N#Cc1ccccc1Sc1cc(-c2cnn(CC(=O)N3CC[C@@H](O)C3)c2)cn2ncc(C#N)c12] | (R)-4-((2- cyanophenyl)thio)-6-(1- (2-(3-hydroxypyrrolidin- 1-yl)-2-oxoethyl)-1H- pyrazol-4-yl)pyrazolo[1,5- a]pyridine-3-carbonitrile | Example 35 | 470.1 | 1 |
| 653 | 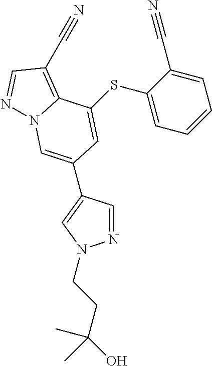 [SMILES: CC(C)(O)CCn1cc(-c2cc(Sc3ccccc3C#N)c3c(C#N)cnn3c2)cn1] | 4-((2-cyanophenyl)thio)- 6-(1-(3-hydroxy-3- methylbutyl)-1H-pyrazol- 4-yl)pyrazolo[1,5- a]pyridine-3-carbonitrile | Example 35 | 429.1 | 1 |
| 654 | 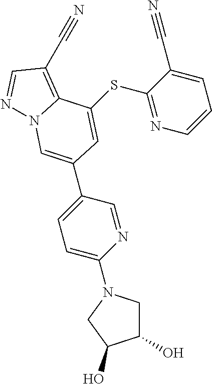 [SMILES: N#Cc1cccnc1Sc1cc(-c2ccc(N3C[C@H](O)[C@@H](O)C3)nc2)cn2ncc(C#N)c12] | 6-(6-((3S,4S)-3,4- dihydroxypyrrolidin-1- yl)pyridin-3-yl)-4-((3- fluoropyridin-2- yl)thio)pyrazolo[1,5- a]pyridine-3-carbonitrile | Example 60 | 449.1 | 3 |
| 655 | 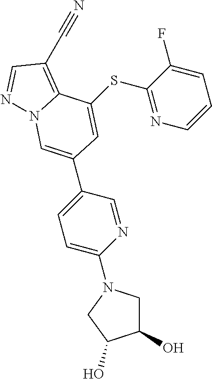 [SMILES: N#Cc1cnn2cc(-c3ccc(N4C[C@@H](O)[C@H](O)C4)nc3)cc(Sc3ncccc3F)c12] | 6-(6-((3R,4R)-3,4- dihydroxypyrrolidin-1- yl)pyridin-3-yl)-4-((3- fluoropyridin-2- yl)thio)pyrazolo[1,5- a]pyridine-3-carbonitrile | Example 60 | 449.1 | 3 |
| 656 | 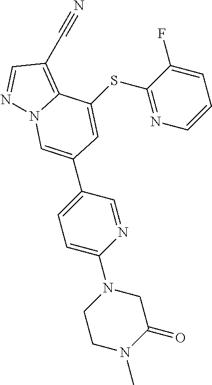 [SMILES: CN1CCN(c2ccc(-c3cc(Sc4ncccc4F)c4c(C#N)cnn4c3)cn2)CC1=O] | 4-((3-fluoropyridin-2- yl)thio)-6-(6-(4-methyl-3- oxopiperazin-1-yl)pyridin- 3-yl)pyrazolo[1,5- a]pyridine-3-carbonitrile | Example 60 | 460.1 | 2 |
| 657 | 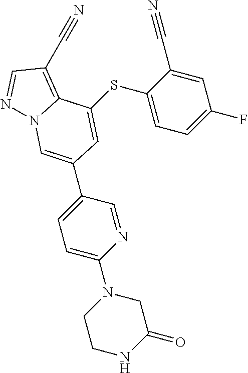 [SMILES: N#Cc1cc(F)ccc1Sc1cc(-c2ccc(N3CCNC(=O)C3)nc2)cn2ncc(C#N)c12] | 4-((2-cyano-4- fluorophenyl)thio)-6-(6- (3-oxopiperazin-1- yl)pyridin-3- yl)pyrazolo[1,5- a]pyridine-3-carbonitrile | Example 60 | 470.1 | 3 |
| 658 | 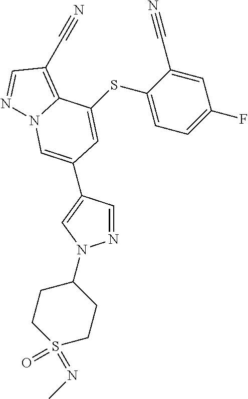 [SMILES: CN=S1(=O)CCC(n2cc(-c3cc(Sc4ccc(F)cc4C#N)c4c(C#N)cnn4c3)cn2)CC1] | 4-((2-cyano-4- fluorophenyl)thio)-6-(1- (1-(methylimino)-1- oxidohexahydro-1l6- thiopyran-4-yl)-1H- pyrazol-4-yl)pyrazolo[1,5- a]pyridine-3-carbonitrile | Example 70 | 506.2 | 1.5 |
| 659 | 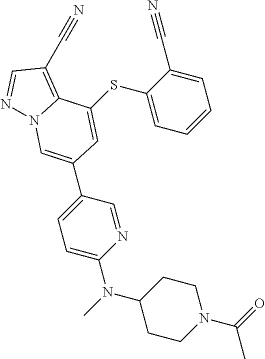 [SMILES: CC(=O)N1CCC(N(C)c2ccc(-c3cc(Sc4ccccc4C#N)c4c(C#N)cnn4c3)cn2)CC1] | 6-(6-((1-acetylpiperidin-4- yl)(methyl)amino)pyridin- 3-yl)-4-((2- cyanophenyl)thio)pyrazolo [1,5-a]pyridine-3- carbonitrile | Example 60 | 508.2 | 0.3 |
| 660 | 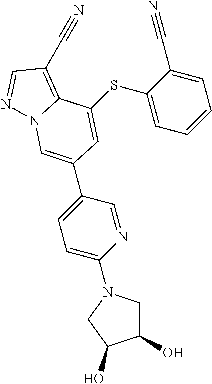 [SMILES: N#Cc1ccccc1Sc1cc(-c2ccc(N3C[C@@H](O)[C@@H](O)C3)nc2)cn2ncc(C#N)c12] | 4-((2-cyanophenyl)thio)- 6-(6-((3R,4S)-3,4- dihydroxypyrrolidin-1- yl)pyridin-3- yl)pyrazolo[1,5- a]pyridine-3-carbonitrile | Example 60 | 455.1 | 1 |
| 661 | 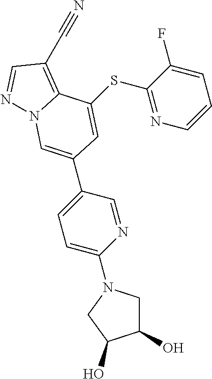 [SMILES: N#Cc1cnn2cc(-c3ccc(N4C[C@@H](O)[C@@H](O)C4)nc3)cc(Sc3ncccc3F)c12] | 6-(6-((3R,4S)-3,4- dihydroxypyrrolidin-1- yl)pyridin-3-yl)-4-((3- fluoropyridin-2- yl)thio)pyrazolo[1,5- a]pyridine-3-carbonitrile | Example 60 | 449.1 | 2 |
| 662 | 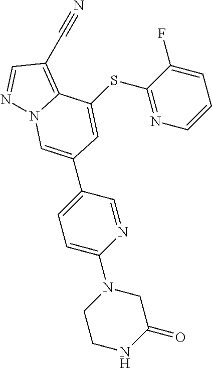 [SMILES: N#Cc1cnn2cc(-c3ccc(N4CCNC(=O)C4)nc3)cc(Sc3ncccc3F)c12] | 4-((3-fluoropyridin-2- yl)thio)-6-(6-(3- oxopiperazin-1-yl)pyridin- 3-yl)pyrazolo[1,5- a]pyridine-3-carbonitrile | Example 60 | 464.2 | 2 |
| 663 | 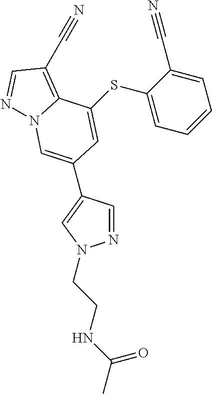 [SMILES: CC(=O)NCCn1cc(-c2cc(Sc3ccccc3C#N)c3c(C#N)cnn3c2)cn1] | N-(2-(4-(3-cyano-4-((2- cyanophenyl)thio)pyrazolo [1,5-a]pyridin-6-yl)-1H- pyrazol-1- yl)ethyl)acetamide | Example 35 | 428.1 | 2 |
| 664 | 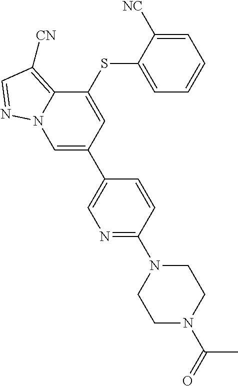 [SMILES: CC(=O)N1CCN(c2ccc(-c3cc(Sc4ccccc4C#N)c4c(C#N)cnn4c3)cn2)CC1] | 6-(6-(4-acetylpiperazin-1- yl)pyridin-3-yl)-4-((2- cyanophenyl)thio)pyrazolo [1,5-a]pyridine-3- carbonitrile | Example 60 | 480.2 | 0.2 |
| 665 | 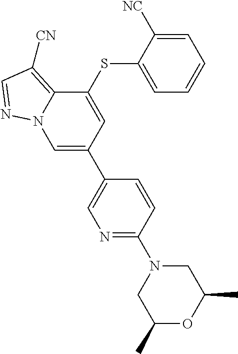 [SMILES: C[C@@H]1CN(c2ccc(-c3cc(Sc4ccccc4C#N)c4c(C#N)cnn4c3)cn2)C[C@H](C)O1] | 4-((2-cyanophenyl)thio)- 6-(6-((2S,6R)-2,6- dimethylmorpholino)pyridin- 3-yl)pyrazolo[1,5- a]pyridine-3-carbonitrile | Example 60 | 467.2 | 1 |
| 666 | 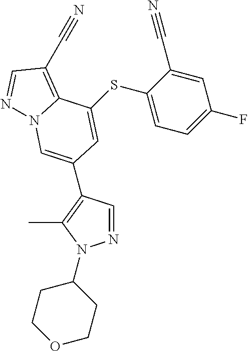 [SMILES: Cc1c(-c2cc(Sc3ccc(F)cc3C#N)c3c(C#N)cnn3c2)cnn1C1CCOCC1] | 4-((2-cyano-4- fluorophenyl)thio)-6-(5- methyl-1-(tetrahydro-2H- pyran-4-yl)-1H-pyrazol-4- y)pyrazolo[1,5- a]pyridine-3-carbonitrile | Example 53 | 459.2 | 0.6 |
| 667 | 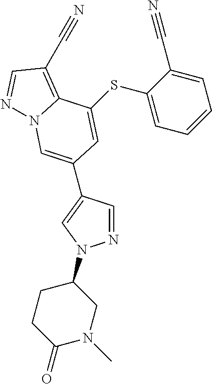 [SMILES: CN1C[C@H](n2cc(-c3cc(Sc4ccccc4C#N)c4c(C#N)cnn4c3)cn2)CCC1=O] | (R)-4-((2- cyanophenyl)thio)-6-(1- (1-methyl-6-oxopiperidin- 3-yl)-1H-pyrazol-4- yl)pyrazolo[1,5- a]pyridine-3-carbonitrile | Example 53 | 454.1 | 1 |
| 668 | 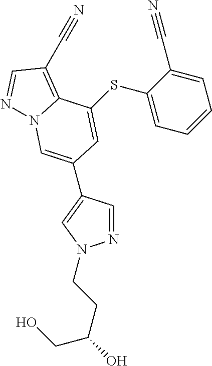 [SMILES: N#Cc1ccccc1Sc1cc(-c2cnn(CC[C@H](O)CO)c2)cn2ncc(C#N)c12] | (S)-4-((2- cyanophenyl)thio)-6-(1- (3,4-dihydroxybutyl)-1H- pyrazol-4-yl)pyrazolo[1,5- a]pyridine-3-carbonitrile | Example 35 | 431.1 | 0.4 |
| 669 | 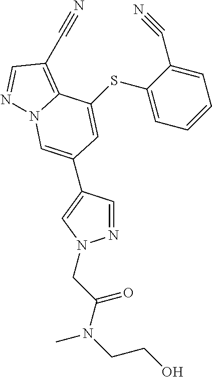 [SMILES: CN(CCO)C(=O)Cn1cc(-c2cc(Sc3ccccc3C#N)c3c(C#N)cnn3c2)cn1] | 2-(4-(3-cyano-4-((2- cyanophenyl)thio)pyrazolo [1,5-a]pyridin-6-yl)-1H- pyrazol-1-yl)-N-(2- hydroxyethyl)-N- methylacetamide | Example 35 | 458.1 | 1 |
| 670 | 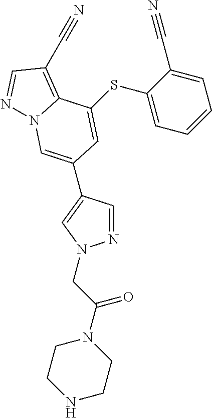 [SMILES: N#Cc1ccccc1Sc1cc(-c2cnn(CC(=O)N3CCNCC3)c2)cn2ncc(C#N)c12] | 4-((2-cyanophenyl)thio)- 6-(1-(2-oxo-2-(piperazin- 1-yl)ethyl)-1H-pyrazol-4- yl)pyrazolo[1,5- a]pryridine-3-carbonitrile | Example 35 | 469.1 | 1 |
| 671 | 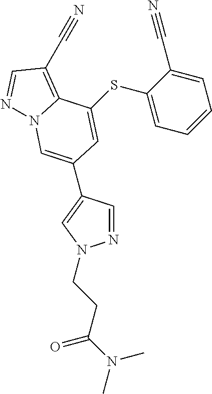 [SMILES: CN(C)C(=O)CCn1cc(-c2cc(Sc3ccccc3C#N)c3c(C#N)cnn3c2)cn1] | 3-(4-(3-cyano-4-((2- cyanophenyl)thio)pyrazolo [1,5-a]pyridin-6-yl)-1H- pyrazol-1-yl)-N,N- dimethylpropanamide | Example 35 | 442.0 | 0.5 |
| 672 | 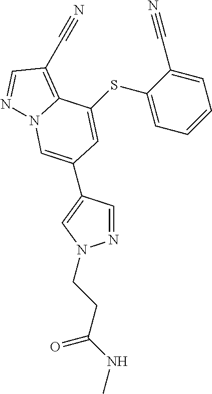 [SMILES: CNC(=O)CCn1cc(-c2cc(Sc3ccccc3C#N)c3c(C#N)cnn3c2)cn1] | 3-(4-(3-cyano-4-((2- cyanophenyl)thio)pyrazolo [1,5-a]pyridin-6-yl)-1H- pyazol-1-yl)-N- methylpropanamide | Example 35 | 428.2 | 1 |
| 673 | 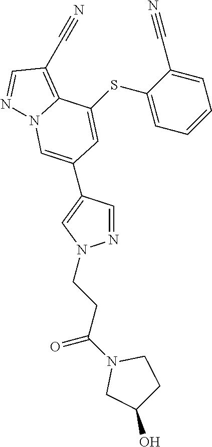 [SMILES: N#Cc1ccccc1Sc1cc(-c2cnn(CCC(=O)N3CC[C@@H](O)C3)c2)cn2ncc(C#N)c12] | (R)-4-((2- cyanophenyl)thio)-6-(1- (3-(3-hydroxypyrrolidin- 1-yl)-3-oxopropyl)-1H- pyrazol-4-yl)pyrazolo[1,5- a]pyridine-3-carbonitrile | Example 35 | 484.1 | 0.2 |
| 674 | 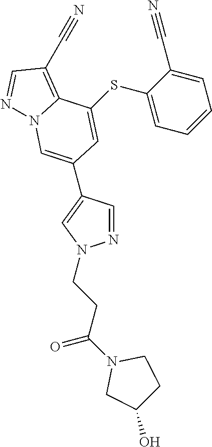 [SMILES: N#Cc1ccccc1Sc1cc(-c2cnn(CCC(=O)N3CC[C@H](O)C3)c2)cn2ncc(C#N)c12] | (S)-4-((2- cyanophenyl)thio)-6-(1- (3-(3-hydroxypyrrolidin- 1-yl)-3-oxopropyl)-1H- pyrazol-4-yl)pyrazolo[1,5- a]pyridine-3-carbonitrile | Example 35 | 484.2 | 1 |
| 675 | 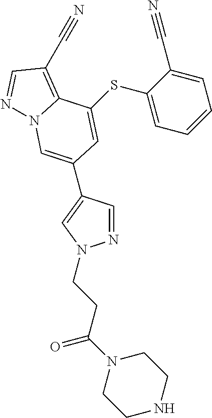 [SMILES: N#Cc1ccccc1Sc1cc(-c2cnn(CCC(=O)N3CCNCC3)c2)cn2ncc(C#N)c12] | 4-((2-cyanophenyl)thio)- 6-(1-(3-oxo-3-(piperazin- 1-yl)propyl)-1H-pyrazol- 4-yl)pyrazolo[1,5- a]pyridine-3-carbonitrile | Example 35 | 483.0 | 0.6 |
| 676 | 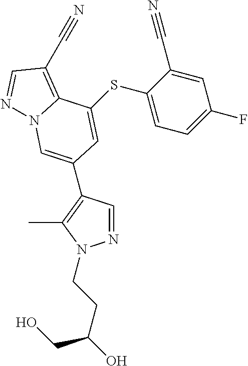 [SMILES: Cc1c(-c2cc(Sc3ccc(F)cc3C#N)c3c(C#N)cnn3c2)cnn1CC[C@@H](O)CO] | (R)-4-((2-cyano-4- fluorophenyl)thio)-6-(1- (3,4-dihydroxybutyl)-5- methyl-1H-pyrazol-4- yl)pyrazolo[1,5- a]pyridine-3-carbonitrile | Example 35 | 463.0 | 3 |
| 677 | 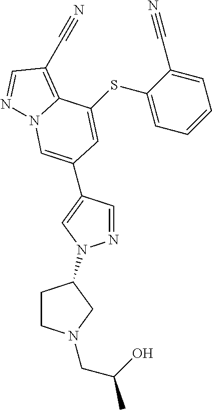 [SMILES: C[C@H](O)CN1CC[C@H](n2cc(-c3cc(Sc4ccccc4C#N)c4c(C#N)cnn4c3)cn2)C1] | 4-((2-cyanophenyl)thio)- 6-(1-((S)-1-((S)-2- hydroxypropyl)pyrrolidin- 3-yl)-1H-pyrazol-4- y)pyrazolo[1,5- a]pyridine-3-carbonitrile | Example 35 | 470.2 | 0.3 |
| 678 | 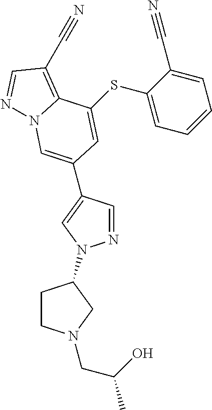 [SMILES: C[C@@H](O)CN1CC[C@H](n2cc(-c3cc(Sc4ccccc4C#N)c4c(C#N)cnn4c3)cn2)C1] | 4-((2-cyanophenyl)thio)- 6-(1-((S)-1-((R)-2- hydroxypropyl)pyrrolidin- 3-yl)-1H-pyrazol-4- yl)pyrazolo[1,5- a]pyridine-3-carbonitrile | Example 35 | 469.9 | 0.4 |
| 679 | 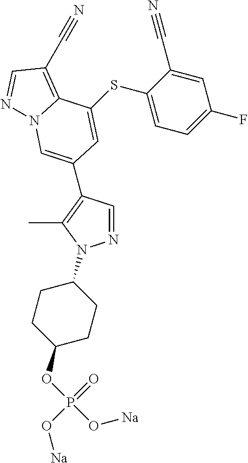 | sodium (1r,4r)-4-(4-(3- cyano-4-((2-cyano-4- fluorophenyl)thio)pyrazolo [1,5-a]pyridin-6-yl)-5- methyl-1H-pyrazol-1- yl)cyclohexyl phosphate | Example 71 | 553.0 | 1.6 |
| 680 | 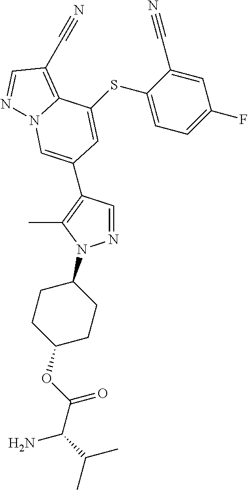 [SMILES: Cc1c(-c2cc(Sc3ccc(F)cc3C#N)c3c(C#N)cnn3c2)cnn1[C@H]1CC[C@H](OC(=O)[C@@H](N)C(C)C)CC1] | (1r,4S)-4-(4-(3-cyano-4- ((2-cyano-4- fluorophenyl)thio)pyrazolo [1,5-a]pyridin-6-yl)-5- methyl-1H-pyrazol-1- yl)cyclohexyl L-valinate | Example 36 | 572.4 | 1 |

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

1. A compound having the following structure of Formula (I): 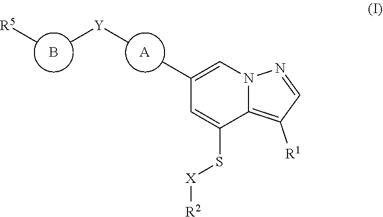
wherein:
R¹ is —CN, —Cl, —Br, —CF₂H, or —CF₃;
R² is C₁-C₆ alkyl, C₁-C₆ heteroalkyl, aryl, heteroaryl, a fused bicyclic, C₃-C₈ cycloalkyl, C₃-C₈ heterocycloalkyl, or —NR³R⁴;
R³ and R⁴ are, each independently, H or C₁-C₄ alkyl;
R⁵ is —H, —OH, —CN, —F, C₁-C₆ alkyl, C₃-C₈ cycloalkyl, C₁-C₆ heteroalkyl, a 5-6 membered heteroaryl, C₃-C₈ heterocycloalkyl, —C(═O)R⁶, —OC(═O)R⁶, —CH₂C(═O)OR⁶, —NR⁷R⁸, —NR⁷C(═O)R⁶, —NR⁷C(═O)OR⁶, —CH₂C(═O)NR⁷N⁸, —NR⁷R⁸CH₂C(═O)NR⁷N⁸, —CH₂NR⁷C(═O)R⁶, sulfonylmethane, oxo, phosphate, or a combination thereof;
wherein the C₁-C₆ alkyl or C₁-C₆ heteroalkyl of R⁵ is optionally substituted with 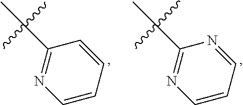 [SMILES: *c1ccccn1 ; *c1ncccn1]
or —OH;
R⁶ is a hydrogen, C₁-C₄ alkyl, C₁-C₅ heteroalkyl, or C₄-C₆ heterocycloalkyl, wherein the C₄-C₆ heterocycloalkyl is substituted with 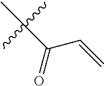 [SMILES: *C(=O)C=C]
or halo;
R⁷ and R⁸ are, each independently, H, C₁-C₆ alkyl, C₁-C₆ alkyl alcohol, sulfonylmethane, C₃-C₆ cycloalkyl, or 5-6 membered heteroaryl, wherein the C₁-C₆ alkyl or C₃-C₆ cycloalkyl is optionally substituted with fluoro, C₃-C₆ cycloalkyl, or 5-6 membered heteroaryl;
X is —(CH₂)n-, —(CH₂)nO—, —(CHCH₃)n-, —(CHCH₃)n-(CH₂)m-, —(CHCH₃)n-(CH₂)mO—, or a direct bond;
Y is —CH₂—, —CHCH₃—, —C(CH₃)₂—, —(CH₂)ₚC(═O)—, —NCH₃—, or a direct bond;
n is an integer between 1 and 4;
m is an integer between 1 and 4;
p is an integer between 0 and 4;
A is an aryl, a 5-6 membered heteroaryl, a halo, or a fused heterocyclic; and
B is C₃-C₈ cycloalkyl, C₃-C₈ heterocycloalkyl, a fused bicyclic, a bridged bicyclic, a spirocyclic, or absent,
or a stereoisomer of the compound, salt or tautomer of the compound thereof.

2. The compound of claim 1, wherein R¹ has one of the following structures:

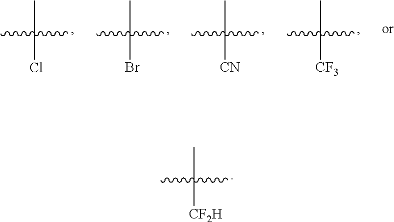

3. The compound of any one of claims 1-2, wherein R¹ is —CN.

4. The compound of claim 3, wherein the compound has the following structure of Formula (IA): 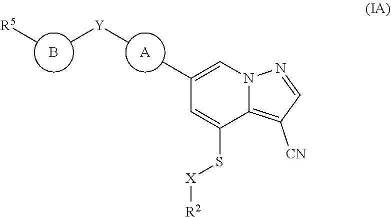
or a stereoisomer of the compound, salt or tautomer of the compound thereof.

5. The compound of any one of claims 1-4, wherein n is an integer of 1 or 2.

6. The compound of any one of claims 1-5, wherein m is an integer of 1 or 2.

7. The compound of any one of claims 1-6, wherein X is a direct bond.

8. The compound of claim 7, wherein the compound has the following structure of Formula (IB): 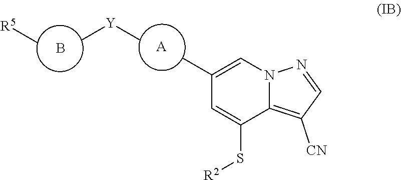
or a stereoisomer of the compound, salt or tautomer of the compound thereof.

9. The compound of any one of claims 1-8, wherein the aryl of A is a phenyl.

10. The compound of any one of claims 1-9, wherein the 5-6 membered heteroaryl of A is a 5 membered heteroaryl.

11. The compound of any one of claims 1-10, wherein the 5 membered heteroaryl is pyrazole or triazole.

12. The compound of any one of claims 1-10, wherein the 5-6 membered heteroaryl of A is a 6 membered heteroaryl.

13. The compound of any one of claims 1-12, wherein the 6 membered heteroaryl is pyridine.

14. The compound of any one of claims 1-13, wherein the 5-6 membered heteroaryl of A is further substituted with C₁-C₄ alkyl, C₁-C₄ heteroalkyl, or halo.

15. The compound of any one of claims 1-14, wherein the 5-6 membered heteroaryl is 5-methylpyrazole.

16. The compound of any one of claims 1-15, wherein A is —F, —Cl, or —Br.

17. The compound of any one of claims 1-16, wherein the fused heterocyclic of A is indole, isoindole, benzimidazole, purine, indazole, benzoxazole, benzisooxazole, quinoline, isoquinoline, quinoxaline, quinazoline, cinnoline, phtalazine, 5H-pyrrolo[2,3-b]pyrazine, or 4,5,6,7-tetrahydropyrazolo[1,5-a]pyrazine.

18. The compound of any one of claims 1-17, wherein the fused heterocyclic of A is 5H-pyrrolo[2,3-b]pyrazine or 4,5,6,7-tetrahydropyrazolo[1,5-a]pyrazine.

19. The compound of any one of claims 1-18, wherein the aryl, 5-6 membered heteroaryl, or fused heterocyclic of A is mono or di-substituted with —CH₃, —F, —Cl, —CN, —NH₂, —CH₂OCH₃, or a combination thereof.

20. The compound of any one of claims 1-19, wherein A has one of the following structures: 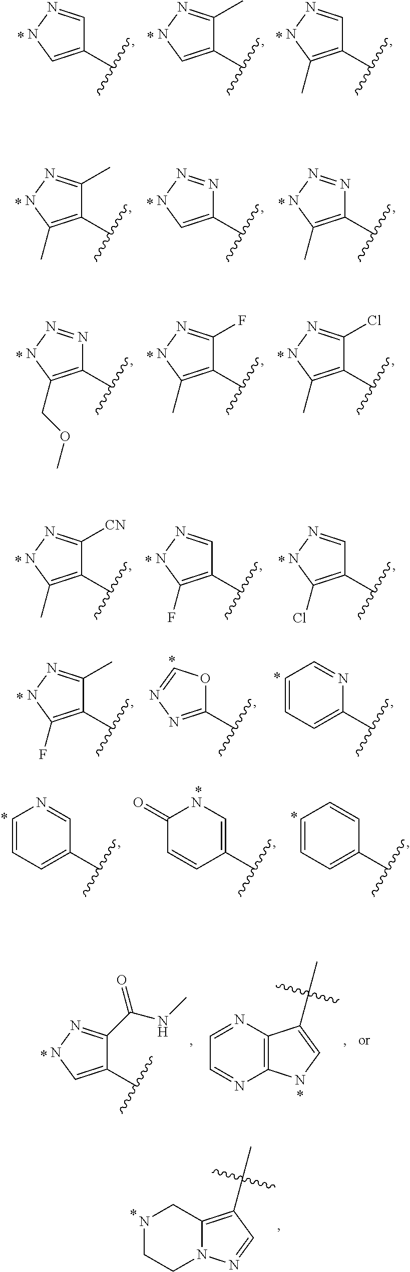
wherein * indicates a location of a bond to Y.

21. The compound of any one of claims 1-20, wherein A has one of the following structures: 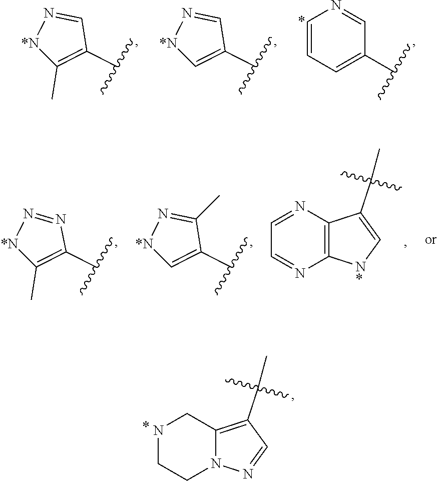
wherein * indicates a location of a bond to Y.

22. The compound of claim 21, wherein A has one of the following structures: 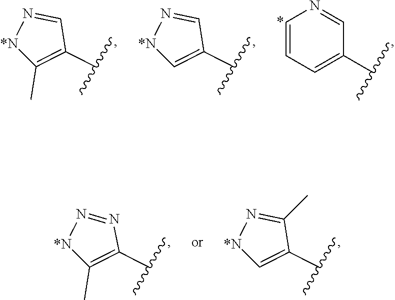
wherein * indicates a location of a bond to Y.

23. The compound of claim 22, wherein the compound has one of the following structures of Formula (IC)-(IG): 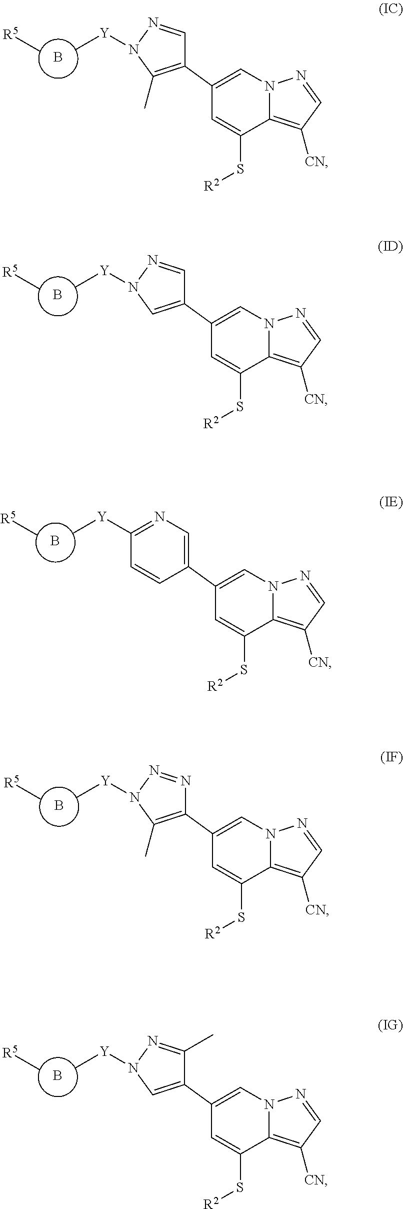
or a stereoisomer of the compound, salt or tautomer of the compound thereof.

24. The compound of any one of claims 1-23, wherein Y is —CH₂—.

25. The compound of any one of claims 1-24, wherein Y is —CHCH₃—.

26. The compound of any one of claims 1-25, wherein Y is C(CH₃)₂—.

27. The compound of any one of claims 1-26, wherein Y is —(CH₂)ₚC(═O)—, wherein p is an integer between 0 and 2.

28. The compound of any one of claims 1-27, wherein Y is —NCH₃—.

29. The compound of any one of claims 1-28, wherein Y is a direct bond.

30. The compound of claim 29, wherein the compound has one of the following structures of Formula (IH)-(IL): 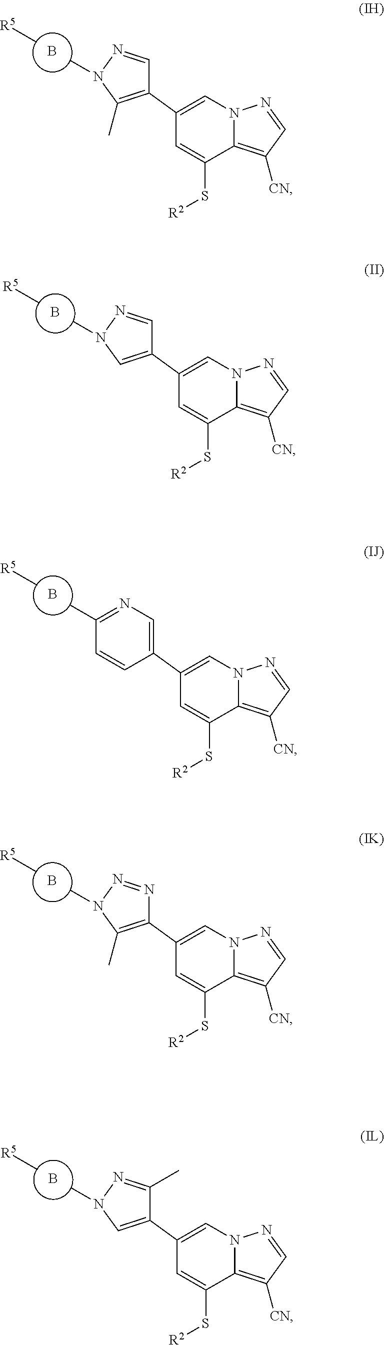
or a stereoisomer of the compound, salt or tautomer of the compound thereof.

31. The compound of any one of claims 1-30, wherein the aryl, heteroaryl, or fused bicyclic of R² is further substituted with fluoro, chloro, cyano, methyl, amine, —OCH₃, —CF₂H, —CF₃, C₁-C₃ alkylalcohol, C₁-C₃ alkoxyl, or a combination thereof.

32. The compound of any one of claims 1-31, wherein the fused bicyclic of R² has one of the following structures:

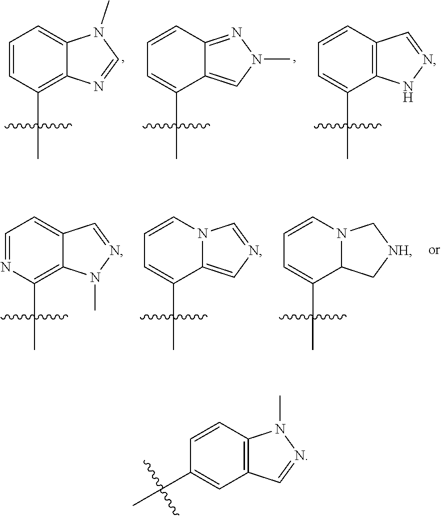

33. The compound of any one of claims 1-32, wherein the fused bicyclic of R² has one of the following structures:

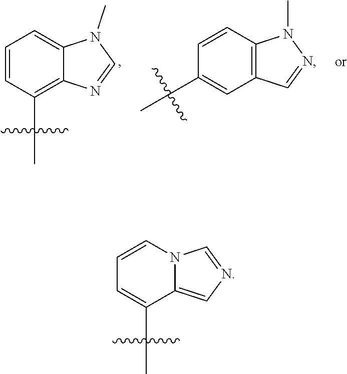 [SMILES: *c1cccc2c1ncn2C ; *c1ccc2c(cnn2C)c1 ; *c1cccn2cncc12]

34. The compound of any one of claims 1-33, wherein the aryl of Re has one of the following structures:

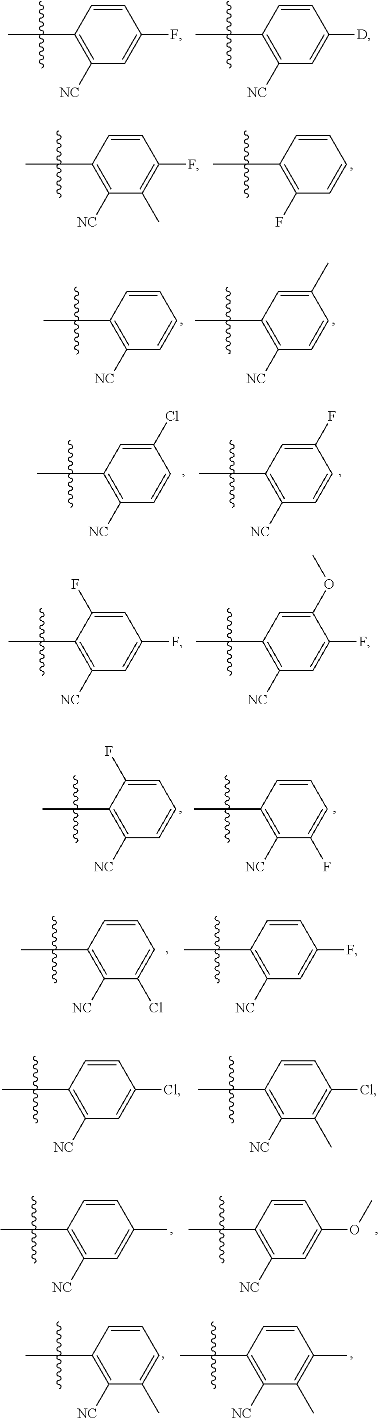

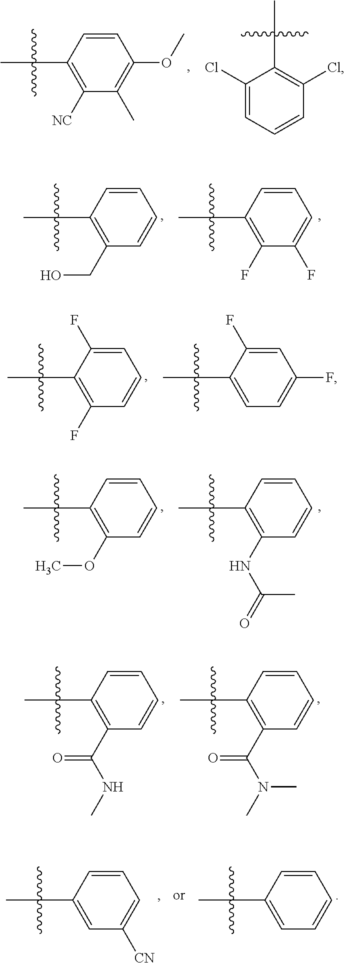

35. The compound of any one of claims 1-34, wherein the heteroaryl of R² has one of the following structures:

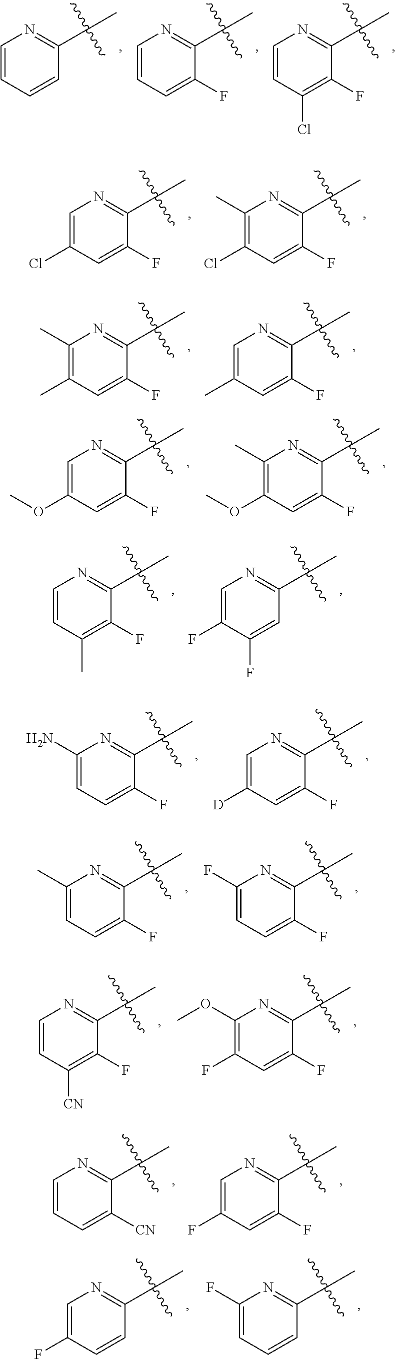

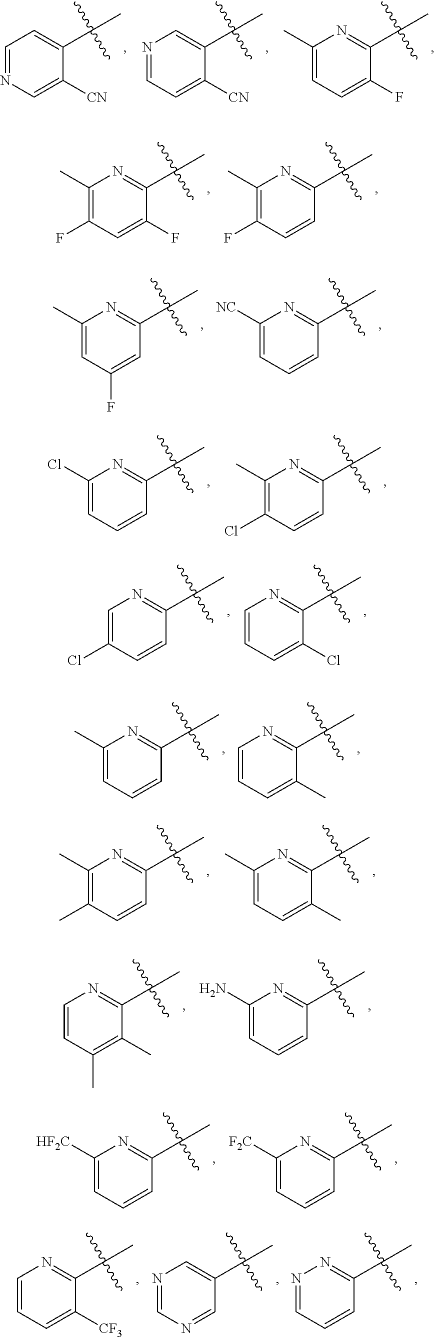

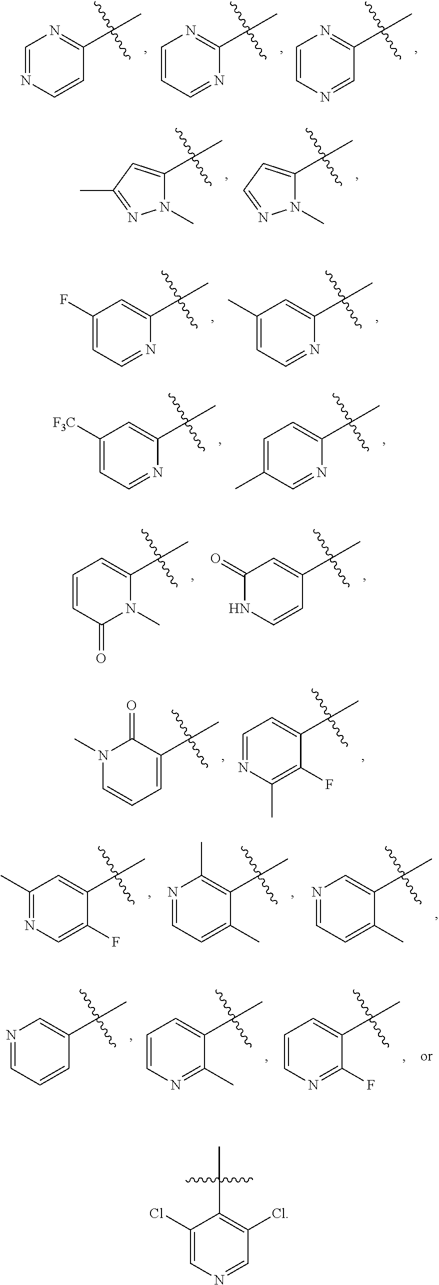

36. The compound of any one of claims 1-35, wherein the C₃-C₈ cycloalkyl or C₃-C₈ heterocycloalkyl of R² has one of the following structures:

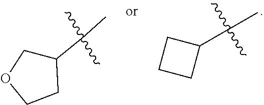 [SMILES: *C1CCOC1 ; *C1CCC1]

37. The compound of any one of claims 1-36, wherein R² has one of the following structures:

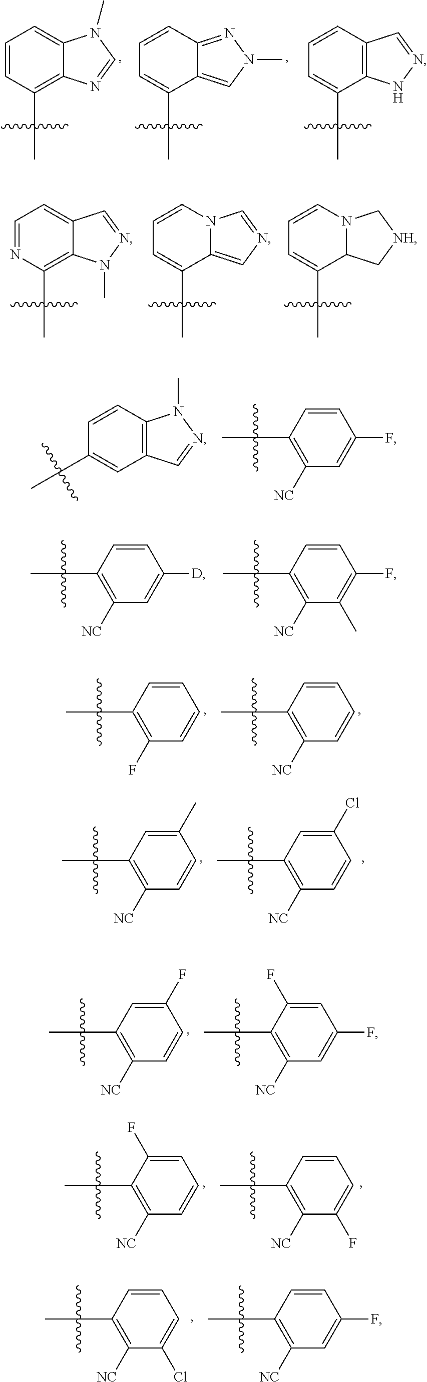

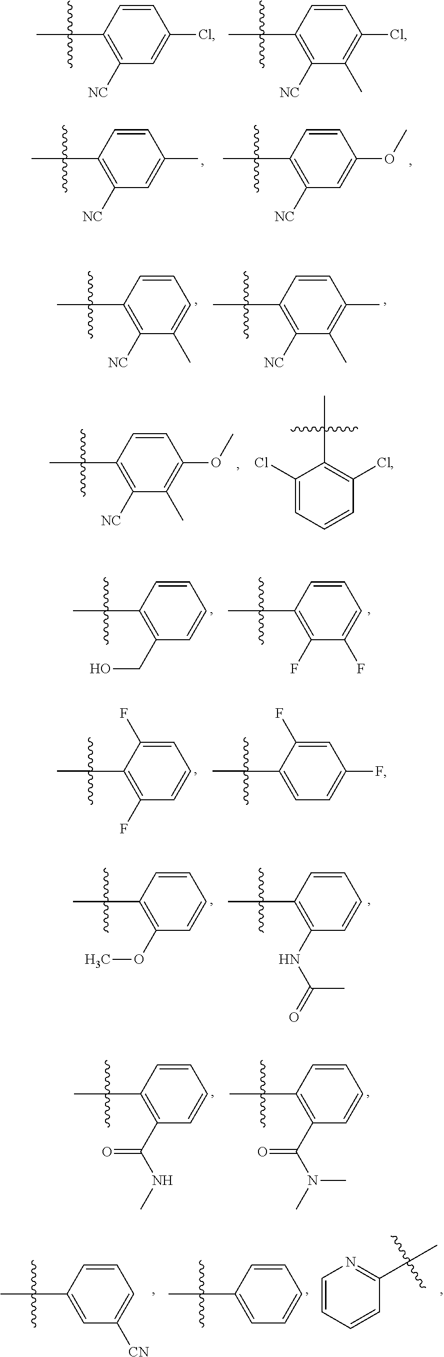

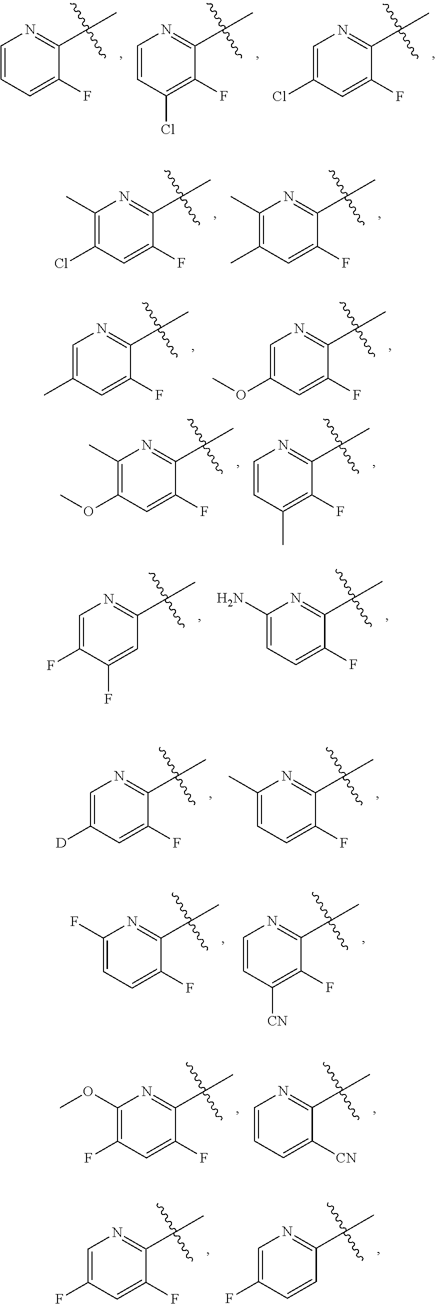

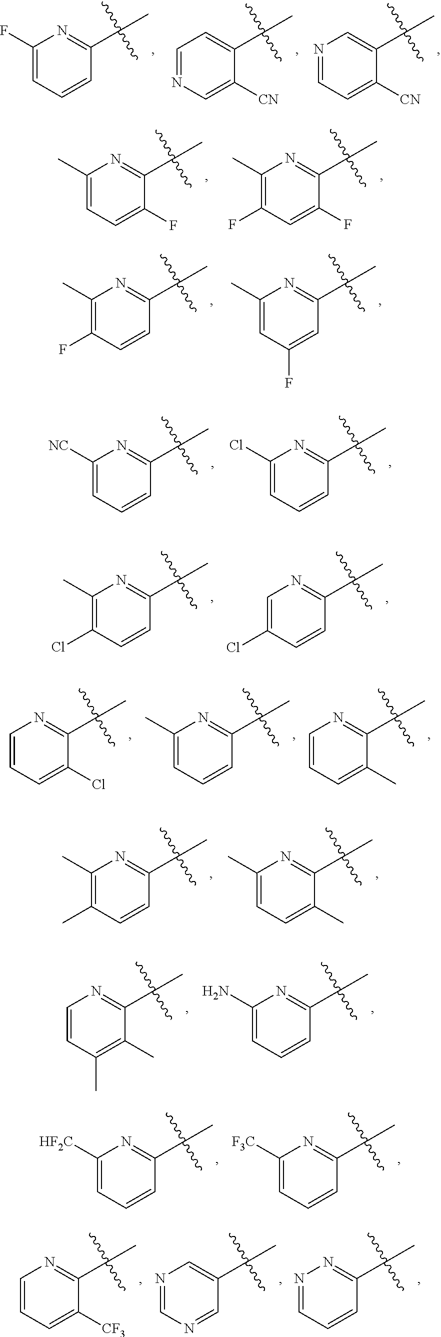

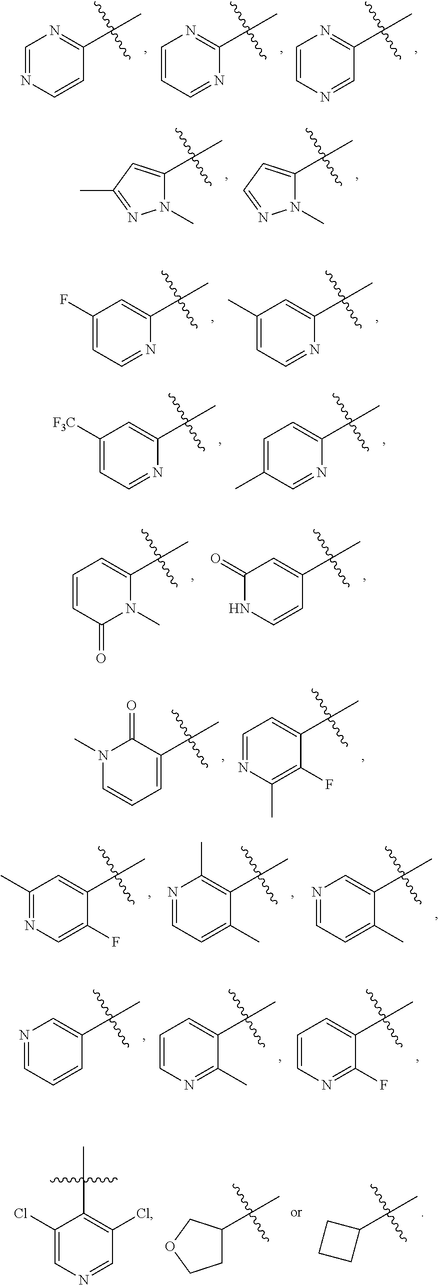

38. The compound of any one of claims 1-37, wherein R³ and R⁴ are, each independently, H or C₁ alkyl.

39. The compound of any one of claims 1-38, wherein the C₃-C₈ cycloalkyl, C₃-C₈ heterocycloalkyl, a fused bicyclic, a bridged bicyclic, or a spirocyclic of B is further substituted with fluoro, —OH, or methyl.

40. The compound of any one of claims 1-39, wherein the C₃-C₈ heterocycloalkyl of B is sulfoximine or sulfone.

41. The compound of any one of claims 1-40, wherein the C₃-C₈ heterocycloalkyl of B is

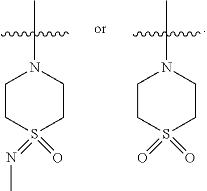

42. The compound of any one of claims 1-41, wherein B is C₆-C₇ cycloalkyl, C₄-C₆ heterocycloalkyl, a fused bicyclic, a bridged bicyclic, or a spirocyclic.

43. The compound of any one of claims 1-42, wherein B is C₆ cycloalkyl, C₄-C₅ heterocycloalkyl, a fused bicyclic, a bridged bicyclic, or a spirocyclic.

44. The compound of any one of claims 1-43, wherein the fused bicyclic of B is octahydrocyclopenta[c]pyrrole, 3-azabicyclo[3.2.0]heptane, or 3-azabicyclo[3.1.0]hexane.

45. The compound of any one of claims 1-44, wherein the bridged bicyclic of B is 3-azabicyclo[3.2.1]octane, 8-azabicyclo[3.2.1]octane, or 2-azabicyclo[2.2.1]heptane.

46. The compound of any one of claims 1-45, wherein the spirocyclic of B is 1-azaspiro[4.5]decane, 2-azaspiro[4.5]decane, 3-azaspiro[5.5]undecane, 2-azaspiro[3.5]nonane, 2-azaspiro[3.3]heptane, 7-azaspiro[3.5]nonane, 1-azaspiro[3.3]heptane, or spiro[3.3]heptane.

47. The compound of any one of claims 1-46, wherein B has one of the following structures: 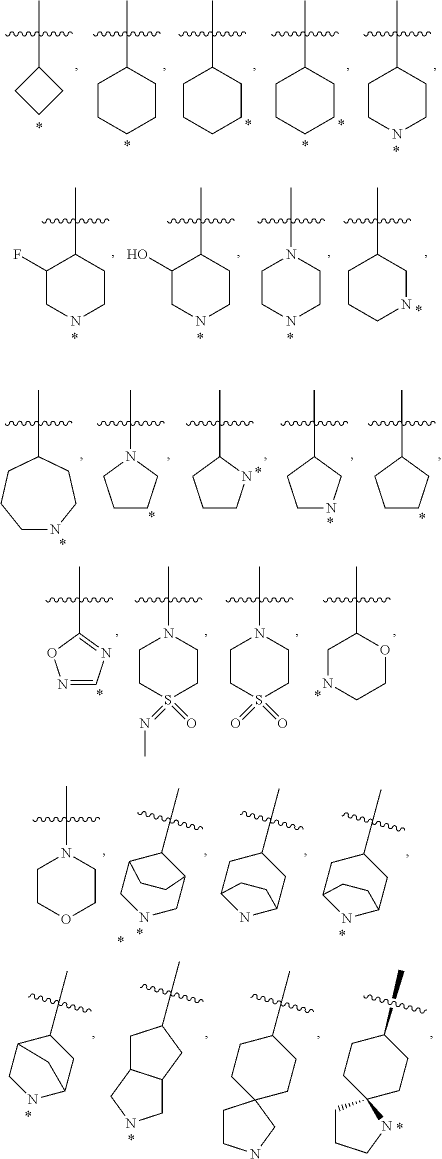 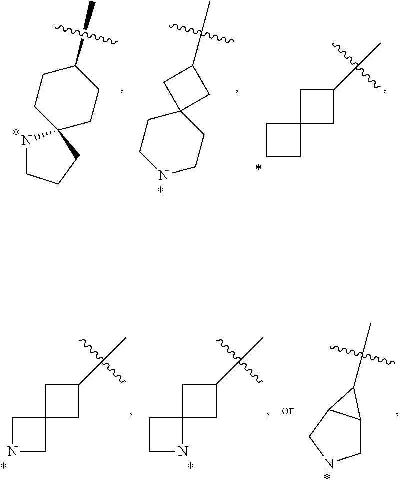
wherein * indicates a location of a bond to R⁵.

48. The compound of any one of claims 1-47, wherein B is substituted with one or more R⁵.

49. The compound of any one of claims 1-48, wherein B is absent.

50. The compound of any one of claims 1-49, wherein R⁵ is —H, —OH, —CN, —F, C₁-C₄ alkyl, C₃-C₆ cycloalkyl, C₁-C₄ heteroalkyl, 6 membered heteroaryl, C₄-C₆ heterocycloalkyl —OC(═O)R⁶, —CH₂C(═O)OR⁶, —NR⁷R⁸, —NR⁷C(═O)R⁶, —NR⁷C(═O)OR⁶, —CH₂C(═O)NR⁷N⁸, —NR⁷R⁸CH₂C(═O)NR⁷N⁸, —CH₂NR⁷C(═O)R⁶, sulfonylmethane, oxo, phosphate, or a combination thereof.

51. The compound of any one of claims 1-50, wherein the C₁-C₆ heteroalkyl of R⁵ is C₁-C₆ deutro-alkyl.

52. The compound of any one of claims 1-51, wherein the C₄-C₆ heterocycloalkyl of R⁶ is pyrrolidine or 1-methyl pyrrolidine.

53. The compound of any one of claims 1-52, wherein R⁶ is C₁-C₃ alkyl, C₁-C₄ heteroalkyl, or C₄-C₅ heterocycloalkyl.

54. The compound of any one of claims 1-53, wherein the C₁-C₄ heteroalkyl of R⁶ is C₁-C₄ fluoroalkyl.

55. The compound of any one of claims 1-54, wherein the C₄-C₆ heterocycloalkyl of R⁶ is substituted with 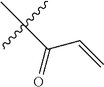 [SMILES: *C(=O)C=C]
or fluoro.

56. The compound of any one of claims 1-55, wherein the C₄-C₆ heterocycloalkyl of R⁶ substituted with

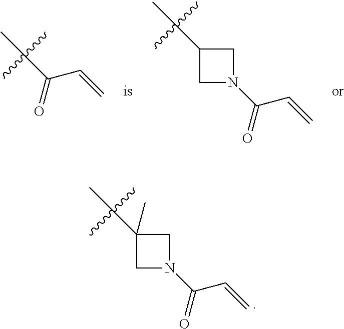

57. The compound of any one of claims 1-56, wherein the C₄-C₆ heterocycloalkyl of R⁶ substituted with fluoro is

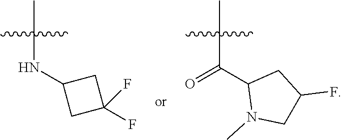

58. The compound of any one of claims 1-57, wherein the 5-6 membered heteroaryl of R⁵ is

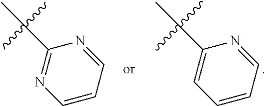 [SMILES: *c1ncccn1 ; *c1ccccn1]

59. The compound of any one of claims 1-58, wherein the C₃-C₈ heterocycloalkyl of R⁵ is

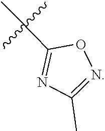 [SMILES: *c1nc(C)no1]

60. The compound of any one of claims 1-59, wherein the C₃-C₈ cycloalkyl of R⁵ is

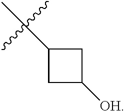 [SMILES: *C1CC(O)C1]

61. The compound of any one of claims 1-60, wherein the oxo of R⁵ is

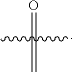 [SMILES: *=O]

62. The compound of any one of claims 1-61, wherein the phosphate of R⁵ is

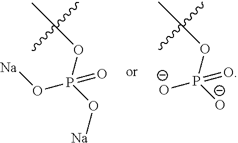

63. The compound of any one of claims 1-62, wherein R⁷ and R⁸ are, each independently, H, C₁-C₃ alkyl, C₁-C₃ alkyl alcohol, sulfonylmethane, C₃-C₄ cycloalkyl, or 6 membered heteroaryl, wherein the C₁-C₃ alkyl or C₃-C₄ cycloalkyl is optionally substituted with fluoro or C₃-C₄ cycloalkyl.

64. The compound of any one of claims 1-63, wherein R⁵ has one of the following structures:

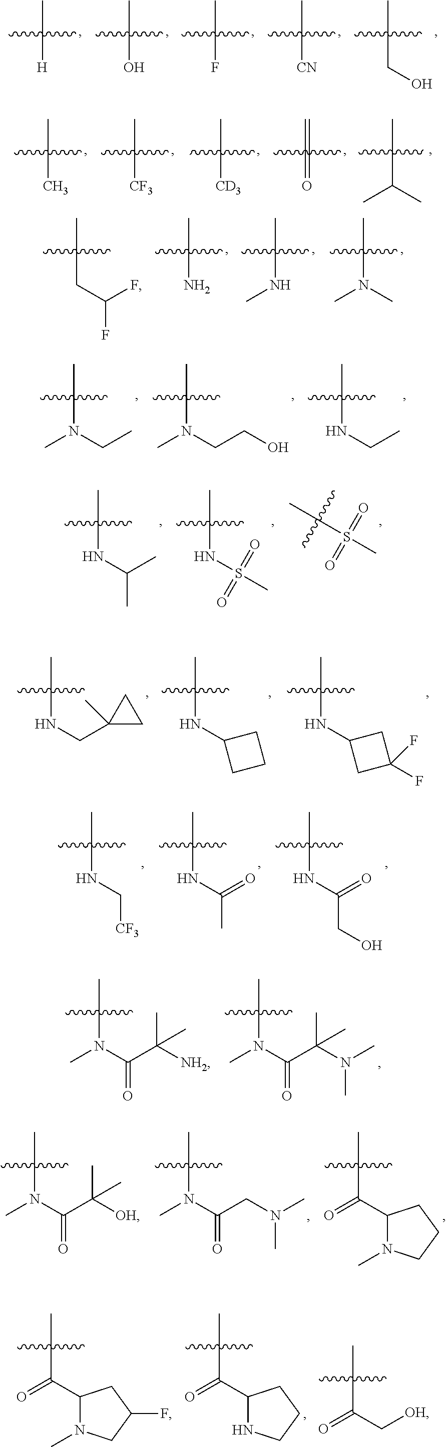

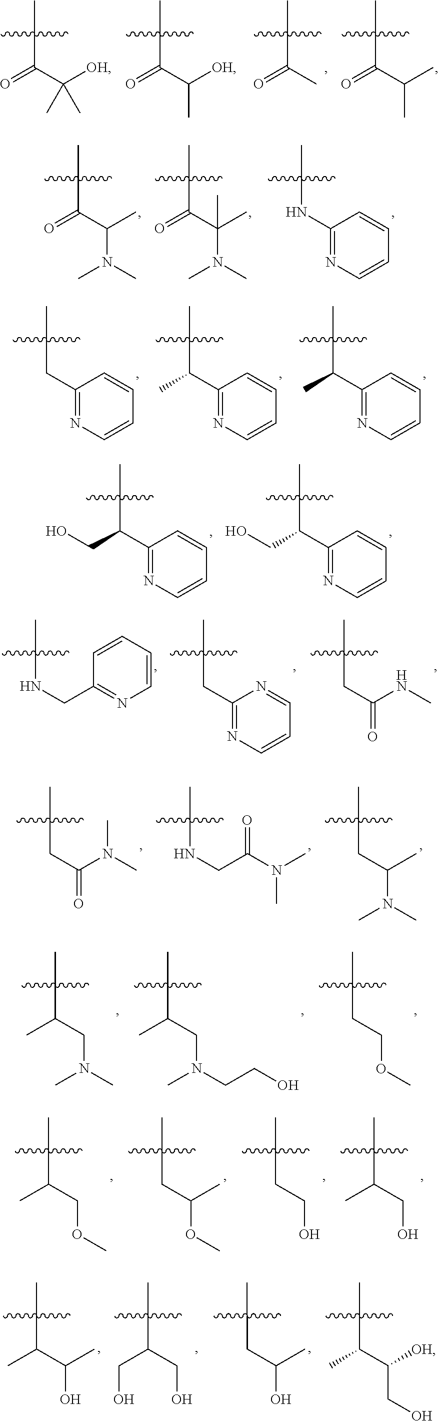

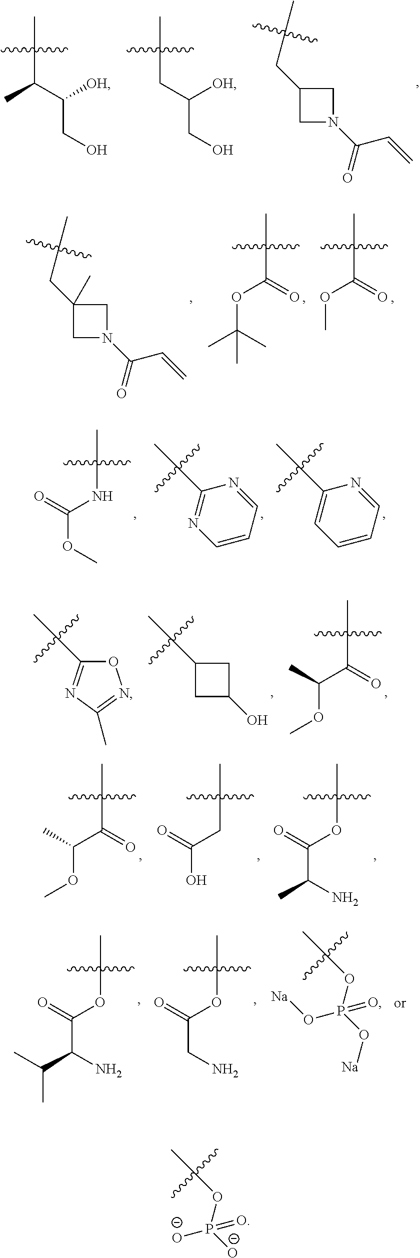

65. The compound of claim 1, wherein the compound has one of the following structures:

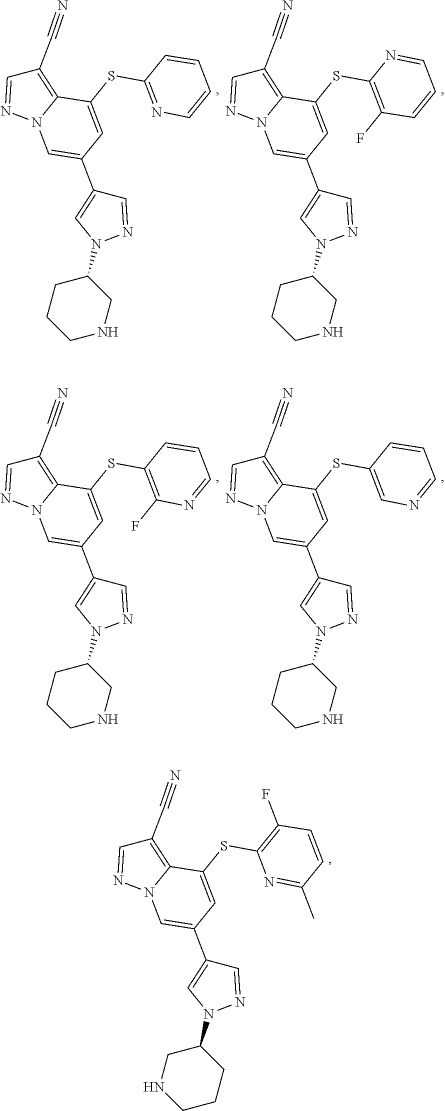

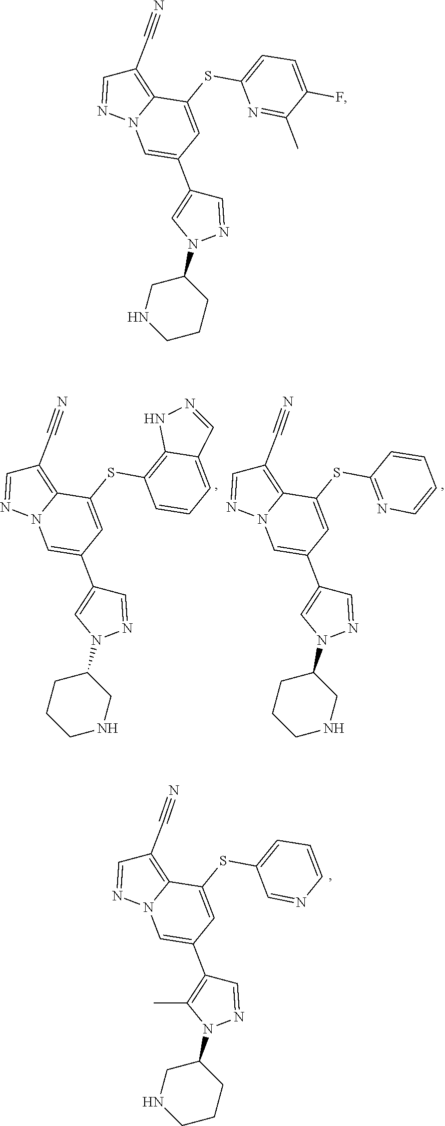

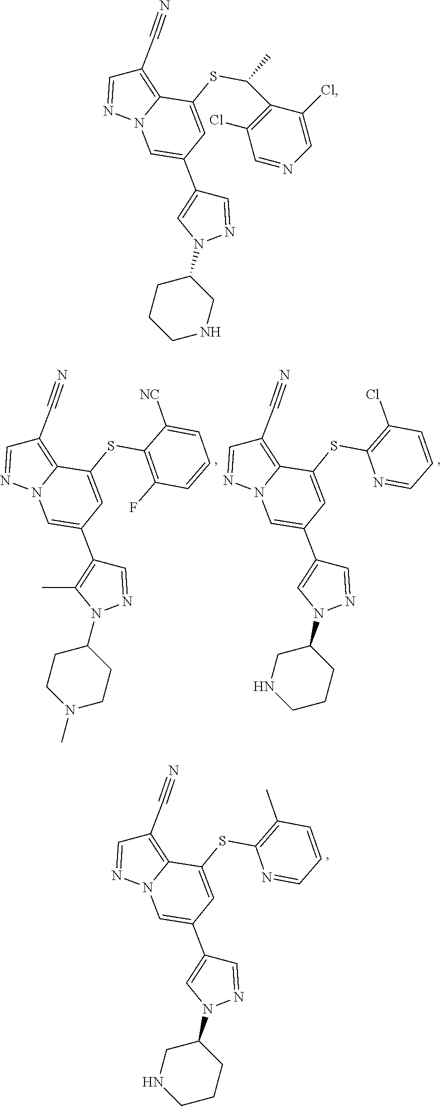

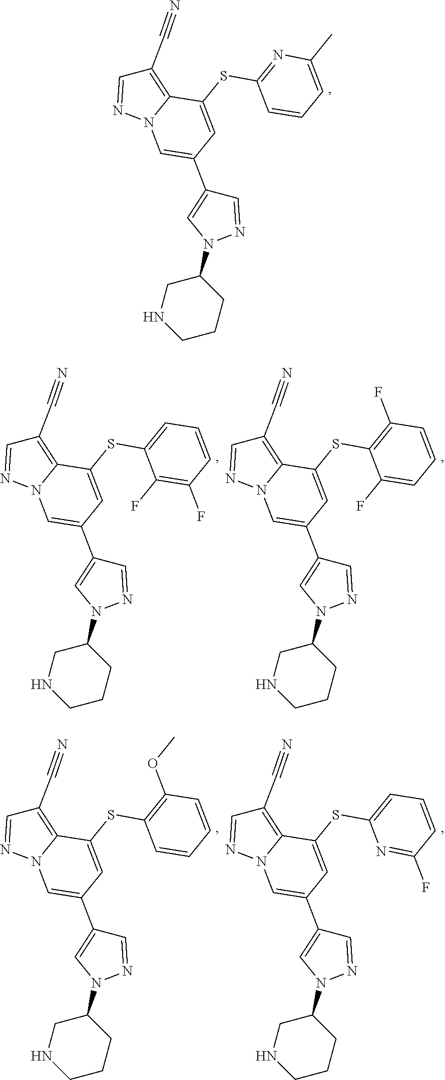

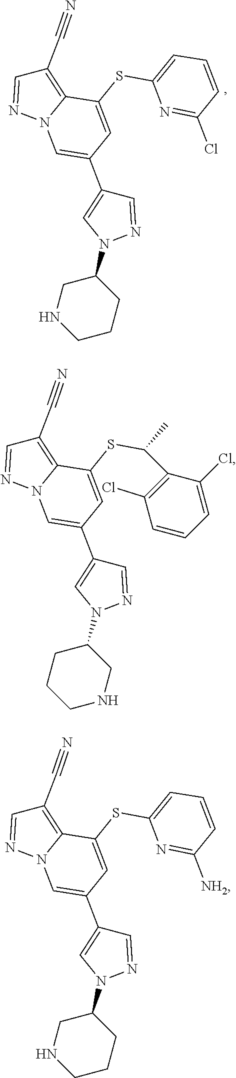

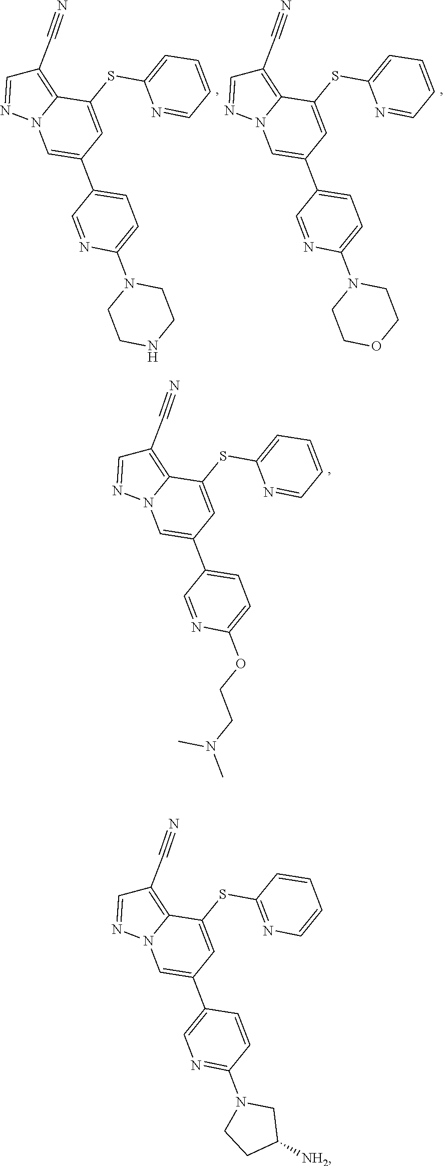

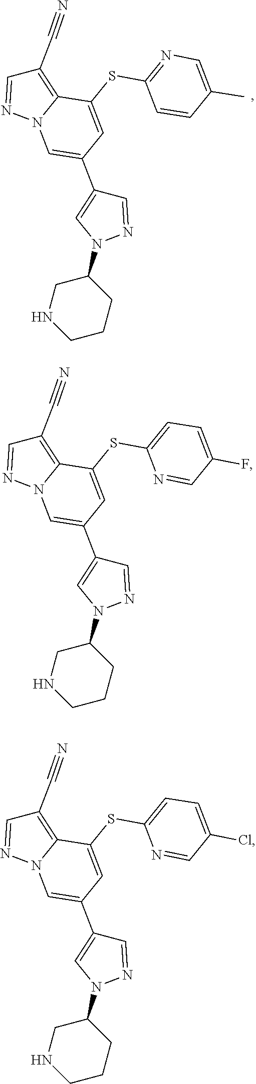

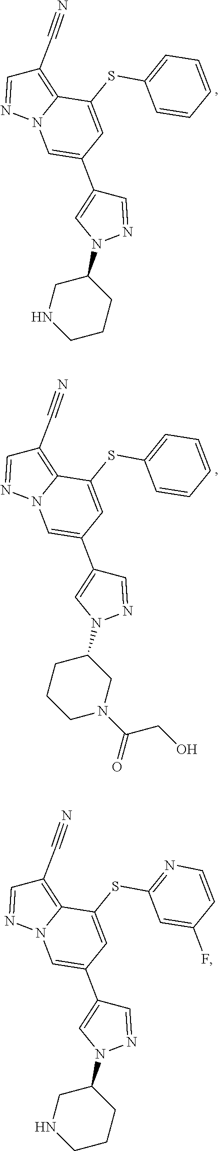

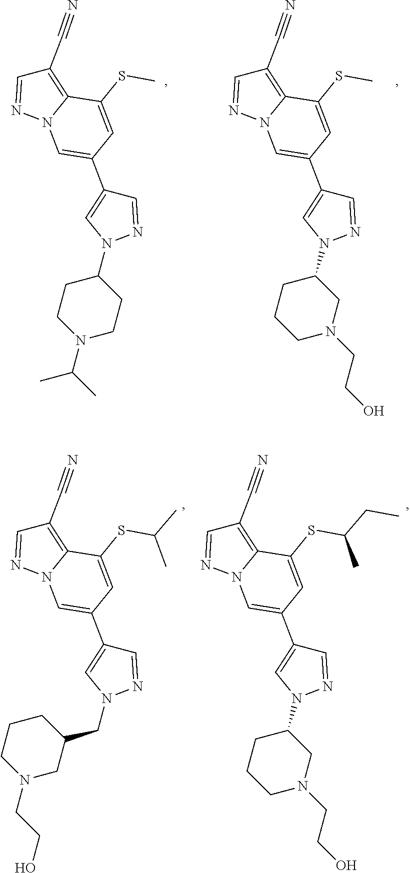

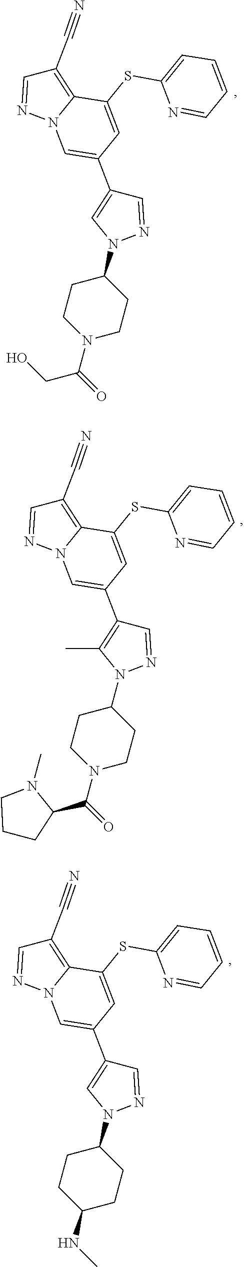

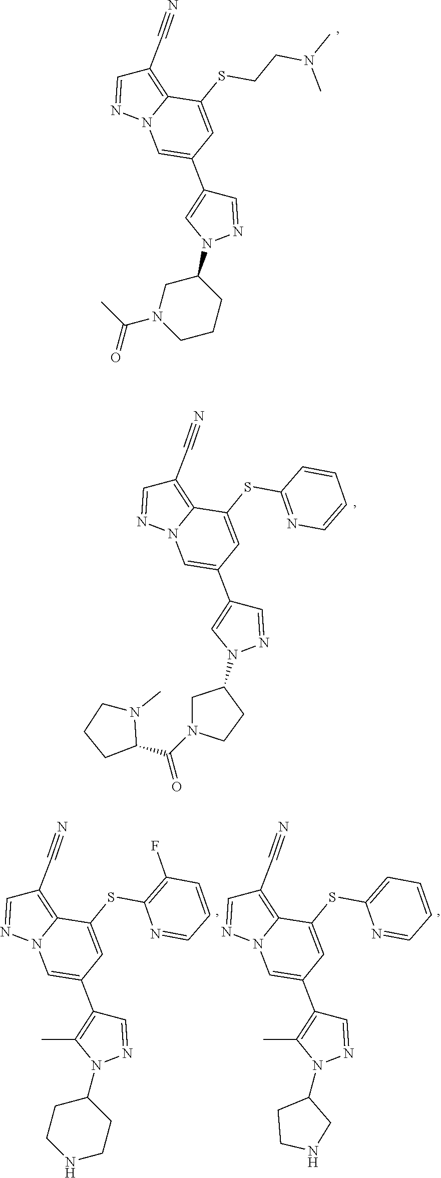

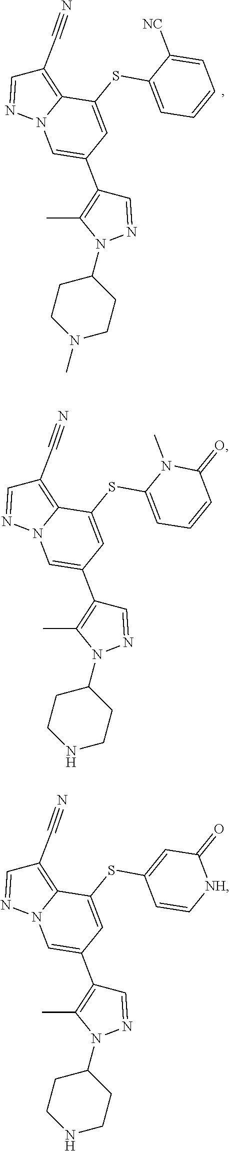

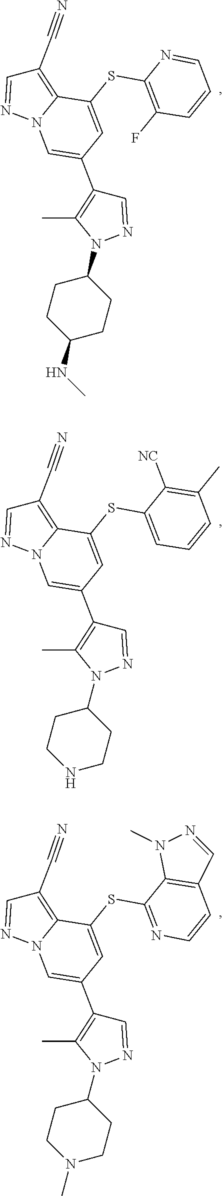 [SMILES: CN[C@H]1CC[C@@H](n2ncc(-c3cc(Sc4ncccc4F)c4c(C#N)cnn4c3)c2C)CC1 ; Cc1cccc(Sc2cc(-c3cnn(C4CCNCC4)c3C)cn3ncc(C#N)c23)c1C#N ; Cc1c(-c2cc(Sc3nccc4cnn(C)c34)c3c(C#N)cnn3c2)cnn1C1CCN(C)CC1]

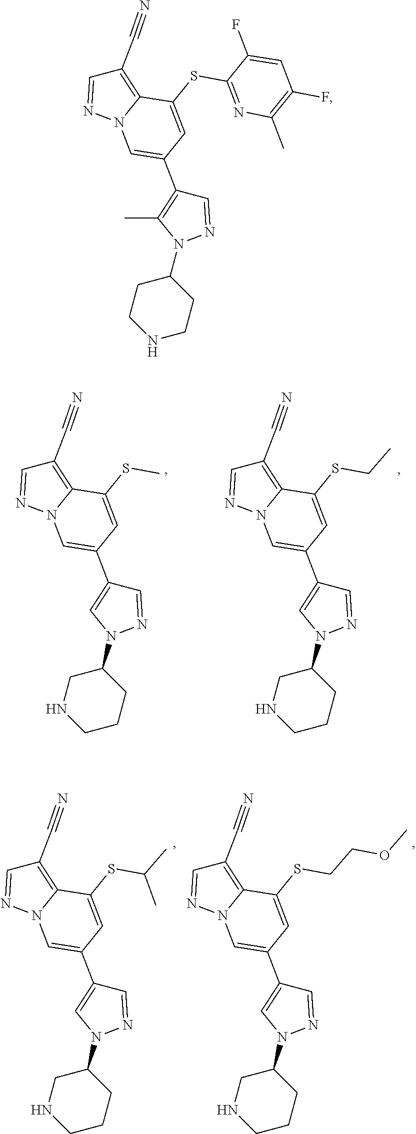

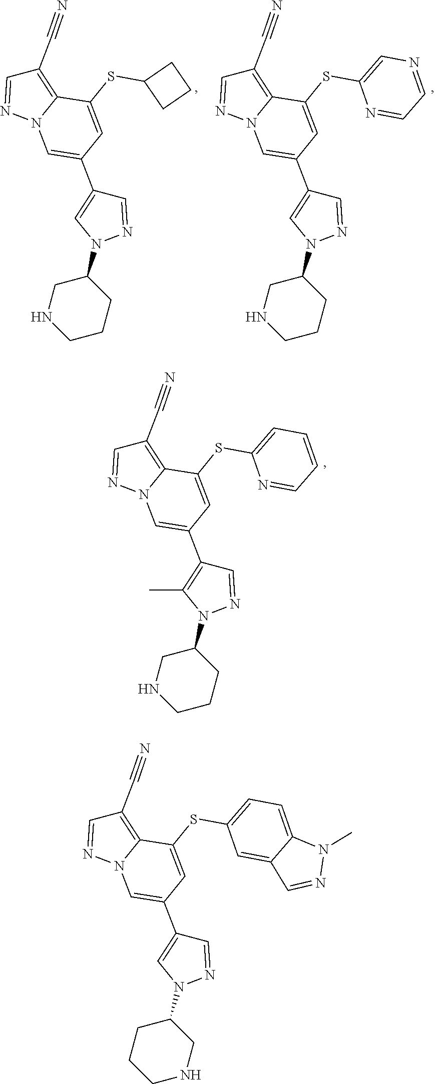

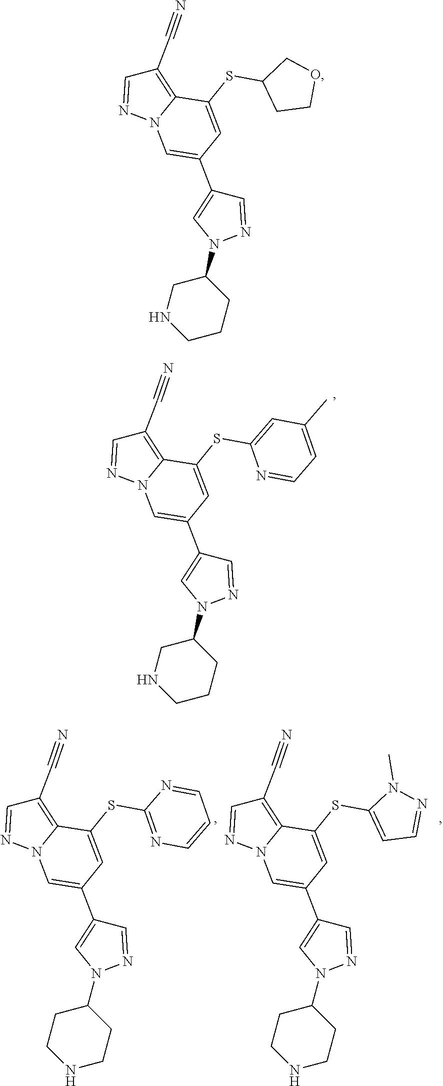

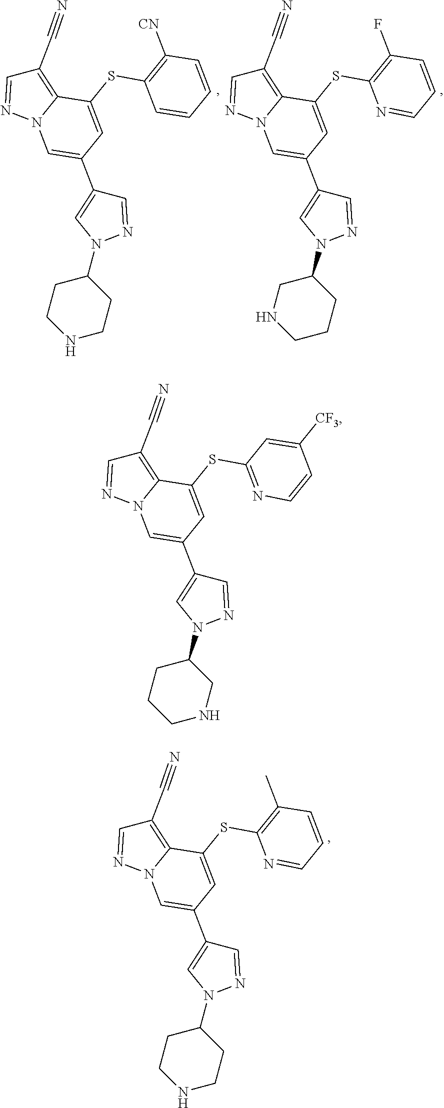

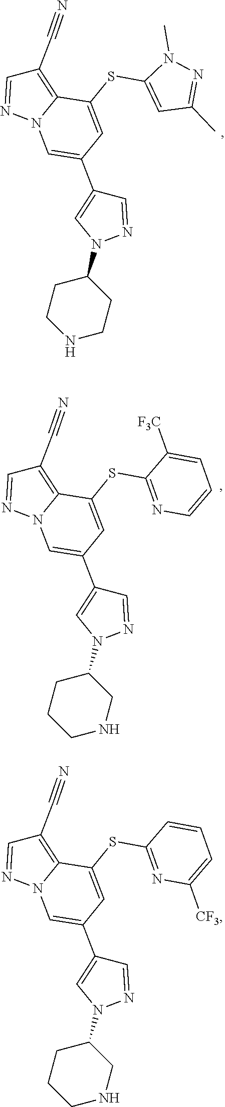

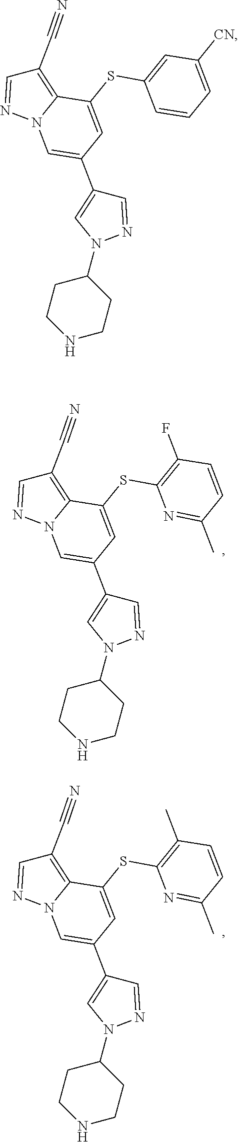 [SMILES: N#Cc1cccc(Sc2cc(-c3cnn(C4CCNCC4)c3)cn3ncc(C#N)c23)c1 ; Cc1ccc(F)c(Sc2cc(-c3cnn(C4CCNCC4)c3)cn3ncc(C#N)c23)n1 ; Cc1ccc(C)c(Sc2cc(-c3cnn(C4CCNCC4)c3)cn3ncc(C#N)c23)n1]

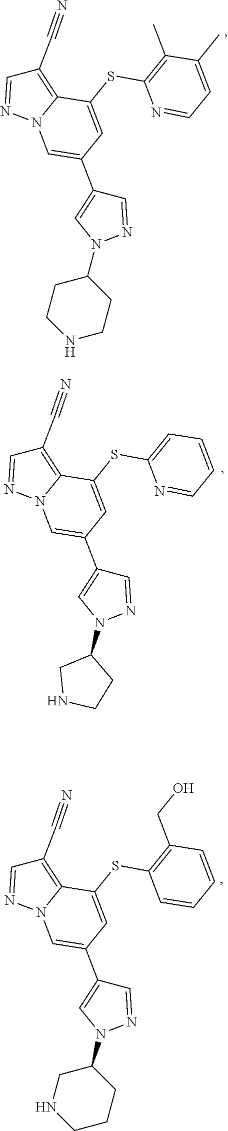

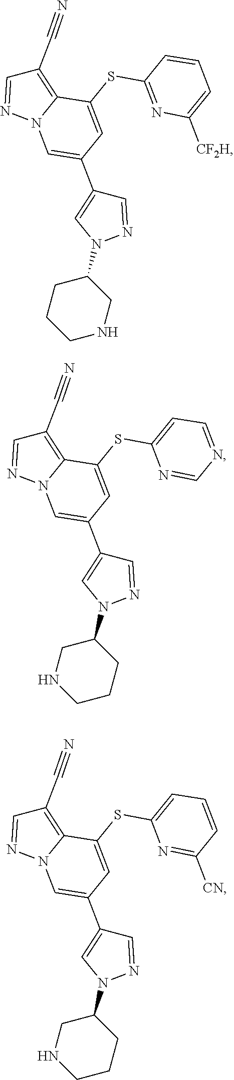

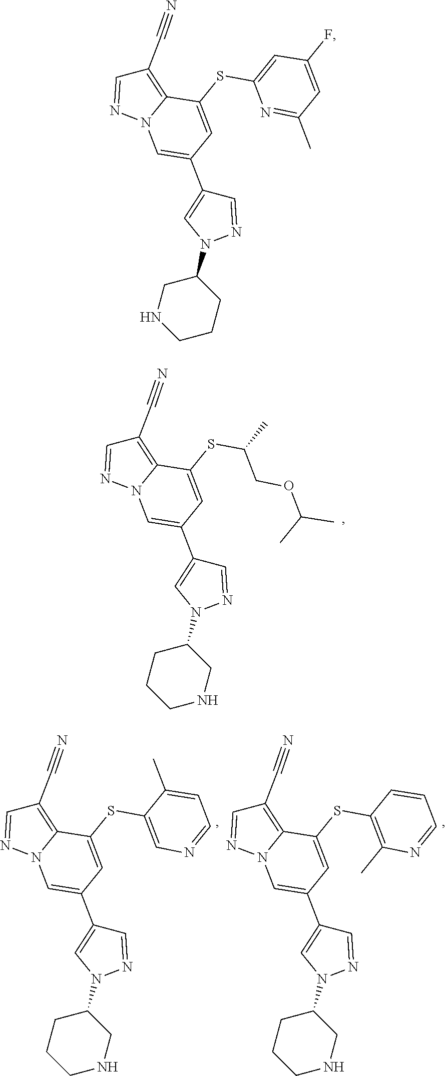

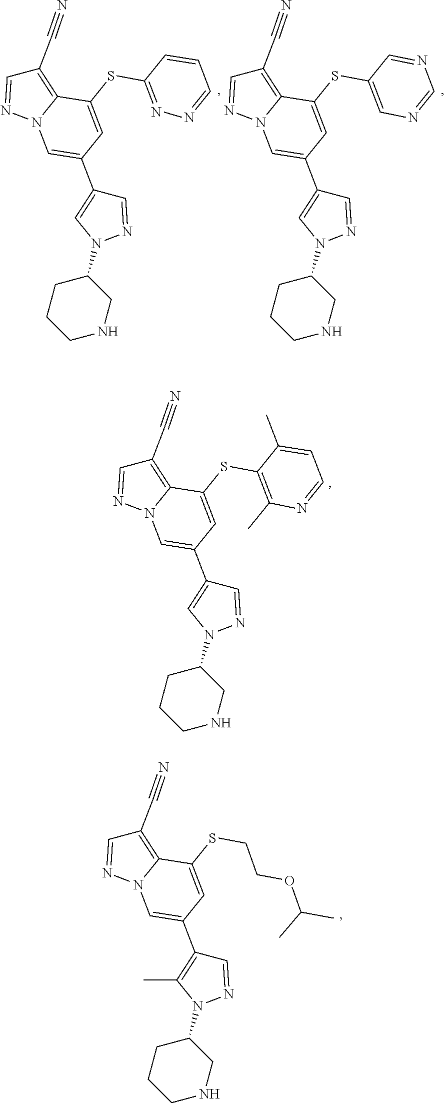

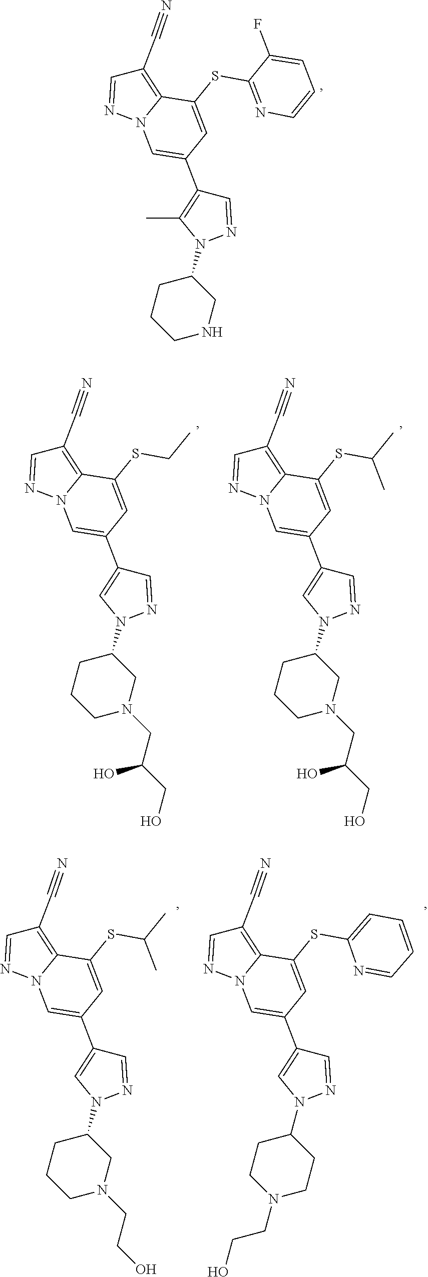 [SMILES: Cc1c(-c2cc(Sc3ncccc3F)c3c(C#N)cnn3c2)cnn1[C@H]1CCCNC1 ; CCSc1cc(-c2cnn([C@H]3CCCN(C[C@H](O)CO)C3)c2)cn2ncc(C#N)c12 ; CC(C)Sc1cc(-c2cnn([C@H]3CCCN(C[C@H](O)CO)C3)c2)cn2ncc(C#N)c12 ; CC(C)Sc1cc(-c2cnn([C@H]3CCCN(CCO)C3)c2)cn2ncc(C#N)c12 ; N#Cc1cnn2cc(-c3cnn(C4CCN(CCO)CC4)c3)cc(Sc3ccccn3)c12]

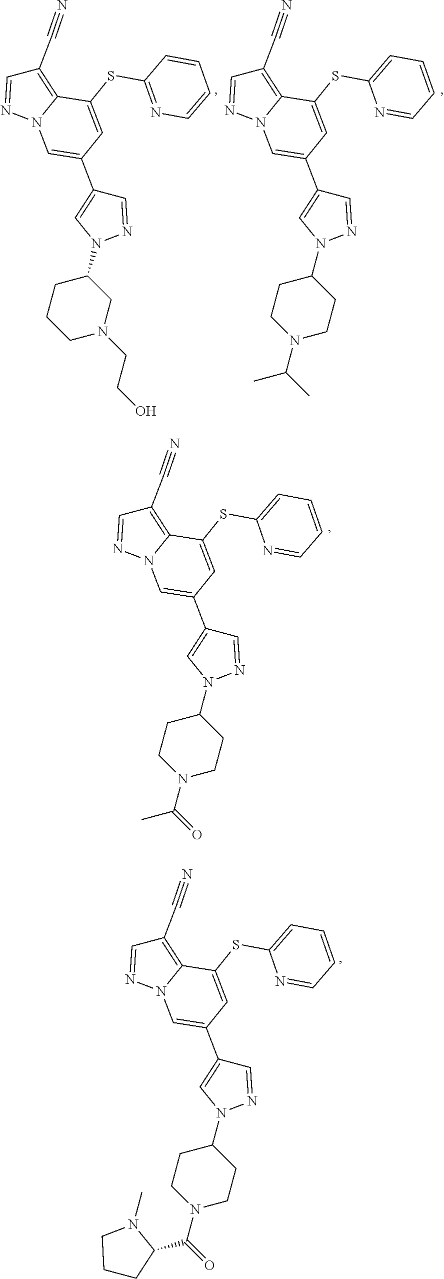

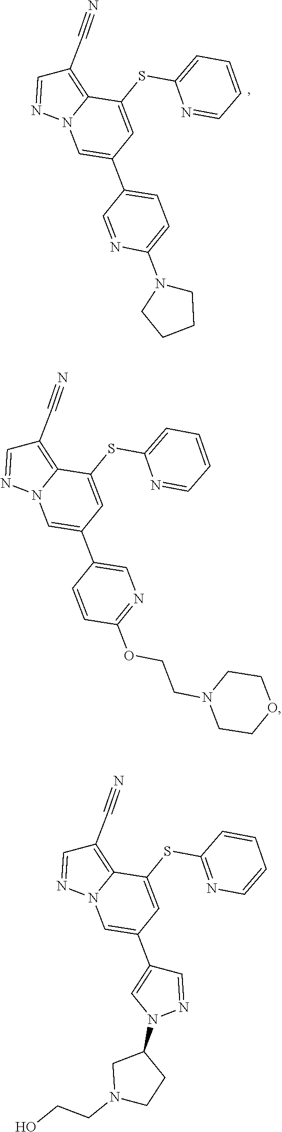 [SMILES: N#Cc1cnn2cc(-c3ccc(N4CCCC4)nc3)cc(Sc3ccccn3)c12 ; N#Cc1cnn2cc(-c3ccc(OCCN4CCOCC4)nc3)cc(Sc3ccccn3)c12 ; N#Cc1cnn2cc(-c3cnn([C@H]4CCN(CCO)C4)c3)cc(Sc3ccccn3)c12]

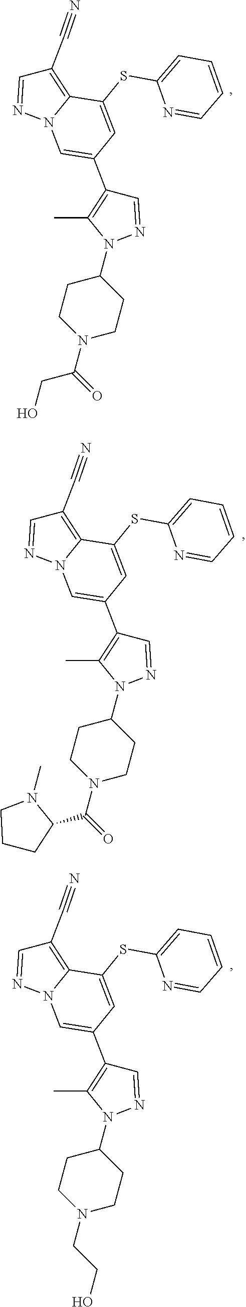 [SMILES: Cc1c(-c2cc(Sc3ccccn3)c3c(C#N)cnn3c2)cnn1C1CCN(C(=O)CO)CC1 ; Cc1c(-c2cc(Sc3ccccn3)c3c(C#N)cnn3c2)cnn1C1CCN(C(=O)[C@@H]2CCCN2C)CC1 ; Cc1c(-c2cc(Sc3ccccn3)c3c(C#N)cnn3c2)cnn1C1CCN(CCO)CC1]

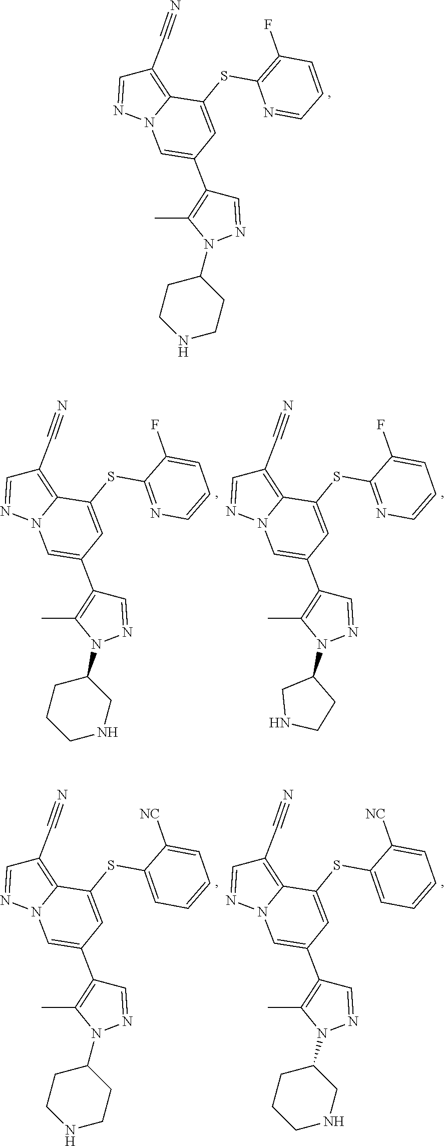

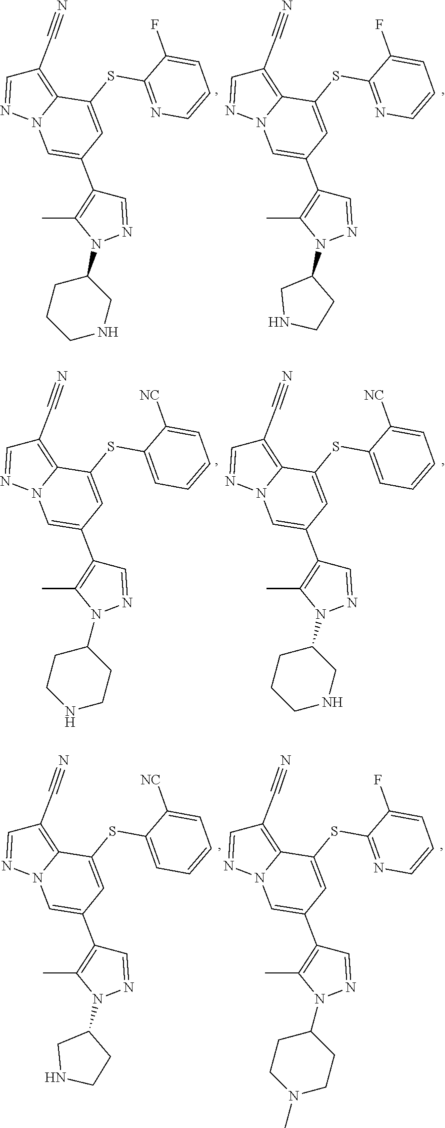

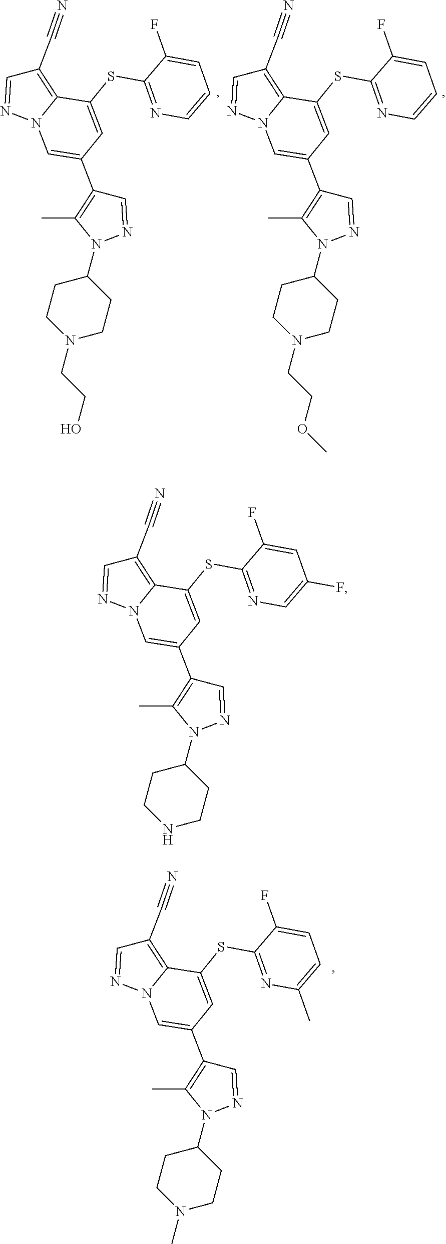

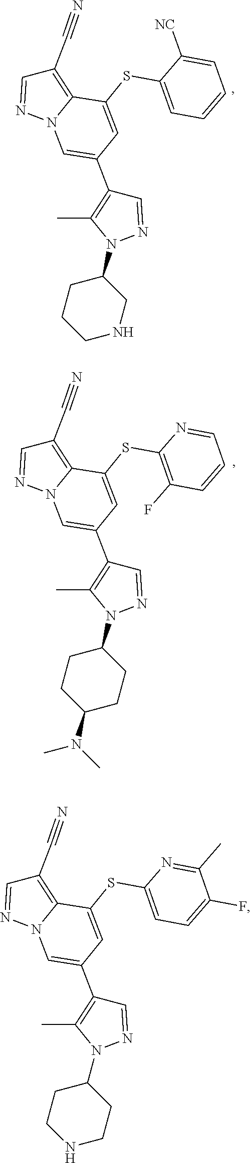

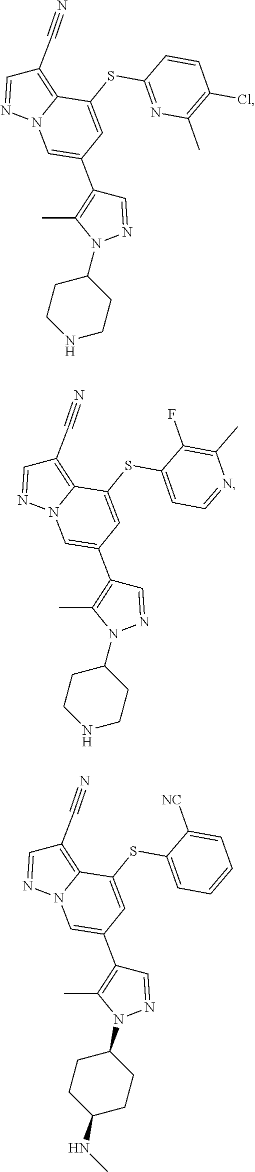 [SMILES: Cc1nc(Sc2cc(-c3cnn(C4CCNCC4)c3C)cn3ncc(C#N)c23)ccc1Cl ; Cc1nccc(Sc2cc(-c3cnn(C4CCNCC4)c3C)cn3ncc(C#N)c23)c1F ; CN[C@H]1CC[C@@H](n2ncc(-c3cc(Sc4ccccc4C#N)c4c(C#N)cnn4c3)c2C)CC1]

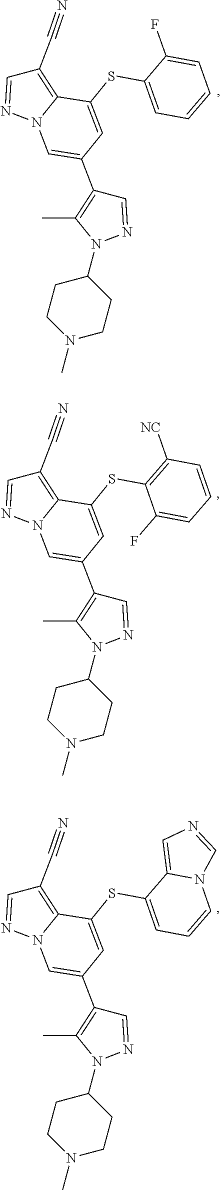

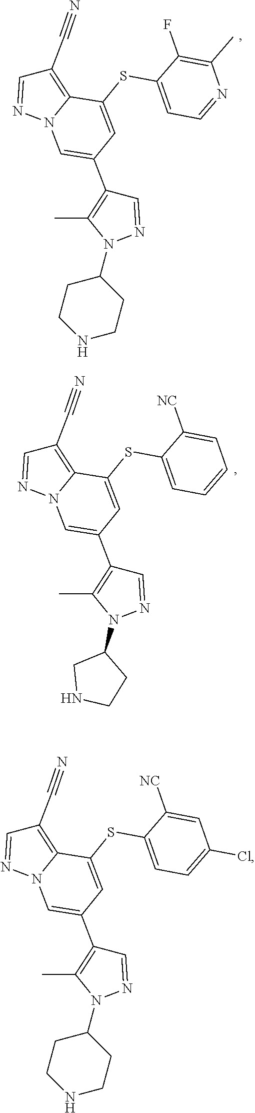

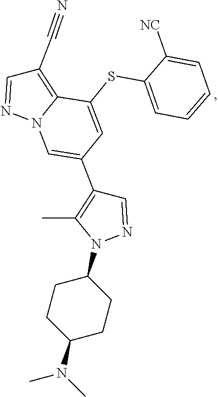 [SMILES: Cc1c(-c2cc(Sc3ccccc3C#N)c3c(C#N)cnn3c2)cnn1[C@H]1CC[C@@H](N(C)C)CC1]

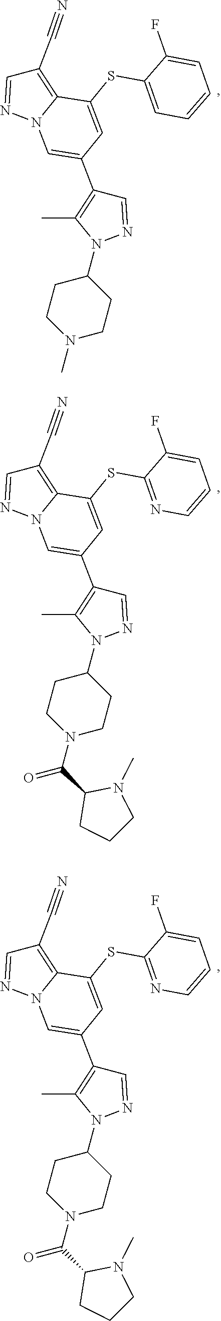

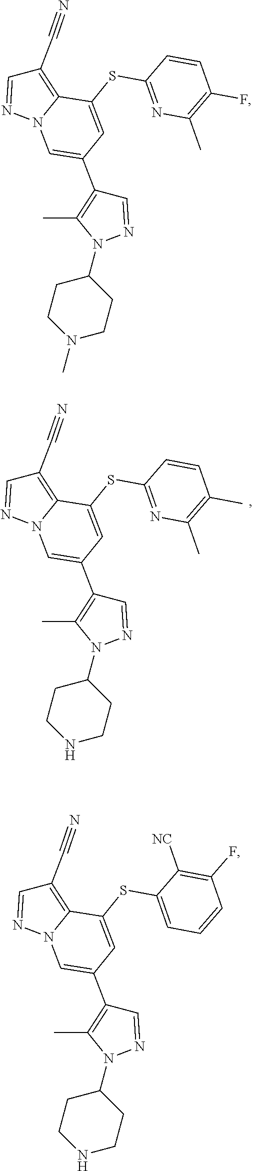

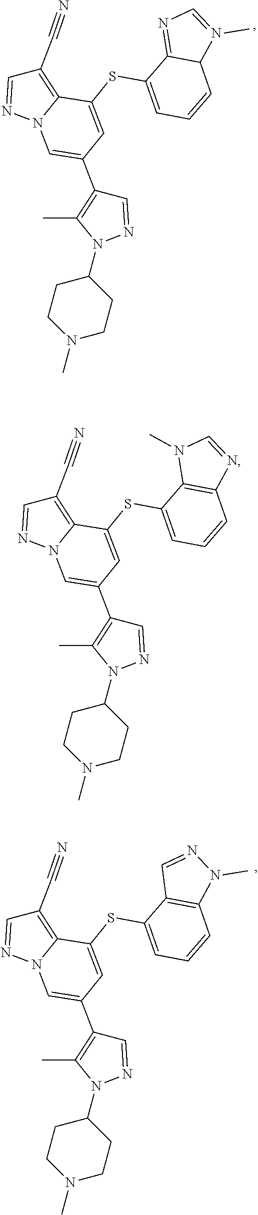

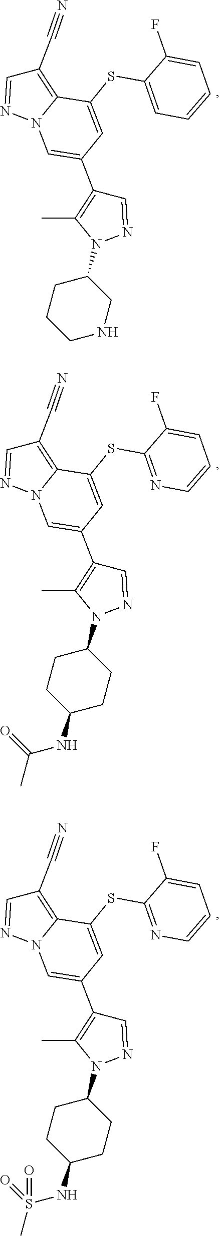

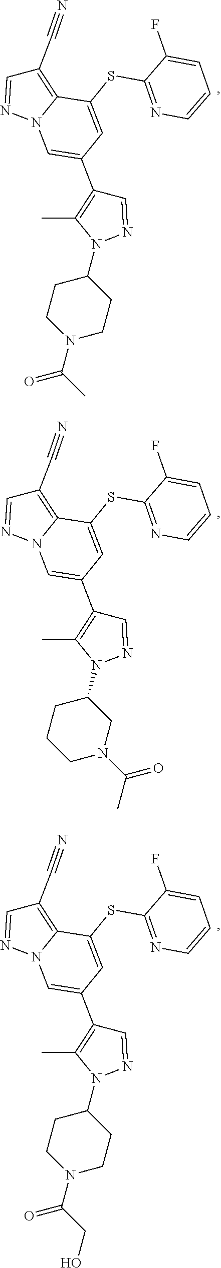

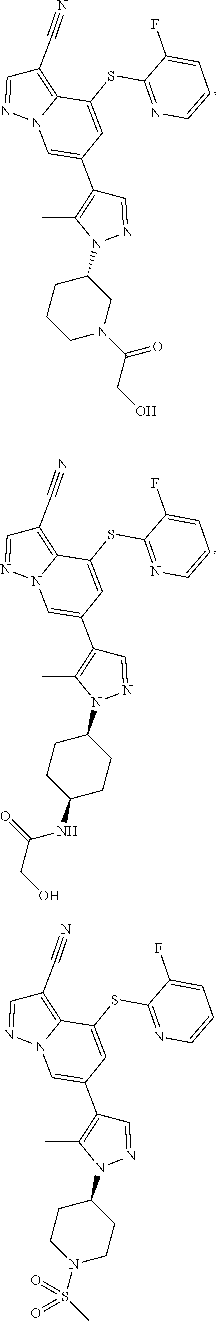

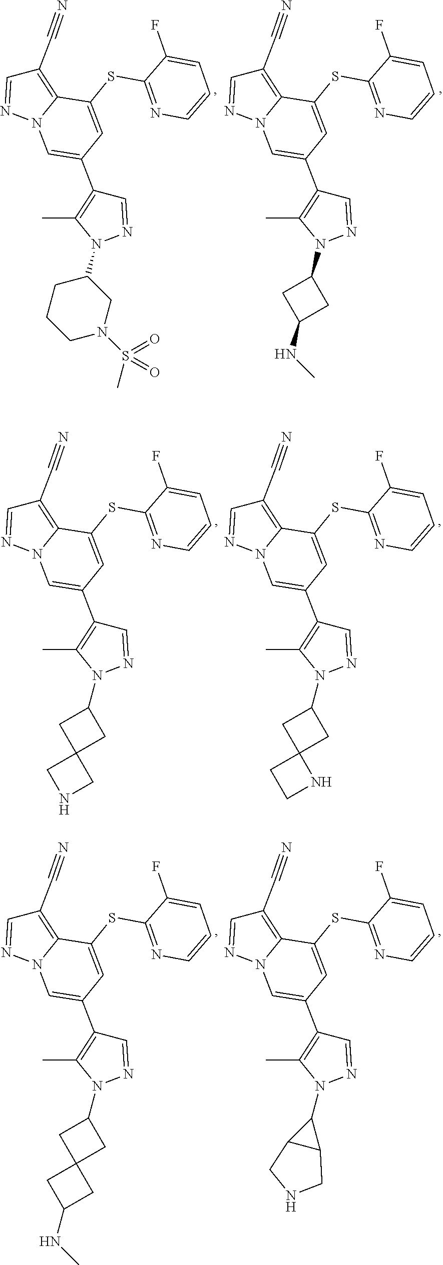

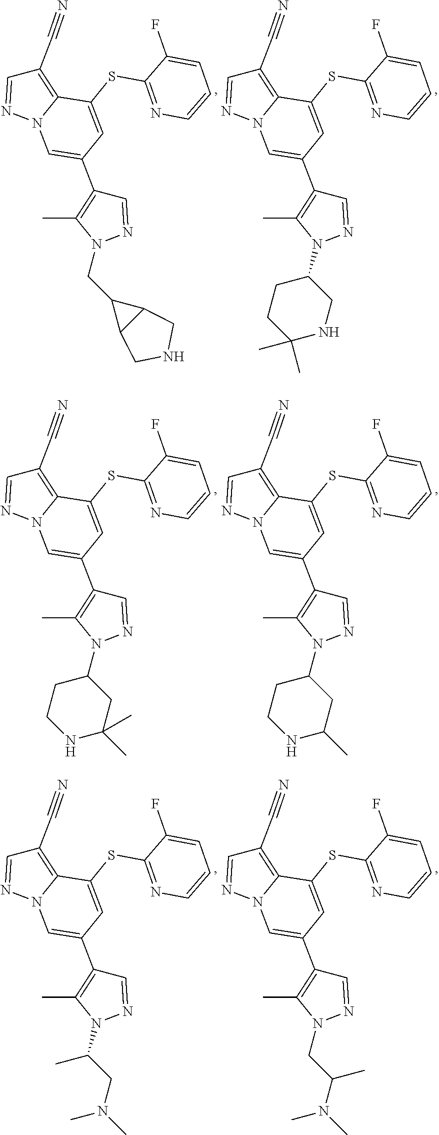

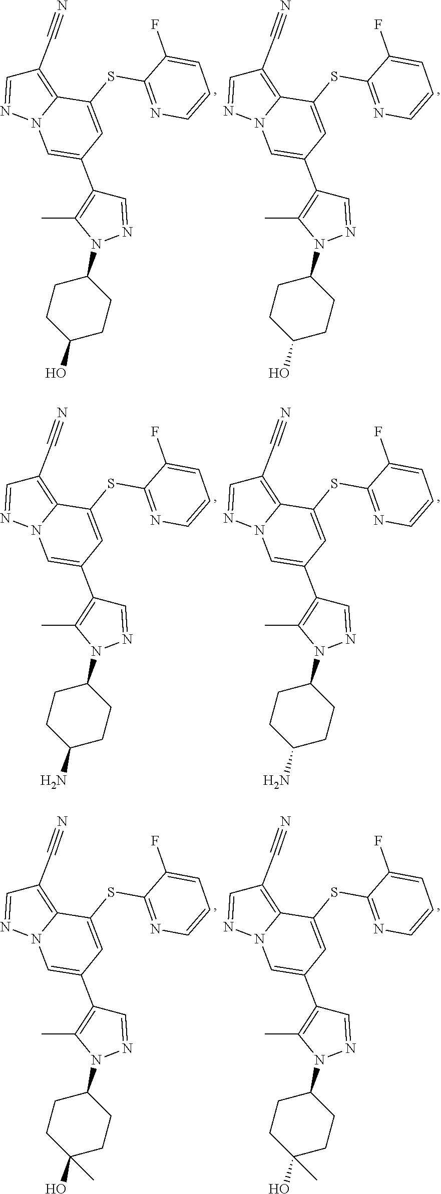

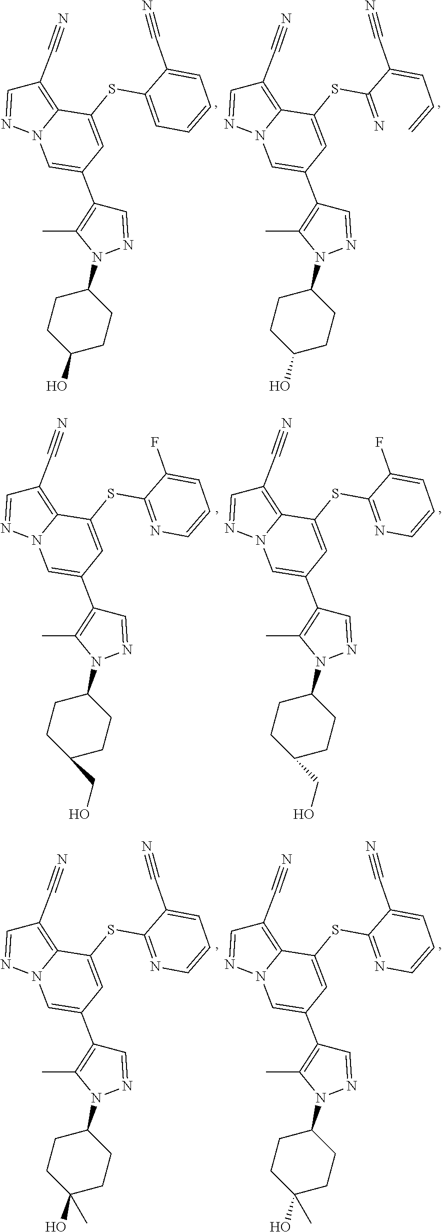

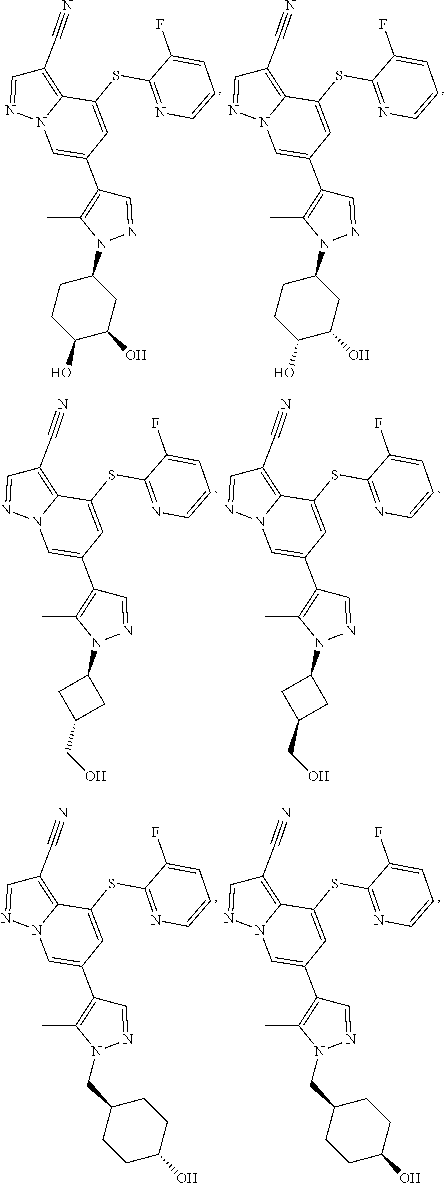

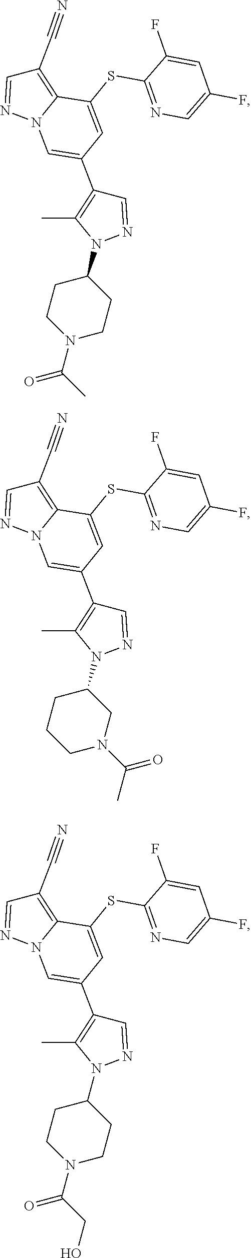

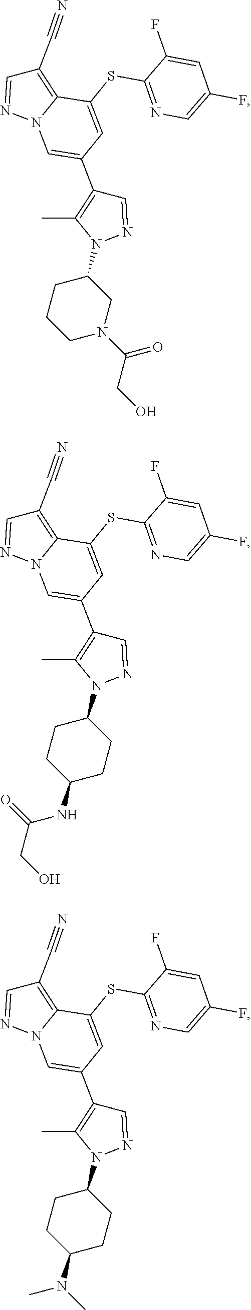

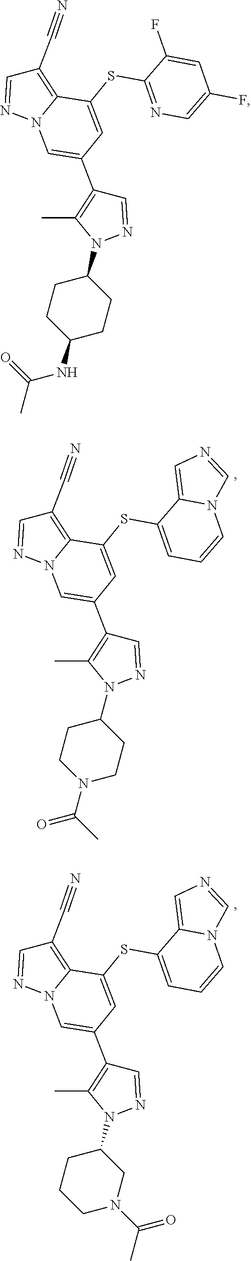

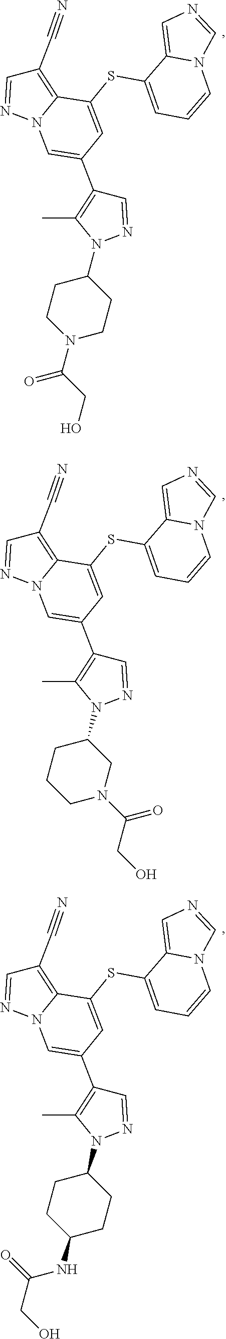

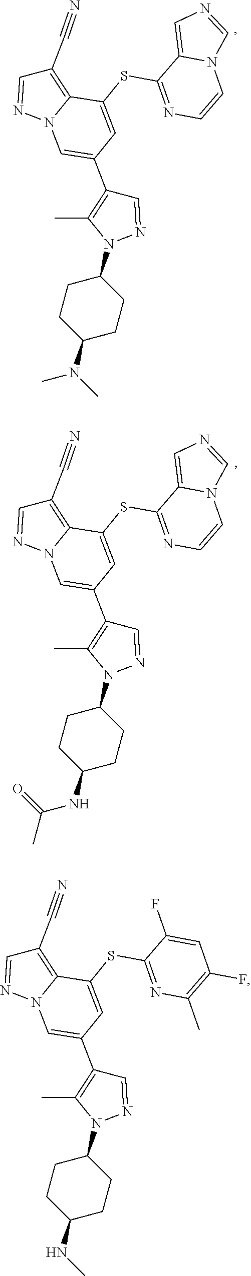

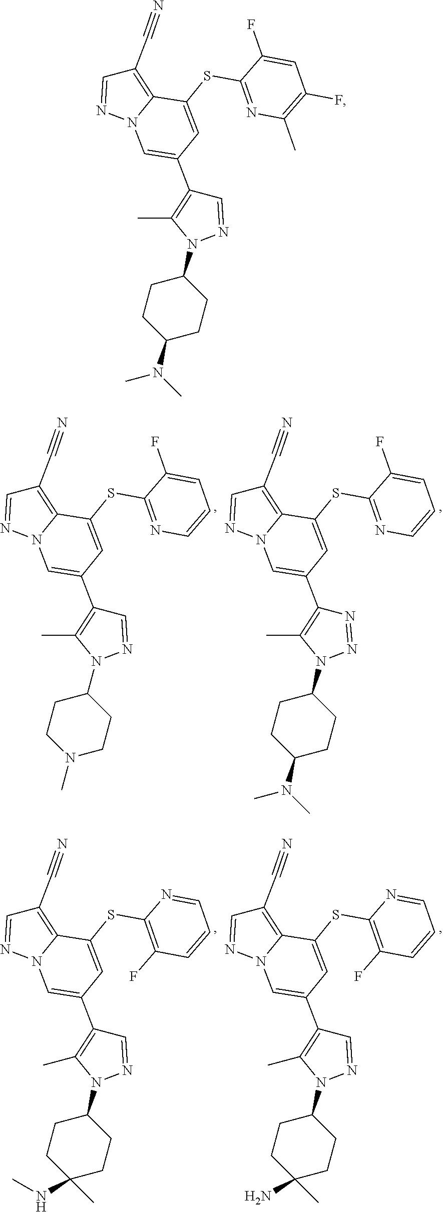

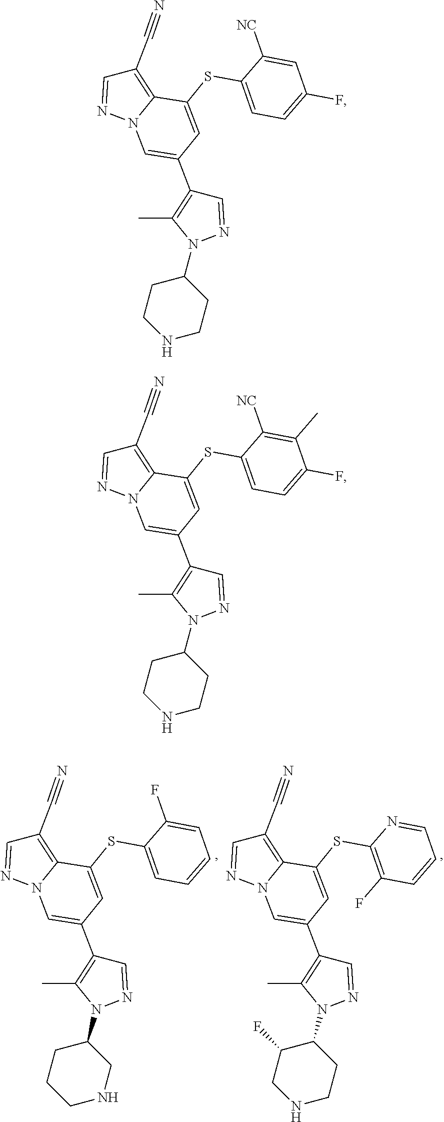

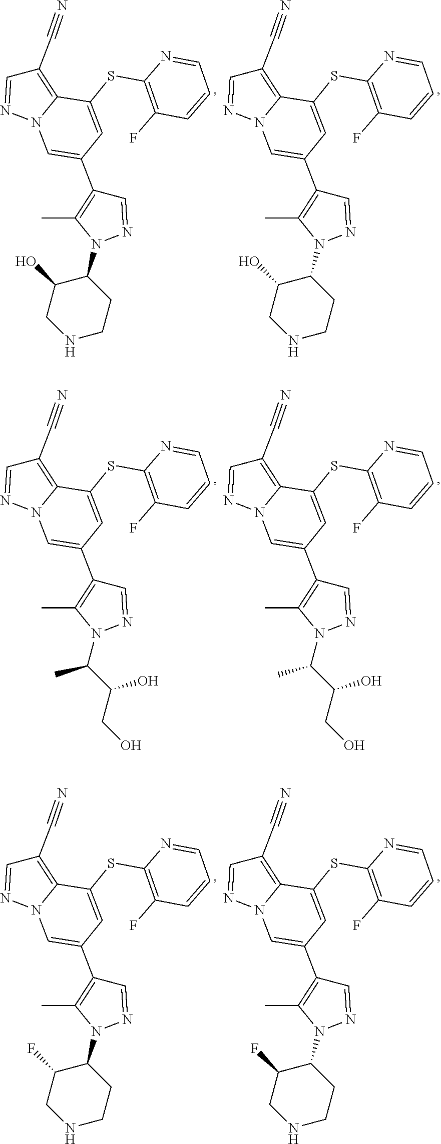

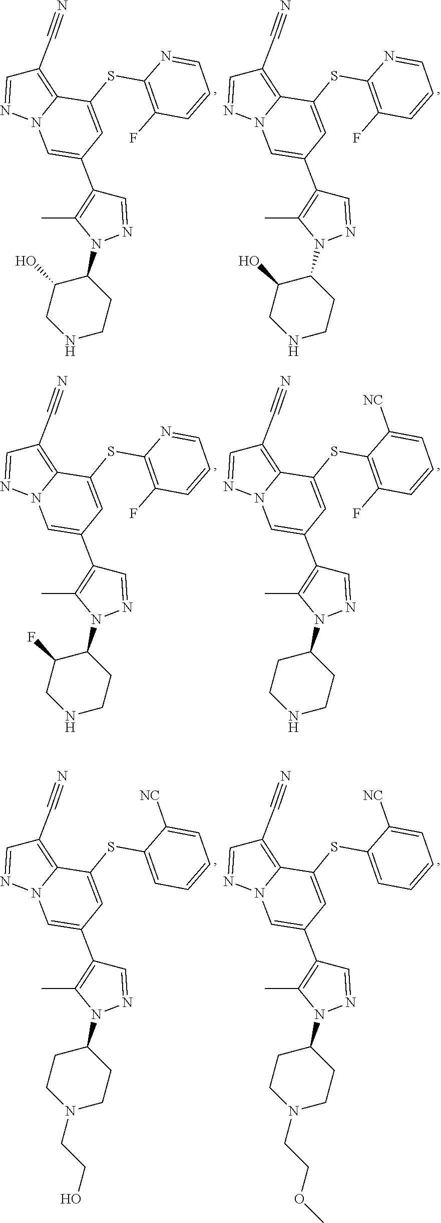

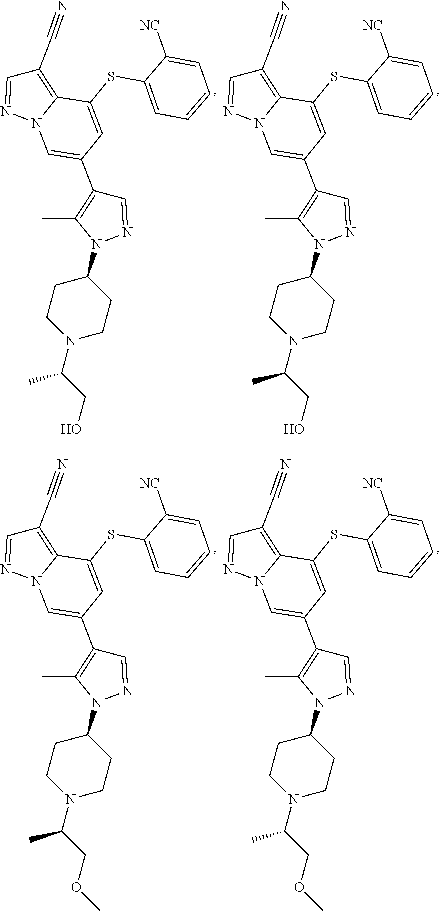

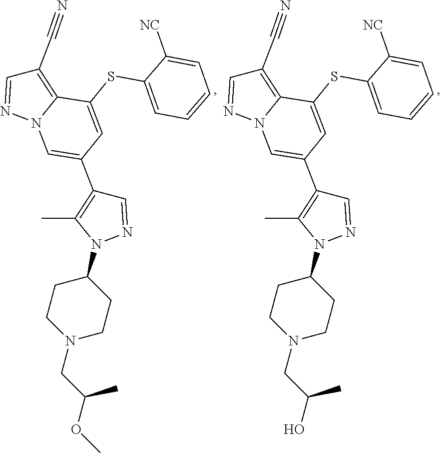

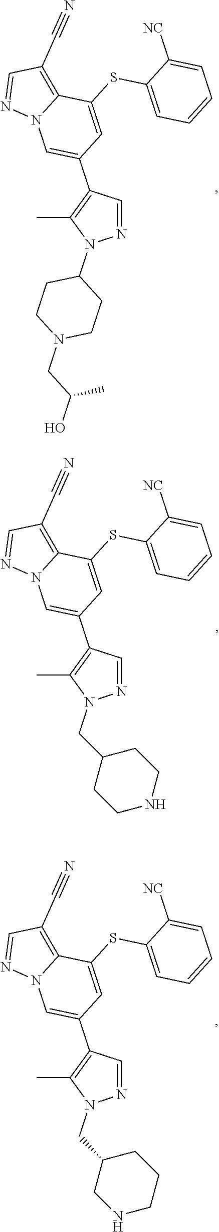

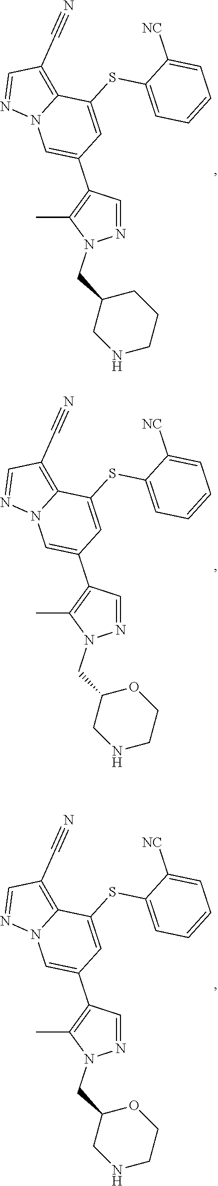 [SMILES: Cc1c(-c2cc(Sc3ccccc3C#N)c3c(C#N)cnn3c2)cnn1C[C@H]1CCCNC1 ; Cc1c(-c2cc(Sc3ccccc3C#N)c3c(C#N)cnn3c2)cnn1C[C@H]1CNCCO1 ; Cc1c(-c2cc(Sc3ccccc3C#N)c3c(C#N)cnn3c2)cnn1C[C@@H]1CNCCO1]

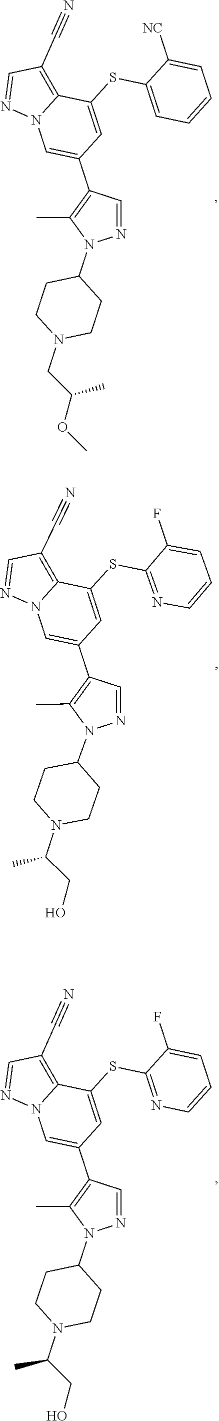

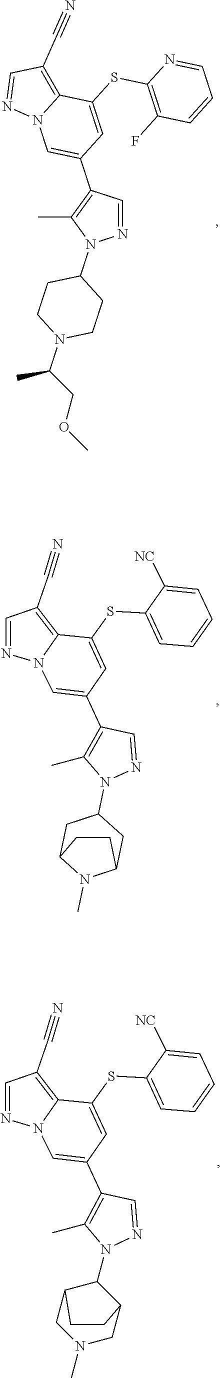 [SMILES: COC[C@@H](C)N1CCC(n2ncc(-c3cc(Sc4ncccc4F)c4c(C#N)cnn4c3)c2C)CC1 ; Cc1c(-c2cc(Sc3ccccc3C#N)c3c(C#N)cnn3c2)cnn1C1CC2CCC(C1)N2C ; Cc1c(-c2cc(Sc3ccccc3C#N)c3c(C#N)cnn3c2)cnn1C1C2CCC1CN(C)C2]

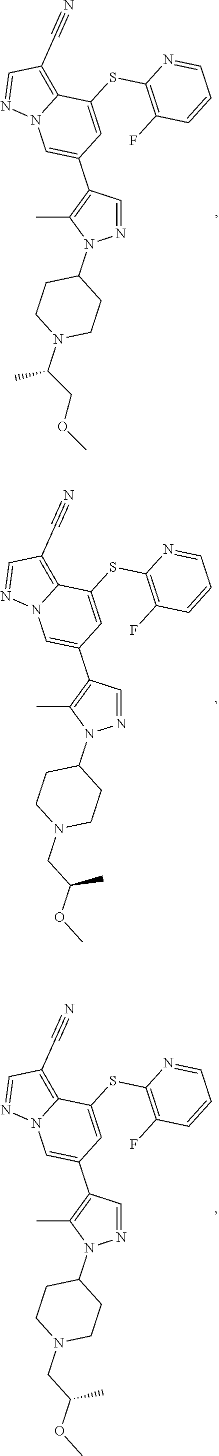 [SMILES: COC[C@H](C)N1CCC(n2ncc(-c3cc(Sc4ncccc4F)c4c(C#N)cnn4c3)c2C)CC1 ; CO[C@H](C)CN1CCC(n2ncc(-c3cc(Sc4ncccc4F)c4c(C#N)cnn4c3)c2C)CC1 ; CO[C@@H](C)CN1CCC(n2ncc(-c3cc(Sc4ncccc4F)c4c(C#N)cnn4c3)c2C)CC1]

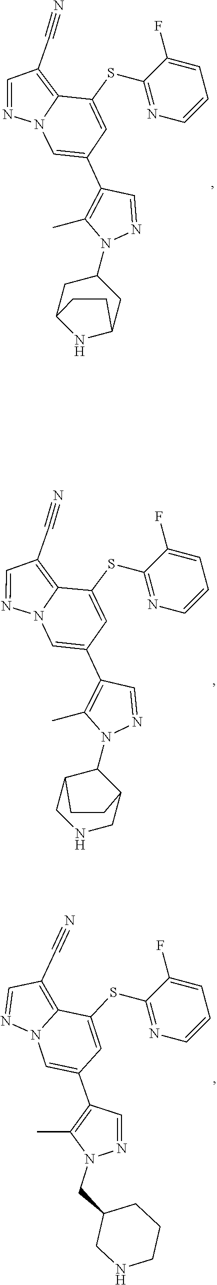 [SMILES: Cc1c(-c2cc(Sc3ncccc3F)c3c(C#N)cnn3c2)cnn1C1CC2CCC(C1)N2 ; Cc1c(-c2cc(Sc3ncccc3F)c3c(C#N)cnn3c2)cnn1C1C2CCC1CNC2 ; Cc1c(-c2cc(Sc3ncccc3F)c3c(C#N)cnn3c2)cnn1C[C@H]1CCCNC1]

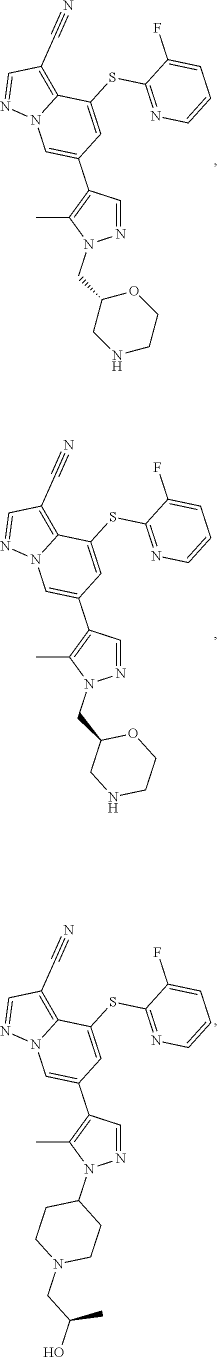 [SMILES: Cc1c(-c2cc(Sc3ncccc3F)c3c(C#N)cnn3c2)cnn1C[C@H]1CNCCO1 ; Cc1c(-c2cc(Sc3ncccc3F)c3c(C#N)cnn3c2)cnn1C[C@@H]1CNCCO1 ; Cc1c(-c2cc(Sc3ncccc3F)c3c(C#N)cnn3c2)cnn1C1CCN(C[C@@H](C)O)CC1]

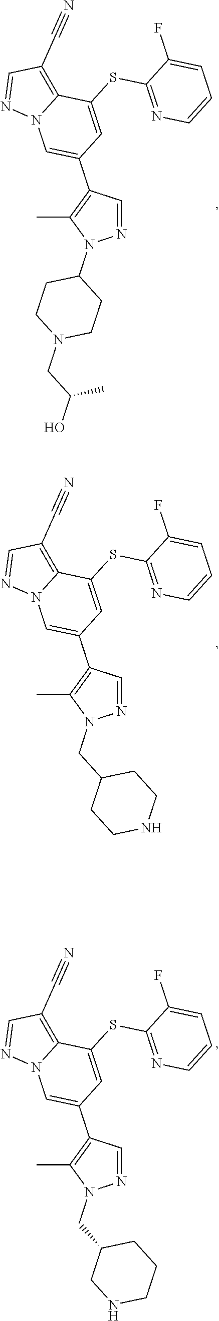 [SMILES: Cc1c(-c2cc(Sc3ncccc3F)c3c(C#N)cnn3c2)cnn1C1CCN(C[C@H](C)O)CC1 ; Cc1c(-c2cc(Sc3ncccc3F)c3c(C#N)cnn3c2)cnn1CC1CCNCC1 ; Cc1c(-c2cc(Sc3ncccc3F)c3c(C#N)cnn3c2)cnn1C[C@@H]1CCCNC1]

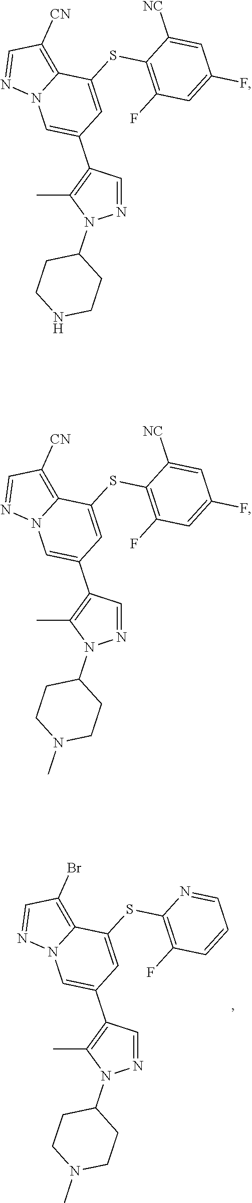 [SMILES: Cc1c(-c2cc(Sc3c(F)cc(F)cc3C#N)c3c(C#N)cnn3c2)cnn1C1CCNCC1 ; Cc1c(-c2cc(Sc3c(F)cc(F)cc3C#N)c3c(C#N)cnn3c2)cnn1C1CCN(C)CC1 ; Cc1c(-c2cc(Sc3ncccc3F)c3c(Br)cnn3c2)cnn1C1CCN(C)CC1]

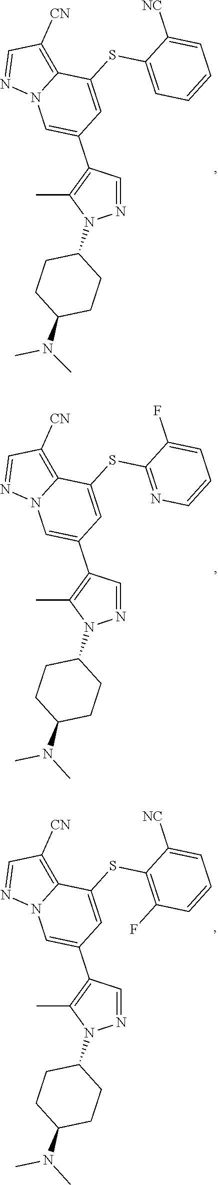 [SMILES: Cc1c(-c2cc(Sc3ccccc3C#N)c3c(C#N)cnn3c2)cnn1[C@H]1CC[C@H](N(C)C)CC1 ; Cc1c(-c2cc(Sc3ncccc3F)c3c(C#N)cnn3c2)cnn1[C@H]1CC[C@H](N(C)C)CC1 ; Cc1c(-c2cc(Sc3c(F)cccc3C#N)c3c(C#N)cnn3c2)cnn1[C@H]1CC[C@H](N(C)C)CC1]

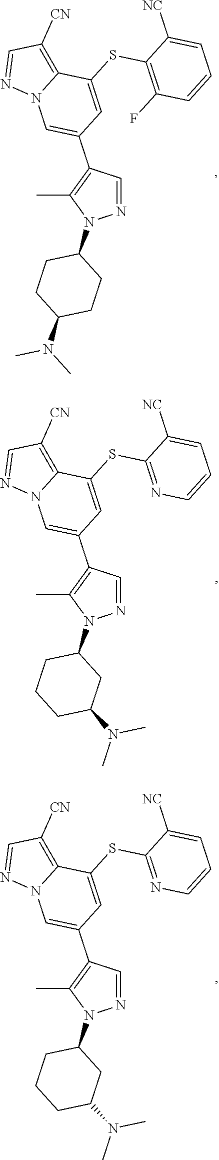

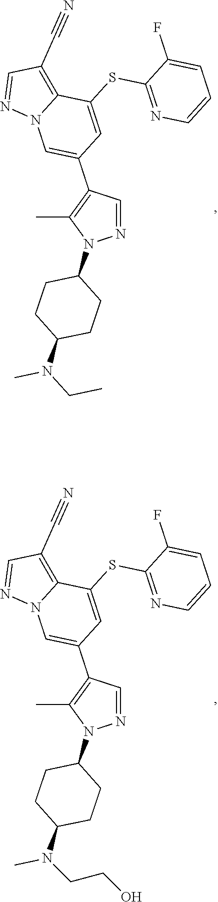 [SMILES: CCN(C)[C@H]1CC[C@@H](n2ncc(-c3cc(Sc4ncccc4F)c4c(C#N)cnn4c3)c2C)CC1 ; Cc1c(-c2cc(Sc3ncccc3F)c3c(C#N)cnn3c2)cnn1[C@H]1CC[C@@H](N(C)CCO)CC1]

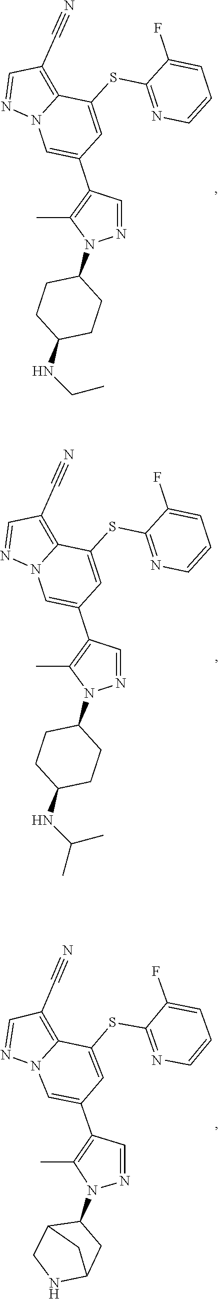 [SMILES: CCN[C@H]1CC[C@@H](n2ncc(-c3cc(Sc4ncccc4F)c4c(C#N)cnn4c3)c2C)CC1 ; Cc1c(-c2cc(Sc3ncccc3F)c3c(C#N)cnn3c2)cnn1[C@H]1CC[C@@H](NC(C)C)CC1 ; Cc1c(-c2cc(Sc3ncccc3F)c3c(C#N)cnn3c2)cnn1[C@H]1CC2CC1CN2]

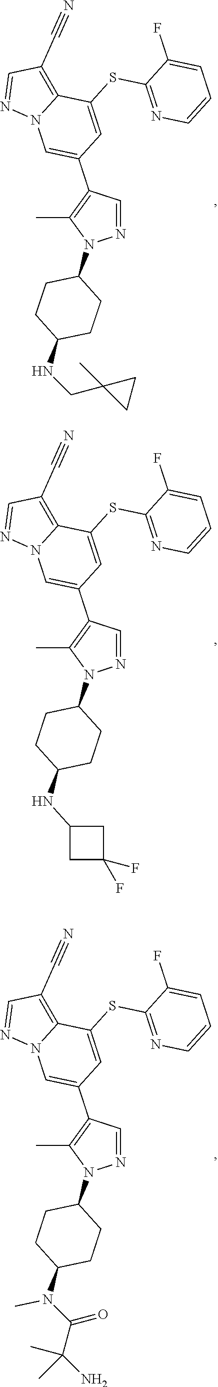

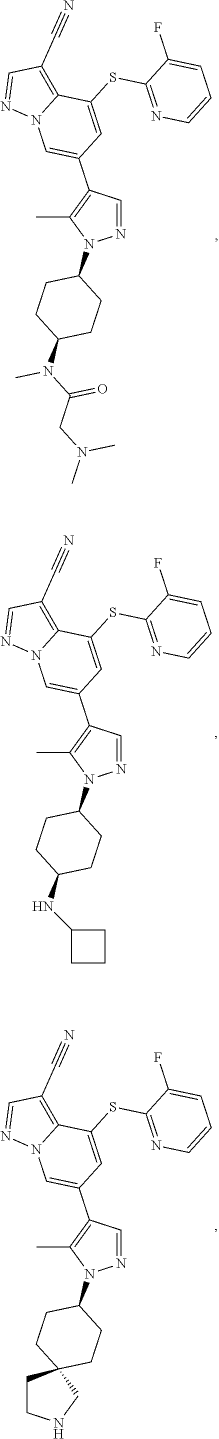 [SMILES: Cc1c(-c2cc(Sc3ncccc3F)c3c(C#N)cnn3c2)cnn1[C@H]1CC[C@@H](N(C)C(=O)CN(C)C)CC1 ; Cc1c(-c2cc(Sc3ncccc3F)c3c(C#N)cnn3c2)cnn1[C@H]1CC[C@@H](NC2CCC2)CC1 ; Cc1c(-c2cc(Sc3ncccc3F)c3c(C#N)cnn3c2)cnn1[C@H]1CC[C@@]2(CCNC2)CC1]

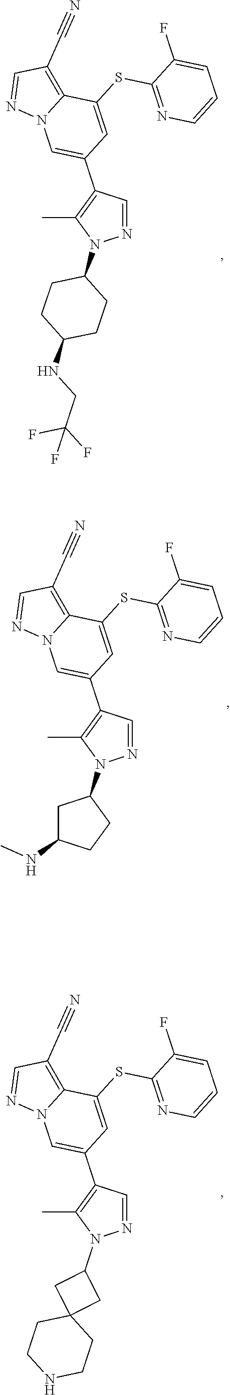 [SMILES: Cc1c(-c2cc(Sc3ncccc3F)c3c(C#N)cnn3c2)cnn1[C@H]1CC[C@@H](NCC(F)(F)F)CC1 ; CN[C@@H]1CC[C@H](n2ncc(-c3cc(Sc4ncccc4F)c4c(C#N)cnn4c3)c2C)C1 ; Cc1c(-c2cc(Sc3ncccc3F)c3c(C#N)cnn3c2)cnn1C1CC2(CCNCC2)C1]

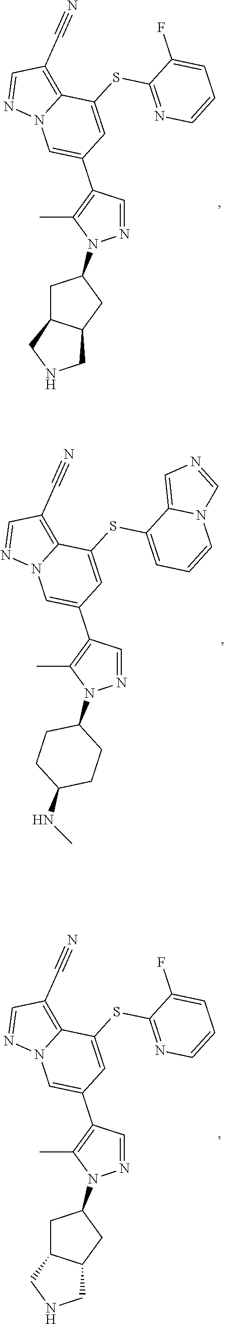 [SMILES: Cc1c(-c2cc(Sc3ncccc3F)c3c(C#N)cnn3c2)cnn1[C@H]1C[C@H]2CNC[C@H]2C1 ; CN[C@H]1CC[C@@H](n2ncc(-c3cc(Sc4cccn5cncc45)c4c(C#N)cnn4c3)c2C)CC1 ; Cc1c(-c2cc(Sc3ncccc3F)c3c(C#N)cnn3c2)cnn1[C@@H]1C[C@H]2CNC[C@H]2C1]

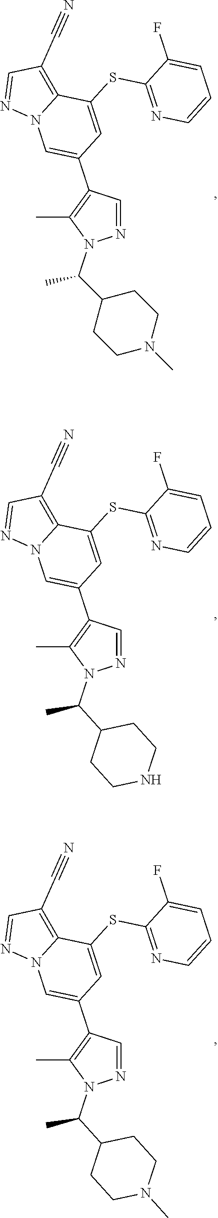 [SMILES: Cc1c(-c2cc(Sc3ncccc3F)c3c(C#N)cnn3c2)cnn1[C@@H](C)C1CCN(C)CC1 ; Cc1c(-c2cc(Sc3ncccc3F)c3c(C#N)cnn3c2)cnn1[C@H](C)C1CCNCC1 ; Cc1c(-c2cc(Sc3ncccc3F)c3c(C#N)cnn3c2)cnn1[C@H](C)C1CCN(C)CC1]

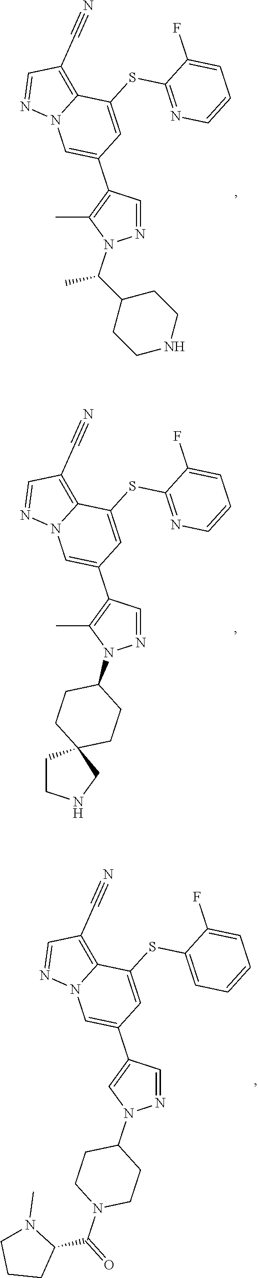 [SMILES: Cc1c(-c2cc(Sc3ncccc3F)c3c(C#N)cnn3c2)cnn1[C@@H](C)C1CCNCC1 ; Cc1c(-c2cc(Sc3ncccc3F)c3c(C#N)cnn3c2)cnn1[C@H]1CC[C@]2(CCNC2)CC1 ; CN1CCC[C@H]1C(=O)N1CCC(n2cc(-c3cc(Sc4ccccc4F)c4c(C#N)cnn4c3)cn2)CC1]

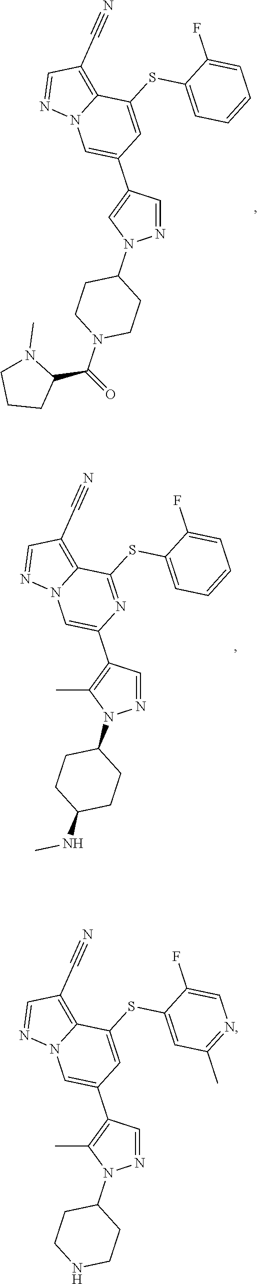

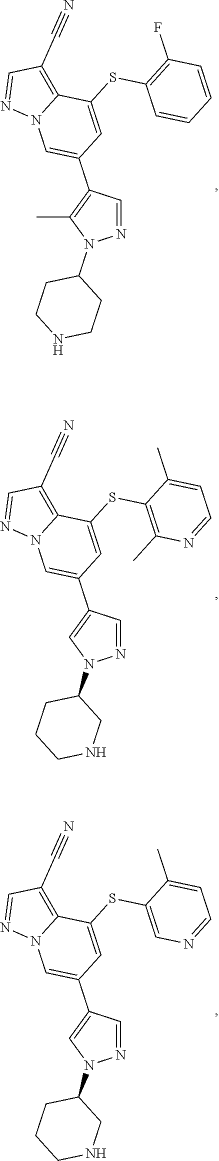 [SMILES: Cc1c(-c2cc(Sc3ccccc3F)c3c(C#N)cnn3c2)cnn1C1CCNCC1 ; Cc1ccnc(C)c1Sc1cc(-c2cnn([C@@H]3CCCNC3)c2)cn2ncc(C#N)c12 ; Cc1ccncc1Sc1cc(-c2cnn([C@@H]3CCCNC3)c2)cn2ncc(C#N)c12]

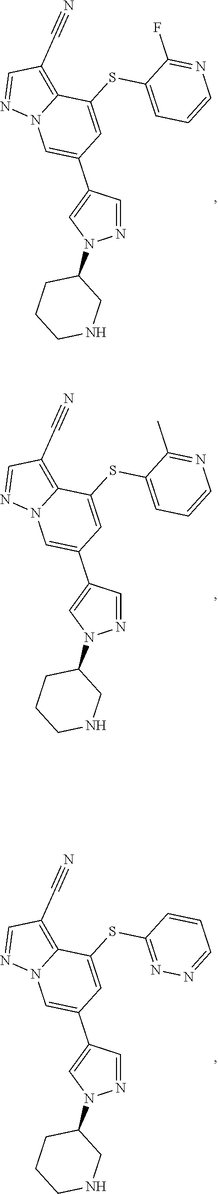 [SMILES: N#Cc1cnn2cc(-c3cnn([C@@H]4CCCNC4)c3)cc(Sc3cccnc3F)c12 ; Cc1ncccc1Sc1cc(-c2cnn([C@@H]3CCCNC3)c2)cn2ncc(C#N)c12 ; N#Cc1cnn2cc(-c3cnn([C@@H]4CCCNC4)c3)cc(Sc3cccnn3)c12]

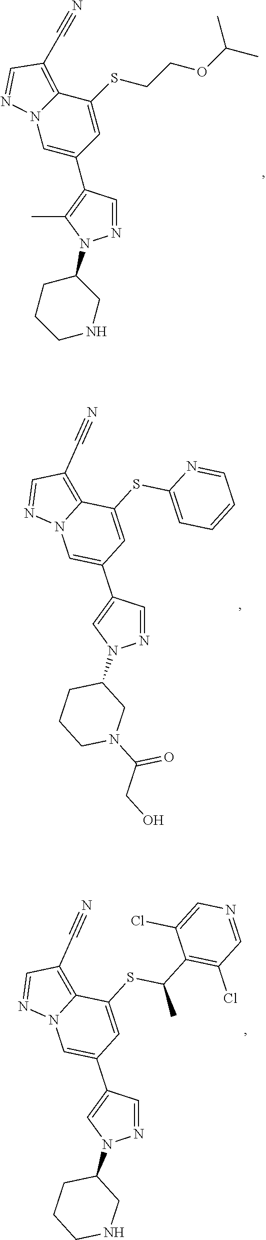 [SMILES: Cc1c(-c2cc(SCCOC(C)C)c3c(C#N)cnn3c2)cnn1[C@@H]1CCCNC1 ; N#Cc1cnn2cc(-c3cnn([C@H]4CCCN(C(=O)CO)C4)c3)cc(Sc3ccccn3)c12 ; C[C@@H](Sc1cc(-c2cnn([C@@H]3CCCNC3)c2)cn2ncc(C#N)c12)c1c(Cl)cncc1Cl]

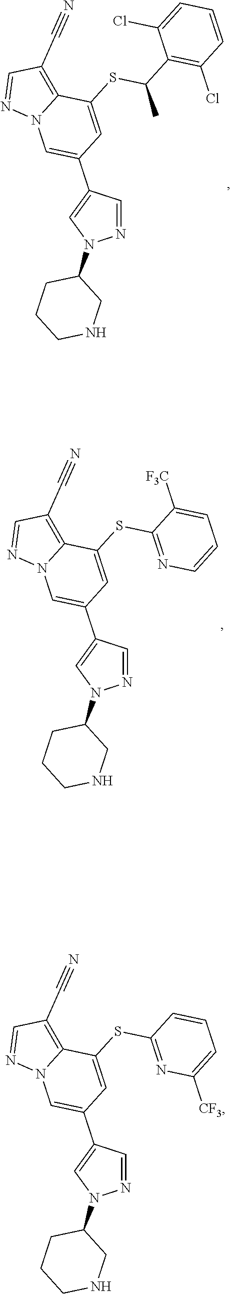 [SMILES: C[C@@H](Sc1cc(-c2cnn([C@@H]3CCCNC3)c2)cn2ncc(C#N)c12)c1c(Cl)cccc1Cl ; N#Cc1cnn2cc(-c3cnn([C@@H]4CCCNC4)c3)cc(Sc3ncccc3C(F)(F)F)c12 ; N#Cc1cnn2cc(-c3cnn([C@@H]4CCCNC4)c3)cc(Sc3cccc(C(F)(F)F)n3)c12]

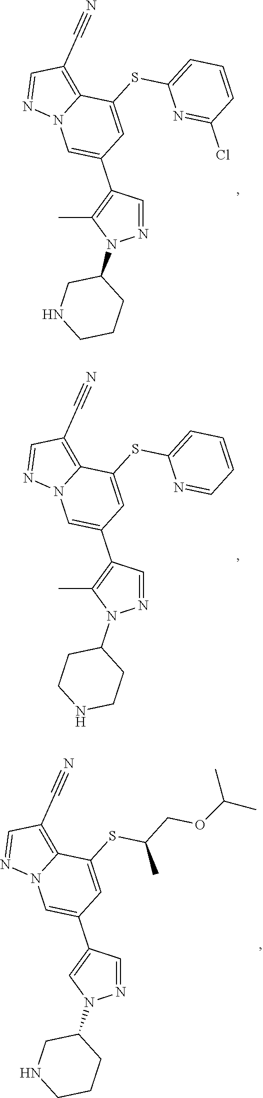

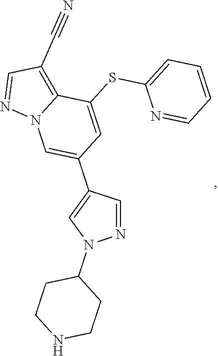 [SMILES: N#Cc1cnn2cc(-c3cnn(C4CCNCC4)c3)cc(Sc3ccccn3)c12]

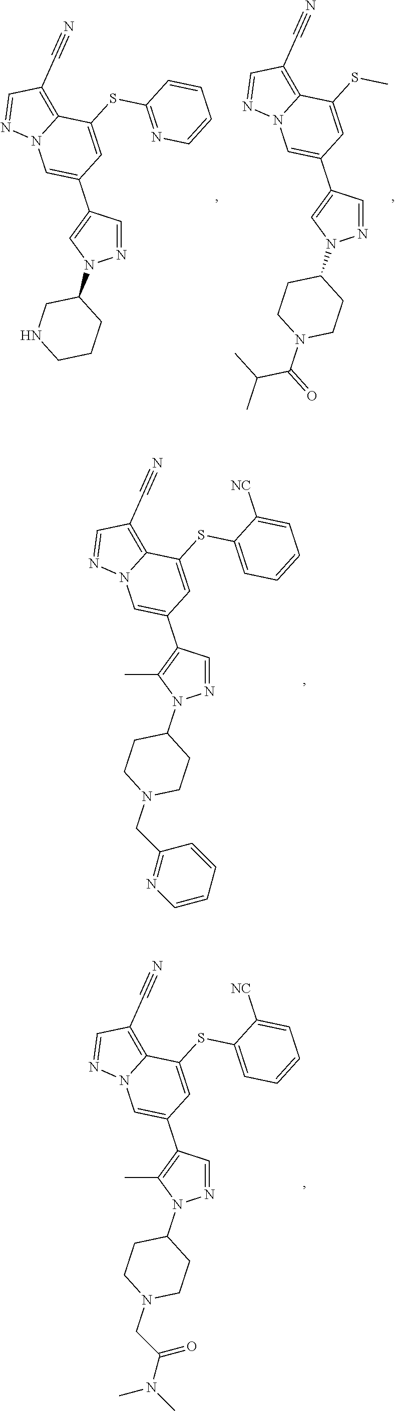

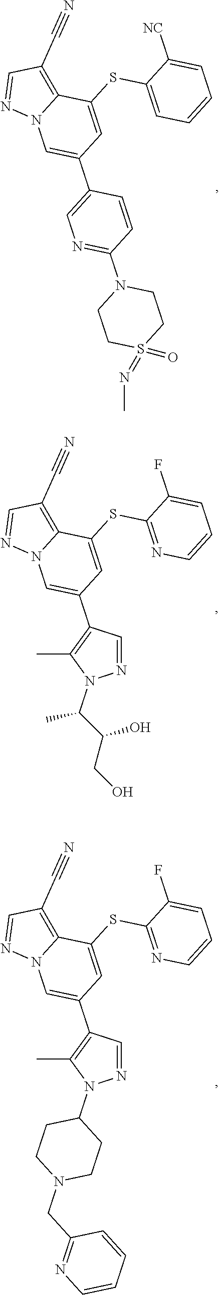 [SMILES: CN=S1(=O)CCN(c2ccc(-c3cc(Sc4ccccc4C#N)c4c(C#N)cnn4c3)cn2)CC1 ; Cc1c(-c2cc(Sc3ncccc3F)c3c(C#N)cnn3c2)cnn1[C@@H](C)[C@H](O)CO ; Cc1c(-c2cc(Sc3ncccc3F)c3c(C#N)cnn3c2)cnn1C1CCN(Cc2ccccn2)CC1]

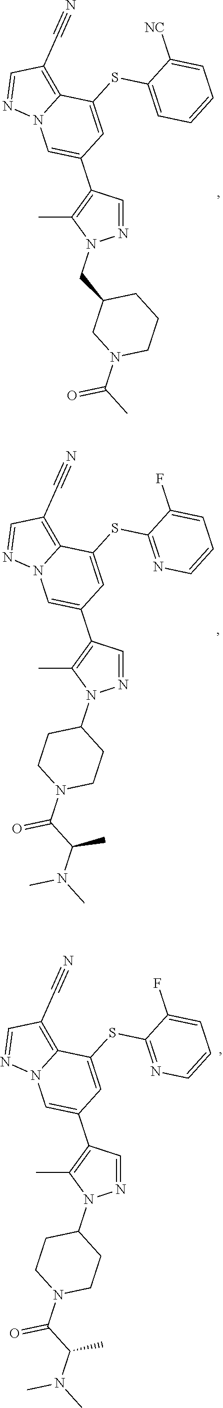 [SMILES: CC(=O)N1CCC[C@H](Cn2ncc(-c3cc(Sc4ccccc4C#N)c4c(C#N)cnn4c3)c2C)C1 ; Cc1c(-c2cc(Sc3ncccc3F)c3c(C#N)cnn3c2)cnn1C1CCN(C(=O)[C@@H](C)N(C)C)CC1 ; Cc1c(-c2cc(Sc3ncccc3F)c3c(C#N)cnn3c2)cnn1C1CCN(C(=O)[C@H](C)N(C)C)CC1]

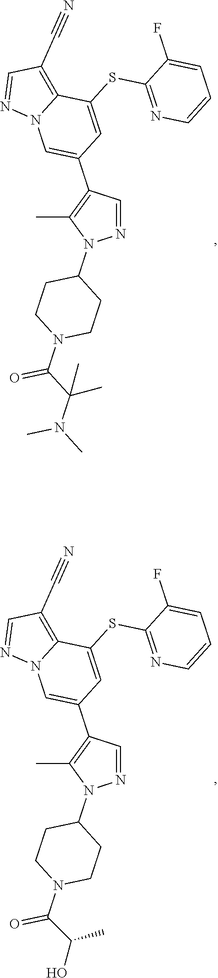 [SMILES: Cc1c(-c2cc(Sc3ncccc3F)c3c(C#N)cnn3c2)cnn1C1CCN(C(=O)C(C)(C)N(C)C)CC1 ; Cc1c(-c2cc(Sc3ncccc3F)c3c(C#N)cnn3c2)cnn1C1CCN(C(=O)[C@H](C)O)CC1]

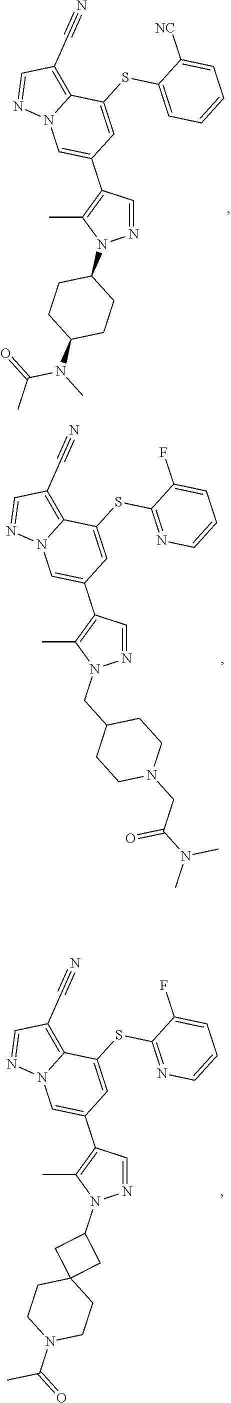

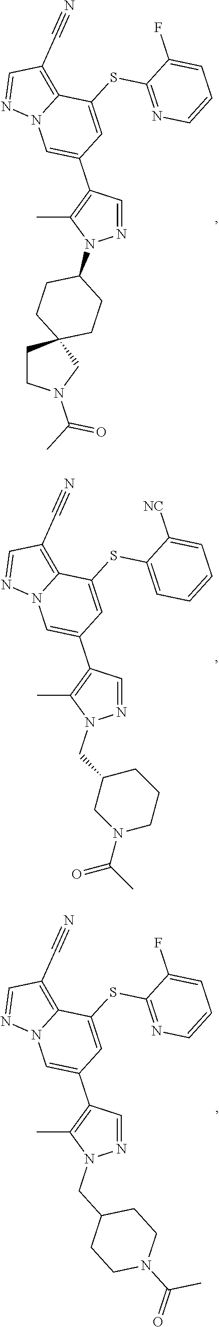

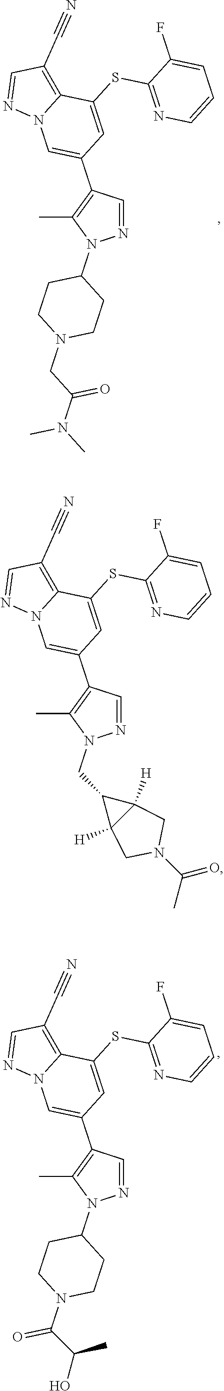 [SMILES: Cc1c(-c2cc(Sc3ncccc3F)c3c(C#N)cnn3c2)cnn1C1CCN(CC(=O)N(C)C)CC1 ; CC(=O)N1C[C@@H]2[C@H](C1)[C@H]2Cn1ncc(-c2cc(Sc3ncccc3F)c3c(C#N)cnn3c2)c1C ; Cc1c(-c2cc(Sc3ncccc3F)c3c(C#N)cnn3c2)cnn1C1CCN(C(=O)[C@@H](C)O)CC1]

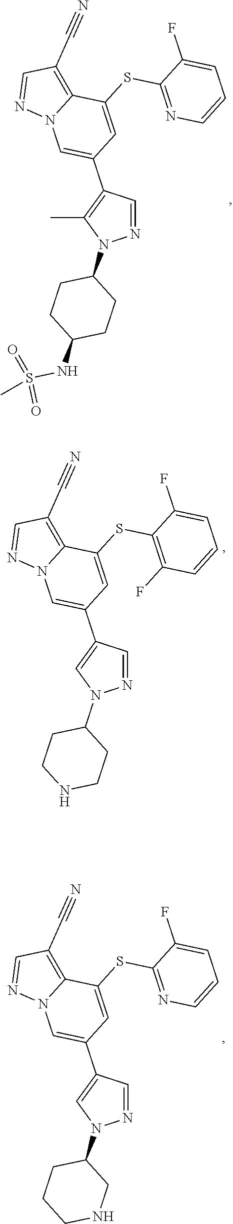 [SMILES: Cc1c(-c2cc(Sc3ncccc3F)c3c(C#N)cnn3c2)cnn1[C@H]1CC[C@@H](NS(C)(=O)=O)CC1 ; N#Cc1cnn2cc(-c3cnn(C4CCNCC4)c3)cc(Sc3c(F)cccc3F)c12 ; N#Cc1cnn2cc(-c3cnn([C@@H]4CCCNC4)c3)cc(Sc3ncccc3F)c12]

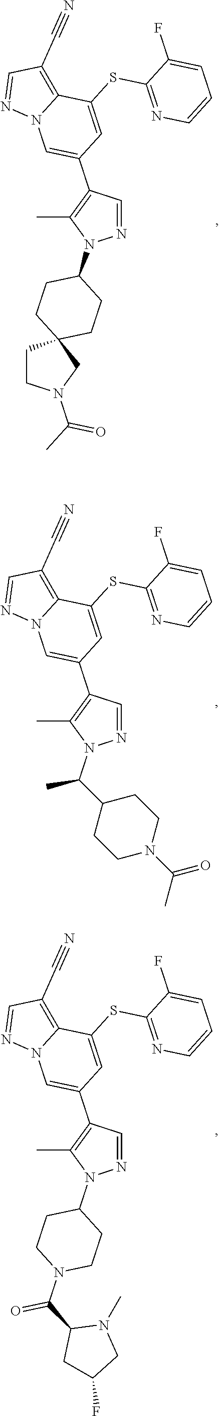 [SMILES: CC(=O)N1CC[C@]2(CC[C@H](n3ncc(-c4cc(Sc5ncccc5F)c5c(C#N)cnn5c4)c3C)CC2)C1 ; CC(=O)N1CCC([C@@H](C)n2ncc(-c3cc(Sc4ncccc4F)c4c(C#N)cnn4c3)c2C)CC1 ; Cc1c(-c2cc(Sc3ncccc3F)c3c(C#N)cnn3c2)cnn1C1CCN(C(=O)[C@@H]2C[C@@H](F)CN2C)CC1]

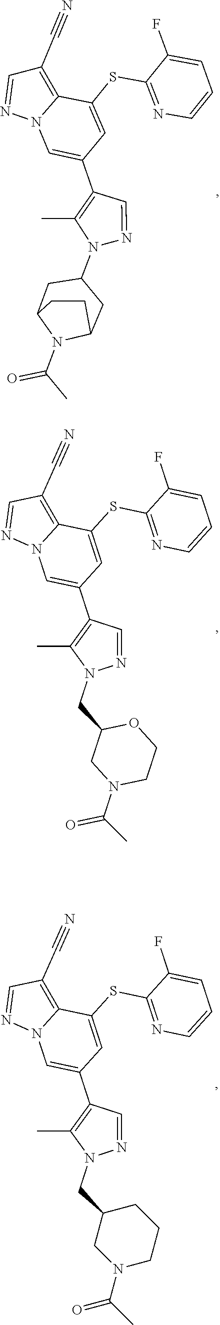

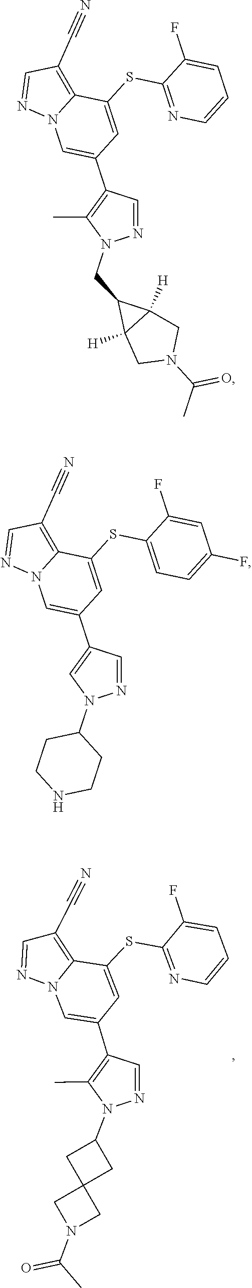 [SMILES: CC(=O)N1C[C@@H]2[C@H](C1)[C@@H]2Cn1ncc(-c2cc(Sc3ncccc3F)c3c(C#N)cnn3c2)c1C ; N#Cc1cnn2cc(-c3cnn(C4CCNCC4)c3)cc(Sc3ccc(F)cc3F)c12 ; CC(=O)N1CC2(CC(n3ncc(-c4cc(Sc5ncccc5F)c5c(C#N)cnn5c4)c3C)C2)C1]

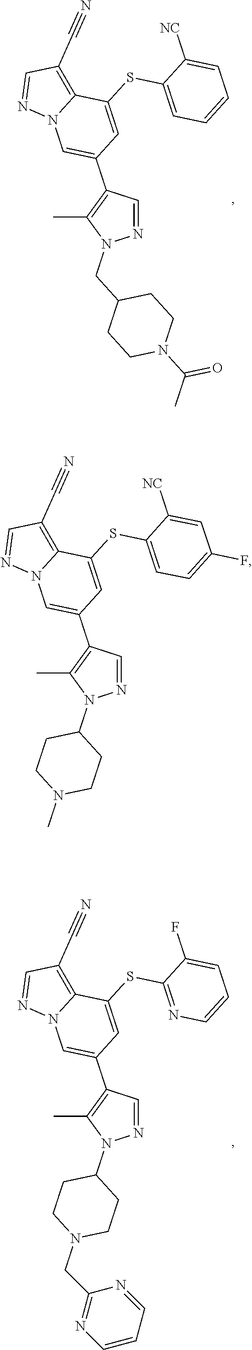 [SMILES: CC(=O)N1CCC(Cn2ncc(-c3cc(Sc4ccccc4C#N)c4c(C#N)cnn4c3)c2C)CC1 ; Cc1c(-c2cc(Sc3ccc(F)cc3C#N)c3c(C#N)cnn3c2)cnn1C1CCN(C)CC1 ; Cc1c(-c2cc(Sc3ncccc3F)c3c(C#N)cnn3c2)cnn1C1CCN(Cc2ncccn2)CC1]

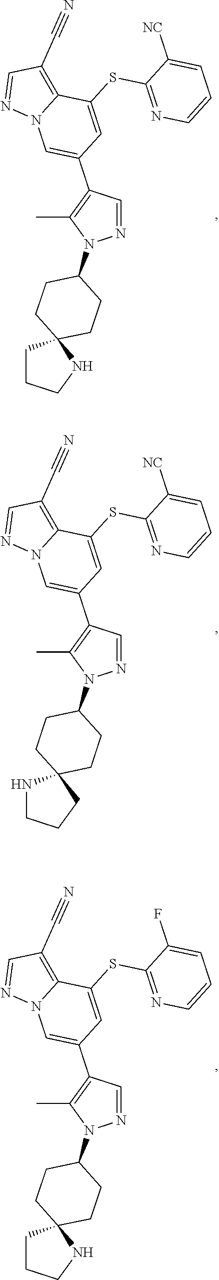 [SMILES: Cc1c(-c2cc(Sc3ncccc3C#N)c3c(C#N)cnn3c2)cnn1[C@H]1CC[C@]2(CCCN2)CC1 ; Cc1c(-c2cc(Sc3ncccc3C#N)c3c(C#N)cnn3c2)cnn1[C@H]1CC[C@@]2(CCCN2)CC1 ; Cc1c(-c2cc(Sc3ncccc3F)c3c(C#N)cnn3c2)cnn1[C@H]1CC[C@]2(CCCN2)CC1]

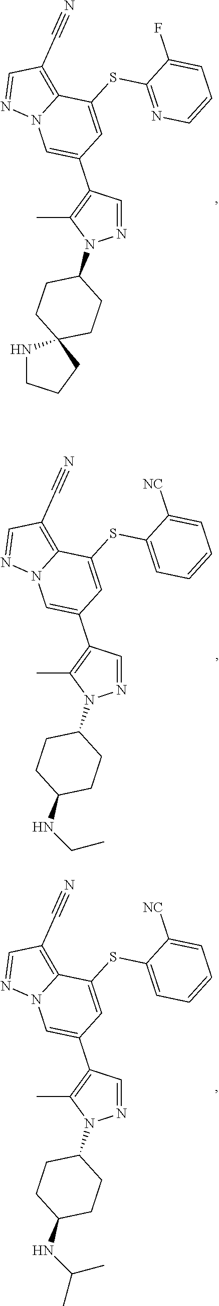

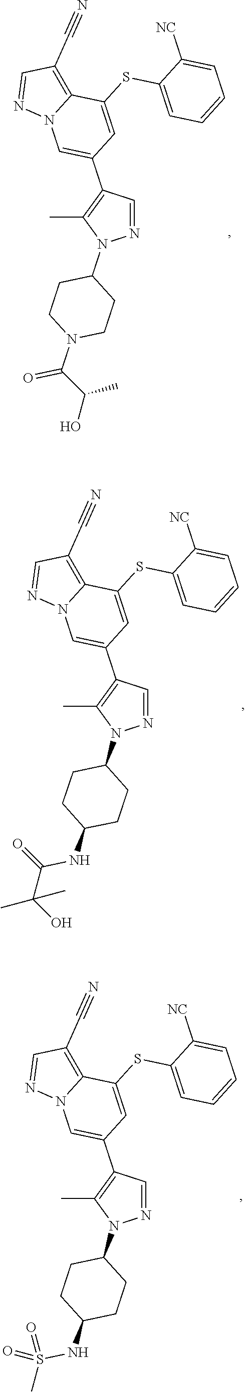 [SMILES: Cc1c(-c2cc(Sc3ccccc3C#N)c3c(C#N)cnn3c2)cnn1C1CCN(C(=O)[C@H](C)O)CC1 ; Cc1c(-c2cc(Sc3ccccc3C#N)c3c(C#N)cnn3c2)cnn1[C@H]1CC[C@@H](NC(=O)C(C)(C)O)CC1 ; Cc1c(-c2cc(Sc3ccccc3C#N)c3c(C#N)cnn3c2)cnn1[C@H]1CC[C@@H](NS(C)(=O)=O)CC1]

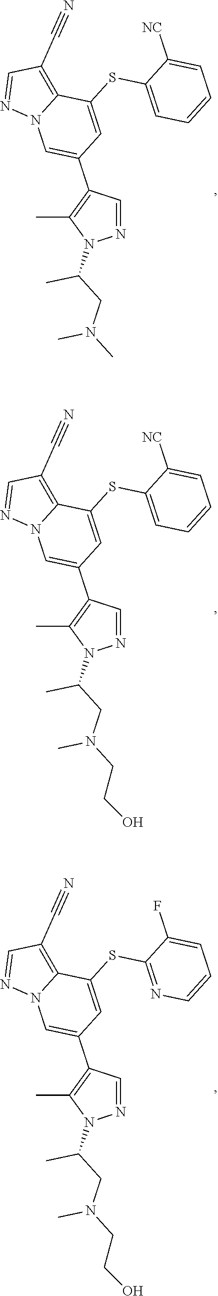 [SMILES: Cc1c(-c2cc(Sc3ccccc3C#N)c3c(C#N)cnn3c2)cnn1[C@@H](C)CN(C)C ; Cc1c(-c2cc(Sc3ccccc3C#N)c3c(C#N)cnn3c2)cnn1[C@@H](C)CN(C)CCO ; Cc1c(-c2cc(Sc3ncccc3F)c3c(C#N)cnn3c2)cnn1[C@@H](C)CN(C)CCO]

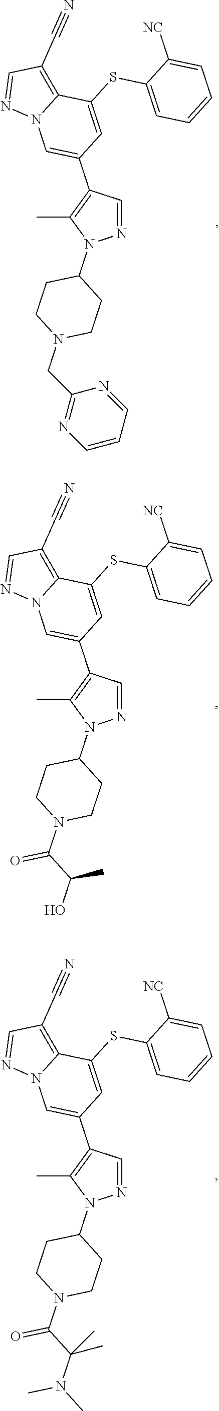

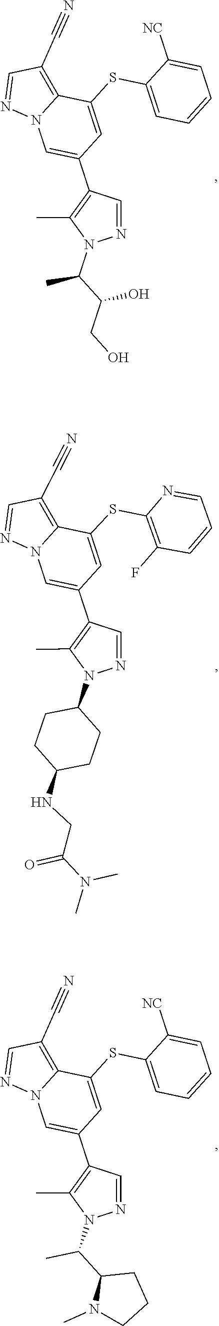 [SMILES: Cc1c(-c2cc(Sc3ccccc3C#N)c3c(C#N)cnn3c2)cnn1[C@H](C)[C@H](O)CO ; Cc1c(-c2cc(Sc3ncccc3F)c3c(C#N)cnn3c2)cnn1[C@H]1CC[C@@H](NCC(=O)N(C)C)CC1 ; Cc1c(-c2cc(Sc3ccccc3C#N)c3c(C#N)cnn3c2)cnn1[C@@H](C)[C@H]1CCCN1C]

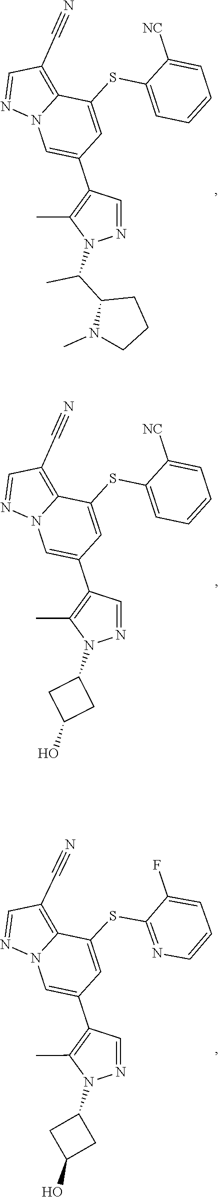 [SMILES: Cc1c(-c2cc(Sc3ccccc3C#N)c3c(C#N)cnn3c2)cnn1[C@@H](C)[C@@H]1CCCN1C ; Cc1c(-c2cc(Sc3ccccc3C#N)c3c(C#N)cnn3c2)cnn1[C@H]1C[C@@H](O)C1 ; Cc1c(-c2cc(Sc3ncccc3F)c3c(C#N)cnn3c2)cnn1[C@H]1C[C@H](O)C1]

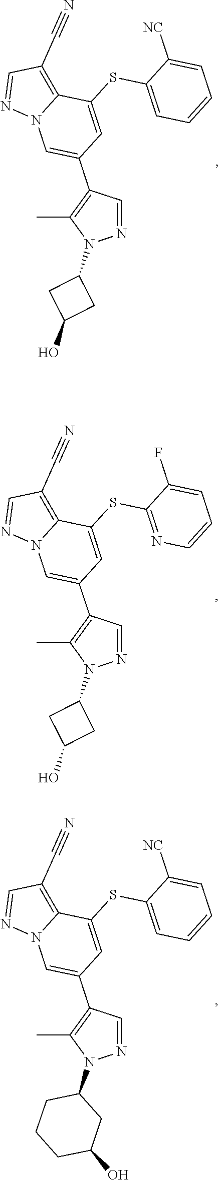 [SMILES: Cc1c(-c2cc(Sc3ccccc3C#N)c3c(C#N)cnn3c2)cnn1[C@H]1C[C@H](O)C1 ; Cc1c(-c2cc(Sc3ncccc3F)c3c(C#N)cnn3c2)cnn1[C@H]1C[C@@H](O)C1 ; Cc1c(-c2cc(Sc3ccccc3C#N)c3c(C#N)cnn3c2)cnn1[C@@H]1CCC[C@H](O)C1]

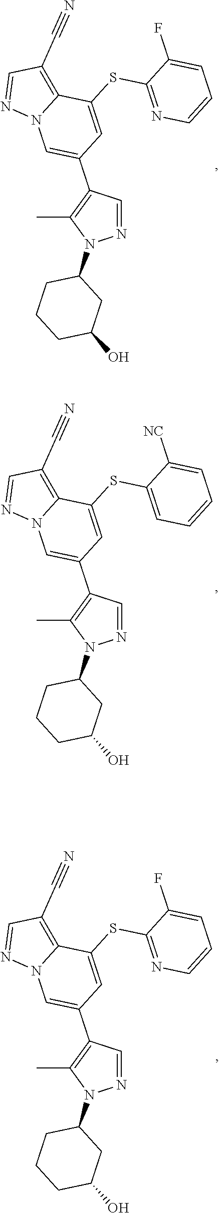 [SMILES: Cc1c(-c2cc(Sc3ncccc3F)c3c(C#N)cnn3c2)cnn1[C@@H]1CCC[C@H](O)C1 ; Cc1c(-c2cc(Sc3ccccc3C#N)c3c(C#N)cnn3c2)cnn1[C@@H]1CCC[C@@H](O)C1 ; Cc1c(-c2cc(Sc3ncccc3F)c3c(C#N)cnn3c2)cnn1[C@@H]1CCC[C@@H](O)C1]

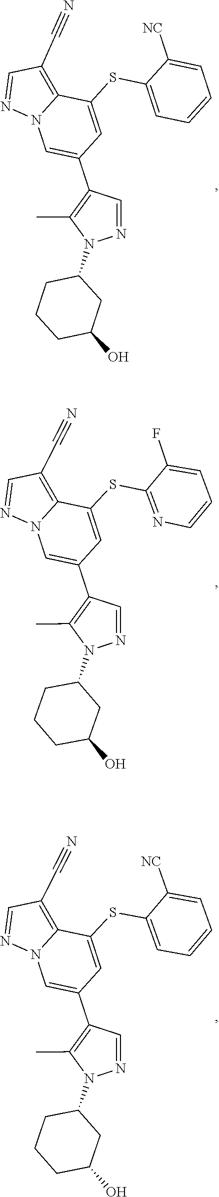 [SMILES: Cc1c(-c2cc(Sc3ccccc3C#N)c3c(C#N)cnn3c2)cnn1[C@H]1CCC[C@H](O)C1 ; Cc1c(-c2cc(Sc3ncccc3F)c3c(C#N)cnn3c2)cnn1[C@H]1CCC[C@H](O)C1 ; Cc1c(-c2cc(Sc3ccccc3C#N)c3c(C#N)cnn3c2)cnn1[C@H]1CCC[C@@H](O)C1]

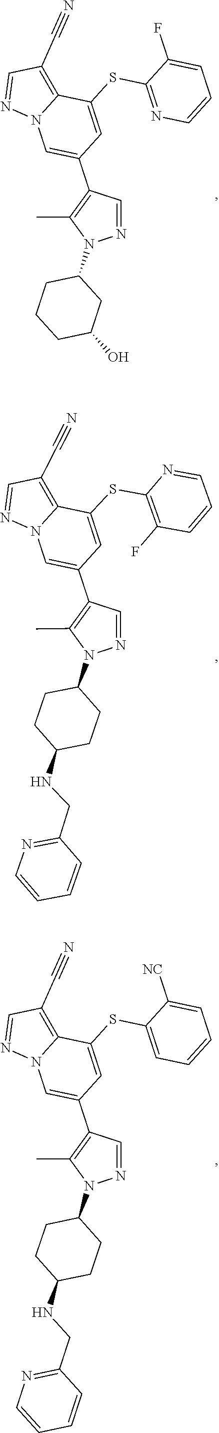 [SMILES: Cc1c(-c2cc(Sc3ncccc3F)c3c(C#N)cnn3c2)cnn1[C@H]1CCC[C@@H](O)C1 ; Cc1c(-c2cc(Sc3ncccc3F)c3c(C#N)cnn3c2)cnn1[C@H]1CC[C@@H](NCc2ccccn2)CC1 ; Cc1c(-c2cc(Sc3ccccc3C#N)c3c(C#N)cnn3c2)cnn1[C@H]1CC[C@@H](NCc2ccccn2)CC1]

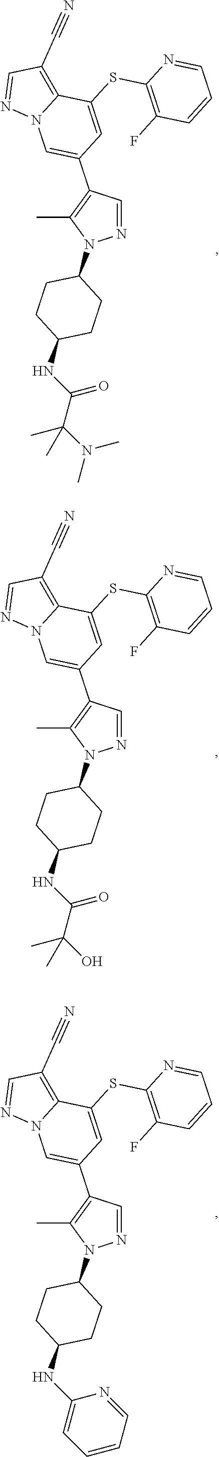

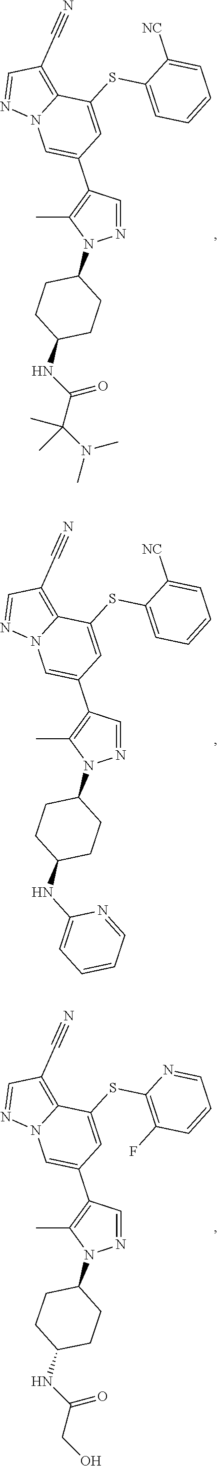

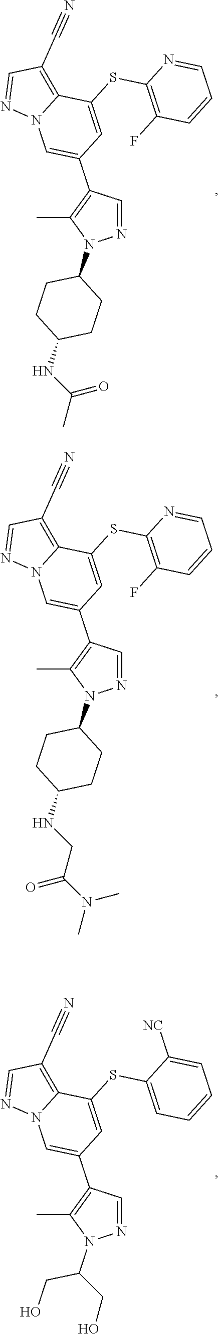

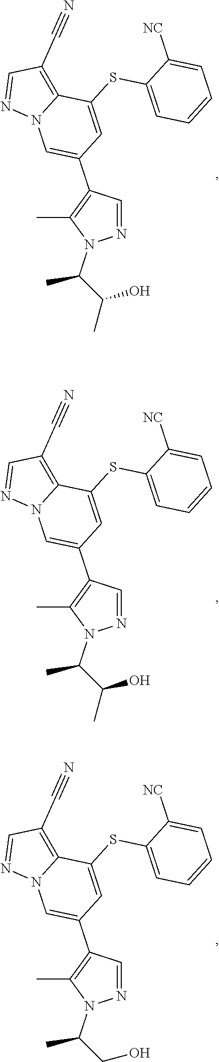 [SMILES: Cc1c(-c2cc(Sc3ccccc3C#N)c3c(C#N)cnn3c2)cnn1[C@H](C)[C@@H](C)O ; Cc1c(-c2cc(Sc3ccccc3C#N)c3c(C#N)cnn3c2)cnn1[C@H](C)[C@H](C)O ; Cc1c(-c2cc(Sc3ccccc3C#N)c3c(C#N)cnn3c2)cnn1[C@H](C)CO]

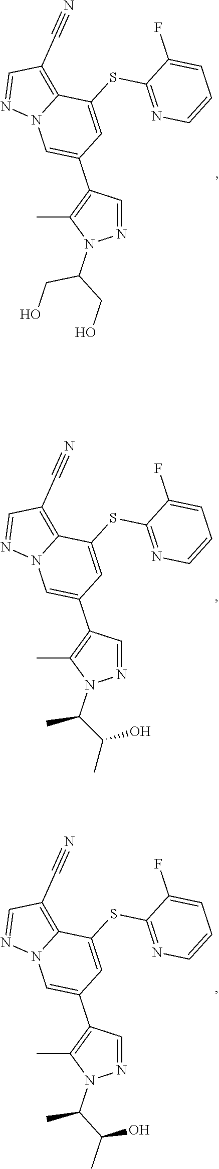 [SMILES: Cc1c(-c2cc(Sc3ncccc3F)c3c(C#N)cnn3c2)cnn1C(CO)CO ; Cc1c(-c2cc(Sc3ncccc3F)c3c(C#N)cnn3c2)cnn1[C@H](C)[C@@H](C)O ; Cc1c(-c2cc(Sc3ncccc3F)c3c(C#N)cnn3c2)cnn1[C@H](C)[C@H](C)O]

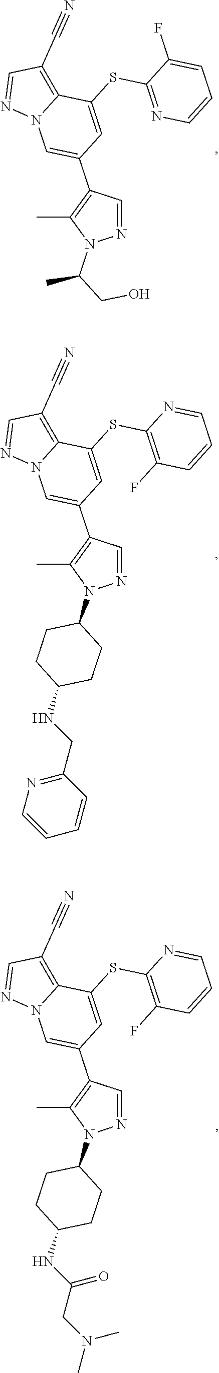 [SMILES: Cc1c(-c2cc(Sc3ncccc3F)c3c(C#N)cnn3c2)cnn1[C@H](C)CO ; Cc1c(-c2cc(Sc3ncccc3F)c3c(C#N)cnn3c2)cnn1[C@H]1CC[C@H](NCc2ccccn2)CC1 ; Cc1c(-c2cc(Sc3ncccc3F)c3c(C#N)cnn3c2)cnn1[C@H]1CC[C@H](NC(=O)CN(C)C)CC1]

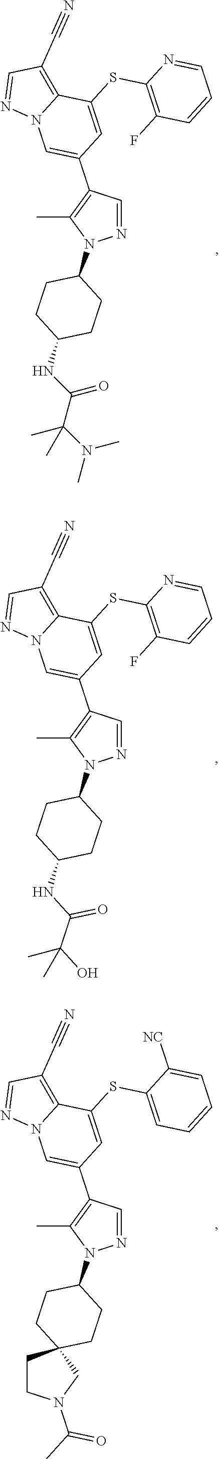

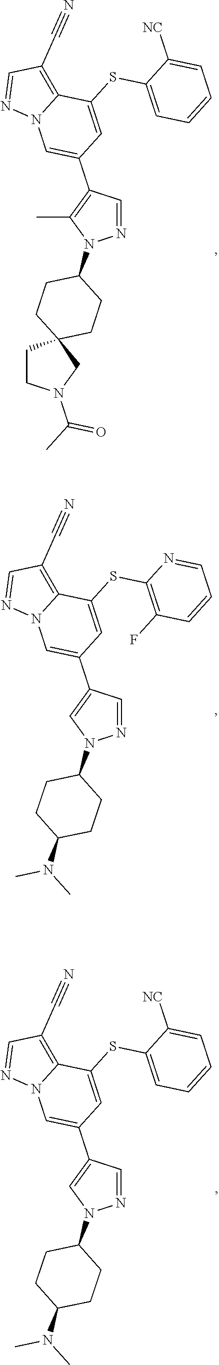 [SMILES: CC(=O)N1CC[C@]2(CC[C@H](n3ncc(-c4cc(Sc5ccccc5C#N)c5c(C#N)cnn5c4)c3C)CC2)C1 ; CN(C)[C@H]1CC[C@@H](n2cc(-c3cc(Sc4ncccc4F)c4c(C#N)cnn4c3)cn2)CC1 ; CN(C)[C@H]1CC[C@@H](n2cc(-c3cc(Sc4ccccc4C#N)c4c(C#N)cnn4c3)cn2)CC1]

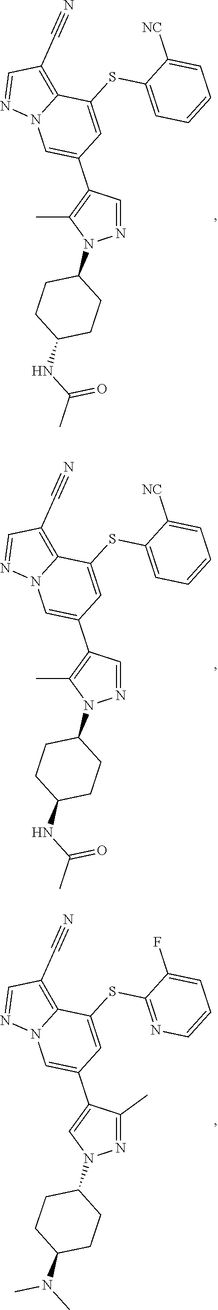

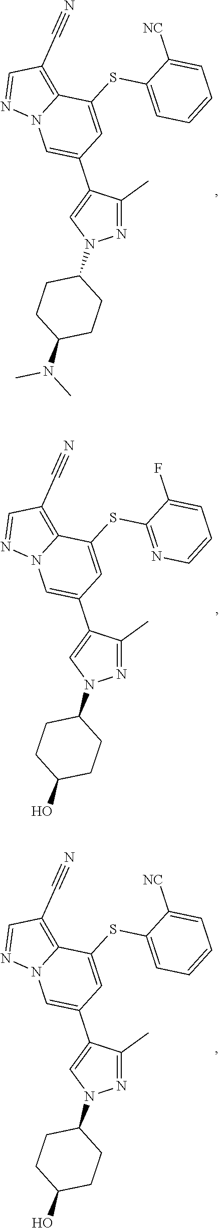 [SMILES: Cc1nn([C@H]2CC[C@H](N(C)C)CC2)cc1-c1cc(Sc2ccccc2C#N)c2c(C#N)cnn2c1 ; Cc1nn([C@H]2CC[C@@H](O)CC2)cc1-c1cc(Sc2ncccc2F)c2c(C#N)cnn2c1 ; Cc1nn([C@H]2CC[C@@H](O)CC2)cc1-c1cc(Sc2ccccc2C#N)c2c(C#N)cnn2c1]

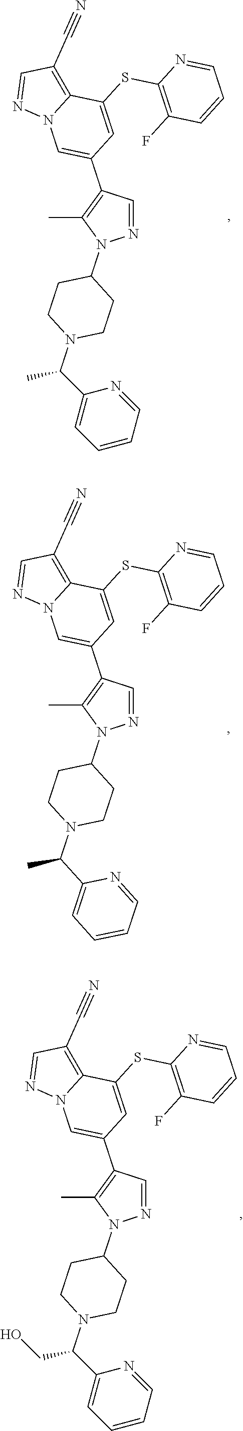

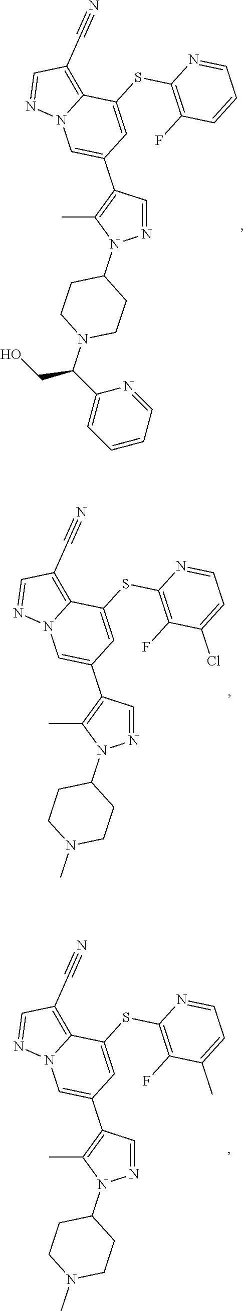 [SMILES: Cc1c(-c2cc(Sc3ncccc3F)c3c(C#N)cnn3c2)cnn1C1CCN([C@H](CO)c2ccccn2)CC1 ; Cc1c(-c2cc(Sc3nccc(Cl)c3F)c3c(C#N)cnn3c2)cnn1C1CCN(C)CC1 ; Cc1ccnc(Sc2cc(-c3cnn(C4CCN(C)CC4)c3C)cn3ncc(C#N)c23)c1F]

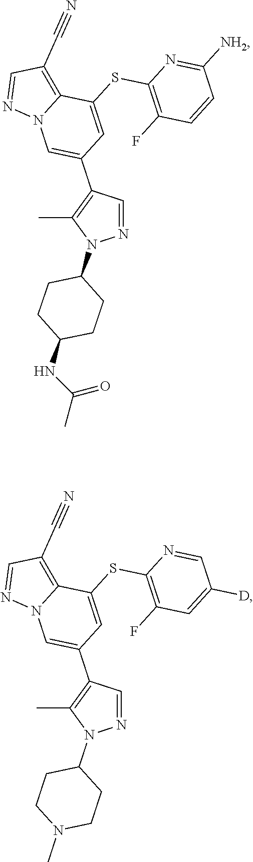

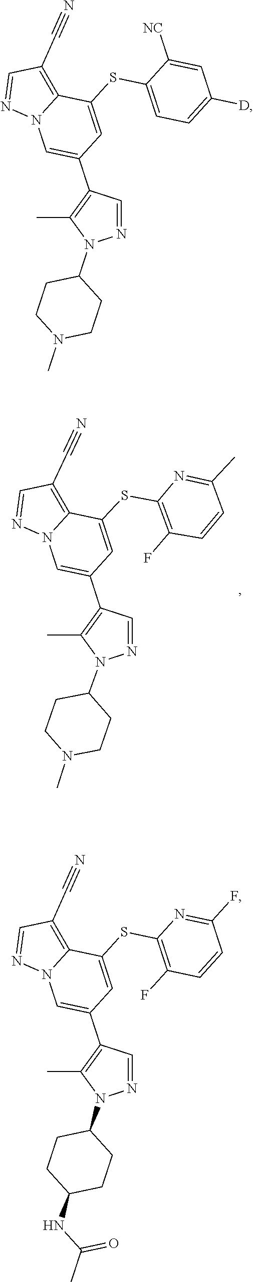

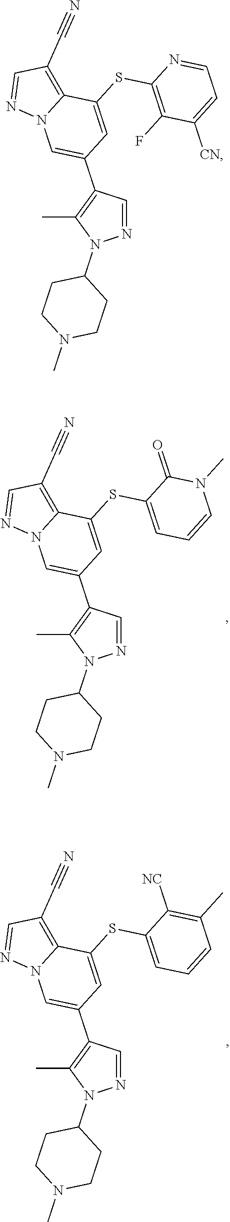 [SMILES: Cc1c(-c2cc(Sc3nccc(C#N)c3F)c3c(C#N)cnn3c2)cnn1C1CCN(C)CC1 ; Cc1c(-c2cc(Sc3cccn(C)c3=O)c3c(C#N)cnn3c2)cnn1C1CCN(C)CC1 ; Cc1cccc(Sc2cc(-c3cnn(C4CCN(C)CC4)c3C)cn3ncc(C#N)c23)c1C#N]

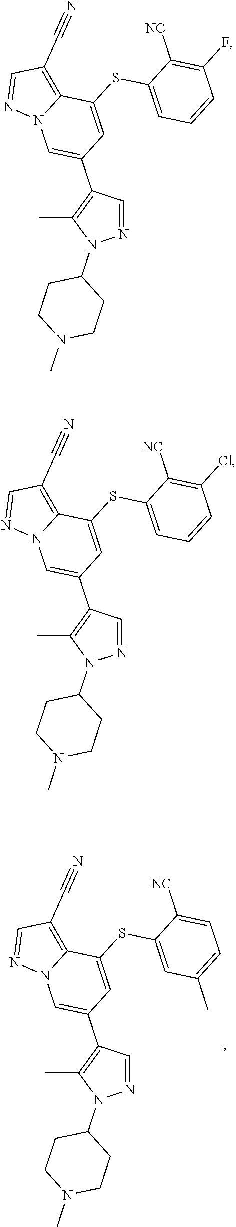 [SMILES: Cc1c(-c2cc(Sc3cccc(F)c3C#N)c3c(C#N)cnn3c2)cnn1C1CCN(C)CC1 ; Cc1c(-c2cc(Sc3cccc(Cl)c3C#N)c3c(C#N)cnn3c2)cnn1C1CCN(C)CC1 ; Cc1ccc(C#N)c(Sc2cc(-c3cnn(C4CCN(C)CC4)c3C)cn3ncc(C#N)c23)c1]

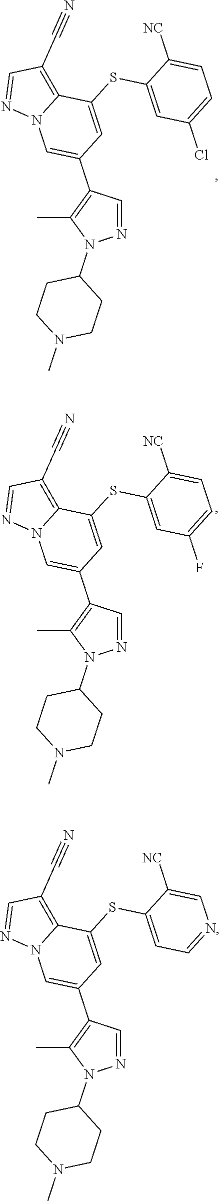 [SMILES: Cc1c(-c2cc(Sc3cc(Cl)ccc3C#N)c3c(C#N)cnn3c2)cnn1C1CCN(C)CC1 ; Cc1c(-c2cc(Sc3cc(F)ccc3C#N)c3c(C#N)cnn3c2)cnn1C1CCN(C)CC1 ; Cc1c(-c2cc(Sc3ccncc3C#N)c3c(C#N)cnn3c2)cnn1C1CCN(C)CC1]

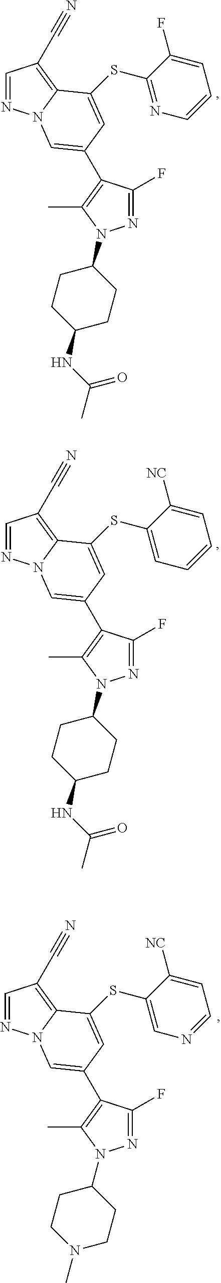

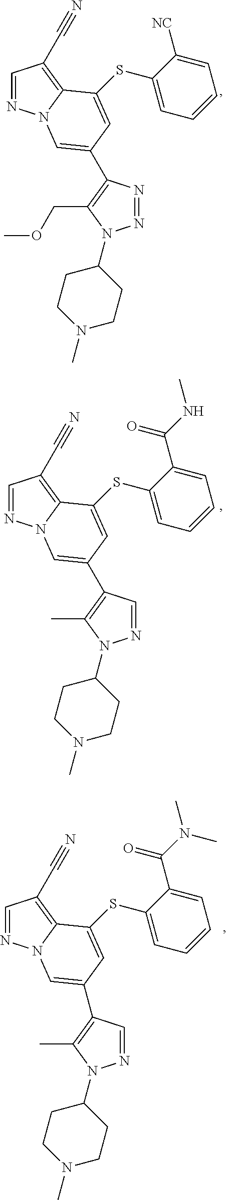 [SMILES: COCc1c(-c2cc(Sc3ccccc3C#N)c3c(C#N)cnn3c2)nnn1C1CCN(C)CC1 ; CNC(=O)c1ccccc1Sc1cc(-c2cnn(C3CCN(C)CC3)c2C)cn2ncc(C#N)c12 ; Cc1c(-c2cc(Sc3ccccc3C(=O)N(C)C)c3c(C#N)cnn3c2)cnn1C1CCN(C)CC1]

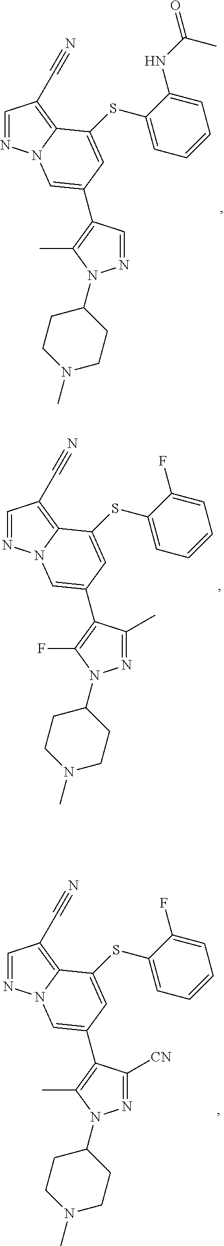

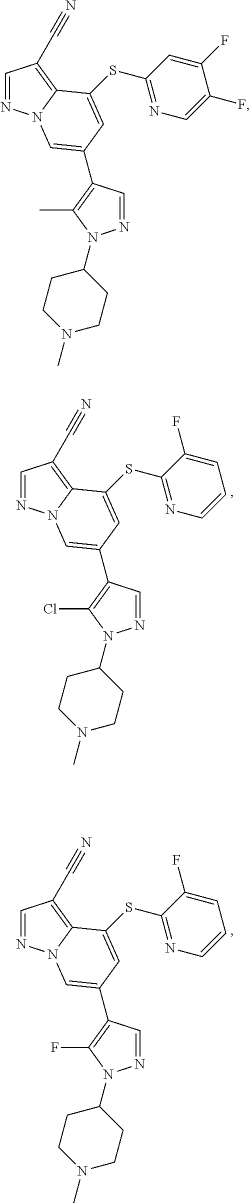 [SMILES: Cc1c(-c2cc(Sc3cc(F)c(F)cn3)c3c(C#N)cnn3c2)cnn1C1CCN(C)CC1 ; CN1CCC(n2ncc(-c3cc(Sc4ncccc4F)c4c(C#N)cnn4c3)c2Cl)CC1 ; CN1CCC(n2ncc(-c3cc(Sc4ncccc4F)c4c(C#N)cnn4c3)c2F)CC1]

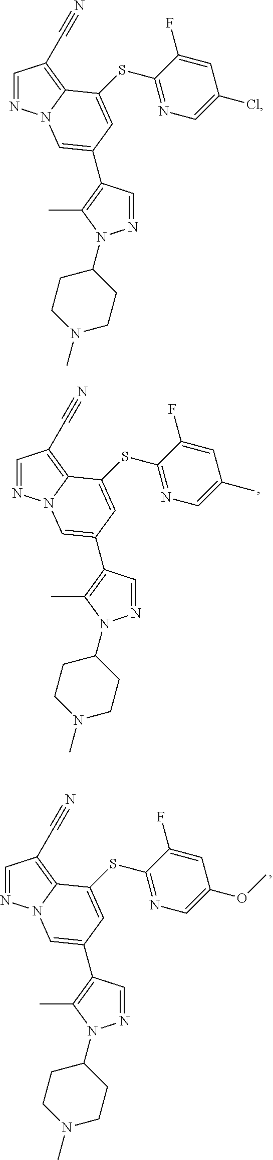

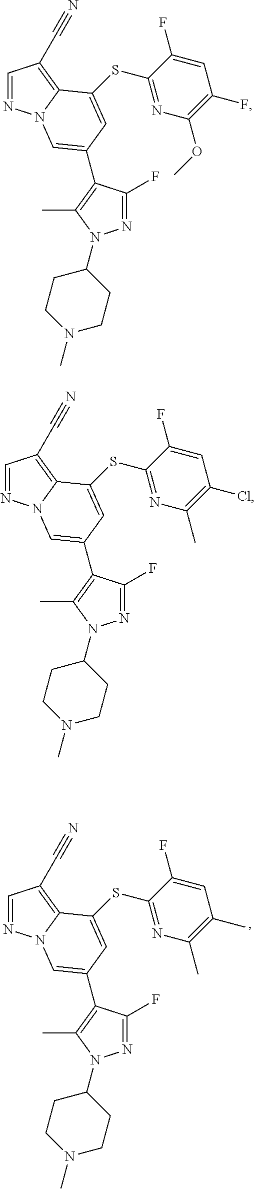

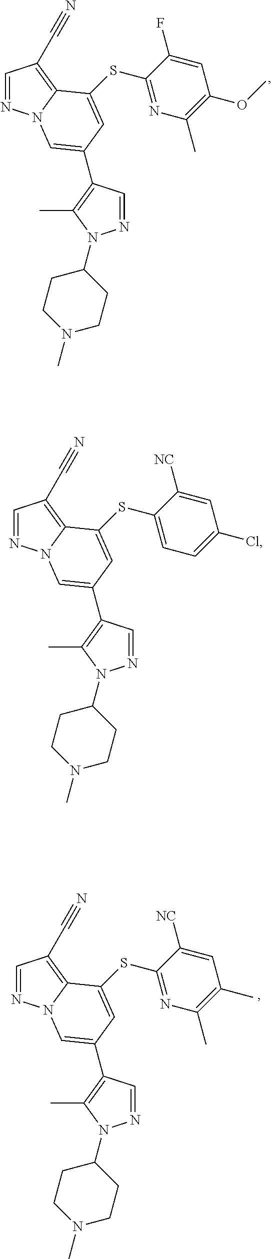 [SMILES: COc1cc(F)c(Sc2cc(-c3cnn(C4CCN(C)CC4)c3C)cn3ncc(C#N)c23)nc1C ; Cc1c(-c2cc(Sc3ccc(Cl)cc3C#N)c3c(C#N)cnn3c2)cnn1C1CCN(C)CC1 ; Cc1cc(C#N)c(Sc2cc(-c3cnn(C4CCN(C)CC4)c3C)cn3ncc(C#N)c23)nc1C]

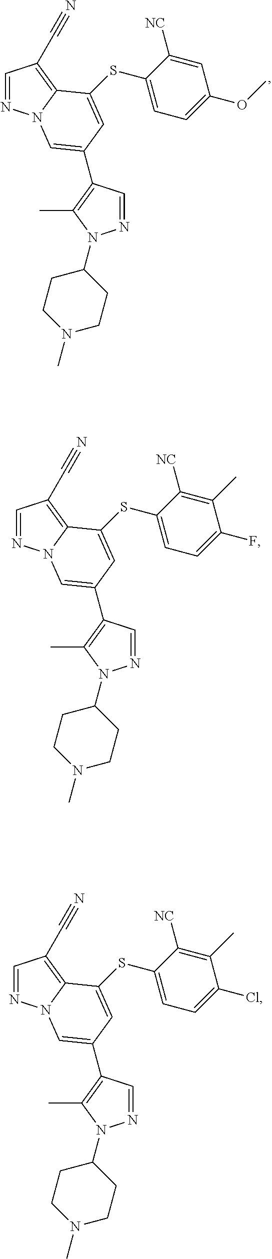 [SMILES: COc1ccc(Sc2cc(-c3cnn(C4CCN(C)CC4)c3C)cn3ncc(C#N)c23)c(C#N)c1 ; Cc1c(F)ccc(Sc2cc(-c3cnn(C4CCN(C)CC4)c3C)cn3ncc(C#N)c23)c1C#N ; Cc1c(Cl)ccc(Sc2cc(-c3cnn(C4CCN(C)CC4)c3C)cn3ncc(C#N)c23)c1C#N]

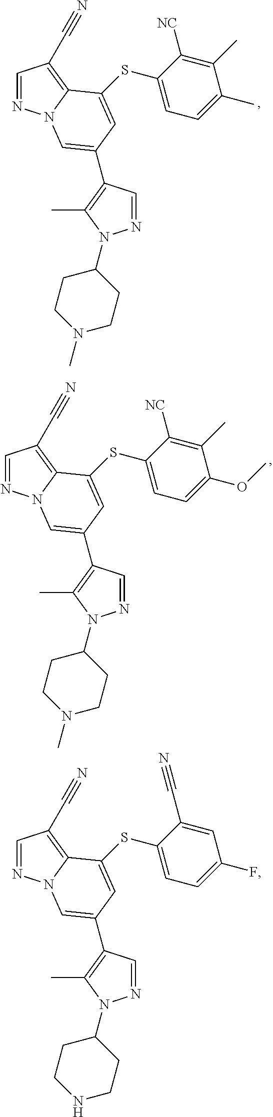

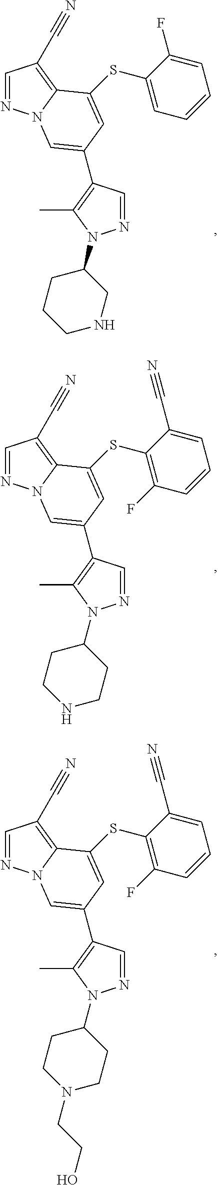

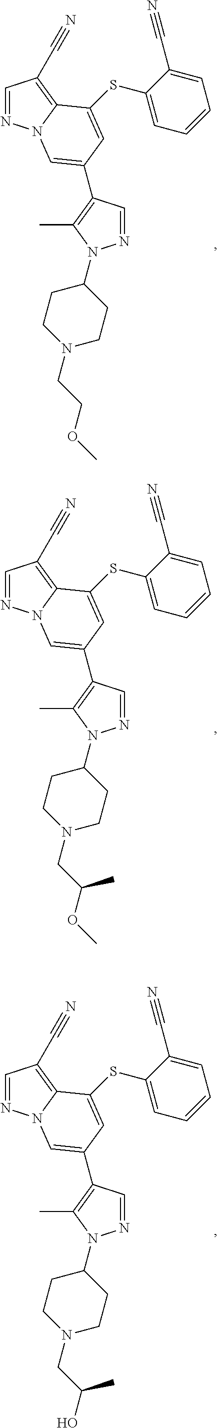 [SMILES: COCCN1CCC(n2ncc(-c3cc(Sc4ccccc4C#N)c4c(C#N)cnn4c3)c2C)CC1 ; CO[C@H](C)CN1CCC(n2ncc(-c3cc(Sc4ccccc4C#N)c4c(C#N)cnn4c3)c2C)CC1 ; Cc1c(-c2cc(Sc3ccccc3C#N)c3c(C#N)cnn3c2)cnn1C1CCN(C[C@@H](C)O)CC1]

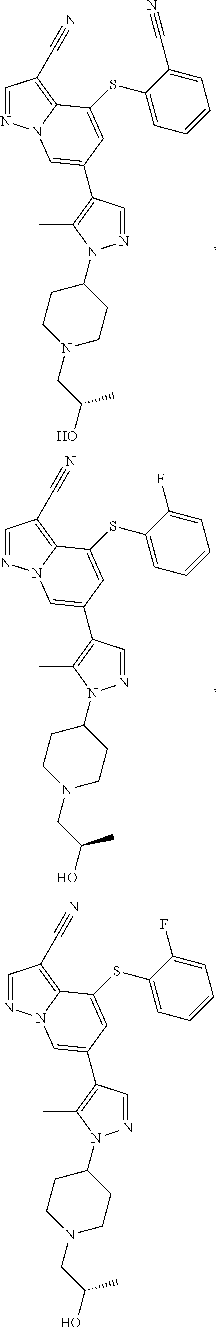

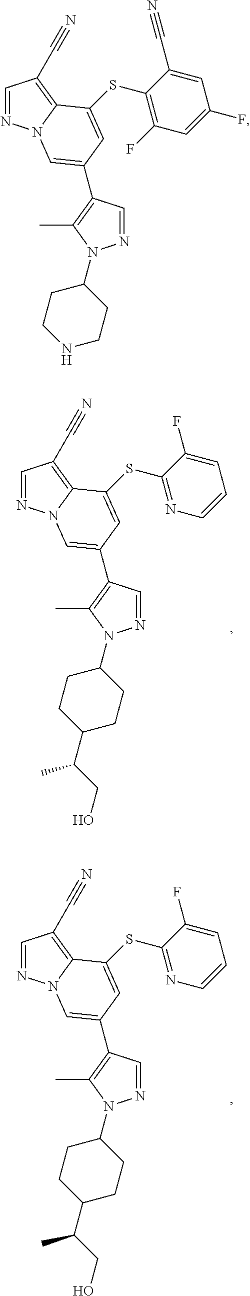 [SMILES: Cc1c(-c2cc(Sc3c(F)cc(F)cc3C#N)c3c(C#N)cnn3c2)cnn1C1CCNCC1 ; Cc1c(-c2cc(Sc3ncccc3F)c3c(C#N)cnn3c2)cnn1C1CCC([C@@H](C)CO)CC1 ; Cc1c(-c2cc(Sc3ncccc3F)c3c(C#N)cnn3c2)cnn1C1CCC([C@H](C)CO)CC1]

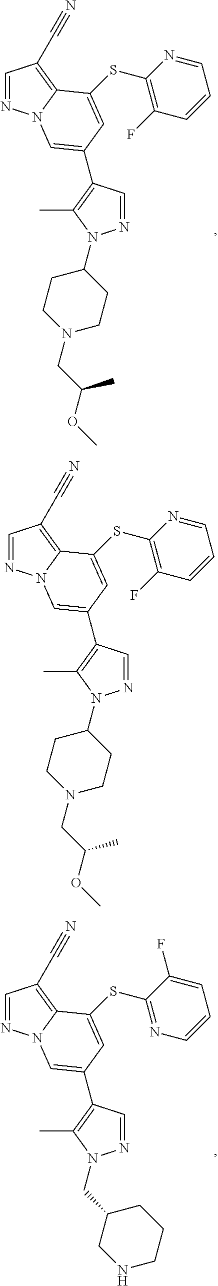

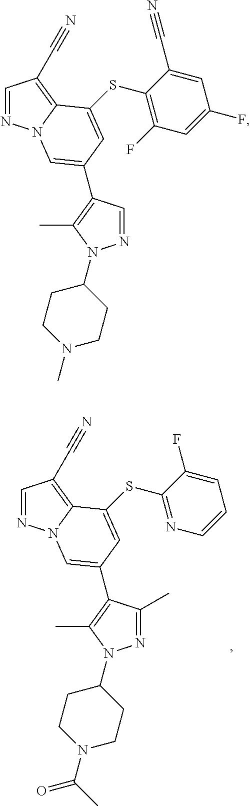 [SMILES: Cc1c(-c2cc(Sc3c(F)cc(F)cc3C#N)c3c(C#N)cnn3c2)cnn1C1CCN(C)CC1 ; CC(=O)N1CCC(n2nc(C)c(-c3cc(Sc4ncccc4F)c4c(C#N)cnn4c3)c2C)CC1]

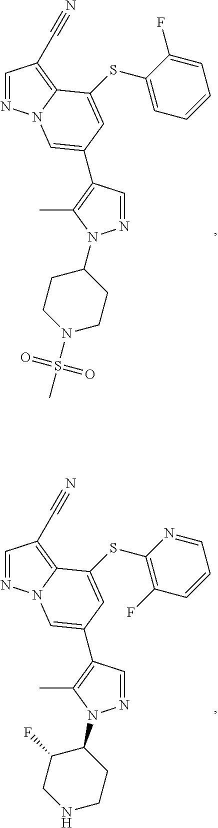 [SMILES: Cc1c(-c2cc(Sc3ccccc3F)c3c(C#N)cnn3c2)cnn1C1CCN(S(C)(=O)=O)CC1 ; Cc1c(-c2cc(Sc3ncccc3F)c3c(C#N)cnn3c2)cnn1[C@H]1CCNC[C@@H]1F]

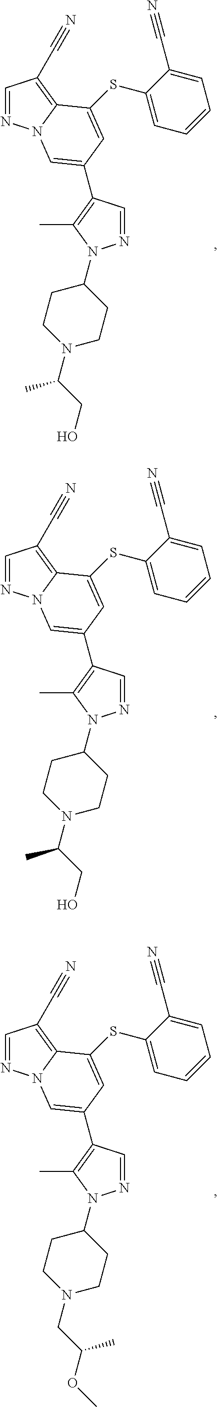 [SMILES: Cc1c(-c2cc(Sc3ccccc3C#N)c3c(C#N)cnn3c2)cnn1C1CCN([C@@H](C)CO)CC1 ; Cc1c(-c2cc(Sc3ccccc3C#N)c3c(C#N)cnn3c2)cnn1C1CCN([C@H](C)CO)CC1 ; CO[C@@H](C)CN1CCC(n2ncc(-c3cc(Sc4ccccc4C#N)c4c(C#N)cnn4c3)c2C)CC1]

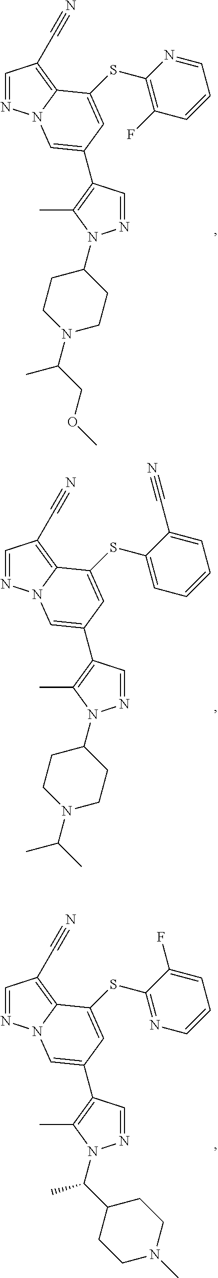

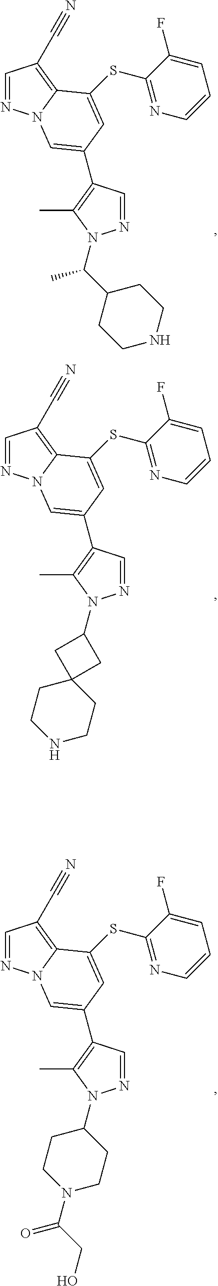 [SMILES: Cc1c(-c2cc(Sc3ncccc3F)c3c(C#N)cnn3c2)cnn1[C@@H](C)C1CCNCC1 ; Cc1c(-c2cc(Sc3ncccc3F)c3c(C#N)cnn3c2)cnn1C1CC2(CCNCC2)C1 ; Cc1c(-c2cc(Sc3ncccc3F)c3c(C#N)cnn3c2)cnn1C1CCN(C(=O)CO)CC1]

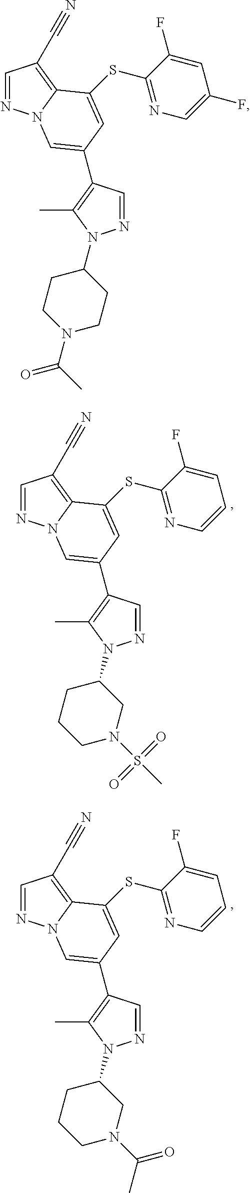

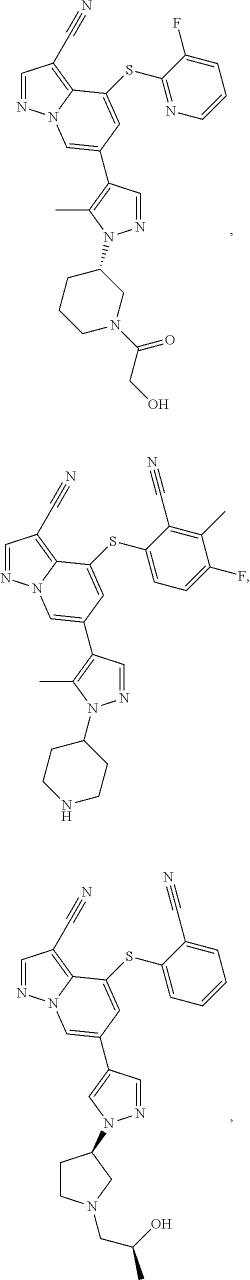 [SMILES: Cc1c(-c2cc(Sc3ncccc3F)c3c(C#N)cnn3c2)cnn1[C@H]1CCCN(C(=O)CO)C1 ; Cc1c(F)ccc(Sc2cc(-c3cnn(C4CCNCC4)c3C)cn3ncc(C#N)c23)c1C#N ; C[C@H](O)CN1CC[C@@H](n2cc(-c3cc(Sc4ccccc4C#N)c4c(C#N)cnn4c3)cn2)C1]

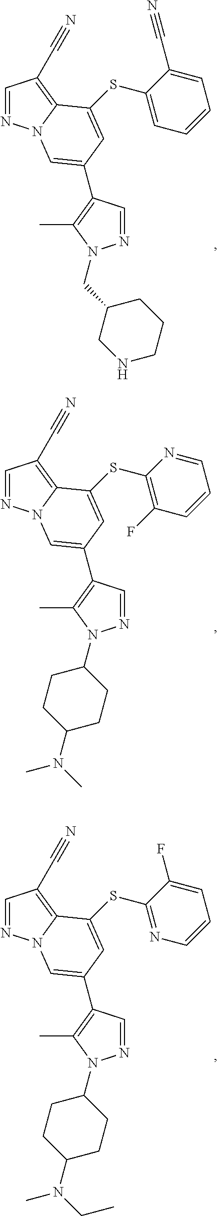

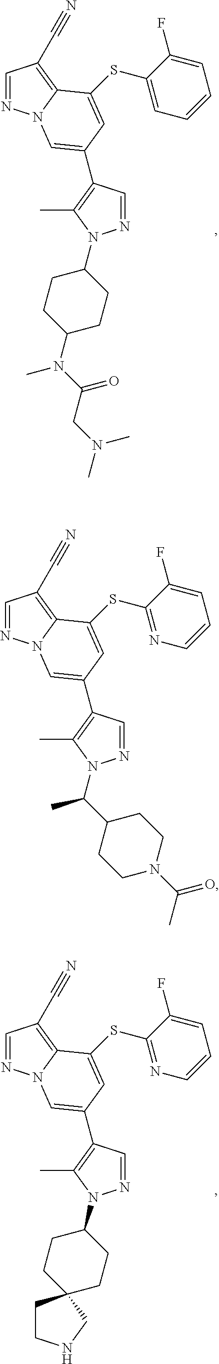 [SMILES: Cc1c(-c2cc(Sc3ccccc3F)c3c(C#N)cnn3c2)cnn1C1CCC(N(C)C(=O)CN(C)C)CC1 ; CC(=O)N1CCC([C@@H](C)n2ncc(-c3cc(Sc4ncccc4F)c4c(C#N)cnn4c3)c2C)CC1 ; Cc1c(-c2cc(Sc3ncccc3F)c3c(C#N)cnn3c2)cnn1[C@H]1CC[C@@]2(CCNC2)CC1]

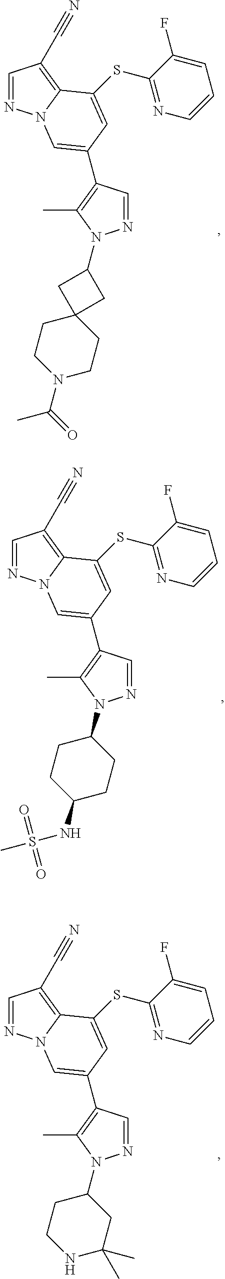

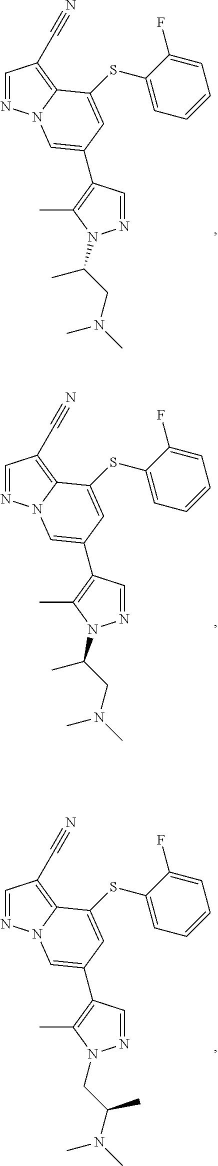 [SMILES: Cc1c(-c2cc(Sc3ccccc3F)c3c(C#N)cnn3c2)cnn1[C@@H](C)CN(C)C ; Cc1c(-c2cc(Sc3ccccc3F)c3c(C#N)cnn3c2)cnn1[C@H](C)CN(C)C ; Cc1c(-c2cc(Sc3ccccc3F)c3c(C#N)cnn3c2)cnn1C[C@@H](C)N(C)C]

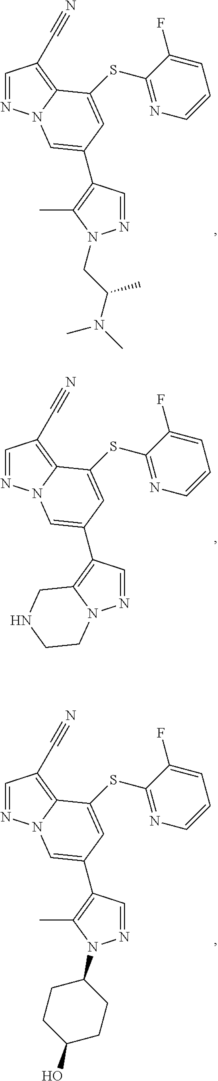 [SMILES: Cc1c(-c2cc(Sc3ncccc3F)c3c(C#N)cnn3c2)cnn1C[C@H](C)N(C)C ; N#Cc1cnn2cc(-c3cnn4c3CNCC4)cc(Sc3ncccc3F)c12 ; Cc1c(-c2cc(Sc3ncccc3F)c3c(C#N)cnn3c2)cnn1[C@H]1CC[C@@H](O)CC1]

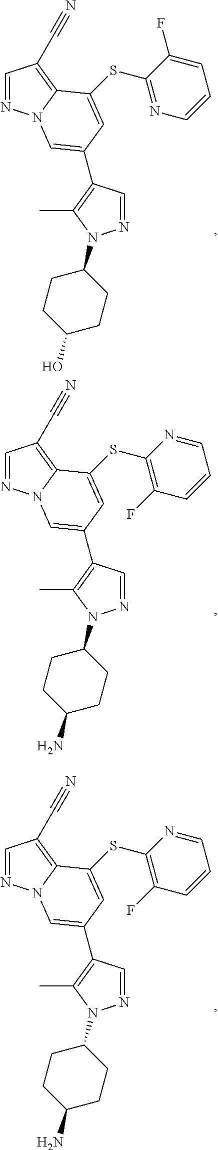

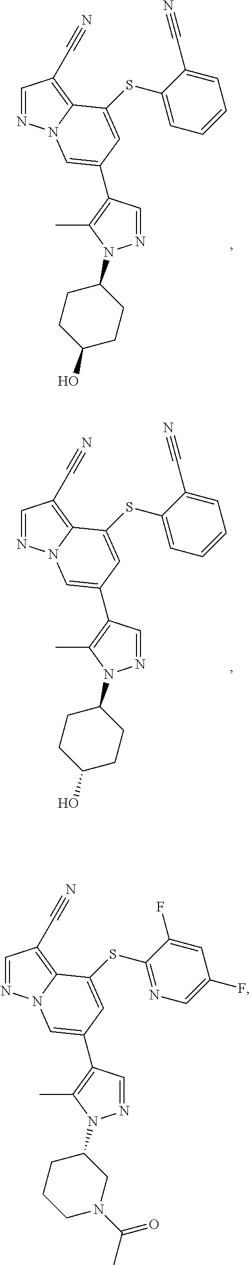 [SMILES: Cc1c(-c2cc(Sc3ccccc3C#N)c3c(C#N)cnn3c2)cnn1[C@H]1CC[C@@H](O)CC1 ; Cc1c(-c2cc(Sc3ccccc3C#N)c3c(C#N)cnn3c2)cnn1[C@H]1CC[C@H](O)CC1 ; CC(=O)N1CCC[C@H](n2ncc(-c3cc(Sc4ncc(F)cc4F)c4c(C#N)cnn4c3)c2C)C1]

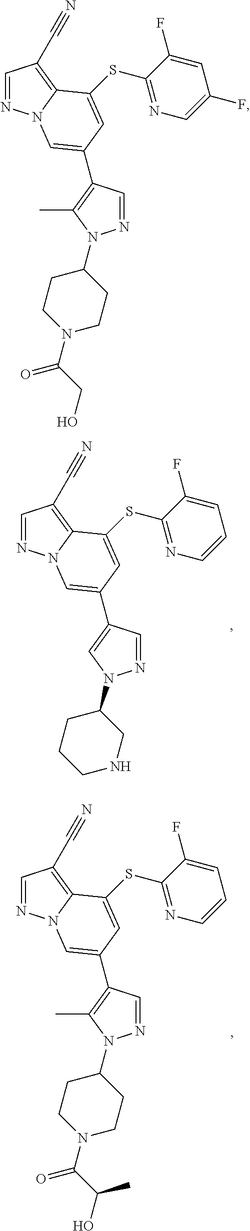

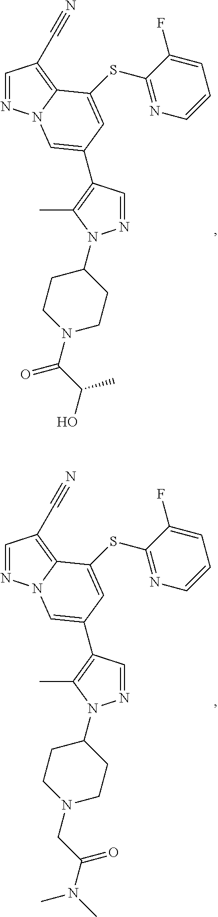 [SMILES: Cc1c(-c2cc(Sc3ncccc3F)c3c(C#N)cnn3c2)cnn1C1CCN(C(=O)[C@H](C)O)CC1 ; Cc1c(-c2cc(Sc3ncccc3F)c3c(C#N)cnn3c2)cnn1C1CCN(CC(=O)N(C)C)CC1]

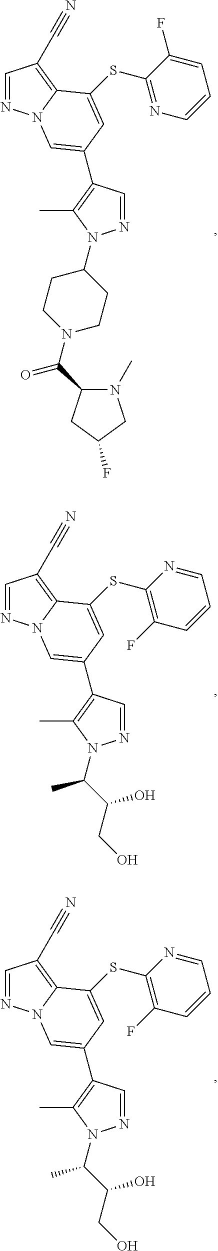 [SMILES: Cc1c(-c2cc(Sc3ncccc3F)c3c(C#N)cnn3c2)cnn1C1CCN(C(=O)[C@@H]2C[C@@H](F)CN2C)CC1 ; Cc1c(-c2cc(Sc3ncccc3F)c3c(C#N)cnn3c2)cnn1[C@H](C)[C@H](O)CO ; Cc1c(-c2cc(Sc3ncccc3F)c3c(C#N)cnn3c2)cnn1[C@@H](C)[C@H](O)CO]

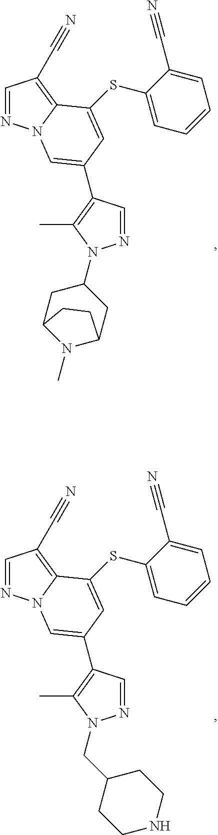 [SMILES: Cc1c(-c2cc(Sc3ccccc3C#N)c3c(C#N)cnn3c2)cnn1C1CC2CCC(C1)N2C ; Cc1c(-c2cc(Sc3ccccc3C#N)c3c(C#N)cnn3c2)cnn1CC1CCNCC1]

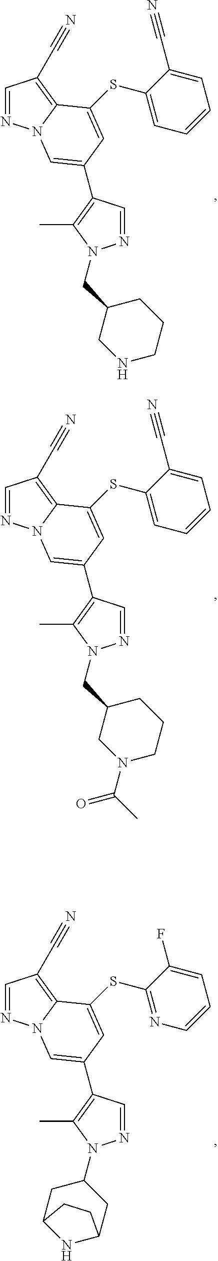 [SMILES: Cc1c(-c2cc(Sc3ccccc3C#N)c3c(C#N)cnn3c2)cnn1C[C@H]1CCCNC1 ; CC(=O)N1CCC[C@H](Cn2ncc(-c3cc(Sc4ccccc4C#N)c4c(C#N)cnn4c3)c2C)C1 ; Cc1c(-c2cc(Sc3ncccc3F)c3c(C#N)cnn3c2)cnn1C1CC2CCC(C1)N2]

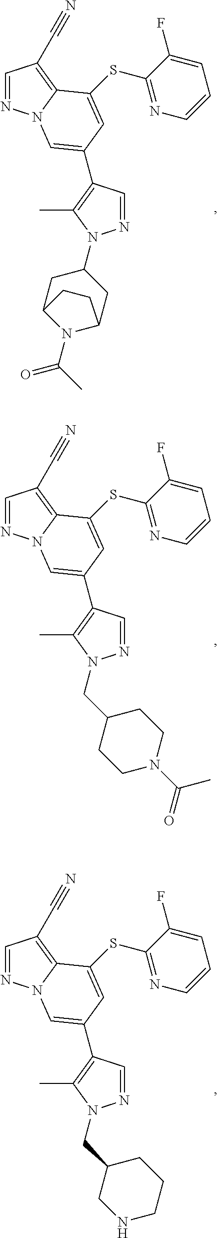

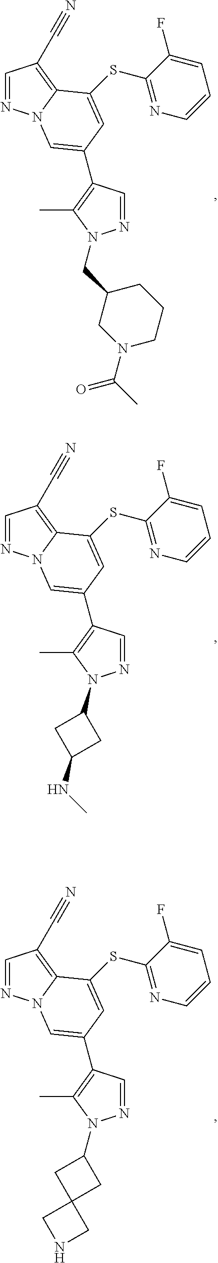

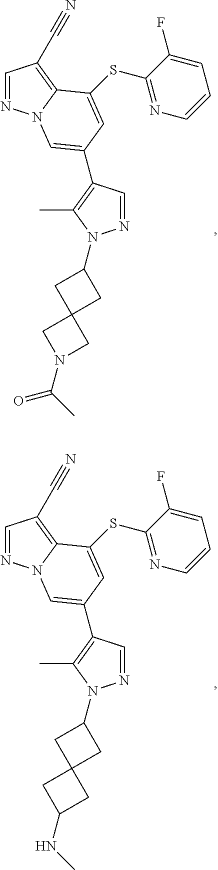

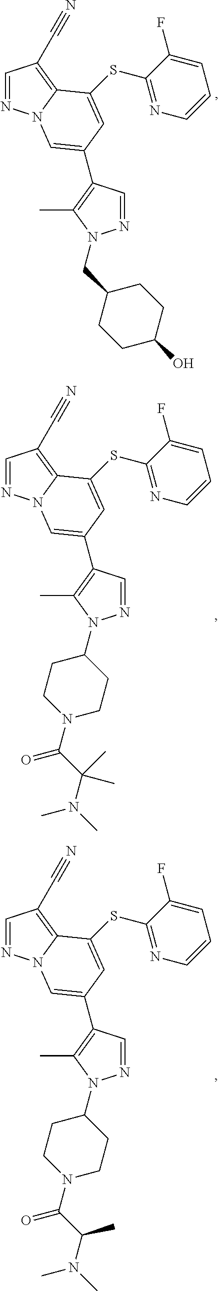

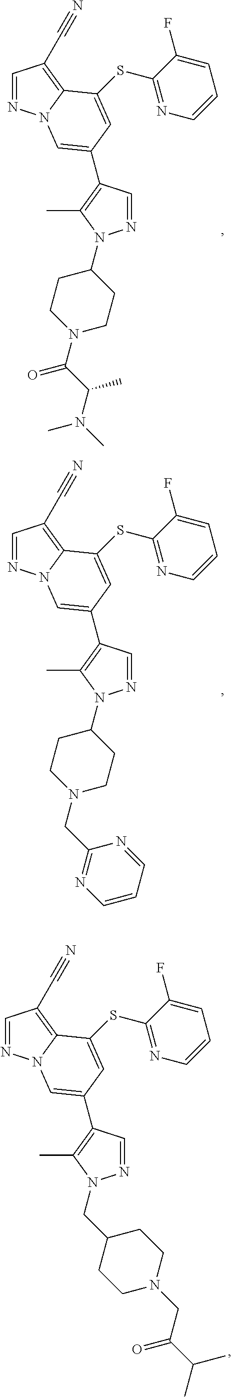

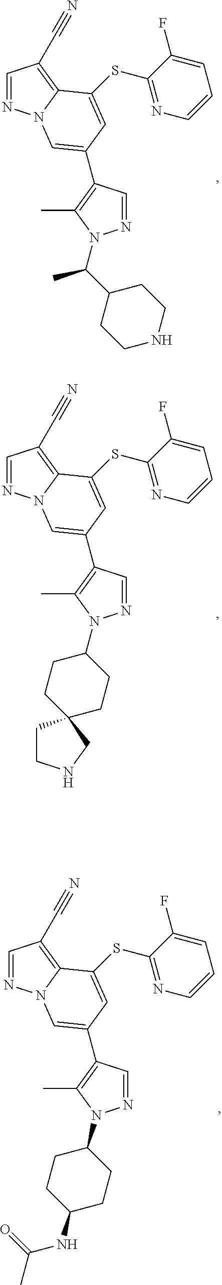

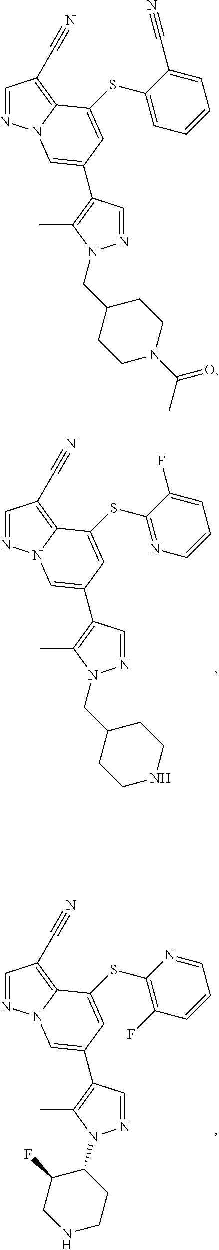 [SMILES: CC(=O)N1CCC(Cn2ncc(-c3cc(Sc4ccccc4C#N)c4c(C#N)cnn4c3)c2C)CC1 ; Cc1c(-c2cc(Sc3ncccc3F)c3c(C#N)cnn3c2)cnn1CC1CCNCC1 ; Cc1c(-c2cc(Sc3ncccc3F)c3c(C#N)cnn3c2)cnn1[C@@H]1CCNC[C@H]1F]

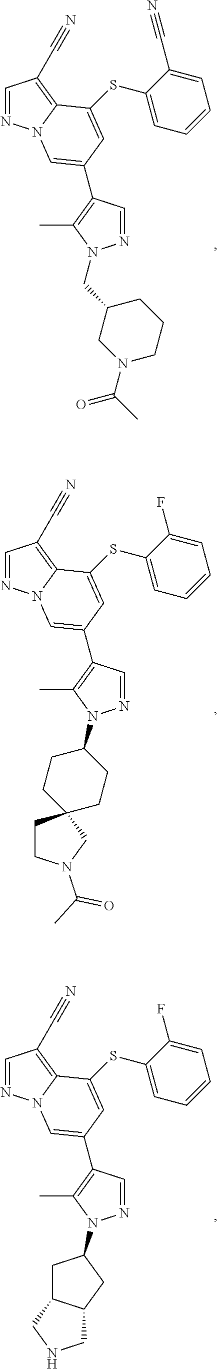 [SMILES: CC(=O)N1CCC[C@@H](Cn2ncc(-c3cc(Sc4ccccc4C#N)c4c(C#N)cnn4c3)c2C)C1 ; CC(=O)N1CC[C@]2(CC[C@@H](n3ncc(-c4cc(Sc5ccccc5F)c5c(C#N)cnn5c4)c3C)CC2)C1 ; Cc1c(-c2cc(Sc3ccccc3F)c3c(C#N)cnn3c2)cnn1[C@@H]1C[C@H]2CNC[C@H]2C1]

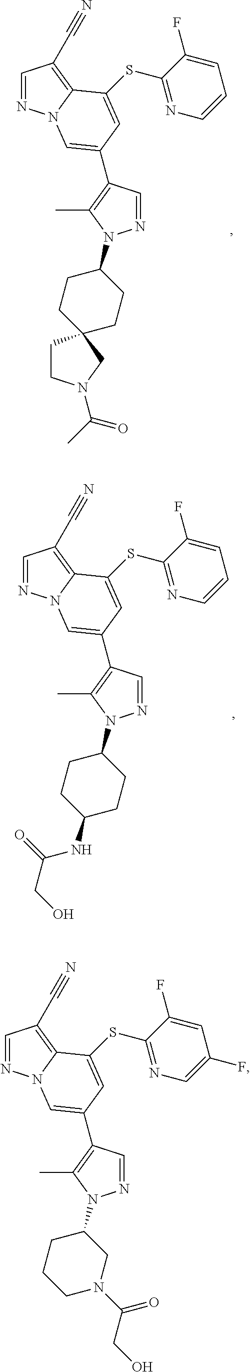 [SMILES: CC(=O)N1CC[C@]2(CC[C@H](n3ncc(-c4cc(Sc5ncccc5F)c5c(C#N)cnn5c4)c3C)CC2)C1 ; Cc1c(-c2cc(Sc3ncccc3F)c3c(C#N)cnn3c2)cnn1[C@H]1CC[C@@H](NC(=O)CO)CC1 ; Cc1c(-c2cc(Sc3ncc(F)cc3F)c3c(C#N)cnn3c2)cnn1[C@H]1CCCN(C(=O)CO)C1]

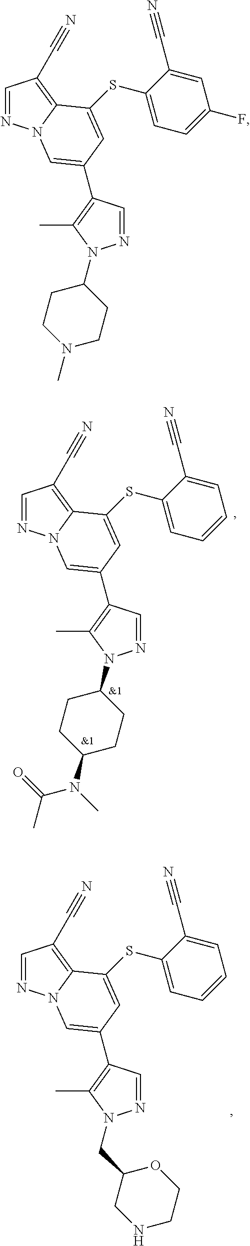 [SMILES: Cc1c(-c2cc(Sc3ccc(F)cc3C#N)c3c(C#N)cnn3c2)cnn1C1CCN(C)CC1 ; CC(=O)N(C)[C@H]1CC[C@@H](n2ncc(-c3cc(Sc4ccccc4C#N)c4c(C#N)cnn4c3)c2C)CC1 ; Cc1c(-c2cc(Sc3ccccc3C#N)c3c(C#N)cnn3c2)cnn1C[C@@H]1CNCCO1]

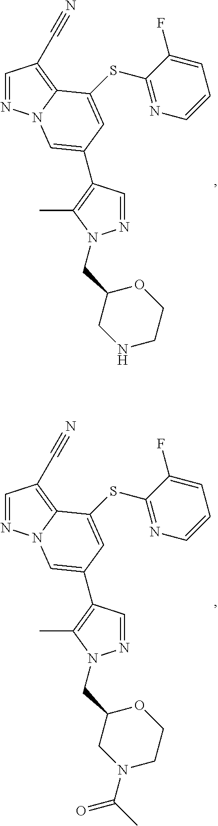 [SMILES: Cc1c(-c2cc(Sc3ncccc3F)c3c(C#N)cnn3c2)cnn1C[C@@H]1CNCCO1 ; CC(=O)N1CCO[C@H](Cn2ncc(-c3cc(Sc4ncccc4F)c4c(C#N)cnn4c3)c2C)C1]

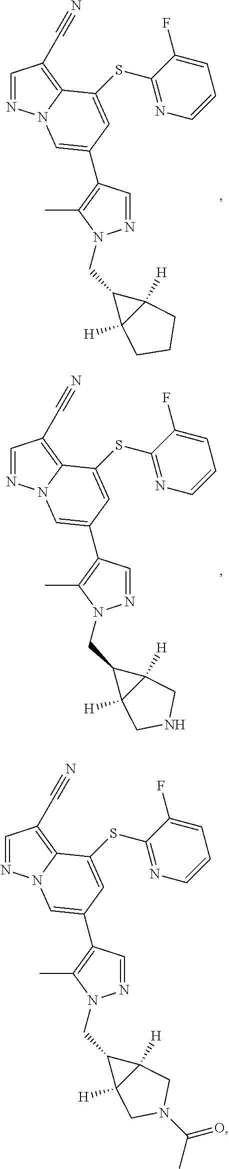 [SMILES: Cc1c(-c2cc(Sc3ncccc3F)c3c(C#N)cnn3c2)cnn1C[C@H]1[C@@H]2CCC[C@@H]21 ; Cc1c(-c2cc(Sc3ncccc3F)c3c(C#N)cnn3c2)cnn1C[C@@H]1[C@@H]2CNC[C@@H]21 ; CC(=O)N1C[C@@H]2[C@H](C1)[C@H]2Cn1ncc(-c2cc(Sc3ncccc3F)c3c(C#N)cnn3c2)c1C]

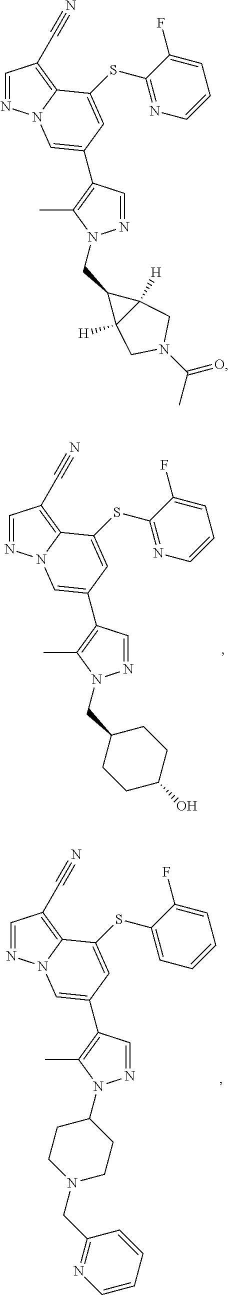 [SMILES: CC(=O)N1C[C@@H]2[C@H](C1)[C@@H]2Cn1ncc(-c2cc(Sc3ncccc3F)c3c(C#N)cnn3c2)c1C ; Cc1c(-c2cc(Sc3ncccc3F)c3c(C#N)cnn3c2)cnn1C[C@H]1CC[C@H](O)CC1 ; Cc1c(-c2cc(Sc3ccccc3F)c3c(C#N)cnn3c2)cnn1C1CCN(Cc2ccccn2)CC1]

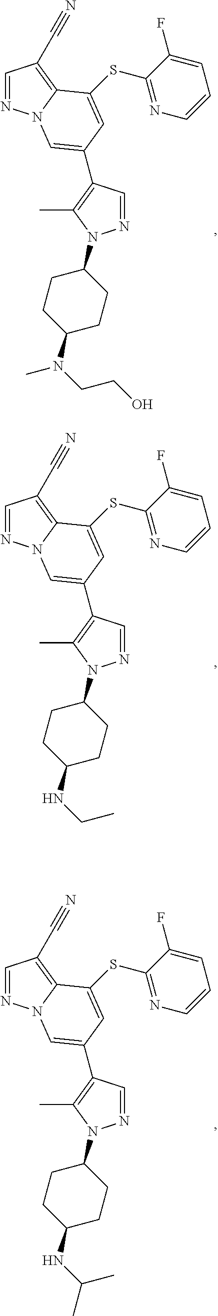 [SMILES: Cc1c(-c2cc(Sc3ncccc3F)c3c(C#N)cnn3c2)cnn1[C@H]1CC[C@@H](N(C)CCO)CC1 ; CCN[C@H]1CC[C@@H](n2ncc(-c3cc(Sc4ncccc4F)c4c(C#N)cnn4c3)c2C)CC1 ; Cc1c(-c2cc(Sc3ncccc3F)c3c(C#N)cnn3c2)cnn1[C@H]1CC[C@@H](NC(C)C)CC1]

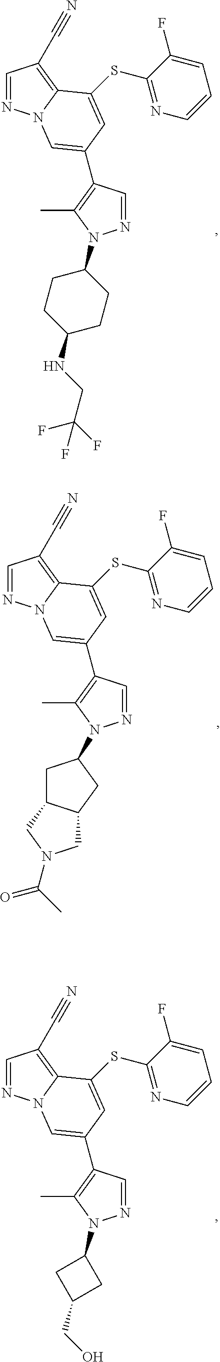

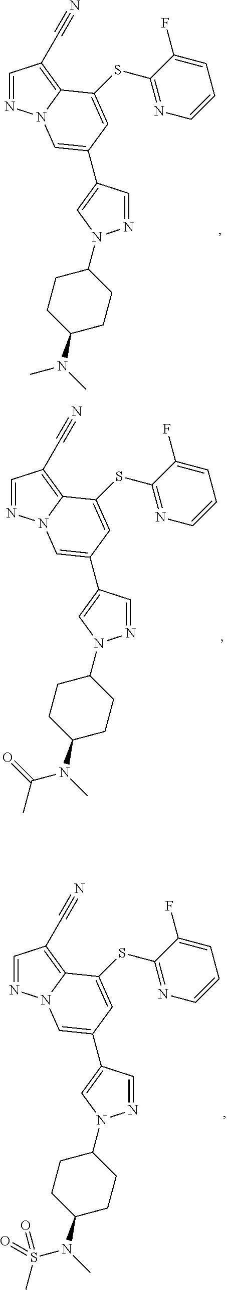

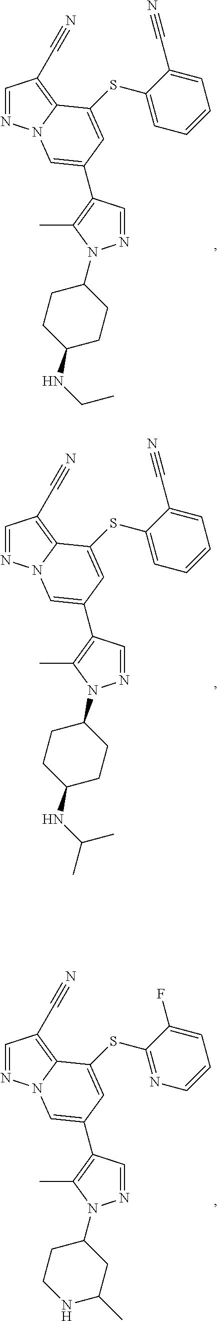

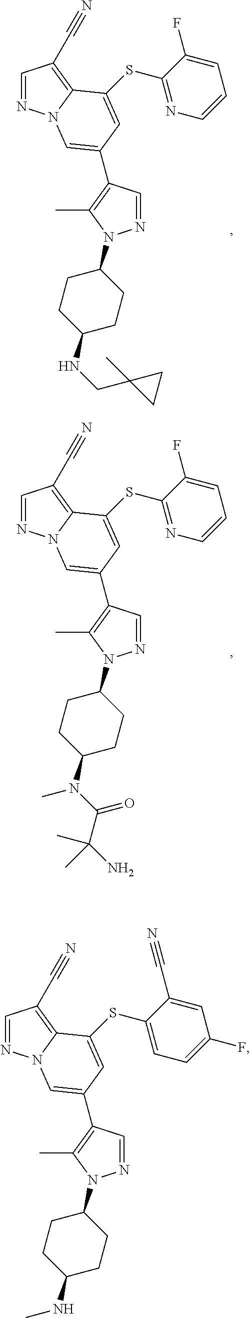

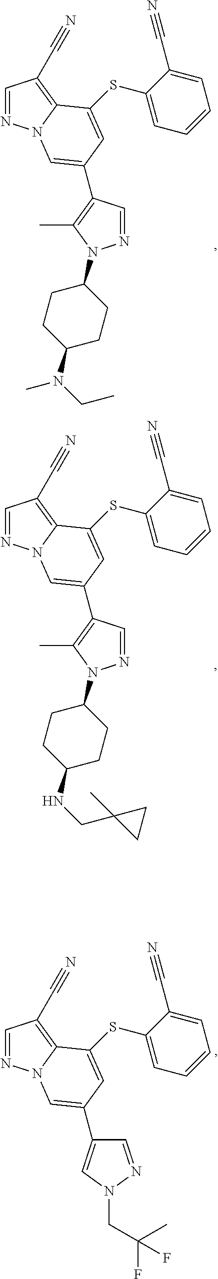

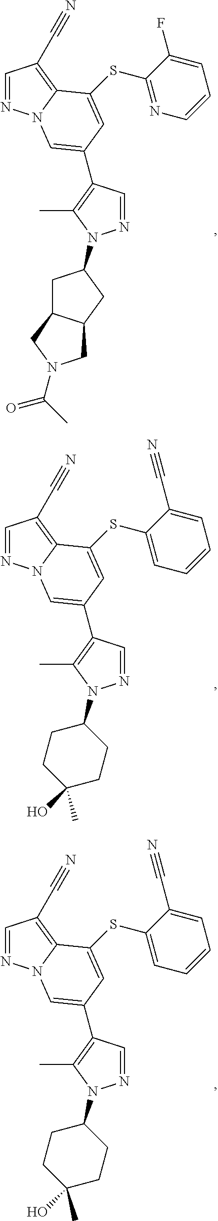

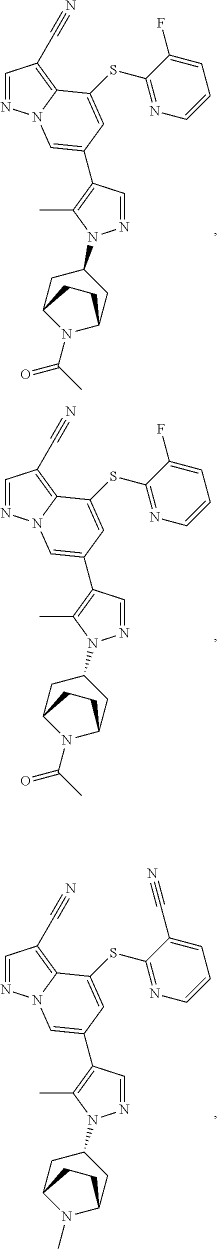

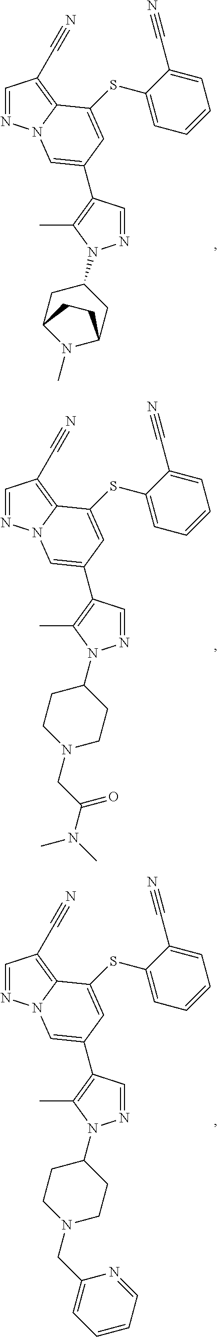

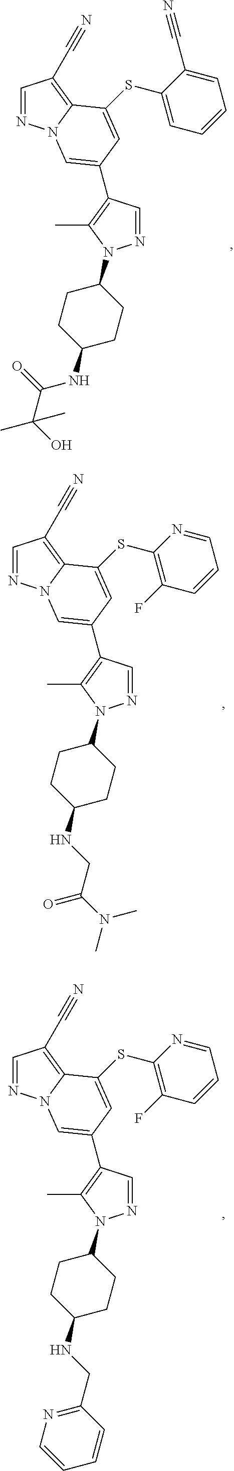

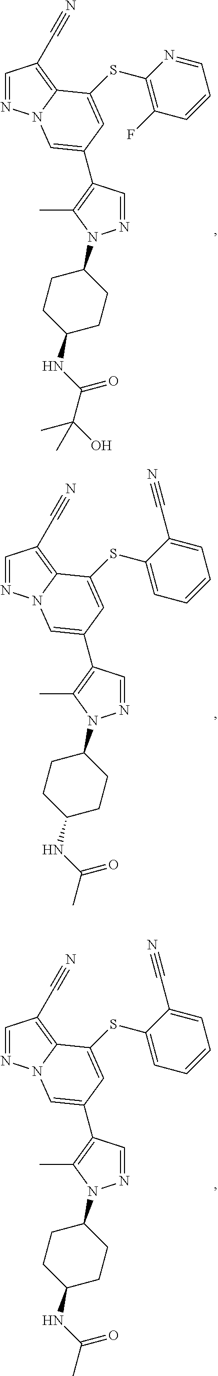

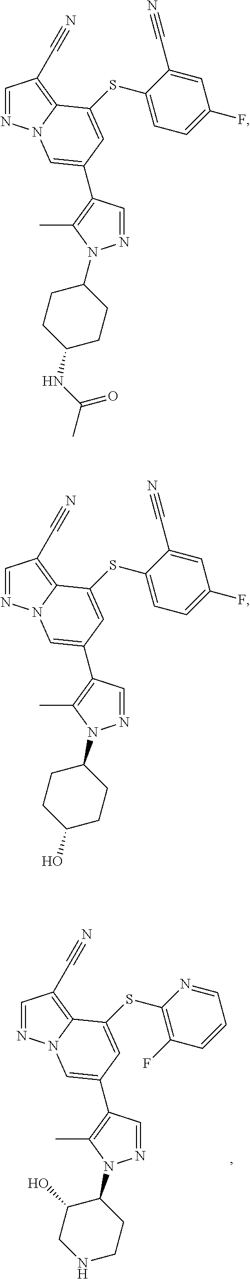

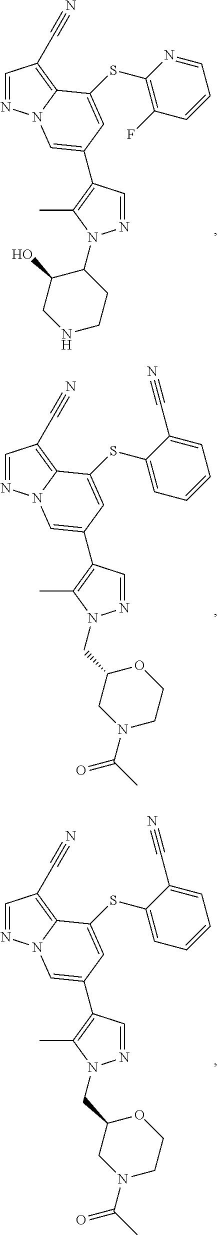

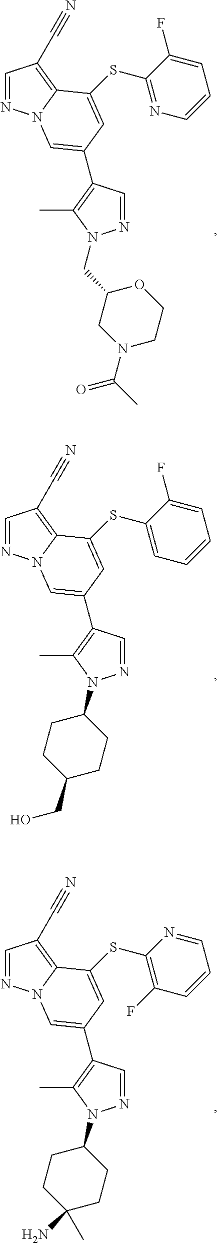 [SMILES: CC(=O)N1CCO[C@@H](Cn2ncc(-c3cc(Sc4ncccc4F)c4c(C#N)cnn4c3)c2C)C1 ; Cc1c(-c2cc(Sc3ccccc3F)c3c(C#N)cnn3c2)cnn1[C@H]1CC[C@@H](CO)CC1 ; Cc1c(-c2cc(Sc3ncccc3F)c3c(C#N)cnn3c2)cnn1[C@H]1CC[C@@](C)(N)CC1]

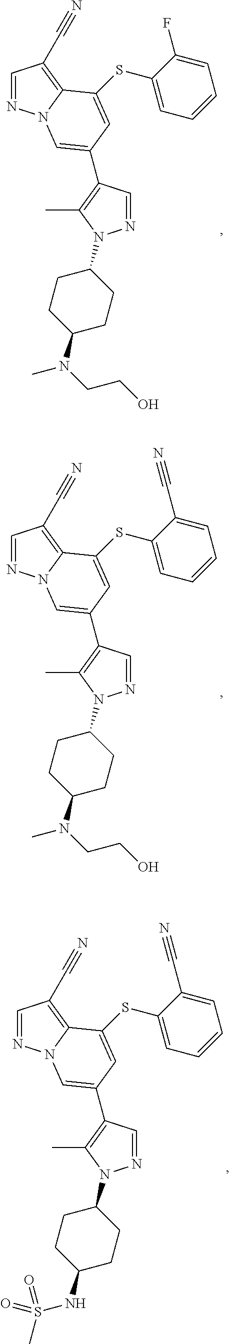 [SMILES: Cc1c(-c2cc(Sc3ccccc3F)c3c(C#N)cnn3c2)cnn1[C@H]1CC[C@H](N(C)CCO)CC1 ; Cc1c(-c2cc(Sc3ccccc3C#N)c3c(C#N)cnn3c2)cnn1[C@H]1CC[C@H](N(C)CCO)CC1 ; Cc1c(-c2cc(Sc3ccccc3C#N)c3c(C#N)cnn3c2)cnn1[C@H]1CC[C@@H](NS(C)(=O)=O)CC1]

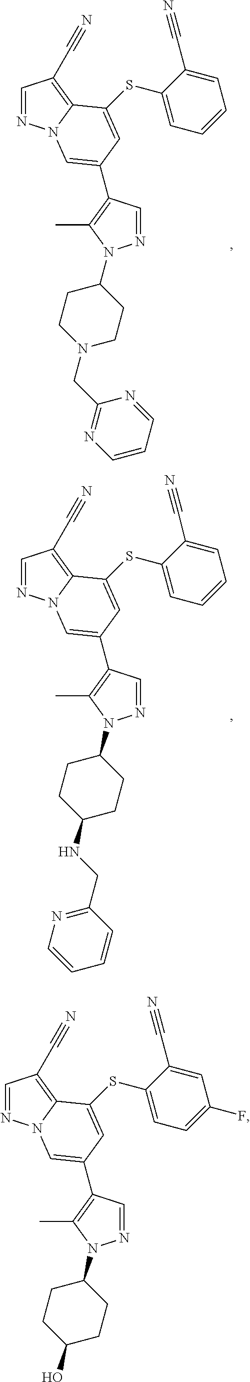

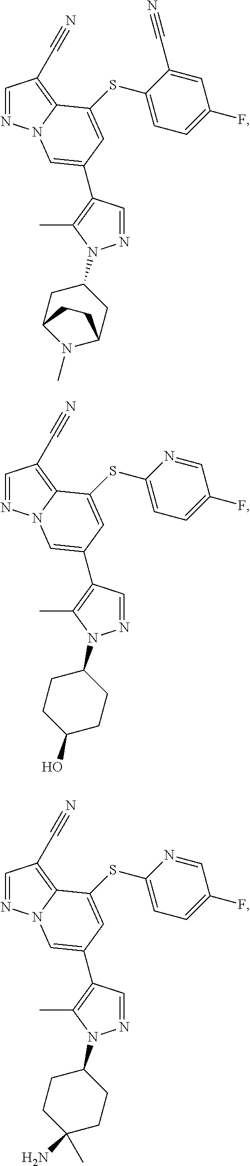

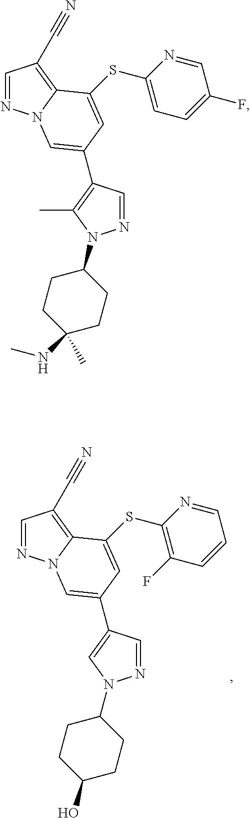

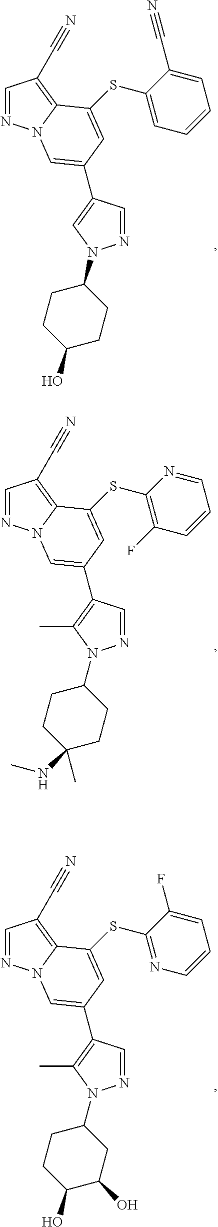

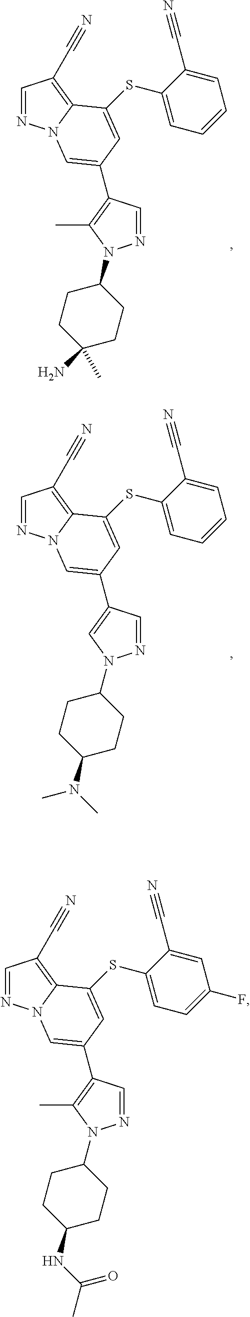

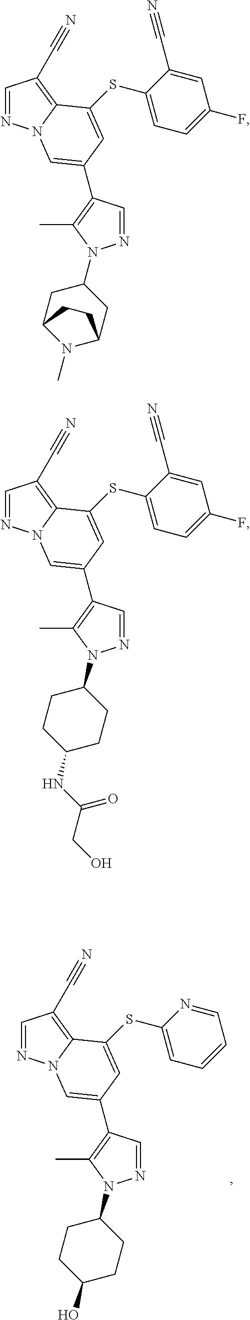

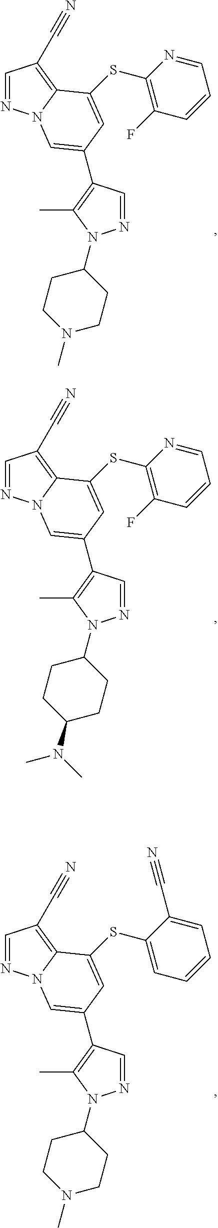

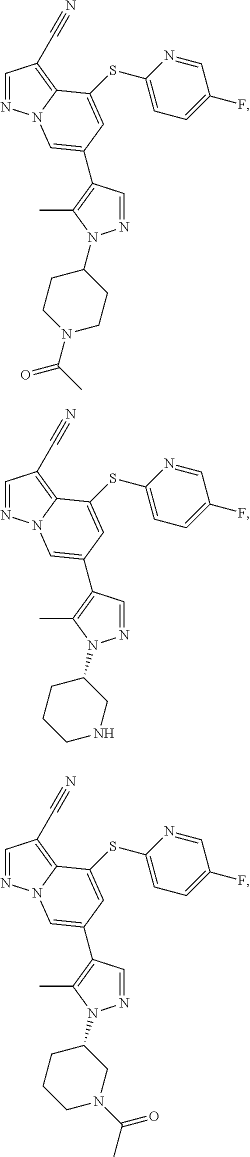

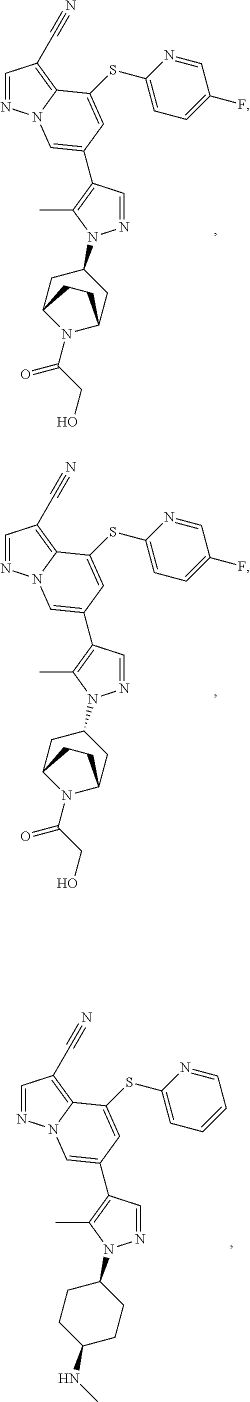

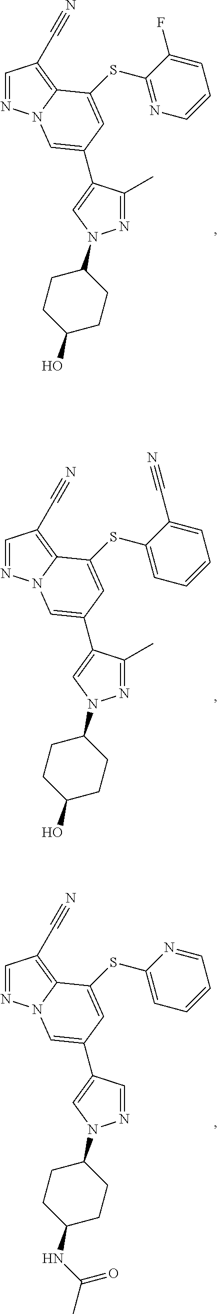

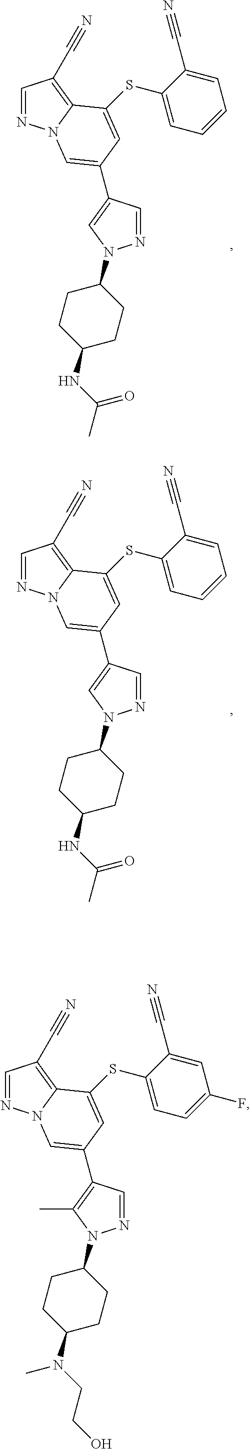 [SMILES: CC(=O)N[C@H]1CC[C@@H](n2cc(-c3cc(Sc4ccccc4C#N)c4c(C#N)cnn4c3)cn2)CC1 ; CC(=O)N[C@H]1CC[C@@H](n2cc(-c3cc(Sc4ccccc4C#N)c4c(C#N)cnn4c3)cn2)CC1 ; Cc1c(-c2cc(Sc3ccc(F)cc3C#N)c3c(C#N)cnn3c2)cnn1[C@H]1CC[C@@H](N(C)CCO)CC1]

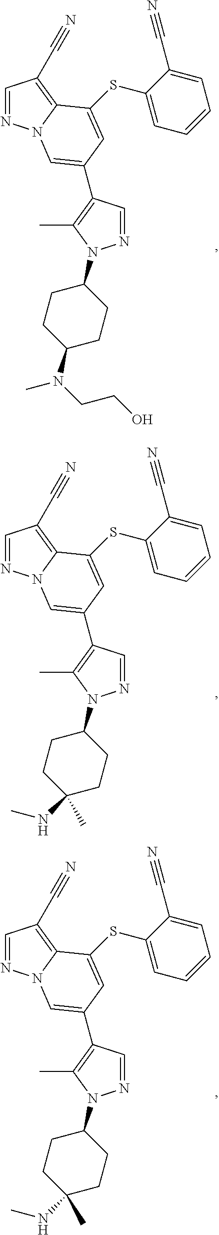

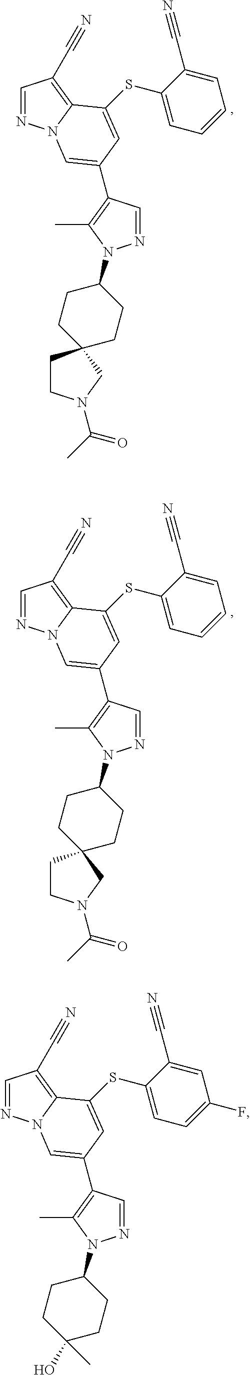 [SMILES: CC(=O)N1CC[C@]2(CC[C@@H](n3ncc(-c4cc(Sc5ccccc5C#N)c5c(C#N)cnn5c4)c3C)CC2)C1 ; CC(=O)N1CC[C@]2(CC[C@H](n3ncc(-c4cc(Sc5ccccc5C#N)c5c(C#N)cnn5c4)c3C)CC2)C1 ; Cc1c(-c2cc(Sc3ccc(F)cc3C#N)c3c(C#N)cnn3c2)cnn1[C@H]1CC[C@](C)(O)CC1]

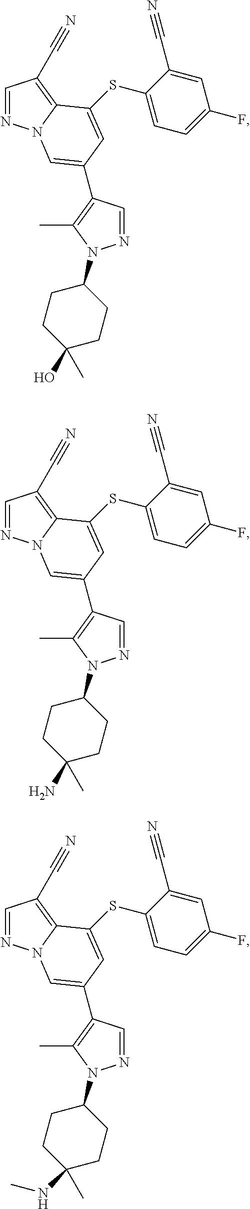 [SMILES: Cc1c(-c2cc(Sc3ccc(F)cc3C#N)c3c(C#N)cnn3c2)cnn1[C@H]1CC[C@@](C)(O)CC1 ; Cc1c(-c2cc(Sc3ccc(F)cc3C#N)c3c(C#N)cnn3c2)cnn1[C@H]1CC[C@@](C)(N)CC1 ; CN[C@]1(C)CC[C@H](n2ncc(-c3cc(Sc4ccc(F)cc4C#N)c4c(C#N)cnn4c3)c2C)CC1]

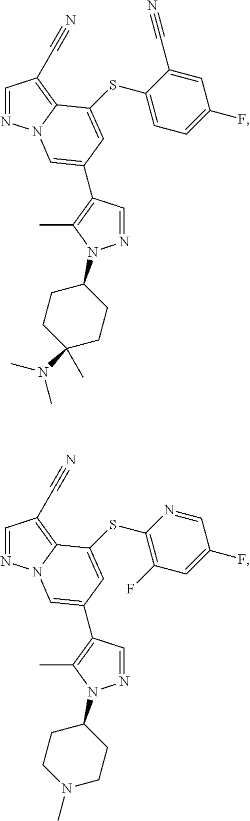

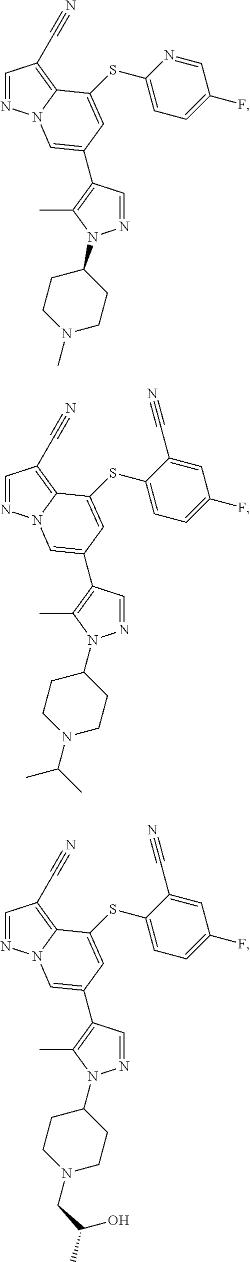

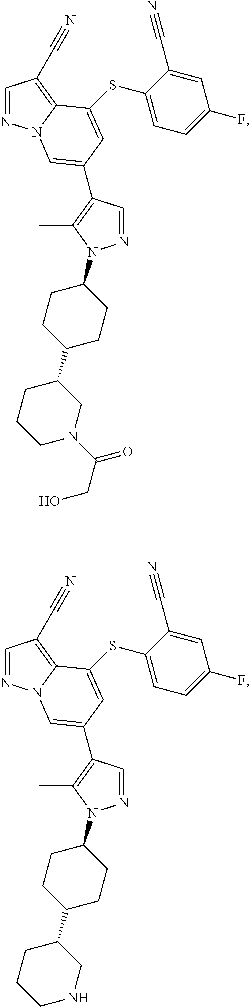

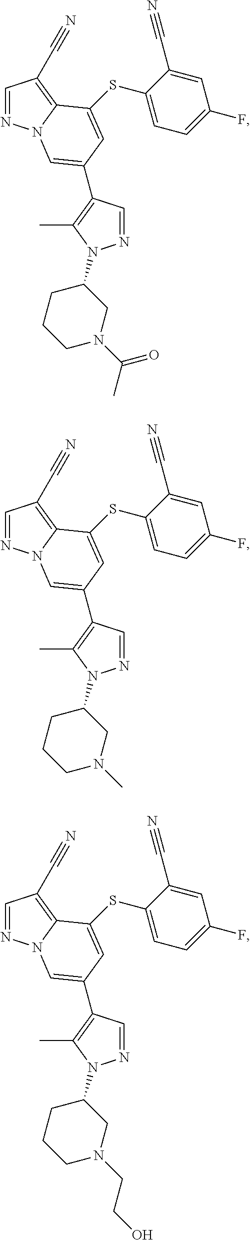

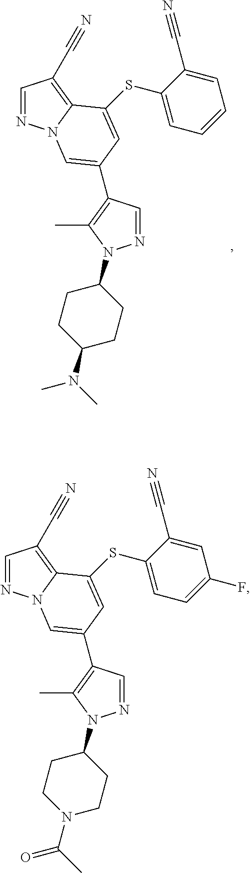

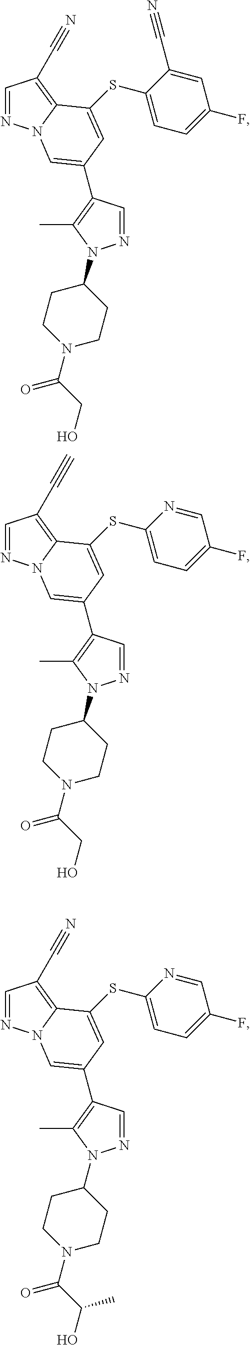

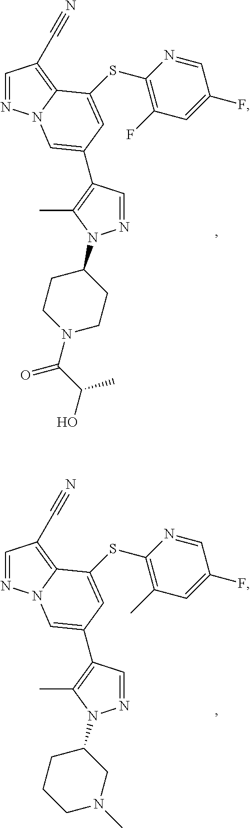

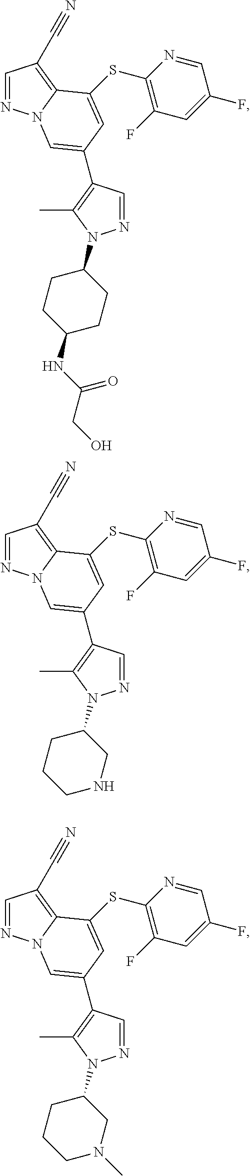

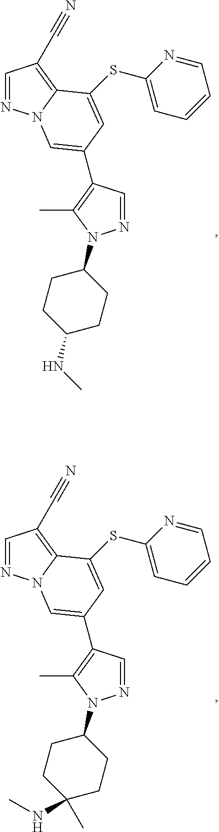 [SMILES: CN[C@H]1CC[C@H](n2ncc(-c3cc(Sc4ccccn4)c4c(C#N)cnn4c3)c2C)CC1 ; CN[C@]1(C)CC[C@H](n2ncc(-c3cc(Sc4ccccn4)c4c(C#N)cnn4c3)c2C)CC1]

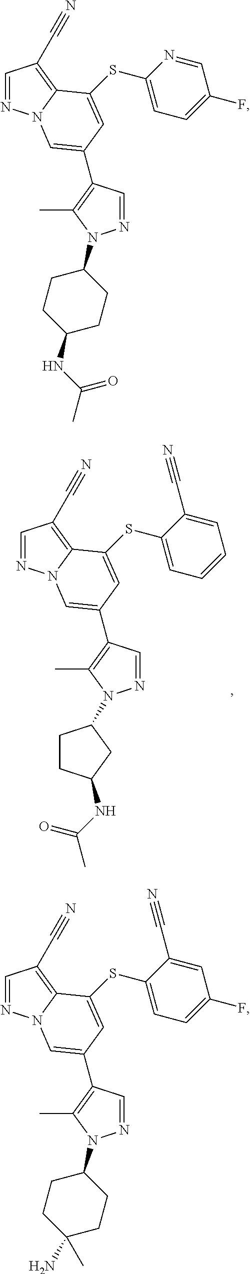

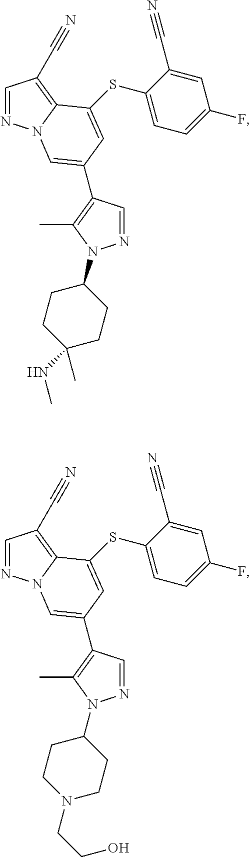 [SMILES: CN[C@]1(C)CC[C@@H](n2ncc(-c3cc(Sc4ccc(F)cc4C#N)c4c(C#N)cnn4c3)c2C)CC1 ; Cc1c(-c2cc(Sc3ccc(F)cc3C#N)c3c(C#N)cnn3c2)cnn1C1CCN(CCO)CC1]

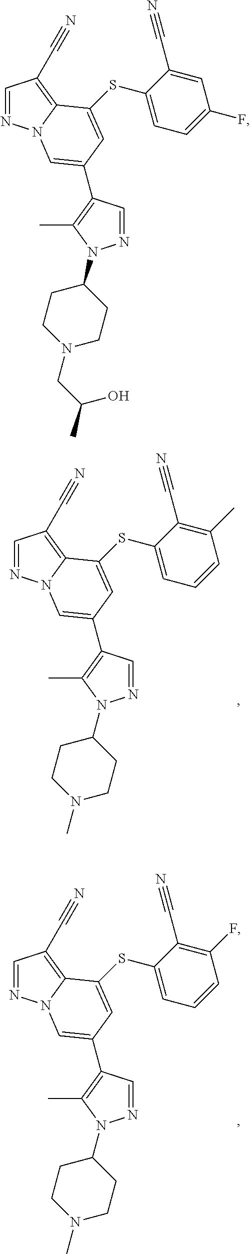

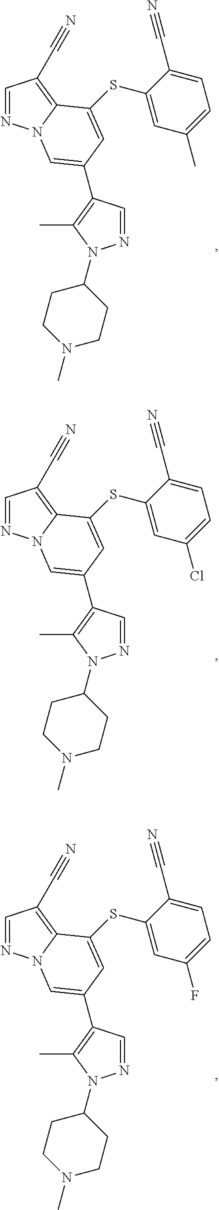 [SMILES: Cc1ccc(C#N)c(Sc2cc(-c3cnn(C4CCN(C)CC4)c3C)cn3ncc(C#N)c23)c1 ; Cc1c(-c2cc(Sc3cc(Cl)ccc3C#N)c3c(C#N)cnn3c2)cnn1C1CCN(C)CC1 ; Cc1c(-c2cc(Sc3cc(F)ccc3C#N)c3c(C#N)cnn3c2)cnn1C1CCN(C)CC1]

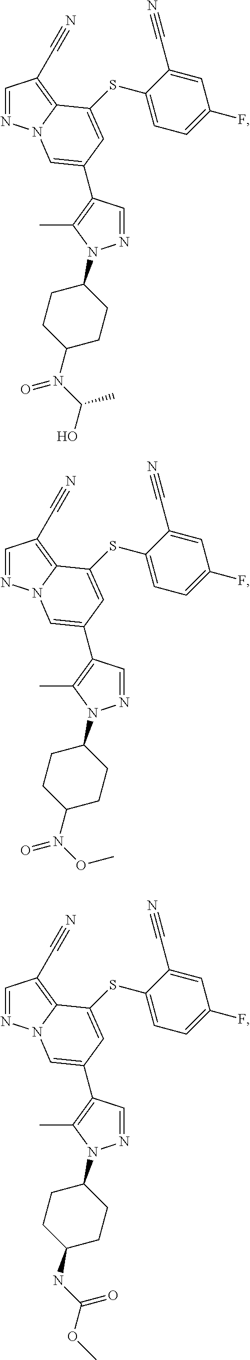

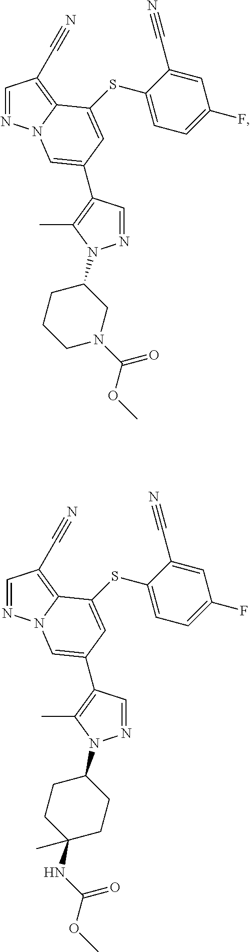 [SMILES: COC(=O)N1CCC[C@H](n2ncc(-c3cc(Sc4ccc(F)cc4C#N)c4c(C#N)cnn4c3)c2C)C1 ; COC(=O)N[C@]1(C)CC[C@H](n2ncc(-c3cc(Sc4ccc(F)cc4C#N)c4c(C#N)cnn4c3)c2C)CC1]

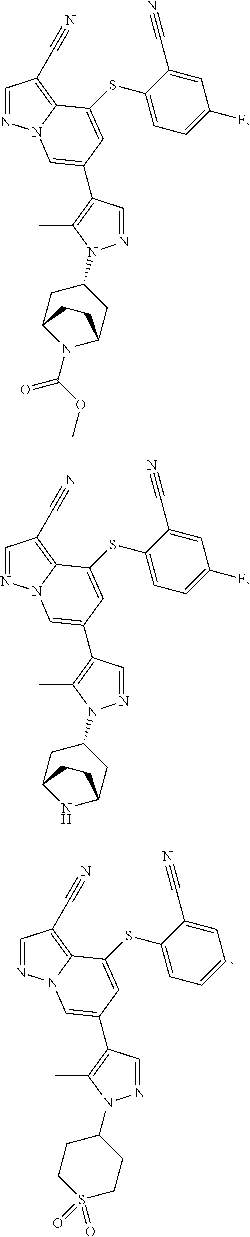

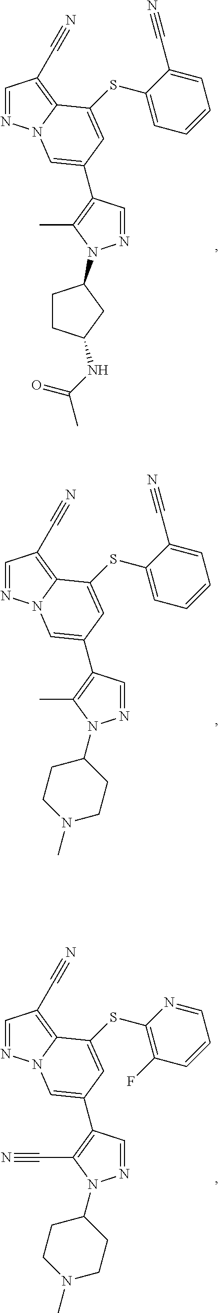 [SMILES: CC(=O)N[C@@H]1CC[C@@H](n2ncc(-c3cc(Sc4ccccc4C#N)c4c(C#N)cnn4c3)c2C)C1 ; Cc1c(-c2cc(Sc3ccccc3C#N)c3c(C#N)cnn3c2)cnn1C1CCN(C)CC1 ; CN1CCC(n2ncc(-c3cc(Sc4ncccc4F)c4c(C#N)cnn4c3)c2C#N)CC1]

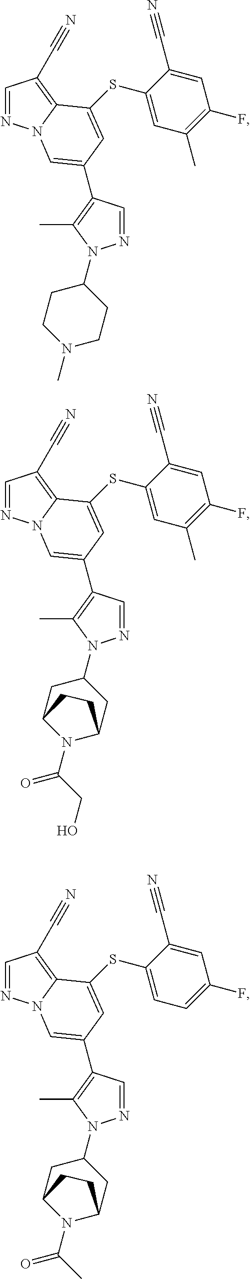

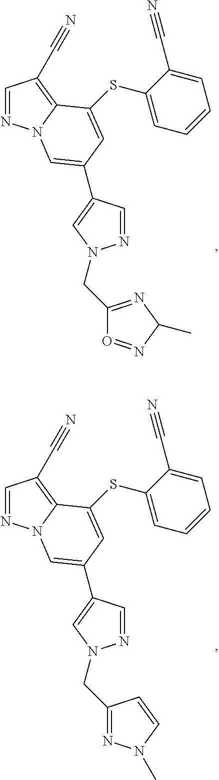

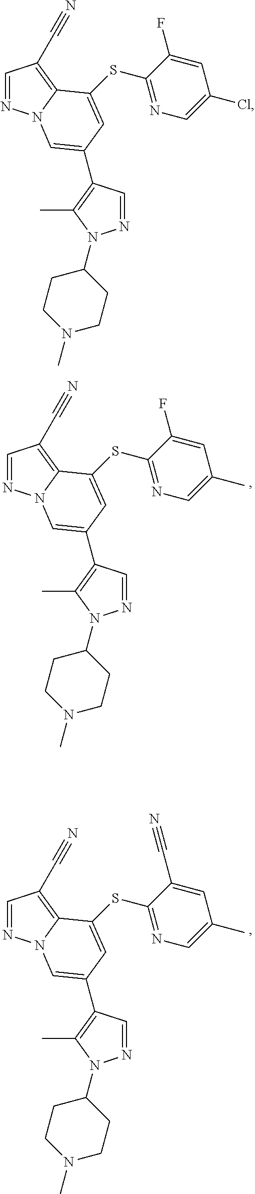

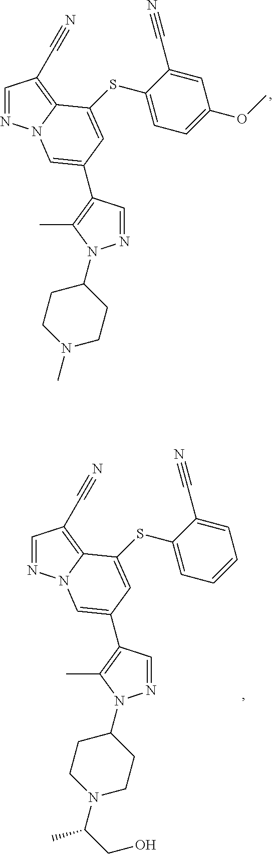 [SMILES: COc1ccc(Sc2cc(-c3cnn(C4CCN(C)CC4)c3C)cn3ncc(C#N)c23)c(C#N)c1 ; Cc1c(-c2cc(Sc3ccccc3C#N)c3c(C#N)cnn3c2)cnn1C1CCN([C@@H](C)CO)CC1]

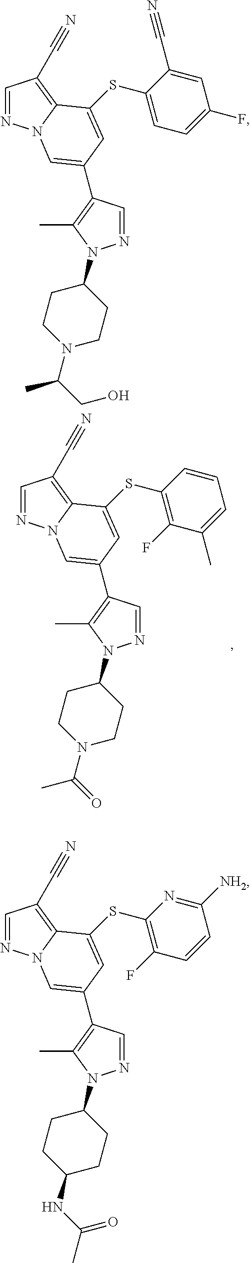

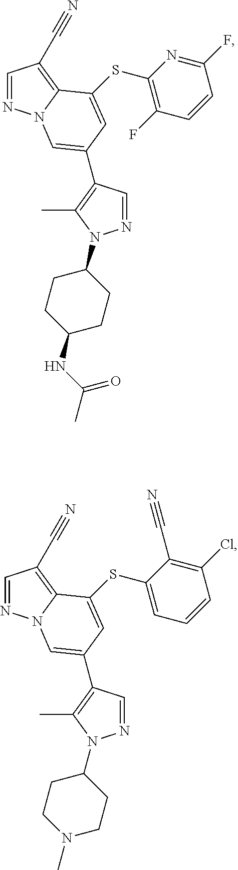 [SMILES: CC(=O)N[C@H]1CC[C@@H](n2ncc(-c3cc(Sc4nc(F)ccc4F)c4c(C#N)cnn4c3)c2C)CC1 ; Cc1c(-c2cc(Sc3cccc(Cl)c3C#N)c3c(C#N)cnn3c2)cnn1C1CCN(C)CC1]

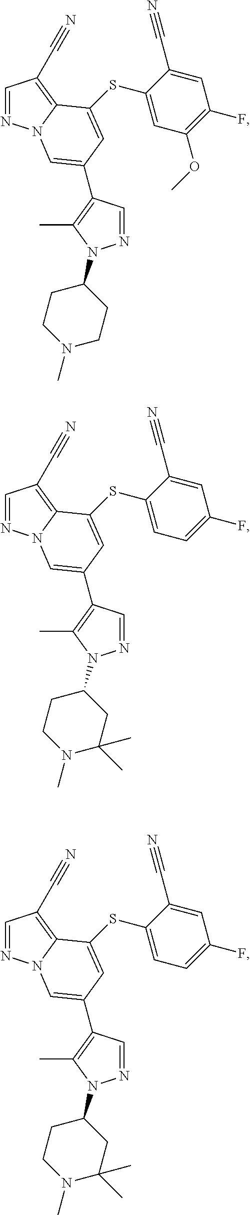

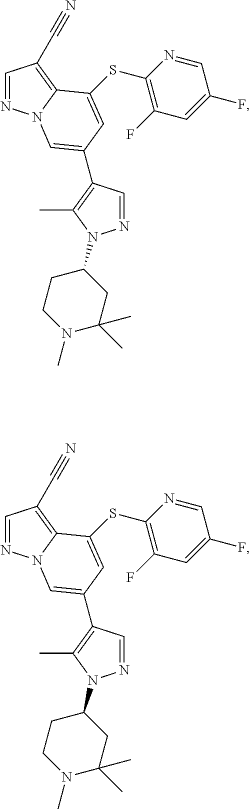 [SMILES: Cc1c(-c2cc(Sc3ncc(F)cc3F)c3c(C#N)cnn3c2)cnn1[C@H]1CCN(C)C(C)(C)C1 ; Cc1c(-c2cc(Sc3ncc(F)cc3F)c3c(C#N)cnn3c2)cnn1[C@@H]1CCN(C)C(C)(C)C1]

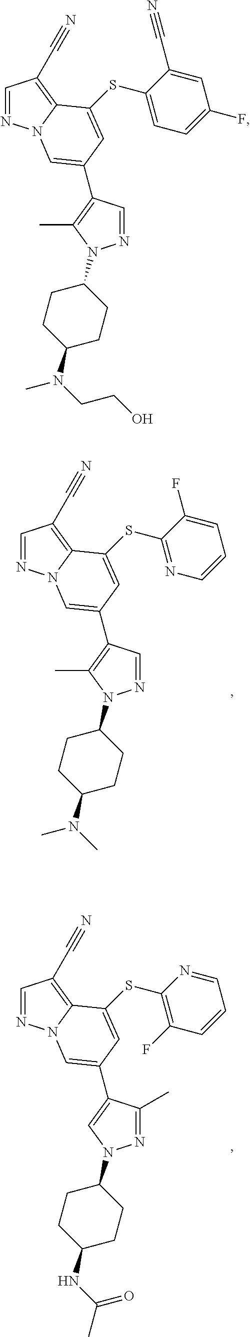 [SMILES: Cc1c(-c2cc(Sc3ccc(F)cc3C#N)c3c(C#N)cnn3c2)cnn1[C@H]1CC[C@H](N(C)CCO)CC1 ; Cc1c(-c2cc(Sc3ncccc3F)c3c(C#N)cnn3c2)cnn1[C@H]1CC[C@@H](N(C)C)CC1 ; CC(=O)N[C@H]1CC[C@@H](n2cc(-c3cc(Sc4ncccc4F)c4c(C#N)cnn4c3)c(C)n2)CC1]

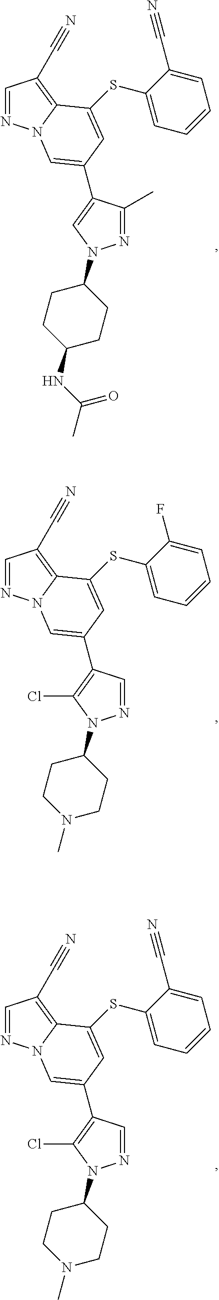

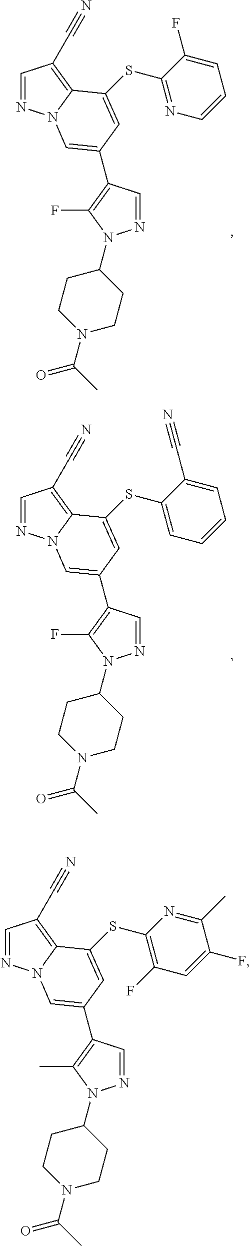

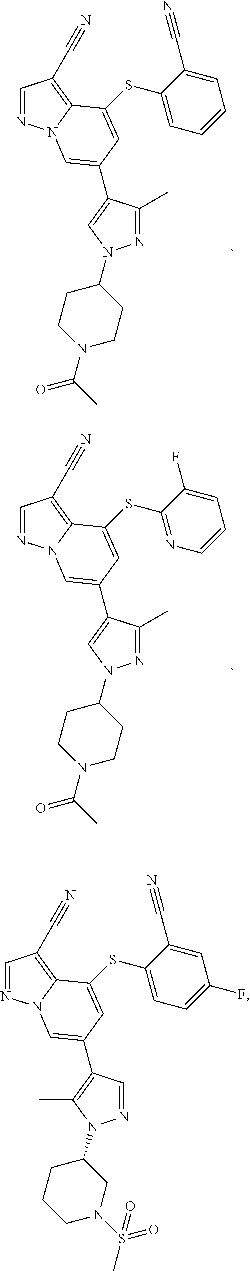

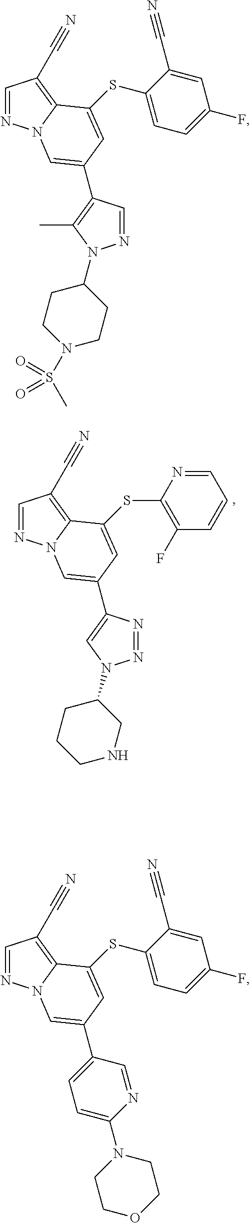

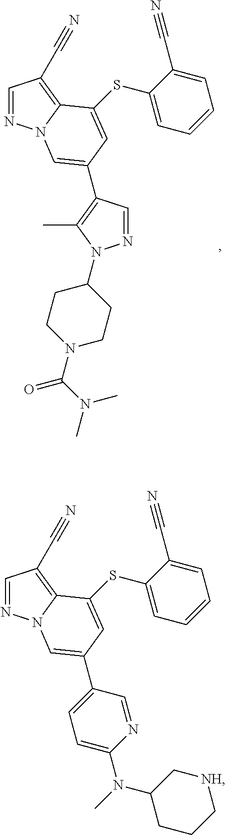 [SMILES: Cc1c(-c2cc(Sc3ccccc3C#N)c3c(C#N)cnn3c2)cnn1C1CCN(C(=O)N(C)C)CC1 ; CN(c1ccc(-c2cc(Sc3ccccc3C#N)c3c(C#N)cnn3c2)cn1)C1CCCNC1]

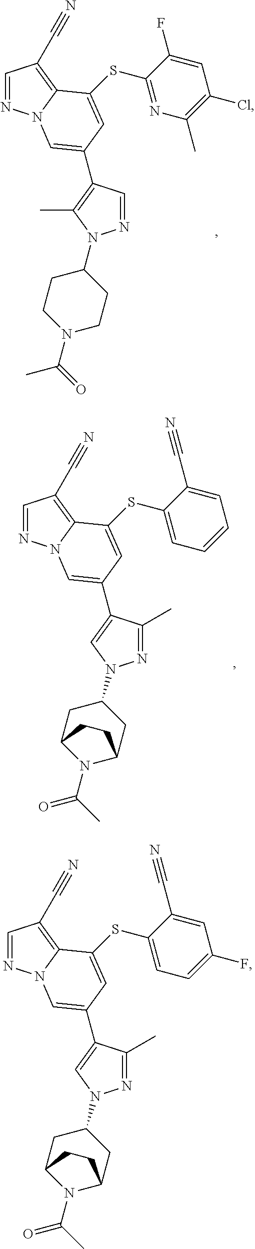 [SMILES: CC(=O)N1CCC(n2ncc(-c3cc(Sc4nc(C)c(Cl)cc4F)c4c(C#N)cnn4c3)c2C)CC1 ; CC(=O)N1[C@@H]2CC[C@H]1C[C@H](n1cc(-c3cc(Sc4ccccc4C#N)c4c(C#N)cnn4c3)c(C)n1)C2 ; CC(=O)N1[C@@H]2CC[C@H]1C[C@H](n1cc(-c3cc(Sc4ccc(F)cc4C#N)c4c(C#N)cnn4c3)c(C)n1)C2]

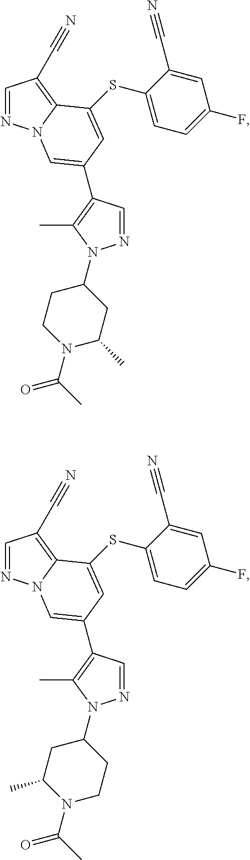 [SMILES: CC(=O)N1CCC(n2ncc(-c3cc(Sc4ccc(F)cc4C#N)c4c(C#N)cnn4c3)c2C)C[C@@H]1C ; CC(=O)N1CCC(n2ncc(-c3cc(Sc4ccc(F)cc4C#N)c4c(C#N)cnn4c3)c2C)C[C@H]1C]

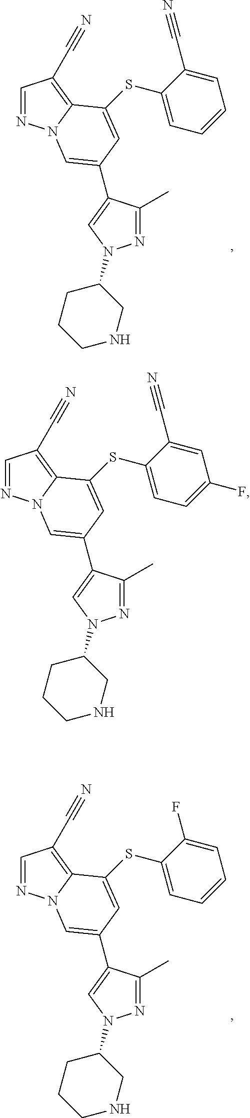

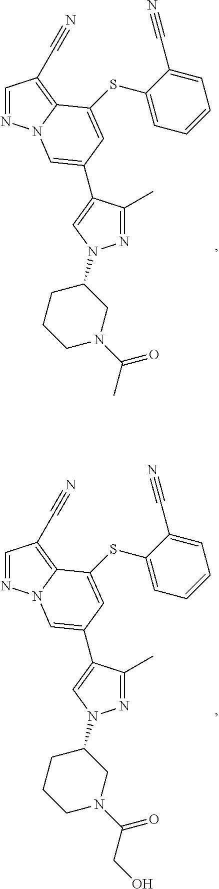 [SMILES: CC(=O)N1CCC[C@H](n2cc(-c3cc(Sc4ccccc4C#N)c4c(C#N)cnn4c3)c(C)n2)C1 ; Cc1nn([C@H]2CCCN(C(=O)CO)C2)cc1-c1cc(Sc2ccccc2C#N)c2c(C#N)cnn2c1]

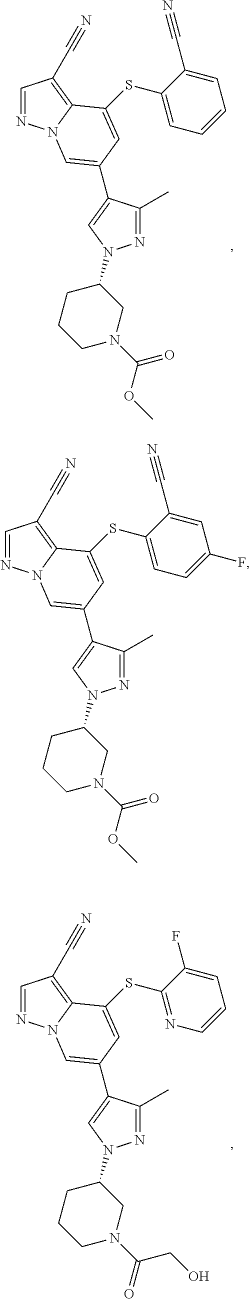

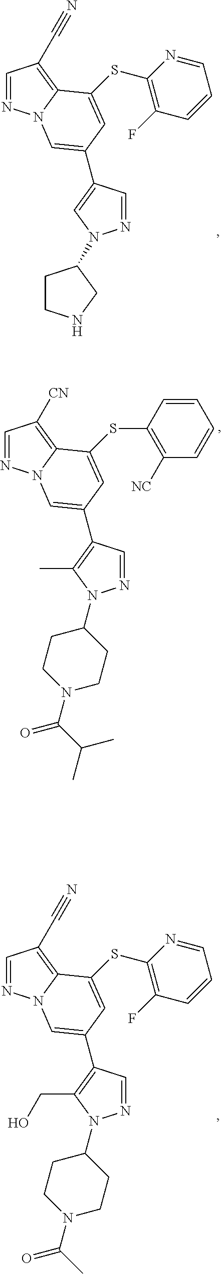 [SMILES: N#Cc1cnn2cc(-c3cnn([C@H]4CCNC4)c3)cc(Sc3ncccc3F)c12 ; Cc1c(-c2cc(Sc3ccccc3C#N)c3c(C#N)cnn3c2)cnn1C1CCN(C(=O)C(C)C)CC1 ; CC(=O)N1CCC(n2ncc(-c3cc(Sc4ncccc4F)c4c(C#N)cnn4c3)c2CO)CC1]

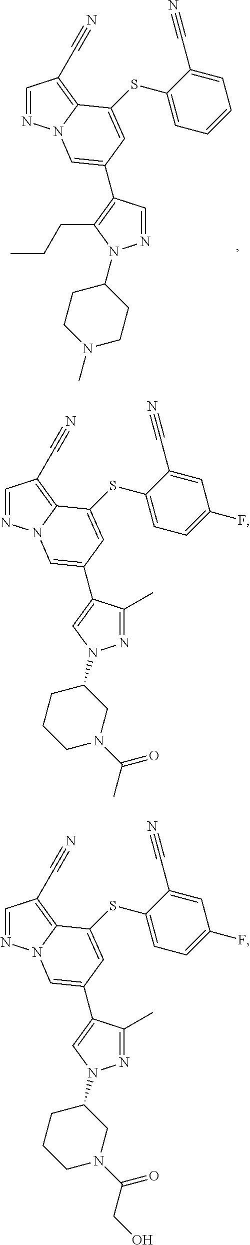

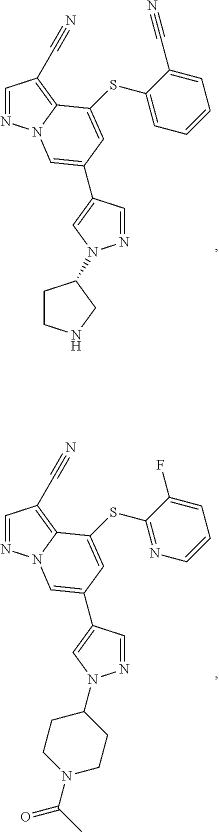 [SMILES: N#Cc1ccccc1Sc1cc(-c2cnn([C@H]3CCNC3)c2)cn2ncc(C#N)c12 ; CC(=O)N1CCC(n2cc(-c3cc(Sc4ncccc4F)c4c(C#N)cnn4c3)cn2)CC1]

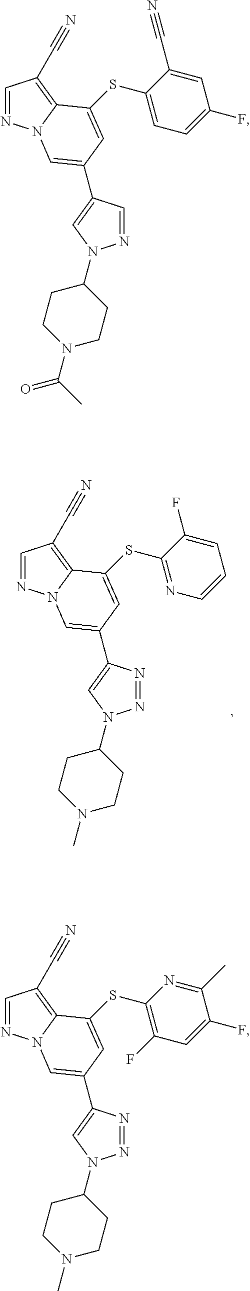 [SMILES: CC(=O)N1CCC(n2cc(-c3cc(Sc4ccc(F)cc4C#N)c4c(C#N)cnn4c3)cn2)CC1 ; CN1CCC(n2cc(-c3cc(Sc4ncccc4F)c4c(C#N)cnn4c3)nn2)CC1 ; Cc1nc(Sc2cc(-c3cn(C4CCN(C)CC4)nn3)cn3ncc(C#N)c23)c(F)cc1F]

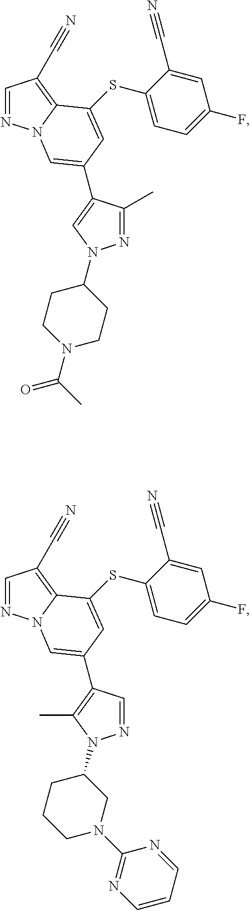 [SMILES: CC(=O)N1CCC(n2cc(-c3cc(Sc4ccc(F)cc4C#N)c4c(C#N)cnn4c3)c(C)n2)CC1 ; Cc1c(-c2cc(Sc3ccc(F)cc3C#N)c3c(C#N)cnn3c2)cnn1[C@H]1CCCN(c2ncccn2)C1]

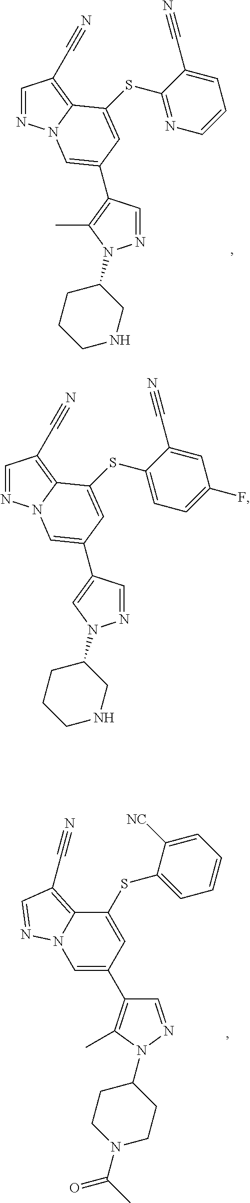 [SMILES: Cc1c(-c2cc(Sc3ncccc3C#N)c3c(C#N)cnn3c2)cnn1[C@H]1CCCNC1 ; N#Cc1cc(F)ccc1Sc1cc(-c2cnn([C@H]3CCCNC3)c2)cn2ncc(C#N)c12 ; CC(=O)N1CCC(n2ncc(-c3cc(Sc4ccccc4C#N)c4c(C#N)cnn4c3)c2C)CC1]

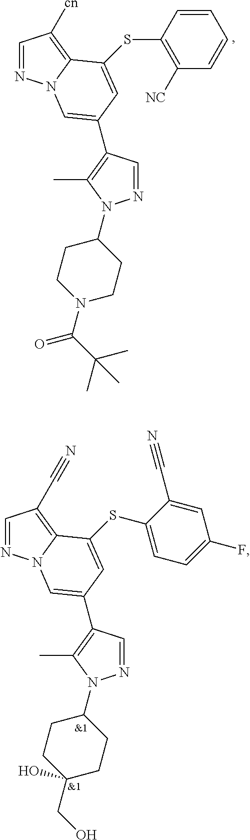

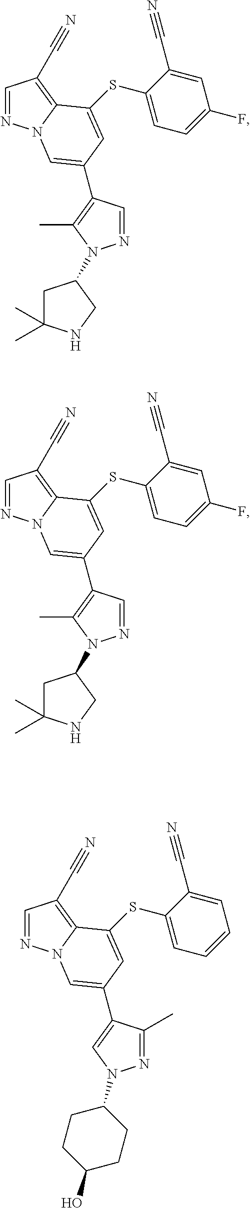 [SMILES: Cc1c(-c2cc(Sc3ccc(F)cc3C#N)c3c(C#N)cnn3c2)cnn1[C@@H]1CNC(C)(C)C1 ; Cc1c(-c2cc(Sc3ccc(F)cc3C#N)c3c(C#N)cnn3c2)cnn1[C@H]1CNC(C)(C)C1 ; Cc1nn([C@H]2CC[C@H](O)CC2)cc1-c1cc(Sc2ccccc2C#N)c2c(C#N)cnn2c1]

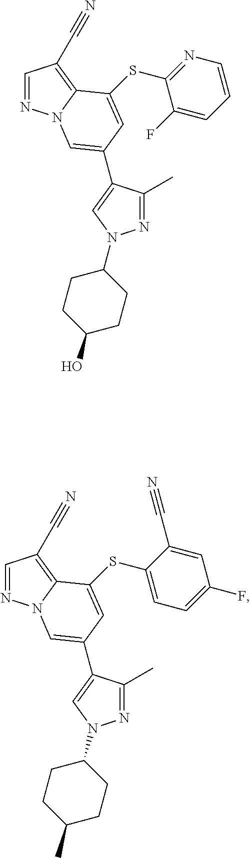

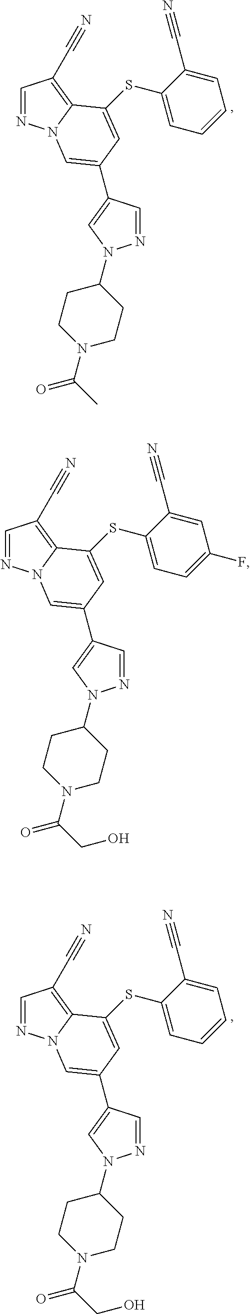 [SMILES: CC(=O)N1CCC(n2cc(-c3cc(Sc4ccccc4C#N)c4c(C#N)cnn4c3)cn2)CC1 ; N#Cc1cc(F)ccc1Sc1cc(-c2cnn(C3CCN(C(=O)CO)CC3)c2)cn2ncc(C#N)c12 ; N#Cc1ccccc1Sc1cc(-c2cnn(C3CCN(C(=O)CO)CC3)c2)cn2ncc(C#N)c12]

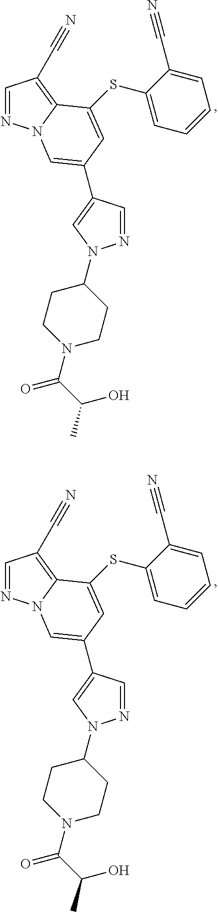 [SMILES: C[C@@H](O)C(=O)N1CCC(n2cc(-c3cc(Sc4ccccc4C#N)c4c(C#N)cnn4c3)cn2)CC1 ; C[C@H](O)C(=O)N1CCC(n2cc(-c3cc(Sc4ccccc4C#N)c4c(C#N)cnn4c3)cn2)CC1]

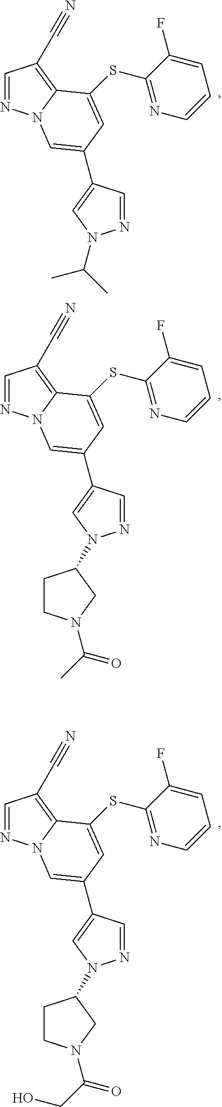

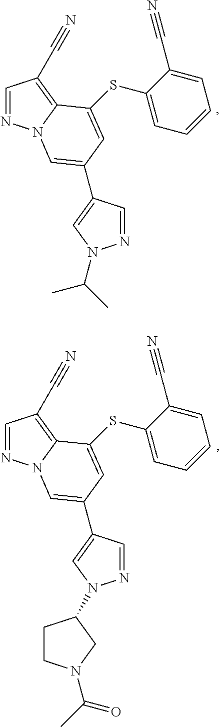 [SMILES: CC(C)n1cc(-c2cc(Sc3ccccc3C#N)c3c(C#N)cnn3c2)cn1 ; CC(=O)N1CC[C@H](n2cc(-c3cc(Sc4ccccc4C#N)c4c(C#N)cnn4c3)cn2)C1]

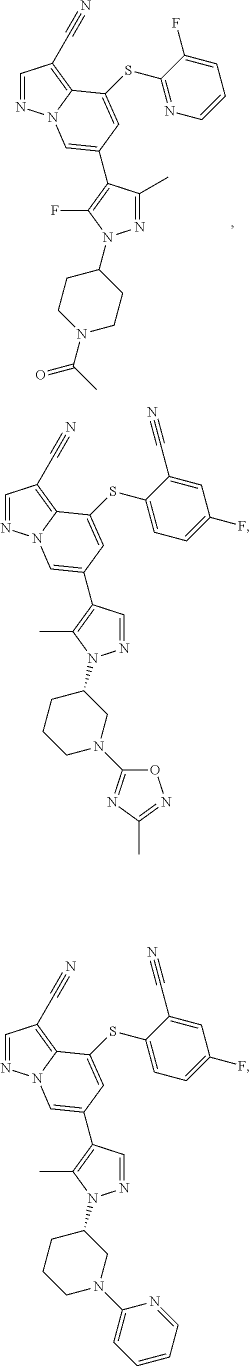

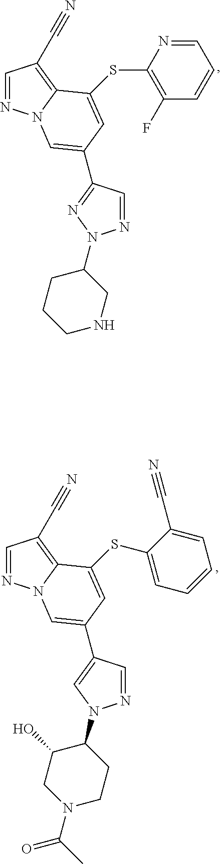 [SMILES: N#Cc1cnn2cc(-c3cnn(C4CCCNC4)n3)cc(Sc3ncccc3F)c12 ; CC(=O)N1CC[C@H](n2cc(-c3cc(Sc4ccccc4C#N)c4c(C#N)cnn4c3)cn2)[C@@H](O)C1]

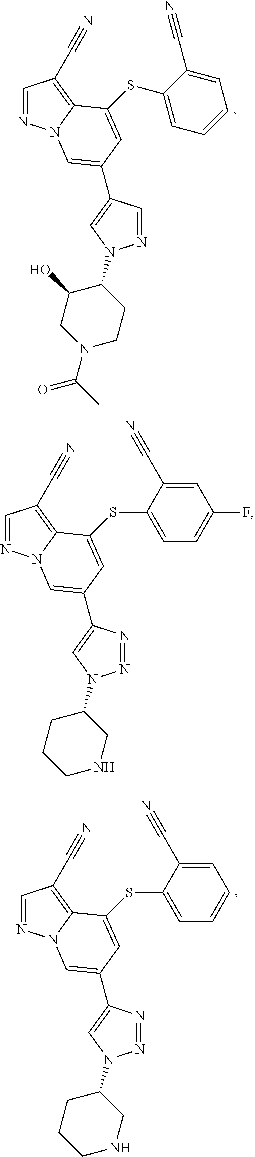

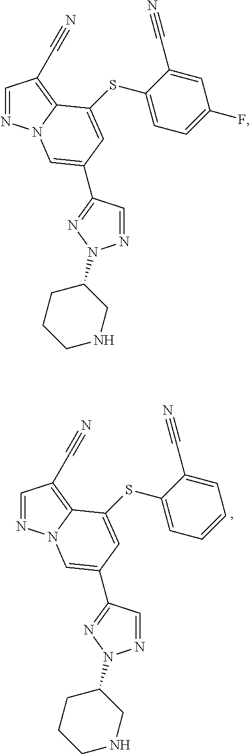 [SMILES: N#Cc1cc(F)ccc1Sc1cc(-c2cnn([C@H]3CCCNC3)n2)cn2ncc(C#N)c12 ; N#Cc1ccccc1Sc1cc(-c2cnn([C@H]3CCCNC3)n2)cn2ncc(C#N)c12]

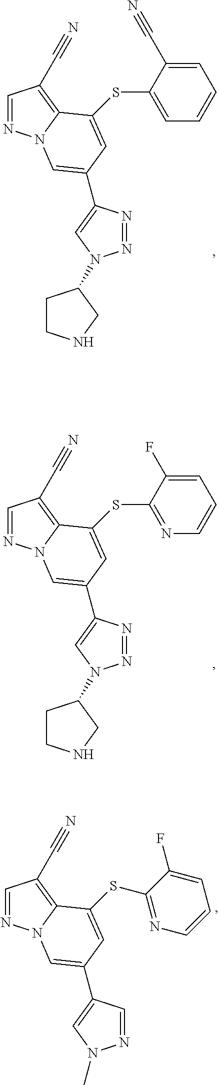 [SMILES: N#Cc1ccccc1Sc1cc(-c2cn([C@H]3CCNC3)nn2)cn2ncc(C#N)c12 ; N#Cc1cnn2cc(-c3cn([C@H]4CCNC4)nn3)cc(Sc3ncccc3F)c12 ; Cn1cc(-c2cc(Sc3ncccc3F)c3c(C#N)cnn3c2)cn1]

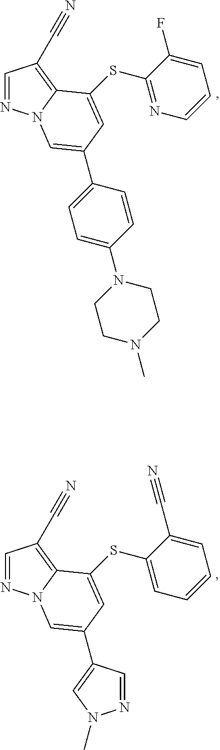 [SMILES: CN1CCN(c2ccc(-c3cc(Sc4ncccc4F)c4c(C#N)cnn4c3)cc2)CC1 ; Cn1cc(-c2cc(Sc3ccccc3C#N)c3c(C#N)cnn3c2)cn1]

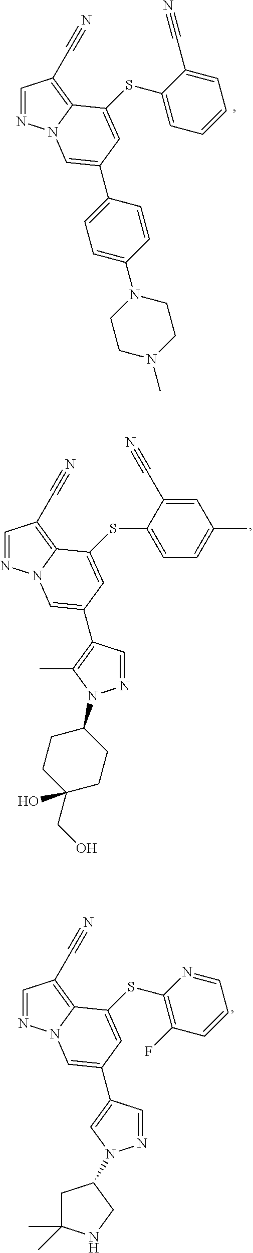 [SMILES: CN1CCN(c2ccc(-c3cc(Sc4ccccc4C#N)c4c(C#N)cnn4c3)cc2)CC1 ; Cc1ccc(Sc2cc(-c3cnn([C@H]4CC[C@](O)(CO)CC4)c3C)cn3ncc(C#N)c23)c(C#N)c1 ; CC1(C)C[C@H](n2cc(-c3cc(Sc4ncccc4F)c4c(C#N)cnn4c3)cn2)CN1]

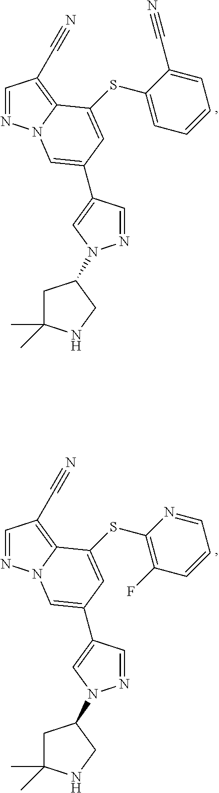 [SMILES: CC1(C)C[C@H](n2cc(-c3cc(Sc4ccccc4C#N)c4c(C#N)cnn4c3)cn2)CN1 ; CC1(C)C[C@@H](n2cc(-c3cc(Sc4ncccc4F)c4c(C#N)cnn4c3)cn2)CN1]

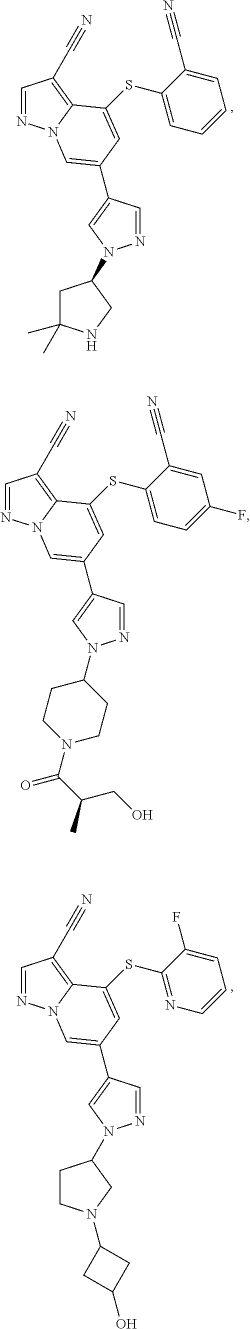 [SMILES: CC1(C)C[C@@H](n2cc(-c3cc(Sc4ccccc4C#N)c4c(C#N)cnn4c3)cn2)CN1 ; C[C@H](CO)C(=O)N1CCC(n2cc(-c3cc(Sc4ccc(F)cc4C#N)c4c(C#N)cnn4c3)cn2)CC1 ; N#Cc1cnn2cc(-c3cnn(C4CCN(C5CC(O)C5)C4)c3)cc(Sc3ncccc3F)c12]

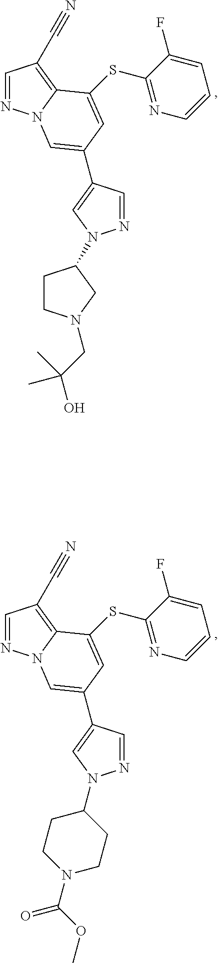 [SMILES: CC(C)(O)CN1CC[C@H](n2cc(-c3cc(Sc4ncccc4F)c4c(C#N)cnn4c3)cn2)C1 ; COC(=O)N1CCC(n2cc(-c3cc(Sc4ncccc4F)c4c(C#N)cnn4c3)cn2)CC1]

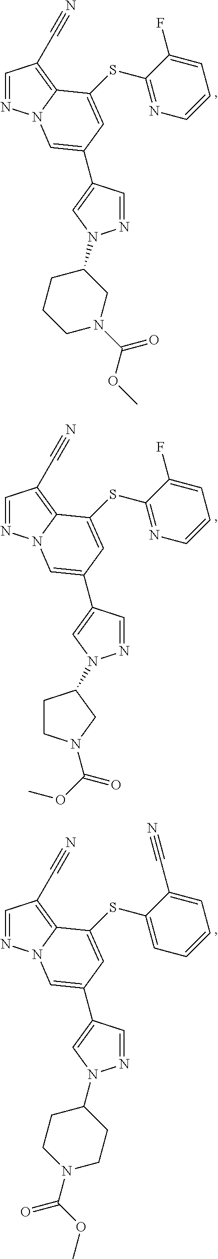

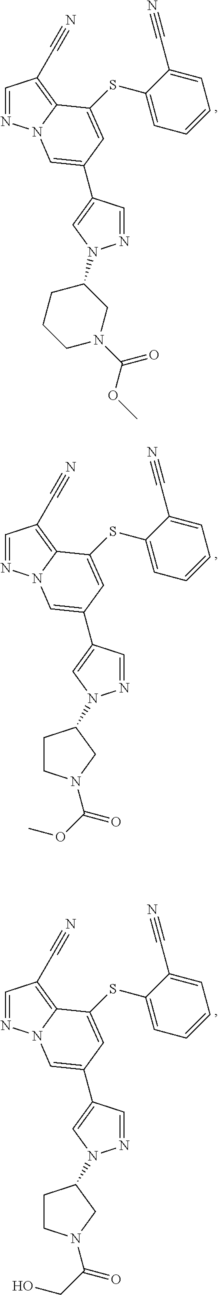

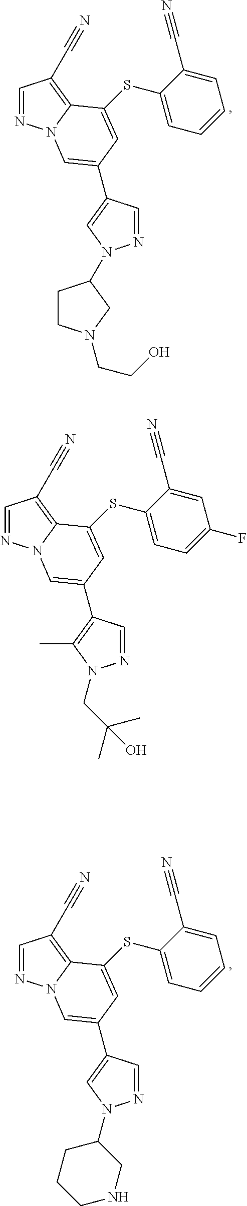 [SMILES: N#Cc1ccccc1Sc1cc(-c2cnn(C3CCN(CCO)C3)c2)cn2ncc(C#N)c12 ; Cc1c(-c2cc(Sc3ccc(F)cc3C#N)c3c(C#N)cnn3c2)cnn1CC(C)(C)O ; N#Cc1ccccc1Sc1cc(-c2cnn(C3CCCNC3)c2)cn2ncc(C#N)c12]

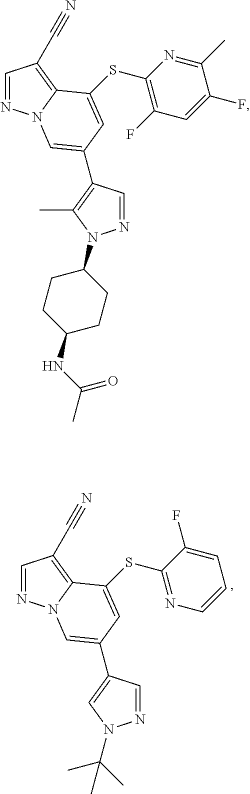 [SMILES: CC(=O)N[C@H]1CC[C@@H](n2ncc(-c3cc(Sc4nc(C)c(F)cc4F)c4c(C#N)cnn4c3)c2C)CC1 ; CC(C)(C)n1cc(-c2cc(Sc3ncccc3F)c3c(C#N)cnn3c2)cn1]

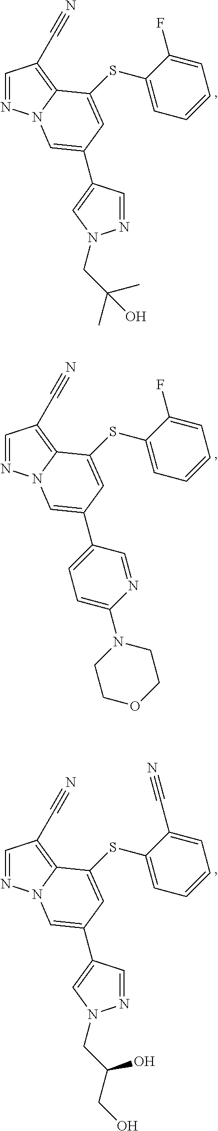 [SMILES: CC(C)(O)Cn1cc(-c2cc(Sc3ccccc3F)c3c(C#N)cnn3c2)cn1 ; N#Cc1cnn2cc(-c3ccc(N4CCOCC4)nc3)cc(Sc3ccccc3F)c12 ; N#Cc1ccccc1Sc1cc(-c2cnn(C[C@@H](O)CO)c2)cn2ncc(C#N)c12]

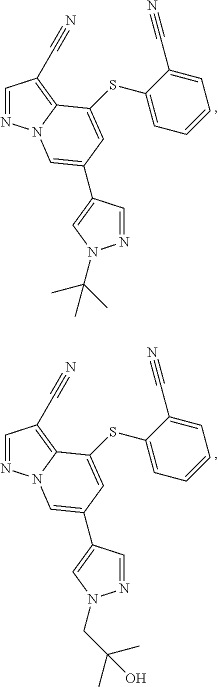 [SMILES: CC(C)(C)n1cc(-c2cc(Sc3ccccc3C#N)c3c(C#N)cnn3c2)cn1 ; CC(C)(O)Cn1cc(-c2cc(Sc3ccccc3C#N)c3c(C#N)cnn3c2)cn1]

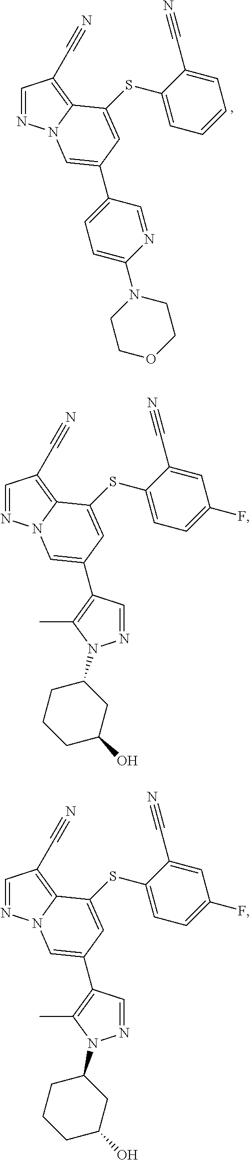

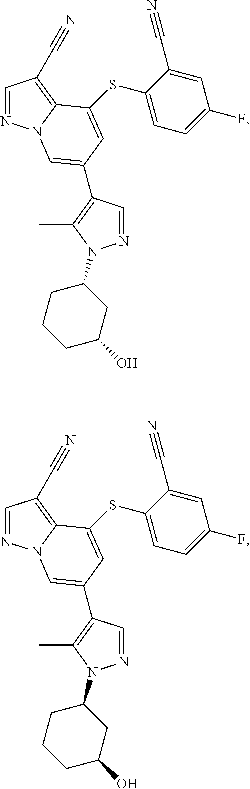 [SMILES: Cc1c(-c2cc(Sc3ccc(F)cc3C#N)c3c(C#N)cnn3c2)cnn1[C@H]1CCC[C@@H](O)C1 ; Cc1c(-c2cc(Sc3ccc(F)cc3C#N)c3c(C#N)cnn3c2)cnn1[C@@H]1CCC[C@H](O)C1]

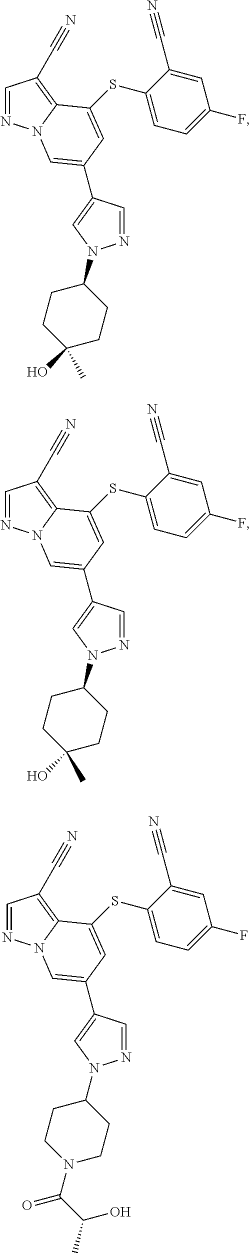 [SMILES: C[C@]1(O)CC[C@@H](n2cc(-c3cc(Sc4ccc(F)cc4C#N)c4c(C#N)cnn4c3)cn2)CC1 ; C[C@]1(O)CC[C@H](n2cc(-c3cc(Sc4ccc(F)cc4C#N)c4c(C#N)cnn4c3)cn2)CC1 ; C[C@@H](O)C(=O)N1CCC(n2cc(-c3cc(Sc4ccc(F)cc4C#N)c4c(C#N)cnn4c3)cn2)CC1]

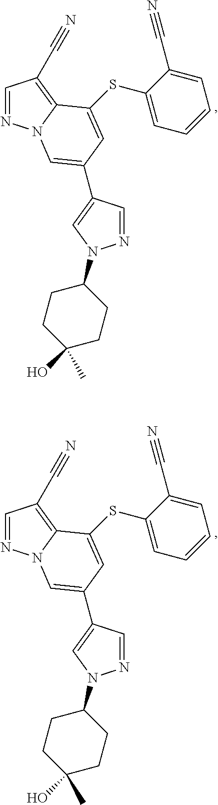 [SMILES: C[C@]1(O)CC[C@@H](n2cc(-c3cc(Sc4ccccc4C#N)c4c(C#N)cnn4c3)cn2)CC1 ; C[C@]1(O)CC[C@H](n2cc(-c3cc(Sc4ccccc4C#N)c4c(C#N)cnn4c3)cn2)CC1]

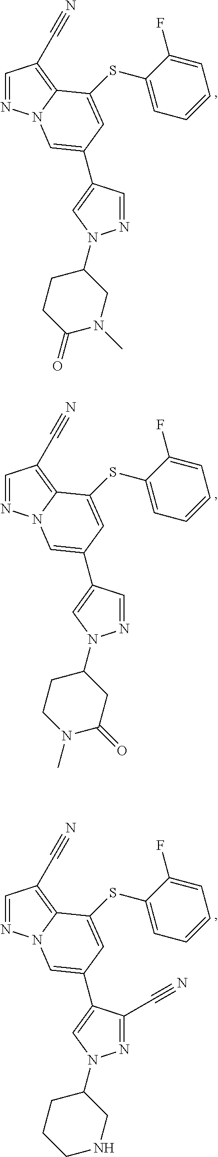

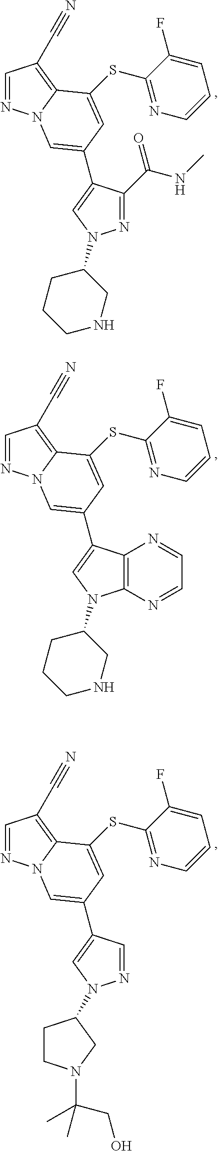

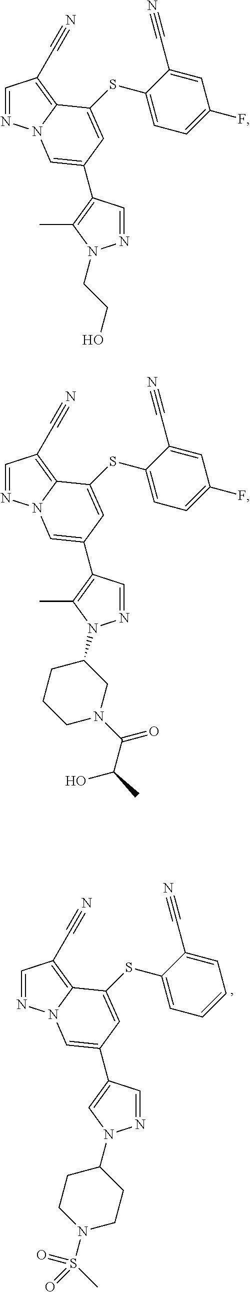 [SMILES: Cc1c(-c2cc(Sc3ccc(F)cc3C#N)c3c(C#N)cnn3c2)cnn1CCO ; Cc1c(-c2cc(Sc3ccc(F)cc3C#N)c3c(C#N)cnn3c2)cnn1[C@H]1CCCN(C(=O)[C@@H](C)O)C1 ; CS(=O)(=O)N1CCC(n2cc(-c3cc(Sc4ccccc4C#N)c4c(C#N)cnn4c3)cn2)CC1]

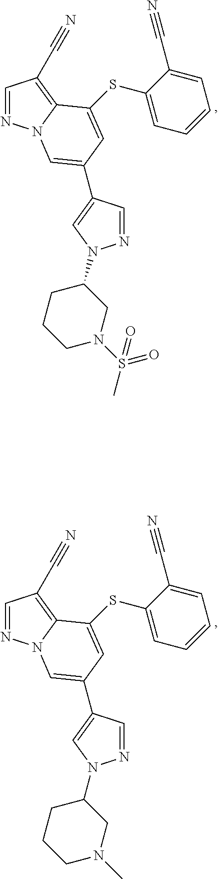 [SMILES: CS(=O)(=O)N1CCC[C@H](n2cc(-c3cc(Sc4ccccc4C#N)c4c(C#N)cnn4c3)cn2)C1 ; CN1CCCC(n2cc(-c3cc(Sc4ccccc4C#N)c4c(C#N)cnn4c3)cn2)C1]

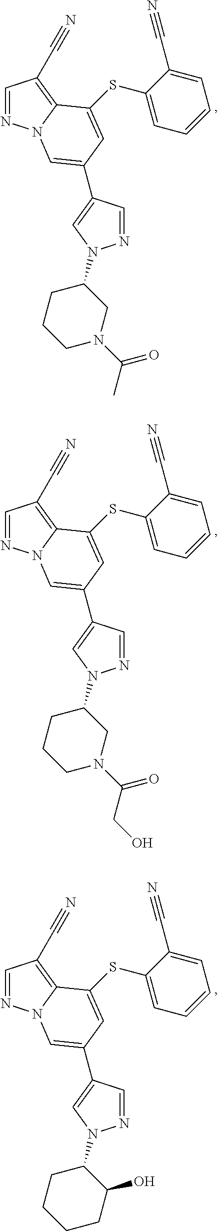 [SMILES: CC(=O)N1CCC[C@H](n2cc(-c3cc(Sc4ccccc4C#N)c4c(C#N)cnn4c3)cn2)C1 ; N#Cc1ccccc1Sc1cc(-c2cnn([C@H]3CCCN(C(=O)CO)C3)c2)cn2ncc(C#N)c12 ; N#Cc1ccccc1Sc1cc(-c2cnn([C@H]3CCCC[C@@H]3O)c2)cn2ncc(C#N)c12]

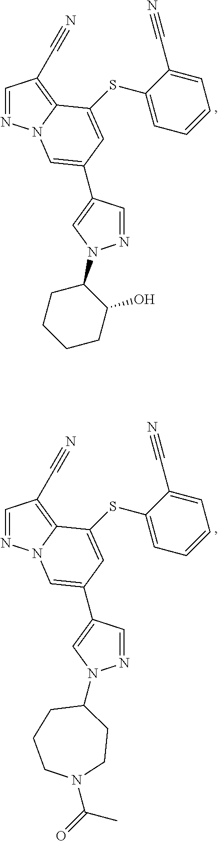 [SMILES: N#Cc1ccccc1Sc1cc(-c2cnn([C@@H]3CCCC[C@H]3O)c2)cn2ncc(C#N)c12 ; CC(=O)N1CCCC(n2cc(-c3cc(Sc4ccccc4C#N)c4c(C#N)cnn4c3)cn2)CC1]

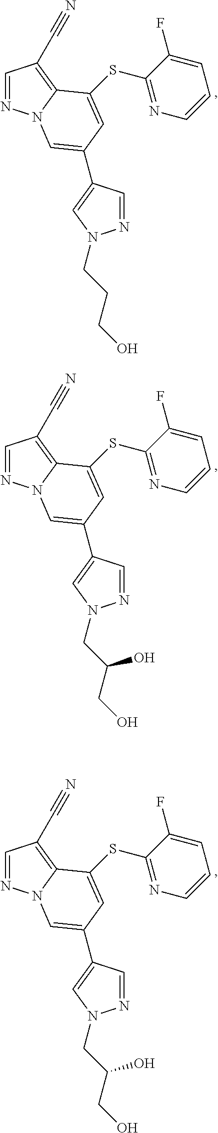 [SMILES: N#Cc1cnn2cc(-c3cnn(CCCO)c3)cc(Sc3ncccc3F)c12 ; N#Cc1cnn2cc(-c3cnn(C[C@@H](O)CO)c3)cc(Sc3ncccc3F)c12 ; N#Cc1cnn2cc(-c3cnn(C[C@H](O)CO)c3)cc(Sc3ncccc3F)c12]

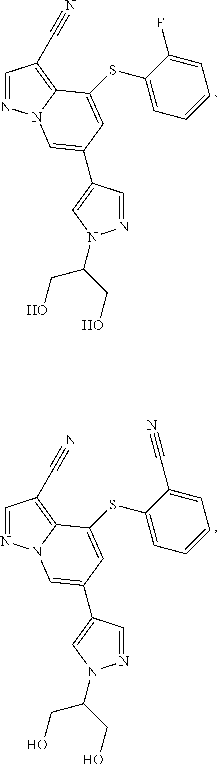 [SMILES: N#Cc1cnn2cc(-c3cnn(C(CO)CO)c3)cc(Sc3ccccc3F)c12 ; N#Cc1ccccc1Sc1cc(-c2cnn(C(CO)CO)c2)cn2ncc(C#N)c12]

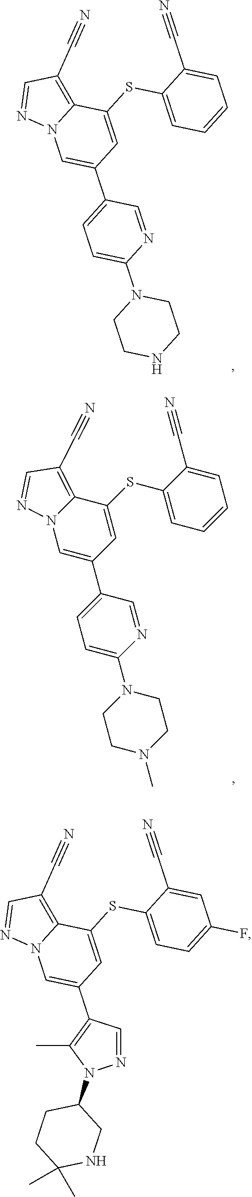

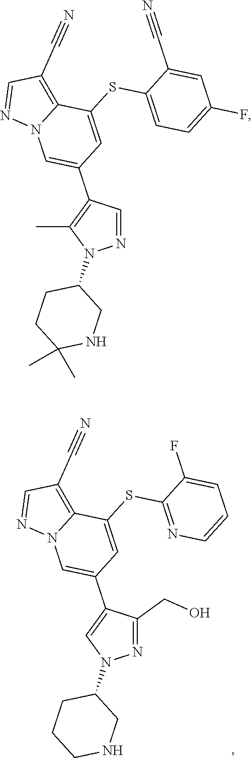 [SMILES: Cc1c(-c2cc(Sc3ccc(F)cc3C#N)c3c(C#N)cnn3c2)cnn1[C@H]1CCC(C)(C)NC1 ; N#Cc1cnn2cc(-c3cn([C@H]4CCCNC4)nc3CO)cc(Sc3ncccc3F)c12]

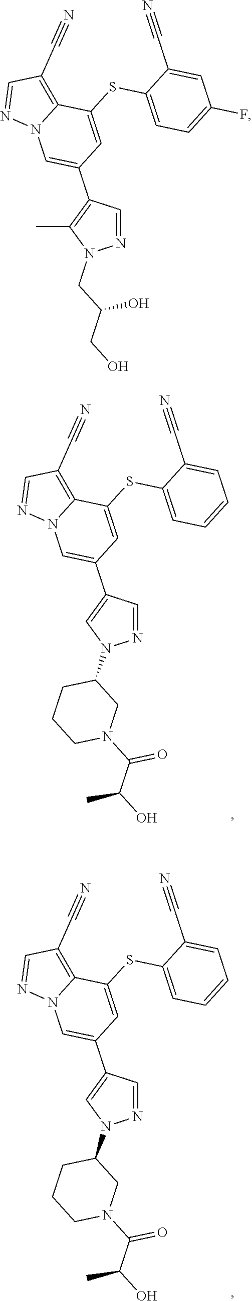 [SMILES: Cc1c(-c2cc(Sc3ccc(F)cc3C#N)c3c(C#N)cnn3c2)cnn1C[C@H](O)CO ; C[C@H](O)C(=O)N1CCC[C@H](n2cc(-c3cc(Sc4ccccc4C#N)c4c(C#N)cnn4c3)cn2)C1 ; C[C@H](O)C(=O)N1CCC[C@@H](n2cc(-c3cc(Sc4ccccc4C#N)c4c(C#N)cnn4c3)cn2)C1]

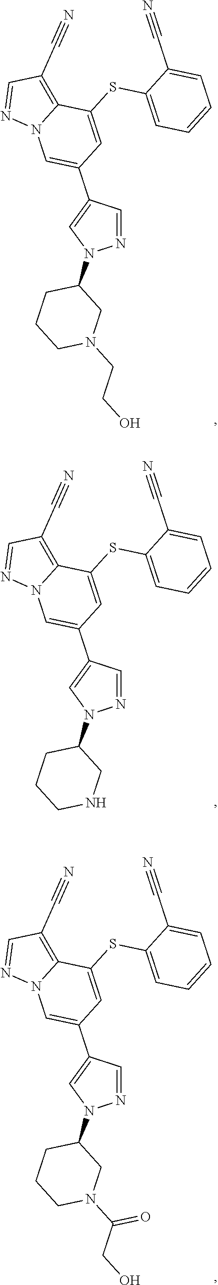 [SMILES: N#Cc1ccccc1Sc1cc(-c2cnn([C@@H]3CCCN(CCO)C3)c2)cn2ncc(C#N)c12 ; N#Cc1ccccc1Sc1cc(-c2cnn([C@@H]3CCCNC3)c2)cn2ncc(C#N)c12 ; N#Cc1ccccc1Sc1cc(-c2cnn([C@@H]3CCCN(C(=O)CO)C3)c2)cn2ncc(C#N)c12]

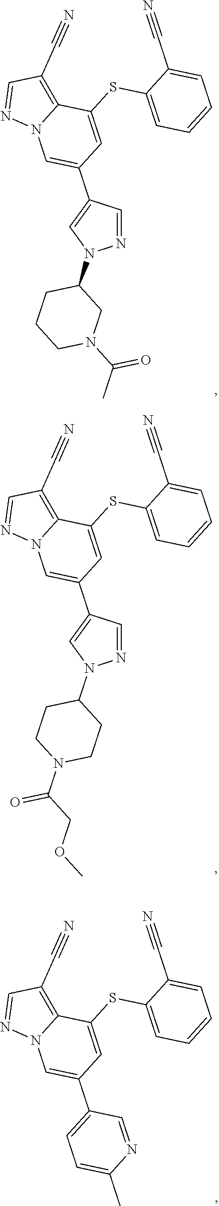

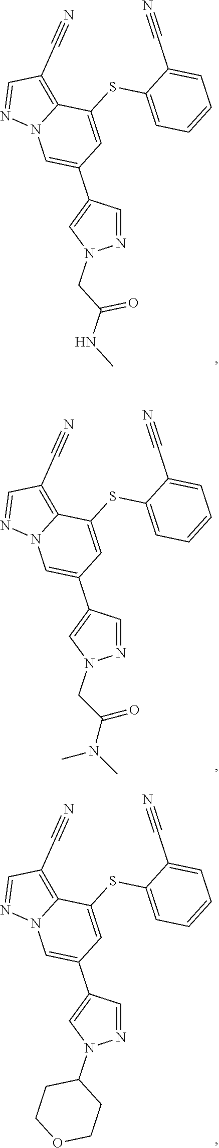

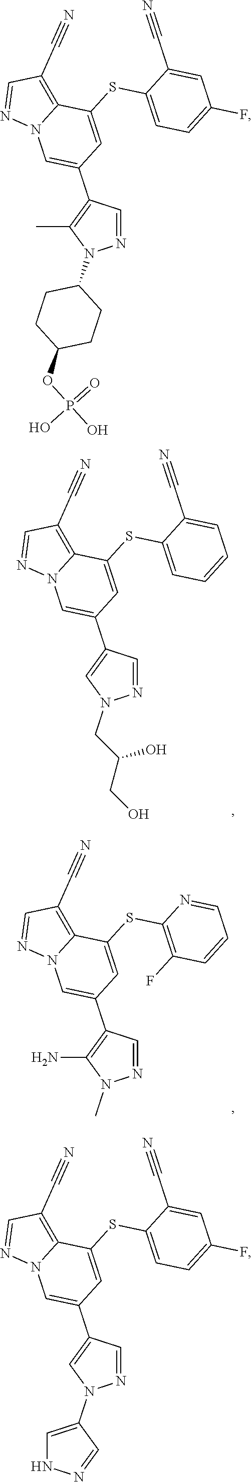

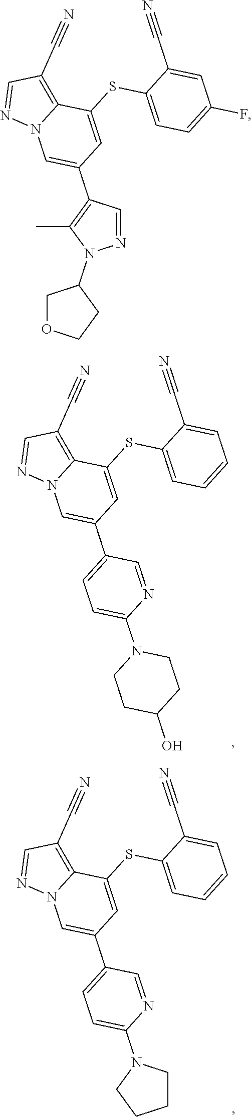

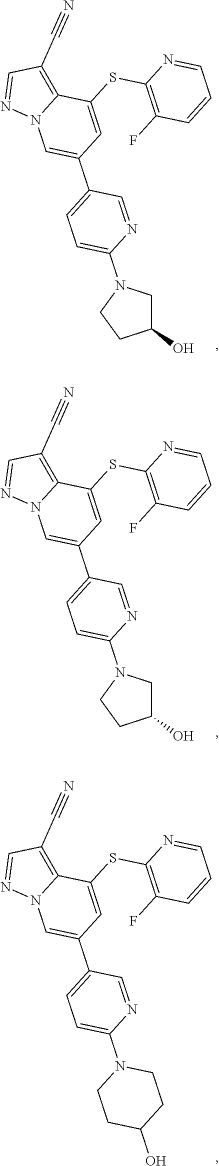 [SMILES: N#Cc1cnn2cc(-c3ccc(N4CC[C@H](O)C4)nc3)cc(Sc3ncccc3F)c12 ; N#Cc1cnn2cc(-c3ccc(N4CC[C@@H](O)C4)nc3)cc(Sc3ncccc3F)c12 ; N#Cc1cnn2cc(-c3ccc(N4CCC(O)CC4)nc3)cc(Sc3ncccc3F)c12]

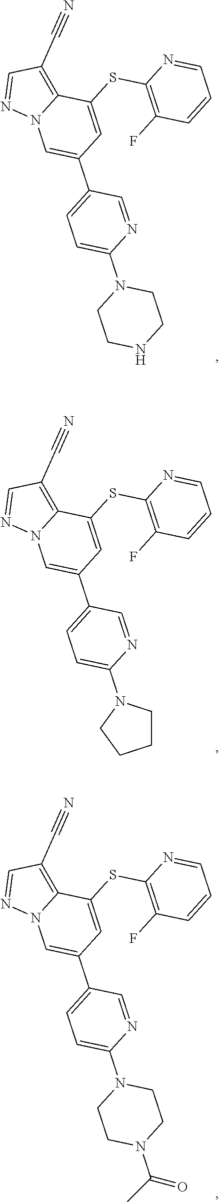 [SMILES: N#Cc1cnn2cc(-c3ccc(N4CCNCC4)nc3)cc(Sc3ncccc3F)c12 ; N#Cc1cnn2cc(-c3ccc(N4CCCC4)nc3)cc(Sc3ncccc3F)c12 ; CC(=O)N1CCN(c2ccc(-c3cc(Sc4ncccc4F)c4c(C#N)cnn4c3)cn2)CC1]

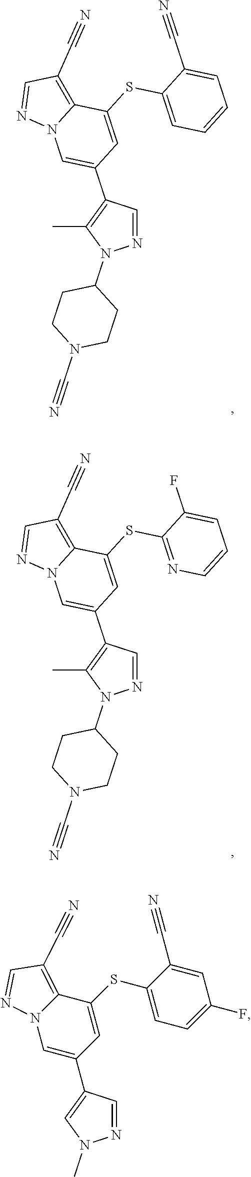 [SMILES: Cc1c(-c2cc(Sc3ccccc3C#N)c3c(C#N)cnn3c2)cnn1C1CCN(C#N)CC1 ; Cc1c(-c2cc(Sc3ncccc3F)c3c(C#N)cnn3c2)cnn1C1CCN(C#N)CC1 ; Cn1cc(-c2cc(Sc3ccc(F)cc3C#N)c3c(C#N)cnn3c2)cn1]

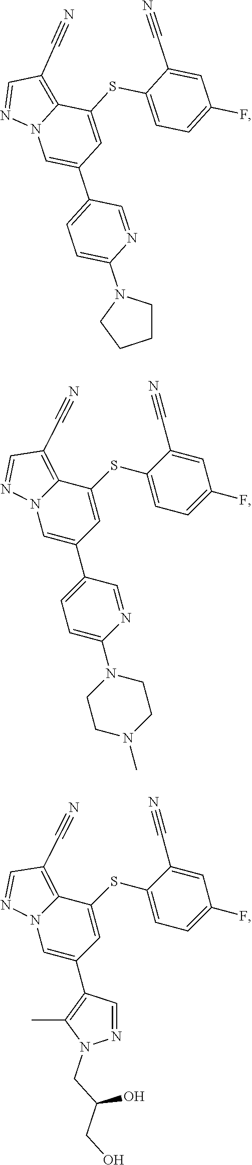

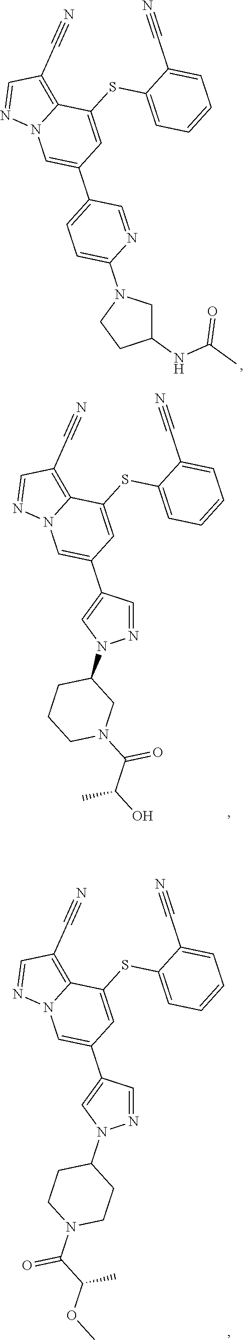

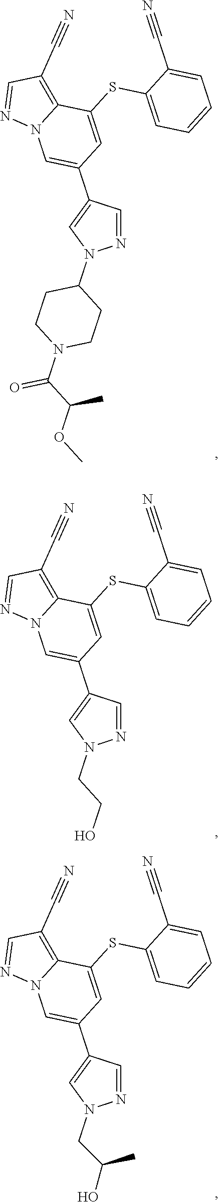

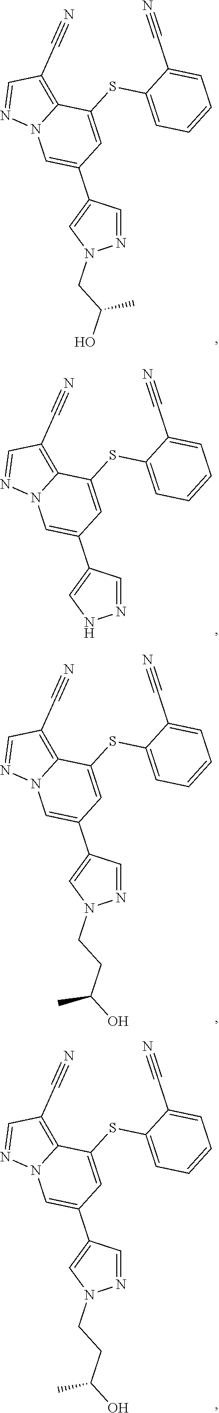

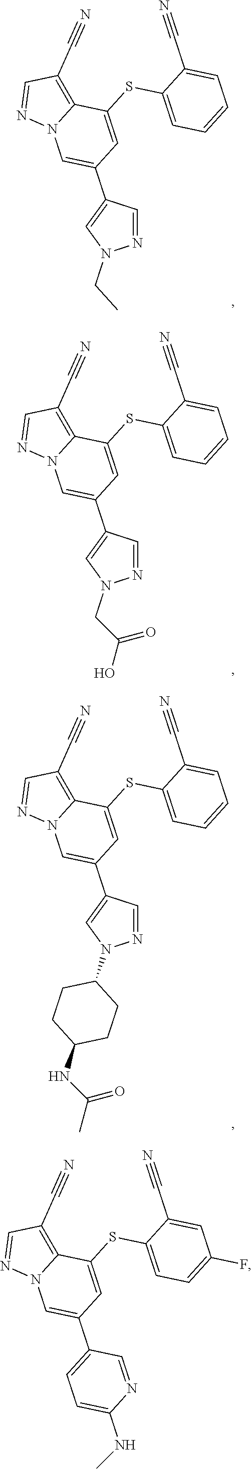

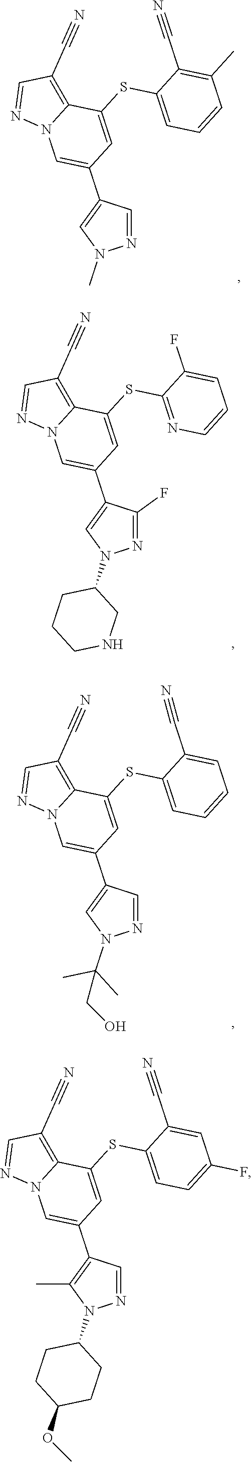

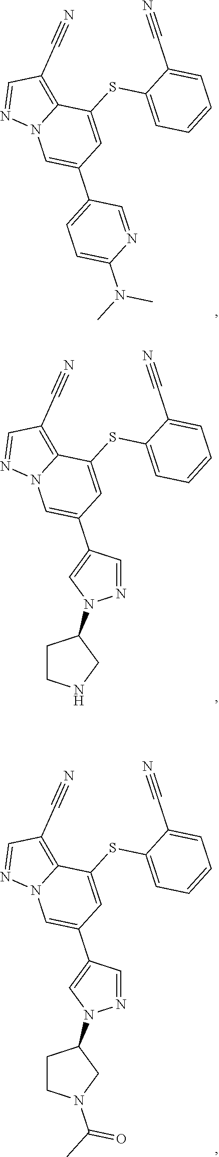 [SMILES: CN(C)c1ccc(-c2cc(Sc3ccccc3C#N)c3c(C#N)cnn3c2)cn1 ; N#Cc1ccccc1Sc1cc(-c2cnn([C@@H]3CCNC3)c2)cn2ncc(C#N)c12 ; CC(=O)N1CC[C@@H](n2cc(-c3cc(Sc4ccccc4C#N)c4c(C#N)cnn4c3)cn2)C1]

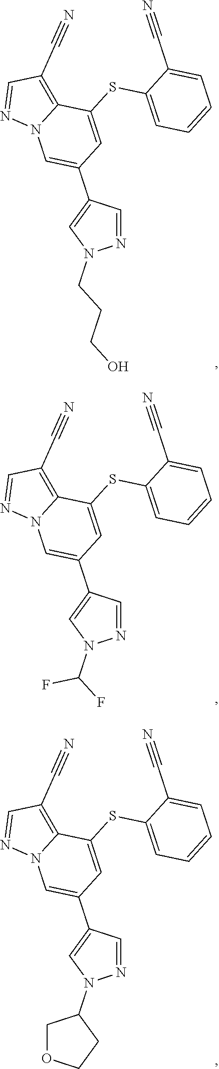

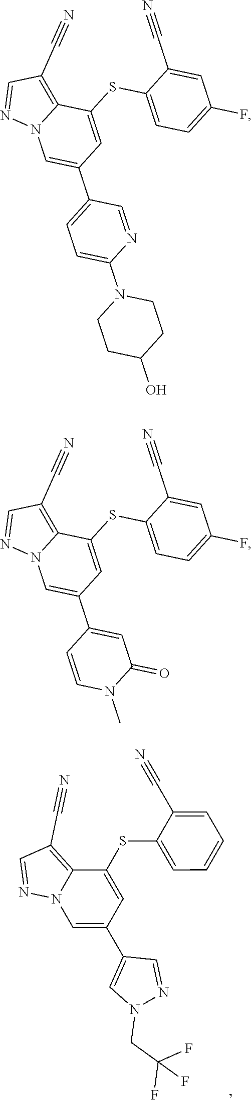

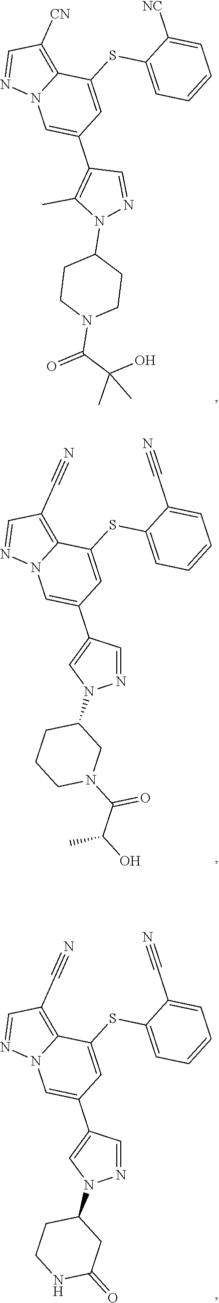 [SMILES: Cc1c(-c2cc(Sc3ccccc3C#N)c3c(C#N)cnn3c2)cnn1C1CCN(C(=O)C(C)(C)O)CC1 ; C[C@@H](O)C(=O)N1CCC[C@H](n2cc(-c3cc(Sc4ccccc4C#N)c4c(C#N)cnn4c3)cn2)C1 ; N#Cc1ccccc1Sc1cc(-c2cnn([C@@H]3CCNC(=O)C3)c2)cn2ncc(C#N)c12]

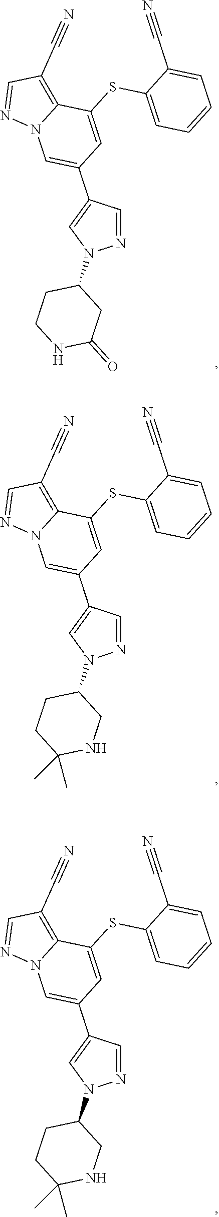 [SMILES: N#Cc1ccccc1Sc1cc(-c2cnn([C@H]3CCNC(=O)C3)c2)cn2ncc(C#N)c12 ; CC1(C)CC[C@H](n2cc(-c3cc(Sc4ccccc4C#N)c4c(C#N)cnn4c3)cn2)CN1 ; CC1(C)CC[C@@H](n2cc(-c3cc(Sc4ccccc4C#N)c4c(C#N)cnn4c3)cn2)CN1]

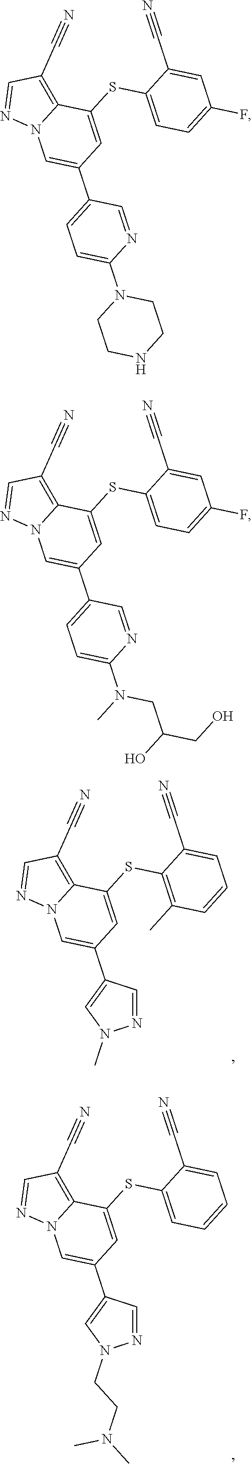

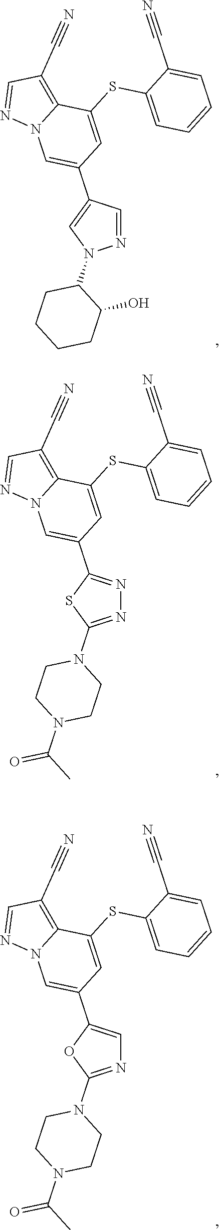

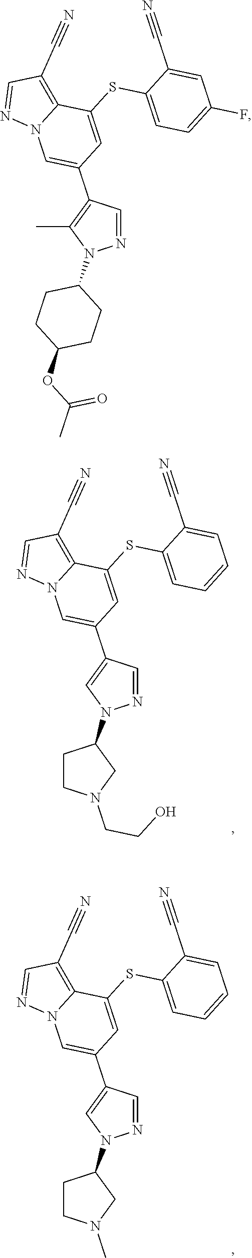

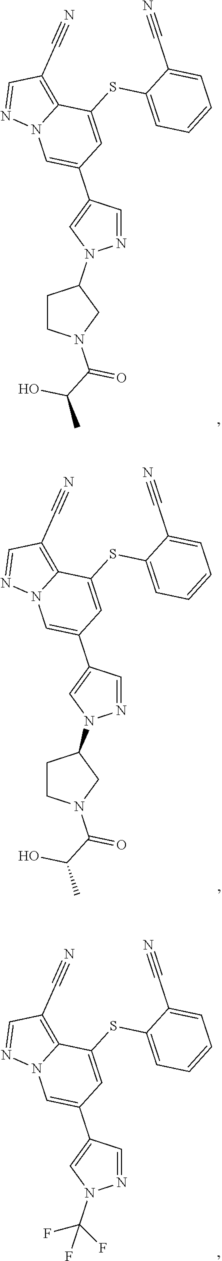 [SMILES: C[C@@H](O)C(=O)N1CCC(n2cc(-c3cc(Sc4ccccc4C#N)c4c(C#N)cnn4c3)cn2)C1 ; C[C@H](O)C(=O)N1CC[C@@H](n2cc(-c3cc(Sc4ccccc4C#N)c4c(C#N)cnn4c3)cn2)C1 ; N#Cc1ccccc1Sc1cc(-c2cnn(C(F)(F)F)c2)cn2ncc(C#N)c12]

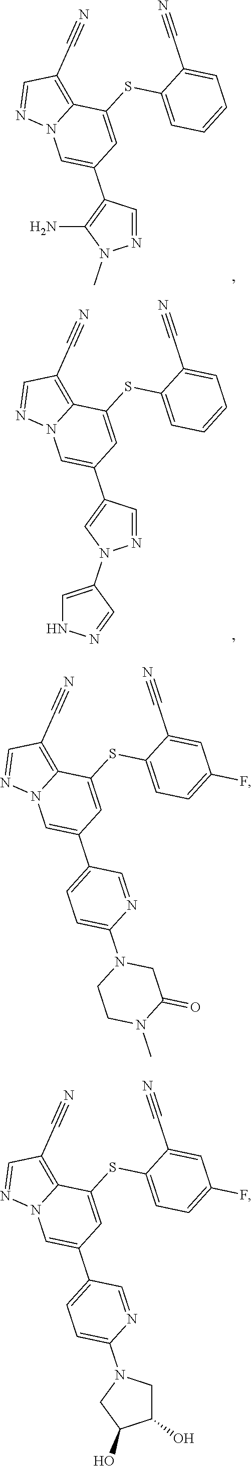

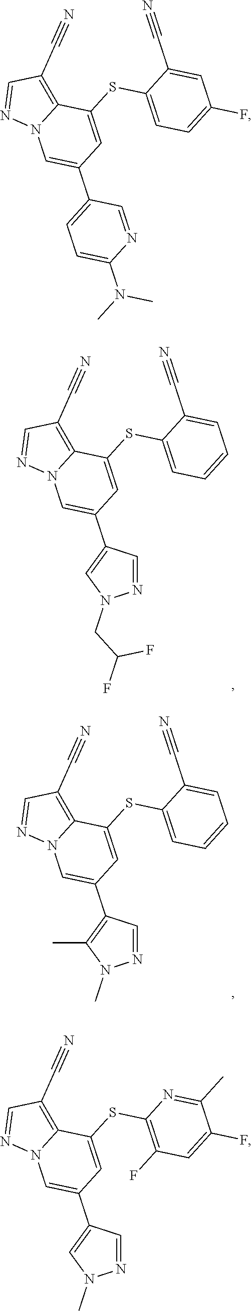

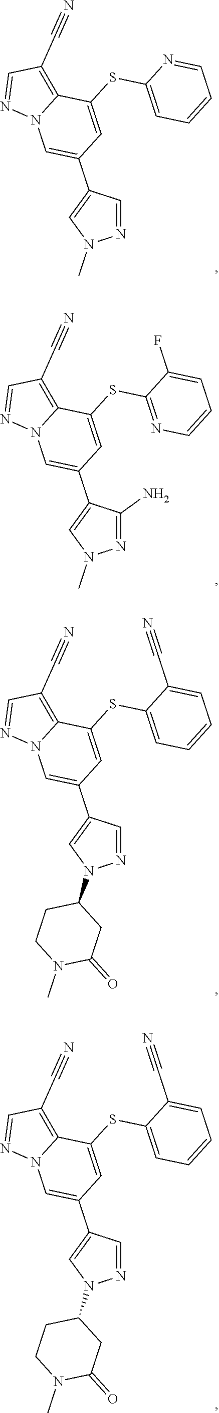

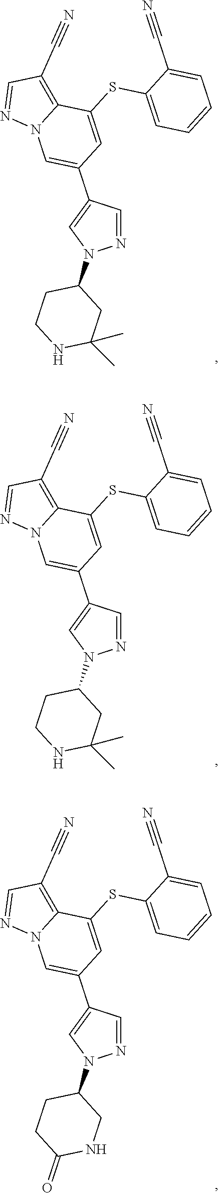 [SMILES: CC1(C)C[C@H](n2cc(-c3cc(Sc4ccccc4C#N)c4c(C#N)cnn4c3)cn2)CCN1 ; CC1(C)C[C@@H](n2cc(-c3cc(Sc4ccccc4C#N)c4c(C#N)cnn4c3)cn2)CCN1 ; N#Cc1ccccc1Sc1cc(-c2cnn([C@@H]3CCC(=O)NC3)c2)cn2ncc(C#N)c12]

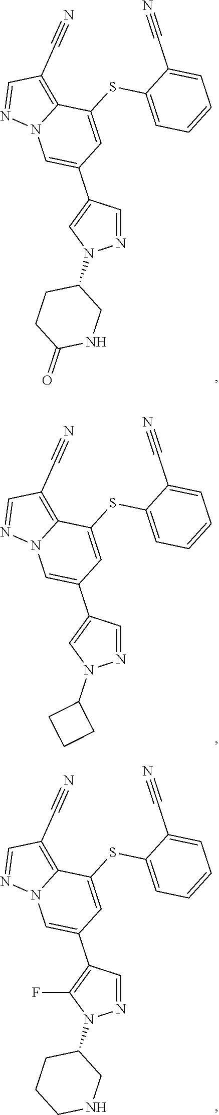

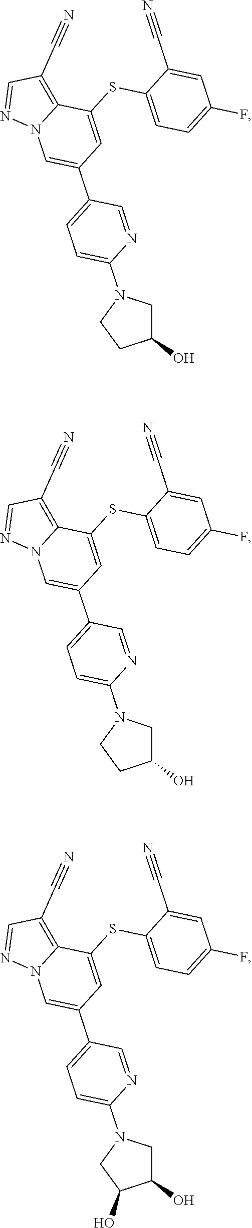 [SMILES: N#Cc1cc(F)ccc1Sc1cc(-c2ccc(N3CC[C@H](O)C3)nc2)cn2ncc(C#N)c12 ; N#Cc1cc(F)ccc1Sc1cc(-c2ccc(N3CC[C@@H](O)C3)nc2)cn2ncc(C#N)c12 ; N#Cc1cc(F)ccc1Sc1cc(-c2ccc(N3C[C@@H](O)[C@@H](O)C3)nc2)cn2ncc(C#N)c12]

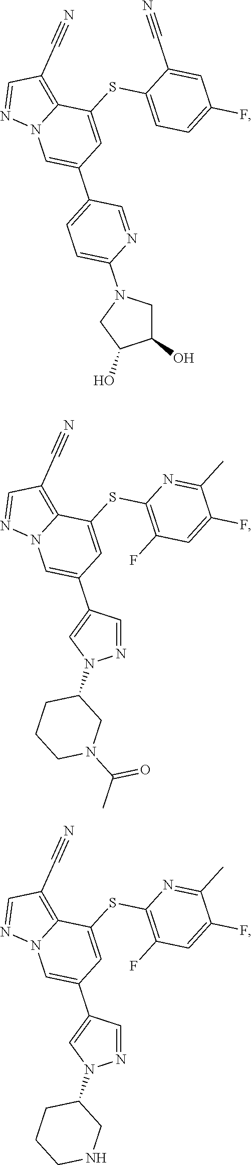

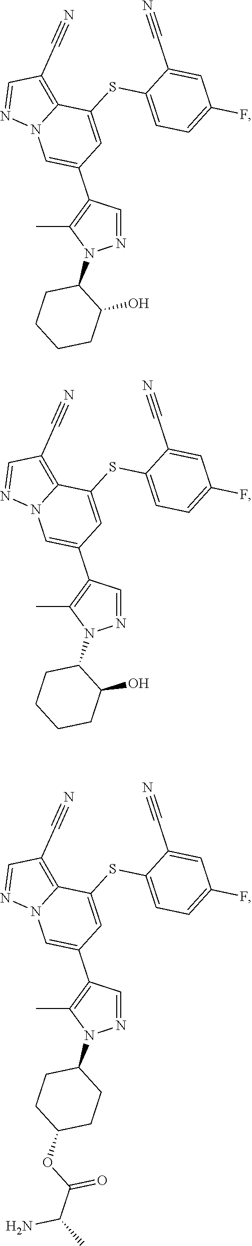 [SMILES: Cc1c(-c2cc(Sc3ccc(F)cc3C#N)c3c(C#N)cnn3c2)cnn1[C@@H]1CCCC[C@H]1O ; Cc1c(-c2cc(Sc3ccc(F)cc3C#N)c3c(C#N)cnn3c2)cnn1[C@H]1CCCC[C@@H]1O ; Cc1c(-c2cc(Sc3ccc(F)cc3C#N)c3c(C#N)cnn3c2)cnn1[C@H]1CC[C@H](OC(=O)[C@H](C)N)CC1]

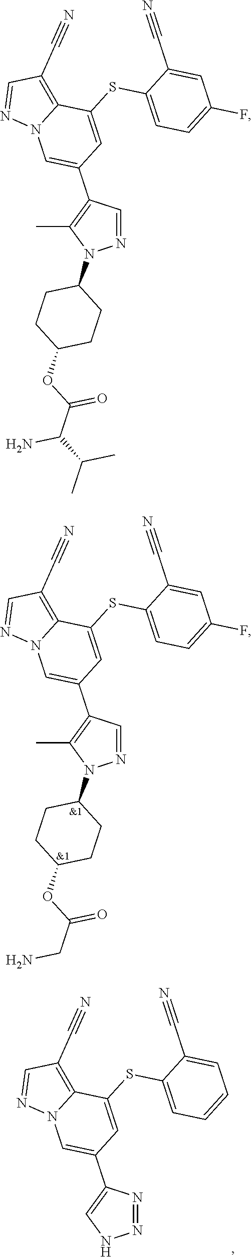

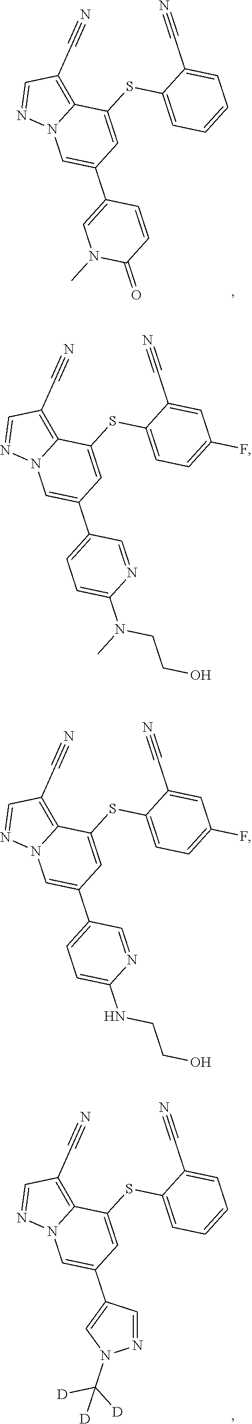

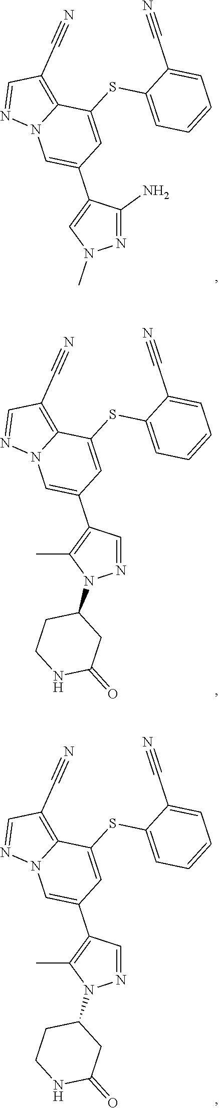 [SMILES: Cn1cc(-c2cc(Sc3ccccc3C#N)c3c(C#N)cnn3c2)c(N)n1 ; Cc1c(-c2cc(Sc3ccccc3C#N)c3c(C#N)cnn3c2)cnn1[C@@H]1CCNC(=O)C1 ; Cc1c(-c2cc(Sc3ccccc3C#N)c3c(C#N)cnn3c2)cnn1[C@H]1CCNC(=O)C1]

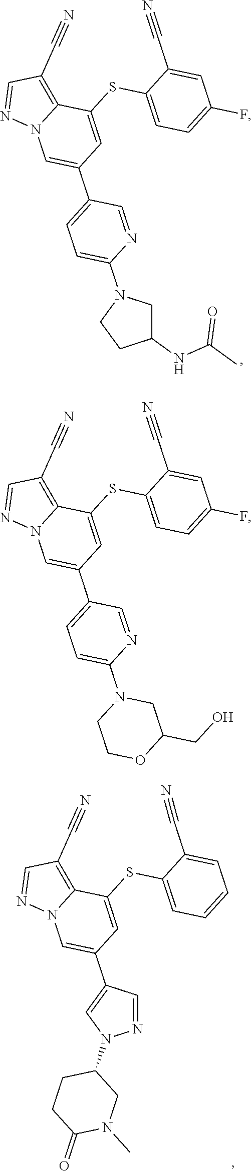

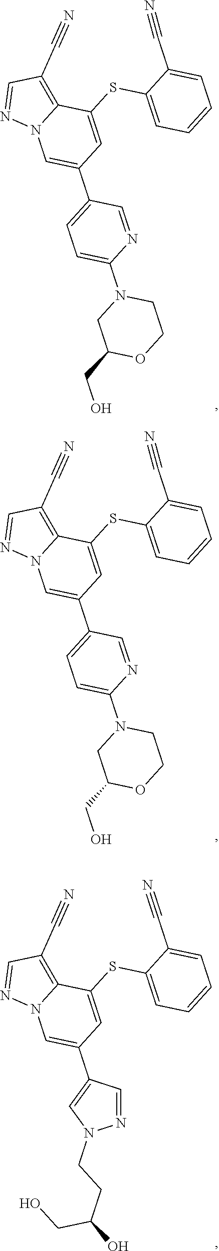 [SMILES: N#Cc1ccccc1Sc1cc(-c2ccc(N3CCO[C@@H](CO)C3)nc2)cn2ncc(C#N)c12 ; N#Cc1ccccc1Sc1cc(-c2ccc(N3CCO[C@H](CO)C3)nc2)cn2ncc(C#N)c12 ; N#Cc1ccccc1Sc1cc(-c2cnn(CC[C@@H](O)CO)c2)cn2ncc(C#N)c12]

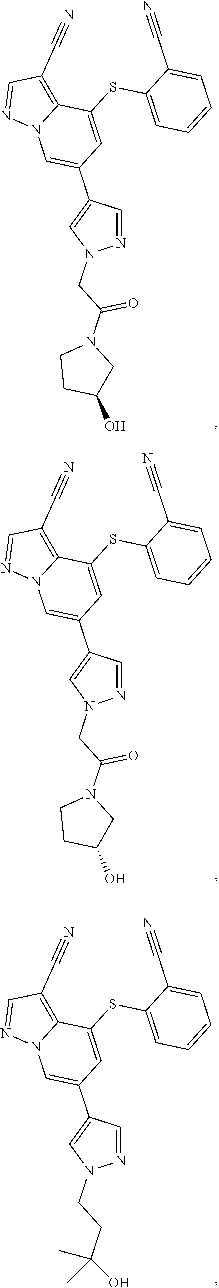 [SMILES: N#Cc1ccccc1Sc1cc(-c2cnn(CC(=O)N3CC[C@H](O)C3)c2)cn2ncc(C#N)c12 ; N#Cc1ccccc1Sc1cc(-c2cnn(CC(=O)N3CC[C@@H](O)C3)c2)cn2ncc(C#N)c12 ; CC(C)(O)CCn1cc(-c2cc(Sc3ccccc3C#N)c3c(C#N)cnn3c2)cn1]

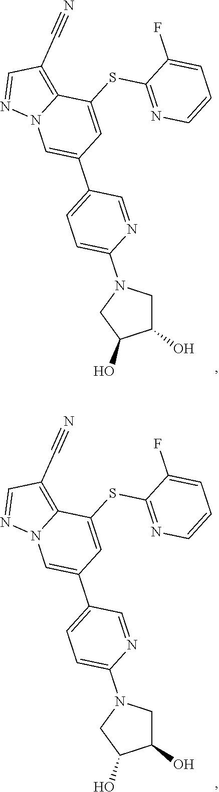 [SMILES: N#Cc1cnn2cc(-c3ccc(N4C[C@H](O)[C@@H](O)C4)nc3)cc(Sc3ncccc3F)c12 ; N#Cc1cnn2cc(-c3ccc(N4C[C@@H](O)[C@H](O)C4)nc3)cc(Sc3ncccc3F)c12]

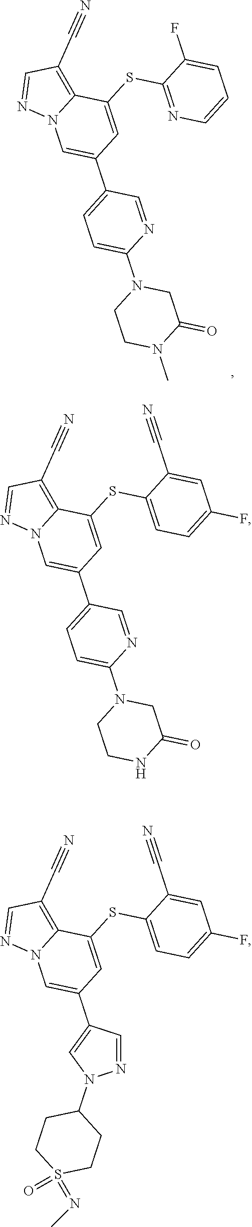

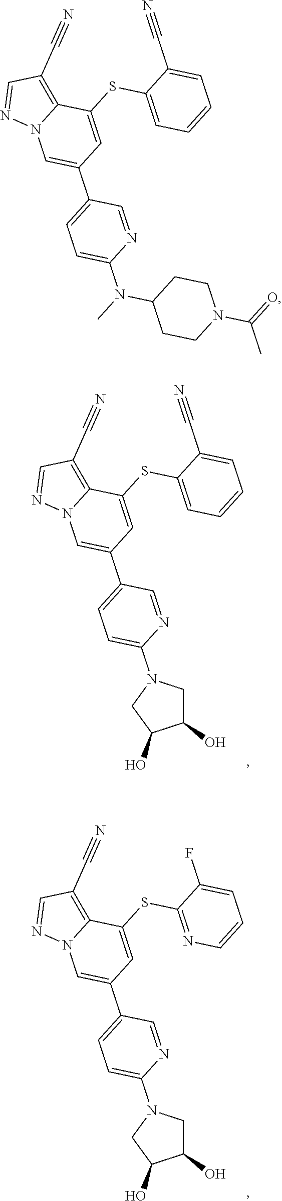 [SMILES: CC(=O)N1CCC(N(C)c2ccc(-c3cc(Sc4ccccc4C#N)c4c(C#N)cnn4c3)cn2)CC1 ; N#Cc1ccccc1Sc1cc(-c2ccc(N3C[C@@H](O)[C@@H](O)C3)nc2)cn2ncc(C#N)c12 ; N#Cc1cnn2cc(-c3ccc(N4C[C@@H](O)[C@@H](O)C4)nc3)cc(Sc3ncccc3F)c12]

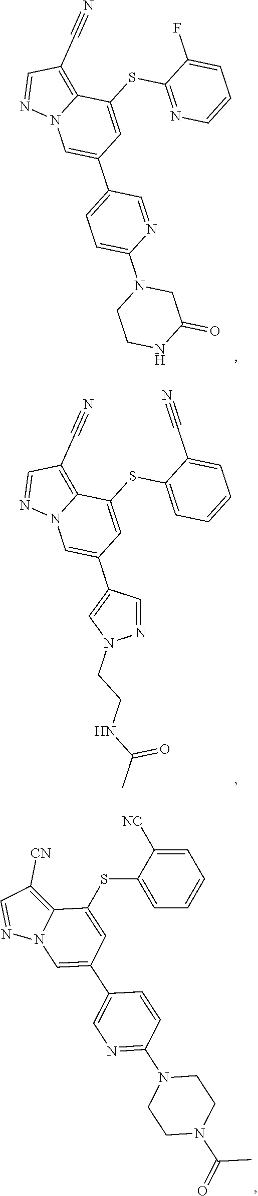

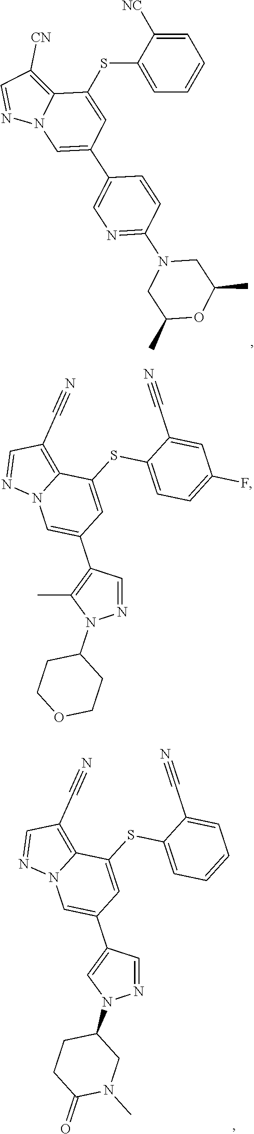

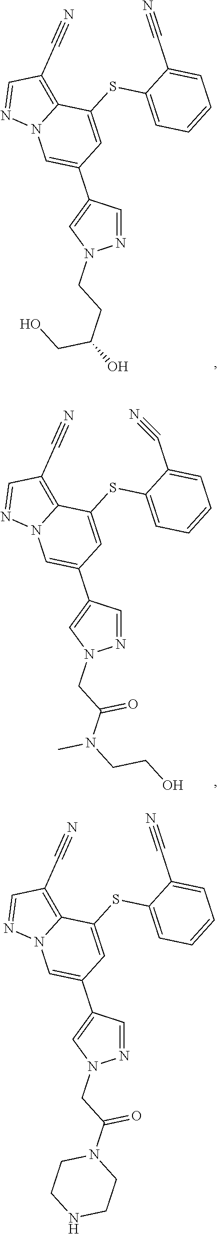

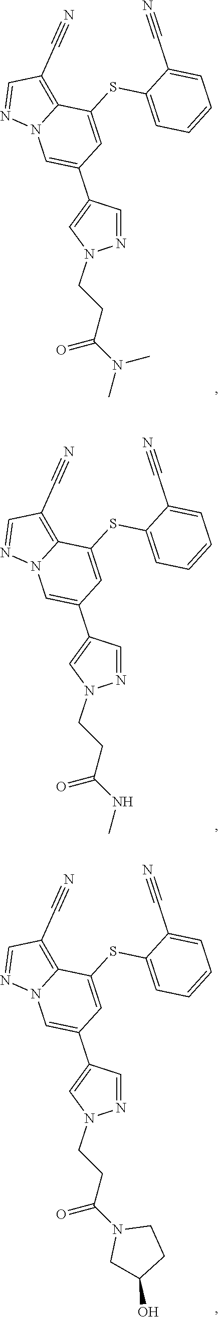 [SMILES: CN(C)C(=O)CCn1cc(-c2cc(Sc3ccccc3C#N)c3c(C#N)cnn3c2)cn1 ; CNC(=O)CCn1cc(-c2cc(Sc3ccccc3C#N)c3c(C#N)cnn3c2)cn1 ; N#Cc1ccccc1Sc1cc(-c2cnn(CCC(=O)N3CC[C@@H](O)C3)c2)cn2ncc(C#N)c12]

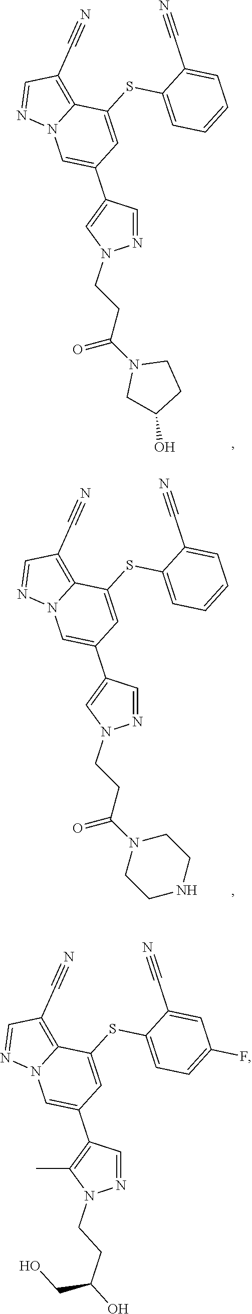

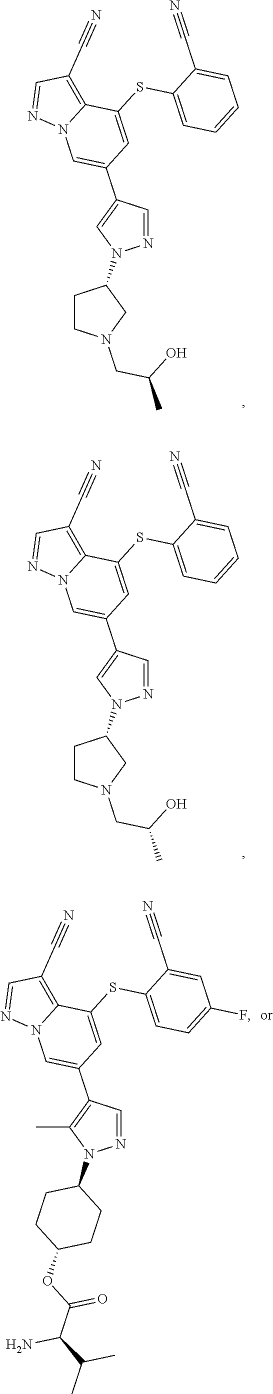 [SMILES: C[C@H](O)CN1CC[C@H](n2cc(-c3cc(Sc4ccccc4C#N)c4c(C#N)cnn4c3)cn2)C1 ; C[C@@H](O)CN1CC[C@H](n2cc(-c3cc(Sc4ccccc4C#N)c4c(C#N)cnn4c3)cn2)C1 ; Cc1c(-c2cc(Sc3ccc(F)cc3C#N)c3c(C#N)cnn3c2)cnn1[C@H]1CC[C@H](OC(=O)[C@H](N)C(C)C)CC1 ; "or"]

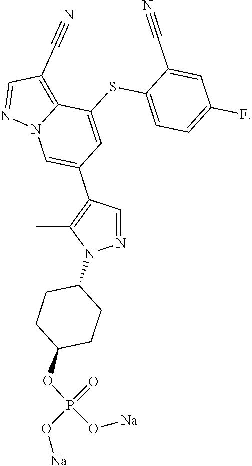

66. A pharmaceutical composition comprising the compound of claim 1 and a therapeutic agent.

67. The compound of any one of claims 1-65 or the pharmaceutical composition of claim 66 for use in treating a disease associated with mutations in fibroblast growth factor receptor 2 (FGFR2) or fibroblast growth factor receptor 3 (FGFR3).

68. A method of treating a disease associated with mutations in FGFR2 or FGFR3, comprising: administering the compound of any one of claims 1-65 or the pharmaceutical composition of claim 66 to a subject in need thereof.

69. The method of claim 68, wherein the subject is an animal.

70. The method of any one of claims 68-69, wherein the subject is a human.

71. The method of any one of claims 68-70, wherein the disease associated with mutations in FGFR2 is a cancer or craniosynostoic syndrome.

72. The method of any one of claims 68-71, wherein the cancer is intrahepatic cholangiocarcinoma, hepatocellular carcinoma, breast cancer, prostate cancer, lung squamous cell carcinoma, thyroid cancer, gastric cancer, ovarian cancer, endometrial cancer, non-small cell lung cancer, urothelial cancers, colorectal cancer, colon cancer, metastatic cholangiocarcinoma, cholangiocarcinoma, osteosarcoma, gastroesophageal junction adenocarcinoma, biliary tract cancer, anaplastic thyroid carcinoma, ganglioglioma, pancreatic intraductal tubulopapillary neoplasm, gallbladder carcinoma, renal cell carcinoma, myxoid lipocarcinoma, triple negative breast cancer, or rectal cancer.

73. The method of any one of claims 68-71, wherein the craniosynostoic syndrome is craniosynostosis, bent bone dysplasia, crouzon syndrome, apert syndrome, pfeiffer syndrome, antley-bixler, beare-stevenson syndrome, jackson-weiss syndrome, or seathre-chotzen-like syndromes.

74. The method of any one of claims 68-70, wherein the disease associated with mutations in FGFR3 is systemic sclerosis, fibrosis, pulmonary fibrosis, achondroplasia, thanatophoric dysplasia, severe achondroplasia with developmental delay and acanthosis nigricans (SADDAN syndrome), muenke syndrome, FGFR3 associated cancer, hypochondroplasia, FGFR3 related craniosynostosis, LADD syndrome, or Alzheimer disease.
